(19) United States
(12) Patent Application Publication
(10) Pub. No.: US 2024/0199647 A1
(43) Pub. Date: Jun. 20, 2024

(54) ANTI-VIRAL COMPOUNDS

(71) Applicants: Aligos Therapeutics, Inc., South San Francisco, CA (US); Katholieke Universiteit Leuven, Leuven (BE)

(72) Inventors: Dorothée Alice Marie-Eve Bardiot, Leuven (BE); Sandro Boland, Leuven (BE); Arnaud Didier Marie Marchand, Leuven (BE); Koen Vandyck, Paal (BE); David McGowan, Brussels (BE); Leonid Beigelman, San Mateo, CA (US); Antitsa Dimitrova Stoycheva, Half Moon Bay, CA (US)

(21) Appl. No.: 18/499,941

(22) Filed: Nov. 1, 2023

Related U.S. Application Data
(60) Provisional application No. 63/512,854, filed on Jul. 10, 2023.
(60) Provisional application No. 63/382,078, filed on Nov. 2, 2022.

(51) Int. Cl. C07D 513/10; A61K 45/06; A61P 31/14; C07D 471/10; C07D 487/10; C07D 487/20; C07D 498/10; C07D 498/20

(52) U.S. Cl. CPC C07D 513/10; A61K 45/06; A61P 31/14; C07D 471/10; C07D 487/10; C07D 487/20; C07D 498/10; C07D 498/20

(57) ABSTRACT

Provided herein are compounds of Formula (I), or pharmaceutically acceptable salts thereof, pharmaceutical compositions that include a compound described herein (including pharmaceutically acceptable salts of a compound described herein) and methods of synthesizing the same. Also provided herein are methods of treating diseases and/or conditions with a compound of Formula (I), or a pharmaceutically acceptable salt thereof.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference in their entireties under 37 CFR 1.57, and Rules 4.18 and 20.6, including U.S. Provisional Application Nos. 63/382,078, filed Nov. 2, 2022 and 63/512,854, filed Jul. 10, 2023.

### BACKGROUND

### Field

The present application relates to the fields of chemistry, biochemistry and medicine. Disclosed herein are compounds of Formula (I), or pharmaceutically acceptable salt thereof, pharmaceutical compositions that include a compound described herein (including pharmaceutically acceptable salts of a compound described herein) and methods of synthesizing the same. Also disclosed herein are methods of treating diseases and/or conditions with a compound of Formula (I), or a pharmaceutically acceptable salt thereof.

### Description

A positive-sense single-stranded RNA virus ((+)ssRNA virus) is a virus that uses positive sense, single stranded, RNA as its genetic material. Positive-sense single-stranded RNA viruses can be enveloped or non-enveloped. Coronaviridae, Picornaviridae and Norviruses are each a (+)ssRNA virus. Each of the aforementioned viruses are known to infect mammals, including humans.

### SUMMARY

Some embodiments disclosed herein relate to a compound of Formula (I), or a pharmaceutically acceptable salt thereof.

Some embodiments disclosed herein relate to a pharmaceutical composition that can contain an effective amount of a compound of Formula (I), or a pharmaceutically acceptable salt thereof.

Some embodiments described herein relate to a method of treating a coronavirus infection that can include administering to a subject identified as suffering from the coronavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a coronavirus infection.

Some embodiments disclosed herein relate to a method of inhibiting replication of a coronavirus that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a coronavirus.

Some embodiments described herein relate to a method of treating a picornavirus infection that can include administering to a subject identified as suffering from the picornavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a picornavirus infection.

Some embodiments disclosed herein relate to a method of inhibiting replication of a picornavirus that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a picornavirus.

Some embodiments described herein relate to a method of treating a norovirus infection that can include administering to a subject identified as suffering from the norovirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a norovirus infection.

Some embodiments disclosed herein relate to a method of inhibiting replication of a norovirus that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a norovirus.

These are other embodiments are described in greater detail below.

### DETAILED DESCRIPTION

Coronaviridae viruses are a family of enveloped, positive-stranded, single-stranded, spherical RNA viruses. Coronaviruses are named for the crown-like spikes on their surface. The Coronaviridae family includes two sub-families, Coronavirus and Torovirus. The Coronavirus genus has a helical nucleocapsid, and Torovirus genus has a tubular nucleocapsid. The Coronaviridae family of viruses includes Middle East respiratory syndrome coronavirus (MERS-CoV), SARS and SARS-CoV-2.

Coronavirus disease 2019 (COVID-19) (also referred to as novel coronavirus pneumonia or 2019-nCoV acute respiratory disease) is an infectious disease caused by the virus severe acute respiratory syndrome coronavirus 2 (SARS-CoV-2) (also referred to as novel coronavirus 2019, or 2019-nCoV). The disease was first identified in December 2019 and spread globally, causing a pandemic. Symptoms of COVID-19 include fever, cough, shortness of breath, fatigue, headache, loss of smell, nasal congestion, sore throat, coughing up sputum, pain in muscles or joints, chills, nausea, vomiting, and diarrhea. In severe cases, symptoms can include difficulty waking, confusion, blueish face or lips, coughing up blood, decreased white blood cell count, and kidney failure. Complications can include pneumonia, viral sepsis, acute respiratory distress syndrome, and kidney failure.

COVID-19 is especially threatening to public health. The virus is highly contagious, and studies currently indicate that it can be spread by asymptomatic carriers or by those who are pre-symptomatic. Likewise, the early stage of the disease is slow-progressing enough that carriers do not often realize they are infected, leading them to expose numerous others to the virus. The combination of COVID-19's ease of transmission, its high rate of hospitalization of victims, and its death rate make the virus a substantial public health risk, especially for countries without a healthcare system equipped to provide supportive care to pandemic-level numbers of patients. There is not yet a vaccine or specific antiviral treatment for COVID-19 and accordingly, there is a pressing need for treatments or cures.

SARS-CoV-2 is not the only coronavirus that causes disease. It is a β-coronavirus, a genus of coronaviruses that includes other human pathogens, including SARS-CoV (the causative agent of SARS), MERS-CoV (the causative agent of MERS), and HCoV-OC43 (a causative agent of the common cold). The infectivity of these viruses, and the severity of the diseases they cause, varies widely. β-coronavirus can also manifest as zoonotic infections, spread to and from humans and animals. Additionally, non-human species such as camels, bats, tigers, non-human primates, and rabbits can be susceptible to β-coronavirus. Accordingly, there is a pressing need for treatments or cures to multiple coronaviruses.

The present disclosure provides molecules useful against coronaviruses, and especially SARS-CoV-2, the causative agent of COVID-19 in humans. Accordingly, the present disclosure fulfills the need in the art for compounds that can be safely and effectively treat or prevent coronavirus infections in humans.

Picornaviruses are a family of positive strand RNA, nonenveloped viruses. A picornavirus has 60 identical subunits (vertices) which contain five protomers. Each protomer is made up of one copy of four proteins, named VP1, VP2, VP3 and VP4. There are several genera of picornaviruses, including, Enterovirus, Aphthovirus, Cardiovirus and Hepatovirus. Enteroviruses known to infect human include, but are not limited to, Rhinovirus A, Rhinovirus B, Rhinovirus C, Coxsackievirus A, Coxsackievirus B and Poliovirus. There is no specific treatment for a picornavirus infection.

Noroviruses are single-stranded positive-sense RNA, non-enveloped viruses belonging to the Caliciviridae family. Noroviruses are often spread by the fecal-oral route and are a common cause of gastroenteritis. Infected subjects can experience nausea, non-bloody diarrhea, vomiting and/or abdominal pain. Those suffering from a norovirus infection can become severely dehydrated and require medical attention. As with a picornavirus infection, there is no specific treatment for a norovirus infection. Accordingly, there is a need for compounds that effectively treat or prevent a picornavirus and/or a norovirus infection.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) (such as 1, 2 or 3) individually and independently selected from deuterium, alkyl, alkenyl, alkynyl, cycloalkyl, cycloalkenyl, aryl, heteroaryl, heterocyclyl, aryl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, C-amido(alkyl), isocyanato, thiocyanato, nitro, azido, silyl, sulfenyl, sulfinyl, sulfonyl, haloalkyl, haloalkoxy, trihalomethanesulfonyl, trihalomethanesulfonamido, an amino, a mono-substituted amine and a di-substituted amine.

As used herein, “Cₐ to C_{b}” or “C_{a-b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring of the cycloalkyl, ring of the cycloalkenyl, ring of the aryl, ring of the heteroaryl or ring of the heterocyclyl can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. The length of an alkenyl can vary. For example, the alkenyl can be a C₂₋₄ alkenyl, C₂₋₆ alkenyl or C₂₋₈ alkenyl. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. The length of an alkynyl can vary. For example, the alkynyl can be a C₂₋₄ alkynyl, C₂₋₆ alkynyl or C₂₋₈ alkynyl. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused- or spiro-fashion. Cycloalkyl groups can contain 3 to 10 atoms in the ring(s). 3 to 8 atoms in the ring(s) or 3 to 6 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused- or spiro-fashion. A cycloalkenyl can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to a monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The number of atoms in the ring(s) of a heterocyclyl group can vary. For example, the heterocyclyl group can contain 4 to 14 atoms in the ring(s), 5 to 10 atoms in the ring(s) or 5 to 6 atoms in the ring(s). The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl groups may be unsubstituted or substituted. Examples of such “heterocyclyl groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidinone, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and 3,4-methylenedioxyphenyl).

As used herein, “cycloalkyl(alkyl)” refers to a cycloalkyl group connected, as a substituent, via a lower alkylene group. The lower alkylene and cycloalkyl group of a cycloalkyl(alkyl) may be substituted or unsubstituted. A cycloalkyl(alkyl) group may be unsubstituted or substituted.

As used herein, “aryl(alkyl)” refers to an aryl group connected, as a substituent, via a lower alkylene group. The lower alkylene and aryl group of an aryl(alkyl) may be substituted or unsubstituted. Examples include but are not limited to benzyl, 2-phenyl(alkyl), 3-phenyl(alkyl), and naphthyl(alkyl).

As used herein, “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl), and their benzo-fused analogs.

A “heterocyclyl(alkyl)” refer to a heterocyclic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group with a substituent(s) listed under the definition of “substituted.” Further, when a lower alkylene group is substituted, the lower alkylene can be substituted by replacing both hydrogens on the same carbon with a cycloalkyl group

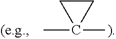

As used herein, “alkoxy” refers to the formula —OR wherein R is an alkyl, an alkenyl, an alkynyl, a cycloalkyl, a cycloalkenyl, an aryl, a heteroaryl, a heterocyclyl, a cycloalkyl(alkyl), an aryl(alkyl), a heteroaryl(alkyl) or a heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzyloxy. In some instances, an alkoxy can be —OR, wherein R is an unsubstituted C₁₋₄ alkyl. An alkoxy may be substituted or unsubstituted.

As used herein, “acyl” refers to a hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl) connected, as substituents, via a carbonyl group. Examples include formyl, acetyl, propanoyl, benzoyl and acryl. An acyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to a O-alkyl group and O-monocyclic cycloalkyl group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy, 2-fluoroisobutoxy, chloro-substituted cyclopropoxy, fluoro-substituted cyclopropoxy, chloro-substituted cyclobutoxy and fluoro-substituted cyclobutoxy. In some instances, a haloalkoxy can be —OR, wherein R is a C₁₋₄ alkyl substituted by 1, 2 or 3 halogens. A haloalkoxy may be substituted or unsubstituted.

A “sulfenyl” group refers to an “—SR” group in which R can be hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “—S(═O)₂R” group in which R can be the same as defined with respect to sulfenyl. A sulfonyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CS(═O)₂—” group wherein each X is a halogen.

A “trihalomethanesulfonamido” group refers to an “X₃CS(═O)₂N(R_{A})—” group wherein each X is a halogen, and R_{A} is hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—SCN” group.

An “isothiocyanato” group refers to an “—NCS” group.

A “mercapto” group refers to an “—SH” group.

A “carbonyl” group refers to a —C(═O)— group.

An “S-sulfonamido” group refers to a “—S(═O)₂N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An S-sulfonamido may be substituted or unsubstituted.

An “N-sulfonamido” group refers to a “RS(═O)₂N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-carbamyl may be substituted or unsubstituted.

An “O-thiocarbamyl” group refers to a “—OC(═S)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-amido may be substituted or unsubstituted.

A “mono-substituted amine” refers to a “—NHR_{A}” in which R_{A} can be independently an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A mono-substituted amine may be substituted or unsubstituted. In some instances, a mono-substituted amine can be —NHR_{A}, wherein R_{A} can be an unsubstituted C₁₋₆ alkyl or an unsubstituted or a substituted benzyl.

A “di-substituted amine” refers to a “—NR_{A}R_{B}” in which R_{A} and R_{B} can be independently can be independently an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A mono-substituted amine may be substituted or unsubstituted. In some instances, a mono-substituted amine can be —NR_{A}R_{B}, wherein R_{A} and R_{B} can be independently an unsubstituted C₁₋₆ alkyl or an unsubstituted or a substituted benzyl.

A “ketoamide” group refers to a —C(═O)—C(═O)N(R_{A}R_{B}) group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A ketoamide may be substituted or unsubstituted.

The term “halogen atom” or “halogen” as used herein, means any one of the radio-stable atoms of column 7 of the Periodic Table of the Elements, such as, fluorine, chlorine, bromine and iodine.

As used herein, the term “fused” refers to two rings which have two atoms and one bond in common. As used herein, the term “spiro” refers to two rings which have one atom in common and the two rings are not linked by a bridge.

Where the numbers of substituents are not specified (e.g., haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof. In addition, the term “comprising” is to be interpreted synonymously with the phrases “having at least” or “including at least”. When used in the context of a compound or composition, the term “comprising” means that the compound or composition includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality.

It is understood that, in any compound described herein having one or more chiral centers, if an absolute stereochemistry is not expressly indicated, then each center may independently be of (R)-configuration or (S)-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

### Compounds

Some embodiments disclosed herein relate to a compound of Formula (I), or a pharmaceutically acceptable salt thereof:

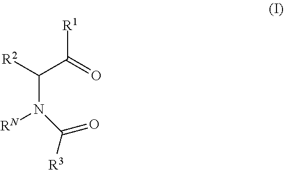

wherein: R^{N} can be hydrogen, deuterium or an unsubstituted or a substituted C₁₋₆ alkyl; R¹ can be

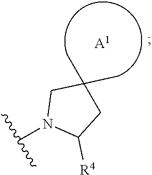

wherein Ring A¹ can be a 5-7 membered monocyclic heterocyclyl:
i) includes NR^{5a} in the ring;
ii) can be substituted with a first ═O on a carbon of the ring;
iii) optionally includes 1-3 heteroatoms selected from O (oxygen), S (sulfur), S(═O)₂, N (nitrogen) and NR^{5b} in the ring of Ring A¹;
iv) can be optionally substituted with one or more moieties selected from a second ═O on a ring carbon, halogen, hydroxy, an unsubstituted C₁₋₆ alkyl, an unsubstituted —O(C₁₋₆ alkyl), an unsubstituted or a substituted phenoxy, an unsubstituted or a substituted C₃₋₆ cycloalkyl, an unsubstituted or a substituted phenyl and an unsubstituted or a substituted benzyl;
v) can be optionally fused to an unsubstituted or a substituted phenyl, an unsubstituted or a substituted monocyclic heteroaryl, an unsubstituted or a substituted monocyclic heterocyclyl, an unsubstituted or a substituted bicyclic heteroaryl, an unsubstituted or a substituted bicyclic heterocyclyl, an unsubstituted or a substituted monocyclic cycloalkenyl or an unsubstituted or a substituted bicyclic cycloalkenyl; and
vi) provided that when R¹ is

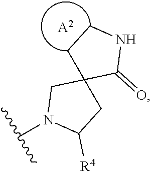

then Ring A² can be an unsubstituted or a substituted monocyclic heterocyclyl, an unsubstituted or a substituted monocyclic cycloalkenyl or an unsubstituted or a substituted bicyclic cycloalkenyl;
R² can be hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₁₀ cycloalkyl, an unsubstituted or a substituted C₃₋₁₀ cycloalkenyl, an unsubstituted or a substituted aryl, an unsubstituted or a substituted aryl(alkyl), an unsubstituted or a substituted heteroaryl, an unsubstituted or a substituted heteroaryl(alkyl), an unsubstituted or a substituted heterocyclyl or an unsubstituted or a substituted heterocyclyl(alkyl); R³ can be

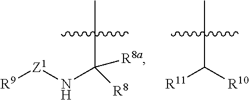

or R¹²; Z¹ can be —C(═O)— or —S(═O)₂—; R⁴ can be selected from cyano, an unsubstituted or a substituted C₂₋₅ alkynyl, an unsubstituted or a substituted acyl, an unsubstituted or a substituted ketoamide, —C(═O)NH₂, —CH(OH)—(S(═O)₂—OH), —CH(OH)—(S(═O)₂—O—), —CH(OH)((P═O)(OR⁶)₂) and —C(═O)CH₂—O—((P═O)(OR⁷)₂); R^{5a} can be selected from hydrogen, an unsubstituted or a substituted C₁₋₄ alkyl, an unsubstituted or a substituted C₂₋₄ alkenyl and an unsubstituted or a substituted C₃₋₆ cycloalkyl; R^{5b} can be selected from hydrogen, an unsubstituted or a substituted C₁₋₄ alkyl, an unsubstituted or a substituted C₂₋₄ alkenyl and an unsubstituted or a substituted C₃₋₆ cycloalkyl; each R⁶ and each R⁷ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl); R⁸ and R¹⁰ can be independently selected from an unsubstituted or a substituted C₁₋₆ alkyl, an unsubstituted or a substituted C₂₋₆ alkenyl, an unsubstituted or a substituted C₂₋₆ alkynyl, an unsubstituted or a substituted monocyclic C₃₋₆ cycloalkyl, an unsubstituted or a substituted bicyclic C₅₋₈ cycloalkyl and an unsubstituted or a substituted monocyclic 4- to 6-membered heterocyclyl, wherein when the C₁₋₆ alkyl is substituted, the C₁₋₆ alkyl can be substituted 1, 2, 3 or 4 times with a substituent independently selected from halogen, cyano, —NH₂, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic 5- or 6-membered heteroaryl, an unsubstituted or a substituted monocyclic 4-6 membered heterocyclyl, an unsubstituted C₁₋₄ alkoxy, an unsubstituted or a substituted phenoxy, an unsubstituted or a substituted —O—(CH₂)-phenyl and an unsubstituted C₁₋₄ haloalkoxy, or the C₁₋₆ alkyl is substituted 1 to 13 times with deuterium; wherein when the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted, the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl can be substituted 1, 2, 3 or 4 times with a substituent independently selected from halogen, an unsubstituted C₁₋₄ alkyl, an unsubstituted C₂₋₄ alkenyl, an unsubstituted C₂₋₄ alkynyl, an unsubstituted C₁₋₄ haloalkyl, an unsubstituted or a substituted monocyclic C₃₋₆ cycloalkyl and an unsubstituted C₁₋₄ alkoxy; and R^{8a} can be hydrogen or an unsubstituted C₁₋₄ alkyl; or R⁸ and R^{8a} can be taken together to form an unsubstituted monocyclic C₃₋₆ cycloalkyl or a halogen-substituted monocyclic C₃₋₆ cycloalkyl; R⁹ can be selected from an unsubstituted or a substituted C₁₋₆ alkyl, an unsubstituted or a substituted C₁₋₆ haloalkyl, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic heteroaryl, an unsubstituted or a substituted monocyclic heterocyclyl, an unsubstituted or a substituted alkoxy (such as —O—(C₁₋₄ alkyl), —O—(C₃₋₆ cycloalkyl) and —O-(phenyl), wherein the C₃₋₆ cycloalkyl and phenyl can be unsubstituted or substituted (for example substituted 1, 2, 3 or 4 times with halogen, an unsubstituted C₁₋₄ alkyl, an unsubstituted C₁₋₄ alkoxy and/or an unsubstituted C₁₋₄ haloalkyl) and —NR¹⁷R¹⁸, wherein the substituted C₁₋₆ alkyl can be substituted 1 or 2 times with a substituent selected from hydroxy and an unsubstituted C₁₋₄ alkoxy, wherein the substituted monocyclic C₃₋₆ cycloalkyl can be substituted 1, 2, 3 or 4 times with a substituent independently selected from halogen, an unsubstituted C₁₋₄ alkyl, an unsubstituted C₁₋₄ alkoxy, an unsubstituted C₁₋₄ haloalkyl and an unsubstituted monocyclic C₃₋₆ cycloalkyl, and wherein the substituted C₁₋₆ haloalkyl can be substituted 1 or 2 times with an unsubstituted C₁₋₄ alkoxy; R″ can be an unsubstituted or a substituted monocyclic 4- to 6-membered heterocyclyl, —(NH)ₘ-(an unsubstituted or a substituted 5- to 10-membered heteroaryl), —O-(an unsubstituted or a substituted C₁₋₆ alkyl), —O-(an unsubstituted or a substituted C₃₋₈ cycloalkyl) or —O—(C₁₋₄ alkyl)-(an unsubstituted or a substituted C₃₋₈ cycloalkyl), wherein m can be 1; R¹² can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted 5- to 8-membered bicyclic heterocyclyl, an unsubstituted or a substituted aryl(alkyl), an unsubstituted or a substituted heteroaryl(alkyl), an unsubstituted or a substituted heterocyclyl(alkyl), an unsubstituted or a substituted C-carboxy, —OR¹³, —NR¹⁴R¹⁵ or —C(═O)—NR^{16A}R^{16B}; R¹³ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl); R¹⁴ are R¹⁵ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) and an unsubstituted or a substituted heteroaryl(alkyl); R^{16A} can be hydrogen or an unsubstituted C₁₋₃ alkyl; R^{16B} can be an unsubstituted or a substituted aryl, an unsubstituted or a substituted heteroaryl or an unsubstituted or a substituted 3- to 8-membered monocyclic heterocyclyl; and R¹⁷ and R¹⁸ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl); or R¹⁷ and R¹⁸ can be taken together along with the nitrogen to which they are connected to form an unsubstituted or a substituted 3-8 membered heterocyclyl.

In some embodiments, R¹ can be

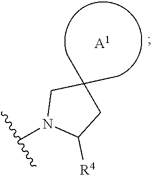

wherein Ring A¹ can be a 5-7 membered monocyclic heterocyclyl that: i) can include NR^{5a} in the ring; ii) can be substituted with a first ═O on a carbon of the ring; iii) can optionally include 1-3 heteroatoms selected from O (oxygen), S (sulfur), S(═O)₂, N (nitrogen) and NR^{5b} in the ring of Ring A¹; iv) can be optionally substituted with one or more moieties selected from a second ═O on a ring carbon, halogen, hydroxy, an unsubstituted C₁₋₆ alkyl, an unsubstituted —O(C₁₋₆ alkyl), an unsubstituted or a substituted phenoxy, an unsubstituted or a substituted C₃₋₆ cycloalkyl, an unsubstituted or a substituted phenyl and an unsubstituted or a substituted benzyl; and v) can be optionally fused to an unsubstituted or a substituted phenyl, an unsubstituted or a substituted monocyclic heteroaryl, an unsubstituted or a substituted monocyclic heterocyclyl, an unsubstituted or a substituted bicyclic heteroaryl, an unsubstituted or a substituted bicyclic heterocyclyl, an unsubstituted or a substituted monocyclic cycloalkenyl or an unsubstituted or a substituted bicyclic cycloalkenyl. In some embodiments, Ring A¹ can be a 5-membered monocyclic heterocyclyl. In other embodiments, Ring A¹ can be a 6-membered monocyclic heterocyclyl. In still other embodiments, Ring A¹ can be a 7-membered monocyclic heterocyclyl.

In some embodiments, the NR^{5a} in the 5-7 membered monocyclic heterocyclyl of Ring A¹ can be adjacent to the ring carbon that is substituted with a first ═O. In some embodiments, the carbon substituted by a first ═O of the 5-7 membered monocyclic heterocyclyl of Ring A¹ can be adjacent to the spiro-connect point (indicated with an asterisk

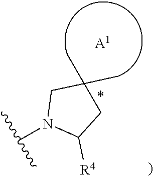

of R¹. In some embodiments, the carbon substituted by a first ═O of the 5-7 membered monocyclic heterocyclyl of Ring A¹ can be adjacent to the spiro-connect point (indicated with an asterisk

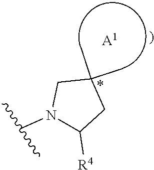

of R¹; and the NR^{5a} in the 5-7 membered monocyclic heterocyclyl of Ring A¹ can be adjacent to the ring carbon that is substituted with a first ═O.

In some embodiments, R^{5a} can be hydrogen. As provided herein, R^{5a} can be a non-hydrogen moiety. For example, R^{5a} can be an unsubstituted or a substituted C₁₋₄ alkyl, an unsubstituted or a substituted C₂₋₄ alkenyl and an unsubstituted or a substituted C₃₋₆ cycloalkyl. In some embodiments, R^{5a} can be an unsubstituted C₁₋₄ alkyl. In other embodiments, R^{5a} can be a substituted C₁₋₄ alkyl. In still other embodiments, R^{5a} can be an unsubstituted C₂₋₄ alkenyl. In yet still other embodiments, R^{5a} can be a substituted C₂₋₄ alkenyl. In some embodiments, R^{5a} can be an unsubstituted C₃₋₆ cycloalkyl. In other embodiments, R^{5a} can be a substituted C₃₋₆ cycloalkyl. For example, R^{5a} can be an unsubstituted or a substituted monocyclic C₃₋₆ cycloalkyl. In some embodiments, R^{5a} can be methyl. In some embodiments, R^{5a} can be cyclopropyl.

In some embodiments, the 5-7 membered monocyclic heterocyclyl of Ring A¹ can include 1-3 heteroatoms selected from O (oxygen), S (sulfur), N (nitrogen) and NR^{5b} in the ring of Ring A¹. For example, the 5-7 membered monocyclic heterocyclyl of Ring A¹ can include O (oxygen), the 5-7 membered monocyclic heterocyclyl of Ring A¹ can include S (sulfur), the 5-7 membered monocyclic heterocyclyl of Ring A¹ can include S(═O)₂, the 5-7 membered monocyclic heterocyclyl of Ring A¹ can include N (nitrogen) and/or the 5-7 membered monocyclic heterocyclyl of Ring A¹ can include NR^{5b}. In some embodiments, Ring A¹ can be a 5-membered monocyclic heterocyclyl that includes N (nitrogen). In some embodiments, Ring A¹ can be a 5-membered monocyclic heterocyclyl that includes NR^{5b}. In some embodiments, Ring A¹ can be a 5-membered monocyclic heterocyclyl that includes NR^{5b} and a second ═O on a ring carbon. In some embodiments, Ring A¹ can be a 6-membered monocyclic heterocyclyl that includes N (nitrogen). In some embodiments, Ring A¹ can be a 6-membered monocyclic heterocyclyl that includes O (oxygen). In other embodiments, Ring A¹ can be a 7-membered monocyclic heterocyclyl that includes O (oxygen).

In some embodiments of this previous paragraph, R^{5a} can be hydrogen. As provided herein, R^{5a} can be a non-hydrogen moiety. For example, R^{5a} can be an unsubstituted or a substituted C₁₋₄ alkyl, an unsubstituted or a substituted C₂₋₄ alkenyl and an unsubstituted or a substituted C₃₋₆ cycloalkyl. In some embodiments of this previous paragraph, R^{5a} can be an unsubstituted C₁₋₄ alkyl. In other embodiments of this previous paragraph, R^{5a} can be a substituted C₁₋₄ alkyl. In still other embodiments of this previous paragraph, R^{5a} can be an unsubstituted C₂₋₄ alkenyl. In yet still other embodiments of this previous paragraph, R^{5a} can be a substituted C₂₋₄ alkenyl. In some embodiments of this previous paragraph, R^{5a} can be an unsubstituted C₃₋₆ cycloalkyl. In other embodiments of this previous paragraph, R^{5a} can be a substituted C₃₋₆ cycloalkyl. For example, R^{5a} can be an unsubstituted or a substituted monocyclic C₃₋₆ cycloalkyl. In some embodiments, R^{5a} can be methyl. In some embodiments, R^{5a} can be cyclopropyl.

As provided herein, Ring A¹ can be optionally substituted with one or more moieties (such as 1, 2 or 3 moieties) selected from a second ═O on ring carbon, halogen, hydroxy, an unsubstituted C₁₋₆ alkyl, an unsubstituted —O(C₁₋₆ alkyl), an unsubstituted or a substituted phenoxy, an unsubstituted or a substituted C₃₋₆ cycloalkyl, an unsubstituted or a substituted phenyl and an unsubstituted or a substituted benzyl. Exemplary groups that can be present on Ring A¹ can be selected from fluoro, chloro, hydroxy, methyl, ethyl, n-propyl, iso-propyl, n-butyl, iso-butyl, sec-butyl, tert-butyl, pentyl (straight-chained and/or branched), hexyl (straight-chained and/or branched), methoxy, ethoxy, n-propoxy, iso-propoxy, n-butoxy, iso-butoxy, sec-butoxy, tert-butoxy, pentoxy (straight-chained and/or branched), hexoxy (straight-chained and/or branched), an unsubstituted or a substituted phenoxy, an unsubstituted or a substituted C₃₋₆ cycloalkyl, an unsubstituted or a substituted phenyl and an unsubstituted or a substituted benzyl. In some embodiments, the unsubstituted or a substituted C₃₋₆ cycloalkyl that can be present on Ring A¹ can be an unsubstituted or a substituted monocyclic C₃₋₆ cycloalkyl, such as cyclopropyl, cyclobutyl, cyclopentyl and cyclohexyl. In some embodiments, the unsubstituted or a substituted C₃₋₆ cycloalkyl that can be present on Ring A¹ can be an unsubstituted or a substituted bicyclic C₃₋₆ cycloalkyl. For example, when Ring A¹ is substituted with an unsubstituted or a substituted bicyclic C₃₋₆ cycloalkyl, the unsubstituted or a substituted bicyclic C₃₋₆ cycloalkyl can be an unsubstituted or a substituted spiro[2.2]pentane, an unsubstituted or a substituted spiro[2.3]hexane, an unsubstituted or a substituted bicyclo[1.1.1]pentane or an unsubstituted or a substituted bicyclo[2.1.1]hexane. The monocyclic C₃₋₆ cycloalkyl and bicyclic C₃₋₆ cycloalkyl that can be substituted on Ring A¹ can be connected via 1 ring carbon of the C₃₋₆ cycloalkyl (for example,

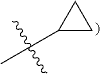 [SMILES: *C1CC1]

or a fused-fashion via 2 ring carbons of the C₃₋₆ cycloalkyl (for example

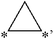

wherein the asterisks indicate the points of attachment) or spiro-fashion via 1 ring carbon of the C₃₋₆ cycloalkyl (for example,

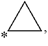

wherein the asterisk indicates the point of attachment). An example of a C₃₋₆ cycloalkyl spiro-connected to Ring A¹ is compound 113 shown below.

In some embodiments, Ring A¹ can be substituted with an unsubstituted phenyl. In other embodiments, Ring A¹ can be substituted with a substituted phenyl, such as a mono-substituted phenyl, a di-substituted phenyl or a phenyl substituted with 3 to 5 substituents. A non-limiting list of moieties that can be present on substituted phenyl that is substituted on Ring A¹ include halogen (such as bromo, chloro and fluoro), cyano, an unsubstituted C₁₋₄ alkyl (for example, methyl, ethyl, n-propyl, iso-propyl, n-butyl, iso-butyl, sec-butyl and tert-butyl), hydroxy, an unsubstituted C₁₋₄ alkoxy (for example, —O(C₁₋₄ alkyl such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, CH(CH₃)F, —CH₂CF₃, —CH₂CH₂F and —CH₂CH₂CH₂F), an unsubstituted C₁₋₄ haloalkoxy (for example, —O(C₁₋₄ haloalkyl such as —OCF₃, —OCHF₂, —OC(CH₃)F₂, —OCH₂F, —OCH(CH₃)F, —OCH₂CF₃, —OCH₂CH₂F and —OCH₂CH₂CH₂F), an unsubstituted monocyclic C₃₋₆ cycloalkyl, a substituted monocyclic C₃₋₆ cycloalkyl, an unsubstituted phenyl, a substituted phenyl, an unsubstituted 5- or 6-membered heteroaryl and a substituted 5- or 6-membered heteroaryl (for example, a substituted monocyclic C₃₋₆ cycloalkyl, a substituted phenyl and/or a substituted 5- or 6-membered heteroaryl can be substituted 1, 2, 3, 4 or 5 times with a substituent selected from halogen (for example, F, Cl and Br), an unsubstituted C₁₋₄ alkyl (for example, methyl, ethyl, n-propyl, iso-propyl, n-butyl, iso-butyl, sec-butyl and tert-butyl), an unsubstituted C₁₋₄ alkoxy (for example —O(C₁₋₄ alkyl such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted —O(an unsubstituted C₁₋₄ haloalkyl) (for example, —OCF₃, —OCHF₂, —OC(CH₃)F₂, —OCH₂F, —OCH(CH₃)F, —OCH₂CF₃, —OCH₂CH₂F and —OCH₂CH₂CH₂F) and —S(═O)₂(an unsubstituted C₁₋₄ alkyl.

In some embodiments, Ring A¹ can be fused to an unsubstituted or a substituted phenyl. In other embodiments, Ring A¹ can be fused to an unsubstituted or a substituted monocyclic heteroaryl. For example, the unsubstituted or a substituted monocyclic heteroaryl can be a 5- or 6-membered unsubstituted or a substituted monocyclic heteroaryl that can include 1, 2, 3 or 4 heteroatoms selected from O (oxygen), N (nitrogen) and S (sulfur). A non-limiting list of monocyclic heteroaryls that can be fused to Ring A¹ include furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine and pyrazine. When Ring A¹ includes N and is fused to an unsubstituted or a substituted heteroaryl, the nitrogen can be positioned at a fusion-point of the two rings (Ring A¹ and the unsubstituted or a substituted heteroaryl). Exemplary rings that can be fused to Ring A¹ include the following:

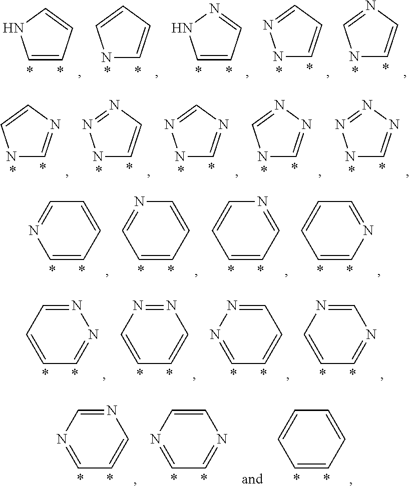

wherein the asterisks indicates the point of attachment to Ring A¹ and each can be unsubstituted or substituted (including the replacement of the hydrogen on the nitrogen(s)).

In some embodiments, Ring A¹ can be fused to an unsubstituted or a substituted monocyclic heterocyclyl. As an example, Ring A¹ can be fused to an unsubstituted or a substituted 5- or 6-membered monocyclic heterocyclyl that can include 1, 2, 3 or 4 heteroatoms selected from O (oxygen), N (nitrogen) and S (sulfur). A non-limiting list of monocyclic heterocyclyls that can be fused to Ring A¹ are provided herein and include, but not limited to, pyrrolidine, pyrrolidinone (such as pyrrolidin-2-one and pyrrolidin-3-one), piperidine, piperazine, morpholine, thiomorpholine, tetrahydropyran, tetrahydrothiopyran, pyrazolidin-3-one, imidazolidin-4-one, imidazolidine-2,4-dione, 3,5-dihydro-4H-imidazol-4-one, tetrahydropyrimidin-4(1H)-one, tetrahydropyridazin-3(2H)-one, piperazin-2-one, 4,5-dihydropyridazin-3(2H)-one, morpholin-3-one, thiomorpholin-3-one, thiomorpholin-3-one 1,1-dioxide, 1,3-oxazinan-2-one and 1,4-oxazepan-3-one.

In still other embodiments, Ring A¹ can be fused to an unsubstituted or a substituted bicyclic heteroaryl. In yet still other embodiments, Ring A¹ can be fused to an unsubstituted or a substituted bicyclic heterocyclyl. For example, Ring A¹ can be fused to an unsubstituted or a substituted 9- or 10-membered bicyclic heteroaryl, such as 5,6-unsubstituted or a substituted bicyclic heteroaryl or 6,6-unsubstituted or a substituted bicyclic heteroaryl, or an unsubstituted or a substituted 9- or 10-membered bicyclic heterocyclyl, such as 5,6-unsubstituted or a substituted bicyclic heterocyclyl or 6,6-unsubstituted or a substituted bicyclic heterocyclyl. The unsubstituted or a substituted bicyclic heteroaryl and/or unsubstituted or a substituted bicyclic heterocyclyl can include 1, 2, 3 or 4 heteroatoms, for example, O (oxygen), N (nitrogen) and/or S (sulfur). Examples of an unsubstituted or a substituted bicyclic heteroaryl that can be fused to Ring A¹ include indazolyl, isoindole, quinolinyl, isoquinolinyl, phthalazinyl, quinazolinyl, quinoxalinyl, 1,8-naphthyridinyl, 1,7-naphthyridinyl, 1,6-naphthyridinyl, 1,5-naphthyridinyl, 2,6-naphthyridinyl and 2,7-naphthyridinyl. Examples of an unsubstituted or a substituted bicyclic heterocyclyl that can be fused to Ring A¹ are 4,5,6,7-tetrahydro-2H-indazolyl, imidazo[1,2-α]pyridinyl, imidazo[1,5-α]pyridinyl, pyrazolo[1,5-α]pyridinyl, oxazolo[3,2-α]pyridinyl, thiazolo[3,2-α]pyridinyl, isoxazolo[2,3-α]pyridinyl and isothiazolo[2,3-α]pyridinyl.

In some embodiments, Ring A¹ can be fused to an unsubstituted or a substituted monocyclic cycloalkenyl. In other embodiments, Ring A¹ can be fused to an unsubstituted or a substituted bicyclic cycloalkenyl. For example, Ring A¹ can be fused to an unsubstituted or a substituted monocyclic C₅₋₆ cycloalkenyl or an unsubstituted or a substituted bicyclo[4.2.0]octa-1(6),2,4-triene.

The phenyl, the monocyclic heteroaryl, the bicyclic heteroaryl, the monocyclic heterocyclyl, the bicyclic heterocyclyl, the monocyclic cycloalkenyl and the bicyclic cycloalkenyl that is fused to Ring A¹ can be substituted. For example, the phenyl, the monocyclic heteroaryl, the bicyclic heteroaryl, the monocyclic heterocyclyl, the bicyclic heterocyclyl, the monocyclic cycloalkenyl and the bicyclic cycloalkenyl that is fused to Ring A¹ can be substituted with one or more moieties (such as 1, 2 or 3 moieties). Examples of suitable moieties that can be present on a phenyl, a monocyclic heteroaryl, a bicyclic heteroaryl, a monocyclic heterocyclyl, a bicyclic heterocyclyl. a monocyclic cycloalkenyl and/or a bicyclic cycloalkenyl can be independently selected from halogen (such as bromo, chloro and fluoro), cyano, an unsubstituted C₁₋₄ alkyl (for example, methyl, ethyl, n-propyl, iso-propyl, n-butyl, iso-butyl, sec-butyl and tert-butyl), hydroxy, an unsubstituted C₁₋₄ alkoxy (for example —O(an unsubstituted C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted C₁₋₄ haloalkoxy (for example, —O(an unsubstituted C₁₋₄ haloalkyl), such as —OCF₃, —OCHF₂, —OC(CH₃)F₂, —OCH₂F, —OCH(CH₃)F, —OCH₂CF₃, —OCH₂CH₂F and —OCH₂CH₂CH₂F), —S(═O)₂(an unsubstituted C₁₋₄ alkyl), —C(═O)—NR^{N1}R^{N2} wherein R^{N1} and R^{N2} are independently hydrogen or an unsubstituted C₁₋₄ alkyl or R^{N1} and R^{N2} are taken together to form a monocyclic heterocyclyl (such as a pyrrolidinyl, piperidinyl and morpholinyl), an unsubstituted monocyclic C₃₋₆ cycloalkyl, a substituted monocyclic C₃₋₆ cycloalkyl, an unsubstituted phenyl, a substituted phenyl, an unsubstituted 5- or 6-membered heteroaryl and a substituted 5- or 6-membered heteroaryl (wherein, for example, a substituted monocyclic C₃₋₆ cycloalkyl, a substituted phenyl and/or a substituted 5- or 6-membered heteroaryl can be substituted 1, 2, 3, 4 or 5 times with a substituent independently selected from halogen (for example, F, Cl and Br), cyano, an unsubstituted C₁₋₄ alkyl (for example, methyl, ethyl, n-propyl, iso-propyl, n-butyl, iso-butyl, sec-butyl and tert-butyl), an unsubstituted C₁₋₄ alkoxy (e.g., —O(an unsubstituted C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted —O(an unsubstituted C₁₋₄ haloalkyl) (for example, —OCF₃, —OCHF₂, —OC(CH₃)F₂, —OCH₂F, —OCH(CH₃)F, —OCH₂CF₃, —OCH₂CH₂F and —OCH₂CH₂CH₂F) and —S(═O)₂(an unsubstituted C₁₋₄ alkyl). In some embodiments, the phenyl, the monocyclic heteroaryl, the bicyclic heteroaryl, the monocyclic heterocyclyl, the bicyclic heterocyclyl, the monocyclic cycloalkenyl and the bicyclic cycloalkenyl that is fused to Ring A¹ can be substituted at a carbon next to the fusion point. As example is shown in this structure where the carbon next to the fusion point is indicated with a “+”

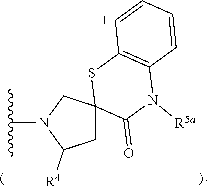

Examples of Ring A¹ moieties include pyrrolidinone (such as pyrrolidin-2-one and pyrrolidin-3-one), piperidine, piperazine, morpholine, thiomorpholine, tetrahydropyran, tetrahydrothiopyran, pyrazolidin-3-one, imidazolidinone (for example, imidazolidin-4-one), imidazolidine-2,4-dione, 3,5-dihydro-4H-imidazol-4-one, tetrahydropyridazin-3(2H)-one, tetrahydropyrimidinone (such as tetrahydropyrimidin-4(1H)-one), piperazin-2-one, morpholin-3-one, 4,5-dihydropyridazin-3(2H)-one, thiomorpholin-3-one, thiomorpholin-3-one 1,1-dioxide, 1,3-oxazinan-2-one and 1,4-oxazepan-3-one. In some embodiments, Ring A¹ can be selected from:

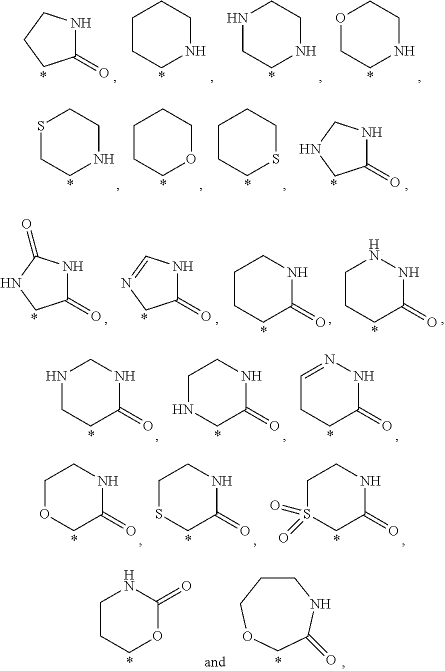

wherein the asterisk indicates the point of attachment to

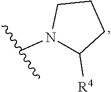

and each can be unsubstituted or substituted.

As provided herein, R¹ can be

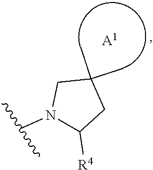

wherein Ring^{A} can be a 5-7 membered monocyclic heterocyclyl, and Ring A¹ can be optionally fused to an unsubstituted or a substituted phenyl, an unsubstituted or a substituted monocyclic heteroaryl, an unsubstituted or a substituted monocyclic heterocyclyl, an unsubstituted or a substituted bicyclic heteroaryl, an unsubstituted or a substituted bicyclic heterocyclyl, an unsubstituted or a substituted monocyclic cycloalkenyl or an unsubstituted or a substituted bicyclic cycloalkenyl. In some embodiments, when R¹ is

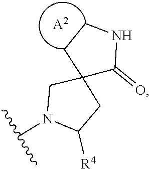

then Ring A² can be an unsubstituted or a substituted monocyclic heterocyclyl, an unsubstituted or a substituted monocyclic cycloalkenyl or an unsubstituted or a substituted bicyclic cycloalkenyl. In some embodiments, when R¹ is

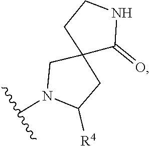

then Ring A¹ cannot be fused to another ring, such as an unsubstituted or a substituted phenyl, an unsubstituted or a substituted monocyclic heteroaryl, an unsubstituted or a substituted monocyclic heterocyclyl, an unsubstituted or a substituted bicyclic heteroaryl, an unsubstituted or a substituted bicyclic heterocyclyl, an unsubstituted or a substituted monocyclic cycloalkenyl or an unsubstituted or a substituted bicyclic cycloalkenyl).

In some embodiments, R¹ can be

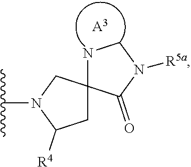

wherein Ring A³ can be absent, such that the nitrogen shown connected to Ring A³ is NH or NR^{5b}. In other embodiments, R¹ can be

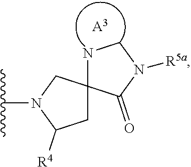

wherein Ring A³ can be an unsubstituted or a substituted monocyclic heteroaryl, an unsubstituted or a substituted monocyclic heterocyclyl, an unsubstituted or a substituted bicyclic heteroaryl or an unsubstituted or a substituted bicyclic heterocyclyl.

In some embodiments, R¹ can be R

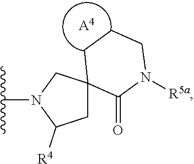

wherein Ring A⁴ can be absent. In other embodiments, R¹ can be

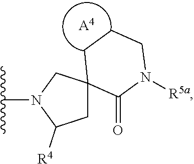

wherein Ring A⁴ can be an unsubstituted or a substituted phenyl, an unsubstituted or a substituted monocyclic cycloalkenyl, an unsubstituted or a substituted monocyclic heteroaryl, an unsubstituted or a substituted monocyclic heterocyclyl, an unsubstituted or a substituted bicyclic cycloalkenyl, an unsubstituted or a substituted bicyclic heteroaryl or an unsubstituted or a substituted bicyclic heterocyclyl.

In some embodiments, R¹ can be

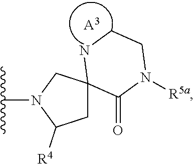

wherein Ring A³ can be absent, such that the nitrogen shown connected to Ring A³ is NH or NR^{5b}. In other embodiments, R¹ can be

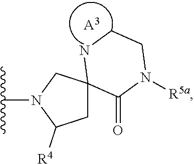

wherein Ring A³ can be an unsubstituted or a substituted monocyclic heteroaryl, an unsubstituted or a substituted bicyclic heteroaryl, an unsubstituted or a substituted monocyclic heterocyclyl or an unsubstituted or a substituted bicyclic heterocyclyl.

In some embodiments, R¹ can be

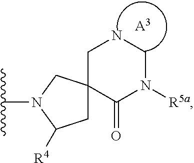

wherein Ring A³ can be absent, such that the nitrogen shown connected to Ring A³ is NH or NR^{5b}. In other embodiments, R¹ can be

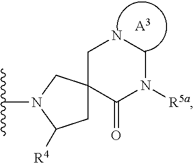

wherein Ring A³ can be an unsubstituted or a substituted monocyclic heteroaryl, an unsubstituted or a substituted monocyclic heterocyclyl, an unsubstituted or a substituted bicyclic heteroaryl or an unsubstituted or a substituted bicyclic heterocyclyl.

In some embodiments, R¹ can be

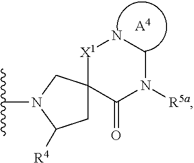

wherein Ring A⁴ can be absent. In other embodiments, R¹ can be

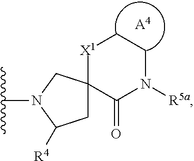

wherein Ring A⁴ can be an unsubstituted or a substituted phenyl, an unsubstituted or a substituted monocyclic cycloalkenyl, an unsubstituted or a substituted monocyclic heteroaryl, an unsubstituted or a substituted monocyclic heterocyclyl, an unsubstituted or a substituted bicyclic cycloalkenyl, an unsubstituted or a substituted bicyclic heteroaryl or an unsubstituted or a substituted bicyclic heterocyclyl; and X¹ can be —CR^{y1}R^{Y2}—, —CR^{Y3}R^{Y4}CR^{Y5}R^{Y6}—, —O—, —S—, S(═O)₂, NR^{5b} or —OCH₂—, wherein R^{y1}, R^{Y2}, R^{Y3}, R^{Y4}, R^{Y5} and R^{Y6} can be independently selected from hydrogen, halogen, hydroxy and an unsubstituted C₁₋₆ alkyl. In some embodiments, R^{y1} and R^{Y2} can be each hydrogen. In some embodiments, R^{Y3}, R^{Y4}, R^{Y5} and R^{Y6} can be each hydrogen. In other embodiments, one of R^{y1} and R^{Y2} can be halogen, hydroxy or an unsubstituted C₁₋₆ alkyl; and the other one of R^{y1} and R^{Y2} can be hydrogen. In still other embodiments, R^{y1} and R^{Y2} can be each halogen, such as F. In some embodiments, one of R^{Y3}, R^{Y4}, R^{Y5} and R^{Y6} can be halogen, hydroxy or an unsubstituted C₁₋₆ alkyl; and the remaining of RY³, RY⁴, RY⁵ and R^{Y6} can be hydrogen. In other embodiments, two of RY³, RY⁴, RY⁵ and R^{Y6} can be halogen, hydroxy or an unsubstituted C₁₋₆ alkyl; and the remaining of R^{Y3}, R^{Y4}, R^{Y5} and R^{Y6} can be hydrogen.

In some embodiments, R¹ can be R

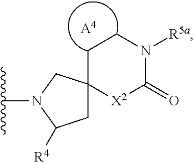

Ring A⁴ can be absent. In other embodiments, R¹ can be

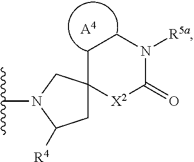

Ring A⁴ can be an unsubstituted or a substituted phenyl, an unsubstituted or a substituted monocyclic cycloalkenyl, an unsubstituted or a substituted monocyclic heteroaryl, an unsubstituted or a substituted monocyclic heterocyclyl, an unsubstituted or a substituted bicyclic cycloalkenyl, an unsubstituted or a substituted bicyclic heteroaryl or an unsubstituted or a substituted bicyclic heterocyclyl; and X² can be —CH₂—, —CH₂CH₂—, —O—, —S—, —S(═O)₂— or —OCH₂—.

In some embodiments, R¹ can be

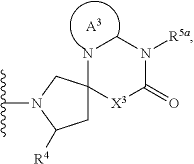

Ring A³ can be absent, such that the nitrogen shown connected to Ring A³ is NH or NR^{5b}. In other embodiments, R¹ can be

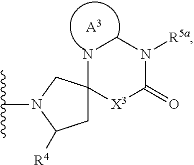

Ring A³ can be an unsubstituted or a substituted monocyclic heteroaryl, an unsubstituted or a substituted monocyclic heterocyclyl, an unsubstituted or a substituted bicyclic heteroaryl or an unsubstituted or a substituted bicyclic heterocyclyl; and X³ can be —CH₂—, —CH₂CH₂—, —O—, —S—, —S(═O)₂—, NR^{5b} or —OCH₂—.

In some embodiments, R¹ can be

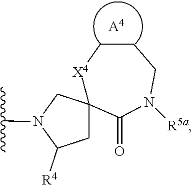

Ring A⁴ can be absent. In other embodiments, R¹ can be

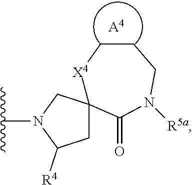

Ring A⁴ can be an unsubstituted or a substituted phenyl, an unsubstituted or a substituted monocyclic cycloalkenyl, an unsubstituted or a substituted monocyclic heteroaryl, an unsubstituted or a substituted monocyclic heterocyclyl, an unsubstituted or a substituted bicyclic cycloalkenyl, an unsubstituted or a substituted bicyclic heteroaryl or an unsubstituted or a substituted bicyclic heterocyclyl; and X⁴ can be —CH₂—, —O—, —S—, —S(═O)₂— or NR^{5b}. In some embodiments, R¹ can be

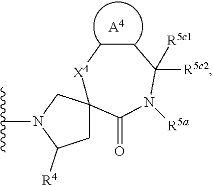

wherein R^{5c1} and R^{5c2} can be independently hydrogen, halogen or an unsubstituted C₁₋₄ alkyl; or R^{5c1} and R^{5c2} can be taken together along with the carbon to which R^{5c1} and R^{5c2} are attached to form an unsubstituted or a substituted C₃₋₆ cycloalkyl, such as those described herein. In some embodiments, at least one of R^{5c1} and R^{5c2} can be halogen or an unsubstituted C₁₋₄ alkyl. In other embodiments, R^{5c1} and R^{5c2} can be both halogen, both an unsubstituted C₁₋₄ alkyl or R^{5c1} and R^{5c2} can be taken together along with the carbon to which R^{5c1} and R^{5c2} are attached to form an unsubstituted or a substituted C₃₋₆ cycloalkyl.

In some embodiments, R¹ can be

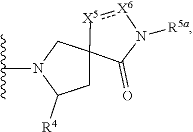

wherein [symbol] can be a single bond; X⁵ can be —CR^{Z1a}R^{Z1b}—, —(CR^{Z2a}R^{Z2b})(CR^{Z3a}R^{Z3b})—, —O—, —S—, —O(CR^{Z4a}R^{Z4b})—, —S(CR^{Z5a}R^{Z5b})—, —O(CR^{Z6a}R^{Z6b})(CR^{Z7a}R^{Z7b})—, —S(CR^{Z8a}R^{Z8b})(CR^{Z9a}R^{Z9b})— or NR^{5b}; X⁶ can be —CR^{Z10a}R^{Z10b}—, —C(═O)— or NR^{5b}; provided that X⁵ and X⁶ cannot be both NR⁵b; R^{Z1a}, R^{Z1b}, R^{Z4a}, R^{Z4b}, R^{Z5a}, R^{Z5b}, R^{Z6a}, R^{Z6b}, R^{Z7a}, R^{Z7b}, R^{Z8a}, R^{Z8b}, R^{Z9a} and R^{Z9b} can be independently selected from hydrogen, halogen, hydroxy, an unsubstituted C₁₋₆ alkyl, an unsubstituted —O(C₁₋₆ alkyl), an unsubstituted or a substituted C₃₋₆ cycloalkyl (such as an unsubstituted or a substituted monocyclic C₃₋₆ cycloalkyl), an unsubstituted or a substituted phenyl, an unsubstituted or a substituted phenoxy, and an unsubstituted or a substituted benzyl; R^{Z10a} and R^{Z10b} can be independently selected from hydrogen, halogen, hydroxy, an unsubstituted C₁₋₆ alkyl, an unsubstituted —O(C₁₋₆ alkyl), an unsubstituted or a substituted C₃₋₆ cycloalkyl (such as an unsubstituted or a substituted monocyclic C₃₋₆ cycloalkyl), an unsubstituted or a substituted phenyl, an unsubstituted or a substituted phenoxy, and an unsubstituted or a substituted benzyl; or R^{Z10a} and R^{Z10b} can be taken together along with the carbon to which R^{Z10a} and R^{Z10b} are attached to form an unsubstituted or a substituted spiro-connected C₃₋₆ cycloalkyl; and R^{Z2a}, R^{Z2b}, R^{Z3a} and R^{Z3b} can be independently selected from hydrogen, halogen, hydroxy, an unsubstituted C₁₋₆ alkyl, an unsubstituted or a substituted C₃₋₆ cycloalkyl, an unsubstituted or a substituted phenyl and an unsubstituted or a substituted benzyl, or R^{Z2a} and R^{Z3a} can be taken together along with the carbons to which each is attached to form an unsubstituted or a substituted monocyclic cycloalkenyl or an unsubstituted or a substituted bicyclic cycloalkenyl; or wherein [symbol] can be a double bond; X⁵ can be CR^{Z11} or CR^{Z12a}R^{Z12b}CR^{Z13}; X⁶ can be N or CR^{Z14}; and R^{Z11}, R^{Z12a}, R^{Z12b}, R^{Z13} and R^{Z14} can be independently selected from hydrogen, halogen, hydroxy, an unsubstituted C₁₋₆ alkyl, an unsubstituted or a substituted C₃₋₆ cycloalkyl (such as an unsubstituted or a substituted monocyclic C₃₋₆ cycloalkyl), an unsubstituted or a substituted phenyl and an unsubstituted or a substituted benzyl. In other embodiments, R¹ can be

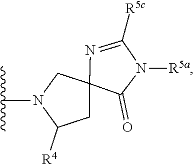

wherein R^{5C} can be selected from hydrogen, halogen, hydroxy, an unsubstituted C₁₋₆ alkyl, an unsubstituted or a substituted C₃₋₆ cycloalkyl (such as an unsubstituted or a substituted monocyclic C₃₋₆ cycloalkyl), an unsubstituted or a substituted phenyl and an unsubstituted or a substituted benzyl. In some embodiments of this paragraph, Ring A⁴ can be an unsubstituted or a substituted phenyl. In some embodiments of this paragraph, Ring A³ can be a 5-membered unsubstituted or substituted monocyclic heteroaryl. In other embodiments of this paragraph, Ring A³ can be a 6-membered unsubstituted or substituted monocyclic heteroaryl. In some embodiments of this paragraph, Ring A⁴ can be a 5-membered unsubstituted or substituted monocyclic heteroaryl. In other embodiments of this paragraph, Ring A⁴ can be a 6-membered unsubstituted or substituted monocyclic heteroaryl. In some embodiments, the unsubstituted or a substituted monocyclic heteroaryl for Ring A³ and/or Ring A⁴ can include 1, 2, 3 or 4 nitrogens in the ring. When Ring A³ and/or Ring A⁴ is substituted, Ring A³ and/or Ring A⁴ can be substituted 1, 2, 3 or 4 times with moieties independently selected from halogen (F, C₁ or Br), cyano, an unsubstituted C₁₋₄ alkyl (for example, methyl, ethyl, n-propyl, iso-propyl, n-butyl, iso-butyl, sec-butyl and tert-butyl), hydroxy, an unsubstituted C₁₋₄ alkoxy (for example —O(an unsubstituted C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted C₁₋₄ haloalkoxy (for example —O(an unsubstituted C₁₋₄ haloalkyl) such as —OCF₃, —OCHF₂, —OC(CH₃)F₂, —OCH₂F, —OCH(CH₃)F, —OCH₂CF₃, —OCH₂CH₂F and —OCH₂CH₂CH₂F), —S(═O)₂(an unsubstituted C₁₋₄ alkyl), —C(═O)—NR^{N1}R^{N2}, wherein R^{N1} and R^{N2} are independently hydrogen or an unsubstituted C₁₋₄ alkyl or R^{N1} and R^{N2} are taken together to form a monocyclic heterocyclyl (such as a pyrrolidinyl, piperidinyl and morpholinyl), an unsubstituted monocyclic C₃₋₆ cycloalkyl, a substituted monocyclic C₃₋₆ cycloalkyl, an unsubstituted phenyl, a substituted phenyl, an unsubstituted 5- or 6-membered heteroaryl and a substituted 5- or 6-membered heteroaryl (wherein, for example, a substituted monocyclic C₃₋₆ cycloalkyl, a substituted phenyl and/or a substituted 5- or 6-membered heteroaryl can be substituted 1, 2, 3, 4 or 5 times with a substituent selected from halogen (for example, F, Cl and Br), an unsubstituted C₁₋₄ alkyl (for example, methyl, ethyl, n-propyl, iso-propyl, n-butyl, iso-butyl, sec-butyl and tert-butyl), an unsubstituted C₁₋₄ alkoxy (e.g. —O(an unsubstituted C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted —O(an unsubstituted C₁₋₄ haloalkyl) (for example, —OCF₃, —OCHF₂, —OC(CH₃)F₂, —OCH₂F, —OCH(CH₃)F, —OCH₂CF₃, —OCH₂CH₂F and —OCH₂CH₂CH₂F)).

For

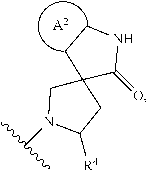

those skilled in the art understand that when Ring A² is an unsubstituted or a substituted monocyclic heterocyclyl, an unsubstituted or a substituted monocyclic cycloalkenyl or an unsubstituted or a substituted bicyclic cycloalkenyl, the fused bond between Ring A² and the pyrrolidin-2-one (Ring A¹) can be a single bond or a double bond depending on where a double bond of the unsubstituted or a substituted monocyclic heterocyclyl, the unsubstituted or a substituted monocyclic cycloalkenyl or the unsubstituted or a substituted bicyclic cycloalkenyl is positioned within Ring A². Similarly, for

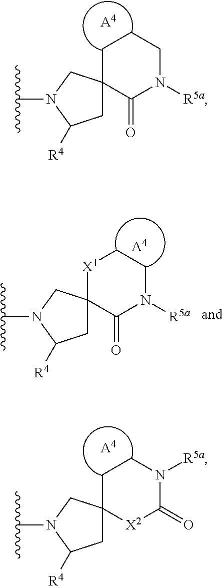

those skilled in the art understand that when Ring A⁴ is an unsubstituted or a substituted phenyl, an unsubstituted or a substituted monocyclic heteroaryl, an unsubstituted or a substituted monocyclic heterocyclyl, an unsubstituted or a substituted bicyclic heteroaryl, an unsubstituted or a substituted bicyclic heterocyclyl, an unsubstituted or a substituted monocyclic cycloalkenyl or an unsubstituted or a substituted bicyclic cycloalkenyl, the fused bond between Ring A¹ and Ring A⁴ can be a single bond or a double bond depending on where a double bond of the unsubstituted or a substituted phenyl, the unsubstituted or a substituted monocyclic heteroaryl, the unsubstituted or substituted monocyclic heterocyclyl, the unsubstituted or a substituted bicyclic heteroaryl, the unsubstituted or a substituted bicyclic heterocyclyl, the unsubstituted or substituted monocyclic cycloalkenyl or the unsubstituted or a substituted bicyclic cycloalkenyl is positioned within Ring A⁴.

Exemplary R¹ groups include the following:

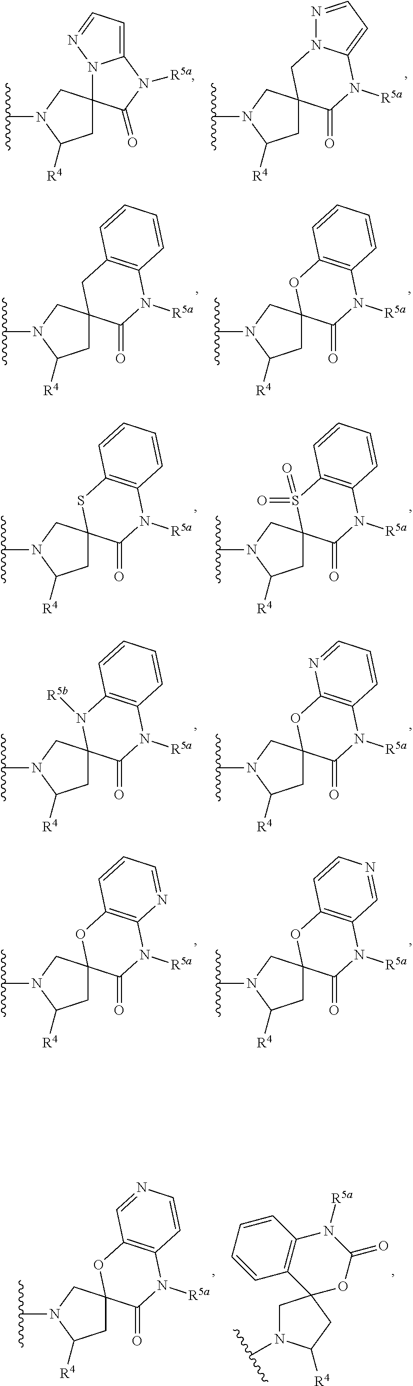

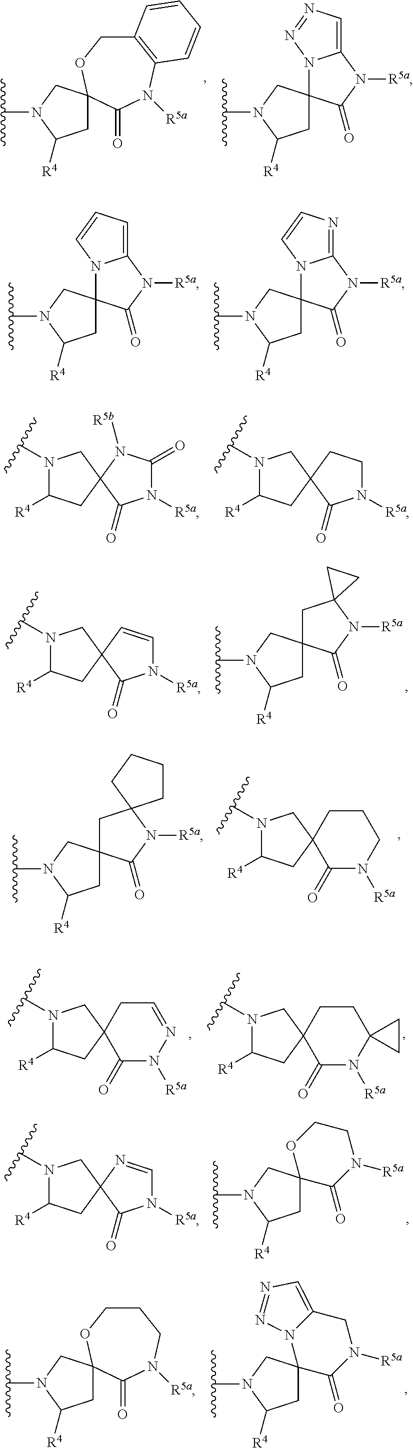

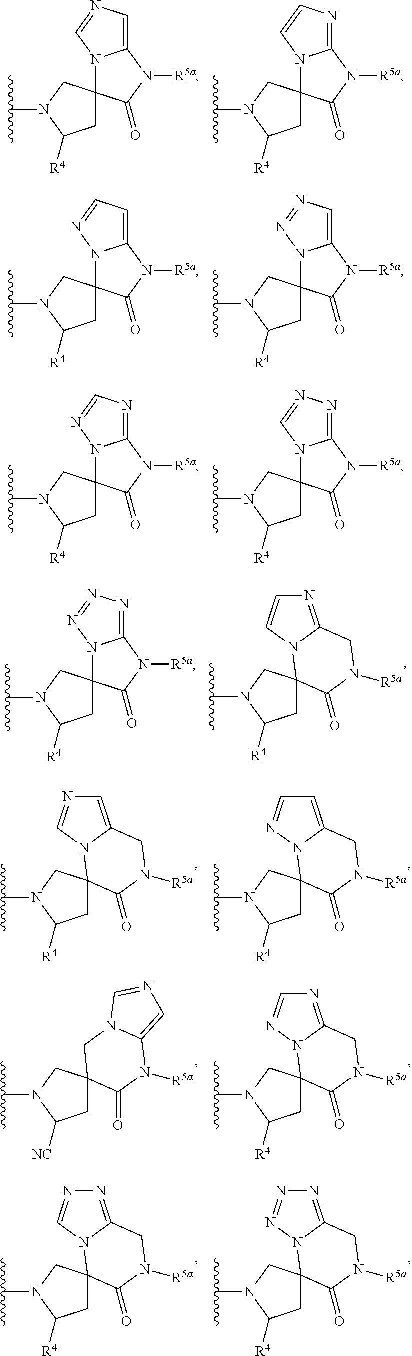

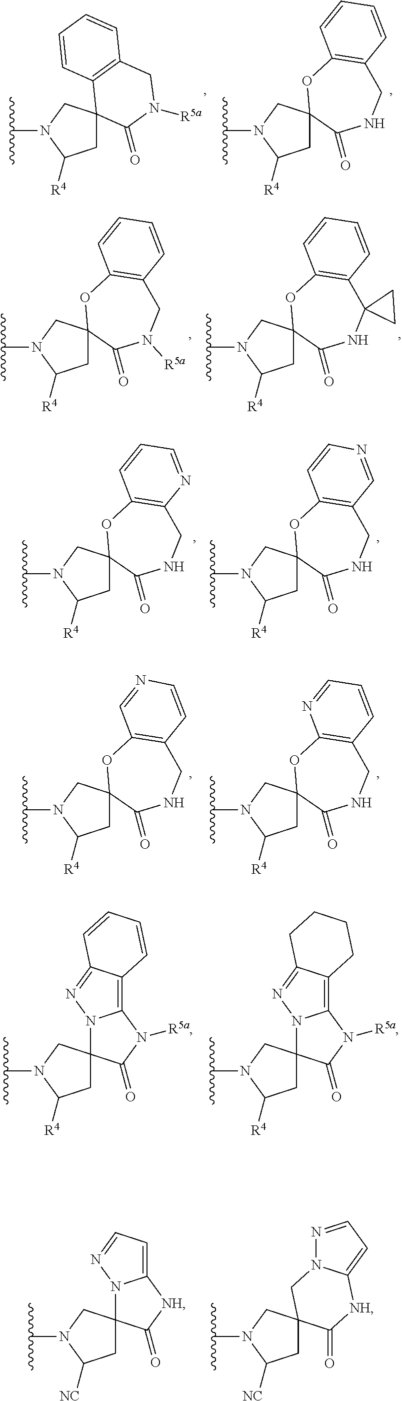

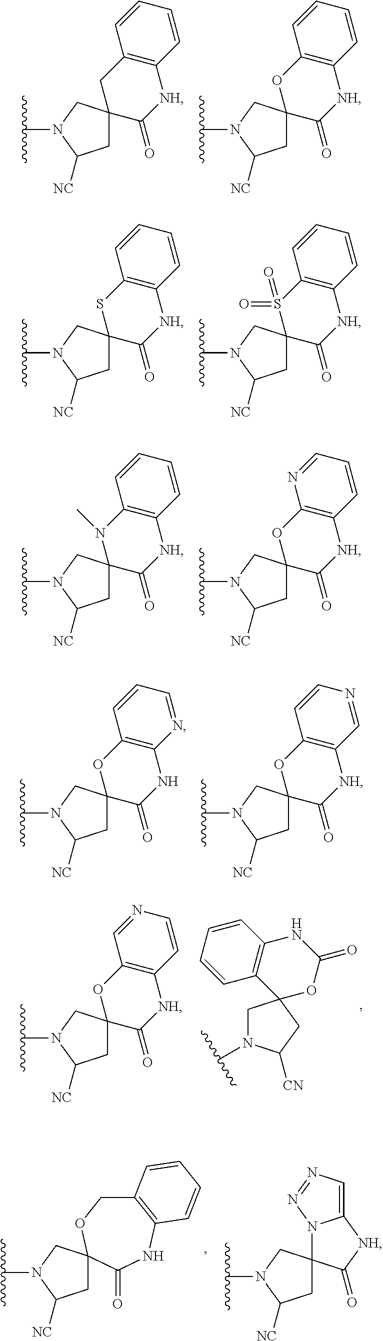

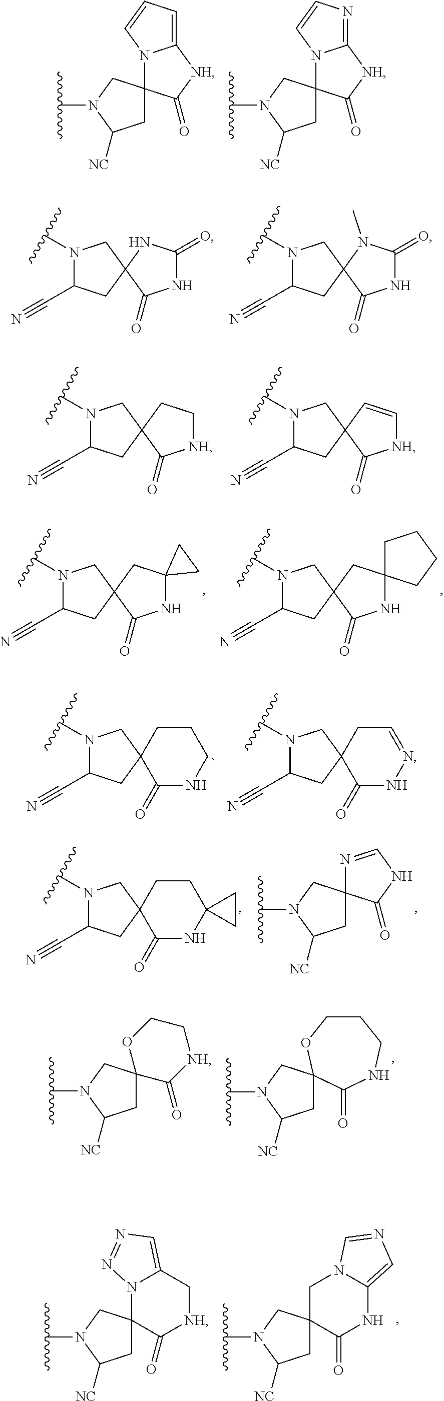

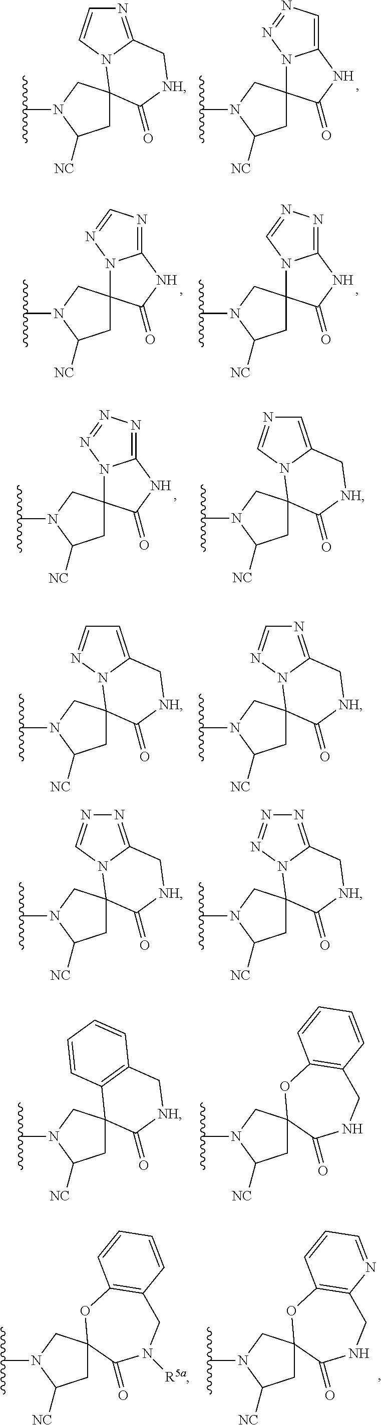

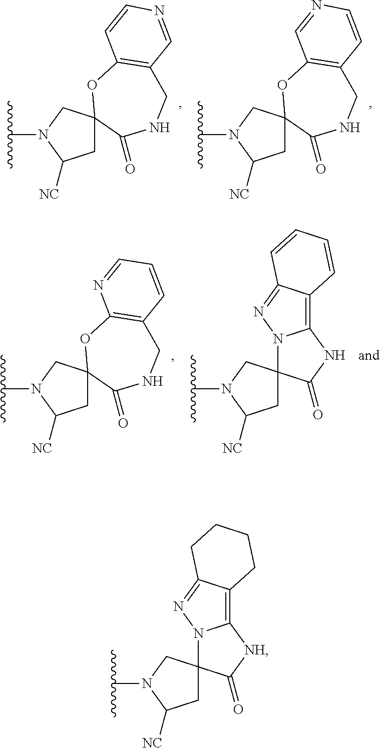

wherein each can be unsubstituted or substituted.

Further examples of R¹ groups include the following:

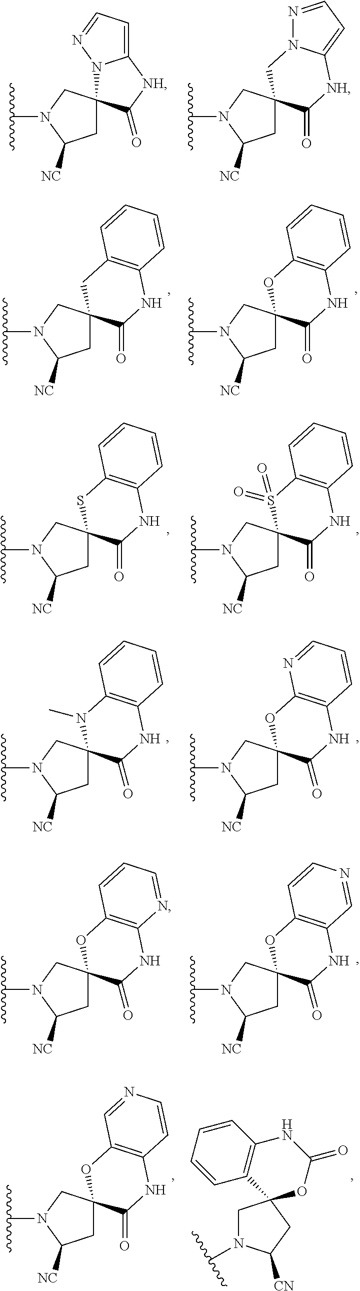

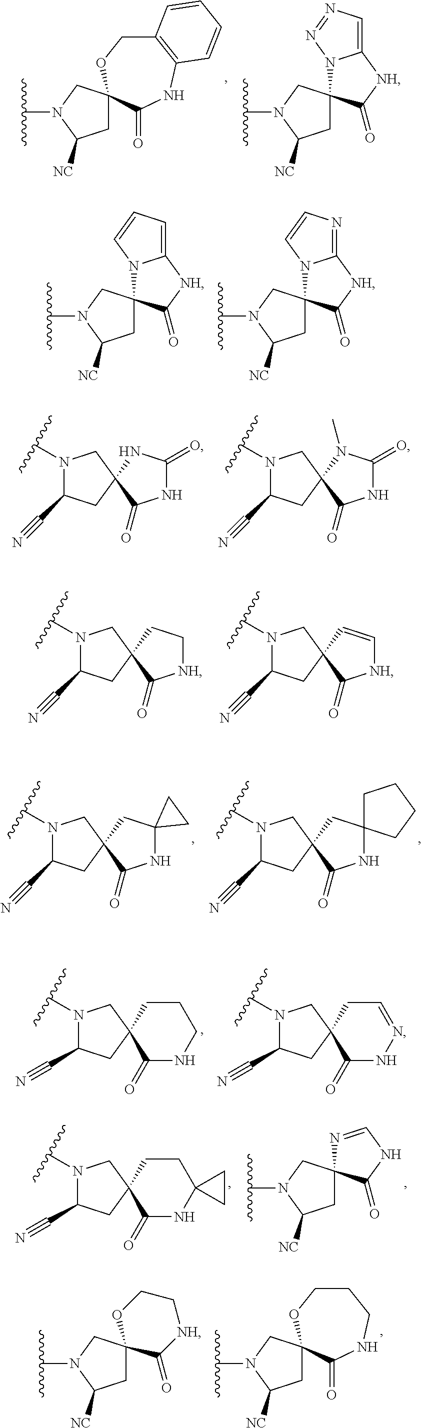

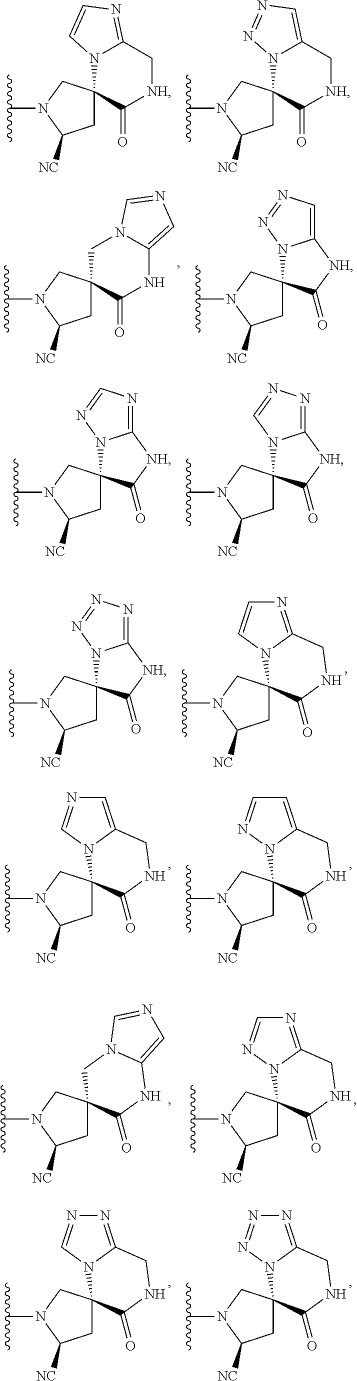

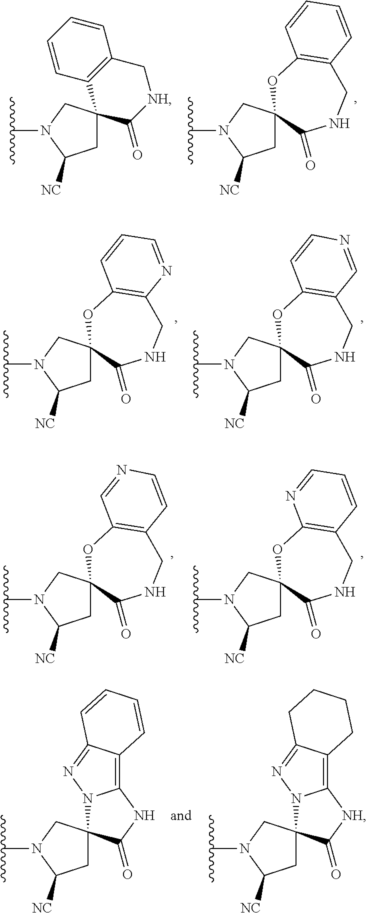

wherein each can be unsubstituted or substituted, including the replacement of the hydrogen on a nitrogen. Examples of substituted version include the following:

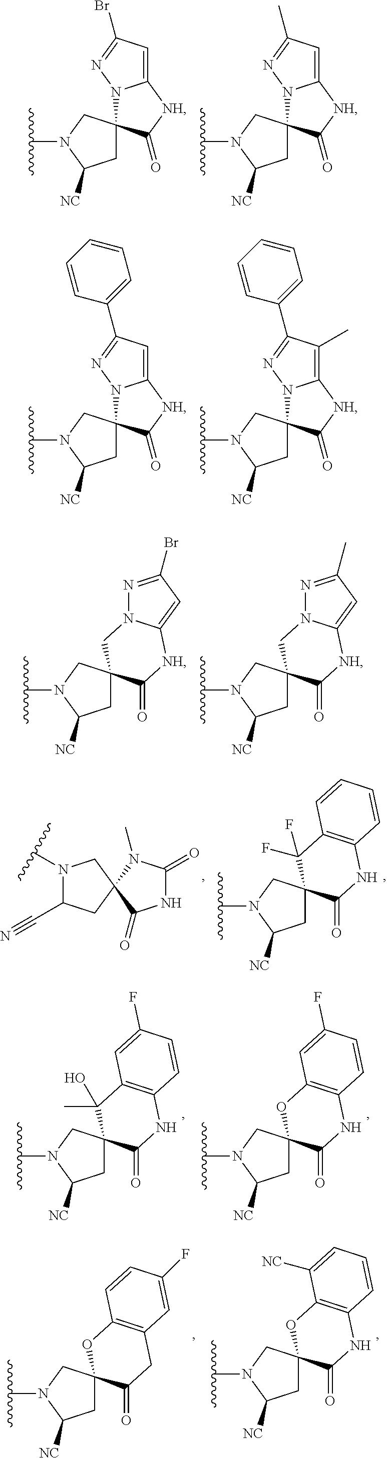

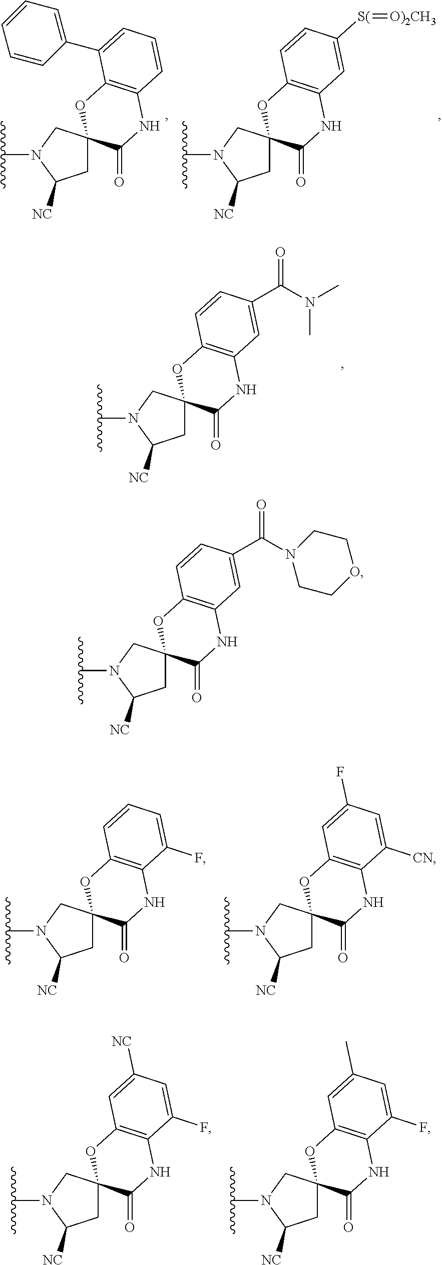

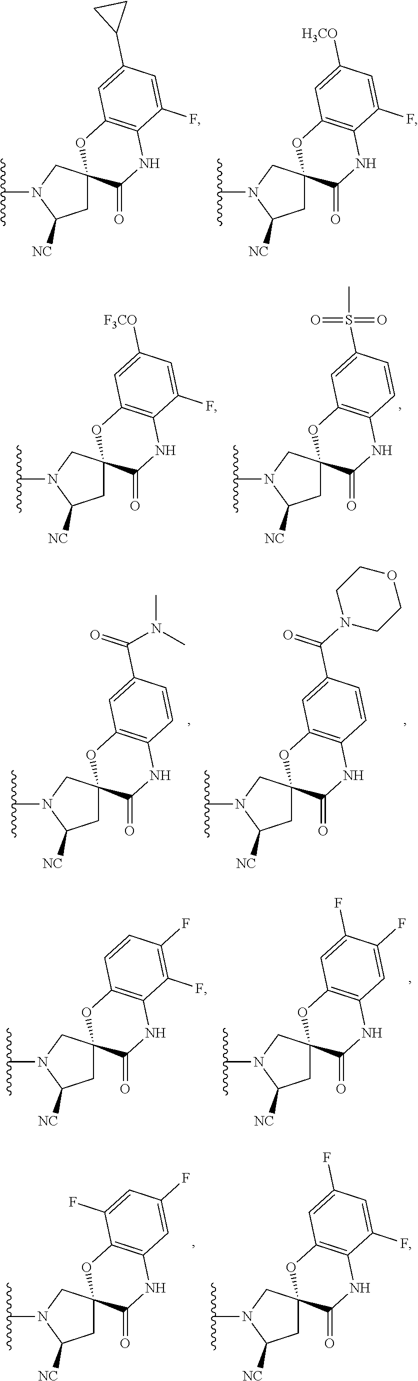

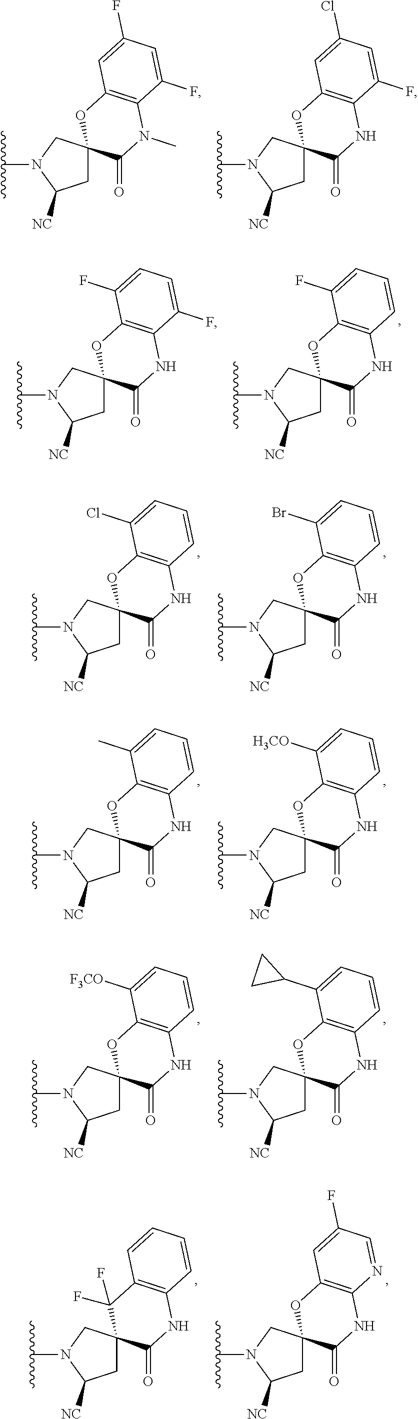

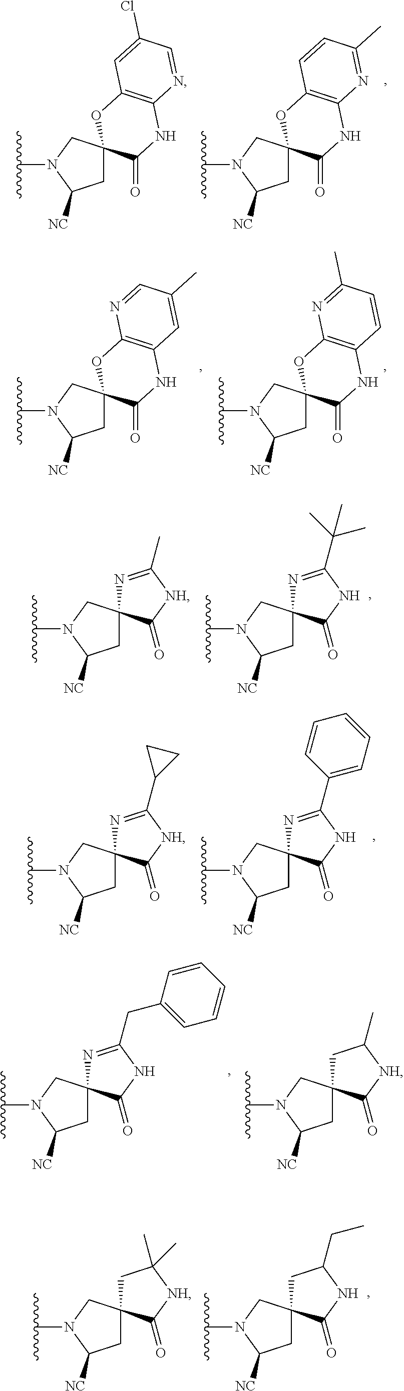

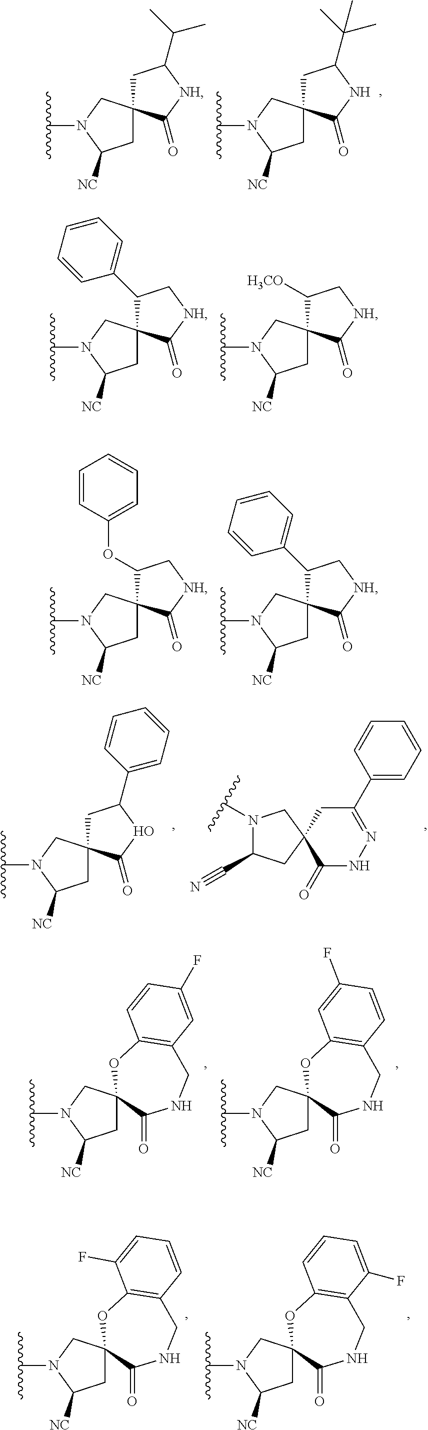

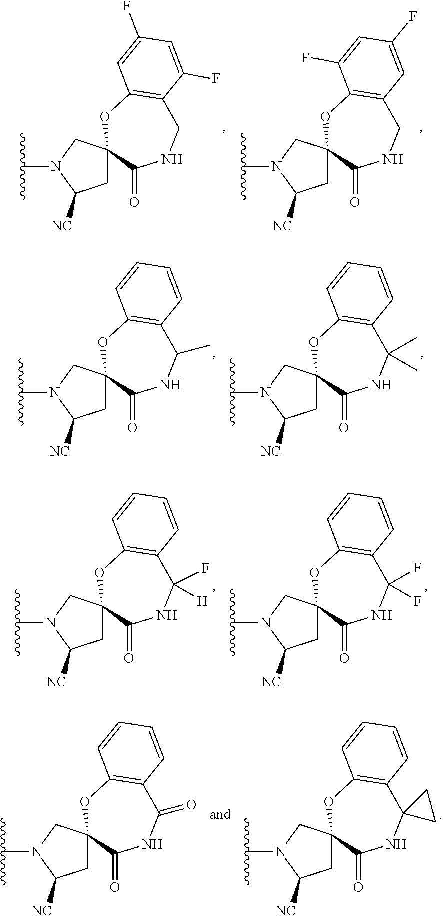

In some embodiments, R^{N} can be hydrogen. In other embodiments, R^{N} can be deuterium. In still other embodiments, R^{N} can be an unsubstituted C₁₋₆ alkyl. For example, R^{N} can be methyl, ethyl, n-propyl, iso-propyl, n-butyl, iso-butyl, sec-butyl, tert-butyl, pentyl (straight-chained and/or branched) or hexyl (straight-chained and/or branched). In some embodiments, R^{N} can be methyl. In yet still other embodiments, R^{N} can be a substituted C₁₋₆ alkyl. When R^{N} is a substituted C₁₋₆ alkyl, the C₁₋₆ alkyl can be substituted one or more times (such as 1, 2, 3, 4, 5 or 6 times) with substituent(s) independently selected from those provided for “optionally substituted.” In some embodiments, R^{N} can be a C₁₋₆ alkyl substituted 1, 2, 3, 4, 5 or 6 times with a halogen (such as bromo, chloro and/or fluoro). In some embodiments, R^{N} can be a C₁₋₆ alkyl substituted 1, 2, 3, 4, 5 or 6 times with deuterium.

In some embodiments, R² can be hydrogen. In other embodiments, R² can be an unsubstituted C₁₋₈ alkyl. For example, R² can be methyl, ethyl, n-propyl, iso-propyl, n-butyl, iso-butyl, sec-butyl, tert-butyl, pentyl (straight-chained and branched), hexyl (straight-chained and branched), heptyl (straight-chained and branched) and octyl (straight-chained and branched). In still other embodiments, R² can be a substituted C₁₋₈ alkyl. For example, R² can be a C₁₋₈ alkyl substituted with a monocyclic C₃₋₆ cycloalkyl (wherein the monocyclic C₃₋₆ cycloalkyl is unsubstituted or substituted), a C₁₋₈ alkyl substituted with one or more halogens (for example, 1, 2, 3, 4, 5 or 6 halogen) or a C₁₋₈ alkyl substituted with a —O— (an unsubstituted C₁₋₄ alkyl). The monocyclic C₃₋₆ cycloalkyl that can be substituted on a C₁₋₈ alkyl can be cyclopropyl, cyclobutyl, cyclopentyl or cyclohexyl. In some embodiments, R² can be —(CH₂)— (an unsubstituted or a substituted monocyclic C₃₋₆ cycloalkyl). When the C₃₋₆ cycloalkyl that is substituted on a C₁₋₈ alkyl is substituted, one or more other moieties can be present. For example, 1, 2, 3 or 4 moieties can be present on a C₃₋₆ cycloalkyl that is substituted on a C₁₋₈ alkyl for R². In some embodiments, R² can be a C₁₋₄ alkyl substituted with a cyclopropyl, cyclobutyl or a cyclohexyl (wherein the cyclopropyl, cyclobutyl and/or cyclohexyl can be unsubstituted or substituted with 1 or 2 halogens and/or 1 or 2 unsubstituted C₁₋₄ alkyls). In other embodiments, R² can be a C₁₋₄ alkyl substituted with 1, 2, 3, 4, 5 or 6 fluoros.

As described herein, R² can be an unsaturated hydrocarbon. In some embodiments, R² can be an unsubstituted C₂₋₈ alkenyl. In other embodiments, R² can be a substituted C₂₋₈ alkenyl. In still other embodiments, R² can be an unsubstituted C₂₋₈ alkynyl. In yet still other embodiments, R² can be a substituted C₂₋₈ alkynyl.

A variety of cyclic groups can be present for R² where the cyclic group can be a hydrocarbon cyclic group or a cyclic group that include 1, 2, 3 or 4 heteroatoms (such as N (nitrogen), O (oxygen) and S (sulfur)). In some embodiments, R² can be an unsubstituted C₃₋₁₀ cycloalkyl. In other embodiments, R² can be a substituted C₃₋₁₀ cycloalkyl. The C₃₋₁₀ cycloalkyl can be a monocyclic C₃₋₁₀ cycloalkyl or a multicyclic C₃₋₁₀ cycloalkyl (for example, a bicyclic or tricyclic C₃₋₁₀ cycloalkyl). In other embodiments, R² can be an unsubstituted C₃₋₁₀ cycloalkenyl. In still other embodiments, R² can be a substituted C₃₋₁₀ cycloalkenyl. As with the C₃₋₁₀ cycloalkyl, the C₃₋₁₀ cycloalkenyl can be monocyclic or multicyclic.

In some embodiments, R² can be an unsubstituted aryl. In other embodiments, R² can be a substituted aryl. In still other embodiments, R² can be an unsubstituted heteroaryl. In yet still other embodiments, R² can be a substituted heteroaryl. In some embodiments, R² can be an unsubstituted heterocyclyl. In other embodiments, R² can be a substituted heterocyclyl. The aryl, heteroaryl and heterocyclyl for R² can be monocyclic or multicyclic (for example, bicyclic or tricyclic).

The cyclic moiety that can be present for R² can be connected via an C₁₋₄ alkylene linker. For example, the cyclic moiety for R² can be connected via a methylene linker. In some embodiments, R² can be an unsubstituted aryl(alkyl). In other embodiments, R² can be a substituted aryl(alkyl). In still other embodiments, R² can be an unsubstituted heteroaryl(alkyl). In yet still other embodiments, R² can be a substituted heteroaryl(alkyl). In some embodiments, R² can be an unsubstituted heterocyclyl(alkyl). In other embodiments, R² can be a substituted heterocyclyl(alkyl).

As described herein, the cyclic moiety for R² can be monocyclic or multicyclic (for example, bicyclic or tricyclic). When R² includes a monocyclic aryl, R² can include an unsubstituted or a substituted phenyl. In some embodiments, R² can be an unsubstituted phenyl. In other embodiments, R² can be a substituted phenyl. In still other embodiments, R² can be an unsubstituted benzyl. In yet still other embodiments, R² can be a substituted benzyl. For example, the benzyl can be substituted with 1, 2, 3 or 4 halogens, such as fluoro and/or chloro, and/or cyano.

When R² includes a heteroaryl or a heterocyclyl (such as when R² is a heteroaryl, a heterocyclyl, a heteroaryl(alkyl) or a heterocyclyl(alkyl)), 1, 2 or 3 heteroatoms can be present in the ring(s). In some embodiments, the heterocyclyl that can be included in R² can be a 3- to 10-membered heterocyclyl. Examples of suitable heteroatoms include N (nitrogen), O (oxygen) and S (sulfur). In some embodiments, R² can include a monocyclic heteroaryl (such as a 5- or 6-membered heteroaryl). In other embodiments, R² can include a monocyclic heterocyclyl (such as a 5- or 6-membered heterocyclyl). In still other embodiments, R² can include a bicyclic heteroaryl (such as a 9- or 10-membered heteroaryl). In other embodiments, R² can include a bicyclic heterocyclyl (such as a 9- or 10-membered heterocyclyl). Examples or suitable heteroaryls and heterocyclyls include furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine, pyrazine, azetidine, oxetane, thietane, tetrahydrofuran, tetrahydrothiophene, pyrrolidine, oxazolidin-2-one, tetrahydropyran, tetrahydrothiopyran, piperidine, piperazine, morpholine and thiomorpholine.

A non-limiting list of R² groups include:

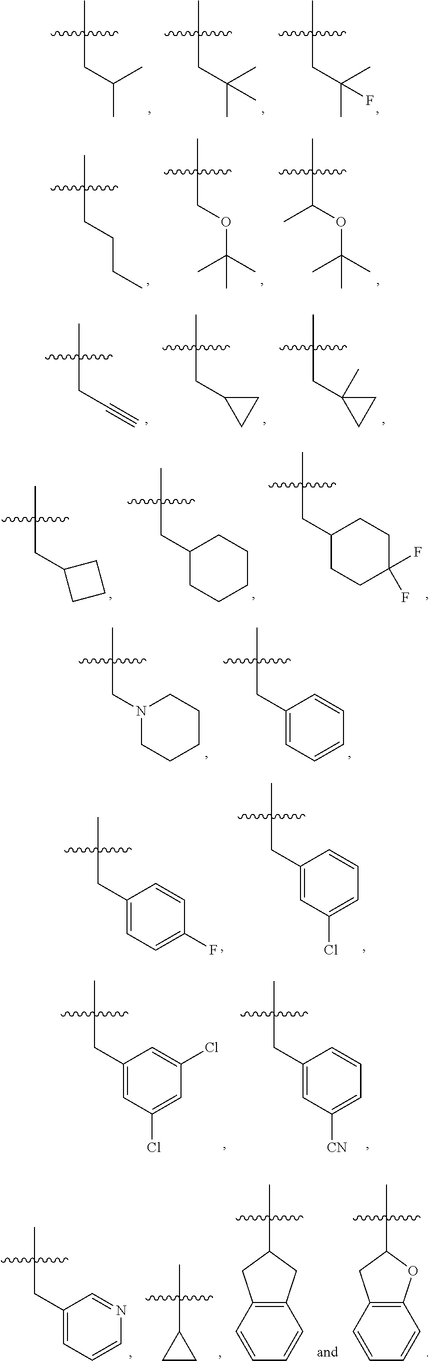

The carbon to which R² is attached can be a stereocenter. In some embodiments, the carbon to which R² is attached can be in the (S)-configuration. In other embodiments, the carbon to which R² is attached can be in the (R)-configuration. The stereo-version of a compound of Formula (I), or a pharmaceutically acceptable salt thereof, is provided below:

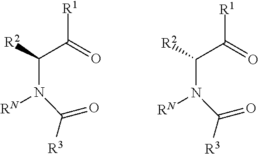

The substituent R⁴ can be various moieties. In some embodiments, R⁴ can be an unsubstituted ketoamide. In some embodiments, R⁴ can be a substituted ketoamide. The ketoamide can have the structure —C(═O)—C(═O)NR^{y1}R^{z1}. In some embodiments, R⁴ can be —C(═O)NH₂. In some embodiments, R⁴ can be an acyl, for example, R⁴ can be —C(═O)H, —C(═O)(an unsubstituted C₁₋₄ alkyl), —C(═O)(an unsubstituted to a substituted benzyl), —C(═O)(an unsubstituted to a substituted monocyclic heteroaryl) or —C(═O)(an unsubstituted to a substituted bicyclic heteroaryl). In some embodiments, R⁴ can be a substituted acyl. The acyl for R⁴ can have the structure —C(═O)R^{y2}. When the acyl is substituted, the possible groups that can be present on the acyl include hydroxy, a substituted or an unsubstituted alkoxy (such as —O-(an unsubstituted C₁₋₄ alkyl), —O-(an unsubstituted C₃₋₆ cycloalkyl), a substituted or an unsubstituted phenoxy or a substituted or an unsubstituted benzyloxy) or —O—(C═O)-(an unsubstituted C₁₋₆ alkyl).

R^{y1}, R^{y2} and R^{z1} can be a variety of groups. In some embodiments, R^{y1}, R^{y2} and R^{z1} can be independently selected from hydrogen, C₁₋₈ alkyl, C₂₋₈ alkenyl, C₂₋₈ alkynyl, C₃₋₈ cycloalkyl (for example, a monocyclic C₃₋₈ cycloalkyl), C₃₋₈ cycloalkenyl (such as a monocyclic C₃₋₈ cycloalkenyl), aryl (such as phenyl or naphthyl), heteroaryl (including a monocyclic or a bicyclic heteroaryl), heterocyclyl (for example, a monocyclic or a bicyclic heterocyclyl), aryl(alkyl) (such as benzyl), heteroaryl(alkyl) (including a monocyclic heteroaryl(CH₂)— and a monocyclic (heteroaryl(CH₂CH₂)—) or heterocyclyl(alkyl) (such as a monocyclic heterocyclyl(CH₂)— and a monocyclic heterocyclyl(CH₂CH₂)—), wherein each of the aforementioned R^{y1}, R^{y2} and R^{z1} groups can be unsubstituted or substituted. In some embodiments, R^{y1}, R^{y2} and R^{z1} can be independently selected from H, C₁₋₈ alkyl, an unsubstituted C₁₋₄ haloalkyl (including —CF₃, —CHF₂, —C(CH₃)F₂, —CH₂F, —CH(CH₃)F, —CH₂CF₃, —CH₂CH₂F and —CH₂CH₂CH₂F), —C₁₋₄ alkyl(OH) (including —CH₂OH, —CH₂CH₂OH and —CH(CH₃)OH), —C₁₋₄ alkyl(C₁₋₄ alkoxy) (such as —CH₂O(an unsubstituted C₁₋₄ alkyl) and —CH₂CH₂O(an unsubstituted C₁₋₄ alkyl)), —C₁₋₄ alkyl-O-(a monocyclic C₃₋₆ cycloalkyl) (such as —CH₂O(a monocyclic C₃₋₆ cycloalkyl), —CH₂CH₂O(a monocyclic C₃₋₆ cycloalkyl)), —C₁₋₄ alkyl-O-(phenyl) (for example, —CH₂O(phenyl) and —CH₂CH₂O(phenyl)), —C₁₋₄ alkyl-O-(5- to 6-membered monocyclic heteroaryl) (such as —CH₂O(5- to 6-membered monocyclic heteroaryl) and —CH₂CH₂O(5- to 6-membered monocyclic heteroaryl)), —C₁₋₄ alkyl-O-(5- to 6-membered monocyclic heterocyclyl) (for example, —CH₂O(5- to 6-membered monocyclic heterocyclyl) and —CH₂CH₂O(5- to 6-membered monocyclic heterocyclyl)), —C₁₋₄ alkyl-O-(a monocyclic C₃₋₆ cycloalkyl(C₁₋₄ alkyl) (such as —C₁₋₄ alkyl-O—CH₂-(monocyclic C₃₋₆ cycloalkyl) and —C₁₋₄ alkyl-O—CH₂CH₂-(monocyclic C₃₋₆ cycloalkyl)), —C₁₋₄ alkyl-O-(benzyl) (for example, —CH₂O(benzyl) and —CH₂CH₂O(benzyl)), —C₁₋₄ alkyl-O-(5- to 6-membered monocyclic heteroaryl(C₁₋₄ alkyl), —C₁₋₄ alkyl-O-(5- to 6-membered monocyclic heterocyclyl(C₁₋₄ alkyl), —C₁₋₄ alkyl-O(C═O)(an unsubstituted C₁₋₆ alkyl) (for example, —CH₂O(C═O)(an unsubstituted C₁₋₆ alkyl)), a monocyclic C₃₋₈ cycloalkyl (such as cyclopropyl, cyclobutyl, cyclopentyl, cyclohexyl, cycloheptyl and cyclooctyl), a monocyclic heteroaryl (such as imidazole, 1,3,4-oxadiazole and pyridinyl), a monocyclic heterocyclyl (for example, tetrahydrofuran and tetrahydropyran), a bicyclic heteroaryl (for example, benzothiazole, benzoimidazole and benzooxazole), a bicyclic heterocyclyl, a monocyclic C₃₋₆ cycloalkyl(alkyl), aryl(alkyl) (such as benzyl), heteroaryl(alkyl) (for example, a monocyclic heteroaryl-(CH₂)—, such as pyridinyl-(CH₂)—) and heterocyclyl(alkyl) (for example, a monocyclic heterocyclyl-(CH₂)—), wherein each of the aforementioned R^{y1}, R^{y2} and R^{z1} groups can be unsubstituted or substituted.

In some embodiments, R⁴ can be —C(═O)R^{y2}, wherein R^{y2} can be —C₁₋₄ alkyl(OH) (such as —CH₂OH). In some embodiments, R⁴ can be —C(═O)—C(═O)NR^{y1}R^{z1}; wherein R^{y1} can be H; and R^{z1} can be any of the moieties listed for R^{z1} in the previous paragraph. In some embodiments, R⁴ can be —C(═O)—C(═O)NR^{y1}R^{z1}; wherein R^{y1} can be H; and R^{z1} can be a monocyclic C₃₋₈ cycloalkyl (for example, cyclopropyl, cyclobutyl, cyclopentyl, cyclohexyl, cycloheptyl and cyclooctyl).

Prodrug-type and phosphate-containing moieties can be present at R⁴. In some embodiments, R⁴ can be —CH(OH)—(S(═O)₂—OH) or —CH(OH)—(S(═O)₂—O—). Those skilled in the art understand that when R⁴ is —CH(OH)—(S(═O)₂—O—), the negative charge can be balanced with a positive ion and form a salt. For example, R⁴ can be —CH(OH)—(S(═O)₂—O—)(Na⁺). In other embodiments, R⁴ can be —CH(OH)((P═O)(OR⁶)₂), wherein each R⁶ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl). In still other embodiments, R⁴ can be —C(═O)CH₂—O—((P═O)(OR⁷)₂), wherein each R⁷ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl). Other examples of R⁶ and R⁷ groups include methyl, ethyl, n-propyl, iso-propyl, n-butyl, iso-butyl, sec-butyl, tert-butyl, pentyl (straight-chained and branched), hexyl (straight-chained and branched), ethenyl, propenyl, butenyl, pentenyl, hexenyl, chloromethyl, fluoromethyl, difluoromethyl, dichloromethyl, trifluoromethyl, trichloromethyl, an unsubstituted or a substituted phenyl and an unsubstituted or a substituted benzyl.

In some embodiments, R⁴ can be cyano. In other embodiments, R⁴ can be an unsubstituted C₂₋₅ alkynyl. In still other embodiments, R⁴ can be a substituted C₂₋₅ alkynyl. The C₂₋₅ alkynyl can have various structures. For example, the C₂₋₅ alkynyl can have the structure —(CH₂)₁-C₂₋₄ alkynyl or —(CH₂)₂-C₂₋₃ alkynyl.

As provided herein, R³ can be

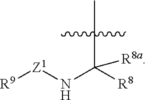

In some embodiments, R³ can be

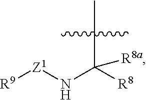

wherein Z¹ is —C(═O)— such that R³ can be

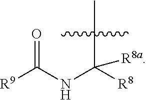

In some embodiments, R³ can be

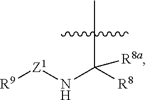

wherein Z¹ is —S(═O)₂— such that R³ can be

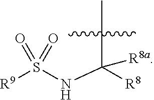

Depending upon R⁸, the carbon to which R⁸ is attached can be a chiral center. In some embodiments, R³ can be

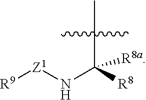

In other embodiments, R³ can be

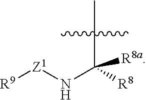

In some embodiments, R⁹ can be an unsubstituted C₁₋₆ haloalkyl. For example, R⁹ can be —CF₃, —CClF₂, —CHF₂, —C(CH₃)F₂, —CH₂F, —CH(CH₃)F, —CH₂CF₃, —CH(CH₃)CF₃, —CH₂CH₂CF₃, —CH₂CH(CH₃)CF₃, —CF₂CF₃, —CH₂CH₂F, —CF₂CF₂CF₃ and —CH₂CH₂CH₂F. In some embodiments, R⁹ can be —CF₃. In other embodiments, R⁹ can be a substituted C₁₋₆ haloalkyl where the C₁₋₆ haloalkyl can be substituted 1 or 2 times with an unsubstituted C₁₋₄ alkoxy. When the C₁₋₆ haloalkyl is substituted with 1 or 2 unsubstituted C₁₋₄ alkoxys, one or more hydrogens of the C₁₋₆ haloalkyl (for example, 1, 2 or 3 hydrogens) can be replaced with an unsubstituted C₁₋₄ alkoxy (for example —O(C₁₋₄ alkyl) such as methoxy, ethoxy, n-propoxy, isopropoxy, n-butoxy, isobutoxy, sec-butoxy and tert-butoxy). Exemplary C₁₋₆ haloalkyls substituted with an unsubstituted C₁₋₄ alkoxy include —C(OCH₃)F₂, —CH(OCH₃)F, —C(OCH₃)(CH₃)F, —CH(OCH₃)CF₃, —C(OCH₃)(CH₃)CF₃, —CH₂CH(OCH₃)CF₃, —CH₂C(OCH₃)(CH₃)CF₃, —CH₂CH(OCH₃)F and —CH₂CH₂CH(OCH₃)F. In still other embodiments, R⁹ can be an unsubstituted C₁₋₆ alkyl, such as methyl, ethyl, n-propyl, iso-propyl, n-butyl, iso-butyl, sec-butyl, tert-butyl, pentyl (straight-chained or branched) and hexyl (straight-chained or branched). In yet still other embodiments, R⁹ can be a C₁₋₆ alkyl substituted 1 or 2 times with an unsubstituted C₁₋₄ alkoxy and/or hydroxy. When the C₁₋₆ alkyl is substituted with an unsubstituted C₁₋₄ alkoxy and/or hydroxy, a hydrogen of the C₁₋₆ alkyl can be replaced with an unsubstituted C₁₋₄ alkoxy and/or hydroxy such as those described herein. A non-limiting list of C₁₋₆ alkyls substituted 1 or 2 times with an unsubstituted C₁₋₄ alkoxy include —CH(OH)(CH₃)₃, —CH₂(OCH₃), —CH(OCH₃)₂, —CH(CH₃)(OCH₃) and —C(CH₃)₂(OCH₃).

In some embodiments, R⁹ can be an unsubstituted phenyl. In some embodiments, R⁹ can be a substituted phenyl. When the phenyl is substituted, a variety of substituents can be present, and the number of substituents can vary. In some embodiments, R⁹ can be a phenyl substituted 1, 2, 3 or 4 times with a moiety independently selected from halogen, an unsubstituted C₁₋₆ alkyl, an unsubstituted C₁₋₆ haloalkyl, an unsubstituted C₁₋₆ alkoxy and an unsubstituted or a substituted monocyclic heteroaryl. For example, R⁹ can be a phenyl substituted 1, 2, 3 or 4 times with a moiety independently selected from F, Cl, Br, as methyl, ethyl, n-propyl, iso-propyl, n-butyl, iso-butyl, sec-butyl, tert-butyl, pentyl (straight-chained or branched), hexyl (straight-chained or branched), —CF₃, —CHF₂, —C(CH₃)F₂, —CH₂F, —CH(CH₃)F, —CH₂CF₃, —CH₂CH₂F, —CH₂CH₂CH₂F, —O(an unsubstituted C₁₋₆ alkyl) (such as methoxy, ethoxy, n-propoxy, iso-propoxy, n-butoxy, iso-butoxy, sec-butoxy, tert-butoxy, pentoxy (straight-chained or branched), hexoxy (straight-chained or branched)), and an unsubstituted or a substituted 5- to 6-membered monocyclic heteroaryl, wherein the heteroaryl can include 1, 2 or 3 heteroatoms selected from oxygen, sulfur and nitrogen. Exemplary monocyclic heteroaryls include furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine and pyrazine.

In some embodiments, R⁹ can be an unsubstituted or a substituted monocyclic heteroaryl. A variety of an unsubstituted or a substituted monocyclic heteroaryls can be present for R⁹. For example, the heteroaryl can be a 5- or 6-membered heteroaryl that includes 1, 2 or 3 heteroatoms selected from nitrogen (N), oxygen (O) and sulfur (S). Exemplary heteroaryls for an unsubstituted or a substituted monocyclic heteroaryl include, but are not limited to, furane, isoxazole, isothiazole, pyrrole, pyrazole, oxazole, thiazole, imidazole, 1,2,3-triazole, 1,2,4-triazole, imidazole, 1,3,4-oxadiazole, 1,3,4-thiadiazole, pyridine, pyridazine, pyrimidine, pyridazine and pyrazine. In yet still other embodiments, R⁹ can be an unsubstituted or a substituted monocyclic heterocyclyl. A non-limiting list of monocyclic heterocyclyls for R⁹ include azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine. Various substituents can be present on a substituted heteroaryl and/or a substituted heterocyclyl of R⁹. For example, the heteroaryl and/or heterocyclyl of R⁹ can be substituted 1, 2 or 3 times with a moiety selected from halogen, an unsubstituted C₁₋₆ alkyl, an unsubstituted C₁₋₆ haloalkyl, an unsubstituted C₁₋₆ alkoxy and an unsubstituted or a substituted phenyl. Suitable halogens, unsubstituted C₁₋₆ alkyls, unsubstituted C₁₋₆ haloalkyls and unsubstituted C₁₋₆ alkoxys are described herein, including those that can be present on a phenyl of R⁹. An unsubstituted or a substituted phenyl that can be substituted on a heteroaryl or a heterocyclyl of R⁹ can substituted 1, 2, 3, 4 or 5 times. A non-limiting list of examples of substituents that can be substituted on a phenyl that is substituted on a heteroaryl or heterocyclyl of R⁹ include F, Cl, Br, as methyl, ethyl, n-propyl, iso-propyl, n-butyl, iso-butyl, sec-butyl, tert-butyl, pentyl (straight-chained or branched), hexyl (straight-chained or branched), —CF₃, —CHF₂, —C(CH₃)F₂, —CH₂F, —CH(CH₃)F, —CH₂CF₃, —CH₂CH₂F, —CH₂CH₂CH₂F, —O(an unsubstituted C₁₋₆ alkyl) (such as methoxy, ethoxy, n-propoxy, iso-propoxy, n-butoxy, iso-butoxy, sec-butoxy, tert-butoxy, pentoxy (straight-chained or branched), hexoxy (straight-chained or branched)), and an unsubstituted or a substituted 5- to 6-membered monocyclic heteroaryl, wherein the heteroaryl can include 1, 2 or 3 heteroatoms selected from oxygen, sulfur and nitrogen. Examples of monocyclic heteroaryls include furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine and pyrazine.

In some embodiments, R⁹ can be an unsubstituted monocyclic C₃₋₆ cycloalkyl, such as cyclopropyl, cyclobutyl, cyclopentyl and cyclohexyl. In other embodiments, R⁹ can be a halogen-substituted monocyclic C₃₋₆ cycloalkyl. In still other embodiments, R⁹ can be a monocyclic C₃₋₆ cycloalkyl substituted with an unsubstituted C₁₋₄ alkyl. In yet still other embodiments, R⁹ can be a monocyclic C₃₋₆ cycloalkyl substituted with an unsubstituted C₁₋₄ alkoxy. In some embodiments, R⁹ can be a monocyclic C₃₋₆ cycloalkyl substituted with an unsubstituted C₂₋₄ alkenyl. In other embodiments, R⁹ can be a monocyclic C₃₋₆ cycloalkyl substituted with an unsubstituted C₁₋₄ haloalkyl. In still other embodiments, R⁹ can be a monocyclic C₃₋₆ cycloalkyl substituted with an unsubstituted monocyclic C₃₋₆ cycloalkyl, such as cyclopropyl, cyclobutyl, cyclopentyl or cyclohexyl. In some embodiments, R⁹ can be an unsubstituted bicyclic C₅₋₆ cycloalkyl. In other embodiments, R⁹ can be a substituted bicyclic C₅₋₆ cycloalkyl. The two rings of a bicyclic C₅₋₆ cycloalkyl can be connected in a spiro-fashion or a fused-fashion. In some embodiments, R⁹ can be a halogen-substituted bicyclic C₅₋₆ cycloalkyl. In still other embodiments, R⁹ can be a bicyclic C₅₋₆ cycloalkyl substituted with an unsubstituted C₁₋₄ alkyl. In yet still other embodiments, R⁹ can be a bicyclic C₅₋₆ cycloalkyl substituted with an unsubstituted C₁₋₄ alkoxy. In some embodiments, R⁹ can be a bicyclic C₅₋₆ cycloalkyl substituted with an unsubstituted C₂₋₄ alkenyl. In other embodiments, R⁹ can be a bicyclic C₅₋₆ cycloalkyl substituted with an unsubstituted C₁₋₄ haloalkyl. In still other embodiments, R⁹ can be a bicyclic C₅₋₆ cycloalkyl substituted with an unsubstituted monocyclic C₃₋₆ cycloalkyl (including cyclopropyl, cyclobutyl, cyclopentyl and cyclohexyl). A non-liming list of bicyclic C₅₋₆ cycloalkyls include spiro[2.2]pentane, spiro[2.3]hexane, bicyclo[1.1.1]pentane and bicyclo[2.1.1]hexane.

Suitable halogen-substituted monocyclic C₃₋₆ cycloalkyls include halogen-substituted cyclopropyl, halogen-substituted cyclobutyl, halogen-substituted cyclopentyl and halogen-substituted cyclohexyl. Additional monocyclic C₃₋₆ cycloalkyls include cyclopropyl substituted with an unsubstituted C₁₋₄ alkyl, an unsubstituted C₂₋₄ alkenyl, an unsubstituted C₁₋₄ alkoxy, an unsubstituted C₁₋₄ haloalkyl and/or an unsubstituted monocyclic C₃₋₆ cycloalkyl, cyclobutyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl, cyclopentyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl and cyclohexyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl. The number halogens on a halogen-substituted monocyclic C₃₋₆ cycloalkyl and/or a bicyclic C₅₋₆ cycloalkyl, the number of unsubstituted C₁₋₄ alkyls on a monocyclic C₃₋₆ cycloalkyl and/or a bicyclic C₅₋₆ cycloalkyl, the number of unsubstituted C₁₋₄ alkoxys on a monocyclic C₃₋₆ cycloalkyl and/or a bicyclic C₅₋₆ cycloalkyl, the number of unsubstituted C₂₋₄ alkenyls on a monocyclic C₃₋₆ cycloalkyl and/or a bicyclic C₅₋₆ cycloalkyl, the number of unsubstituted C₁₋₄ haloalkyls on a monocyclic C₃₋₆ cycloalkyl and/or a bicyclic C₅₋₆ cycloalkyl and the number of unsubstituted monocyclic C₃₋₆ cycloalkyls on a monocyclic C₃₋₆ cycloalkyl and/or a bicyclic C₅₋₆ cycloalkyl can vary. For example, 1, 2, 3 or 4 halogens can be present on a halogen-substituted monocyclic C₃₋₆ cycloalkyl, 1, 2, 3 or 4 unsubstituted C₁₋₄ alkyls can be present on a monocyclic C₃₋₆ cycloalkyl substituted with an unsubstituted C₁₋₄ alkyl, 1, 2, 3 or 4 unsubstituted C₁₋₄ alkoxys can be present on a monocyclic C₃₋₆ cycloalkyl substituted with an unsubstituted C₁₋₄ alkoxy, 1, 2, 3 or 4 unsubstituted C₂-4 alkenyls can be present on a monocyclic C₃₋₆ cycloalkyl substituted with an unsubstituted C₂₋₄ alkenyl, 1, 2, 3 or 4 unsubstituted C₁₋₄ haloalkyls can be present on a monocyclic C₃₋₆ cycloalkyl substituted with an unsubstituted C₁₋₄ haloalkyl, 1 or 2 unsubstituted monocyclic C₃₋₆ cycloalkyls can be present on a monocyclic C₃₋₆ cycloalkyl, 1, 2, 3 or 4 halogens can be present on a halogen-substituted bicyclic C₅₋₆ cycloalkyl, 1, 2, 3 or 4 unsubstituted C₁₋₄ alkyls can be present on a bicyclic C₅₋₆ cycloalkyl substituted with an unsubstituted C₁₋₄ alkyl, 1, 2, 3 or 4 unsubstituted C₁₋₄ alkoxys can be present on a bicyclic C₅₋₆ cycloalkyl substituted with an unsubstituted C₁₋₄ alkoxy, 1, 2, 3 or 4 unsubstituted C₂₋₄ alkenyls can be present on a bicyclic C₅₋₆ cycloalkyl substituted with an unsubstituted C₂₋₄ alkenyl, 1, 2, 3 or 4 unsubstituted C₁₋₄ haloalkyls can be present on a bicyclic C₅₋₆ cycloalkyl substituted with an unsubstituted C₁₋₄ haloalkyl and 1 or 2 unsubstituted monocyclic C₃₋₆ cycloalkyls can be present on a bicyclic C₅_₆ cycloalkyl. In some embodiments, a monocyclic C₃₋₆ cycloalkyl can be substituted with 1 or more substituents (such as 1, 2, 3 or 4 substituents) selected from halogen, an unsubstituted C₁₋₄ alkyl, an unsubstituted C₁₋₄ alkoxy, an unsubstituted C₂₋₄ alkenyl, and an unsubstituted C₁₋₄ haloalkyl. In other embodiments, a bicyclic C₅₋₆ cycloalkyl can be substituted with 1 or more substituents (such as 1, 2, 3 or 4 substituents) selected from halogen, an unsubstituted C₁₋₄ alkyl, an unsubstituted C₁₋₄ alkoxy an unsubstituted C₂₋₄ alkenyl, and an unsubstituted C₁₋₄ haloalkyl. Suitable halogens that can be present on a substituted monocyclic C₃₋₆ cycloalkyl include, but are not limited to, fluoro (F) and chloro (C₁). Examples of unsubstituted C₁₋₄ haloalkyls include, but are not limited to, —CF₃, —CHF₂, —C(CH₃)F₂, —CH₂F, —CH(CH₃)F, —CH₂CF₃, —CH₂CH₂F and —CH₂CH₂CH₂F.

In some embodiments, R⁹ can be an unsubstituted alkoxy. In other embodiments, R⁹ can be a substituted alkoxy. Various alkoxys can be present for R⁹. For example, —O-(hydrocarbon) (such as —O—(C₁₋₈ alkyl)), —O-(monocyclic C₃₋₈ cycloalkyl), —O-(bicyclic C₅₋₈ cycloalkyl), —O-(phenyl), —O-(bicyclic aryl), —O-(monocyclic heteroaryl), —O-(bicyclic heteroaryl), —O-(monocyclic heterocyclyl) and —O-(bicyclic heterocyclyl). A non-limiting list of examples of C₁₋₆ alkoxys are methoxy, ethoxy, n-propoxy, iso-propoxy, n-butoxy, iso-butoxy, sec-butoxy, tert-butoxy, pentoxy (straight-chained or branched), hexoxy (straight-chained or branched), —O-cyclopropyl, —O-cyclobutyl, —O-cyclopentyl, —O— cyclohexyl and —O-(bicyclo[1.1.1]pentyl). In some embodiments, R⁹ can be —O-(an unsubstituted or a substituted C₁₋₈ alkyl). In some embodiments, R⁹ can be —O-(an unsubstituted C₁₋₄ alkyl). A variety of substituents can be present on a substituted alkoxy for R⁹. Examples of suitable substituents are those provided for “optionally substituted.” In some embodiments, 1, 2, 3 or 4 substituents can be present on a substituted alkoxy. For example, a substituted alkoxy can be substituted 1, 2, 3 or 4 times with substituents independently selected from halogen (for example, F or Cl), hydroxy, an unsubstituted C₁₋₄ alkyl and an unsubstituted C₁₋₄ haloalkyl. In some embodiments, R⁹ can be an unsubstituted or substituted alkoxy selected from methoxy, ethoxy, n-propoxy, iso-propoxy, n-butoxy, iso-butoxy, sec-butoxy, tert-butoxy, —O-cyclopropyl, —O-cyclobutyl, —O-cyclopentyl, —O-cyclohexyl and —O-(bicyclo[1.1.1]pentyl), —O-(phenyl) and —O-(halo-substituted phenyl).

In some embodiments, R⁹ can be an amino or an amine, such as —NR¹⁷R¹⁸, wherein R¹⁷ and R¹⁸ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl). In other embodiments, R⁹ can be —NR¹⁷R¹⁸, wherein R¹⁷ and R¹⁸ are taken together along with the nitrogen to which they are connected to form an unsubstituted or a substituted 3-8 membered heterocyclyl.

In some embodiments, R¹⁷ and/or R¹⁸ can be an unsubstituted C₁₋₈ alkyl. In other embodiments, R¹⁷ and/or R¹⁸ can be a substituted C₁₋₈ alkyl. In still other embodiments, R¹⁷ and/or R¹⁸ can be an unsubstituted C₂₋₈ alkenyl. In yet still other embodiments, R¹⁷ and/or R¹⁸ can be a substituted C₂₋₈ alkenyl. In some embodiments, R¹⁷ and/or R¹⁸ can be an unsubstituted C₂₋₈ alkynyl. In other embodiments, R¹⁷ and/or R¹⁸ can be a substituted C₂₋₈ alkynyl. In still other embodiments, R¹⁷ and/or R¹⁸ can be an unsubstituted C₃₋₈ cycloalkyl, for example an unsubstituted monocyclic C₃₋₈ cycloalkyl. In yet still other embodiments, R¹⁷ and/or R¹⁸ can be a substituted C₃₋₈ cycloalkyl, for example a substituted monocyclic C₃₋₈ cycloalkyl. Various cyclic moieties can be present for R¹⁷ and/or R¹⁸. In some embodiments, R¹⁷ and/or R¹⁸ can be an unsubstituted aryl. In other embodiments, R¹⁷ and/or R¹⁸ can be a substituted aryl. In still other embodiments, R¹⁷ and/or R¹⁸ can be an unsubstituted heteroaryl. In yet still other embodiments, R¹⁷ and/or R¹⁸ can be a substituted heteroaryl. In some embodiments, R¹⁷ and/or R¹⁸ can be an unsubstituted 3- to 8-membered monocyclic heterocyclyl. In other embodiments, R¹⁷ and/or R¹⁸ can be a substituted 3- to 8-membered monocyclic heterocyclyl. In still other embodiments, R¹⁷ and/or R¹⁸ can be an unsubstituted aryl(alkyl). In yet still other embodiments, R¹⁷ and/or R¹⁸ can be a substituted aryl(alkyl). In some embodiments, R¹⁷ and/or R¹⁸ can be an unsubstituted heteroaryl(alkyl). In other embodiments, R¹⁷ and/or R¹⁸ can be a substituted heteroaryl(alkyl). The aryl, heteroaryl and heterocyclyl can be monocyclic or bicyclic, and include 1, 2, 3, 4 or 5 heteroatoms independently selected from O (oxygen), S (sulfur) and N (nitrogen). When R¹⁷ and/or R¹⁸ is aryl(alkyl) or heteroaryl(alkyl), the alkyl linker can be 1, 2 or 3 alkylene groups, such as —CH₂-, —CH₂CH₂— and —CH₂CH₂CH₂—. In some embodiments, R⁹ can be —NHR¹⁸, wherein R¹⁸ can be as provided herein. For example, In some embodiments, R⁹ can be —NHR¹⁸, wherein R¹⁸ can be an unsubstituted C₁₋₈ alkyl.

In some embodiments, R³ can be

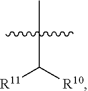

wherein R¹⁰ can be independently selected from an unsubstituted or a substituted C₁₋₆ alkyl, an unsubstituted or a substituted C₂₋₆ alkenyl, an unsubstituted or a substituted C₂₋₆ alkynyl, an unsubstituted or a substituted monocyclic C₃₋₆ cycloalkyl, an unsubstituted or a substituted bicyclic C₅₋₈ cycloalkyl and an unsubstituted or a substituted monocyclic 4- to 6-membered heterocyclyl, wherein when the C₁₋₆ alkyl is substituted, the C₁₋₆ alkyl can be substituted 1, 2, 3 or 4 times with a substituent independently selected from halogen, cyano, —NH₂, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic 4-6 membered heterocyclyl, an unsubstituted or a substituted monocyclic 5- or 6-membered heteroaryl, an unsubstituted C₁₋₄ alkoxy, an unsubstituted or a substituted phenoxy, an unsubstituted or a substituted —O—(CH₂)-phenyl and an unsubstituted C₁₋₄ haloalkoxy; wherein when the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted, the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl can be substituted 1, 2, 3 or 4 times with a substituent independently selected from halogen, an unsubstituted C₁₋₄ alkyl, an unsubstituted C₂₋₄ alkenyl, an unsubstituted C₂₋₄ alkynyl, an unsubstituted C₁₋₄ haloalkyl and an unsubstituted C₁₋₄ alkoxy; and R¹¹ can be —(NH)ₘ-(an unsubstituted or a substituted 5- to 10-membered heteroaryl), wherein m can be 1. In some embodiments, R¹¹ can be an unsubstituted or a substituted monocyclic 4- to 6-membered heterocyclyl. Examples of heterocyclyls for R¹¹ include unsubstituted or a substituted 4- to 6-membered monocyclic heterocyclyls that include 1, 2 or 3 heteroatoms independently selected from N (nitrogen), O (oxygen) and S (sulfur). A non-limiting list of heterocyclyl for R¹¹ include the following: azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine. In some embodiment, m can be 1; and R¹¹ can be an —(NH)-(unsubstituted 5- to 6-membered monocyclic heteroaryl). In other embodiments, m can be 1; and R¹¹ can be a —(NH)-(substituted 5- to 6-membered monocyclic heteroaryl). In still other embodiment, m can be 1; and R¹¹ can be an —(NH)-(unsubstituted 8- to 10-membered bicyclic heteroaryl). In other embodiments, m can be 1; and R¹¹ can be a —(NH)-(substituted 8- to 10-membered bicyclic heteroaryl). An example of a 5- to 10-membered heteroaryl that can be present for R¹¹ include a 5- to 10-membered heteroaryl that includes 1, 2 or 3 heteroatoms independently selected from N (nitrogen), O (oxygen) and S (sulfur). Examples of suitable 5- to 6-membered monocyclic heteroaryls include, but are not limited to, furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine, pyrazine, indole, quinoline, isoquinoline and quinazoline. In still other embodiments, R¹¹ can be —O-(an unsubstituted or a substituted C₁₋₆ alkyl). In yet still other embodiments, R¹¹ can be —O-(an unsubstituted or a substituted C₃₋₈ cycloalkyl). In some embodiments, R¹¹ can be —O—(C₁₋₄ alkyl)-(an unsubstituted or a substituted C₃₋₈ cycloalkyl). The cycloalkyl of —O-(an unsubstituted or a substituted C₃₋₈ cycloalkyl) and —O—(C₁₋₄ alkyl)-(an unsubstituted or a substituted C₃₋₈ cycloalkyl)can be a monocyclic C₃₋₆ cycloalkyl or a bicyclic C₅₋₈ cycloalkyl. The C₁₋₄ alkyl of —O—(C₁₋₄ alkyl)-(an unsubstituted or a substituted C₃₋₈ cycloalkyl) can be —CH₂—, —CH₂CH₂—, —CH₂CH₂CH₂— or —CH₂CH₂CH₂CH₂—.

As described herein, R¹¹ can be substituted. Exemplary groups that can be present on R¹¹ include halogen, an unsubstituted C₁₋₄ alkyl (for example, methyl, ethyl, n-propyl, iso-propyl, n-butyl, iso-butyl, sec-butyl and tert-butyl), an unsubstituted monocyclic C₃₋₆ cycloalkyl (such as cyclopropyl, cyclobutyl, cyclopentyl and cyclohexyl), an unsubstituted C₁₋₄ alkoxy (for example —O(C₁₋₄ alkyl), such as methoxy, ethoxy, n-propoxy, iso-propoxy, n-butoxy, iso-butoxy, sec-butoxy and tert-butoxy), an unsubstituted C₁₋₄ haloalkyl (for example, —CF₃, —CHF₂, —C(CH₃)F₂, —CH₂F, —CH(CH₃)F, —CH₂CF₃, —CH₂CH₂F and —CH₂CH₂CH₂F), an unsubstituted phenyl, a substituted phenyl, an unsubstituted 5- or 6-membered heteroaryl and a substituted 5- or 6-membered heteroaryl (for example wherein a substituted phenyl and/or substituted 5- or 6-membered heteroaryl can be substituted 1, 2, 3, 4 or 5 times with a substituent independently selected from halogen (for example, F, Cl and Br), an unsubstituted C₁₋₄ alkyl (for example, methyl, ethyl, n-propyl, iso-propyl, n-butyl, iso-butyl, sec-butyl and tert-butyl), an unsubstituted C₁₋₄ alkoxy (for example —O(C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted —O(an unsubstituted C₁₋₄ haloalkyl) (for example, —O(C₁₋₄ haloalkyl) such as —OCF₃, —OCHF₂, —OC(CH₃)F₂, —OCH₂F, —OCH(CH₃)F, —OCH₂CF₃, —OCH₂CH₂F and —OCH₂CH₂CH₂F) and —S(═O)₂(an unsubstituted C₁₋₄ alkyl).

The R⁸ and R¹⁰ moieties can be a substituted or an unsubstituted version of a C_{1-6 alkyl}, a C₂₋₆ alkenyl, a C₂₋₆ alkynyl, a monocyclic C₃₋₆ cycloalkyl, a bicyclic C₅₋₈ cycloalkyl or a monocyclic 4- to 6-membered heterocyclyl. In some embodiments, R⁸ and/or R¹⁰ can be an unsubstituted C₁₋₆ alkyl. In other embodiments, R⁸ and/or R¹⁰ can be a substituted C₁₋₆ alkyl. Exemplary C₁₋₆ alkyls include methyl, ethyl, n-propyl, isopropyl, n-butyl, isobutyl, sec-butyl, tert-butyl, pentyl (straight-chained and branched) and hexyl (straight-chained and branched). In some embodiments, R⁸ and/or R¹⁰ can be an unsubstituted C₂₋₆ alkenyl. In other embodiments, R⁸ and/or R¹⁰ can be a substituted C₂₋₆ alkenyl. In still other embodiments, R⁸ and/or R¹⁰ can be an unsubstituted C₂₋₆ alkynyl. In yet still other embodiments, R⁸ and/or R¹⁰ can be a substituted C₂₋₆ alkynyl.

Cyclic moieties, including monocyclic and bicyclic moieties, can also be present for R⁸ and/or R¹⁰. In some embodiments, R⁸ and/or R¹⁰ can be an unsubstituted monocyclic C₃₋₆ cycloalkyl. In other embodiments, R⁸ and/or R¹⁰ can be a substituted monocyclic C₃₋₆ cycloalkyl. For example, R⁸ and/or R¹⁰ can be a substituted or an unsubstituted cyclopropyl, a substituted or an unsubstituted cyclobutyl, a substituted or an unsubstituted cyclopentyl or a substituted or an unsubstituted cyclohexyl. In some embodiments, R⁸ and/or R¹⁰ can be an unsubstituted bicyclic C₅₋₈ cycloalkyl. In other embodiments, R⁸ and/or R¹⁰ can be an unsubstituted bicyclic C₅₋₈ cycloalkyl. The two rings of the bicyclic C₅₋₈ cycloalkyl can joined in a fused or a spiro-fashion. Examples of rings connected in a fused and a spiro-fashion are provided herein. In some embodiments, R⁸ and/or R¹⁰ can be an unsubstituted or a substituted bicyclo[1.1.1]pentyl. In still other embodiments, R⁸ and/or R¹⁰ can be an unsubstituted monocyclic 4- to 6-membered heterocyclyl. In yet still other embodiments, R⁸ and/or R¹⁰ can be an unsubstituted monocyclic 4- to 6-membered heterocyclyl. The number of heteroatoms present in a monocyclic 4- to 6-membered heterocyclyl for R⁸ and/or R¹⁰ can vary. Suitable heteroatoms include, but are not limited to, O (oxygen), S (sulfur) and N (nitrogen). Examples of monocyclic 4- to 6-membered heterocyclyls are azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine (including unsubstituted or substituted versions of each of the aforementioned).

As described herein, R⁸ and/or R¹⁰ can be substituted. In some embodiments, when R⁸ and/or R¹⁰ is a C₁₋₆ alkyl that is substituted, the C₁₋₆ alkyl can be substituted 1, 2, 3 or 4 times with a substituent independently selected from halogen, cyano, —NH₂, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic 5- to 6-membered heteroaryl, an unsubstituted or a substituted monocyclic 4-6 membered heterocyclyl, an unsubstituted C₁₋₄ alkoxy, an unsubstituted or a substituted phenoxy, an unsubstituted or a substituted —O—(CH₂)-phenyl and an unsubstituted C₁₋₄ haloalkoxy. In some embodiments, R⁸ and/or R¹⁰ can be a C₁₋₆ alkyl that is substituted 1 to 13 times with deuterium. In some embodiments, R⁸ and/or R¹⁰ can be a C₁₋₆ alkyl that is substituted 1 to 9 times with deuterium, 1 to 6 times with deuterium, 1 to 5 times with deuterium or 1 to 3 times with deuterium. Each halogen that can be substituted on R⁸ and/or R¹⁰ moiety can be independently F (fluoro) or C₁ (chloro). In other embodiment, R⁸ and/or R¹⁰ can be a C₁₋₆ alkyl that is substituted with —NH₂. For example, R⁸ and/or R¹⁰ can be —(CH₂)₁₋₆-NH₂. Exemplary unsubstituted and substituted monocyclic C₃₋₆ cycloalkyls and unsubstituted and substituted bicyclic C₅₋₆ cycloalkyls that can be present on a substituted C₁₋₆ alkyl for R⁸ and/or R¹⁰ include cyclopropyl, cyclobutyl, cyclopentyl, cyclohexyl, halogen-substituted monocyclic C₃₋₆ cycloalkyls, (an unsubstituted C₁₋₄ alkyl)-substituted monocyclic C₃₋₆ cycloalkyls, bicyclo[1.1.1]pentyl and spiro[2.2]pentyl. A phenyl that can be substituted on a C₁₋₆ alkyl of R⁸ and/or R¹⁰. The phenyl can be unsubstituted or substituted. In some embodiments, R⁸ and/or R¹⁰ can be a C₁₋₆ alkyl that is substituted with an unsubstituted or a substituted monocyclic 5- to 6-membered heteroaryl. The monocyclic 5- to 6-membered heteroaryl can include various heteroatoms (such as nitrogen, oxygen and sulfur), and the number of heteroatoms can also vary. Exemplary monocyclic heteroaryls include furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine and pyrazine. In other embodiments, R⁸ and/or R¹⁰ can be a C₁₋₆ alkyl that is substituted with an unsubstituted or a substituted monocyclic 4-6 membered heterocyclyl. Examples of monocyclic 4- to 6-membered heterocyclyls are azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine (including unsubstituted or substituted versions of each of the aforementioned).

The phenyl, monocyclic 5- to 6-membered heteroaryl and/or monocyclic 4-6 membered heterocyclyl that can be substituted on a C₁₋₆ alkyl of R⁸ and/or R¹⁰ can be substituted one or more times (1, 2, 3 or 4 times) with a substituent independently selected from halogen (such as bromo, chloro and fluoro), cyano, an unsubstituted C₁₋₄ alkyl (for example, methyl, ethyl, n-propyl, iso-propyl, n-butyl, iso-butyl, sec-butyl and tert-butyl), hydroxy, an unsubstituted C₁₋₄ alkoxy (for example, —O(C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F) and an unsubstituted C₁₋₄ haloalkoxy (for example, —O(C₁₋₄ haloalkyl), such as —OCF₃, —OCHF₂, —OC(CH₃)F₂, —OCH₂F, —OCH(CH₃)F, —OCH₂CF₃, —OCH₂CH₂F and —OCH₂CH₂CH₂F).

Suitable unsubstituted C₁₋₄ alkoxys that can be substituted on a C₁₋₆ alkyl of R⁸ and/or R¹⁰ include methoxy, ethoxy, n-propoxy, isopropoxy, n-butoxy, isobutoxy, sec-butoxy and tert-butoxy. In some embodiments, a C₁₋₆ alkyl of R⁸ and/or R¹⁰ can be substituted with an unsubstituted or a substituted phenoxy. In other embodiments, a C₁₋₆ alkyl of R⁸ and/or R¹⁰ can be substituted with an unsubstituted or a substituted —O—(CH₂)-phenyl. In other embodiments, a C₁₋₆ alkyl of R⁸ and/or R¹⁰ can be substituted with an unsubstituted C₁₋₄ haloalkoxy. Examples of an unsubstituted C₁₋₄ haloalkoxy can be substituted on a C₁₋₆ alkyl of R⁸ and/or R¹⁰ include —OCF₃, —OCH₂F and —OCHF₂. In some embodiments, R⁸ and/or R¹⁰ can be an unsubstituted monocyclic C₃₋₆ cycloalkyl(CH₂)—. Various monocyclic C₃₋₆ cycloalkyl are described herein. As examples, R⁸ and/or R¹⁰ can be selected from cyclopropyl(CH₂)—, cyclobutyl(CH₂)—, cyclopentyl(CH₂)— and cyclohexyl(CH₂)—. In other embodiments, R⁸ and/or R¹⁰ can be (an unsubstituted or a substituted bicyclic C₅₋₆ cycloalkyl)-(CH₂)—. In still other embodiments, R⁸ and/or R¹⁰ can be (an unsubstituted or a substituted phenyl)-(CH₂)—. In still other embodiments, R⁸ and/or R¹⁰ can be (an unsubstituted or a substituted monocyclic 5- or 6-membered heteroaryl)-(CH₂)—. In yet still other embodiments, R⁸ and/or R¹⁰ can be (an unsubstituted or a substituted monocyclic 4-6 membered heterocyclyl)-(CH₂)—.

In some embodiments, when R⁸ and/or R¹⁰ is a substituted C₂₋₆ alkenyl, a substituted C₂₋₆ alkynyl, a substituted monocyclic C₃₋₆ cycloalkyl, a substituted bicyclic C₅₋₈ cycloalkyl or a substituted monocyclic 4- to 6-membered heterocyclyl, each of the aforementioned can be substituted 1, 2, 3 or 4 times with a substituents independently selected from halogen, an unsubstituted C₁₋₄ alkyl, an unsubstituted C₂₋₄ alkenyl, an unsubstituted C₂₋₄ alkynyl, an unsubstituted C₁₋₄ haloalkyl, an unsubstituted or a substituted monocyclic C₃₋₆ cycloalkyl and an unsubstituted C₁₋₄ alkoxy. Examples of unsubstituted C₁₋₄ alkyls, an unsubstituted C₂₋₄ alkenyl and an unsubstituted C₂₋₄ alkynyl that can be substituted on a substituted C₂₋₆ alkenyl, a substituted C₂₋₆ alkynyl, a substituted monocyclic C₃₋₆ cycloalkyl, a substituted bicyclic C₅₋₈ cycloalkyl or a substituted monocyclic 4- to 6-membered heterocyclyl include methyl, ethyl, n-propyl, isopropyl, n-butyl, iso-butyl, sec-butyl, tert-butyl, ethenyl, propenyl, butenyl, ethynyl, propynyl and butynyl. Suitable halogens and unsubstituted C₁₋₄ alkoxys that can be present on a substituted C₂₋₆ alkenyl, a substituted C₂₋₆ alkynyl, a substituted monocyclic C₃₋₆ cycloalkyl, a substituted bicyclic C₅₋₈ cycloalkyl or a substituted monocyclic 4- to 6-membered heterocyclyl are described herein, such as in the previous paragraph. Non-limiting list of unsubstituted and substituted monocyclic C₃₋₆ cycloalkyls include cyclopropyl, cyclobutyl, cyclopentyl, cyclohexyl and halogen-substituted monocyclic C₃₋₆ cycloalkyls. Examples of unsubstituted C₁₋₆ haloalkyls that can be present on a substituted C₂₋₆ alkenyl, a substituted C₂₋₆ alkynyl, a substituted monocyclic C₃₋₆ cycloalkyl, a substituted bicyclic C₅₋₈ cycloalkyl or a substituted monocyclic 4- to 6-membered heterocyclyl include, but are not limited to, —CF₃, —CHF₂, —C(CH₃)F₂, —CH₂F, —CH(CH₃)F, —CH₂CF₃, —CH₂CH₂F and —CH₂CH₂CH₂F.

In some embodiments, R^{8a} can be hydrogen. In other embodiments, R^{8a} can be an unsubstituted C₁₋₄ alkyl. For example, R^{8a} can be methyl, ethyl, n-propyl, isopropyl, n-butyl, iso-butyl or sec-butyl. In some embodiments, R⁸ and R^{8a} can be taken together to form an unsubstituted monocyclic C₃₋₆ cycloalkyl. In some embodiments, R⁸ and R^{8a} can be taken together to form a halogen-substituted monocyclic C₃₋₆ cycloalkyl, where 1, 2, 3 or 4 halogens can be present. As an example, R⁸ and R^{8a} can be taken together to form a fluoro-substituted monocyclic C₃₋₆ cycloalkyl.

In some embodiments, R³ can be R¹². As described herein, R¹² can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃_8 cycloalkyl, an unsubstituted or a substituted aryl, an unsubstituted or a substituted heteroaryl, an unsubstituted or a substituted 3- to 8-membered monocyclic heterocyclyl, an unsubstituted or a substituted 5- to 8-membered bicyclic heterocyclyl, an unsubstituted or a substituted aryl(alkyl), an unsubstituted or a substituted heteroaryl(alkyl), an unsubstituted or a substituted C-carboxy, —OR¹³, —NR¹⁴R¹⁵ or —C(═O)—NR^{16A}R^{16B}. In some embodiments, R¹² can be an unsubstituted C₁₋₈ alkyl. In other embodiments, R¹² can be a substituted C₁₋₈ alkyl. In still other embodiments, R¹² can be an unsubstituted C₂₋₈ alkenyl. In yet still other embodiments, R¹² can be a substituted C₂₋₈ alkenyl. In some embodiments, R¹² can be an unsubstituted C₂₋₈ alkynyl. In other embodiments, R¹² can be a substituted C₂₋₈ alkynyl.

A variety of cyclic moieties can be present for R¹². In some embodiments, R¹² can be an unsubstituted C₃₋₈ cycloalkyl. In other embodiments, R¹² can be a substituted C₃₋₈ cycloalkyl. For example, R¹² can be an unsubstituted or a substituted monocyclic C₃₋₈ cycloalkyl. In still other embodiments, R¹² can be an unsubstituted aryl. In yet still other embodiments, R¹² can be a substituted aryl. As an example, R¹² can be an unsubstituted or a substituted phenyl. In some embodiments, R¹² can be an unsubstituted heteroaryl. In other embodiments, R¹² can be a substituted heteroaryl. In some embodiments, R¹² can be an unsubstituted 3- to 12-membered monocyclic heterocyclyl. In other embodiments, R¹² can be a substituted 3- to 12-membered monocyclic heterocyclyl. In still other embodiments, R¹² can be an unsubstituted or a substituted 5- to 12-membered bicyclic heterocyclyl. In yet still other embodiments, R¹² can be an unsubstituted or a substituted 5- to 12-membered bicyclic heterocyclyl. In some embodiments, R¹² can be an unsubstituted or a substituted 3- to 12-membered monocyclic heterocyclyl. In other embodiments, R¹² can be an unsubstituted or a substituted 5- to 8-membered bicyclic heterocyclyl. In some embodiments, R¹² can be an unsubstituted aryl(alkyl). In other embodiments, R¹² can be a substituted aryl(alkyl). For example, R¹² can be an unsubstituted or a substituted benzyl. In some embodiments, R¹² can be an unsubstituted heteroaryl(alkyl). In other embodiments, R¹² can be a substituted heteroaryl(alkyl). In some embodiments, R¹² can be an unsubstituted heterocyclyl(alkyl). In other embodiments, R¹² can be a substituted heterocyclyl(alkyl). The aryl, heteroaryl and heterocyclyl, including that those of an aryl(alkyl), heteroaryl(alkyl) and heterocyclyl(alkyl)) can be monocyclic or bicyclic (unless stated otherwise), and include 1, 2, 3, 4 or 5 heteroatoms independently selected from O (oxygen), S (sulfur) and N (nitrogen). Exemplary heteroaryls for R¹² include, but are not limited to, furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine, pyrazine, indole, benzo[d]imidazole, pyrrolo[2,3-b]pyridine, pyrrolo[3,2-c]pyridine, pyrrolo[3,2-b]pyridine, thieno[2,3-b]pyrrole, benzofuran, benzo[b]thiophene, benzo[d]oxazole and benzo[d]thiazole. Examples of heterocyclyls for R¹² include azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, pyridin-2(1H)-one, pyridazin-3(2H)-one, morpholine, thiomorpholine, isoquinolin-1(2H)-one and 5-azaspiro[2.4]heptane. When R¹² is aryl(alkyl), heteroaryl(alkyl) or heterocyclyl(alkyl), the alkyl linker can be 1, 2 or 3 alkylene groups, such as —CH₂—, —CH₂CH₂—, —CH₂CH₂CH₂—, —CH(CH₃)—, —C(CH₃)₃— and —CH₂—(CH₃)₃—. The alkyl linker of an aryl(alkyl), a heteroaryl(alkyl) and a heterocyclyl(alkyl) can be also substituted. Possible substituents that take the place of one or more of the hydrogens (such as 1, 2, 3 or 4 hydrogens) of the alkyl linker include, but are not limited to, halogen, hydroxy and cyclopropyl (for example, —CH₂-(cyclopropyl)), or two hydrogen on the same carbon can be replaced with a spiro-connected monocyclic C₃₋₄ cycloalkyl (for example,

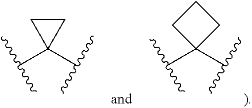 [SMILES: *C1(*)CC1 ; *C1(*)CCC1]

As provided herein, R¹² can be substituted. For example, R¹² can be substituted 1, 2, 3 or 4 times with a substituent independently selected from halogen, hydroxy, an unsubstituted C₁₋₄ alkyl, an unsubstituted C₁₋₄ alkoxy (for example —O(C₁₋₄ alkyl), such as methoxy, ethoxy, n-propoxy, iso-propoxy, n-butoxy, iso-butoxy, sec-butoxy and tert-butoxy), an unsubstituted C₁₋₄ haloalkyl, —C(═O)(an unsubstituted C₁₋₄ alkyl), —C(═O)(an unsubstituted C₁₋₄ haloalkyl), an unsubstituted monocyclic C₃₋₆ cycloalkyl, an unsubstituted phenyl, a substituted phenyl, an unsubstituted 5- or 6-membered heteroaryl, a substituted 5- or 6-membered heteroaryl, an unsubstituted 5- or 6-membered heterocyclyl and a substituted 5- or 6-membered heterocyclyl (for example a substituted phenyl, as substituted 5- or 6-membered heteroaryl and/or a substituted 5- or 6-membered heterocyclyl, which can be substituted 1, 2, 3, 4 or 5 times with a substituent selected from halogen (for example, F, Cl and Br), an unsubstituted C₁₋₄ alkyl (for example, methyl, ethyl, n-propyl, iso-propyl, n-butyl, iso-butyl, sec-butyl and tert-butyl), an unsubstituted C₁₋₄ alkoxy (for example —O(C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted —O(an unsubstituted C₁₋₄ haloalkyl) (for example, —OCF₃, —OCHF₂, —OC(CH₃)F₂, —OCH₂F, —OCH(CH₃)F, —OCH₂CF₃, —OCH₂CH₂F and —OCH₂CH₂CH₂F) and —S(═O)₂(an unsubstituted C₁₋₄ alkyl). The alkyl linker of an aryl(alkyl), a heteroaryl(alkyl) and a heterocyclyl(alkyl) can be also substituted. Possible substituents that take the place of one or more of the hydrogens (such as 1, 2, 3 or 4 hydrogens) include, but are not limited to, halogen and hydroxy. The 5- or 6-membered heteroaryl and 5- or 6-membered heterocyclyl can include 1, 2 or 3 heteroatoms selected from O (oxygen), S (sulfur) and N (nitrogen).

In some embodiments, R¹² can be an unsubstituted C-carboxy. In other embodiments, R¹² can be a substituted C-carboxy. In still other embodiments, R¹² can be an alkoxy. For example, in some embodiments, R¹² can be —OR¹³, wherein R¹³ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl). In still other embodiments, R¹² can be amino, mono-substituted amine or a di-substituted amine. In some embodiments, the amine can be —NR¹⁴R¹⁵, wherein R¹⁴ and R¹⁵ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl). In yet still other embodiments, R¹² can be C-amido. In some embodiments, R¹² can be —C(═O)—NR^{16A}R^{16B}, wherein R^{16A} can be hydrogen or an unsubstituted C₁₋₃ alkyl; and R^{16B} can be an unsubstituted or a substituted aryl, an unsubstituted or a substituted heteroaryl or an unsubstituted or a substituted 3- to 8-membered monocyclic heterocyclyl. In some embodiments, R^{16A} can be hydrogen; and R^{16B} can be an unsubstituted or a substituted phenyl, an unsubstituted or a substituted monocyclic 5- or 6-membered heteroaryl or an unsubstituted or a substituted 3- to 8-membered monocyclic heterocyclyl.

Various examples of R¹² groups include the following:

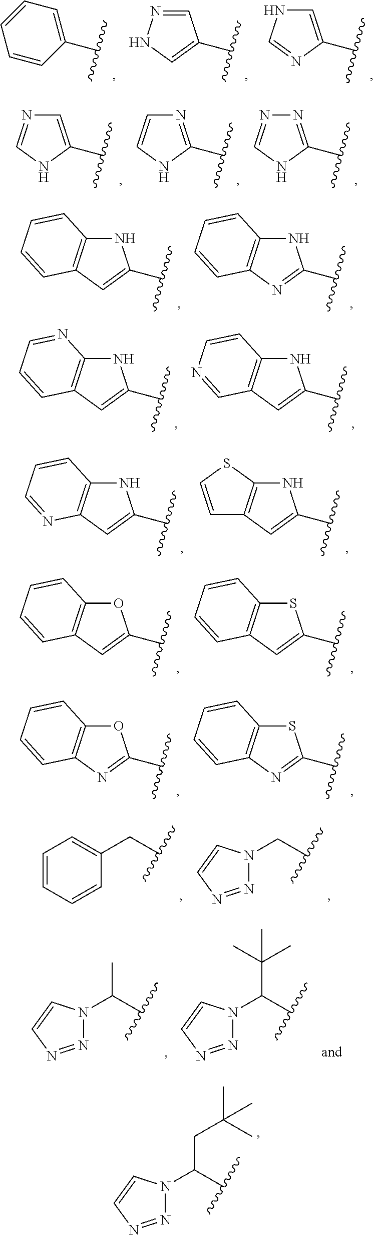

wherein each is unsubstituted or substituted (including the nitrogen). Other examples of R¹² groups include the following:

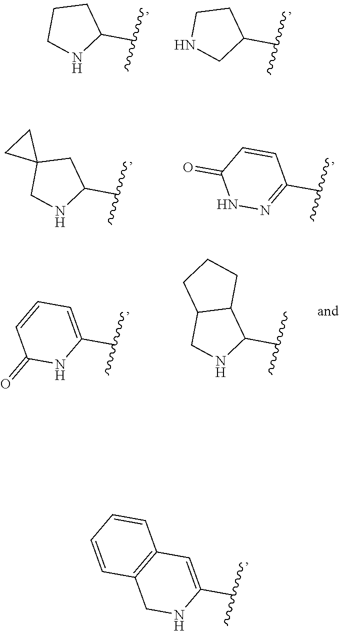

wherein each is unsubstituted or substituted (including the nitrogen). When R¹² is substituted, R¹² can be substituted with a variety of substituents. For example, R¹² can be substituted 1, 2, 3, or more than 3 times with a substituent independently selected from halogen, (such as F and C₁), an unsubstituted C₁₋₄ alkyl, an unsubstituted C₁₋₄ haloalkyl, hydroxy, an unsubstituted C₁₋₄ alkoxy, —C(═O)(an unsubstituted C₁₋₄ alkyl), —C(═O)(an unsubstituted C₁₋₄ haloalkyl) and an unsubstituted or a substituted phenyl (for example, an unsubstituted phenyl or a phenyl substituted with 1, 2 or 3 substituents independently selected from halogen, an unsubstituted C₁₋₄ alkyl, an unsubstituted C₁₋₄ haloalkyl and an unsubstituted C₁₋₄ alkoxy).

Examples of substituted R¹² groups include the following:

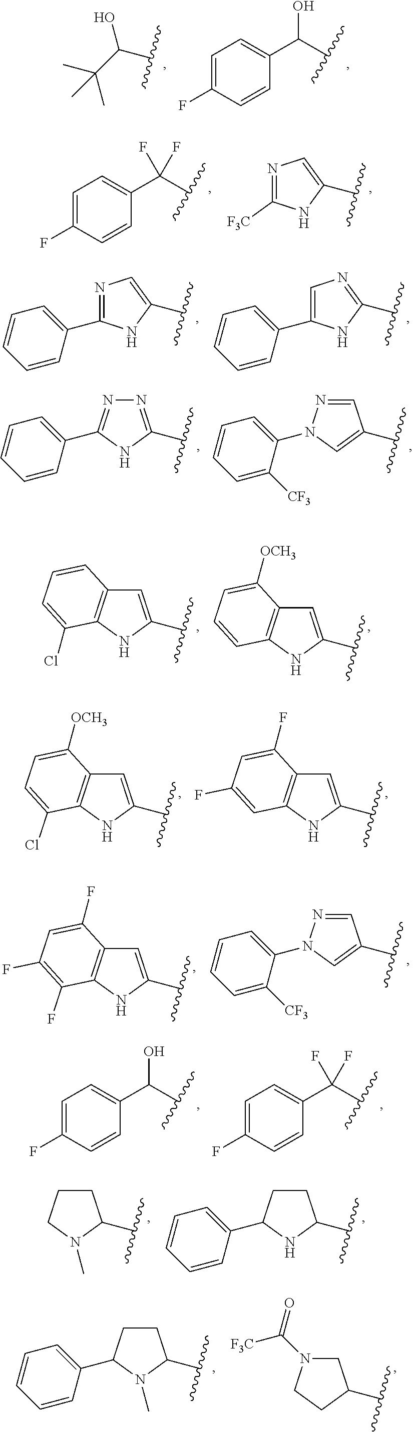

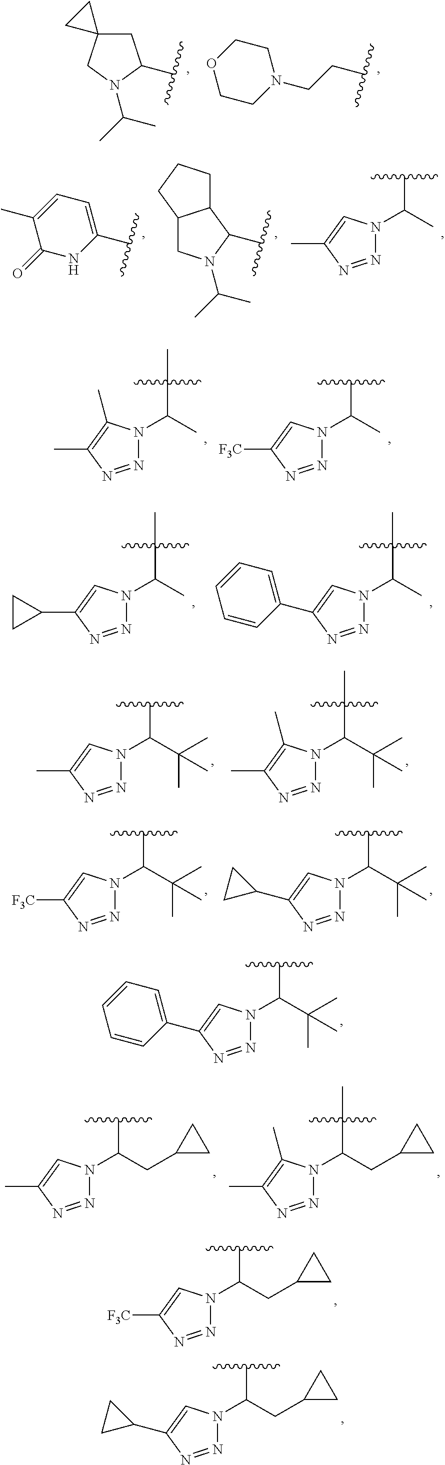

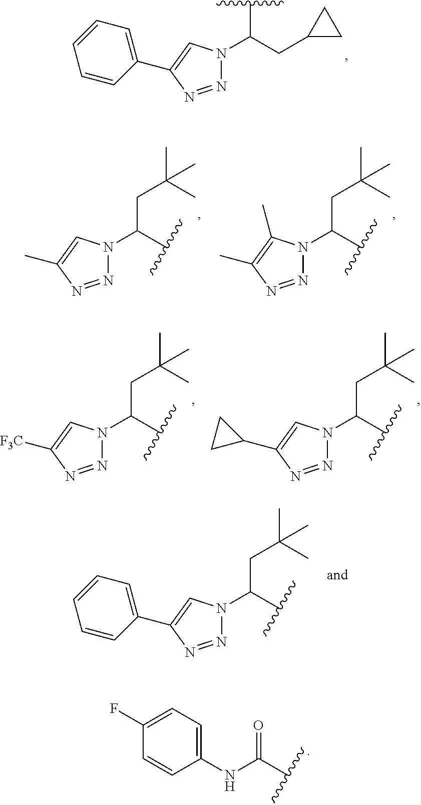

Further examples of R¹² include the following:

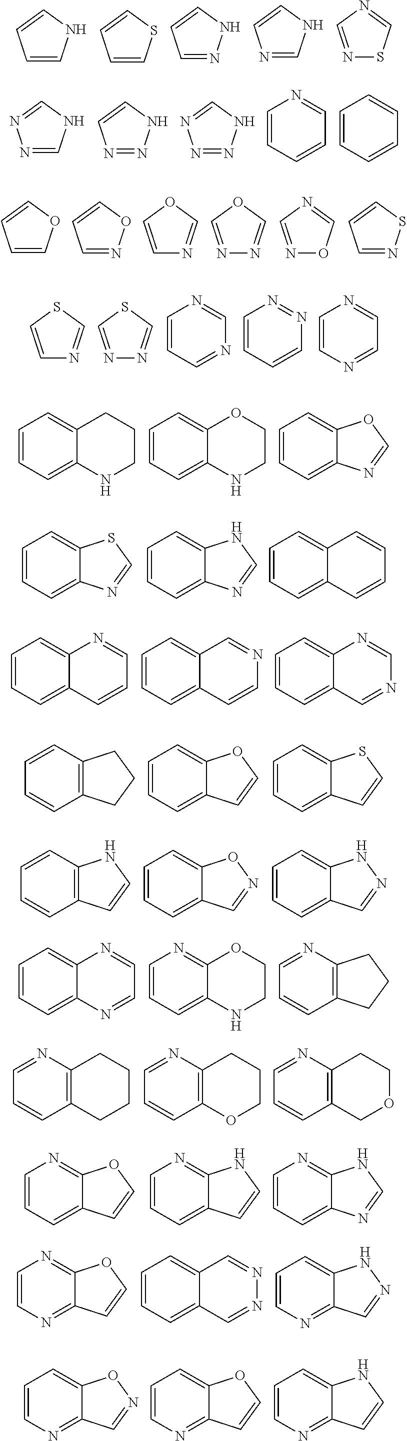

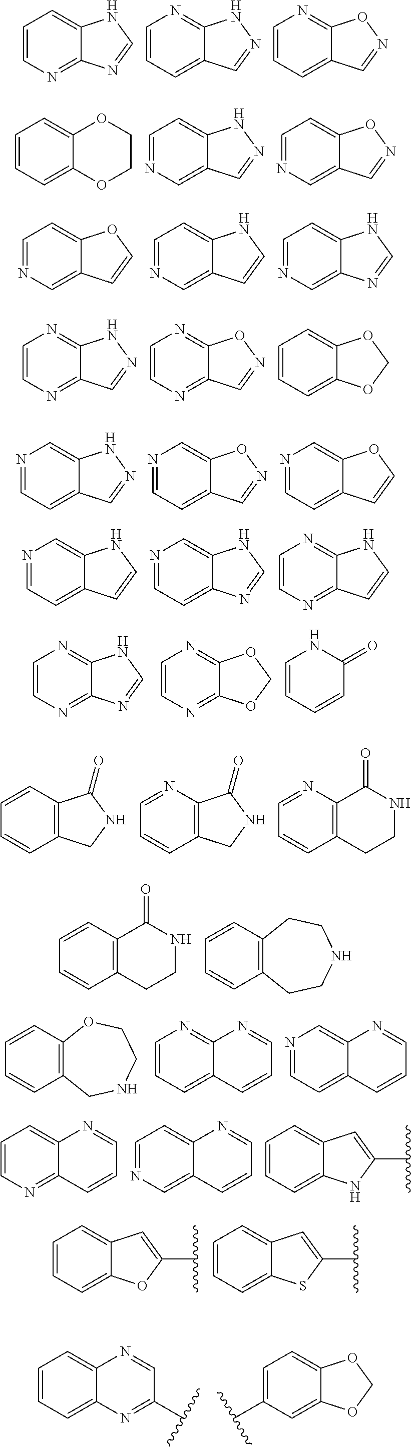

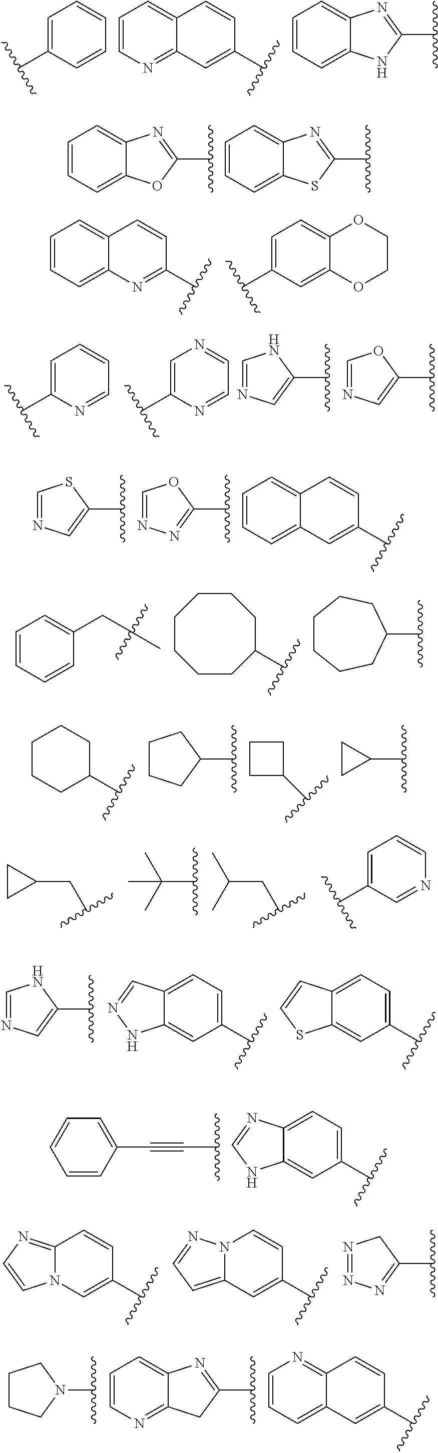

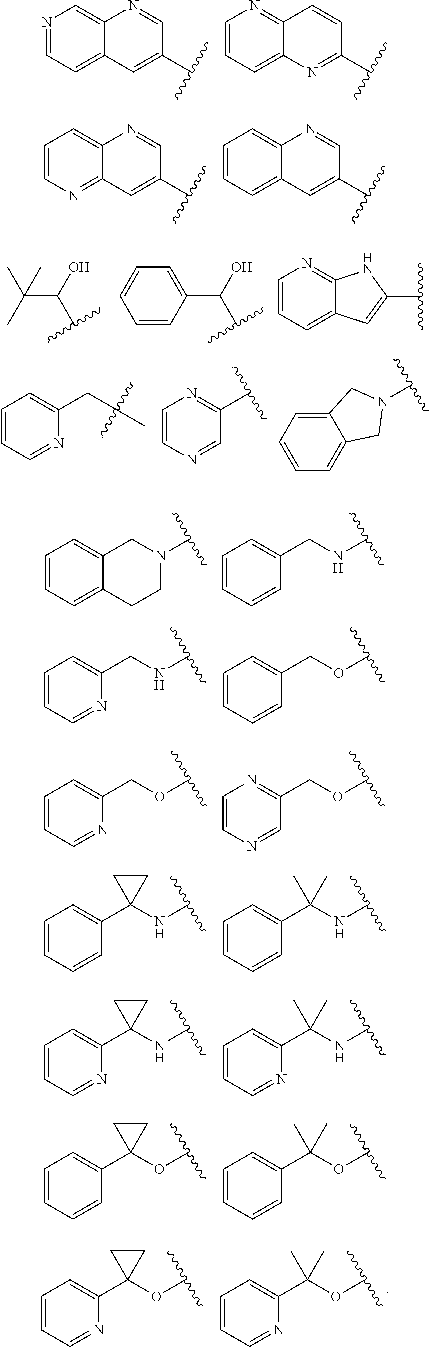

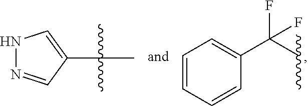 [SMILES: *c1cn[nH]c1 ; *C(F)(F)c1ccccc1]

wherein each is unsubstituted or substituted.

Exemplary R³ groups include the following:

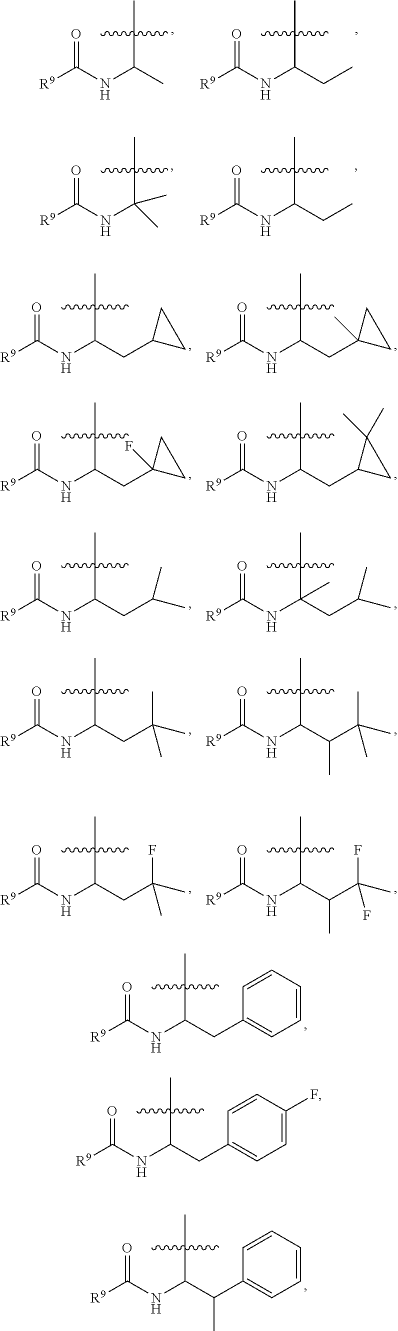

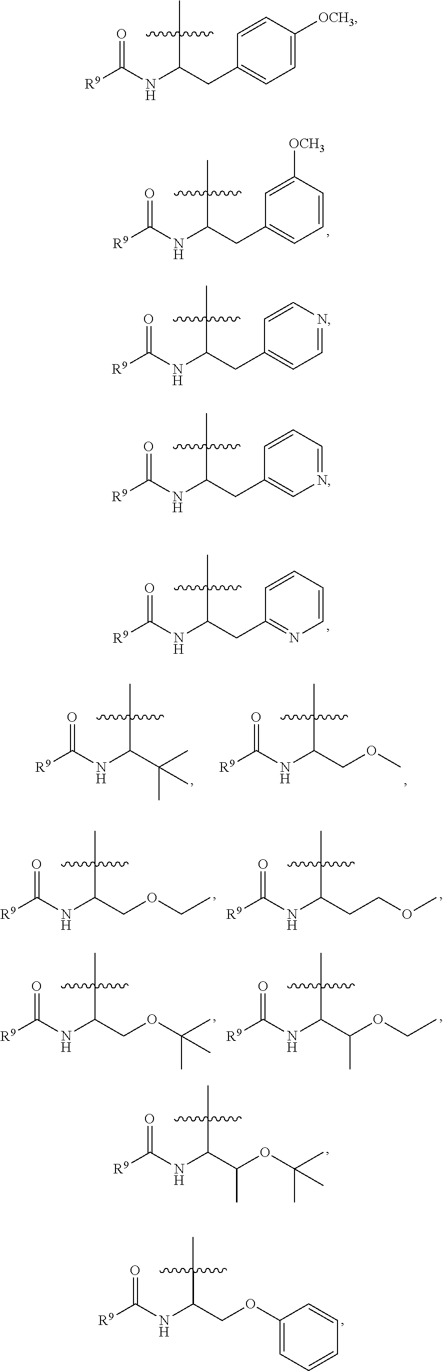

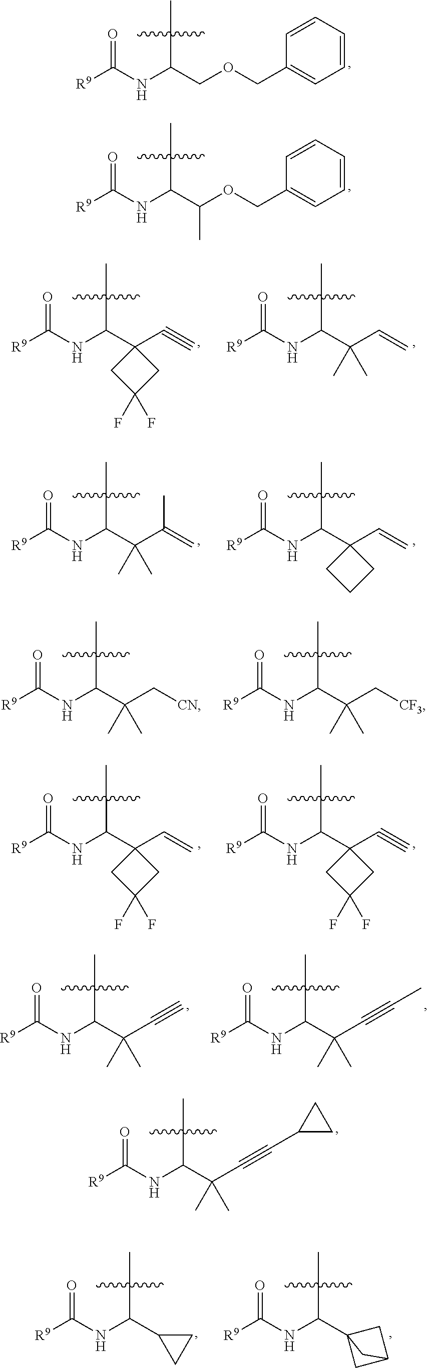

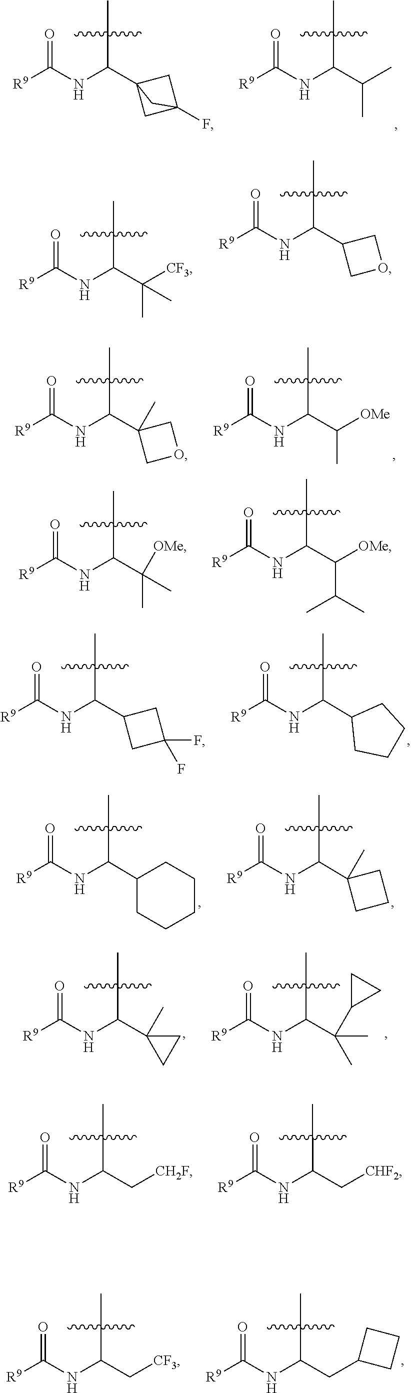

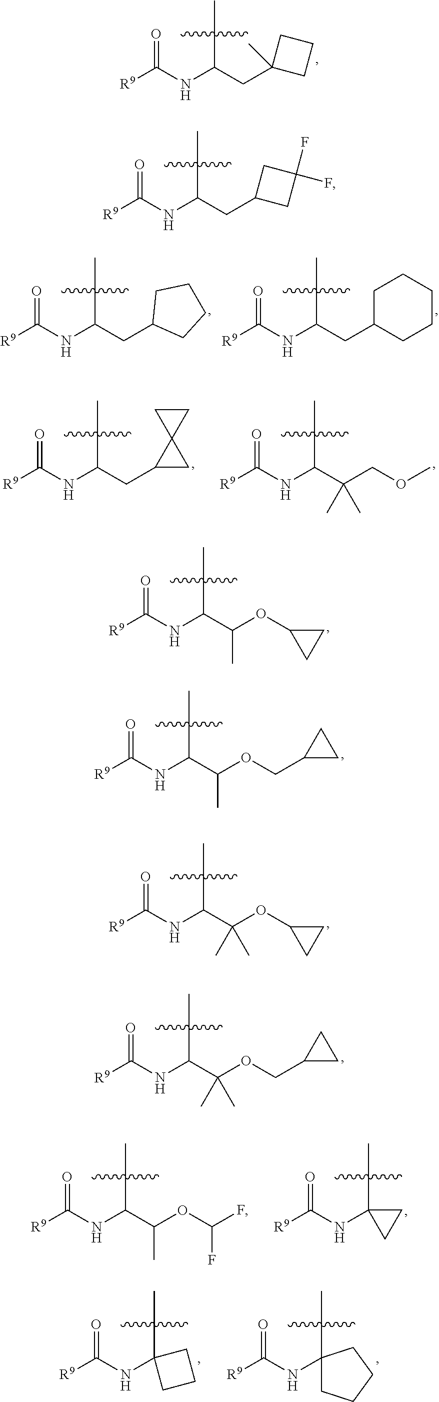

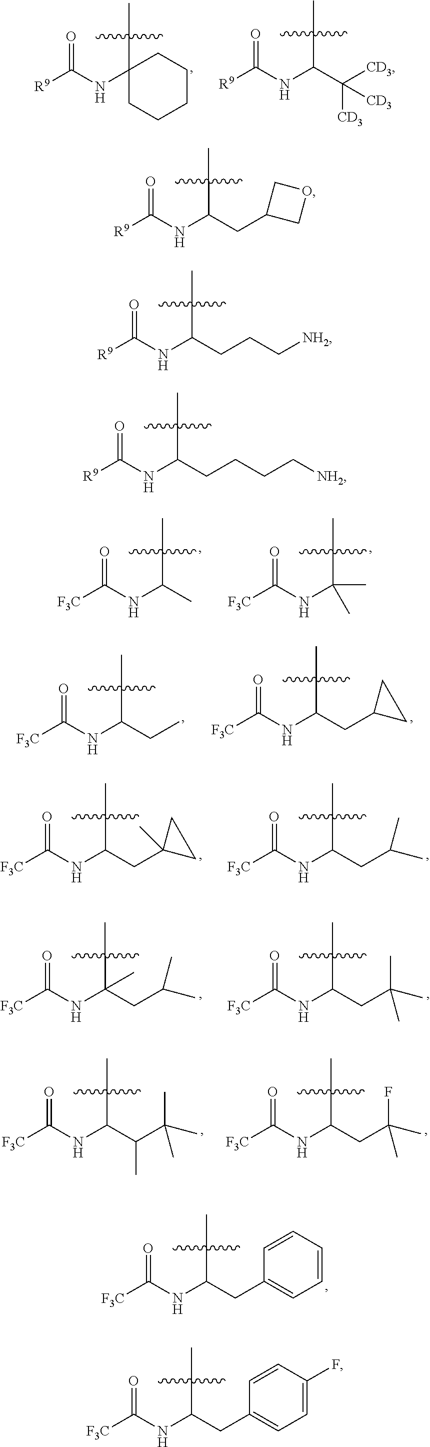

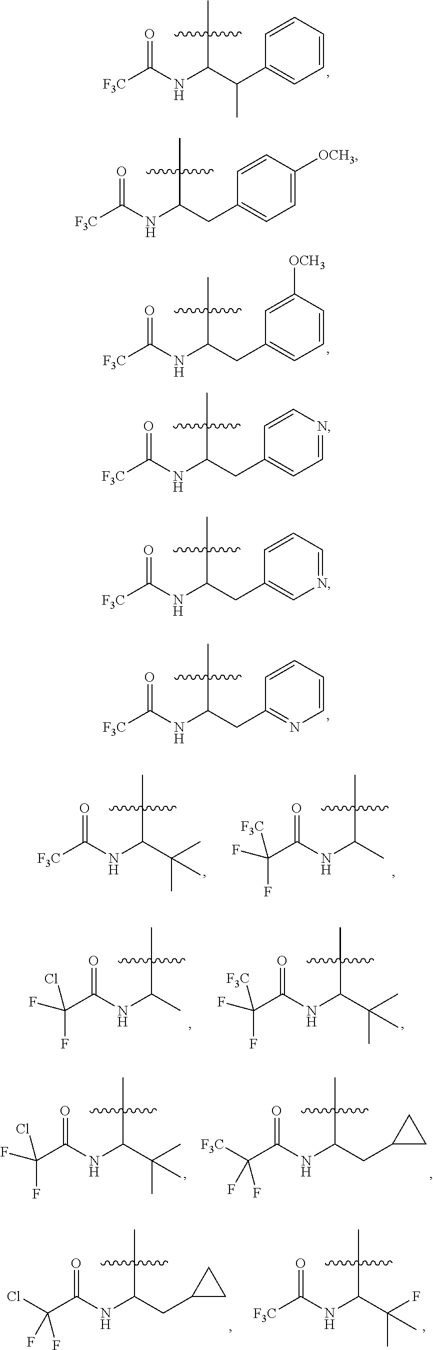

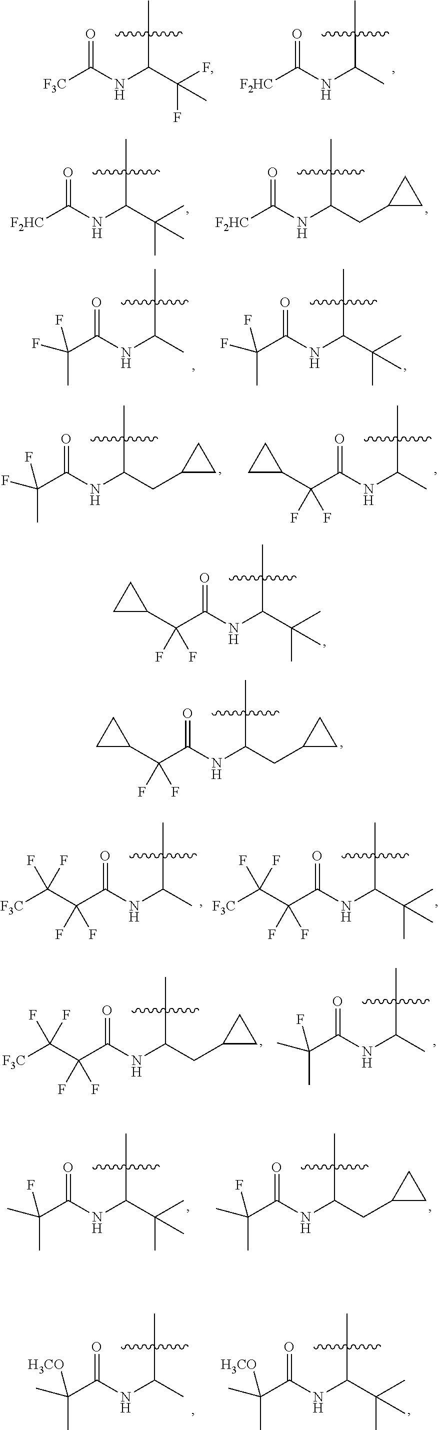

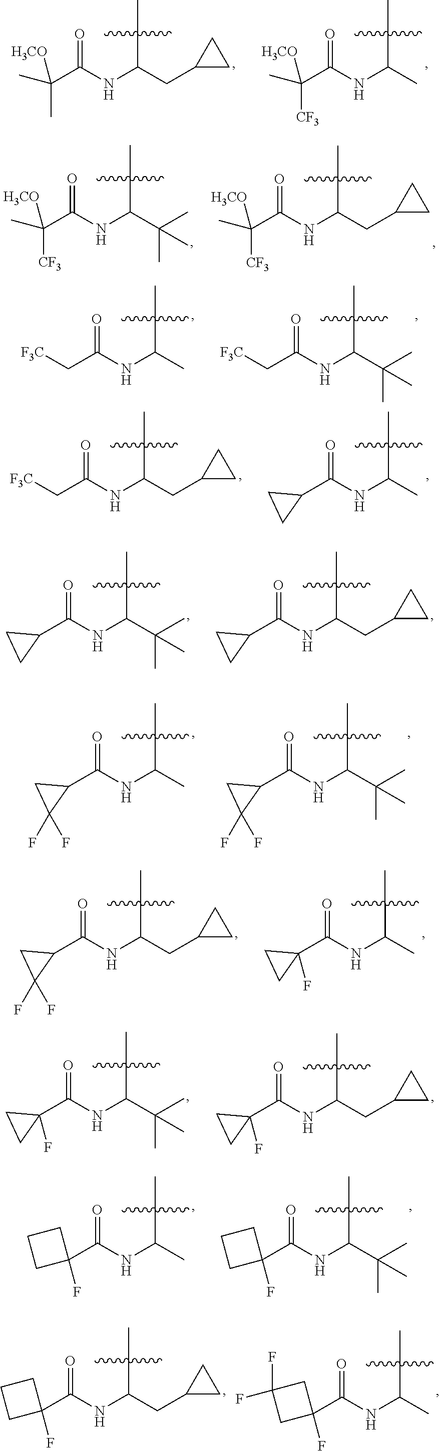

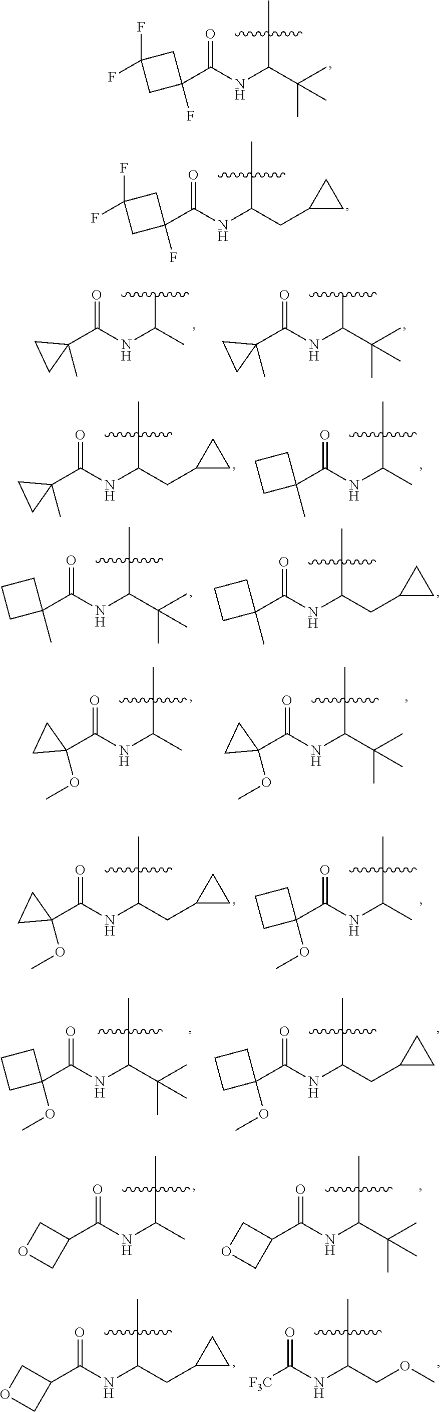

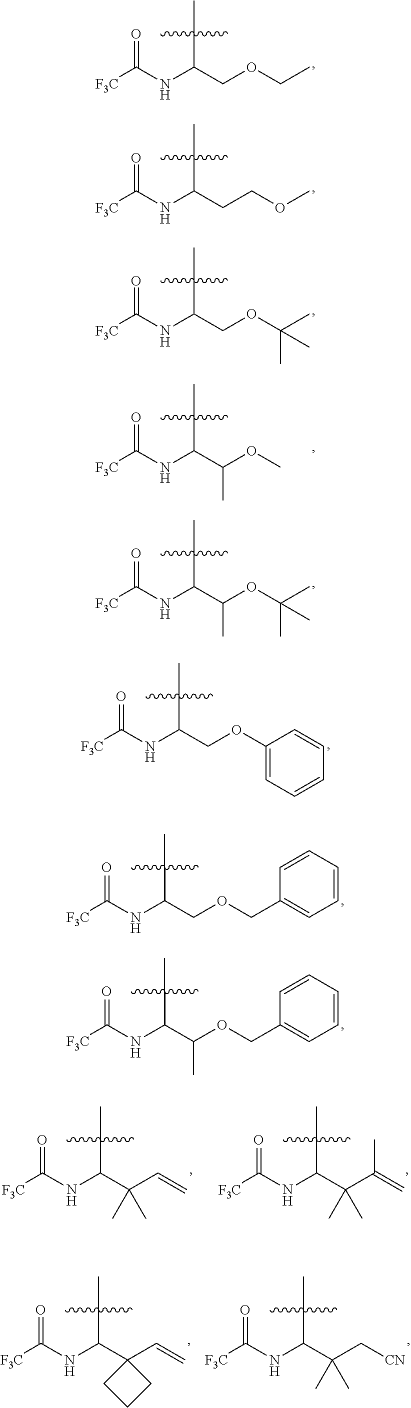

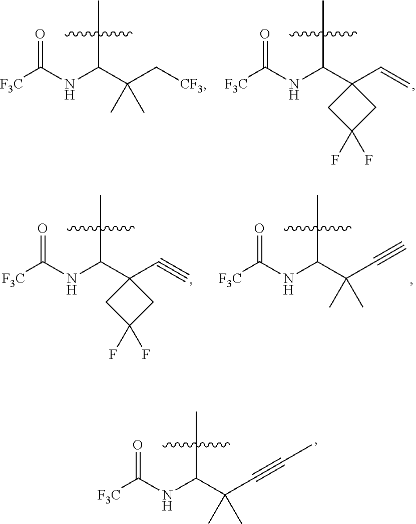

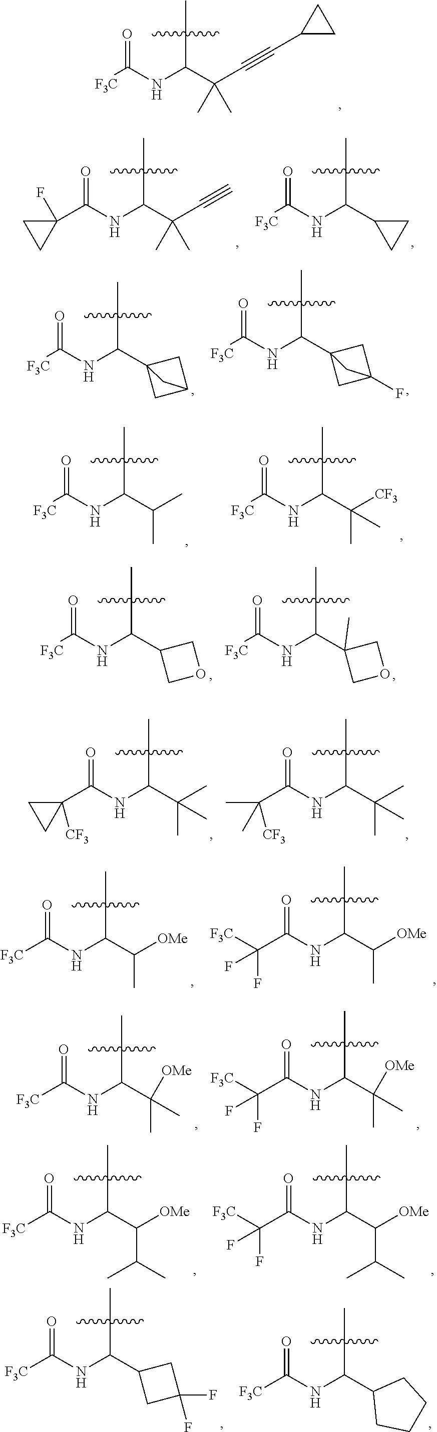

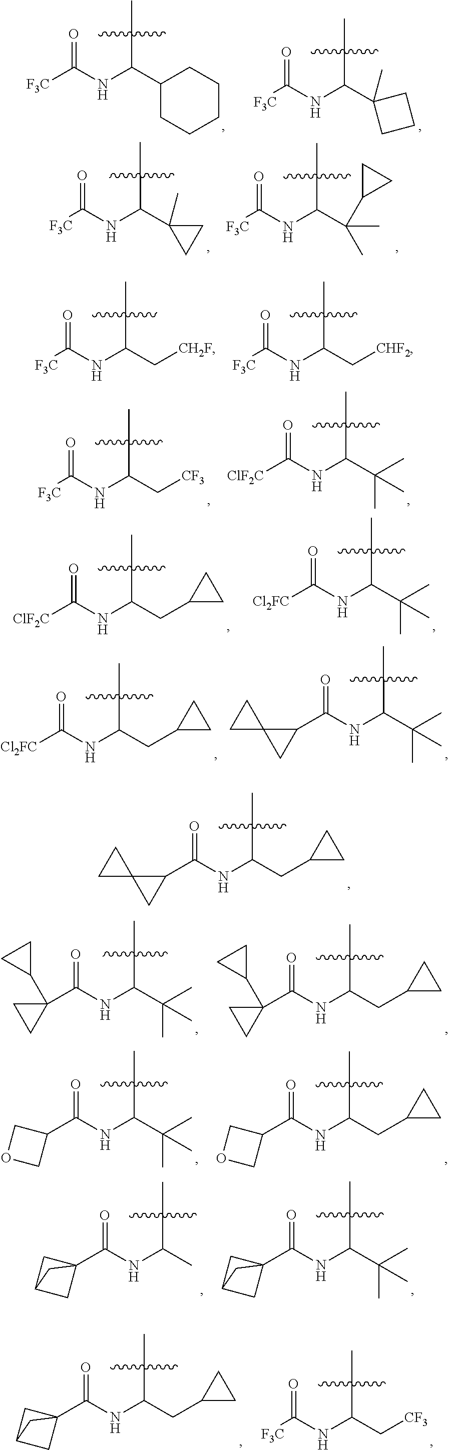

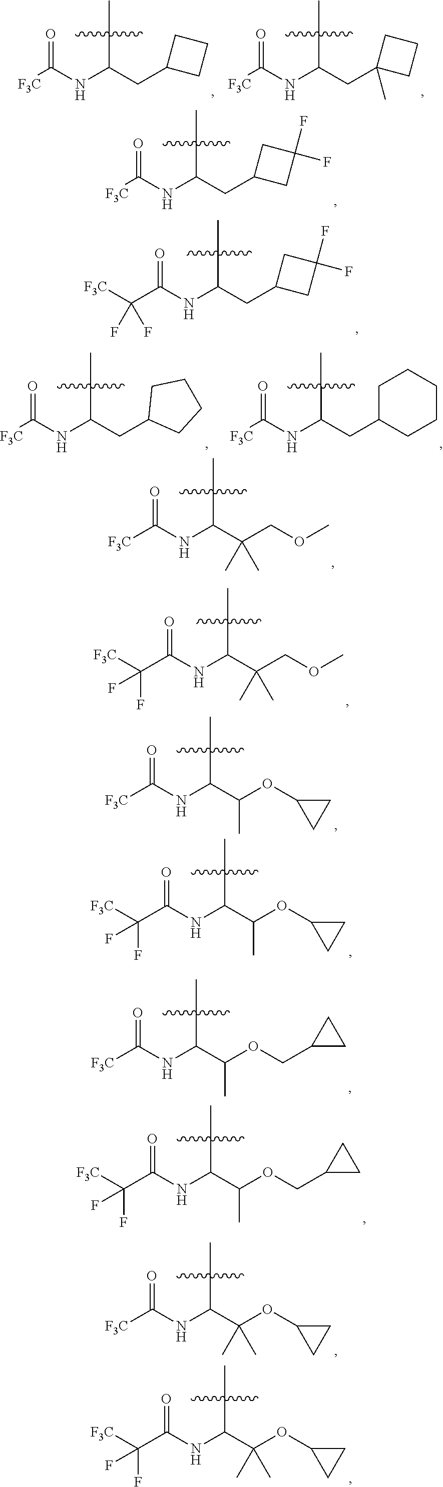

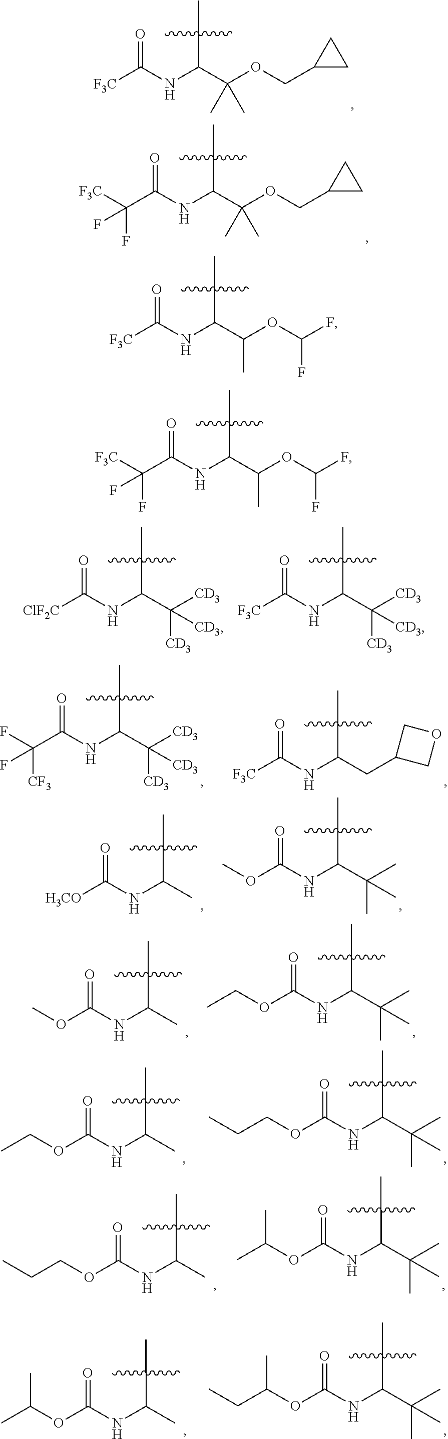

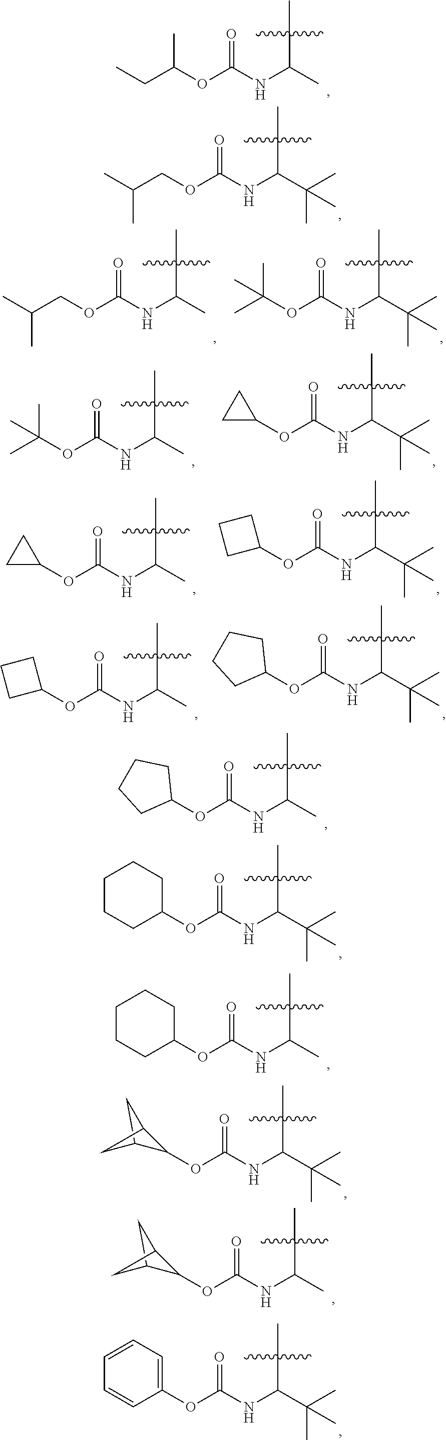

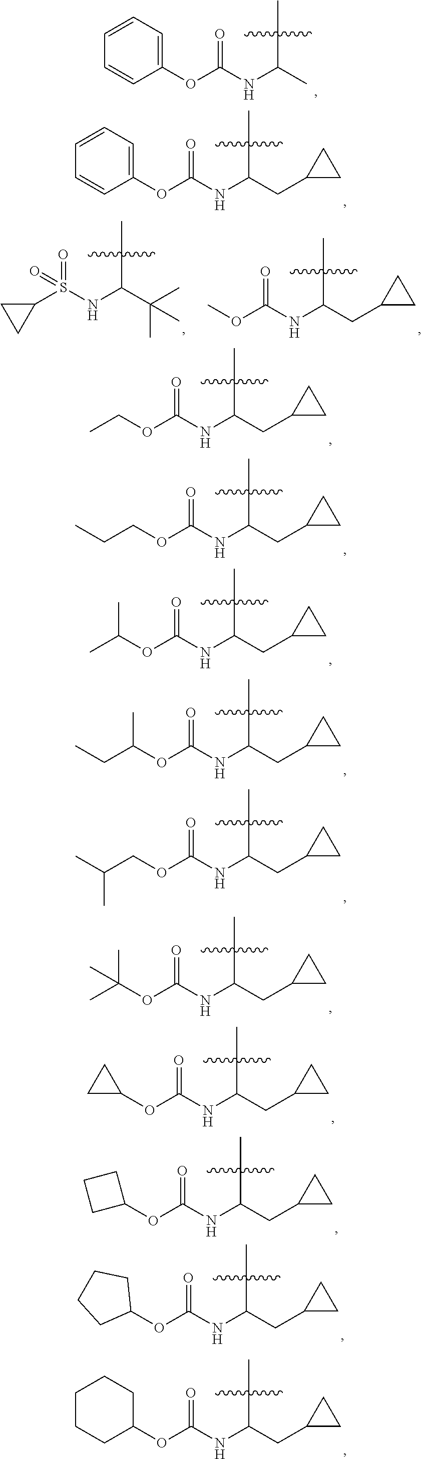

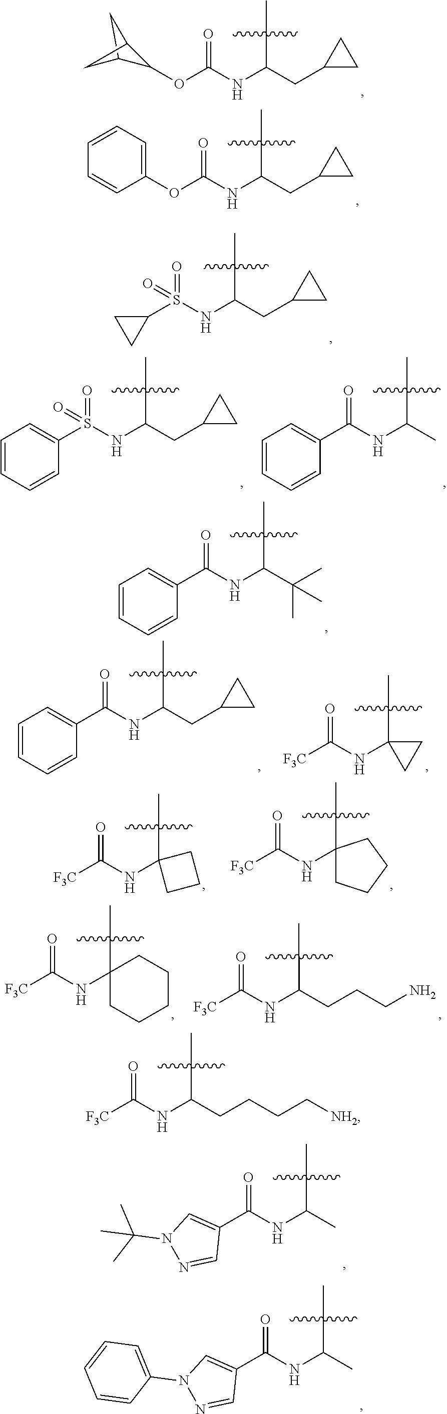

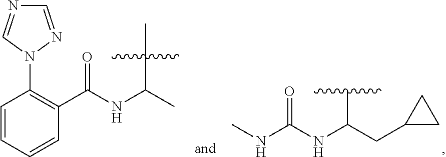 [SMILES: *C(C)NC(=O)c1ccccc1-n1cncn1 ; *C(CC1CC1)NC(=O)NC]

wherein each phenyl and can be substituted or unsubstituted as described herein. As examples, the phenyl can be substituted with 1, 2 or 3 substituents independently selected from halogen for example, fluoro and/or chloro, an unsubstituted C₁₋₄ alkyl, an unsubstituted C₁₋₄ haloalkyl and an unsubstituted C₁₋₄ alkoxy. Examples of substituted phenyls for R⁹ include

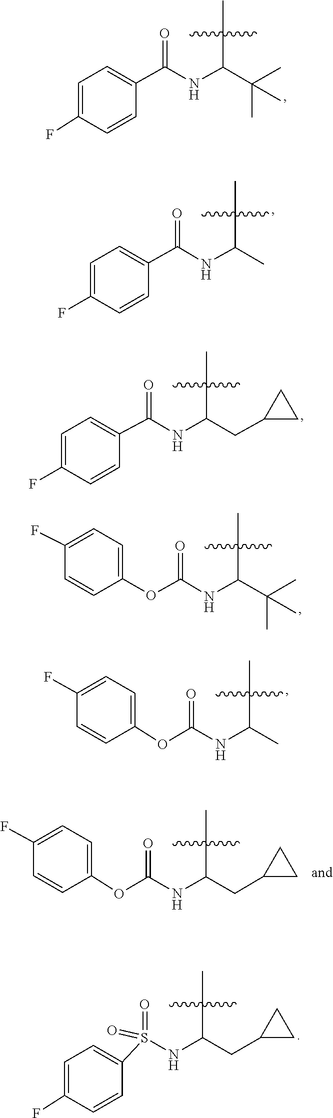

Further examples of R³ groups include:

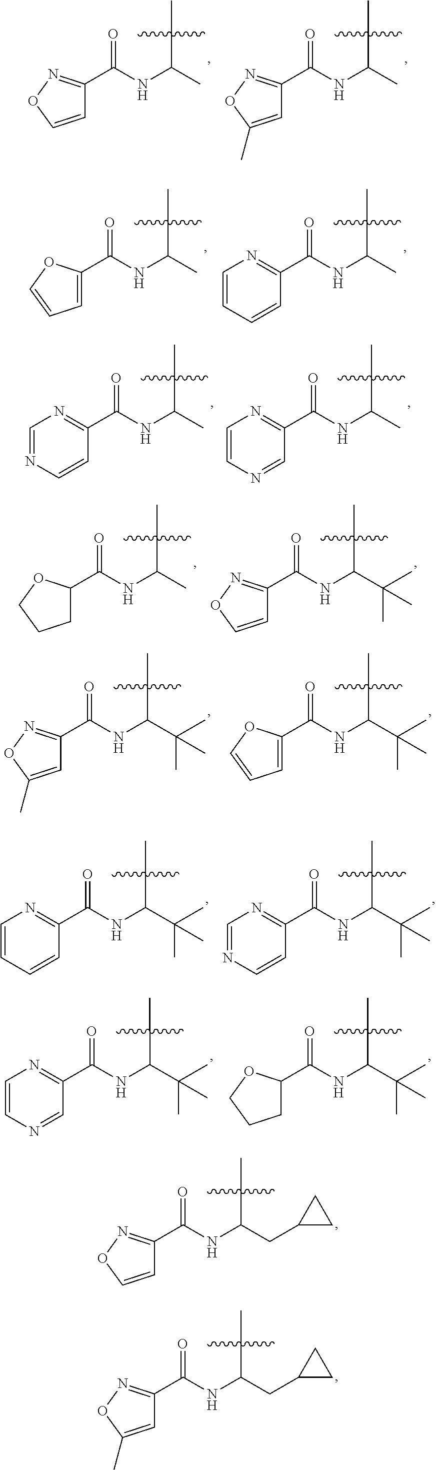

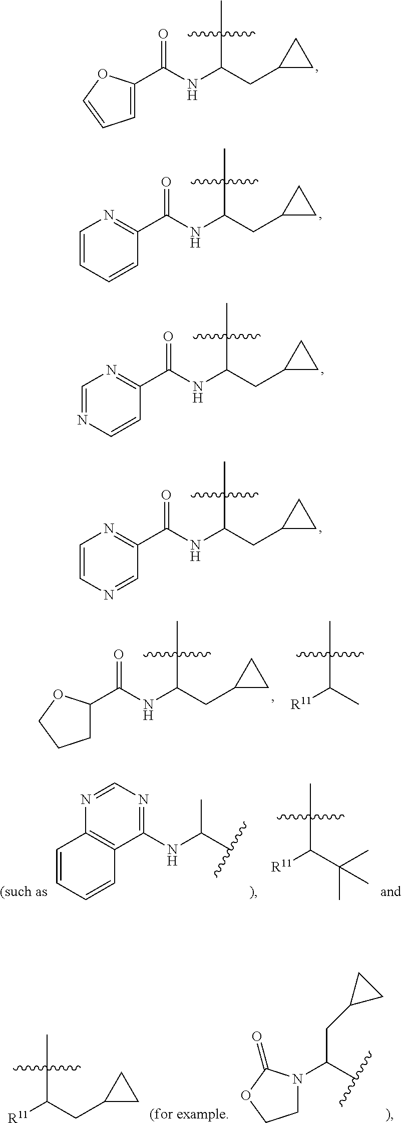

wherein each moiety is unsubstituted or substituted.

In some embodiments, R³ can be selected from

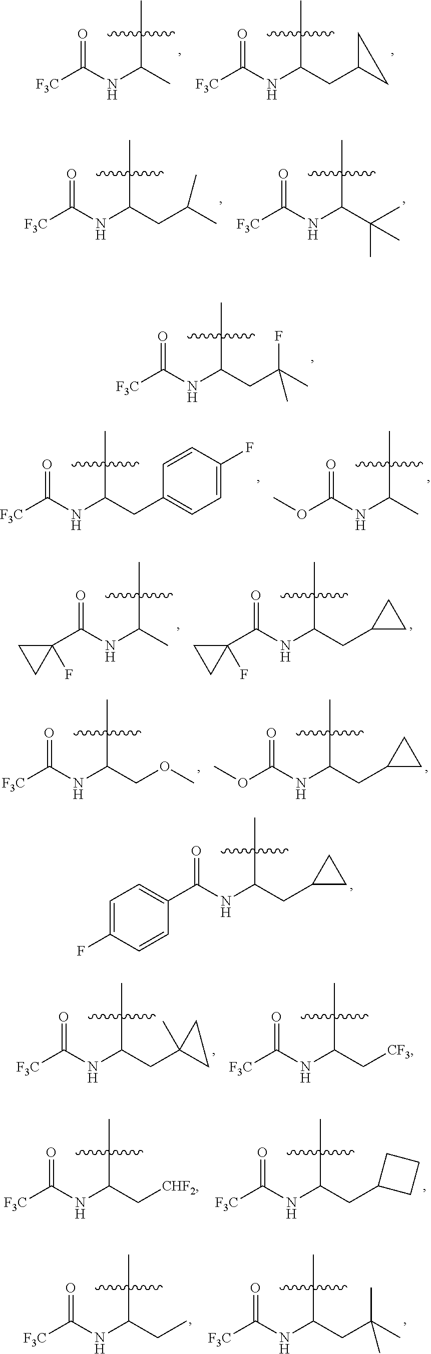

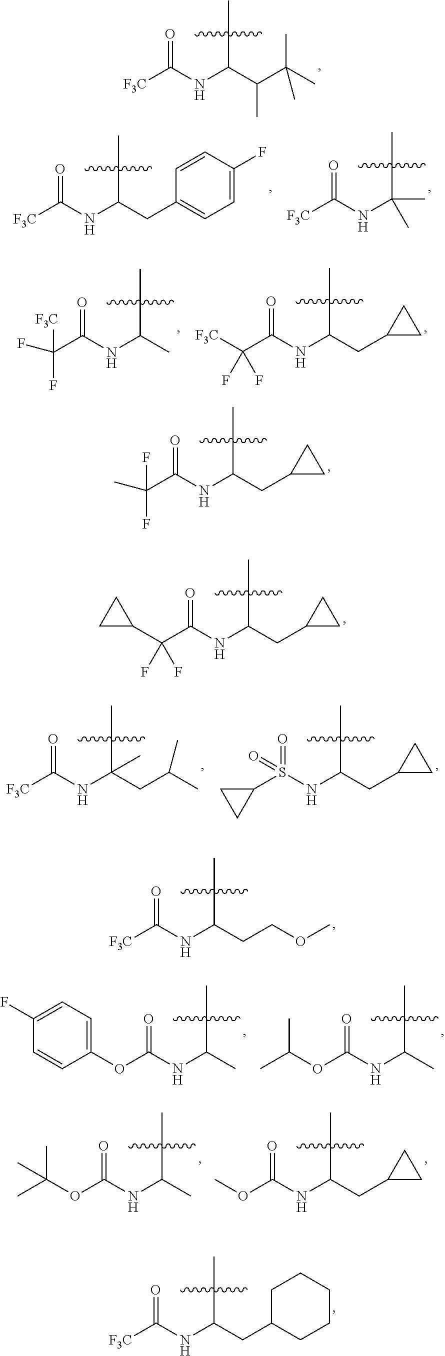

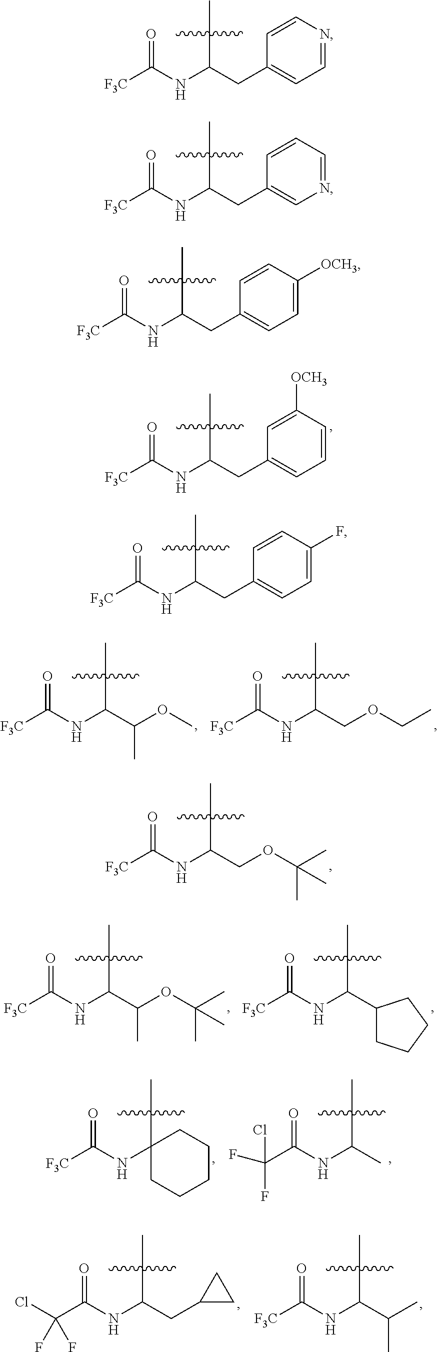

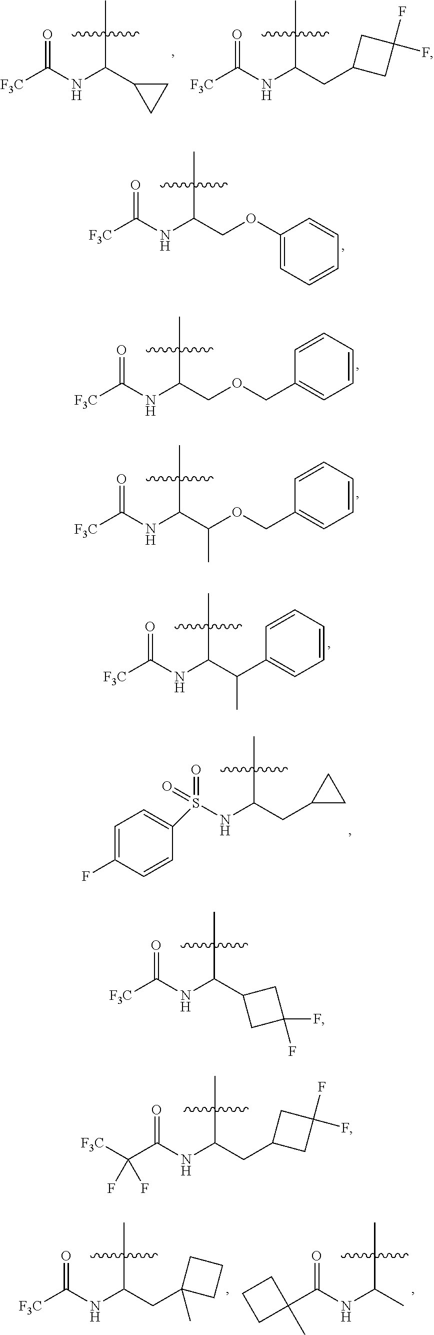

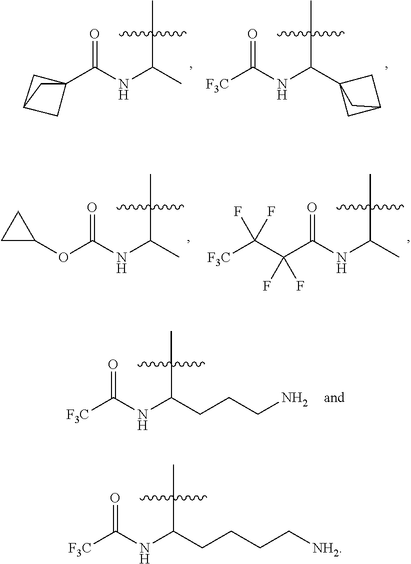

In other embodiments, R³ can be selected from

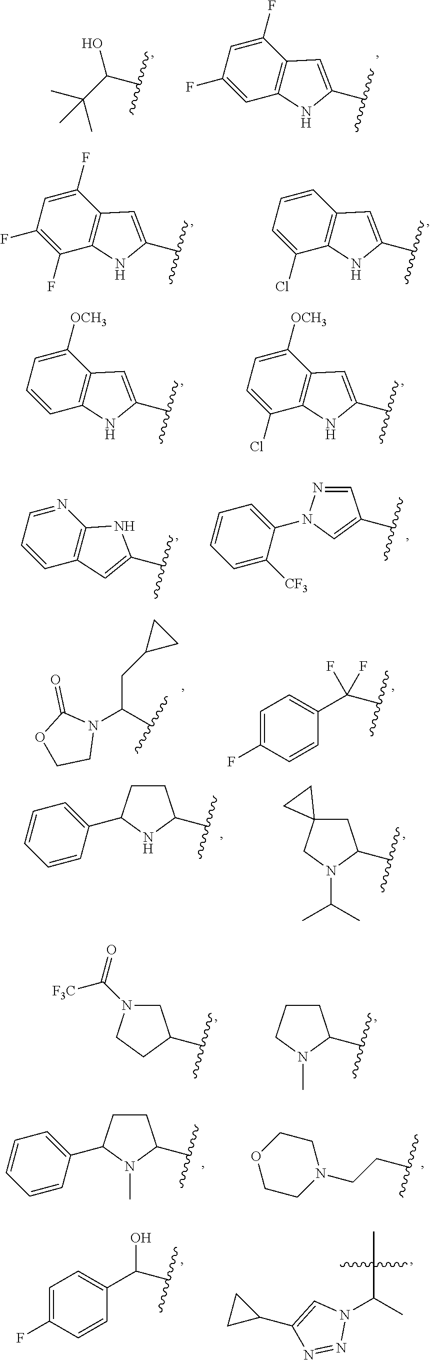

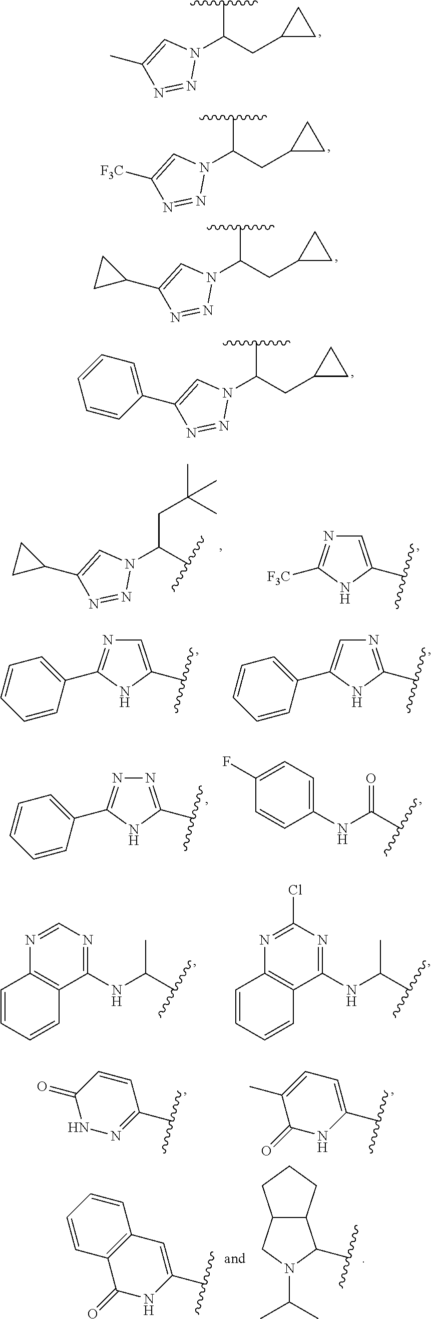

Some examples of a compound of Formula (I) (including pharmaceutically acceptable salts thereof) can be selected from:

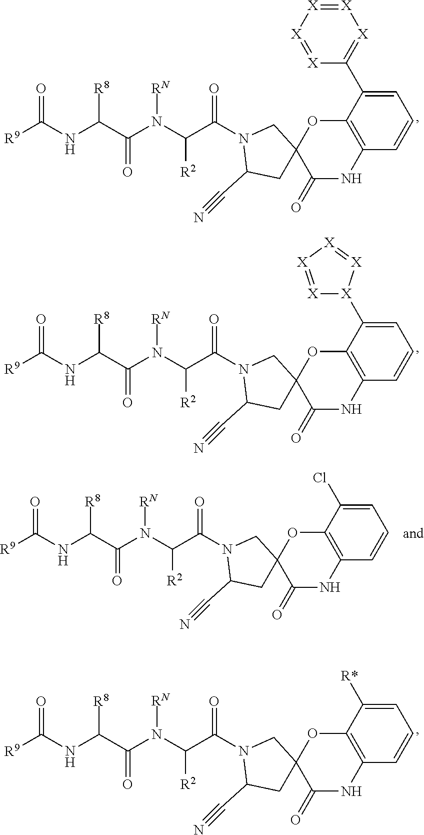

wherein each X can be independently CR** or N (nitrogen), wherein each R** can be independently selected from hydrogen, halogen, an unsubstituted C₁₋₄ alkyl, an unsubstituted monocyclic C₃₋₆ cycloalkyl, an unsubstituted —O(an unsubstituted C₁₋₄ alkyl), an unsubstituted —O(an unsubstituted C₁₋₄ haloalkyl) and —S(═O)₂(an unsubstituted C₁₋₄ alkyl); and each R* can be independently an unsubstituted C₁₋₄ alkyl or an unsubstituted —O(an unsubstituted C₁₋₄ alkyl).

In some embodiments, a compound of Formula (I), or a pharmaceutically acceptable salt thereof, can be where R^{N} can be hydrogen, deuterium or an optionally substituted C₁₋₆ alkyl; R¹ can be

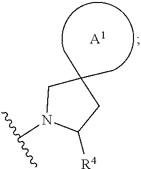

wherein Ring A¹ can be a 5-7 membered monocyclic heterocyclyl:
i) includes NR^{5a} in the ring,
ii) can be substituted with a first ═O on a carbon of the ring,
iii) optionally includes 1-3 heteroatoms selected from O (oxygen), N (nitrogen) and NR^{5b} in the ring of Ring A¹,
iv) can be optionally substituted with one or more moieties selected from a second ═O on a ring carbon, halogen, an unsubstituted C₁₋₆ alkyl, an optionally substituted C₃₋₆ cycloalkyl and an optionally substituted phenyl, and
v) can be optionally fused to an optionally substituted phenyl, an optionally substituted monocyclic heteroaryl or an optionally substituted bicyclo[4.2.0]octa-1(6),2,4-triene;
vi) provided that when R¹ is

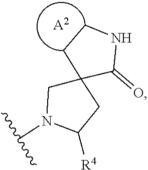

then Ring A² cannot be an optionally substituted phenyl or an optionally substituted monocyclic heteroaryl; R² can be hydrogen, an optionally substituted C₁₋₈ alkyl, an optionally substituted C₂₋₈ alkenyl, an optionally substituted C₂₋₈ alkynyl, an optionally substituted C₃₋₁₀ cycloalkyl, an optionally substituted C₃₋₁₀ cycloalkenyl, an optionally substituted aryl, an optionally substituted aryl(alkyl), an optionally substituted heteroaryl, an optionally substituted heteroaryl(alkyl), an optionally substituted heterocyclyl or an optionally substituted heterocyclyl(alkyl); R³ can be

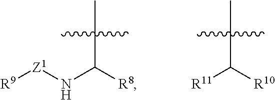

or R¹²; Z¹ can be —C(═O)— or —S(═O)₂—; R⁴ can be selected from cyano, an unsubstituted or a substituted C₂₋₅ alkynyl, an unsubstituted or a substituted acyl, an unsubstituted or a substituted ketoamide, —CH(OH)—(S(═O)₂—OH), —CH(OH)—(S(═O)₂—O—), —CH(OH)((P═O)(OR⁶)₂) and —C(═O)CH₂—O—((P═O)(OR⁷)₂); R^{5a} can be selected from hydrogen, an optionally substituted C₁₋₄ alkyl, an optionally substituted C₂₋₄ alkenyl and an optionally substituted C₃₋₆ cycloalkyl; R^{5b} can be selected from hydrogen, an optionally substituted C₁₋₄ alkyl, an optionally substituted C₂₋₄ alkenyl and an optionally substituted C₃₋₆ cycloalkyl; each R⁶ and each R⁷ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl); R⁸ and R¹⁰ can be independently selected from an unsubstituted or a substituted C₁₋₆ alkyl, an unsubstituted or a substituted C₂₋₆ alkenyl, an unsubstituted or a substituted C₂₋₆ alkynyl, an unsubstituted or a substituted monocyclic C₃₋₆ cycloalkyl, an unsubstituted or a substituted bicyclic C₅₋₈ cycloalkyl, an unsubstituted or a substituted monocyclic 4- to 6-membered heterocyclyl and an unsubstituted monocyclic C₃₋₆ cycloalkyl(CH₂)—, wherein when the C₁₋₆ alkyl is substituted, the C₁₋₆ alkyl can be substituted 1, 2, 3 or 4 times with a substituent independently selected from halogen, cyano, an unsubstituted or a substituted monocyclic C₃₋₆ cycloalkyl, an unsubstituted C₁₋₄ alkoxy and an unsubstituted C₁₋₄ haloalkoxy, or the C₁₋₆ alkyl is substituted 1 to 13 times with deuterium; wherein when the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted, the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl can be substituted 1, 2, 3 or 4 times with a substituent independently selected from halogen, an unsubstituted C₁₋₄ alkyl, an unsubstituted C₂₋₄ alkenyl, an unsubstituted C₂₋₄ alkynyl, an unsubstituted C₁₋₄ haloalkyl, an unsubstituted or a substituted monocyclic C₃₋₆ cycloalkyl and an unsubstituted C₁₋₄ alkoxy; and R⁹ can be selected from an unsubstituted or a substituted C₁₋₆ alkyl, an unsubstituted or a substituted C₁₋₆ haloalkyl, an unsubstituted or a substituted monocyclic C₃₋₆ cycloalkyl, an unsubstituted or a substituted bicyclic C₅₋₆ cycloalkyl, an unsubstituted or a substituted monocyclic heteroaryl, an unsubstituted or a substituted monocyclic heterocyclyl, an unsubstituted or a substituted alkoxy and —NR¹⁷R¹⁸, wherein the substituted C₁₋₆ alkyl can be substituted 1 or 2 times with an unsubstituted C₁₋₄ alkoxy, wherein the substituted monocyclic C₃₋₆ cycloalkyl can be substituted 1, 2, 3 or 4 times with a substituent independently selected from halogen, an unsubstituted C₁₋₄ alkyl, an unsubstituted C₁₋₄ alkoxy, an unsubstituted C₁₋₄ haloalkyl and an unsubstituted monocyclic C₃₋₆ cycloalkyl, and wherein the substituted C₁₋₆ haloalkyl can be substituted 1 or 2 times with an unsubstituted C₁₋₄ alkoxy; R″ can be an optionally substituted monocyclic 4- to 6-membered heterocyclyl, —(NH)ₘ— an optionally substituted 5- to 6-membered monocyclic heteroaryl, —O— an optionally substituted C₁₋₆ alkyl, —O— an optionally substituted C₃₋₈ cycloalkyl or —O— an optionally substituted C₃₋₈ cycloalkyl(C₁₋₄ alkyl), wherein m can be 0 or 1; R¹² can be an optionally substituted C₁₋₈ alkyl, an optionally substituted C₂₋₈ alkenyl, an optionally substituted C₂₋₈ alkynyl, an optionally substituted monocyclic C₃₋₈ cycloalkyl, an optionally substituted aryl, an optionally substituted heteroaryl, an optionally substituted 3- to 8-membered monocyclic heterocyclyl, an optionally substituted aryl(alkyl), an optionally substituted heteroaryl(alkyl), an optionally substituted C-carboxy, —OR¹³, —NR¹⁴R¹⁵ or —C(═O)—NR^{16A}R^{16B}; R¹³ can be an optionally substituted C₁₋₈ alkyl, an optionally substituted C₂₋₈ alkenyl, an optionally substituted C₂₋₈ alkynyl, an optionally substituted monocyclic C₃₋₈ cycloalkyl, an optionally substituted aryl, an optionally substituted heteroaryl, an optionally substituted 3- to 8-membered monocyclic heterocyclyl, an optionally substituted aryl(alkyl) or an optionally substituted heteroaryl(alkyl); R¹⁴ are R¹⁵ can be independently selected from hydrogen, an optionally substituted C₁₋₈ alkyl, an optionally substituted C₂₋₈ alkenyl, an optionally substituted C₂₋₈ alkynyl, an optionally substituted monocyclic C₃₋₈ cycloalkyl, an optionally substituted aryl, an optionally substituted heteroaryl, an optionally substituted 3- to 8-membered monocyclic heterocyclyl, an optionally substituted aryl(alkyl) and an optionally substituted heteroaryl(alkyl); or R¹⁴ and R¹⁵ can be taken together with the nitrogen to which R¹⁴ and R¹⁵ are attached to form an optionally substituted 3- to 8-membered heterocyclyl; R^{16A} can be hydrogen or an unsubstituted C₁₋₃ alkyl; R^{16B} can be an optionally substituted aryl, an optionally substituted heteroaryl or an optionally substituted 3- to 8-membered monocyclic heterocyclyl; and R¹⁷ and R¹⁸ can be independently selected from hydrogen, an optionally substituted C₁₋₈ alkyl, an optionally substituted C₂₋₈ alkenyl, an optionally substituted C₂₋₈ alkynyl, an optionally substituted C₃₋₈ cycloalkyl, an optionally substituted 3-8 membered heterocyclyl, an optionally substituted aryl, an optionally substituted heteroaryl, an optionally substituted aryl(alkyl) and an optionally substituted heteroaryl(alkyl); or R¹⁷ and R¹⁸ can be taken together along with the nitrogen to which they are connected to form an optionally substituted 3-8 membered heterocyclyl.

In some embodiments, a compound of Formula (I), or a pharmaceutically acceptable salt thereof, can be where R^{N} can be hydrogen, deuterium or an unsubstituted or a substituted C₁₋₆ alkyl; R¹ can be

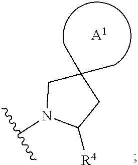

wherein Ring A¹ can be a 5-7 membered monocyclic heterocyclyl:
i) includes NR^{5a} in the ring;
ii) can be substituted with a first ═O on a carbon of the ring;
iii) optionally includes 1-3 heteroatoms selected from O (oxygen), S (sulfur), S(═O)₂, N (nitrogen) and NR^{5b} in the ring of Ring A¹;
iv) can be optionally substituted with one or more moieties selected from a second ═O on a ring carbon, halogen, hydroxy, an unsubstituted C₁₋₆ alkyl, an unsubstituted —O(C₁₋₆ alkyl), an unsubstituted or a substituted phenoxy, an unsubstituted or a substituted C₃₋₆ cycloalkyl, an unsubstituted or a substituted phenyl and an unsubstituted or a substituted benzyl;
v) can be optionally fused to an unsubstituted or a substituted phenyl, an unsubstituted or a substituted monocyclic heteroaryl, an unsubstituted or a substituted monocyclic heterocyclyl, an unsubstituted or a substituted bicyclic heteroaryl, an unsubstituted or a substituted bicyclic heterocyclyl, an unsubstituted or a substituted monocyclic cycloalkenyl or an unsubstituted or a substituted bicyclic cycloalkenyl; and
vi) provided that when R¹ is

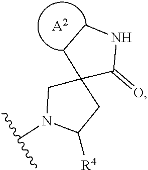

then Ring A² can be an unsubstituted or a substituted monocyclic heterocyclyl, an unsubstituted or a substituted monocyclic cycloalkenyl or an unsubstituted or a substituted bicyclic cycloalkenyl;
R² can be hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₁₀ cycloalkyl, an unsubstituted or a substituted C₃₋₁₀ cycloalkenyl, an unsubstituted or a substituted aryl, an unsubstituted or a substituted aryl(alkyl), an unsubstituted or a substituted heteroaryl, an unsubstituted or a substituted heteroaryl(alkyl), an unsubstituted or a substituted heterocyclyl or an unsubstituted or a substituted heterocyclyl(alkyl); R³ can be

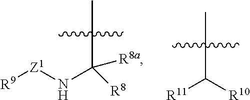

or R¹²; Z¹ can be —C(═O)— or —S(═O)₂—; R⁴ can be selected from cyano, an unsubstituted or a substituted C₂₋₅ alkynyl, an unsubstituted or a substituted acyl, an unsubstituted or a substituted ketoamide, —C(═O)NH₂, —CH(OH)—(S(═O)₂—OH), —CH(OH)—(S(═O)₂—O—), —CH(OH)((P═O)(OR⁶)₂) and —C(═O)CH₂—O—((P═O)(OR⁷)₂); R^{5a} can be selected from hydrogen, an unsubstituted or a substituted C₁₋₄ alkyl, an unsubstituted or a substituted C₂₋₄ alkenyl and an unsubstituted or a substituted C₃₋₆ cycloalkyl; R^{5b} can be selected from hydrogen, an unsubstituted or a substituted C₁₋₄ alkyl, an unsubstituted or a substituted C₂₋₄ alkenyl and an unsubstituted or a substituted C₃₋₆ cycloalkyl; each R⁶ and each R⁷ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl); R⁸ and R¹⁰ can be independently selected from an unsubstituted or a substituted C₁₋₆ alkyl, an unsubstituted or a substituted C₂₋₆ alkenyl, an unsubstituted or a substituted C₂₋₆ alkynyl, an unsubstituted or a substituted monocyclic C₃₋₆ cycloalkyl, an unsubstituted or a substituted bicyclic C₅₋₈ cycloalkyl and an unsubstituted or a substituted monocyclic 4- to 6-membered heterocyclyl, wherein when the C₁₋₆ alkyl is substituted, the C₁₋₆ alkyl can be substituted 1, 2, 3 or 4 times with a substituent independently selected from halogen, cyano, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic 4-6 membered heterocyclyl, an unsubstituted C₁₋₄ alkoxy and an unsubstituted C₁₋₄ haloalkoxy, or the C₁₋₆ alkyl is substituted 1 to 13 times with deuterium; wherein when the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted, the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl can be substituted 1, 2, 3 or 4 times with a substituent independently selected from halogen, an unsubstituted C₁₋₄ alkyl, an unsubstituted C₂₋₄ alkenyl, an unsubstituted C₂₋₄ alkynyl, an unsubstituted C₁₋₄ haloalkyl, an unsubstituted or a substituted monocyclic C₃₋₆ cycloalkyl and an unsubstituted C₁₋₄ alkoxy; and R^{8a} can be hydrogen or an unsubstituted C₁₋₄ alkyl; R⁹ can be selected from an unsubstituted or a substituted C₁₋₆ alkyl, an unsubstituted or a substituted C₁₋₆ haloalkyl, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic heteroaryl, an unsubstituted or a substituted monocyclic heterocyclyl, an unsubstituted or a substituted alkoxy and —NR¹⁷R¹⁸, wherein the substituted C₁₋₆ alkyl can be substituted 1 or 2 times with a substituent selected from hydroxy and an unsubstituted C₁₋₄ alkoxy, wherein the substituted monocyclic C₃₋₆ cycloalkyl can be substituted 1, 2, 3 or 4 times with a substituent independently selected from halogen, an unsubstituted C₁₋₄ alkyl, an unsubstituted C₁₋₄ alkoxy, an unsubstituted C₁₋₄ haloalkyl and an unsubstituted monocyclic C₃₋₆ cycloalkyl, and wherein the substituted C₁₋₆ haloalkyl can be substituted 1 or 2 times with an unsubstituted C₁₋₄ alkoxy; R¹¹ can be an unsubstituted or a substituted monocyclic 4- to 6-membered heterocyclyl, —(NH)ₘ-(an unsubstituted or a substituted 5- to 6-membered monocyclic heteroaryl), —O-(an unsubstituted or a substituted C₁₋₆ alkyl), —O-(an unsubstituted or a substituted C₃₋₈ cycloalkyl) or —O—(C₁₋₄ alkyl)-(an unsubstituted or a substituted C₃₋₈ cycloalkyl), wherein m can be 0 or 1; R¹² can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl), an unsubstituted or a substituted heteroaryl(alkyl), an unsubstituted or a substituted heterocyclyl(alkyl), an unsubstituted or a substituted C-carboxy, —OR¹³, —NR¹⁴R¹⁵ or —C(═O)—NR^{16A}R^{16B}; R¹³ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl); R¹⁴ are R can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) and an unsubstituted or a substituted heteroaryl(alkyl); R^{16A} can be hydrogen or an unsubstituted C₁₋₃ alkyl; R^{16B} can be an unsubstituted or a substituted aryl, an unsubstituted or a substituted heteroaryl or an unsubstituted or a substituted 3- to 8-membered monocyclic heterocyclyl; and R¹⁷ and R¹⁸ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl); or R¹⁷ and R¹⁸ can be taken together along with the nitrogen to which they are connected to form an unsubstituted or a substituted 3-8 membered heterocyclyl.

In some embodiments, R¹ cannot be

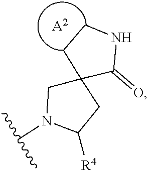

wherein Ring A² is an unsubstituted or a substituted phenyl, an unsubstituted or a substituted monocyclic heteroaryl, an unsubstituted or a substituted monocyclic heterocyclyl, an unsubstituted or a substituted bicyclic heteroaryl or an unsubstituted or a substituted bicyclic heterocyclyl. In some embodiments, R¹ cannot be

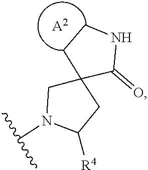

wherein Ring A² is an unsubstituted or a substituted phenyl. In other embodiments, R¹ cannot be

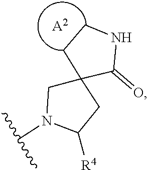

wherein Ring A² is an unsubstituted or a substituted heteroaryl (such as an unsubstituted or a substituted monocyclic heteroaryl and/or an unsubstituted or a substituted bicyclic heteroaryl). In still other embodiments, R¹ cannot be

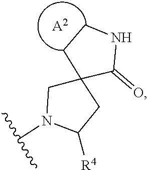

wherein Ring A² is an unsubstituted or a substituted heterocyclyl (for example, an unsubstituted or a substituted monocyclic heterocyclyl and/or an unsubstituted or a substituted bicyclic heterocyclyl). In some embodiments, Ring A² of

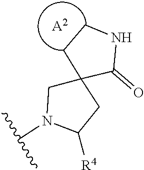

cannot be selected from:

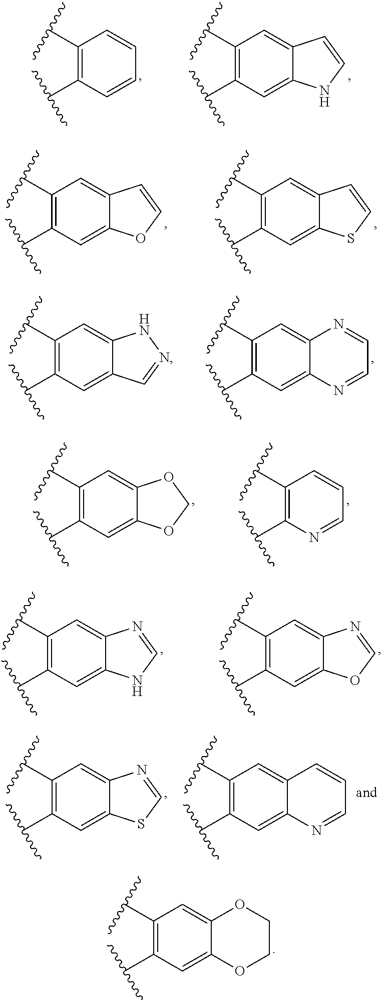

In some embodiments, R⁴ cannot be cyano. In some embodiments, R⁴ cannot be an unsubstituted or a substituted acyl or an unsubstituted or a substituted ketoamide. For example, R⁴ cannot be —C(═O)H, —C(═O)—C(═O)(an unsubstituted cyclopropyl), —C(═O)—C(═O)(an unsubstituted benzyl), —C(═O)—C(═O)(an unsubstituted cyclohexyl). In some embodiments, R⁴ cannot be —C(═O)NH₂. In some embodiments, R⁴ cannot be —CH(OH)—(S(═O)₂—OH) or —CH(OH)—(S(═O)₂—O—). In some embodiments, R^{5a} cannot be hydrogen. In some embodiments, R² cannot be an unsubstituted C₁₋₈ alkyl. In some embodiments, R² cannot be a substituted C₁₋₈ alkyl. In some embodiments, R² cannot be isopropyl or cyclopropyl-CH₂—. In some embodiments, R² cannot be isopropyl, n-butyl, —CH₂—(CH₃)₃, cyclopropyl-CH₂—, cyclobutyl-CH₂—, unsubstituted benzyl or a halogen-substituted benzyl (such as a para-fluoro-substituted benzyl). In some embodiments, a compound of Formula (I), or a pharmaceutically acceptable salt thereof, cannot be a compound, or a pharmaceutically acceptable salt, provided in WO 2022/109363, WO 2022/109360, U.S. Pat. Nos. 11,325,916, 11,339,170, 11,384,090 and 11,352,363.

In some embodiment, R^{N} cannot be a C₁₋₆ alkyl, such as methyl. In some embodiments, R¹² cannot be an unsubstituted or a substituted heteroaryl. For example, in some embodiments, R¹² cannot be an unsubstituted or a substituted indolyl. In some embodiments, R¹² cannot be an unsubstituted, halogen-substituted indolyl or alkoxy-substituted indolyl. In some embodiments, R¹² cannot be an unsubstituted or a substituted,

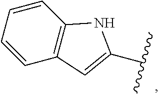 [SMILES: *c1cc2ccccc2[nH]1]

for example,

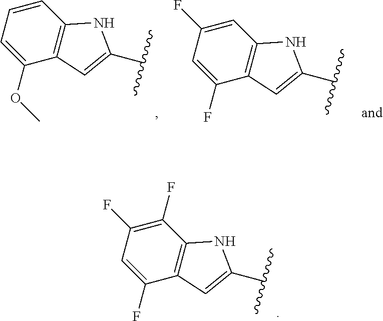 [SMILES: *c1cc2c(OC)cccc2[nH]1 ; *c1cc2c(F)cc(F)cc2[nH]1 ; *c1cc2c(F)cc(F)c(F)c2[nH]1]

In some embodiments, R¹² cannot be an unsubstituted or a substituted heterocyclyl. In some embodiments, R¹² cannot be an unsubstituted or a substituted bicyclic heterocyclyl, such as an unsubstituted or a substituted 5,6,7,8-tetrahydroquinoline, an unsubstituted or a substituted 5,6,7,8-tetrahydroisoquinoline, an unsubstituted or a substituted 5,6,7,8-tetrahydroisoquinolin-1(2H)-one or an unsubstituted or a substituted 2,5,6,7-tetrahydro-1H-cyclopenta[c]pyridin-1-one. In some embodiments, R¹² cannot be selected from

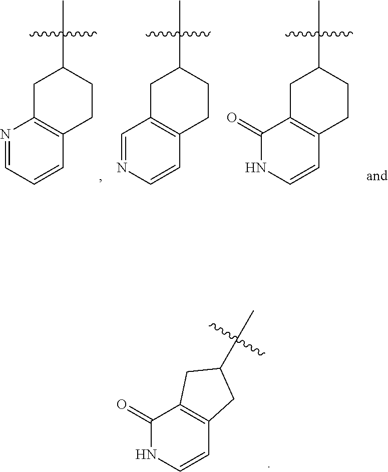

In some embodiments, R¹² cannot be one or more of the following:

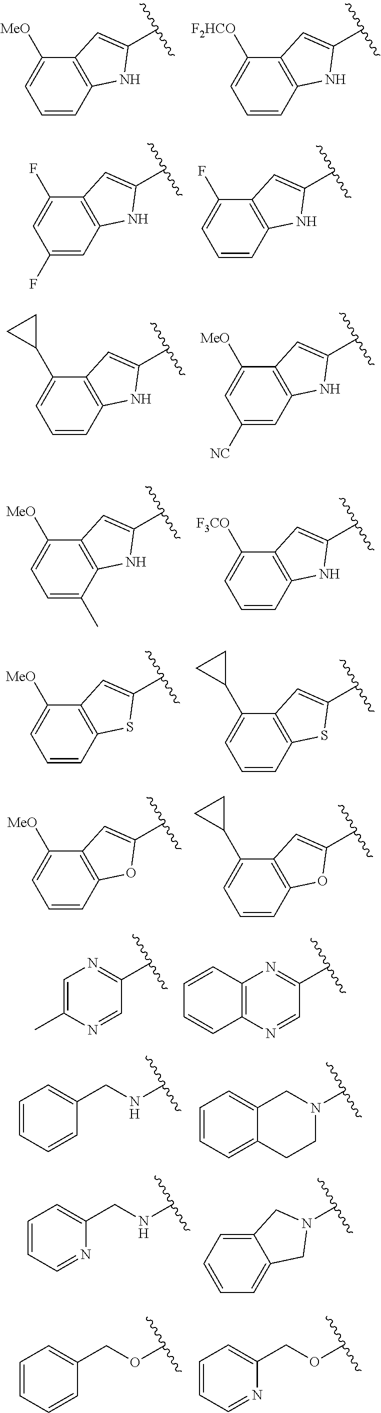

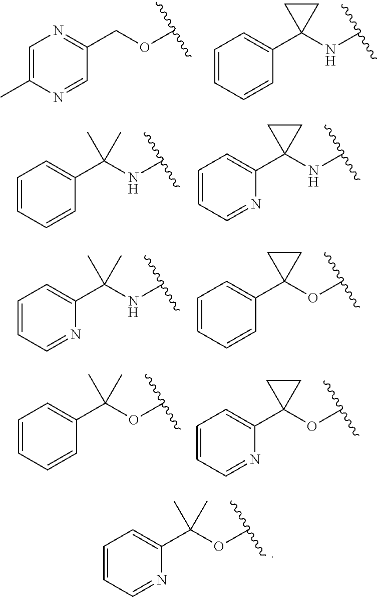

In some embodiments, R¹² cannot be one or more of the following:

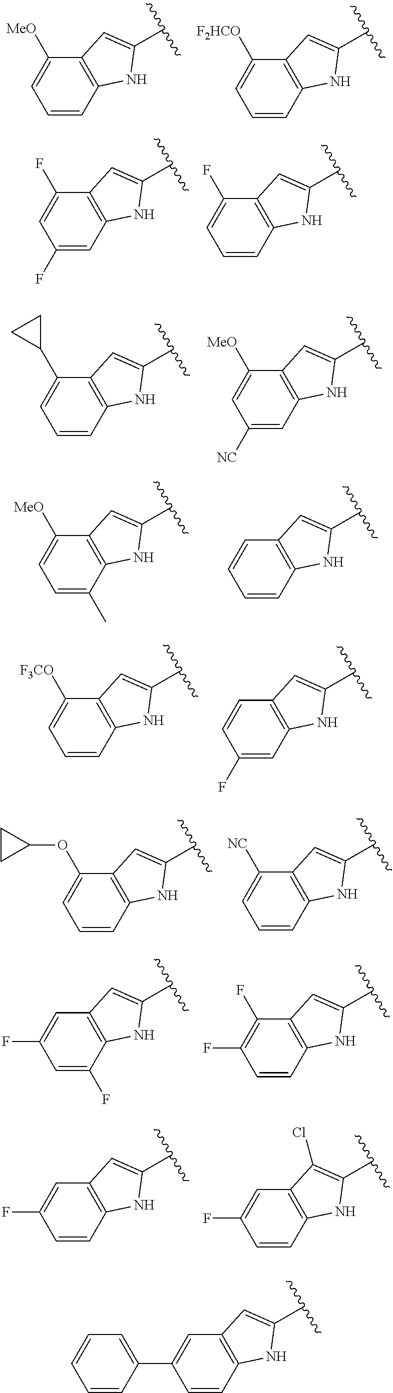

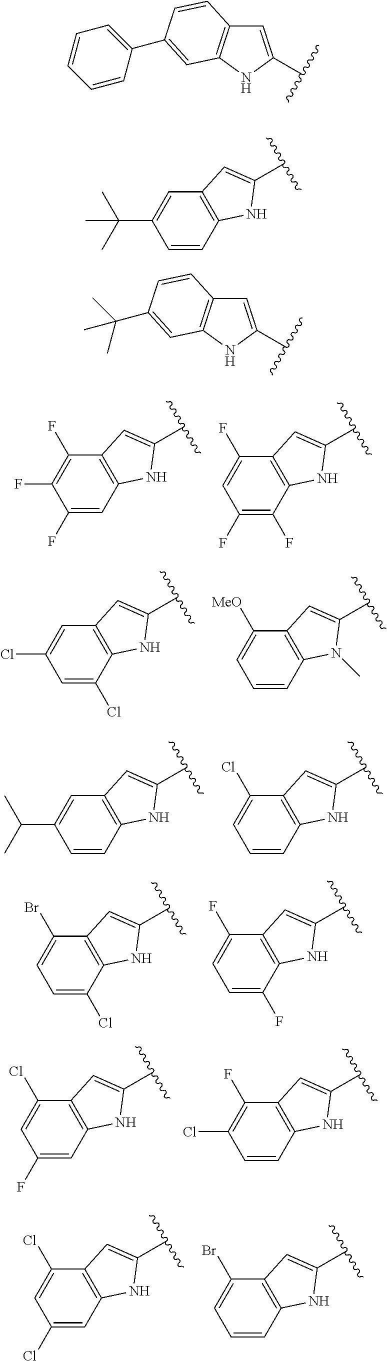

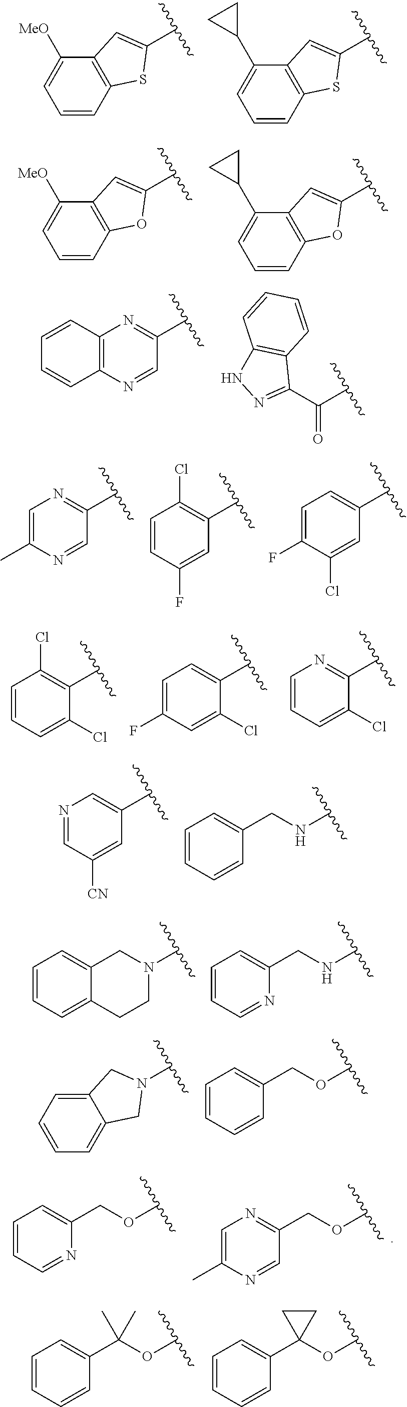

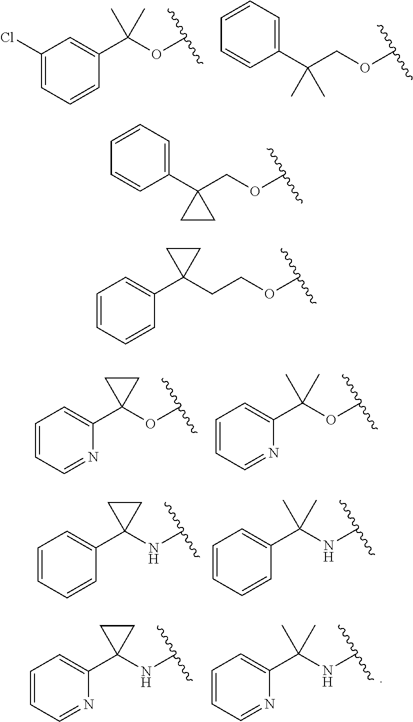

Examples of compounds of Formula (I), include the following:

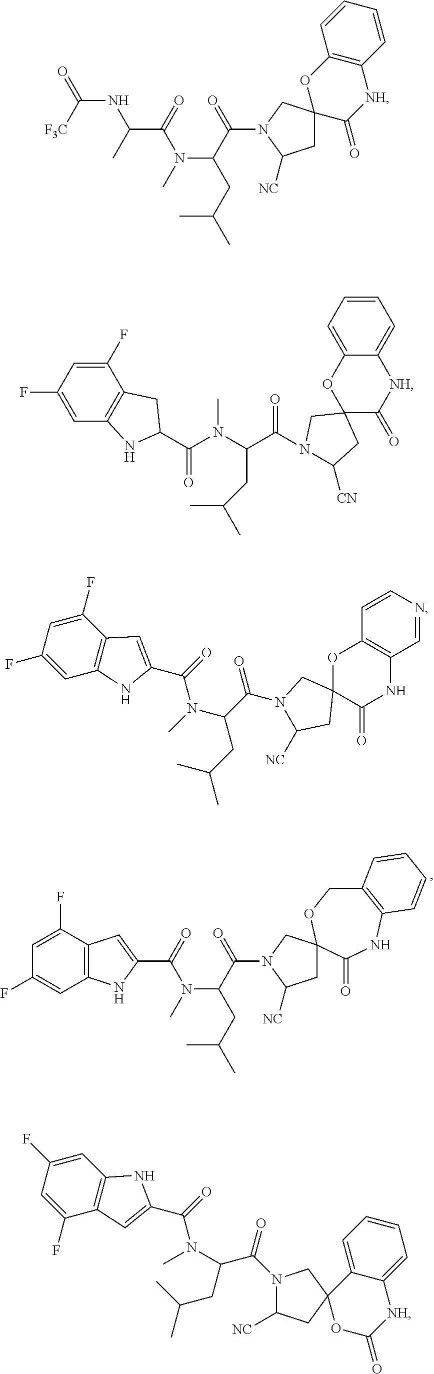 [SMILES: CC(C)CC(C(=O)N1CC2(CC1C#N)Oc1ccccc1NC2=O)N(C)C(=O)C(C)NC(=O)C(F)(F)F ; CC(C)CC(C(=O)N1CC2(CC1C#N)Oc1ccccc1NC2=O)N(C)C(=O)C1Cc2c(F)cc(F)cc2N1 ; CC(C)CC(C(=O)N1CC2(CC1C#N)Oc1ccncc1NC2=O)N(C)C(=O)c1cc2c(F)cc(F)cc2[nH]1 ; CC(C)CC(C(=O)N1CC2(CC1C#N)OCc1ccccc1NC2=O)N(C)C(=O)c1cc2c(F)cc(F)cc2[nH]1 ; "," ; CC(C)CC(C(=O)N1CC2(CC1C#N)OC(=O)Nc1ccccc12)N(C)C(=O)c1cc2c(F)cc(F)cc2[nH]1]

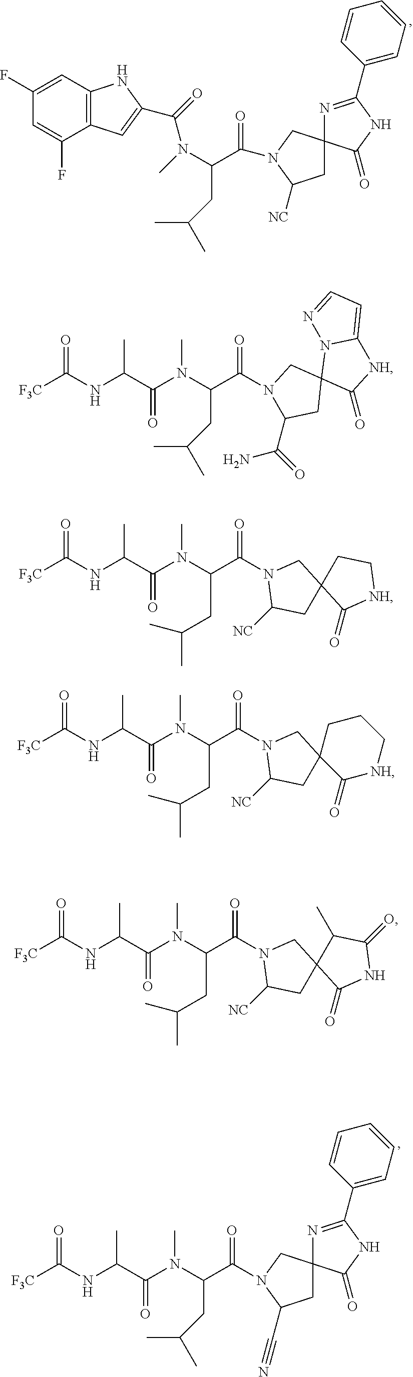

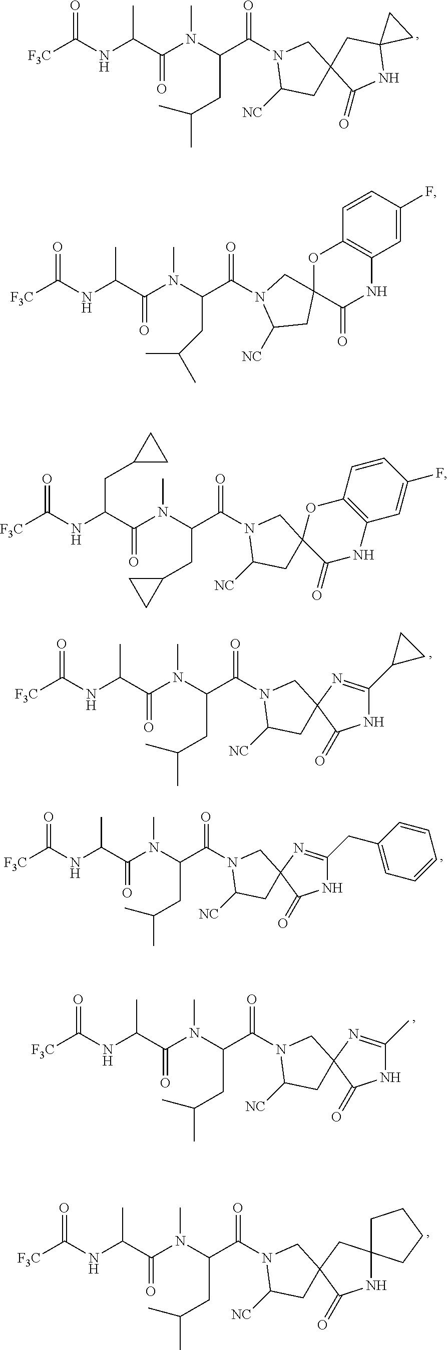

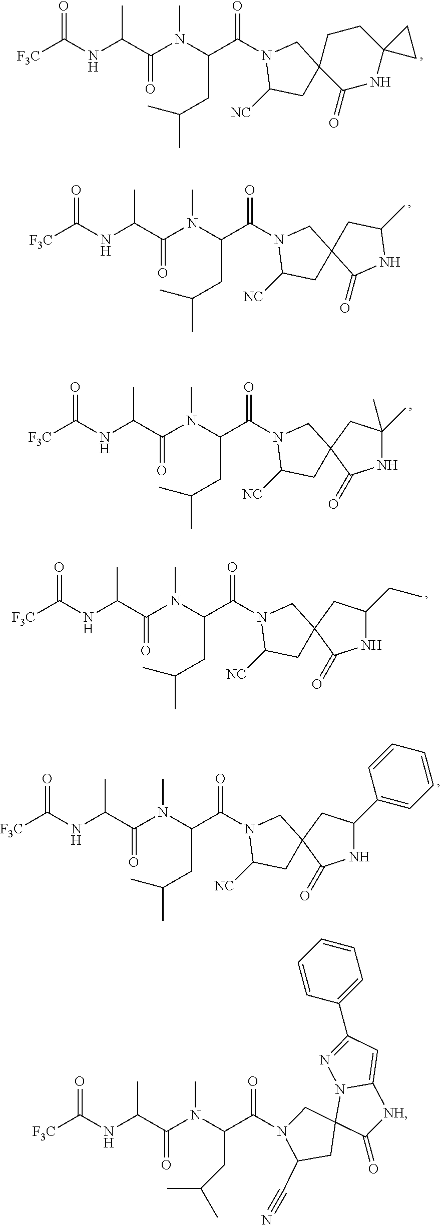

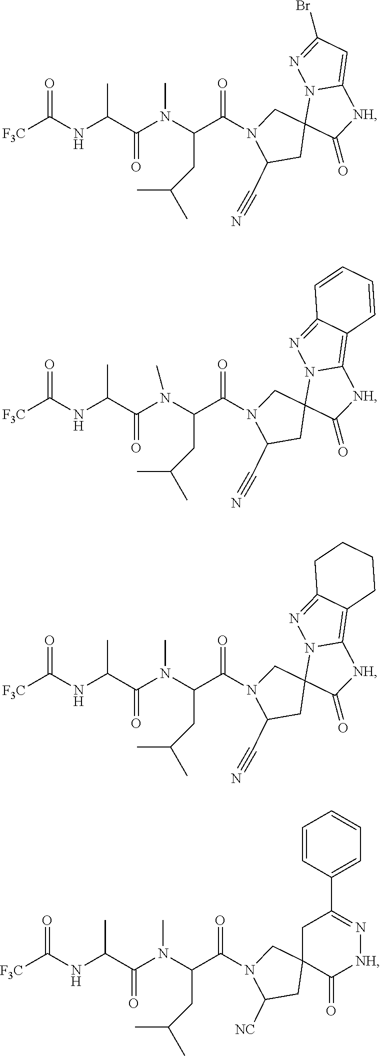 [SMILES: CC(C)CC(C(=O)N1CC2(CC1C#N)C(=O)Nc1cc(Br)nn12)N(C)C(=O)C(C)NC(=O)C(F)(F)F ; CC(C)CC(C(=O)N1CC2(CC1C#N)C(=O)Nc1c3ccccc3nn12)N(C)C(=O)C(C)NC(=O)C(F)(F)F ; CC(C)CC(C(=O)N1CC2(CC1C#N)C(=O)Nc1c3c(nn12)CCCC3)N(C)C(=O)C(C)NC(=O)C(F)(F)F ; CC(C)CC(C(=O)N1CC2(CC(c3ccccc3)=NNC2=O)CC1C#N)N(C)C(=O)C(C)NC(=O)C(F)(F)F]

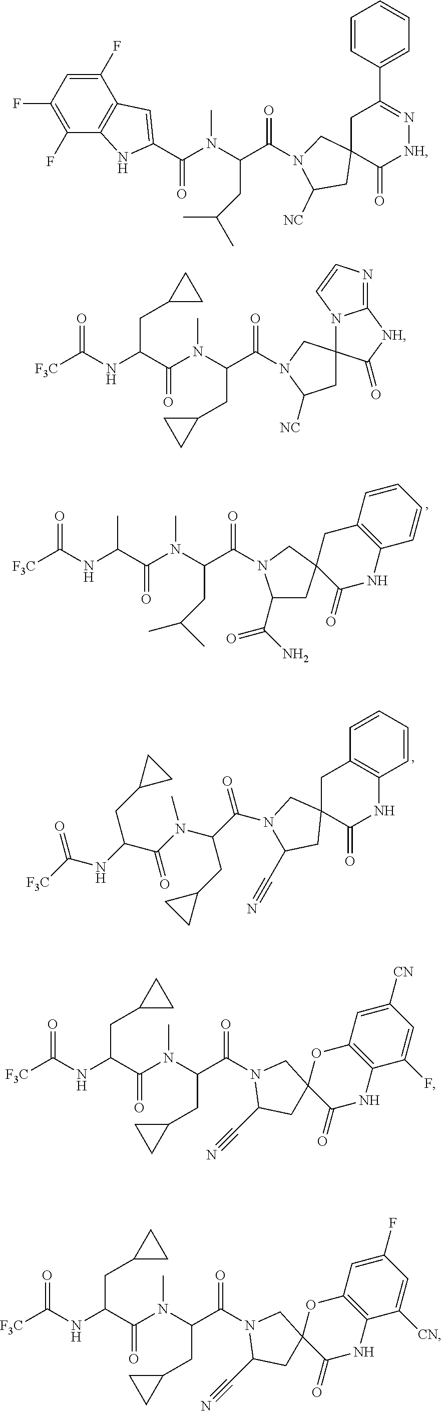

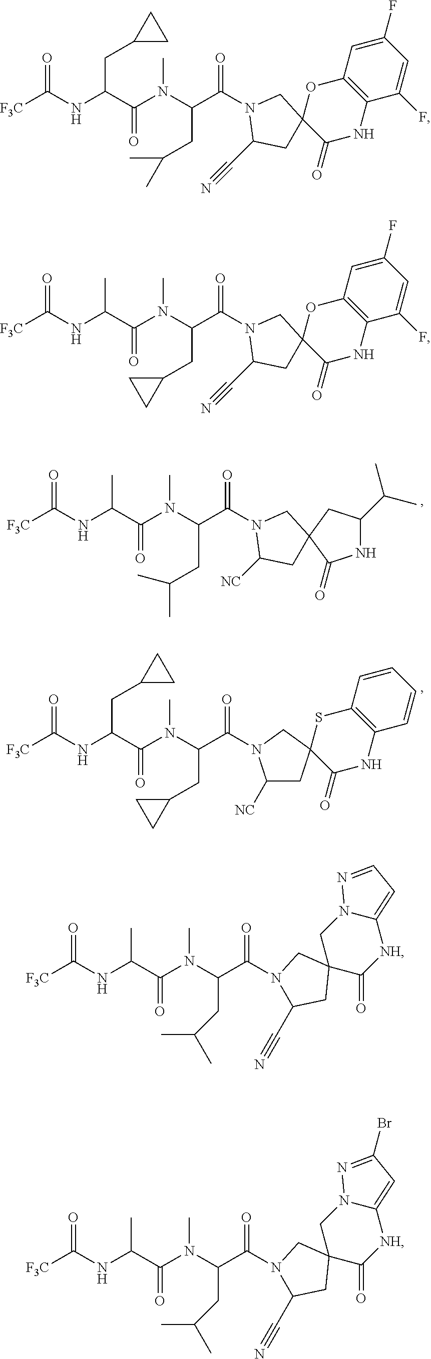

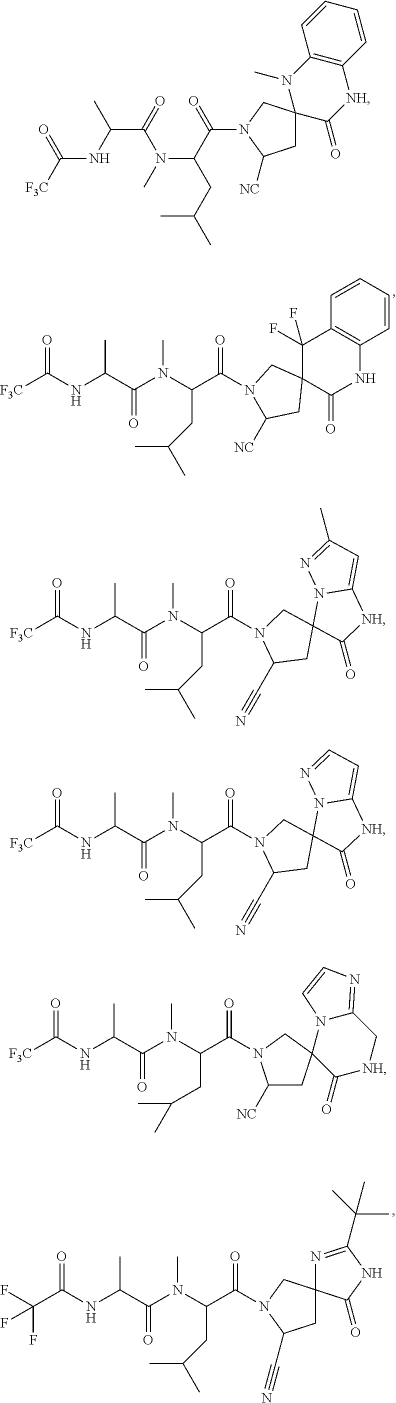

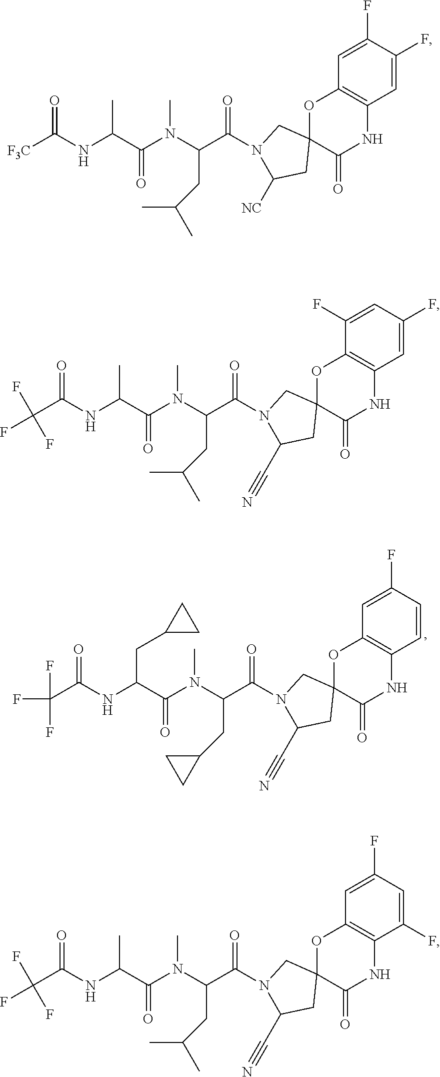 [SMILES: CC(C)CC(C(=O)N1CC2(CC1C#N)Oc1cc(F)c(F)cc1NC2=O)N(C)C(=O)C(C)NC(=O)C(F)(F)F ; CC(C)CC(C(=O)N1CC2(CC1C#N)Oc1c(F)cc(F)cc1NC2=O)N(C)C(=O)C(C)NC(=O)C(F)(F)F ; CN(C(=O)C(CC1CC1)NC(=O)C(F)(F)F)C(CC1CC1)C(=O)N1CC2(CC1C#N)Oc1cc(F)ccc1NC2=O ; "," ; CC(C)CC(C(=O)N1CC2(CC1C#N)Oc1cc(F)cc(F)c1NC2=O)N(C)C(=O)C(C)NC(=O)C(F)(F)F]

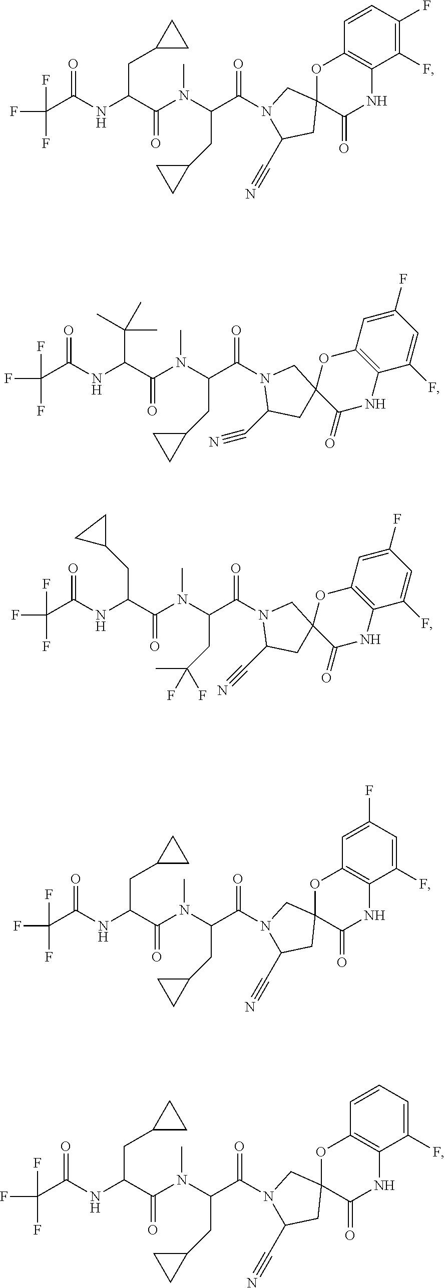 [SMILES: CN(C(=O)C(CC1CC1)NC(=O)C(F)(F)F)C(CC1CC1)C(=O)N1CC2(CC1C#N)Oc1ccc(F)c(F)c1NC2=O ; CN(C(=O)C(NC(=O)C(F)(F)F)C(C)(C)C)C(CC1CC1)C(=O)N1CC2(CC1C#N)Oc1cc(F)cc(F)c1NC2=O ; CN(C(=O)C(CC1CC1)NC(=O)C(F)(F)F)C(CC(C)(F)F)C(=O)N1CC2(CC1C#N)Oc1cc(F)cc(F)c1NC2=O ; CN(C(=O)C(CC1CC1)NC(=O)C(F)(F)F)C(CC1CC1)C(=O)N1CC2(CC1C#N)Oc1cc(F)cc(F)c1NC2=O ; CN(C(=O)C(CC1CC1)NC(=O)C(F)(F)F)C(CC1CC1)C(=O)N1CC2(CC1C#N)Oc1cccc(F)c1NC2=O]

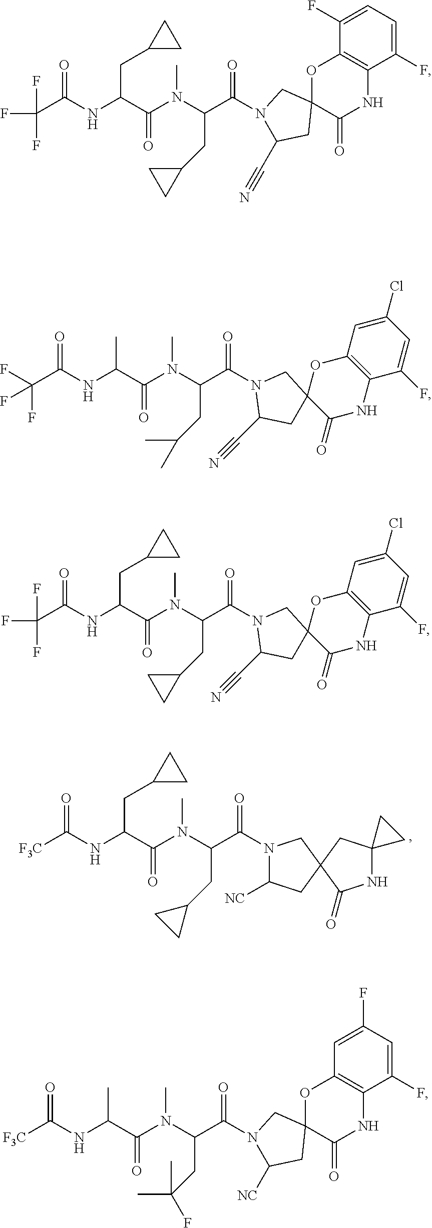 [SMILES: CN(C(=O)C(CC1CC1)NC(=O)C(F)(F)F)C(CC1CC1)C(=O)N1CC2(CC1C#N)Oc1c(F)ccc(F)c1NC2=O ; CC(C)CC(C(=O)N1CC2(CC1C#N)Oc1cc(Cl)cc(F)c1NC2=O)N(C)C(=O)C(C)NC(=O)C(F)(F)F ; CN(C(=O)C(CC1CC1)NC(=O)C(F)(F)F)C(CC1CC1)C(=O)N1CC2(CC1C#N)Oc1cc(Cl)cc(F)c1NC2=O ; CN(C(=O)C(CC1CC1)NC(=O)C(F)(F)F)C(CC1CC1)C(=O)N1CC2(CC1C#N)CC1(CC1)NC2=O ; "," ; CC(NC(=O)C(F)(F)F)C(=O)N(C)C(CC(C)(C)F)C(=O)N1CC2(CC1C#N)Oc1cc(F)cc(F)c1NC2=O]

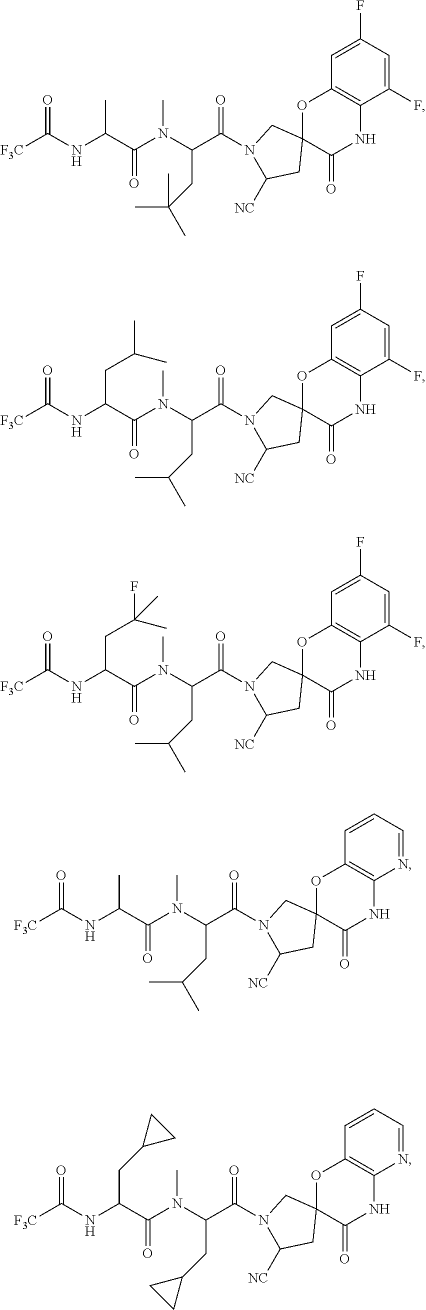 [SMILES: CC(NC(=O)C(F)(F)F)C(=O)N(C)C(CC(C)(C)C)C(=O)N1CC2(CC1C#N)Oc1cc(F)cc(F)c1NC2=O ; CC(C)CC(NC(=O)C(F)(F)F)C(=O)N(C)C(CC(C)C)C(=O)N1CC2(CC1C#N)Oc1cc(F)cc(F)c1NC2=O ; CC(C)CC(C(=O)N1CC2(CC1C#N)Oc1cc(F)cc(F)c1NC2=O)N(C)C(=O)C(CC(C)(C)F)NC(=O)C(F)(F)F ; CC(C)CC(C(=O)N1CC2(CC1C#N)Oc1cccnc1NC2=O)N(C)C(=O)C(C)NC(=O)C(F)(F)F ; CN(C(=O)C(CC1CC1)NC(=O)C(F)(F)F)C(CC1CC1)C(=O)N1CC2(CC1C#N)Oc1cccnc1NC2=O]

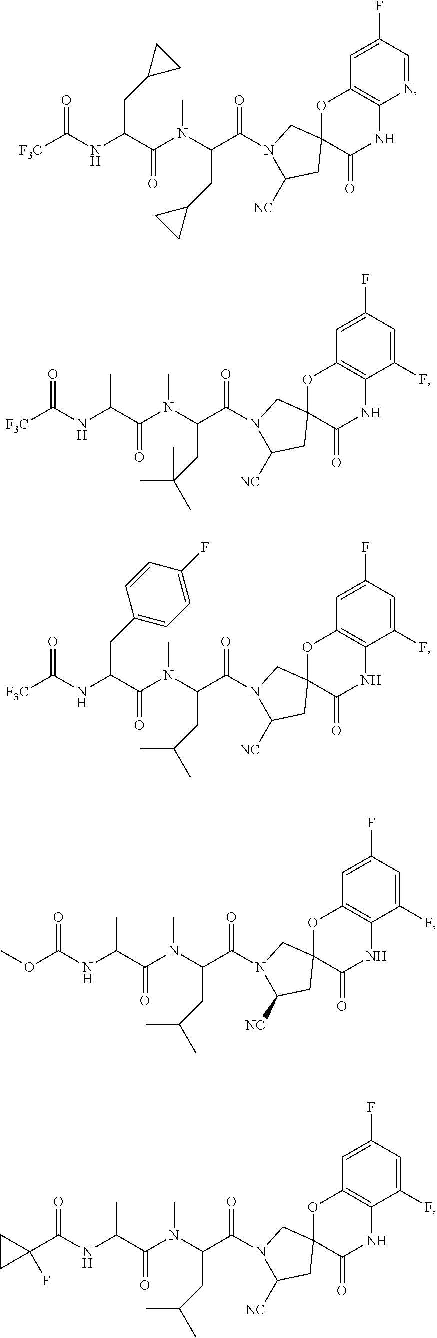

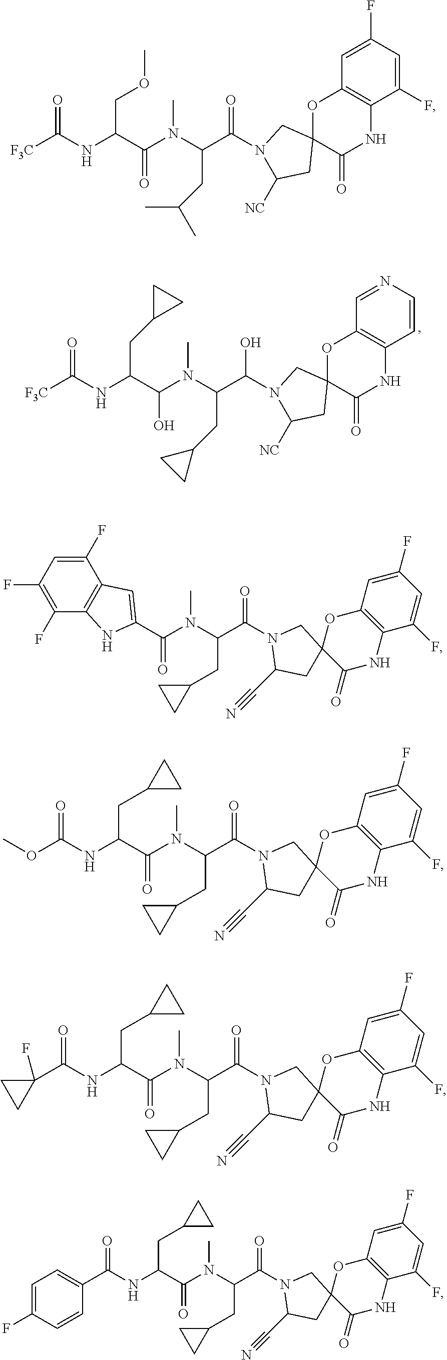

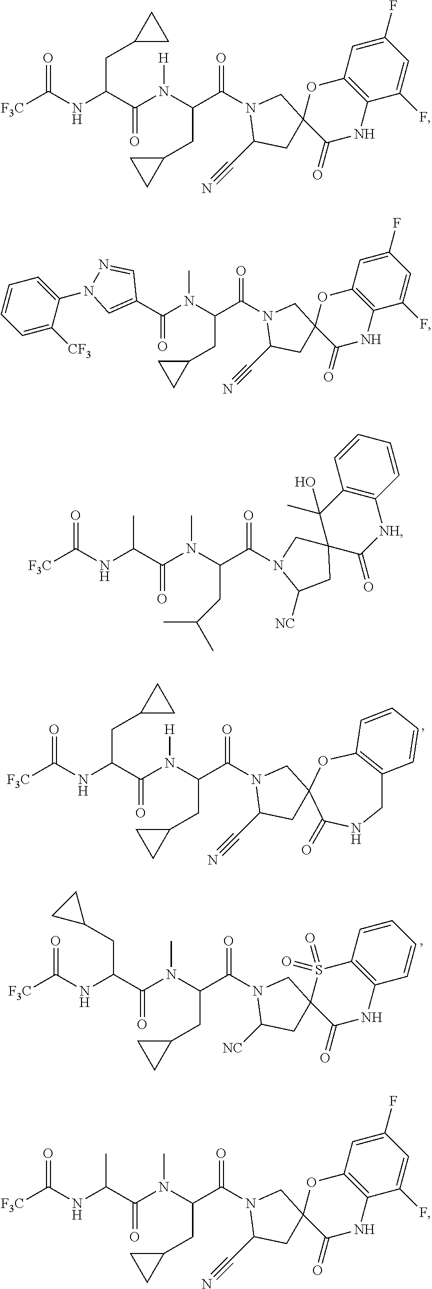

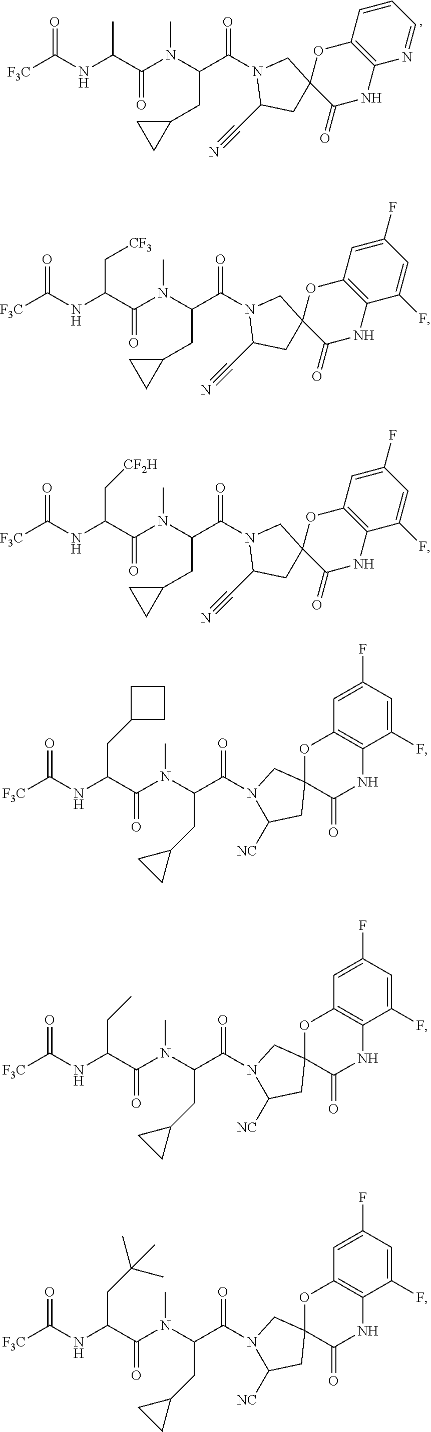

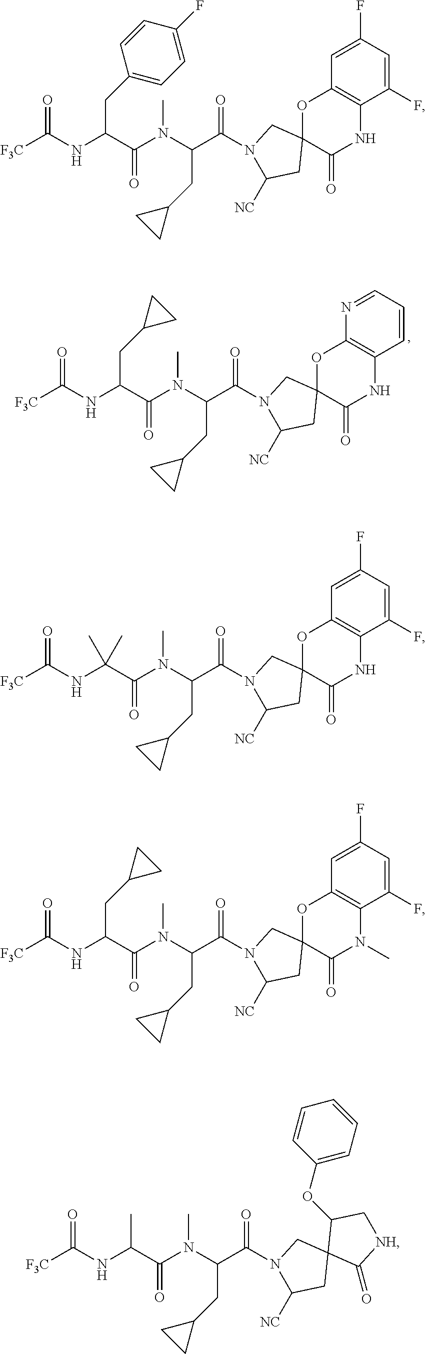 [SMILES: CN(C(=O)C(Cc1ccc(F)cc1)NC(=O)C(F)(F)F)C(CC1CC1)C(=O)N1CC2(CC1C#N)Oc1cc(F)cc(F)c1NC2=O ; CN(C(=O)C(CC1CC1)NC(=O)C(F)(F)F)C(CC1CC1)C(=O)N1CC2(CC1C#N)Oc1ncccc1NC2=O ; "," ; CN(C(=O)C(C)(C)NC(=O)C(F)(F)F)C(CC1CC1)C(=O)N1CC2(CC1C#N)Oc1cc(F)cc(F)c1NC2=O ; CN1C(=O)C2(CC(C#N)N(C(=O)C(CC3CC3)N(C)C(=O)C(CC3CC3)NC(=O)C(F)(F)F)C2)Oc2cc(F)cc(F)c21 ; CC(NC(=O)C(F)(F)F)C(=O)N(C)C(CC1CC1)C(=O)N1CC2(CC1C#N)C(=O)NCC2Oc1ccccc1]

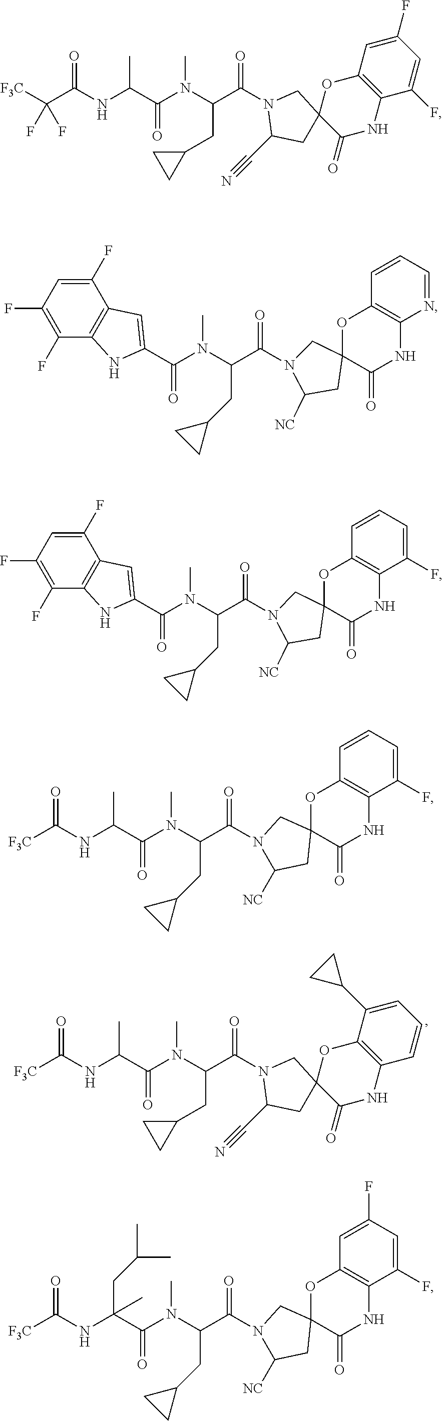

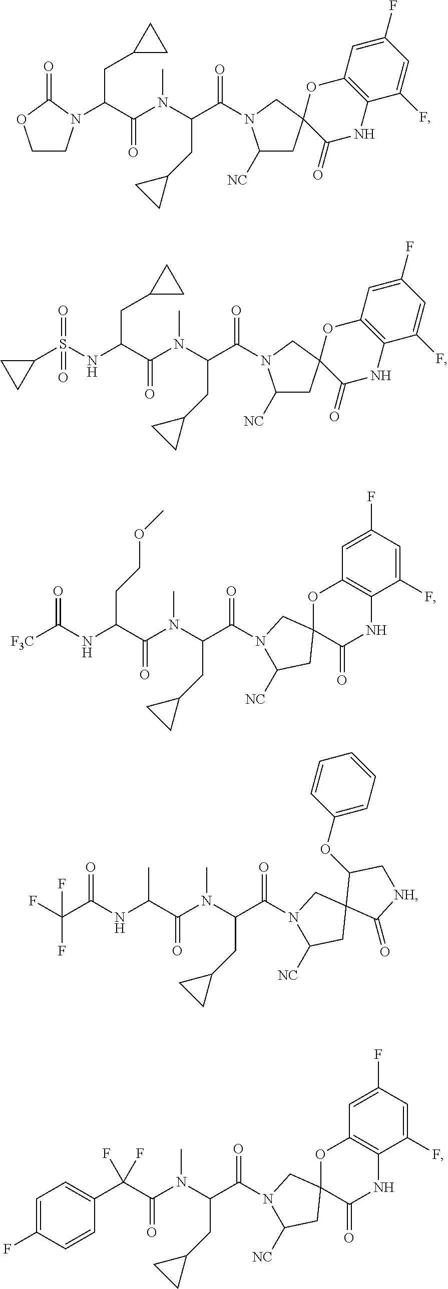 [SMILES: CN(C(=O)C(CC1CC1)N1CCOC1=O)C(CC1CC1)C(=O)N1CC2(CC1C#N)Oc1cc(F)cc(F)c1NC2=O ; CN(C(=O)C(CC1CC1)NS(=O)(=O)C1CC1)C(CC1CC1)C(=O)N1CC2(CC1C#N)Oc1cc(F)cc(F)c1NC2=O ; COCCC(NC(=O)C(F)(F)F)C(=O)N(C)C(CC1CC1)C(=O)N1CC2(CC1C#N)Oc1cc(F)cc(F)c1NC2=O ; CC(NC(=O)C(F)(F)F)C(=O)N(C)C(CC1CC1)C(=O)N1CC2(CC1C#N)C(=O)NCC2Oc1ccccc1 ; CN(C(=O)C(F)(F)c1ccc(F)cc1)C(CC1CC1)C(=O)N1CC2(CC1C#N)Oc1cc(F)cc(F)c1NC2=O]

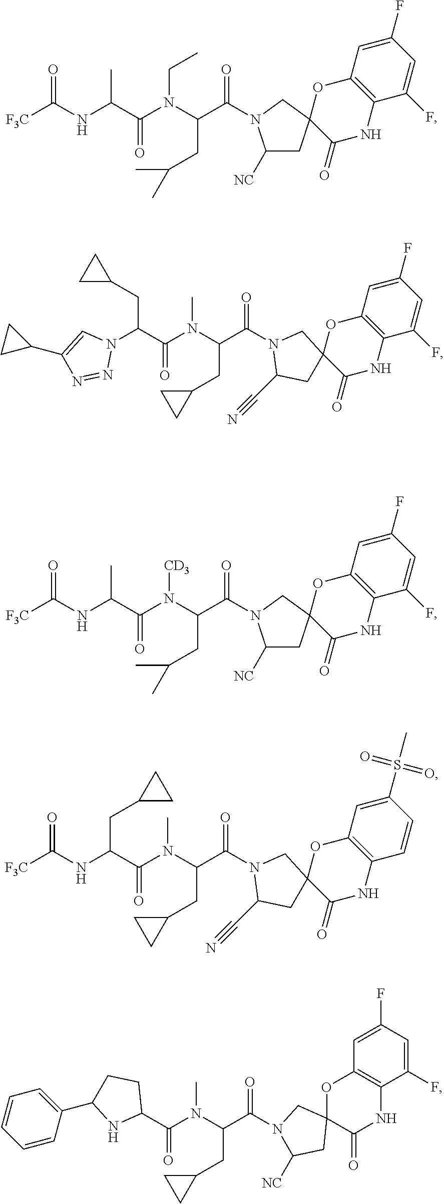 [SMILES: CCN(C(=O)C(C)NC(=O)C(F)(F)F)C(CC(C)C)C(=O)N1CC2(CC1C#N)Oc1cc(F)cc(F)c1NC2=O ; CN(C(=O)C(CC1CC1)n1cc(C2CC2)nn1)C(CC1CC1)C(=O)N1CC2(CC1C#N)Oc1cc(F)cc(F)c1NC2=O ; [2H]C([2H])([2H])N(C(=O)C(C)NC(=O)C(F)(F)F)C(CC(C)C)C(=O)N1CC2(CC1C#N)Oc1cc(F)cc(F)c1NC2=O ; CN(C(=O)C(CC1CC1)NC(=O)C(F)(F)F)C(CC1CC1)C(=O)N1CC2(CC1C#N)Oc1cc(S(C)(=O)=O)ccc1NC2=O ; CN(C(=O)C1CCC(c2ccccc2)N1)C(CC1CC1)C(=O)N1CC2(CC1C#N)Oc1cc(F)cc(F)c1NC2=O]

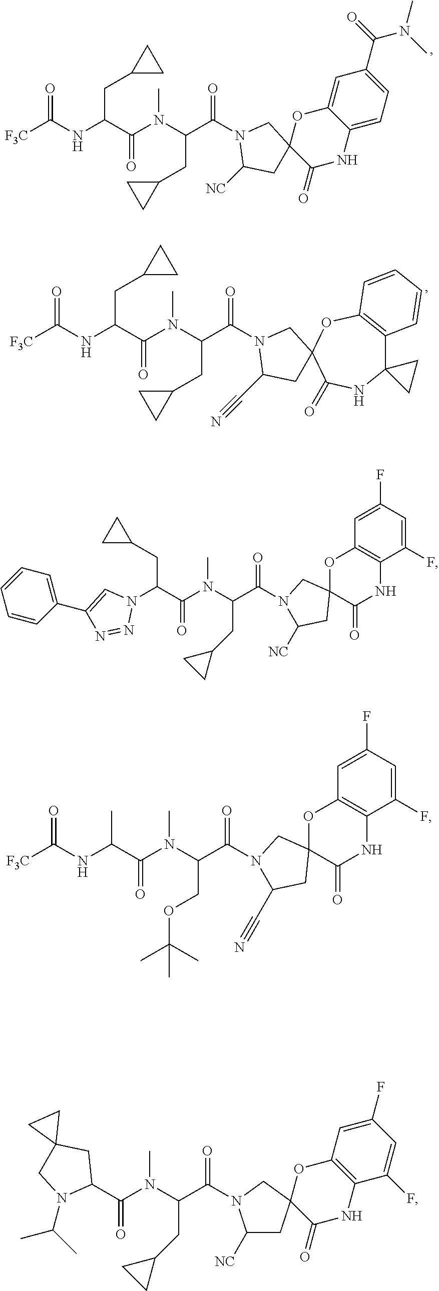

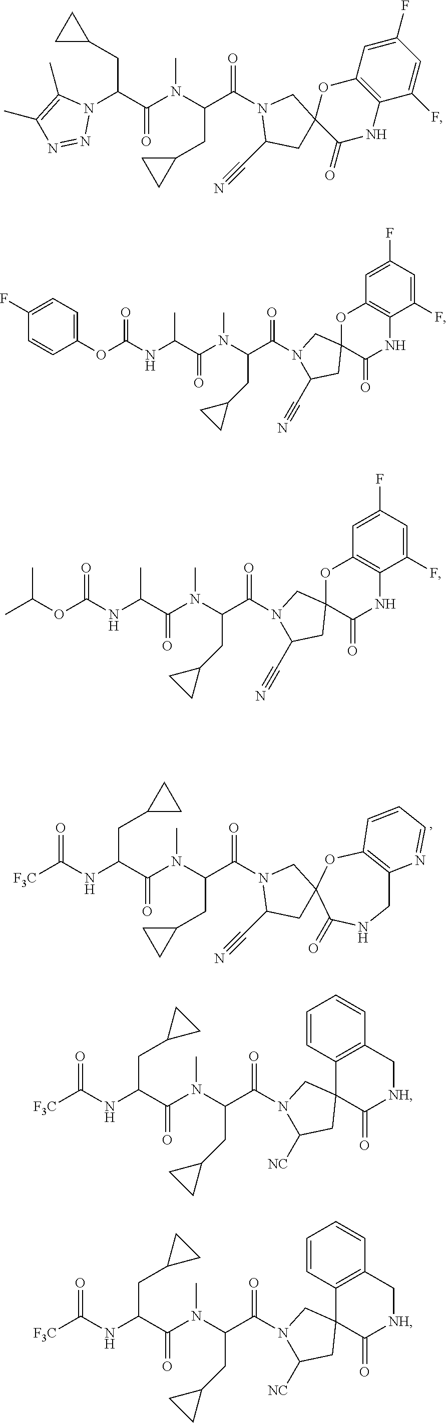

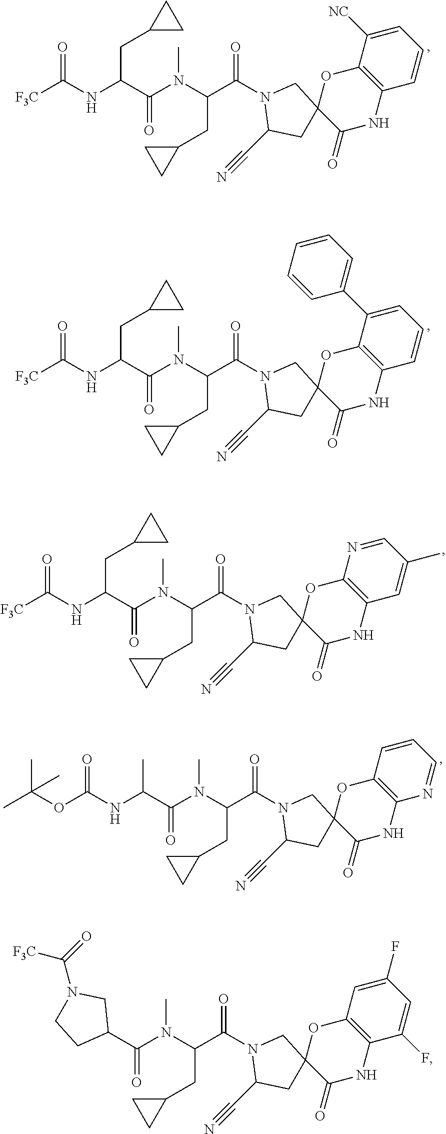

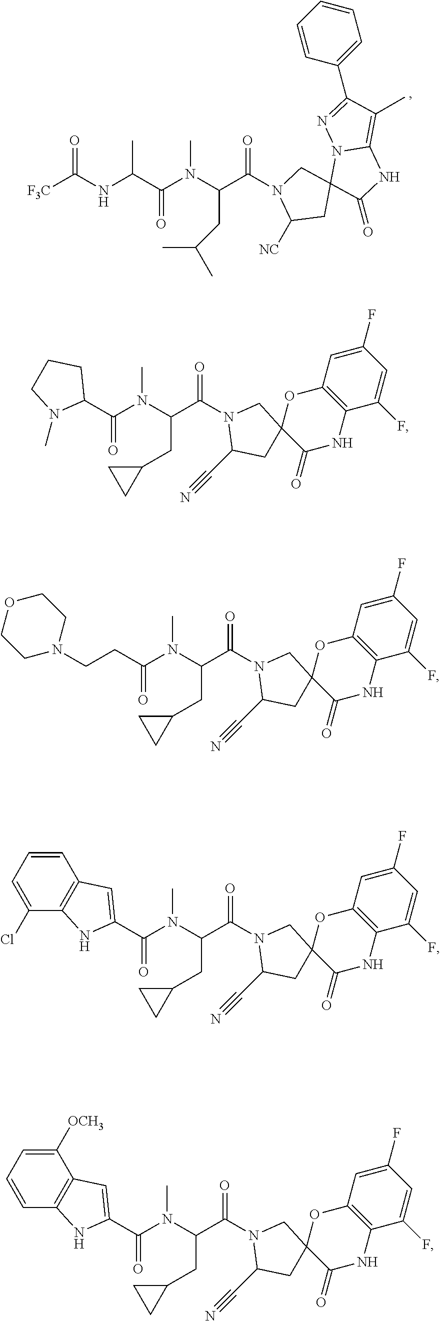 [SMILES: Cc1c(-c2ccccc2)nn2c1NC(=O)C21CC(C#N)N(C(=O)C(CC(C)C)N(C)C(=O)C(C)NC(=O)C(F)(F)F)C1 ; "," ; CN1CCCC1C(=O)N(C)C(CC1CC1)C(=O)N1CC2(CC1C#N)Oc1cc(F)cc(F)c1NC2=O ; CN(C(=O)CCN1CCOCC1)C(CC1CC1)C(=O)N1CC2(CC1C#N)Oc1cc(F)cc(F)c1NC2=O ; CN(C(=O)c1cc2cccc(Cl)c2[nH]1)C(CC1CC1)C(=O)N1CC2(CC1C#N)Oc1cc(F)cc(F)c1NC2=O ; COc1cccc2[nH]c(C(=O)N(C)C(CC3CC3)C(=O)N3CC4(CC3C#N)Oc3cc(F)cc(F)c3NC4=O)cc12]

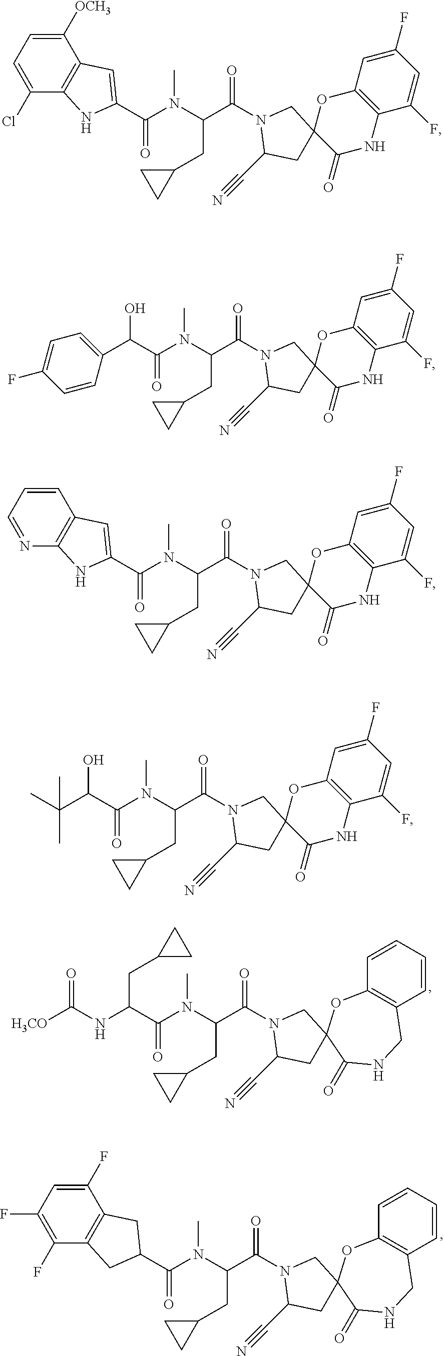

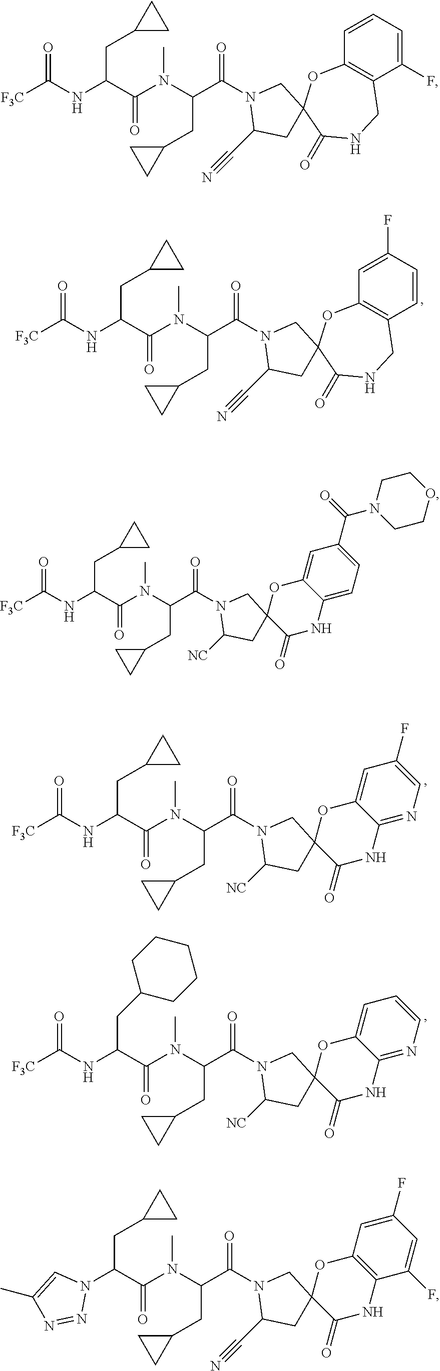

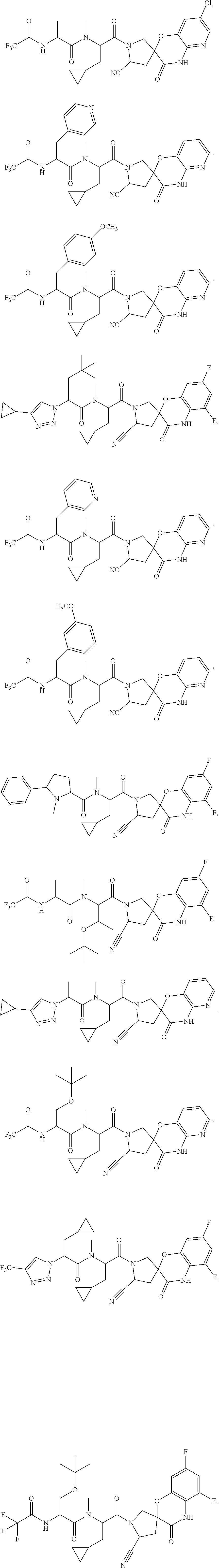

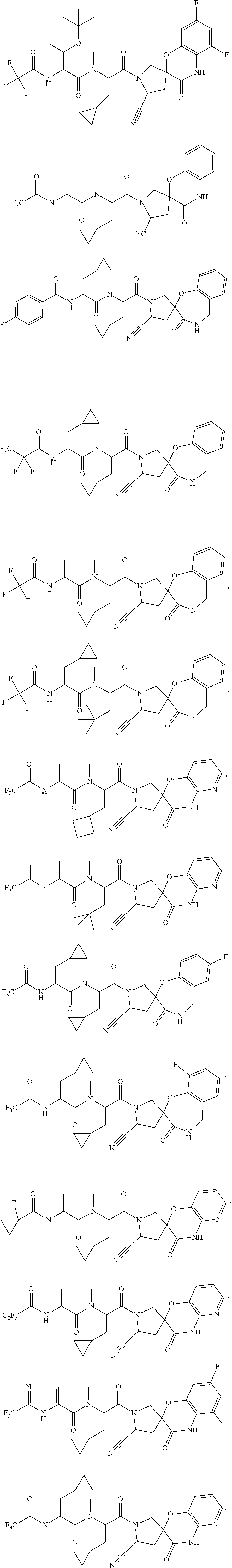

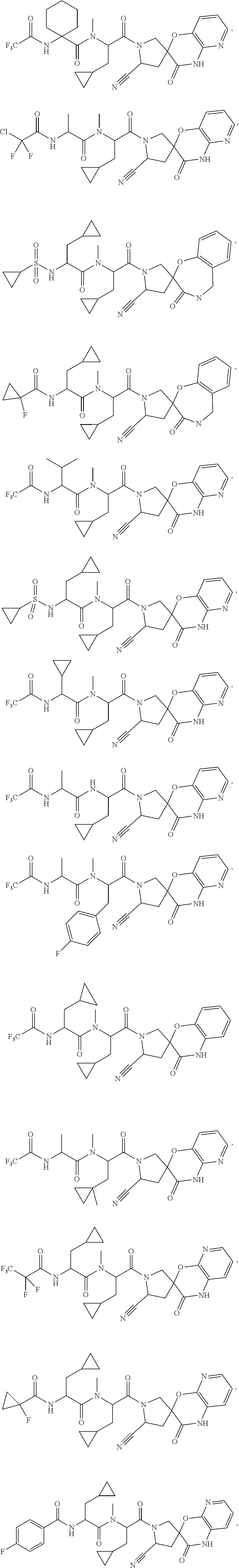

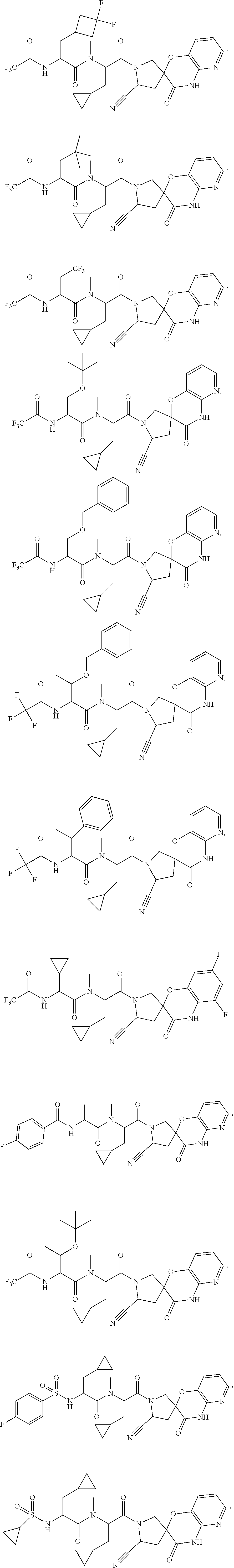

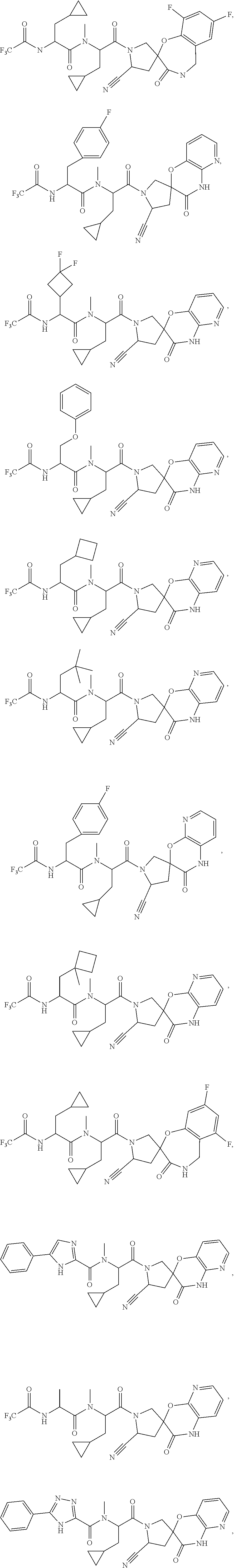

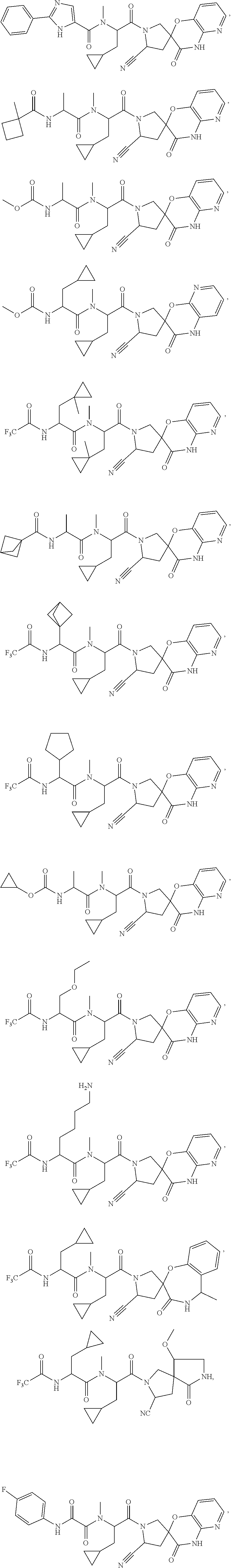

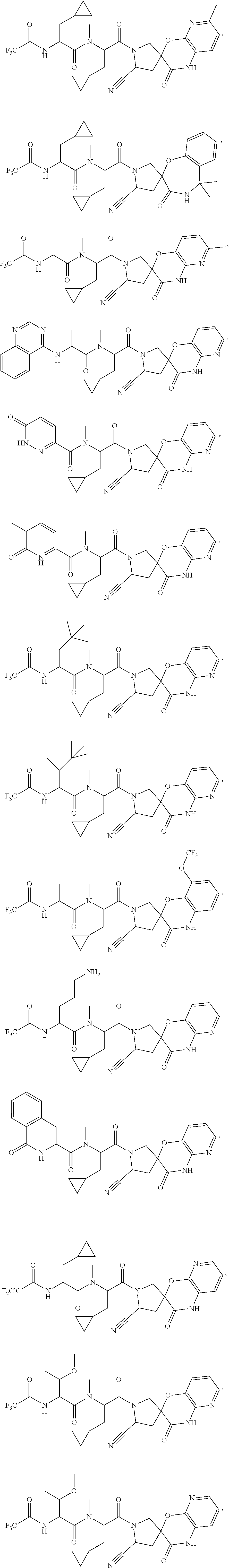

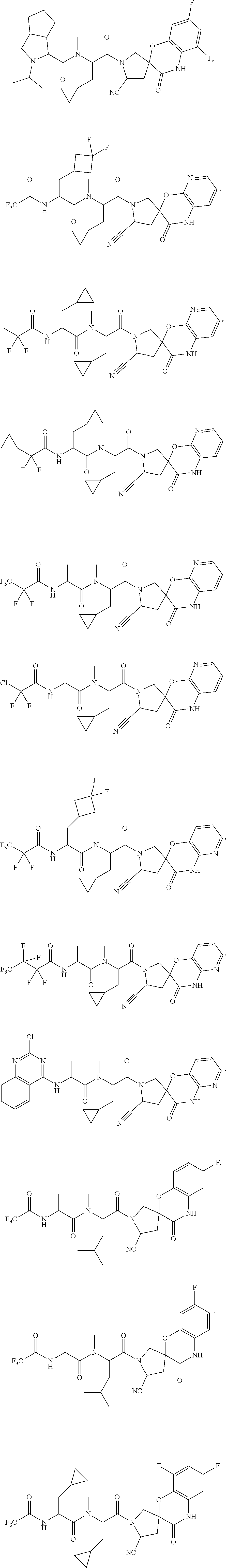

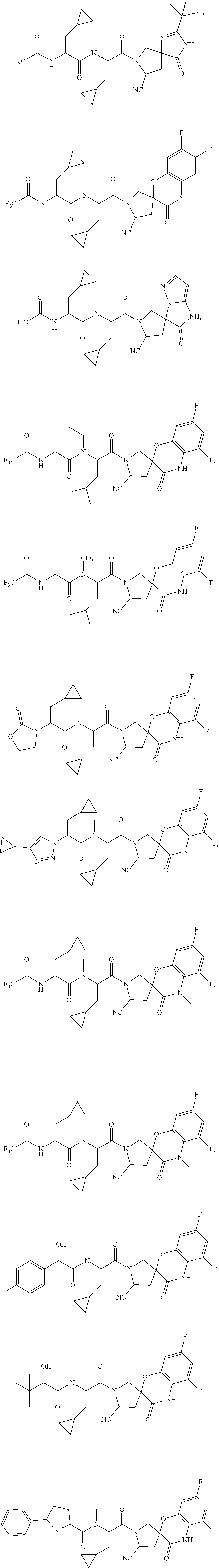

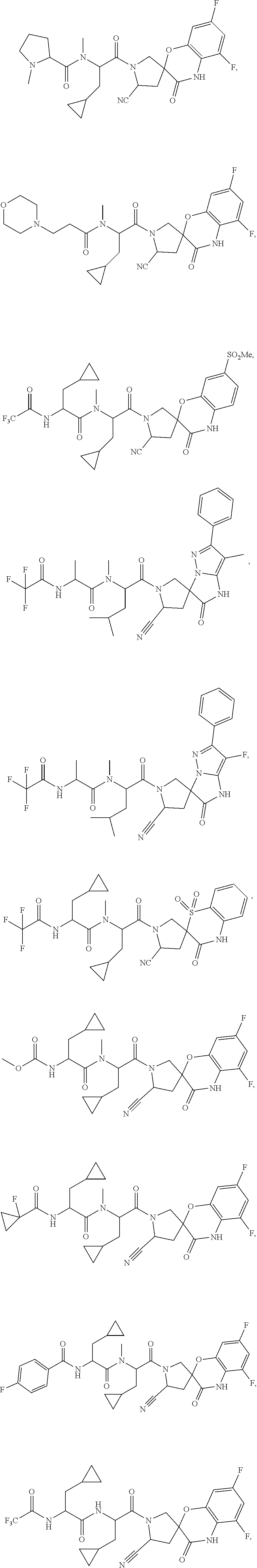

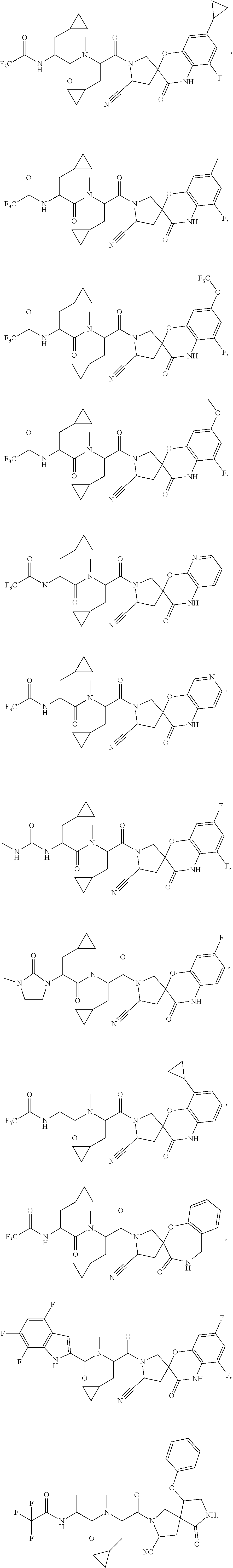

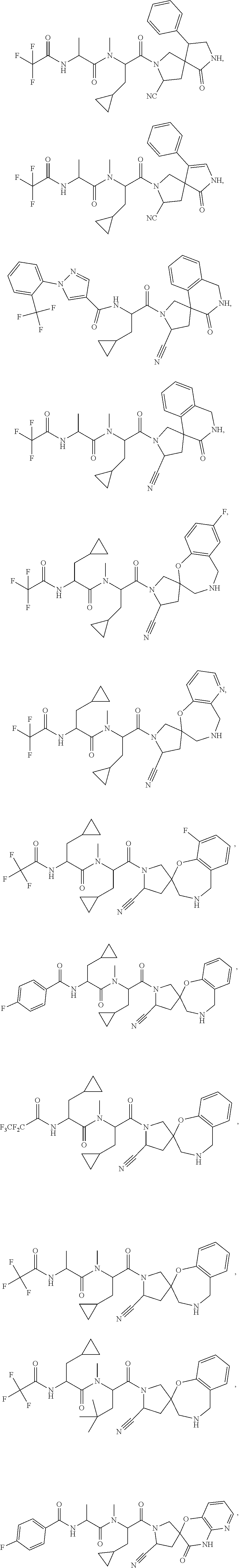

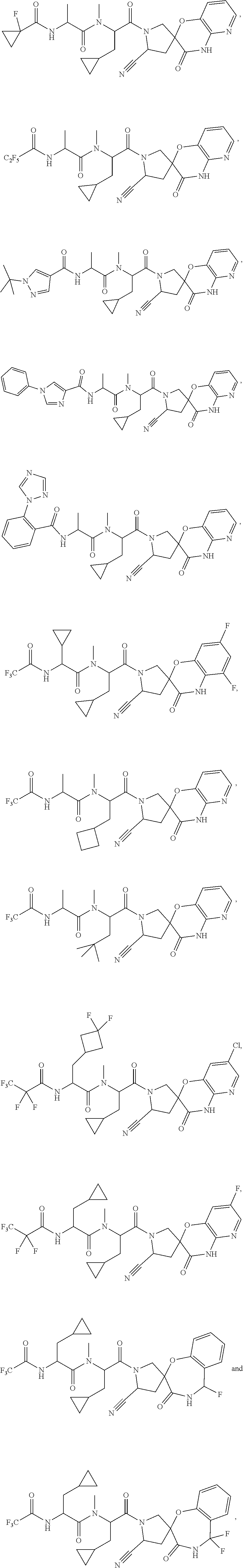

or a pharmaceutically acceptable salt of any of the foregoing.

Additional examples of compounds of Formula (I), include the following:

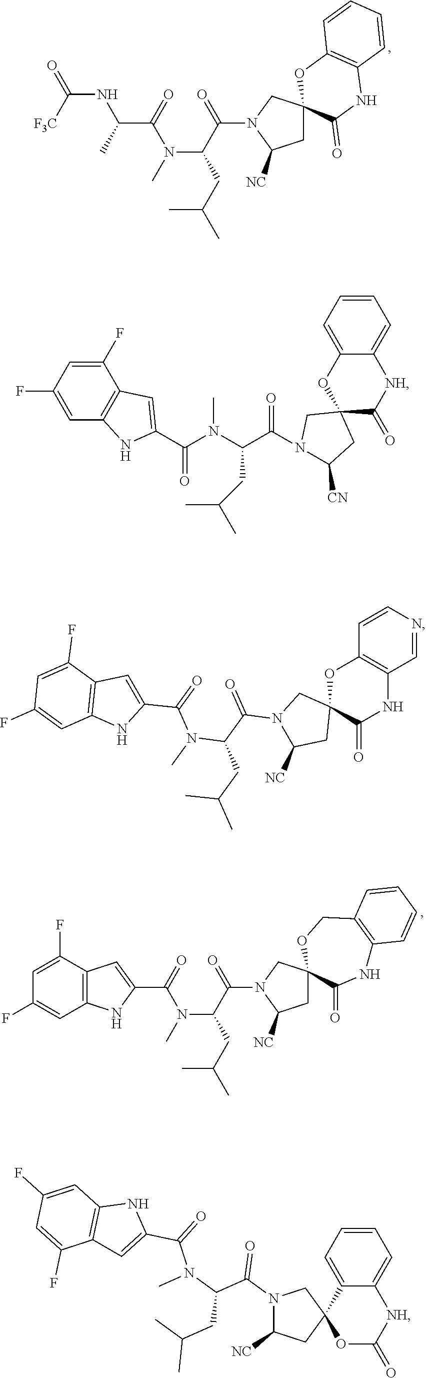 [SMILES: CC(C)C[C@@H](C(=O)N1C[C@@]2(C[C@H]1C#N)Oc1ccccc1NC2=O)N(C)C(=O)[C@H](C)NC(=O)C(F)(F)F ; CC(C)C[C@@H](C(=O)N1C[C@@]2(C[C@H]1C#N)Oc1ccccc1NC2=O)N(C)C(=O)c1cc2c(F)cc(F)cc2[nH]1 ; CC(C)C[C@@H](C(=O)N1C[C@@]2(C[C@H]1C#N)Oc1ccncc1NC2=O)N(C)C(=O)c1cc2c(F)cc(F)cc2[nH]1 ; CC(C)C[C@@H](C(=O)N1C[C@@]2(C[C@H]1C#N)OCc1ccccc1NC2=O)N(C)C(=O)c1cc2c(F)cc(F)cc2[nH]1 ; CC(C)C[C@@H](C(=O)N1C[C@]2(C[C@H]1C#N)OC(=O)Nc1ccccc12)N(C)C(=O)c1cc2c(F)cc(F)cc2[nH]1]

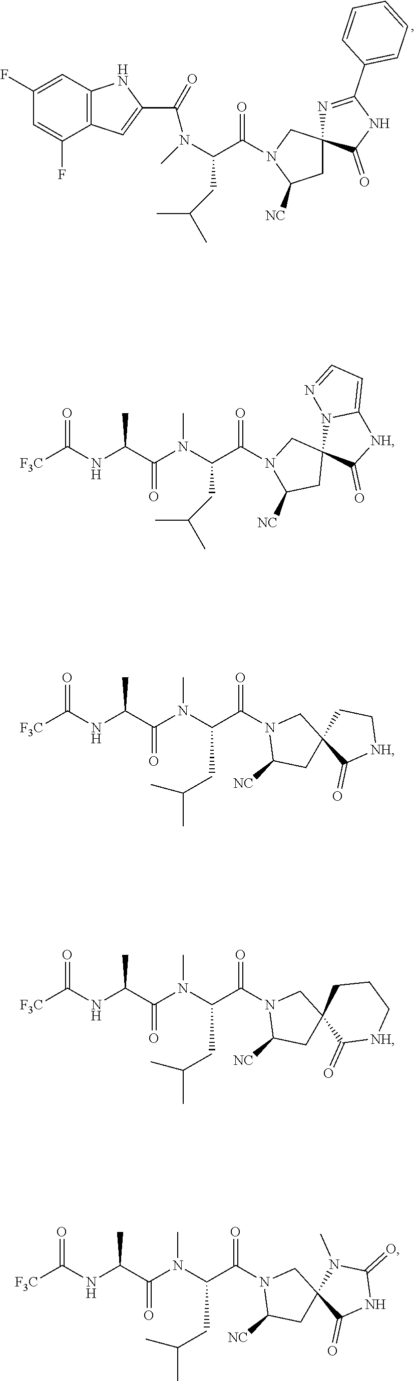 [SMILES: CC(C)C[C@@H](C(=O)N1C[C@@]2(C[C@H]1C#N)N=C(c1ccccc1)NC2=O)N(C)C(=O)c1cc2c(F)cc(F)cc2[nH]1 ; CC(C)C[C@@H](C(=O)N1C[C@]2(C[C@H]1C#N)C(=O)Nc1ccnn12)N(C)C(=O)[C@H](C)NC(=O)C(F)(F)F ; CC(C)C[C@@H](C(=O)N1C[C@]2(CCNC2=O)C[C@H]1C#N)N(C)C(=O)[C@H](C)NC(=O)C(F)(F)F ; CC(C)C[C@@H](C(=O)N1C[C@@]2(CCCNC2=O)C[C@H]1C#N)N(C)C(=O)[C@H](C)NC(=O)C(F)(F)F ; CC(C)C[C@@H](C(=O)N1C[C@]2(C[C@H]1C#N)C(=O)NC(=O)N2C)N(C)C(=O)[C@H](C)NC(=O)C(F)(F)F]

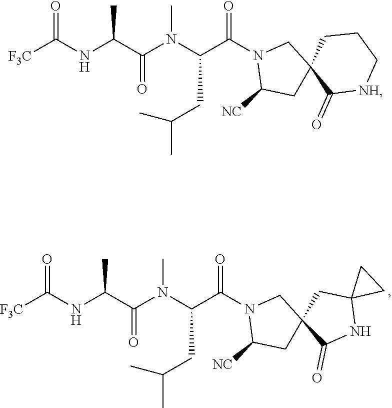 [SMILES: CC(C)C[C@@H](C(=O)N1C[C@]2(CCCNC2=O)C[C@H]1C#N)N(C)C(=O)[C@H](C)NC(=O)C(F)(F)F ; CC(C)C[C@@H](C(=O)N1C[C@@]2(C[C@H]1C#N)CC1(CC1)NC2=O)N(C)C(=O)[C@H](C)NC(=O)C(F)(F)F]

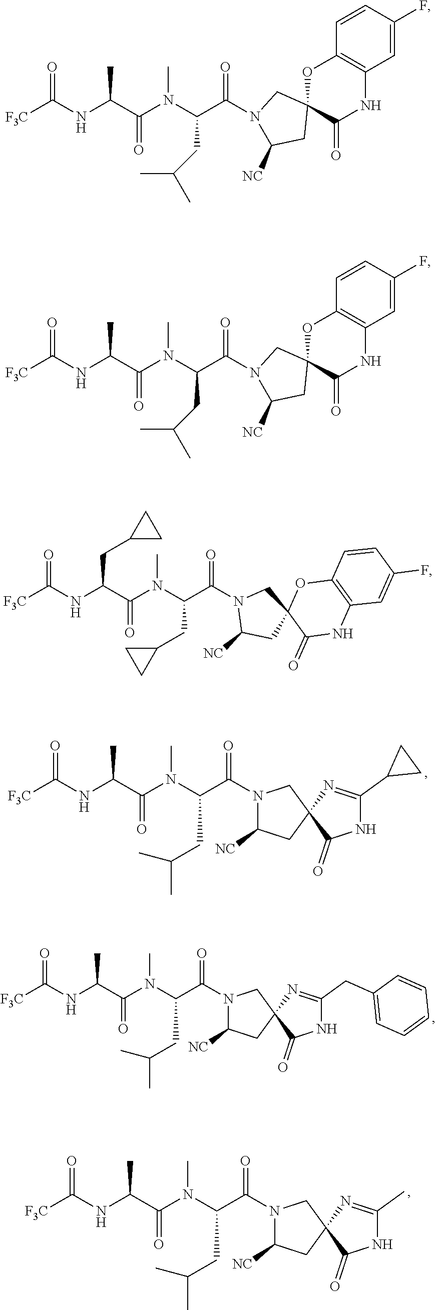

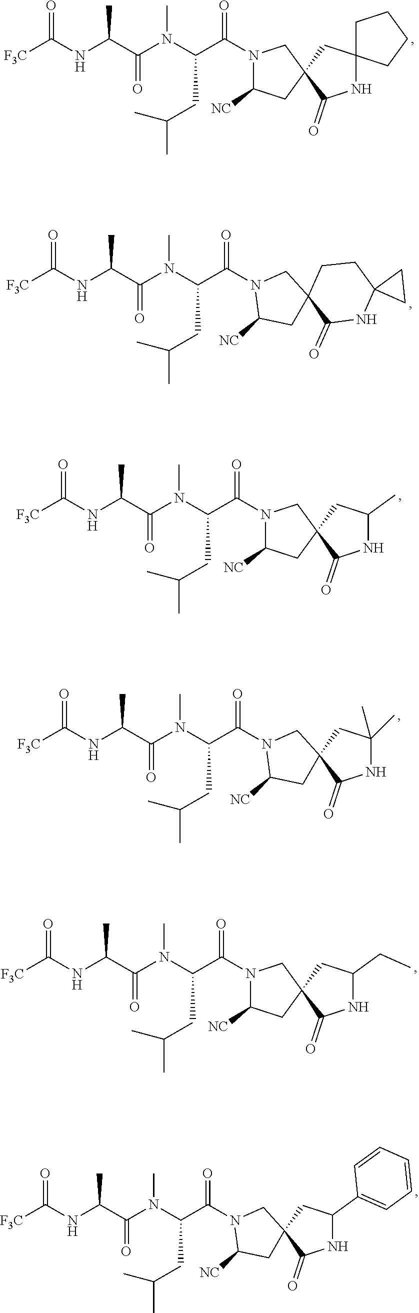

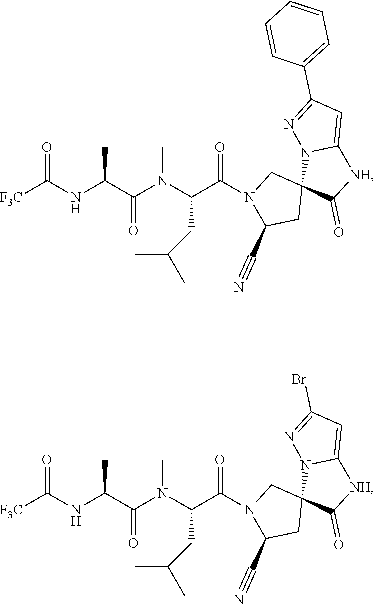 [SMILES: CC(C)C[C@@H](C(=O)N1C[C@]2(C[C@H]1C#N)C(=O)Nc1cc(-c3ccccc3)nn12)N(C)C(=O)[C@H](C)NC(=O)C(F)(F)F ; CC(C)C[C@@H](C(=O)N1C[C@]2(C[C@H]1C#N)C(=O)Nc1cc(Br)nn12)N(C)C(=O)[C@H](C)NC(=O)C(F)(F)F]

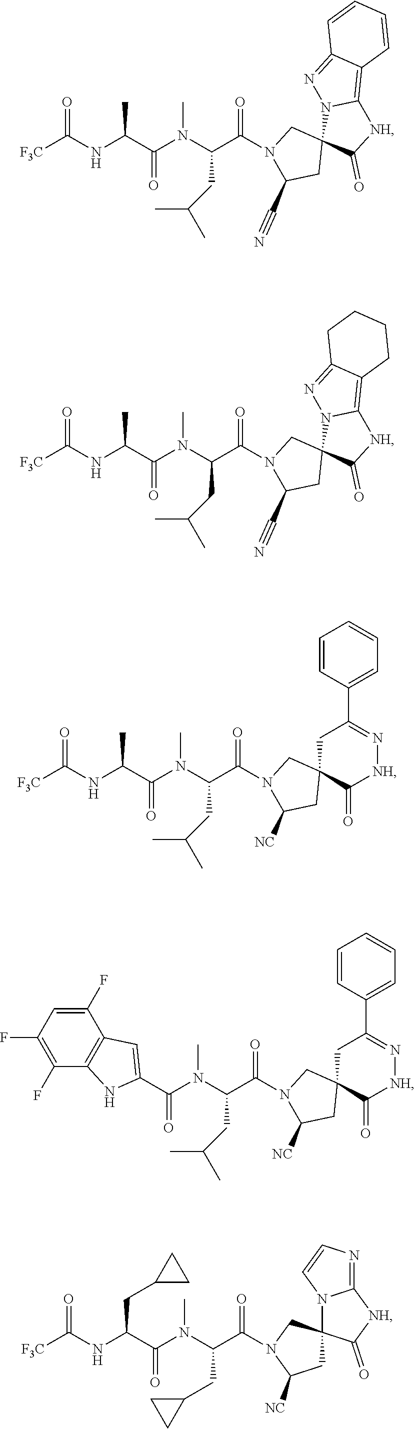

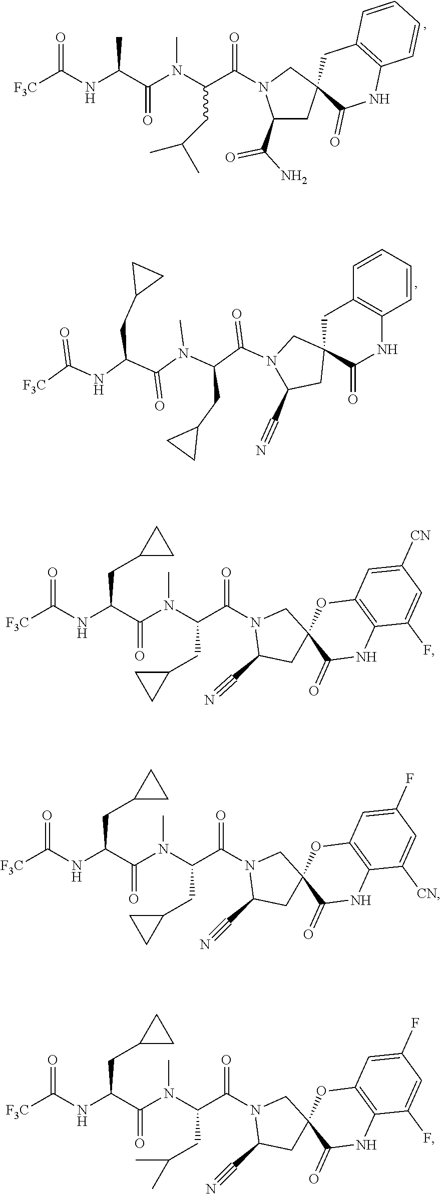 [SMILES: CC(C)CC(C(=O)N1C[C@]2(Cc3ccccc3NC2=O)C[C@H]1C(N)=O)N(C)C(=O)[C@H](C)NC(=O)C(F)(F)F ; CN(C(=O)[C@H](CC1CC1)NC(=O)C(F)(F)F)[C@H](CC1CC1)C(=O)N1C[C@]2(Cc3ccccc3NC2=O)C[C@H]1C#N ; CN(C(=O)[C@H](CC1CC1)NC(=O)C(F)(F)F)[C@@H](CC1CC1)C(=O)N1C[C@@]2(C[C@H]1C#N)Oc1cc(C#N)cc(F)c1NC2=O ; CN(C(=O)[C@H](CC1CC1)NC(=O)C(F)(F)F)[C@@H](CC1CC1)C(=O)N1C[C@@]2(C[C@H]1C#N)Oc1cc(F)cc(C#N)c1NC2=O ; CC(C)C[C@@H](C(=O)N1C[C@@]2(C[C@H]1C#N)Oc1cc(F)cc(F)c1NC2=O)N(C)C(=O)[C@H](CC1CC1)NC(=O)C(F)(F)F]

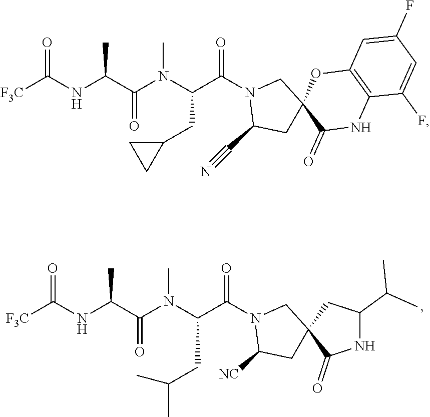

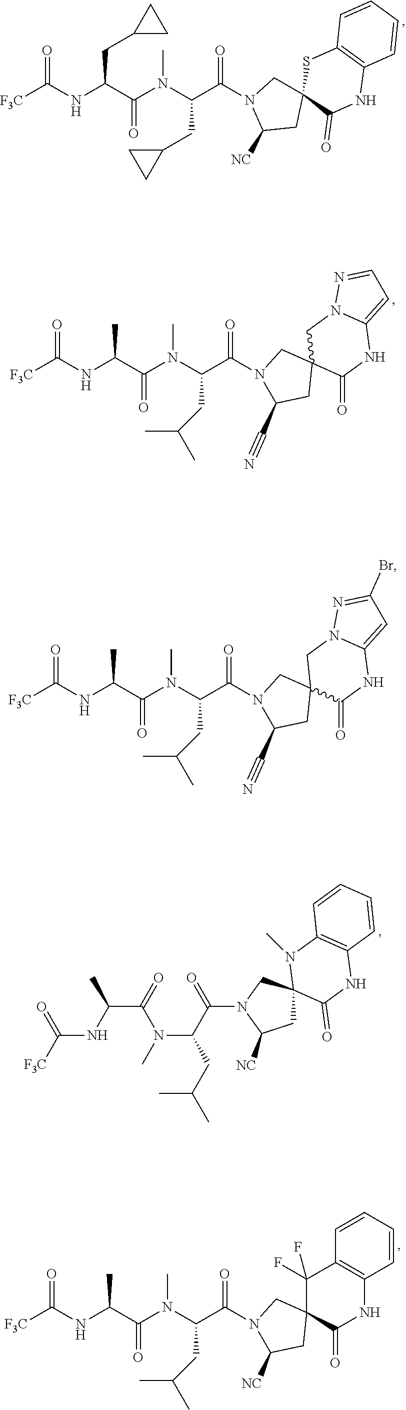 [SMILES: CN(C(=O)[C@H](CC1CC1)NC(=O)C(F)(F)F)[C@@H](CC1CC1)C(=O)N1C[C@@]2(C[C@H]1C#N)Sc1ccccc1NC2=O ; CC(C)C[C@@H](C(=O)N1CC2(C[C@H]1C#N)Cn1nccc1NC2=O)N(C)C(=O)[C@H](C)NC(=O)C(F)(F)F ; CC(C)C[C@@H](C(=O)N1CC2(C[C@H]1C#N)Cn1nc(Br)cc1NC2=O)N(C)C(=O)[C@H](C)NC(=O)C(F)(F)F ; CC(C)C[C@@H](C(=O)N1C[C@]2(C[C@H]1C#N)C(=O)Nc1ccccc1N2C)N(C)C(=O)[C@H](C)NC(=O)C(F)(F)F ; CC(C)C[C@@H](C(=O)N1C[C@]2(C[C@H]1C#N)C(=O)Nc1ccccc1C2(F)F)N(C)C(=O)[C@H](C)NC(=O)C(F)(F)F]

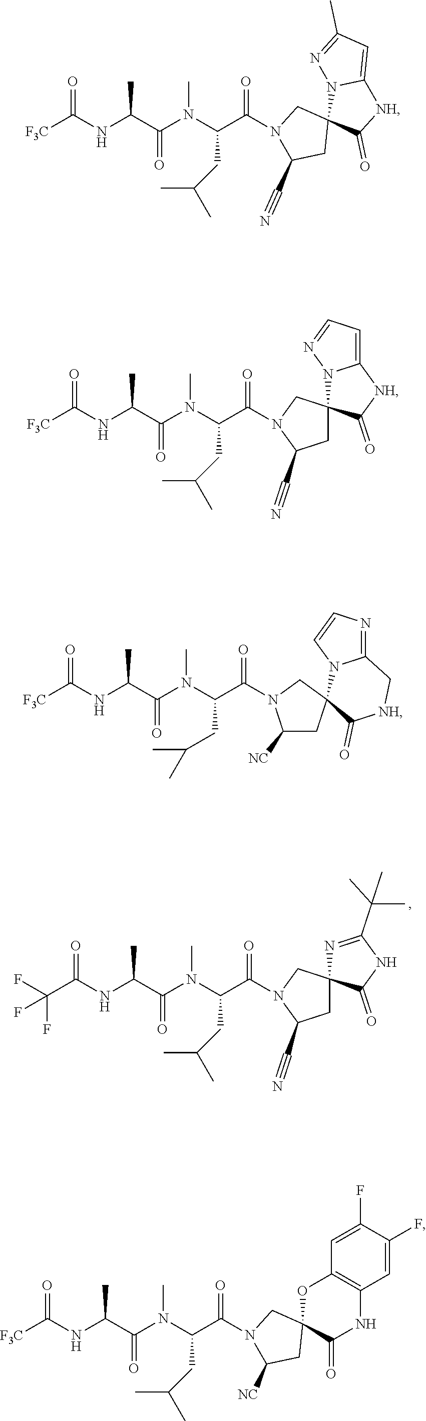

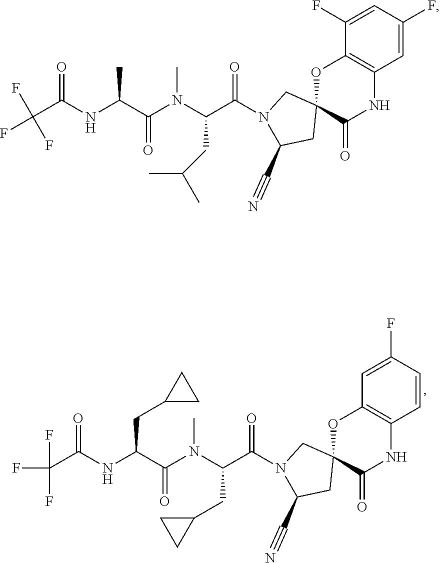 [SMILES: CC(C)C[C@@H](C(=O)N1C[C@@]2(C[C@H]1C#N)Oc1c(F)cc(F)cc1NC2=O)N(C)C(=O)[C@H](C)NC(=O)C(F)(F)F ; CN(C(=O)[C@H](CC1CC1)NC(=O)C(F)(F)F)[C@@H](CC1CC1)C(=O)N1C[C@@]2(C[C@H]1C#N)Oc1cc(F)ccc1NC2=O]

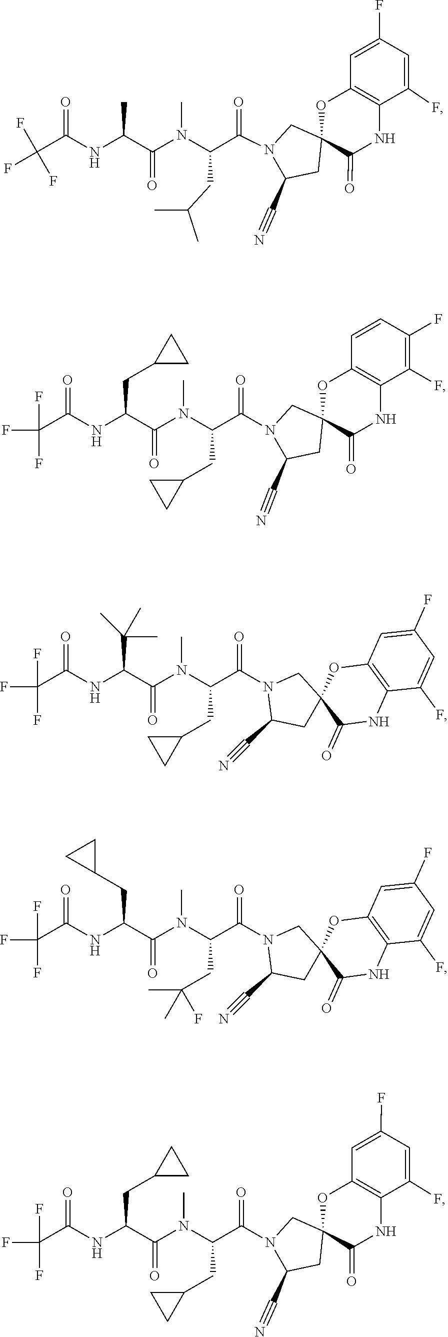 [SMILES: CC(C)C[C@@H](C(=O)N1C[C@@]2(C[C@H]1C#N)Oc1cc(F)cc(F)c1NC2=O)N(C)C(=O)[C@H](C)NC(=O)C(F)(F)F ; CN(C(=O)[C@H](CC1CC1)NC(=O)C(F)(F)F)[C@@H](CC1CC1)C(=O)N1C[C@@]2(C[C@H]1C#N)Oc1ccc(F)c(F)c1NC2=O ; CN(C(=O)[C@@H](NC(=O)C(F)(F)F)C(C)(C)C)[C@@H](CC1CC1)C(=O)N1C[C@@]2(C[C@H]1C#N)Oc1cc(F)cc(F)c1NC2=O ; CN(C(=O)[C@H](CC1CC1)NC(=O)C(F)(F)F)[C@@H](CC(C)(C)F)C(=O)N1C[C@@]2(C[C@H]1C#N)Oc1cc(F)cc(F)c1NC2=O ; CN(C(=O)[C@H](CC1CC1)NC(=O)C(F)(F)F)[C@@H](CC1CC1)C(=O)N1C[C@@]2(C[C@H]1C#N)Oc1cc(F)cc(F)c1NC2=O]

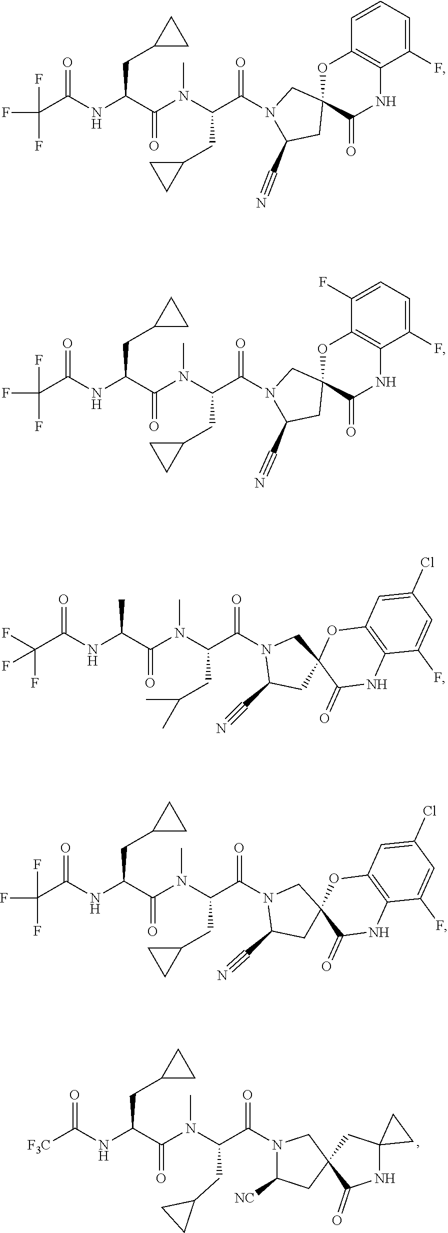 [SMILES: CN(C(=O)[C@H](CC1CC1)NC(=O)C(F)(F)F)[C@@H](CC1CC1)C(=O)N1C[C@@]2(C[C@H]1C#N)Oc1cccc(F)c1NC2=O ; CN(C(=O)[C@H](CC1CC1)NC(=O)C(F)(F)F)[C@@H](CC1CC1)C(=O)N1C[C@@]2(C[C@H]1C#N)Oc1c(F)ccc(F)c1NC2=O ; CC(C)C[C@@H](C(=O)N1C[C@@]2(C[C@H]1C#N)Oc1cc(Cl)cc(F)c1NC2=O)N(C)C(=O)[C@H](C)NC(=O)C(F)(F)F ; CN(C(=O)[C@H](CC1CC1)NC(=O)C(F)(F)F)[C@@H](CC1CC1)C(=O)N1C[C@@]2(C[C@H]1C#N)Oc1cc(Cl)cc(F)c1NC2=O ; CN(C(=O)[C@H](CC1CC1)NC(=O)C(F)(F)F)[C@@H](CC1CC1)C(=O)N1C[C@@]2(C[C@H]1C#N)CC1(CC1)NC2=O]

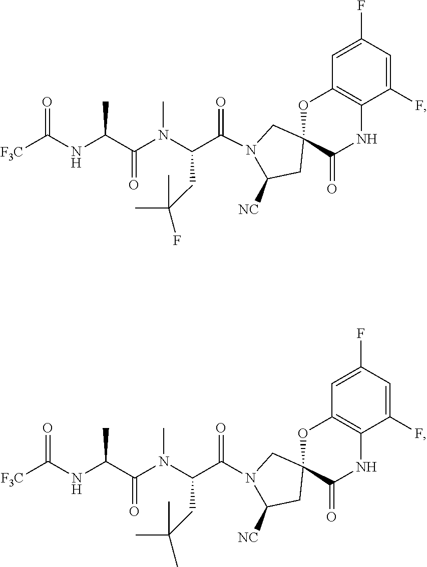 [SMILES: C[C@H](NC(=O)C(F)(F)F)C(=O)N(C)[C@@H](CC(C)(C)F)C(=O)N1C[C@@]2(C[C@H]1C#N)Oc1cc(F)cc(F)c1NC2=O ; C[C@H](NC(=O)C(F)(F)F)C(=O)N(C)[C@@H](CC(C)(C)C)C(=O)N1C[C@@]2(C[C@H]1C#N)Oc1cc(F)cc(F)c1NC2=O]

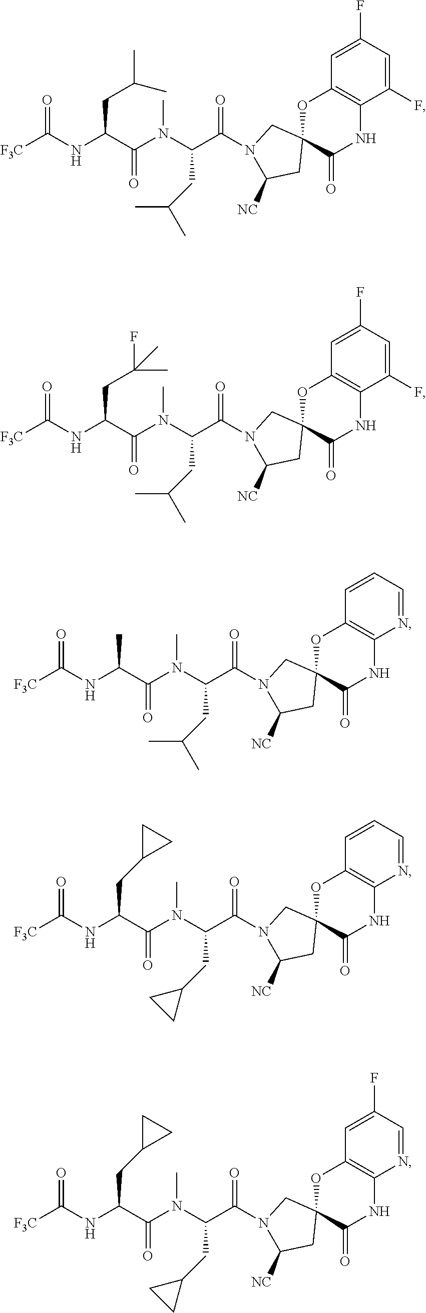 [SMILES: CC(C)C[C@H](NC(=O)C(F)(F)F)C(=O)N(C)[C@@H](CC(C)C)C(=O)N1C[C@@]2(C[C@H]1C#N)Oc1cc(F)cc(F)c1NC2=O ; CC(C)C[C@@H](C(=O)N1C[C@@]2(C[C@H]1C#N)Oc1cc(F)cc(F)c1NC2=O)N(C)C(=O)[C@H](CC(C)(C)F)NC(=O)C(F)(F)F ; CC(C)C[C@@H](C(=O)N1C[C@@]2(C[C@H]1C#N)Oc1cccnc1NC2=O)N(C)C(=O)[C@H](C)NC(=O)C(F)(F)F ; CN(C(=O)[C@H](CC1CC1)NC(=O)C(F)(F)F)[C@@H](CC1CC1)C(=O)N1C[C@@]2(C[C@H]1C#N)Oc1cccnc1NC2=O ; CN(C(=O)[C@H](CC1CC1)NC(=O)C(F)(F)F)[C@@H](CC1CC1)C(=O)N1C[C@@]2(C[C@H]1C#N)Oc1cc(F)cnc1NC2=O]

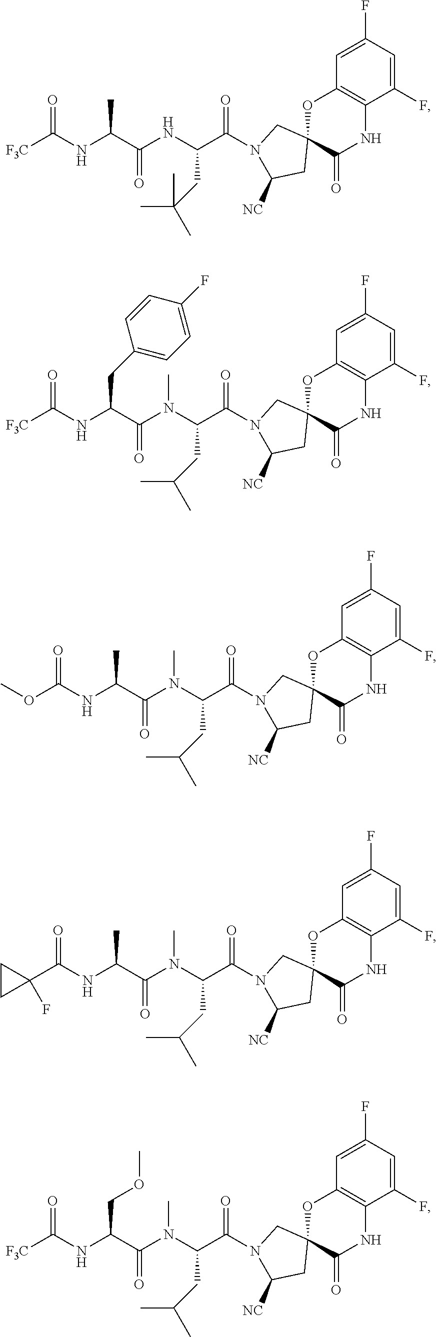 [SMILES: C[C@H](NC(=O)C(F)(F)F)C(=O)N[C@@H](CC(C)(C)C)C(=O)N1C[C@@]2(C[C@H]1C#N)Oc1cc(F)cc(F)c1NC2=O ; CC(C)C[C@@H](C(=O)N1C[C@@]2(C[C@H]1C#N)Oc1cc(F)cc(F)c1NC2=O)N(C)C(=O)[C@H](Cc1ccc(F)cc1)NC(=O)C(F)(F)F ; COC(=O)N[C@@H](C)C(=O)N(C)[C@@H](CC(C)C)C(=O)N1C[C@@]2(C[C@H]1C#N)Oc1cc(F)cc(F)c1NC2=O ; CC(C)C[C@@H](C(=O)N1C[C@@]2(C[C@H]1C#N)Oc1cc(F)cc(F)c1NC2=O)N(C)C(=O)[C@H](C)NC(=O)C1(F)CC1 ; COC[C@H](NC(=O)C(F)(F)F)C(=O)N(C)[C@@H](CC(C)C)C(=O)N1C[C@@]2(C[C@H]1C#N)Oc1cc(F)cc(F)c1NC2=O]

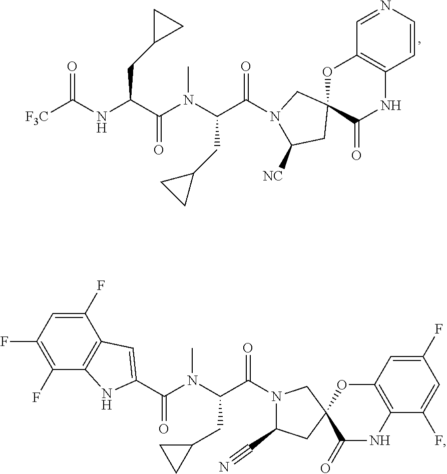 [SMILES: CN(C(=O)[C@H](CC1CC1)NC(=O)C(F)(F)F)[C@@H](CC1CC1)C(=O)N1C[C@@]2(C[C@H]1C#N)Oc1cnccc1NC2=O ; CN(C(=O)c1cc2c(F)cc(F)c(F)c2[nH]1)[C@@H](CC1CC1)C(=O)N1C[C@@]2(C[C@H]1C#N)Oc1cc(F)cc(F)c1NC2=O]

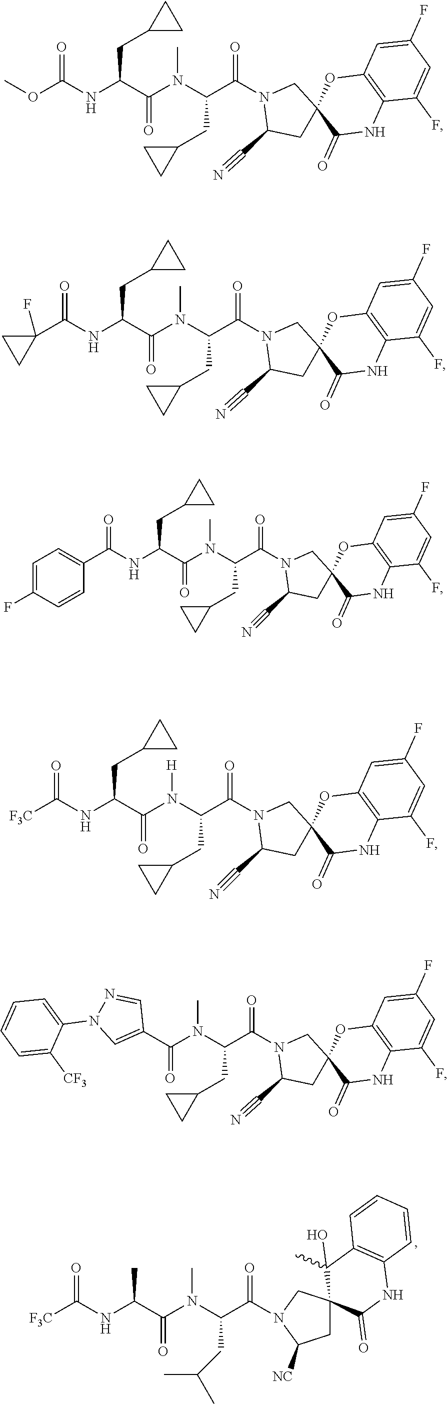

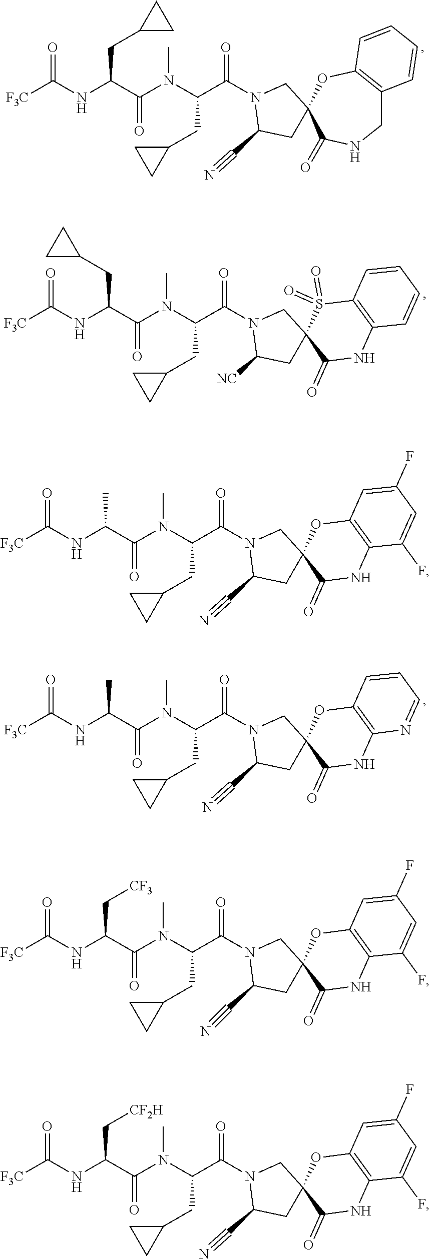

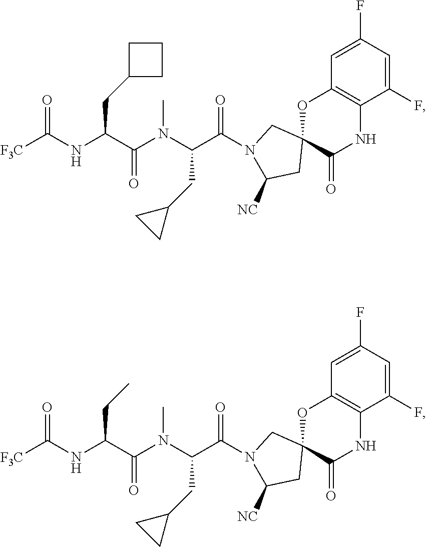 [SMILES: CN(C(=O)[C@H](CC1CCC1)NC(=O)C(F)(F)F)[C@@H](CC1CC1)C(=O)N1C[C@@]2(C[C@H]1C#N)Oc1cc(F)cc(F)c1NC2=O ; CC[C@H](NC(=O)C(F)(F)F)C(=O)N(C)[C@@H](CC1CC1)C(=O)N1C[C@@]2(C[C@H]1C#N)Oc1cc(F)cc(F)c1NC2=O]

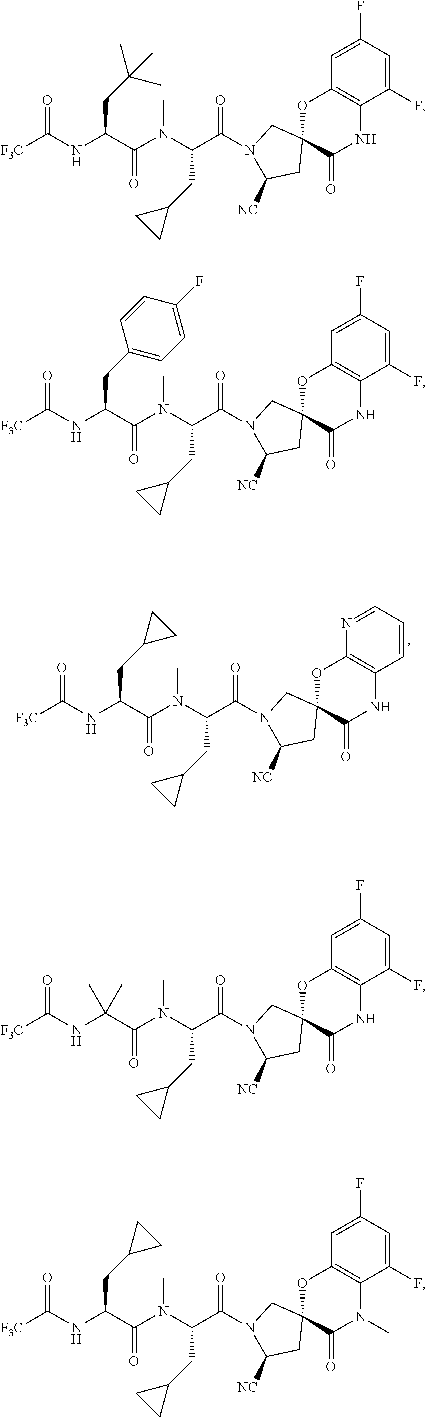 [SMILES: CN(C(=O)[C@H](CC(C)(C)C)NC(=O)C(F)(F)F)[C@@H](CC1CC1)C(=O)N1C[C@@]2(C[C@H]1C#N)Oc1cc(F)cc(F)c1NC2=O ; CN(C(=O)[C@H](Cc1ccc(F)cc1)NC(=O)C(F)(F)F)[C@@H](CC1CC1)C(=O)N1C[C@@]2(C[C@H]1C#N)Oc1cc(F)cc(F)c1NC2=O ; CN(C(=O)[C@H](CC1CC1)NC(=O)C(F)(F)F)[C@@H](CC1CC1)C(=O)N1C[C@@]2(C[C@H]1C#N)Oc1ncccc1NC2=O ; CN(C(=O)C(C)(C)NC(=O)C(F)(F)F)[C@@H](CC1CC1)C(=O)N1C[C@@]2(C[C@H]1C#N)Oc1cc(F)cc(F)c1NC2=O ; CN1C(=O)[C@]2(C[C@@H](C#N)N(C(=O)[C@H](CC3CC3)N(C)C(=O)[C@H](CC3CC3)NC(=O)C(F)(F)F)C2)Oc2cc(F)cc(F)c21]

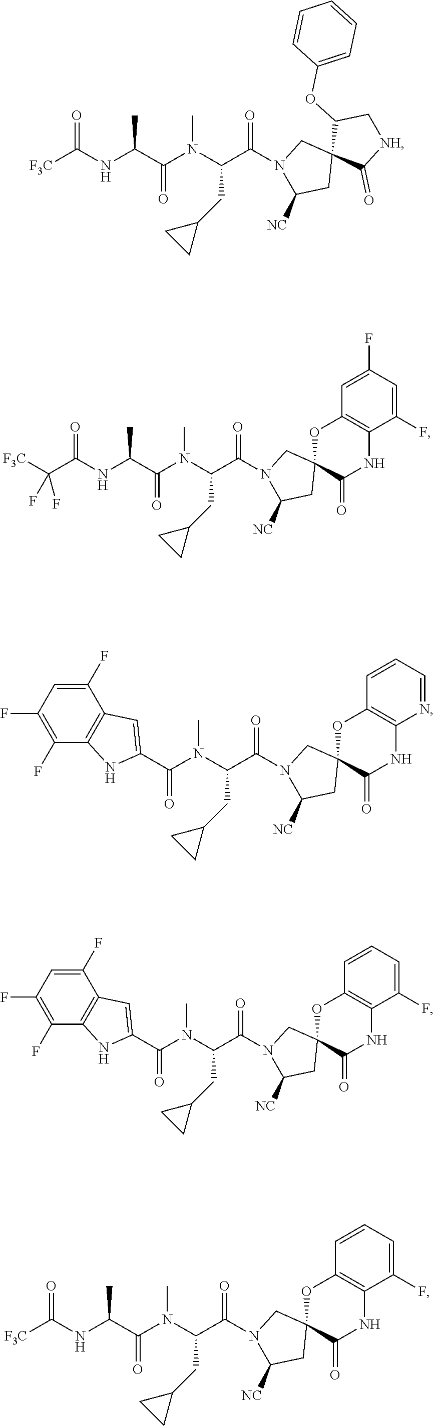

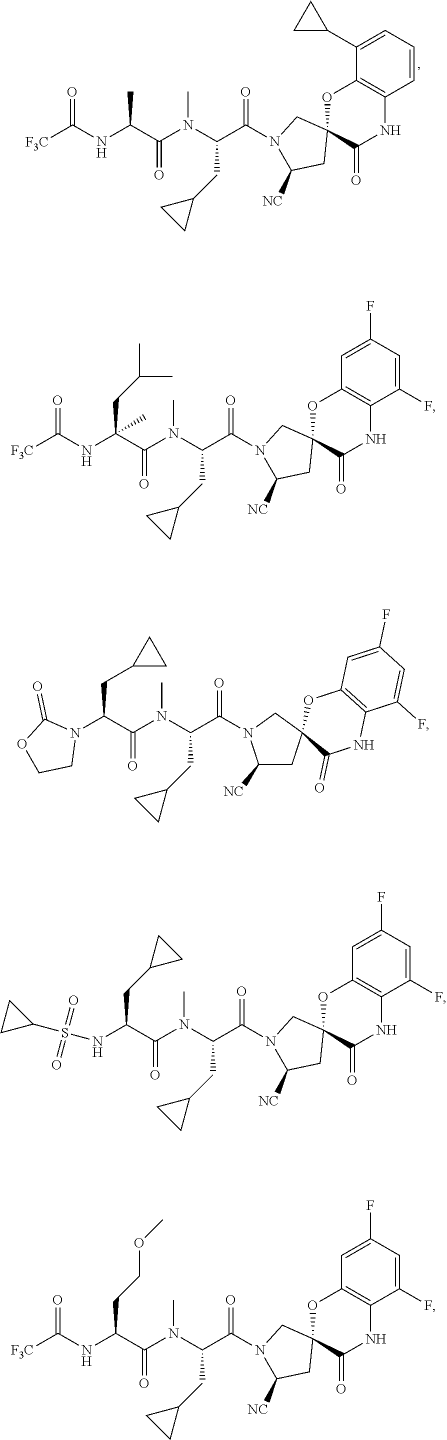 [SMILES: C[C@H](NC(=O)C(F)(F)F)C(=O)N(C)[C@@H](CC1CC1)C(=O)N1C[C@@]2(C[C@H]1C#N)Oc1c(cccc1C1CC1)NC2=O ; CC(C)C[C@](C)(NC(=O)C(F)(F)F)C(=O)N(C)[C@@H](CC1CC1)C(=O)N1C[C@@]2(C[C@H]1C#N)Oc1cc(F)cc(F)c1NC2=O ; CN(C(=O)[C@H](CC1CC1)N1CCOC1=O)[C@@H](CC1CC1)C(=O)N1C[C@@]2(C[C@H]1C#N)Oc1cc(F)cc(F)c1NC2=O ; CN(C(=O)[C@H](CC1CC1)NS(=O)(=O)C1CC1)[C@@H](CC1CC1)C(=O)N1C[C@@]2(C[C@H]1C#N)Oc1cc(F)cc(F)c1NC2=O ; COCC[C@H](NC(=O)C(F)(F)F)C(=O)N(C)[C@@H](CC1CC1)C(=O)N1C[C@@]2(C[C@H]1C#N)Oc1cc(F)cc(F)c1NC2=O]

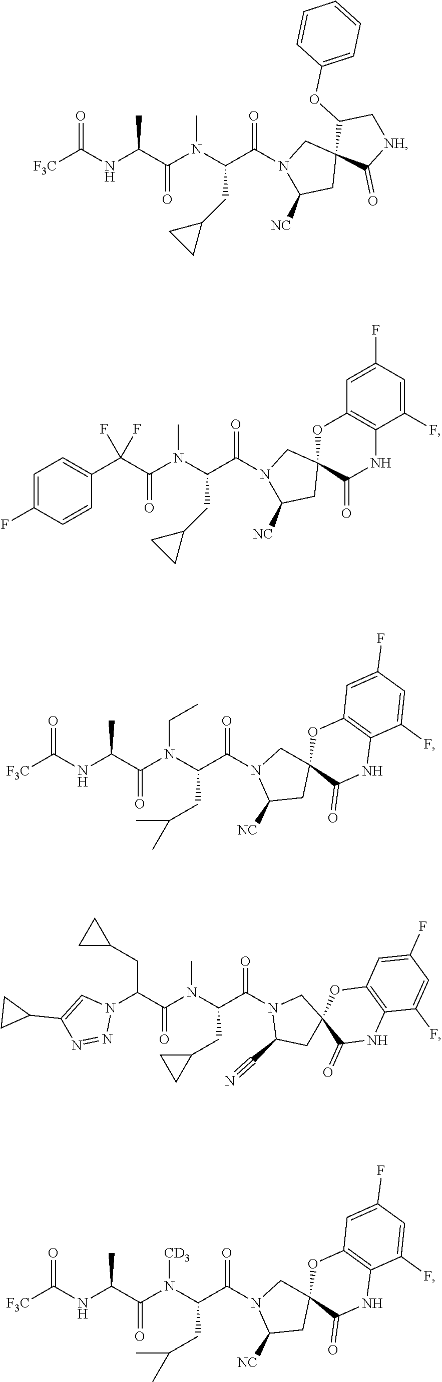

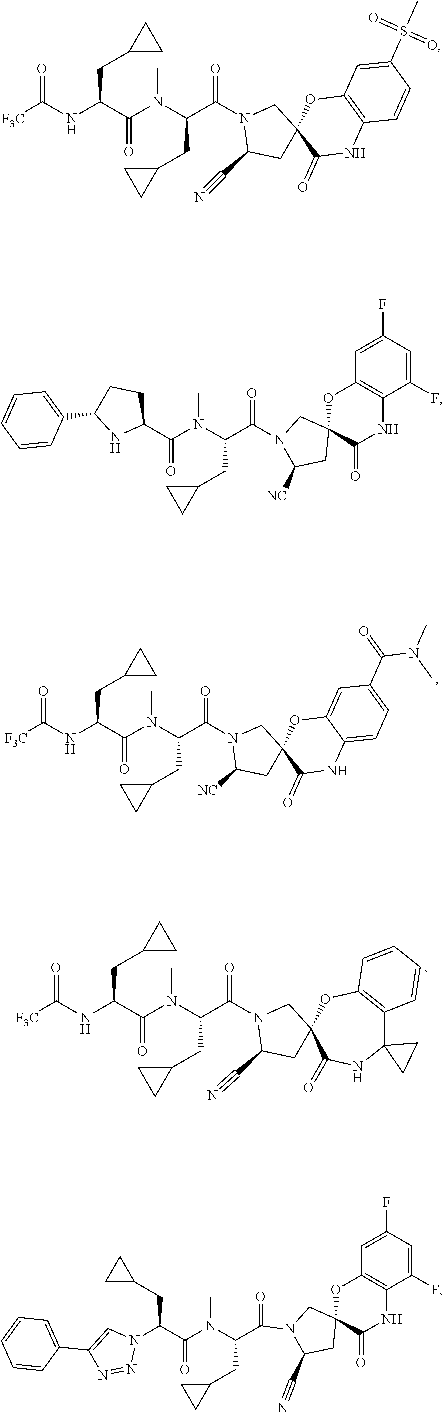

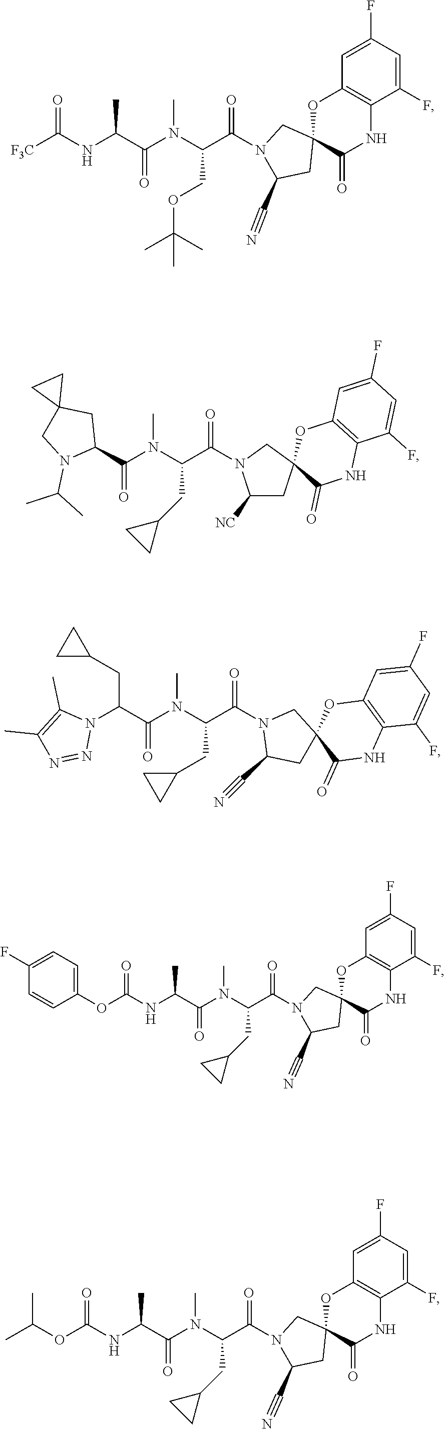 [SMILES: C[C@H](NC(=O)C(F)(F)F)C(=O)N(C)[C@@H](COC(C)(C)C)C(=O)N1C[C@@]2(C[C@H]1C#N)Oc1cc(F)cc(F)c1NC2=O ; CC(C)N1CC2(CC2)C[C@H]1C(=O)N(C)[C@@H](CC1CC1)C(=O)N1C[C@@]2(C[C@H]1C#N)Oc1cc(F)cc(F)c1NC2=O ; Cc1nnn(C(CC2CC2)C(=O)N(C)[C@@H](CC2CC2)C(=O)N2C[C@@]3(C[C@H]2C#N)Oc2cc(F)cc(F)c2NC3=O)c1C ; C[C@H](NC(=O)Oc1ccc(F)cc1)C(=O)N(C)[C@@H](CC1CC1)C(=O)N1C[C@@]2(C[C@H]1C#N)Oc1cc(F)cc(F)c1NC2=O ; CC(C)OC(=O)N[C@@H](C)C(=O)N(C)[C@@H](CC1CC1)C(=O)N1C[C@@]2(C[C@H]1C#N)Oc1cc(F)cc(F)c1NC2=O]

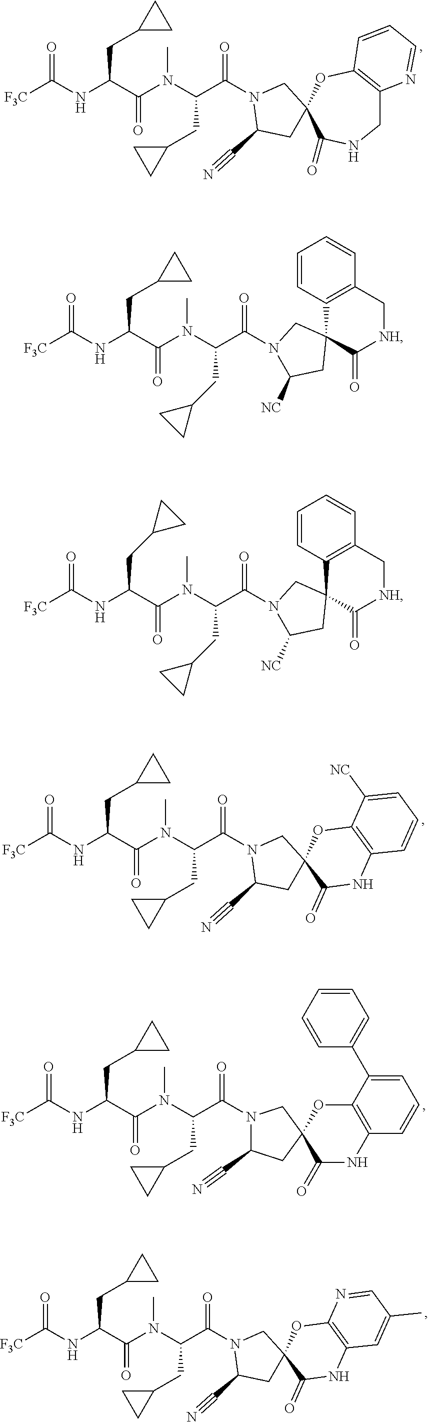

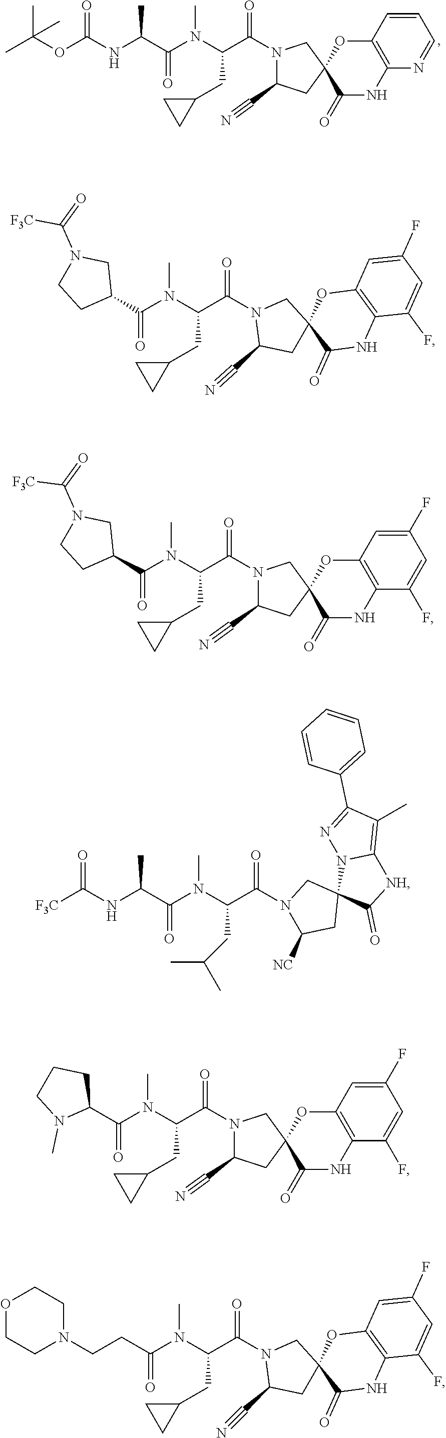

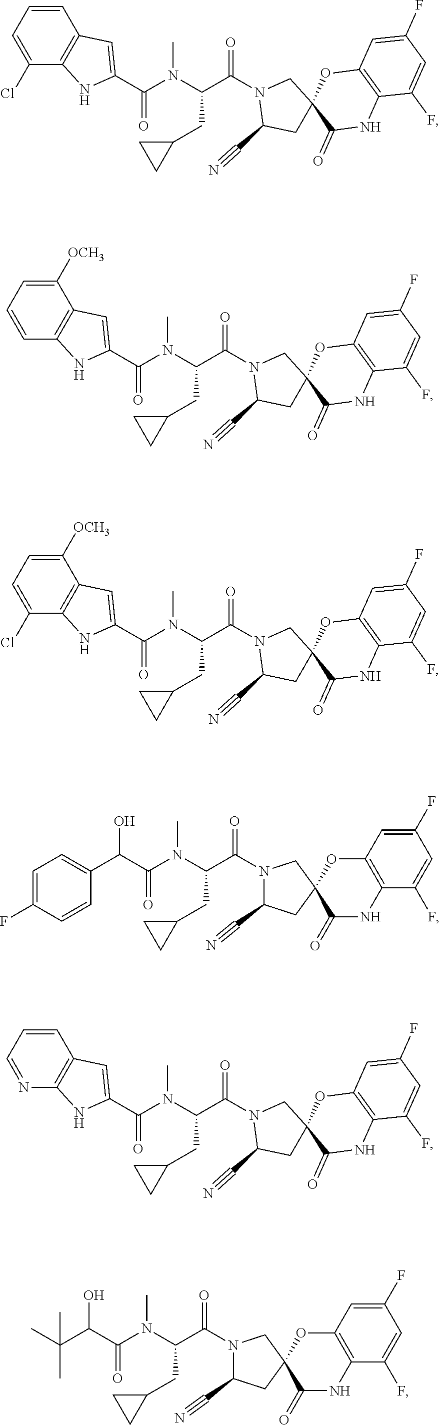

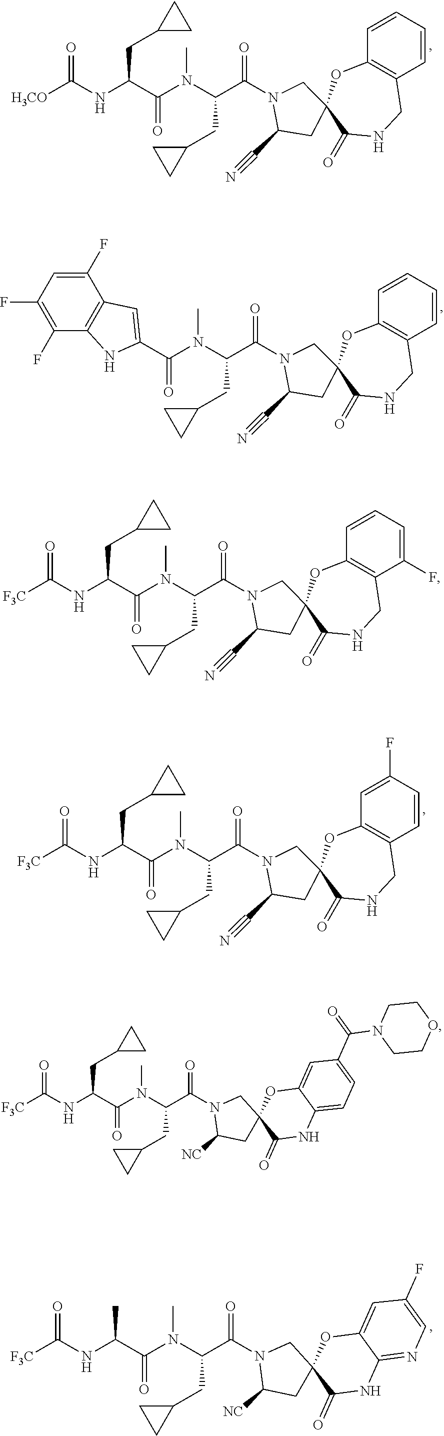

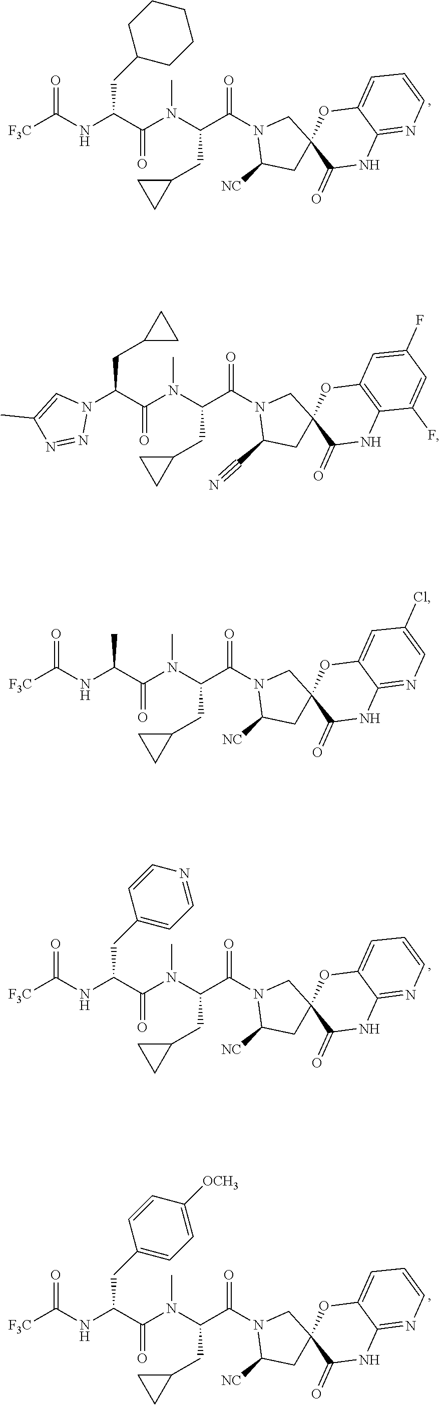 [SMILES: CN(C(=O)[C@@H](CC1CCCCC1)NC(=O)C(F)(F)F)[C@@H](CC1CC1)C(=O)N1C[C@@]2(C[C@H]1C#N)Oc1cccnc1NC2=O ; Cc1cn([C@@H](CC2CC2)C(=O)N(C)[C@@H](CC2CC2)C(=O)N2C[C@@]3(C[C@H]2C#N)Oc2cc(F)cc(F)c2NC3=O)nn1 ; C[C@H](NC(=O)C(F)(F)F)C(=O)N(C)[C@@H](CC1CC1)C(=O)N1C[C@@]2(C[C@H]1C#N)Oc1cc(Cl)cnc1NC2=O ; CN(C(=O)[C@@H](Cc1ccncc1)NC(=O)C(F)(F)F)[C@@H](CC1CC1)C(=O)N1C[C@@]2(C[C@H]1C#N)Oc1cccnc1NC2=O ; COc1ccc(C[C@@H](NC(=O)C(F)(F)F)C(=O)N(C)[C@@H](CC2CC2)C(=O)N2C[C@@]3(C[C@H]2C#N)Oc2cccnc2NC3=O)cc1]

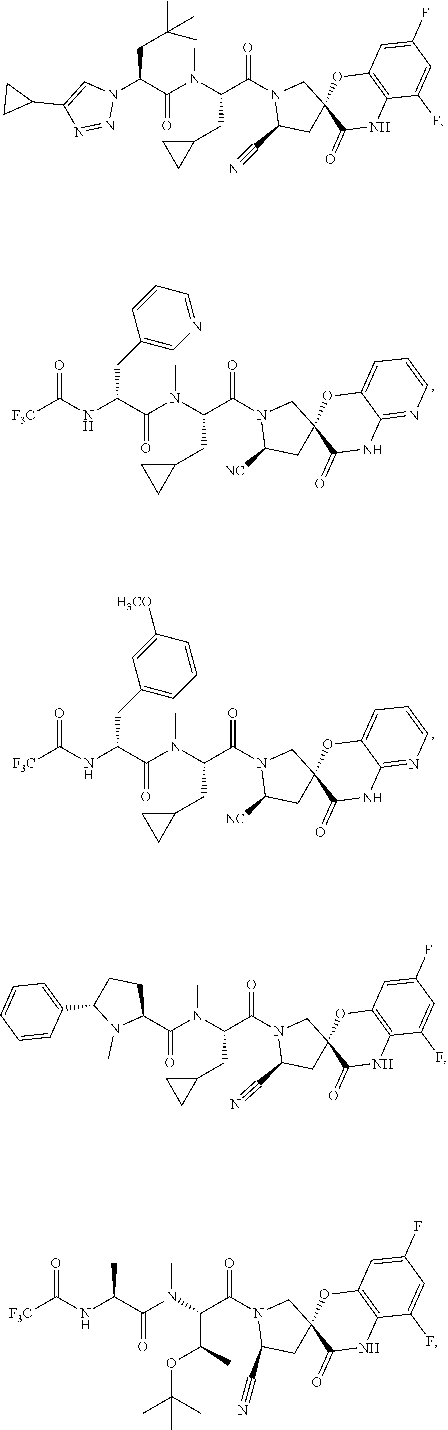

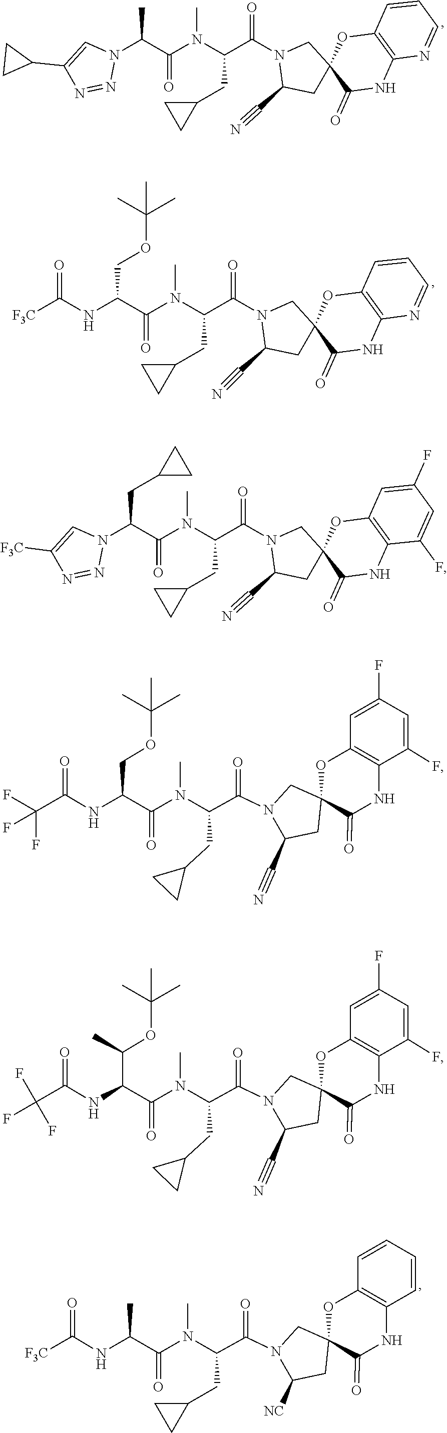

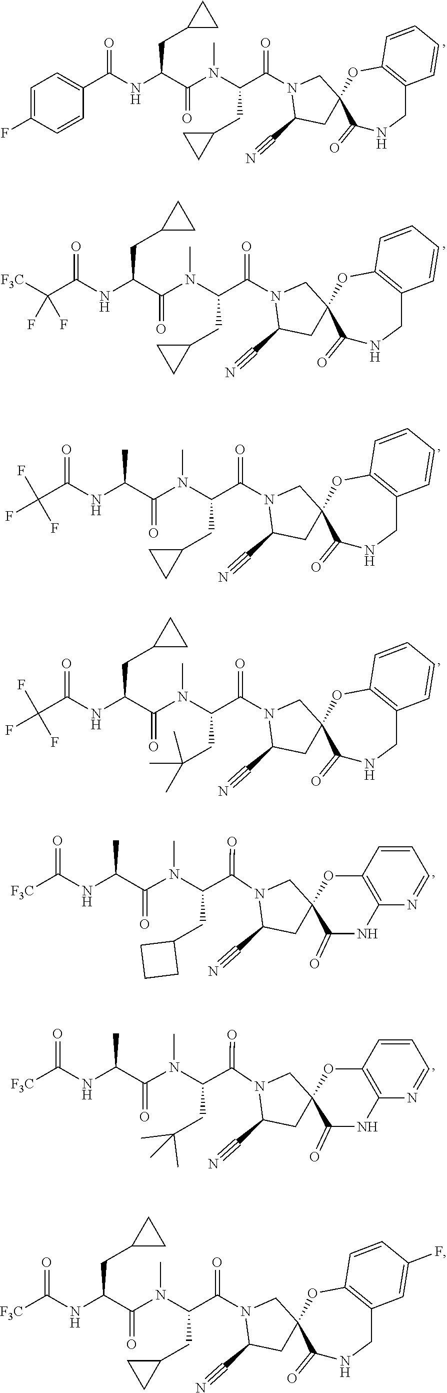

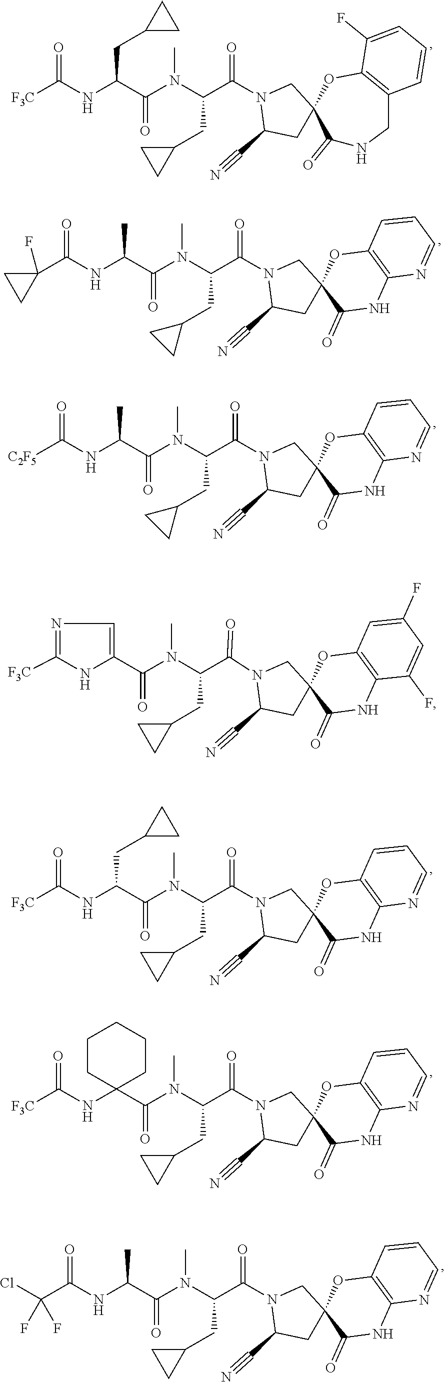

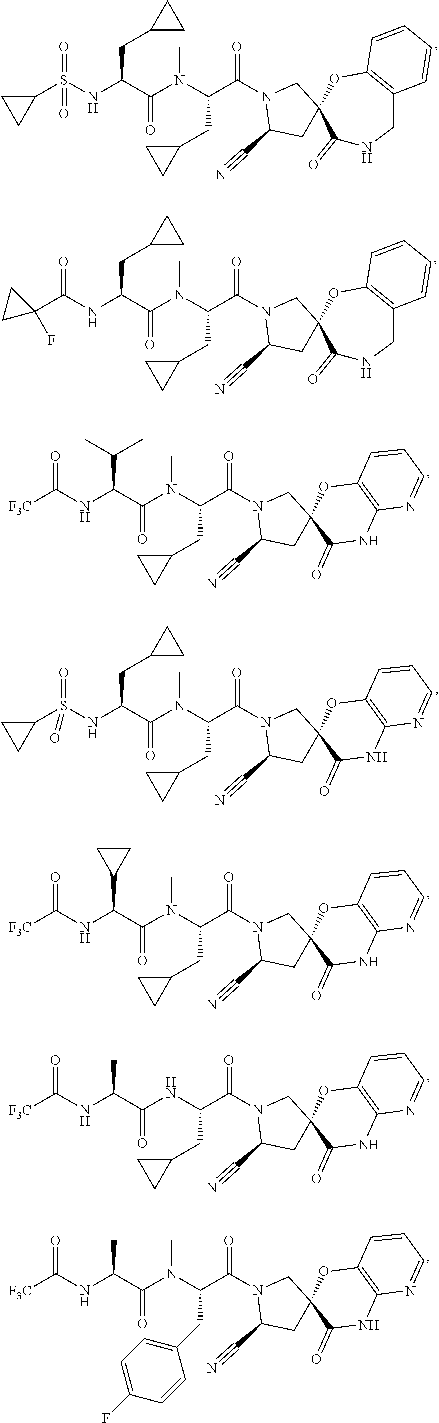

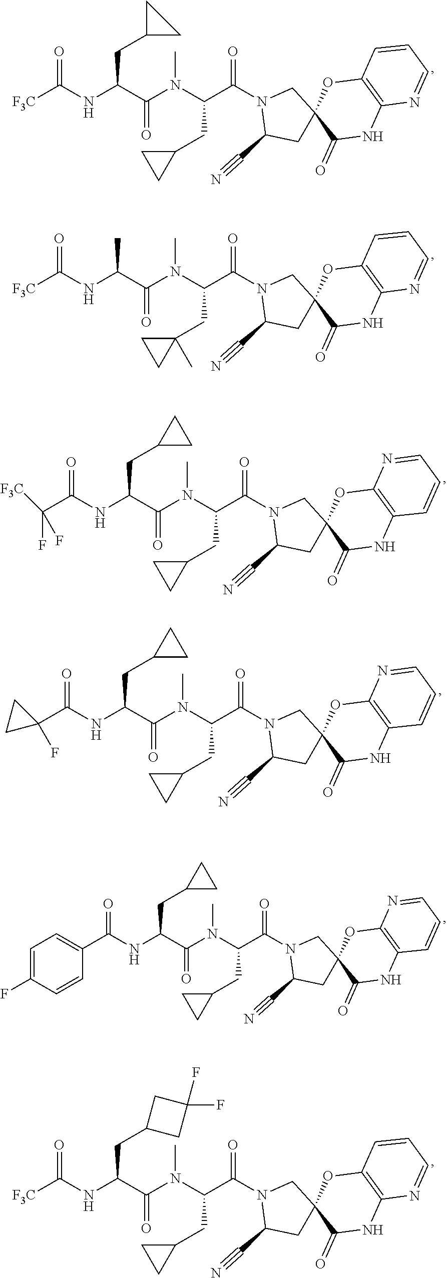

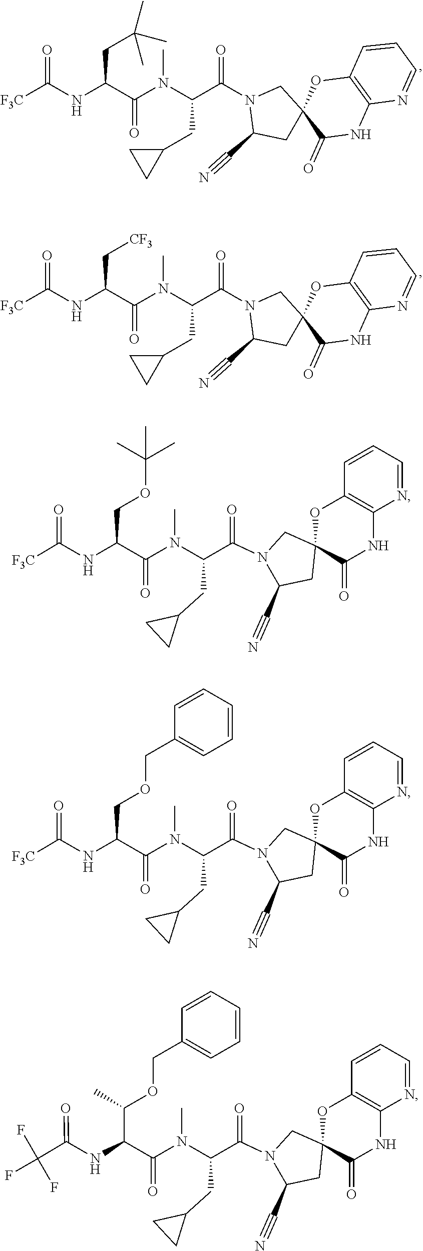

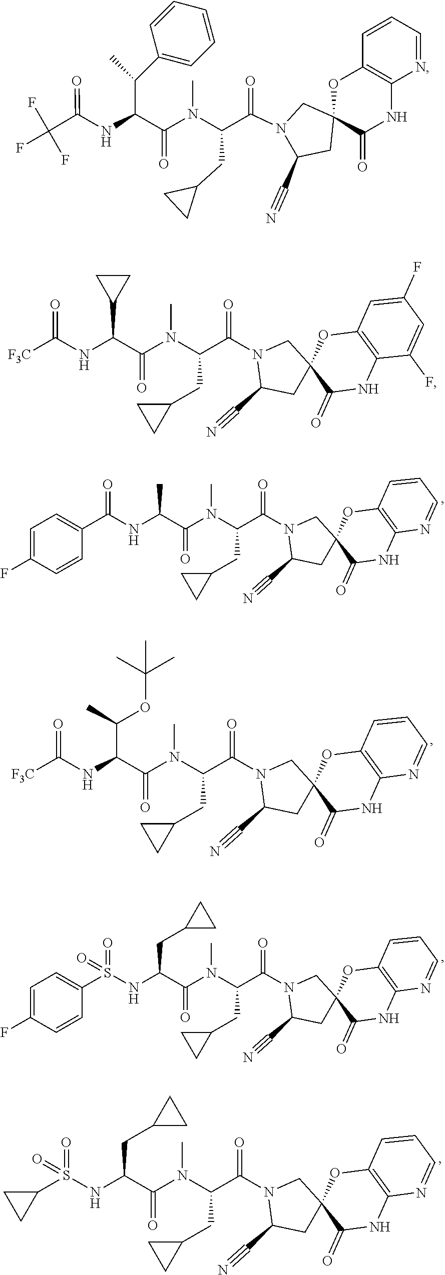

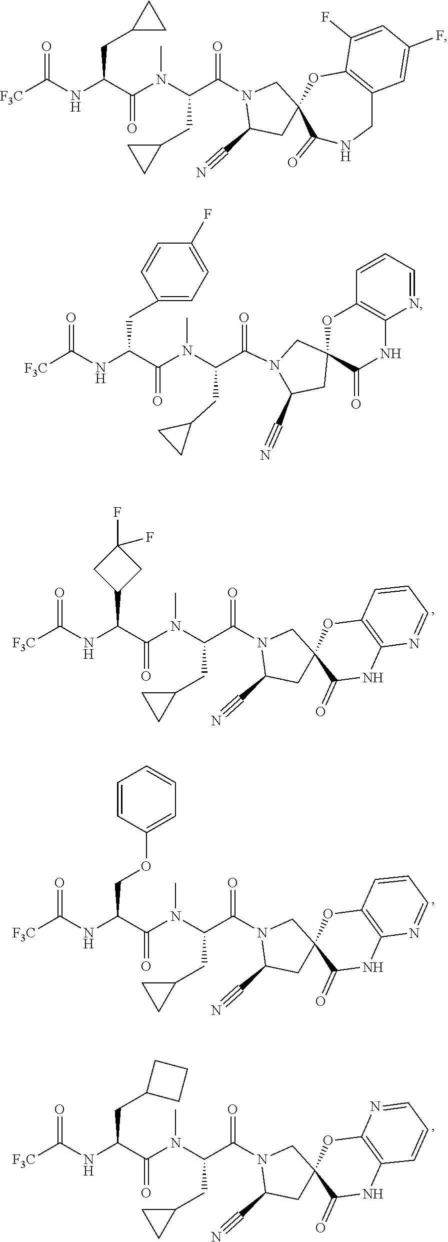

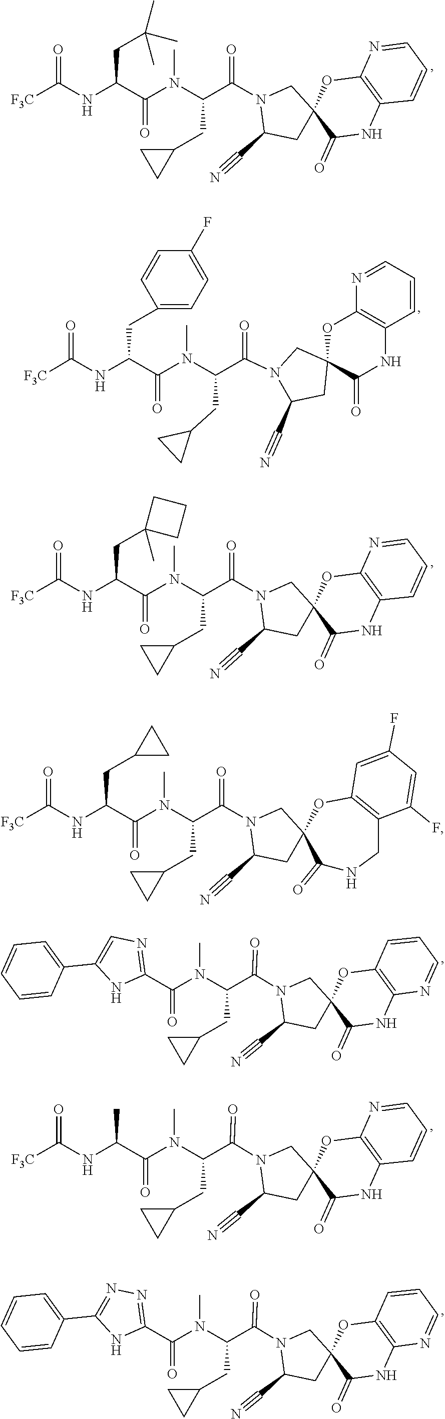

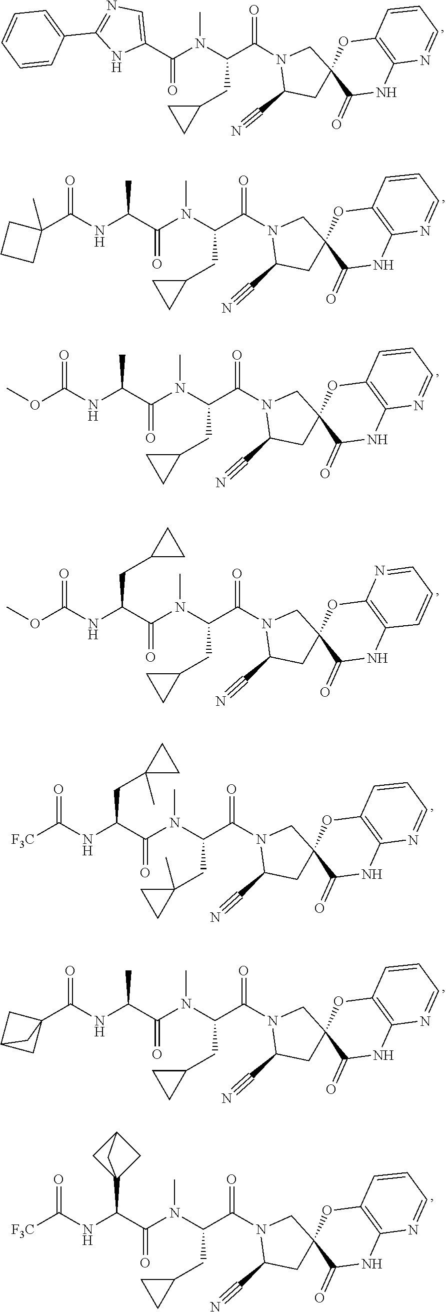

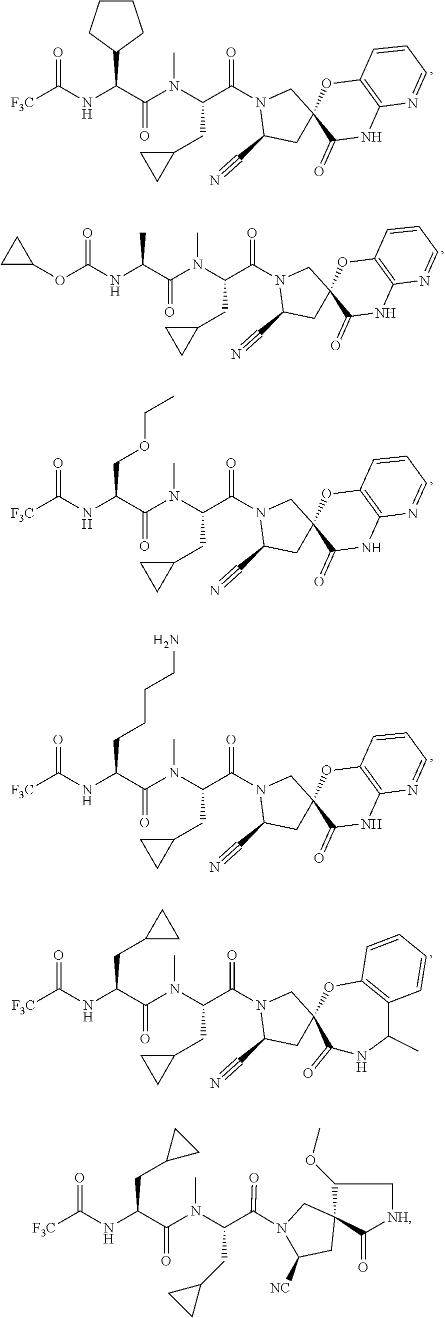

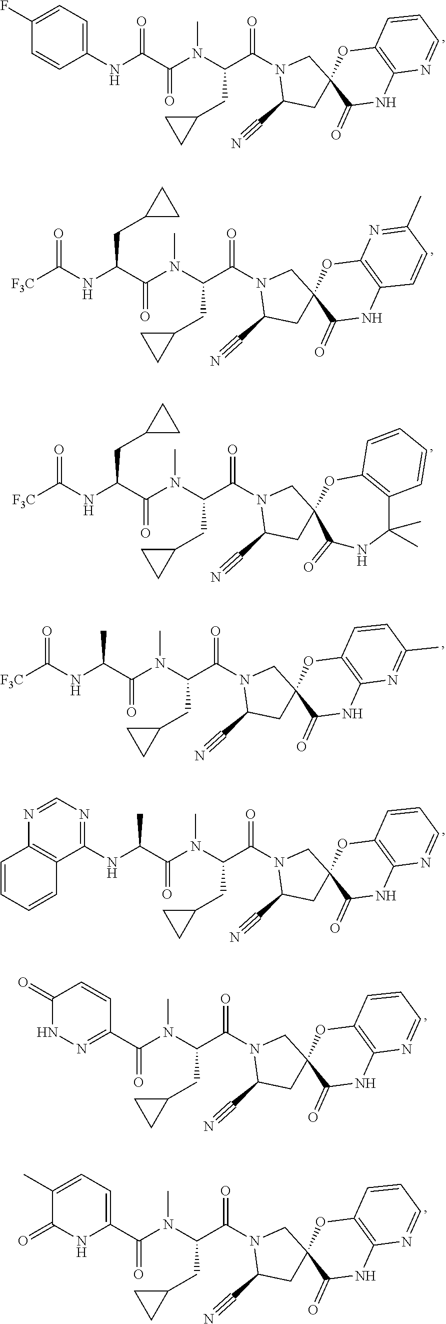

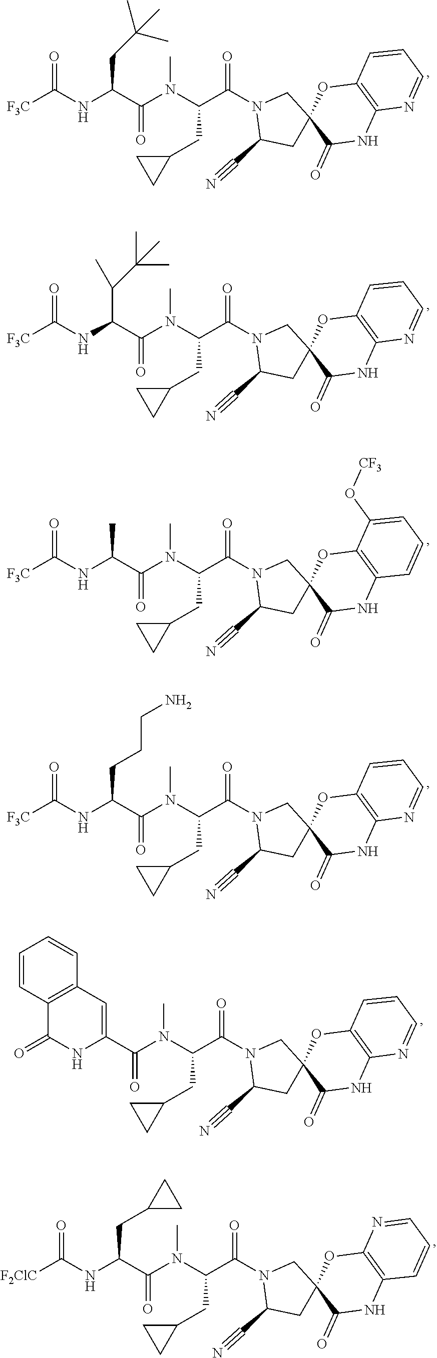

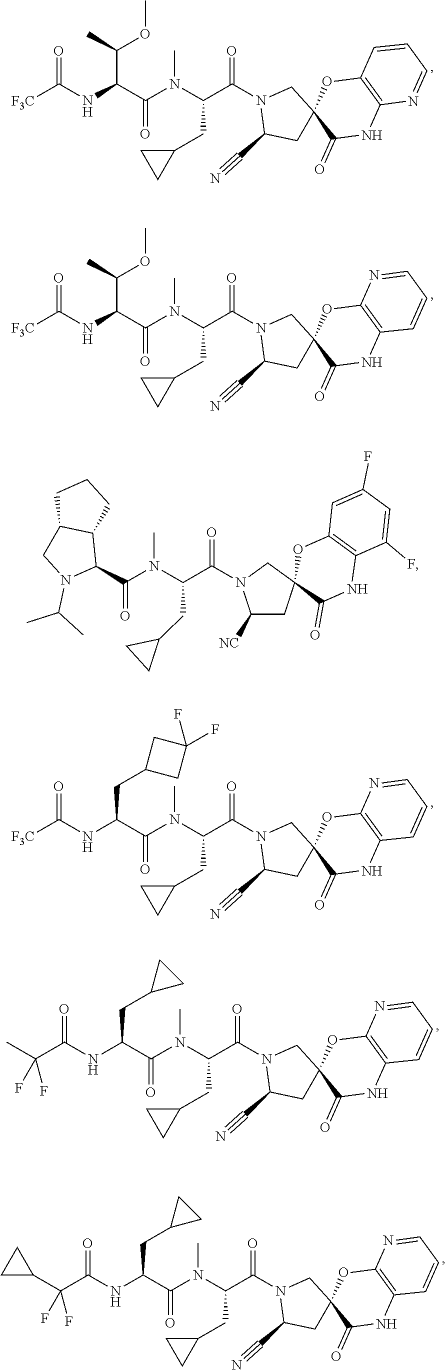

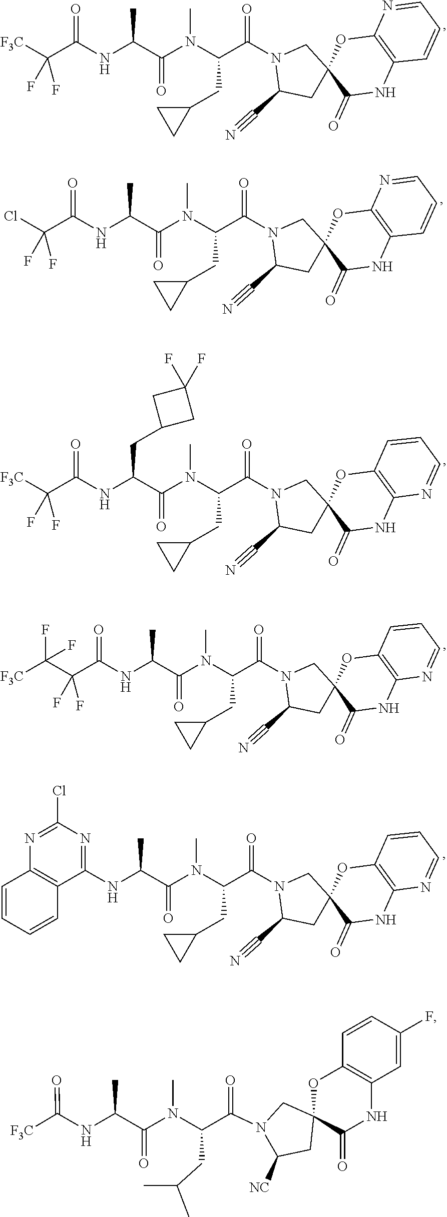

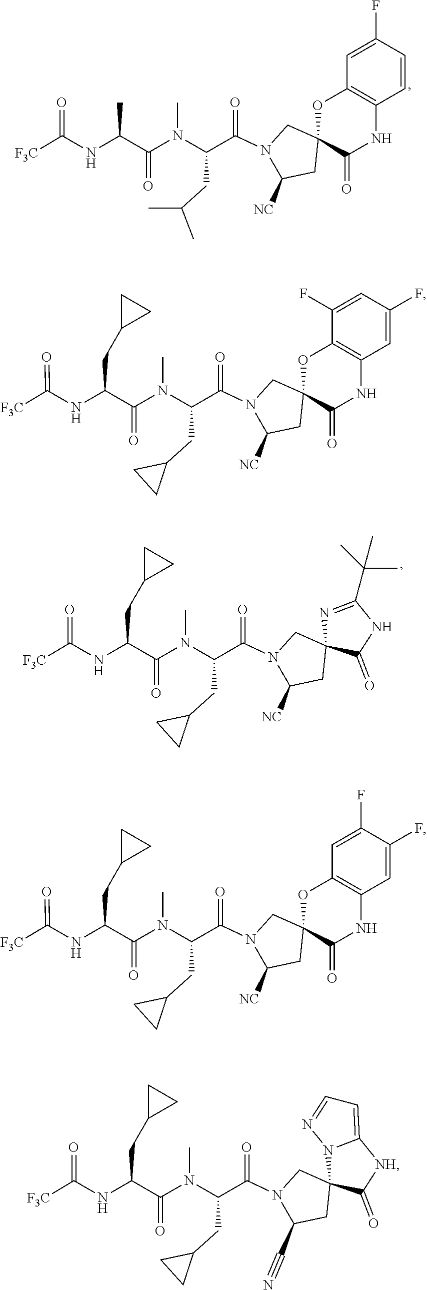 [SMILES: CC(C)C[C@@H](C(=O)N1C[C@@]2(C[C@H]1C#N)Oc1cc(F)ccc1NC2=O)N(C)C(=O)[C@H](C)NC(=O)C(F)(F)F ; CN(C(=O)[C@H](CC1CC1)NC(=O)C(F)(F)F)[C@@H](CC1CC1)C(=O)N1C[C@@]2(C[C@H]1C#N)Oc1c(F)cc(F)cc1NC2=O ; CN(C(=O)[C@H](CC1CC1)NC(=O)C(F)(F)F)[C@@H](CC1CC1)C(=O)N1C[C@@]2(C[C@H]1C#N)N=C(C(C)(C)C)NC2=O ; CN(C(=O)[C@H](CC1CC1)NC(=O)C(F)(F)F)[C@@H](CC1CC1)C(=O)N1C[C@@]2(C[C@H]1C#N)Oc1cc(F)c(F)cc1NC2=O ; CN(C(=O)[C@H](CC1CC1)NC(=O)C(F)(F)F)[C@@H](CC1CC1)C(=O)N1C[C@]2(C[C@H]1C#N)C(=O)Nc1ccnn12]

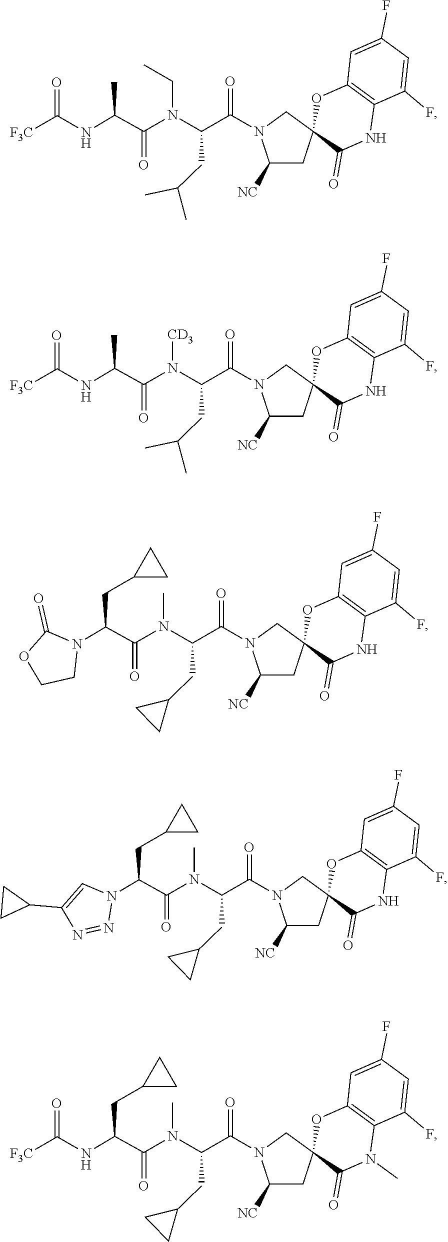 [SMILES: CCN(C(=O)[C@H](C)NC(=O)C(F)(F)F)[C@@H](CC(C)C)C(=O)N1C[C@@]2(C[C@H]1C#N)Oc1cc(F)cc(F)c1NC2=O ; [2H]C([2H])([2H])N(C(=O)[C@H](C)NC(=O)C(F)(F)F)[C@@H](CC(C)C)C(=O)N1C[C@@]2(C[C@H]1C#N)Oc1cc(F)cc(F)c1NC2=O ; CN(C(=O)[C@H](CC1CC1)N1CCOC1=O)[C@@H](CC1CC1)C(=O)N1C[C@@]2(C[C@H]1C#N)Oc1cc(F)cc(F)c1NC2=O ; CN(C(=O)[C@H](CC1CC1)n1cc(C2CC2)nn1)[C@@H](CC1CC1)C(=O)N1C[C@@]2(C[C@H]1C#N)Oc1cc(F)cc(F)c1NC2=O ; CN1C(=O)[C@]2(C[C@@H](C#N)N(C(=O)[C@H](CC3CC3)N(C)C(=O)[C@H](CC3CC3)NC(=O)C(F)(F)F)C2)Oc2cc(F)cc(F)c21]

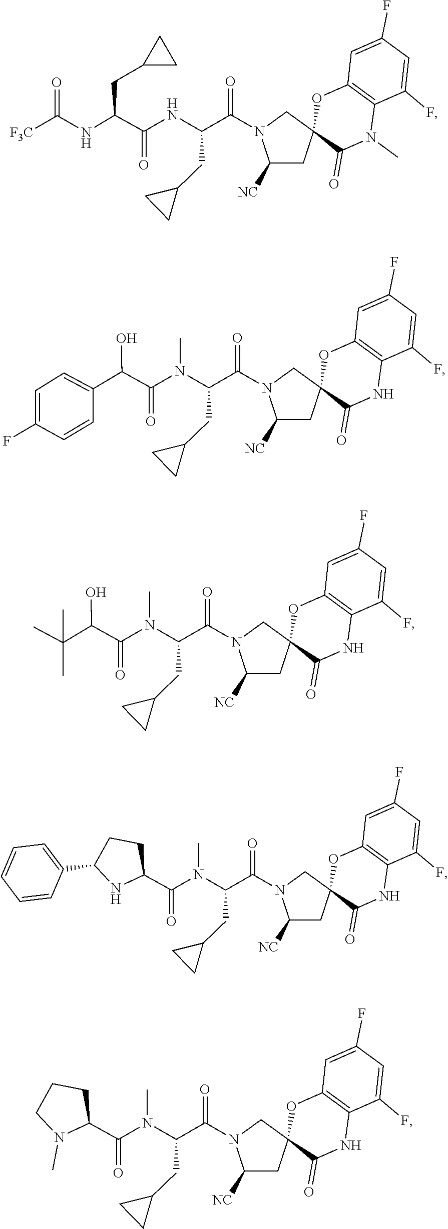 [SMILES: CN1C(=O)[C@]2(C[C@@H](C#N)N(C(=O)[C@H](CC3CC3)NC(=O)[C@H](CC3CC3)NC(=O)C(F)(F)F)C2)Oc2cc(F)cc(F)c21 ; CN(C(=O)C(O)c1ccc(F)cc1)[C@@H](CC1CC1)C(=O)N1C[C@@]2(C[C@H]1C#N)Oc1cc(F)cc(F)c1NC2=O ; CN(C(=O)C(O)C(C)(C)C)[C@@H](CC1CC1)C(=O)N1C[C@@]2(C[C@H]1C#N)Oc1cc(F)cc(F)c1NC2=O ; CN(C(=O)[C@@H]1CC[C@@H](c2ccccc2)N1)[C@@H](CC1CC1)C(=O)N1C[C@@]2(C[C@H]1C#N)Oc1cc(F)cc(F)c1NC2=O ; CN1CCC[C@H]1C(=O)N(C)[C@@H](CC1CC1)C(=O)N1C[C@@]2(C[C@H]1C#N)Oc1cc(F)cc(F)c1NC2=O]

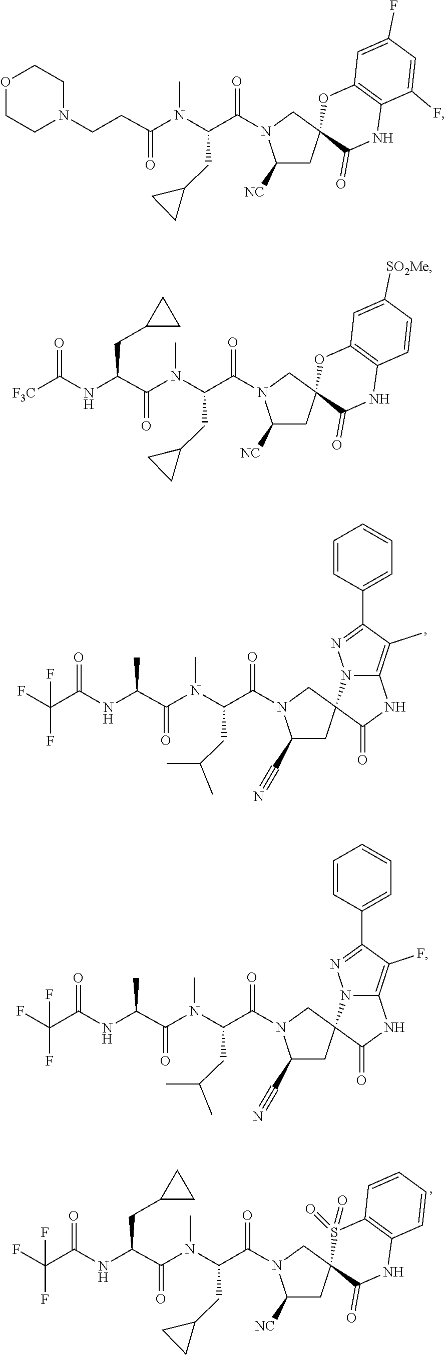 [SMILES: CN(C(=O)CCN1CCOCC1)[C@@H](CC1CC1)C(=O)N1C[C@@]2(C[C@H]1C#N)Oc1cc(F)cc(F)c1NC2=O ; CN(C(=O)[C@H](CC1CC1)NC(=O)C(F)(F)F)[C@@H](CC1CC1)C(=O)N1C[C@@]2(C[C@H]1C#N)Oc1cc(S(C)(=O)=O)ccc1NC2=O ; Cc1c(-c2ccccc2)nn2c1NC(=O)[C@]21C[C@@H](C#N)N(C(=O)[C@H](CC(C)C)N(C)C(=O)[C@H](C)NC(=O)C(F)(F)F)C1 ; CC(C)C[C@@H](C(=O)N1C[C@]2(C[C@H]1C#N)C(=O)Nc1c(F)c(-c3ccccc3)nn12)N(C)C(=O)[C@H](C)NC(=O)C(F)(F)F ; CN(C(=O)[C@H](CC1CC1)NC(=O)C(F)(F)F)[C@@H](CC1CC1)C(=O)N1C[C@]2(C[C@H]1C#N)C(=O)Nc1ccccc1S2(=O)=O]

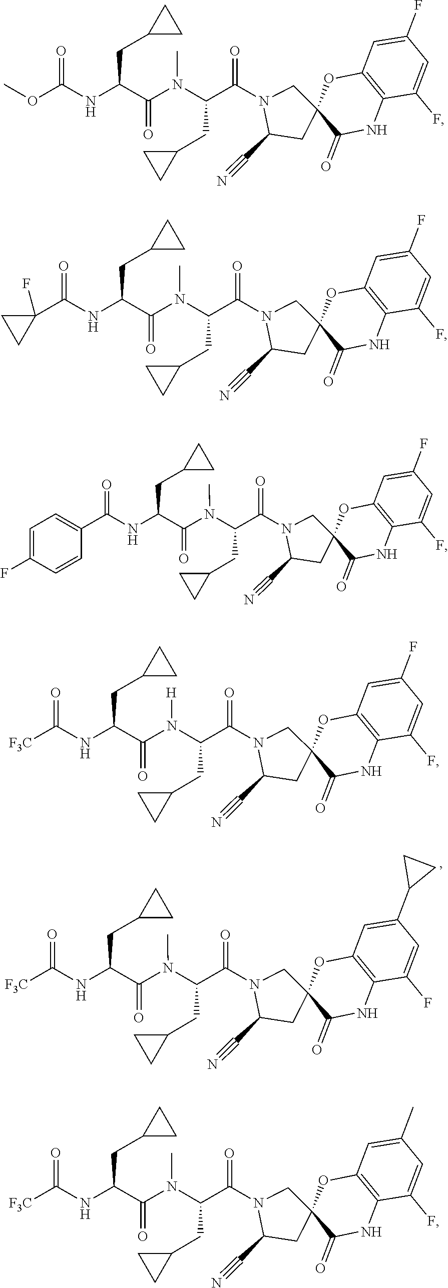

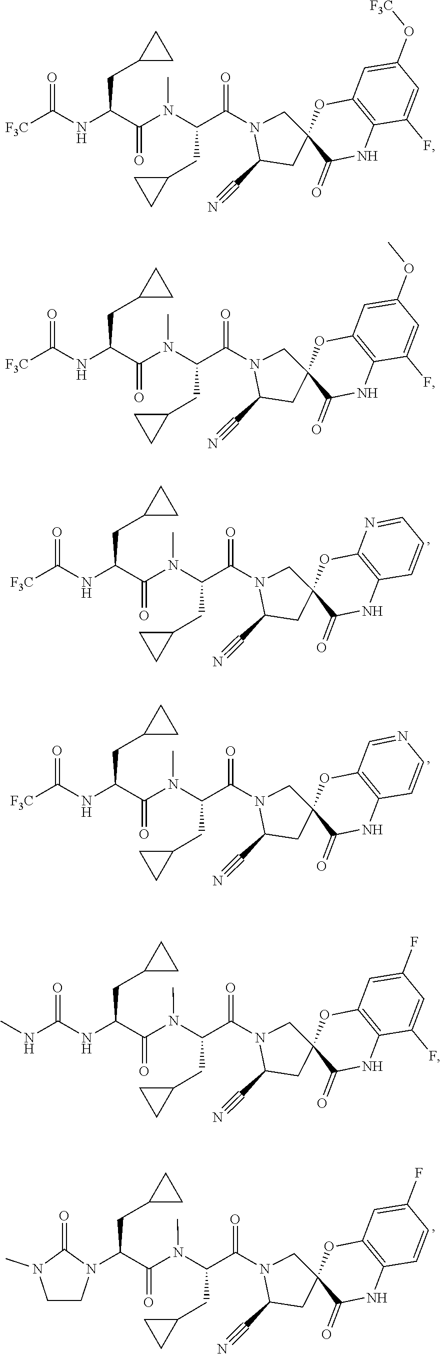

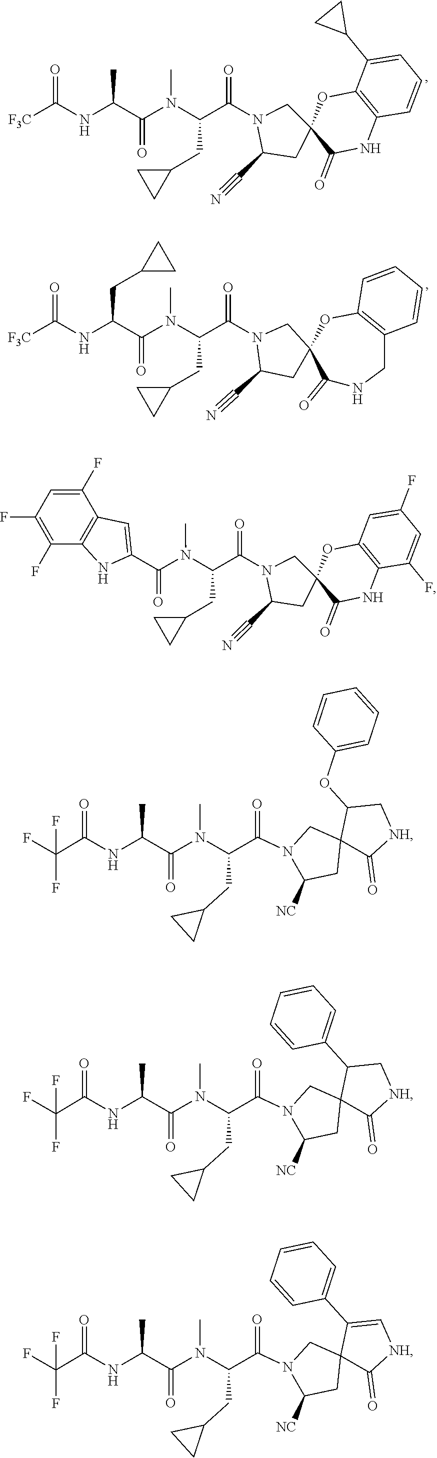

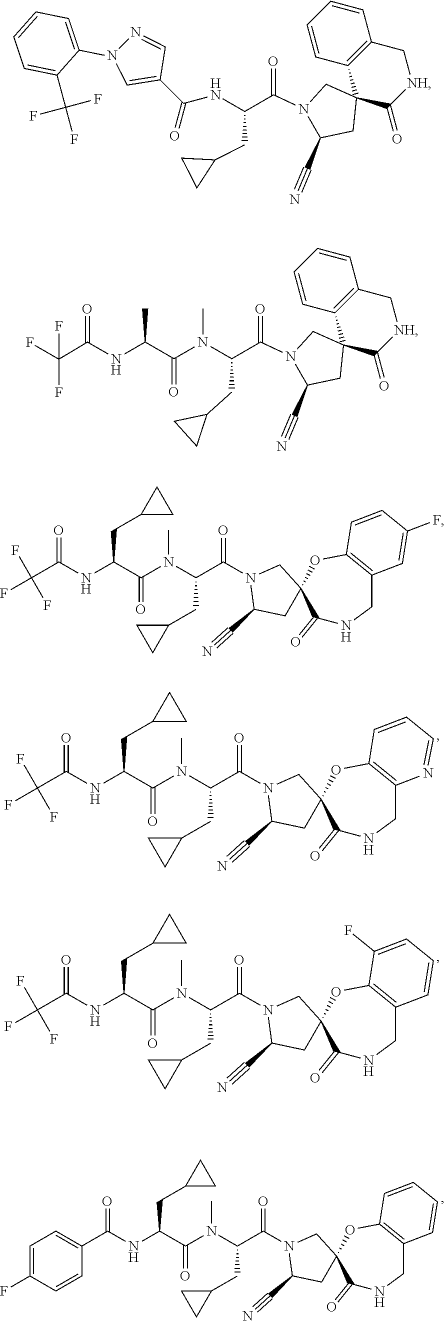

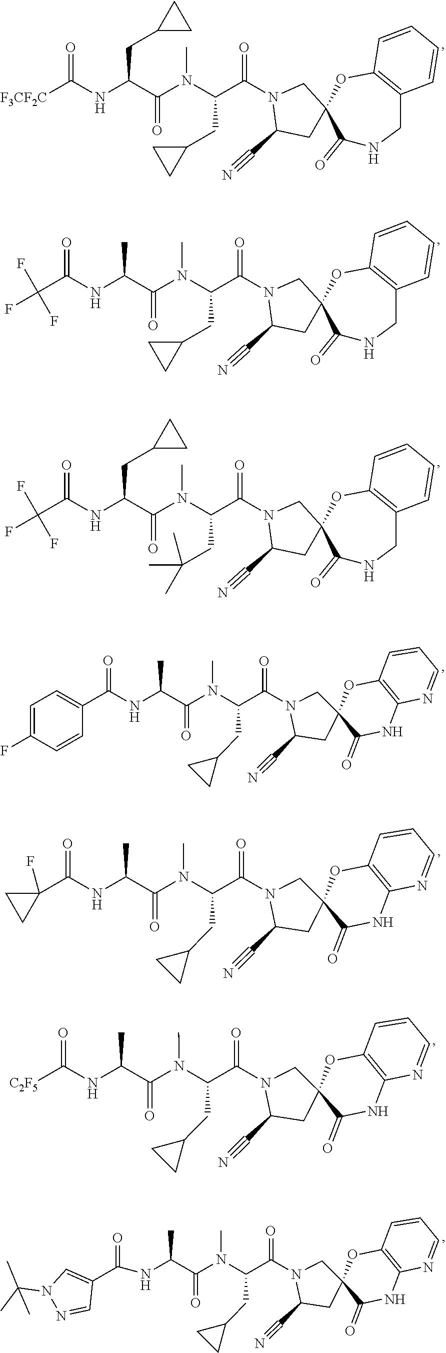

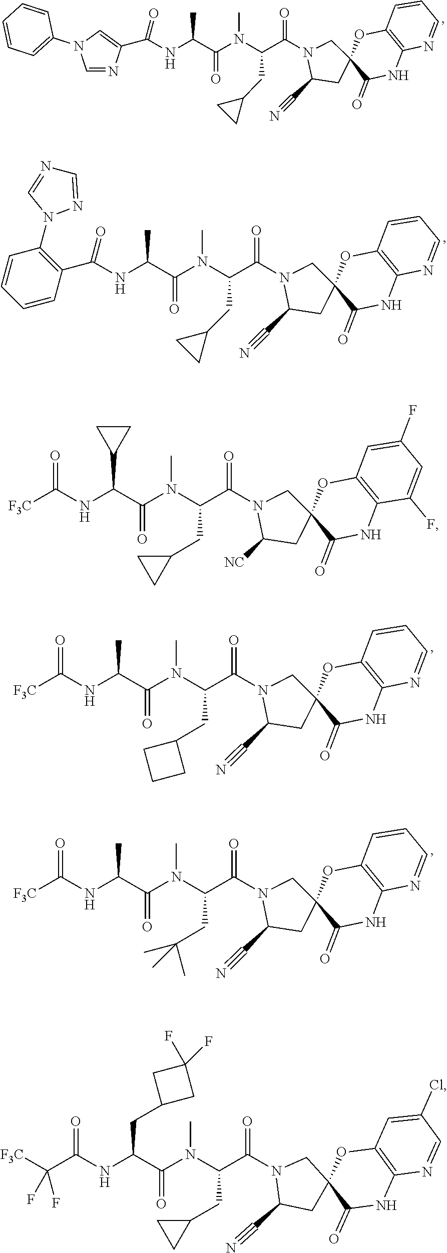

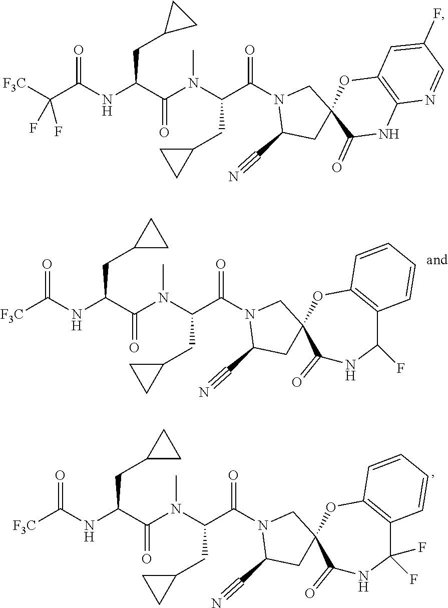

or a pharmaceutically acceptable salt of any of the foregoing.

### Synthesis

Compounds of Formula (I) along with those described herein may be prepared in various ways. General synthetic routes for preparing compounds of Formula (I) are shown and described herein along with some examples of starting materials used to synthesize compounds described herein. Additionally, for the purpose of the general synthetic routes, the structures depicted are appropriately protected, as known by one skilled in the art and the generic structures are meant to include these protecting groups. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

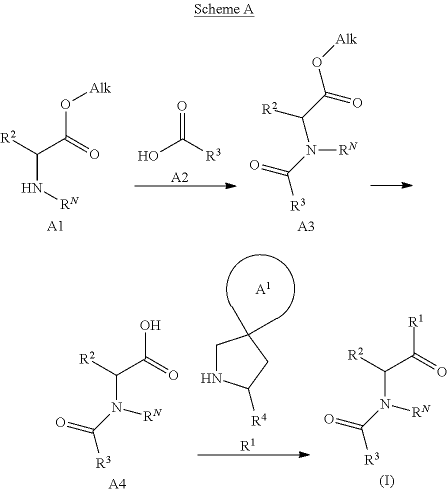

Scheme A describes the synthesis of compounds of Formula (I). An amino ester of Formula (A1) (wherein Alk represents alkyl) can react with an acid of Formula (A2), either by activating the carboxylic acid by converting it to an acid chloride, followed by reaction with the amino acid in the presence of a base, or by activation of the acid with a coupling reagent (such as HATU) followed by reaction with the amino ester in the presence of a base (such as DIPEA), resulting in a compound of Formula (A3). The ester functionality of Formula (A3) can be hydrolyzed, for example, under basic conditions if —OAlk is —OMe, using LiOH in MeOH, providing a compound of Formula (A4). Further coupling of the carboxylic acid of Formula (A4) with an amine of Formula (R¹) can provide a compound of Formula (I).

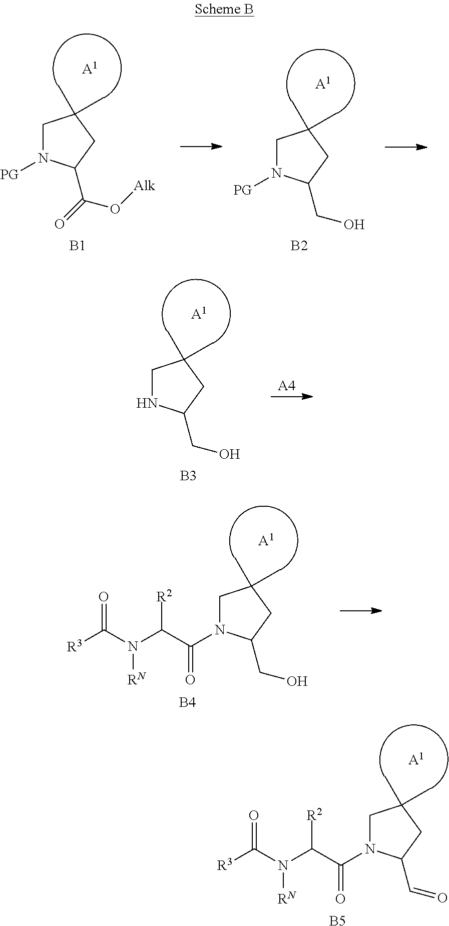

Scheme B depicts a general synthetic method to form an aldehyde of Formula (B5). An ester of Formula (B1) can be reduced to the alcohol of Formula (B2) using methods provided in the literature (e.g., LiBH₄). Removal of the protecting group (PG) can afford a compound of the Formula (B3) (e.g., HCl when PG is Boc). The amine of Formula (B3) can be coupled to a carboxylic acid of Formula (A4) using known coupling agents (e.g., HATU) to afford a compound of Formula (B4). Oxidation of the alcohol group of Formula (B4) can be accomplished using an oxidizing reagent described in the literature (such as IBX or Dess-Martin periodinane) and provide an aldehyde of Formula (B5).

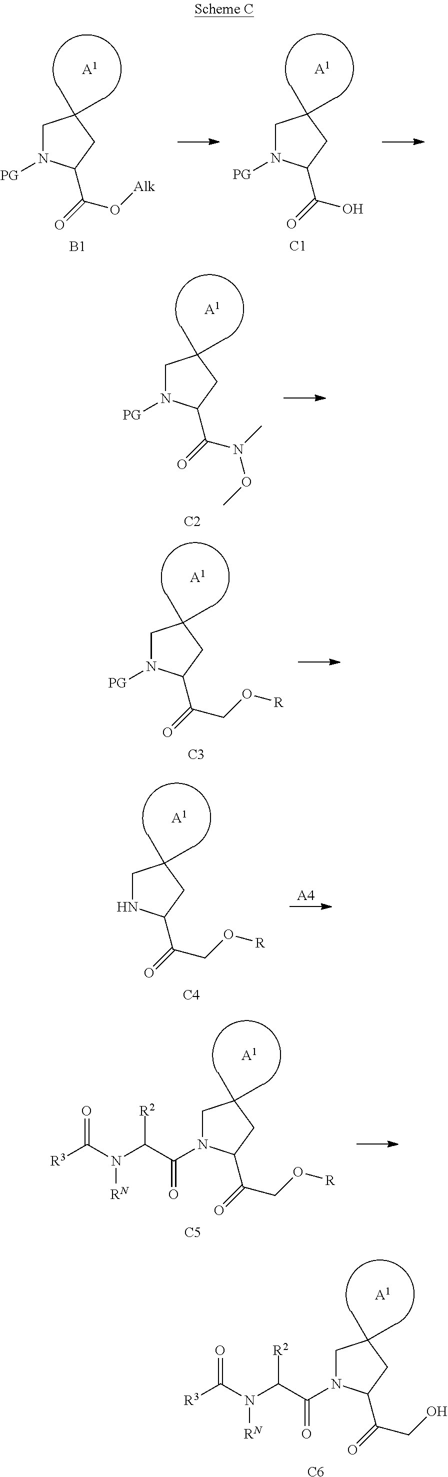

Scheme C depicts a general method to synthesize the hydroxyketones of Formula (C6). An ester of Formula (B1) can be hydrolyzed, for example with LiOH when Alk is methyl, to afford a carboxylic acid of Formula (C1). Conversion of compounds of Formula (C1) to an amide of Formula (C₂) can be accomplished by using a coupling agent (e.g., BOP, HATU, etc.) in the presence of N,O-dimethylhydroxyamine and a base (such as triethylamine) in an appropriate solvent (e.g., DMF). Addition of an organometallic reagent to the Weinreb amide of Formula (C2), followed by work-up, can result in a ketone of Formula (C3). An example, wherein R is benzyl, is the formation of an organometallic reagent by mixing Mg, HgCl₂ and benzylchloromethyl ether, followed by addition to a Weinreb amide of Formula (C2), followed by work-up with saturated ammonium chloride, (See Evans et al., Journal of the American Chemical Society (1988) 110(11):3560-3578 and Mendonca et al., Bioorganic & Medicinal Chemistry Letters (2002) 12(20):2887-2891) to afford an ether of the Formula (C3). Removal of the nitrogen protecting group (PG), (for example, using HCl or pTSA when PG is Boc) can afford a compound of Formula (C4). Subsequent coupling of a compound of Formula (C4) with a carboxylic acid of a compound of Formula (A4), using a coupling agent (e.g., TCFH, HBTU, etc.) can afford a compound of Formula (C5). The R group can be selectively removed (for example, by catalytic hydrogenation conditions (when R is benzyl (Bn), in Pd/C in a hydrogen atmosphere)) and provide a hydroxyketone of Formula (C₆).

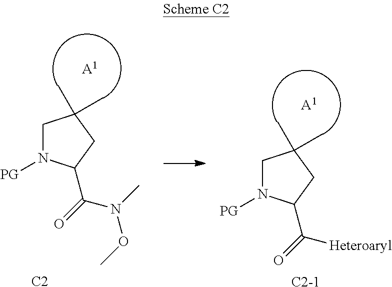

Another conversion method for R¹ described herein is shown in Scheme C₂. In Scheme C2, the Weinreb amide of Formula (C2) can be converted to a heterocyclic ketone of Formula (C2-1) using methods known to those skilled in the art (for example, Nahm et al., Tetrahedron Lett. (1981) 22(39), 3815-3818 and Balasubramaniam et al., Synthesis (2008) 23:3707-3738).

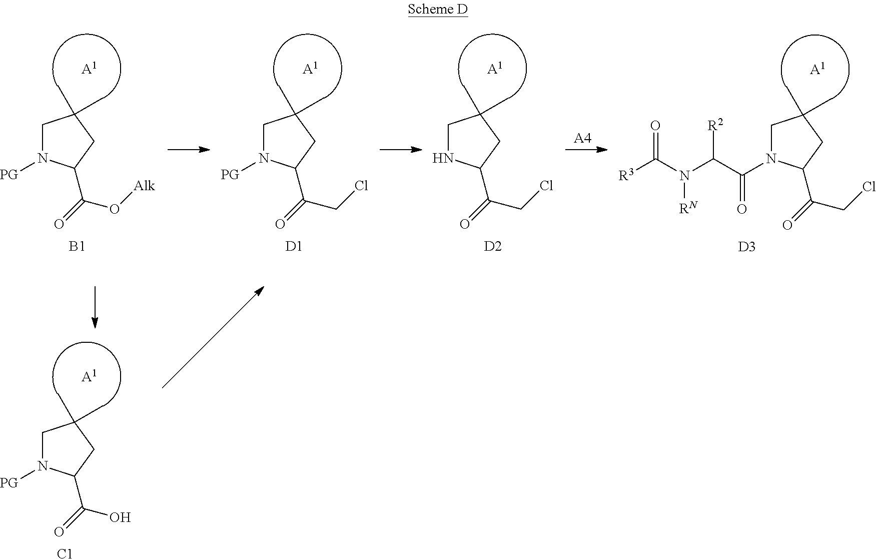

A general synthetic method to afford chloromethylketones of Formula (D3) is depicted in Scheme D. Compounds of Formula (B1) can be converted to a chloromethylketones of Formula (D1), for example, when Alk is methyl or ethyl, via methods known in the art (e.g., Pace et al., Advanced Synthesis & Catalysis (2013) 355(5):919-926 and Concellon et al., Journal of Organic Chemistry (2001) 66(25):8661-8665) using chloroiodomethane, or bromoiodomethane, and a strong base (such as LDA). An ester of Formula (B1) can be hydrolyzed to a compound of Formula (C1) and then converted to a chloromethylketone employing a variety of methods known in the art, including, but not limited to, the following: isopropyl chloroformate, 4-methylmorpholine, then diazomethane (See Sun et al., J. Med. Chem. 2006, 49(11):3153-3158). The protecting group of compounds of Formula (D1) can be cleaved to afford an amine of Formula (D2). Subsequent coupling of an amine of Formula (D2) with a carboxylic acid of Formula (A4) can be utilized using known coupling agents to provide chloromethylketones of Formula (D3).

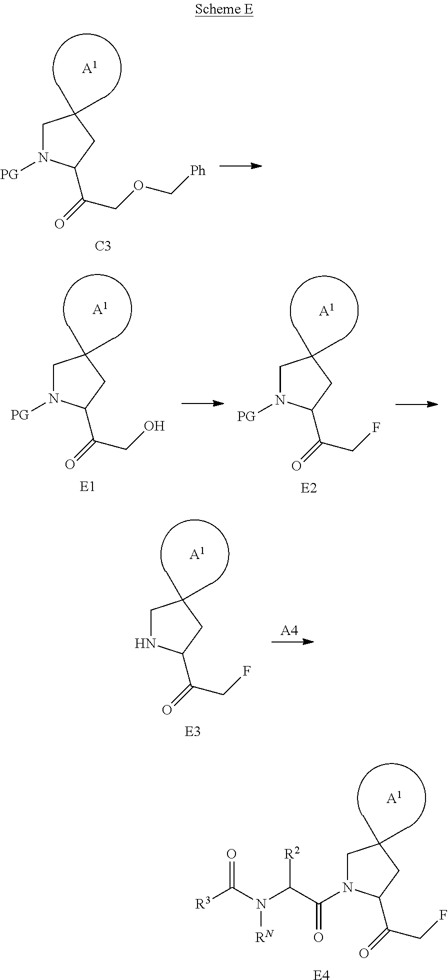

A general synthesis towards fluoromethylketone compounds of Formula (E4) is provided in Scheme E. The benzyl ether of Formula (C3) can be cleaved by Pd catalyzed hydrogenolysis (for example Pd/C in methanol) to afford a compound of Formula (E1). Subsequent conversion of an alcohol of Formula (E1) can be converted to a fluoromethylketone using fluorinating reagents that are known to those skilled in the art (e.g., a sulfonyl fluoride/HF-Et3N) to afford a fluoromethylketone of Formula (E2). Deprotection of the protecting group (PG) (for example, with HCl if PG is Boc) can afford compounds of Formula (E3). Compound of Formula (E3) can be then coupled with a carboxylic acid of Formula (A4) using a wide variety of commercially available coupling agents (such as TCFH in DMF with N-methyl imidazole) to afford a fluoromethylketone of Formula (E4). Alternatively, fluoromethylketone compounds of Formula (E2) can be obtained from a carboxylic acid of Formula (C1) via a modified Daikin-West reaction using fluoroacetic anhydride, triethylamine and DMAP in benzene (See Rasnick, D., Anal. Biochem. (1985) 149:461-465).

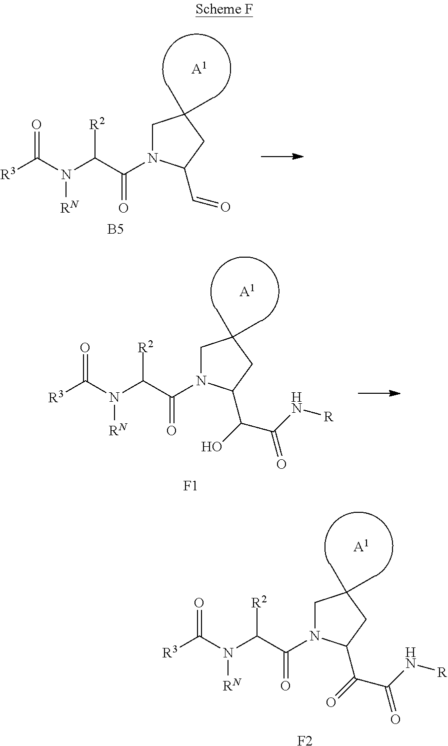

A general synthesis method towards a-ketoamides of Formula (F2) is described in Scheme F. The starting aldehyde of Formula (B5) can be treated with an isocyanide under conditions described in the literature to afford an alpha-hydroxyamide of Formula (F1). Subsequent oxidation of the alcohol group of a compound of Formula (F1) utilizing procedures know in the literature (such as Swern oxidation or Dess-Martin periodinane oxidation) can afford the a-ketoamide of Formula (F2).

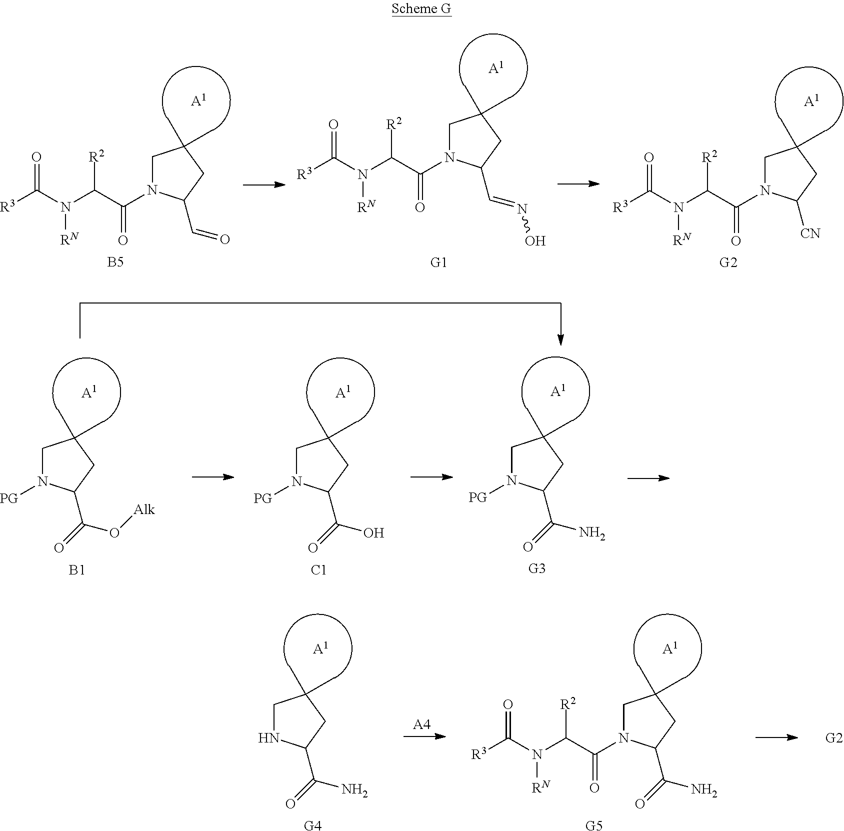

Scheme G illustrates a general method to synthesize nitrile compounds of Formula (G2). An aldehyde of Formula (B5) can be condensed with hydroxyamine HCl in an appropriate solvent (e.g., HMPA, DMSO) to afford oxime compound of Formula (G1). Subsequent reaction with Cu(OAc)₂ in HCl/CH₃CN, for example, provides a nitrile of Formula (G2). Alternatively, an aldehyde of Formula (B5) can react with 0-(4-(trifluoromethyl)benzoyl)hydroxylamine and L-(−)-camphorsulfonic acid (10%) in methanol to afford a nitrile of Formula (G2) (See An et al., Org. Lett. (2015) 17(20):5064-5067).

An additional method to prepare compounds of Formula (G2) is to start with a protected ester of Formula (B1) and then convert it directly to an amide of Formula (G3) by reaction with ammonia in THF or methanol in a sealed reactor. An ester of Formula (B1) can also be transformed into an amide of Formula (G3) via a two-step process where the ester of Formula (B1) is first hydrolyzed to a carboxylic acid (for example, using LiOH, water and THF when Alk is methyl) to a compound of Formula (C1). Subsequent transformation of the carboxylic acid of Formula (C1) to Formula (G3) can be carried out using a coupling agent (e.g., COMU, EDC) in the presence of ammonia, in an appropriate solvent (such as DMF or CH₃CN). Deprotection of the nitrogen protecting group of Formula (G3), for example, with HCl when PG is Boc, can generate the free amine which can be coupled with a compound of Formula (A4) (using a readily available amino acid coupling agent) to afford a compound of Formula (G5). Dehydration of the amide group of Formula (G5) using methods described in the literature, for example the Burgess reagent, or trifluoroacetic anhydride (TFAA) can provide the cyano compound of Formula (G2).

General methodology for the synthesis of amino acids of Formula (A2), or precursors that could be converted to an amino acid of Formula (A2) by one skilled in the art, are described in the literature, and include the following examples:

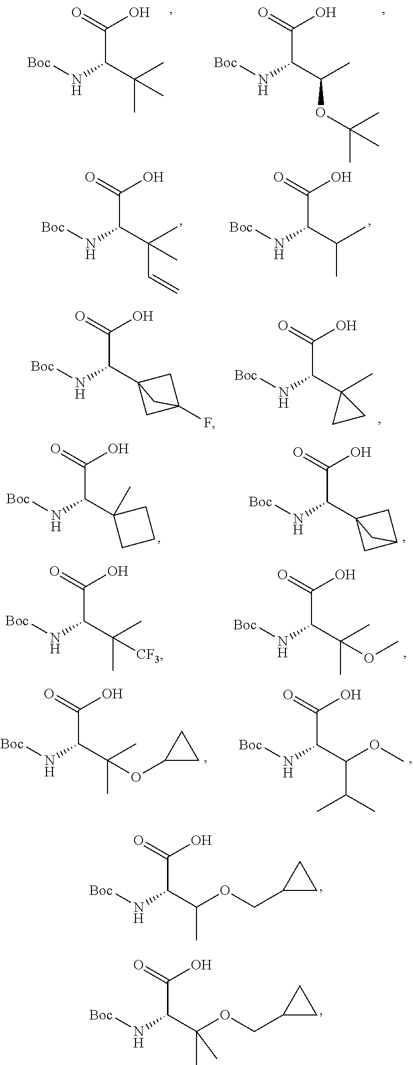

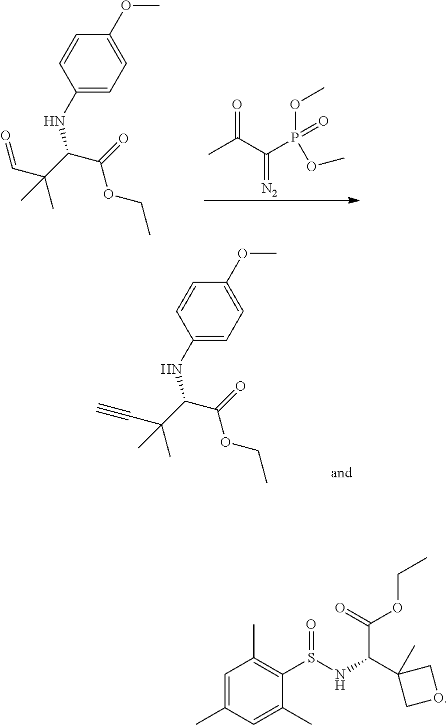

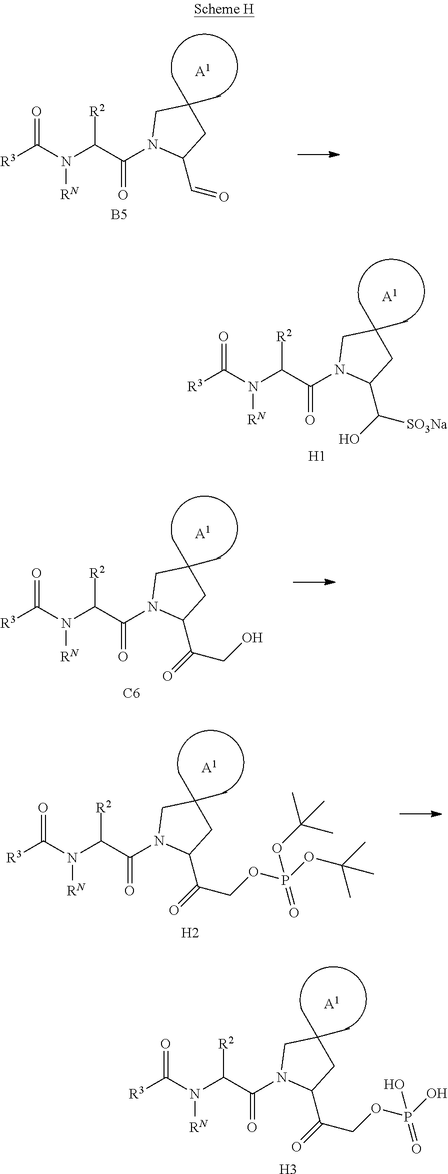

Compounds of Formula (I), or a pharmaceutically acceptable salt thereof, can include a prodrug-type and phosphate-containing moieties at R⁴. An example of a method is depicted in Scheme H. For example, an aldehyde of Formula (B5) can be transformed into the bisulfite adduct of Formula (H1), by treatment with NaHSO₃. A hydroxyketone of Formula (C6) can be transformed to the phosphate of Formula (H2), for example, by treatment with di-t-butyl N,N-dipropan-2-ylphosphoramidite and tetrazole followed by oxidation with H₂O₂. A compound of Formula (H2) can be deprotected (for example, by treatment with TFA) to provide a compound of Formula (H3).

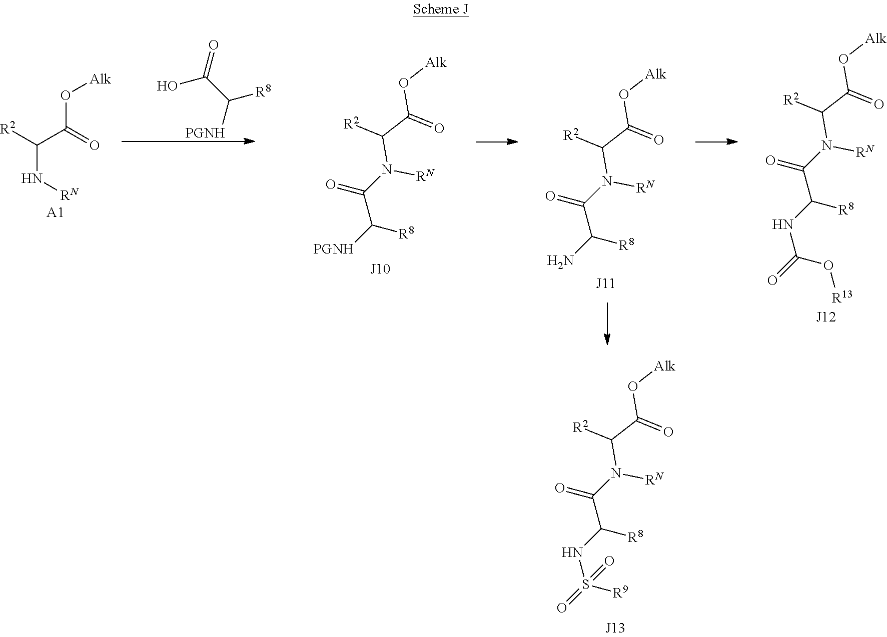

Scheme J describes the transformation of compounds of Formula (A1) to compounds of formulae (J12) and (J13). Compounds of Formula (A1) can be coupled to carboxylic acids using a variety of readily available peptide coupling agents (e.g., HATU, COMU, etc.). Compounds of Formula (J10) can be deprotected to provide an amine group. The amine group of compounds of Formula (J11) can be reacted with a chloroformate, in the presence of a base to form the carbamate compounds of Formula (J12). Alternatively, the amine group of Formula (J11) can be reacted with a sulfonyl chloride, in the presence of a base, to afford sulfonamides of Formula (J13).

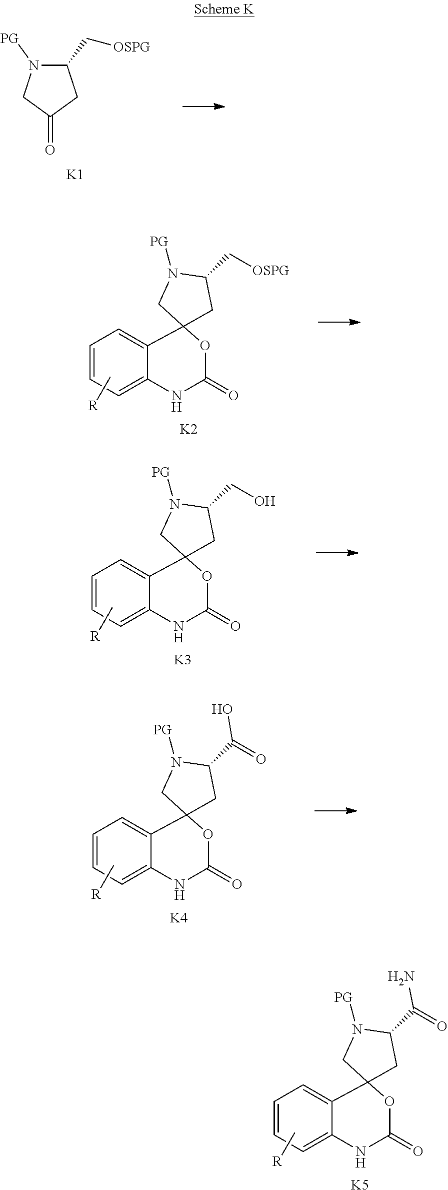

A general synthetic method to form compounds of the Formula (K5) are described in Scheme K. A protected ketone of Formula (K1) can be prepared according to methods known to those skilled in the art where PG is an amine protecting group such as Boc, and SPG is a silyl protecting group (e.g., TBDPS). An example is the synthesis of t-butyl (S)-2-(((t-butyldiphenylsilyl)oxy)methyl)-4-oxopyrrolidine-1-carboxylate described by Smits et al, J. Org. Chem. (2018) 83(9):5323-5330. Compounds of Formula (K1) can be transformed into spiro-benzoxazinones of Formula (K2) by the generation of a phenyl-lithium intermediate of Boc-aniline (using t-butyllithium at low temperature), and subsequent nucleophilic attack on the ketone of a compound of Formula (K1) to afford a tertiary alcohol. Subsequent ring closure can provide compounds of Formula (K2) (See Nicolaou et al, J. Am. Chem. Soc. (2009) 131(10):3690-3699) where R can be an optional substitution that is compatible with the synthesis. The silyl protecting group (SPG) of compounds of Formula (K2) can be deprotected under standard conditions (for example, using TBAF) to afford alcohol compounds of Formula (K3). Subsequent oxidation of alcohol of Formula (K3) can be done using various oxidating agents (such as e NaIO₄ and RuCl₃) to afford carboxylic acid compounds of Formula (K4). Compounds of Formula (K5) can be obtained by converting the acid group of Formula (K4) to a primary amide by methods known to those skilled in the art, for example, using ammonia with an amino acid coupling agent (such as EDC).

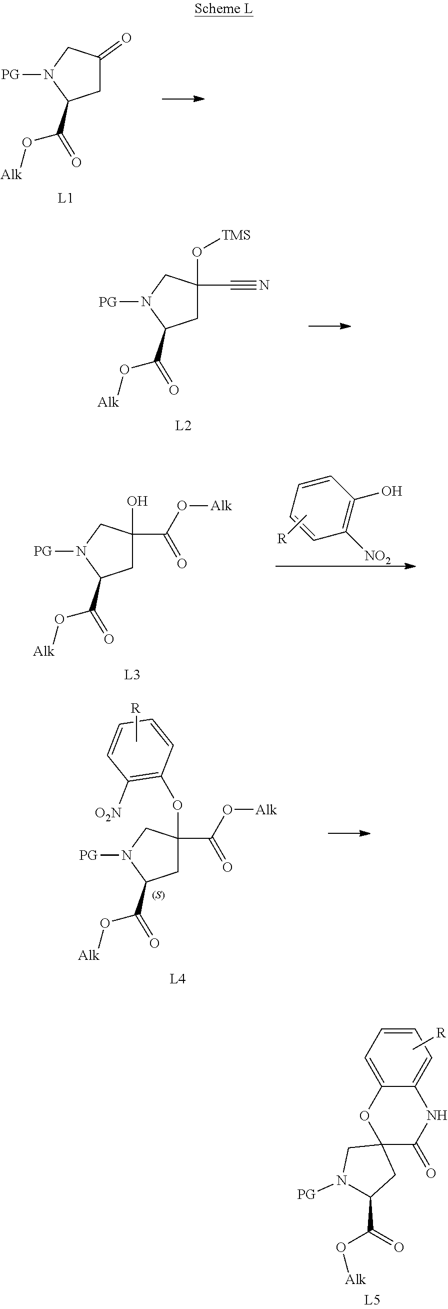

The synthesis of spiro-benzoxazinones of Formula L5, depicted in Scheme L, can begin with the conversion of a ketone of Formula (L1) to the protected cyanohydrin of Formula (L2) with the use of a silyl cyanide, such as TMSCN. Deprotection of the alcohol and alcoholysis of the CN group of compounds of Formula (L2) (for example, in HCl, CH₃OH) can lead to the formation of hydroxy-ester compounds of Formula (L3). Reaction with ortho-nitrophenols using typical Mitsunobu conditions can afford ether compounds of Formula (L4). Reduction of the nitro group of compounds of Formula (L4) can be accomplished under conditions known to those skilled in the art (e.g., Fe, NH₄Cl) in an alcoholic/aqueous solvent to afford the amine and concomitant ring closure to generate spirobenzoxazinones of Formula (L5).

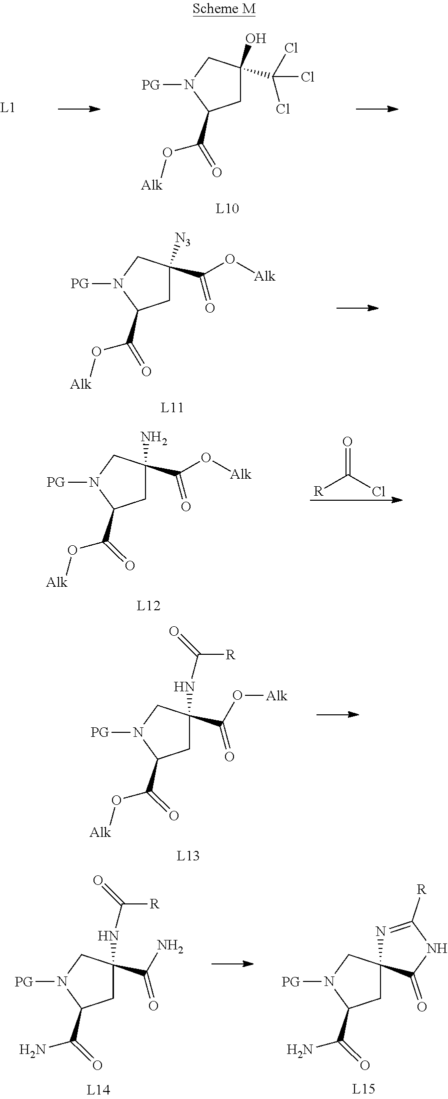

The generic synthesis towards spiroimidazolones is shown in Scheme M. Reaction of compounds of Formula (L1) under basic conditions with chloroform can lead to compounds of Formula (L10) (PG can be an amine protecting group). Compounds of Formula (L10) can be converted to azido esters of Formula (L11) under basic conditions with an azide salt (such as NaN₃). Reduction of the azide of compounds of Formula (L12) to the amino ester can be achieved by methods known in the literature (for example, by catalytic hydrogenation or reduction using PPh₃ and water). The reaction of compounds of Formula (L12) with an acid chloride, or alternatively, using standard carboxylic acid coupling conditions, can provide amides of Formula (L13). Conversion of the two ester groups of compounds of Formula (L13) to two primary amide groups, with the use of ammonia in methanol, applying heat, and preferably in a closed vessel, wherein Alk can be methyl or ethyl, can afford compounds of Formula (L14). Under basic conditions (e.g., potassium t-butoxide in methanol) with optional heating, compounds of Formula (L14) can be converted to spiroimidazolones of Formula (L15).

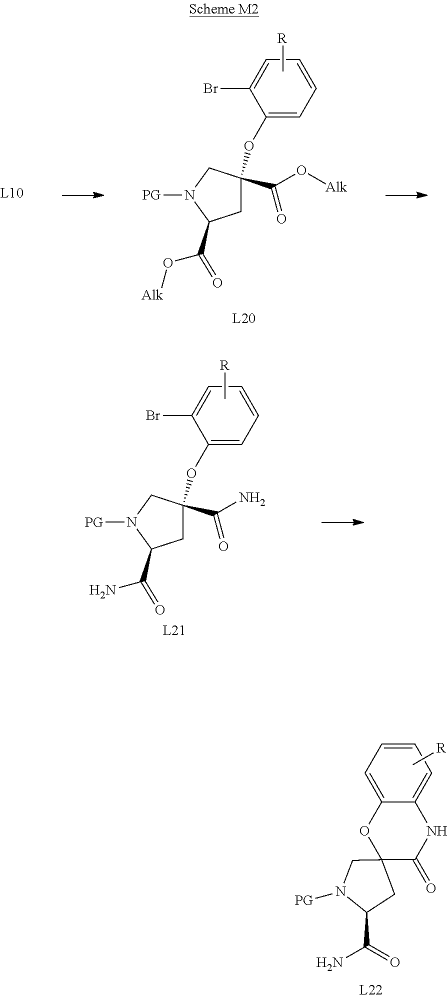

An alternative general synthetic method towards the compound series depicted in Scheme L is provided in Scheme M2. Compounds of Formula (L10) can be converted to compounds of Formula (L20) by reaction with an optionally substituted phenol in the presence of a base (such as NaOH) in an appropriate solvent (e.g., acetone). The two alkyl ester groups of Formula (L20) can be converted to the primary amides of Formula (L21) by heating with ammonia in a sealed vessel (facilitated when Alk is methyl or ethyl). Compounds of Formula (L21) can be reacted under copper catalysis conditions (e.g., CuI, DMEDA, Cs₂CO₃) to afford cyclization and provide compounds of Formula (L22).

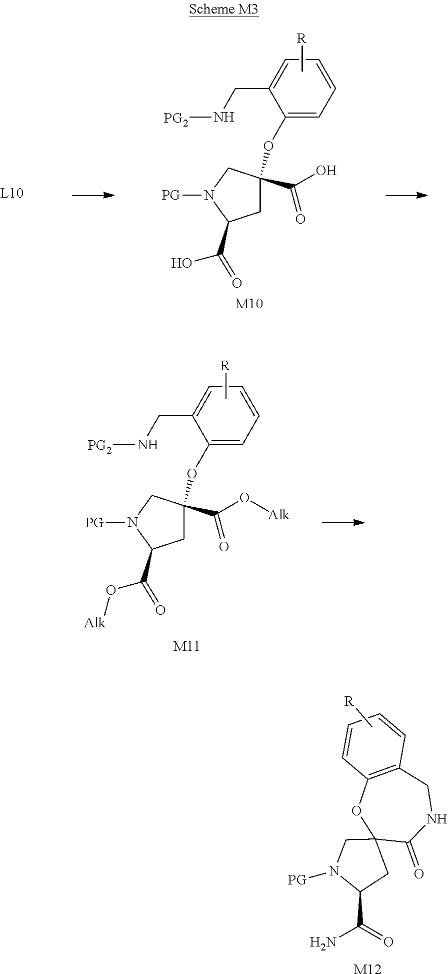

Similar to Scheme M2, scheme M3 depicts a synthetic pathway towards seven-membered spiro rings. Compounds of Formula (L10) can be converted to compounds of Formula (M10) by reaction with an optionally substituted, optionally protected, 2-(aminomethyl)phenol in the presence of a base (such as NaOH) in an appropriate solvent (e.g., acetone) with excess base resulting in the diacid of Formula (M10). Alkylation of the acid groups utilizing an alkyl halide and base (e.g., CH₃I, K₂CO₃, DMF) can result in a diester of Formula (M11). The removal of the protecting group (PG₂) either selectivity or unselectively over PG₁ can afford a primary amine that, upon heating with base (e.g. triethylamine), will cyclize to form spirocycles of Formula (M12).

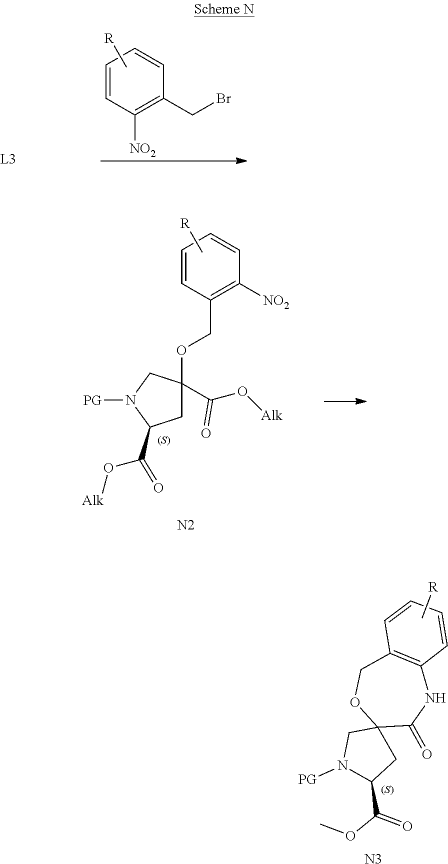

A general synthetic method to obtain compounds of Formula (N3) is provided in Scheme N. Compounds of Formula (L3) can be alkylated with a nitrobenzyl alkylbromide under standard alkylation conditions (for example, using K₂CO₃ in DMF) can afford ether compounds of Formula (N2). Conversion of compounds of Formula (N2) to cyclic compounds of Formula (N3) can be accomplished by reduction of the nitro group under conditions as described in the literature (e.g., Fe, NH₄Cl and water with optional heating).

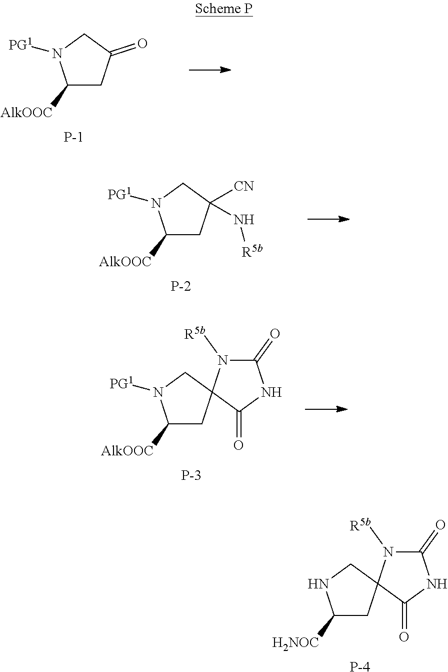

A spirohydantoin derivative of Formula (P-4) can be prepared as provided in Scheme P. A 4-oxoproline derivative of Formula (P-1) (with PG¹ representing a suitable nitrogen protecting group, for example, -Bn, prepared as described in Tetrahedron Asymmetry 1995, 1641) can be treated with potassium cyanide and an amine to afford an amino nitrile derivative of Formula (P-2). Compounds of Formula (P-2) can be converted to a spirohydantoin of Formula (P-3) using potassium cyanide and ammonium carbonate in acidic conditions. After protecting group removal (for example, by palladium-catalyzed hydrogenation when PG¹ is Bn) and ester aminolysis (wherein Alk is methyl or ethyl), an amine of Formula (P-4) can be obtained. The spirohydantoin compounds can be resolved using various techniques (for example, purification by chromatography).

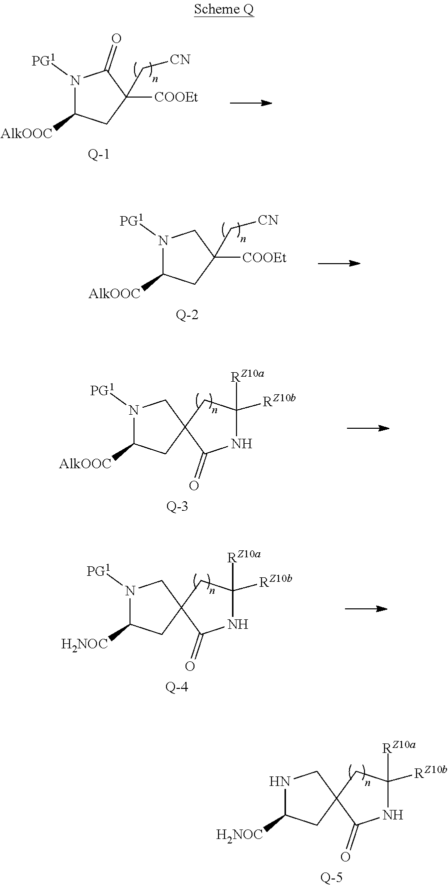

A spirolactam derivative of Formula (Q-5) can be prepared as provided in Scheme Q. Pyrrolidinone of Formula (Q-1) when n is 1, Alk is Et and PG¹ is Boc can be prepared as described in Cowley et al., Org. Biomol. Chem. (2011) 9:7042-7056. Pyrrolidinone of Formula (Q-1) when n is 2, Alk is Et and PG¹ is Boc can be prepared by Michael reaction with acrylonitrile and 1-(t-butyl) 2,4-diethyl (2S)-5-oxopyrrolidine-1,2,4-tricarboxylate synthesized as described in Cowley et al., Org. Biomol. Chem. (2011) 9:7042-7056. Selective reduction of a pyrrolidinone of Formula (Q-1) (with PG¹ representing a suitable nitrogen protecting group, for example, -Boc) using lithium triethylborohydride followed by further reduction of the hemiaminal intermediate with triethysilane and boron trifluoride etherate (Dorta et al., Tetrahedron Lett. (1994) 35(13):2053-2056) can provide a pyrrolidine of Formula (Q-2). Nitrile reduction (for example, with CoCl₂ and NaBH₄), and subsequent cyclisation in-situ can provide a lactam of Formula (Q-3). Alternatively, a nitrile of Formula (Q-2) can be converted via a Kulinkovich-Szymoniak reaction into a cyclopropyl amine, which can react with the ethyl ester in-situ to afford a lactam of Formula (Q-3). A primary amide of Formula (Q-4) can be prepared by aminolysis of an ester of Formula (Q-3), when Alk is methyl or ethyl, or by ester hydrolysis in basic conditions followed by the reaction with ammonia under typical amide coupling conditions. Protecting group removal (for example, when PG¹ is Boc, by treatment with HCl) can provide an amine of Formula (Q-5). The spirolactam intermediates can be resolved using various techniques (such as purification by chromatography).

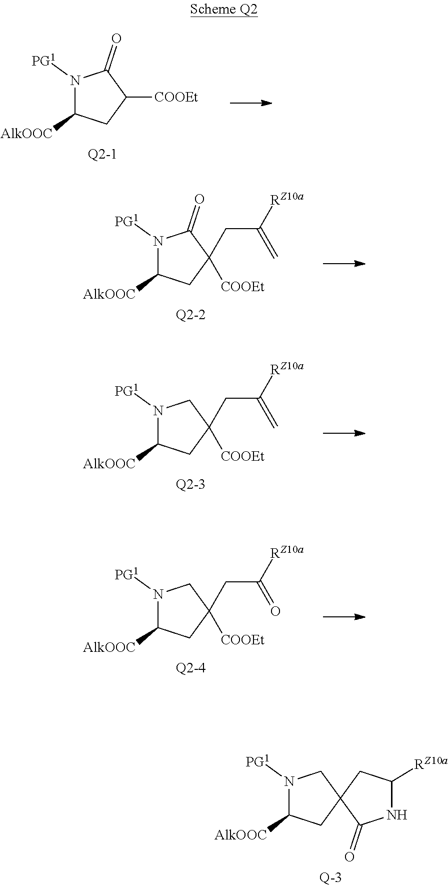

Alternatively, lactams of Formula (Q-3) when n is 1, R^{Z10b} is H and PG¹ is Boc can be prepared as provided in Scheme Q2. Alkylation of a pyrrolidinone of Formula (Q2-1) (synthesized as described in Cowley et al., Org. Biomol. Chem. (2011) 9:7042-7056) with an allyl halide in presence of a base (such as sodium hydride) can provide allyl of Formula (Q2-2). A pyrrolidine of Formula (Q2-3) can be prepared by reduction using lithium triethylborohydride followed by treatment with triethysilane and boron trifluoride etherate as described for the pyrrolidine of Formula (Q-2). A ketone of Formula (Q2-4) can be prepared by oxidative cleavage using, for example, osmium tetroxide and sodium periodate. Reductive amination with ammonium acetate and a reducing agent (such as sodium cyanoborohydride followed by cyclisation in-situ) can provide a lactam of Formula (Q-3).

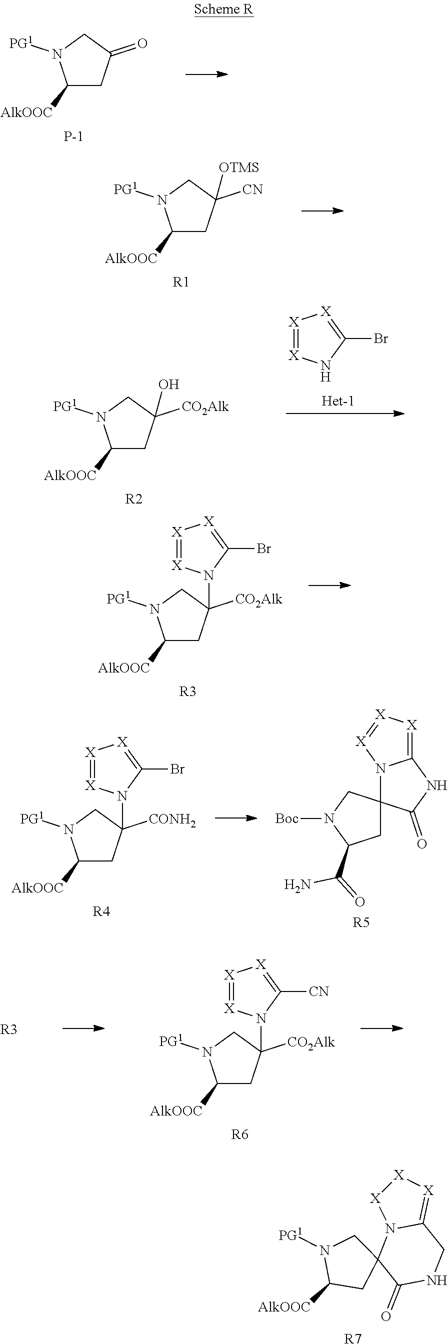

A general synthesis of spiro heterocycles is provided in Scheme R. A ketone of Formula (P-1) can be transformed into a cyanohydrin using methods known to those skilled in the art to afford a compound of Formula (R¹). Alcoholysis of a cyanohydrins of Formula (R1) using HCl in methanol with optional heating to afford a hydroxyester Formula (R²). During the synthesis of a compound of Formula (R2), it is possible to cleave acid labile protecting groups, such as Boc. The nitrogen can be protected again ((for example, using Boc by introduction of Boc-anhydride in an organic solvent and base) to afford a compound of Formula (R2). Compounds of Formula (R2) can react with various heterocycles (Het-1, where each X can be CR or N) where the nitrogen of the heterocyclyl can displace the OH under typical Mitsunobu reaction conditions to afford a compound of Formula (R3). Bis-amides of Formula (R4) can be formed by reacting Formula (R3) with concentrated ammonia (such as dissolved in an organic solvent) in a pressure reactor or thick-walled sealed tube. Bis-primary amides of Formula (R4) can undergo a Cu-catalyzed (for example, CuI, DMEDA, Cs₂CO₃, THF, 70° C.) or Pd-catalyzed ring closure to form a compound of Formula (R5).

Alternatively, spirocycles may be formed by taking advantage of the reactive Br. For example, compounds of Formula (R3) can react with Zn(CN)₂, Zn, a ligand and a Pd catalyst (e.g., Pd₂(dba)₃) at elevated temperatures to obtain a cyano-heterocyclyl of Formula (R6). Hydrogenation of Formula (R6) can afford a primary amine that can undergo ring closure by reacting with the ester group to form a six-membered ring, as depicted by a compound of Formula (R7).

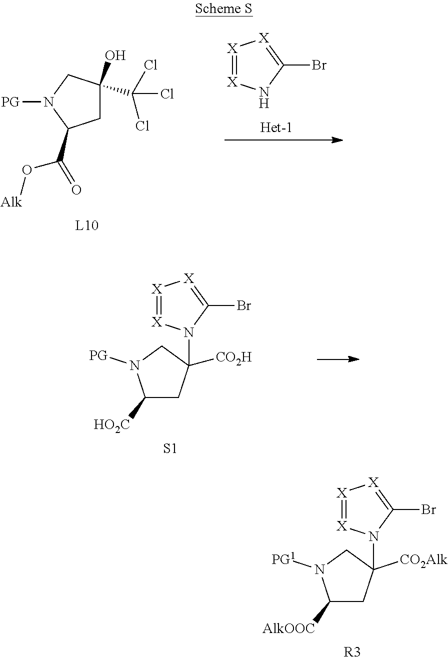

Another approach to the heterocycles provided in Scheme R is depicted in Scheme S. Compounds of Formula (L10) can be reacted with a heterocycle (Het-1, where X can be CR or N) in the presence of a base (e.g., NaOH) and an organic solvent (e.g., ethanol, acetone and/or THF) to afford a compound of Formula (S1). A diacid of Formula (S1) can be alkylated to a diester of Formula (R3) using an alkyl halide and either an organic or mineral base (e.g., K₂CO₃) in an organic solvent.

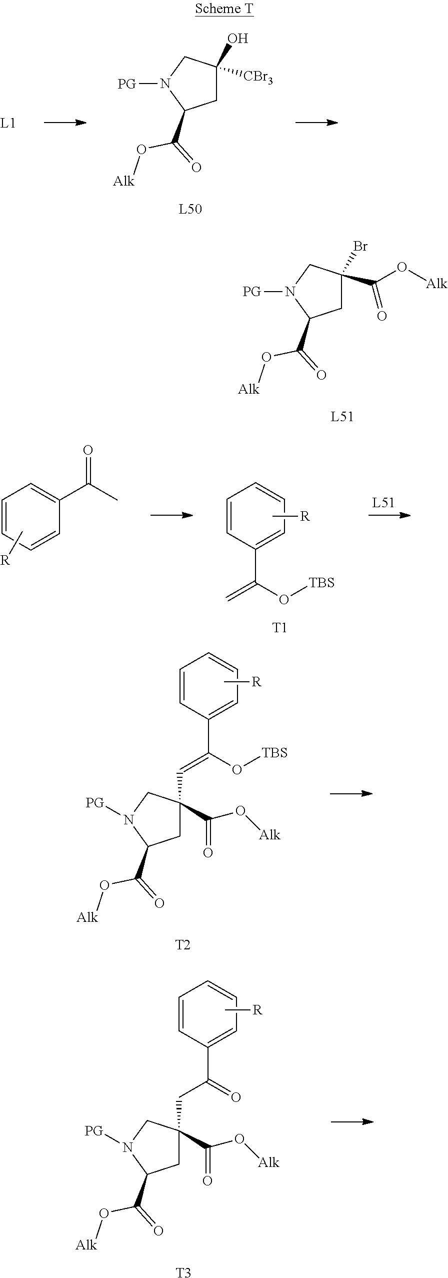

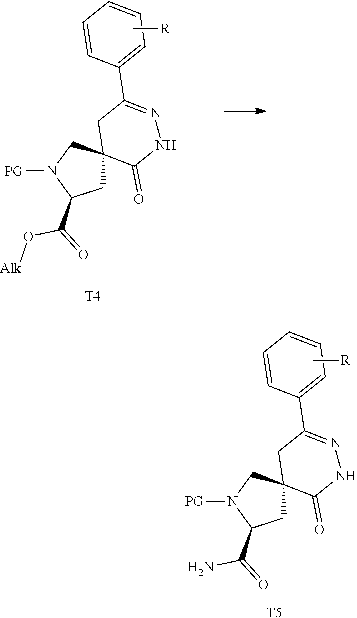

A general synthesis towards spiropyridazinones is provided in Scheme T. Acetophenones can be converted to an enol ether of Formula (T1) using methods know to those skilled in the art (for example, TBSCl, NaI, triethylamine in CH₂Cl₂). In parallel, a compound of Formula (L50) can be prepared using a similar procedure for the formation of a compound of Formula (L10) with the exception that bromoform can be used instead of chloroform. Compounds of Formula (L50) can be transformed into compounds of Formula (L51) using a base in an alcoholic solvent (such as DBU in methanol). Compounds of Formula (L51) can react with an enol ether of Formula (T1) using a copper catalyst and base to form compounds of Formula (T2). Deprotection of the enol ether, for example, with TBAF in THF, can afford a ketone of Formula (T3), which can be reacted with hydrazine (in an organic solvent, with optional heating) to afford a compound of Formula (T4). The ester of Formula (T4) can be converted to a primary amide of Formula (T5) via addition of concentrated ammonia in an organic solvent, optionally heated under pressure.

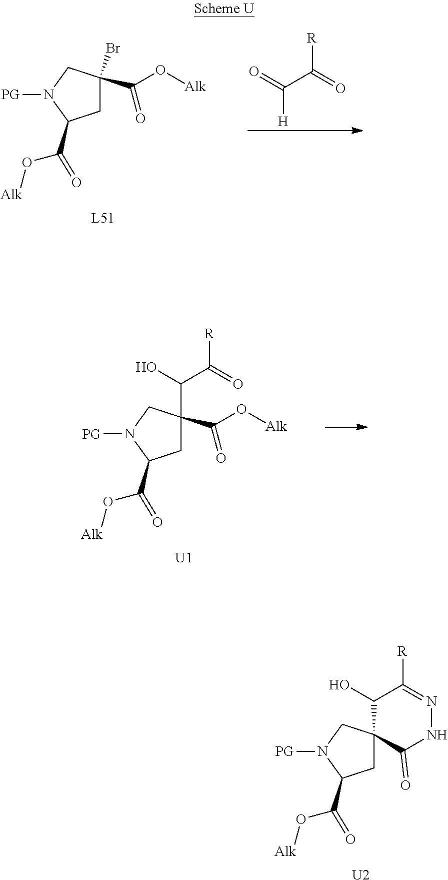

An alternative synthesis to spiropyridazinones is provided in Scheme U where R can be an alkyl or an aryl. Compounds of Formula (L51) can react with glyoxals in a zinc mediated reaction to form a hydroxy ketone of Formula (U1). Reaction of compounds of Formula (U1) with hydrazine can form the ring closed compounds of Formula (U2). The resulting alcohol of Formula (U2) can be maintained as-is or optionally removed (for example, by silane reduction) or oxidized to a ketone, which can be further functionalized.

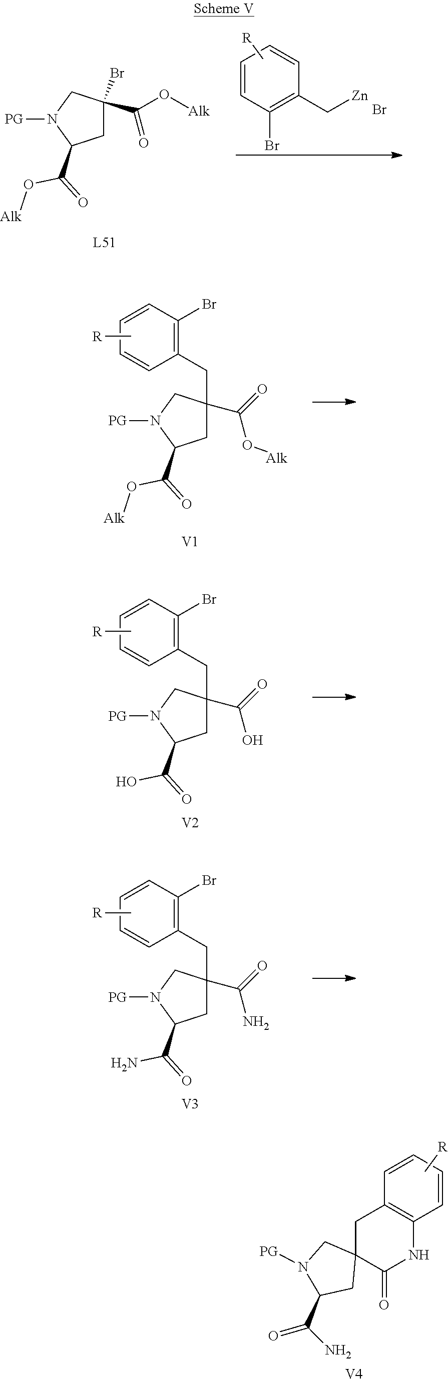

A general synthesis to prepare spirolactams is shown in Scheme V. Starting bromides of Formula (L51) can be reacted with a (2-bromobenzyl) zinc bromide via a cobalt catalyst in an organic solvent to afford a compound of Formula (V1). Subsequent conversion of a compound of Formula (V1) to the bis-acid of Formula (V2) can be accomplished via basic hydrolysis. A bis-amide of Formula (V3) can be formed by reacting the bis acid of Formula (V2) with ammonia and a coupling catalyst. Ring closure via copper catalysis can afford a compound of Formula (V4).

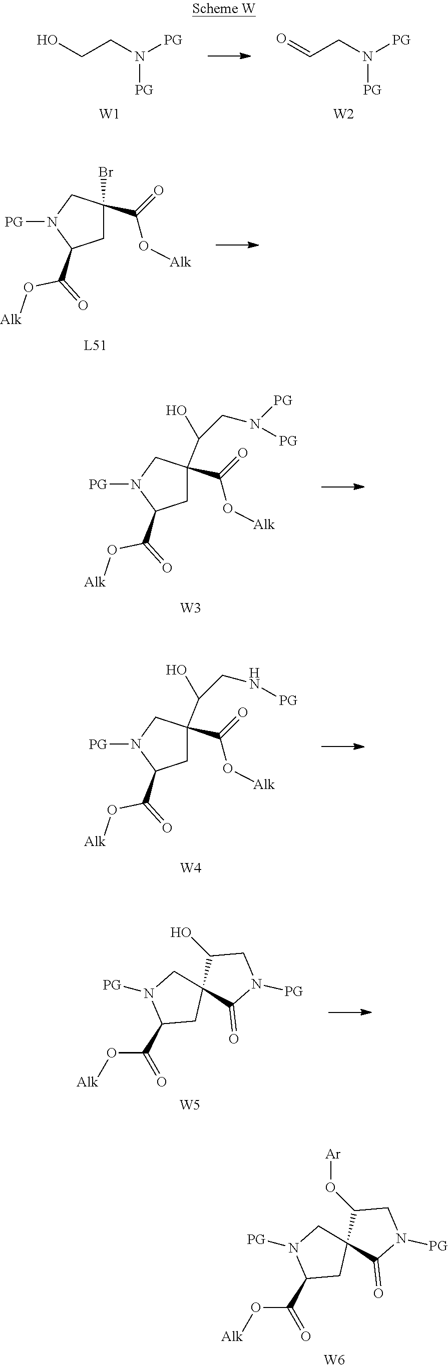

Scheme W provides a general synthesis of aryloxy substituted spirolactams. A protected amino alcohol of Formula (W1) can be oxidized to an aldehyde of Formula (W2) using a variety of conditions known to those skilled in the art. A compound of Formula (W2) can react with a compound of Formula (L51) and zinc to afford a compound of Formula (W3). One of the protecting groups of Formula (W3) can be selectively removed using a method known to those skilled in the art to afford a compound of Formula (W4). Heating a compound of Formula (W4) in the presence of base (for example, K₂CO₃ in methanol) can provide ring-closed compound of Formula (W5). Aryl ethers of Formula (W6) can be formed under typical Mitsunobu conditions by reacting a compound of Formula (W5) with an optionally substituted phenol. Alternatively, the alcohol group of compounds of Formula (W5) can be alkylated with an alkyl halide in the presence of base.

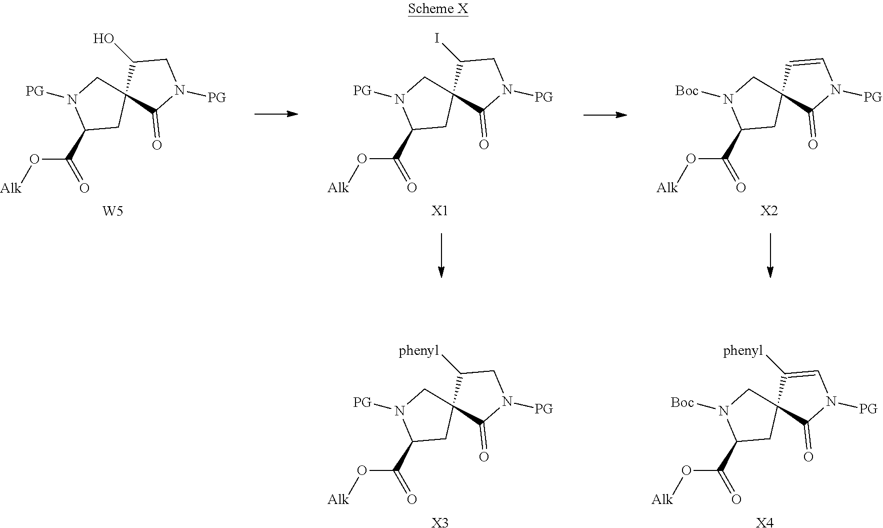

Scheme X provides a general synthesis of phenyl substituted spirolactams. The alcohol of a compound of Formula (W5) can be exchanged for a halogen (for example, iodine) via the Appel reaction using iodine and triphenylphosphine. An alkyl iodide of Formula (X1) can undergo a Suzuki reaction with a phenyl borane or phenyl boronic acid, using procedures described known to those skilled in the art to afford a compound of Formula (X3). Alternatively, the halogen of a compound of Formula (X1) can be eliminated to form a double bond compound of Formula (X2). Compounds of Formula (X2) can undergo a Heck-type coupling reaction using methods known to those skilled in the art to afford a compound of Formula (X4).

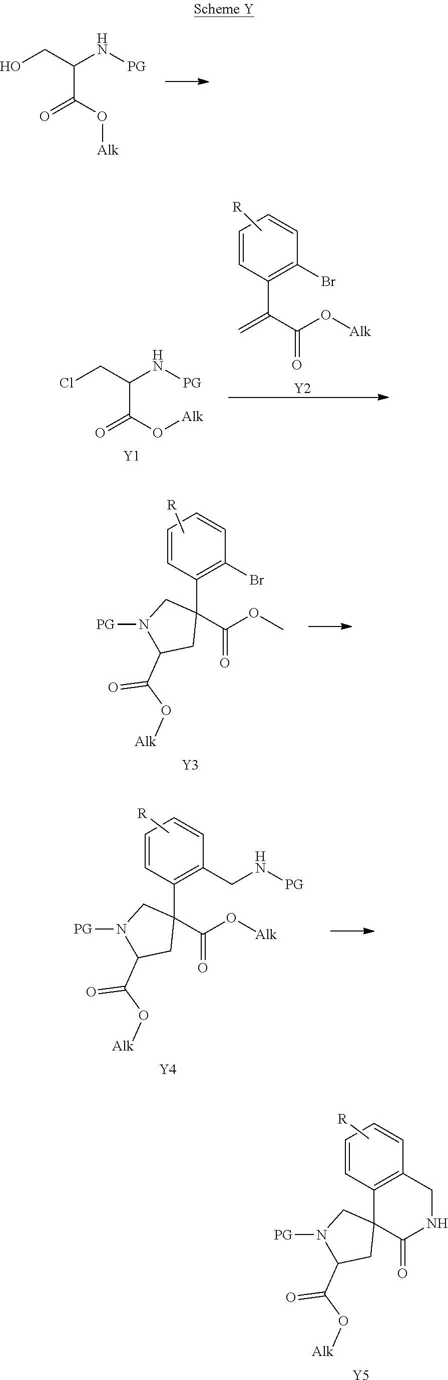

Depicted in Scheme Y provides a synthesis towards spirolactams of Formula (Y5). Commercially available protected serine esters can be converted to an alkyl chloride of Formula (Y1) using the Appel reaction (PPh₃, CCl₄). A compound of Formula (Y1) can undergo a cycloaddition with an aryl acrylate of Formula (Y2) to afford a cyclic compound of Formula (Y3). Subsequent formation of a compound of Formula (Y4) can be accomplished via a Suzuki coupling of a vinyl potassium trifluoroborate with the aryl bromide of Formula (Y3). Alternatively, formation of a compound of Formula (Y4) can be accomplished by reaction of the aryl bromide of Formula (Y3) with Zn(CN)₂ with the aid of a Pd catalyst, followed by reduction of the CN group to afford a compound of Formula (Y4) where PG is hydrogen. Compounds of Formula (Y4) can be cyclized by deprotection of the amine, followed by heating, or alternatively, deprotection of the amine and the ester using a coupling agent to afford a spirocyclic lactam of Formula (Y5).

### Pharmaceutical Compositions

Some embodiments described herein relate to a pharmaceutical composition, that can include an effective amount of a compound described herein (e.g., a compound, or a pharmaceutically acceptable salt thereof, as described herein) and a pharmaceutically acceptable carrier, excipient or combination thereof. A pharmaceutical composition described herein is suitable for human and/or veterinary applications.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Proper formulation is dependent upon the route of administration chosen. Techniques for formulation and administration of the compounds described herein are known to those skilled in the art. Multiple techniques of administering a compound exist in the art including, but not limited to, oral, rectal, topical, aerosol, injection, inhalation and parenteral delivery, including intramuscular, subcutaneous, intravenous, intramedullary injections, intrathecal, direct intraventricular, intraperitoneal, intranasal and intraocular injections. Pharmaceutical compositions will generally be tailored to the specific intended route of administration.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes may be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

### Methods of Use

Some embodiments described herein relate to a method of treating a coronavirus infection that can include administering to a subject identified as suffering from the coronavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a coronavirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a coronavirus infection.

Some embodiments disclosed herein relate to a method of treating a coronavirus infection that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a coronavirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a coronavirus infection.

Some embodiments disclosed herein relate to a method of inhibiting replication of a coronavirus that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a coronavirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a coronavirus.

In some embodiments, the coronavirus can be an α-coronavirus or a β-coronavirus. A compound described herein may be effective against one or more variants of a coronavirus. Examples of variants include, but are not limited, to alpha-variant (B.1.1.7), beta-variant (B.1.351), gamma variant (P.1) and delta-variant (B.1.617.2). In some embodiments, the coronavirus can be selected from CoV 229E, CoV NL63, CoV OC43, CoV HKU1, Middle East Respiratory Syndrome (MERS)-CoV, Severe Acute Respiratory Syndrome (SARS)-CoV, and SARS-CoV-2.

Some embodiments described herein relate to a method of treating a picornavirus infection that can include administering to a subject identified as suffering from the picornavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a picornavirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a picornavirus infection.

Some embodiments disclosed herein relate to a method of treating a picornavirus infection that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a picornavirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a picornavirus infection.

Some embodiments disclosed herein relate to a method of inhibiting replication of a picornavirus that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a picornavirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a picornavirus.

In some embodiments, the picornavirus can be a rhinovirus, including rhinovirus A, B and/or C. In some embodiments, a compound described herein, including a compound of Formula (I), or a pharmaceutically acceptable salt thereof, can be used to treat one or serotypes of a rhinovirus.

Some embodiments described herein relate to a method of treating a norovirus infection that can include administering to a subject identified as suffering from the norovirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a norovirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a norovirus infection.

Some embodiments disclosed herein relate to a method of treating a norovirus infection that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a norovirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a norovirus infection.

Some embodiments disclosed herein relate to a method of inhibiting replication of a norovirus that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a norovirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a norovirus.

Some embodiments disclosed herein relate to a method of treating a respiratory condition that is developed because of a coronavirus and/or a picornavirus infection that can include administering to a subject suffering from the respiratory condition and/or contacting a cell infected with the coronavirus and/or the picornavirus in a subject suffering from the respiratory condition with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a respiratory condition due to a coronavirus infection and/or a picornavirus infection with an effective amount of the compound, or a pharmaceutically acceptable salt thereof. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein for treating a respiratory condition due to a coronavirus infection and/or a picornavirus infection.

A subject infected with a coronavirus can be asymptotic. A coronavirus infection can manifest itself via one or more symptoms. Examples of symptoms include, but are not limited to, coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness and/or palpitations. A coronavirus infection can cause complications. A non-limiting list of complications include, but are not limited to, sinusitis, otitis media, pneumonia, acute respiratory distress syndrome, disseminated intravascular coagulation, pericarditis and/or kidney failure.

As with a coronavirus, a subject infected with a picornavirus can be asymptotic. Alternatively, a subject can exhibit one or more of symptoms. Examples of symptoms of a picornavirus infection include, but are not limited to, aseptic meningitis, rash, conjunctivitis, runny nose a headache a cough a fever a sore throat, chest and/or abdominal pain and paralysis. As provided herein, subjects infected with a norovirus can exhibit one or more the symptoms including, but not limited to, nausea, non-bloody diarrhea, vomiting and abdominal pain. An example of a complication that can be attributed to a norovirus infection is dehydration, including severe dehydration.

Various indicators for determining the effectiveness of a method for treating a coronavirus, picornavirus and/or norovirus infection are also known to those skilled in the art. Examples of suitable indicators include, but are not limited to, a reduction in viral load indicated by reduction in coronavirus (or load) (e.g., reduction <10⁵ copies/mL in serum), a reduction in plasma viral load, a reduction in viral replication, a reduction in time to seroconversion (virus undetectable in patient serum), an increase in the rate of sustained viral response to therapy a reduction of morbidity or mortality in clinical outcomes, reduction in the need for a ventilator and/or total time on a ventilator, reduction in hospitalization rates and/or reduction in time in an ICU (intensive care unit) and/or hospital.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, camels, non-human primates, such as monkeys, chimpanzees, and apes, and, in particular, humans. In some embodiments, the subject can be human, for example, a human subject that is 60 years old or older.

The term “effective amount” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

In some embodiments, the subject can be asymptomatic, for example, the subject can be infected with coronavirus but does not exhibit any symptoms of the viral infection. In some embodiments, the subject can be have a pre-existing condition, such as asthma, hypertension, immunocompromised subjects (such as subjects with cancer, HIV and/or genetic immune deficiencies, bone marrow transplant subjects, solid organ transplant subjects, subjects who have had stem cells for cancer treatment and/or subjects who use oral or intravenous corticosteroids or other medicines called immunosuppressants), liver disease, subjects at risk for severe illness, chronic kidney disease being treated with dialysis, chronic lung disease, diabetes, hemoglobin disorders, serious heart conditions (for example, heart failure, coronary artery disease, congenital heart disease, cardiomyopathies, and pulmonary hypertension), severe obesity (such as subjects with a body mass index (BMI) of 40 or above) and people who live in a nursing home or long-term care facility. Additional examples and/or further information is provided by the CDC (https://www.cdc.gov/coronavirus/2019-ncov/need-extra-precautions/groups-at-higher-risk.html).

A compound described herein, including a compound of Formula (I), or a pharmaceutically acceptable salt thereof, can be administered after a subject is infected with a coronavirus. In addition and/or alternatively, a compound described herein, including a compound of Formula (I), or a pharmaceutically acceptable salt thereof, can be administered prophylactically.

Examples of agents that have been used to treat a coronavirus infection include Remdesivir. However, there can be drawbacks associated with compounds being used to treat a coronavirus including, but not limited to, one or more adverse side effects, the need for subcutaneous administration and/or high cost. Potential advantages of a compound of Formula (I), or a pharmaceutically acceptable salt thereof, can be less adverse side effects, delay in the onset of an adverse side effect and/or reduction in the severity of an adverse side effect.

A coronavirus infection can be treated by inhibiting certain mechanisms. In some embodiments, a compound described herein (such as a compound of Formula (I), or a pharmaceutically acceptable salt thereof) can be selective for a coronavirus protease. For example, a compound of Formula (I), or a pharmaceutically acceptable salt thereof, can be selective for a coronavirus protease compared to a host protease, for example, one or more host proteases selected from Cathepsin L, Cathepsin B, Cathepsin D, Cathepsin K, Leukocyte Elastase, Chymotrypsin, Trypsin, Thrombin, Pepsin, Caspase 2, Elastase and Calpain. In some embodiments, the selectivity for a coronavirus protease over a host protease (such as those described herein) can be >2-fold. In some embodiments, the selectivity for a coronavirus protease over a host protease (such as those described herein) can be >10-fold. In some embodiments, the selectivity for a coronavirus protease over a host protease (such as those described herein) can be >100-fold.

Studies have shown that the entry of SARS-CoV-2 into the target cells is a process that can be mediated by multiple proteases including cysteine cathepsins L and/or transmembrane protease serine 2 (TMPRSS2) (Shang et al., PNAS (2020) 117:11727, and Hoffmann et al., Cell (2020) 181:271-280). The cathepsin L inhibitor K117777, which lacks an inhibitory effect on the 3Clpro, can result in potent inhibition of SARS-CoV-2 in VeroE6, A549-ACE2 and/or HeLa-ACE2 (Mellott et al., bioRxiv (2020) 2020.2010.2023.347534). It has also been shown that the potent antiviral effect of K117777 is abolished when TMPRSS2 was expressed In A549-ACE2 (Steuten et al., bioRxiv (2020) 2020.2011.2021.392753). Off target activity of 3cLpro inhibitors, for example, on cathepsin L, may lead to an inaccurate assessment of the 3cLpro component of a compound's cellular potency. As an example, a compound described herein (such as a compound of Formula (I), or a pharmaceutically acceptable salt thereof) can have greater selectivity for a coronavirus protease over a host protease, such as cathepsin L. The selectivity can be determined by those skilled in the art, for example, using IC₅₀ and/or Ki values. In some embodiments, a compound described herein does not significantly inhibit cathepsin L (for example, IC₅₀ ≥10000 nM or >3.3 μM), but inhibits a coronavirus protease (for example, SARS-Cov-2 3Clpro).

A drawback with anti-viral treatment can be the development of resistance, including cross-resistance. Resistance can be a cause for treatment failure. The term “resistance” as used herein refers to a viral strain displaying a delayed, lessened and/or null response to an anti-viral agent. In some embodiments, a compound, or a pharmaceutically acceptable salt thereof, as described herein can be provided to a subject infected with a coronavirus strain that is resistant to one or more other anti-viral agents. In some embodiments, development of coronavirus resistant strains is delayed when a subject is treated with a compound, or a pharmaceutically acceptable salt thereof, as described herein compared to the development of a coronavirus resistant strain when treated with one or more other anti-viral agents.

### Combination Therapies

In some embodiments, a compound, or a pharmaceutically acceptable salt thereof, as described herein can be used in combination with one or more additional agent(s) for treating and/or inhibiting replication a coronavirus. Additional agents include, but are not limited to, an ACE inhibitor, an anticoagulant, an anti-inflammatory, an ARB, an ASO, a Covid-19 convalescent plasma, an entry inhibitor, an H₂ pump antagonist, an H-conducting channel, an HIV protease inhibitor, an HMG-CoA reductase inhibitor, an immune globulin, an immunosuppressant, an immunotherapeutic agent, a monoclonal antibody, a neuraminidase inhibitor, a nucleoside inhibitor, a nucleoside analog inhibitor, a polymerase inhibitor, a protease inhibitor, an siRNA, a statin, a tissue plasminogen activator, an antibiotic, an antimicrobial and a vaccine. Examples of additional agents include Ascorbic acid, Anakin, Azithromycin, Baloxavir, Baricitinib, Chloroquine Phosphate, Colchicine, a corticosteroid, Epoprostenol, Famotidine, Favipiravir, an IGIV, an interferon (for example, recombinant interferon alpha 2b, IFN-α and/or PEG-IFN-α-2a), an IVIG, Ivermectin, γ-globulin, lopinavir, Methylprednisolone, Molnupiravir (MK-4482 or EIDD-2801), Niclosamide, Nitazoxanide, Nitric oxide, Oseltamivir, Peramivir, RANTES, ribavirin, Remdesivir, Ruxolitinib, Sarilumab, Siltuximab, Sirolimus, a statin, Tacrolimus, Tocilizumab, Umifenovir, Zanamivir, Casirivimab, imdevimab, bamlanivimab, etesevimab and AT-527 (Good et al., Antimicrobial Agents and Chemotherapy (2021) 65(4):e02479-20)

In some embodiments, a compound, or a pharmaceutically acceptable salt thereof, as described herein can be administered with one or more additional agent(s) together in a single pharmaceutical composition. In some embodiments, a compound, or a pharmaceutically acceptable salt thereof, can be administered with one or more additional agent(s) as two or more separate pharmaceutical compositions. Further, the order of administration of a compound, or a pharmaceutically acceptable salt thereof, as described herein with one or more additional agent(s) can vary.

### EXAMPLES

Additional embodiments are disclosed in further detail in the following examples, which are not in any way intended to limit the scope of the claims.

Compounds

Compounds of Formula (I), along with pharmaceutically acceptable salts thereof, can be prepared in various ways, including those synthetic schemes shown and described herein, are provided below. Those skilled in the art will be able to recognize modifications of the disclosed syntheses and to devise routes based on the disclosures herein; all such modifications and alternate routes are within the scope of the claims.

### Synthesis of Intermediates

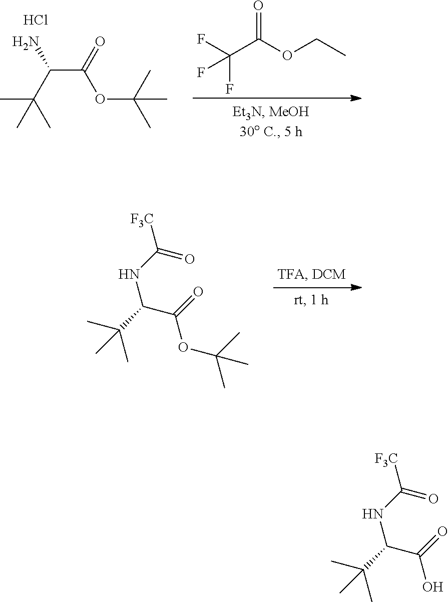

To a stirred mixture of t-butyl (2S)-2-amino-3,3-dimethylbutanoate hydrochloride (6.00 g, 26.8 mmol) and ethyl 2,2,2-trifluoroacetate (7.62 g, 53.6 mmol) in methanol (MeOH) (100 mL) was added triethylamine (5.43 g, 53.7 mmol) at 0° C. The mixture was stirred for 5 h at 30° C. and then concentrated under reduced pressure to afford the crude product that was diluted with dichloromethane (DCM) (150 mL) and made into a slurry with 100 ˜200 silica gel mesh (15 g), and the slurry was loaded to a column chromatography after removing the DCM. The sample was purified by column chromatography (Column size 6×24 cm, column volume: 600 mL, silica gel size (100 ˜200 mesh) quantity: 330 g) and eluted with MeOH:DCM (0% ˜10% over 30 min). The collected fractions: 0% MeOH:DCM fractions were chosen as the pure fractions. and those fractions were combined and concentrated under reduced pressure to provide t-butyl (2S)-3,3-dimethyl-2-(2,2,2-trifluoroacetamido)butanoate (7.20 g, 90%) as a white solid. ¹H NMR (300 MHz, CDCl₃) δ 6.78-6.90 (m, 1H), 4.32-4.38 (m, 1H), 1.50 (s, 9H), 1.01 (s, 9H). LC-MS (ESI, m/z): 282 [M−H]⁻.

To a mixture of t-butyl (2S)-3,3-dimethyl-2-(2,2,2-trifluoroacetamido)butanoate (1.03 g, 3.64 mmol) in DCM (5 mL) was added trifluoroacetic acid (5 mL). The mixture was stirred for 1 h at rt and then concentrated under reduced pressure to (2S)-3,3-dimethyl-2-(2,2,2-trifluoroacetamido)butanoic acid (826 mg, crude) as a yellow oil. LC-MS (ESI, m/z): 226 [M−H]⁻.

### Example 1

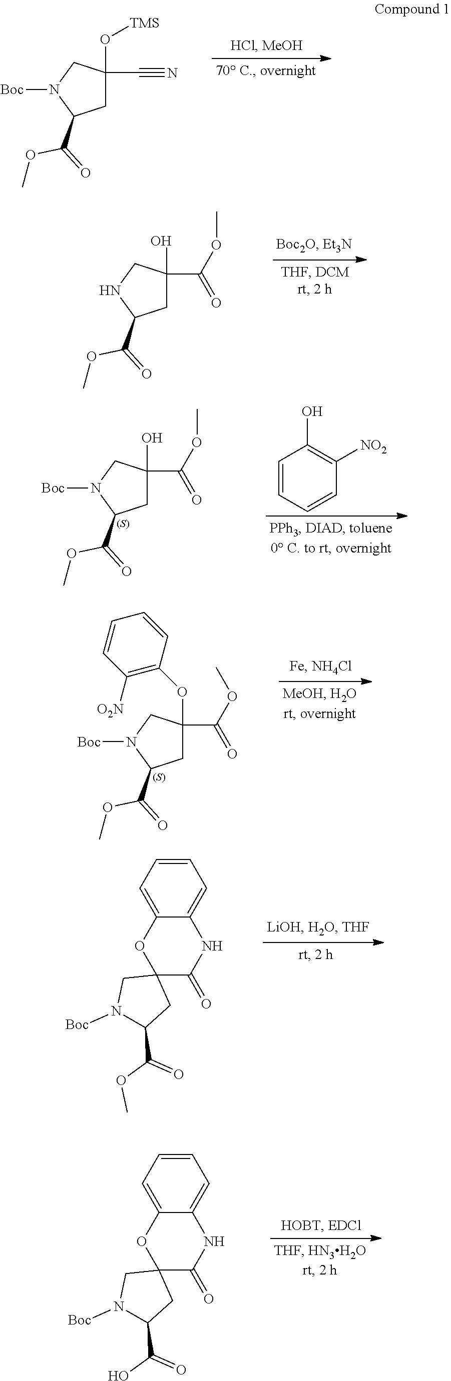

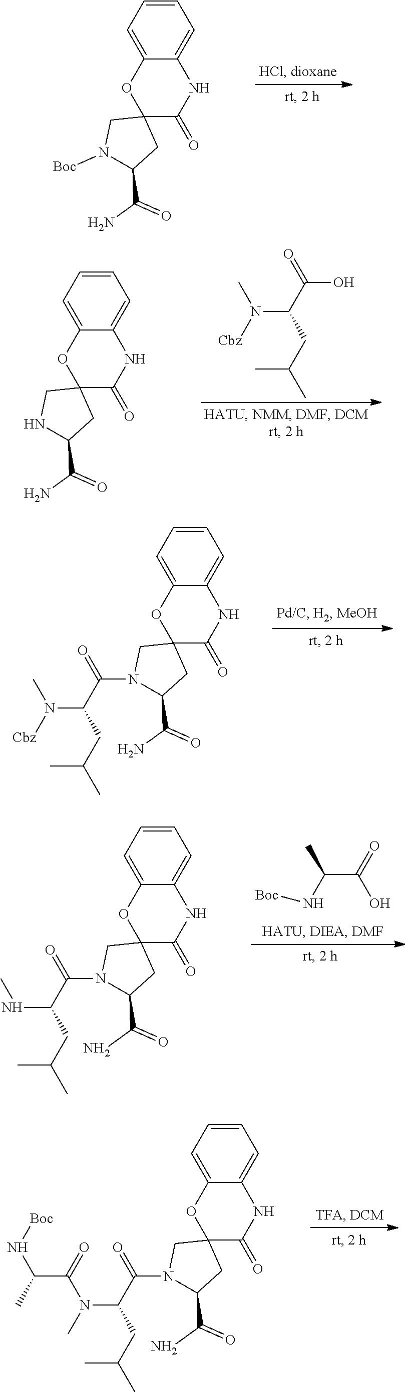

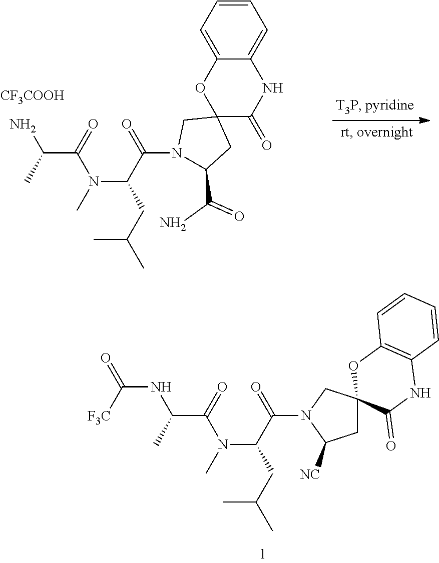 [SMILES: "CF3COOH" ; CC(C)C[C@@H](C(=O)N1CC2(C[C@H]1C(N)=O)Oc1ccccc1NC2=O)N(C)C(=O)[C@H](C)N ; CC(C)C[C@@H](C(=O)N1C[C@@]2(C[C@H]1C#N)Oc1ccccc1NC2=O)N(C)C(=O)[C@H](C)NC(=O)C(F)(F)F]

The mixture of 1-(t-butyl) 2-methyl (2S)-4-cyano-4-((trimethylsilyl)oxy)pyrrolidine-1,2-dicarboxylate (7.00 g, 23.1 mmol) in hydrochloride (90 mL, 4M in MeOH) was stirred for overnight at 70° C. The mixture was concentrated under reduced pressure to afford dimethyl (2S)-4-hydroxypyrrolidine-2,4-dicarboxylate (4.69 g, crude) as a red oil. LC-MS (ESI, m/z): 204 [M+H]⁺.

To a mixture of dimethyl (2S)-4-hydroxypyrrolidine-2,4-dicarboxylate (4.69 g, 23.1 mmol) in THF (40 mL) and DCM (106 mL), triethylamine (11.5 g, 113 mmol) was added di-t-butyl dicarbonate (12.4 g, 56.7 mmol). The mixture stirred for 2 h at room temperature (rt). The reaction was quenched with water (200 mL) and extracted with EA (3×300 mL). The organic layers were combined, washed with brine (2×100 mL) and dried over anhydrous Na₂SO₄. The solids were removed by filtration and the filtrate was concentrated under reduced pressure to afford the crude product that was chromatographed on a silica gel column with ethyl acetate (EA):petroleum ether (PE) (1:2) to provide 1-(t-butyl) 2,4-dimethyl (2S)-4-hydroxypyrrolidine-1,2,4-tricarboxylate (4.40 g, 63%) as a red oil. LC-MS (ESI, m/z): 304 [M+H]⁺.

To a stirred mixture of 1-(t-butyl) 2,4-dimethyl (2S)-4-hydroxypyrrolidine-1,2,4-tricarboxylate (500 mg, 1.65 mmol) in toluene (5 mL) was added o-nitrophenol (229 mg, 1.65 mmol) and triphenylphosphane (519 mg, 1.98 mmol) at 0° C. under nitrogen. The mixture was stirred for 20 min at 0° C., then diisopropyl azodicarboxylate (400 mg, 1.98 mmol) was added. The mixture was stirred overnight at rt. The reaction was quenched with water (30 mL). The mixture was extracted with EA (3×80 mL). The organic layers were combined, washed with brine (2×40 mL) and dried over anhydrous Na₂SO₄. The solids were removed by filtration and the filtrate was concentrated under reduced pressure to afford the crude product that was chromatographed on a silica gel column with EA:PE (1:1) to provide 1-(t-butyl) 2,4-dimethyl (2S)-4-(2-nitrophenoxy)pyrrolidine-1,2,4-tricarboxylate (460 mg, 66%) as a red oil. LC-MS (ESI, m/z): 425 [M+H]⁺.

To a mixture of 1-(t-butyl) 2,4-dimethyl (2S)-4-(2-nitrophenoxy)pyrrolidine-1,2,4-tricarboxylate (460 mg, 1.08 mmol) in MeOH (6 mL) and water (1.5 mL) was added NH₄Cl (137 mg, 2.59 mmol) and iron (307 mg, 5.47 mmol). The mixture was stirred for overnight at rt. The mixture was filtered through a celite pad and washed with DCM (3×50 mL). The organic layers were concentrated under reduced pressure to afford 1′-(t-butyl) 5′-methyl (5'S)-3-oxo-3,4-dihydrospiro[benzo[b][1,4]oxazine-2,3′-pyrrolidine]-1′,5′-dicarboxylate (350 mg, crude) as a red oil. LC-MS (ESI, m/z): 363 [M+H]⁺.

To a stirred mixture of 1′-(t-butyl) 5′-methyl (5′S)-3-oxo-3,4-dihydrospiro[benzo[b][1,4]oxazine-2,3′-pyrrolidine]-1′,5′-dicarboxylate (350 mg, 0.967 mmol) in THF (3 mL) and water (3 mL) was added LiOH (116 mg, 4.84 mmol) at rt. The mixture was stirred for 2 h and acidified to pH=3 with HCl (2M). The aqueous layer was extracted with EA (3×100 mL). The organic layers were combined, washed with brine (2×30 mL), dried over anhydrous Na₂SO₄ and concentrated under reduced pressure to afford (5′S)-1′-(t-butoxycarbonyl)-3-oxo-3,4-dihydrospiro[benzo[b][1,4]oxazine-2,3′-pyrrolidine]-5′-carboxylic acid (240 mg, crude) as a yellow oil. LC-MS (ESI, m/z): 349 [M+H]⁺.

To a mixture of (5'S)-1′-(t-butoxycarbonyl)-3-oxo-3,4-dihydrospiro[benzo[b][1,4]oxazine-2,3′-pyrrolidine]-5′-carboxylic acid (240 mg, 0.689 mmol) in THF (2.5 mL) was added 1-hydroxybenzotriazole (280 mg, 2.07 mmol) and 1-(3-dimethylaminopropyl)-3-ethylcarbodiimide hydrochloride (291 mg, 1.56 mmol). The mixture stirred for 30 min at 0° C. Ammonia (4.8 mL) was added at 0° C. and the mixture was stirred for 2 h at rt. The mixture was purified by C18 column with CH₃CN/water (0.05% NH₄HCO₃). The fraction was concentrated under reduced pressure to provide t-butyl (5'S)-5′-carbamoyl-3-oxo-3,4-dihydrospiro[benzo[b][1,4]oxazine-2,3′-pyrrolidine]-1′-carboxylate (130 mg, 54%) as a yellow solid. LC-MS (ESI, m/z): 348 [M+H]⁺.

A mixture of t-butyl (5'S)-5′-carbamoyl-3-oxo-3,4-dihydrospiro[benzo[b][1,4]oxazine-2,3′-pyrrolidine]-1′-carboxylate (130 mg, 0.375 mmol) in hydrochloride (40 mL, 4M in 1,4-dioxane) was stirred for 2 h at rt. The mixture was concentrated under reduced pressure to afford (5'S)-3-oxo-3,4-dihydrospiro[benzo[b][1,4]oxazine-2,3′-pyrrolidine]-5′-carboxamide (90 mg, crude) as a yellow solid. LC-MS (ESI, m/z): 248 [M+H]⁺.

To a stirred mixture of (5'S)-3-oxo-3,4-dihydrospiro[benzo[b][1,4]oxazine-2,3′-pyrrolidine]-5′-carboxamide (90.0 mg, 0.364 mmol), N-((benzyloxy)carbonyl)-N-methyl-L-leucine (104 mg, 0.375 mmol) and O-(7-azabenzotriazol-1-yl)-N,N,N′,N′-tetramethyluronium hexafluorophosphate (HATU) (143 mg, 0.375 mmol) in DCM (2 mL) and DMF (0.5 mL) was added N-methylmorpholine (110 mg, 1.09 mmol). The mixture was stirred for 2 h at rt. The reaction was quenched with water (10 mL). The mixture was extracted with EA (3×30 mL). The organic layers were combined, washed with brine (2×10 mL), dried over anhydrous Na₂SO₄, filtered and concentrated under reduced pressure to afford the crude product that was chromatographed on a silica gel column with MeOH:DCM (1:15) to provide benzyl ((2S)-1-((5'S)-5′-carbamoyl-3-oxo-3,4-dihydrospiro[benzo[b][1,4]oxazine-2,3′-pyrrolidin]-1′-yl)-4-methyl-1-oxopentan-2-yl)(methyl)carbamate (100 mg, 54%) as a white solid. LC-MS (ESI, m/z): 509 [M+H]⁺.

To a stirred mixture of benzyl ((2S)-1-((5'S)-5′-carbamoyl-3-oxo-3,4-dihydrospiro[benzo[b][1,4]oxazine-2,3′-pyrrolidin]-1′-yl)-4-methyl-1-oxopentan-2-yl)(methyl)carbamate (100 mg, 0.197 mmol) in methanol (4 mL) was added 10% Pd/C (28 mg). The mixture was stirred for 2 h at rt under hydrogen. The mixture was filtered through a celite pad and washed with DCM (3×30 mL). The filtrate was concentrated under reduced pressure to afford (5'S)-1′-(methyl-L-leucyl)-3-oxo-3,4-dihydrospiro[benzo[b][1,4]oxazine-2,3′-pyrrolidine]-5′-carboxamide (80.0 mg, crude) as a yellow solid. LC-MS (ESI, m/z): 375 [M+H]⁺.

To a stirred mixture of (5'S)-1′-(methyl-L-leucyl)-3-oxo-3,4-dihydrospiro[benzo[b][1,4]oxazine-2,3′-pyrrolidine]-5′-carboxamide (73.7 mg, 0.197 mmol), (t-butoxycarbonyl)-L-alanine (37.2 mg, 0.197 mmol) and HATU (89.8 mg, 0.236 mmol) in DMF (2 mL) was added DIEA (152 mg, 1.18 mmol) at 0° C. The mixture was stirred for 2 h at rt. The reaction was quenched with water (5 mL). The mixture was purified by C18 column with CH₃CN/water (0.05% TFA). The fraction was concentrated under reduced pressure to provide t-butyl ((2S)-1-(((2S)-1-((5'S)-5′-carbamoyl-3-oxo-3,4-dihydrospiro[benzo[b][1,4]oxazine-2,3′-pyrrolidin]-1′-yl)-4-methyl-1-oxopentan-2-yl)(methyl)amino)-1-oxopropan-2-yl)carbamate (90 mg, 83%) as a yellow solid. LC-MS (ESI, m/z): 546 [M+H]⁺.

To a stirred mixture of t-butyl ((2S)-1-(((2S)-1-((5'S)-5′-carbamoyl-3-oxo-3,4-dihydrospiro[benzo[b][1,4]oxazine-2,3′-pyrrolidin]-1′-yl)-4-methyl-1-oxopentan-2-yl)(methyl)amino)-1-oxopropan-2-yl)carbamate (90.0 mg, 0.165 mmol) in DCM (1 mL) was added trifluoroacetic acid (0.3 mL). The mixture was stirred for 2 h at rt and then concentrated under reduced pressure to afford (5'S)-1′-(N-(L-alanyl)-N-methyl-L-leucyl)-3-oxo-3,4-dihydrospiro[benzo[b][1,4]oxazine-2,3′-pyrrolidine]-5′-carboxamide 2,2,2-trifluoroacetate (92 mg, crude) as a yellow oil. LC-MS (ESI, m/z): 446 [M+H]⁺.

To a stirred mixture of (5'S)-1′-(N-(L-alanyl)-N-methyl-L-leucyl)-3-oxo-3,4-dihydrospiro[benzo[b][1,4]oxazine-2,3′-pyrrolidine]-5′-carboxamide 2,2,2-trifluoroacetate (92.0 mg, 0.165 mmol) and 2,4,6-tripropyl-1,3,5,2,4,6-trioxatriphosphorinane-2,4,6-trioxide (1.05 g, 1.65 mmol) was added pyridine (65.2 mg, 0.825 mmol) at rt. The mixture was stirred overnight at rt. The reaction was quenched with water (5 mL). The mixture was extracted with EA (3×40 mL). The organic layers were combined, washed with brine (2×10 mL), dried over anhydrous Na₂SO₄, filtered and concentrated under reduced pressure to afford the crude product that was purified by prep-HPLC (Column: Xselect CSH Prep C18 OBD Column, 19×250 mm, 5 m; Mobile Phase A: water (0.1% FA), Mobile Phase B: acetonitrile (ACN); Flow rate: 25 mL/min; Gradient: 35% B to 67% B in 7 min, 65% B; Wave Length: 220 nm; RT1 (min): 6) to provide (S)—N—((S)-1-((2R,5′S)-5′-cyano-3-oxo-3,4-dihydrospiro[benzo[b][1,4]oxazine-2,3′-pyrrolidin]-1′-yl)-4-methyl-1-oxopentan-2-yl)-N-methyl-2-(2,2,2-trifluoroacetamido)propanamide (6.8 mg, 8%, major isomer) as a white solid. ¹H NMR (400 MHz, 100° C., DMSO-d₆) δ 10.55-10.90 (m, 1H), 9.05-9.40 (m, 1H), 6.85-7.10 (m, 4H), 4.90-5.30 (m, 2H), 4.60-4.70 (m, 1H), 3.80-4.15 (m, 2H), 3.90-3.93 (m, 3H), 3.60-3.80 (m, 2H), 1.40-1.70 (m, 3H), 1.05-1.40 (m, 3H), 0.80-1.00 (m, 6H). LC-MS (ESI, m/z): 524 [M+H]⁺.

### Example 2

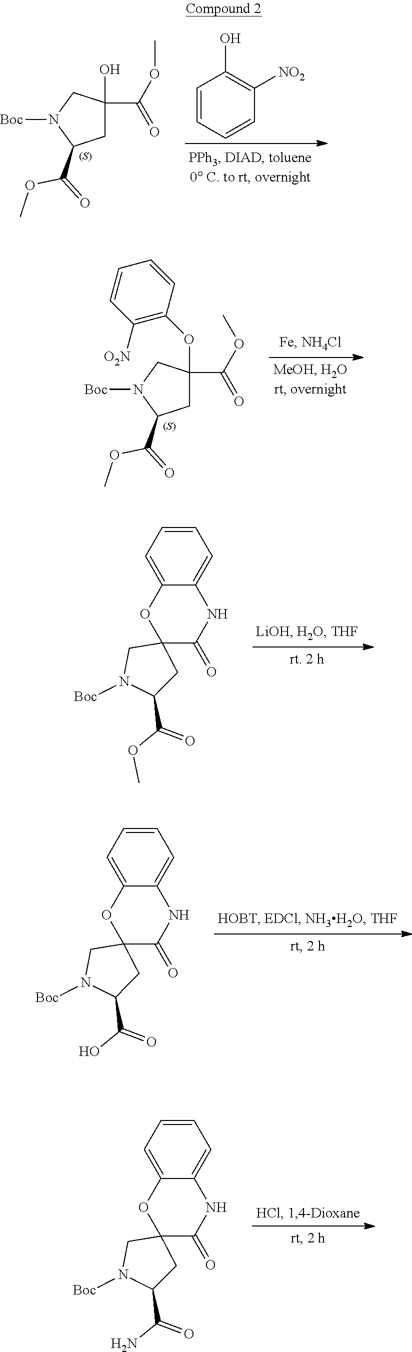

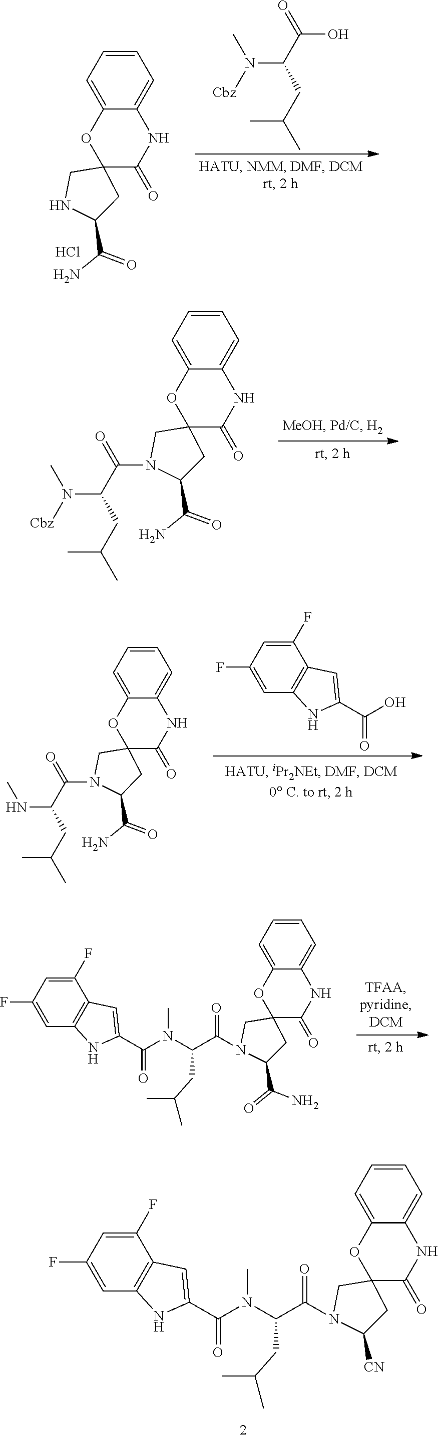

To a mixture of 1-(t-butyl) 2,4-dimethyl (2S)-4-hydroxypyrrolidine-1,2,4-tricarboxylate (800 mg, 2.64 mmol), o-nitrophenol (367 mg, 2.64 mmol) in toluene (8 mL) was added triphenylphosphane (997 mg, 3.17 mmol) at 0° C. under nitrogen. The mixture was stirred for 20 min at 0° C. Diisopropyl azodicarboxylate (768 mg, 3.17 mmol) was then added at 0° C. for 20 min. The mixture was stirred for overnight at rt. The reaction was quenched with water (30 mL). The mixture was extracted with EA (3×80 mL). The organic layers were combined, washed with brine (2×40 mL) and dried over anhydrous Na₂SO₄. The solids were removed by filtration and the filtrate was concentrated under reduced pressure to afford the crude product that was chromatographed on a silica gel column with EA:PE (1:1) to provide 1-(t-butyl) 2,4-dimethyl (2S)-4-(2-nitrophenoxy)pyrrolidine-1,2,4-tricarboxylate (2.00 g, crude) as a red oil. LC-MS (ESI, m/z): 425 [M+H]⁺.

To a mixture of 1-(t-butyl) 2,4-dimethyl (2S)-4-(2-nitrophenoxy)pyrrolidine-1,2,4-tricarboxylate (1.12 g, 2.64 mmol) and NH₄Cl (340 mg, 6.34 mmol) in MeOH (12 mL) and water (3 mL) was added iron (738 mg, 5.47 mmol) at rt. The mixture was stirred overnight, and then filtered through a celite pad and washed with DCM (3×50 mL). The organic layers were concentrated under reduced pressure to afford 1′-(t-butyl) 5′-methyl (5'S)-3-oxo-3,4-dihydrospiro[benzo[b][1,4]oxazine-2,3′-pyrrolidine]-1′,5′-dicarboxylate (2.81 g, crude) as a red oil. LC-MS (ESI, m/z): 363 [M+H]⁺.

To a stirred of 1′-(t-butyl) 5′-methyl (5'S)-3-oxo-3,4-dihydrospiro[benzo[b][1,4]oxazine-2,3′-pyrrolidine]-1′,5′-dicarboxylate (956 mg, 2.64 mmol) in THF (9 mL) and water (9 mL) was added LiOH (317 mg, 13.2 mmol) at rt. The mixture was stirred for 2 h and acidified to pH=3 with HCl (2M). The aqueous layer was extracted with EA (3×100 mL), dried over anhydrous Na₂SO₄ and concentrated under reduced pressure to afford (5'S)-1′-(t-butoxycarbonyl)-3-oxo-3,4-dihydrospiro[benzo[b][1,4]oxazine-2,3′-pyrrolidine]-5′-carboxylic acid (730 mg, 76%) as a yellow oil. LC-MS (ESI, m/z): 349 [M+H]⁺.

To a mixture of (5'S)-1′-(t-butoxycarbonyl)-3-oxo-3,4-dihydrospiro[benzo[b][1,4]oxazine-2,3′-pyrrolidine]-5′-carboxylic acid (730 mg, 2.09 mmol) in THF (7 mL) were added 1-hydroxybenzotriazole (847 mg, 6.27 mmol) and 1-(3-dimethylaminopropyl)-3-ethylcarbodiimide hydrochloride (878 mg, 4.60 mmol). The mixture was stirred for 30 min at 0° C. Ammonia (14.6 mL) was added at 0° C. and the mixture was stirred for 2 h at rt. The mixture was purified by C18 column with CH₃CN/water (0.05% NH₄HCO₃). The fraction was concentrated under reduced pressure to provide t-butyl (5'S)-5′-carbamoyl-3-oxo-3,4-dihydrospiro[benzo[b][1,4]oxazine-2,3′-pyrrolidine]-1′-carboxylate (730 mg, crude) as a yellow solid. LC-MS (ESI, m/z): 348 [M+H]⁺.

A mixture of t-butyl (5'S)-5′-carbamoyl-3-oxo-3,4-dihydrospiro[benzo[b][1,4]oxazine-2,3′-pyrrolidine]-1′-carboxylate (725 mg, 2.09 mmol) in hydrochloride (30 mL, 4M in 1,4-dioxane) was stirred for 2 h at rt. The mixture was concentrated under reduced pressure to afford (5'S)-3-oxo-3,4-dihydrospiro[benzo[b][1,4]oxazine-2,3′-pyrrolidine]-5′-carboxamide hydrochloride (516 mg, crude) as a yellow solid. LC-MS (ESI, m/z): 248 [M+H]⁺.

To a stirred mixture of (5'S)-3-oxo-3,4-dihydrospiro[benzo[b][1,4]oxazine-2,3′-pyrrolidine]-5′-carboxamide hydrochloride (150 mg, 0.607 mmol), N-((benzyloxy)carbonyl)-N-methyl-L-leucine (174 mg, 0.625 mmol) and HATU (238 mg, 0.625 mmol) in DCM (1.6 mL) and DMF (0.4 mL) was added N-methylmorpholine (190 mg, 1.88 mmol). The mixture was stirred for 2 h at rt. The reaction was quenched with water (10 mL). The mixture was purified by C18 column with CH₃CN/water (0.05% TFA). The fraction was concentrated under reduced pressure to provide benzyl ((2S)-1-((5'S)-5′-carbamoyl-3-oxo-3,4-dihydrospiro[benzo[b][1,4]oxazine-2,3′-pyrrolidin]-1′-yl)-4-methyl-1-oxopentan-2-yl)(methyl)carbamate (150 mg, 49%) as a white solid. LC-MS (ESI, m/z): 509 [M+H]⁺.

To a stirred mixture of benzyl ((2S)-1-((5'S)-5′-carbamoyl-3-oxo-3,4-dihydrospiro[benzo[b][1,4]oxazine-2,3′-pyrrolidin]-1′-yl)-4-methyl-1-oxopentan-2-yl)(methyl)carbamate (150 mg, 0.296 mmol) in methanol (4 mL) was added 10% Pd/C (40.0 mg). The mixture was stirred for 2 h at rt under hydrogen. The mixture was filtered through a celite pad and washed with DCM (3×30 mL). The filtrate was concentrated under reduced pressure to afford (5'S)-1′-(methyl-L-leucyl)-3-oxo-3,4-dihydrospiro[benzo[b][1,4]oxazine-2,3′-pyrrolidine]-5′-carboxamide (100 mg, crude) as a yellow solid. LC-MS (ESI, m/z): 375 [M+H]⁺.

To a stirred mixture of (5'S)-1′-(methyl-L-leucyl)-3-oxo-3,4-dihydrospiro[benzo[b][1,4]oxazine-2,3′-pyrrolidine]-5′-carboxamide (100 mg, 0.267 mmol), 4,6-difluoro-1H-indole-2-carboxylic acid (60.5 mg, 0.307 mmol) and HATU (112 mg, 0.293 mmol) in DMF (1 mL) and DCM (1 mL) was added DIEA (103 mg, 0.801 mmol) at 0° C. The mixture was stirred for 2 h at rt. The reaction was quenched with water (5 mL). The mixture was purified by C18 column with CH₃CN/water (0.05% TFA). The fraction was concentrated under reduced pressure to provide (5'S)-1′-(N-(4,6-difluoro-1H-indole-2-carbonyl)-N-methyl-L-leucyl)-3-oxo-3,4-dihydrospiro[benzo[b][1,4]oxazine-2,3′-pyrrolidine]-5′-carboxamide (50.0 mg, crude) as a yellow solid. LC-MS (ESI, m/z): 554 [M+H]⁺.

To a mixture of (5'S)-1′-(N-(4,6-difluoro-1H-indole-2-carbonyl)-N-methyl-L-leucyl)-3-oxo-3,4-dihydrospiro[benzo[b][1,4]oxazine-2,3′-pyrrolidine]-5′-carboxamide (50.0 mg, 0.090 mmol) in DCM (1 mL) were added pyridine (35.6 mg, 0.450 mmol) and trifluoroacetic anhydride (37.8 mg, 0.180 mmol). The mixture was stirred for overnight at rt. The reaction was quenched with water (20 mL). The mixture was extracted with DCM (3×20 mL). The organic layers were combined, washed with brine (2×20 mL) and dried over anhydrous Na₂SO₄. The solids were removed by filtration and the filtrate was concentrated under reduced pressure to afford the crude product that was purified by prep-HPLC (Column: Xselect CSH F-Phenyl OBD column, 19×250 mm, 5 m; Mobile Phase A: Water (0.1% FA), Mobile Phase B: ACN; Flow rate: 25 mL/min; Gradient: 40% B to 67% B in 7 min, 67% B to 67% B in 8 min, 67% B; Wave Length: 220 nm; RT1 (min): 7.6) to provide N—((S)-1-((2R,5'S)-5′-cyano-3-oxo-3,4-dihydrospiro[benzo[b][1,4]oxazine-2,3′-pyrrolidin]-1′-yl)-4-methyl-1-oxopentan-2-yl)-4,6-difluoro-N-methyl-1H-indole-2-carboxamide (7.1 mg, 15%, major isomer) as a white solid. ¹H NMR (400 MHz, 100° C., DMSO-d₆) δ 11.40-12.00 (m, 1H), 10.50-11.00 (m, 1H), 6.90-7.15 (m, 2H), 6.85-6.90 (m, 2H), 6.70-6.85 (m, 2H), 6.50-6.70 (m, 1H), 5.20-5.70 (m, 1H), 4.80-5.20 (m, 1H), 3.80-4.40 (m, 2H), 3.10-3.50 (m, 3H), 2.60-2.90 (m, 2H), 1.50-2.00 (m, 3H), 0.70-1.20 (m, 6H). LC-MS (ESI, m/z): 536 [M+H]⁺.

### Example 3

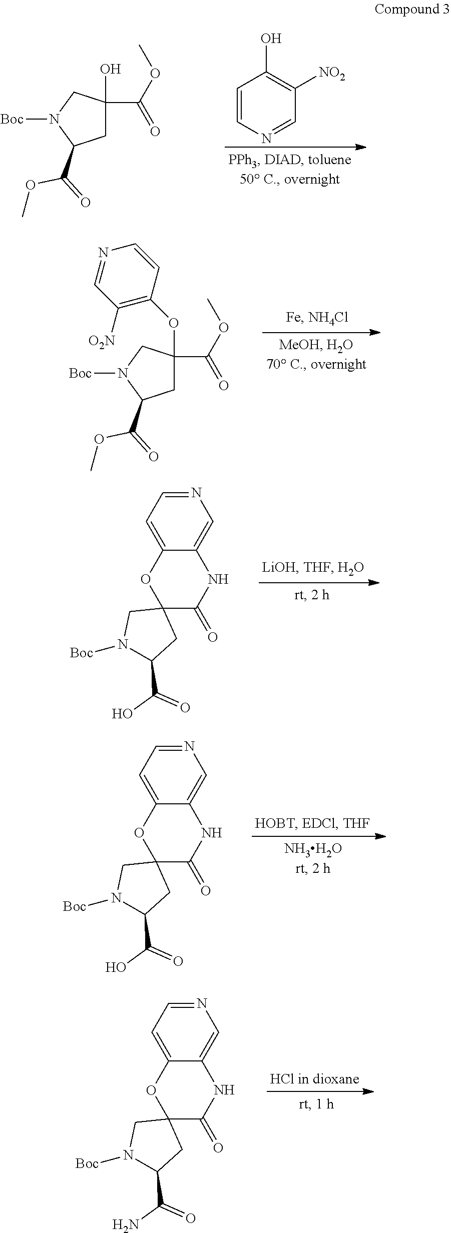

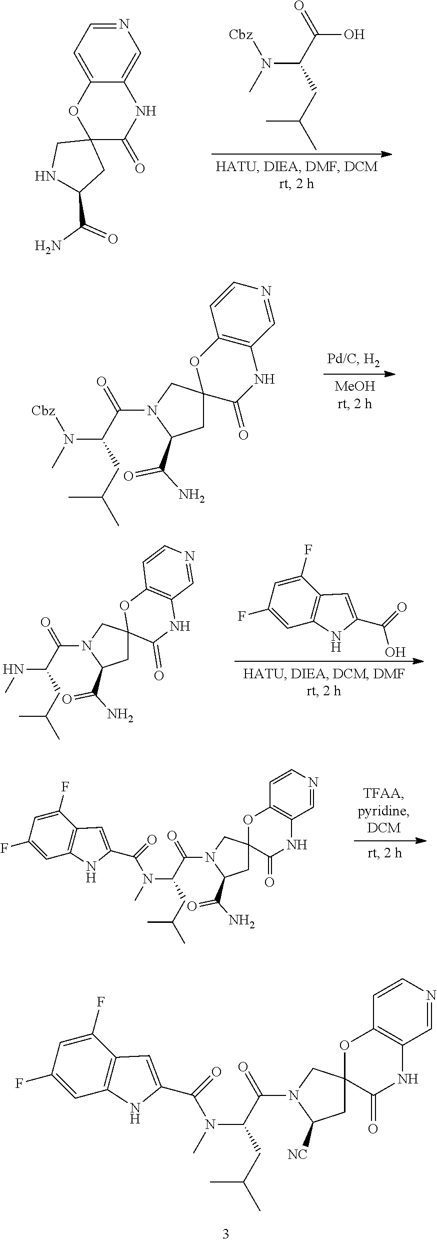

To a stirred mixture of 1-(t-butyl) 2,4-dimethyl (2S)-4-hydroxypyrrolidine-1,2,4-tricarboxylate (800 mg, 2.64 mmol) in toluene (8 mL) were added 3-nitropyridin-4-ol (370 mg, 2.64 mmol) and triphenylphosphane (830 mg, 3.17 mmol) at 0° C. under nitrogen. The mixture was stirred for 20 min at 0° C., and then diisopropyl azodicarboxylate (640 mg, 3.17 mmol) was added. The mixture was stirred for overnight at 50° C. under nitrogen. The reaction was quenched with water (40 mL). The mixture was extracted with EA (3×80 mL). The organic layers were combined, washed with brine (2×40 mL) and dried over anhydrous Na₂SO₄. The solids were removed by filtration and the filtrate was concentrated under reduced pressure to afford the crude product that was chromatographed on a silica gel column with EA:PE (1:1) to provide 1-(t-butyl) 2,4-dimethyl (2S)-4-((3-nitropyridin-4-yl)oxy)pyrrolidine-1,2,4-tricarboxylate (600 mg, crude) as a red oil. LC-MS (ESI, m/z): 426 [M+H]⁺.

The mixture of 1-(t-butyl) 2,4-dimethyl (2S)-4-((3-nitropyridin-4-yl)oxy)pyrrolidine-1,2,4-tricarboxylate (600 mg, 1.41 mmol) in MeOH (6 mL) and water (1.5 mL) were added NH₄Cl (181 mg, 3.38 mmol) and iron (394 mg, 7.05 mmol). The mixture was stirred for overnight at 70° C. The mixture was filtered through a celite pad and washed with DCM (3×60 mL). The organic layers were concentrated under reduced pressure to afford (5'S)-1′-(t-butoxycarbonyl)-3-oxo-3,4-dihydrospiro[pyrido[4,3-b][1,4]oxazine-2,3′-pyrrolidine]-5′-carboxylic acid (600 mg, crude) as a red oil. LC-MS (ESI, m/z): 364 [M+H]⁺.

To a stirred of (5'S)-1′-(t-butoxycarbonyl)-3-oxo-3,4-dihydrospiro[pyrido[4,3-b][1,4]oxazine-2,3′-pyrrolidine]-5′-carboxylic acid (600 mg, 1.65 mmol) in THF (6 mL) and water (6 mL) was added LiOH (198 mg, 8.26 mmol) at rt. The mixture was stirred for 2 h at rt and acidified to pH=3 with HCl (2M). The aqueous layer was extracted with EA (3×100 mL). The organic layers were combined, washed with brine (2×30 mL) and dried over anhydrous Na₂SO₄. The mixture was concentrated under reduced pressure to afford (5'S)-1′-(t-butoxycarbonyl)-3-oxo-3,4-dihydrospiro[pyrido[4,3-b][1,4]oxazine-2,3′-pyrrolidine]-5′-carboxylic acid (270 mg, crude) as a yellow oil. LC-MS (ESI, m/z): 350 [M+H]⁺.

To a mixture of (5'S)-1′-(t-butoxycarbonyl)-3-oxo-3,4-dihydrospiro[pyrido[4,3-b][1,4]oxazine-2,3′-pyrrolidine]-5′-carboxylic acid (270 mg, 0.773 mmol) in THF (3 mL) were added 1-hydroxybenzotriazole (314 mg, 2.40 mmol) and 1-(3-dimethylaminopropyl)-3-ethylcarbodiimide hydrochloride (326 mg, 1.70 mmol). The mixture stirred for 30 min at 0° C. Ammonia (5.4 mL) was added at 0° C. and the mixture stirred for 2 h at rt. The mixture was purified by C18 column with CH₃CN/water (0.05% NH₄HCO₃). The fraction was concentrated under reduced pressure to provide (5'S)-1′-(t-butoxycarbonyl)-3-oxo-3,4-dihydrospiro[pyrido[4,3-b][1,4]oxazine-2,3′-pyrrolidine]-5′-carboxylic acid (150 mg, crude) as a yellow solid. LC-MS (ESI, m/z): 349 [M+H]⁺.

A mixture of (5'S)-1′-(t-butoxycarbonyl)-3-oxo-3,4-dihydrospiro[pyrido[4,3-b][1,4]oxazine-2,3′-pyrrolidine]-5′-carboxylic acid (150 mg, 0.431 mmol) in hydrochloride (5 mL, 4 M in 1,4-dioxane) was stirred for 2 h at rt. The mixture was concentrated under reduced pressure to afford (5'S)-3-oxo-3,4-dihydrospiro[pyrido[4,3-b][1,4]oxazine-2,3′-pyrrolidine]-5′-carboxamide (100 mg, crude) as a yellow solid. LC-MS (ESI, m/z): 249 [M+H]⁺.

To a stirred mixture of (5'S)-3-oxo-3,4-dihydrospiro[pyrido[4,3-b][1,4]oxazine-2,3′-pyrrolidine]-5′-carboxamide (100 mg, 0.403 mmol), N-((benzyloxy)carbonyl)-N-methyl-L-leucine (116 mg, 0.415 mmol) and HATU (158 mg, 0.415 mmol) in DCM (1 mL) and DMF (0.25 mL) was added N-methylmorpholine (122 mg, 1.21 mmol). The mixture was stirred for 2 h at rt. The reaction was quenched with water (5 mL). The mixture was extracted with EA (3×30 mL). The organic layers were combined, washed with brine (2×10 mL), dried over anhydrous Na₂SO₄. The solids were removed by filtration and the filtrate was concentrated under reduced pressure to afford the crude product that was chromatographed on a silica gel column with MeOH:DCM (1:15) to provide benzyl ((2S)-1-((5'S)-5′-carbamoyl-3-oxo-3,4-dihydrospiro[pyrido[4,3-b][1,4]oxazine-2,3′-pyrrolidin]-1′-yl)-4-methyl-1-oxopentan-2-yl)(methyl)carbamate (90.0 mg, crude) as a white solid. LC-MS (ESI, m/z): 510 [M+H]⁺.

To a stirred mixture of benzyl ((2S)-1-((5'S)-5′-carbamoyl-3-oxo-3,4-dihydrospiro[pyrido[4,3-b][1,4]oxazine-2,3′-pyrrolidin]-1′-yl)-4-methyl-1-oxopentan-2-yl)(methyl)carbamate (90.0 mg, 0.177 mmol) in methanol (4 mL) was added 10% Pd/C (23 mg). The mixture was stirred for 2 h at rt under hydrogen. The mixture was filtered through a celite pad and washed with DCM (3×30 mL). The filtrate was concentrated under reduced pressure to afford (5'S)-1′-(methyl-L-leucyl)-3-oxo-3,4-dihydrospiro[pyrido[4,3-b][1,4]oxazine-2,3′-pyrrolidine]-5′-carboxamide (65.0 mg, crude) as a yellow solid. LC-MS (ESI, m/z): 376 [M+H]⁺.

To a stirred mixture of (5'S)-1′-(methyl-L-leucyl)-3-oxo-3,4-dihydrospiro[pyrido[4,3-b][1,4]oxazine-2,3′-pyrrolidine]-5′-carboxamide (65.0 mg, 0.173 mmol), 4,6-difluoro-1H-indole-2-carboxylic acid (39.3 mg, 0.199 mmol) and HATU (72.4 mg, 0.190 mmol) in DMF (1 mL) and DCM (1 mL) was added DIEA (67.1 mg, 0.519 mmol) at 0° C. The mixture was stirred for 2 h at rt. The reaction was quenched with water (5 mL). The mixture was purified by C18 column with CH₃CN/water (0.05% TFA). The fraction was concentrated under reduced pressure to provide (5'S)-1′-(N-(4,6-difluoro-1H-indole-2-carbonyl)-N-methyl-L-leucyl)-3-oxo-3,4-dihydrospiro[pyrido[4,3-b][1,4]oxazine-2,3′-pyrrolidine]-5′-carboxamide (40.0 mg, 42%) as a yellow solid. LC-MS (ESI, m/z): 555 [M+H]⁺.

To a mixture of (5'S)-1′-(N-(4,6-difluoro-1H-indole-2-carbonyl)-N-methyl-L-leucyl)-3-oxo-3,4-dihydrospiro[pyrido[4,3-b][1,4]oxazine-2,3′-pyrrolidine]-5′-carboxamide (40.0 mg, 0.072 mmol) in DCM (1 mL) were added pyridine (29.0 mg, 0.360 mmol) and trifluoroacetic anhydride (30.8 mg, 0.144 mmol). The mixture was stirred for overnight at rt. The reaction was quenched with water (20 mL). The mixture was extracted with DCM (3×20 mL). The organic layers were combined, washed with brine (2×20 mL) and dried over anhydrous Na₂SO₄. The solids were removed by filtration and the filtrate was concentrated under reduced pressure to afford the crude product that was purified by prep-HPLC (Column: Xselect CSH F-Phenyl OBD column, 19×250 mm, 5 m; Mobile Phase A: water (0.1% FA), Mobile Phase B: ACN; Flow rate: 25 mL/min; Gradient: 35% B to 67% B in 7 min, 65% B; Wave Length: 220 nm; RT1 (min): 6.88) to provide N—((S)-1-((2R,5'S)-5′-cyano-3-oxo-3,4-dihydrospiro[pyrido[4,3-b][1,4]oxazine-2,3′-pyrrolidin]-1′-yl)-4-methyl-1-oxopentan-2-yl)-4,6-difluoro-N-methyl-1H-indole-2-carboxamide (8.00 mg, 20%, major isomer) as an off-white solid. ¹H NMR (400 MHz, 100° C., DMSO-d₆) δ 11.45-11.80 (m, 1H), 10.90-11.20 (m, 1H), 8.10-8.20 (m, 1H), 7.70-7.85 (m, 1H), 6.70-7.15 (m, 4H), 4.90-5.50 (m, 2H), 3.85-4.30 (m, 2H), 3.20-3.30 (m, 3H), 2.60-2.90 (m, 2H), 1.50-1.90 (m, 3H), 0.80-1.05 (m, 6H). LC-MS (ESI, m/z): 537 [M+H]⁺.

### Example 4

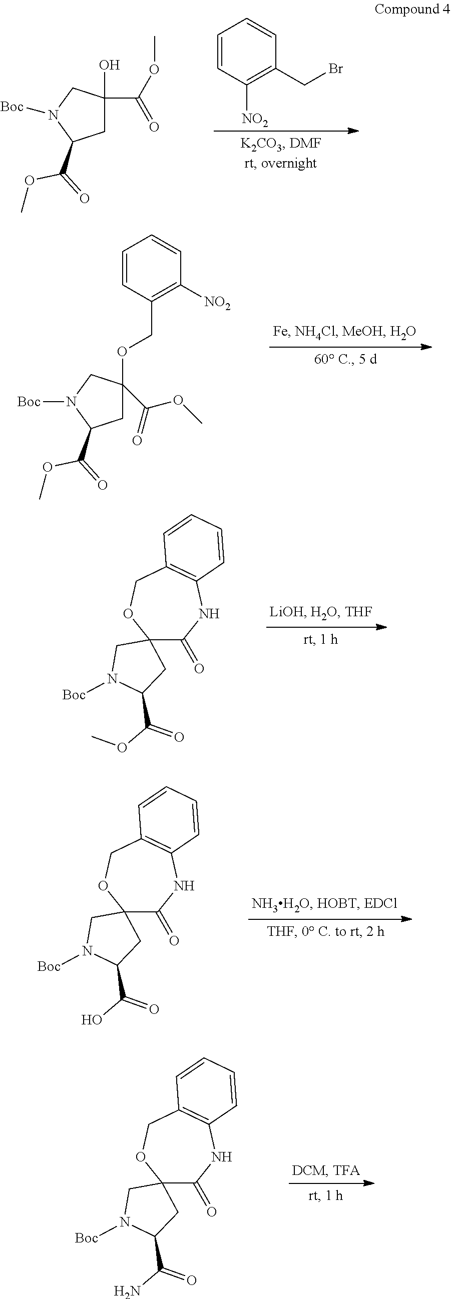 [SMILES: COC(=O)[C@@H]1CC(O)(C(=O)OC)CN1C(=O)OC(C)(C)C ; O=[N+]([O-])c1ccccc1CBr ; COC(=O)[C@@H]1CC(OCc2ccccc2[N+](=O)[O-])(C(=O)OC)CN1C(=O)OC(C)(C)C ; COC(=O)[C@@H]1CC2(CN1C(=O)OC(C)(C)C)OCc1ccccc1NC2=O ; CC(C)(C)OC(=O)N1CC2(C[C@H]1C(=O)O)OCc1ccccc1NC2=O ; CC(C)(C)OC(=O)N1CC2(C[C@H]1C(N)=O)OCc1ccccc1NC2=O]

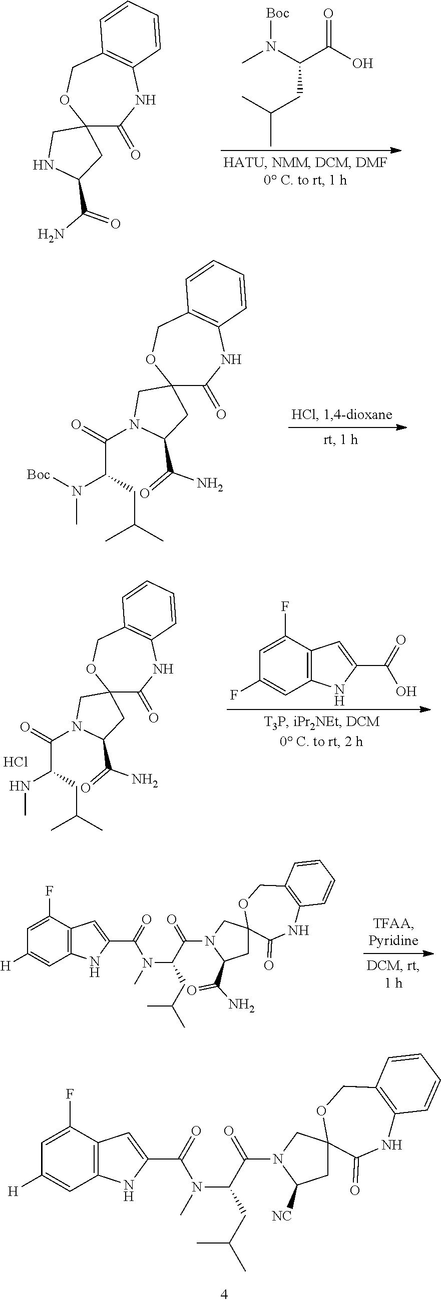

To a solution of 1-(t-butyl) 2,4-dimethyl (2S)-4-hydroxypyrrolidine-1,2,4-tricarboxylate (2.0 g, 6.59 mmol) in DMF (20 mL) were added 1-(bromomethyl)-2-nitrobenzene (4.27 g, 19.8 mmol) and K₂CO₃ (4.56 g, 33.0 mmol). The mixture was stirred overnight at rt. The mixture was filtered through a celite pad and washed with EA (3×50 mL). The filtrate was diluted with EA (150 mL), washed with brine (2×100 mL), dried over anhydrous Na₂SO₄. The solids were removed by filtration and the filtrate was concentrated under reduced pressure to afford the crude product that was chromatographed on a silica gel column with EA:PE (35%-40%) and then further purified by C18 column with CH₃CN/water (0.05% TFA). The fraction was concentrated under reduced pressure to provide 1-(t-butyl) 2,4-dimethyl (2S)-4-((2-nitrobenzyl)oxy)pyrrolidine-1,2,4-tricarboxylate (700 mg, 22%) as a yellow solid. LC-MS (ESI, m/z): 339 [M−Boc+H]⁺.

To a solution of 1-(t-butyl) 2,4-dimethyl (2S)-4-((2-nitrobenzyl)oxy)pyrrolidine-1,2,4-tricarboxylate (700 mg, 1.50 mmol) in methanol (16 mL) and water (4 mL) were added iron (446 mg, 7.99 mmol) and NH₄Cl (123 mg, 2.30 mmol) at rt. The mixture was stirred 5 d at 60° C. The mixture was filtered through a celite pad and washed with MeOH (3×50 mL). The filtrate was concentrated under reduced pressure to afford the crude product that was purified by C18 column with CH₃CN/water (0.05% TFA). The fraction was concentrated under reduced pressure to provide 1′-(t-butyl) 5′-methyl (5'S)-2-oxo-1,5-dihydro-2H-spiro[benzo[e][1,4]oxazepine-3,3′-pyrrolidine]-1′,5′-dicarboxylate (180 mg, 30%) as a yellow solid. LC-MS (ESI, m/z): 277 [M−Boc+H]⁺.

To a solution of 1′-(t-butyl) 5′-methyl (5'S)-2-oxo-1,5-dihydro-2H-spiro[benzo[e][1,4]193xazepane-3,3′-pyrrolidine]-1′,5′-dicarboxylate (180 mg, 0.478 mmol) in THF (3 mL) and water (2 mL) was added LiOH (40.7 mg, 1.20 mmol). The mixture was stirred for 1 h at rt and acidified to pH=5 with HCl (2M). The mixture was concentrated under reduced pressure to remove the THF and acidified to pH=5 with HCl (2 M). The mixture was extracted with EA (3×100 mL). The organic layers were combined and dried over anhydrous Na₂SO₄. The solids were removed by filtration and the filtrate was concentrated under reduced pressure to provide (5'S)-1′-(t-butoxycarbonyl)-2-oxo-1,5-dihydro-2H-spiro[benzo[e][1,4]193 xazepane-3,3′-pyrrolidine]-5′-carboxylic acid (120 mg, 69%) as a yellow solid. LC-MS (ESI, m/z): 263 [M−Boc+H]⁺.

To a solution of (5'S)-1′-(t-butoxycarbonyl)-2-oxo-1,5-dihydro-2H-spiro[benzo[e][1,4]oxazepine-3,3′-pyrrolidine]-5′-carboxylic acid (120 mg, 0.331 mmol) in THF (8 mL) were added 1-hydroxybenzotriazole (134 mg, 0.993 mmol) and 1-(3-dimethylaminopropyl)-3-ethylcarbodiimide hydrochloride (140 mg, 0.728 mmol) at 0° C. After stirred 1 h, NH₄OH (2 mL) was added. The mixture was stirred for 1 h at rt and then diluted with water (30 mL). The mixture was extracted with EA (3×80 mL). The organic layers were combined, washed with brine (2×40 mL) and dried over anhydrous Na₂SO₄. The solids were removed by filtration and the filtrate was concentrated under reduced pressure to afford t-butyl (5'S)-5′-carbamoyl-2-oxo-1,5-dihydro-2H-spiro[benzo[e][1,4]oxazepine-3,3′-pyrrolidine]-1′-carboxylate (100 mg, crude) as a light yellow solid. LC-MS (ESI, m/z): 262 [M−Boc+H]⁺.

To a stirred mixture of t-butyl (5'S)-5′-carbamoyl-2-oxo-1,5-dihydro-2H-spiro[benzo[e][1,4]oxazepine-3,3′-pyrrolidine]-1′-carboxylate (100 mg, 0.277 mmol) in DCM (1.5 mL) was added trifluoroacetic acid (0.5 mL) at rt. The mixture was stirred for 1 h at rt and concentrated under reduced pressure to afford (5'S)-2-oxo-1,5-dihydro-2H-spiro[benzo[e][1,4]oxazepine-3,3′-pyrrolidine]-5′-carboxamide (80 mg, crude) as a yellow solid. LC-MS (ESI, m/z): 262 [M+H]⁺.

To a solution of (5'S)-2-oxo-1,5-dihydro-2H-spiro[benzo[e][1,4]oxazepine-3,3′-pyrrolidine]-5′-carboxamide (80 mg, 0.306 mmol), (2R)-2-[(t-butoxycarbonyl) (methyl)amino]-4-methylpentanoic acid (90.1 mg, 0.367 mmol) and HATU (140 mg, 0.367 mmol) in DCM (1.8 mL) and DMF (0.3 mL) was added N-methylmorpholine (155 mg, 1.53 mmol) stirred at 0° C. The mixture was stirred for 1 h at rt. The reaction was quenched with water (30 mL). The mixture was extracted with EA (3×50 mL). The organic layers were combined, washed with brine (2×20 mL) and dried over anhydrous Na₂SO₄. The solids were removed by filtration and the filtrate was concentrated under reduced pressure to afford the crude product that was chromatographed on a silica gel column with EA:PE to provide t-butyl ((2S)-1-((5'S)-5′-carbamoyl-2-oxo-1,5-dihydro-2H-spiro[benzo[e][1,4]oxazepine-3,3′-pyrrolidin]-1′-yl)-4-methyl-1-oxopentan-2-yl)(methyl)carbamate (90 mg, 60%) as a colorless solid. LC-MS (ESI, m/z): 489 [M+H]⁺.

To a solution of t-butyl ((2S)-1-((5'S)-5′-carbamoyl-2-oxo-1,5-dihydro-2H-spiro[benzo[e][1,4]oxazepine-3,3′-pyrrolidin]-1′-yl)-4-methyl-1-oxopentan-2-yl)(methyl)carbamate (90 mg, 0.184 mmol) in 1,4-dioxane (1 mL) was added hydrogen chloride (2 mL, 4M in 1,4-dioxane) at rt. The mixture was stirred for 1 h at rt. The mixture was concentrated under reduced pressure to afford (5'S)-1′-(methyl-L-leucyl)-2-oxo-1,5-dihydro-2H-spiro[benzo[e][1,4]oxazepine-3,3′-pyrrolidine]-5′-carboxamide hydrochloride (70 mg, crude) as a light yellow solid. LC-MS (ESI, m/z): 389 [M+H]⁺.

To a solution of (5'S)-1′-(methyl-L-leucyl)-2-oxo-1,5-dihydro-2H-spiro[benzo[e][1,4]oxazepine-3,3′-pyrrolidine]-5′-carboxamide hydrochloride (80 mg, 0.188 mmol) and 4,6-difluoro-1H-indole-2-carboxylic acid (44.5 mg, 0.226 mmol) in DCM (2 mL) were added 2,4,6-tripropyl-1,3,5,2,4,6-trioxatriphosphorinane-2,4,6-trioxide (359 mg, 0.564 mmol, 50% wt in EA) and N-ethyl-N-isopropylpropan-2-amine (292 mg, 2.26 mmol) at 0° C. The mixture was stirred for 2 h at rt. The reaction waws quenched with water (20 mL). The mixture was extracted with DCM (3×50 mL). The organic layers were combined, washed with brine (2×20 mL) and dried over anhydrous Na₂SO₄. The solids were removed by filtration and the filtrate was concentrated under reduced pressure to afford the crude product that was purified by TLC (Mobile phase: EA; Rf=0.4; detection: UV) to provide (5'S)-1′-(N-(4,6-difluoro-1H-indole-2-carbonyl)-N-methyl-L-leucyl)-2-oxo-1,5-dihydro-2H-spiro[benzo[e][1,4]oxazepine-3,3′-pyrrolidine]-5′-carboxamide (40 mg, crude) as a dark yellow solid. LC-MS (ESI, m/z): 568 [M+H]⁺.

To a solution of (5'S)-1′-(N-(4,6-difluoro-1H-indole-2-carbonyl)-N-methyl-L-leucyl)-2-oxo-1,5-dihydro-2H-spiro[benzo [e][1,4]oxazepine-3,3′-pyrrolidine]-5′-carboxamide (40.0 mg, 0.073 mmol) in DCM (1 mL) were added pyridine (28.8 mg, 0.365 mmol) and trifluoroacetic anhydride (12.7 mg, 0.131 mmol). The mixture was stirred 1 h at rt. The reaction was quenched with water (10 mL). The mixture was extracted with DCM (3×20 mL). The organic layers were combined, washed with brine (2×20 mL) and dried over anhydrous Na₂SO₄. The solids were removed by filtration and the filtrate was concentrated under reduced pressure to afford the crude product that was purified by prep-HPLC (Column: Xselect CSH Prep C18 OBD Column, 19×150 mm, 5 m; Mobile Phase A: water (0.1% FA), Mobile Phase B: ACN; Flow rate: 25 mL/min; Gradient: 43% B to 73% B in 7 min, 73% B; Wave Length: 254 nm; RT1 (min): 6.8;) to provide N—((S)-1-((3R,5'S)-5′-cyano-2-oxo-1,5-dihydro-2H-spiro[benzo[e][1,4]oxazepine-3,3′-pyrrolidin]-1′-yl)-4-methyl-1-oxopentan-2-yl)-4,6-difluoro-N-methyl-1H-indole-2-carboxamide (8.4 mg, 22%) as a white solid. ¹H NMR (400 MHz, 100° C., DMSO-d₆) δ 11.62-11.90 (m, 1H), 10.20-10.33 (m, 1H), 7.20-7.30 (m, 2H), 7.08-7.12 (m, 1H), 7.00-7.07 (m, 2H), 6.90-6.99 (m, 1H), 6.70-6.78 (m, 1H), 5.21-5.49 (m, 1H), 4.49-5.03 (m, 1H), 4.76-4.89 (m, 1H), 4.54-4.60 (m, 1H), 3.71-4.11 (m, 2H), 3.19-3.23 (m, 3H), 2.51-2.90 (m, 2H), 1.66-1.78 (m, 2H), 1.54-1.60 (m, 1H), 0.86-1.01 (m, 6H). LC-MS (ESI, m/z): 550 [M+H]⁺.

### Example 5

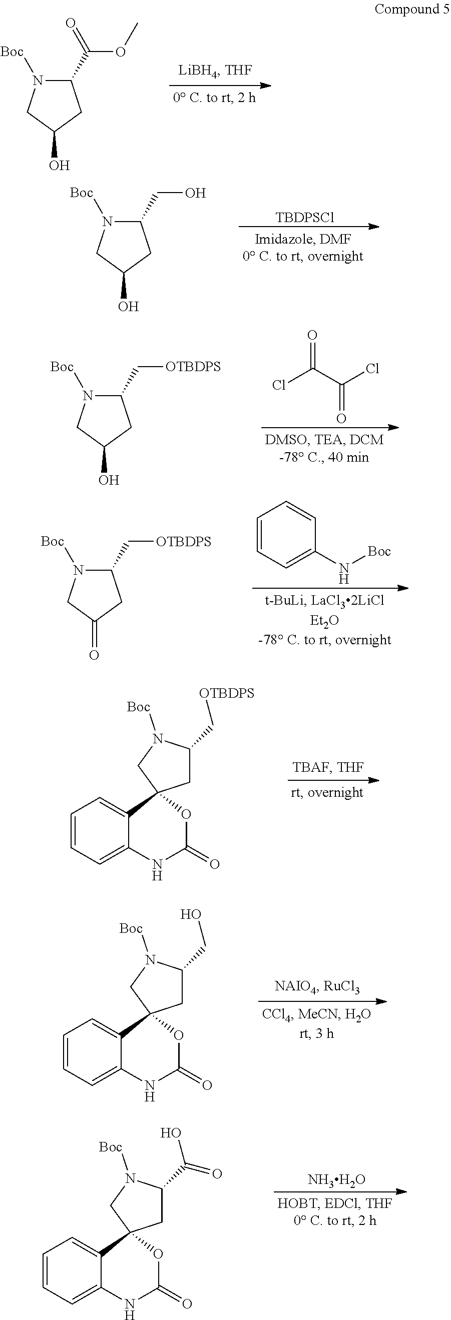

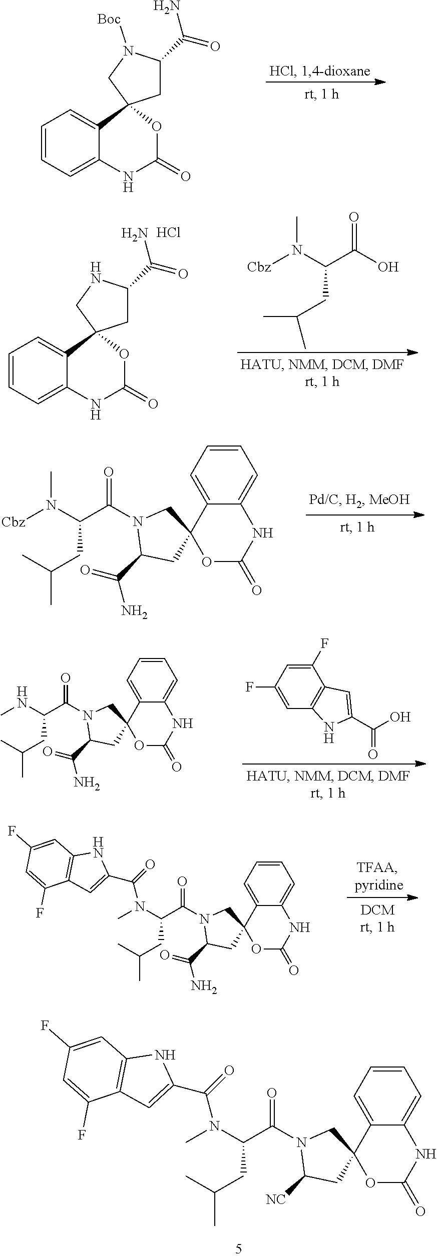

To a solution of 1-(t-butyl) 2-methyl (2S,4R)-4-hydroxypyrrolidine-1,2-dicarboxylate (20.0 g, 81.5 mmol) in THF (200 mL) was added lithium borohydride (81.5 mL, 163 mmol, 2 M in THF) at 0° C. The mixture was stirred for 2 h rt. The reaction was quenched with ice water (500 mL). The mixture was extracted with EA (3×500 mL). The organic layers were combined, washed with brine (2×300 mL) and dried over anhydrous Na₂SO₄. The solids were removed by filtration and the filtrate was concentrated under reduced pressure to afford t-butyl (2S,4R)-4-hydroxy-2-(hydroxymethyl)pyrrolidine-1-carboxylate (16.0 g, 90%) as a light yellow solid. LC-MS (ESI, m/z): 218 [M+H]⁺.

To a solution of t-butyl (2S,4R)-4-hydroxy-2-(hydroxymethyl)pyrrolidine-1-carboxylate (10.0 g, 46.1 mmol) and imidazole (6.27 g, 92.1 mmol) in DMF (150 mL) was added t-butyldiphenylchlorosilane (13.9 g, 50.6 mmol) at 0° C. The mixture was stirred overnight at rt. The reaction was quenched with water (200 mL). The mixture was extracted with EA (3×400 mL). The organic layers were combined, washed with brine (3×400 mL) and dried over anhydrous Na₂SO₄. The solids were removed by filtration and the filtrate was concentrated under reduced pressure to afford the crude product that was chromatographed on a silica gel column with EA:PE (30%-40%) to provide t-butyl (2S,4R)-2-(((t-butyldiphenylsilyl)oxy)methyl)-4-hydroxypyrrolidine-1-carboxylate (14.0 g, 66%) as a yellow solid. LC-MS (ESI, m/z): 456 [M+H]⁺.

To a stirred solution of oxalyl dichloride (6.68 g, 52.7 mmol) in dry DCM (150 mL) was added DMSO (6.16 g, 78.9 mmol) dropwise at −78° C. under nitrogen. After stirred for 5 min, t-butyl (2S,4R)-2-(((t-butyldiphenylsilyl)oxy)methyl)-4-hydroxypyrrolidine-1-carboxylate (12.0 g, 26.3 mmol in 50 mL DCM) was added. The mixture was stirred for 15 min at −78° C. Triethylamine (15.9 g, 158 mmol) was added, and the mixture was stirred for another 20 min −78° C. The reaction was quenched with water (200 mL) and the mixture was warmed to rt. The mixture was extracted with DCM (3×300 mL). The organic layers were combined, washed with brine (2×300 mL) and dried over anhydrous Na₂SO₄. The solids were removed by filtration and the filtrate was concentrated under reduced pressure to afford the crude product that was chromatographed on a silica gel column with EA:PE (18%-22%) to provide t-butyl (S)-2-(((t-butyldiphenylsilyl)oxy)methyl)-4-oxopyrrolidine-1-carboxylate (8.0 g, 67%) as a white solid. LC-MS (ESI, m/z): 454 [M+H]⁺.

To a stirred mixture of t-butyl phenylcarbamate (893 mg, 4.63 mmol) in Et₂O (21 mL) was added t-butyllithium (8.30 mL, 10.8 mmol, 1.3 M in n-pentane) at −40° C. under nitrogen. The mixture was stirred for 4 h at below −10° C. Lanthanum(III) chloride bis(lithium chloride) complex solution (10.3 mL, 6.16 mmol, 0.6 M in THF) was added at −78° C. After stirred 5 min, t-butyl (S)-2-(((t-butyldiphenylsilyl)oxy)methyl)-4-oxopyrrolidine-1-carboxylate (700 mg, 1.54 mmol in 15 mL Et₂O) was added −78° C. The mixture was stirred overnight at rt. Potassium t-butoxide (17 mg, 0.154 mmol) and THF (20 mL) were added. The mixture was stirred for 4 h at 50° C. The reaction was quenched with H₂O (100 mL) and the mixture was extracted with EA (3×150 mL). The organic layers were combined, washed with brine (2×100 mL) and dried over anhydrous Na₂SO₄. The solids were removed by filtration and the filtrate was concentrated under reduced pressure to afford the crude product that was chromatographed on a silica to afford t-butyl (4R,5'S)-5′-(((t-butyldiphenylsilyl)oxy)methyl)-2-oxo-1,2-dihydrospiro[benzo[d][1,3]oxazine-4,3′-pyrrolidine]-1′-carboxylate (400 mg, 45%) as a yellow solid. LC-MS (ESI, m/z): 573 [M+H]⁺.

To a mixture of t-butyl (4R,5'S)-5′-(((t-butyldiphenylsilyl)oxy)methyl)-2-oxo-1,2-dihydrospiro[benzo[d][1,3]oxazine-4,3′-pyrrolidine]-1′-carboxylate (400 mg, 0.698 mmol) in THF (4 mL) was added tetrabutylammonium fluoride (1.4 mL, 1.40 mmol, 1 M in THF) at 0° C. The mixture was stirred overnight at rt. The reaction was quenched with water (50 mL). The mixture was extracted with EA (3×100 mL). The organic layers were combined, washed with 0.5 M HCl (3×50 mL) and brine (3×50 mL) and dried over anhydrous Na₂SO₄. The solids were removed by filtration and the filtrate was concentrated under reduced pressure to afford t-butyl (4R,5'S)-5′-(hydroxymethyl)-2-oxo-1,2-dihydrospiro[benzo[d][1,3]oxazine-4,3′-pyrrolidine]-1′-carboxylate (400 mg, crude) as dark yellow solid. LC-MS (ESI, m/z): 335 [M+H]⁺.

To a mixture of t-butyl (4R,5'S)-5′-(hydroxymethyl)-2-oxo-1,2-dihydrospiro[benzo[d][1,3]oxazine-4,3′-pyrrolidine]-1′-carboxylate (400 mg, 1.19 mmol) in CCl₄ (3 mL) and CH₃CN (3 mL) were added sodium periodate (1.28 g, 5.98 mmol in 4.5 mL H₂O) and trichlororuthenium (29.7 mg, 0.144 mmol). The mixture was stirred for 2 h at rt and then filtered through celite. The filtrate was diluted with water (50 mL) and extracted with DCM (3×100 mL). The organic layers were combined, washed with brine (2×50 mL) and dried over anhydrous Na₂SO₄. The solids were removed by filtration and the filtrate was concentrated under reduced pressure to afford the crude product that was purified by C18 column with CH₃CN/water (0.05% FA). The fraction (35%) was concentrated under reduced pressure to provide (4R,5'S)-1′-(t-butoxycarbonyl)-2-oxo-1,2-dihydrospiro[benzo[d][1,3]oxazine-4,3′-pyrrolidine]-5′-carboxylic acid (140 mg, crude) as an off-white solid. LC-MS (ESI, m/z): 349 [M+H]⁺.

To a mixture of (4R,5'S)-1′-(t-butoxycarbonyl)-2-oxo-1,2-dihydrospiro[benzo[d][1,3]oxazine-4,3′-pyrrolidine]-5′-carboxylic acid (140 mg, 0.402 mmol) in THF (2 mL) were added 1-hydroxybenzotriazole (163 mg, 1.21 mmol) and 1-(3-dimethylaminopropyl)-3-ethylcarbodiimide hydrochloride (169 mg, 0.884 mmol). The mixture was stirred at 0° C. After stirring for 1 h, NH₄OH (1.8 mL) was added. The mixture was stirred for 3 h at rt and then concentrated under reduced pressure to afford the crude product that was purified by C18 column with CH₃CN/water (0.05% FA). The fraction was concentrated under reduced pressure to provide t-butyl (4R,5'S)-5′-carbamoyl-2-oxo-1,2-dihydrospiro[benzo[d][1,3]oxazine-4,3′-pyrrolidine]-1′-carboxylate (100 mg, 71%) as a white solid. LC-MS (ESI, m/z): 348 [M+H]⁺.

To a mixture of t-butyl (4R,5'S)-5′-carbamoyl-2-oxo-1,2-dihydrospiro[benzo[d][1,3]oxazine-4,3′-pyrrolidine]-1′-carboxylate (100 mg, 0.288 mmol) in 1,4-dioxane (1 mL) was added hydrogen chloride (2 mL, 4 M in 1,4-dioxane) at rt. The mixture was stirred for 1 h and then concentrated under reduced pressure to afford (4R,5'S)-2-oxo-1,2-dihydrospiro[benzo[d][1,3]oxazine-4,3′-pyrrolidine]-5′-carboxamide hydrochloride (80 mg, crude) as a light yellow solid. LC-MS (ESI, m/z): 248 [M+H]⁺.

To a solution of (4R,5'S)-2-oxo-1,2-dihydrospiro[benzo[d][1,3]oxazine-4,3′-pyrrolidine]-5′-carboxamide hydrochloride (80.0 mg, 0.282 mmol), (2S)-2-{[(benzyloxy)carbonyl](methyl)amino}-4-methylpentanoic acid (78.8 mg, 0.282 mmol) and HATU (118 mg, 0.310 mmol) in DCM (1.8 mL) and DMF (0.3 mL) was added 4-methylmorpholine (85.6 mg, 0.846 mmol) at 0° C. The mixture was stirred for 1 h at rt. The reaction was quenched with water (20 mL). The mixture was extracted with EA (3×60 mL). The organic layers were combined, washed with brine (2×30 mL) and dried over anhydrous Na₂SO₄. The solids were removed by filtration and the filtrate was concentrated under reduced pressure to afford the crude product that was purified by TLC (Mobile phase: EA:PE=5:1; Rf=0.4; detection: UV) and then further purified by C18 column with CH₃CN/water (0.05% FA). The fraction was concentrated under reduced pressure to provide benzyl ((S)-1-((4R,5'S)-5′-carbamoyl-2-oxo-1,2-dihydrospiro[benzo[d][1,3]oxazine-4,3′-pyrrolidin]-1′-yl)-4-methyl-1-oxopentan-2-yl)(methyl)carbamate (60 mg, 41%) as an off-white solid. LC-MS (ESI, m/z): 509 [M+H]⁺.

To a mixture of benzyl ((S)-1-((4R,5'S)-5′-carbamoyl-2-oxo-1,2-dihydrospiro[benzo[d][1,3]oxazine-4,3′-pyrrolidin]-1′-yl)-4-methyl-1-oxopentan-2-yl)(methyl)carbamate (60.0 mg, 0.118 mmol) in MeOH (3 mL) was added 10% Pd on activated carbon (15 mg) at rt under hydrogen. The mixture was stirred for 1 h at rt. The mixture was filtered through a celite pad and washed with MeOH (3×50 mL). The filtrate was concentrated under reduced pressure to afford (4R,5'S)-1′-(methyl-L-leucyl)-2-oxo-1,2-dihydrospiro[benzo[d][1,3]oxazine-4,3′-pyrrolidine]-5′-carboxamide (40 mg, crude) as an off-white solid. LC-MS (ESI, m/z): 375 [M+H]⁺.

To a solution of (4R,5'S)-1′-(methyl-L-leucyl)-2-oxo-1,2-dihydrospiro[benzo[d][1,3]oxazine-4,3′-pyrrolidine]-5′-carboxamide (40.0 mg, 0.107 mmol), 4,6-difluoro-1H-indole-2-carboxylic acid (21.1 mg, 0.107 mmol) and HATU (44.7 mg, 0.118 mmol) in DCM (1.2 mL) and DMF (0.2 mL) was added 4-methylmorpholine (32.4 mg, 0.321 mmol). The mixture was stirred at 0° C. The mixture was then stirred for 1 h at rt. The reaction was quenched with water (10 mL). The mixture was extracted with EA (3×30 mL). The organic layers were combined, washed with brine (2×20 mL) and dried over anhydrous Na₂SO₄. The solids were removed by filtration and the filtrate was concentrated under reduced pressure to afford the crude product that was purified by TLC (Mobile phase: EA; Rf=0.3; detection: UV) to provide (4R,5'S)-1′-(N-(4,6-difluoro-1H-indole-2-carbonyl)-N-methyl-L-leucyl)-2-oxo-1,2-dihydrospiro[benzo[d][1,3]oxazine-4,3′-pyrrolidine]-5′-carboxamide (35 mg, 59%) as a light yellow solid. LC-MS (ESI, m/z): 554 [M+H]⁺.

To a solution of (4R,5'S)-1′-(N-(4,6-difluoro-1H-indole-2-carbonyl)-N-methyl-L-leucyl)-2-oxo-1,2-dihydrospiro[benzo[d][1,3]oxazine-4,3′-pyrrolidine]-5′-carboxamide (35.0 mg, 0.063 mmol) in DCM (1 mL) were added pyridine (25.0 mg, 0.316 mmol) and trifluoroacetic anhydride (23.8 mg, 0.113 mmol). The mixture was stirred 1 h at rt. The reaction was quenched with water (10 mL). The mixture was extracted with DCM (3×20 mL). The organic layers were combined, washed with brine (2×20 mL) and dried over anhydrous Na₂SO₄. The solids were removed by filtration and the filtrate was concentrated under reduced pressure to afford the crude product that was purified by prep-HPLC (Column: Xselect CSH Prep C18 OBD Column, 19×250 mm, 5 m; Mobile Phase A: water (0.1% FA), Mobile Phase B: ACN; Flow rate: 25 mL/min; Gradient: 46% B to 76% B in 7 min, 76% B; Wave Length: 254 nm; RT1 (min): 6.78;) to provide N—((S)-1-((4R,5'S)-5′-cyano-2-oxo-1,2-dihydrospiro[benzo[d][1,3]oxazine-4,3′-pyrrolidin]-1′-yl)-4-methyl-1-oxopentan-2-yl)-4,6-difluoro-N-methyl-1H-indole-2-carboxamide (8.5 mg, 25%) as a white solid. ¹H NMR (400 MHz, 100° C., DMSO-d₆) δ 11.62-11.90 (m, 1H), 10.01-10.33 (m, 1H), 7.20-7.35 (m, 2H), 6.90-7.10 (m, 4H), 6.82-6.90 (m, 1H), 5.15-5.55 (m, 2H), 4.10-4.20 (m, 1H), 3.82-3.90 (m, 1H), 3.25 (s, 3H), 2.99-3.04 (m, 1H), 2.65-2.75 (m, 1H), 1.75-1.80 (m, 2H), 0.52-0.65 (m, 1H), 0.82-1.05 (m, 6H). LC-MS (ESI, m/z): 553 [M+NH₃]⁺.

### Example 6

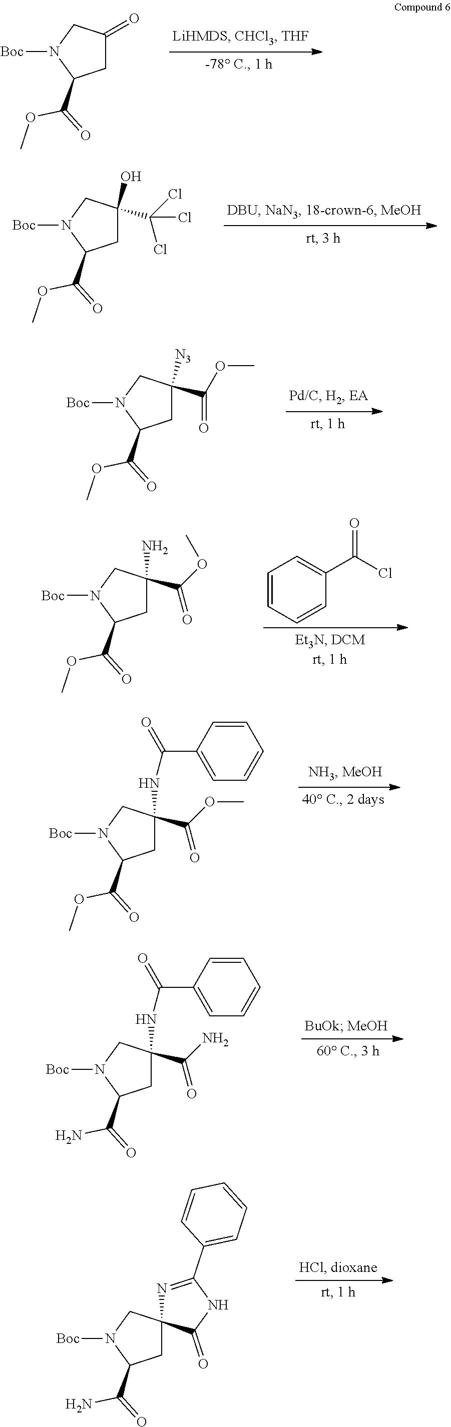

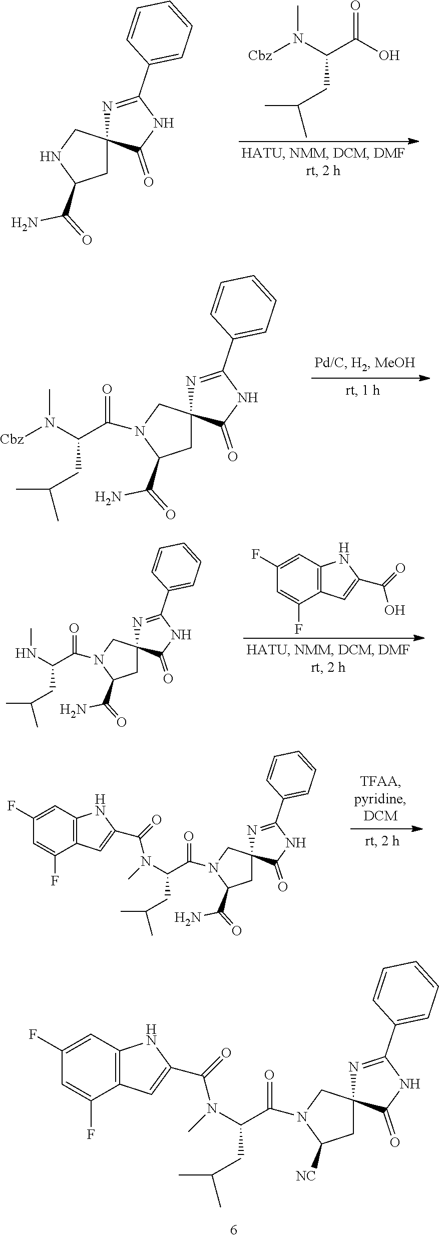

To a mixture of 1-t-butyl 2-methyl (2S)-4-oxopyrrolidine-1,2-dicarboxylate (10.0 g, 41.1 mmol) and chloroform (10.0 g, 82.2 mmol) in THF (100 mL) was added lithium bis(trimethylsilyl)amide (14.0 g, 82.2 mmol) at −78° C. under nitrogen. The mixture was stirred for 1 h at −78° C. The reaction was quenched with NH₄Cl (sat. aq. 100 mL). The mixture was extracted with EA (3×100 mL). The organic layers were combined, washed with brine (2×100 mL), dried over anhydrous Na₂SO₄, filtered and concentrated under reduced pressure to afford the crude product that was chromatographed on a silica gel column with EA:PE (1:5) to afford 1-(t-butyl) 2-methyl (2S,4R)-4-hydroxy-4-(trichloromethyl)pyrrolidine-1,2-dicarboxylate (5.20 g, crude) as a light yellow solid. LC-MS (ESI, m/z): 362 [M+H]⁺.

To a mixture of 1-(t-butyl) 2-methyl (2R,4S)-4-hydroxy-4-(trichloromethyl)pyrrolidine-1,2-dicarboxylate (5.00 g, 13.8 mmol), sodium azide (2.70 g, 41.4 mmol) and 18-crown-6 (0.05 g, 0.207 mmol) in MeOH (500 mL) was added 1,8-diazabicyclo[5.4.0]undec-7-ene (11.0 g, 68.9 mmol) under nitrogen. The mixture was stirred for 3 h at rt and then concentrated under reduced pressure to remove the MeOH. The residue was chromatographed on a silica gel column with EA:PE (1:3) to afford 1-(t-butyl) 2,4-dimethyl (2S,4R)-4-azidopyrrolidine-1,2,4-tricarboxylate (2.60 g, crude) as a yellow oil. LC-MS (ESI, m/z): 329 [M+H]⁺.

To a stirred mixture of 1-(t-butyl) 2,4-dimethyl (2S,4R)-4-azidopyrrolidine-1,2,4-tricarboxylate (1.40 g, 4.26 mmol) in EA (20 mL) was added 10% Pd/C (500 mg). The mixture was stirred for 1 h at rt under hydrogen. The mixture was filtered through a celite pad and washed with EA (3×30 mL). The filtrate was concentrated under reduced pressure to afford 1-(t-butyl) 2,4-dimethyl (2S,4R)-4-aminopyrrolidine-1,2,4-tricarboxylate (1.3 g, crude) as an off-white solid. LC-MS (ESI, m/z): 303 [M+H]⁺.

To a stirred mixture of 1-(t-butyl) 2,4-dimethyl (2S,4R)-4-aminopyrrolidine-1,2,4-tricarboxylate (1.30 g, 4.30 mmol) in DCM (15 mL) were added triethylamine (1.09 g, 10.7 mmol) and benzoyl chloride (0.660 g, 4.73 mmol). The mixture was stirred for 1 h at rt. The reaction was quenched with water (80 mL). The mixture was extracted with DCM (3×80 mL). The organic layers were combined, washed with brine (2×80 mL) and dried over anhydrous Na₂SO₄. The solids were removed by filtration and the filtrate was concentrated under reduced pressure to afford 1-(t-butyl) 2,4-dimethyl (2S,4R)-4-benzamidopyrrolidine-1,2,4-tricarboxylate (1.4 g, 80%, crude) as a brown oil. ¹H NMR (400 MHz, DMSO-d₆) δ 8.98-9.12 (m, 1H), 7.80-7.90 (m, 2H), 7.44-7.63 (m, 3H), 4.36-4.53 (m, 1H), 3.97-4.06 (m, 1H), 3.73-3.83 (m, 1H), 3.61-3.72 (m, 6H), 2.68-2.72 (m, 1H), 2.30-2.43 (m, 1H), 1.28-1.37 (m, 9H). LC-MS (ESI, m/z): 407 [M+H]⁺.

1-(t-butyl) 2,4-dimethyl (2S,4R)-4-benzamidopyrrolidine-1,2,4-tricarboxylate (1.50 g, 3.69 mmol) was stirred for 2 d at 40° C. in NH₃ (20 mL, 7M in MeOH) in a sealed vessel. The mixture was concentrated under reduced pressure to afford t-butyl (2S,4R)-4-benzamido-2,4-dicarbamoylpyrrolidine-1-carboxylate (1.4 g, crude) as a brown oil. LC-MS (ESI, m/z): 377 [M+H]⁺.

To a stirred mixture of t-butyl (2S,4R)-4-benzamido-2,4-dicarbamoylpyrrolidine-1-carboxylate (700 mg, 1.86 mmol) in MeOH (10 mL) was added potassium 2-methylpropan-2-olate (626 mg, 5.58 mmol). The mixture was stirred for 3 h at 60° C. The mixture was purified by C18 column with CH₃CN/Water (0.05% TFA). The fraction was concentrated under reduced pressure to provide t-butyl (5R,8S)-8-carbamoyl-4-oxo-2-phenyl-1,3,7-triazaspiro[4.4]non-1-ene-7-carboxylate (350 mg, 49%) as a yellow semi-solid. LC-MS (ESI, m/z): 359 [M+H]⁺.

To a stirred mixture of t-butyl (5R,8S)-8-carbamoyl-4-oxo-2-phenyl-1,3,7-triazaspiro[4.4]non-1-ene-7-carboxylate (100 mg, 0.279 mmol) in 1,4-dioxane (0.5 mL) was added hydrogen chloride (2 mL, 4 mL in 1,4-dioxane). The mixture was stirred for 1 h at rt and then concentrated under reduced pressure to afford (5R,8S)-4-oxo-2-phenyl-1,3,7-triazaspiro[4.4]non-1-ene-8-carboxamide (72 mg, crude) as an off-white solid. LC-MS (ESI, m/z): 259 [M+H]⁺.

To a stirred mixture of (5R,8S)-4-oxo-2-phenyl-1,3,7-triazaspiro[4.4]non-1-ene-8-carboxamide (72.0 mg, 0.279 mmol) and HATU (109 mg, 0.287 mmol) in DCM (1 mL) and DMF (0.3 mL) was added N-methylmorpholine (84.6 mg, 0.837 mmol). The mixture was stirred for 2 h at rt. The reaction was quenched with water (20 mL). The mixture was extracted with EA (3×20 mL). The organic layers were combined, washed with brine (3×20 mL) and dried over anhydrous Na₂SO₄. The solids were removed by filtration and the filtrate was concentrated under reduced pressure to afford the crude product that was purified by TLC (Mobile phase: EA; Rf=0.3; detection: UV) to provide benzyl N-[(2S)-1-[(5R,8S)-8-carbamoyl-4-oxo-2-phenyl-1,3,7-triazaspiro[4.4]non-1-en-7-yl]-4-methyl-1-oxopentan-2-yl]-N-methylcarbamate (100 mg, crude) as an off-white solid. LC-MS (ESI, m/z): 520 [M+H]⁺.

To a stirred mixture of benzyl N-[(2S)-1-[(5R,8S)-8-carbamoyl-4-oxo-2-phenyl-1,3,7-triazaspiro[4.4]non-1-en-7-yl]-4-methyl-1-oxopentan-2-yl]-N-methylcarbamate (100 mg, 0.192 mmol) in MeOH (3 mL) was added 10% Pd/C (40 mg). The mixture was stirred for 1 h at rt under hydrogen. The mixture was filtered through a celite pad and washed with MeOH (3×30 mL). The filtrate was concentrated under reduced pressure to afford (5R,8S)-7-[(2S)-4-methyl-2-(methylamino)pentanoyl]-4-oxo-2-phenyl-1,3,7-triazaspiro[4.4]non-1-ene-8-carboxamide (74 mg, crude) as an off-white solid. LC-MS (ESI, m/z): 386 [M+H]⁺.

To a stirred mixture of (5R,8S)-7-[(2S)-4-methyl-2-(methylamino)pentanoyl]-4-oxo-2-phenyl-1,3,7-triazaspiro[4.4]non-1-ene-8-carboxamide (74.0 mg, 0.192 mmol) and HATU (76.6 mg, 0.202 mmol) in DCM (2 mL) and DMF (0.5 mL) was added N-methylmorpholine (58.2 mg, 0.576 mmol). The mixture was stirred for 2 h at rt. The reaction was quenched with water (20 mL). The mixture was extracted with EA (3×20 mL). The organic layers were combined, washed with brine (3×20 mL) and dried over anhydrous Na₂SO₄. The solids were removed by filtration and the filtrate was concentrated under reduced pressure to afford the crude product that was chromatographed on a silica gel column with MeOH:DCM (6:100) to provide (5R,8S)-7-[(2S)-2-[1-(4,6-difluoro-1H-indol-2-yl)-N-methylformamido]-4-methylpentanoyl]-4-oxo-2-phenyl-1,3,7-triazaspiro[4.4]non-1-ene-8-carboxamide (56 mg, crude) as an off-white solid. LC-MS (ESI, m/z): 565 [M+H]⁺.

To a stirred mixture of (5R,8S)-7-[(2S)-2-[1-(4,6-difluoro-1H-indol-2-yl)-N-methylformamido]-4-methylpentanoyl]-4-oxo-2-phenyl-1,3,7-triazaspiro[4.4]non-1-ene-8-carboxamide (50.0 mg, 0.089 mmol) in DCM (1 mL) were added pyridine (28.0 mg, 0.356 mmol) and trifluoroacetic anhydride (27.9 mg, 0.134 mmol). The mixture was stirred for 2 h at rt. The reaction was quenched with water (20 mL). The mixture was extracted with DCM (3×20 mL). The organic layers were combined, washed with brine (2×20 mL) and dried over anhydrous Na₂SO₄. The solids were removed by filtration and the filtrate was concentrated under reduced pressure to afford the crude product that was purified by prep-HPLC (Column: Xselect CSH Prep C18 OBD Column, 19×250 mm, 5 m; Mobile Phase A: water (0.1% FA), Mobile Phase B: ACN; Flow rate: 25 mL/min; Gradient: 48% B to 78% B in 7 min, 78% B; Wave Length: 254 nm; RT1 (min): 5.78) to provide N-[(2S)-1-[(5R,8S)-8-cyano-4-oxo-2-phenyl-1,3,7-triazaspiro[4.4]non-1-en-7-yl]-4-methyl-1-oxopentan-2-yl]-4,6-difluoro-N-methyl-1H-indole-2-carboxamide (12.2 mg, 25%) as a white solid. ¹H NMR (400 MHz, 100° C., DMSO-d₆) δ 11.50 (br, 2H), 7.70-8.10 (m, 2H), 7.20-7.69 (m, 3H), 6.60-7.19 (m, 3H), 5.25-5.70 (m, 1H), 4.90-5.24 (m, 1H), 3.60-4.30 (m, 2H), 3.20-3.30 (m, 3H), 2.53-2.78 (m, 1H), 2.41-2.48 (m, 1H), 1.70-1.90 (m, 2H), 1.45-1.69 (m, 1H), 0.90-1.00 (m, 6H). LC-MS (ESI, m/z): 547 [M+H]⁺.

### Example 7

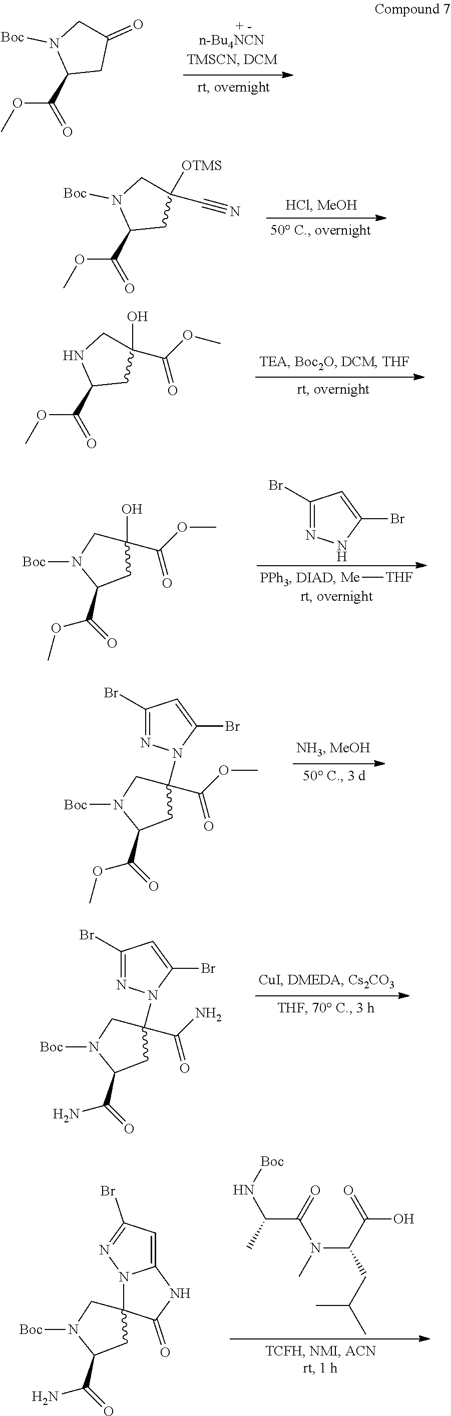

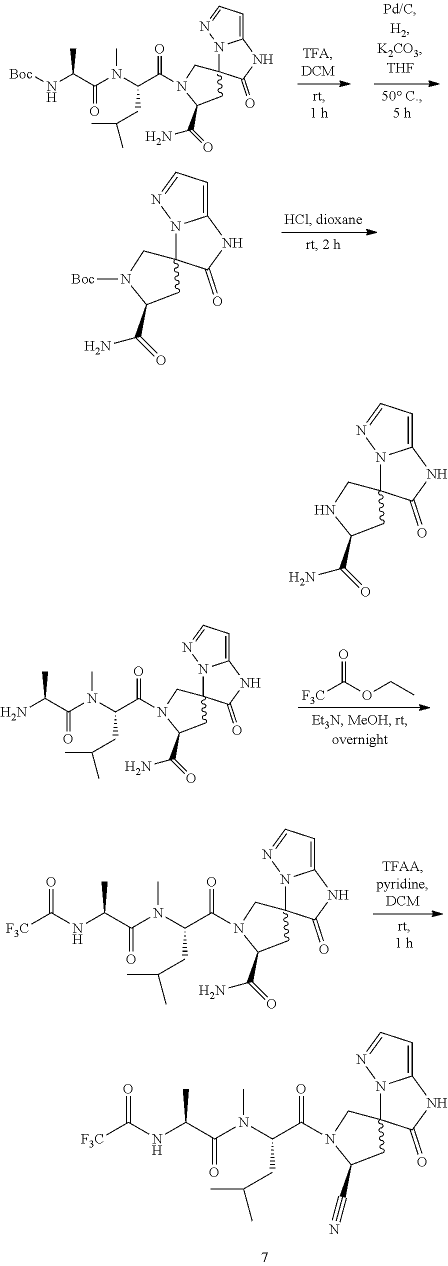

To a mixture of 1-t-butyl 2-methyl (2S)-4-oxopyrrolidine-1,2-dicarboxylate (10.0 g, 41.1 mmol) in DCM (100 mL) was added trimethylsilyl cyanide (8.2 g, 82.2 mmol) and tetrabutylammonium cyanide (1.2 g, 4.11 mmol) under nitrogen. The mixture was stirred overnight at rt. The reaction was quenched with water (100 mL). The mixture was extracted with DCM (3×100 mL). The organic layers were combined, washed with brine (2×100 mL) and dried over anhydrous Na₂SO₄. The solids were removed by filtration and the filtrate was concentrated under reduced pressure to afford 1-t-butyl 2-methyl (2S)-4-cyano-4-[(trimethylsilyl)oxy]pyrrolidine-1,2-dicarboxylate (14.0 g, crude) as a brown oil. LC-MS (ESI, m/z): 287 [M−56+H]⁺.

A mixture of 1-t-butyl 2-methyl (2S)-4-cyano-4-hydroxypyrrolidine-1,2-dicarboxylate (14.0 g, 0.052 mmol) in HCl (140 mL, 4 M in MeOH). The mixture was stirred overnight at 50° C. and concentrated under reduced pressure to afford 2,4-dimethyl (2S)-4-hydroxypyrrolidine-2,4-dicarboxylate (8.4 g, crude) as a black oil. LC-MS (ESI, m/z): 204 [M+H]⁺.

To a mixture of 2,4-dimethyl (2S)-4-hydroxypyrrolidine-2,4-dicarboxylate (8.4 g, 41.3 mmol) in DCM (85 mL) and THF (35 mL) were added trimethylamine (17.0 g, 165 mmol) and di-t-butyl dicarbonate (18.0 g, 82.7 mmol). The mixture was stirred overnight at rt. The reaction was quenched with water (100 mL). The mixture was extracted with EA (3×100 mL). The organic layers were combined, washed with brine (2×100 mL) and dried over anhydrous Na₂SO₄. The solids were removed by filtration and the filtrate was concentrated under reduced pressure to afford the crude product that was chromatographed on a silica gel column with EA:PE (1:4) to provide 1-t-butyl 2,4-dimethyl (2S)-4-hydroxypyrrolidine-1,2,4-tricarboxylate (6.2 g, 42%) as a yellow oil. LC-MS (ESI, m/z): 204 [M−Boc+H]⁺.

To a mixture of 3,5-dibromo-1H-pyrazole (1.12 g, 4.97 mmol), 1-t-butyl 2,4-dimethyl (2S)-4-hydroxypyrrolidine-1,2,4-tricarboxylate (1.00 g, 3.30 mmol) and triphenylphosphine (2.59 g, 9.89 mmol) in 2-methyl-THF (20 mL) was added diisopropyl azodicarboxylate (2.00 g, 9.89 mmol) at 0° C. The mixture was stirred overnight at rt. The reaction was quenched with water (50 mL). The mixture was extracted with EA (3×50 mL). The organic layers were combined, washed with brine (2×50 mL), dried over anhydrous Na₂SO₄. The solids were removed by filtration and the filtrate was concentrated under reduced pressure to afford the crude product that was chromatographed on a silica gel column with EA:PE (1:3) and then further purified by C18 column with CH₃CN/water (0.05% TFA). The fraction was concentrated under reduced pressure to provide 1-t-butyl 2,4-dimethyl (2S)-4-(3,5-dibromopyrazol-1-yl)pyrrolidine-1,2,4-tricarboxylate (600 mg, 30%) as a yellow oil. LC-MS (ESI, m/z): 454 [M−56+H]⁺.

A mixture of 1-t-butyl 2,4-dimethyl (2S)-4-(3,5-dibromopyrazol-1-yl)pyrrolidine-1,2,4-tricarboxylate (600 mg, 1.17 mmol) in ammonia (20 mL, 7 M in MeOH) was stirred for 3 days at 50° C. The mixture was concentrated under reduced pressure to afford t-butyl (2S)-2,4-dicarbamoyl-4-(3,5-dibromopyrazol-1-yl)pyrrolidine-1-carboxylate (565 mg, crude) as an off-white solid. LC-MS (ESI, m/z): 380 [M−Boc+H]⁺.

To a mixture of t-butyl (2S)-2,4-dicarbamoyl-4-(3,5-dibromopyrazol-1-yl)pyrrolidine-1-carboxylate (565 mg, 1.17 mmol), cuprous iodide (22.0 mg, 0.117 mmol) and cesium carbonate (765 mg, 2.35 mmol) in THF (10 mL) was added N,N′-dimethyl-1,2-ethanediamine (21.0 mg, 0.235 mmol). The mixture was stirred for 3 h at 70° C. under nitrogen. The mixture was concentrated under reduced pressure to remove the solvent. The residue was chromatographed on a silica gel column with MeOH:DCM (5:95) to provide t-butyl (5'S)-6-bromo-5′-carbamoyl-2-oxo-1H-spiro[pyrazolo[1,5-a]imidazole-3,3′-pyrrolidine]-1′-carboxylate (300 mg, 59%) as an off-white solid. LC-MS (ESI, m/z): 400 [M+H]⁺.

To a mixture of t-butyl (5'S)-6-bromo-5′-carbamoyl-2-oxo-1H-spiro[pyrazolo[1,5-a]imidazole-3,3′-pyrrolidine]-1′-carboxylate (300 mg, 0.750 mmol) in THF (5 mL) were added K₂CO₃ (157 mg, 1.12 mmol) and 10% Pd/C (150 mg). The mixture was stirred for 5 h at 50° C. under hydrogen. The mixture was chromatographed on a silica gel column with MeOH:DCM (4:94) to provide t-butyl (5'S)-5′-carbamoyl-2-oxo-1H-spiro[pyrazolo[1,5-a]imidazole-3,3′-pyrrolidine]-1′-carboxylate (170 mg, 64%) as an off-white solid. LC-MS (ESI, m/z): 322 [M+H]⁺.

A mixture of t-butyl (5'S)-5′-carbamoyl-2-oxo-1H-spiro[pyrazolo[1,5-a]imidazole-3,3′-pyrrolidine]-1′-carboxylate (170 mg, 0.529 mmol) in HCl (3 mL, 4 M in dioxane) was stirred for 2 h at rt. The mixture was concentrated under reduced pressure to afford (5'S)-2-oxo-1H-spiro[pyrazolo[1,5-a]imidazole-3,3′-pyrrolidine]-5′-carboxamide (120 mg, crude) as an off-white solid. LC-MS (ESI, m/z): 222 [M+H]⁺.

To a mixture of (5'S)-2-oxo-1H-spiro[pyrazolo[1,5-a]imidazole-3,3′-pyrrolidine]-5′-carboxamide (100 mg, 0.452 mmol), (2S)-2-[(2S)-2-[(t-butoxycarbonyl)amino]-N-methylpropanamido]-4-methylpentanoic acid (143 mg, 0.452 mmol) and N,N,N′,N′-tetramethylchloroformamidinium hexafluorophosphate (TCFH) (165 mg, 0.588 mmol) in CH₃CN (3 mL) was added N-methylimidazole (371 mg, 4.52 mmol). The mixture was stirred for 1 h at rt and then purified by C18 column with CH₃CN/water (0.05% FA). The fraction was concentrated under reduced pressure to provide t-butyl N-[(1S)-1-{[(2S)-1-[(5'S)-5′-carbamoyl-2-oxo-1H-spiro[pyrazolo[1,5-a]imidazole-3,3′-pyrrolidin]-1′-yl]-4-methyl-1-oxopentan-2-yl](methyl)carbamoyl}ethyl]carbamate (117 mg, 46%) as a light yellow solid. LC-MS (ESI, m/z): 520 [M+H]⁺.

To a mixture of t-butyl N-[(1S)-1-{1[(2S)-1-[(5'S)-5′-carbamoyl-2-oxo-1H-spiro[pyrazolo[1,5-a]imidazole-3,3′-pyrrolidin]-1′-yl]-4-methyl-1-oxopentan-2-yl](methyl)carbamoyl}ethyl]carbamate (117 mg, 0.225 mmol) in DCM (1.5 mL) was added trifluoroacetic acid (0.5 mL). The mixture was stirred for 1 h at rt and then concentrated under reduced pressure to afford (5'S)-1′-[(2S)-2-[(2S)-2-amino-N-methylpropanamido]-4-methylpentanoyl]-2-oxo-1H-spiro[pyrazolo[1,5-a]imidazole-3,3′-pyrrolidine]-5′-carboxamide (94 mg, crude) as a brown oil. LC-MS (ESI, m/z): 420 [M+H]⁺.

To a mixture of (5'S)-1′-[(2S)-2-[(2S)-2-amino-N-methylpropanamido]-4-methylpentanoyl]-2-oxo-1H-spiro[pyrazolo[1,5-a]imidazole-3,3′-pyrrolidine]-5′-carboxamide (94.0 mg, 0.224 mmol) in MeOH (2 mL) were added triethylamine (272 mg, 2.69 mmol) and ethyl 2,2,2-trifluoroacetate (318 mg, 2.24 mmol). The mixture was stirred overnight at rt and then concentrated under reduced pressure to afford the crude product that was chromatographed on a silica gel column with MeOH:DCM (5/95) to provide (5'S)-1′-[(2S)-4-methyl-2-[(2S)—N-methyl-2-(2,2,2-trifluoroacetamido)propanamido]pentanoyl]-2-oxo-1H-spiro[pyrazolo[1,5-a]imidazole-3,3′-pyrrolidine]-5′-carboxamide (110 mg, crude) as a yellow oil. LC-MS (ESI, m/z): 516 [M+H]⁺.

To a stirred mixture of (5'S)-1′-[(2S)-4-methyl-2-[(2S)—N-methyl-2-(2,2,2-trifluoroacetamido)propanamido]pentanoyl]-2-oxo-1H-spiro[pyrazolo[1,5-a]imidazole-3,3′-pyrrolidine]-5′-carboxamide (100 mg, 0.194 mmol) in DCM (2 mL) were added pyridine (61.0 mg, 0.776 mmol) and trifluoroacetic anhydride (81.0 mg, 0.388 mmol). The mixture was stirred for 1 h at rt. The reaction was quenched with water (5 mL). The mixture was extracted with DCM (3×5 mL). The organic layers were combined, washed with brine (2×5 mL), dried over anhydrous Na₂SO₄. The solids were removed by filtration and the filtrate was concentrated under reduced pressure to afford the crude product that was purified by prep-HPLC (Column: Xselect CSH C18 OBD Column 30×150 mm 5 m, n; Mobile Phase A: water (0.1% FA), Mobile Phase B: ACN; Flow rate: 60 mL/min; Gradient: 30% B to 60% B in 8 min; Wave Length: 254; 220 nm; RT1 (min): 6.27) to provide (2S)—N-[(2S)-1-[(5'S)-5′-cyano-2-oxo-1H-spiro[pyrazolo[1,5-a]imidazole-3,3′-pyrrolidin]-1′-yl]-4-methyl-1-oxopentan-2-yl]-N-methyl-2-(2,2,2-trifluoroacetamido)propanamide (38.3 mg, 39%) as a white solid. ¹H NMR (400 MHz, DMSO-d₆) δ 11.55 (br, 1H), 9.43-9.76 (m, 1H), 7.35-7.54 (m, 1H), 5.62-5.80 (m, 1H), 4.98-5.28 (m, 2H), 4.55-4.83 (m, 1H), 3.55-4.05 (m, 2H), 2.70-2.98 (m, 4H), 2.52-2.69 (m, 1H), 1.16-1.70 (m, 6H), 0.75-0.96 (m, 6H). LC-MS (ESI, m/z): 498 [M+H]⁺.

### Example 8

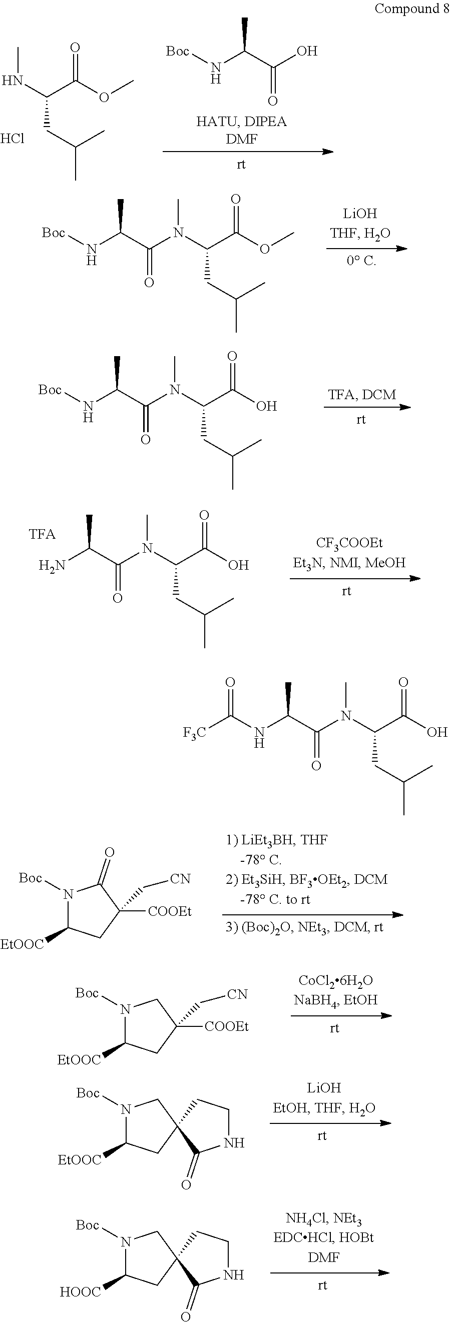

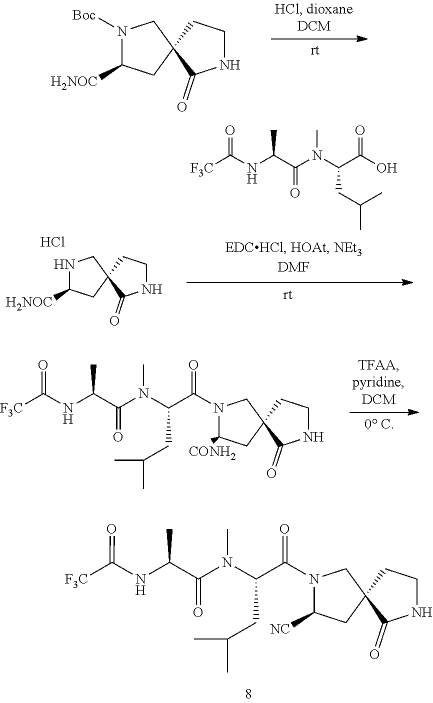 [SMILES: CC(C)(C)OC(=O)N1C[C@]2(CCNC2=O)C[C@H]1C(N)=O ; CC(C)C[C@@H](C(=O)O)N(C)C(=O)[C@H](C)NC(=O)C(F)(F)F ; Cl ; NC(=O)[C@@H]1C[C@@]2(CCNC2=O)CN1 ; CC(C)C[C@@H](C(=O)N1C[C@]2(CCNC2=O)C[C@H]1C(N)=O)N(C)C(=O)[C@H](C)NC(=O)C(F)(F)F ; CC(C)C[C@@H](C(=O)N1C[C@]2(CCNC2=O)C[C@H]1C#N)N(C)C(=O)[C@H](C)NC(=O)C(F)(F)F]

To a solution of methyl methyl-L-leucinate hydrochloride (2.2 g, 11.2 mmol) in DMF (20 mL) cooled to 0° C. were added (t-butoxycarbonyl)-L-alanine (2.13 g, 11.2 mmol), HATU (6.41 g, 16.9 mmol) and DIPEA (5.9 mL, 33.7 mmol). The mixture was stirred at rt for 3 h, then diluted with water (50 mL) and extracted with EA (3×100 mL). The organic phases were combined, washed with brine (2×20 mL), dried over Na₂SO₄. The solids were removed by filtration and the filtrate was concentrated under reduced pressure. The residue was purified by flash chromatography on silica gel using a gradient of EA (15 to 20%) in PE to afford methyl N-((t-butoxycarbonyl)-L-alanyl)-N-methyl-L-leucinate (3.2 g, 86%) as a colorless liquid.

To a solution of methyl N-((t-butoxycarbonyl)-L-alanyl)-N-methyl-L-leucinate (2.5 g, 7.57 mmol) in THF (16 mL) and water (8 mL) cooled to 0° C. was added LiOH (476 mg, 11.4 mmol). The mixture was stirred at 0° C. for 6 h. The mixture was acidified by addition of 1N HCl until pH ˜2 and then extracted with EA (5×50 mL). The organic phases were combined, dried over Na₂SO₄. The solids were removed by filtration and the filtrate was concentrated under reduced pressure to get N-((t-butoxycarbonyl)-L-alanyl)-N-methyl-L-leucine (2.2 g, 92%) as an off-white solid.

To a solution of N-((t-butoxycarbonyl)-L-alanyl)-N-methyl-L-leucine (1.3 g, 4.11 mmol) in DCM (13 mL) cooled to 0° C. was added TFA (2.2 mL, 28.8 mmol). The mixture was stirred at rt for 7 h. The mixture was concentrated under reduced pressure and co-evaporated with DCM to afford quantitatively N-(L-alanyl)-N-methyl-L-leucine under its TFA salt form as a colorless oil.

To a solution of N-(L-alanyl)-N-methyl-L-leucine trifluoroacetic acid salt (1.35 g, 4.09 mmol) in MeOH (13 mL) cooled to 0° C. were added ethyl trifluoroacetate (2.44 mL, 20.4 mmol), triethylamine (NEt₃) (2.84 mL, 20.4 mmol) and 1-methylimidazole (0.66 mL, 8.17 mmol). The mixture was stirred at rt for 18 h and then concentrated under reduced pressure. The residue was dissolved in EA (150 mL) and washed with 1M HCl (30 mL). The phases were separated. The organic phase was washed with 1M HCl (30 mL). The aqueous phases were combined and extracted with EA (50 mL). The organic phases were combined, washed with brine (20 mL), dried over Na₂SO₄. The solids were removed by filtration and the filtrate was concentrated under reduced pressure to afford N-methyl-N-((2,2,2-trifluoroacetyl)-L-alanyl)-L-leucine (1.2 g, 93%) as a colorless oil. LC-MS (ESI, m/z): 313 [M+H]⁺.

To a solution of 1-(t-butyl) 2,4-diethyl (2S,4R)-4-(cyanomethyl)-5-oxopyrrolidine-1,2,4-tricarboxylate (300 mg, 0.815 mmol) in THF (20 mL) cooled to −78° C. was added dropwise 1M lithium triethyl borohydride solution in THF (0.978 mL, 0.978 mmol). The mixture was stirred at −78° C. for 1 h. The reaction was quenched by addition of sat. NaHCO₃ (5 mL) and hydrogen peroxide (0.3 mL). The mixture extracted with EA (3×20 mL). The organic phases were combined, washed with brine (10 mL) and dried over Na₂SO₄. The solids were removed by filtration and the filtrate was concentrated under reduced pressure.

The residue (280 mg) was dissolved in DCM (5 mL) and the mixture was cooled to −78° C. Triethylsilane (0.098 ml, 0.616 mmol) and BF₃·OEt₂ (0.084 mL, 0.677 mmol) were added. The mixture was stirred at −78° C. for 30 min. Triethylsilane (0.098 ml, 0.616 mmol) and BF₃·OEt₂ (0.084 mL, 0.677 mmol) were added again. The mixture was allowed to warm to rt over 1 h and then concentrated under reduced pressure.

The residue (300 mg) was dissolved in DCM (10 mL). NEt₃ (0.500 mL, 3.59 mmol) and di-t-butyl dicarbonate (0.386 g, 1.77 mmol) were added. The mixture was stirred at rt for 2 h. The mixture was concentrated under reduced pressure. The residue was purified by flash chromatography on silica gel using a gradient of EA (25 to 30%) in PE to afford 1-(t-butyl) 2,4-diethyl (2S,4R)-4-(cyanomethyl)pyrrolidine-1,2,4-tricarboxylate (140 mg, 48% over 3 steps) as a colorless oil.

To a solution of 1-(t-butyl) 2,4-diethyl (2S,4R)-4-(cyanomethyl)pyrrolidine-1,2,4-tricarboxylate (470 mg, 1.32 mmol) in EtOH (5 mL) cooled to 0° C. were added CoCl₂·6H₂O (631 mg, 2.65 mmol) and NaBH₄ (401 mg, 10.6 mmol). The mixture was stirred at rt for 16 h. The mixture was diluted with 25% NH₄OH (5 mL) and water (10 mL) and then extracted with DCM (2×30 mL). The organic phases were combined, dried over Na₂SO₄. The solids were removed by filtration and the filtrate was concentrated under reduced pressure. The residue was purified by flash chromatography on silica gel using a gradient of MeOH (2 to 5%) in DCM to afford 2-(t-butyl) 3-ethyl (3S,5S)-6-oxo-2,7-diazaspiro[4.4]nonane-2,3-dicarboxylate (250 mg, 60%) as a colorless oil. LC-MS (ESI, m/z): 313 [M+H]⁺.

To a solution of 2-(t-butyl) 3-ethyl (3S,5S)-6-oxo-2,7-diazaspiro[4.4]nonane-2,3-dicarboxylate (240 mg, 0.769 mmol) in EtOH (1 mL), THF (1 mL) and water (1 mL) cooled to 0° C. was added LiOH (48 mg, 1.15 mmol). The mixture was stirred at rt for 4 h. The mixture was partially concentrated under reduced pressure to remove organic solvents. The residue was acidified by addition of 1N HCl until pH˜4 and extracted with 10% MeOH:DCM (3×10 mL). The organic phases were combined, dried over Na₂SO₄. The solids were removed by filtration and the filtrate was concentrated under reduced pressure to afford (3S,5S)-2-(t-butoxycarbonyl)-6-oxo-2,7-diazaspiro[4.4]nonane-3-carboxylic acid (200 mg, 90%) as an off-white solid.

To a solution of (3S,5S)-2-(t-butoxycarbonyl)-6-oxo-2,7-diazaspiro[4.4]nonane-3-carboxylic acid (240 mg, 0.845 mmol) in DMF (2.5 mL) cooled to 0° C. were added EDC·HCl (322 mg, 1.69 mmol), HOBt (114 mg, 0.845 mmol), NEt₃ (0.35 mL, 2.53 mmol) and NH₄Cl (90 mg, 1.69 mmol). The mixture was stirred at rt for 16 h. The mixture was diluted with water (5 mL) and extracted with 20% MeOH:DCM (3×20 mL). The organic phases were combined, dried over Na₂SO₄. The solids were removed by filtration and the filtrate was concentrated under reduced pressure. The residue was purified by flash chromatography on C18 using a gradient of ACN (15 to 20%) in 0.1% formic acid (FA) in water to afford t-butyl (3S,5S)-3-carbamoyl-6-oxo-2,7-diazaspiro[4.4]nonane-2-carboxylate (160 mg, 66%) as an off-white solid.

To a solution of t-butyl (3S,5S)-3-carbamoyl-6-oxo-2,7-diazaspiro[4.4]nonane-2-carboxylate (160 mg) in DCM (2 mL) cooled to 0° C. was added 4M HCl in dioxane (0.700 mL, 2.80 mmol). The mixture was stirred at rt for 3 h. The mixture was concentrated under reduced pressure to afford quantitatively (3S,5S)-6-oxo-2,7-diazaspiro[4.4]nonane-3-carboxamide hydrochloride as a white solid.

To a solution of N-methyl-N-((2,2,2-trifluoroacetyl)-L-alanyl)-L-leucine (100 mg, 0.320 mmol) in DMF (1 mL) cooled to 0° C. were added (3S,5S)-6-oxo-2,7-diazaspiro[4.4]nonane-3-carboxamide hydrochloride (84 mg, 0.384 mmol), EDC·HCl (121 mg, 0.631 mmol), HOAt (43 mg, 0.316 mmol) and NEt₃ (0.134 mL, 0.960 mmol). The mixture was stirred at rt for 16 h. The mixture was diluted with water (5 mL) and extracted with 10% MeOH:DCM (3×10 mL). The organic phases were combined, dried over Na₂SO₄. The solids were removed by filtration and the filtrate was concentrated under reduced pressure. The residue was purified by flash chromatography on C18 using a gradient of ACN (20 to 30%) in 0.1% FA in water to afford (3S,5S)-2-(N-methyl-N-((2,2,2-trifluoroacetyl)-L-alanyl)-L-leucyl)-6-oxo-2,7-diazaspiro[4.4]nonane-3-carboxamide (65 mg, 43%) as a white solid.

To a solution of (3S,5S)-2-(N-methyl-N-((2,2,2-trifluoroacetyl)-L-alanyl)-L-leucyl)-6-oxo-2,7-diazaspiro[4.4]nonane-3-carboxamide (60 mg, 0.125 mmol) in DCM (1 mL) cooled to 0° C. were added pyridine (0.022 mL, 0.275 mmol) and TFAA (0.011 mL, 0.138 mmol). The mixture was stirred at 0° C. for 1 h. The mixture was diluted with water (5 mL) and extracted with DCM (3×10 mL). The organic phases were combined, washed with brine (2×5 mL), dried over Na₂SO₄. The solids were removed by filtration and the filtrate was concentrated under reduced pressure. The residue was purified by preparative HPLC (Column: X-BRIDGE-C18 Column, 10×250 mm, 5 m; Mobile Phase A: 10 mM NH₄HCO₃ in water, Mobile Phase B: ACN; Flow rate: 8 mL/min; Gradient: 10% B to 60% B in 8 min) to afford (S)—N—((S)-1-((3S,5S)-3-cyano-6-oxo-2,7-diazaspiro[4.4]nonan-2-yl)-4-methyl-1-oxopentan-2-yl)-N-methyl-2-(2,2,2-trifluoroacetamido)propanamide (32 mg, 61%) as a white solid. ¹H NMR (500 MHz, 363K, DMSO-d₆) δ 9.23 (br. s., 1H), 7.62 (s, 1H), 5.19 (s, 1H), 4.67-4.86 (m, 2H), 3.76 (d, 1H), 3.50 (d, 1H), 3.19 (m, 2H), 2.95 (s, 3H), 2.38 (m, 1H), 2.22-2.32 (m, 1H), 1.96 (m, 1H), 1.90 (m, 1H), 1.54-1.70 (m, 2H), 1.51 (m, 1H), 1.26 (d, 3H) 0.89 (d, 3H), 0.86 (d, 3H). LC-MS (ESI, m/z): 460 [M+H]⁺.

### Example 9

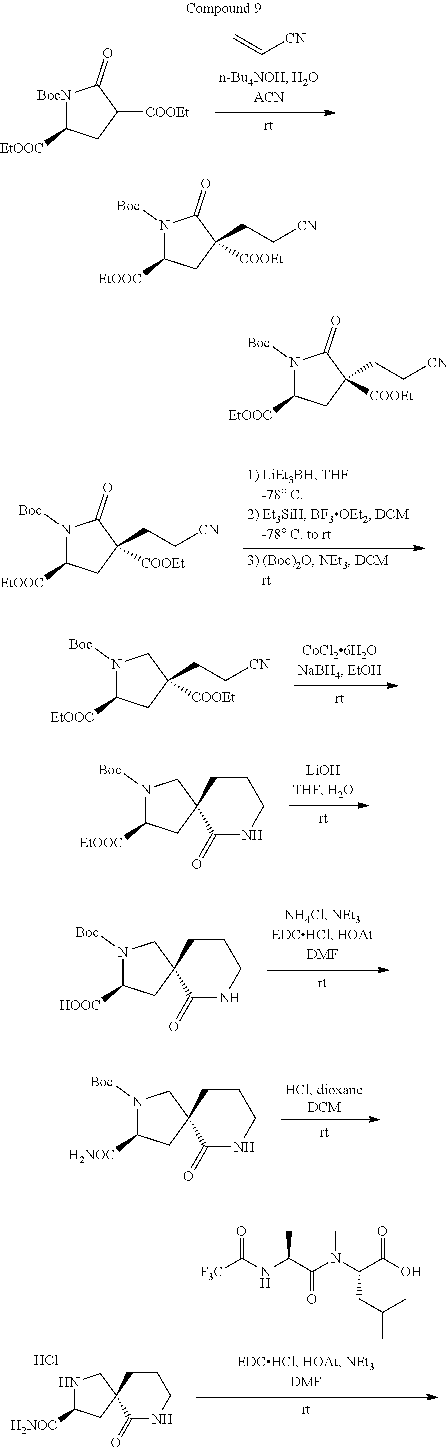

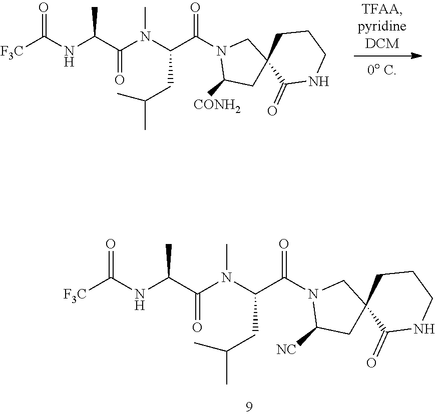 [SMILES: CC(C)C[C@@H](C(=O)N1C[C@@]2(CCCNC2=O)C[C@H]1C(N)=O)N(C)C(=O)[C@H](C)NC(=O)C(F)(F)F ; CC(C)C[C@@H](C(=O)N1C[C@@]2(CCCNC2=O)C[C@H]1C#N)N(C)C(=O)[C@H](C)NC(=O)C(F)(F)F]

To a solution of 1-(t-butyl) 2,4-diethyl (2S)-5-oxopyrrolidine-1,2,4-tricarboxylate (10 g, 30.4 mmol) in ACN (100 mL) cooled to 0° C. were added acrylonitrile (4 mL, 60.8 mmol) and 40% tetra-n-butyl-ammonium hydroxide solution in water (1.5 mL, 2.36 mmol). The mixture was stirred at rt for 2 h. The mixture was diluted with water (50 mL), Et₂O (50 mL) and EA (50 mL). The phases were separated. The organic phase was washed with brine (100 mL), dried over Na₂SO₄. The solids were removed by filtration and the filtrate was concentrated under reduced pressure. The residue was purified by flash chromatography on silica gel using a gradient of EA (12 to 17%) in PE to afford 1-(t-butyl) 2,4-diethyl (2S,4R)-4-(2-cyanoethyl)-5-oxopyrrolidine-1,2,4-tricarboxylate (3.5 g, 30%) as a colorless oil.

The second eluting compound during the purification by flash chromatography was also isolated to afford 1-(t-butyl) 2,4-diethyl (2S,4S)-4-(2-cyanoethyl)-5-oxopyrrolidine-1,2,4-tricarboxylate (5.5 g, 47%) as an off-white solid.

To a solution of 1-(t-butyl) 2,4-diethyl (2S,4R)-4-(2-cyanoethyl)-5-oxopyrrolidine-1,2,4-tricarboxylate (2.0 g, 5.23 mmol) in THF (20 mL) cooled to −78° C. was added dropwise 1M lithium triethyl borohydride solution in THF (6.28 mL, 6.28 mmol). The mixture was stirred at −78° C. for 1 h. The reaction was quenched by addition of sat. NaHCO₃ (20 mL) and hydrogen peroxide (1 mL). The mixture extracted with EA (3×20 mL). The organic phases were combined, washed with brine (10 mL) and dried over Na₂SO₄. The solids were removed by filtration and the filtrate was concentrated under reduced pressure.

The residue (2.0 g) was dissolved in DCM (20 mL) and triethylsilane (0.920 mL, 5.75 mmol) was added. The mixture was stirred at rt for 10 min and was cooled to −78° C. BF₃·OEt₂ (0.710 mL, 0.677 mmol) was added. The mixture was stirred at −78° C. for 30 min. Triethylsilane (0.920 mL, 5.75 mmol) and BF₃·OEt₂ (0.710 mL, 0.677 mmol) were added another time. The mixture was allowed to warm to rt and then stirred overnight at rt, then concentrated under reduced pressure.

The residue (2.0 g) was dissolved in DCM (20 mL) and the mixture was cooled to 0° C. NEt₃ (3.14 mL, 22.4 mmol) and di-t-butyl dicarbonate (1.95 g, 8.96 mmol) were added. The mixture was stirred at rt for 5 h. The mixture was diluted with DCM (20 mL) and washed with water (20 mL). The phases were separated and the organic phase was dried over Na₂SO₄. The solids were removed by filtration and the filtrate was concentrated under reduced pressure. The residue was purified by flash chromatography on silica gel using a gradient of EA (30 to 40%) in PE to afford 1-(t-butyl) 2,4-diethyl (2S,4R)-4-(2-cyanoethyl)pyrrolidine-1,2,4-tricarboxylate (1.2 g, 63% over 3 steps) as a colorless oil.

To a solution of 1-(t-butyl) 2,4-diethyl (2S,4R)-4-(2-cyanoethyl)pyrrolidine-1,2,4-tricarboxylate (1.2 g, 3.26 mmol) in EtOH (15 mL) cooled to 0° C. were added CoCl₂·6H₂O (1.5 g, 6.32 mmol) and NaBH₄ (987 mg, 26.7 mmol). The mixture was stirred at rt for 16 h, then was diluted with 25% NH₄OH (5 mL) and water (10 mL) and extracted with DCM (2×30 mL). The organic phases were combined, dried over Na₂SO₄. The solids were removed by filtration and the filtrate was concentrated under reduced pressure. The residue was purified by flash chromatography on silica gel using a gradient of MeOH (1 to 10%) in DCM to afford 2-(t-butyl) 3-ethyl (3S,5R)-6-oxo-2,7-diazaspiro[4.5]decane-2,3-dicarboxylate (800 mg, 68%) as a colorless oil. LC-MS (ESI, m/z): 327 [M+H]⁺.

To a solution of 2-(t-butyl) 3-ethyl (3S,5R)-6-oxo-2,7-diazaspiro[4.5]decane-2,3-dicarboxylate (800 mg, 2.45 mmol) in THF (5 mL) and water (5 mL) cooled to 0° C. was added LiOH (154 mg, 3.68 mmol). The mixture was stirred at rt for 3 h. The mixture was partially concentrated under reduced pressure to remove the organic solvents. The residue was acidified by addition of 1N HCl until pH ˜4 and extracted with 10% MeOH:DCM (5×10 mL). The organic phases were combined, dried over Na₂SO₄. The solids were removed by filtration and the filtrate was concentrated under reduced pressure to afford (3S,5R)-2-(t-butoxycarbonyl)-6-oxo-2,7-diazaspiro[4.5]decane-3-carboxylic acid (600 mg, 82%) as an off-white solid.

To a solution of (3S,5R)-2-(t-butoxycarbonyl)-6-oxo-2,7-diazaspiro[4.5]decane-3-carboxylic acid (600 mg, 2.01 mmol) in DMF (6 mL) cooled to 0° C. were added EDC-HCl (768 mg, 2.01 mmol), HOAt (273 mg, 2.01 mmol), NEt₃ (0.84 mL, 6.04 mmol) and NH₄Cl (533 mg, 10.1 mmol). The mixture was stirred at rt for 16 h. The mixture was diluted with water (10 mL) and extracted with 10% MeOH:DCM (5×10 mL). The organic phases were combined, dried over Na₂SO₄. The solids were removed by filtration and the filtrate was concentrated under reduced pressure. The residue was purified by flash chromatography on silica gel using a gradient of MeOH (1 to 10%) in DCM to afford t-butyl (3S,5R)-3-carbamoyl-6-oxo-2,7-diazaspiro[4.5]decane-2-carboxylate (500 mg, 83%) as a white solid.

To a solution of t-butyl (3S,5R)-3-carbamoyl-6-oxo-2,7-diazaspiro [4.5]decane-2-carboxylate (300 mg, 1.01 mmol) in DCM (3 mL) cooled to 0° C. was added 4M HCl in dioxane (1.26 mL, 5.04 mmol). The mixture was stirred at rt for 2 h. The mixture was concentrated under reduced pressure and co-evaporated with Et₂O to afford quantitatively (3S,5R)-6-oxo-2,7-diazaspiro[4.5]decane-3-carboxamide hydrochloride as a white solid.

To a solution of N-methyl-N-((2,2,2-trifluoroacetyl)-L-alanyl)-L-leucine (100 mg, 0.320 mmol) in DMF (1 mL) cooled to 0° C. were added (3S,5R)-6-oxo-2,7-diazaspiro[4.5]decane-3-carboxamide hydrochloride (89 mg, 0.384 mmol), EDC·HCl (122 mg, 0.640 mmol), HOAt (43 mg, 0.316 mmol) and NEt₃ (0.134 mL, 0.960 mmol). The mixture was stirred at rt for 16 h. The mixture was diluted with water (5 mL) and extracted with 10% MeOH:DCM (5×10 mL). The organic phases were combined, dried over Na₂SO₄. The solids were removed by filtration and the filtrate was concentrated under reduced pressure. The residue was purified by flash chromatography on C18 using a gradient of ACN (20 to 30%) in 0.1% FA in water to afford (3S,5R)-2-(N-methyl-N-((2,2,2-trifluoroacetyl)-L-alanyl)-L-leucyl)-6-oxo-2,7-diazaspiro[4.5]decane-3-carboxamide (100 mg, 63%) as a white solid.

To a solution of (3S,5R)-2-(N-methyl-N-((2,2,2-trifluoroacetyl)-L-alanyl)-L-leucyl)-6-oxo-2,7-diazaspiro[4.5]decane-3-carboxamide (100 mg, 0.203 mmol) in DCM (1 mL) cooled to 0° C. were added pyridine (0.036 mL, 0.447 mmol) and TFAA (0.031 mL, 0.223 mmol). The mixture was stirred at 0° C. for 1 h. The mixture was diluted with water (5 mL) and extracted with DCM (3×10 mL). The organic phases were combined, washed with sat. NaHCO₃ (10 mL), dried over Na₂SO₄. The solids were removed by filtration and the filtrate was concentrated under reduced pressure. The residue was purified by prep-HPLC (Column: UNISIL-C18 Column, 25×150 mm, 8 m; Mobile Phase A: 0.1% FA in water, Mobile Phase B: ACN; Flow rate: 22 mL/min; Gradient: 15% B to 65% B in 8 min) to afford (S)—N—((S)-1-((3S,5R)-3-cyano-6-oxo-2,7-diazaspiro[4.5]decan-2-yl)-4-methyl-1-oxopentan-2-yl)-N-methyl-2-(2,2,2-trifluoroacetamido)propanamide (50 mg, 52%) as a white solid. ¹H NMR (500 MHz, 363K, DMSO-d₆) δ 9.21 (br. s., 1H), 7.27 (br. s., 1H), 5.23 (s, 1H), 4.75 (m, 2H), 3.75 (m, 1H), 3.42 (m, 1H), 3.09-3.21 (m, 2H), 2.89 (s, 3H), 2.69 (m, 1H), 2.04 (m, 1H), 1.90-1.82 (m, 1H), 1.73-1.83 (m, 2H), 1.64 (m, 2H), 1.49 (m, 2H), 1.28 (d, 3H), 0.79-0.95 (m, 6H). LC-MS (ESI, m/z): 474 [M+H]⁺.

### Example 10

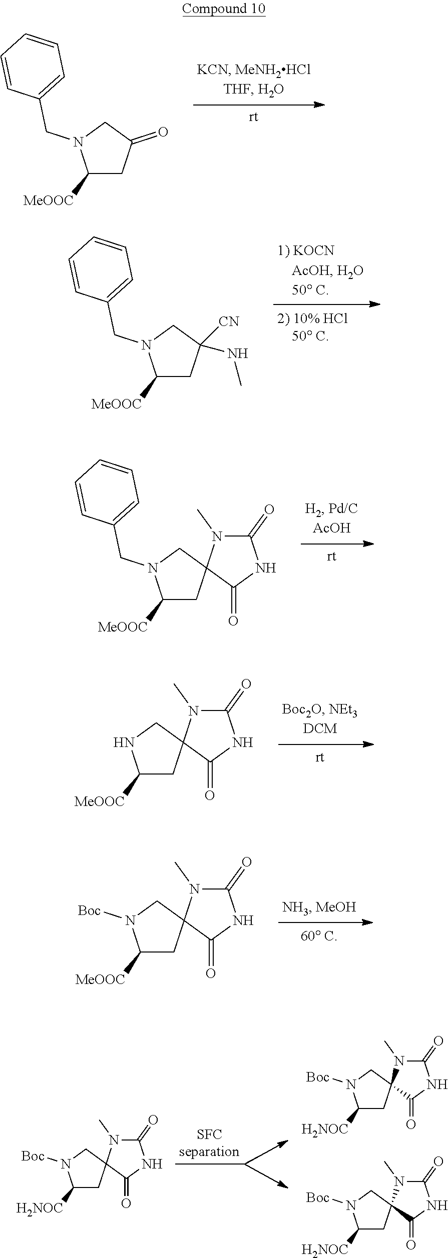

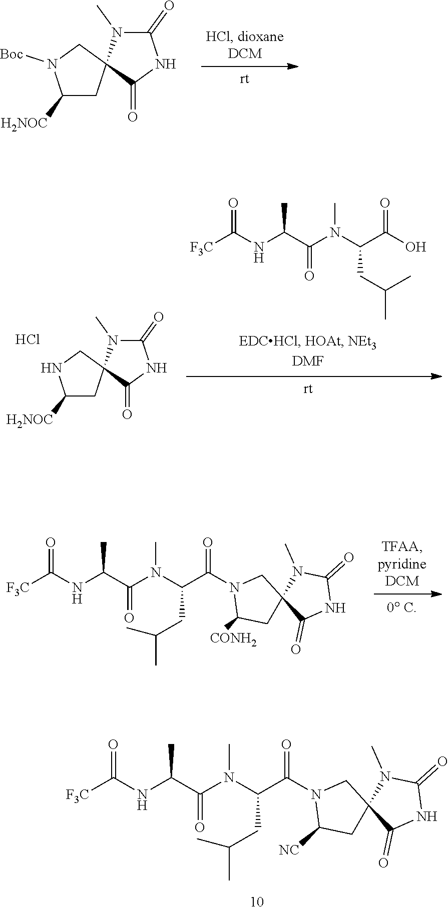 [SMILES: CN1C(=O)NC(=O)[C@]12C[C@@H](C(N)=O)N(C(=O)OC(C)(C)C)C2 ; Cl ; CN1C(=O)NC(=O)[C@@]12CN[C@H](C(N)=O)C2 ; CC(C)C[C@@H](C(=O)O)N(C)C(=O)[C@H](C)NC(=O)C(F)(F)F ; CC(C)C[C@@H](C(=O)N1C[C@]2(C[C@H]1C(N)=O)C(=O)NC(=O)N2C)N(C)C(=O)[C@H](C)NC(=O)C(F)(F)F ; CC(C)C[C@@H](C(=O)N1C[C@]2(C[C@H]1C#N)C(=O)NC(=O)N2C)N(C)C(=O)[C@H](C)NC(=O)C(F)(F)F]

To a solution of methyl (S)-1-benzyl-4-oxopyrrolidine-2-carboxylate (4.2 g, 18.0 mmol) in THF (42 mL) and water (4.2 mL) cooled to 0° C. were added methylamine hydrochloride (1.22 g, 18.0 mmol) and KCN (1.17 g, 18.0 mmol). The mixture was stirred at rt for 2 d. Methylamine hydrochloride (0.243 g, 3.60 mmol) and KCN (0.234 g, 3.59 mmol) were added another time and the mixture was stirred at rt for 12 h. The mixture was diluted with water (50 mL) and extracted with EA (4×50 mL). The organic phases were combined, washed with brine (20 mL), dried over Na₂SO₄. The solids were removed by filtration and the filtrate was concentrated under reduced pressure. The residue was purified by flash chromatography on silica gel using a gradient of EA (20 to 30%) in PE to afford methyl (2S)-1-benzyl-4-cyano-4-(methylamino)pyrrolidine-2-carboxylate (1.9 g, 38%) as a colorless oil.

To a solution of methyl (2S)-1-benzyl-4-cyano-4-(methylamino)pyrrolidine-2-carboxylate (3.3 g, 12.1 mmol) in acetic acid (AcOH) (26 mL) was added dropwise a solution of KOCN (1.96 g, 24.2 mmol) in water (3.3 mL). The mixture was heated at 50° C. for 1 h. After cooling to rt, the mixture was poured into ice/water (100 mL) and extracted with EA (4×50 mL). The organic phases were combined, dried over Na₂SO₄. The solids were removed by filtration and the filtrate was concentrated under reduced pressure. 10% aq. HCl (26 mL) was added to the residue and the mixture was heated at 50° C. for 15 min. The mixture was concentrated under reduced pressure. 20% MeOH in EA (200 mL) was added, and the mixture was filtered through a silica bed. The filtrate was concentrated under reduced pressure to afford methyl (8S)-7-benzyl-1-methyl-2,4-dioxo-1,3,7-triazaspiro[4.4]nonane-8-carboxylate (3.4 g) as an off-white solid.

A mixture of methyl (8S)-7-benzyl-1-methyl-2,4-dioxo-1,3,7-triazaspiro[4.4]nonane-8-carboxylate (5.5 g, 17.6 mmol) and 10% Pd/C (1.1 g) in AcOH (44 mL) was stirred under H₂ atmosphere at rt for 12 h. The mixture was diluted with EtOH (100 mL) and filtered through celite. The solids were washed with EtOH (100 mL) and the filtrate was concentrated under reduced pressure to afford methyl (8S)-1-methyl-2,4-dioxo-1,3,7-triazaspiro[4.4]nonane-8-carboxylate (3.9 g) as a brown oil.

To a suspension of methyl (8S)-1-methyl-2,4-dioxo-1,3,7-triazaspiro[4.4]nonane-8-carboxylate (3.9 g, 17.2 mmol) in DCM (160 mL) cooled to 0° C. were added NEt₃ (7.2 mL, 51.5 mmol) and di-t-butyl dicarbonate (4.12 g, 18.9 mmol). The mixture was stirred at rt for 4 h. The mixture was washed with water (50 mL) and the phases were separated. The aqueous layer was extracted with DCM (2×50 mL). The organic phases were combined, dried over Na₂SO₄. The solids were removed by filtration and the filtrate was concentrated under reduced pressure. The residue was purified by flash chromatography on silica gel using a gradient of EA (40 to 50%) in PE to afford 7-(t-butyl) 8-methyl (8S)-1-methyl-2,4-dioxo-1,3,7-triazaspiro[4.4]nonane-7,8-dicarboxylate (2.2 g, 39% over three steps) as an off-white solid.

A mixture of 7-(t-butyl) 8-methyl (8S)-1-methyl-2,4-dioxo-1,3,7-triazaspiro[4.4]nonane-7,8-dicarboxylate (2.2 g, 6.72 mmol) in 7N NH₃ in MeOH (40 mL) was heated in a sealed tube at 60° C. for 24 h. 7N NH₃ in MeOH (10 mL) was added and the mixture was heated in a sealed tube at 60° C. for 48 h. The mixture was concentrated under reduced pressure. The residue was purified by flash chromatography on silica gel using a gradient of MeOH (5 to 15%) in DCM to afford t-butyl (8S)-8-carbamoyl-1-methyl-2,4-dioxo-1,3,7-triazaspiro[4.4]nonane-7-carboxylate (1.7 g, 81%) as an off-white solid.

t-Butyl (8S)-8-carbamoyl-1-methyl-2,4-dioxo-1,3,7-triazaspiro[4.4]nonane-7-carboxylate (1.5 g) was purified by prep-SFC using the following conditions: Column: Lux Cellulose-4, 3×25 cm, 5 m; Mobile Phase A: CO₂, Mobile Phase B: MeOH; Flow rate: 100 g/min; Elution condition: isocratic 20% B; Column Temperature: 30° C.; Back Pressure: 100 bar. Purification resulted in t-butyl (5S,8S)-8-carbamoyl-1-methyl-2,4-dioxo-1,3,7-triazaspiro[4.4]nonane-7-carboxylate (450 mg) and t-butyl (5R,8S)-8-carbamoyl-1-methyl-2,4-dioxo-1,3,7-triazaspiro[4.4]nonane-7-carboxylate (700 mg).

t-Butyl (5S,8S)-8-carbamoyl-1-methyl-2,4-dioxo-1,3,7-triazaspiro[4.4]nonane-7-carboxylate: SFC: Chiralcel OX-3, 4.6×150 mm, 3 m, 30° C., co-Solvent: 0.5% DEA (diethylamine) in MeOH, hold 8 min at 20%, Rt: 2.39 min. t-Butyl (5R,8S)-8-carbamoyl-1-methyl-2,4-dioxo-1,3,7-triazaspiro[4.4]nonane-7-carboxylate: SFC: Chiralcel OX-3, 4.6×150 mm, 3 m, 30° C., co-Solvent: 0.5% DEA in MeOH, hold 8 min at 20%, Rt: 4.24 min.

To a solution of t-butyl (5R,8S)-8-carbamoyl-1-methyl-2,4-dioxo-1,3,7-triazaspiro[4.4]nonane-7-carboxylate (250 mg, 0.800 mmol) in DCM (5 mL) cooled to 0° C. was added 4M HCl in dioxane (1.0 mL, 4.00 mmol). The mixture was stirred at rt for 3 h. 4M HCl in dioxane (1.0 mL, 4.00 mmol) was added another time and the mixture was stirred at rt for 3 h. The mixture was concentrated under reduced pressure and co-evaporated with Et₂O to afford quantitatively (5R,8S)-1-methyl-2,4-dioxo-1,3,7-triazaspiro[4.4]nonane-8-carboxamide hydrochloride as an off-white solid.

To a solution of N-methyl-N-((2,2,2-trifluoroacetyl)-L-alanyl)-L-leucine (100 mg, 0.320 mmol) in DMF (1 mL) cooled to 0° C. were added (5R,8S)-1-methyl-2,4-dioxo-1,3,7-triazaspiro[4.4]nonane-8-carboxamide hydrochloride (96 mg, 0.384 mmol), EDC·HCl (123 mg, 0.642 mmol), HOAt (44 mg, 0.323 mmol) and NEt₃ (0.130 mL, 0.933 mmol). The mixture was stirred at rt for 18 h. The mixture was diluted with water (5 mL) and extracted with EA (4×10 mL). The organic phases were combined, dried over Na₂SO₄. The solids were removed by filtration and the filtrate was concentrated under reduced pressure. The residue was purified by flash chromatography on C18 using a gradient of ACN in 0.1% FA in water to afford (5R,8S)-1-methyl-7-(N-methyl-N-((2,2,2-trifluoroacetyl)-L-alanyl)-L-leucyl)-2,4-dioxo-1,3,7-triazaspiro[4.4]nonane-8-carboxamide (60 mg, 37%) as a white solid.

To a solution of (5R,8S)-1-methyl-7-(N-methyl-N-((2,2,2-trifluoroacetyl)-L-alanyl)-L-leucyl)-2,4-dioxo-1,3,7-triazaspiro[4.4]nonane-8-carboxamide (60 mg, 0.118 mmol) in DCM (1.2 mL) cooled to 0° C. were added pyridine (0.021 mL, 0.260 mmol) and TFAA (0.018 mL, 0.129 mmol. The mixture was stirred at 0° C. for 1 h. The mixture was diluted with water (3 mL) and extracted with EA (5×5 mL). The organic phases were combined, washed with sat. NaHCO₃ (2 mL), dried over Na₂SO₄. The solids were removed by filtration and the filtrate was concentrated under reduced pressure. The residue was purified by prep-HPLC (Column: Gemini NX C18 Column, 10×250 mm, 5 m; Mobile Phase A: 0.1% FA in water, Mobile Phase B: ACN; Flow rate: 8 mL/min; Gradient: 10% B to 45% B in 9 min) to afford (S)—N—((S)-1-((5R,8S)-8-cyano-1-methyl-2,4-dioxo-1,3,7-triazaspiro[4.4]nonan-7-yl)-4-methyl-1-oxopentan-2-yl)-N-methyl-2-(2,2,2-trifluoroacetamido)propanamide (26 mg, 45%) as a white solid. ¹H NMR (500 MHz, 363K, DMSO-d₆) δ 10.90 (br. s., 1H), 9.32 (br. s., 1H), 5.22 (m, 1H), 5.11 (m, 1H), 4.78 (m, 1H), 4.07 (m, 1H), 3.82 (m, 1H), 3.01 (s, 3H), 2.82 (m, 1H), 2.71 (s, 3H), 2.50-2.55 (m, 1H), 1.64 (m, 2H), 1.51 (m, 1H), 1.30 (d, 3H), 0.92 (d, 3H), 0.87 (d, 3H). LC-MS (ESI, m/z): 489 [M+H]⁺.

### Example 11

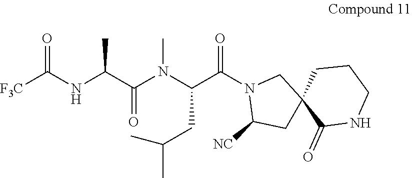 [SMILES: CC(C)C[C@@H](C(=O)N1C[C@]2(CCCNC2=O)C[C@H]1C#N)N(C)C(=O)[C@H](C)NC(=O)C(F)(F)F]

Compound 11 was prepared similarly as described for Compound 9 using 1-(t-butyl) 2,4-diethyl (2S,4S)-4-(2-cyanoethyl)-5-oxopyrrolidine-1,2,4-tricarboxylate in place of 1-(t-butyl) 2,4-diethyl (2S,4R)-4-(2-cyanoethyl)-5-oxopyrrolidine-1,2,4-tricarboxylate. ¹H NMR (500 MHz, 363K, DMSO-d₆) δ 9.24 (br. s., 1H), 7.38 (br. s., 1H), 5.22 (m, 1H), 4.76 (m, 1H), 4.68 (m, 1H), 3.70 (m, 2H), 3.16 (m, 2H), 2.91 (s, 3H), 2.50-2.55 (m, 1H), 2.32 (m, 1H), 1.45-1.75 (m, 7H), 1.20 (d, 3H), 0.90 (d, 3H), 0.87 (d, 3H). LC-MS (ESI, m/z): 474 [M+H]⁺.

### Example 12

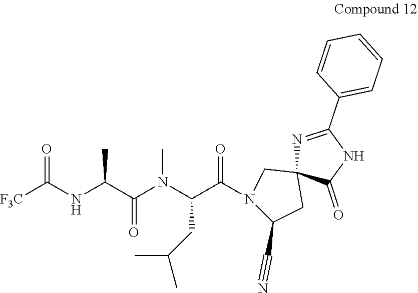 [SMILES: CC(C)C[C@@H](C(=O)N1C[C@@]2(C[C@H]1C#N)N=C(c1ccccc1)NC2=O)N(C)C(=O)[C@H](C)NC(=O)C(F)(F)F]

To a stirred mixture of (5R,8S)-4-oxo-2-phenyl-1,3,7-triazaspiro[4.4]non-1-ene-8-carboxamide (144 mg, 0.558 mmol) and (2S)-2-[(2S)-2-[(t-butoxycarbonyl)amino]-N-methylpropanamido]-4-methylpentanoic acid (176 mg, 0.558 mmol) in ACN (3 mL) were added TCFH (234 mg, 0.836 mmol) and N-methylimidazole (595 mg, 7.24 mmol). The mixture was stirred for 1 h at rt and the reaction was quenched with water (20 mL). The mixture was extracted with EA (3×20 mL). The organic layers were combined, washed with brine (2×20 mL) and dried over anhydrous Na₂SO₄. The solids were removed by filtration and the filtrate was concentrated under reduced pressure to afford the crude product that was purified by C18 column with CH₃CN/water. The desired fraction was concentrated under reduced pressure to provide t-butyl N-[(1S)-1-{[(2S)-1-[(5R,8S)-8-carbamoyl-4-oxo-2-phenyl-1,3,7-triazaspiro[4.4]non-1-en-7-yl]-4-methyl-1-oxopentan-2-yl](methyl)carbamoyl}ethyl]carbamate (128 mg, 39%) as a yellow semi-solid. LC-MS (ESI, m/z): 557 [M+H]⁺.

To a stirred mixture of t-butyl N-[(1S)-1-{[(2S)-1-[(5R,8S)-8-carbamoyl-4-oxo-2-phenyl-1,3,7-triazaspiro[4.4]non-1-en-7-yl]-4-methyl-1-oxopentan-2-yl](methyl)carbamoyl}ethyl]carbamate (120 mg, 0.216 mmol) in 1,4-dioxane (1 mL) was added HCl (2 mL, 4M in dioxane). The mixture was stirred for 1 h at rt and then concentrated under reduced pressure to afford (5R,8S)-7-[(2S)-2-[(2S)-2-amino-N-methylpropanamido]-4-methylpentanoyl]-4-oxo-2-phenyl-1,3,7-triazaspiro[4.4]non-1-ene-8-carboxamide (98 mg, crude) as a yellow solid. LC-MS (ESI, m/z): 457 [M+H]⁺.

To a stirred mixture of (5R,8S)-7-[(2S)-2-[(2S)-2-amino-N-methylpropanamido]-4-methylpentanoyl]-4-oxo-2-phenyl-1,3,7-triazaspiro[4.4]non-1-ene-8-carboxamide (98.0 mg, 0.215 mmol) in MeOH were added triethylamine (130 mg, 1.29 mmol) and ethyl 2,2,2-trifluoroacetate (122 mg, 0.860 mmol). The mixture was stirred for overnight at rt and the reaction was quenched with water (20 mL). The mixture was acidified to pH=3 with HCl (2 M) and then extracted with EA (3×20 mL). The organic layers were combined, washed with brine (2×20 mL) and dried over anhydrous Na₂SO₄. The solids were removed by filtration and the filtrate concentrated under reduced pressure to afford (5R,8S)-7-[(2S)-4-methyl-2-[(2S)—N-methyl-2-(2,2,2-trifluoroacetamido)propanamido]pentanoyl]-4-oxo-2-phenyl-1,3,7-triazaspiro[4.4]non-1-ene-8-carboxamide (116 mg, 88%, crude) as a yellow solid. LC-MS (ESI, m/z): 553 [M+H]⁺.

To a stirred mixture of (5R,8S)-7-[(2S)-4-methyl-2-[(2S)—N-methyl-2-(2,2,2-trifluoroacetamido)propanamido]pentanoyl]-4-oxo-2-phenyl-1,3,7-triazaspiro[4.4]non-1-ene-8-carboxamide (110 mg, 0.199 mmol) in DCM (1.5 mL) were added pyridine (83.6 mg, 1.05 mmol) and trifluoroacetic anhydride (75.2 mg, 0.358 mmol). The mixture was stirred for 2 h at rt and the reaction was quenched with water (15 mL). The mixture was extracted with EA (3×15 mL). The organic layers were combined, washed with brine (2×15 mL) and dried over anhydrous Na₂SO₄. The solids were removed by filtration and the filtrate was concentrated under reduced pressure to afford the crude product that was purified by prep-HPLC (Column: Xselect CSH F-phenyl OBD Column, 19×250 mm, 5 m; Mobile Phase A: water (0.1% FA), Mobile Phase B: ACN; Flow rate: 60 mL/min; Gradient: 37% B to 52% B in 8 min, 52% B; Wave Length: 254; 220 nm; RT1 (min)) to provide (2S)—N-[(2S)-1-[(5R,8S)-8-cyano-4-oxo-2-phenyl-1,3,7-triazaspiro[4.4]non-1-en-7-yl]-4-methyl-1-oxopentan-2-yl]-N-methyl-2-(2,2,2-trifluoroacetamido)propanamide (20.7 mg, 19%) as a white solid. ¹H NMR (400 MHz, 100° C., DMSO-d₆) δ 11.44 (br, 1H), 9.21 (br, 1H), 7.80-8.05 (m, 2H), 7.52-7.70 (m, 1H), 7.49-7.51 (m, 2H), 5.21 (br, 1H), 4.67-4.89 (m, 1H), 4.90-5.10 (m, 1H), 3.40-3.51 (m, 1H), 3.52-4.00 (m, 1H), 2.86-2.94 (m, 3H), 2.54-2.63 (m, 1H), 2.35-2.47 (m, 1H), 1.65-1.81 (m, 1H), 1.40-1.62 (m, 2H), 1.12-1.35 (m, 2H), 1.00-1.11 (m, 1H), 0.69-0.95 (m, 6H). LC-MS (ESI, m/z): 535 [M+H]⁺.

### Example 13

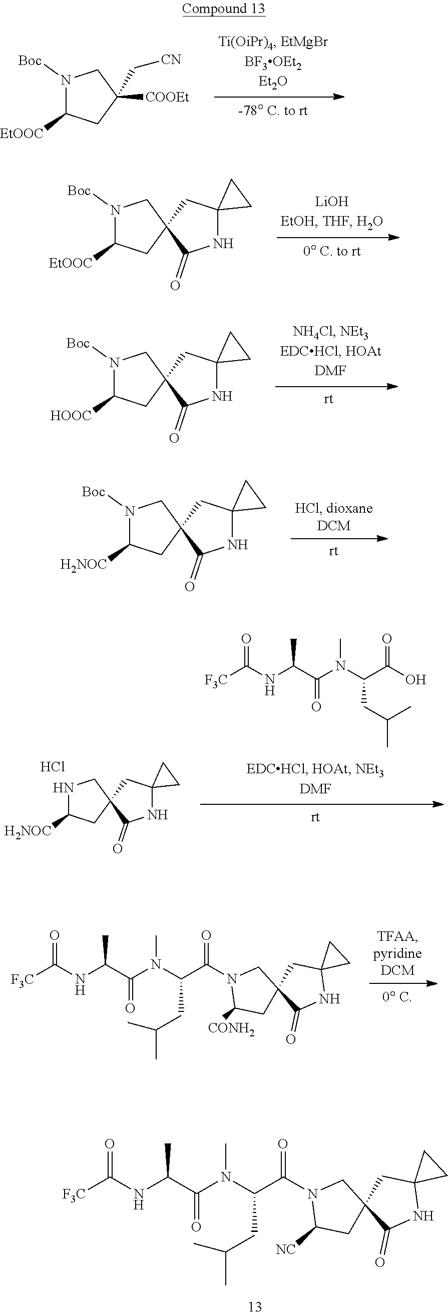

To a solution of 1-(t-butyl) 2,4-diethyl (2S,4R)-4-(cyanomethyl)pyrrolidine-1,2,4-tricarboxylate (200 mg, 0.564 mmol) in Et₂O (4 mL) cooled to −78° C. were added Ti(OiPr)₄ (0.37 mL, 1.24 mmol) and 3M EtMgBr in Et₂O (1.50 mL, 4.50 mmol). The mixture was allowed to warm to rt. After 1 h at rt, BF₃·OEt₂ (0.30 mL, 2.48 mmol) was added. The mixture was stirred at rt for 1 h. The mixture was poured into 28% ammonia solution (5 mL) and then extracted with EA (3×10 mL). The organic phases were combined and dried over Na₂SO₄. The solids were removed by filtration and the filtrate was concentrated under reduced pressure. The residue was purified by flash chromatography on silica gel using a gradient of EA (60 to 70%) in PE and by prep-HPLC (Column: HICHROM 5 C18 Column, 25×150 mm, 5 m; Mobile Phase A: 10 mM NH₄HCO₃ in water, Mobile Phase B: ACN; Flow rate: 22 mL/min; Gradient: 20% B to 65% B in 8 min) to afford 7-(t-butyl) 8-ethyl (5R,8S)-10-oxo-7,11-diazadispiro[2.1.4⁵.2³]undecane-7,8-dicarboxylate (20 mg, 12%) as a yellow oil. LC-MS (ESI, m/z): 339 [M+H]⁺.

To a solution of 7-(t-butyl) 8-ethyl (5R,8S)-10-oxo-7,11-diazadispiro[2.1.4⁵.2³]undecane-7,8-dicarboxylate (300 mg, 0.887 mmol) in EtOH (1 mL), THF (1 mL) and water (1 mL) cooled to 0° C. was added LiOH (56 mg, 1.33 mmol). The mixture was stirred at 0° C. for 1 h and then at rt for 4 h. The mixture was partially concentrated under reduced pressure to remove the organic solvents. The residue was acidified by addition of 1N HCl until pH ˜2 and then extracted with DCM (3×10 mL). The organic phases were combined, dried over Na₂SO₄. The solids were removed by filtration and the filtrate was concentrated under reduced pressure to afford (5R,8S)-7-(t-butoxycarbonyl)-10-oxo-7,11-diazadispiro[2.1.4⁵0.2³]undecane-8-carboxylic acid (250 mg, 90%) as an off-white solid.

To a solution of (5R,8S)-7-(t-butoxycarbonyl)-10-oxo-7,11-diazadispiro[2.1.4⁵.2³]undecane-8-carboxylic acid (150 mg, 0.483 mmol) in DMF (1.5 mL) cooled to 0° C. were added EDC·HCl (183 mg, 0.966 mmol), HOAt (66 mg, 0.483 mmol), NEt₃ (0.20 mL, 1.44 mmol) and NH₄Cl (51 mg, 0.967 mmol). The mixture was stirred at rt for 16 h. The mixture was diluted with water (5 mL) and extracted with 20% MeOH:DCM (3×10 mL). The organic phases were combined, dried over Na₂SO₄. The solids were removed by filtration and the filtrate was concentrated under reduced pressure. The residue was purified by flash chromatography on C18 using a gradient of ACN (15 to 20%) in 0.1% FA in water to afford t-butyl (5R,8S)-8-carbamoyl-10-oxo-7,11-diazadispiro[2.1.4⁵.2³]undecane-7-carboxylate (105 mg, 68%) as an off-white solid.

To a solution of t-butyl (5R,8S)-8-carbamoyl-10-oxo-7,11-diazadispiro[2.1.4⁵.2³]undecane-7-carboxylate (70 mg, 0.226 mmol) in DCM (1 mL) cooled to 0° C. was added 4M HCl in dioxane (0.280 mL, 1.12 mmol). The mixture was stirred at rt for 3 h. The mixture was concentrated under reduced pressure with Et₂O to afford quantitatively (5R,8S)-10-oxo-7,11-diazadispiro[2.1.4⁵.2³]undecane-8-carboxamide hydrochloride as a white solid.

To a solution of N-methyl-N-((2,2,2-trifluoroacetyl)-L-alanyl)-L-leucine (80 mg, 0.256 mmol) in DMF (0.8 mL) cooled to 0° C. were added (5R,8S)-10-oxo-7,11-diazadispiro[2.1.4⁵.2³]undecane-8-carboxamide hydrochloride (55 mg, 0.256 mmol), EDC·HCl (97 mg, 0.512 mmol), HOAt (35 mg, 0.256 mmol) and NEt₃ (0.105 mL, 0.750 mmol). The mixture was stirred at rt for 16 h. The mixture was diluted with water (3 mL) and then extracted with 10% MeOH:DCM (3×10 mL). The organic phases were combined and dried over Na₂SO₄. The solids were removed by filtration and the filtrate was concentrated under reduced pressure. The residue was purified by flash chromatography on C18 using a gradient of ACN (25 to 35%) in 0.1% FA in water to afford (5R,8S)-7-(N-methyl-N-((2,2,2-trifluoroacetyl)-L-alanyl)-L-leucyl)-10-oxo-7,11-diazadispiro[2.1.4⁵.2³]undecane-8-carboxamide (70 mg, 54%) as a white solid.

To a solution of (5R,8S)-7-(N-methyl-N-((2,2,2-trifluoroacetyl)-L-alanyl)-L-leucyl)-10-oxo-7,11-diazadispiro[2.1.4⁵.2³]undecane-8-carboxamide (70 mg, 0.139 mmol) in DCM (0.7 mL) cooled to 0° C. were added pyridine (0.025 mL, 0.305 mmol) and TFAA (0.012 mL, 0.153 mmol). The mixture was stirred at 0° C. for 1 h. The mixture was diluted with water (3 mL) and then extracted with DCM (3×5 mL). The organic phases were combined, washed with brine (2×5 mL) and dried over Na₂SO₄. The solids were removed by filtration and the filtrate was concentrated under reduced pressure. The residue was purified by prep-HPLC (Column: X-BRIDGE-C18 Column, 25×150 mm, 5 m; Mobile Phase A: 10 mM NH₄HCO₃ in water, Mobile Phase B: ACN; Flow rate: 22 mL/min; Gradient: 20% B to 60% B in 9 min) to afford (S)—N—((S)-1-((5R,8S)-8-cyano-10-oxo-7,11-diazadispiro[2.1.4⁵.2³]undecan-7-yl)-4-methyl-1-oxopentan-2-yl)-N-methyl-2-(2,2,2-trifluoroacetamido)propanamide (35 mg, 52%) As a white solid. ¹H NMR (500 MHz, 364K, DMSO-d₆) δ 9.30 (br. s., 1H), 7.76 (s, 1H), 5.17 (m, 1H), 4.75 (m, 2H), 3.83 (m, 1H), 3.62 (m, 1H), 2.96 (s, 3H), 2.31-2.45 (m, 2H), 1.91-2.06 (m, 2H), 1.61 (m, 2H), 1.50 (m, 1H), 1.27 (d, 3H), 0.91 (d, 3H), 0.87 (d, 3H), 0.70-0.78 (m, 2H), 0.58-0.67 (m, 2H). LC-MS (ESI, m/z): 486 [M+H]⁺.

### Example 14

### Compounds 14a and 14b

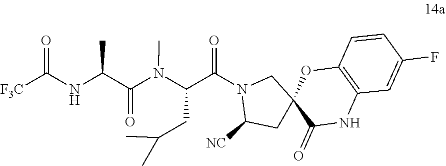 [SMILES: CC(C)C[C@@H](C(=O)N1C[C@@]2(C[C@H]1C#N)Oc1ccc(F)cc1NC2=O)N(C)C(=O)[C@H](C)NC(=O)C(F)(F)F]

To a stirred mixture of t-butyl (2R,5'S)-5′-carbamoyl-6-fluoro-3-oxo-3,4-dihydrospiro[benzo[b][1,4]oxazine-2,3′-pyrrolidine]-1′-carboxylate (200 mg, 0.547 mmol) in 1,4-dioxane (1 mL) was added hydrogen chloride (5 mL, 4M in 1,4-dioxane). The mixture was stirred for 2 h at rt and concentrated under reduced pressure to obtain (2R,5'S)-6-fluoro-3-oxo-3,4-dihydrospiro[benzo[b][1,4]oxazine-2,3′-pyrrolidine]-5′-carboxamide (145 mg, crude) as a yellow solid. LC-MS (ESI, m/z): 266 [M+H]⁺.

To a stirred mixture of (2R,5'S)-6-fluoro-3-oxo-3,4-dihydrospiro[benzo[b][1,4]oxazine-2,3′-pyrrolidine]-5′-carboxamide (145 mg, 0.547 mmol), N-((benzyloxy)carbonyl)-N-methyl-L-leucine (157 mg, 0.563 mmol), HATU (214 mg, 0.563 mmol) in DCM (4 mL) and DMF (1 mL) was added N-methylmorpholine (165 mg, 1.64 mmol). The mixture was stirred for 2 h at rt and the reaction was quenched with water (50 mL). The mixture was extracted with EA (3×50 mL). The organic layers were combined, washed with brine (2×50 mL) and dried over anhydrous sodium sulfate. The solids were removed by filtration and the filtrate was concentrated under reduced pressure to afford the crude product. The crude product was purified by TLC (EA=100) to afford of benzyl ((S)-1-((2R,5'S)-5′-carbamoyl-6-fluoro-3-oxo-3,4-dihydrospiro[benzo[b][1,4]oxazine-2,3′-pyrrolidin]-1′-yl)-4-methyl-1-oxopentan-2-yl)(methyl)carbamate (150 mg, 52%) as a yellow solid. ¹H NMR (400 MHz, DMSO-d₆) δ 10.99-11.23 (m, 1H), 7.13-7.57 (m, 6H), 6.58-7.07 (m, 4H), 4.56-5.04 (m, 3H), 4.27-4.48 (m, 1H), 3.99-4.07 (m, 1H), 3.69-3.93 (m, 1H), 2.72-2.85 (s, 3H), 2.42-2.48 (m, 1H), 2.20-2.37 (m, 1H), 1.28-1.67 (m, 3H), 0.73-0.92 (m, 6H). LC-MS (ESI, m/z): 527 [M+H]⁺.

To a stirred mixture of benzyl ((S)-1-((2R,5'S)-5′-carbamoyl-6-fluoro-3-oxo-3,4-dihydrospiro[benzol[b][1,4]oxazine-2,3′-pyrrolidin]-1′-yl)-4-methyl-1-oxopentan-2-yl)(methyl)carbamate (140 mg, 0.266 mmol) in CH₃OH (3 mL) was added 10% palladium on activated carbon (70 mg). The mixture was stirred for 1 h at rt under hydrogen. The mixture was filtered through a celite pad and washed with DCM (3×30 mL). The filtrate was concentrated under reduced pressure to afford (2R,5'S)-6-fluoro-1′-(methyl-L-leucyl)-3-oxo-3,4-dihydrospiro[benzo[b][1,4]oxazine-2,3′-pyrrolidine]-5′-carboxamide (100 mg, crude) as a white solid. LC-MS (ESI, m/z): 393 [M+H]⁺.

To a stirred mixture of (2R,5'S)-6-fluoro-1′-(methyl-L-leucyl)-3-oxo-3,4-dihydrospiro[benzo[b][1,4]oxazine-2,3′-pyrrolidine]-5′-carboxamide (100 mg, 0.255 mmol), ((benzyloxy)carbonyl)-L-alanine (56.9 mg, 0.255 mmol) and HATU (101 mg, 0.265 mmol) in DCM (0.25 mL) and dimethylformamide (1 mL) was added N-methylmorpholine (51.6 mg, 0.510 mmol). The mixture was stirred for 2 h at rt and the reaction was quenched with water (50 mL). The mixture was extracted with EA (3×50 mL). The organic layers were combined, washed with brine (2×20 mL) and dried over anhydrous sodium sulfate. The solids were removed by filtration and the filtrate was concentrated under reduced pressure to afford the crude product. The crude product was purified by TLC (CH₃OH:DCM=1:15) to afford of benzyl ((S)-1-(((S)-1-((2R,5'S)-5′-carbamoyl-6-fluoro-3-oxo-3,4-dihydrospiro[benzo[b][1,4]oxazine-2,3′-pyrrolidin]-1′-yl)-4-methyl-1-oxopentan-2-yl)(methyl)amino)-1-oxopropan-2-yl)carbamate (60.0 mg, 39%) as a yellow solid. ¹H NMR (400 MHz, DMSO-d₆) δ 10.97-11.22 (m, 1H), 7.44-7.57 (m, 2H), 7.25-7.37 (m, 5H), 6.94-7.05 (m, 2H), 6.75-6.83 (m, 1H), 6.67-6.74 (m, 1H), 5.10-5.20 (m, 1H), 4.95-5.06 (m, 2H), 4.26-4.43 (m, 2H), 3.81-3.95 (m, 2H), 2.80-2.94 (s, 3H), 2.39-2.48 (m, 1H), 2.09-2.25 (m, 1H), 1.35-1.54 (m, 3H), 0.75-0.96 (m, 9H). LC-MS (ESI, m/z): 598 [M+H]⁺.

To a stirred mixture of benzyl ((S)-1-(((S)-1-((2R,5'S)-5′-carbamoyl-6-fluoro-3-oxo-3,4-dihydrospiro[benzo[b][1,4]oxazine-2,3′-pyrrolidin]-1′-yl)-4-methyl-1-oxopentan-2-yl)(methyl)amino)-1-oxopropan-2-yl)carbamate (60.0 mg, 0.100 mmol) in CH₃OH (2 mL) was added 10% palladium on activated carbon (30 mg). The mixture was stirred for 1 h at rt under hydrogen. The mixture was filtered through a celite pad and washed with DCM (3×30 mL). The filtrate was concentrated under reduced pressure to afford (2R,5'S)-1′-(N-(L-alanyl)-N-methyl-L-leucyl)-6-fluoro-3-oxo-3,4-dihydrospiro[benzo[b][1,4]oxazine-2,3′-pyrrolidine]-5′-carboxamide (50 mg, crude) as a white solid. LC-MS (ESI, m/z): 464 [M+H]⁺.

To a mixture of (2R,5'S)-1′-(N-(L-alanyl)-N-methyl-L-leucyl)-6-fluoro-3-oxo-3,4-dihydrospiro[benzo[b][1,4]oxazine-2,3′-pyrrolidine]-5′-carboxamide (50 mg, 0.108 mmol) in CH₃OH (1.5 mL) were added ethyl 2,2,2-trifluoroacetate (153 mg, 1.08 mmol) and trimethylamine (131 mg, 1.30 mmol). The mixture was stirred overnight at rt and then acidified to pH=3 with HCl (aq., 1M). The aqueous layer was extracted with EA (3×30 mL). The organic layers were combined, washed with brine (2×10 mL) and dried over anhydrous sodium sulfate. The mixture was concentrated under reduced pressure to afford (2R,5'S)-6-fluoro-1′-(N-methyl-N-((2,2,2-trifluoroacetyl)-L-alanyl)-L-leucyl)-3-oxo-3,4-dihydrospiro[benzo[b][1,4]oxazine-2,3′-pyrrolidine]-5′-carboxamide (45 mg, crude) as a yellow solid. LC-MS (ESI, m/z): 560 [M+H]⁺.

To a mixture of (2R,5'S)-6-fluoro-1′-(N-methyl-N-((2,2,2-trifluoroacetyl)-L-alanyl)-L-leucyl)-3-oxo-3,4-dihydrospiro[benzo[b][1,4]oxazine-2,3′-pyrrolidine]-5′-carboxamide (50.0 mg, 0.089 mmol) in DCM (1 mL) were added pyridine (35.3 mg, 0.445 mmol) and trifluoroacetic anhydride (33.8 mg, 0.160 mmol). The mixture was stirred for 2 h at rt and the reaction was quenched with water (10 mL). The mixture was extracted with DCM (3×20 mL). The organic layers were combined, washed with brine (2×10 mL) and dried over anhydrous sodium sulfate. The solids were removed by filtration and the filtrate was concentrated under reduced pressure to afford the crude product. The crude product was purified by prep-HPLC (Column: Xselect CSH Prep OBD C18 Column, 30×150 mm, 5 m; Mobile Phase A: Water (0.1% FA), Mobile Phase B: ACN; Flow rate: 60 mL/min; Gradient: 46% B to 56% B in 8 min; Wave Length: 220 nm) to provide (S)—N—((S)-1-((2R,5'S)-5′-cyano-6-fluoro-3-oxo-3,4-dihydrospiro[benzo[b][1,4]oxazine-2,3′-pyrrolidin]-1′-yl)-4-methyl-1-oxopentan-2-yl)-N-methyl-2-(2,2,2-trifluoroacetamido)propanamide (6.9 mg, 14%) as a white solid. ¹H NMR (400 MHz, 80° C., DMSO-d₆) δ 10.86 (br, 1H), 9.31 (br, 1H), 6.86 (br, 1H), 6.55-6.80 (m, 2H), 4.80-5.30 (m, 2H), 4.50-4.70 (m, 1H), 3.70-4.15 (m, 2H), 3.80-3.95 (m, 3H), 2.50-3.80 (m, 2H), 1.30-1.70 (m, 3H), 0.90-1.30 (m, 3H), 0.60-0.90 (m, 6H). LC-MS (ESI, m/z): 564 [M+Na]⁺.

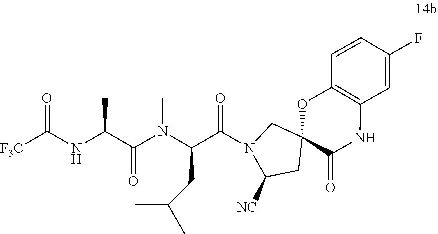 [SMILES: CC(C)C[C@H](C(=O)N1C[C@@]2(C[C@H]1C#N)Oc1ccc(F)cc1NC2=O)N(C)C(=O)[C@H](C)NC(=O)C(F)(F)F]

To a mixture of 1-(t-butyl) 2-methyl (2S,4S)-4-hydroxy-4-(trichloromethyl)pyrrolidine-1,2-dicarboxylate (3.00 g, 8.27 mmol) and 2-bromo-4-fluorophenol (3.16 g, 16.5 mmol) in acetone (30 mL) at 0° C. was added and NaOH (2.00 g, 49.6 mmol). The mixture was stirred overnight at rt and the reaction was quenched with water (200 mL). The mixture was extracted with EA (3×50 mL). The aqueous layer was adjusted to pH=5 with HCl (2 M) and then extracted with EA (3×250 mL). The organic layers were combined, washed with brine (2×200 mL) and dried over anhydrous sodium sulfate. The solids were removed by filtration and the filtrate was concentrated under reduced pressure to provide (2S,4R)-4-(2-bromo-4-fluorophenoxy)-1-(t-butoxycarbonyl)pyrrolidine-2,4-dicarboxylic acid (3.0 g, crude) as a yellow oil. LC-MS (ESI, m/z): 348 [M−Boc+H]⁺.

To a mixture of (2S,4R)-4-(2-bromo-4-fluorophenoxy)-1-(t-butoxycarbonyl)pyrrolidine-2,4-dicarboxylic acid (3.0 g, 6.71 mmol), 1-hydroxybenzotrizole (5.44 g, 40.3 mmol) and 3-(((ethylimino)methylene)amino)-N,N-dimethylpropan-1-amine (6.44 g, 33.6 mmol) in THF (30 mL) was added NH₃·H₂O (50 mL) at 0° C. The mixture was stirred for 3 h at rt and the reaction was quenched with water (60 mL). The mixture was extracted with EA (3×300 mL). The organic layers were combined, washed with brine (2×150 mL) and dried over anhydrous sodium sulfate. The solids were removed by filtration and the filtrate was concentrated under reduced pressure to afford the crude product. The crude product was chromatographed on a silica gel column with CH₃OH:DCM (9:91) to provide t-butyl (2S,4R)-4-(2-bromo-4-fluorophenoxy)-2,4-dicarbamoylpyrrolidine-1-carboxylate (700 mg, 23%) as a reddish brown solid. ¹H NMR (400 MHz, DMSO-d₆) δ 7.94-8.08 (m, 1H), 7.57-7.77 (m, 2H), 7.21-7.53 (m, 2H), 6.78-7.10 (m, 2H), 4.08-4.27 (m, 1H), 3.84-3.97 (m, 1H), 3.60-3.73 (m, 1H), 2.52-2.63 (m, 1H), 2.12-2.41 (m, 1H), 1.21-1.36 (m, 9H). LC-MS (ESI, m/z): 446 [M+H]⁺.

To a stirred mixture of t-butyl (2S,4R)-4-(2-bromo-4-fluorophenoxy)-2,4-dicarbamoylpyrrolidine-1-carboxylate (700 mg, 1.57 mmol), copper(I) iodide (179 mg, 0.942 mmol) and cesium carbonate (1.02 g, 3.14 mmol) in THF (7 mL) was added N,N′-dimethyl-1,2-ethanediamine (263 mg, 2.98 mmol) at rt under nitrogen. The mixture was stirred for 3 h at 70° C. and then filtered. The filter cake was washed with DCM (3×20 mL) and the filtrate was concentrated under reduced pressure to afford the crude product. The crude product was chromatographed on a silica gel column with CH₃OH:DCM (7:93) to provide t-butyl (2R,5'S)-5′-carbamoyl-6-fluoro-3-oxo-3,4-dihydrospiro[benzo[b][1,4]oxazine-2,3′-pyrrolidine]-1′-carboxylate (220 mg, 38%) as a grey solid. ¹H NMR (400 MHz, DMSO-d₆) δ 10.86-11.16 (m, 1H), 7.37-7.61 (m, 1H), 6.92-7.18 (m, 2H), 6.60-6.90 (m, 2H), 4.10-4.36 (m, 1H), 3.52-3.83 (m, 2H), 2.41-2.47 (m, 1H), 2.19-2.40 (m, 1H), 1.28-1.47 (m, 9H). LC-MS (ESI, m/z): 310 [M−56+H]⁺.

To a stirred mixture of t-butyl (2R,5'S)-5′-carbamoyl-6-fluoro-3-oxo-3,4-dihydrospiro[benzo[b][1,4]oxazine-2,3′-pyrrolidine]-1′-carboxylate (140 mg, 0.383 mmol) in DCM (3 mL) was added trifluoroacetic acid (TFA) (1 mL) at rt. The mixture was stirred for 2 h at rt and concentrated under reduced pressure to afford (2R,5'S)-6-fluoro-3-oxo-3,4-dihydrospiro[benzo[b][1,4]oxazine-2,3′-pyrrolidine]-5′-carboxamide (101 mg, crude) as a yellow oil. LC-MS (ESI, m/z): 266 [M+H]⁺.

To a mixture of (2R,5'S)-6-fluoro-3-oxo-3,4-dihydrospiro[benzo[b][1,4]oxazine-2,3′-pyrrolidine]-5′-carboxamide (101 mg, 0.380 mmol) and N-((t-butoxycarbonyl)-L-alanyl)-N-methyl-L-leucine (120 mg, 0.380 mmol) in CH₃CN (2 mL) were added TCFH (139 mg, 0.494 mmol) and then N-methylimidazole (312 mg, 3.80 mmol) at 0° C. The mixture was stirred for 2 h at rt. The mixture was purified by C18 column with CH₃CN:Water (0.05% TFA). The fraction was concentrated under reduced pressure to provide t-butyl ((2S)-1-((1-((2R,5'S)-5′-carbamoyl-6-fluoro-3-oxo-3,4-dihydrospiro[benzo[b][1,4]oxazine-2,3′-pyrrolidin]-1′-yl)-4-methyl-1-oxopentan-2-yl)(methyl)amino)-1-oxopropan-2-yl)carbamate (110 mg, 51%) as a yellow solid. ¹H NMR (400 MHz, DMSO-d₆) δ 10.77-11.25 (m, 1H), 7.25-7.61 (m, 1H), 6.59-7.20 (m, 5H), 5.01-5.25 (m, 1H), 4.25-4.55 (m, 3H), 3.45-3.69 (m, 1H), 2.67-3.06 (m, 3H), 1.93-2.48 (m, 2H), 0.75-1.66 (m, 21H). LC-MS (ESI, m/z): 564 [M+H]⁺.

To a stirred mixture of t-butyl ((2S)-1-((1-((2R,5'S)-5′-carbamoyl-6-fluoro-3-oxo-3,4-dihydrospiro[benzo[b][1,4]oxazine-2,3′-pyrrolidin]-1′-yl)-4-methyl-1-oxopentan-2-yl)(methyl)amino)-1-oxopropan-2-yl)carbamate (110 mg, 0.195 mmol) in DCM (3 mL) was added TFA (1 mL) at rt. The mixture was stirred for 2 h at rt and then concentrated under reduced pressure to afford (2R,5'S)-1′-(N-(L-alanyl)-N-methylleucyl)-6-fluoro-3-oxo-3,4-dihydrospiro[benzo[b][1,4]oxazine-2,3′-pyrrolidine]-5′-carboxamide (90 mg, crude) as a yellow oil. LC-MS (ESI, m/z): 464 [M+H]⁺.

To a mixture of (2R,5'S)-1′-(N-(L-alanyl)-N-methylleucyl)-6-fluoro-3-oxo-3,4-dihydrospiro[benzo[b][1,4]oxazine-2,3′-pyrrolidine]-5′-carboxamide (90.0 mg, 0.194 mmol) in CH₃OH (2 mL) were added ethyl 2,2,2-trifluoroacetate (276 mg, 1.94 mmol) and trimethylamine (236 mg, 2.33 mmol). The mixture was stirred overnight at rt and then acidified to pH=3 with HCl (1M). The aqueous layer was extracted with EA (3×30 mL). The organic layers were combined, washed with brine (2×10 mL) and dried over anhydrous sodium sulfate. The mixture was concentrated under reduced pressure to afford (2R,5'S)-6-fluoro-1′-(N-methyl-N-((2,2,2-trifluoroacetyl)-L-alanyl)leucyl)-3-oxo-3,4-dihydrospiro[benzo[b][1,4]oxazine-2,3′-pyrrolidine]-5′-carboxamide (100 mg, crude) as a yellow solid. LC-MS (ESI, m/z): 560 [M+H]⁺.

To a mixture of (2R,5'S)-6-fluoro-1′-(N-methyl-N-((2,2,2-trifluoroacetyl)-L-alanyl)leucyl)-3-oxo-3,4-dihydrospiro[benzo[b][1,4]oxazine-2,3′-pyrrolidine]-5′-carboxamide (100 mg, 0.179 mmol) in DCM (2 mL) were added pyridine (56.6 mg, 0.716 mmol) and trifluoroacetic anhydride (67.6 mg, 0.322 mmol). The mixture was stirred for 2 h at rt and the reaction was quenched with water (10 mL). The mixture was extracted with DCM (3×20 mL). The organic layers were combined, washed with brine (2×10 mL) and dried over anhydrous sodium sulfate. The solids were removed by filtration and the filtrate was concentrated under reduced pressure to afford the crude product. The crude product was purified by prep-HPLC (Column: Xselect CSH Prep C18 OBD Column, 19×250 mm, 5 m; Mobile Phase A: Water (0.1% FA), Mobile Phase B: ACN; Flow rate: 25 mL/min; Gradient: 38% B to 68% B in 10 min; Wave Length: 220 nm) to provide (S)—N—((R)-1-((2R,5'S)-5′-cyano-6-fluoro-3-oxo-3,4-dihydrospiro[benzo[b][1,4]oxazine-2,3′-pyrrolidin]-1′-yl)-4-methyl-1-oxopentan-2-yl)-N-methyl-2-(2,2,2-trifluoroacetamido)propanamide (14.1 mg, 14%, major isomer) as a white solid. ¹H NMR (400 MHz, 80° C., DMSO-d₆) δ 10.30-11.40 (br, 1H), 9.00-10.00 (br, 1H), 6.60-7.10 (m, 3H), 4.50-5.60 (m, 3H), 3.40-4.30 (m, 2H), 2.85-3.00 (m, 3H), 2.65-2.85 (m, 2H), 1.60-1.80 (m, 1H), 1.35-1.59 (m, 2H), 1.00-1.34 (m, 3H), 0.60-0.99 (m, 6H). LC-MS (ESI, m/z): 564 [M+Na+H]⁺.

### Example 15

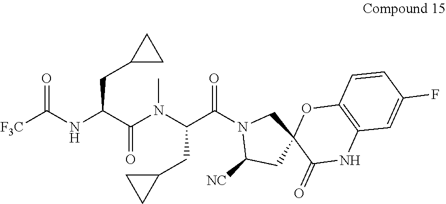 [SMILES: CN(C(=O)[C@H](CC1CC1)NC(=O)C(F)(F)F)[C@@H](CC1CC1)C(=O)N1C[C@@]2(C[C@H]1C#N)Oc1ccc(F)cc1NC2=O]

To a mixture of (2R,5'S)-6-fluoro-3-oxo-3,4-dihydrospiro[benzo[b][1,4]oxazine-2,3′-pyrrolidine]-5′-carboxamide hydrochloride (200 mg, 0.664 mmol), (S)-2-((t-butoxycarbonyl)(methyl)amino)-3-cyclopropylpropanoic acid (162 mg, 0.664 mmol) and HATU (278 mg, 0.730 mmol) in DMF (1.8 mL) and DCM (0.3 mL) was added 4-methylmorpholine (336 mg, 3.32 mmol) at 0° C. The mixture was stirred for 1 h at rt and the reaction was quenched with water (10 mL). The mixture was concentrated under reduced pressure to remove DCM and purified by C18 column with CH₃CN:Water (0.05% TFA), (52%). The fraction was concentrated under reduced pressure to provide t-butyl ((S)-1-((2R,5'S)-5′-carbamoyl-6-fluoro-3-oxo-3,4-dihydrospiro[benzo[b][1,4]oxazine-2,3′-pyrrolidin]-1′-yl)-3-cyclopropyl-1-oxopropan-2-yl)(methyl)carbamate (180 mg, 59%) as a yellow solid. LCMS (ESI, m/z): 491 [M+H]⁺.

To a mixture of t-butyl ((S)-1-((2R,5'S)-5′-carbamoyl-6-fluoro-3-oxo-3,4-dihydrospiro[benzo[b][1,4]oxazine-2,3′-pyrrolidin]-1′-yl)-3-cyclopropyl-1-oxopropan-2-yl)(methyl)carbamate (180 mg, 0.367 mmol) in 1,4-dioxane (2 mL) was added hydrogen chloride (3 mL, 4 M in 1,4-dioxane) at rt. The mixture was stirred for 1 h and then concentrated under reduced pressure to afford (2R,5'S)-1′-((S)-3-cyclopropyl-2-(methylamino)propanoyl)-6-fluoro-3-oxo-3,4-dihydrospiro[benzo[b][1,4]oxazine-2,3′-pyrrolidine]-5′-carboxamide hydrochloride (180 mg, crude) as a light yellow solid. LCMS (ESI, m/z): 391 [M+H]⁺.

To a solution of (2R,5'S)-1′-((S)-3-cyclopropyl-2-(methylamino)propanoyl)-6-fluoro-3-oxo-3,4-dihydrospiro[benzo[b][1,4]oxazine-2,3′-pyrrolidine]-5′-carboxamide HCl (180 mg, 0.461 mmol), (2S)-2-[(t-butoxycarbonyl)amino]-3-cyclopropylpropanoic acid (116 mg, 0.507 mmol), HATU (192 mg, 0.507 mmol) in DCM (1.2 mL) and DMF (0.2 mL) was added 4-methylmorpholine (256 mg, 2.54 mmol) at 0° C. The mixture was stirred for 1 h at rt and the reaction was quenched with water (10 mL). The mixture was concentrated under reduced pressure and purified by C18 column with CH₃CN:Water (0.05% TFA), (58%). The fraction was concentrated under reduced pressure to provide t-butyl ((S)-1-(((S)-1-((2R,5'S)-5′-carbamoyl-6-fluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-3-cyclopropyl-1-oxopropan-2-yl)carbamate (130 mg, 47%).

To a mixture of t-butyl ((S)-1-(((S)-1-((2R,5'S)-5′-carbamoyl-6-fluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-3-cyclopropyl-1-oxopropan-2-yl)carbamate (130 mg, 0.216 mmol) in 1,4-dioxane (1 mL) was added hydrogen chloride (3 mL, 4 M in 1,4-dioxane) stirred at rt. The mixture was stirred for 1 h and then concentrated under reduced pressure to afford (2R,5'S)-1′-((S)-2-((S)-2-amino-3-cyclopropyl-N-methylpropanamido)-3-cyclopropylpropanoyl)-6-fluoro-3-oxo-3,4-dihydrospiro[benzo[b][1,4]oxazine-2,3′-pyrrolidine]-5′-carboxamide HCl (130 mg, crude) as a yellow solid. LCMS (ESI, m/z): 502 [M+H]⁺.

To a mixture of (2R,5'S)-1′-((S)-2-((S)-2-amino-3-cyclopropyl-N-methylpropanamido)-3-cyclopropylpropanoyl)-6-fluoro-3-oxo-3,4-dihydrospiro[benzo[b][1,4]oxazine-2,3′-pyrrolidine]-5′-carboxamide HCl (130 mg, 0.259 mmol) in methanol (1 mL) were added triethylamine (314 mg, 3.11 mmol) and ethyl 2,2,2-trifluoroacetate (368 mg, 2.59 mmol) at rt. The mixture was stirred overnight and the reaction was quenched with water (10 mL). The mixture was concentrated under reduced pressure to remove CH₃OH and then acidified to pH=5 with HCl (1 M). The mixture was extracted with EA (3×50 mL). The organic layers were combined, washed with brine (2×20 mL) and dried over anhydrous sodium sulfate. The solids were removed by filtration and the filtrate was concentrated under reduced pressure to afford (2R,5'S)-1′-((S)-3-cyclopropyl-2-((S)-3-cyclopropyl-N-methyl-2-(2,2,2-trifluoroacetamido)propanamido)propanoyl)-6-fluoro-3-oxo-3,4-dihydrospiro[benzo[b][1,4]oxazine-2,3′-pyrrolidine]-5′-carboxamide (60 mg, crude) as a yellow solid. LCMS (ESI, m/z): 333 (fragment peak).

To a solution of (2R,5'S)-1′-((S)-3-cyclopropyl-2-((S)-3-cyclopropyl-N-methyl-2-(2,2,2-trifluoroacetamido)propanamido)propanoyl)-6-fluoro-3-oxo-3,4-dihydrospiro[benzo[b][1,4]oxazine-2,3′-pyrrolidine]-5′-carboxamide (60 mg, 0.100 mmol) in DCM (1 mL) were added pyridine (39.5 mg, 0.500 mmol) and trifluoroacetyl 2,2,2-trifluoroacetate (37.8 mg, 0.180 mmol). The mixture was stirred 1 h at rt and the reaction was quenched with water (10 mL). The mixture was extracted with DCM (3×30 mL). The organic layers were combined, washed with brine (2×10 mL) and dried over anhydrous sodium sulfate. The solids were removed by filtration and the filtrate was concentrated under reduced pressure. The crude product was purified by prep-HPLC (Column: Xselect CSH Prep Fluoro-Phenyl Column 19×250 mm 5 m; Mobile Phase A: Water (0.1% FA), Mobile Phase B: ACN; Flow rate: 25 mL/min; Gradient: 35% B to 65% B in 10 min; Wave Length: 220 nm) to provide (S)—N—((S)-1-((2R,5'S)-5′-cyano-6-fluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-N-methyl-2-(2,2,2-trifluoroacetamido)propanamide (14.9 mg, 25%) as a white solid. ¹H NMR (400 MHz, 70° C., DMSO-d₆) δ 10.90 (br, 1H), 9.39 (br, 1H), 6.75-7.00 (m, 1H), 6.55-6.75 (m, 2H), 5.00-5.35 (m, 1H), 4.75-5.00 (m, 1H), 4.50-4.75 (m, 1H), 3.99 (br, 2H), 2.93 (s, 3H), 2.60-2.75 (m, 1H), 2.45-2.55 (m, 1H), 1.50-1.75 (m, 2H), 1.22-1.50 (m, 2H), 0.45-0.70 (m, 2H), 0.12-0.45 (m, 4H), −0.10-0.12 (m, 2H), −0.28-−0.10 (m, 2H). LCMS (ESI, m/z): 333 (fragment peak).

### Example 16

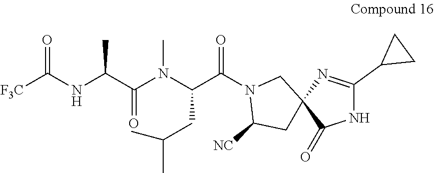 [SMILES: CC(C)C[C@@H](C(=O)N1C[C@@]2(C[C@H]1C#N)N=C(C1CC1)NC2=O)N(C)C(=O)[C@H](C)NC(=O)C(F)(F)F]

Compound 16 was prepared similarly as described for Compound 6 using (5R,8S)-2-cyclopropyl-4-oxo-1,3,7-triazaspiro[4.4]non-1-ene-8-carboxamide HCl in place of (3S,5S)-6-oxo-2,7-diazaspiro[4.4]nonane-3-carboxamide hydrochloride. ¹H NMR (500 MHz, 368K, DMSO-d₆) δ 10.68 (br. s., 1H), 9.20 (br. s., 1H), 5.18 (m, 1H), 4.84 (m, 1H), 4.72 (m, 1H), 3.49-3.68 (m, 2H), 2.92 (s, 3H), 2.41 (m, 1H), 2.32 (m, 1H), 1.60-1.72 (m, 2H), 1.57 (m, 1H), 1.49 (m, 1H), 1.29 (d, 3H), 0.95 (m, 4H), 0.89 (d, 3H), 0.85 (d, 3H). LC-MS (ESI, m/z): 499 [M+H]⁺.

(5R,8S)-2-Cyclopropyl-4-oxo-1,3,7-triazaspiro[4.4]non-1-ene-8-carboxamide HCl was prepared similarly as described for (5R,8S)-4-oxo-2-phenyl-1,3,7-triazaspiro[4.4]non-1-ene-8-carboxamide using cyclopropanecarbonyl chloride in place of benzoyl chloride.

### Example 17

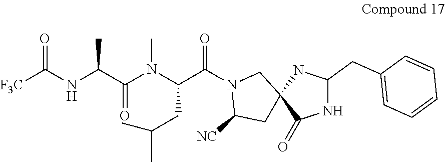

Compound 17 was prepared similarly as described for Compound 6 using (5R,8S)-2-benzyl-4-oxo-1,3,7-triazaspiro[4.4]non-1-ene-8-carboxamide HCl in place of (3S,5S)-6-oxo-2,7-diazaspiro[4.4]nonane-3-carboxamide HCl. ¹H NMR (500 MHz, 365K, DMSO-d₆) δ 10.88 (br. s., 1H), 9.20 (br. s., 1H), 7.21-7.35 (m, 5H), 5.20 (m, 1H), 4.88 (m, 1H), 4.71 (m, 1H), 3.55-3.74 (m, 4H), 2.93 (s, 3H), 2.44 (m, 1H), 2.35 (m, 1H), 1.53-1.72 (m, 2H), 1.48 (m, 1H), 1.25 (d, 3H), 0.89 (d, 3H), 0.84 (d, 3H). LC-MS (ESI, m/z): 549 [M+H]⁺.

(5R,8S)-2-Benzyl-4-oxo-1,3,7-triazaspiro[4.4]non-1-ene-8-carboxamide HCl was prepared similarly as described for (5R,8S)-4-oxo-2-phenyl-1,3,7-triazaspiro[4.4]non-1-ene-8-carboxamide using phenylacetyl chloride instead of benzoyl chloride.

### Example 18

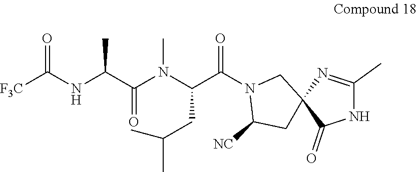 [SMILES: CC1=N[C@@]2(C[C@@H](C#N)N(C(=O)[C@H](CC(C)C)N(C)C(=O)[C@H](C)NC(=O)C(F)(F)F)C2)C(=O)N1]

Compound 18 was prepared similarly as described for Compound 6 using (5R,8S)-2-methyl-4-oxo-1,3,7-triazaspiro[4.4]non-1-ene-8-carboxamide HCl in place of (3S,5S)-6-oxo-2,7-diazaspiro[4.4]nonane-3-carboxamide HCl. ¹H NMR (500 MHz, 367K, DMSO-d₆) δ 10.8 (br. s., 1H), 9.2 (br. s., 1H), 5.20 (m, 1H), 4.85 (m, 1H), 4.73 (m, 1H), 3.53-3.67 (m, 2H), 2.94 (s, 3H), 2.35-2.45 (m, 1H), 2.34 (m, 1H), 2.00 (s, 3H), 1.52-1.70 (m, 2H), 1.49 (m, 1H), 1.27 (d, 3H), 0.88 (d, 3H), 0.85 (d, 3H). LC-MS (ESI, m/z): 473 [M+H]⁺.

(5R,8S)-2-Methyl-4-oxo-1,3,7-triazaspiro[4.4]non-1-ene-8-carboxamide HCl was prepared similarly as described for (5R,8S)-4-oxo-2-phenyl-1,3,7-triazaspiro[4.4]non-1-ene-8-carboxamide using acetyl chloride in place of benzoyl chloride.

### Example 19

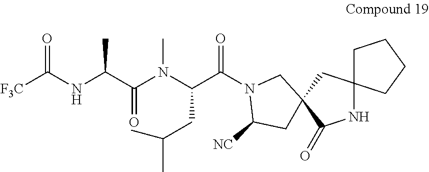 [SMILES: CC(C)C[C@@H](C(=O)N1C[C@@]2(C[C@H]1C#N)CC1(CCCC1)NC2=O)N(C)C(=O)[C@H](C)NC(=O)C(F)(F)F]

Compound 19 was prepared similarly as described for Compound 8 using (3S,5R)-13-oxo-2,12-diazadispiro[4.1.4⁷.2⁵]tridecane-3-carboxamide HCl in place of (3S,5S)-6-oxo-2,7-diazaspiro[4.4]nonane-3-carboxamide HCl. ¹H NMR (500 MHz, 368K, DMSO-d₆) δ 7.92 (s, 1H), 5.17 (m, 1H), 4.69-4.80 (m, 2H), 3.56-3.79 (m, 2H), 2.95 (s, 3H), 2.28-2.42 (m, 2H), 1.88-2.04 (m, 2H), 1.44-1.74 (m, 11H), 1.27 (d, 3H), 0.90 (d, 3H); 0.86 (d, 3H). LC-MS (ESI, m/z): 514 [M+H]⁺.

(3S,5R)-13-Oxo-2,12-diazadispiro[4.1.4⁷.2⁵]tridecane-3-carboxamide HCl: To a solution of 1-(t-butyl) 2,4-diethyl (2S,4R)-4-(cyanomethyl)pyrrolidine-1,2,4-tricarboxylate (2.2 g, 6.21 mmol) in THF (22 mL) were added Zn powder (1.2 g, 18.6 mmol) and allyl bromide (1.6 mL, 18.6 mmol). The mixture was heated at 80° C. for 1 h. After cooling to rt, the mixture was partitioned between 1N HCl and EA. The phases were separated. The aqueous phase was extracted with EA (2×100 mL). The organic phases were combined, washed with brine and dried over Na₂SO₄. The solids were removed by filtration and the filtrate was concentrated under reduced pressure. The residue was purified by flash chromatography on silica gel using a gradient of EA (60 to 70%) in PE to afford 2-(t-butyl) 3-ethyl (3S,5R)-8,8-diallyl-6-oxo-2,7-diazaspiro[4.4]nonane-2,3-dicarboxylate (1.6 g, 65%) as a colorless oil.

A mixture of 2-(t-butyl) 3-ethyl (3S,5R)-8,8-diallyl-6-oxo-2,7-diazaspiro[4.4]nonane-2,3-dicarboxylate (1.5 g, 3.82 mmol) and Grubbs II catalyst (324 mg, 0.382 mmol) in DCM (15 mL) was heated at 35° C. for 90 min. The mixture was concentrated under reduced pressure. The residue was purified by flash chromatography on silica gel using a gradient of EA (60 to 70%) in PE to afford 2-(t-butyl) 3-ethyl (3S,5R)-13-oxo-2,12-diazadispiro[4.1.4⁷.2⁵]tridec-9-ene-2,3-dicarboxylate (1.1 g, 79%) as an off-white solid.

A mixture of 2-(t-butyl) 3-ethyl (3S,5R)-13-oxo-2,12-diazadispiro[4.1.4⁷.2⁵]tridec-9-ene-2,3-dicarboxylate (500 mg, 1.37 mmol) and Pd/C (250 mg) in CH₃OH (10 mL) was stirred at rt for 16 h under H₂ atmosphere. The mixture was filtered through celite and the filtrate were concentrated under reduced pressure. The residue was purified by flash chromatography on silica gel using a gradient of EA (60 to 70%) in PE to afford 2-(t-butyl) 3-ethyl (3S,5R)-13-oxo-2,12-diazadispiro[4.1.4⁷.2⁵]tridecane-2,3-dicarboxylate (450 mg, 89%) as a white solid. ¹H NMR (400 MHz, CDCl₃) δ 5.81 (s, 1H), 4.14-4.26 (m, 3H), 3.47-3.70 (m, 2H), 2.36-2.49 (m, 1H), 2.01-2.17 (m, 3H), 1.60-1.80 (m, 8H), 1.41-1.49 (m, 9H), 1.22-1.31 (m, 3H). LC-MS (ESI, m/z): 367 [M+H]⁺.

To a solution of 2-(t-butyl) 3-ethyl (3S,5R)-13-oxo-2,12-diazadispiro[4.1.4⁷.2⁵]tridecane-2,3-dicarboxylate (450 mg, 1.22 mmol) in THF (2.5 mL) and water (2.5 mL) cooled at 0° C. was added LiOH (77 mg, 1.84 mmol). The mixture was stirred at 0° C. for 1 h and then at rt for 5 h. The mixture was partially concentrated under reduced pressure to remove the organic solvent. The residue was acidified with 1N HCl until pH ˜3. The precipitate was filtered, washed with water and dried under high vacuum to afford (3S,5R)-2-(t-butoxycarbonyl)-13-oxo-2,12-diazadispiro[4.1.4⁷.2⁵]tridecane-3-carboxylic acid (350 mg, 84%) as an off-white solid.

To a solution of (3S,5R)-2-(t-butoxycarbonyl)-13-oxo-2,12-diazadispiro[4.1.4⁷.2⁵]tridecane-3-carboxylic acid (350 mg, 1.03 mmol) in DMF (3.5 mL) cooled at 0° C. were added EDC·HCl (395 mg, 2.07 mmol, 2.0 eq.), HOAt (140 mg, 1.03 mmol), NEt₃ (0.43 mL, 3.11 mmol) and NH₄Cl (274 mg, 5.18 mmol). The mixture was stirred at rt for 16 h. The mixture was diluted with water (5 mL) and extracted with 20% CH₃OH:DCM (3×10 mL). The organic phases were combined and dried over Na₂SO₄. The solids were removed by filtration and the filtrate was concentrated under reduced pressure. The residue was purified by flash chromatography on silica gel using a gradient of CH₃OH (20 to 30%) in DCM to afford t-butyl (3S,5R)-3-carbamoyl-13-oxo-2,12-diazadispiro[4.1.4⁷.2⁵]tridecane-2-carboxylate (300 mg, 86%) as an off-white solid.

To a solution of t-butyl (3S,5R)-3-carbamoyl-13-oxo-2,12-diazadispiro[4.1.4⁷.2⁵]tridecane-2-carboxylate (150 mg, 0.445 mmol) in DCM (1 mL) cooled at 0° C. was added 4M HCl in 1,4-dioxane (0.55 mL, 2.20 mmol). The mixture was stirred at rt for 3 h. The mixture was concentrated under reduced pressure and co-evaporated with Et₂O to afford quantitatively (3S,5R)-13-oxo-2,12-diazadispiro[4.1.4⁷.2⁵]tridecane-3-carboxamide HCl as a white solid. LC-MS (ESI, m/z): 238 [M+H]⁺.

### Example 20

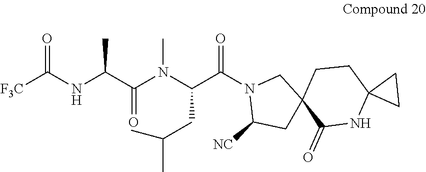 [SMILES: CC(C)C[C@@H](C(=O)N1C[C@]2(CCC3(CC3)NC2=O)C[C@H]1C#N)N(C)C(=O)[C@H](C)NC(=O)C(F)(F)F]

Compound 20 was prepared similarly as described for Compound 13 using 1-(t-butyl) 2,4-diethyl (2S,4S)-4-(2-cyanoethyl)pyrrolidine-1,2,4-tricarboxylate in place of 1-(t-butyl) 2,4-diethyl (2S,4R)-4-(cyanomethyl)pyrrolidine-1,2,4-tricarboxylate. ¹H NMR (500 MHz, 368K, DMSO-d₆) δ 9.23 (br. s., 1H), 7.44 (s, 1H), 5.21 (m, 1H), 4.72-4.84 (m, 2H), 3.66-3.82 (m, 2H), 2.97 (s, 3H), 2.53 (m, 1H), 2.40 (m, 1H), 1.54-1.77 (m, 6H), 1.50 (m, 1H), 1.30 (d, 3H), 0.90 (d, 3H), 0.86 (d, 3H), 0.74 (m, 2H), 0.58 (m, 2H). LC-MS (ESI, m/z): 500 [M+H]⁺.

### Example 21

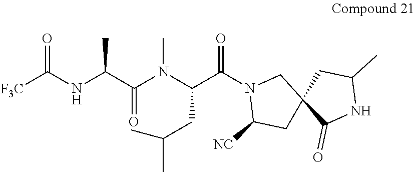

To a solution of 1-(t-butyl) 2-methyl (S)-5-oxopyrrolidine-1,2-dicarboxylate (10 g, 41.2 mmol) in THF (100 mL) cooled at −78° C. was added 1M LiHMDS in THF (82 mL, 82.3 mmol). The mixture was stirred at −78° C. for 1 h and ethyl chloroformate (4.67 mL, 49.3 mmol) was added. The mixture was stirred at −78° C. for 1 h and was allowed to warm to rt. The reaction was quenched by addition of NH₄Cl (sat., aq.). The mixture was extracted with EA (2×100 mL). The organic phases were combined, washed with brine (2×50 mL) and dried over Na₂SO₄. The solids were removed by filtration and the filtrate was concentrated under reduced pressure. The residue was purified by flash chromatography on silica gel using a gradient of EA (20 to 25%) in PE to afford 1-(t-butyl) 4-ethyl 2-methyl (2S)-5-oxopyrrolidine-1,2,4-tricarboxylate (11 g, 85%) as a colorless oil.

To a solution of 1-(t-butyl) 4-ethyl 2-methyl (2S)-5-oxopyrrolidine-1,2,4-tricarboxylate (5 g, 15.9 mmol) in THF (50 mL) cooled at 0° C. was added portionwise NaH (762 mg, 19.0 mmol). The mixture was stirred at 0° C. for 30 min and 3-bromo-2-methylprop-1-ene (2.41 mL, 23.8 mmol) was added. The mixture was stirred at rt for 16 h. The reaction was quenched by addition of NH₄Cl (sat., aq. 50 mL). The mixture was extracted with EA (3×50 mL). The organic phases were combined, washed with brine (2×50 mL) and dried over Na₂SO₄. The solids were removed by filtration and the filtrate was concentrated under reduced pressure. The residue was triturated with 10% Et₂O in pentane (25 mL) to afford 1-(t-butyl) 4-ethyl 2-methyl (2S,4S)-4-(2-methylallyl)-5-oxopyrrolidine-1,2,4-tricarboxylate (1.3 g, 22%) as an off-white solid. ¹H NMR (400 MHz, CDCl₃) δ 4.86 (m, 1H), 4.72 (m, 1H), 4.59 (dd, 1H), 4.17 (q, 2H), 3.71 (s, 3H), 2.96 (d, 1H), 2.86 (dd, 1H), 2.41 (d, 1H), 2.33 (dd, 1H), 1.66 (s, 3H), 1.49 (s, 9H), 1.26 (t, 3H).

To a solution of 1-(t-butyl) 4-ethyl 2-methyl (2S,4S)-4-(2-methylallyl)-5-oxopyrrolidine-1,2,4-tricarboxylate (500 mg, 1.35 mmol) in THF (5 mL) cooled at −78° C. was added dropwise 1M lithium triethyl borohydride solution in THF (1.5 mL, 1.50 mmol). The mixture was stirred at −78° C. for 1 h. The reaction was quenched by the addition of sat. NaHCO₃ (5 mL) and hydrogen peroxide (0.3 mL). The mixture was extracted with EA (3×20 mL). The organic phases were combined, washed with brine (10 mL) and dried over Na₂SO₄. The solids were removed by filtration and the filtrate was concentrated under reduced pressure.

The residue (500 mg) was dissolved in DCM (5 mL) and the mixture was cooled at −78° C. Triethylsilane (0.21 mL, 1.34 mmol) and BF₃·OEt₂ (0.2 mL, 1.48 mmol) were added. The mixture was stirred at −78° C. for 30 min. Triethylsilane (0.21 mL, 1.34 mmol) and BF₃·OEt₂ (0.2 mL, 1.48 mmol) were added a second time. The mixture was allowed to warm to rt over 1 h. The mixture was diluted with DCM (10 mL) and washed with sat. NaHCO₃ (5 mL). The phases were separated. The aqueous phase was extracted with DCM (2×10 mL). The organic phases were combined, washed with brine (10 mL) and dried over Na₂SO₄. The solids were removed by filtration and the filtrate was concentrated under reduced pressure.

The residue (300 mg) was dissolved in DCM (6 mL). NEt₃ (0.49 mL, 3.52 mmol) and BOC₂O (0.386 g, 1.77 mmol) were added and the mixture was stirred at rt for 2 h. The mixture was concentrated under reduced pressure. The residue was purified by flash chromatography on silica gel using a gradient of EA (25 to 30%) in PE to afford 1-(t-butyl) 4-ethyl 2-methyl (2S,4S)-4-(2-methylallyl)pyrrolidine-1,2,4-tricarboxylate (300 mg, 62% over 3 steps) as a colorless oil.

To a stirred solution of 1-(t-butyl) 4-ethyl 2-methyl (2S,4S)-4-(2-methylallyl)pyrrolidine-1,2,4-tricarboxylate (1.7 g, 4.78 mmol) in dioxane (17 mL) at rt were added 4% OsO₄ in water (1 mL, 0.157 mmol) and a solution of NaIO₄ (2 g, 9.57 mmol) in water (20 mL). The mixture was stirred at rt for 3 h. The mixture was diluted with water (20 mL) and extracted with EA (2×30 mL). The organic phases were combined and dried over Na₂SO₄. The solids were removed by filtration and the filtrate was concentrated under reduced pressure. The residue was purified by flash chromatography on silica gel using a gradient of EA (30 to 35%) in PE to afford 1-(t-butyl) 4-ethyl 2-methyl (2S,4R)-4-(2-oxopropyl)pyrrolidine-1,2,4-tricarboxylate (1.5 g, 88%) as a colorless oil.

To a solution of 1-(t-butyl) 4-ethyl 2-methyl (2S,4R)-4-(2-oxopropyl)pyrrolidine-1,2,4-tricarboxylate (1.5 g, 4.20 mmol) in CH₃OH (15 mL) cooled at 0° C. was added NH₄OAc (1.61 g, 21.0 mmol). The mixture was stirred at 0° C. for 10 min and NaBH₃CN (1.32 g, 21.0 mmol) was added. The mixture was refluxed for 4 h. The mixture was concentrated under reduced pressure. The residue was partitioned between water (30 mL) and 10% CH₃OH in DCM (10 mL). The aqueous phase was extracted with 10% CH₃OH in DCM (2×10 mL). The organic phases were combined and dried over Na₂SO₄. The solids were removed by filtration and the filtrate was concentrated under reduced pressure to afford 2-(t-butyl) 3-methyl (3S,5S)-8-methyl-6-oxo-2,7-diazaspiro[4.4]nonane-2,3-dicarboxylate (830 mg, 63%) as an off-white solid.

A solution of 2-(t-butyl) 3-methyl (3S,5S)-8-methyl-6-oxo-2,7-diazaspiro[4.4]nonane-2,3-dicarboxylate (810 mg, 2.59 mmol) in methanol (20 mL) cooled at 0° C. was purged with ammonia gas for 10 min. The resulting solution was stirred at 60° C. for 48 h in a steel bomb. The mixture was concentrated under reduced pressure to afford t-butyl (3S,5S)-3-carbamoyl-8-methyl-6-oxo-2,7-diazaspiro[4.4]nonane-2-carboxylate (550 mg, 71%) as an off-white solid.

To a solution of t-butyl (3S,5S)-3-carbamoyl-8-methyl-6-oxo-2,7-diazaspiro[4.4]nonane-2-carboxylate (150 mg, 0.505 mmol) in DCM (1.5 mL) cooled at 0° C. was added 4M HCl in dioxane (0.63 mL, 2.52 mmol). The mixture was stirred at rt for 3 h. The mixture was concentrated under reduced pressure and co-evaporated with Et₂O to afford quantitatively (3S,5S)-8-methyl-6-oxo-2,7-diazaspiro[4.4]nonane-3-carboxamide HCl as a white solid.

To a solution of N-methyl-N-((2,2,2-trifluoroacetyl)-L-alanyl)-L-leucine (150 mg, 0.480 mmol) in DMF (1.5 mL) cooled at 0° C. were (3S,5S)-8-methyl-6-oxo-2,7-diazaspiro[4.4]nonane-3-carboxamide HCl (113 mg, 0.480 mmol), EDC·HCl (184 mg, 0.960 mmol), HOAt (65 mg, 0.478 mmol) and NEt₃ (0.2 mL, 1.44 mmol). The mixture was stirred at rt for 16 h. The mixture was diluted with water (5 mL) and extracted with 20% CH₃OH in DCM (10 mL). The phases were separated. The aqueous phase was extracted with 20% CH₃OH in DCM (2×10 mL). The organic phases were combined and dried over Na₂SO₄. The solids were removed by filtration and the filtrate was concentrated under reduced pressure. The residue was purified by flash chromatography on C18 using a gradient of ACN (20 to 30%) in 0.1% FA in water to afford (3S,5S)-8-methyl-2-(N-methyl-N-((2,2,2-trifluoroacetyl)-L-alanyl)-L-leucyl)-6-oxo-2,7-diazaspiro[4.4]nonane-3-carboxamide (100 mg, 42%) as an off-white solid.

To a solution of (3S,5S)-8-methyl-2-(N-methyl-N-((2,2,2-trifluoroacetyl)-L-alanyl)-L-leucyl)-6-oxo-2,7-diazaspiro[4.4]nonane-3-carboxamide (90 mg, 0.183 mmol) in DCM (1 mL) cooled at 0° C. were added pyridine (0.032 mL, 0.402 mmol) and TFAA (0.028 mL, 0.201 mmol). The mixture was stirred at 0° C. for 1 h. The reaction mixture was diluted with water (5 mL) and extracted with DCM (10 mL). The phases were separated. The aqueous phase was extracted with DCM (2×10 mL). The organic phases were combined, washed with brine (2×5 mL) and dried over Na₂SO₄. The solids were removed by filtration and the filtrate was concentrated under reduced pressure. The residue was purified by prep-HPLC (Column: X-SELECT-C18, 19×250 mm, 5 m; Mobile Phase A: 10 mM NH₄HCO₃ in water, Mobile Phase B: ACN; Flow rate: 10 mL/min; Gradient: 25 to 60% B in 9 min) to afford (2S)—N-((2S)-1-((3S,5S)-3-cyano-8-methyl-6-oxo-2,7-diazaspiro[4.4]nonan-2-yl)-4-methyl-1-oxopentan-2-yl)-N-methyl-2-(2,2,2-trifluoroacetamido)propanamide (52 mg, 54%) as a white solid. 1H NMR (500 MHz, 363K, DMSO-d₆) δ 9.25 (br. s., 1H), 7.75 (m, 1H), 5.19 (m, 1H), 4.75 (m, 2H), 3.39-3.82 (m, 3H), 2.97 (s, 3H), 2.00-2.47 (m, 3H), 1.61 (m, 2H), 1.40-1.56 (m, 2H), 1.31 (d, 3H), 1.13 (m, 3H), 0.81-0.94 (m, 6H). LC-MS (ESI, m/z): 474 [M+H]⁺.

### Example 22

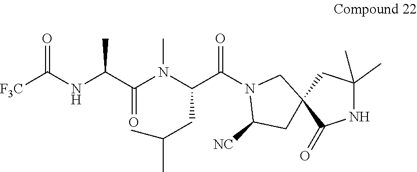 [SMILES: CC(C)C[C@@H](C(=O)N1C[C@@]2(C[C@H]1C#N)CC(C)(C)NC2=O)N(C)C(=O)[C@H](C)NC(=O)C(F)(F)F]

Compound 22 was prepared similarly as described for Compound 8 using 2-(t-butyl) 3-ethyl (3S,5R)-8,8-dimethyl-6-oxo-2,7-diazaspiro[4.4]nonane-2,3-dicarboxylate in place of 2-(t-butyl) 3-ethyl (3S,5S)-6-oxo-2,7-diazaspiro[4.4]nonane-2,3-dicarboxylate. ¹H NMR (500 MHz, 368K, DMSO-d₆) δ 9.21 (br. s., 1H), 7.78 (s, 1H), 5.17 (m, 1H), 4.76 (m, 2H), 3.58-3.73 (m, 2H), 2.98 (s, 3H), 2.29-2.46 (m, 2H), 1.77-1.93 (m, 2H), 1.56-1.69 (m, 2H), 1.50 (m, 1H), 1.31 (d, 3H), 1.21 (d, 6H), 0.90 (d, 3H), 0.86 (d, 3H). LC-MS (ESI, m/z): 488 [M+H]⁺.

2-(t-Butyl) 3-ethyl (3S,5R)-8,8-dimethyl-6-oxo-2,7-diazaspiro[4.4]nonane-2,3-dicarboxylate: A mixture of 7-(t-butyl) 8-ethyl (5R,8S)-10-oxo-7,11-diazadispiro[2.1.4⁵.2³]undecane-7,8-dicarboxylate (1.6 g, 4.73 mmol) and PtO₂ (320 mg) in acetic acid (16 mL) was stirred at rt for 4 h under H₂ atmosphere. The mixture was filtered through celite and washed with EA (100 mL). The filtrate was concentrated under reduced pressure. The residue was purified by prep-HPLC (Column: HICHROM 5 C18, 25×150 mm, m; Mobile Phase A: 0.1% FA in water, Mobile Phase B: ACN; Flow rate: 22 mL/min; Gradient: 25% B to 55% B in 12 min) to afford 2-(t-butyl) 3-ethyl (3S,5R)-8,8-dimethyl-6-oxo-2,7-diazaspiro[4.4]nonane-2,3-dicarboxylate (300 mg, 18%) as an off-white solid. ¹H NMR (400 MHz, DMSO-d₆) δ 8.06 (br. s., 1H), 4.04-4.24 (m, 3H), 3.38-3.48 (m, 1H), 3.33 (m, 1H), 2.21 (m, 1H), 2.08 (m, 1H), 1.80-2.02 (m, 2H), 1.30-1.40 (m, 9H), 1.12-1.25 (m, 9H).

2-(t-Butyl) 3-ethyl (3S,5S)-8-ethyl-6-oxo-2,7-diazaspiro[4.4]nonane-2,3-dicarboxylate (1 g, 62%) was isolated during the prep-HPLC purification. ¹H NMR (400 MHz, DMSO-d₆) δ 8.05-8.12 (m, 1H), 4.02-4.26 (m, 3H), 3.40-3.52 (m, 2H), 3.38 (m, 1H), 2.22-2.32 (m, 2H), 2.11 (m, 1H), 1.76-1.88 (m, 1H), 1.51 (m, 2H), 1.30-1.40 (m, 9H), 1.15-1.22 (m, 3H), 0.86 (t, 3H).

### Example 23

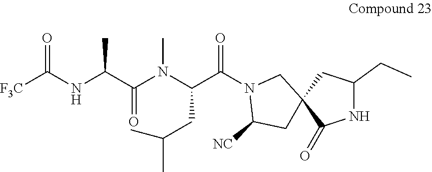

Compound 23 was prepared similarly as described for Compound 8 using 2-(t-butyl) 3-ethyl (3S,5S)-8-ethyl-6-oxo-2,7-diazaspiro[4.4]nonane-2,3-dicarboxylate in place of 2-(t-butyl) 3-ethyl (3S,5S)-6-oxo-2,7-diazaspiro[4.4]nonane-2,3-dicarboxylate. ¹H NMR (500 MHz, 368K, DMSO-d₆) δ 9.21 (br. s., 1H), 7.80 (m, 1H), 5.17 (m, 1H), 4.77 (m, 2H), 3.59-3.82 (m, 2H), 3.34-3.48 (m, 1H), 2.98 (s, 3H), 2.28-2.46 (m, 2H), 2.18 (m, 1H), 1.60 (m, 2H), 1.44-1.57 (m, 3H), 1.39 (m, 1H), 1.30 (d, 3H), 0.83-0.93 (m, 9H). LC-MS (ESI, m/z): 488 [M+H]⁺.

### Example 24

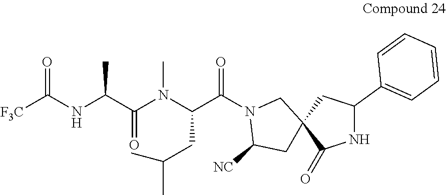

Compound 24 was prepared similarly as described for Compound 8 using t-butyl (3S,5S)-3-carbamoyl-6-oxo-8-phenyl-2,7-diazaspiro[4.4]nonane-2-carboxylate in place of t-butyl (3S,5S)-3-carbamoyl-6-oxo-2,7-diazaspiro[4.4]nonane-2-carboxylate. ¹H NMR (500 MHz, 368K, DMSO-d₆) δ 9.26 (br. s., 1H), 8.23 (m, 1H), 7.24-7.38 (m, 5H), 5.20 (m, 1H), 4.79 (m, 1H), 4.61-4.75 (m, 2H), 3.73-3.96 (m, 1H), 3.49-3.62 (m, 1H), 2.88-3.04 (m, 3H), 2.51-2.63 (m, 1H), 2.21-2.45 (m, 2H), 1.69-1.86 (m, 1H), 1.60 (m, 2H), 1.50 (m, 1H), 1.10-1.38 (m, 3H), 0.77-0.92 (m, 6H). LC-MS (ESI, m/z): 534 [M−H]⁻.

t-Butyl (3S,5S)-3-carbamoyl-6-oxo-8-phenyl-2,7-diazaspiro[4.4]nonane-2-carboxylate: To a solution of 1-(t-butyl) 2-methyl (S)-4-oxopyrrolidine-1,2-dicarboxylate (10 g, 41.2 mmol) and bromoform (10 g, 82.2 mmol) in THF (100 mL) cooled to −78° C. was added 1M LiHMDS in THF (82.2 mL, 82.2 mmol). The mixture was stirred at −78° C. for 1 h. The reaction was quenched by the addition of sat. NH₄Cl(50 mL). The mixture was extracted with EA (2×100 mL). The organic phases were combined, washed with brine (2×75 mL) and dried over Na₂SO₄. The solids were removed by filtration and the filtrate was concentrated under reduced pressure. The residue was purified by flash chromatography on silica gel using a gradient of EA (30 to 50%) in PE to afford 1-(t-butyl) 2-methyl (2S,4S)-4-hydroxy-4-(tribromomethyl)pyrrolidine-1,2-dicarboxylate (11 g, 55%) as a beige solid.

To a solution of 1-(t-butyl) 2-methyl (2S,4S)-4-hydroxy-4-(tribromomethyl)pyrrolidine-1,2-dicarboxylate (10 g, 20.3 mmol) in dioxane (100 mL) cooled at 0° C. were added CH₃OH (2.4 mL, 60.9 mmol) and DBU (6.64 mL, 44.6 mmol). The mixture was allowed to warm to rt and then stirred at rt for 1 h. The reaction was quenched by the addition of sat. NH₄Cl (30 mL). The mixture was extracted with EA (2×50 mL). The organic phases were combined, washed with brine (2×40 mL) and dried over Na₂SO₄. The solids were removed by filtration and the filtrate was concentrated under reduced pressure. The residue was purified by flash chromatography on silica gel using a gradient of EA (30 to 50%) in PE to afford 1-(t-butyl) 2,4-dimethyl (2S,4R)-4-bromopyrrolidine-1,2,4-tricarboxylate (4.0 g, 54%) as a brown oil.

To a solution of 1-(t-butyl) 2,4-dimethyl (2S,4R)-4-bromopyrrolidine-1,2,4-tricarboxylate (4.0 g, 11.0 mmol) in dioxane (40 mL) were added t-butyldimethyl((1-phenylvinyl)oxy)silane (3.06 g, 13.1 mmol), NaHCO₃ (1.84 g, 21.9 mmol), CuI (20 mg, 0.109 mmol) and N,N,N′,N″,N″-pentamethyldiethylenetriamine (0.04 mL, 0.219 mmol). The mixture was heated at 85° C. for 30 h. After cooling to rt, the mixture was diluted with water (20 mL) and extracted with EA (2×30 mL). The organic phases were combined, washed with brine (2×20 mL) and dried over Na₂SO₄. The solids were removed by filtration and the filtrate was concentrated under reduced pressure. The residue was purified by flash chromatography on silica gel using a gradient of EA (15 to 30%) in PE to afford 1-(t-butyl) 2,4-dimethyl (2S)-4-(2-((t-butyldimethylsilyl)oxy)-2-phenylvinyl)pyrrolidine-1,2,4-tricarboxylate (4.0 g, 70%) as a yellow oil.

To a solution of 1-(t-butyl) 2,4-dimethyl (2S)-4-(2-((t-butyldimethylsilyl)oxy)-2-phenylvinyl)pyrrolidine-1,2,4-tricarboxylate (4.0 g, 7.70 mmol) in THF (40 mL) cooled at 0° C. was added 1M TBAF in THF (3.8 mL, 3.8 mmol) and the mixture was stirred at 0° C. for 5 min. The mixture was diluted with ice/water (15 mL) and extracted with EA (2×20 mL). The organic phases were combined, washed with brine (2×15 mL) and dried over Na₂SO₄. The solids were removed by filtration and the filtrate was concentrated under reduced pressure. The residue was purified by flash chromatography on silica gel using a gradient of EA (15 to 30%) in PE to afford 1-(t-butyl) 2,4-dimethyl (2S)-4-(2-oxo-2-phenylethyl)pyrrolidine-1,2,4-tricarboxylate (2.5 g, 80%) as a colorless oil.

1-(t-Butyl) 2,4-dimethyl (2S)-4-(2-oxo-2-phenylethyl)pyrrolidine-1,2,4-tricarboxylate (2.0 g) was purified by prep-SFC using the following conditions: Column: Chiralpak IC, 4.6×250 cm, 5 m; Mobile Phase A: CO₂, Mobile Phase B: ACN; Flow rate: 3 g/min; Elution condition: isocratic 40% B; Column Temperature: 30° C.; Purification resulted in 1-(t-Butyl) 2,4-dimethyl (2S,4R)-4-(2-oxo-2-phenylethyl)pyrrolidine-1,2,4-tricarboxylate.

¹H NMR (400 MHz, CDCl₃) δ 7.93 (d, 2H), 7.59 (m, 1H), 7.45 (m, 2H), 4.28-4.43 (m, 1H), 3.90 (m, 1H), 3.68 (m, 6H), 3.35-3.61 (m, 3H), 2.59-2.74 (m, 1H), 2.31-2.43 (m, 1H), 1.43 (m, 9H).

To a solution of 1-(t-butyl) 2,4-dimethyl (2S,4R)-4-(2-oxo-2-phenylethyl)pyrrolidine-1,2,4-tricarboxylate (600 mg, 1.48 mmol) in CH₃OH (40 mL) was added NH₄OAc (570 mg, 7.41 mmol). The mixture was stirred at rt for 2 h. NaBH₃CN was added (465 mg, 7.41 mmol) and the mixture was heated at 60° C. for 16 h in a sealed tube. After cooling to rt, NH₄OAc (570 mg, 7.41 mmol) and NaBH₃CN (465 mg, 7.41 mmol) were added a second time. The mixture was heated at 60° C. for 16 h in a sealed tube. The mixture was concentrated under reduced pressure. The residue was partitioned between water (10 mL) and EA (10 mL). The aqueous phase was extracted with EA (10 mL). The organic phases were combined, washed with brine (2×15 mL) and dried over Na₂SO₄. The solids were removed by filtration and the filtrate was concentrated under reduced pressure. The residue was purified by flash chromatography on silica gel using a gradient of EA (60 to 100%) in PE to afford 2-(t-butyl) 3-methyl (3S,5S)-6-oxo-8-phenyl-2,7-diazaspiro[4.4]nonane-2,3-dicarboxylate (400 mg, 72%) as an off-white solid.

A solution of 2-(t-butyl) 3-methyl (3S,5S)-6-oxo-8-phenyl-2,7-diazaspiro[4.4]nonane-2,3-dicarboxylate (400 mg, 1.07 mmol) in CH₃OH (5 mL) cooled at −78° C. was purged with ammonia gas for 15 min. The mixture was heated in a steel bomb at 60° C. for 24 h. The mixture was concentrated under reduced pressure. The residue was purified by flash chromatography on silica gel using a gradient of CH₃OH (1 to 10%) in DCM to afford t-butyl (3S,5S)-3-carbamoyl-6-oxo-8-phenyl-2,7-diazaspiro[4.4]nonane-2-carboxylate (350 mg, 92%) as a white solid.

### Example 25

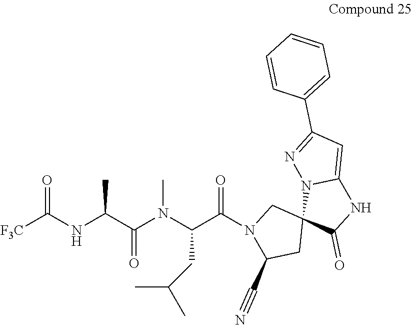 [SMILES: CC(C)C[C@@H](C(=O)N1C[C@]2(C[C@H]1C#N)C(=O)Nc1cc(-c3ccccc3)nn12)N(C)C(=O)[C@H](C)NC(=O)C(F)(F)F]

To a mixture of t-butyl (3R,5'S)-6-bromo-5′-carbamoyl-2-oxo-1H-spiro[pyrazolo[1,5-a]imidazole-3,3′-pyrrolidine]-1′-carboxylate (300 mg, 0.750 mmol), potassium phosphate (318 mg, 1.50 mmol) and phenyl boronic acid (110 mg, 0.900 mmol) in dioxane (5 mL)/H₂O (1 mL) was added [AMPhos-PdCl₂]₂ (67.0 mg, 0.075 mmol). The mixture was stirred for 2 h at 90° C. under nitrogen and the reaction was quenched with water (10 mL). The mixture was extracted with EA (3×10 mL). The organic layers were combined, washed with brine (2×10 mL) and dried over anhydrous sodium sulfate. The solids were removed by filtration and the filtrate was concentrated under reduced. The crude product was purified by TLC (Mobile phase: CH₃OH:DCM=1:12; Rf=0.4; detection: UV) to provide t-butyl (3R,5'S)-5′-carbamoyl-2-oxo-6-phenyl-1H-spiro[pyrazolo[1,5-a]imidazole-3,3′-pyrrolidine]-1′-carboxylate (76.0 mg, 24%) as a yellow solid. LC-MS (ESI, m/z): 298 [M-100+H]⁺.

A mixture of t-butyl (3R,5'S)-5′-carbamoyl-2-oxo-6-phenyl-1H-spiro[pyrazolo[1,5-a]imidazole-3,3′-pyrrolidine]-1′-carboxylate (100 mg, 0.252 mmol) in HCl (3 mL, 4 M in dioxane) was stirred for 2 h at rt. The mixture was concentrated under reduced pressure to afford (3R,5'S)-2-oxo-6-phenyl-1H-spiro[pyrazolo[1,5-a]imidazole-3,3′-pyrrolidine]-5′-carboxamide (75 mg, crude). LC-MS (ESI, m/z): 298 [M+H]⁺.

To a mixture of (3R,5'S)-2-oxo-6-phenyl-1H-spiro[pyrazolo[1,5-a]imidazole-3,3′-pyrrolidine]-5′-carboxamide (75.0 mg, 0.252 mmol), (2S)-4-methyl-2-[(2S)—N-methyl-2-(2,2,2-trifluoroacetamido) propanamido]pentatonic acid (158 mg, 0.504 mmol) and HATU (192 mg, 0.504 mmol) in DMF (5 mL) was added N-ethyl-N-isopropylpropan-2-amine (325 mg, 2.52 mmol) at −15° C. The mixture was stirred for 30 mins at −15° C. The reaction was quenched with water (10 mL). The mixture was extracted with EA (3×10 mL). The organic layers were combined, washed with brine (2×10 mL) and dried over anhydrous sodium sulfate. The solids were removed by filtration and the filtrate was concentrated under reduced pressure to remove the solvent. The residue was purified by TLC (Mobile phase: CH₃OH:DCM=1:10; Rf=0.4; detection: UV) to provide (3R,5'S)-1′-[(2S)-4-methyl-2-[(2S)—N-methyl-2-(2,2,2-trifluoroacetamido)propanamido]pentanoyl]-2-oxo-6-phenyl-1H-spiro[pyrazolo[1,5-a]imidazole-3,3′-pyrrolidine]-5′-carboxamide (55 mg, crude, isomer ratio:6/4). LC-MS (ESI, m/z): 592 [M+H]⁺.

To a mixture of (3R,5'S)-1′-[(2S)-4-methyl-2-[(2S)—N-methyl-2-(2,2,2-trifluoroacetamido)propanamido]pentanoyl]-2-oxo-6-phenyl-1H-spiro[pyrazolo[1,5-a]imidazole-3,3′-pyrrolidine]-5′-carboxamide (40.0 mg, 0.068 mmol) in DCM (1 mL) were added pyridine (24.0 mg, 0.306 mmol) and trifluoroacetic anhydride (28.0 mg, 0.136 mmol, 2.0 eq.). The mixture was stirred for 2 h at rt and the reaction was quenched with water (10 mL). The mixture was extracted with DCM (3×20 mL). The organic layers were combined, washed with brine (2×10 mL) and dried over anhydrous sodium sulfate. The solids were removed by filtration and the filtrate was concentrated under reduced pressure. The crude product was purified by prep-HPLC (Column: YMC-Actus Triart C 18ExRS, 30×150 mm, 5 m; Mobile Phase A: Water (10 mmol/L NH₄HCO₃+0.05% NH₃H₂O), Mobile Phase B: ACN; Flow rate: 60 mL/min; Gradient: 26% B to 56% B in 8 min; Wave Length: 254 nm/220 nm) to provide (S)—N—((S)-1-((3R,5'S)-5′-cyano-2-oxo-6-phenyl-1,2-dihydrospiro[imidazo[1,2-b]pyrazole-3,3′-pyrrolidin]-1′-yl)-4-methyl-1-oxopentan-2-yl)-N-methyl-2-(2,2,2-trifluoroacetamido)propanamide (9.9 mg, 25%) as a white solid. ¹H NMR (400 MHz, 80° C., DMSO-d₆) δ 9.14 (br, 1H), 7.60-7.78 (m, 2H), 7.25-7.40 (m, 2H), 7.15-7.24 (m, 1H), 6.08 (s, 1H), 4.95-5.40 (m, 2H), 4.30-4.50 (m, 1H), 3.65-4.10 (m, 2H), 2.70-2.90 (m, 4H), 2.55-2.69 (m, 1H), 1.45-1.70 (m, 2H), 1.30-1.44 (m, 1H), 0.50-1.00 (m, 9H). LC-MS (ESI, m/z): 574 [M+H]⁺.

### Example 26

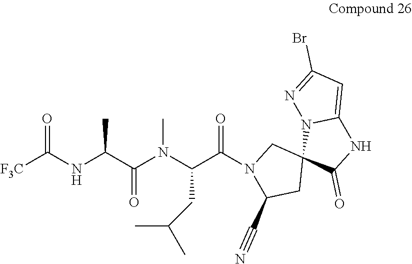 [SMILES: CC(C)C[C@@H](C(=O)N1C[C@]2(C[C@H]1C#N)C(=O)Nc1cc(Br)nn12)N(C)C(=O)[C@H](C)NC(=O)C(F)(F)F]

A mixture of t-butyl (3R,5'S)-6-bromo-5′-carbamoyl-2-oxo-1H-spiro[pyrazolo[1,5-a]imidazole-3,3′-pyrrolidine]-1′-carboxylate (240 mg, 0.600 mmol) in hydrogen chloride (10 ml, 4 M in dioxane) was stirred for 1 h at rt. The mixture was concentrated under reduced pressure to afford (3R,5'S)-6-bromo-2-oxo-1H-spiro[pyrazolo[1,5-a]imidazole-3,3′-pyrrolidine]-5′-carboxamide (180 mg, crude) as a white solid. LC-MS (ESI, m/z): 300 [M+H]⁺.

To a mixture of (3R,5'S)-6-bromo-2-oxo-1H-spiro[pyrazolo[1,5-a]imidazole-3,3′-pyrrolidine]-5′-carboxamide (180 mg, 0.601 mmol), (2S)-4-methyl-2-[(2S)—N-methyl-2-(2,2,2-trifluoroacetamido)propanamido]pentanoic acid (169 mg, 0.541 mmol) and HATU (274 mg, 0.721 mmol) in DMF (7 mL) was added N-ethyl-N-isopropylpropan-2-amine (116 mg, 0.902 mmol) at −15° C. The mixture was stirred for 40 mins at −15° C. and the reaction was quenched with water (15 mL). The mixture was extracted with EA (3×15 mL). The organic layers were combined, washed with brine (2×10 mL) and dried over anhydrous sodium sulfate. The solids were removed by filtration and the filtrate was concentrated under reduced pressure. The crude product was chromatographed on a silica gel column with CH₃OH:DCM (1:11) to provide (3R,5'S)-6-bromo-1′-(N-methyl-N-((2,2,2-trifluoroacetyl)-L-alanyl)leucyl)-2-oxo-1,2-dihydrospiro[imidazo[1,2-b]pyrazole-3,3′-pyrrolidine]-5′-carboxamide (140 mg, 39%) as a white solid. LC-MS (ESI, m/z): 594 [M+H]⁺.

To a mixture of (3R,5'S)-6-bromo-1′-(N-methyl-N-((2,2,2-trifluoroacetyl)-L-alanyl)leucyl)-2-oxo-1,2-dihydrospiro[imidazo[1,2-b]pyrazole-3,3′-pyrrolidine]-5′-carboxamide (140 mg, 0.236 mmol) in DCM (2 mL) were added pyridine (93.0 mg, 1.18 mmol, 5.0 eq.) and trifluoroacetic anhydride (89.0 mg, 0.425 mmol) at 0° C. The mixture was stirred for 1 h at rt and the reaction was quenched with water (5 mL). The mixture was extracted with DCM (3×5 mL). The organic layers were combined, washed with brine (2×5 mL) and dried over anhydrous sodium sulfate. The solids were removed by filtration and the filtrate was concentrated under reduced pressure. The crude product was purified by prep-HPLC (Column: YMC-Actus Triart C18ExRS, 30×150 mm, 5 m; Mobile Phase A: Water (10 mmol/L NH₄HCO₃+0.05% NH₃H₂O), Mobile Phase B: ACN; Flow rate: 60 mL/min; Gradient: 5% B to 20% B in 8 min; Wave Length: 220 nm) to provide (2S)—N-[(2S)-1-[(3R,5'S)-6-bromo-5′-cyano-2-oxo-1H-spiro[pyrazolo[1,5-a]imidazole-3,3′-pyrrolidin]-1′-yl]-4-methyl-1-oxopentan-2-yl]-N-methyl-2-(2,2,2-trifluoroacetamido)propanamide (45.0 mg, 35%) as a white solid. ¹H NMR (400 MHz, DMSO-d₆) δ 10.85-12.30 (m, 1H), 9.33-10.03 (m, 1H), 5.94 (s, 1H), 5.16-5.26 (m, 1H), 4.97-5.08 (m, 1H), 4.55-4.69 (m, 1H), 3.93-4.05 (m, 1H), 3.72-3.83 (m, 1H), 2.89 (s, 3H), 2.75-2.86 (m, 1H), 2.56-2.68 (m, 1H), 1.57-1.66 (m, 1H), 1.46-1.56 (m, 1H), 1.30-1.45 (m, 1H), 0.75-0.99 (m, 9H). ¹H NMR (400 MHz, 100° C., DMSO-d₆) δ 9.22 (br, 1H), 5.70-5.90 (m, 1H), 5.12-5.60 (m, 1H), 4.90-5.11 (m, 1H), 4.60-4.88 (m, 1H), 3.71-4.10 (m, 2H), 2.86-2.98 (m, 3H), 2.70-2.85 (m, 1H), 2.60-2.69 (m, 1H), 1.30-1.80 (m, 3H), 0.50-1.15 (m, 9H). LC-MS (ESI, m/z): 576 [M+H]⁺.

### Example 27

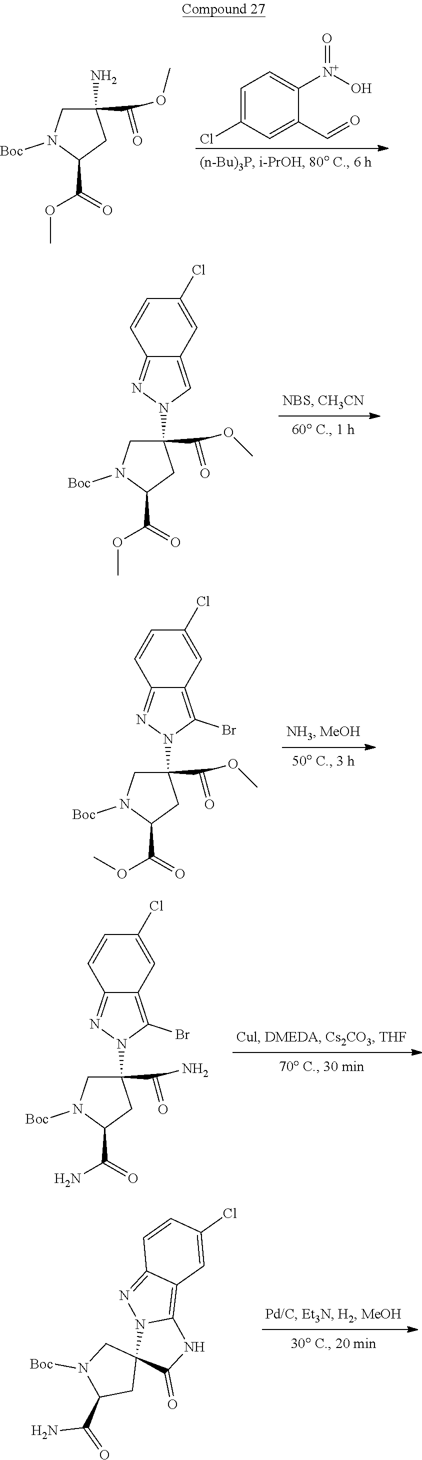

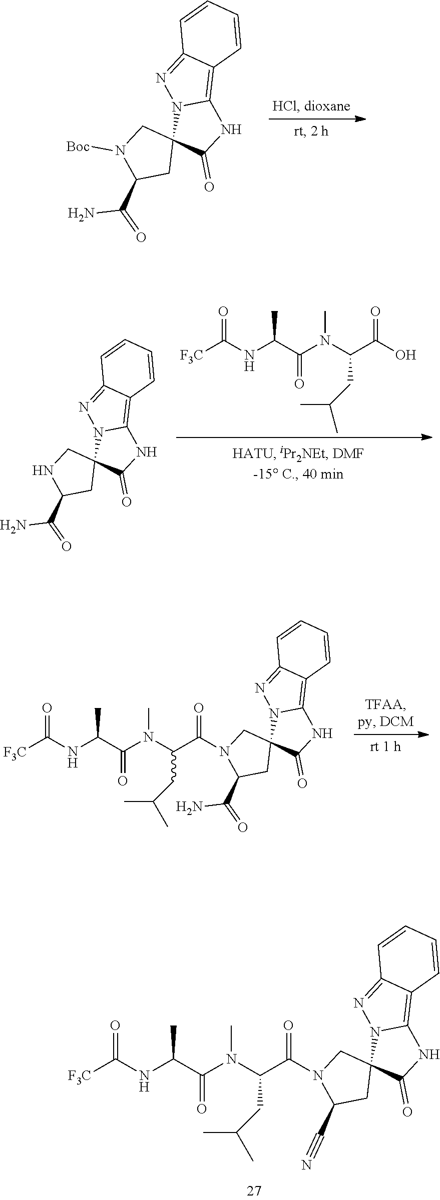 [SMILES: CC(C)(C)OC(=O)N1C[C@]2(C[C@H]1C(N)=O)C(=O)Nc1c3ccccc3nn12 ; NC(=O)[C@@H]1C[C@@]2(CN1)C(=O)Nc1c3ccccc3nn12 ; CC(C)C[C@@H](C(=O)O)N(C)C(=O)[C@H](C)NC(=O)C(F)(F)F ; CC(C)CC(C(=O)N1C[C@]2(C[C@H]1C(N)=O)C(=O)Nc1c3ccccc3nn12)N(C)C(=O)[C@H](C)NC(=O)C(F)(F)F ; CC(C)C[C@@H](C(=O)N1C[C@]2(C[C@H]1C#N)C(=O)Nc1c3ccccc3nn12)N(C)C(=O)[C@H](C)NC(=O)C(F)(F)F]

A mixture of 1-t-butyl 2,4-dimethyl (2S,4R)-4-aminopyrrolidine-1,2,4-tricarboxylate (800 mg, 2.64 mmol), 5-chloro-2-nitrobenzaldehyde (491 mg, 2.64 mmol) in isopropyl alcohol (7 mL) was stirred for 4 h at 80° C. under nitrogen, and then tributylphosphine (1.98 g, 9.79 mmol) was added. The mixture was stirred for 2 h at 80° C. under nitrogen and the reaction was quenched with water (15 mL). The mixture was extracted with EA (3×15 mL). The organic layers were combined, washed with brine (2×10 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to provide 1-t-butyl 2,4-dimethyl (2S,4R)-4-(5-chloroindazol-2-yl)pyrrolidine-1,2,4-tricarboxylate (800 mg, 69%) as a red oil.

To a mixture of 1-t-butyl 2,4-dimethyl (2S,4R)-4-(5-chloroindazol-2-yl)pyrrolidine-1,2,4-tricarboxylate (800 mg, 1.82 mmol) in MeCN (10 mL) was added N-bromosuccinimide (325 mg, 1.82 mmol) at 0° C. The mixture was stirred for 1 h at 60° C. The reaction was quenched with water (20 mL). The mixture was extracted with EA (3×20 mL). The organic layers were combined, washed with brine (2×20 mL) and dried over anhydrous sodium sulfate. The solids were removed by filtration and the filtrate was concentrated under reduced pressure. The crude product was chromatographed on a silica gel column with EA:PE (4:6) to provide 1-t-butyl 2,4-dimethyl (2S,4R)-4-(3-bromo-5-chloroindazol-2-yl)pyrrolidine-1,2,4-tricarboxylate (800 mg, 84%) as a red oil.

A mixture of 1-t-butyl 2,4-dimethyl (2S,4R)-4-(3-bromo-5-chloroindazol-2-yl)pyrrolidine-1,2,4-tricarboxylate (800 mg, 1.54 mmol) in ammonia (20 mL, 7 M in CH₃OH) was stirred for 3 days at 50° C. The mixture was concentrated under reduced pressure to afford t-butyl (2S,4R)-4-(3-bromo-5-chloroindazol-2-yl)-2,4-dicarbamoylpyrrolidine-1-carboxylate (800 mg, crude) as a yellow solid. LC-MS (ESI, m/z):486 [M+H]⁺.

To a mixture of t-butyl (2S,4R)-4-(3-bromo-5-chloroindazol-2-yl)-2,4-dicarbamoylpyrrolidine-1-carboxylate (800 mg, 1.64 mmol), cuprous iodide (94.0 mg, 0.493 mmol, 0.3 eq.) and cesium carbonate (1.07 g, 3.28 mmol) in THF (10 mL) was added N,N′-dimethyl-1,2-ethanediamine (144 mg, 1.64 mmol). The mixture was stirred for 30 min at 70° C. under nitrogen and the reaction was quenched with water (20 mL). The mixture was extracted with EA (3×20 mL). The organic layers were combined, washed with brine (2×20 mL) and dried over anhydrous sodium sulfate. The solids were removed by filtration and the filtrate was concentrated under reduced pressure. The crude product was chromatographed on a silica gel column with CH₃OH:DCM (3:97) to provide t-butyl (3R,5'S)-5′-carbamoyl-8-chloro-2-oxo-1H-spiro[imidazo[1,2-b]indazole-3,3′-pyrrolidine]-1′-carboxylate (400 mg, 59%) as a yellow solid.

To a mixture of t-butyl (3R,5'S)-5′-carbamoyl-8-chloro-2-oxo-1H-spiro[imidazo[1,2-b]indazole-3,3′-pyrrolidine]-1′-carboxylate (260 mg, 0.641 mmol) in CH₃OH (5 mL) were added triethylamine (52.0 mg, 0.513 mmol) and 10% palladium on activated carbon (65 mg). The mixture was stirred for 20 min at 30° C. under hydrogen. The mixture was filtered through a celite pad and washed with CH₃OH. The filtrate was concentrated under reduced pressure. The crude product was purified by prep-HPLC (Column: Xselect CSH Prep Fluoro-Phenyl OBD C18 Column, 30×150 mm, 5 m; Mobile Phase A: Water (0.1% FA), Mobile Phase B: ACN; Flow rate: 60 mL/min; Gradient: 19% B to 34% B in 8 min; Wave Length: 220 nm) to provide t-butyl (3R,5'S)-5′-carbamoyl-2-oxo-1H-spiro[imidazo[1,2-b]indazole-3,3′-pyrrolidine]-1′-carboxylate (140 mg, 58%) as a white solid.

¹H NMR (400 MHz, DMSO-d₆) δ 12.10 (br, 1H), 7.58-7.79 (m, 1H), 7.40-7.57 (m, 2H), 6.91-7.30 (m, 3H), 4.62-4.73 (m, 1H), 3.84-4.00 (m, 1H), 3.63-3.83 (m, 1H), 2.54-2.59 (m, 1H), 2.32-2.43 (m, 1H), 1.32-1.49 (m, 9H). LC-MS (ESI, m/z): 372 [M+H]⁺.

A mixture of t-butyl (3R,5'S)-5′-carbamoyl-2-oxo-1H-spiro[imidazo[1,2-b]indazole-3,3′-pyrrolidine]-1′-carboxylate (140 mg, 0.377 mmol) in HCl (3 mL, 4 M in dioxane) was stirred for 2 h at rt. The mixture was concentrated under reduced pressure to afford (3R,5'S)-2-oxo-1H-spiro[imidazo[1,2-b]indazole-3,3′-pyrrolidine]-5′-carboxamide (102 mg, crude) as a white solid.

To a mixture of (3R,5'S)-2-oxo-1H-spiro[imidazo[1,2-b]indazole-3,3′-pyrrolidine]-5′-carboxamide (102 mg, 0.377 mmol, 1.0 eq.), (2S)-4-methyl-2-[(2S)—N-methyl-2-(2,2,2-trifluoroacetamido)propanamido]pentanoic acid (236 mg, 0.754 mmol) and HATU (315 mg, 0.830 mmol) in DMF (3 mL) was added N-ethyl-N-isopropylpropan-2-amine (170 mg, 1.32 mmol) at −15° C. The mixture was stirred for 40 min at −15° C. The reaction was quenched with water (10 mL). The mixture was then extracted with EA (3×10 mL). The organic layers were combined, washed with brine (2×10 mL) and dried over anhydrous sodium sulfate. The solids were removed by filtration and the filtrate was concentrated under reduced pressure. The crude product was chromatographed on a silica gel column with MeOH:DCM (1:15) to provide (3R,5'S)-1′-(N-methyl-N-((2,2,2-trifluoroacetyl)-L-alanyl)leucyl)-2-oxo-1,2-dihydrospiro[imidazo[1,2-b]indazole-3,3′-pyrrolidine]-5′-carboxamide (120 mg, 55%) as an off-white solid.

To a mixture (3R,5'S)-1′-(N-methyl-N-((2,2,2-trifluoroacetyl)-L-alanyl)leucyl)-2-oxo-1,2-dihydrospiro[imidazo[1,2-b]indazole-3,3′-pyrrolidine]-5′-carboxamide (120 mg, 0.212 mmol) in DCM (3 mL) were added pyridine (84.0 mg, 1.06 mmol,) and trifluoroacetic anhydride (89.0 mg, 0.424 mmol). The mixture was stirred for 1 h at rt and the reaction was quenched with water (8 mL). The mixture was extracted with DCM (3×10 mL). The organic layers were combined, washed with brine (2×10 mL) and dried over anhydrous sodium sulfate. The solids were removed by filtration and the filtrate was concentrated under reduced pressure. The crude product was purified by prep-HPLC (Column: Xselect CSH Prep Fluoro-Phenyl OBD C18 Column, 30×150 mm, 5 m; Mobile Phase A: Water (0.1% FA), Mobile Phase B: ACN; Flow rate: 60 mL/min; Gradient: 37% B to 38% B in 10 min; Wave Length: 220 nm) to provide (2S)—N-[(2S)-1-[(3R,5'S)-5′-cyano-2-oxo-1H-spiro[imidazo[1,2-b]indazole-3,3′-pyrrolidin]-1′-yl]-4-methyl-1-oxopentan-2-yl]-N-methyl-2-(2,2,2-trifluoroacetamido)propanamide (22.3 mg, 19%) as a white solid. ¹H NMR (400 MHz, DMSO-d₆) δ 11.25-12.30 (m, 1H), 9.38-9.49 (m, 1H), 7.35-7.52 (m, 2H), 7.15-7.26 (m, 1H), 6.87-6.99 (m, 1H), 5.11-5.28 (m, 2H), 4.34-4.46 (m, 1H), 4.06-4.17 (m, 1H), 3.86-3.99 (m, 1H), 2.84-3.00 (m, 4H), 2.69-2.81 (m, 1H), 1.58-1.68 (m, 1H), 1.47-1.57 (m, 1H), 1.31-1.45 (m, 1H), 0.75-1.01 (m, 6H), 0.37-0.50 (m, 3H). ¹H NMR (400 MHz, 100° C., DMSO-d₆) δ 8.90-9.20 (m, 1H), 7.45-7.53 (m, 1H), 7.39-7.44 (m, 1H), 7.11-7.21 (m, 1H), 6.90-6.96 (m, 1H), 5.10-5.40 (m, 2H), 4.33-4.62 (m, 1H), 3.82-4.10 (m, 2H), 2.90-3.00 (m, 3H), 2.70-2.81 (m, 2H), 1.68-1.80 (m, 1H), 1.32-1.67 (m, 2H), 0.82-0.96 (m, 6H), 0.35-0.81 (m, 3H). LC-MS (ESI, m/z): 548 [M+H]⁺.

### Example 28

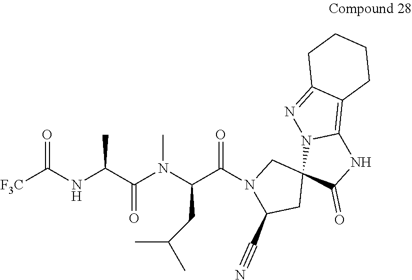 [SMILES: CC(C)C[C@H](C(=O)N1C[C@]2(C[C@H]1C#N)C(=O)Nc1c3c(nn12)CCCC3)N(C)C(=O)[C@H](C)NC(=O)C(F)(F)F]

To a mixture of t-butyl (3R,5'S)-5′-carbamoyl-8-chloro-2-oxo-1H-spiro[imidazo[1,2-b]indazole-3,3′-pyrrolidine]-1′-carboxylate (150 mg, 0.370 mmol, 1.0 eq.) in EtOH (3 mL) was added 10% palladium on activated carbon (75 mg). The mixture was stirred for 40 mins at 50° C. under hydrogen. The mixture was filtered through a celite pad and washed with EtOH. The filtrate was concentrated under reduced pressure to provide t-butyl (3R,5'S)-5′-carbamoyl-2-oxo-6,7,8,9-tetrahydro-1H-spiro[imidazo[1,2-b]indazole-3,3′-pyrrolidine]-1′-carboxylate (70 mg, 50%) as an off-white solid. ¹H NMR (400 MHz, DMSO-d₆) δ 11.40 (br, 1H), 7.53-7.65 (m, 1H), 6.99-7.14 (m, 1H), 4.44-4.53 (m, 1H), 3.57-3.69 (m, 2H), 2.51-2.56 (m, 2H), 2.21-2.41 (m, 4H), 1.60-1.79 (m, 4H), 1.33-1.43 (m, 9H). LC-MS (ESI, m/z): 376 [M+H]⁺.

A mixture of t-butyl (3R,5'S)-5′-carbamoyl-2-oxo-6,7,8,9-tetrahydro-1H-spiro[imidazo[1,2-b]indazole-3,3′-pyrrolidine]-1′-carboxylate (70.0 mg, 0.186 mmol, 1.0 eq.) in hydrogen chloride (1.5 mL, 4 M in dioxane) was stirred for 2 h at rt. The mixture was concentrated under reduced pressure to afford (3R,5'S)-2-oxo-6,7,8,9-tetrahydro-1H-spiro[imidazo[1,2-b]indazole-3,3′-pyrrolidine]-5′-carboxamide (51 mg, crude) as a white solid. LC-MS (ESI, m/z): 276 [M+H]⁺.

To a mixture of (3R,5'S)-2-oxo-6,7,8,9-tetrahydro-1H-spiro[imidazo[1,2-b]indazole-3,3′-pyrrolidine]-5′-carboxamide (51.0 mg, 0.185 mmol), (2S)-4-methyl-2-[(2S)—N-methyl-2-(2,2,2-trifluoroacetamido)propanamido]pentanoic acid (58.0 mg, 0.185 mmol) and HATU (120 mg, 0.314 mmol) in DMF (1 mL) was added N-ethyl-N-isopropylpropan-2-amine (60.0 mg, 0.463 mmol) at −15° C. The mixture was stirred for 40 min at −15° C. The reaction was quenched with water (5 mL). The mixture was extracted with EA (3×5 mL). The organic layers were combined, washed with brine (2×5 mL) and dried over anhydrous sodium sulfate. The solids were removed by filtration and the filtrate was concentrated under reduced pressure. The crude product was chromatographed on a silica gel column with CH₃OH:DCM (9:91) to provide (3R,5'S)-1′-(N-methyl-N-((2,2,2-trifluoroacetyl)-L-alanyl)leucyl)-2-oxo-1,2,6,7,8,9-hexahydrospiro[imidazo[1,2-b]indazole-3,3′-pyrrolidine]-5′-carboxamide (40.0 mg, 37%) as a white solid.

To a mixture (3R,5'S)-1′-(N-methyl-N-((2,2,2-trifluoroacetyl)-L-alanyl)leucyl)-2-oxo-1,2,6,7,8,9-hexahydrospiro[imidazo[1,2-b]indazole-3,3′-pyrrolidine]-5′-carboxamide (40.0 mg, 0.070 mmol) in DCM (1 mL) were added pyridine (36.0 mg, 0.455 mmol) and trifluoroacetic anhydride (38.0 mg, 0.182 mmol). The mixture was stirred for 1 h at rt. The reaction was quenched with water (3 mL). The mixture was extracted with DCM (3×3 mL). The organic layers were combined, washed with brine (2×3 mL) and dried over anhydrous sodium sulfate. The solids were removed by filtration and the filtrate was concentrated under reduced pressure. The crude product was purified by prep-HPLC (Column: Xselect CSH Prep Fluoro-Phenyl OBD C18 Column, 30×150 mm, 5 m; Mobile Phase A: Water (0.1% FA), Mobile Phase B: ACN; Flow rate: 60 mL/min; Gradient: 35% B to 44% B in 9 min; Wave Length: 220 nm) to provide (2S)—N-[(2S)-1-[(3R,5'S)-5′-cyano-2-oxo-6,7,8,9-tetrahydro-1H-spiro[imidazo[1,2-b]indazole-3,3′-pyrrolidin]-1′-yl]-4-methyl-1-oxopentan-2-yl]-N-methyl-2-(2,2,2-trifluoroacetamido)propanamide (5.1 mg, 13%) as a white solid. ¹H NMR (400 MHz, DMSO-d₆) δ 9.62 (br, 1H), 8.42 (br, 1H), 5.13-5.23 (m, 1H), 4.95-5.05 (m, 1H), 4.45-4.63 (m, 1H), 3.86-3.94 (m, 1H), 3.59-3.67 (m, 1H), 2.85 (s, 3H), 2.63-2.74 (m, 1H), 2.53-2.60 (m, 1H), 2.23-2.41 (m, 4H), 1.44-1.77 (m, 6H), 1.29-1.43 (m, 1H), 0.72-0.94 (m, 9H). ¹H NMR (400 MHz, 100° C., DMSO-d₆) δ 8.00-9.70 (m, 1H), 5.10-5.30 (m, 1H), 4.90-5.09 (m, 1H), 4.50-4.66 (m, 1H), 3.60-3.88 (m, 2H), 2.82-2.92 (m, 4H), 2.60-2.72 (m, 1H), 2.49-2.59 (m, 1H), 2.20-2.40 (m, 3H), 1.60-1.80 (m, 5H), 1.30-1.59 (m, 2H), 0.80-1.00 (m, 9H). LC-MS (ESI, m/z): 552 [M+H]⁺.

### Example 29

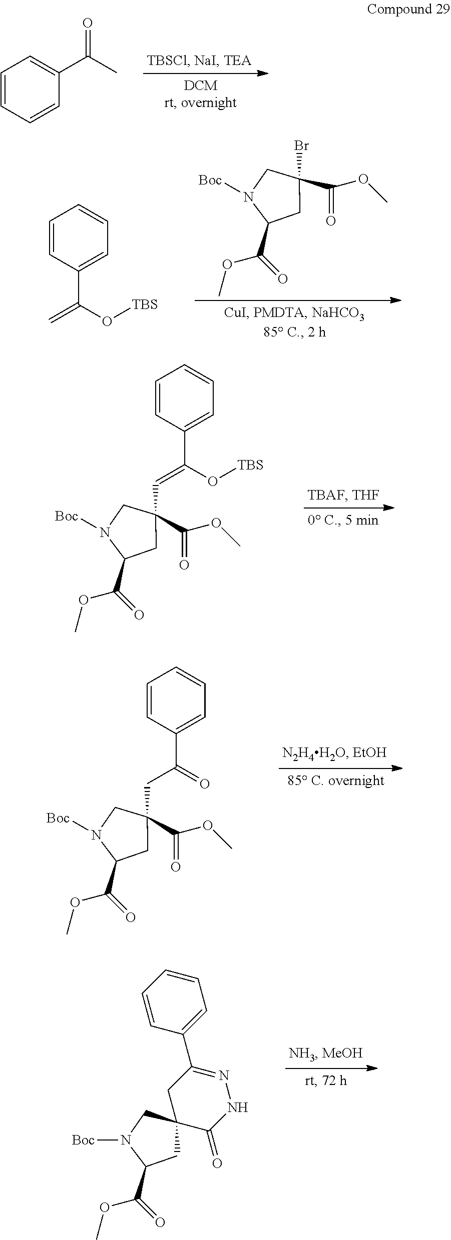

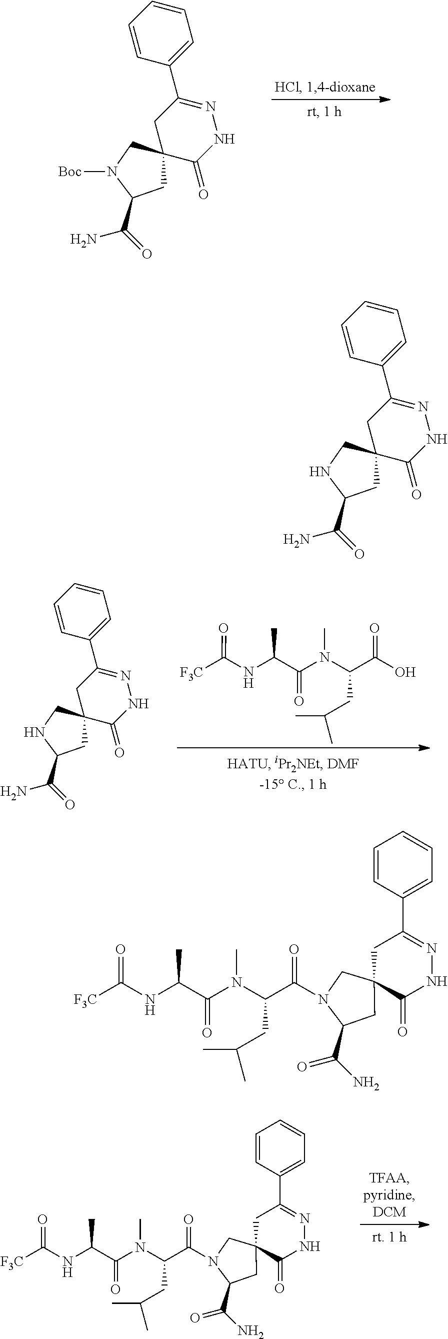 [SMILES: CC(C)(C)OC(=O)N1C[C@]2(CC(c3ccccc3)=NNC2=O)C[C@H]1C(N)=O ; NC(=O)[C@@H]1C[C@]2(CN1)CC(c1ccccc1)=NNC2=O ; NC(=O)[C@@H]1C[C@]2(CN1)CC(c1ccccc1)=NNC2=O ; CC(C)C[C@@H](C(=O)O)N(C)C(=O)[C@H](C)NC(=O)C(F)(F)F ; CC(C)C[C@@H](C(=O)N1C[C@]2(CC(c3ccccc3)=NNC2=O)C[C@H]1C(N)=O)N(C)C(=O)[C@H](C)NC(=O)C(F)(F)F ; CC(C)C[C@@H](C(=O)N1C[C@]2(CC(c3ccccc3)=NNC2=O)C[C@H]1C(N)=O)N(C)C(=O)[C@H](C)NC(=O)C(F)(F)F]

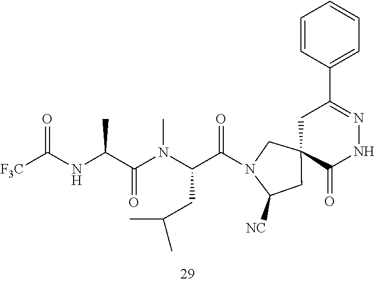

To a solution of acetophenone (6.00 g, 49.9 mmol, 1.0 eq.), t-butyldimethylchlorosilane (11.3 g, 74.9 mmol, 1.5 eq.) and sodium iodide (15.0 g, 99.9 mmol, 2.0 eq.) in ACN (60 mL) was added trimethylamine (7.58 g, 74.9 mmol, 3.0 eq.). The mixture was stirred overnight at rt and the reaction was quenched with water (200 mL). The mixture was extracted with EA (3×300 mL). The organic layers were combined, washed with brine (2×200 mL) and dried over anhydrous sodium sulfate. The solids were removed by filtration and the filtrate was concentrated under reduced pressure. The crude product was chromatographed on a silica gel column with EA:PE (3%-4%) to provide t-butyldimethyl((1-phenylvinyl)oxy)silane (6.6 g, 56%) as a colorless liquid. ¹H NMR (400 MHz, DMSO-d₆) δ 7.58-7.64 (m, 2H), 7.27-7.40 (m, 3H), 5.01 (s, 1H), 4.42 (s, 1H), 0.97 (s, 9H), 0.19 (s, 6H). GC-MS (ESI, m/z): 234 [M].

To a solution of 1-t-butyl 2,4-dimethyl (2S,4R)-4-bromopyrrolidine-1,2,4-tricarboxylate (1.00 g, 2.73 mmol, 1.0 eq.), t-butyldimethyl((1-phenylvinyl)oxy)silane (1.28 g, 5.46 mmol, 2.0 eq.), cuprous iodide (98.8 mg, 0.519 mmol, 0.19 eq.) and sodium bicarbonate (413 mg, 4.91 mmol, 1.80 eq.) in 1,4-dioxane (10 ml) was added N,N,N′,N″,N″-Pentamethyldiethylenetriamine (94.7 mg, 0.546 mmol, 0.20 eq.) under nitrogen. The mixture was stirred for 2 h at 85° C. and the reaction was quenched with water (50 mL). The mixture was extracted with EA (3×150 mL). The organic layers were combined, washed with brine (2×50 mL) and dried over anhydrous sodium sulfate. The solids were removed by filtration and the filtrate was concentrated under reduced pressure. The crude product was chromatographed on a silica gel column with EA:PE (18%-20%) to provide 1-(t-butyl) 2,4-dimethyl (2S,4R)-4-((Z)-2-((t-butyldimethylsilyl)oxy)-2-phenylvinyl)pyrrolidine-1,2,4-tricarboxylate (680 mg, crude) as a light yellow oil. LC-MS (ESI, m/z): 520 [M+H]⁺.

To a solution of 1-(t-butyl) 2,4-dimethyl (2S,4R)-4-((Z)-2-((t-butyldimethylsilyl)oxy)-2-phenylvinyl)pyrrolidine-1,2,4-tricarboxylate (640 mg, 1.23 mmol) in THF (4.5 mL) was added TBAF (0.62 mL, 0.62 mmol, 1M in THF) stirred at 0° C. The mixture was stirred for 5 mins at 0° C. and the reaction was quenched with water (50 mL). The mixture was extracted with EA (3×100 mL). The organic layers were combined, washed with brine (2×100 mL) and dried over anhydrous sodium sulfate. The solids were removed by filtration and the filtrate was concentrated under reduced pressure to afford 1-(t-butyl) 2,4-dimethyl (2S,4R)-4-(2-oxo-2-phenylethyl)pyrrolidine-1,2,4-tricarboxylate (450 mg, crude) as a yellow solid. LC-MS (ESI, m/z): 306 [M−Boc+H]⁺.

To a solution of 1-(t-butyl) 2,4-dimethyl (2S,4R)-4-(2-oxo-2-phenylethyl)pyrrolidine-1,2,4-tricarboxylate (1.00 g, 2.46 mmol) in EtOH (13 mL) was added hydrazine hydrate (426 mg, 8.50 mmol) under nitrogen at rt. The mixture was stirred overnight at 85° C. and the reaction was quenched with water (50 mL). The mixture was extracted with EA (3×100 mL) and dried over anhydrous sodium sulfate. The solids were removed by filtration and the filtrate was concentrated under reduced pressure. The crude product was chromatographed on a silica gel column with EA:PE (25%-30%) to provide 2-(t-butyl) 3-methyl (3S,5R)-6-oxo-9-phenyl-2,7,8-triazaspiro[4.5]dec-8-ene-2,3-dicarboxylate (380 mg, 38%) as a white solid. LC-MS (ESI, m/z): 288 [M−Boc+H]⁺.

A mixture of 2-(t-butyl) 3-methyl (3S,5R)-6-oxo-9-phenyl-2,7,8-triazaspiro[4.5]dec-8-ene-2,3-dicarboxylate (200 mg, 0.510 mmol) and ammonia (10 ml, 7M in CH₃OH) was stirred for 72 h at rt. The mixture was concentrated under reduced pressure. The crude product was purified by prep-HPLC (Column: XBridge Prep OBD C18 Column, 30×150 mm, 5 μm; Mobile Phase A: Water (0.05% TFA), Mobile Phase B: ACN; Flow rate: 60 mL/min; Gradient: 16% B to 46% B in 10 min; Wave Length: 254 nm/220 nm) to provide t-butyl (3S,5R)-3-acetyl-6-oxo-9-phenyl-2,7,8-triazaspiro[4.5]dec-8-ene-2-carboxylate (100 mg, 50%) as a white solid. ¹H NMR (400 MHz, DMSO-d₆) δ 11.21 (s, 1H), 7.69-7.81 (m, 2H), 7.39-7.46 (m, 3H), 6.91-7.37 (m, 2H), 4.11-4.27 (m, 1H), 3.35-3.56 (m, 2H), 2.83-3.06 (m, 2H), 2.06-2.26 (m, 2H), 1.21-1.42 (m, 9H). LC-MS (ESI, m/z): 373 [M+H]⁺.

To a stirred solution of t-butyl (3S,5R)-3-carbamoyl-6-oxo-9-phenyl-2,7,8-triazaspiro[4.5]dec-8-ene-2-carboxylate (90 mg, 0.242 mmol) in 1,4-dioxane (1 mL) was added hydrogen chloride (2 mL, 4M in 1,4-dioxane). The mixture was stirred for 1 h at rt and then concentrated under reduced pressure to afford (3S,5R)-6-oxo-9-phenyl-2,7,8-triazaspiro[4.5]dec-8-ene-3-carboxamide (60 mg, crude) as a yellow solid. LC-MS (ESI, m/z): 273 [M+H]⁺.

A solution of (3S,5R)-6-oxo-9-phenyl-2,7,8-triazaspiro[4.5]dec-8-ene-3-carboxamide (60 mg, 0.220 mmol), (2S)-4-methyl-2-[(2S)—N-methyl-2-(2,2,2-trifluoroacetamido)propanamido]pentanoic acid (68.8 mg, 0.220 mmol) and HATU (101 mg, 0.264 mmol) in DMF (3 mL) was stirred for 5 min at −15° C. N-ethyl-N-isopropylpropan-2-amine (42.7 mg, 0.33 mmol) was added slowly at −15° C. The mixture was stirred for 1 h at −15° C. and the reaction was quenched with water (20 mL). The mixture was extracted with EA (3×30 mL). The organic layers were combined, washed with brine (2×20 mL) and dried over anhydrous sodium sulfate. The solids were removed by filtration and the filtrate was concentrated under reduced pressure. The crude product was purified by TLC (Mobile phase: CH₃OH:DCM=1:11; detection: UV) to provide (3S,5R)-2-[(2S)-4-methyl-2-[(2S)—N-methyl-2-(2,2,2-trifluoroacetamido)propanamido]pentanoyl]-6-oxo-9-phenyl-2,7,8-triazaspiro[4.5]dec-8-ene-3-carboxamide (70 mg, 56%) as a yellow oil. LC-MS (ESI, m/z): 567 [M+H]⁺.

To a solution of (3S,5R)-2-(N-methyl-N-((2,2,2-trifluoroacetyl)-L-alanyl)-L-leucyl)-6-oxo-9-phenyl-2,7,8-triazaspiro[4.5]dec-8-ene-3-carboxamide (70 mg, 0.124 mmol) in DCM (0.7 ml) were added pyridine (58.6 mg, 0.744 mmol) and trifluoroacetic anhydride (51.9 mg, 0.248 mmol). The mixture was stirred for 1 h at rt and the reaction was quenched with water (20 mL). The mixture was extracted with DCM (3×30 mL). The organic layers were combined, washed with brine (2×20 mL) and dried over anhydrous sodium sulfate. The solids were removed by filtration and the filtrate was concentrated under reduced pressure. The crude product was purified by prep-HPLC Column: Xselect CSH Prep C18 Column, 30×150 mm, 5 m; Mobile Phase A: Water (0.1% FA), Mobile Phase B: ACN; Flow rate: 60 mL/min; Gradient: 33% B to 63% B in 10 min; Wave Length: 254 nm/220 nm) to provide (S)—N—((S)-1-((3S,5R)-3-cyano-6-oxo-9-phenyl-2,7,8-triazaspiro[4.5]dec-8-en-2-yl)-4-methyl-1-oxopentan-2-yl)-N-methyl-2-(2,2,2-trifluoroacetamido)propanamide (25.7 mg 37%) as white solid. ¹H NMR (400 MHz, 80° C., DMSO-d₆) δ 11.0-11.3 (m, 1H), 9.44 (br, 1H), 7.74 (br, 2H), 7.30-7.55 (m, 3H), 5.03-5.31 (m, 1H), 4.83-5.01 (m, 1H), 4.55-4.78 (m, 1H), 3.75-4.05 (m, 1H), 3.41-3.55 (m, 1H), 3.12-3.25 (m, 2H), 2.95-3.11 (m, 2H), 2.81-2.93 (m, 1H), 2.31-2.45 (m, 2H), 1.41-1.71 (m, 3H), 1.01-1.38 (m, 3H), 0.91-0.99 (m, 3H), 0.81-0.89 (m, 3H). LC-MS (ESI, m/z): 571 [M+Na]⁺.

### Example 30

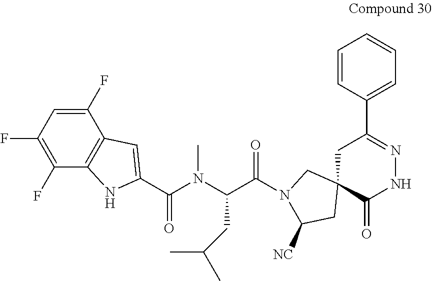 [SMILES: CC(C)C[C@@H](C(=O)N1C[C@]2(CC(c3ccccc3)=NNC2=O)C[C@H]1C#N)N(C)C(=O)c1cc2c(F)cc(F)c(F)c2[nH]1]

A solution of (3S,5R)-2-(methyl-L-leucyl)-6-oxo-9-phenyl-2,7,8-triazaspiro[4.5]dec-8-ene-3-carboxamide (90 mg, 0.220 mmol, 1.0 eq.), 4,6,7-trifluoro-1H-indole-2-carboxylic acid (68.8 mg, 0.220 mmol) and HATU (101 mg, 0.264 mmol) in DMF (5 mL) and DCM (1 mL) was added N-ethyl-N-isopropylpropan-2-amine (42.7 mg, 0.33 mmol) at 0° C. The mixture was stirred for 1 h at rt and the reaction was quenched with water (20 mL). The mixture was extracted with EA (3×50 mL). The organic layers were combined, washed with brine (3×50 mL) and dried over anhydrous sodium sulfate. The solids were removed by filtration and the filtrate was concentrated under reduced pressure. The crude product was purified by TLC (Mobile phase: CH₃OH:DCM=1:10; detection: UV) to provide (3S,5R)-2-(N-methyl-N-(4,6,7-trifluoro-1H-indole-2-carbonyl)-L-leucyl)-6-oxo-9-phenyl-2,7,8-triazaspiro[4.5]dec-8-ene-3-carboxamide (100 mg, 74%) as a white solid.

To a solution of (3S,5R)-2-(N-methyl-N-(4,6,7-trifluoro-1H-indole-2-carbonyl)-L-leucyl)-6-oxo-9-phenyl-2,7,8-triazaspiro[4.5]dec-8-ene-3-carboxamide (100 mg, 0.17 mmol) in DCM (2 ml) were added pyridine (66.3 mg, 0.84 mmol) and 2,2,2-trifluoroacetic anhydride (70.4 mg, 0.340 mmol). The mixture was stirred for 1 h at rt and the reaction was quenched with water (20 mL). The mixture was extracted with DCM (3×30 mL). The organic layers were combined, washed with brine (2×20 mL) and dried over anhydrous sodium sulfate. The solids were removed by filtration and the filtrate was concentrated under reduced pressure. The crude product was purified by prep-HPLC (Column: Xselect CSH Prep C18 Column, 30×150 mm, 5 m; Mobile Phase A: Water (0.1% FA), Mobile Phase B: ACN; Flow rate: 60 mL/min; Gradient: 47 to 77% B in 10 min; Wave Length: 254 nm/220 nm) to provide N—((S)-1-((3S,5R)-3-cyano-6-oxo-9-phenyl-2,7,8-triazaspiro[4.5]dec-8-en-2-yl)-4-methyl-1-oxopentan-2-yl)-4,6,7-trifluoro-N-methyl-1H-indole-2-carboxamide (44.7 mg, 45%) as white solid. ¹H NMR (400 MHz, 80° C., DMSO-d₆) δ 12.3 (s, 1H), 11.1 (s, 1H), 7.61-7.82 (m, 2H), 7.20-7.41 (m, 3H), 6.95-7.05 (m, 1H), 6.81-6.90 (m, 1H), 5.25-5.51 (m, 1H), 4.91-5.11 (m, 1H), 3.81-4.10 (m, 1H), 3.51-3.70 (m, 1H), 3.11-3.22 (m, 3H), 2.75-3.01 (m, 2H), 2.21-2.45 (m, 2H), 1.51-1.92 (m, 3H), 0.81-1.02 (m, 6H). LC-MS (ESI, m/z): 601 [M+Na]⁺.

### Example 31

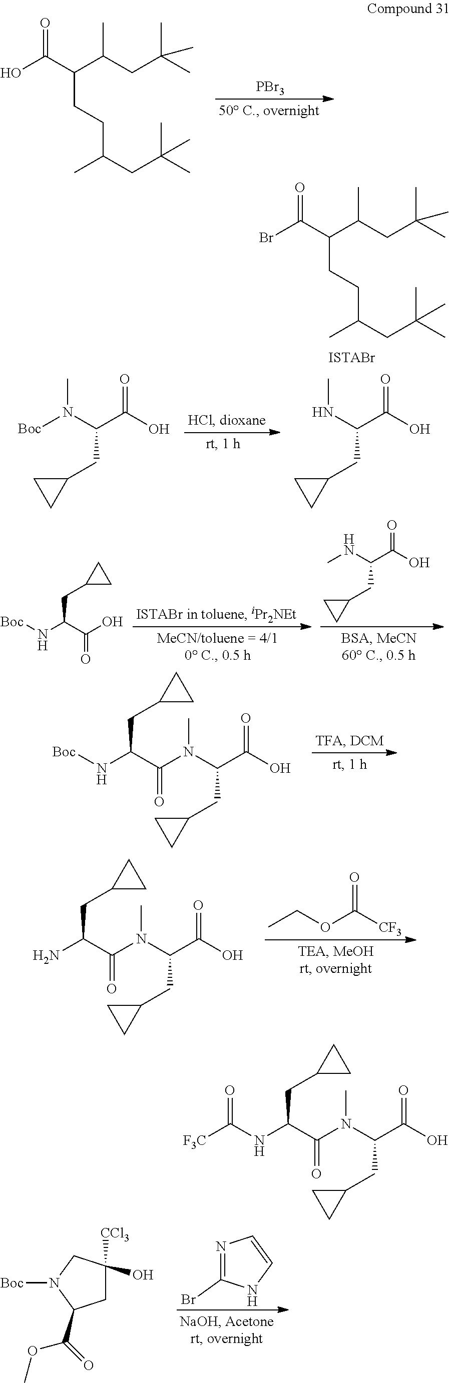

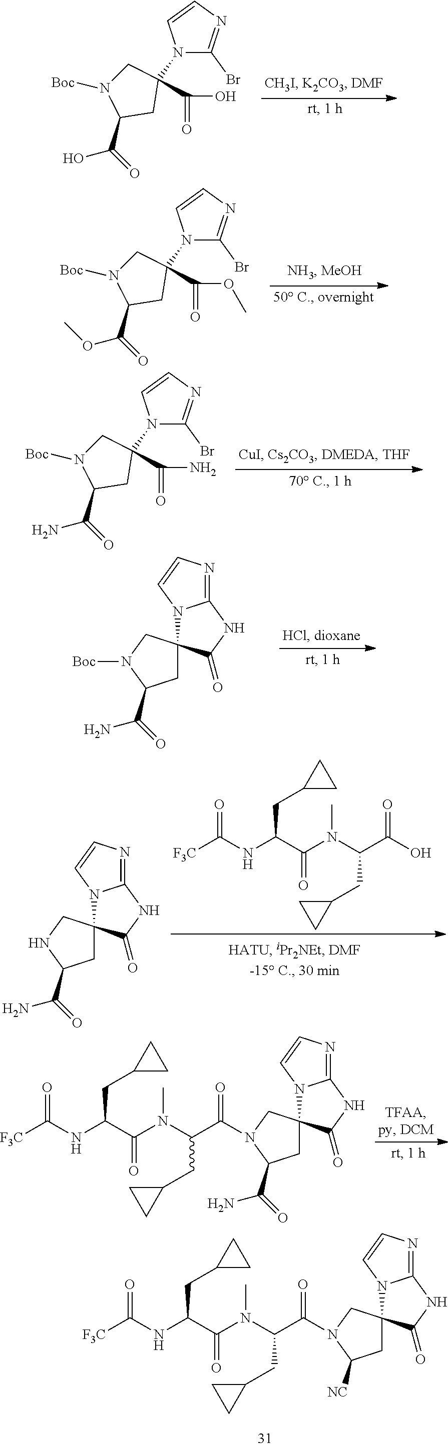

Phosphorus tribromide (5.71 g, 21.1 mmol) was added dropwise to 2-(4,4-dimethylpentan-2-yl)-5,7,7-trimethyloctanoic acid (5.0 g, 17.6 mmol) at 0° C. The reaction mixture was stirred overnight at 50° C. The mixture was concentrated under reduced pressure to afford 2-(4,4-dimethylpentan-2-yl)-5,7,7-trimethyloctanoyl bromide (2.5 g, ISTABr, crude) as a yellow oil.

A stirred mixture of (S)-2-((t-butoxycarbonyl)(methyl)amino)-3-cyclopropylpropanoic acid (1.0 g, 4.11 mmol) in HCl (10 mL, 4 M in dioxane) was stirred for 1 h at rt. The mixture was concentrated under reduced pressure to afford (S)-3-cyclopropyl-2-(methylamino)propanoic acid (588 mg, crude) as a light yellow solid. LC-MS (ESI, m/z): 144 [M+H]⁺.

To a mixture of (S)-2-((t-butoxycarbonyl)amino)-3-cyclopropylpropanoic acid (857 mg, 3.74 mmol) and 2-(4,4-dimethylpentan-2-yl)-5,7,7-trimethyloctanoyl bromide (2.73 g, 3.92 mmol, 50% pure) in CH₃CN (8 mL)/toluene (2 mL) was added N-ethyl-N-isopropylpropan-2-amine (773 mg, 5.98 mmol) at 0° C. The mixture (A) was stirred for 0.5 h at 0° C. At the same time, to a mixture of (S)-3-cyclopropyl-2-(methylamino)propanoic acid (588 mg, 4.10 mmol) in CH₃CN (6 mL) was added N,O-bis(trimethylsilyl)acetamide (2.00 g, 12.3 mmol). The mixture was stirred for 0.5 h at 60° C. and then added to mixture (A) at 0° C. The resulting mixture was stirred overnight at rt and the reaction was quenched with potassium bisulfate aqueous solution (30 mL, 1 M) at 0° C. The mixture was extracted with EA (3×30 mL). The organic layers were combined, washed with brine (2×30 mL) and dried over anhydrous sodium sulfate. The solids were removed by filtration and the filtrate was concentrated under reduced pressure. The crude product was purified by C18 column with CH₃CN/Water (0.05% FA). The fraction was concentrated under reduced pressure to provide (S)-2-((S)-2-((t-butoxycarbonyl)amino)-3-cyclopropyl-N-methylpropanamido)-3-cyclopropylpropanoic acid (700 mg, 53%) as a yellow solid. ¹H NMR (400 MHz, DMSO-d₆) δ 12.24-12.80 (m, 1H), 6.87-7.11 (m, 1H), 4.75-5.16 (m, 1H), 4.34-4.60 (m, 1H), 2.92-3.13 (m, 2H), 2.57-2.82 (m, 1H), 1.69-1.90 (m, 1H), 1.30-1.66 (m, 12H), 0.51-0.82 (m, 2H), −0.15-0.48 (m, 8H). LC-MS (ESI, m/z): 355 [M+H]⁺.

To a stirred mixture of (S)-2-((S)-2-((t-butoxycarbonyl)amino)-3-cyclopropyl-N-methylpropanamido)-3-cyclopropylpropanoic acid (700 mg, 1.98 mmol) in DCM (6 mL) was added TFA (2 mL). The mixture was stirred for 1 h at rt and then concentrated under reduced pressure to afford (S)-2-((S)-2-amino-3-cyclopropyl-N-methylpropanamido)-3-cyclopropylpropanoic acid (502 mg, crude) as a yellow oil. LC-MS (ESI, m/z): 255 [M+H]⁺.

To a mixture of (S)-2-((S)-2-amino-3-cyclopropyl-N-methylpropanamido)-3-cyclopropylpropanoic acid (502 mg, 1.98 mmol) in CH₃OH (5 mL) were added trimethylamine (2.40 g, 23.7 mmol) and ethyl 2,2,2-trifluoroacetate (2.80 g, 19.7 mmol). The mixture was stirred overnight at rt and concentrated under reduced pressure to remove the solvent. The residue was diluted with H₂O (10 mL) and adjusted to pH=5 with HCl (2 M). The mixture was extracted with EA (3×20 mL). The organic layers were combined, washed with brine (2×10 mL) and dried over anhydrous sodium sulfate. The solids were removed by filtration and the filtrate was concentrated under reduced pressure to afford (S)-3-cyclopropyl-2-((S)-3-cyclopropyl-N-methyl-2-(2,2,2-trifluoroacetamido)propanamido)propanoic acid (700 mg, crude) as a yellow oil. ¹H NMR (400 MHz, DMSO-d₆) δ 9.59-9.88 (m, 1H), 4.89-5.07 (m, 1H), 4.77-4.88 (m, 1H), 4.51-4.61 (m, 0.22H), 2.87-3.16 (m, 2H), 2.66-2.80 (m, 1H), 1.45-1.84 (m, 4H), 0.56-0.83 (m, 2H), −0.04-0.44 (m, 8H). LC-MS (ESI, m/z): 351 [M+H]⁺.

To a mixture of 1-t-butyl 2-methyl (2S,4S)-4-hydroxy-4-(trichloromethyl)pyrrolidine-1,2-dicarboxylate (10.0 g, 27.6 mmol) and 2-bromo-1H-imidazole (8.11 g, 55.1 mmol) in acetone (150 mL) was added NaOH (6.62 g, 165 mmol) at 0° C. The mixture was stirred overnight at r. The mixture was adjusted to pH=7 with hydrogen chloride (4 M in dioxane) and then concentrated under reduced pressure to afford (2S,4R)-4-(2-bromoimidazol-1-yl)-1-(t-butoxycarbonyl)pyrrolidine-2,4-dicarboxylic acid (11.2 g, crude) as a light brown oil. LC-MS (ESI, m/z): 404 [M+H]⁺.

To a mixture of (2S,4R)-4-(2-bromoimidazol-1-yl)-1-(t-butoxycarbonyl)pyrrolidine-2,4-dicarboxylic acid (11.2 g, 27.7 mmol) and potassium carbonate (15.4 g, 110 mmol) in DMF (150 mL) was added methyl iodide (8.65 g, 61.0 mmol). The mixture was stirred for 1 h at rt and the reaction was quenched with water (300 mL). The mixture was extracted with EA (3×300 mL). The organic layers were combined, washed with brine (2×300 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to provide 1-t-butyl 2,4-dimethyl (2S,4R)-4-(2-bromoimidazol-1-yl)pyrrolidine-1,2,4-tricarboxylate (2.9 g, 22%) as a light brown semi-solid. ¹H NMR (400 MHz, DMSO-d₆) δ 7.57-7.65 (m, 1H), 6.95-7.03 (m, 1H), 4.39-4.59 (m, 1H), 4.18-4.38 (m, 1H), 4.04-4.13 (m, 1H), 3.68-3.74 (m, 6H), 3.18-3.14 (m, 1H), 2.65-2.83 (m, 1H), 1.27-1.33 (m, 9H). LC-MS (ESI, m/z): 432 [M+H]⁺.

To a mixture of (2S,4R)-4-(2-bromoimidazol-1-yl)-1-(t-butoxycarbonyl)pyrrolidine-2,4-dicarboxylic acid (11.2 g, 27.7 mmol) and potassium carbonate (15.4 g, 110 mmol) in DMF (150 mL) was added methyl iodide (8.65 g, 61.0 mmol). The mixture was stirred for 1 h at rt and the reaction was quenched with water (300 mL). The mixture was extracted with EA (3×300 mL). The organic layers were combined, washed with brine (2×300 mL) and dried over anhydrous sodium sulfate. The solids were removed by filtration and the filtrate was concentrated under reduced pressure t. The crude product was purified by C18 column with CH₃CN/Water (0.05% TFA). The fraction was concentrated under reduced pressure to provide 1-t-butyl 2,4-dimethyl (2S,4R)-4-(2-bromoimidazol-1-yl)pyrrolidine-1,2,4-tricarboxylate (2.9 g, 22%) as a light brown semi-solid. ¹H NMR (400 MHz, DMSO-d₆) δ 7.57-7.65 (m, 1H), 6.95-7.03 (m, 1H), 4.39-4.59 (m, 1H), 4.18-4.38 (m, 1H), 4.04-4.13 (m, 1H), 3.68-3.74 (m, 6H), 3.18-3.14 (m, 1H), 2.65-2.83 (m, 1H), 1.27-1.33 (m, 9H). LC-MS (ESI, m/z): 432 [M+H]⁺.

To a mixture of t-butyl (2S,4R)-4-(2-bromoimidazol-1-yl)-2,4-dicarbamoylpyrrolidine-1-carboxylate (800 mg, 2.00 mmol), copper(I) iodide (152 mg, 0.796 mmol, 0.4 eq.) and cesium carbonate (1.30 g, 4.00 mmol) in THF (10 mL) was added N,N′-dimethyl-1,2-ethanediamine (210 mg, 2.39 mmol). The mixture was stirred for 1 h at 70° C. under nitrogen and the reaction was quenched with water (20 mL). The mixture was adjusted to pH=5 with HCl (1 M) and then extracted with EA (2×20 mL). The aqueous phase was purified by C18 column with CH₃CN/Water (0.05% TFA). The fraction was concentrated under reduced pressure to provide t-butyl (3R,5'S)-5′-carbamoyl-2-oxo-1H-spiro[[1,3]diazolo[1,2-a]imidazole-3,3′-pyrrolidine]-1′-carboxylate (550 mg, 75%) as a light yellow solid. ¹H NMR (400 MHz, DMSO-d₆) δ 7.48-7.61 (m, 1H), 6.89-7.29 (m, 3H), 4.38-4.51 (m, 1H), 3.74-3.87 (m, 1H), 3.54-3.67 (m, 1H), 2.51-2.58 (m, 1H), 2.22-2.35 (m, 1H), 1.32-1.45 (m, 9H). LC-MS (ESI, m/z): 322 [M+H]⁺.

A mixture of t-butyl (3R,5'S)-5′-carbamoyl-2-oxo-1H-spiro[[1,3]diazolo[1,2-a]imidazole-3,3′-pyrrolidine]-1′-carboxylate (120 mg, 0.373 mmol) in HCl (3 mL, 4 M in dioxane) was stirred for 1 h at rt. The mixture was concentrated under reduced pressure to afford (3R,5'S)-2-oxo-1H-spiro[[1,3]diazolo[1,2-a]imidazole-3,3′-pyrrolidine]-5′-carboxamide (83.0 mg, crude) as a light yellow solid. LC-MS (ESI, m/z): 222 [M+H]⁺.

To a mixture of (3R,5'S)-2-oxo-1H-spiro[[1,3]diazolo[1,2-a]imidazole-3,3′-pyrrolidine]-5′-carboxamide (83.0 mg, 0.375 mmol), (2S)-3-cyclopropyl-2-[(2S)-3-cyclopropyl-N-methyl-2-(2,2,2-trifluoroacetamido)propanamido]propanoic acid (131 mg, 0.375 mmol) and HATU (171 mg, 0.450 mmol) in DMF (2 mL) was added N-ethyl-N-isopropylpropan-2-amine (73.0 mg, 0.563 mmol) at −15° C. The mixture was stirred for 30 min at −15° C. The reaction was quenched with water (5 mL) and then extracted with EA (3×5 mL). The organic layers were combined, washed with brine (2×5 mL) and dried over anhydrous sodium sulfate. The solids were removed by filtration and the filtrate was concentrated under reduced pressure. The crude product was chromatographed on a silica gel column with CH₃OH:DCM (9:91) to provide (3R,5'S)-1′-(3-cyclopropyl-2-((S)-3-cyclopropyl-N-methyl-2-(2,2,2-trifluoroacetamido)propanamido)propanoyl)-2-oxo-1,2-dihydrospiro[imidazo[1,2-a]imidazole-3,3′-pyrrolidine]-5′-carboxamide (65.0 mg, 30%) as an off-white solid.

To a mixture of (3R,5'S)-1′-(3-cyclopropyl-2-((S)-3-cyclopropyl-N-methyl-2-(2,2,2-trifluoroacetamido)propanamido)propanoyl)-2-oxo-1,2-dihydrospiro[imidazo[1,2-a]imidazole-3,3′-pyrrolidine]-5′-carboxamide (65.0 mg, 0.117 mmol, 1.0 eq.) in DCM (1.5 mL) were added pyridine (47.0 mg, 0.585 mmol) and trifluoroacetic anhydride (49.0 mg, 0.234 mmol). The mixture was stirred for 1 h at rt and the reaction was quenched with water (5 mL). The mixture was extracted with DCM (3×5 mL). The organic layers were combined, washed with brine (2×5 mL) and dried over anhydrous sodium sulfate. The solids were removed by filtration and the filtrate was concentrated under reduced pressure. The crude product was purified by prep-HPLC (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 30% B to 60% B in 8 min; Wave Length: 220 nm) to provide (2S)—N-[(2S)-1-[(3R,5'S)-5′-cyano-2-oxo-1H-spiro[[1,3]diazolo[1,2-a]imidazole-3,3′-pyrrolidin]-1′-yl]-3-cyclopropyl-1-oxopropan-2-yl]-3-cyclopropyl-N-methyl-2-(2,2,2-trifluoroacetamido)propanamide (7.1 mg, 11%) as a white solid. ¹H NMR (400 MHz, 80° C., DMSO-d₆) δ 9.30 (br, 1H), 6.72-6.82 (m, 1H), 6.60-6.70 (m, 1H), 5.00-5.22 (m, 2H), 4.50-4.75 (m, 1H), 3.65-4.00 (m, 2H), 2.92 (s, 3H), 2.60-2.75 (m, 1H), 2.50-2.59 (m, 1H), 1.50-1.65 (m, 2H), 1.10-1.49 (m, 2H), 0.40-0.65 (m, 2H), 0.20-0.39 (m, 4H), 0.06-0.15 (m, 1H), 0.00-0.05 (m, 2H), −0.15-−0.01 (m, 1H). LC-MS (ESI, m/z): 536 [M+H]⁺.

### Example 32

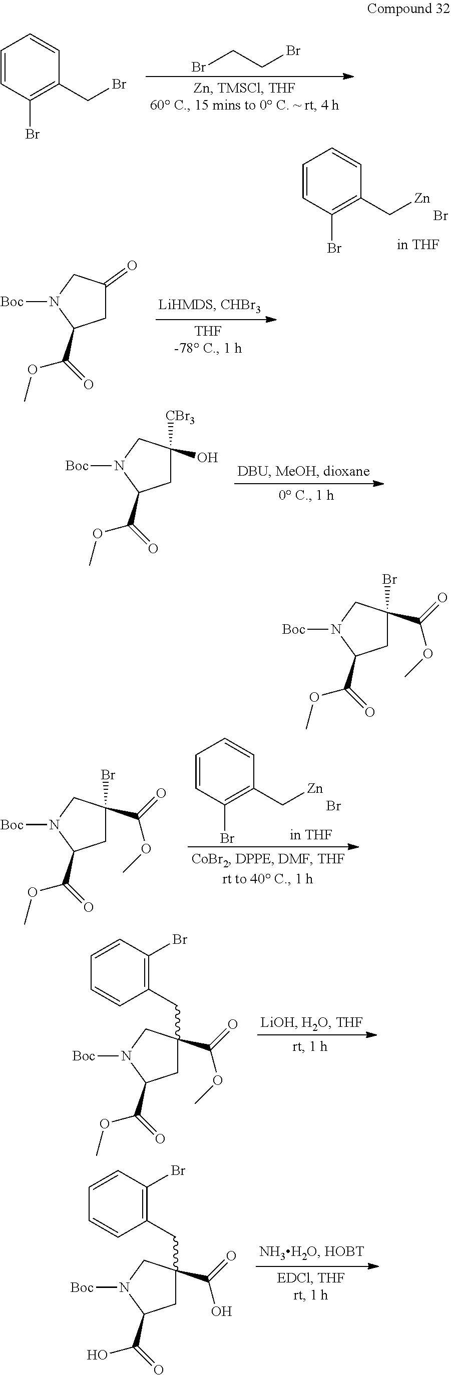

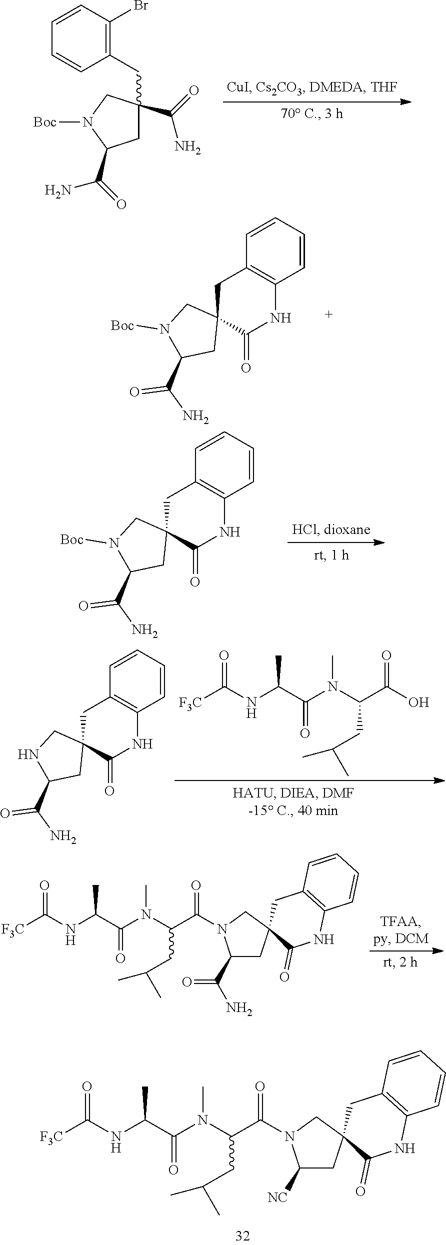

To a stirred suspension of zinc dust (523 mg, 8.00 mmol) in THF (8 mL) was added 1,2-dibromoethane (37.6 mg, 0.20 mmol) dropwise at rt. The mixture was placed in an oil bath, heated to reflux and stirred for 10 min. Chlorotrimethylsilane (8.69 mg, 0.08 mmol) was added dropwise. The mixture was stirred at 60° C. for 15 mins. 1-bromo-2-(bromomethyl)benzene (1.00 g, 4.00 mmol) in THF (2.0 mL) was added slowly at 0° C. The mixture was stirred at rt for 4 h (monitored by TLC). The stirring was discontinued, and the unreacted zinc was allowed to settle. The mixture was cooled to rt to afford a solution of (2-bromobenzyl)zinc(II) bromide (0.4 mmol/mL in THF).

To a solution of 1-(t-butyl) 2-methyl (S)-4-oxopyrrolidine-1,2-dicarboxylate (1.00 g, 4.11 mmol) and tribromomethane (2.08 g, 8.22 mmol) in THF (10 mL) was added dropwise lithium bis(trimethylsilyl)amide (8.2 mL, 8.22 mmol, 1 M in THF) at −78° C. under nitrogen. The mixture was stirred for 1 h at −78° C. and the reaction was quenched with NH₄Cl (sat., aq., 100 mL). The mixture was extracted with EA (3×150 mL). The organic layers were combined, washed with brine (2×80 mL) and dried over anhydrous sodium sulfate. The solids were removed by filtration and the filtrate was concentrated under reduced pressure. The crude product was chromatographed on a silica gel column with EA:PE (35%-45%) to provide 1-t-butyl 2-methyl (2S,4S)-4-hydroxy-4-(tribromomethyl)pyrrolidine-1,2-dicarboxylate (1.14 g, crude) as a light yellow solid. ¹H NMR (400 MHz, DMSO-d₆) δ 6.77-6.83 (m, 1H), 4.54-4.70 (m, 1H), 3.78-3.95 (m, 1H), 3.61-3.74 (m, 4H), 2.89-3.15 (m, 1H), 2.20-2.33 (m, 1H), 1.29-1.47 (m, 9H). LC-MS (ESI, m/z): 394 [M−100+H]⁺.

To a mixture of 1-(t-butyl) 2-methyl (2S,4S)-4-hydroxy-4-(tribromomethyl)pyrrolidine-1,2-dicarboxylate (500 mg, 1.01 mmol) and CH₃OH (113 mg, 3.55 mmol) in dioxane (5 mL) was added 1,8-diazabicyclo[5.4.0]undec-7-ene (339 mg, 2.23 mmol) at 0° C. The mixture was stirred for 1 h at 0° C. and the reaction was quenched with saturated ammonium chloride aqueous solution (20 mL). The mixture was extracted with EA (3×20 mL). The organic layers were combined, washed with brine (2×20 mL) and dried over anhydrous sodium sulfate. The solids were removed by filtration and the filtrate was concentrated under reduced pressure. The crude product was chromatographed on a silica gel column with EA:PE (1:4) to provide 1-(t-butyl) 2,4-dimethyl (2S,4R)-4-bromopyrrolidine-1,2,4-tricarboxylate (170 mg, >90% pure, −45% yield) as a light yellow oil. ¹H NMR (400 MHz, DMSO-d₆) δ 4.38-4.68 (m, 1H), 3.98-4.30 (m, 1H), 3.78-3.92 (m, 1H), 3.63-3.77 (m, 6H), 3.04-3.16 (m, 0.4H), 2.56-2.83 (m, 1.6H), 1.35-1.43 (m, 9H). LC-MS (ESI, m/z): 266 [M−Boc+H]⁺.

A solution of CoBr₂ (180 mg, 0.819 mmol) and 1,2-bis(diphenylphosphino)ethane (653 mg, 1.64 mmol) in DMF (30 mL) and THF (30 mL) was stirred for 15 mins at rt. 1-(t-butyl) 2,4-dimethyl (2S,4R)-4-bromopyrrolidine-1,2,4-tricarboxylate (3.00 g, 8.19 mmol) and the solution of (2-bromobenzyl)zinc(II) bromide (60.0 mL, 32.8 mmol) were added. The mixture was stirred for 1 h at 40° C. and the reaction was quenched with ammonium chloride solution (50 mL). The mixture was extracted with EA (3×200 mL). The organic layers were combined and dried over magnesium sulfate anhydrous. The solids were removed by filtration and the filtrate was concentrated under reduced pressure. The crude product was purified by C18 column with CH₃CN/Water (0.05% TFA), (68%). The fraction was concentrated under reduced pressure to provide 1-(t-butyl) 2,4-dimethyl (S)-4-(2-bromobenzyl)pyrrolidine-1,2,4-tricarboxylate (1.32 g, 35%) as a yellow oil. ¹H NMR (400 MHz, DMSO-d₆) δ 7.52-7.90 (m, 1H), 7.25-7.50 (m, 1H), 7.05-7.23 (m, 2H), 4.20-4.35 (m, 1H), 3.85-4.15 (m, 1H), 3.70-3.80 (m, 1H), 3.10-3.65 (m, 6H), 2.65-3.20 (m, 2H), 1.85-2.45 (m, 2H), 1.20-1.45 (m, 9H). LC-MS (ESI, m/z): 456 [M+H]⁺.

To a stirred mixture of 1-(t-butyl) 2,4-dimethyl (S)-4-(2-bromobenzyl)pyrrolidine-1,2,4-tricarboxylate (1.00 g, 1.97 mmol) in tetrahydrofuran (10 mL) and water (10 mL) was added lithium hydroxide (262 mg, 9.86 mmol) at rt. The mixture was stirred for 3 h and acidified to pH=3 with HCl (2M). The mixture was extracted with EA (3×150 mL). The organic layers were combined and dried over anhydrous sodium sulfate. The organic layers was concentrated under reduced pressure to afford (S)-4-(2-bromobenzyl)-1-(t-butoxycarbonyl)pyrrolidine-2,4-dicarboxylic acid (1.00 g, crude) as a yellow oil. LC-MS (ESI, m/z): 428 [M+H]⁺.

To a mixture of (S)-4-(2-bromobenzyl)-1-(t-butoxycarbonyl)pyrrolidine-2,4-dicarboxylic acid (900 mg, 2.10 mmol) in THF (9 mL) were added 1-hydroxybenzotriazole (1.71 g, 12.6 mmol) and 1-(3-dimethylaminopropyl)-3-ethylcarbodiimide HCl (2.00 g, 10.5 mmol). The mixture was stirred for 30 mins at 0° C. and then ammonia (15 mL) was added at 0° C. The mixture was stirred for 2 h at rt and the reaction was quenched with water (30 mL). The mixture was extracted with EA (3×150 mL). The organic layers were combined, washed with brine (2×30 mL) and dried over magnesium sulfate anhydrous. The solids were removed by filtration and the filtrate was concentrated under reduced pressure to afford t-butyl (S)-4-(2-bromobenzyl)-2,4-dicarbamoylpyrrolidine-1-carboxylate (870 mg, crude) as a yellow solid. LC-MS (ESI, m/z): 426 [M+H]⁺.

To a mixture of t-butyl (S)-4-(2-bromobenzyl)-2,4-dicarbamoylpyrrolidine-1-carboxylate (770 mg, 1.81 mmol) in THF (8 mL) were added cuprous iodide (206 mg, 1.08 mmol), cesium carbonate (1.18 g, 3.61 mmol) and dimethylethylenediamine (303 mg, 3.43 mmol). The mixture was stirred for 3 h at 70° C. under nitrogen and the reaction was quenched with water (25 mL). The mixture was extracted with EA (3×150 mL). The organic layers were combined, washed with brine (2×30 mL) and dried over magnesium sulfate anhydrous. The solids were removed by filtration and the filtrate was concentrated under reduced pressure. The crude product was chromatographed on a silica gel column with CH₃OH:DCM (7:93) to provide t-butyl (3S,5S)-5-carbamoyl-2′-oxo-1′,4′-dihydro-2′H-spiro[pyrrolidine-3,3′-quinoline]-1-carboxylate (260 mg, 41%, isomer ratio: 1/2.3) as a yellow solid. The two isomers were separated by prep-HPLC (Column: Xselect CSH Prep C18 OBD Column, 30×150 mm, 5 μm; Mobile Phase A: Water (0.1% FA), Mobile Phase B: ACN; Flow rate: 60 mL/min; Gradient: 26% B to 41% B in 8 min, 65% B; Wave Length: 220 nm) to provide t-butyl (3R,5S)-5-carbamoyl-2′-oxo-1′,4′-dihydro-2′H-spiro[pyrrolidine-3,3′-quinoline]-1-carboxylate (48.0 mg) and t-butyl (3S,5S)-5-carbamoyl-2′-oxo-1′,4′-dihydro-2′H-spiro[pyrrolidine-3,3′-quinoline]-1-carboxylate (116.0 mg) as white solids.

t-butyl (3R,5S)-5-carbamoyl-2′-oxo-1′,4′-dihydro-2′H-spiro[pyrrolidine-3,3′-quinoline]-1-carboxylate: ¹H NMR (400 MHz, DMSO-d₆) δ 10.24 (s, 1H), 7.35-7.58 (m, 1H), 7.10-7.29 (m, 2H), 6.78-7.07 (m, 3H), 4.00-4.40 (m, 1H), 3.56-3.80 (m, 1H), 3.05-3.28 (m, 1H), 2.72-3.00 (m, 2H), 2.30-2.50 (m, 1H), 1.60-1.80 (m, 1H), 1.20-1.47 (m, 9H). LC-MS (ESI, m/z): 246 [M+H−Boc]⁺. t-butyl (3S,5S)-5-carbamoyl-2′-oxo-1′,4′-dihydro-2′H-spiro[pyrrolidine-3,3′-quinoline]-1-carboxylate (116.0 mg) as a white solid. ¹H NMR (400 MHz, DMSO-d₆) δ 10.34 (s, 1H), 7.31-7.50 (m, 1H), 7.07-7.28 (m, 2H), 6.80-7.01 (m, 3H), 4.02-4.33 (m, 1H), 3.44-3.60 (m, 1H), 3.20-3.42 (m, 1H), 2.82-3.00 (m, 2H), 2.10-2.30 (m, 1H), 1.90-2.08 (m, 1H), 1.23-1.48 (m, 9H). LC-MS (ESI, m/z): 346 [M+H]⁺.

A mixture of t-butyl (3S,5S)-5-carbamoyl-2′-oxo-1′,4′-dihydro-2′H-spiro[pyrrolidine-3,3′-quinoline]-1-carboxylate (100 mg, 0.290 mmol, 1.0 eq.) in HCl (2 mL, 4 M in 1,4-dioxane) was stirred for 1 h at rt. The mixture was concentrated under reduced pressure to afford (3S,5S)-2′-oxo-1′,4′-dihydro-2′H-spiro[pyrrolidine-3,3′-quinoline]-5-carboxamide (100 mg, crude) as a white solid. LC-MS (ESI, m/z): 246 [M+H]⁺.

To a stirred mixture of (3S,5S)-2′-oxo-1′,4′-dihydro-2′H-spiro[pyrrolidine-3,3′-quinoline]-5-carboxamide (100 mg, 0.408 mmol), N-methyl-N-((2,2,2-trifluoroacetyl)-L-alanyl)-L-leucine (131 mg, 0.420 mmol) and HATU (186 mg, 0.490 mmol) in DMF (2 mL) was added N-ethyl-N-isopropylpropan-2-amine (105 mg, 0.816 mmol). The mixture was stirred for 40 min at −15° C. and the reaction was quenched with water (5 mL). The mixture was extracted with EA (3×30 mL). The organic layers were combined, washed with brine (2×10 mL) and dried over anhydrous sodium sulfate. The solids were removed by filtration and the filtrate was concentrated under reduced pressure. The crude product was purified by TLC (Mobile phase: CH₃OH:DCM=1:9; Rf=0.4; detection: UV) to provide (3S,5S)-1-(N-methyl-N-((2,2,2-trifluoroacetyl)-L-alanyl)leucyl)-2′-oxo-1′,4′-dihydro-2′H-spiro[pyrrolidine-3,3′-quinoline]-5-carboxamide (85.0 mg, 39%) as a white solid. ¹H NMR (400 MHz, DMSO-d₆) δ 1.40 (br, 1H), 9.50-9.74 (m, 1H), 7.00-7.50 (m, 3H), 6.72-6.93 (m, 3H), 5.02-5.30 (m, 1H), 4.50-4.75 (m, 1H), 4.20-4.55 (m, 1H), 3.75-4.00 (m, 1H), 3.42-3.65 (m, 1H), 2.69-3.01 (m, 5H), 1.78-2.15 (m, 2H), 1.30-1.62 (m, 2H), 1.00-1.28 (m, 4H), 0.72-0.97 (m, 6H). LC-MS (ESI, m/z): 562 [M+Na]⁺.

To a mixture of (3S,5S)-1-(N-methyl-N-((2,2,2-trifluoroacetyl)-L-alanyl)-L-leucyl)-2′-oxo-1′,4′-dihydro-2′H-spiro[pyrrolidine-3,3′-quinoline]-5-carboxamide (80.0 mg, 0.148 mmol) in DCM (1 mL) were added pyridine (64.0 mg, 0.742 mmol) and trifluoroacetic anhydride (46.8 mg, 0.223 mmol). The mixture was stirred overnight at rt and the reaction was quenched with water (10 mL). The mixture was extracted with DCM (3×20 mL). The organic layers were combined, washed with brine (2×10 mL) and dried over anhydrous sodium sulfate. The solids were removed by filtration and the filtrate was concentrated under reduced pressure. The crude product was purified by prep-HPLC (Column: Xselect CSH Prep OBD C18 Column 30×150 mm, 5 m; Mobile Phase A: Water (0.1% FA), Mobile Phase B: ACN; Flow rate: 60 mL/min; Gradient: 40 to 55% B in 8 min; Wave Length: 220 nm) to provide (S)—N—((S)-1-((3S,5S)-5-cyano-2′-oxo-1′,4′-dihydro-2′H-spiro[pyrrolidine-3,3′-quinolin]-1-yl)-4-methyl-1-oxopentan-2-yl)-N-methyl-2-(2,2,2-trifluoroacetamido)propanamide (31.8 mg, 41%) as a white solid. ¹H NMR (400 MHz, 80° C., DMSO-d₆) δ 10.31 (s, 1H), 9.50 (br, 1H), 7.05-7.35 (m, 2H), 6.78-7.03 (m, 2H), 5.00-5.20 (m, 1H), 4.85-4.98 (m, 1H), 4.60-4.82 (m, 1H), 3.80-4.05 (m, 1H), 3.37-3.76 (m, 1H), 3.11-3.27 (m, 1H), 2.67-2.93 (m, 4H), 2.17-2.46 (m, 2H), 1.32-1.70 (m, 3H), 1.11-1.37 (m, 3H), 0.89-0.99 (m, 3H), 0.77-0.88 (m, 3H). LC-MS (ESI, m/z): 544 [M+Na]⁺;

### Example 33

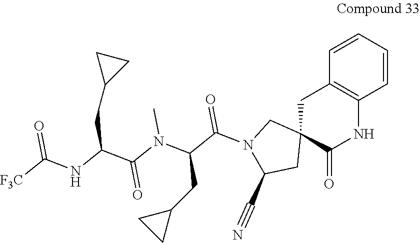 [SMILES: CN(C(=O)[C@H](CC1CC1)NC(=O)C(F)(F)F)[C@H](CC1CC1)C(=O)N1C[C@]2(Cc3ccccc3NC2=O)C[C@H]1C#N]

To a mixture of (3S,5S)-2′-oxo-1′,4′-dihydro-2′H-spiro[pyrrolidine-3,3′-quinoline]-5-carboxamide (60.0 mg, 0.245 mmol), (S)-3-cyclopropyl-2-((S)-3-cyclopropyl-N-methyl-2-(2,2,2-trifluoroacetamido)propanamido)propanoic acid (86.0 mg, 0.245 mmol) and HATU (111 mg, 0.294 mmol) in DMF (1 mL) was added N-ethyl-N-isopropylpropan-2-amine (63.0 mg, 0.490 mmol) at −15° C. The mixture was stirred for 30 min at −15° C. and the reaction was quenched with water (4 mL). The mixture was extracted with EA (3×4 mL). The organic layers were combined, washed with brine (2×4 mL) and dried over anhydrous sodium sulfate. The solids were removed by filtration and the filtrate was concentrated under reduced pressure. The crude product was chromatographed on a silica gel column with CH₃OH:DCM (3:97) to provide (3S,5S)-1-(3-cyclopropyl-2-((S)-3-cyclopropyl-N-methyl-2-(2,2,2-trifluoroacetamido)propanamido)propanoyl)-2′-oxo-1′,4′-dihydro-2′H-spiro[pyrrolidine-3,3′-quinoline]-5-carboxamide (90.0 mg, 63%) as a white solid. ¹H NMR (400 MHz, DMSO-d₆) δ 10.29-10.41 (m, 1H), 9.62-9.72 (m, 1H), 7.24-7.32 (m, 1H), 7.11-7.23 (m, 2H), 6.92-7.00 (m, 1H), 6.82-6.91 (m, 2H), 5.10-5.22 (m, 1H), 4.68-4.86 (m, 1H), 4.21-4.34 (m, 1H), 3.83-3.91 (m, 1H), 3.56-3.66 (m, 1H), 2.89-3.03 (m, 3H), 2.69-2.82 (m, 2H), 1.90-2.08 (m, 2H), 1.37-1.73 (m, 4H), 0.54-0.74 (m, 2H), 0.26-0.43 (m, 4H), −0.05-0.22 (m, 4H). LC-MS (ESI, m/z): 578 [M+H]⁺.

To a mixture of (3S,5S)-1-(3-cyclopropyl-2-((S)-3-cyclopropyl-N-methyl-2-(2,2,2-trifluoroacetamido)propanamido)propanoyl)-2′-oxo-1′,4′-dihydro-2′H-spiro[pyrrolidine-3,3′-quinoline]-5-carboxamide (90.0 mg, 0.156 mmol) in DCM (2 mL) were added pyridine (62.0 mg, 0.780 mmol) and trifluoroacetic anhydride (65.0 mg, 0.312 mmol). The mixture was stirred for 1 h at rt and the reaction was quenched with water (5 mL). The mixture was extracted with DCM (3×5 mL). The organic layers were combined, washed with brine (2×5 mL) and dried over anhydrous sodium sulfate. The solids were removed by filtration and the filtrate was concentrated under reduced pressure. The crude product was purified by prep-HPLC (Column: Xselect CSH Prep OBD C18 Column, 30×150 mm, 5 m; Mobile Phase A: Water (0.1% FA), Mobile Phase B: ACN; Flow rate: 60 mL/min; Gradient: 45% B to 50% B in 8 min; Wave Length: 220 nm) to provide (S)—N—((S)-1-((3S,5S)-5-cyano-2′-oxo-1′,4′-dihydro-2′H-spiro[pyrrolidine-3,3′-quinolin]-1-yl)-3-cyclopropyl-1-oxopropan-2-yl)-3-cyclopropyl-N-methyl-2-(2,2,2-trifluoroacetamido)propanamide (30.1 mg, 34%) as a white solid. ¹H NMR (400 MHz, 80° C., DMSO-d₆) δ 10.14 (s, 1H), 9.30 (br, 1H), 7.00-7.18 (m, 2H), 6.72-6.98 (m, 2H), 4.90-5.20 (m, 1H), 4.55-4.89 (m, 2H), 3.70-4.10 (m, 1H), 3.35-3.65 (m, 1H), 2.90-3.08 (m, 3H), 2.65-2.89 (m, 2H), 2.05-2.35 (m, 2H), 1.34-1.80 (m, 4H), 0.48-0.85 (m, 2H), 0.20-0.47 (m, 4H), −0.25-0.19 (m, 4H). LC-MS (ESI, m/z): 582 [M+Na]*.

### Example 34

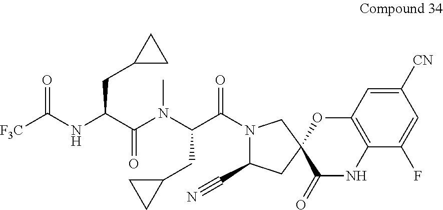 [SMILES: CN(C(=O)[C@H](CC1CC1)NC(=O)C(F)(F)F)[C@@H](CC1CC1)C(=O)N1C[C@@]2(C[C@H]1C#N)Oc1cc(C#N)cc(F)c1NC2=O]

To a mixture of 1-(t-butyl) 2-methyl (2S,4S)-4-hydroxy-4-(trichloromethyl)pyrrolidine-1,2-dicarboxylate (1.00 g, 2.76 mmol) and 5-bromo-3-fluoro-2-iodophenol (1.31 g, 4.14 mmol) in acetone (20 mL) was added NaOH (551 mg, 13.8 mmol) at 0° C. The mixture was stirred overnight at rt and the reaction was quenched with water (30 mL) and adjusted to pH=5 with HCl (1 M). The mixture was extracted with EA (3×40 mL). The organic layers were combined, washed with brine (2×30 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to provide (2S,4R)-4-(5-bromo-3-fluoro-2-iodophenoxy)-1-(t-butoxycarbonyl)pyrrolidine-2,4-dicarboxylic acid (820 mg, 49%) as a light yellow solid. ¹H NMR (400 MHz, DMSO-d₆) δ 13.54-14.71 (m, 1H), 11.91-13.53 (m, 1H), 7.22-7.41 (m, 1H), 6.61-6.75 (m, 1H), 4.26-4.46 (m, 1H), 3.91-4.15 (m, 1H), 3.68-3.81 (m, 1H), 2.74-2.91 (m, 1H), 2.40-2.48 (m, 1H), 1.20-1.35 (m, 9H). LC-MS (ESI, m/z): 474 [M−100+H]⁺.

To a mixture of (2S,4R)-4-(5-bromo-3-fluoro-2-iodophenoxy)-1-(t-butoxycarbonyl)pyrrolidine-2,4-dicarboxylic acid (820 mg, 1.43 mmol) and hydroxybenzotriazole (1.16 g, 8.57 mmol) in THF (20 mL) was added 1-(3-dimethylaminopropyl)-3-ethylcarbodiimide HCl (1.23 g, 6.43 mmol) at 0° C. After stirred for 30 min at 0° C., NH₄OH (15 mL, 30% in water) was added. The mixture was stirred for 2 h at rt and then diluted with water (30 mL). The mixture was extracted with EA (3×30 mL). The organic layers were combined, washed with brine (2×30 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to provide t-butyl (2S,4R)-4-(5-bromo-3-fluoro-2-iodophenoxy)-2,4-dicarbamoylpyrrolidine-1-carboxylate (510 mg, 53%) as a light-yellow solid. ¹H NMR (400 MHz, DMSO-d₆) δ 7.96-8.07 (m, 1H), 7.76 (s, 1H), 7.40-7.50 (m, 1H), 7.28-7.39 (m, 1H), 6.95-7.07 (m, 1H), 6.69 (s, 1H), 4.12-4.32 (m, 1H), 3.91-4.05 (m, 1H), 3.66-3.76 (m, 1H), 2.59-2.72 (m, 1H), 2.21-2.38 (m, 1H), 1.21-1.39 (m, 9H). LC-MS (ESI, m/z): 516 [M−56+H]⁺.

To a mixture of t-butyl (2S,4R)-4-(5-bromo-3-fluoro-2-iodophenoxy)-2,4-dicarbamoylpyrrolidine-1-carboxylate (510 mg, 0.891 mmol), copper(I) iodide (68.0 mg, 0.356 mmol) and cesium carbonate (583 mg, 1.78 mmol) in THF (10 mL) was added N,N′-dimethyl-1,2-ethanediamine (94.0 mg, 1.07 mmol). The mixture was stirred for 1 h at 70° C. under nitrogen and the reaction was quenched with water (20 mL). The mixture was extracted with EA (3×15 mL). The organic layers were combined, washed with brine (2×15 mL) and dried over anhydrous sodium sulfate. The solids were removed by filtration and the filtrate was concentrated under reduced pressure to provide t-butyl (2R,5'S)-7-bromo-5′-carbamoyl-5-fluoro-3-oxo-3,4-dihydrospiro[benzo[b][1,4]oxazine-2,3′-pyrrolidine]-1′-carboxylate (350 mg, 80%) as a light yellow solid. ¹H NMR (400 MHz, DMSO-d₆) δ 11.26 (s, 1H), 7.43-7.53 (m, 1H), 7.27-7.35 (m, 1H), 7.01-7.16 (m, 2H), 4.19-4.31 (m, 1H), 3.64-3.80 (m, 2H), 2.50-2.55 (m, 1H), 2.22-2.32 (m, 1H), 1.30-1.42 (m, 9H). LC-MS (ESI, m/z): 388 [M-56+H]⁺.

To a mixture of t-butyl (2R,5'S)-7-bromo-5′-carbamoyl-5-fluoro-3-oxo-3,4-dihydrospiro[benzo[b][1,4]oxazine-2,3′-pyrrolidine]-1′-carboxylate (350 mg, 0.788 mmol), zinc cyanide (111 mg, 0.946 mmol), tris(dibenylideneacetone)dipalladium (72.0 mg, 0.079 mmol) and 1,1′-bis(diphenylphosphino)ferrocene (87.0 mg, 0.158 mmol) in DMF (10 mL) was added zinc (36.0 mg, 0.552 mmol). The mixture was stirred for 1 h at 120° C. under nitrogen and the reaction was quenched with water (20 mL). The mixture was extracted with EA (3×20 mL). The organic layers were combined, washed with brine (2×20 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to provide t-butyl (2R,5'S)-5′-carbamoyl-7-cyano-5-fluoro-3-oxo-3,4-dihydrospiro[benzo[b][1,4]oxazine-2,3′-pyrrolidine]-1′-carboxylate (200 mg, 61%) as an off-white solid. ¹H NMR (400 MHz, DMSO-d₆) δ 11.65 (s, 1H), 7.60-7.66 (m, 1H), 7.41-7.54 (m, 2H), 7.01-7.15 (m, 1H), 4.21-4.31 (m, 1H), 3.75-3.82 (m, 1H), 3.67-3.74 (m, 1H), 2.51-2.58 (m, 1H), 2.23-2.33 (m, 1H), 1.34-1.40 (m, 9H). LC-MS (ESI, m/z): 335 [M-56+H]⁺.

To a solution of t-butyl (2R,5'S)-5′-carbamoyl-7-cyano-5-fluoro-3-oxo-3,4-dihydrospiro[benzo[b][1,4]oxazine-2,3′-pyrrolidine]-1′-carboxylate (100 mg, 0.256 mmol, 1.0 eq.) in DCM (3 mL) was added TFA (1 mL). The mixture was stirred for 1 h at rt and concentrated under reduced pressure to afford (2R,5'S)-7-cyano-5-fluoro-3-oxo-3,4-dihydrospiro[benzo[b][1,4]oxazine-2,3′-pyrrolidine]-5′-carboxamide (74.0 mg, crude) as a brown oil. LC-MS (ESI, m/z): 291 [M+H]⁺.

To a mixture of (2R,5'S)-7-cyano-5-fluoro-3-oxo-3,4-dihydrospiro[benzo[b][1,4]oxazine-2,3′-pyrrolidine]-5′-carboxamide (74.0 mg, 0.255 mmol, 1.0 eq.), (S)-3-cyclopropyl-2-((S)-3-cyclopropyl-N-methyl-2-(2,2,2-trifluoroacetamido)propanamido)propanoic acid (89.0 mg, 0.255 mmol) and HATU (165 mg, 0.433 mmol) in DMF (1 mL) was added N-ethyl-N-isopropylpropan-2-amine (99.0 mg, 0.765 mmol) at −15° C. The mixture was stirred for 40 mins at −15° C. and the reaction was quenched with water (5 mL). The mixture was extracted with EA (3×5 mL). The organic layers were combined, washed with brine (2×5 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to afford (2R,5'S)-7-cyano-1′-(3-cyclopropyl-2-((S)-3-cyclopropyl-N-methyl-2-(2,2,2-trifluoroacetamido)propanamido)propanoyl)-5-fluoro-3-oxo-3,4-dihydrospiro[benzo[b][1,4]oxazine-2,3′-pyrrolidine]-5′-carboxamide (140 mg, crude) as a light yellow solid. LC-MS (ESI, m/z): 645 [M+Na]⁺.

To a mixture of (2R,5'S)-7-cyano-1′-(3-cyclopropyl-2-((S)-3-cyclopropyl-N-methyl-2-(2,2,2-trifluoroacetamido)propanamido)propanoyl)-5-fluoro-3-oxo-3,4-dihydrospiro[benzo[b][1,4]oxazine-2,3′-pyrrolidine]-5′-carboxamide (140 mg, 0.225 mmol) in DCM (2 mL) were added pyridine (89.0 mg, 1.13 mmol) and trifluoroacetic anhydride (95.0 mg, 0.450 mmol). The mixture was stirred for 1 h at rt and the reaction was quenched with water (5 mL). The mixture was extracted with DCM (3×5 mL). The organic layers were combined, washed with brine (2×5 mL) and dried over anhydrous sodium sulfate. The solids were removed by filtration and the filtrate was concentrated under reduced pressure. The crude product was purified by prep-HPLC (Column: YMC-Actus Triart C18ExRs, 30×150 mm, 5 μm; Mobile Phase A: Water (10 mmol/L NH₄HCO₃+0.05% NH₃·H₂O), Mobile Phase B: ACN; Flow rate: 60 mL/min; Gradient: 36 to 66% B in 9 min; Wave Length: 254 nm/220 nm) to afford (S)-3-cyclopropyl-N—((S)-3-cyclopropyl-1-((2R,5'S)-5′,7-dicyano-5-fluoro-3-oxo-3,4-dihydrospiro[benzo[b][1,4]oxazine-2,3′-pyrrolidin]-1′-yl)-1-oxopropan-2-yl)-N-methyl-2-(2,2,2-trifluoroacetamido)propanamide (19.3 mg, 14%) as a white solid. ¹H NMR (400 MHz, 80° C., DMSO-d₆) δ 10.70-11.80 (m, 1H), 9.25 (br, 1H), 7.35-7.55 (m, 1H), 7.17 (s, 1H), 5.00-5.20 (m, 1H), 4.80-4.99 (m, 1H), 4.50-4.79 (m, 1H), 3.80-4.30 (m, 2H), 2.95 (s, 3H), 2.48-2.78 (m, 2H), 1.30-1.80 (m, 4H), 0.45-0.70 (m, 2H), 0.15-0.44 (m, 4H), −0.05-0.14 (m, 3H), −0.25-−0.04 (m, 1H). LC-MS (ESI, m/z): 627 [M+Na]⁺.

### Example 35

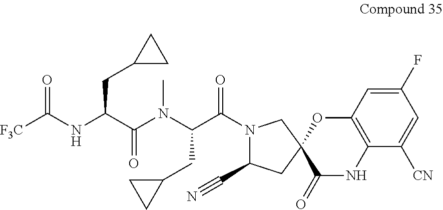 [SMILES: CN(C(=O)[C@H](CC1CC1)NC(=O)C(F)(F)F)[C@@H](CC1CC1)C(=O)N1C[C@@]2(C[C@H]1C#N)Oc1cc(F)cc(C#N)c1NC2=O]

To a mixture of 1-(t-butyl) 2-methyl (2S,4S)-4-hydroxy-4-(trichloromethyl)pyrrolidine-1,2-dicarboxylate (1.50 g, 4.136 mmol) and 3-bromo-5-fluoro-2-iodophenol (1.97 g, 6.20 mmol) in acetone (20 mL) was added NaOH (827 mg, 20.7 mmol) at 0° C. The mixture was stirred overnight at rt and the reaction was quenched with water (30 mL). The mixture was adjusted to pH=5 with HCl (1 M) and extracted with EA (3×30 mL). The organic layers were combined, washed with brine (2×30 mL) and dried over anhydrous sodium sulfate. The solids were removed by filtration and the filtrate was concentrated under reduced pressure. The crude product was purified by C18 column with CH₃CN/Water (0.05% FA). The fraction was concentrated under reduced pressure to provide (2S,4R)-4-(3-bromo-5-fluoro-2-iodophenoxy)-1-(t-butoxycarbonyl)pyrrolidine-2,4-dicarboxylic acid (1.47 g, crude) as a yellow oil. LC-MS (ESI, m/z): 518 [M−56+H]⁺.

To a mixture of (2S,4R)-4-(3-bromo-5-fluoro-2-iodophenoxy)-1-(t-butoxycarbonyl)pyrrolidine-2,4-dicarboxylic acid (1.50 g, 2.61 mmol) in THF (30 mL) were added 1-hydroxybenzotriazole (2.10 g, 15.7 mmol) and 1-(3-dimethylaminopropyl)-3-ethylcarbodiimide HCl (2.25 g, 11.7 mmol) at 0° C. After stirring for 30 mins at rt, NH₄OH (30 mL, 30% in water) was added at 0° C. The mixture was stirred for 2 h at rt. The mixture was diluted with water (50 mL) and extracted with EA (3×50 mL). The organic layers were combined, washed with brine (2×50 mL) and dried over anhydrous sodium sulfate. The solids were removed by filtration and the filtrate was concentrated under reduced pressure to afford the crude product that was purified by C18 column with CH₃CN/Water (0.05% TFA). The fraction was concentrated under reduced pressure to provide t-butyl (2S,4R)-4-(3-bromo-5-fluoro-2-iodophenoxy)-2,4-dicarbamoylpyrrolidine-1-carboxylate (770 mg, 41%) as a brown solid. ¹H NMR (400 MHz, DMSO-d₆) δ 7.93-8.03 (m, 1H), 7.74 (s, 1H), 7.40-7.54 (m, 2H), 6.98-7.10 (m, 1H), 6.46-6.62 (m, 1H), 4.12-4.32 (m, 1H), 3.97-4.05 (m, 1H), 3.66-3.74 (m, 1H), 2.62-2.72 (m, 1H), 2.20-2.36 (m, 1H), 1.23-1.39 (m, 9H). LC-MS (ESI, m/z): 516 [M-56+H]⁺.

To a mixture of t-butyl (2S,4R)-4-(3-bromo-5-fluoro-2-iodophenoxy)-2,4-dicarbamoylpyrrolidine-1-carboxylate (770 mg, 1.35 mmol), cuprous iodide (103 mg, 0.538 mmol) and cesium carbonate (877 mg, 2.70 mmol) in THF (10 mL) was added N,N′-dimethyl-1,2-ethanediamine (142 mg, 1.61 mmol). The resulting mixture was stirred for 1 h at 70° C. under nitrogen and the reaction was quenched with water (20 mL). The mixture was extracted with EA (3×20 mL). The organic layers were combined, washed with brine (2×20 mL) and dried over anhydrous sodium sulfate. The solids were removed by filtration and the filtrate was concentrated under reduced pressure to provide t-butyl (2R,5'S)-5-bromo-5′-carbamoyl-7-fluoro-3-oxo-3,4-dihydrospiro[benzo[b][1,4]oxazine-2,3′-pyrrolidine]-1′-carboxylate (515 mg, 62%) as a light yellow semi-solid. ¹H NMR (400 MHz, DMSO-d₆) δ 10.55 (br, 1H), 7.45-7.55 (m, 1H), 7.30-7.37 (m, 1H), 6.99-7.17 (m, 2H), 4.21-4.31 (m, 1H), 3.65-3.78 (m, 2H), 2.42-2.49 (m, 1H), 2.27-2.35 (m, 1H), 1.35-1.42 (m, 9H). LC-MS (ESI, m/z): 388 [M-56+H]+

To a mixture of t-butyl (2R,5'S)-5-bromo-5′-carbamoyl-7-fluoro-3-oxo-3,4-dihydrospiro[benzo[b][1,4]oxazine-2,3′-pyrrolidine]-1′-carboxylate (560 mg, 1.26 mmol, 1.0 eq.), zinc cyanide (178 mg, 1.51 mmol), tris(dibenzylideneacetone)dipalladium (115 mg, 0.126 mmol) and zinc (58.0 mg, 0.883 mmol) in THF (6 mL) was added 1,1′-Bis(diphenylphosphino)ferrocene (140 mg, 0.252 mmol) under nitrogen. The mixture was stirred for 1 h at 120° C. and the reaction was quenched with water (10 mL). The mixture was extracted with EA (3×10 mL). The organic layers were combined, washed with brine (2×10 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to provide t-butyl (2R,5'S)-5′-carbamoyl-5-cyano-7-fluoro-3-oxo-3,4-dihydrospiro[benzo[b][1,4]oxazine-2,3′-pyrrolidine]-1′-carboxylate (150 mg, 28%) as a yellow semi-solid. ¹H NMR (400 MHz, DMSO-d₆) δ 11.48 (s, 1H), 7.39-7.78 (m, 3H), 6.99-7.16 (m, 1H), 4.20-4.32 (m, 1H), 3.61-3.81 (m, 2H), 2.52-2.58 (m, 1H), 2.23-2.36 (m, 1H), 1.21-1.43 (m, 9H). LC-MS (ESI, m/z): 335 [M-56+H]⁺.

To a mixture of t-butyl (2R,5'S)-5′-carbamoyl-5-cyano-7-fluoro-3-oxo-3,4-dihydrospiro[benzo[b][1,4]oxazine-2,3′-pyrrolidine]-1′-carboxylate (150 mg, 0.385 mmol, 1.0 eq.) in DCM (3 mL) was added TFA (1 mL). The mixture was stirred for 1 h at rt and concentrated under reduced pressure to afford (2R,5'S)-5-cyano-7-fluoro-3-oxo-3,4-dihydrospiro[benzo[b][1,4]oxazine-2,3′-pyrrolidine]-5′-carboxamide (112 mg, crude) as a brown oil. LC-MS (ESI, m/z): 313 [M+Na]⁺.

To a mixture of (2R,5'S)-5-cyano-7-fluoro-3-oxo-3,4-dihydrospiro[benzo[b][1,4]oxazine-2,3′-pyrrolidine]-5′-carboxamide (112 mg, 0.385 mmol), (S)-3-cyclopropyl-2-((S)-3-cyclopropyl-N-methyl-2-(2,2,2-trifluoroacetamido)propanamido)propanoic acid (135 mg, 0.385 mmol) and HATU (219 mg, 0.577 mmol) in DMF (2 mL) was added N-ethyl-N-isopropylpropan-2-amine (149 mg, 1.15 mmol) at −15° C. The mixture was stirred for 40 mins at −15° C. and the reaction was quenched with water (5 mL). The mixture was extracted with EA (3×5 mL). The organic layers were combined, washed with brine (2×5 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to afford (2R,5'S)-5-cyano-1′-(3-cyclopropyl-2-((S)-3-cyclopropyl-N-methyl-2-(2,2,2-trifluoroacetamido)propanamido)propanoyl)-7-fluoro-3-oxo-3,4-dihydrospiro[benzo[b][1,4]oxazine-2,3′-pyrrolidine]-5′-carboxamide (210 mg, crude) as a light yellow solid. LC-MS (ESI, m/z): 645 [M+Na]⁺.

To a mixture of (2R,5'S)-5-cyano-1′-(3-cyclopropyl-2-((S)-3-cyclopropyl-N-methyl-2-(2,2,2-trifluoroacetamido)propanamido)propanoyl)-7-fluoro-3-oxo-3,4-dihydrospiro[benzo[b][1,4]oxazine-2,3′-pyrrolidine]-5′-carboxamide (210 mg, 0.337 mmol) in DCM (2 mL) were added pyridine (133 mg, 1.68 mmol) and trifluoroacetic anhydride (142 mg, 0.674 mmol). The mixture was stirred for 1 h at rt and the reaction was quenched with water (5 mL). The mixture was extracted with DCM (3×5 mL). The organic layers were combined, washed with brine (2×5 mL) and dried over anhydrous sodium sulfate. The solids were removed by filtration and the filtrate was concentrated under reduced pressure. The crude product was purified by prep-HPLC (Column: YMC-Actus Triart C18ExRs, 30×150 mm, 5 m; Mobile Phase A: Water (10 mmol/L NH₄HCO₃+0.05% NH₃·H₂O), Mobile Phase B: ACN; Flow rate: 60 mL/min; Gradient: 36% B to 66% B in 9 min; Wave Length: 254 nm/220 nm) to provide isomer 1: (S)-3-cyclopropyl-N—((S)-3-cyclopropyl-1-((2R,5'S)-5,5′-dicyano-7-fluoro-3-oxo-3,4-dihydrospiro[benzo[b][1,4]oxazine-2,3′-pyrrolidin]-1′-yl)-1-oxopropan-2-yl)-N-methyl-2-(2,2,2-trifluoroacetamido)propanamide (43.3 mg, 21%) as a white solid. ¹H NMR (400 MHz, 80° C., DMSO-d₆) δ 11.17 (br, 1H), 8.86-9.59 (m, 1H), 7.22-7.42 (m, 1H), 6.91-7.20 (m, 1H), 4.98-5.28 (m, 1H), 4.78-4.96 (m, 1H), 4.49-4.76 (m, 1H), 3.85-4.25 (m, 2H), 2.94 (s, 3H), 2.48-2.78 (m, 2H), 1.31-1.76 (m, 4H), 0.48-0.81 (m, 2H), 0.18-0.46 (m, 4H), −0.07-0.17 (m, 3H), −0.29-−0.11 (m, 1H). LC-MS (ESI, m/z): 627 [M+Na]⁺;

### Example 36

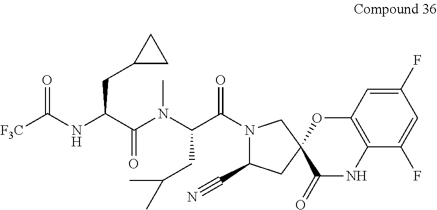 [SMILES: CC(C)C[C@@H](C(=O)N1C[C@@]2(C[C@H]1C#N)Oc1cc(F)cc(F)c1NC2=O)N(C)C(=O)[C@H](CC1CC1)NC(=O)C(F)(F)F]

To a mixture of (2S)-2-[(t-butoxycarbonyl)amino]-3-cyclopropylpropanoic acid (1.00 g, 4.36 mmol), methyl methyl-L-leucinate HCl (852 mg, 4.36 mmol) and HATU (1.99 g, 5.23 mmol) in DMF (10 mL) was added N-ethyl-N-isopropylpropan-2-amine (2.25 g, 17.4 mmol) at 0° C. The mixture was stirred for 1 h at rt and the reaction was quenched with water (20 mL). The mixture was extracted with EA (3×20 mL). The organic layers were combined, washed with brine (2×20 mL) and dried over anhydrous sodium sulfate. The solids were removed by filtration and the filtrate was concentrated under reduced pressure. The crude product was chromatographed on a silica gel column with EA:PE (1:6) to provide methyl (2S)-2-[(2S)-2-[(t-butoxycarbonyl)amino]-3-cyclopropyl-N-methylpropanamido]-4-methylpentanoate (1.4 g, 85%) as a light yellow oil. ¹H NMR (400 MHz, DMSO-d₆) δ 7.04 (d, J=7.9 Hz, 1H), 5.16-5.24 (m, 1H), 4.36-4.47 (m, 1H), 3.56-3.64 (m, 3H), 2.90-3.04 (m, 3H), 1.70-1.81 (m, 1H), 1.39-1.57 (m, 4H), 1.33-1.38 (m, 9H), 0.77-0.94 (m, 6H), 0.67-0.76 (m, 1H), 0.32-0.45 (m, 2H), 0.00-0.19 (m, 2H). LC-MS (ESI, m/z): 271 [M−Boc+H]⁺.

To a solution of methyl (2S)-2-[(2S)-2-[(t-butoxycarbonyl)amino]-3-cyclopropyl-N-methylpropanamido]-4-methylpentanoate (1.40 g, 3.78 mmol) in THF (15 mL) was added a solution of lithium hydroxide (272 mg, 11.3 mmol) in water (15 mL). The mixture was stirred for 1 h at rt and then diluted with water (30 mL). The mixture was adjusted to pH=5 with HCl (1 M) and extracted with EA (3×30 mL). The organic layers were combined, washed with brine (2×30 mL) and dried over anhydrous sodium sulfate. The solids were removed by filtration and the filtrate was concentrated under reduced pressure to provide (2S)-2-[(2S)-2-[(t-butoxycarbonyl)amino]-3-cyclopropyl-N-methylpropanamido]-4-methylpentanoic acid (1.20 g, 88%) as a light yellow oil. ¹H NMR (400 MHz, DMSO-d₆) δ 12.65 (br, 1H), 6.95-7.10 (m, 1H), 5.10-5.18 (m, 1H), 4.37-4.47 (m, 1H), 2.88-3.02 (m, 3H), 1.67-1.78 (m, 1H), 1.40-1.58 (m, 4H), 1.33-1.37 (m, 9H), 0.76-0.90 (m, 6H), 0.68-0.75 (m, 1H), 0.33-0.43 (m, 2H), 0.01-0.18 (m, 2H). LC-MS (ESI, m/z): 357 [M+H]⁺.

To a mixture of (2S)-2-[(2S)-2-[(t-butoxycarbonyl)amino]-3-cyclopropyl-N-methylpropanamido]-4-methylpentanoic acid (1.40 g, 3.93 mmol) in DCM (15 mL) was added TFA (5 mL). The mixture was stirred for 1 h at rt and concentrated under reduced pressure to afford (2S)-2-[(2S)-2-amino-3-cyclopropyl-N-methylpropanamido]-4-methylpentanoic acid (1.00 g, crude) as a brown oil. LC-MS (ESI, m/z): 257 [M+H]⁺.

To a mixture of (2S)-2-[(2S)-2-amino-3-cyclopropyl-N-methylpropanamido]-4-methylpentanoic acid (1.00 g, 3.90 mmol) in CH₃OH (10 mL) were added triethylamine (4.74 g, 46.8 mmol) and ethyl 2,2,2-trifluoroacetate (5.50 g, 39.0 mmol). The mixture was stirred overnight at rt and concentrated under reduced pressure to remove the solvent. The residue was diluted with water (20 mL) and adjusted to pH=5 with HCl (1 M). The mixture was extracted with EA (3×20 mL). The organic layers were combined, washed with brine (2×20 mL) and dried over anhydrous sodium sulfate. The solids were removed by filtration and the filtrate was concentrated under reduced pressure to provide (2S)-2-[(2S)-3-cyclopropyl-N-methyl-2-(2,2,2-trifluoroacetamido)propanamido]-4-methylpentanoic acid (1.2 g, 85%) as a light yellow oil. ¹H NMR (400 MHz, DMSO-d₆) δ 12.73 (br, 1H), 9.67-9.92 (m, 1H), 5.00-5.10 (m, 1H), 4.76-4.86 (m, 1H), 2.98 (s, 3H), 1.68-1.76 (m, 1H), 1.52-1.67 (m, 3H), 1.34-1.44 (m, 1H), 0.71-0.90 (m, 7H), 0.36-0.44 (m, 2H), 0.05-0.25 (m, 2H). LC-MS (ESI, m/z): 353 [M+H]⁺.

To a mixture of 1-t-butyl 2-methyl (2S,4S)-4-hydroxy-4-(trichloromethyl)pyrrolidine-1,2-dicarboxylate (12.0 g, 33.1 mmol) and 2-bromo-3,5-difluorophenol (13.8 g, 66.2 mmol) in acetone (150 mL) was added NaOH (8.00 g, 199 mmol) at 0° C. The mixture was stirred overnight at rt and the reaction was quenched with water (150 mL). The mixture was adjusted to pH=5 with HCl (aq., 1 M) and extracted with EA (3×150 mL). The organic layers were combined, washed with brine (2×100 mL) and dried over anhydrous sodium sulfate. The solids were removed by filtration and the filtrate was concentrated under reduced pressure to remove the solvent. The crude product was purified by C18 column with CH₃CN/Water (0.05% TFA). The fraction was concentrated under reduced pressure to afford (2S,4R)-4-(2-bromo-3,5-difluorophenoxy)-1-(t-butoxycarbonyl)pyrrolidine-2,4-dicarboxylic acid (7.0 g, crude) as a brown oil. LC-MS (ESI, m/z): 410 [M-56+H]⁺.

To a mixture of (2S,4R)-4-(2-bromo-3,5-difluorophenoxy)-1-(t-butoxycarbonyl)pyrrolidine-2,4-dicarboxylic acid (7.00 g, 15.0 mmol) in THF (80 mL) were added 1-hydroxybenzotriazole (10.0 g, 75.1 mmol) and 1-(3-dimethylaminopropyl)-3-ethylcarbodiimide HCl (11.5 g, 60.0 mmol) at 0° C. After stirring for 30 mins at rt, NH₄OH (80 mL, 30% in water) was added at 0° C. The mixture was stirred for 2 h at rt and then diluted with water (100 mL). The mixture was extracted with EA (3×100 mL). The organic layers were combined, washed with brine (2×100 mL) and dried over anhydrous sodium sulfate. The solids were removed by filtration and the filtrate was concentrated under reduced pressure. The crude product was chromatographed on a silica gel column with CH₃OH:DCM (8:92) to provide t-butyl (2S,4R)-4-(2-bromo-3,5-difluorophenoxy)-2,4-dicarbamoylpyrrolidine-1-carboxylate (1.2 g, 16%) as a light-yellow solid. ¹H NMR (400 MHz, DMSO-d₆) δ 8.00-8.10 (m, 1H), 7.76 (s, 1H), 7.40-7.52 (m, 1H), 7.19-7.31 (m, 1H), 6.98-7.09 (m, 1H), 6.40-6.60 (m, 1H), 4.08-4.25 (m, 1H), 3.95-4.06 (m, 1H), 3.64-3.72 (m, 1H), 2.60-2.72 (m, 1H), 2.24-2.36 (m, 1H), 1.31-1.38 (m, 9H). LC-MS (ESI, m/z): 408 [M-56+H]⁺.

To a mixture of t-butyl (2S,4R)-4-(2-bromo-3,5-difluorophenoxy)-2,4-dicarbamoylpyrrolidine-1-carboxylate (1.2 g, 2.58 mmol), methanesulfonato(2-dicyclohexylphosphino-2′,4′,6′-tri-1-propyl-1,1′-biphenyl)(2′-amino-1,1′-biphenyl-2-yl)palladium(II) (219 mg, 0.259 mmol) and 2-(dicyclohexylphosphino)-2′,4′,6′-triisopropylbiphenyl (123 mg, 0.259 mmol) in dioxane (15 mL) was added cesium carbonate (1.80 g, 5.62 mmol). The mixture was stirred overnight at 90° C. under nitrogen and the reaction was quenched with water (50 mL). The mixture was extracted with EA (3×50 mL). The organic layers were combined, washed with brine (2×50 mL) and dried over anhydrous sodium sulfate. The solids were removed by filtration and the filtrate was concentrated under reduced pressure. The crude product was chromatographed on a silica gel column with CH₃OH:DCM (1:25) to provide t-butyl (2R,5'S)-5′-carbamoyl-5,7-difluoro-3-oxo-4H-spiro[1,4-benzoxazine-2,3′-pyrrolidine]-1′-carboxylate (670 mg, 64%) as a light-yellow solid.

¹H NMR (400 MHz, DMSO-d₆) δ 11.20 (s, 1H), 7.49-7.60 (m, 1H), 6.99-7.18 (m, 2H), 6.80-6.94 (m, 1H), 4.17-4.32 (m, 1H), 3.64-3.81 (m, 2H), 2.50-2.55 (m, 1H), 2.22-2.36 (m, 1H), 1.37 (s, 9H). LC-MS (ESI, m/z): 328 [M-56+H]+

To a mixture of t-butyl (2R,5'S)-5′-carbamoyl-5,7-difluoro-3-oxo-4H-spiro[1,4-benzoxazine-2,3′-pyrrolidine]-1′-carboxylate (120 mg, 0.313 mmol) in DCM (3 mL) was added TFA (1 mL). The mixture was stirred for 1 h at rt and concentrated under reduced pressure to afford (2R,5'S)-5,7-difluoro-3-oxo-4H-spiro[1,4-benzoxazine-2,3′-pyrrolidine]-5′-carboxamide (89.0 mg, crude) as a light-yellow solid. LC-MS (ESI, m/z): 306 [M+Na]⁺.

To a mixture of (2R,5'S)-5,7-difluoro-3-oxo-4H-spiro[1,4-benzoxazine-2,3′-pyrrolidine]-5′-carboxamide (89.0 mg, 0.313 mmol), (2S)-2-[(2S)-3-cyclopropyl-N-methyl-2-(2,2,2-trifluoroacetamido)propanamido]-4-methylpentanoic acid (111 mg, 0.313 mmol) and HATU (144 mg, 0.376 mmol) in DMF (2 mL) was added N-ethyl-N-isopropylpropan-2-amine (61.0 mg, 0.471 mmol) at −15° C. The mixture was stirred for 1 h at −15° C. and the reaction was quenched with water (5 mL). The mixture was extracted with EA (3×5 mL). The organic layers were combined, washed with brine (2×5 mL) and dried over anhydrous sodium sulfate. The solids were removed by filtration and the filtrate was concentrated under reduced pressure. The crude product was chromatographed on a silica gel column with CH₃OH:DCM (1:20) to provide (2R,5'S)-1′-(N—((S)-3-cyclopropyl-2-(2,2,2-trifluoroacetamido)propanoyl)-N-methylleucyl)-5,7-difluoro-3-oxo-3,4-dihydrospiro[benzo[b][1,4]oxazine-2,3′-pyrrolidine]-5′-carboxamide (75.0 mg, 37%) as an off-white solid. ¹H NMR (400 MHz, DMSO-d₆) δ 11.20-11.30 (m, 1H), 9.67-9.75 (m, 1H), 7.46 (s, 1H), 6.97-7.08 (m, 2H), 6.80-6.86 (m, 1H), 5.16-5.26 (m, 1H), 4.65-4.75 (m, 1H), 4.30-4.39 (m, 1H), 4.01-4.10 (m, 2H), 2.94-2.99 (m, 3H), 2.46-2.49 (m, 1H), 2.05-2.15 (m, 1H), 1.51-1.55 (m, 1H), 1.34-1.42 (m, 2H), 1.23-1.27 (m, 2H), 0.85-0.90 (m, 3H), 0.79-0.83 (m, 3H), 0.56-0.68 (m, 1H), 0.26-0.35 (m, 2H), −0.12-−0.01 (m, 2H). LC-MS (ESI, m/z): 640 [M+Na]⁺.

To a mixture of (2R,5′S)-1′-(N—((S)-3-cyclopropyl-2-(2,2,2-trifluoroacetamido)propanoyl)-N-methylleucyl)-5,7-difluoro-3-oxo-3,4-dihydrospiro[benzo[b][1,4]oxazine-2,3′-pyrrolidine]-5′-carboxamide (75.0 mg, 0.121 mmol, 1.0 eq.) in DCM (1 mL) were added pyridine (48.0 mg, 0.605 mmol) and trifluoroacetic anhydride (51.0 mg, 0.242 mmol). The mixture was stirred for 1 h at rt and the reaction was quenched with water (5 mL). The mixture was extracted with DCM (3×5 mL). The organic layers were combined, washed with brine (2×5 mL) and dried over anhydrous sodium sulfate. The solids were removed by filtration and the filtrate was concentrated under reduced pressure to afford the crude product, that was purified by prep-HPLC (Column: Xselect CSH Prep OBD C18 Column, 30×150 mm, 5 m; Mobile Phase A: Water (0.1% FA), Mobile Phase B: ACN; Flow rate: 60 mL/min; Gradient: 52% B to 67% B in 8 min; Wave Length: 254 nm/220 nm) to provide: (S)—N—((S)-1-((2R,5'S)-5′-cyano-5,7-difluoro-3-oxo-3,4-dihydrospiro[benzo[b][1,4]oxazine-2,3′-pyrrolidin]-1′-yl)-4-methyl-1-oxopentan-2-yl)-3-cyclopropyl-N-methyl-2-(2,2,2-trifluoroacetamido)propanamide (32.2 mg, 43%) as a white solid. ¹H NMR (400 MHz, 80° C., DMSO-d₆) δ 10.55-11.80 (m, 1H), 9.17-9.88 (m, 1H), 6.91-7.32 (m, 1H), 6.53-6.90 (m, 1H), 5.16-5.49 (m, 1H), 4.91-5.14 (m, 1H), 4.51-4.90 (m, 1H), 3.88-4.42 (m, 2H), 2.90-3.10 (m, 2H), 2.61-2.89 (m, 3H), 1.33-1.90 (m, 5H), 0.78-1.31 (m, 6H), 0.52-0.76 (m, 1H), 0.21-0.50 (m, 2H), −0.82-0.20 (m, 2H). LC-MS (ESI, m/z): 622 [M+Na]⁺.

### Example 37

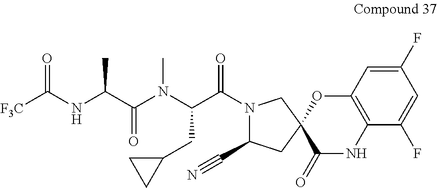 [SMILES: C[C@H](NC(=O)C(F)(F)F)C(=O)N(C)[C@@H](CC1CC1)C(=O)N1C[C@@]2(C[C@H]1C#N)Oc1cc(F)cc(F)c1NC2=O]

To a mixture of (2R,5'S)-5,7-difluoro-3-oxo-3,4-dihydrospiro[benzo[b][1,4]oxazine-2,3′-pyrrolidine]-5′-carboxamide (148 mg, 0.523 mmol), (S)-2-((t-butoxycarbonyl)(methyl)amino)-3-cyclopropylpropanoic acid (127 mg, 0.523 mmol) and HATU (238 mg, 0.628 mmol) in DMF (4 mL) was added N-ethyl-N-isopropylpropan-2-amine (270 mg, 2.09 mmol) at 0° C. The mixture was stirred for 1 h at rt and the reaction was quenched with water (10 mL). The mixture was extracted with EA (3×10 mL). The organic layers were combined, washed with brine (2×10 mL) and dried over anhydrous sodium sulfate. The solids were removed by filtration and the filtrate was concentrated under reduced pressure. The crude product was chromatographed on a silica gel column with CH₃OH:DCM (1:25) to provide t-butyl ((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)carbamate (150 mg, 51%) as a yellow semi-solid. ¹H NMR (400 MHz, DMSO-d₆) δ 11.21-11.31 (m, 1H), 7.48 (s, 1H), 6.97-7.14 (m, 2H), 6.73-6.85 (m, 1H), 4.60-4.77 (m, 1H), 4.32-4.42 (m, 1H), 3.74-4.04 (m, 2H), 3.32 (s, 3H), 2.52-2.58 (m, 1H), 2.38-2.48 (m, 1H), 1.47-1.63 (m, 1H), 1.38-1.44 (m, 1H), 1.19-1.28 (m, 9H), 0.46-0.61 (m, 1H). 0.25-0.43 (m, 2H), 0.00-0.15 (m, 2H). LC-MS (ESI, m/z): 531 [M+Na]⁺.

To a mixture of t-butyl ((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)carbamate (150 mg, 0.295 mmol) in DCM (3 mL) was added TFA (1 mL). The mixture was stirred for 1 h at rt and concentrated under reduced pressure to afford (2R,5'S)-1′-((S)-3-cyclopropyl-2-(methylamino)propanoyl)-5,7-difluoro-3-oxo-3,4-dihydrospiro[benzo[b][1,4]oxazine-2,3′-pyrrolidine]-5′-carboxamide (120 mg, crude) as a brown oil. LC-MS (ESI, m/z): 409 [M+H]⁺.

To a mixture of (2R,5'S)-1′-((S)-3-cyclopropyl-2-(methylamino)propanoyl)-5,7-difluoro-3-oxo-3,4-dihydrospiro[benzo[b][1,4]oxazine-2,3′-pyrrolidine]-5′-carboxamide (120 mg, 0.294 mmol, 1.0 eq.), t-butoxycarbonyl)-L-alanine (56.0 mg, 0.294 mmol) and HATU (134 mg, 0.353 mmol) in DMF (2 mL) was added N-ethyl-N-isopropylpropan-2-amine (152 mg, 1.18 mmol) at 0° C. The mixture was stirred for 1 h at rt and the reaction was quenched with water (5 mL). The mixture was extracted with EA (3×5 mL). The organic layers were combined, washed with brine (2×5 mL) and dried over anhydrous sodium sulfate. The solids were removed by filtration and the filtrate was concentrated under reduced pressure. The crude product was purified by C18 column with CH₃CN/Water (0.05% TFA). The fraction was concentrated under reduced pressure to provide t-butyl ((S)-1-(((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-1-oxopropan-2-yl)carbamate (160 mg, 80%) as a brown semi-solid. ¹H NMR (400 MHz, DMSO-d₆) δ 11.27 (s, 1H), 7.45 (s, 1H), 6.98-7.12 (m, 2H), 6.78-6.97 (m, 2H), 5.15-5.24 (m, 1H), 4.27-4.40 (m, 2H), 3.88-3.98 (m, 2H), 2.92 (s, 3H), 2.53-2.56 (m, 1H), 2.18-2.27 (m, 1H), 1.43-1.61 (m, 2H), 1.23-1.34 (m, 9H), 0.87-0.95 (m, 3H), 0.55-0.69 (m, 1H), 0.22-0.40 (m, 2H), 0.01-0.07 (m, 2H). LC-MS (ESI, m/z): 602 [M+Na]⁺.

To a mixture of t-butyl ((S)-1-(((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-1-oxopropan-2-yl)carbamate (160 mg, 0.276 mmol) in DCM (3 mL) was added TFA (1 mL). The mixture was stirred for 1 h at rt and concentrated under reduced pressure to afford (2R,5'S)-1′-((S)-2-((S)-2-amino-N-methylpropanamido)-3-cyclopropylpropanoyl)-5,7-difluoro-3-oxo-3,4-dihydrospiro[benzo[b][1,4]oxazine-2,3′-pyrrolidine]-5′-carboxamide (133 mg, crude) as a brown oil. LC-MS (ESI, m/z): 502 [M+Na]⁺.

To a mixture of (2R,5'S)-1′-((S)-2-((S)-2-amino-N-methylpropanamido)-3-cyclopropylpropanoyl)-5,7-difluoro-3-oxo-3,4-dihydrospiro[benzo[b][1,4]oxazine-2,3′-pyrrolidine]-5′-carboxamide (133 mg, 0.276 mmol) in MeOH (2 mL) were added trimethylamine (337 mg, 3.32 mmol) and ethyl 2,2,2-trifluoroacetate (394 mg, 2.77 mmol). The mixture was stirred overnight at rt and the reaction was quenched with water (5 mL). The mixture was adjusted to pH 5-6 with HCl (1 M). The mixture was extracted with EA (3×5 mL). The organic layers were combined, washed with brine (2×5 mL) and dried over anhydrous sodium sulfate. The solids were removed by filtration and the filtrate was concentrated under reduced pressure to provide (2R,5'S)-1′-((S)-3-cyclopropyl-2-((S)—N-methyl-2-(2,2,2-trifluoroacetamido)propanamido)propanoyl)-5,7-difluoro-3-oxo-3,4-dihydrospiro[benzo[b][1,4]oxazine-2,3′-pyrrolidine]-5′-carboxamide (100 mg, 60%) as a light yellow solid. ¹H NMR (400 MHz, DMSO-d₆) δ 11.28 (br, 1H), 9.56-9.72 (m, 1H), 7.46 (s, 1H), 7.01-7.10 (m, 2H), 6.87-6.93 (m, 1H), 5.15-5.24 (m, 1H), 4.63-4.77 (m, 1H), 4.32-4.43 (m, 1H), 3.91-4.03 (m, 2H), 2.79-2.98 (m, 3H), 2.53-2.59 (m, 1H), 2.15-2.25 (m, 1H), 1.60-1.67 (m, 1H), 1.45-1.51 (m, 1H), 1.23-1.27 (m, 3H), 0.55-0.63 (m, 1H), 0.22-0.40 (m, 2H), 0.02-0.08 (m, 2H). LC-MS (ESI, m/z): 598 [M+Na]⁺.

To mixture of (2R,5'S)-1′-((S)-3-cyclopropyl-2-((S)—N-methyl-2-(2,2,2-trifluoroacetamido)propanamido)propanoyl)-5,7-difluoro-3-oxo-3,4-dihydrospiro[benzo[b][1,4]oxazine-2,3′-pyrrolidine]-5′-carboxamide (90.0 mg, 0.156 mmol) in DCM (1 mL) were added pyridine (62.0 mg, 0.780 mmol) and trifluoroacetic anhydride (66.0 mg, 0.312 mmol). The mixture was stirred for 1 h at rt and the reaction was quenched with water (5 mL). The mixture was extracted with DCM (3×5 mL). The organic layers were combined, washed with brine (2×5 mL) and dried over anhydrous sodium sulfate. The solids were removed by filtration and the filtrate was concentrated under reduced pressure. The crude product was purified by prep-HPLC (Column: Xselect CSH Prep C18 Column, 30×150 mm, m; Mobile Phase A: Water (0.1% FA), Mobile Phase B: ACN; Flow rate: 60 mL/min; Gradient: 38 to 59% B in 10 min; Wave Length: 254 nm/220 nm) to provide (S)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-N-methyl-2-(2,2,2-trifluoroacetamido)propanamide (26.9 mg, 30%) as a white solid. ¹H NMR (400 MHz, 80° C., DMSO-d₆) δ 10.35-11.75 (m, 1H), 9.25 (br, 1H), 6.79-7.04 (m, 1H), 6.48-6.76 (m, 1H), 4.76-5.31 (m, 2H), 4.42-4.74 (m, 1H), 3.76-4.18 (m, 2H), 2.89 (s, 3H), 2.51-2.79 (m, 2H), 1.33-1.81 (m, 2H), 0.81-1.31 (m, 3H), 0.41-0.67 (m, 1H), 0.13-0.39 (m, 2H), −0.08-0.11 (m, 2H). LC-MS (ESI, m/z): 580 [M+Na]⁺.

### Example 38

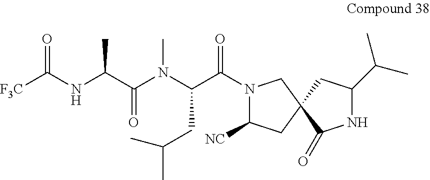

Compound 38 was prepared similarly as described for compound 21 using 2-(bromomethyl)-3-methylbut-1-ene in place of 3-bromo-2-methylprop-1-ene. ¹H NMR (500 MHz, 363K, DMSO-d₆) δ 9.25 (br. s., 1H), 7.84 (m, 1H), 5.17 (m, 1H), 4.76 (m, 2H), 3.39-3.84 (m, 2H), 3.18-3.33 (m, 1H), 2.97 (s, 3H), 2.38-2.45 (m, 1H), 2.13-2.35 (m, 1H), 1.89-2.13 (m, 1H), 1.44-1.70 (m, 5H), 1.31 (m, 3H), 0.81-0.93 (m, 12H). LC-MS (ESI, m/z): 500 [M−H]⁻.

### Example 39

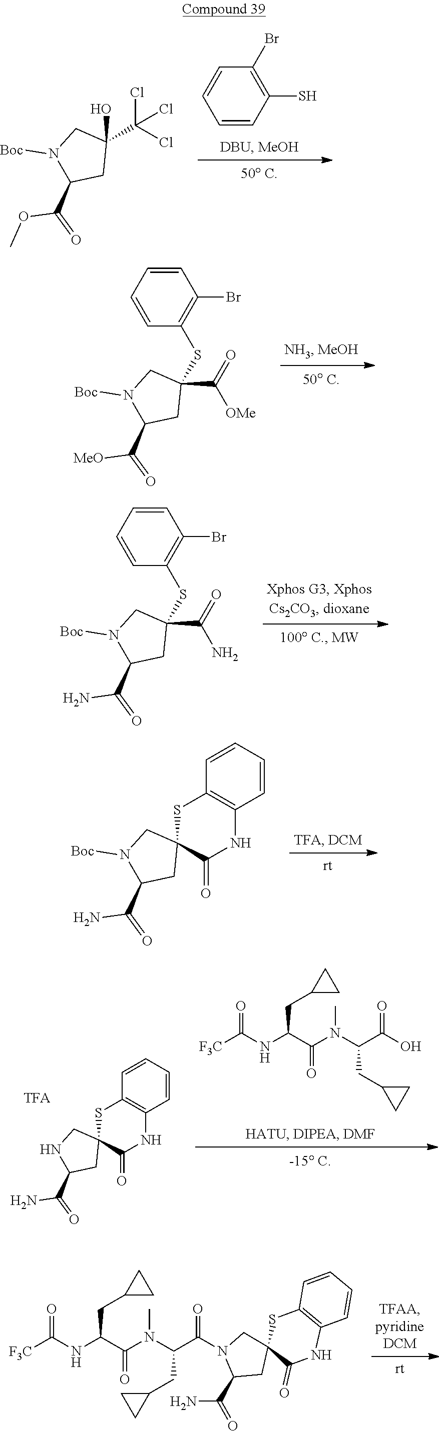

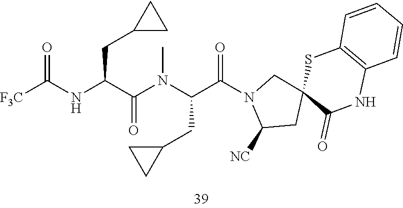 [SMILES: CN(C(=O)[C@H](CC1CC1)NC(=O)C(F)(F)F)[C@@H](CC1CC1)C(=O)N1C[C@@]2(C[C@H]1C#N)Sc1ccccc1NC2=O]

To a mixture of 1-(t-butyl) 2-methyl (2S,4S)-4-hydroxy-4-(trichloromethyl)pyrrolidine-1,2-dicarboxylate (1.0 g, 2.77 mmol) and 2-bromobenzenethiol (0.6 mL, 5.54 mmol) in methanol (10 mL) was added DBU (1.47 g, 9.69 mmol). The mixture was heated at 50° C. for 6 h. The mixture was concentrated under reduced pressure. The residue was purified by flash chromatography on silica gel using a gradient of EA (15 to 20%) in PE to afford 1-(t-butyl) 2,4-dimethyl (2S,4R)-4-((2-bromophenyl)thio)pyrrolidine-1,2,4-tricarboxylate (1.1 g, 84%) as an off-white solid.

1-(t-butyl) 2,4-dimethyl (2S,4R)-4-((2-bromophenyl)thio)pyrrolidine-1,2,4-tricarboxylate (1.4 g; 2.19 mmol.) in NH₃ (7M in methanol, 28 mL) was stirred at 50° C. in a sealed tube for 36 h and then concentrated under reduced pressure to afford t-butyl (2S,4R)-4-((2-bromophenyl)thio)-2,4-dicarbamoylpyrrolidine-1-carboxylate (1.05 g, 81%) as an off-white solid.

A solution of t-butyl (2S,4R)-4-((2-bromophenyl)thio)-2,4-dicarbamoylpyrrolidine-1-carboxylate (950 mg, 2.14 mmol) in dioxane (10 mL) was degassed with nitrogen for 5 min. Xphos (101 mg, 0.214 mmol), Xphos Pd G3 (90 mg, 0.107 mmol, 0.05 eq.) and Cs₂CO₃ (2.08 g, 6.42 mmol) were added. The mixture was heated at 100° C. for 1 h under microwave irradiation. After cooling to rt, the mixture was filtered. The solids were washed with DCM (5 mL). The filtrate was concentrated under reduced pressure. The residue was purified by flash chromatography on silica gel using a gradient of MeOH (2 to 5%) in DCM to afford t-butyl (2R,5'S)-5′-carbamoyl-3-oxo-3,4-dihydrospiro[benzo[b][1,4]thiazine-2,3′-pyrrolidine]-1′-carboxylate (370 mg, 48%) as an off-white solid.

To a solution of t-butyl (2R,5'S)-5′-carbamoyl-3-oxo-3,4-dihydrospiro[benzo[b][1,4]thiazine-2,3′-pyrrolidine]-1′-carboxylate cooled at 0° C. (100 mg, 0.296 mmol) in DCM (1 mL) was added TFA (0.15 mL, 1.48 mmol). The mixture was stirred at rt for 6 h. The mixture was concentrated under reduced pressure and co-evaporated with diethyl ether to afford quantitatively (2R,5'S)-3-oxo-3,4-dihydrospiro[benzo[b][1,4]thiazine-2,3′-pyrrolidine]-5′-carboxamide TFA salt (110 mg, crude) as a colorless oil.

To a solution of (S)-3-cyclopropyl-2-((S)-3-cyclopropyl-N-methyl-2-(2,2,2-trifluoroacetamido)propanamido)propanoic acid (100 mg, 0.285 mmol, 1.0 eq.) and (2R,5'S)-3-oxo-3,4-dihydrospiro[benzo[b][1,4]thiazine-2,3′-pyrrolidine]-5′-carboxamide TFA salt (75 mg, 0.285 mmol) in DMF (1 mL) cooled at −15° C. were added HATU (162 mg, 0.427 mmol) and DIPEA (0.149 mL, 0.855 mmol). The mixture was stirred at −15° C. for 30 min. The mixture was diluted with water (5 mL) and extracted with EA (3×10 mL). The organic phases were combined, washed with water and brine, and dried over Na₂SO₄. The solids were removed by filtration and the filtrate was concentrated under reduced pressure. The residue was purified by flash chromatography on silica gel using a gradient of CH₃OH (3 to 6%) in DCM to afford (2R,5'S)-1′-((S)-3-cyclopropyl-2-((S)-3-cyclopropyl-N-methyl-2-(2,2,2-trifluoroacetamido)propanamido)propanoyl)-3-oxo-3,4-dihydrospiro[benzo[b][1,4]thiazine-2,3′-pyrrolidine]-5′-carboxamide (105 mg, 62%) as an off-white solid.

To a solution of (2R,5'S)-1′-((S)-3-cyclopropyl-2-((S)-3-cyclopropyl-N-methyl-2-(2,2,2-trifluoroacetamido)propanamido)propanoyl)-3-oxo-3,4-dihydrospiro[benzo[b][1,4]thiazine-2,3′-pyrrolidine]-5′-carboxamide (100 mg, 0.167 mmol, 1.0 eq.) in DCM (1 mL) cooled at 0° C. were added pyridine (0.029 mL, 0.367 mmol) and TFAA (0.025 mL, 0.184 mmol). The mixture was stirred at 0° C. for 1 h. The mixture was diluted with water (3 mL) and extracted with DCM (3×10 mL). The organic phases were combined, washed with brine (2×5 mL) and dried over Na₂SO₄. The solids were removed by filtration and the filtrate was concentrated under reduced pressure. The residue was purified by prep-HPLC (Column: UNISIL-C18, 25×150 mm, 8 m; Mobile Phase A: 10 mM NH₄HCO₃ in water, Mobile Phase B: ACN; Flow rate: 22 mL/min; Gradient: 40% to 70% B in 10 min) to afford (S)—N—((S)-1-((2R,5'S)-5′-cyano-3-oxo-3,4-dihydrospiro[benzo[b][1,4]thiazine-2,3′-pyrrolidin]-1′-yl)-3-cyclopropyl-1-oxopropan-2-yl)-3-cyclopropyl-N-methyl-2-(2,2,2-trifluoroacetamido)propanamide (60 mg, 63%) as an off-white solid. ¹H NMR (500 MHz, 363K, DMSO-d₆) δ 10.7 (br. s., 1H), 9.2 (br. s., 1H), 7.29 (m, 1H), 7.25 (m, 1H), 7.08 (m, 1H), 7.02 (m, 1H), 5.16 (m, 1H), 4.91 (m, 1H), 4.80 (m, 1H), 4.21 (m, 1H), 3.74 (m, 1H), 3.01 (s, 3H), 2.64-2.75 (m, 1H), 2.43-2.53 (m, 1H), 1.56-1.74 (m, 4H), 0.71 (m, 1H), 0.61 (m, 1H), 0.33-0.46 (m, 4H), 0.16 (m, 1H), 0.02-0.11 (m, 3H). LC-MS (ESI, m/z): 576 [M−H]⁻.

### Example 40

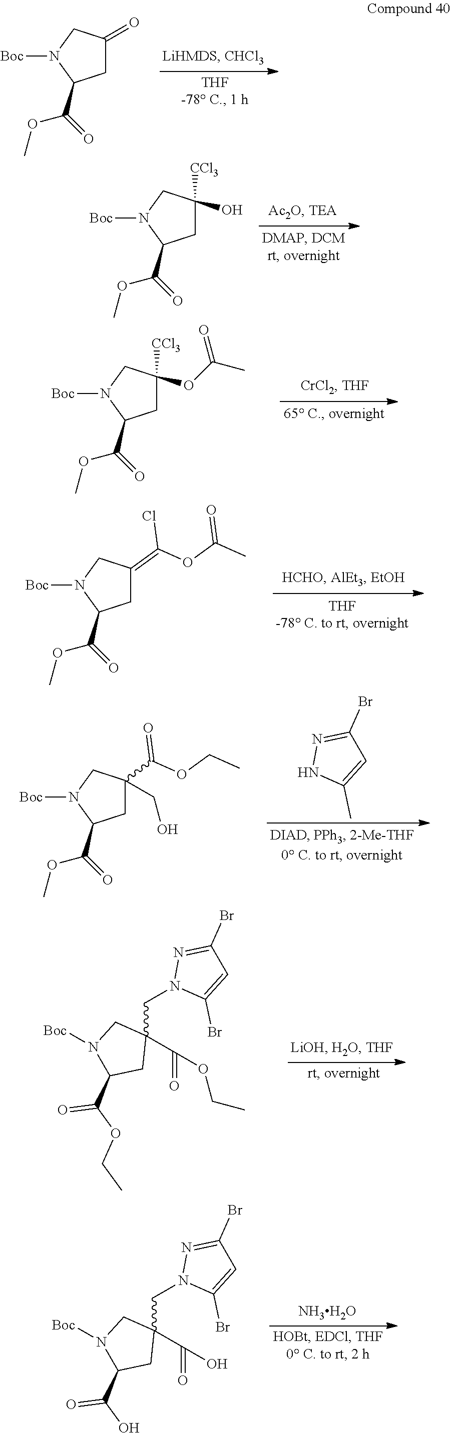

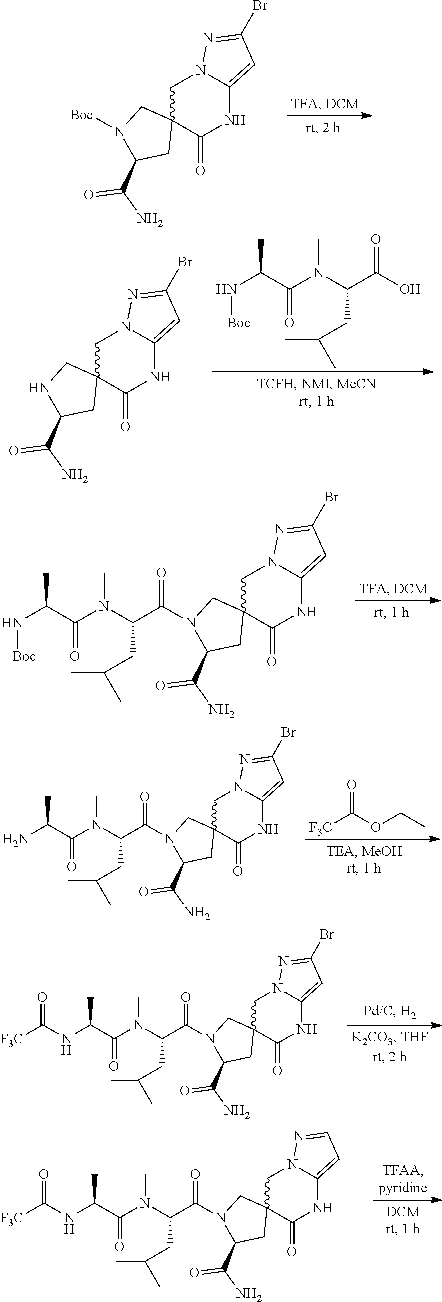

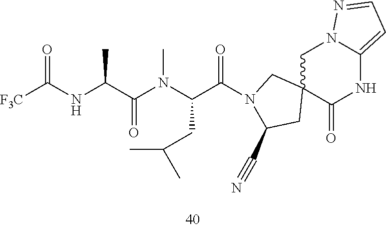 [SMILES: CC(C)C[C@@H](C(=O)N1CC2(C[C@H]1C#N)Cn1nccc1NC2=O)N(C)C(=O)[C@H](C)NC(=O)C(F)(F)F]

To a solution of 1-(t-butyl) 2-methyl (S)-4-oxopyrrolidine-1,2-dicarboxylate (10.0 g×3, 123 mmol) and chloroform (9.8 g×3, 247 mmol) in THF (300 mL) was added lithium bis(trimethylsilyl)amide (82 mL×3, 246 mmol 1M in THF) stirred at −78° C. under nitrogen. The mixture was stirred for 1 h at −78° C. and the reaction was quenched with NH4C1 (sat., aq., 3×150 mL). The mixture was extracted with EA (3×700 mL). The organic layers were combined, washed with brine (2×300 mL) and dried over anhydrous sodium sulfate. The solids were removed by filtration and the filtrate was concentrated under reduced pressure. The crude product was chromatographed on a silica gel column with EA:PE (30%-50%) to provide 1-(t-butyl) 2-methyl (2S,4S)-4-hydroxy-4-(trichloromethyl)pyrrolidine-1,2-dicarboxylate (13 g, crude) as a light yellow solid. ¹H NMR (400 MHz, DMSO-d₆) δ 6.84-6.86 (m, 1H), 4.50-4.71 (m, 1H), 3.74-3.91 (m, 1H), 3.58-3.73 (m, 4H), 2.81-3.16 (m, 1H), 2.51-2.60 (m, 0.3H), 2.13-2.25 (m, 0.63H), 1.30-1.46 (m, 9H). LC-MS (ESI, m/z): 306 [M−56+H]⁺.

To a solution of 1-(t-butyl) 2-methyl (2S,4S)-4-hydroxy-4-(trichloromethyl)pyrrolidine-1,2-dicarboxylate (13.0 g, 35.9 mmol, 1.0 eq.) in DCM (200 mL) were added triethylamine (36.4 g, 358 mmol, 2.5 eq.), acetic anhydride (29.4 g, 287 mmol, 2.0 eq.) and N,N-dimethylpyridin-4-amine (14.1 g, 115 mmol, 0.8 eq.) stirred at rt. The mixture was stirred overnight at rt and the reaction was quenched with water (400 mL). The mixture was extracted with DCM (3×600 mL). The organic layers were combined, washed with brine (2×400 mL) and dried over anhydrous sodium sulfate. The solids were removed by filtration and the filtrate was concentrated under reduced pressure. The crude product was chromatographed on a silica gel column with EA:PE (29%-38%) to provide 1-(t-butyl) 2-methyl (2S,4S)-4-acetoxy-4-(trichloromethyl)pyrrolidine-1,2-dicarboxylate (8.0 g, 55%) as a yellow oil. ¹H NMR (400 MHz, DMSO-d₆) δ 4.65-4.77 (m, 1H), 3.90-4.04 (m, 2H), 3.63-3.71 (m, 3H), 2.97-3.13 (m, 1H), 2.75-2.84 (m, 1H), 2.08-2.11 (m, 3H), 1.35-1.40 (m, 9H). LC-MS (ESI, m/z): 304 [M−boc+H]⁺.

To a solution of chromium (II) chloride (6.05 g, 49.6 mmol) in THF (100 mL) was added 1-(t-butyl) 2-methyl (2S,4S)-4-acetoxy-4-(trichloromethyl)pyrrolidine-1,2-dicarboxylate (8.00 g, 19.8 mmol) stirred under nitrogen at rt. The mixture was stirred overnight at 65° C., then cooled to rt. The reaction was quenched with HCl (10 mL, 0.5M) and then diluted with H₂O (150 mL). The mixture was extracted with EA (3×300 mL). The organic layers were combined, washed with brine (2×200 mL) and dried over anhydrous sodium sulfate. The solids were removed by filtration and the filtrate was concentrated under reduced pressure. The crude product was chromatographed on a silica gel column with EA:PD (20%-25%) to provide 1-(t-butyl) 2-methyl (S,Z)-4-(acetoxychloromethylene)pyrrolidine-1,2-dicarboxylate (2.00 g, 30%) as yellow oil. ¹H NMR (400 MHz, DMSO-d₆) δ 4.39-4.50 (m, 1H), 3.98-4.10 (m, 2H), 3.62-3.71 (m, 3H), 2.91-3.05 (m, 1H), 2.55-2.63 (m, 1H), 2.23 (s, 3H), 1.32-1.47 (m, 9H). LC-MS (ESI, m/z): 234 [M−Boc+H]⁺.

To a solution of anhydrous ethyl alcohol (1.38 g, 30.1 mmol) in anhydrous THF (40 mL) was added triethylaluminum (9.0 mL, 18.0 mmol, 2 M in toluene) stirred at −78° C. under nitrogen. After stirring for 10 mins, 1-(t-butyl) 2-methyl (S,Z)-4-(acetoxychloromethylene)pyrrolidine-1,2-dicarboxylate (2.00 g, 6.01 mmol) and paraformaldehyde (361 mg, 12.0 mmol) were added to the mixture at −78° C. under nitrogen. The mixture was stirred overnight at rt and the reaction was quenched with water (100 mL). The mixture was extracted with EA (3×200 mL). The organic layers were combined, washed with brine (2×100 mL) and dried over anhydrous sodium sulfate. The solids were removed by filtration and the filtrate was concentrated under reduced pressure. The crude product was chromatographed on a silica gel column with EA:PE (40%-50%) to provide 1-(t-butyl) 2,4-diethyl (2S)-4-(hydroxymethyl)pyrrolidine-1,2,4-tricarboxylate (1.0 g, crude) as a light yellow oil. ¹H NMR (400 MHz, DMSO-d₆) δ 5.10-5.25 (m, 1H), 3.99-4.28 (m, 4H), 3.30-3.75 (m, 5H), 2.50-2.56 (m, 0.6H), 2.28-2.36 (m, 0.7H), 1.86-1.90 (m, 0.5H), 1.15-1.40 (m, 15H). LC-MS (ESI, m/z): 246 [M−Boc+H]

To a solution of 1-(t-butyl) 2,4-diethyl (2S)-4-(hydroxymethyl)pyrrolidine-1,2,4-tricarboxylate (1.0 g, 2.90 mmol), triphenylphosphine (2.28 g, 8.70 mmol) and 3,5-dibromo-1H-pyrazole (974 mg, 4.35 mmol) in 2-methyltetrahydrofuran (10 mL) was added diisopropyl azodicarboxylate (1.76 g, 8.70 mmol) at 0° C. under nitrogen. The mixture was stirred overnight at rt and the reaction was quenched with water (100 mL). The mixture was extracted with EA (3×200 mL). The organic layers were combined, washed with brine (2×100 mL) and dried over anhydrous sodium sulfate. The solids were removed by filtration and the filtrate was concentrated under reduced pressure. The crude product was chromatographed on a silica gel column with EA:PE (19%-25%) to provide 1-(t-butyl) 2,4-diethyl (2S)-4-((3,5-dibromo-1H-pyrazol-1-yl)methyl)pyrrolidine-1,2,4-tricarboxylate (1.1 g, crude) as a light yellow semi-solid. ¹H NMR (400 MHz, DMSO-d₆) δ 6.69 (s, 1H), 4.29-4.59 (m, 2H), 3.96-4.23 (m, 4H), 3.73-3.94 (m, 1H), 3.56-3.70 (m, 1H), 3.40-3.53 (m, 1H), 2.62-2.73 (m, 1H), 2.03-2.31 (m, 1H), 1.29-1.44 (m, 9H), 1.11-1.26 (m, 6H). LC-MS (ESI, m/z): 452 [M-Boc+H]⁺.

To a mixture of 1-(t-butyl) 2,4-diethyl (2S)-4-((3,5-dibromo-1H-pyrazol-1-yl)methyl)pyrrolidine-1,2,4-tricarboxylate (1.10 g, 1.99 mmol) in THF (10 mL) was added LiOH (575 mg, 23.9 mmol, in 10 mL H₂O) at rt. The mixture was stirred overnight at rt and then diluted with H₂O (50 mL). The mixture was extracted with EA (2×100 mL). The aqueous layers were combined and was acidified to pH=4 with HCl (1 M). The mixture was extracted with EA (4×100 mL). The organic layers were combined and dried over anhydrous sodium sulfate. The solids were removed by filtration and the filtrate was concentrated under reduced pressure to provide (2S)-1-(t-butoxycarbonyl)-4-((3,5-dibromo-1H-pyrazol-1-yl)methyl)pyrrolidine-2,4-dicarboxylic acid (900 mg, crude) as a light yellow solid. ¹H NMR (400 MHz, DMSO-d₆) δ 12.69 (br, 2H), 6.68-6.70 (m, 1H), 4.36-4.49 (m, 2H), 3.86-4.16 (m, 2H), 3.39-3.66 (m, 1H), 2.65-2.71 (m, 0.6H), 2.37-2.47 (m, 0.5H), 2.15-2.32 (m, 0.6H), 1.93-2.02 (m, 0.5H), 1.31-1.45 (m, 9H). LC-MS (ESI, m/z): 440 [M-56+H]⁺.

To a solution of (2S)-1-(t-butoxycarbonyl)-4-((3,5-dibromo-1H-pyrazol-1-yl)methyl)pyrrolidine-2,4-dicarboxylic acid (900 mg, 1.82 mmol) in THF (18 mL) were added 1-hydroxybenzotriazole (1.48 g, 10.9 mmol) and 1-(3-dimethylaminopropyl)-3-ethylcarbodiimide HCl (1.74 g, 9.10 mmol). The mixture was stirred at 0° C. After stirred for 1 h, NH₄OH (21 mL) was added. The mixture was stirred for 1 h at rt and then diluted with water (80 mL). The mixture was extracted with EA (3×150 mL). The organic layers were combined, washed with brine (2×80 mL) and dried over anhydrous sodium sulfate. The solids were removed by filtration and the filtrate was concentrated under reduced pressure. The crude product was purified by TLC (Mobile phase: MeOH/DCM=1:10; Rf=0.4; detection: UV) to provide t-butyl (2S)-2,4-dicarbamoyl-4-((3,5-dibromo-1H-pyrazol-1-yl)methyl)pyrrolidine-1-carboxylate (730 mg, crude) as a yellow solid. ¹H NMR (400 MHz, DMSO-d₆) δ 7.46-7.63 (m, 1H), 7.27-7.44 (m, 1H), 7.14-7.21 (m, 1H), 6.90-6.99 (m, 1H), 6.59-6.68 (m, 1H), 4.24-4.52 (m, 2H), 3.87-4.13 (m, 2H), 3.42-3.61 (m, 1H), 2.60-2.69 (m, 0.6H), 2.42-2.48 (m, 0.6H), 2.01-2.06 (m, 0.5H), 1.66-1.72 (m, 0.7H), 1.26-1.44 (m, 9H). LC-MS (ESI, m/z): 394 [M-Boc+H]⁺.

To a solution of t-butyl (2S)-2,4-dicarbamoyl-4-((3,5-dibromo-1H-pyrazol-1-yl)methyl)pyrrolidine-1-carboxylate (700 mg, 1.42 mmol) in THF (6 mL) were added cuprous iodide (189 mg, 0.994 mmol), cesium carbonate (925 mg, 2.84 mmol) and N,N′-dimethyl-1,2-ethanediamine (200 mg, 2.27 mmol) stirred under nitrogen at rt. The mixture was stirred overnight at 70° C. The mixture was filtered through a celite pad, and washed with DCM (3×100 mL) and CH₃OH (3×50 mL). The filtrate was concentrated under reduced pressure. The crude product was purified by TLC (Mobile phase: CH₃OH:DCM=1:10; Rf=0.3; detection: UV) to provide t-butyl (5'S)-2-bromo-5′-carbamoyl-5-oxo-4,5-dihydro-7H-spiro[pyrazolo[1,5-a]pyrimidine-6,3′-pyrrolidine]-1′-carboxylate (140 mg, 23%) as a yellow solid. ¹H NMR (400 MHz, DMSO-d₆) δ 11.11-11.16 (m, 1H), 7.27-7.50 (m, 1H), 6.89-7.08 (m, 1H), 5.67-5.71 (m, 1H), 4.09-4.42 (m, 3H), 3.38-3.82 (m, 2H), 2.52-2.56 (m, 0.7H), 2.01-2.26 (m, 1H), 1.68-2.21 (m, 0.6H), 1.26-1.49 (m, 9H). LC-MS (ESI, m/z): 358 [M-56+H]⁺.

To a solution of t-butyl (5'S)-2-bromo-5′-carbamoyl-5-oxo-4,5-dihydro-7H-spiro[pyrazolo[1,5-a]pyrimidine-6,3′-pyrrolidine]-1′-carboxylate (140 mg, 0.339 mmol) in DCM (1.5 mL) was added TFA (0.5 mL). The mixture was stirred for 1 h at rt and concentrated under reduced pressure to afford (5'S)-2-bromo-5-oxo-4,5-dihydro-7H-spiro[pyrazolo[1,5-a]pyrimidine-6,3′-pyrrolidine]-5′-carboxamide (110 mg, crude) as a brown oil. LC-MS (ESI, m/z): 314 [M+H]⁺.

To a solution of (5'S)-2-bromo-5-oxo-4,5-dihydro-7H-spiro[pyrazolo[1,5-a]pyrimidine-6,3′-pyrrolidine]-5′-carboxamide (110 mg, 0.350 mmol), (2S)-2-[(2S)-2-[(t-butoxycarbonyl)amino]-N-methylpropanamido]-4-methylpentanoic acid (111 mg, 0.350 mmol) and N,N,N,N-tetramethylchloroformamidinium hexafluorophosphate (128 mg, 0.455 mmol) in ACN (2 mL) was added N-methylmorpholine (288 mg, 3.50 mmol) stirred at 0° C. The mixture was stirred for 1 h at rt and the reaction was quenched with water (20 mL). The mixture was extracted with EA (3×80 mL). The organic layers were combined, washed with brine (2×30 mL) and dried over anhydrous sodium sulfate. The solids were removed by filtration and the filtrate was concentrated under reduced pressure. The crude product was purified by TLC (Mobile phase: MeOH:DCM=1:12; Rf=0.4; detection: UV) to provide t-butyl ((2S)-1-(((2S)-1-((5'S)-2-bromo-5′-carbamoyl-5-oxo-4,5-dihydro-7H-spiro[pyrazolo[1,5-a]pyrimidine-6,3′-pyrrolidin]-1′-yl)-4-methyl-1-oxopentan-2-yl)(methyl)amino)-1-oxopropan-2-yl)carbamate (160 mg, 74%) as a yellow solid. LC-MS (ESI, m/z): 634 [M+Na]⁺.

To a solution of t-butyl ((2S)-1-(((2S)-1-((5'S)-2-bromo-5′-carbamoyl-5-oxo-4,5-dihydro-7H-spiro[pyrazolo[1,5-a]pyrimidine-6,3′-pyrrolidin]-1′-yl)-4-methyl-1-oxopentan-2-yl)(methyl)amino)-1-oxopropan-2-yl)carbamate (150 mg, 0.245 mmol, 1.0 eq.) in DCM (1.5 mL) was added TFA (0.5 mL). The mixture was stirred for 1 h at rt and concentrated under reduced pressure to afford (5'S)-1′-(N-(L-alanyl)-N-methyl-L-leucyl)-2-bromo-5-oxo-4,5-dihydro-7H-spiro[pyrazolo[1,5-a]pyrimidine-6,3′-pyrrolidine]-5′-carboxamide (130 mg, crude) as a brown oil. LC-MS (ESI, m/z): 512 [M+H]⁺.

To a mixture of (5'S)-1′-(N-(L-alanyl)-N-methyl-L-leucyl)-2-bromo-5-oxo-4,5-dihydro-7H-spiro[pyrazolo[1,5-a]pyrimidine-6,3′-pyrrolidine]-5′-carboxamide (130 mg, 0.254 mmol) in MeOH (1.5 mL) were added triethylamine (308 mg, 3.05 mmol) and ethyl 2,2,2-trifluoroacetate (360 mg, 2.54 mmol). The mixture was stirred for 1 h at rt and the reaction was quenched with water (10 mL). The mixture was concentrated under reduced pressure to remove MeOH and acidified to pH=5 with HCl (1 M). The mixture was extracted with DCM (3×80 mL). The organic layers were combined, washed with brine (2×50 mL) and dried over anhydrous sodium sulfate. The solids were removed by filtration and the filtrate was concentrated under reduced pressure. The crude product was purified by TLC (Mobile phase: CH₃OH:DCM=1:10; Rf=0.4; detection: UV) to provide (5'S)-2-bromo-1′-(N-methyl-N-((2,2,2-trifluoroacetyl)-L-alanyl)-L-leucyl)-5-oxo-4,5-dihydro-7H-spiro[pyrazolo[1,5-a]pyrimidine-6,3′-pyrrolidine]-5′-carboxamide (110 mg, 71%) as a light yellow solid. LC-MS (ESI, m/z): 630 [M+Na]⁺.

To a mixture of (5'S)-2-bromo-1′—(N-methyl-N-((2,2,2-trifluoroacetyl)-L-alanyl)-L-leucyl)-5-oxo-4,5-dihydro-7H-spiro[pyrazolo[1,5-a]pyrimidine-6,3′-pyrrolidine]-5′-carboxamide (60.0 mg, 0.099 mmol) and potassium carbonate (20.4 mg, 0.149 mmol) in THF (2 mL) was added 10% Pd on activated carbon (30 mg) under hydrogen. The mixture was stirred for 2 h at rt under hydrogen. The mixture was filtered through a celite pad and washed with DCM (50 mL) and MeOH (50 mL). The filtrate was concentrated under reduced pressure to afford (5'S)-1′-(N-methyl-N-((2,2,2-trifluoroacetyl)-L-alanyl)-L-leucyl)-5-oxo-4,5-dihydro-7H-spiro[pyrazolo[1,5-a]pyrimidine-6,3′-pyrrolidine]-5′-carboxamide (40 mg, crude) as a yellow solid. LC-MS (ESI, m/z): 530 [M+H]⁺.

To a solution of (5'S)-1′-(N-methyl-N-((2,2,2-trifluoroacetyl)-L-alanyl)-L-leucyl)-5-oxo-4,5-dihydro-7H-spiro[pyrazolo[1,5-a]pyrimidine-6,3′-pyrrolidine]-5′-carboxamide (45.0 mg, 0.085 mmol) in DCM (1 mL) were added pyridine (33.6 mg, 0.425 mmol) and 2,2,2-trifluoroacetic anhydride (32.1 mg, 0.153 mmol). The mixture was stirred 1 h at rt and the reaction was quenched with water (30 mL). The mixture was extracted with DCM (3×50 mL). The organic layers were combined, washed with brine (2×30 mL) and dried over anhydrous sodium sulfate. The solids were removed by filtration and the filtrate was concentrated under reduced pressure. The crude product was purified by prep-HPLC (Column: Xselect CSH Prep OBD C18 Column, 30×150 mm, 5 m; Mobile Phase A: Water (0.1% FA), Mobile Phase B: ACN; Flow rate: 60 mL/min; Gradient: 30% B to 50% B in 9 min; Wave Length: 254 nm/220 nm) to provide (2S)—N-((2S)-1-((5'S)-5′-cyano-5-oxo-4,5-dihydro-7H-spiro[pyrazolo[1,5-a]pyrimidine-6,3′-pyrrolidin]-1′-yl)-4-methyl-1-oxopentan-2-yl)-N-methyl-2-(2,2,2-trifluoroacetamido)propanamide (8.7 mg, 19%) as a white solid. ¹H NMR (400 MHz, 80° C., DMSO-d₆) δ 9.72-10.8 (m, 1H), 7.20-7.31 (m, 1H), 5.54-5.61 (m, 1H), 5.02-5.22 (m, 1H), 4.62-4.93 (m, 2H), 4.04-4.33 (m, 2H), 3.35-3.90 (m, 2H), 2.86-2.96 (m, 3H), 2.54-2.72 (m, 1H), 2.10-2.29 (m, 1H), 1.30-1.70 (m, 3H), 1.01-1.29 (m, 3H), 0.72-0.91 (m, 6H). LC-MS (ESI, m/z): 512 [M+H]⁺.

### Example 41

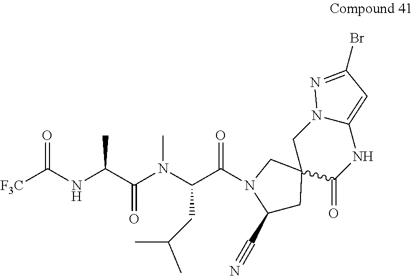 [SMILES: CC(C)C[C@@H](C(=O)N1CC2(C[C@H]1C#N)Cn1nc(Br)cc1NC2=O)N(C)C(=O)[C@H](C)NC(=O)C(F)(F)F]

To a solution of (5'S)-2-bromo-1′-(N-methyl-N-((2,2,2-trifluoroacetyl)-L-alanyl)-L-leucyl)-5-oxo-4,5-dihydro-7H-spiro[pyrazolo[1,5-a]pyrimidine-6,3′-pyrrolidine]-5′-carboxamide (35.0 mg, 0.058 mmol) in DCM (1 mL) were added pyridine (22.8 mg, 0.290 mmol) and 2,2,2-trifluoroacetic anhydride (21.8 mg, 0.104 mmol). The mixture was stirred 1 h at rt and the reaction was quenched with water (30 mL). The mixture was extracted with DCM (3×50 mL). The organic layers were combined, washed with brine (2×30 mL) and dried over anhydrous sodium sulfate. The solids were removed by filtration and the filtrate was concentrated under reduced pressure. The crude product was purified by prep-HPLC (Column: XBridge Prep OBD C18 Column, 19×250 mm, 5 m; Mobile Phase A: Water (0.1% FA), Mobile Phase B: ACN; Flow rate: 25 mL/min; Gradient: 21 to 51% B in 15 min; Wave Length: 254 nm/220 nm) to provide (2S)—N-((2S)-1-((5'S)-2-bromo-5′-cyano-5-oxo-4,5-dihydro-7H-spiro[pyrazolo[1,5-a]pyrimidine-6,3′-pyrrolidin]-1′-yl)-4-methyl-1-oxopentan-2-yl)-N-methyl-2-(2,2,2-trifluoroacetamido)propanamide (6.3 mg, 18%) as a white solid. ¹H NMR (400 MHz, 80° C., DMSO-d₆) δ 9.30-9.40 (m, 1H), 5.70-5.74 (m, 1H), 5.11-5.22 (m, 1H), 4.90-5.05 (m, 1H), 4.69-4.80 (m, 1H), 4.15-4.36 (m, 2H), 3.84-3.89 (m, 1H), 3.66-3.70 (m, 1H), 3.51-3.59 (m, 1H), 2.83-2.94 (m, 3H), 2.62-2.66 (m, 1H), 2.31-2.37 (m, 1H), 1.61-1.78 (m, 3H), 1.20-1.59 (m, 3H), 0.82-0.91 (m, 6H). LC-MS (ESI, m/z): 590 [M+H]⁺.

### Example 42

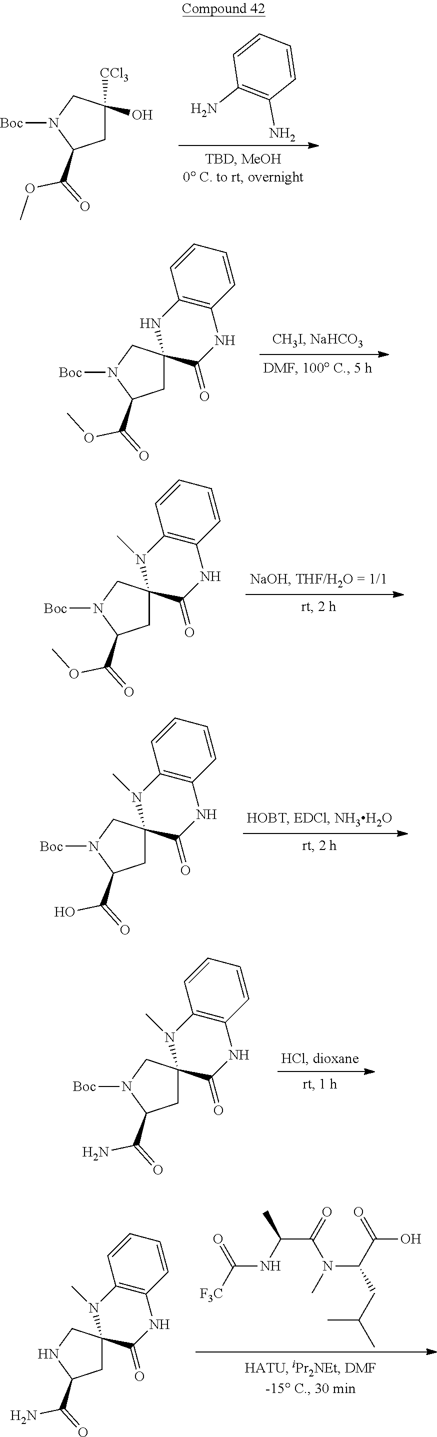

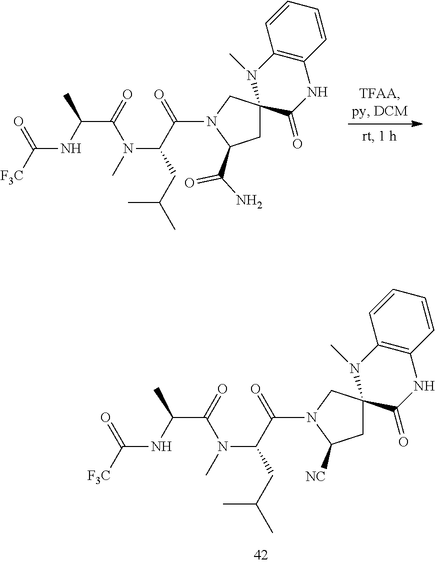 [SMILES: CC(C)C[C@@H](C(=O)N1C[C@]2(C[C@H]1C(N)=O)C(=O)Nc1ccccc1N2C)N(C)C(=O)[C@H](C)NC(=O)C(F)(F)F ; CC(C)C[C@@H](C(=O)N1C[C@]2(C[C@H]1C#N)C(=O)Nc1ccccc1N2C)N(C)C(=O)[C@H](C)NC(=O)C(F)(F)F]

To a mixture of 1-(t-butyl) 2-methyl (2S,4S)-4-hydroxy-4-(trichloromethyl)pyrrolidine-1,2-dicarboxylate (2.0 g, 5.52 mmol) and benzene-1,2-diamine (1.79 g, 16.6 mmol) in MeOH (20 mL) was added 1,5,7-Triazabicyclo[4.4.0]dec-5-enee (2.69 g, 19.3 mmol, 3.50 eq.) at 0° C. The mixture was stirred overnight at rt. The mixture was purified by C18 column with CH₃CN/Water (0.05% TFA). The fraction was concentrated under reduced pressure. The crude product was chromatographed on a silica gel column with EA:PE (3:7) to provide 1-(t-butyl) 5-methyl (3R,5S)-3′-oxo-3′,4′-dihydro-1′H-spiro[pyrrolidine-3,2′-quinoxaline]-1,5-dicarboxylate (950 mg, 43%) as a yellow solid. ¹H NMR (400 MHz, DMSO-d₆) δ 10.55 (s, 1H), 6.52-6.92 (m, 5H), 4.41-4.59 (m, 1H), 3.60-3.77 (m, 3H), 3.37-3.55 (m, 2H), 2.14-2.45 (m, 2H), 1.28-1.44 (m, 9H). LC-MS (ESI, m/z): 306 [M−56+H]⁺.

To a mixture of 1-(t-butyl) 5-methyl (3R,5S)-3′-oxo-3′,4′-dihydro-1′H-spiro[pyrrolidine-3,2′-quinoxaline]-1,5-dicarboxylate (940 mg, 2.60 mmol) in DMF (47 mL) were added iodomethane (3.70 g, 26.0 mmol) and sodium bicarbonate (437 mg, 5.20 mmol, 2.0 eq.). The mixture was stirred for 5 h at 100° C. The mixture was filtered and the filter cake was washed with EA (2×30 mL). The filtrate was concentrated under reduced pressure and water (50 mL) was added. The mixture was extracted with EA (3×100 mL). The organic layers were combined, washed with brine (2×50 mL) and dried over anhydrous sodium sulfate. The solids were removed by filtration and the filtrate was concentrated under reduced pressure. The crude product was chromatographed on a silica gel column with EA:PE (2:3) to provide 1-(t-butyl) 5-methyl (3R,5S)-1′-methyl-3′-oxo-3′,4′-dihydro-1′H-spiro[pyrrolidine-3,2′-quinoxaline]-1,5-dicarboxylate (650 mg, 66%) as a yellow oil. ¹H NMR (400 MHz, DMSO-d₆) δ 10.67-10.84 (m, 1H), 6.75-7.06 (m, 4H), 4.38-4.60 (m, 1H), 3.54-3.71 (m, 5H), 2.70-2.84 (m, 3H), 2.53-2.62 (m, 2H), 1.28-1.44 (m, 9H). LC-MS (ESI, m/z): 773 [2M+Na]⁺.

To a stirred mixture of 1-(t-butyl) 5-methyl (3R,5S)-1′-methyl-3′-oxo-3′,4‘-dihydro-1’H-spiro[pyrrolidine-3,2′-quinoxaline]-1,5-dicarboxylate (550 mg, 1.47 mmol) in THF (17 mL) and water (17 mL) was added NaOH (293 mg, 7.33 mmol) at rt. The mixture was stirred for 2 h and acidified to pH=3 with HCl (2M). The mixture was extracted with EA (3×100 mL). The organic layers were combined and dried over anhydrous sodium sulfate. The organic layers were concentrated under reduced pressure to afford (3R,5S)-1-(t-butoxycarbonyl)-1′-methyl-3′-oxo-3′,4′-dihydro-1′H-spiro[pyrrolidine-3,2′-quinoxaline]-5-carboxylic acid (460 mg, crude) as a yellow solid. LC-MS (ESI, m/z): 362 [M+H]⁺.

To a mixture of (3R,5S)-1-(t-butoxycarbonyl)-1′-methyl-3′-oxo-3′,4′-dihydro-1′H-spiro[pyrrolidine-3,2′-quinoxaline]-5-carboxylic acid (460 mg, 1.27 mmol) in THF (15 mL) were added 1-hydroxybenzotriazole (430 mg, 3.18 mmol) and 1-(3-dimethylaminopropyl)-3-ethylcarbodiimide HCl (488 mg, 2.54 mmol). The mixture was stirred for 30 mins at 0° C. and then ammonia (5 mL, 30% in water) was added at 0° C. The mixture was stirred for 2 h at rt and then diluted with water (15 mL). The mixture was extracted with EA (3×80 mL). The organic layers were combined, washed with brine (2×15 mL) and dried over magnesium sulfate anhydrous. The solids were removed by filtration and the filtrate was concentrated under reduced pressure. The crude product was purified by C18 column with CH₃CN/Water (0.05% TFA). The fraction was concentrated under reduced pressure to provide t-butyl (3R,5S)-5-carbamoyl-1′-methyl-3′-oxo-3′,4′-dihydro-1′H-spiro[pyrrolidine-3,2′-quinoxaline]-1-carboxylate (360 mg, 78%) as a yellow solid. ¹H NMR (400 MHz, DMSO-d₆) δ 10.71 (s, 1H), 7.21-7.50 (m, 1H), 6.22-7.02 (m, 5H), 4.20-4.31 (m, 1H), 3.65-4.75 (m, 2H), 2.67-2.87 (m, 3H), 2.21-2.46 (m, 2H), 1.24-1.49 (m, 9H). LC-MS (ESI, m/z): 361 [M+H]⁺.

A mixture of t-butyl (3R,5S)-5-carbamoyl-1′-methyl-3′-oxo-3′,4′-dihydro-1′H-spiro[pyrrolidine-3,2′-quinoxaline]-1-carboxylate (150 mg, 0.417 mmol) in HCl (2 mL, 4 M in 1,4-dioxane) was stirred for 1 h at rt. The mixture was concentrated under reduced pressure to afford (3R,5S)-1′-methyl-3′-oxo-3′,4′-dihydro-1′H-spiro[pyrrolidine-3,2′-quinoxaline]-5-carboxamide HCl (150 mg, crude). LC-MS (ESI, m/z): 261 [M+H]⁺.

To a stirred mixture of (3R,5S)-1′-methyl-3′-oxo-3′,4′-dihydro-1′H-spiro[pyrrolidine-3,2′-quinoxaline]-5-carboxamide HCl (150 mg, 0.576 mmol), N-methyl-N-((2,2,2-trifluoroacetyl)-L-alanyl)-L-leucine (186 mg, 0.593 mmol) and HATU (263 mg, 0.691 mmol) in DMF (2 mL) was added N-ethyl-N-isopropylpropan-2-amine (149 mg, 1.15 mmol). The mixture was stirred for 30 mins at −15° C. and the reaction was quenched with water (10 mL). The mixture was extracted with EA (3×50 mL). The organic layers were combined, washed with brine (2×15 mL) and dried over anhydrous sodium sulfate. The solids were removed by filtration and the filtrate was concentrated under reduced pressure to afford the crude product, which was purified by TLC (Mobile phase: MeOH/DCM=1:9; Rf=0.4; detection: UV) to provide (3R,5S)-1′-methyl-1-(N-methyl-N-((2,2,2-trifluoroacetyl)-L-alanyl)-L-leucyl)-3′-oxo-3′,4′-dihydro-1′H-spiro[pyrrolidine-3,2′-quinoxaline]-5-carboxamide (240 mg, 75%) as a white solid. ¹H NMR (400 MHz, DMSO-d₆) δ 10.80 (s, 1H), 9.70 (br, 1H), 7.25-7.45 (m, 1H), 6.81-7.00 (m, 5H), 5.15-5.39 (m, 1H), 4.64-5.73 (m, 1H), 4.37-4.49 (m, 1H), 4.05-4.32 (m, 1H), 3.55-4.03 (m, 1H), 2.84-3.15 (m, 3H), 2.69-2.82 (m, 3H), 2.05-2.47 (m, 2H), 1.30-1.68 (m, 5H), 1.00-1.15 (m, 1H), 0.72-0.93 (m, 6H). LC-MS (ESI, m/z): 555 [M+H]⁺.

To a mixture of (3R,5S)-1′-methyl-1-(N-methyl-N-((2,2,2-trifluoroacetyl)-L-alanyl)-L-leucyl)-3′-oxo-3′,4′-dihydro-1′H-spiro[pyrrolidine-3,2′-quinoxaline]-5-carboxamide (240 mg, 0.433 mmol) in DCM (10 mL) were added pyridine (171 mg, 2.17 mmol) and trifluoroacetic anhydride (136 mg, 0.650 mmol). The mixture was stirred for 1 h at rt and the reaction was quenched with water (10 mL). The mixture was extracted with DCM (3×30 mL). The organic layers were combined, washed with brine (2×10 mL) and dried over anhydrous sodium sulfate. The solids were removed by filtration and the filtrate was concentrated under reduced pressure to afford the crude product that was purified by prep-HPLC (Column: Xselect CSH Prep OBD C18 Column 30×150 mm, 5 μm; Mobile Phase A: Water (0.1% FA), Mobile Phase B: ACN; Flow rate: 60 mL/min; Gradient: 42% B to 50% B in 8 min; Wave Length: 220 nm) to provide (S)—N—((S)-1-((3R,5S)-5-cyano-1′-methyl-3′-oxo-3′,4′-dihydro-1′H-spiro[pyrrolidine-3,2′-quinoxalin]-1-yl)-4-methyl-1-oxopentan-2-yl)-N-methyl-2-(2,2,2-trifluoroacetamido) propanamide (79.3 mg, 34%) as a white solid. ¹H NMR (400 MHz, 60° C., DMSO-d₆) δ 10.73 (br, 1H), 9.52 (br, 1H), 6.35-7.31 (m, 4H), 4.91-5.82 (m, 2H), 4.71 (br, 1H), 3.63-4.51 (m, 2H), 3.03-3.60 (m, 1H), 2.92-3.02 (m, 2H), 2.59-2.81 (m, 5H), 1.33-2.07 (m, 3H), 1.11-1.32 (m, 3H), 0.40-1.10 (m, 6H). LC-MS (ESI, m/z): 535 [M−H]⁻.

### Example 43

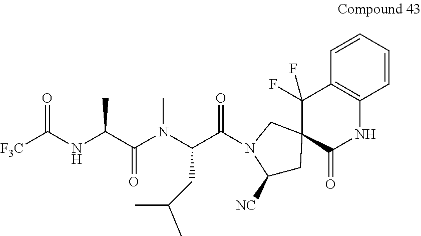 [SMILES: CC(C)C[C@@H](C(=O)N1C[C@]2(C[C@H]1C#N)C(=O)Nc1ccccc1C2(F)F)N(C)C(=O)[C@H](C)NC(=O)C(F)(F)F]

To a mixture of Zn power (1.43 g, 21.8 mmol) in THF (20 mL) was added chlorotrimethylsilane (355 mg, 3.29 mmol) at rt under nitrogen. After stirring for 0.5 h, 1-(t-butyl) 2,4-dimethyl (2S,4R)-4-bromopyrrolidine-1,2,4-tricarboxylate (4.00 g, 10.9 mmol, in 15 mL THF) and 2-bromobenzaldehyde (4.03 g, 21.8 mmol) were added. The mixture was stirred overnight at rt and the reaction was quenched with aqueous ammonium chloride (100 mL). The mixture was extracted with EA (3×200 mL). The organic layers were combined, washed with brine (2×100 mL) and dried over anhydrous sodium sulfate. The solids were removed by filtration and the filtrate was concentrated under reduced pressure. The crude product was chromatographed on a silica gel column with EA:PE (40-55%), and then by a C18 column with CH₃CN/Water (0.05% TFA). The fraction was concentrated under reduced pressure to provide 1-(t-butyl) 2,4-dimethyl (2S,4S)-4-((2-bromophenyl)(hydroxy)methyl)pyrrolidine-1,2,4-tricarboxylate (2.4 g, 46%) as a semi-solid.

¹H NMR (400 MHz, DMSO-d₆) δ 7.22-7.66 (m, 4H), 5.76 (br, 1H), 5.27-5.76 (m, 1H), 3.71-4.06 (m, 3H), 3.54-3.62 (m, 6H), 2.51-2.64 (m, 1H), 2.21-2.50 (m, 1H), 1.25-1.41 (m, 9H). LC-MS (ESI, m/z): 372 [M−Boc+H]⁺.

To a solution of pyridine (2.41 g, 30.5 mmol) in DCM (30 mL) was added chromium trioxide (1.52 g, 15.2 mmol). After stirring for 1 h, 1-(t-butyl) 2,4-dimethyl (2S,4S)-4-((2-bromophenyl)(hydroxy)methyl)pyrrolidine-1,2,4-tricarboxylate (2.40 g, 5.08 mmol) was added. The mixture was stirred overnight at rt and the reaction was quenched with water (100 mL). The mixture was extracted with DCM (3×200 mL). The organic layers were combined, washed with brine (2×100 mL) and dried over anhydrous sodium sulfate. The solids were removed by filtration and the filtrate was concentrated under reduced pressure. The crude product was chromatographed on a silica gel column with MeOH:DCM (6%) to provide 1-(t-butyl) 2,4-dimethyl (2S,4S)-4-(2-bromobenzoyl)pyrrolidine-1,2,4-tricarboxylate (800 mg, 33%) as a light yellow solid. ¹H NMR (400 MHz, DMSO-d₆) δ 7.74-7.77 (m, 1H), 7.37-7.53 (m, 3H), 4.20-4.33 (m, 1H), 3.68-4.04 (m, 2H), 3.63-3.65 (m, 6H), 2.95-2.98 (m, 1H), 2.51-2.63 (m, 1H), 1.24-1.42 (m, 9H). LC-MS (ESI, m/z): 370 [M−Boc+H]⁺.

To a solution of 1-(t-butyl) 2,4-dimethyl (2S,4S)-4-(2-bromobenzoyl)pyrrolidine-1,2,4-tricarboxylate (800 mg, 1.70 mmol) in diethylaminosulfur trifluoride (5 mL) was added EtOH (31.4 mg, 0.680 mmol) stirred at 0° C. The mixture was stirred overnight at 45° C. and then diluted with DCM (50 mL) at 0° C. The reaction was quenched with ice saturated aqueous sodium bicarbonate (50 mL). The mixture was extracted with DCM (3×100 mL). The organic layers were combined, washed with brine (2×100 mL) and dried over anhydrous sodium sulfate. The solids were removed by filtration and the filtrate was concentrated under reduced pressure to afford the crude product, which was purified by C18 column with CH₃CN/Water (0.05% TFA). The fraction was concentrated under reduced pressure to afford 1-(t-butyl) 2,4-dimethyl (2S,4R)-4-((2-bromophenyl)difluoromethyl)pyrrolidine-1,2,4-tricarboxylate (340 mg, 40%). ¹H NMR (400 MHz, DMSO-d₆) δ 7.76-7.79 (m, 1H), 7.44-7.59 (m, 3H), 4.25-4.29 (m, 1H), 3.98-4.20 (m, 1H), 3.70-3.80 (m, 1H), 3.55-3.69 (m, 6H), 2.80-2.91 (m, 1H), 2.51-2.55 (m, 1H), 1.24-1.46 (m, 9H). LC-MS (ESI, m/z): 392 [M−Boc+H]⁺.

To a mixture of 1-(t-butyl) 2,4-dimethyl (2S,4R)-4-((2-bromophenyl)difluoromethyl)pyrrolidine-1,2,4-tricarboxylate (300 mg, 0.609 mmol, 1.0 eq.) in THF (3 mL) was added NaOH (122 mg, 3.05 mmol, in 3 mL H₂O) at rt. The mixture was stirred overnight at rt and then diluted with H₂O (30 mL). The mixture was extracted with EA (2×100 mL). The aqueous layers were combined and acidified to pH=4 with HCl (1 M). The mixture was extracted with EA (4×100 mL). The organic layers were combined and dried over anhydrous sodium sulfate. The solids were removed by filtration and the filtrate was concentrated under reduced pressure to provide (2S,4R)-4-((2-bromophenyl)difluoromethyl)-1-(t-butoxycarbonyl)pyrrolidine-2,4-dicarboxylic acid (250 mg, crude) as a light yellow solid. LC-MS (ESI, m/z): 408 [M-56+H]⁺.

To a solution of (2S,4R)-4-((2-bromophenyl)difluoromethyl)-1-(t-butoxycarbonyl)pyrrolidine-2,4-dicarboxylic acid (240 mg, 0.516 mmol) in THF (1 mL) were added 1-hydroxybenzotriazole (419 mg, 3.10 mmol) and 1-(3-dimethylaminopropyl)-3-ethylcarbodiimide hydrochloride (496 mg, 2.58 mmol) stirred at 0° C. After stirring for 1 h, NH₄OH (2.4 mL) was added. The mixture was stirred for 1 h at rt and then diluted with water (50 mL). The mixture was extracted with EA (3×100 mL). The organic layers were combined and dried over anhydrous sodium sulfate. The solids were removed by filtration and the filtrate was concentrated under reduced pressure. The crude product was purified by C18 column with CH₃CN/Water (0.05% NH₄HCO₃, pH-10), (25%). The fraction was concentrated under reduced pressure to provide t-butyl (2S,4R)-4-((2-bromophenyl)difluoromethyl)-2,4-dicarbamoylpyrrolidine-1-carboxylate (160 mg, crude) as a yellow solid. ¹H NMR (400 MHz, DMSO-d₆) δ 7.67-7.81 (m, 1H), 7.40-7.56 (m, 5H), 7.35-7.37 (m, 1H), 6.92-7.01 (m, 1H), 4.10-4.13 (m, 1H), 3.82-3.94 (m, 1H), 3.60-3.66 (m, 1H), 2.83-2.89 (m, 1H), 2.14-2.24 (m, 1H), 1.24-1.39 (m, 9H). LC-MS (ESI, m/z): 362 [M−Boc+H]⁺.

To a mixture of t-butyl (2S,4R)-4-((2-bromophenyl)difluoromethyl)-2,4-dicarbamoylpyrrolidine-1-carboxylate (160 mg, 0.346 mmol), methanesulfonato(2-dicyclohexylphosphino-2′,4′,6′-tri-i-propyl-1,1′-biphenyl)(2′-amino-1,1′-biphenyl-2-yl)palladium(II) (29.3 mg, 0.035 mmol), cesium carbonate (226 mg, 0.692 mmol, 2.0 eq.) and dicyclohexyl(2′,4′,6′-triisopropyl-[1,1′-biphenyl]-2-yl)phosphane (16.5 mg, 0.035 mmol, 0.1 eq.) was added 1,4-dioxane (2 mL) stirred under nitrogen at rt. The mixture was stirred 1 h at 90° C. The reaction was quenched with water (50 mL) and then extracted with EA (3×100 mL). The organic layers were combined, washed with brine (2×50 mL) and dried over anhydrous sodium sulfate. The solids were removed by filtration and the filtrate was concentrated under reduced pressure. The crude product was purified by prep-TLC (Mobile phase: EA); Rf=0.4; detection: UV) to provide t-butyl (3R,5S)-5-carbamoyl-4′,4′-difluoro-2′-oxo-1′,4′-dihydro-2′H-spiro[pyrrolidine-3,3′-quinoline]-1-carboxylate (90 mg, 68%). ¹H NMR (400 MHz, DMSO-d₆) δ 10.95 (s, 1H), 7.63-7.64 (m, 1H), 7.52-7.56 (m, 1H), 7.24-7.36 (m, 1H), 7.20-7.22 (m, 1H), 7.02-7.09 (m, 2H), 4.10-4.26 (m, 1H), 4.72-3.85 (m, 2H), 2.51-2.52 (m, 1H), 2.30-2.39 (m, 1H), 1.23-1.35 (m, 9H). LC-MS (ESI, m/z): 282 [M−Boc+H]⁺.

To a solution of t-butyl (3R,5S)-5-carbamoyl-4′,4′-difluoro-2′-oxo-1′,4′-dihydro-2′H-spiro[pyrrolidine-3,3′-quinoline]-1-carboxylate (90.0 mg, 0.234 mmol) in DCM (1 mL) was added TFA (0.3 mL) at rt. The mixture was stirred for 1 h at rt and concentrated under reduced pressure to afford (3R,5S)-4′,4′-difluoro-2′-oxo-1′,4′-dihydro-2′H-spiro[pyrrolidine-3,3′-quinoline]-5-carboxamide (60 mg, crude) as a brown semi-solid. LC-MS (ESI, m/z): 282 [M+H]⁺.

To a solution of (3R,5S)-4′,4′-difluoro-2′-oxo-1′,4′-dihydro-2′H-spiro[pyrrolidine-3,3′-quinoline]-5-carboxamide, (2S)-4-methyl-2-[(2S)—N-methyl-2-(2,2,2-trifluoroacetamido)propanamido]pentanoic acid (73.3 mg, 0.234 mmol) and HATU (113 mg, 0.299 mmol) in DMF (2 mL) was added N-ethyl-N-isopropylpropan-2-amine (110 mg, 0.852 mmol) at −15° C. The mixture was stirred for 2 h at −15° C. and the reaction was quenched with water (20 mL). The mixture was extracted with EA (3×50 mL). The organic layers were combined, washed with brine (2×30 mL) and dried over anhydrous sodium sulfate. The solids were removed by filtration and the filtrate was concentrated under reduced pressure. The crude product was purified by TLC (Mobile phase: MeOH:DCM=1:10; Rf=0.4; detection: UV) to afford (3R,5S)-4′,4′-difluoro-1-(N-methyl-N-((2,2,2-trifluoroacetyl)-L-alanyl)-L-leucyl)-2′-oxo-1′,4′-dihydro-2′H-spiro[pyrrolidine-3,3′-quinoline]-5-carboxamide (80.0 mg, 65%). ¹H NMR (400 MHz, DMSO-d₆) δ 10.91-11.01 (m, 1H), 9.71-9.75 (m, 1H), 7.53-7.58 (m, 2H), 7.19-7.23 (m, 1H), 7.09-7.12 (m, 1H), 6.99-7.08 (m, 2H), 5.26-5.29 (m, 1H), 4.67-4.72 (m, 1H), 4.21-4.36 (m, 1H), 4.06-4.17 (m, 2H), 2.80-2.93 (m, 3H), 2.42-2.50 (m, 1H), 2.09-2.15 (m, 1H), 1.39-1.58 (m, 3H), 1.16-1.37 (m, 3H), 0.76-0.94 (m, 6H). LC-MS (ESI, m/z): 598 [M+Na]⁺.

To a solution of (3R,5S)-4′,4′-difluoro-1-(N-methyl-N-((2,2,2-trifluoroacetyl)-L-alanyl)-L-leucyl)-2′-oxo-1′,4′-dihydro-2′H-spiro[pyrrolidine-3,3′-quinoline]-5-carboxamide (75.0 mg, 0.130 mmol) in DCM (2.5 mL) were added pyridine (41.2 mg, 0.520 mmol) and trifluoroacetic anhydride (41.1 mg, 0.195 mmol) at rt. The mixture was stirred for 0.5 h at rt and the reaction was quenched with water (20 mL). The mixture was extracted with DCM (3×80 mL). The organic layers were combined, washed with brine (2×50 mL) and dried over anhydrous sodium sulfate. The solids were removed by filtration and the filtrate was concentrated under reduced pressure. The crude product was purified by prep-HPLC (Column: Xselect CSH Prep OBD C18 Column, 30×150 mm, 5 m; Mobile Phase A: Water (0.1% FA), Mobile Phase B: CH₃CN; Flow rate: 60 mL/min; Gradient: 45% B to 60% B in 8 min; Wave Length: 254 nm/220 nm) to provide (S)—N—((S)-1-((3R,5S)-5-cyano-4′,4′-difluoro-2′-oxo-1′,4′-dihydro-2′H-spiro[pyrrolidine-3,3′-quinolin]-1-yl)-4-methyl-1-oxopentan-2-yl)-N-methyl-2-(2,2,2-trifluoroacetamido)propanamide (28.4 mg, 38%) as a white solid. ¹H NMR (400 MHz, 80° C., DMSO-d₆) δ 10.89 (br, 1H), 9.43 (br, 1H), 7.52-7.59 (m, 2H), 7.19-7.22 (m, 1H), 7.12-7.14 (m, 1H), 5.27-5.29 (m, 1H), 4.81-4.99 (m, 1H), 4.70-4.75 (m, 1H), 3.85-4.07 (m, 2H), 2.93 (s, 3H), 2.76-2.80 (m, 1H), 2.40-2.49 (m, 1H), 1.46-1.70 (m, 2H), 1.43-1.45 (m, 1H), 1.21-1.24 (m, 3H), 0.84-0.91 (m, 6H). LC-MS (ESI, m/z): 580 [M+Na]⁺.

### Example 44

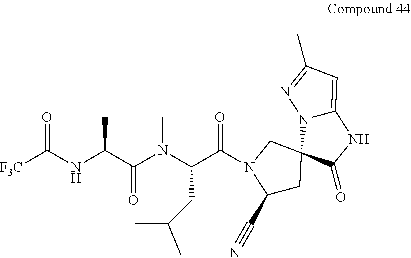 [SMILES: Cc1cc2n(n1)[C@@]1(C[C@@H](C#N)N(C(=O)[C@H](CC(C)C)N(C)C(=O)[C@H](C)NC(=O)C(F)(F)F)C1)C(=O)N2]

To a stirred mixture of t-butyl (3R,5'S)-6-bromo-5′-carbamoyl-2-oxo-1,2-dihydrospiro[imidazo[1,2-b]pyrazole-3,3′-pyrrolidine]-1′-carboxylate (400 mg, 1.01 mmol) and zinc chloride (136 mg, 1.01 mmol) in DMF (4 mL) were added (Methanesulfonato(diadamantyl-n-butylphosphino)-2′-amino-1,1′-biphenyl-2-yl)palladium(II) dichloromethane adduct (93.0 mg, 0.10 mmol, 0.1 eq.) and bis(1-adamantyl)-butylphosphane (36.0 mg, 0.10 mmol) at rt. Tetramethyltin (358 mg, 2.02 mmol) was added. The mixture was stirred overnight at 90° C. under nitrogen and the reaction was quenched with water (50 mL). The mixture was extracted with EA (3×100 mL). The organic layers were combined, dried over anhydrous magnesium sulfate. The solids were removed by filtration and the filtrate was concentrated under reduced pressure. The crude product was purified by TLC (Mobile phase: MeOH:DCM=1:9; Rf=0.4; detection: UV) to provide t-butyl (3R,5'S)-5′-carbamoyl-6-methyl-2-oxo-1,2-dihydrospiro[imidazo[1,2-b]pyrazole-3,3′-pyrrolidine]-1′-carboxylate (260 mg, 77%) as a yellow solid. LC-MS (ESI, m/z): 336 [M+H]⁺.

A mixture of t-butyl (3R,5'S)-5′-carbamoyl-6-methyl-2-oxo-1,2-dihydrospiro[imidazo[1,2-b]pyrazole-3,3′-pyrrolidine]-1′-carboxylate (120 mg, 0.357 mmol, 1.0 eq.) in hydrochloride (2 mL, 4 M in 1,4-dioxane) was stirred for 1 h at rt and then concentrated under reduced pressure to afford (3R,5'S)-6-methyl-2-oxo-1,2-dihydrospiro[imidazo[1,2-b]pyrazole-3,3′-pyrrolidine]-5′-carboxamide hydrochloride (120 mg, crude) as a yellow solid. LC-MS (ESI, m/z): 236 [M+H]⁺.

To a stirred mixture of (3R,5'S)-6-methyl-2-oxo-1,2-dihydrospiro[imidazo[1,2-b]pyrazole-3,3′-pyrrolidine]-5′-carboxamide hydrochloride (120 mg, 0.443 mmol), N-methyl-N-((2,2,2-trifluoroacetyl)-L-alanyl)-L-leucine (143 mg, 0.456 mmol) and HATU (202 mg, 0.532 mmol) in DMF (2 mL) was added N-ethyl-N-isopropylpropan-2-amine (114 mg, 0.886 mmol). The mixture was stirred for 40 mins at −15° C. and the reaction was quenched with water (8 mL). The mixture was extracted with EA (3×50 mL). The organic layers were combined, washed with brine (2×20 mL) and dried over anhydrous sodium sulfate. The solids were removed by filtration and the filtrate was concentrated under reduced pressure. The crude product was purified by TLC (Mobile phase: MeOH/DCM=1:10; Rf=0.4; detection: UV) to afford (3R,5'S)-6-methyl-1′-(N-methyl-N-((2,2,2-trifluoroacetyl)-L-alanyl)-L-leucyl)-2-oxo-1,2-dihydrospiro[imidazo[1,2-b]pyrazole-3,3′-pyrrolidine]-5′-carboxamide (70.0 mg, 30%) as a white solid. LC-MS (ESI, m/z): 530 [M+H]⁺.

To a mixture of (3R,5'S)-6-methyl-1′-(N-methyl-N-((2,2,2-trifluoroacetyl)-L-alanyl)-L-leucyl)-2-oxo-1,2-dihydrospiro[imidazo[1,2-b]pyrazole-3,3′-pyrrolidine]-5′-carboxamide (70.0 mg, 0.132 mmol) in DCM (1 mL) were added pyridine (53.0 mg, 0.742 mmol) and trifluoroacetic anhydride (41.6 mg, 0.198 mmol). The mixture was stirred for 1 h at rt and the reaction was quenched with water (10 mL). The mixture was extracted with DCM (3×20 mL). The organic layers were combined, washed with brine (2×10 mL) and dried over anhydrous sodium sulfate. The solids were removed by filtration and the filtrate was concentrated under reduced pressure. The crude product was purified by prep-HPLC (Column: Xselect CSH Prep OBD C18 Column 30×150 mm, 5 m; Mobile Phase A: Water (0.1% FA), Mobile Phase B: ACN; Flow rate: 60 mL/min; Gradient: 31% B to 46% B in 8 min; Wave Length: 220 nm) to provide (S)—N—((S)-1-((3R,5'S)-5′-cyano-6-methyl-2-oxo-1,2-dihydrospiro[imidazo[1,2-b]pyrazole-3,3′-pyrrolidin]-1′-yl)-4-methyl-1-oxopentan-2-yl)-N-methyl-2-(2,2,2-trifluoroacetamido)propanamide (16.5 mg, 25%) as a white solid. ¹H NMR (400 MHz, 80° C., DMSO-d₆) δ 11.32 (br, 1H), 9.32 (s, 1H), 7.51 (s, 1H), 5.05-5.22 (m, 2H), 4.60 (s, 1H), 3.55-4.18 (m, 2H), 2.90 (s, 3H), 2.65-2.85 (m, 1H), 2.55-2.63 (m, 1H), 2.12 (s, 3H), 1.27-1.80 (m, 3H), 1.00-1.13 (m, 6H), 0.79-0.99 (m, 3H). LC-MS (ESI, m/z): 512 [M+H]⁺.

### Example 45

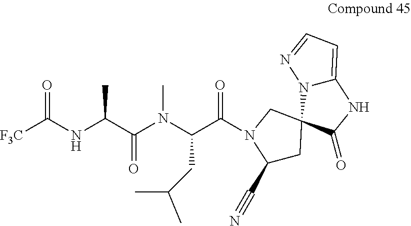 [SMILES: CC(C)C[C@@H](C(=O)N1C[C@]2(C[C@H]1C#N)C(=O)Nc1ccnn12)N(C)C(=O)[C@H](C)NC(=O)C(F)(F)F]

To a mixture of (3R,5'S)-6-bromo-1′-[(2S)-4-methyl-2-[(2S)—N-methyl-2-(2,2,2-trifluoroacetamido)propanamido]pentanoyl]-2-oxo-1H-spiro[pyrazolo[1,5-a]imidazole-3,3′-pyrrolidine]-5′-carboxamide (150 mg, 0.252 mmol) in THF (5 mL) were added potassium carbonate (53.0 mg, 0.378 mmol) and 10% Pd on activated carbon (75.0 mg). The mixture was stirred for 3 h at 50° C. under hydrogen. The mixture was filtered through a celite pad and washed with THF (3×5 mL). The filtrate was concentrated under reduced pressure to afford (3R,5'S)-1′-[(2S)-4-methyl-2-[(2S)—N-methyl-2-(2,2,2-trifluoroacetamido)propanamido]pentanoyl]-2-oxo-1H-spiro[pyrazolo[1,5-a]imidazole-3,3′-pyrrolidine]-5′-carboxamide (120 mg, crude) as a white solid. LC-MS (ESI, m/z): 516 [M+H]⁺.

To a mixture of (3R,5'S)-1′-[(2S)-4-methyl-2-[(2S)—N-methyl-2-(2,2,2-trifluoroacetamido)propanamido]pentanoyl]-2-oxo-1H-spiro[pyrazolo[1,5-a]imidazole-3,3′-pyrrolidine]-5′-carboxamide (100 mg, 0.194 mmol) in DCM (5 mL) were added pyridine (76.7 mg, 0.970 mmol) and trifluoroacetic anhydride (73.0 mg, 0.349 mmol). The mixture was stirred for 1 h at rt and the reaction was quenched with water (10 mL). The mixture was extracted with EA (3×10 mL). The organic layers were combined, washed with brine (2×10 mL) and dried over anhydrous sodium sulfate. The solids were removed by filtration and the filtrate was concentrated under reduced pressure to remove the solvent. The residue was chromatographed on a silica gel column with MeOH:DCM (5/95) to afford the crude product. The product was purified by prep-Achiral-HPLC (Column: Viridis BEH Prep 2-EP OBD Column 3×15 cm, Sum; Mobile Phase A: CO₂, Mobile Phase B: IPA; Flow rate: 65 mL/min; Gradient: isocratic 24% B; Column Temperature(° C.): 35; Back Pressure(bar): 100; Wave Length: 220 nm): 3.16; Sample Solvent: CH₃CN) to provide (2S)—N-[(2S)-1-[(3R,5'S)-5′-cyano-2-oxo-1H-spiro[pyrazolo[1,5-a]imidazole-3,3′-pyrrolidin]-1′-yl]-4-methyl-1-oxopentan-2-yl]-N-methyl-2-(2,2,2-trifluoroacetamido)propanamide (12.9 mg, 13%) as a white solid. ¹H NMR (400 MHz, 80° C., DMSO-d₆) δ 10.80-11.90 (m, 1H), 9.31 (br, 1H), 7.30-7.45 (m, 1H), 5.60-5.75 (m, 1H), 4.96-5.30 (m, 2H), 4.50-4.70 (m, 1H), 3.65-4.15 (m, 2H), 2.70-2.95 (m, 3H), 2.55-2.69 (m, 2H), 1.40-1.80 (m, 3H), 0.60-1.10 (m, 9H). LC-MS (ESI, m/z): 520 [M+Na]⁺.

### Example 46

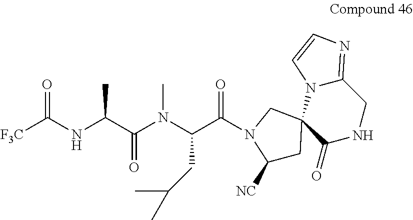 [SMILES: CC(C)C[C@@H](C(=O)N1C[C@]2(C[C@H]1C#N)C(=O)NCc1nccn12)N(C)C(=O)[C@H](C)NC(=O)C(F)(F)F]

To a solution of zinc cyanide (228 mg, 1.94 mmol, 1.4 eq.), zinc powder (10.9 mg, 0.167 mmol), 1,1′-Bis(diphenylphosphino)ferrocene (169 mg, 0.305 mmol) and tris(dibenzylideneacetone)dipalladium (165 mg, 0.180 mmol) in DMA (5 mL) was added 1-t-butyl 2,4-dimethyl (2S,4R)-4-(2-bromoimidazol-1-yl)pyrrolidine-1,2,4-tricarboxylate (600 mg, 1.39 mmol, in 2 mL DMA) at rt under nitrogen. The mixture was stirred 1 h at 120° C. and the reaction was quenched with water (30 mL). The mixture was extracted with EA (3×100 mL). The organic layers were combined, washed with brine (3×80 mL) and dried over anhydrous sodium sulfate. The solids were removed by filtration and the filtrate was concentrated under reduced pressure. The crude product was purified by TLC (Mobile phase: EA:PE=1.2:1; Rf=0.4; detection: UV) to provide 1-t-butyl 2,4-dimethyl (2S,4R)-4-(2-cyanoimidazol-1-yl)pyrrolidine-1,2,4-tricarboxylate (170 mg, 32%) as a yellow solid. ¹H NMR (400 MHz, DMSO-d₆) δ 7.90-7.93 (m, 1H), 7.31 (s, 1H), 4.43-4.51 (m, 1H), 4.28-4.39 (m, 1H), 4.13-4.21 (m, 1H), 3.64-3.76 (m, 6H), 3.34-3.42 (m, 1H), 2.90-3.05 (m, 1H), 1.32-1.34 (m, 9H). LC-MS (ESI, m/z): 379 [M+H]⁺.

To a solution of 1-t-butyl 2,4-dimethyl (2S,4R)-4-(2-cyanoimidazol-1-yl)pyrrolidine-1,2,4-tricarboxylate (170 mg, 0.449 mmol) in MeOH (5 mL) was added 10% Pd on activated carbon (90 mg) at rt. The mixture was stirred overnight at rt under hydrogen. The mixture was filtered through a celite pad and washed with MeOH (3×50 mL). The filtrate was concentrated under reduced pressure. The crude product was purified by C18 column with CH₃CN/Water (0.05% TFA). The fraction was concentrated under reduced pressure to provide 1′-t-butyl 5′-methyl (5R,5'S)-6-oxo-7,8-dihydrospiro[imidazo[1,2-a]pyrazine-5,3′-pyrrolidine]-1′,5′-dicarboxylate (50 mg, 31%) as a light yellow solid. LC-MS (ESI, m/z): 351 [M+H]*.

A mixture of 1′-t-butyl 5′-methyl (5R,5'S)-6-oxo-7,8-dihydrospiro[imidazo[1,2-a]pyrazine-5,3′-pyrrolidine]-1′,5′-dicarboxylate (50.0 mg, 0.143 mmol) and ammonia (3 mL, 7 M in MeOH) was stirred for 24 h at rt and then concentrated under reduced pressure to afford tert-butyl (5R,5'S)-5′-carbamoyl-6-oxo-7,8-dihydro-6H-spiro[imidazo[1,2-a]pyrazine-5,3′-pyrrolidine]-1′-carboxylate (50 mg, crude) as a white solid. LC-MS (ESI, m/z): 336 [M+H]⁺.

A mixture of t-butyl (5R,5'S)-5′-carbamoyl-6-oxo-7,8-dihydro-6H-spiro[imidazo[1,2-a]pyrazine-5,3′-pyrrolidine]-1′-carboxylate (50.0 mg, 0.149 mmol) in DCM (0.9 mL) was added TFA(0.3 mL). The mixture was stirred for 1 h at rt and then concentrated under reduced pressure to afford (5R,5'S)-6-oxo-7,8-dihydro-6H-spiro[imidazo[1,2-a]pyrazine-5,3′-pyrrolidine]-5′-carboxamide (50 mg, crude) as a yellow oil. LC-MS (ESI, m/z): 236 [M+H]⁺.

To a stirred mixture of (5R,5'S)-6-oxo-7,8-dihydro-6H-spiro[imidazo[1,2-a]pyrazine-5,3′-pyrrolidine]-5′-carboxamide (50.0 mg, 0.212 mmol), N-methyl-N-((2,2,2-trifluoroacetyl)-L-alanyl)-L-leucine (68.4 mg, 0.218 mmol) and HATU (96.7 mg, 0.254 mmol) in dimethylformamide (2 mL) was added N-ethyl-N-isopropylpropan-2-amine (54.8 mg, 0.424 mmol) at −15° C. The mixture was stirred for 1 h at −15° C. and the reaction was quenched with water (5 mL). The mixture was extracted with EA (3×30 mL). The organic layers were combined, washed with brine (2×10 mL), dried over anhydrous sodium sulfate. The solids were removed by filtration and the filtrate was concentrated under reduced pressure. The crude product was purified by TLC (Mobile phase: MeOH:DCM=1:9; Rf=0.4; detection: UV) to provide (5R,5'S)-1′-(N-methyl-N-((2,2,2-trifluoroacetyl)-L-alanyl)-L-leucyl)-6-oxo-7,8-dihydro-6H-spiro[imidazo[1,2-a]pyrazine-5,3′-pyrrolidine]-5′-carboxamide (40 mg, crude) as a yellow oil. LC-MS (ESI, m/z): 530 [M+H]⁺.

To a mixture of (5R,5'S)-1′-(N-methyl-N-((2,2,2-trifluoroacetyl)-L-alanyl)-L-leucyl)-6-oxo-7,8-dihydro-6H-spiro[imidazo[1,2-a]pyrazine-5,3′-pyrrolidine]-5′-carboxamide (40.0 mg, 0.075 mmol, 1.0 eq.) in DCM (1 mL) was added burgess reagent (215.0 mg, 0.605 mmol). The mixture was stirred for 1 h at rt. The reaction was quenched with water (5 mL) and then extracted with EA (3×20 mL). The organic layers were combined, washed with brine (2×10 mL), dried over anhydrous sodium sulfate. The solids were removed by filtration and the filtrate was concentrated under reduced pressure. The crude product was purified by prep-HPLC (Column: Xselect CSH Prep OBD C18 Column 30×150 mm, 5 m; Mobile Phase A: Water (0.1% FA), Mobile Phase B: ACN; Flow rate: 60 mL/min; Gradient: 10% B to 23% B in 11 min; Wave Length: 220 nm) to provide (S)—N—((S)-1-((5R,5'S)-5′-cyano-6-oxo-7,8-dihydro-6H-spiro[imidazo[1,2-a]pyrazine-5,3′-pyrrolidin]-1′-yl)-4-methyl-1-oxopentan-2-yl)-N-methyl-2-(2,2,2-trifluoroacetamido)propanamide (3.5 mg, 9%) as a white solid. ¹H NMR (400 MHz, 60° C., DMSO-d₆) δ 9.48 (br, 1H), 8.70 (s, 1H), 6.78-7.25 (m, 2H), 5.02-5.48 (m, 2H), 4.63-4.91 (m, 1H), 4.32-4.60 (m, 2H), 3.85-4.35 (m, 2H), 2.93-3.07 (m, 1H), 2.74-2.96 (m, 4H), 1.33-1.76 (m, 3H), 1.01-1.32 (m, 3H), 0.67-1.00 (m, 6H). LC-MS (ESI, m/z): 512 [M+H]⁺.

The compounds provided in Table 1 were synthesized using similar methods as those described herein.

TABLE 1

| | | LC-MS | |
|---|---|---|---|
| Entry | Structure | (ESI, m/z) | NMR |
| 47 | 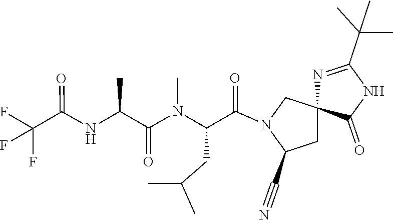 [SMILES: CC(C)C[C@@H](C(=O)N1C[C@@]2(C[C@H]1C#N)N=C(C(C)(C)C)NC2=O)N(C)C(=O)[C@H](C)NC(=O)C(F)(F)F] | 515 [M + H]⁺ | ¹H NMR (400 MHz, 80° C., DMSO-d₆) δ 10.79-11.12 (m, 1H), 9.17-9.61 (m, 1H), 5.11- 5.51 (m, 1H), 4.81-5.09 (m, 1H), 4.61-4.80 (m, 1H), 3.31-3.86 (m, 2H), 2.78-3.02 (m, 3H), 2.21- 2.46 (m, 2H), 1.37-1.81 (m, 3H), 1.20-1.36 (m, 3H), 1.05-1.19 (m, 9H), 0.76-0.98 (m, 6H). |
| 48 | 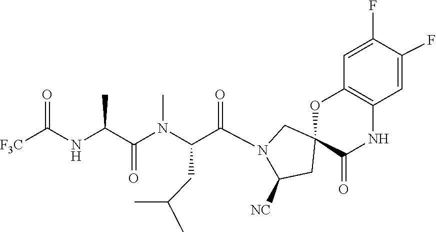 [SMILES: CC(C)C[C@@H](C(=O)N1C[C@@]2(C[C@H]1C#N)Oc1cc(F)c(F)cc1NC2=O)N(C)C(=O)[C@H](C)NC(=O)C(F)(F)F] | 582 [M + Na]⁺ | ¹H NMR (400 MHz, 80° C., DMSO-d₆) δ 10.93 (br, 1H), 9.36 (br, 1H), 7.01-7.20 (m, 1H), 6.80- 7.00 (m, 1H), 4.81-5.30 (m, 2H), 4.58-4.80 (m, 1H), 3.83-4.20 (m, 1H), 3.46-3.82 (m, 1H), 2.84- 3.02 (m, 3H), 2.57-2.83 (m, 2H), 1.33-1.75 (m, 3H), 1.06-1.32 (m, 3H), 0.60-1.05 (m, 6H) |
| 49 | 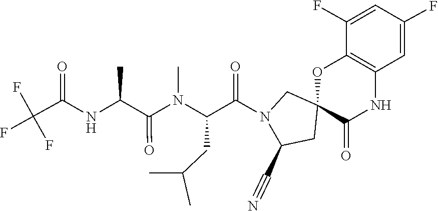 [SMILES: CC(C)C[C@@H](C(=O)N1C[C@@]2(C[C@H]1C#N)Oc1c(F)cc(F)cc1NC2=O)N(C)C(=O)[C@H](C)NC(=O)C(F)(F)F] | 582 [M + Na]⁺ | ¹H NMR (400 MHz, 80° C., DMSO-d₆) δ 8.80-9.80 (m, 1H), 6.70-7.00 (m, 1H), 6.50-6.70 (m, 1H), 4.70-5.50 (m, 2H), 4.50- 4.70 (m, 1H), 3.85-4.15 (m, 2H), 2.88-2.93 (m, 3H), 2.75-2.83 (m, 1H), 2.55-2.69 (m, 1H), 1.48- 1.80 (m, 2H), 1.30-1.48 (m, 1H), 0.95-1.30 (m, 3H), 0.70-0.95 (m, 6H) |
| 50 | 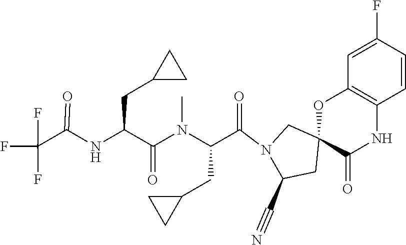 [SMILES: CN(C(=O)[C@H](CC1CC1)NC(=O)C(F)(F)F)[C@@H](CC1CC1)C(=O)N1C[C@@]2(C[C@H]1C#N)Oc1cc(F)ccc1NC2=O] | 580 [M + H]⁺ | ¹H NMR (400 MHz, 80° C., DMSO-d₆) δ 10.80 (br, 1H), 9.40 (br, 1H), 6.98-7.06 (m, 1H), 6.73- 6.89 (m, 2H), 5.21 (br, 1H), 4.94 (br, 1H), 4.68-4.85 (m, 1H), 4.00- 4.18 (m, 2H), 2.97-3.04 (m, 3H), 2.73-3.85 (m, 1H), 2.53-2.70 (m, 1H), 1.60-1.74 (m, 2H), 1.39- 1.59 (m, 2H), 0.55-0.70 (m, 2H), 0.33-0.57 (m, 4H), 0.01-0.14 (m, 3H), −0.90-0.00 (m, 1H) |
| 51 | 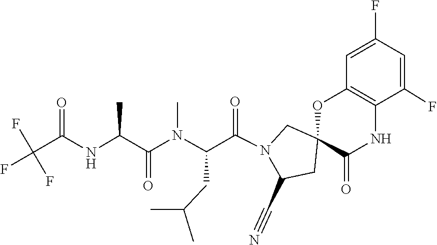 | 560 [M + H]⁺ | ¹H NMR (400 MHz, 80º C., DMSO-d₆) δ 10.30-11.60 (m, 0.3H), 9.10-9.60 (m, 1H), 6.80- 7.00 (m, 1H), 6.50-6.80 (m, 1H), 4.80-5.30 (m, 2H), 4.50-4.80 (m, 1H), 3.30-4.10 (m, 2H), 2.50- 2.90 (m, 5H), 1.00-1.70 (m, 6H), 0.50-1.00 (m, 6H) |
| 52 | 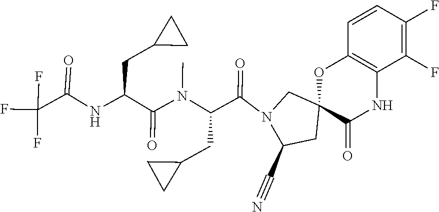 [SMILES: CN(C(=O)[C@H](CC1CC1)NC(=O)C(F)(F)F)[C@@H](CC1CC1)C(=O)N1C[C@@]2(C[C@H]1C#N)Oc1ccc(F)c(F)c1NC2=O] | 620 [M + Na]⁺ | ¹H NMR (400 MHz, 80° C., DMSO-d₆) δ 9.20-9.60 (m, 1H), 6.80-7.00 (m, 2H), 5.24 (br, 1H), 4.90 (br, 1H), 4.77 (br, 1H), 4.14 (br, 2H), 2.90-3.00 (m, 3H), 2.80- 2.90 (m, 1H), 2.60-2.80 (m, 1H), 1.20-1.80 (m, 4H), 0.50-0.70 (m, 2H), −0.80-0.45 (m, 8H) |
| 53 | 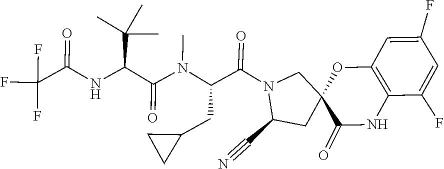 [SMILES: CN(C(=O)[C@@H](NC(=O)C(F)(F)F)C(C)(C)C)[C@@H](CC1CC1)C(=O)N1C[C@@]2(C[C@H]1C#N)Oc1cc(F)cc(F)c1NC2=O] | 600 [M + H]⁺ | ¹H NMR (400 MHz, 80° C., DMSO-d₆) δ 11.15 (br, 1H), 8.80- 9.15 (m, 1H), 6.82-7.00 (m, 1H), 6.50-6.69 (m, 1H), 5.05-5.25 (m, 1H), 4.79-5.00 (m, 1H), 4.60- 4.78 (m, 1H), 3.90-4.22 (m, 2H), 2.99 (s, 3H), 2.65-2.85 (m, 1H), 2.50-2.64 (m, 1H), 1.45-1.75 (m, 2H), 0.61-1.05 (m, 9H), 0.35- 0.59 (m, 1H), 0.10-0.34 (m, 2H), −0.10-0.09 (m, 2H) |
| 54 | 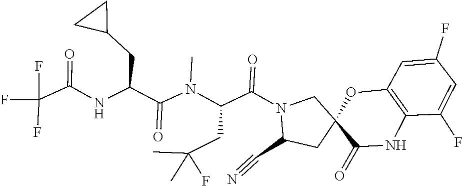 | 640 [M + Na]⁺ | ¹H NMR (400 MHz, 80° C., DMSO-d₆) δ 10.45-11.55 (m, 1H), 9.01-9.58 (m, 1H), 6.80- 7.02 (m, 1H), 6.50-6.79 (m, 1H), 5.20-5.60 (m, 1H), 4.60-5.19 (m, 2H), 3.80-4.32 (m, 2H), 2.91- 2.95 (m, 3H), 2.45-2.82 (m, 2H), 2.10-2.39 (m, 1H), 1.80-2.03 (m, 1H), 1.40-1.75 (m, 2H), 1.12- 1.39 (m, 6H), 0.50-0.70 (m, 1H), 0.20-0.42 (m, 2H), 0.00-0.10 (m, 1H), −0.11 − −0.04 (m, 1H) |
| 55 | 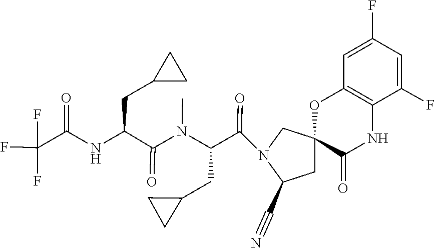 [SMILES: CN(C(=O)[C@H](CC1CC1)NC(=O)C(F)(F)F)[C@@H](CC1CC1)C(=O)N1C[C@@]2(C[C@H]1C#N)Oc1cc(F)cc(F)c1NC2=O] | 333 [fragment peak] | ¹H NMR (400 MHz, 80° C., DMSO-d₆) δ 10.30-11.80 (m, 1H), 9.28 (br, 1H), 6.81-7.31 (m, 1H), 6.33-6.79 (m, 1H), 5.01- 5.61 (m, 1H), 4.56-4.98 (m, 2H), 3.89-4.55 (m, 2H), 2.93 (s, 3H), 2.65-2.89 (m, 1H), 2.51-2.64 (m, 1H), 1.26-1.70 (m, 4H), 0.45- 0.65 (m, 2H), 0.11-0.44 (m, 4H), −0.25-0.10 (m, 4H) |
| 56 | 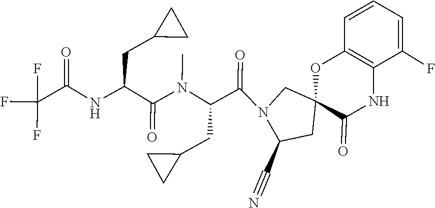 [SMILES: CN(C(=O)[C@H](CC1CC1)NC(=O)C(F)(F)F)[C@@H](CC1CC1)C(=O)N1C[C@@]2(C[C@H]1C#N)Oc1cccc(F)c1NC2=O] | 602 [M + Na]⁺ | ¹H NMR (400 MHz, 80° C., DMSO-d₆) δ 11.04 (br, 1H), 9.34 (s, 1H), 6.82-7.09 (m, 2H), 6.61- 6.81 (m, 1H), 5.07-5.38 (m, 1H), 4.81-5.05 (m, 1H), 4.53-4.80 (m, 1H), 3.85-4.29 (m, 2H), 2.98 (s, 3H), 2.68-2.81 (m, 1H), 2.50- 2.67 (m, 1H), 1.54-1.80 (m, 2H), 1.20-1.53 (m, 2H), 0.48-0.70 (m, 2H), 0.20-0.46 (m, 4H), 0.02- 0.19 (m, 2H), −0.04-0.01 (m, 1H), −0.09 − −0.05 (m, 1H) |
| 57 | 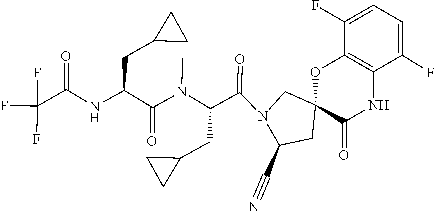 [SMILES: CN(C(=O)[C@H](CC1CC1)NC(=O)C(F)(F)F)[C@@H](CC1CC1)C(=O)N1C[C@@]2(C[C@H]1C#N)Oc1c(F)ccc(F)c1NC2=O] | 598 [M + H]⁺ | ¹H NMR (400 MHz, 100° C., DMSO-d₆) δ 11.30 (br, 1H), 9.20 (s, 1H), 6.82-7.03 (m, 2H), 5.12- 5.48 (m, 1H), 4.85-5.47 (m, 1H), 4.67-4.84 (m, 1H), 4.00-4.40 (m, 2H), 2.99-3.06 (m, 3H), 2.77- 2.91 (m, 1H), 2.58-2.76 (m, 1H), 1.43-1.78 (m, 4H), 0.50-0.81 (m, 2H), 0.20-0.80 (m, 4H), 0.01- 0.19 (m, 3H), −0.80 − −0.98 (m, 1H) |
| 58 | 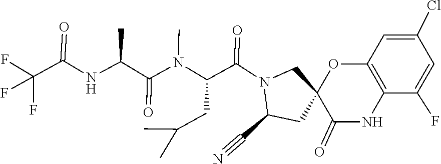 [SMILES: CC(C)C[C@@H](C(=O)N1C[C@@]2(C[C@H]1C#N)Oc1cc(Cl)cc(F)c1NC2=O)N(C)C(=O)[C@H](C)NC(=O)C(F)(F)F] | 598 [M + Na]⁺ | ¹H NMR (400 MHz, 80° C., DMSO-d₆) δ 11.22 (s, 1H), 9.34 (s, 1H), 7.05-7.25 (m, 1H), 6.93 (s, 1H), 4.80-5.40 (m, 2H), 4.58- 4.79 (m, 1H), 3.80-4.35 (m, 2H), 2.95 (s, 3H), 2.55-2.87 (m, 2H), 1.55-1.65 (m, 2H), 1.39-1.54 (m, 1H), 1.10-1.30 (m, 3H), 0.60- 1.01 (m, 6H) |
| 59 | 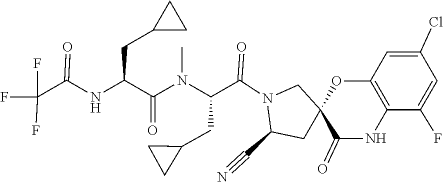 [SMILES: CN(C(=O)[C@H](CC1CC1)NC(=O)C(F)(F)F)[C@@H](CC1CC1)C(=O)N1C[C@@]2(C[C@H]1C#N)Oc1cc(Cl)cc(F)c1NC2=O] | 636 [M + Na]⁺ | ¹H NMR (400 MHz, 80° C., DMSO-d₆) δ 10.65-11.68 (m, 1H), 9.26 (br, 1H), 6.94-7.15 (m, 1H), 6.80 (s, 1H), 5.00-5.30 (m, 1H), 4.55-4.99 (m, 2H), 3.90- 4.25 (m, 2H), 2.99 (s, 3H), 2.60- 2.80 (m, 1H), 2.45-2.59 (m, 1H), 1.50-1.80 (m, 2H), 1.30-1.49 (m, 2H), 0.45-0.70 (m, 2H), 0.20- 0.40 (m, 4H), −0.05-0.10 (m, 3H), −0.25 − −0.06 (m, 1H) |
| 60 | 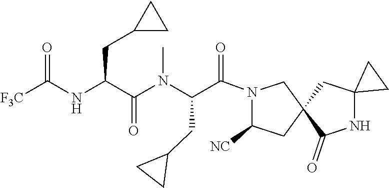 [SMILES: CN(C(=O)[C@H](CC1CC1)NC(=O)C(F)(F)F)[C@@H](CC1CC1)C(=O)N1C[C@@]2(C[C@H]1C#N)CC1(CC1)NC2=O] | 524 [M + H]⁺ | ¹H NMR (500 MHz, 370K, DMSO-d₆) δ 9.20 (br. s., 1H), 7.73 (s, 1H), 5.16 (m, 1H), 4.82 (m, 1H), 4.74 (m, 1H), 3.92 (m, 1H), 3.65 (m, 1H), 3.04 (s, 3H), 2.42-2.50 (m, 1H), 2.38 (m, 1H), 1.93-2.02 (m, 2H), 1.61-1.70 (m, 4H), 0.75 (m, 2H), 0.59-0.71 (m, 4H), 0.34-0.46 (m, 4H), 0.08- 0.18 (m, 3H), 0.01-0.05 (m, 1H) |
| 61 | 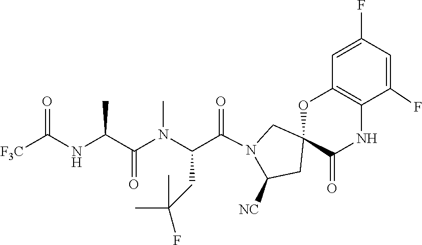 [SMILES: C[C@H](NC(=O)C(F)(F)F)C(=O)N(C)[C@@H](CC(C)(C)F)C(=O)N1C[C@@]2(C[C@H]1C#N)Oc1cc(F)cc(F)c1NC2=O] | 600 [M + Na]⁺ | ¹H NMR (400 MHz, 80° C., DMSO-d₆) δ 11.22 (br, 1H), 9.46 (s, 1H), 6.90-7.10 (m, 1H), 6.46- 6.89 (m, 1H), 5.20-5.50 (m, 1H), 4.80-5.13 (m, 1H), 4.60-4.79 (m, 1H), 4.10-4.22 (m, 1H), 3.80- 4.09 (m, 1H), 2.97 (s, 3H), 2.75- 2.89 (m, 1H), 2.60-2.74 (m, 1H), 2.20-2.40 (m, 1H), 1.80-2.18 (m, 1H), 1.27-1.33 (m, 3H), 1.22- 1.26 (m, 3H), 1.08-1.16 (m, 3H) |
| 62 | 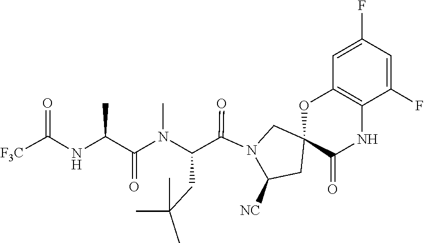 [SMILES: C[C@H](NC(=O)C(F)(F)F)C(=O)N(C)[C@@H](CC(C)(C)C)C(=O)N1C[C@@]2(C[C@H]1C#N)Oc1cc(F)cc(F)c1NC2=O] | 596 [M + Na]⁺ | ¹H NMR (400 MHz, 80° C., DMSO-d₆) δ 11.25 (br, 1H), 9.46 (s, 1H), 6.90-7.10 (m, 1H), 6.60- 6.89 (m, 1H), 5.10-5.40 (m, 1H), 4.80-5.08 (m, 1H), 4.55-4.78 (m, 1H), 3.90-4.25 (m, 2H), 2.96 (s, 3H), 2.55-2.89 (m, 2H), 1.80- 2.10 (m, 1H), 1.30-1.55 (m, 1H), 1.05-1.28 (m, 3H), 0.70-1.03 (m, 9H) |
| 63 | 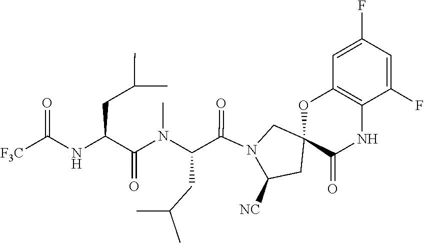 [SMILES: CC(C)C[C@H](NC(=O)C(F)(F)F)C(=O)N(C)[C@@H](CC(C)C)C(=O)N1C[C@@]2(C[C@H]1C#N)Oc1cc(F)cc(F)c1NC2=O] | 624 [M + Na]⁺ | ¹H NMR (400 MHz, 80° C., DMSO-d₆) δ 11.13 (br, 1H), 9.43 (s, 1H), 6.86-7.17 (m, 1H), 6.51- 6.85 (m, 1H), 5.11-5.49 (m, 1H), 4.81-5.09 (m, 1H), 4.50-4.79 (m, 1H), 3.89-4.44 (m, 2H), 2.97 (s, 3H), 2.71-2.89 (m, 1H), 2.51- 2.69 (m, 1H), 1.49-1.92 (m, 4H), 1.11-1.47 (m, 2H), 0.41-1.09 (m, 12H) |
| 64 | 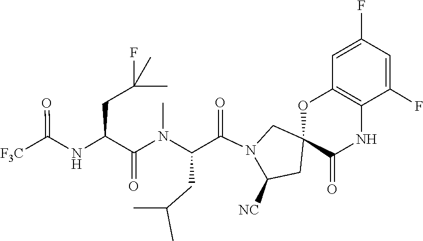 [SMILES: CC(C)C[C@@H](C(=O)N1C[C@@]2(C[C@H]1C#N)Oc1cc(F)cc(F)c1NC2=O)N(C)C(=O)[C@H](CC(C)(C)F)NC(=O)C(F)(F)F] | 642 [M + Na]⁺ | ¹H NMR (400 MHz, 80° C., DMSO-d₆) δ 11.10 (s, 1H), 9.57 (s, 1H), 6.86-7.12 (m, 1H), 6.56- 6.84 (m, 1H), 5.07-5.46 (m, 1H), 4.73-5.06 (m, 2H), 4.11-4.37 (m, 1H), 3.89-4.09 (m, 1H), 2.99 (s, 3H), 2.52-2.91 (m, 2H), 1.89- 2.27 (m, 2H), 1.51-1.69 (m, 2H), 1.34-1.59 (m, 1H), 1.02-1.32 (m, 6H), 0.59-0.99 (m, 6H) |
| 65 | 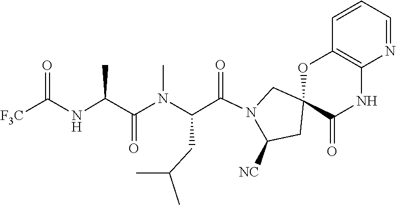 [SMILES: CC(C)C[C@@H](C(=O)N1C[C@@]2(C[C@H]1C#N)Oc1cccnc1NC2=O)N(C)C(=O)[C@H](C)NC(=O)C(F)(F)F] | 525 [M + H]⁺ | ¹H NMR (400 MHz, 80° C., DMSO-d₆) δ 11.35 (br, 1H), 9.33 (br, 1H), 7.90-8.15 (m, 1H), 7.20- 7.50 (m, 1H), 6.80-7.18 (m, 1H), 4.90-5.55 (m, 2H), 4.60-4.86 (m, 1H), 3.55-4.30 (m, 2H), 2.70- 3.00 (m, 5H), 1.48-1.90 (m, 2H), 1.10-1.46 (m, 4H), 0.50-1.08 (m, 6H) |
| 66 | 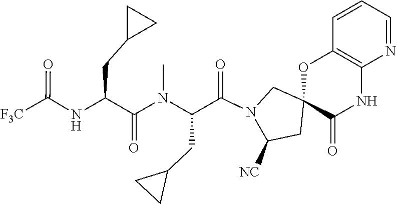 [SMILES: CN(C(=O)[C@H](CC1CC1)NC(=O)C(F)(F)F)[C@@H](CC1CC1)C(=O)N1C[C@@]2(C[C@H]1C#N)Oc1cccnc1NC2=O] | 585 [M + Na]⁺ | ¹H NMR (400 MHz, 80° C., DMSO-d₆) δ 11.30 (br, 1H), 9.28 (br, 1H), 7.80-7.95 (m, 1H), 7.12- 7.32 (m, 1H), 6.85-7.05 (m, 1H), 5.00-5.30 (m, 1H), 4.80-4.99 (m, 1H), 4.50-4.79 (m, 1H), 3.80- 4.20 (m, 2H), 2.93 (s, 3H), 2.50- 2.90 (m, 2H), 1.50-1.80 (m, 2H), 1.20-1.49 (m, 2H), 0.40-0.70 (m, 2H), 0.13-0.39 (m, 4H), −0.05- 0.12 (m, 3H), −0.25 − −0.04 (m, 1H). |
| 67 | 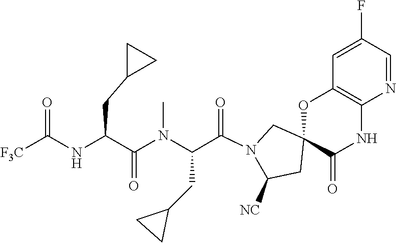 [SMILES: CN(C(=O)[C@H](CC1CC1)NC(=O)C(F)(F)F)[C@@H](CC1CC1)C(=O)N1C[C@@]2(C[C@H]1C#N)Oc1cc(F)cnc1NC2=O] | 603 [M + Na]⁺ | ¹H NMR (400 MHz, 80º C, DMSO-d₆) δ 10.40-11.60 (m, 0.35H), 8.80-10.40 (m, 1H), 7.80-8.00 (m, 1H), 7.10-7.50 (m, 1H), 5.00-5.40 (m, 1H), 4.80- 4.99 (m, 1H), 4.40-4.79 (m, 1H), 3.80-4.30 (m, 2H), 3.00 (s, 3H), 2.50-2.80 (m, 2H), 1.10-1.85 (m, 4H), 0.40-0.70 (m, 2H), 0.20- 0.39 (m, 4H), −0.10-0.19 (m, 3H), −0.35 − −0.11 (m, 1H) |
| 68 | 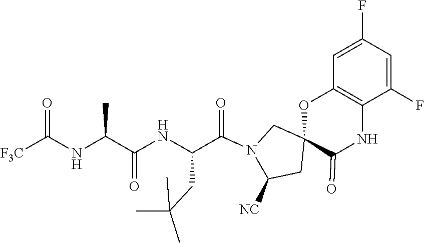 [SMILES: C[C@H](NC(=O)C(F)(F)F)C(=O)N[C@@H](CC(C)(C)C)C(=O)N1C[C@@]2(C[C@H]1C#N)Oc1cc(F)cc(F)c1NC2=O] | 568 [M + Na]⁺ | ¹H NMR (400 MHz, DMSO-d₆) δ 8.10- 8.30 (m, 1H), 6.91-7.11(m, 1H) 6.71-6.81 (m, 1H), 4.81-5.21 (m, 1H), 4.31-4.51 (m, 2H), 4.10- 4.28 (m, 1H), 3.91-4.08 (m, 1H), 2.71-2.91 (m, 2H), 1.61-1.81(m, 1H), 1.51-1.58 (m, 2H), 1.21- 1.40 (m, 3H), 0.80-1.00 (m, 6H) |
| 69 | 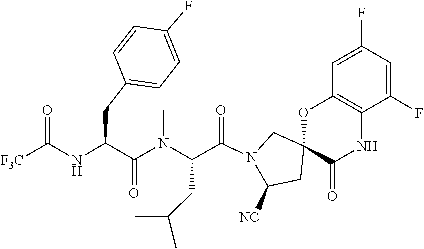 [SMILES: CC(C)C[C@@H](C(=O)N1C[C@@]2(C[C@H]1C#N)Oc1cc(F)cc(F)c1NC2=O)N(C)C(=O)[C@H](Cc1ccc(F)cc1)NC(=O)C(F)(F)F] | 676 [M + Na]⁺ | ¹H NMR (400 MHz, 80° C., DMSO-d₆) δ 9.56 (br, 1H), 7.15- 7.50 (m, 2H), 6.95-7.14 (m, 2H), 6.80-6.94 (m, 1H), 6.58-6.79 (m, 1H), 5.10-5.60 (m, 2H), 4.75- 5.08 (m, 1H), 4.00-4.30 (m, 2H), 2.85-2.95 (m, 3H), 2.72-2.84 (m, 3H), 2.60-2.71 (m, 1H), 1.50- 1.80 (m, 2H), 1.20-1.49 (m, 1H), 0.90-1.10 (m, 3H), 0.70-0.89 (m, 3H) |
| 70 | 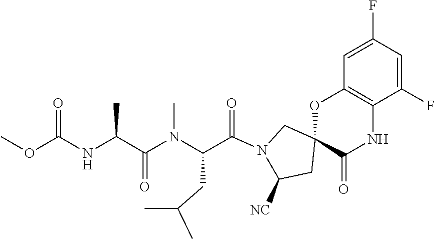 [SMILES: COC(=O)N[C@@H](C)C(=O)N(C)[C@@H](CC(C)C)C(=O)N1C[C@@]2(C[C@H]1C#N)Oc1cc(F)cc(F)c1NC2=O] | 544 [M + Na]⁺ | ¹H NMR (400 MHz, 80° C., DMSO-d₆) δ 11.07 (br, 1H), 6.82- 7.21 (m, 2H), 6.51-6.81 (m, 1H), 4.81-5.58 (m, 2H), 4.27-4.59 (m, 1H), 3.88-4.25 (m, 2H), 3.51 (s, 3H), 2.92 (s, 3H), 2.53-2.82 (m, 2H), 1.39-1.78 (m, 3H), 0.96- 1.25 (m, 3H), 0.51-0.95 (m, 6H) |
| 71 | 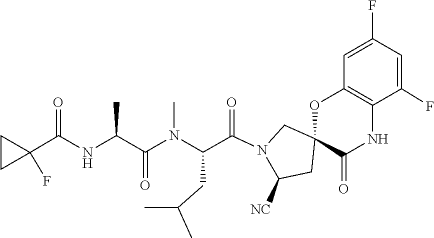 [SMILES: CC(C)C[C@@H](C(=O)N1C[C@@]2(C[C@H]1C#N)Oc1cc(F)cc(F)c1NC2=O)N(C)C(=O)[C@H](C)NC(=O)C1(F)CC1] | 572 [M + Na]⁺ | ¹H NMR (400 MHz, 80° C., DMSO-d₆) δ 11.18 (br, 1H), 7.70- 8.20 (m, 1H), 6.91-7.20 (m, 1H), 6.49-6.90 (m, 1H), 5.10-5.60 (m, 1H), 4.85-5.09 (m, 1H), 4.45- 4.84 (m, 1H), 3.65-4.44 (m, 2H), 2.88-3.00 (m, 3H), 2.60-2.87 (m, 2H), 1.55-1.90 (m, 3H), 1.20- 1.54 (m, 4H), 1.00-1.19 (m, 3H), 0.51-0.99 (m, 6H) |
| 72 | 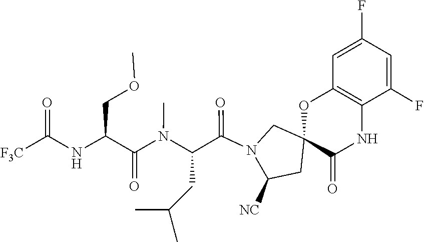 [SMILES: COC[C@H](NC(=O)C(F)(F)F)C(=O)N(C)[C@@H](CC(C)C)C(=O)N1C[C@@]2(C[C@H]1C#N)Oc1cc(F)cc(F)c1NC2=O] | 612 [M + Na]⁺ | ¹H NMR (400 MHz, 80º C, DMSO-d₆) δ 8.75-10.40 (m, 1H), 6.85-7.06 (m, 1H), 6.50-6.84 (m, 1H), 5.10-5.55 (m, 2H), 4.70- 5.09 (m, 1H), 3.90-4.20 (m, 2H), 3.44-3.52 (m, 2H), 3.15-3.18 (m, 3H), 2.92-2.98 (m, 3H), 2.69- 2.78 (m, 1H), 2.56-2.68 (m, 1H), 1.50-1.65 (m, 2H), 1.38-1.40 (m, 1H), 0.78-0.91 (m, 6H) |
| 73 | 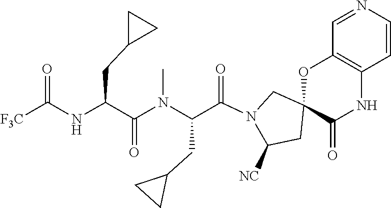 [SMILES: CN(C(=O)[C@H](CC1CC1)NC(=O)C(F)(F)F)[C@@H](CC1CC1)C(=O)N1C[C@@]2(C[C@H]1C#N)Oc1cnccc1NC2=O] | 563 [M + H]⁺ | ¹H NMR (400 MHz, 80º C, DMSO-d₆) δ 9.37 (br, 1H), 8.00- 8.25 (m, 2H), 6.85-7.00 (m, 1H), 5.15-5.40 (m, 1H), 4.86-5.14 (m, 1H), 4.60-4.85 (m, 1H), 4.00- 4.30 (m, 2H), 3.09 (s, 3H), 2.72- 2.90 (m, 1H), 2.55-2.71 (m, 1H), 1.60-1.80 (m, 2H), 1.20-1.59 (m, 2H), 0.50-0.80 (m, 2H), 0.20- 0.49 (m, 4H), −0.11-0.19 (m, 4H) |
| 74 | 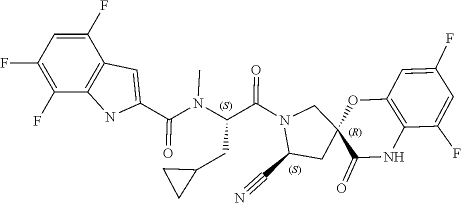 | 651 [M + Na + MeCN]⁺ | ¹H NMR (400 MHz, 80º C, DMSO-d₆) δ 10.35-12.80 (m, 2H), 6.98-7.20 (m, 1H), 6.81- 6.97 (m, 1H), 6.40-6.80 (m, 2H), 4.70-5.70 (m, 2H), 3.80-4.40 (m, 2H), 3.20 (s, 3H), 2.70-2.90 (m, 2H), 1.60-1.80 (m, 2H), 0.60- 0.90 (m, 1H), 0.30-0.55 (m, 2H), 0.05-0.28 (m, 2H) |
| 75 | 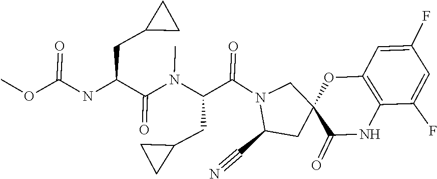 | 582 [M + Na]⁺ | ¹H NMR (400 MHz, 80° C., DMSO-d₆) δ 11.10 (br, 1H), 6.71- 7.26 (m, 2H), 6.39-6.69 (m, 1H), 5.01-5.37 (m, 1H), 4.70-4.99 (m, 1H), 4.25-4.50 (m, 1H), 3.80- 4.19 (m, 2H), 3.42 (s, 3H), 2.91 (s, 3H), 2.61-2.80 (m, 1H), 2.46- 2.60 (m, 1H), 1.45-1.80 (m, 2H), 1.00-1.44 (m, 2H), 0.42-0.70 (m, 2H), 0.15-0.41 (m, 4H), −0.03- 0.14 (m, 2H), −0.35 − −0.02 (m, 2H) |
| 76 | 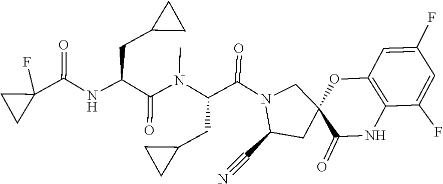 [SMILES: CN(C(=O)[C@H](CC1CC1)NC(=O)C1(F)CC1)[C@@H](CC1CC1)C(=O)N1C[C@@]2(C[C@H]1C#N)Oc1cc(F)cc(F)c1NC2=O] | 610 [M + Na]⁺ | ¹H NMR (400 MHz, 80° C., DMSO-d₆) δ 11.09 (br, 1H), 7.75 (s, 1H), 6.80-7.00 (m, 1H), 6.50- 6.70 (m, 1H), 5.01-5.31 (m, 1H), 4.79-5.00 (m, 1H), 4.60-4.78 (m, 1H), 3.80-4.28 (m, 2H), 2.98 (s, 3H), 2.66-2.79 (m, 1H), 2.49- 2.65 (m, 1H), 1.50-1.70 (m, 2H), 1.30-1.49 (m, 2H), 1.09-1.26 (m, 2H), 0.79-1.07 (m, 2H), 0.41- 0.70 (m, 2H), 0.13-0.40 (m, 4H), −0.06-0.11 (m, |
| | | | 2H), −0.39 − −0.04 (m, 2H). |
| 77 | 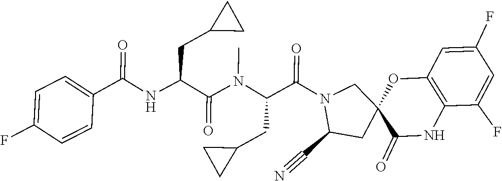 [SMILES: CN(C(=O)[C@H](CC1CC1)NC(=O)c1ccc(F)cc1)[C@@H](CC1CC1)C(=O)N1C[C@@]2(C[C@H]1C#N)Oc1cc(F)cc(F)c1NC2=O] | 646 [M + Na]⁺ | ¹H NMR (400 MHz, 80° C., DMSO-d₆) δ 11.12 (br, 1H), 8.25- 8.50 (m, 1H), 7.83-8.04 (m, 2H), 7.15-7.40 (m, 2H), 6.85-7.10 (m, 1H), 6.50-6.80 (m, 1H), 5.11- 5.40 (m, 1H), 4.70-5.10 (m, 2H), 3.75-4.40 (m, 2H), 3.10-3.35 (m, 3H), 2.55-2.90 (m, 2H), 1.40- 1.80 (m, 4H), 0.55-0.95 (m, 2H), 0.23-0.54 (m, 4H), −0.15-0.22 (m, 4H) |
| 78 | 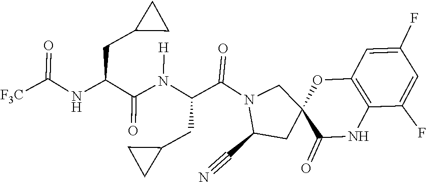 [SMILES: N#C[C@@H]1C[C@]2(CN1C(=O)[C@H](CC1CC1)NC(=O)[C@H](CC1CC1)NC(=O)C(F)(F)F)Oc1cc(F)cc(F)c1NC2=O] | 606 [M + Na]⁺ | ¹H NMR (400 MHz, 80° C. DMSO-d₆) δ 11.35 (br, 1H), 9.01 (br, 1H), 8.01-8.25 (m, 1H), 6.86- 6.96 (m, 1H), 6.61-6.72 (m, 1H), 4.72-4.91 (m, 1H), 4.31-4.45 (m, 2H), 4.12-4.22 (m, 1H), 3.91- 4.05 (m, 1H), 2.61-2.82 (m, 2H), 1.31-1.61 (m, 4H), 0.51-0.77(m, 2H), 0.15-0.41 (m, 4H), 0.03- 0.11(m, 4H) |
| 79 | 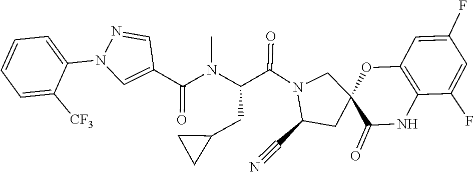 [SMILES: CN(C(=O)c1cnn(-c2ccccc2C(F)(F)F)c1)[C@@H](CC1CC1)C(=O)N1C[C@@]2(C[C@H]1C#N)Oc1cc(F)cc(F)c1NC2=O] | 629 [M + Na]⁺ | ¹H NMR (400 MHz, 80º C, DMSO-d₆) δ 11.06 (br, 1H), 8.28 (s, 1H), 7.89-8.05 (m, 2H), 7.80- 7.88 (m, 1H), 7.70-7.79 (m, 1H), 7.60-7.69 (m, 1H), 6.80-7.00 (m, 1H), 6.55-6.79 (m, 1H), 4.82- 5.40 (m, 2H), 4.15-4.58 (m, 1H), 3.80-4.14 (m, 1H), 3.10-3.30 (m, 3H), 2.65-2.90 (m, 2H), 1.50- 1.90 (m, 2H), 0.60-0.80 (m, 1H), 0.28-0.58 (m, 2H), 0.05-0.25 (m, 2H) |
| 83 | 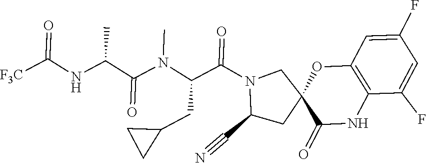 [SMILES: C[C@@H](NC(=O)C(F)(F)F)C(=O)N(C)[C@@H](CC1CC1)C(=O)N1C[C@@]2(C[C@H]1C#N)Oc1cc(F)cc(F)c1NC2=O] | 580 [M + Na]⁺ | ¹H NMR (400 MHz, 80° C., DMSO-d₆) δ 10.07 (br, 1H), 9.18 (br, 1H), 6.81-7.07 (m, 1H), 6.59- 6.74 (m, 1H), 5.09-5.34 (m, 1H), 4.87-5.08 (m, 1H), 4.55-4.86 (m, 1H), 3.75-4.30 (m, 2H), 2.99 (s, 3H), 2.52-2.83 (m, 2H), 1.52- 1.85 (m, 2H), 1.00-1.40 (m, 3H), 0.50-0.70 (m, 1H), 0.29-0.48 (m, 2H), 0.01-0.23 (m, 2H) |
| 84 | 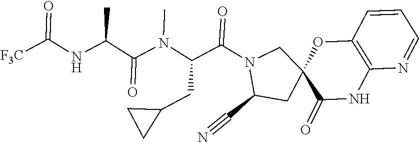 [SMILES: C[C@H](NC(=O)C(F)(F)F)C(=O)N(C)[C@@H](CC1CC1)C(=O)N1C[C@@]2(C[C@H]1C#N)Oc1cccnc1NC2=O] | 545 [M + Na]⁺ | ¹H NMR (400 MHz, 80° C., DMSO-d₆) δ 11.35 (br, 1H), 9.26 (br, 1H), 7.85-8.02 (m, 1H), 7.22-7.47 (m, 1H), 6.80-7.00 (m, 1H), 4.98-5.30 (m, 1H), 4.75- 4.97 (m, 1H), 4.45-4.73 (m, 1H), 3.80-4.20 (m, 2H), 2.88 (s, 3H), 2.50-2.80 (m, 2H), 1.40-1.70 (m, 2H), 0.80-1.20 (m, 3H), 0.41- |
| | | | 0.65 (m, 1H), 0.15-0.40 (m, |
| | | | 2H), −0.05 − 0.10 (m, 2H) |
| 85 | 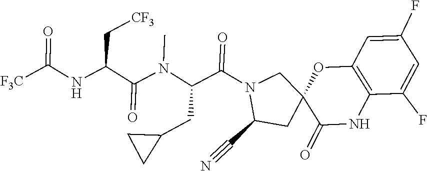 [SMILES: CN(C(=O)[C@H](CC(F)(F)F)NC(=O)C(F)(F)F)[C@@H](CC1CC1)C(=O)N1C[C@@]2(C[C@H]1C#N)Oc1cc(F)cc(F)c1NC2=O] | 648 [M + Na]⁺ | ¹H NMR (400 MHz, 80º C, DMSO-d₆) δ 9.20-11.80 (m, 2H), 6.88-7.05 (m, 1H), 6.61-6.84 (m, 1H), 4.79-5.26 (m, 3H), 3.92- 4.29 (m, 2H), 2.91-3.03 (m, 3H), 2.55-2.87 (m, 4H), 1.49-1.79 (m, 2H), 0.46-0.69 (m, 1H), 0.25- 0.45 (m, 2H), 0.02-0.18 (m, 2H) |
| 86 | 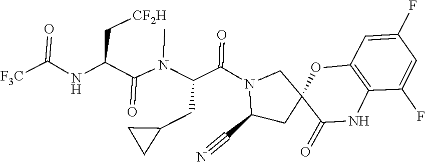 [SMILES: CN(C(=O)[C@H](CC(F)F)NC(=O)C(F)(F)F)[C@@H](CC1CC1)C(=O)N1C[C@@]2(C[C@H]1C#N)Oc1cc(F)cc(F)c1NC2=O] | 630 [M + Na]⁺ | ¹H NMR (400 MHz, 80º C, DMSO-d₆) δ 8.52-11.90 (m, 2H), 6.79-7.30 (m, 1H), 6.45-6.78 (m, 1H), 5.70-6.25 (m, 1H), 4.95- 5.35 (m, 1H), 4.55-4.94 (m, 2H), 3.75-4.30 (m, 2H), 2.92-3.01 (m, 3H), 2.60-2.85 (m, 2H), 1.95- 2.30 (m, 2H), 1.40-1.70 (m, 2H), 0.40-0.60 (m, 1H), 0.15-0.39 (m, 2H), −0.20-0.14 (m, 2H) |
| 87 | 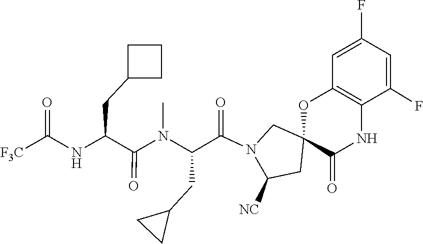 [SMILES: CN(C(=O)[C@H](CC1CCC1)NC(=O)C(F)(F)F)[C@@H](CC1CC1)C(=O)N1C[C@@]2(C[C@H]1C#N)Oc1cc(F)cc(F)c1NC2=O] | 634 [M + Na]⁺ | ¹H NMR (400 MHz, 80º C, DMSO-d₆) δ 11.10 (br, 1H), 9.25 (s, 1H), 6.86-7.00 (m, 1H), 6.50- 6.84 (m, 1H), 5.08-5.34 (m, 1H), 4.81-5.07 (m, 1H), 4.39-4.70 (m, 1H), 3.90-4.24 (m, 2H), 2.91- 2.98 (m, 3H), 2.72-2.84 (m, 1H), 2.52-2.68 (m, 1H), 2.10-2.30 (m, 1H), 1.75-1.95 (m, 2H), 1.50- 1.74 (m, 6H), 1.28-1.49 (m, 2H), 0.41-0.70 (m, 1H), 0.15-0.40 (m, 2H), −0.05-0.14 (m, 2H) |
| 88 | 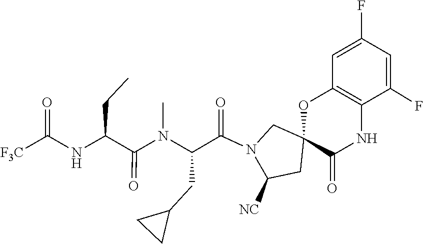 [SMILES: CC[C@H](NC(=O)C(F)(F)F)C(=O)N(C)[C@@H](CC1CC1)C(=O)N1C[C@@]2(C[C@H]1C#N)Oc1cc(F)cc(F)c1NC2=O] | 594 [M + Na]⁺ | ¹H NMR (400 MHz, 80° C., DMSO-d₆) δ 11.00 (br, 1H), 9.22 (s, 1H), 6.80 - 7.00 (m, 1H), 6.50- 6.79 (m, 1H), 5.00-5.25 (m, 1H), 4.75-4.98 (m, 1H), 4.38-4.60 (m, 1H), 3.80-4.20 (m, 2H), 2.81- 2.92 (m, 3H), 2.66-2.80 (m, 1H), 2.50-2.65 (m, 1H), 1.30-1.80 (m, 4H), 0.73 (t, J = 7.2 Hz, 3H), 0.40-0.60 (m, 1H), 0.18-0.39 (m, 2H), −0.05-0.19 (m, 2H). |
| 89 | 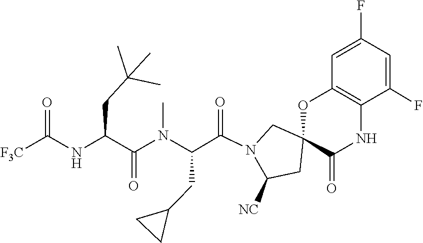 [SMILES: CN(C(=O)[C@H](CC(C)(C)C)NC(=O)C(F)(F)F)[C@@H](CC1CC1)C(=O)N1C[C@@]2(C[C@H]1C#N)Oc1cc(F)cc(F)c1NC2=O] | 636 [M + Na]⁺ | ¹H NMR (400 MHz, 80° C., DMSO-d₆) δ 11.05 (br, 1H), 9.41 (br, 1H), 6.80-7.15 (m, 1H), 6.50- 6.79 (m, 1H), 5.05-5.30 (m, 1H), 4.80-5.00 (m, 1H), 4.55-4.79 (m, 1H), 3.80-4.28 (m, 2H), 2.85- 3.00 (m, 3H), 2.61-2.84 (m, 1H), 2.45-2.60 (m, 1H), 1.51-1.91 (m, 3H), 1.32-1.50 (m, 1H), 0.50- 1.10 (m, 10H), 0.15-0.49 (m, 2H), −0.10-0.14 (m, 2H) |
| 90 | 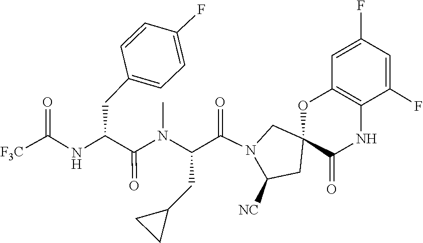 [SMILES: CN(C(=O)[C@@H](Cc1ccc(F)cc1)NC(=O)C(F)(F)F)[C@@H](CC1CC1)C(=O)N1C[C@@]2(C[C@H]1C#N)Oc1cc(F)cc(F)c1NC2=O] | 696 [M + Na]⁺ | ¹H NMR (400 MHz, 80° C., DMSO-d₆) δ 10.45-11.70 (m, 1H), 9.20-9.71 (m, 1H), 7.15- 7.55 (m, 2H), 6.91-7.14 (m, 2H), 6.79-6.90 (m, 1H), 6.40-6.78 (m, 1H), 4.71-5.28 (m, 3H), 3.95- 4.15 (m, 2H), 2.80-2.96 (m, 5H), 2.61-2.79 (m, 1H), 2.46-2.60 (m, 1H), 1.25- 1.80 (m, 2H), 0.39- 0.54 (m, 1H), 0.20-0.36 (m, 2H), −0.15-0.19 (m, 2H) |
| 91 | 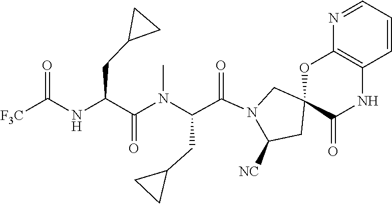 [SMILES: CN(C(=O)[C@H](CC1CC1)NC(=O)C(F)(F)F)[C@@H](CC1CC1)C(=O)N1C[C@@]2(C[C@H]1C#N)Oc1ncccc1NC2=O] | 563 [M + H]⁺ | ¹H NMR (400 MHz, DMSO-d₆) δ 11.25 (br, 1H), 9.69 (s, 1H), 7.81- 7.91 (m, 1H), 7.27-7.35 (m, 1H), 7.08-7.17 (m, 1H), 5.20-5.31 (m, 1H), 4.87-5.00 (m, 1H), 4.66- 4.79 (m, 1H), 4.10-4.20 (m, 1H), 3.99-4.08 (m, 1H), 2.89-3.03 (m, 3H), 2.74-2.86 (m, 1H), 2.60- 2.70 (m, 1H), 1.55-1.71 (m, 2H), 1.42-1.54 (m, 1H), 1.30-1.40 (m, 1H), 0.50-0.68 (m, 2H), 0.22- 0.41 (m, 4H), 0.03-0.14 (m, 2H), −0.04-0.02 (m, 1H), −0.15 − −0.05 (m, 1H). |
| 92 | 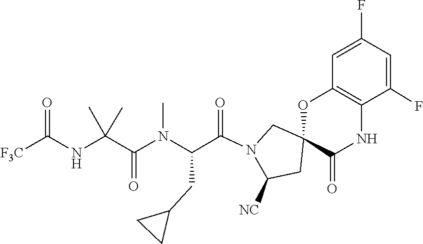 [SMILES: CN(C(=O)C(C)(C)NC(=O)C(F)(F)F)[C@@H](CC1CC1)C(=O)N1C[C@@]2(C[C@H]1C#N)Oc1cc(F)cc(F)c1NC2=O] | 594 [M + Na]⁺ | ¹H NMR (400 MHz, 80° C., DMSO-d₆) δ 11.00 (br, 0.4H), 9.42 (br, 1H), 6.75-7.10 (m, 1H), 6.50-6.74 (m, 1H), 4.70-5.20 (m, 2H), 4.10-4.40 (m, 1H), 3.80- 4.08 (m, 1H), 2.89 (s, 3H), 2.55- 2.80 (m, 2H), 1.51-1.70 (m, 1H), 1.40-1.50 (m, 1H), 1.20-1.39 (m, 6H), 0.45-0.68 (m, 1H), 0.20- 0.44 (m, 2H), −0.10-0.15 (m, 2H). |
| 94 | 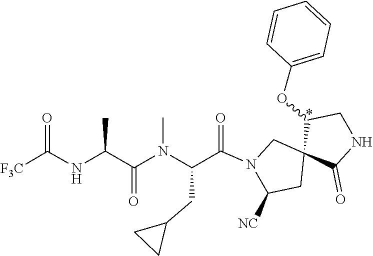 | 572 [M + Na]⁺ | ¹H NMR (400 MHz, 80° C., DMSO-d₆) δ 9.28 (br, 1H), 7.85 (s, 1H), 7.21-7.27 (m, 2H), 6.93- 6.97 (m, 1H), 6.80-6.89 (m, 2H), 5.27-5.29 (m, 1H), 4.82-4.89 (m, 1H), 4.54-4.79 (m, 2H), 4.07- 4.13 (m, 1H), 3.60-3.73 (m, 2H), 3.08-3.13 (m, 1H), 2.80-2.94 (m, 3H), 2.54-2.66 (m, 1H), 2.25- 2.34 (m, 1H), 1.53-1.79 (m, 2H), 1.03-1.33 (m, 3H), 0.54-0.70 (m, 1H), 0.28-0.46 (m, 2H), 0.01- 0.13 (m, 2H). |
| 95 | 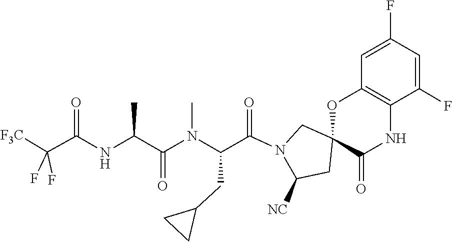 [SMILES: C[C@H](NC(=O)C(F)(F)C(F)(F)F)C(=O)N(C)[C@@H](CC1CC1)C(=O)N1C[C@@]2(C[C@H]1C#N)Oc1cc(F)cc(F)c1NC2=O] | 630 [M + Na]⁺ | ¹H NMR (400 MHz, 80° C., DMSO-d₆) δ 11.05 (br, 1H), 9.32 (br, 1H), 6.80-6.95 (m, 1H), 6.50- 6.78 (m, 1H), 5.00-5.30 (m, 1H), 4.75-4.99 (m, 1H), 4.55-4.74 (m, 1H), 3.80-4.20 (m, 2H), 2.80- 2.95 (m, 3H), 2.70-2.79 (m, 1H), 2.50-2.69 (m, 1H), 1.40-1.70 (m, 2H), 0.85-1.30 (m, 3H), 0.40- 0.60 (m, 1H), 0.15-0.38 (m, 2H), −0.10-0.12 (m, 2H). |
| 96 | 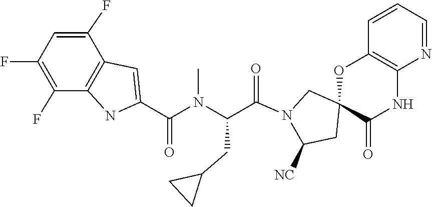 | 553 [M + H]⁺ | ¹H NMR (400 MHz, 80° C., DMSO-d₆) δ 11.75 (br, 1H), 7.70- 7.90 (m, 1H), 7.10-7.29 (m, 1H), 6.89-7.03 (m, 1H), 6.76-6.88 (m, 1H), 6.50-6.74 (m, 1H), 5.10- 5.40 (m, 1H), 4.80-5.09 (m, 1H), 3.83-4.40 (m, 2H), 3.14 (s, 3H), 2.55-2.80 (m, 2H), 1.60-1.85 (m, 2H), 0.58-0.80 (m, 1H), 0.20- 0.50 (m, 2H), 0.02-0.19 (m, 2H). |
| 97 | 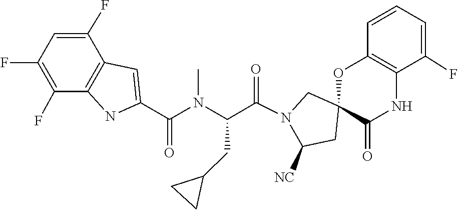 | 592 [M + Na]⁺ | ¹H NMR (400 MHz, DMSO-d₆) δ 11.90-12.90 (m, 1H), 10.90-11.60 (m, 1H), 7.00-7.20 (m, 1H), 6.90- 6.99 (m, 1H), 6.70-6.89 (m, 2H), 6.40-6.69 (m, 1H), 5.20-5.45 (m, 1H), 4.90-5.19 (m, 1H), 4.00- 4.20 (m, 2H), 3.20 (s, 3H), 2.60- 2.90 (m, 2H), 1.60-1.90 (m, 2H), 0.60-0.80 (m, 1H), 0.30-0.50 (m, 2H), 0.10-0.20 (m, 2H). |
| 98 | 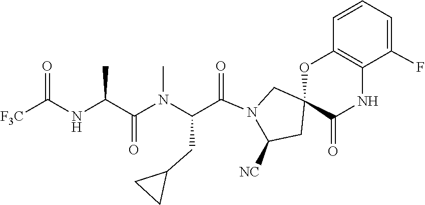 [SMILES: C[C@H](NC(=O)C(F)(F)F)C(=O)N(C)[C@@H](CC1CC1)C(=O)N1C[C@@]2(C[C@H]1C#N)Oc1cccc(F)c1NC2=O] | 562 [M + Na]⁺ | ¹H NMR (400 MHz, 80º C., DMSO-d₆) δ 10.70-11.20 (m, 1H), 9.00-9.50 (m, 1H), 6.80- 7.10 (m, 2H), 6.50-6.79 (m, 1H), 5.00-5.40 (m, 1H), 4.75-4.99 (m, 1H), 4.40-4.74 (m, 1H), 3.70- 4.20 (m, 2H), 2.90 (s, 3H), 2.50- 2.80 (m, 2H), 1.40-1.80 (m, 2H), 1.05 (s, 3H), 0.40-0.70 (m, 1H), 0.10-0.39 (m, 2H), −0.90-0.09 (m, 2H). |
| 100 | 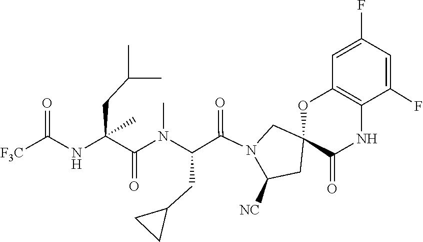 [SMILES: CC(C)C[C@](C)(NC(=O)C(F)(F)F)C(=O)N(C)[C@@H](CC1CC1)C(=O)N1C[C@@]2(C[C@H]1C#N)Oc1cc(F)cc(F)c1NC2=O] | 636 [M + Na]⁺ | ¹H NMR (400 MHz, 80° C., DMSO-d₆) δ 11.00 (br, 1H), 9.17 (s, 1H), 6.76-7.20 (m, 1H), 6.45- 6.74 (m, 1H), 4.60-5.30 (m, 2H), 4.15-4.50 (m, 1H), 3.75-4.10 (m, 1H), 2.82-3.00 (m, 3H), 2.60- 2.80 (m, 2H), 1.70-1.95 (m, 2H), 1.45-1.69 (m, 3H), 1.20-1.44 (m, 3H), 0.62-1.10 (m, 6H), 0.45- 0.61 (m, 1H), 0.20-0.44 (m, 2H), −0.10-0.19 (m, 2H) |
| 103 | 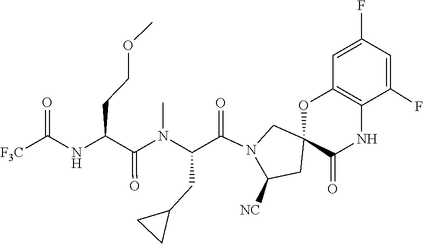 [SMILES: COCC[C@H](NC(=O)C(F)(F)F)C(=O)N(C)[C@@H](CC1CC1)C(=O)N1C[C@@]2(C[C@H]1C#N)Oc1cc(F)cc(F)c1NC2=O] | 624 [M + Na]⁺ | ¹H NMR (400 MHz, 80° C., DMSO-d₆) δ 11.0-11.4 (m, 1H), 9.20-10.1 (m, 1H), 7.01-7.21 (m, 1H), 6.71-6.91 (m, 1H), 5.11- 5.22 (m, 1H), 4.94 -5.02 (m, 1H), 4.72-4.85 (m, 1H), 3.91-4.22 (m, 2H), 3.15-3.28 (m, 2H), 3.11- 3.13 (m, 3H), 2.98-3.05 (m, 3H), 2.88-2.95 (m, 1H), 2.61-2.75 (m, 1H), 1.45-1.89 (m, 4H), 0.51- 0.71 (m,1H), 0.21-0.45 (m, 2H), 0.05-0.20 (m, 2H) |
| 104 | 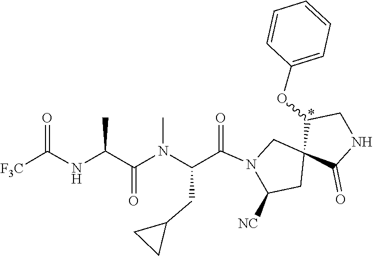 | 572 [M + Na]⁺ | ¹H NMR (400 MHz, 80° C., DMSO-d₆) δ 9.25 (br, 1H), 7.84 (br, 1H), 7.16-7.20 (m, 2H), 6.85- 6.91 (m, 1H), 6.72-6.80 (m, 2H), 4.90-5.10 (m, 1H), 4.56-4.73 (m, 3H), 3.96-4.05 (m, 1H), 3.61- 3.70 (m, 1H), 3.50-3.55 (m, 1H), 3.09-3.15 (m, 1H), 2.80-2.90 (m, 4H), 2.10-2.15 (m, 1H), 1.53- 1.70 (m, 2H), 0.95-1.24 (m, 3H), 0.60-0.70 (m, 1H), 0.21-0.29 (m, 2H), −0.10-0.05 (m, 2H). |
| | *wherein Compound 104 is the | | |
| | opposite stereoisomer of | | |
| | Compound 94 at the indicated | | |
| | carbon | | |
| 105 | 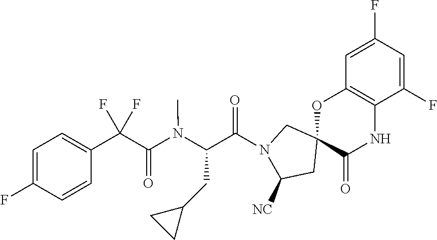 [SMILES: CN(C(=O)C(F)(F)c1ccc(F)cc1)[C@@H](CC1CC1)C(=O)N1C[C@@]2(C[C@H]1C#N)Oc1cc(F)cc(F)c1NC2=O] | 580 [M + NH₃ + H]⁺ | ¹H NMR (400 MHz, 80° C., DMSO-d₆) δ 11.00 (br, 1H), 7.30- 7.60 (m, 2H), 7.10-7.29 (m, 2H), 6.80-7.00 (m, 1H), 6.40-6.70 (m, 1H), 4.72-5.30 (m, 2H), 3.70- 4.20 (m, 2H), 2.75-2.95 (m, 3H), 2.50-2.74 (m, 2H), 1.20-1.80 (m, 2H), 0.39-0.69 (m, 1H), 0.15- 0.38 (m, 2H), −0.10-0.13 (m, 2H) |

### Example 47

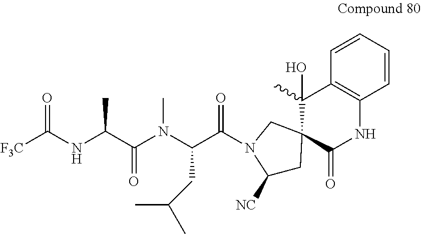

To a solution of 1-(t-butyl) 2,4-dimethyl (2S,4S)-4-((2-bromophenyl)(hydroxy)methyl)pyrrolidine-1,2,4-tricarboxylate (3.3 g, 6.99 mmol) in THF (50 mL) was added NaOH (1.40 g, 34.9 mmol, in 25 mL H₂O) stirred at rt. The mixture was stirred for 3 h at rt and then diluted with water (50 mL). The mixture was extracted with EA (3×100 mL). The organic layers were combined dried over anhydrous sodium sulfate, filtered and concentrated under reduced pressure to afford (2S,4S)-4-((2-bromophenyl)(hydroxy)methyl)-1-(t-butoxycarbonyl)pyrrolidine-2,4-dicarboxylic acid (2.6 g, crude) as a yellow solid. LC-MS (ESI, m/z): 344 [M−Boc+H]⁺.

To a solution of (2S,4S)-4-((2-bromophenyl)(hydroxy)methyl)-1-(t-butoxycarbonyl)pyrrolidine-2,4-dicarboxylic acid (2.6 g, 5.85 mmol) in THF (25 mL) were added 1-hydroxybenzotriazole (4.74 g, 35.1 mmol) and 1-(3-dimethylaminopropyl)-3-ethylcarbodiimide hydrochloride (5.61 g, 29.3 mmol) stirred at 0° C. After stirred for 1 h, NH₄OH (26 mL) was added. The mixture was stirred for 1 h at rt and then diluted with water (100 mL). The mixture was extracted with EA (3×200 mL). The organic layers were combined, dried over anhydrous sodium sulfate. The solids were removed by filtration and the filtrate was concentrated under reduced pressure. The crude product was purified by C18 column with CH₃CN:Water (0.05% NH₄HCO₃), (24%-31%). The fraction was concentrated under reduced pressure to provide t-butyl (2S,4S)-4-((2-bromophenyl)(hydroxy)methyl)-2,4-dicarbamoylpyrrolidine-1-carboxylate (2.0 g, crude) as a light yellow solid. LC-MS (ESI, m/z): 442 [M+H]⁺.

To a mixture of t-butyl (2S,4S)-4-((2-bromophenyl)(hydroxy)methyl)-2,4-dicarbamoylpyrrolidine-1-carboxylate (1.00 g, 2.27 mmol), (2-dicyclohexylphosphino-2′,4′,6′-triisopropylbiphenyl) (108 mg, 0.227 mmol) and XPhos Pd G3 (192 mg, 0.227 mmol) in 1,4-dioxane (10 mL) was added cesium carbonate (1.47 g, 4.54 mmol) at rt under nitrogen. The mixture was stirred for 1 h at 90° C. and the reaction was quenched with water (30 mL). The mixture was extracted with EA (3×100 mL). The organic layers were combined, washed with brine (3×60 mL), dried over anhydrous sodium sulfate. The solids were removed by filtration and the filtrate was concentrated under reduced pressure to afford the crude product. The crude product was chromatographed on a silica gel column with EA:PE (55%) to provide t-butyl (3S,5S)-5-carbamoyl-4′-hydroxy-2′-oxo-1′,4′-dihydro-2′H-spiro[pyrrolidine-3,3′-quinoline]-1-carboxylate (500 mg, 61%) as a yellow solid. ¹H NMR (400 MHz, DMSO-d₆) δ 10.18-10.36 (m, 1H), 7.09-7.47 (m, 3H), 6.80-7.07 (m, 3H), 5.60-5.81 (m, 1H), 4.23-4.57 (m, 1H), 3.99-4.22 (m, 1H), 3.69-3.93 (m, 1H), 2.16-2.47 (m, 1H), 1.68-2.01 (m, 1H), 1.26-1.56 (m, 9H). LC-MS (ESI, m/z): 362 [M+H]⁺.

To a mixture of t-butyl (3S,5S)-5-carbamoyl-4′-hydroxy-2′-oxo-1′,4′-dihydro-2′H-spiro[pyrrolidine-3,3′-quinoline]-1-carboxylate (500 mg, 1.39 mmol) in DCM (15 mL) were added cadmium oxide (415 mg, 4.17 mmol) and pyridine (657 mg, 8.34 mmol). The mixture was stirred for 5 h at rt and the reaction was quenched with water (30 mL). The mixture was extracted with EA (3×100 mL). The organic layers were combined, washed with brine (2×100 mL), dried over magnesium sulfate anhydrous, filtered and concentrated under reduced pressure to afford the crude product. The crude product was purified by prep-HPLC (Column: Xselect CSH Prep OBD C18 Column 30×150 mm, 5 m; Mobile Phase A: Water (0.1% FA), Mobile Phase B: ACN; Flow rate: 60 mL/min; Gradient: 18 to 48% B in 8 min; Wave Length: 220 nm) to provide t-butyl (3S,5S)-5-carbamoyl-2′,4′-dioxo-1′,4′-dihydro-2′H-spiro[pyrrolidine-3,3′-quinoline]-1-carboxylate (250 mg, 50%) as a white solid. ¹H NMR (400 MHz, DMSO-d₆) δ 10.95 (s, 1H), 7.72-7.83 (m, 1H), 7.52-7.69 (m, 1H), 7.36-7.50 (m, 1H), 6.96-7.18 (m, 3H), 4.12-4.33 (m, 1H), 3.76-3.94 (m, 1H), 3.54-3.68 (m, 1H), 2.40-2.49 (m, 1H), 2.22-2.38 (m, 1H), 1.25-1.49 (m, 9H). LC-MS (ESI, m/z): 360 [M+H]⁺.

A mixture of t-butyl (3S,5S)-5-carbamoyl-2′,4′-dioxo-1′,4′-dihydro-2′H-spiro[pyrrolidine-3,3′-quinoline]-1-carboxylate (150 mg, 0.417 mmol) in THF (0.4 mL) was added methyl magnesium bromide (0.4 mL, 3 M in THF) at −78° C. The mixture was stirred for 2 h at rt and the reaction was quenched with water. The mixture was extracted with EA (3×30 mL). The organic layers were combined, washed with brine (2×20 mL), dried over magnesium sulfate anhydrous, filtered and concentrated under reduced pressure. The crude product was chromatographed on a silica gel column with EA:PE (66%) to provide t-butyl (3S,5S)-5-carbamoyl-4′-hydroxy-4′-methyl-2′-oxo-1′,4′-dihydro-2′H-spiro[pyrrolidine-3,3′-quinoline]-1-carboxylate (100 mg, 64%) as a white solid. ¹H NMR (400 MHz, DMSO-d₆) δ 10.11-10.32 (m, 1H), 7.36-7.59 (m, 1H), 6.79-7.24 (m, 5H), 5.62-5.85 (m, 1H), 3.96-4.25 (m, 1H), 3.35-3.88 (m, 2H), 1.67-2.46 (m, 2H), 1.17-1.49 (m, 12H). LC-MS (ESI, m/z): 376 [M+H]⁺.

A mixture of t-butyl (3S,5S)-5-carbamoyl-4′-hydroxy-4′-methyl-2′-oxo-1′,4′-dihydro-2′H-spiro[pyrrolidine-3,3′-quinoline]-1-carboxylate (100 mg, 0.267 mmol) in HCl (2 mL, 3 M) was stirred for 1 h at rt. The mixture was alkalized to pH=8 with NaOH (4 M) and concentrated under reduced pressure to afford (3S,5S)-4′-hydroxy-4′-methyl-2′-oxo-1′,4′-dihydro-2′H-spiro[pyrrolidine-3,3′-quinoline]-5-carboxamide (100 mg, crude) as a white solid. LC-MS (ESI, m/z): 276 [M+H]⁺.

To a stirred mixture of (3S,5S)-4′-hydroxy-4′-methyl-2′-oxo-1′,4′-dihydro-2′H-spiro[pyrrolidine-3,3′-quinoline]-5-carboxamide (100 mg, 0.362 mmol), N-methyl-N-((2,2,2-trifluoroacetyl)-L-alanyl)-L-leucine (116 mg, 0.373 mmol) and o-(7-azabenzotriazol-1-yl)-N,N,N′,N′-tetramethyluronium hexafluorophosphate (165 mg, 0.434 mmol) in DMF (2 mL) was added N-ethyl-N-isopropylpropan-2-amine (82.0 mg, 0.724 mmol). The mixture was stirred for 40 mins at −15° C. and the reaction was quenched with water (10 mL). The mixture was extracted with EA (3×30 mL). The organic layers were combined, washed with brine (2×15 mL), dried over anhydrous sodium sulfate, filtered and concentrated under reduced pressure to afford the crude product. The crude product was purified by TLC (Mobile phase: MeOH/DCM=1:9; Rf=0.4; detection: UV) to provide (3S,5S)-4′-hydroxy-4′-methyl-1-(N-methyl-N-((2,2,2-trifluoroacetyl)-L-alanyl)-L-leucyl)-2′-oxo-1′,4′-dihydro-2′H-spiro[pyrrolidine-3,3′-quinoline]-5-carboxamide (95.0 mg, crude) as a white solid. LC-MS (ESI, m/z): 570 [M+H]⁺.

To a mixture of (3S,5S)-4′-hydroxy-4′-methyl-1-(N-methyl-N-((2,2,2-trifluoroacetyl)-L-alanyl)-L-leucyl)-2′-oxo-1′,4′-dihydro-2′H-spiro[pyrrolidine-3,3′-quinoline]-5-carboxamide (95.0 mg, 0.167 mmol) in DCM (2 mL) was added Burgess reagent (318 mg, 1.34 mmol). The mixture was stirred for 1 h at rt and the reaction was quenched with water (5 mL). The mixture was extracted with EA (3×20 mL). The organic layers were combined, washed with brine (2×10 mL), dried over anhydrous sodium sulfate, filtered and concentrated under reduced pressure. The crude product was purified by prep-HPLC (Column: Xselect CSH Prep OBD C18 Column 30×150 mm, 5 m; Mobile Phase A: Water (0.1% FA), Mobile Phase B: ACN; Flow rate: 60 mL/min; Gradient: 23% B to 53% B in 8 min; Wave Length: 220 nm) to provide (S)—N—((S)-1-((3S,5S)-5-cyano-4′-hydroxy-4′-methyl-2′-oxo-1′,4′-dihydro-2′H-spiro[pyrrolidine-3,3′-quinolin]-1-yl)-4-methyl-1-oxopentan-2-yl)-N-methyl-2-(2,2,2-trifluoroacetamido)propanamide (33 mg, 35%) as a white solid. ¹H NMR (400 MHz, 80° C., DMSO-d₆) δ 10.00-10.42 (m, 1H), 9.38 (br, 1H), 7.37-7.55 (m, 1H), 7.10-7.30 (m, 1H), 6.98-7.09 (m, 1H), 6.61-6.95 (m, 1H), 5.50-5.94 (m, 1H), 4.62-5.49 (m, 3H), 3.41-4.29 (m, 2H), 2.80-3.00 (m, 3H), 2.52-2.79 (m, 1H), 1.48-1.94 (m, 3H), 1.00-1.47 (m, 7H), 0.54-0.99 (m, 6H). LC-MS (ESI, m/z): 552 [M+H]⁺.

### Example 48

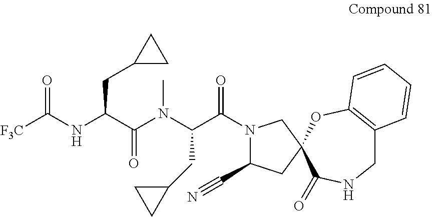 [SMILES: CN(C(=O)[C@H](CC1CC1)NC(=O)C(F)(F)F)[C@@H](CC1CC1)C(=O)N1C[C@@]2(C[C@H]1C#N)Oc1ccccc1CNC2=O]

To a mixture of 1-(t-butyl) 2-methyl (2S,4S)-4-hydroxy-4-(trichloromethyl)pyrrolidine-1,2-dicarboxylate (2.00 g, 5.51 mmol) and t-butyl (2-hydroxybenzyl)carbamate (1.85 g, 8.27 mmol) in acetone (50 mL) was added NaOH (1.10 g, 27.6 mmol) at 0° C. The mixture was stirred overnight at rt and the reaction was quenched with water (100 mL). The mixture was adjusted to pH=5 with HCl (1 M) and extracted with EA (3×100 mL). The organic layers were combined, washed with brine (2×100 mL) and dried over anhydrous sodium sulfate. The solvents were removed by filtration and the solvate was concentrated under reduced pressure to afford (2S,4R)-1-(t-butoxycarbonyl)-4-(2-(((t-butoxycarbonyl)amino)methyl)phenoxy)pyrrolidine-2,4-dicarboxylic acid (2.66 g, crude) as a brown oil. LC-MS (ESI, m/z): 503 [M+Na]⁺.

To a mixture of (2S,4R)-1-(t-butoxycarbonyl)-4-(2-(((t-butoxycarbonyl)amino)methyl)phenoxy)pyrrolidine-2,4-dicarboxylic acid (2.66 g, 5.54 mmol) and potassium carbonate (3.08 g, 22.1 mmol) in DMF (30 mL) was added iodomethane (1.57 g, 11.1 mmol) at 0° C. The mixture was stirred for 1 h at rt and the reaction was quenched with water (80 mL). The mixture was extracted with EA (3×80 mL). The organic layers were combined, washed with brine (2×80 mL) and dried over anhydrous sodium sulfate. The solids were removed by filtration and the filtrate was concentrated under reduced pressure. The crude product was purified by C18 column with CH₃CN:Water (0.05% TFA). The fraction was concentrated under reduced pressure to provide 1-(t-butyl) 2,4-dimethyl (2S,4R)-4-(2-(((t-butoxycarbonyl)amino)methyl)phenoxy)pyrrolidine-1,2,4-tricarboxylate (1.00 g, 33%). ¹H NMR (400 MHz, DMSO-d₆) δ 7.10-7.28 (m, 3H), 6.96-7.06 (m, 1H), 6.50-6.58 (m, 1H), 4.34-4.46 (m, 1H), 4.05-4.16 (m, 2H), 3.95-4.01 (m, 1H), 3.78-3.88 (m, 1H), 3.61-3.77 (m, 6H), 2.78-2.89 (m, 1H), 2.51-2.56 (m, 1H), 1.26-1.45 (m, 18H). LC-MS (ESI, m/z): 531 [M+Na]⁺.

To a solution of 1-(t-butyl) 2,4-dimethyl (2S,4R)-4-(2-(((t-butoxycarbonyl)amino)methyl)phenoxy)pyrrolidine-1,2,4-tricarboxylate (500 mg, 0.983 mmol) in DCM (10 mL) was added TFA (3 mL). The mixture was stirred for 1 h at rt and concentrated under reduced pressure to afford dimethyl (2S,4R)-4-(2-(aminomethyl)phenoxy)pyrrolidine-2,4-dicarboxylate (303 mg, crude). LC-MS (ESI, m/z): 309 [M+H]⁺.

To a mixture of dimethyl (2S,4R)-4-(2-(aminomethyl)phenoxy)pyrrolidine-2,4-dicarboxylate (303 mg, 0.983 mmol) in MeOH (5 mL) was added N-ethyl-N-isopropylpropan-2-amine (381 mg, 2.95 mmol). The mixture was stirred for 2 h at rt and concentrated under reduced pressure to afford methyl (2R,5'S)-3-oxo-4,5-dihydro-3H-spiro[benzo[f][1,4]oxazepine-2,3′-pyrrolidine]-5′-carboxylate (272 mg, crude). LC-MS (ESI, m/z): 277 [M+H]⁺.

A mixture of methyl (2R,5'S)-3-oxo-4,5-dihydro-3H-spiro[benzo[f][1,4]oxazepine-2,3′-pyrrolidine]-5′-carboxylate (272 mg, 0.984 mmol) in ammonia (5 mL, 7 M in MeOH) was stirred overnight at 50° C. in a sealed vial. The mixture was concentrated under reduced pressure to afford (2R,5'S)-3-oxo-4,5-dihydro-3H-spiro[benzo[f][1,4]oxazepine-2,3′-pyrrolidine]-5′-carboxamide (257 mg, crude). LC-MS (ESI, m/z): 262 [M+H]⁺.

To a mixture of (2R,5'S)-3-oxo-4,5-dihydro-3H-spiro[benzo[f][1,4]oxazepine-2,3′-pyrrolidine]-5′-carboxamide (257 mg, 0.984 mmol) and di-t-butyl dicarbonate (258 mg, 1.18 mmol) in DCM (5 mL) was added triethylamine (199 mg, 1.97 mmol). The mixture was stirred for 2 h at rt and concentrated under reduced pressure. The crude product was chromatographed on a silica gel column with MeOH:DCM (6:94) to provide t-butyl (2R,5'S)-5′-carbamoyl-3-oxo-4,5-dihydro-3H-spiro[benzo[f][1,4]oxazepine-2,3′-pyrrolidine]-1′-carboxylate (105 mg, 28%) as an off-white solid. ¹H NMR (400 MHz, DMSO-d₆) δ 8.48-8.56 (m, 1H), 7.48-7.57 (m, 1H), 7.33-7.41 (m, 2H), 7.14-7.23 (m, 1H), 6.99-7.09 (m, 1H), 6.90-6.97 (m, 1H), 4.11-4.38 (m, 3H), 3.74-3.87 (m, 1H), 3.54-3.62 (m, 1H), 2.51-2.57 (m, 1H), 2.29-2.40 (m, 1H), 1.41 (s, 9H). LC-MS (ESI, m/z): 362 [M+H]⁺.

To a mixture of t-butyl (2R,5'S)-5′-carbamoyl-3-oxo-4,5-dihydro-3H-spiro[benzo[f][1,4]oxazepine-2,3′-pyrrolidine]-1′-carboxylate (105 mg, 0.291 mmol) in DCM (1 mL) was added TFA (0.3 mL). The mixture was stirred for 1 h at rt and concentrated under reduced pressure to afford (2R,5'S)-3-oxo-4,5-dihydro-3H-spiro[benzo[f][1,4]oxazepine-2,3′-pyrrolidine]-5′-carboxamide 2,2,2-trifluoroacetate (109 mg, crude). LC-MS (ESI, m/z): 262 [M+H]*.

To a mixture of (2R,5'S)-3-oxo-4,5-dihydro-3H-spiro[benzo[f][1,4]oxazepine-2,3′-pyrrolidine]-5′-carboxamide 2,2,2-trifluoroacetate (109 mg, 0.290 mmol), (S)-3-cyclopropyl-2-((S)-3-cyclopropyl-N-methyl-2-(2,2,2-trifluoroacetamido)propanamido)propanoic acid (102 mg, 0.290 mmol) and HATU (133 mg, 0.348 mmol) in DMF (2 mL) was added N-ethyl-N-isopropylpropan-2-amine (113 mg, 0.870 mmol) at −15° C. The mixture was stirred for 1 hour at −15° C. and the reaction was quenched with water (10 mL). The mixture was extracted with EA (3×10 mL). The organic layers were combined, washed with brine (2×10 mL) and dried over anhydrous sodium sulfate. The solids were removed by filtration and the filtrate was concentrated under reduced pressure. The crude product was chromatographed on a silica gel column with MeOH:DCM (6:94) to provide (2R,5'S)-1′-(3-cyclopropyl-2-((S)-3-cyclopropyl-N-methyl-2-(2,2,2-trifluoroacetamido)propanamido)propanoyl)-3-oxo-4,5-dihydro-3H-spiro[benzo[f][1,4]oxazepine-2,3′-pyrrolidine]-5′-carboxamide (110 mg, 61%) as an off-white solid. ¹H NMR (400 MHz, DMSO-d₆) δ 9.59-9.74 (m, 1H), 8.47-8.65 (m, 1H), 7.44-7.50 (m, 1H), 7.32-7.49 (m, 2H), 7.17-7.24 (m, 1H), 7.01-7.07 (m, 1H), 6.95-7.00 (m, 1H), 5.23-5.35 (m, 1H), 4.70-4.84 (m, 1H), 4.41-4.49 (m, 1H), 4.30-4.38 (m, 1H), 4.00-4.14 (m, 2H), 3.88-3.99 (m, 1H), 2.90-3.00 (m, 3H), 2.29-2.43 (m, 1H), 2.13-2.24 (m, 1H), 1.57-1.71 (m, 2H), 1.39-1.56 (m, 2H), 0.51-0.71 (m, 2H), 0.26-0.42 (m, 2H), 0.11-0.24 (m, 2H), 0.00-0.10 (m, 2H), −0.29-−0.18 (m, 2H). LC-MS (ESI, m/z): 616 [M+Na]⁺.

To a mixture of (2R,5'S)-1′-(3-cyclopropyl-2-((S)-3-cyclopropyl-N-methyl-2-(2,2,2-trifluoroacetamido)propanamido)propanoyl)-3-oxo-4,5-dihydro-3H-spiro[benzo[f][1,4]oxazepine-2,3′-pyrrolidine]-5′-carboxamide (110 mg, 0.185 mmol) in DCM (2 mL) were added pyridine (73.0 mg, 0.925 mmol) and trifluoroacetic anhydride (78.0 mg, 0.370 mmol). The mixture was stirred for 1 h at rt and the reaction was quenched with water (5 mL). The mixture was extracted with DCM (3×5 mL). The organic layers were combined, washed with brine (2×5 mL) and dried over anhydrous sodium sulfate. The solids were removed by filtration and the filtrate was concentrated under reduced pressure to afford the crude product. The crude product was purified by prep-HPLC (Column: Xselect CSH Prep OBD C18 Column, 30×150 mm, 5 m; Mobile Phase A: Water (0.1% FA), Mobile Phase B: ACN; Flow rate: 60 mL/min; Gradient: 46% B to 61% B in 8 min; Wave Length: 254 nm/220 nm; RT(min): 6.27) to provide (S)—N—((S)-1-((2R,5'S)-5′-cyano-3-oxo-4,5-dihydro-3H-spiro[benzo[f][1,4]oxazepine-2,3′-pyrrolidin]-1′-yl)-3-cyclopropyl-1-oxopropan-2-yl)-3-cyclopropyl-N-methyl-2-(2,2,2-trifluoroacetamido)propanamide (39.5 mg, 36%) as an off-white solid. ¹H NMR (400 MHz, 80° C., DMSO-d₆) δ 9.41 (s, 1H), 8.51 (s, 1H), 7.25-7.50 (m, 2H), 7.00-7.24 (m, 2H), 5.20-5.50 (m, 1H), 4.95-5.19 (m, 1H), 4.62-4.79 (m, 1H), 4.20-4.50 (m, 2H), 3.80-4.19 (m, 2H), 3.04 (s, 3H), 2.70-2.90 (m, 1H), 2.55-2.69 (m, 1H), 1.35-1.95 (m, 4H), 0.55-0.80 (m, 2H), 0.30-0.54 (m, 2H), 0.20-0.29 (m, 2H), 0.02-0.19 (m, 2H), −0.30-−0.02 (m, 2H). LC-MS (ESI, m/z): 598 [M+Na]⁺.

### Example 49

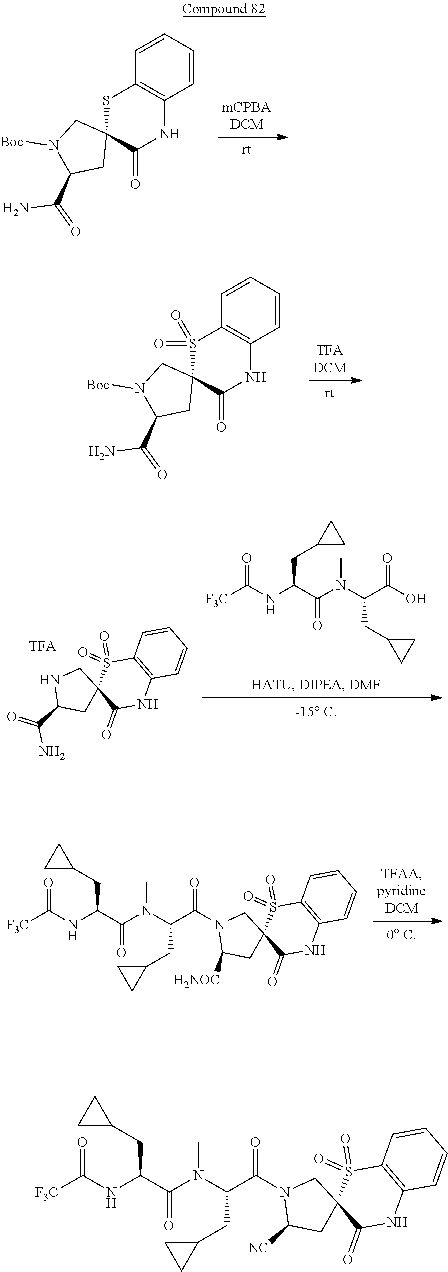 [SMILES: CC(C)(C)OC(=O)N1C[C@@]2(C[C@H]1C(N)=O)Sc1ccccc1NC2=O ; CC(C)(C)OC(=O)N1C[C@]2(C[C@H]1C(N)=O)C(=O)Nc1ccccc1S2(=O)=O ; NC(=O)[C@@H]1C[C@@]2(CN1)C(=O)Nc1ccccc1S2(=O)=O ; CN(C(=O)[C@H](CC1CC1)NC(=O)C(F)(F)F)[C@@H](CC1CC1)C(=O)O ; CN(C(=O)[C@H](CC1CC1)NC(=O)C(F)(F)F)[C@@H](CC1CC1)C(=O)N1C[C@]2(C[C@H]1C(N)=O)C(=O)Nc1ccccc1S2(=O)=O ; CN(C(=O)[C@H](CC1CC1)NC(=O)C(F)(F)F)[C@@H](CC1CC1)C(=O)N1C[C@]2(C[C@H]1C#N)C(=O)Nc1ccccc1S2(=O)=O]

To a solution of t-butyl (2R,5'S)-5′-carbamoyl-3-oxo-3,4-dihydrospiro[benzo[b][1,4]thiazine-2,3′-pyrrolidine]-1′-carboxylate (250 mg, 0.688 mmol) in DCM (2.5 mL) was added mCPBA (351 mg, 2.04 mmol). The mixture was stirred at rt for 20 h, diluted with DCM (20 mL) and washed with NaHCO₃ (sat., aq., 10 mL). The phases were separated. The organic phase was washed with NaHCO₃ (sat., aq., 10 mL), dried over Na₂SO₄, filtered and concentrated under reduced pressure. The residue was purified by flash chromatography on silica gel using a gradient of MeOH (2 to 5%) in DCM to afford t-butyl (2R,5'S)-5′-carbamoyl-3-oxo-3,4-dihydrospiro[benzo[b][1,4]thiazine-2,3′-pyrrolidine]-1′-carboxylate 1,1-dioxide (240 mg, 89%) as an off-white solid.

To a solution of t-butyl (2R,5'S)-5′-carbamoyl-3-oxo-3,4-dihydrospiro[benzo[b][1,4]thiazine-2,3′-pyrrolidine]-1′-carboxylate 1,1-dioxide (130 mg, 0.329 mmol) in DCM (1.3 mL) cooled at 0° C. was added TFA (0.18 mL, 1.64 mmol). The mixture was stirred at rt for 4 h. The mixture was concentrated under reduced pressure and co-evaporated with Et₂O to afford quantitatively (2R,5'S)-3-oxo-3,4-dihydrospiro[benzo[b][1,4]thiazine-2,3′-pyrrolidine]-5′-carboxamide 1,1-dioxide TFA salt as a colorless oil.

To a solution of (S)-3-cyclopropyl-2-((S)-3-cyclopropyl-N-methyl-2-(2,2,2-trifluoroacetamido)propanamido)propanoic acid (100 mg, 0.285 mmol) and (2R,5'S)-3-oxo-3,4-dihydrospiro[benzo[b][1,4]thiazine-2,3′-pyrrolidine]-5′-carboxamide 1,1-dioxide TFA salt (92 mg, 0.313 mmol) in DMF (1 mL) cooled at −15° C. were added HATU (162 mg, 0.427 mmol) and DIPEA (0.149 mL, 0.855 mmol). The mixture was stirred at −15° C. for 30 min. The mixture was diluted with water (5 mL) and extracted with EA (3×10 mL). The organic phases were combined, washed with water and brine, dried over Na₂SO₄, filtered and concentrated under reduced pressure. The residue was purified by flash chromatography on silica gel using a gradient of MeOH (2 to 5%) in DCM to afford (2R,5'S)-1′-((S)-3-cyclopropyl-2-((S)-3-cyclopropyl-N-methyl-2-(2,2,2-trifluoroacetamido)propanamido)propanoyl)-3-oxo-3,4-dihydrospiro[benzo[b][1,4]thiazine-2,3′-pyrrolidine]-5′-carboxamide 1,1-dioxide (100 mg, 59%) as an off-white solid.

To a solution of (2R,5'S)-1′-((S)-3-cyclopropyl-2-((S)-3-cyclopropyl-N-methyl-2-(2,2,2-trifluoroacetamido)propanamido)propanoyl)-3-oxo-3,4-dihydrospiro[benzo[b][1,4]thiazine-2,3′-pyrrolidine]-5′-carboxamide 1,1-dioxide (100 mg, 0.159 mmol) in DCM (1 mL) cooled at 0° C. were added pyridine (0.027 mL, 0.349 mmol) and TFAA (0.033 mL, 0.239 mmol). The mixture was stirred at 0° C. for 1 h. The mixture was diluted with water (3 mL) and extracted with DCM (3×10 mL). The organic phases were combined, washed with brine (2×5 mL), dried over Na₂SO₄, filtered and concentrated under reduced pressure. The residue was purified by prep-HPLC (Column: Daisogel-C18, 25×150 mm, 10 m; Mobile Phase A: 10 mM NH₄HCO₃ in water, Mobile Phase B: ACN; Flow rate: 22 mL/min; Gradient: 50% B to 65% B in 10 min) to afford (S)—N—((S)-1-((2R,5'S)-5′-cyano-1,1-dioxido-3-oxo-3,4-dihydrospiro[benzo[b][1,4]thiazine-2,3′-pyrrolidin]-1′-yl)-3-cyclopropyl-1-oxopropan-2-yl)-3-cyclopropyl-N-methyl-2-(2,2,2-trifluoroacetamido)propanamide (35 mg, 37%) as an off-white solid. ¹H NMR (500 MHz, 363K, DMSO-d₆) δ 11.33 (br. s., 1H), 9.32 (m, 1H), 7.83 (m, 1H), 7.75 (m, 1H), 7.36 (m, 1H), 7.31 (m, 1H), 5.30 (m, 1H), 5.06 (m, 1H), 4.81 (m, 1H), 4.23-4.37 (m, 2H), 2.90-3.00 (m, 3H), 2.66 (m, 1H), 2.42-2.53 (m, 1H), 1.53-1.77 (m, 4H), 0.72 (m, 1H), 0.61 (m, 1H), 0.32-0.48 (m, 4H), 0.17 (m, 1H), 0.02-0.11 (m, 3H). LC-MS (ESI, m/z): 610 [M+H]⁺.

### Example 50

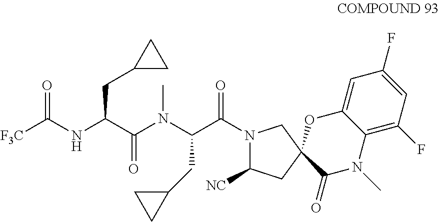 [SMILES: CN1C(=O)[C@]2(C[C@@H](C#N)N(C(=O)[C@H](CC3CC3)N(C)C(=O)[C@H](CC3CC3)NC(=O)C(F)(F)F)C2)Oc2cc(F)cc(F)c21]

To a solution of 1-(t-butyl) 2-methyl (2S,4S)-4-hydroxy-4-(trichloromethyl)pyrrolidine-1,2-dicarboxylate (4.0 g, 11.0 mmol) in acetone (40 mL) cooled to 0° C. were added 3,5-difluoro-2-nitrophenol (2.6 g, 22.1 mmol) and NaOH (2.6 g, 66.2 mmol). The mixture was stirred at rt for 16 h. The mixture was diluted with sat. NH₄C1, acidified with 1N HCl until pH 1 and extracted with EA (2×50 mL). The organic phases were combined, dried over Na₂SO₄, filtered and concentrated under reduced pressure. The residue was purified by flash chromatography on C18 column using a gradient of ACN (20 to 30%) in 0.1% FA in water to afford (2S,4R)-1-(t-butoxycarbonyl)-4-(3,5-difluoro-2-nitrophenoxy)pyrrolidine-2,4-dicarboxylic acid (2.5 g, 52%) as a yellow solid.

To a mixture of (2S,4R)-1-(t-butoxycarbonyl)-4-(3,5-difluoro-2-nitrophenoxy)pyrrolidine-2,4-dicarboxylic acid (2.5 g, 5.78 mmol) and K₂CO₃ (2.5 g, 18.5 mmol) in DMF (25 mL) cooled at 0° C. was added CH₃I (0.72 mL, 11.6 mmol). The mixture was stirred at rt for 1 h. The mixture was diluted with water (25 mL) and extracted with EA (3×30 mL). The organic phases were combined, washed with brine (2×20 mL), dried over Na₂SO₄, filtered and concentrated under reduced pressure. The residue was purified by flash chromatography on silica gel using a gradient of EA (15 to 20%) in PE to afford 1-(t-butyl) 2,4-dimethyl (2S,4R)-4-(3,5-difluoro-2-nitrophenoxy)pyrrolidine-1,2,4-tricarboxylate (1.0 g, 37%) as an off-white solid.

To a solution of 1-(t-butyl) 2,4-dimethyl (2S,4R)-4-(3,5-difluoro-2-nitrophenoxy)pyrrolidine-1,2,4-tricarboxylate (1 g, 2.17 mmol) in EtOH (20 mL) and water (4 mL) were added Fe (606 mg, 10.9 mmol) and NH₄Cl (601 mg, 10.9 mmol). The mixture was heated to 100° C. for 16 h. After cooling to rt, the mixture was filtered through celite and the solids were washed with EA (25 mL). The filtrate was dried over Na₂SO₄, filtered and concentrated under reduced pressure. The residue was purified by flash chromatography on silica gel using a gradient of EA (20 to 30%) in hexane to afford 1′-(tert-butyl) 5′-methyl (2R,5'S)-5,7-difluoro-3-oxo-3,4-dihydrospiro[benzo[b][1,4]oxazine-2,3′-pyrrolidine]-1′,5′-dicarboxylate (600 mg, 69%) as an off-white solid.

To a solution of 1′-(tert-butyl) 5′-methyl (2R,5'S)-5,7-difluoro-3-oxo-3,4-dihydrospiro[benzo[b][1,4]oxazine-2,3′-pyrrolidine]-1′,5′-dicarboxylate (600 mg, 1.51 mmol) in DMF (12 mL) cooled at 0° C. was added NaH (90 mg, 2.26 mmol). After 10 min at 0° C., MeI (0.46 mL, 7.53 mmol) was added and the mixture was stirred at rt for 3 h. The mixture was poured into ice-water (20 mL) and extracted with Et₂O (2×25 mL). The organic phases were combined, dried over Na₂SO₄, filtered and concentrated under reduced pressure. The residue was purified by flash chromatography on silica gel using a gradient of EA (20 to 30%) in PE to afford 1′-(t-butyl) 5′-methyl (2R,5'S)-5,7-difluoro-4-methyl-3-oxo-3,4-dihydrospiro[benzo[b][1,4]oxazine-2,3′-pyrrolidine]-1′,5′-dicarboxylate (500 mg, 67%) as an off-white solid.

A solution of 1′-(t-butyl) 5′-methyl (2R,5'S)-5,7-difluoro-4-methyl-3-oxo-3,4-dihydrospiro[benzo[b][1,4]oxazine-2,3′-pyrrolidine]-1′,5′-dicarboxylate (500 mg, 1.21 mmol) in 7M NH₃ in methanol (10 mL) was heated at 50° C. in a sealed tube for 24 h. The mixture was concentrated under reduced pressure to afford t-butyl (2R,5'S)-5′-carbamoyl-5,7-difluoro-4-methyl-3-oxo-3,4-dihydrospiro[benzo[b][1,4]oxazine-2,3′-pyrrolidine]-1′-carboxylate (1.05 g, 81%) as an off-white solid.

To a solution of t-butyl (2R,5'S)-5′-carbamoyl-5,7-difluoro-4-methyl-3-oxo-3,4-dihydrospiro[benzo[b][1,4]oxazine-2,3′-pyrrolidine]-1′-carboxylate (150 mg, 0.377 mmol) in DCM (3 mL) was added 4M HCl in dioxane (0.47 mL, 1.88 mmol). The mixture was stirred at rt for 2 h. The mixture was concentrated under reduced pressure and co-evaporated with Et₂O to afford quantitatively (2R,5'S)-5,7-difluoro-4-methyl-3-oxo-3,4-dihydrospiro[benzo[b][1,4]oxazine-2,3′-pyrrolidine]-5′-carboxamide hydrochloride as a colorless oil.

To a solution of (S)-3-cyclopropyl-2-((S)-3-cyclopropyl-N-methyl-2-(2,2,2-trifluoroacetamido)propanamido)propanoic acid (150 mg, 0.428 mmol, 1.0 eq.) and (2R,5'S)-5,7-difluoro-4-methyl-3-oxo-3,4-dihydrospiro[benzo[b][1,4]oxazine-2,3′-pyrrolidine]-5′-carboxamide hydrochloride (127 mg, 0.428 mmol) in DMF (3 mL) cooled to −15° C. were added HATU (195 mg, 0.514 mmol) and DIPEA (0.220 mL, 1.28 mmol). The mixture was stirred at −15° C. for 1 h. The mixture was diluted with water (5 mL) and extracted with EA (3×10 mL). The organic phases were combined, washed with water and brine, dried over Na₂SO₄, filtered and concentrated under reduced pressure. The residue was purified by flash chromatography on silica gel using a gradient of MeOH (3 to 6%) in DCM to afford (2R,5'S)-1′-((S)-3-cyclopropyl-2-((S)-3-cyclopropyl-N-methyl-2-(2,2,2-trifluoroacetamido)propanamido)propanoyl)-5,7-difluoro-4-methyl-3-oxo-3,4-dihydrospiro[benzo[b][1,4]oxazine-2,3′-pyrrolidine]-5′-carboxamide (150 mg, 55%) as an off-white solid.

To a solution of (2R,5'S)-1′-((S)-3-cyclopropyl-2-((S)-3-cyclopropyl-N-methyl-2-(2,2,2-trifluoroacetamido)propanamido)propanoyl)-5,7-difluoro-4-methyl-3-oxo-3,4-dihydrospiro[benzo[b][1,4]oxazine-2,3′-pyrrolidine]-5′-carboxamide (150 mg, 0.238 mmol, 1.0 eq.) in DCM (3 mL) cooled to 0° C. were added pyridine (0.058 mL, 0.714 mmol, 3.0 eq.) and TFAA (0.066 mL, 0.476 mmol, 2.0 eq.). The mixture was allowed to warm to rt over 1 h. The mixture was diluted with water (3 mL) and extracted with DCM (3×10 mL). The organic phases were combined, washed with brine (2×5 mL), dried over Na₂SO₄, filtered and concentrated under reduced pressure. The residue was purified by prep-HPLC (Column: X-SELECT-C18, 19*250 mm, 5 m; Mobile Phase A: 10 mM NH₄HCO₃ in water, Mobile Phase B: ACN; Flow rate: 16 mL/min; Gradient: 40% B to 70% B in 10 min) to afford (S)—N—((S)-1-((2R,5'S)-5′-cyano-5,7-difluoro-4-methyl-3-oxo-3,4-dihydrospiro[benzo[b][1,4]oxazine-2,3′-pyrrolidin]-1′-yl)-3-cyclopropyl-1-oxopropan-2-yl)-3-cyclopropyl-N-methyl-2-(2,2,2-trifluoroacetamido)propanamide (65 mg, 44%) as an off-white solid. ¹H NMR (500 MHz, 363K, DMSO-d₆) δ 9.20 (br. s., 1H), 7.03 (m, 1H), 6.77 (m, 1H), 5.18 (m, 1H), 4.94 (m, 1H), 4.79 (m, 1H), 4.12 (m, 2H), 3.40 (m, 3H), 3.02 (s, 3H), 2.63-2.76 (m, 2H), 1.65 (m, 2H), 1.52-1.62 (m, 2H), 0.57-0.72 (m, 2H), 0.31-0.43 (m, 4H), 0.00-0.13 (m, 4H). LCMS (ESI, m/z): 610 [M−H]⁻.

### Example 51

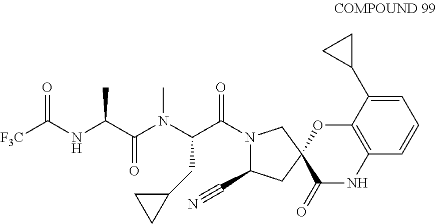 [SMILES: C[C@H](NC(=O)C(F)(F)F)C(=O)N(C)[C@@H](CC1CC1)C(=O)N1C[C@@]2(C[C@H]1C#N)Oc1c(cccc1C1CC1)NC2=O]

To a mixture of 1-(t-butyl) 2-methyl (2S,4S)-4-hydroxy-4-(trichloromethyl)pyrrolidine-1,2-dicarboxylate (3.00 g, 8.27 mmol) and 2,6-dibromophenol (4.17 g, 16.5 mmol) in acetone (30 mL) was added NaOH (1.99 g, 49.6 mmol) at 0° C. The mixture was stirred overnight at rt and the reaction was quenched with water (50 mL). The mixture was adjusted to pH=3˜4 with HCl (1 M). The mixture was extracted with EA (3×50 mL). The organic layers were combined and washed with sodium bicarbonate (2×50 mL). The aqueous layer was adjusted to pH=5 with HCl (3 M). The mixture was extracted with EA (3×100 mL). The organic layers were combined, dried over anhydrous sodium sulfate, filtered and concentrated under reduced pressure to afford the crude product. The crude product was purified by C18 column with CH₃CN:Water (0.05% TFA). The fraction was concentrated under reduced pressure to provide (2S,4R)-1-(t-butoxycarbonyl)-4-(2,6-dibromophenoxy)pyrrolidine-2,4-dicarboxylic acid (1.64 g, 38%) as a yellow solid. ¹H NMR (400 MHz, DMSO-d₆) δ 12.50-13.40 (m, 2H), 7.60-7.83 (m, 2H), 6.98-7.18 (m, 1H), 4.13-4.28 (m, 2H), 3.73-3.87 (m, 1H), 2.79-2.94 (m, 1H), 2.50-2.65 (m, 1H), 1.26-1.32 (m, 9H). LC-MS (ESI, m/z): 409[M−100+H]⁺.

To a mixture of (2S,4R)-1-(t-butoxycarbonyl)-4-(2,6-dibromophenoxy)pyrrolidine-2,4-dicarboxylic acid (1.64 g, 3.22 mmol, 1.0 eq.), HATU (5.39 g, 14.1 mmol) and ammonium chloride (3.45 g, 64.4 mmol) in DMF (25 mL) was added DIEA (6.24 g, 48.3 mmol) at 0° C. The mixture was stirred for 2 h at rt and the reaction was quenched with water (60 mL). The mixture was extracted with EA (3×60 mL). The organic layers were combined, washed with brine (2×50 mL), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide t-butyl (2S,4R)-2,4-dicarbamoyl-4-(2,6-dibromophenoxy)pyrrolidine-1-carboxylate (720 mg, 44%) as a red solid. ¹H NMR (400 MHz, DMSO-d₆) δ 7.78-7.90 (m, 1H), 7.66-7.77 (m, 2H), 7.57 (s, 1H), 7.32-7.45 (m, 1H), 7.01-7.14 (m, 1H), 6.88-6.99 (m, 1H), 3.91-4.09 (m, 2H), 3.66-3.83 (m, 1H), 2.55-2.68 (m, 1H), 2.42-2.49 (m, 1H), 1.21-1.46 (m, 9H). LC-MS (ESI, m/z): 506 [M+H]⁺.

To a mixture of t-butyl (2S,4R)-2,4-dicarbamoyl-4-(2,6-dibromophenoxy)pyrrolidine-1-carboxylate (720 mg, 1.42 mmol, 1.0 eq.), copper(I) iodide (162 mg, 0.852 mmol, 0.6 eq.) and cesium carbonate (927 mg, 2.84 mmol, 2.0 eq.) in THF (10 mL) was added N,N′-dimethyl-1,2-ethanediamine (225 mg, 2.55 mmol, 1.8 eq.). The mixture was stirred for 1 h at 70° C. The reaction was quenched with water (20 mL). The mixture was extracted with EA (3×25 mL). The organic layers were combined, dried over anhydrous sodium sulfate, filtered and concentrated under reduced pressure to afford the crude product. The crude product was purified by C18 column with CH₃CN:Water (0.05% TFA). The fraction was concentrated under reduced pressure to provide t-butyl (2R,5'S)-8-bromo-5′-carbamoyl-3-oxo-3,4-dihydrospiro[benzo[b][1,4]oxazine-2,3′-pyrrolidine]-1′-carboxylate (500 mg, 82%) as a yellow solid. ¹H NMR (400 MHz, DMSO-d₆) δ 11.14 (s, 1H), 7.51-7.61 (m, 1H), 7.22-7.31 (m, 1H), 6.90-7.15 (m, 3H), 4.28-4.38 (m, 1H), 3.68-3.79 (m, 1H), 3.49-3.64 (m, 1H), 2.44-2.49 (m, 1H), 2.30-2.42 (m, 1H), 1.30-1.47 (m, 9H). LC-MS (ESI, m/z): 370[M−56+H]⁺.

To a mixture of t-butyl (2R,5'S)-8-bromo-5′-carbamoyl-3-oxo-3,4-dihydrospiro[benzo[b][1,4]oxazine-2,3′-pyrrolidine]-1′-carboxylate (220 mg, 0.517 mmol, 1.0 eq.), cyclopropylboronic acid (267 mg, 3.10 mmol, 6.0 eq.), tricyclohexyl phosphine (44.0 mg, 0.155 mmol, 0.3 eq.), potassium phosphate (384 mg, 1.81 mmol, 3.5 eq.) and palladium(II) acetate (29.0 mg, 0.129 mmol, 0.25 eq.) in toluene (5 mL) was added water (0.3 mL) at rt. The reaction was stirred overnight at 100° C. under nitrogen and the reaction was quenched with water (10 mL). The mixture was extracted with EA (3×12 mL). The organic layers were combined, dried over anhydrous sodium sulfate, filtered and concentrated under reduced pressure to afford the crude product. The crude product was purified by C18 column with CH₃CN/Water (0.05% TFA). The fraction was concentrated under reduced pressure to provide t-butyl (2R,5'S)-5′-carbamoyl-8-cyclopropyl-3-oxo-3,4-dihydrospiro[benzo[b][1,4]oxazine-2,3′-pyrrolidine]-1′-carboxylate (80.0 mg, 40%) as a red solid. ¹H NMR (400 MHz, DMSO-d₆) δ 10.88 (s, 1H), 7.54 (s, 1H), 6.44-7.17 (m, 4H), 4.21-4.48 (m, 1H), 3.24-3.41 (m, 2H), 2.34-2.48 (m, 2H), 1.86-2.00 (m, 1H), 1.13-1.58 (m, 9H), 0.51-0.99 (m, 4H). LC-MS (ESI, m/z): 388 [M+H]*.

To a mixture of t-butyl (2R,5'S)-5′-carbamoyl-8-cyclopropyl-3-oxo-3,4-dihydrospiro[benzo[b][1,4]oxazine-2,3′-pyrrolidine]-1′-carboxylate (80.0 mg, 0.206 mmol, 1.0 eq.) in DCM (3 mL) was added trifluoroacetic acid (1 mL). The mixture was stirred for 1 h at rt and concentrated under reduced pressure to afford (2R,5'S)-8-cyclopropyl-3-oxo-3,4-dihydrospiro[benzo[b][1,4]oxazine-2,3′-pyrrolidine]-5′-carboxamide (60.0 mg, crude) as a yellow oil. LC-MS (ESI, m/z): 288 [M+H]⁺.

To a mixture of (2R,5'S)-8-cyclopropyl-3-oxo-3,4-dihydrospiro[benzo[b][1,4]oxazine-2,3′-pyrrolidine]-5′-carboxamide (60.0 mg, 0.209 mmol, 1.0 eq.), (S)-3-cyclopropyl-2-((S)—N-methyl-2-(2,2,2-trifluoroacetamido)propanamido)propanoic acid (65.0 mg, 0.209 mmol, 1.0 eq.) and HATU (95.0 mg, 0.251 mmol, 1.2 eq.) in DMF (1.5 mL) was added DIEA (81.0 mg, 0.627 mmol, 3.0 eq.) at −15° C. The mixture was stirred for 30 mins at −15° C. The mixture was purified by C18 column with CH₃CN:Water (0.05% TFA). The fraction was concentrated under reduced pressure to provide (2R,5'S)-8-cyclopropyl-1′-(3-cyclopropyl-2-((S)—N-methyl-2-(2,2,2-trifluoroacetamido)propanamido)propanoyl)-3-oxo-3,4-dihydrospiro[benzo[b][1,4]oxazine-2,3′-pyrrolidine]-5′-carboxamide (45.0 mg, 37%) as a red solid. ¹H NMR (400 MHz, DMSO-d6) δ 10.91-10.98 (m, 1H), 9.60-9.67 (m, 1H), 7.51 (s, 1H), 7.00 (s, 1H), 6.86-6.92 (m, 1H), 6.70-6.75 (m, 1H), 6.53-6.58 (m, 1H), 5.19-5.26 (m, 1H), 4.60-4.67 (m, 1H), 4.36-4.43 (m, 1H), 3.92-3.97 (m, 1H), 3.78-3.84 (m, 1H), 2.86-2.91 (m, 3H), 2.43-2.47 (m, 1H), 2.14-2.22 (m, 1H), 1.84-1.91 (m, 1H), 1.60-1.68 (m, 1H), 1.37-1.45 (m, 1H), 0.96-1.04 (m, 3H), 0.52-0.58 (m, 1H), 0.21-0.40 (m, 4H), −0.08-0.10 (m, 4H). LC-MS (ESI, m/z): 602 [M+Na]⁺.

To a mixture of (2R,5'S)-8-cyclopropyl-1′-(3-cyclopropyl-2-((S)—N-methyl-2-(2,2,2-trifluoroacetamido)propanamido)propanoyl)-3-oxo-3,4-dihydrospiro[benzo[b][1,4]oxazine-2,3′-pyrrolidine]-5′-carboxamide (45.0 mg, 0.078 mmol, 1.0 eq.) in DCM (1 mL) were added pyridine (31.0 mg, 0.390 mmol, 5.0 eq.) and trifluoroacetic anhydride (33.0 mg, 0.156 mmol, 2.0 eq.). The mixture was stirred for 1 h at rt and the reaction was quenched with water (5 mL). The mixture was extracted with DCM (3×5 mL). The organic layers were combined, dried over anhydrous sodium sulfate, filtered and concentrated under reduced pressure to afford the crude product. The crude product was purified by preparative-Achiral-SFC HPLC (Column: Torus 2-PIC OBD 30×150 mm, 5 um; Mobile Phase A: CO₂, Mobile Phase B: ACN: MeOH=4:1 (0.1% 2M NH₃-MEOH); Flow rate: 65 mL/min; Gradient: isocratic 18% B; Column Temperature(° C.): 35; Back Pressure(bar): 100; Wave Length: 220 nm; RT(min): 6.92; Sample Solvent: MEOH; Injection Volume: 1 mL) to provide (S)—N—((S)-1-((2R,5'S)-5′-cyano-8-cyclopropyl-3-oxo-3,4-dihydrospiro[benzo[b][1,4]oxazine-2,3′-pyrrolidin]-1′-yl)-3-cyclopropyl-1-oxopropan-2-yl)-N-methyl-2-(2,2,2-trifluoroacetamido)propanamide (5.5 mg, 12%) as a white solid. ¹H NMR (400 MHz, 80° C., DMSO-d₆) δ 10.74 (s, 1H), 9.27 (s, 1H), 6.79-6.95 (m, 1H), 6.60-6.77 (m, 1H), 6.41-6.55 (m, 1H), 5.05-5.30 (m, 1H), 4.75-4.98 (m, 1H), 4.50-4.70 (m, 1H), 3.70-4.10 (m, 2H), 2.86 (s, 3H), 2.65-2.80 (m, 1H), 2.50-2.64 (m, 1H), 1.75-1.90 (m, 1H), 1.55-1.70 (m, 1H), 1.40-1.54 (m, 1H), 0.89-1.15 (m, 3H), 0.68-0.89 (m, 2H), 0.40-0.67 (m, 3H), 0.15-0.39 (m, 2H), −0.05-0.14 (m, 2H). LC-MS (ESI, m/z): 584 [M+Na]⁺.

### Example 52

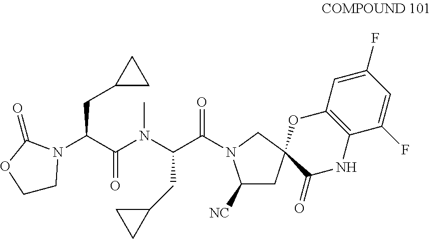 [SMILES: CN(C(=O)[C@H](CC1CC1)N1CCOC1=O)[C@@H](CC1CC1)C(=O)N1C[C@@]2(C[C@H]1C#N)Oc1cc(F)cc(F)c1NC2=O]

To a solution of (S)-3-cyclopropyl-2-(2-oxooxazolidin-3-yl)propanoic acid (67 mg, 0.337 mmol) and (2R,5'S)-1′-((S)-3-cyclopropyl-2-(methylamino)propanoyl)-5,7-difluoro-3-oxo-3,4-dihydrospiro[benzo[b][1,4]oxazine-2,3′-pyrrolidine]-5′-carboxamide (150 mg, 0.337 mmol) in DMF (2 mL) cooled to 0° C. were added HATU (191 mg, 0.505 mmol, 1.5 eq.) and DIPEA (0.176 mL, 1.01 mmol). The mixture was stirred at 0° C. for 30 min and then at rt for 1 h. The mixture was diluted with water (5 mL) and extracted with EA (3×10 mL). The organic phases were combined, washed with water and brine, dried over Na₂SO₄, filtered and concentrated under reduced pressure. The residue was purified by flash chromatography on silica gel using a gradient of MeOH (2 to 4%) in DCM to afford (2R,5'S)-1′-((S)-3-cyclopropyl-2-((S)-3-cyclopropyl-N-methyl-2-(2-oxooxazolidin-3-yl)propanamido)propanoyl)-5,7-difluoro-3-oxo-3,4-dihydrospiro[benzo[b][1,4]oxazine-2,3′-pyrrolidine]-5′-carboxamide (85 mg, 59%) as an off-white solid.

To a solution of (2R,5'S)-1′-((S)-3-cyclopropyl-2-((S)-3-cyclopropyl-N-methyl-2-(2-oxooxazolidin-3-yl)propanamido)propanoyl)-5,7-difluoro-3-oxo-3,4-dihydrospiro[benzo[b][1,4]oxazine-2,3′-pyrrolidine]-5′-carboxamide (75 mg, 0.127 mmol, 1.0 eq.) in DCM (1 mL) cooled to 0° C. were added pyridine (0.022 mL, 0.279 mmol, 2.2 eq.) and TFAA (0.019 mL, 0.140 mmol). The mixture was allowed to warm to rt over 1 h. The mixture was diluted with water (3 mL) and extracted with DCM (3×10 mL). The organic phases were combined, washed with brine (2×5 mL), dried over Na₂SO₄, filtered and concentrated under reduced pressure. The residue was purified by flash chromatography on C18 column using a gradient of ACN (50 to 60%) in 10 mM NH₄HCO₃ in water to afford (S)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-N-methyl-2-(2-oxooxazolidin-3-yl)propanamide (42 mg, 58%) as an off-white solid. ¹H NMR (500 MHz, 363K, DMSO-d₆) δ 9.60 (br. s., 1H), 6.90 (m, 1H), 6.66 (m, 1H), 5.16 (m, 1H), 4.94 (m, 1H), 4.71 (m, 1H), 4.24 (m, 2H), 4.14 (m, 1H), 4.03 (m, 1H), 3.45-3.62 (m, 2H), 3.02 (s, 3H), 2.66-2.82 (m, 2H), 1.53-1.74 (m, 3H), 1.49 (m, 1H), 0.59 (m, 2H), 0.29-0.49 (m, 4H), 0.06-0.15 (m, 2H), 0.00-0.05 (m, 2H). LCMS (ESI, m/z): 570 [M−H]⁻.

(S)-3-Cyclopropyl-2-(2-oxooxazolidin-3-yl)propanoic acid: To a solution of benzyl (S)-2-amino-3-cyclopropylpropanoate (400 mg, 1.82 mmol) in ethanol (4 mL) cooled to 0° C. was added 1M ethylene oxide in THF (1.82 mL, 1.82 mmol). The mixture was heated at 60° C. in a sealed tube for 4 h and then concentrated under reduced pressure. The residue was purified by flash chromatography on silica gel using a gradient of EA (70 to 100%) in PE to afford benzyl (S)-3-cyclopropyl-2-((2-hydroxyethyl)amino)propanoate (150 mg, 31%) as a colorless oil.

To a solution of benzyl (S)-3-cyclopropyl-2-((2-hydroxyethyl)amino)propanoate (150 mg, 0.570 mmol) in DCM (1.5 mL) cooled to 0° C. were added NEt₃ (0.095 mL, 0.684 mmol, 1.2 eq.) and triphosgene (169 mg, 0.570 mmol). The mixture was stirred at rt for 2 h. The mixture was diluted with water (10 mL) and extracted with DCM (3×10 mL). The organic phases were combined, dried over Na₂SO₄, filtered and concentrated under reduced pressure. The residue was purified by flash chromatography on silica gel using a gradient of EA (25 to 30%) in PE to afford benzyl (S)-3-cyclopropyl-2-(2-oxooxazolidin-3-yl)propanoate (60 mg, 36%) as a colorless oil.

To a solution of benzyl (S)-3-cyclopropyl-2-(2-oxooxazolidin-3-yl)propanoate (60 mg, 0.208 mmol, 1.0 eq.) in methanol (1 mL) was added 10% Pd/C (12 mg). The mixture was stirred at rt for 1 h under H₂ atmosphere. The mixture was filtered and the filtrate was concentrated under reduced pressure to get quantitatively (S)-3-cyclopropyl-2-(2-oxooxazolidin-3-yl)propanoic acid as a colorless oil.

### Example 53

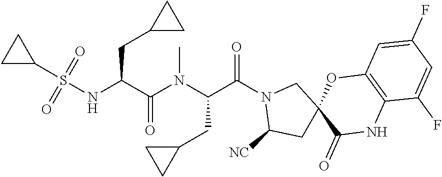 [SMILES: CN(C(=O)[C@H](CC1CC1)NS(=O)(=O)C1CC1)[C@@H](CC1CC1)C(=O)N1C[C@@]2(C[C@H]1C#N)Oc1cc(F)cc(F)c1NC2=O]

To a solution of (S)-2-(((benzyloxy)carbonyl)amino)-3-cyclopropylpropanoic acid (178 mg, 0.674 mmol) and (2R,5'S)-1′-((S)-3-cyclopropyl-2-(methylamino)propanoyl)-5,7-difluoro-3-oxo-3,4-dihydrospiro[benzo[b][1,4]oxazine-2,3′-pyrrolidine]-5′-carboxamide hydrochloride (300 mg, 0.674 mmol) in DMF (3 mL) cooled to 0° C. were added HATU (308 mg, 0.809 mmol) and DIPEA (0.294 mL, 1.69 mmol). The mixture was allowed to warm to rt over 1 h and then stirred at rt for 2 h. The mixture was poured into cold water (10 mL). After 5 min stirring, the solid was filtered and then recrystallized from DCM and heptane to afford benzyl ((S)-1-(((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-3-cyclopropyl-1-oxopropan-2-yl)carbamate (300 mg, 68%) as an off-white solid.

To a solution of benzyl ((S)-1-(((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-3-cyclopropyl-1-oxopropan-2-yl)carbamate (200 mg, 0.306 mmol) in DCM (6.9 mL) cooled to 0° C. were added NEt₃ (0.256 mL, 1.84 mmol) and TFAA (0.128 mL, 0.918 mmol). The mixture was stirred at 0° C. for 1.5 h. The mixture was washed with sat. NaHCO₃ (10 mL) and the phases were separated. The aqueous phase was extracted with DCM (2×20 mL). The organic phases were combined, washed with brine, dried over Na₂SO₄, filtered and concentrated under reduced pressure. The residue was purified by flash chromatography on silica gel using a gradient of MeOH (2 to 3%) in DCM to afford benzyl ((S)-1-(((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-3-cyclopropyl-1-oxopropan-2-yl)carbamate (150 mg, 77%) as an off-white solid.

A mixture of benzyl ((S)-1-(((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-3-cyclopropyl-1-oxopropan-2-yl)carbamate (150 mg, 0.236 mmol, 1.0 eq.) and 10% Pd/C (13 mg) in MeOH (3 mL) was stirred at rt for 4 h under H₂ atmosphere. The mixture was filtered through celite. The solids were washed with MeOH. The filtrate was concentrated under reduced pressure. The residue was purified by flash chromatography on silica gel using 5% MeOH in DCM as eluent to afford (S)-2-amino-N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-N-methylpropanamide (100 mg, 56%) as an off-white solid.

To a solution of (S)-2-amino-N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-N-methylpropanamide (100 mg, 0.199 mmol, 1.0 eq.) in DCM (1.5 mL) cooled to 0° C. were added DIPEA (0.104 mL, 0.598 mmol, 3.0 eq.) and cyclopropanesulfonyl chloride (0.024 mL, 0.239 mmol, 1.2 eq.). The mixture was stirred at rt for 3 h. The mixture was diluted with DCM (20 mL) and washed with sat. NaHCO₃ (5 mL). The phases were separated. The organic phase was washed with brine, dried over Na₂SO₄, filtered and concentrated under reduced pressure. The residue was purified by flash chromatography on C18 column using a gradient of ACN (40 to 50%) in 10 mM NH₄HCO₃ in water to afford (S)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-2-(cyclopropanesulfonamido)-3-cyclopropyl-N-methylpropanamide (79 mg, 65%) as a white solid. ¹H NMR (500 MHz, 363K, DMSO-d₆) δ 10.90 (br. s., 1H), 6.92 (m, 1H), 6.80 (br. s., 1H), 6.66 (m, 1H), 5.22 (m, 1H), 4.94 (m, 1H), 4.31 (m, 1H), 4.10-4.23 (m, 2H), 3.04 (s, 3H), 2.74-2.81 (m, 1H), 2.60-2.67 (m, 1H), 2.42 (m, 1H), 1.67 (m, 2H), 1.47-1.56 (m, 1H), 1.36-1.45 (m, 1H), 0.81-0.95 (m, 4H), 0.63-0.79 (m, 2H), 0.31-0.48 (m, 4H), 0.11 (m, 2H), 0.00-0.06 (m, 2H). LCMS (ESI, m/z): 604 [M−H]⁻.

### Example 54

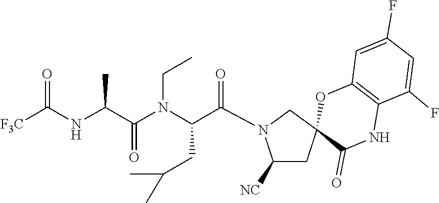 [SMILES: CCN(C(=O)[C@H](C)NC(=O)C(F)(F)F)[C@@H](CC(C)C)C(=O)N1C[C@@]2(C[C@H]1C#N)Oc1cc(F)cc(F)c1NC2=O]

Compound 106 was prepared similarly as described for Compound 37 using N-(t-butoxycarbonyl)-N-ethyl-L-leucine in place of (S)-2-((t-butoxycarbonyl)(methyl)amino)-3-cyclopropylpropanoic acid. ¹H NMR (500 MHz, 364K, DMSO-d₆) δ 11.00 (br. s., 1H), 9.30 (s, 1H), 6.93 (m, 1H), 6.64 (m, 1H), 5.18 (m, 1H), 4.95 (m, 1H), 4.66 (m, 1H), 3.87-4.17 (m, 2H), 3.54 (m, 1H), 3.35 (m, 1H), 2.65-2.85 (m, 2H), 1.74 (m, 1H), 1.42-1.55 (m, 2H), 1.09-1.23 (m, 6H), 0.80-0.92 (m, 6H). LCMS (ESI, m/z): 572 [M−H]⁻.

N-(t-butoxycarbonyl)-N-ethyl-L-leucine: To a solution of methyl L-leucinate hydrochloride (5 g, 27.6 mmol) in DCM (125 mL) and MeOH (5 mL) cooled to 0^{°} C were added NEt₃ (7.5 mL, 55.2 mmol), NaBH₃CN (1.37 g, 22.1 mmol) and acetaldehyde (1.85 mL, 33.1 mmol). The mixture was stirred at 0° C. for 2 h and then stirred at rt for 18 h. NaHCO₃ (5%, 50 mL) was added and the phases were separated. The aqueous phase was extracted with DCM (2×50 mL). The organic phases were combined, washed with brine (2×50 mL), dried over Na₂SO₄, filtered and concentrated under reduced pressure. The residue was purified by flash chromatography on silica gel using a gradient of MeOH (1 to 10%) in DCM to afford methyl ethyl-L-leucinate (2.5 g, 53%) as a colorless oil.

To a solution of methyl ethyl-L-leucinate (2.5 g, 14.2 mmol) in MeOH (25 mL) and water (20 mL) cooled to 0° C. were added Na₂CO₃ (1.5 g, 14.2 mmol) and (Boc)₂₀ (3.4 mL, 15.9 mmol). The mixture was stirred at rt for 16 h and then concentrated under reduced pressure. The residue was partitioned between water (30 mL) and EA (50 mL). The phases were separated. The aqueous phase was extracted with EA (50 mL). The organic phases were combined, dried over Na₂SO₄, filtered and concentrated under reduced pressure. The residue was purified by flash chromatography on silica gel using a gradient of EA (1 to 10%) in PE to afford methyl N-(t-butoxycarbonyl)-N-ethyl-L-leucinate (2.5 g, 64%) as a colourless liquid.

To a solution of methyl N-(t-butoxycarbonyl)-N-ethyl-L-leucinate (700 mg, 2.56 mmol) in THF (3.5 mL) and water (3.5 mL) cooled at 0° C. was added LiOH (161 mg, 3.84 mmol). The mixture was stirred at rt for 5 h and then partially concentrated under reduced pressure. The residue was acidified with 1N HCl until pH 2 and then extracted with EA (2×15 mL). The organic phases were combined, dried over Na₂SO₄, filtered and concentrated under reduced pressure to afford N-(t-butoxycarbonyl)-N-ethyl-L-leucine (600 mg, 90%) as a colourless oil.

### Example 55

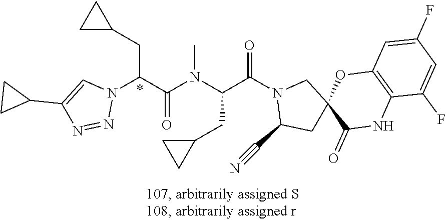 [SMILES: CN(C(=O)C(CC1CC1)n1cc(C2CC2)nn1)[C@@H](CC1CC1)C(=O)N1C[C@@]2(C[C@H]1C#N)Oc1cc(F)cc(F)c1NC2=O]

To a solution of methyl (S)-2-amino-3-cyclopropylpropanoate (1.0 g, 6.99 mmol) in MeOH (14 mL) were added a 0.4M CuSO₄ aqueous solution (1.7 mL, 0.699 mmol), NEt₃ (1.7 mL, 10.5 mmol) and a 0.4N TfN₃ solution in DCM (60 mL, 30.0 mmol). The mixture was stirred at rt for 16 h and then concentrated under reduced pressure. The residue was dissolved in MTBE (50 mL) and washed with aq. ammonia solution (30 mL). The phases were separated. The organic phase was washed with aq. ammonia solution (30 mL) and 1N NaHSO₄ (3×20 mL), dried over Na₂SO₄, filtered and concentrated under reduced pressure to afford benzyl methyl (S)-2-azido-3-cyclopropylpropanoate (850 mg, 72%) as a yellow oil.

To a solution of benzyl methyl (S)-2-azido-3-cyclopropylpropanoate (850 mg, 5.02 mmol) in THF (34 mL) and water (8.5 mL) cooled at 0° C. were added cyclopropyl acetylene (0.85 mL, 10.1 mmol), sodium ascorbate (1.0 g, 5.35 mmol, 1.05 eq.) and CuSO₄·5H₂O (375 mg, 1.50 mmol). The mixture was stirred at rt for 16 h and then concentrated under reduced pressure. The residue was dissolved in MTBE (50 mL) and washed with aq. ammonia solution (30 mL). The phases were separated. The organic phase was washed with aq. ammonia solution (30 mL) and brine (2×20 mL), dried over Na₂SO₄, filtered and concentrated under reduced pressure. The residue was purified by flash chromatography on silica gel using a gradient of EA (20 to 30%) in PE to afford methyl (S)-3-cyclopropyl-2-(4-cyclopropyl-1H-1,2,3-triazol-1-yl)propanoate (400 mg, 36%) as a colorless oil.

To a solution of methyl (S)-3-cyclopropyl-2-(4-cyclopropyl-1H-1,2,3-triazol-1-yl)propanoate (100 mg, 0.425 mmol, 1.0 eq.) in THF (1 mL) and water (1 mL) cooled at 0° C. was added LiOH (27 mg, 0.638 mmol, 1.5 eq.). The mixture was stirred at 0° C. for 4 h. The mixture was partially concentrated under reduced pressure. The residue was acidified with citric acid solution until pH 2 and extracted with DCM (20 mL). The phases were separated. The aqueous phase was extracted with DCM (20 mL). The organic phases were combined, dried over Na₂SO₄, filtered and concentrated under reduced pressure to get 3-cyclopropyl-2-(4-cyclopropyl-1H-1,2,3-triazol-1-yl)propanoic acid (75 mg, 82%) as a colorless oil.

To a solution of 3-cyclopropyl-2-(4-cyclopropyl-1H-1,2,3-triazol-1-yl)propanoic acid (140 mg, 0.633 mmol, 1.0 eq.) and (2R,5'S)-1′-((S)-3-cyclopropyl-2-(methylamino)propanoyl)-5,7-difluoro-3-oxo-3,4-dihydrospiro[benzo[b][1,4]oxazine-2,3′-pyrrolidine]-5′-carboxamide hydrochloride (281 mg, 0.633 mmol, 1.0 eq.) in DMF (2 mL) cooled at 0° C. were added HATU (360 mg, 0.949 mmol, 1.5 eq.) and DIPEA (0.329 mL, 1.89 mmol, 3.0 eq.). The mixture was stirred at 0° C. for 30 min and then at rt for 1 h. The mixture was diluted with water (5 mL) and extracted with EA (3×15 mL). The organic phases were combined, washed with water and brine, dried over Na₂SO₄, filtered and concentrated under reduced pressure. The residue was purified by flash chromatography on silica gel using a gradient of MeOH (3 to 5%) in DCM to afford (2R,5'S)-1′-((2S)-3-cyclopropyl-2-(3-cyclopropyl-2-(4-cyclopropyl-1H-1,2,3-triazol-1-yl)-N-methylpropanamido)propanoyl)-5,7-difluoro-3-oxo-3,4-dihydrospiro[benzo[b][1,4]oxazine-2,3′-pyrrolidine]-5′-carboxamide (300 mg, 75%) as an off-white solid.

To a solution of (2R,5'S)-1′-((2S)-3-cyclopropyl-2-(3-cyclopropyl-2-(4-cyclopropyl-1H-1,2,3-triazol-1-yl)-N-methylpropanamido)propanoyl)-5,7-difluoro-3-oxo-3,4-dihydrospiro[benzo[b][1,4]oxazine-2,3′-pyrrolidine]-5′-carboxamide (300 mg, 0.490 mmol, 1.0 eq.) in DCM (3 mL) cooled to 0° C. were added pyridine (0.086 mL, 1.08 mmol, 2.2 eq.) and TFAA (0.075 mL, 0.540 mmol, 1.1 eq.). The mixture was allowed to warm to rt over 1 h. The mixture was washed with water (10 mL). The phases were separated. The aqueous phase was extracted with DCM (2×20 mL). The organic phases were combined, washed with brine (2×5 mL), dried over Na₂SO₄, filtered and concentrated under reduced pressure. The residue was purified by prep-SFC using the following conditions: Column: Chiralcel OX—H, 4.6×25 cm, 5 m; Mobile Phase A: CO₂, Mobile Phase B: MeOH; Flow rate: 100 g/min; Elution condition: isocratic 20% B; Column Temperature: 30° C.; Back Pressure: 100 bar. Purification resulted in (R)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-2-(4-cyclopropyl-1H-1,2,3-triazol-1-yl)-N-methylpropanamide (85 mg, 30%, Compound 107) and (S)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-2-(4-cyclopropyl-1H-1,2,3-triazol-1-yl)-N-methylpropanamide (55 mg, 20%, Compound 108).

Compound 107: ¹H NMR (500 MHz, 363K, DMSO-d₆) δ 10.90 (br. s., 1H), 7.67 (s, 1H), 6.90 (m, 1H), 6.61 (m, 1H), 5.71 (m, 1H), 5.10 (m, 1H), 4.93 (m, 1H), 4.00-4.13 (m, 2H), 3.06 (s, 3H), 2.73 (m, 1H), 2.62 (m, 1H), 1.90 (m, 2H), 1.82 (m, 1H), 1.69 (m, 2H), 0.82 (m, 2H), 0.59-0.72 (m, 3H), 0.52 (m, 1H), 0.25-0.47 (m, 4H), 0.14-0.16 (m, 3H), −0.03 (m, 1H). LCMS (ESI, m/z): 594 [M+H]⁺. SFC: Chiralcel OX—H, 4.6×150 mm, 5 m, 30° C., co-Solvent: MeOH, hold 12 min at 20%, Rt: 5.54 min.

Compound 108: ¹H NMR (500 MHz, 363K, DMSO-d₆) δ 11.00 (br. s., 1H), 7.69 (s, 1H), 6.93 (m, 1H), 6.72 (m, 1H), 5.71 (m, 1H), 5.12 (m, 1H), 4.96 (m, 1H), 4.19 (m, 1H), 4.06 (m, 1H), 3.05 (s, 3H), 2.68-2.84 (m, 2H), 1.79-1.85 (m, 3H), 1.61 (m, 2H), 0.86 (m, 2H), 0.66 (m, 2H), 0.42-0.53 (m, 2H), 0.24-0.35 (m, 4H), 0.00-0.09 (m, 3H), −0.10 (m, 1H). LCMS (ESI, m/z): 594 [M+H]⁺. SFC: Chiralcel OX—H, 4.6×150 mm, 5 m, 30° C., co-Solvent: MeOH, hold 12 min at 20%, Rt: 6.66 min.

### Example 56

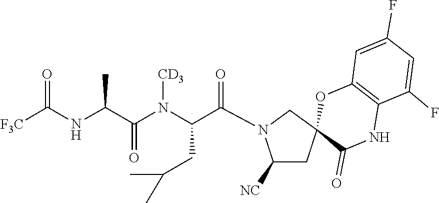 [SMILES: [2H]C([2H])([2H])N(C(=O)[C@H](C)NC(=O)C(F)(F)F)[C@@H](CC(C)C)C(=O)N1C[C@@]2(C[C@H]1C#N)Oc1cc(F)cc(F)c1NC2=O]

Compound 109 was prepared similarly as described for Compound 37 using N-(t-butoxycarbonyl)-N-(methyl-d3)-L-leucine in place of (S)-2-((t-butoxycarbonyl)(methyl)amino)-3-cyclopropylpropanoic acid. ¹H NMR (400 MHz, 363K, DMSO-d₆) δ 11.00 (br. s., 1H), 9.30 (br. s., 1H), 6.92 (m, 1H), 6.71 (m, 1H), 5.16 (m, 1H), 4.95 (m, 1H), 4.69 (m, 1H), 3.96-4.18 (m, 2H), 2.63-2.84 (m, 2H), 1.54-1.71 (m, 2H), 1.48 (m, 1H), 1.17 (m, 3H), 0.82-0.92 (m, 6H). LCMS (ESI, m/z): 563 [M+H]⁺.

N-(t-Butoxycarbonyl)-N-(methyl-d3)-L-leucine: To a solution of (t-butoxycarbonyl)-L-leucine (200 mg, 0.865 mmol) and CD₃I (0.57 mL, 8.65 mmol) in THF (10 mL) cooled at 0° C. was added portionwise NaH (207 mg, 8.65 mmol) over 2 h. The mixture was stirred at rt for 16 h in a sealed tube. The mixture was poured into ice/water (5 mL) and extracted with Et₂O (2×10 mL). The aqueous layer was cooled to 0° C., acidified with 1N HCl until pH 3 and extracted with EA (2×10 mL). The organic phases were combined, dried over Na₂SO₄, filtered and concentrated under reduced pressure to afford N-(t-butoxycarbonyl)-N-(methyl-d3)-L-leucine (200 mg, 93%) as a yellow oil.

### Example 57

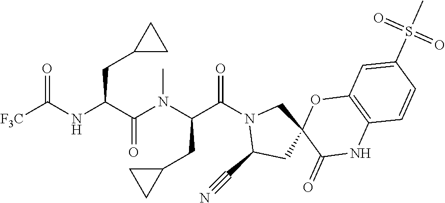 [SMILES: CN(C(=O)[C@H](CC1CC1)NC(=O)C(F)(F)F)[C@H](CC1CC1)C(=O)N1C[C@@]2(C[C@H]1C#N)Oc1cc(S(C)(=O)=O)ccc1NC2=O]

To a solution of 1-(t-butyl) 2-methyl (2S,4S)-4-hydroxy-4-(trichloromethyl)pyrrolidine-1,2-dicarboxylate (3.0 g, 8.31 mmol) in acetone (30 mL) cooled at 0° C. were added 5-bromo-2-iodophenol (3.7 g, 12.5 mmol) and NaOH (2.0 g, 49.9 mmol). The mixture was stirred at rt for 16 h. The mixture was diluted with sat. NH₄C1, acidified with 1N HCl until pH 1 and extracted with EA (2×30 mL). The organic phases were combined, dried over Na₂SO₄, filtered and concentrated under reduced pressure to afford (2S,4R)-1-(t-butoxycarbonyl)-4-(3,5-difluoro-2-nitrophenoxy)pyrrolidine-2,4-dicarboxylic acid (3.3 g, 73%) as a yellow solid.

To a mixture of (2S,4R)-1-(t-butoxycarbonyl)-4-(3,5-difluoro-2-nitrophenoxy)pyrrolidine-2,4-dicarboxylic acid (3.3 g, 5.96 mmol, 1.0 eq.) and K₂CO₃ (2.6 g, 19.1 mmol, 3.2 eq.) in DMF (30 mL) cooled at 0° C. was added methyl iodide (0.74 mL, 11.9 mmol, 2.0 eq.). The mixture was stirred at rt for 2 h. The mixture was diluted with water (25 mL) and extracted with EA (3×30 mL). The organic phases were combined, washed with brine (2×20 mL), dried over Na₂SO₄, filtered and concentrated under reduced pressure. The residue was purified by flash chromatography on silica gel using a gradient of EA (15 to 20%) in PE to afford 1-(t-butyl) 2,4-dimethyl (2S,4R)-4-(5-bromo-2-iodophenoxy)pyrrolidine-1,2,4-tricarboxylate (3.0 g, 87%) as an off-white solid.

A solution of 1-(t-butyl) 2,4-dimethyl (2S,4R)-4-(5-bromo-2-iodophenoxy)pyrrolidine-1,2,4-tricarboxylate (3.0 g, 5.15 mmol, 1.0 eq.) in 7M NH₃ in MeOH (60 mL) was heated at 50° C. in a sealed tube for 2 d and then concentrated under reduced pressure to obtain t-butyl (2S,4R)-4-(5-bromo-2-iodophenoxy)-2,4-dicarbamoylpyrrolidine-1-carboxylate (2.3 g, 85%) as an off-white solid.

To a solution of t-butyl (2S,4R)-4-(5-bromo-2-iodophenoxy)-2,4-dicarbamoylpyrrolidine-1-carboxylate (2.3 g, 4.15 mmol, 1.0 eq.) in THF (30 mL) were added Cs₂CO₃ (2.7 g, 8.30 mmol, 2.0 eq.), DMEDA (0.089 mL, 0.831 mmol, 0.2 eq.) and CuI (79 mg, 0.415 mmol, 0.2 eq.). The mixture was heated at 70° C. for 30 h. After cooling to rt, the mixture was filtered through celite. The filtrate was concentrated under reduced pressure. The residue was purified by flash chromatography on C18 column using a gradient of ACN (30 to 40%) in 0.1% FA in water to afford t-butyl (2R,5'S)-7-bromo-5′-carbamoyl-3-oxo-3,4-dihydrospiro[benzo[b][1,4]oxazine-2,3′-pyrrolidine]-1′-carboxylate (550 mg, 31%) as an off white solid.

To a solution of t-butyl (2R,5'S)-7-bromo-5′-carbamoyl-3-oxo-3,4-dihydrospiro[benzo[b][1,4]oxazine-2,3′-pyrrolidine]-1′-carboxylate (400 mg, 0.941 mmol, 1.0 eq.) in NMP (10 mL) were added CuI (1.07 g, 5.63 mmol, 6.1 eq.) and sodium methanesufinate (766 mg, 7.51 mmol, 8.1 eq.). The mixture was degassed for 5 min with N2 and heated at 150° C. for 20 h. After cooling to rt, the mixture was directly purified by flash chromatography on C18 column using a gradient of ACN (30 to 35%) in 0.1% FA in water to afford t-butyl (2R,5'S)-5′-carbamoyl-7-(methylsulfonyl)-3-oxo-3,4-dihydrospiro[benzo[b][1,4]oxazine-2,3′-pyrrolidine]-1′-carboxylate (180 mg, 45%) as an off-white solid.

To a solution of t-butyl (2R,5'S)-5′-carbamoyl-7-(methylsulfonyl)-3-oxo-3,4-dihydrospiro[benzo[b][1,4]oxazine-2,3′-pyrrolidine]-1′-carboxylate (180 mg, 0.422 mmol, 1.0 eq.) in DCM (4 mL) cooled at 0° C. was added 4M HCl in dioxane (0.52 mL, 2.11 mmol, 5.0 eq.). The mixture was stirred at rt for 2 h. The mixture was concentrated under reduced pressure and co-evaporated with Et₂O to afford quantitatively (2R,5'S)-7-(methylsulfonyl)-3-oxo-3,4-dihydrospiro[benzo[b][1,4]oxazine-2,3′-pyrrolidine]-5′-carboxamide hydrochloride as a colorless oil.

To a solution of (S)-3-cyclopropyl-2-((S)-3-cyclopropyl-N-methyl-2-(2,2,2-trifluoroacetamido)propanamido)propanoic acid (150 mg, 0.428 mmol, 1.0 eq.) and (2R,5'S)-7-(methylsulfonyl)-3-oxo-3,4-dihydrospiro[benzo[b][1,4]oxazine-2,3′-pyrrolidine]-5′-carboxamide hydrochloride (139 mg, 0.428 mmol, 1.0 eq.) in DMF (3 mL) cooled at −15° C. were added HATU (195 mg, 0.514 mmol, 1.2 eq.) and DIPEA (0.220 mL, 1.28 mmol, 3.0 eq.). The mixture was stirred at −15° C. for 1 h. The mixture was diluted with water (5 mL) and extracted with EA (3×10 mL). The organic phases were combined, washed with water and brine, dried over Na₂SO₄, filtered and concentrated under reduced pressure. The residue was purified by flash chromatography on C18 column using a gradient of ACN (30 to 35%) in 0.1% FA in water to afford (2R,5'S)-1′-((S)-3-cyclopropyl-2-((S)-3-cyclopropyl-N-methyl-2-(2,2,2-trifluoroacetamido)propanamido)propanoyl)-7-(methylsulfonyl)-3-oxo-3,4-dihydrospiro[benzo[b][1,4]oxazine-2,3′-pyrrolidine]-5′-carboxamide (130 mg, 46%) as an off-white solid.

To a solution of (2R,5'S)-1′-((S)-3-cyclopropyl-2-((S)-3-cyclopropyl-N-methyl-2-(2,2,2-trifluoroacetamido)propanamido)propanoyl)-7-(methylsulfonyl)-3-oxo-3,4-dihydrospiro[benzo[b][1,4]oxazine-2,3′-pyrrolidine]-5′-carboxamide (150 mg, 0.238 mmol, 1.0 eq.) in DCM (3 mL) cooled at 0° C. were added pyridine (0.058 mL, 0.714 mmol, 3.0 eq.) and TFAA (0.066 mL, 0.476 mmol, 2.0 eq.). The mixture was stirred at 0° C. for 30 min. The mixture was washed with water (3 mL). The phases were separated. The aqueous phase was extracted with DCM (3×10 mL). The organic phases were combined, washed with brine (2×5 mL), dried over Na₂SO₄, filtered and concentrated under reduced pressure. The residue was purified by flash chromatography on C18 column using a gradient of ACN (30 to 40%) in 0.1% FA in water and by prep-SFC (Column: Chiralpak IC, 3×25 cm, 5 m; Mobile Phase A: CO₂, Mobile Phase B: iPrOH; Flow rate: 90 g/min; Elution condition: isocratic 35% B; Column Temperature: 30° C.; Back Pressure: 100 bar) to afford (S)—N—((S)-1-((2R,5'S)-5′-cyano-7-(methylsulfonyl)-3-oxo-3,4-dihydrospiro[benzo[b][1,4]oxazine-2,3′-pyrrolidin]-1′-yl)-3-cyclopropyl-1-oxopropan-2-yl)-3-cyclopropyl-N-methyl-2-(2,2,2-trifluoroacetamido)propanamide (60 mg, 41%) as an off-white solid. ¹H NMR (500 MHz, 363K, DMSO-d₆) δ 11.30 (br. s., 1H), 9.10 (br. s., 1H), 7.60 (m, 1H), 7.43 (m, 1H), 7.17 (m, 1H), 5.25 (m, 1H), 5.03 (m, 1H), 4.83 (m, 1H), 4.11-4.24 (m, 2H), 3.13 (s, 3H), 3.07 (s, 3H), 2.81 (m, 1H), 2.64 (m, 1H), 1.89 (m, 2H), 1.54-1.66 (m, 2H), 0.68 (m, 2H), 0.30-0.46 (m, 4H), 0.12 (m, 2H), 0.06 (m, 1H), −0.02 (m, 1H). LCMS (ESI, m/z): 640 [M+H]⁺.

### Example 58

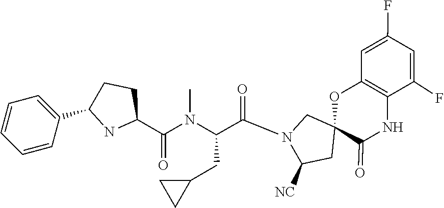

To a solution of (2S,5S)-1-((benzyloxy)carbonyl)-5-phenylpyrrolidine-2-carboxylic acid (88 mg, 0.270 mmol, 1.0 eq.) and (2R,5'S)-1′-((S)-3-cyclopropyl-2-(methylamino)propanoyl)-5,7-difluoro-3-oxo-3,4-dihydrospiro[benzo[b][1,4]oxazine-2,3′-pyrrolidine]-5′-carboxamide hydrochloride (120 mg, 0.270 mmol) in DMF (1.2 mL) cooled at 0° C. were added HATU (154 mg, 0.405 mmol) and DIPEA (0.141 mL, 0.810 mmol, 3.0 eq.). The mixture was stirred at rt for 1 h. The mixture was poured into ice/water (10 mL) and extracted with EA (2×20 mL). The organic phases were combined, dried over Na₂SO₄, filtered and concentrated under reduced pressure. The residue was purified by flash chromatography on silica gel using a gradient of EA (55 to 65%) in PE to afford benzyl (2S,5S)-2-(((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)carbamoyl)-5-phenylpyrrolidine-1-carboxylate (115 mg, 60%) as a colorless oil.

To a solution of benzyl (2S,5S)-2-(((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)carbamoyl)-5-phenylpyrrolidine-1-carboxylate (100 mg, 0.139 mmol, 1.0 eq.) in DCM (1 mL) cooled to 0° C. were added pyridine (0.024 mL, 0.305 mmol, 2.2 eq.) and TFAA (0.021 mL, 0.153 mmol, 1.1 eq.). The mixture was stirred at rt for 1 h. The mixture was washed with sat. NaHCO₃ (2 mL). The phases were separated. The aqueous phase was extracted with DCM (3×5 mL). The organic phases were combined, washed with brine, dried over Na₂SO₄, filtered and concentrated under reduced pressure. The residue was purified by flash chromatography on C18 column using a gradient of ACN (60 to 65%) in water to afford benzyl (2S,5S)-2-(((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)carbamoyl)-5-phenylpyrrolidine-1-carboxylate (85 mg, 88%) as an off-white solid.

To a solution of benzyl (2S,5S)-2-(((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)carbamoyl)-5-phenylpyrrolidine-1-carboxylate (75 mg, 0.107 mmol, 1.0 eq.) in MeOH (7.5 mL) was added 10% Pd/C (15 mg). The mixture was stirred at rt for 2 h under hydrogen atmosphere. The mixture was filtered through celite and the solids were washed with DCM (10 mL). The filtrate was concentrated under reduced pressure. The residue was purified by prep-HPLC (Column: Daisogel-C18, 25*150 mm, 10 m; Mobile Phase A: 10 mM NH₄HCO₃ in water, Mobile Phase B: ACN; Flow rate: 22 mL/min; Gradient: 30% B to 55% B in 8 min) to afford (2S,5S)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-N-methyl-5-phenylpyrrolidine-2-carboxamide (15 mg, 25%) as an off-white solid. ¹H NMR (500 MHz, 363K, DMSO-d₆) δ 10.85 (br. s., 1H), 7.31 (m, 2H), 7.25 (t, 2H), 7.15 (m, 1H), 6.92 (m, 1H), 6.69 (m, 1H), 5.17 (m, 1H), 4.97 (m, 1H), 4.23 (m, 1H), 4.14 (m, 1H), 3.96-4.10 (m, 2H), 2.92-3.00 (m, 4H), 2.73-2.85 (m, 2H), 1.99-2.13 (m, 2H), 1.70 (m, 1H), 1.52-1.67 (m, 3H), 0.65 (m, 1H), 0.40 (m, 2H), 0.06-0.15 (m, 2H). LCMS (ESI, m/z): 564 [M+H]⁺.

### Example 59

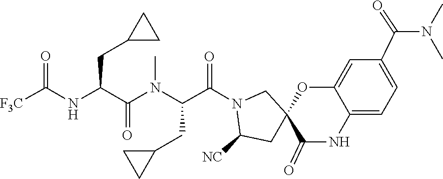 [SMILES: CN(C)C(=O)c1ccc2c(c1)O[C@@]1(C[C@@H](C#N)N(C(=O)[C@H](CC3CC3)N(C)C(=O)[C@H](CC3CC3)NC(=O)C(F)(F)F)C1)C(=O)N2]

To a solution of 1-(t-butyl) 2-methyl (2S,4S)-4-hydroxy-4-(trichloromethyl)pyrrolidine-1,2-dicarboxylate (3.0 g, 8.31 mmol, 1.0 eq.) in acetone (30 mL) cooled at 0° C. were added t-butyl (4-bromo-2-hydroxyphenyl)carbamate (4.7 g, 16.6 mmol, 2.0 eq.) and NaOH (2.0 g, 49.9 mmol, 6.0 eq.). The mixture was stirred at rt for 16 h. The mixture was diluted with sat. NH₄C1, acidified with 1N HCl until pH 3-5 and extracted with EA (3×50 mL). The organic phases were combined, dried over Na₂SO₄, filtered and concentrated under reduced pressure to afford (2S,4R)-4-(5-bromo-2-((t-butoxycarbonyl)amino)phenoxy)-1-(t-butoxycarbonyl)pyrrolidine-2,4-dicarboxylic acid (3.5 g,) as an oil.

To a mixture of (2S,4R)-4-(5-bromo-2-((t-butoxycarbonyl)amino)phenoxy)-1-(t-butoxycarbonyl)pyrrolidine-2,4-dicarboxylic acid (3.5 g, 6.43 mmol, 1.0 eq.) and K₂CO₃ (2.84 g, 20.6 mmol, 3.2 eq.) in DMF (35 mL) was added methyl iodide (0.80 mL, 12.9 mmol, 2.0 eq.). The mixture was stirred at rt for 2 h. The mixture was diluted with water (50 mL) and extracted with Et₂O (3×30 mL). The organic phases were combined, washed with brine (2×20 mL), dried over Na₂SO₄, filtered and concentrated under reduced pressure. The residue was purified by flash chromatography on silica gel using a gradient of EA (10 to 15%) in PE to afford 1-(t-butyl) 2,4-dimethyl (2S,4R)-4-(5-bromo-2-((t-butoxycarbonyl)amino)phenoxy)pyrrolidine-1,2,4-tricarboxylate (3.1 g, 65% over 2 steps) as an oil.

To a solution of 1-(t-butyl) 2,4-dimethyl (2S,4R)-4-(5-bromo-2-((t-butoxycarbonyl)amino)phenoxy)pyrrolidine-1,2,4-tricarboxylate (3.0 g, 5.24 mmol, 1.0 eq.) in DCM (52 mL) was added dropwise TFA (15 mL). The mixture was stirred at rt for 1 h and then concentrated under reduced pressure. The residue was neutralized with sat. NaHCO₃ until pH 7 and then extracted with EA (3×30 mL). The organic phases were combined, washed with brine (2×20 mL), dried over Na₂SO₄, filtered and concentrated under reduced pressure to afford methyl (2R,5'S)-7-bromo-3-oxo-3,4-dihydrospiro[benzo[b][1,4]oxazine-2,3′-pyrrolidine]-5′-carboxylate (1.3 g, 76%) as a brown solid.

A solution of methyl (2R,5'S)-7-bromo-3-oxo-3,4-dihydrospiro[benzo[b][1,4]oxazine-2,3′-pyrrolidine]-5′-carboxylate (1.2 g, 3.52 mmol, 1.0 eq.) in DCM (12 mL) were added Boc₂O (923 mg, 4.23 mmol, 1.2 eq) and NEt₃ (1 mL, 7.04 mmol, 2.0 eq.). The mixture was stirred at rt for 2 h and then concentrated under reduced pressure. The residue was purified by flash chromatography on silica gel using a gradient of EA (30 to 40%) in PE to afford 1′-(t-butyl) 5′-methyl (2R,5'S)-7-bromo-3-oxo-3,4-dihydrospiro[benzo[b][1,4]oxazine-2,3′-pyrrolidine]-1′,5′-dicarboxylate (1.0 g, 97%) as an off-white solid.

A solution of 1′-(t-butyl) 5′-methyl (2R,5'S)-7-bromo-3-oxo-3,4-dihydrospiro[benzo[b][1,4]oxazine-2,3′-pyrrolidine]-1′,5′-dicarboxylate (500 mg, 1.13 mmol, 1.0 eq). in dioxane (5 mL) was degassed under argon for 5 min and Pd(OAc)₂ (38 mg, 0.056 mmol, 0.05 eq.), Xantphos (33 mg, 0.056 mmol, 0.05 eq.), DMAP (206 mg, 1.69 mmol, 1.5 eq.), dicobalt octacarbonyl (97 mg, 0.28 mmol, 0.25 eq.) and 2N Me₂NH in THF (1.13 mL, 2.26 mmol, 2.0 eq.) were added. The mixture was heated at 90° C. for 30 min under microwave irradiation. After cooling to rt, Pd(OAc)₂ (38 mg, 0.056 mmol, 0.05 eq.), Xantphos (33 mg, 0.056 mmol, 0.05 eq.), DMAP (206 mg, 1.69 mmol, 1.5 eq.), dicobalt octacarbonyl (97 mg, 0.280 mmol, 0.25 eq.) and 2N Me₂NH in THF (1.13 mL, 2.26 mmol, 2.0 eq.) were added a second time. The mixture was filtered and the filtrate was concentrated under reduced pressure. The residue was purified by flash chromatography on silica gel using a gradient of EA (80 to 100%) in PE to afford 1′-(t-butyl) 5′-methyl (2R,5'S)-7-(dimethylcarbamoyl)-3-oxo-3,4-dihydrospiro[benzo[b][1,4]oxazine-2,3′-pyrrolidine]-1′,5′-dicarboxylate (235 mg, 54%) as an off-white solid.

A solution of 1′-(t-butyl) 5′-methyl (2R,5'S)-7-(dimethylcarbamoyl)-3-oxo-3,4-dihydrospiro[benzo[b][1,4]oxazine-2,3′-pyrrolidine]-1′,5′-dicarboxylate (230 mg, 1.0 eq.) in 7M NH₃ in MeOH (60 mL) was heated at 50° C. in a sealed tube for 24 h and then concentrated under reduced pressure to afford t-butyl (2R,5'S)-5′-carbamoyl-7-(dimethylcarbamoyl)-3-oxo-3,4-dihydrospiro[benzo[b][1,4]oxazine-2,3′-pyrrolidine]-1′-carboxylate (200 mg, 90%) as an off-white solid.

To a solution of t-butyl (2R,5'S)-5′-carbamoyl-7-(dimethylcarbamoyl)-3-oxo-3,4-dihydrospiro[benzo[b][1,4]oxazine-2,3′-pyrrolidine]-1′-carboxylate (180 mg, 0.449 mmol, 1.0 eq.) in DCM (1.8 mL) cooled at 0° C. was added 4M HCl in dioxane (0.5 mL, 2.15 mmol, 5.0 eq.). The mixture was stirred at rt for 2 h. The mixture was concentrated under reduced pressure and co-evaporated with Et₂O to afford quantitatively (2R,5'S)—N7,N7-dimethyl-3-oxo-3,4-dihydrospiro[benzo[b][1,4]oxazine-2,3′-pyrrolidine]-5′,7-dicarboxamide hydrochloride as an off-white solid.

To a solution of (S)-2-((S)-2-((t-butoxycarbonyl)amino)-3-cyclopropyl-N-methylpropanamido)-3-cyclopropylpropanoic acid (150 mg, 0.428 mmol, 1.0 eq.) and (2R,5'S)—N7,N7-dimethyl-3-oxo-3,4-dihydrospiro[benzo[b][1,4]oxazine-2,3′-pyrrolidine]-5′,7-dicarboxamide hydrochloride (150 mg, 0.428 mmol, 1.0 eq.) in DMF (1.5 mL) cooled at −15° C. were added HATU (243 mg, 0.642 mmol, 1.2 eq.) and DIPEA (0.220 mL, 1.28 mmol, 3.0 eq.). The mixture was stirred at −15° C. for 20 min. The mixture was diluted with water (5 mL) and extracted with EA (3×10 mL). The organic phases were combined, washed with water and brine, dried over Na₂SO₄, filtered and concentrated under reduced pressure. The residue was purified by flash chromatography on C18 column using a gradient of ACN (30 to 35%) in 0.1% FA in water to afford t-butyl ((S)-1-(((S)-1-((2R,5'S)-5′-carbamoyl-3-oxo-4,5-dihydro-3H-spiro[benzo[f][1,4]oxazepine-2,3′-pyrrolidin]-1′-yl)-3-cyclopropyl-1-oxopropan-2-yl)(methyl)amino)-3-cyclopropyl-1-oxopropan-2-yl)carbamate (90 mg, 32%) as an off-white solid.

To a solution of t-butyl ((S)-1-(((S)-1-((2R,5'S)-5′-carbamoyl-3-oxo-4,5-dihydro-3H-spiro[benzo[f][1,4]oxazepine-2,3′-pyrrolidin]-1′-yl)-3-cyclopropyl-1-oxopropan-2-yl)(methyl)amino)-3-cyclopropyl-1-oxopropan-2-yl)carbamate (85 mg, 0.131 mmol, 1.0 eq.) in DCM (0.8 mL) cooled at 0° C. were added pyridine (0.022 mL, 0.282 mmol, 2.2 eq.) and TFAA (0.020 mL, 0.143 mmol, 1.1 eq.). The mixture was stirred at rt for 1 h. The mixture was diluted with water (3 mL) and extracted with DCM (3×10 mL). The organic phases were combined, washed with brine (2×5 mL), dried over Na₂SO₄, filtered and concentrated under reduced pressure. The residue was purified by prep-HPLC (Column: X-SELECT-CSH—C18, 19*250 mm, 5 m; Mobile Phase A: 0.1% FA in water, Mobile Phase B: ACN; Flow rate: 16 mL/min; Gradient: 30% B to 70% B in 11 min) to afford (2R,5'S)-5′-cyano-1′-((S)-3-cyclopropyl-2-((S)-3-cyclopropyl-N-methyl-2-(2,2,2-trifluoroacetamido)propanamido)propanoyl)-N,N-dimethyl-3-oxo-3,4-dihydrospiro[benzo[b][1,4]oxazine-2,3′-pyrrolidine]-7-carboxamide (35 mg, 43%) as an off-white solid. ¹H NMR (500 MHz, 363K, DMSO-d₆) δ 10.95 (br. s., 1H), 9.25 (br. s., 1H), 7.07 (m, 1H), 6.98 (m, 1H), 6.93 (m, 1H), 5.20 (m, 1H), 4.96 (m, 1H), 4.80 (m, 1H), 4.16-4.24 (m, 1H), 4.10 (m, 1H), 3.01-3.06 (m, 3H), 2.94 (s, 6H), 2.73-2.81 (m, 1H), 2.57-2.61 (m, 1H), 1.49-1.74 (m, 4H), 0.57-0.72 (m, 2H), 0.26-0.44 (m, 4H), 0.10 (m, 2H), 0.02-0.07 (m, 1H), −0.01 (m, 1H). LCMS (ESI, m/z): 633 [M+H]⁺.

### Example 60

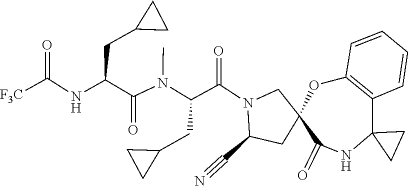 [SMILES: CN(C(=O)[C@H](CC1CC1)NC(=O)C(F)(F)F)[C@@H](CC1CC1)C(=O)N1C[C@@]2(C[C@H]1C#N)Oc1ccccc1C1(CC1)NC2=O]

Compound 113 was prepared similarly as described for Compound 81 using t-butyl (1-(2-hydroxyphenyl)cyclopropyl)carbamate in place of t-butyl (2-hydroxybenzyl)carbamate. ¹H NMR (500 MHz, 363K, DMSO-d₆) δ 8.58 (s, 1H), 7.28 (m, 1H), 7.21 (m, 1H), 7.06-7.17 (m, 2H), 5.30 (m, 1H), 5.02 (m, 1H), 4.82 (m, 1H), 4.28 (m, 1H), 4.06 (m, 1H), 3.08 (s, 3H), 2.83 (m, 1H), 2.66 (m, 1H), 1.68 (m, 2H), 1.63 (m, 2H), 1.27 (m, 2H), 1.17 (m, 1H), 1.01 (m, 1H), 0.70 (m, 1H), 0.63 (m, 1H), 0.28-0.45 (m, 3H), 0.23 (m, 1H), 0.10 (m, 2H), −0.08-0.05 (m, 2H). LCMS (ESI, m/z): 600 [M−H]⁻.

t-Butyl (1-(2-hydroxyphenyl)cyclopropyl)carbamate: To a solution of t-butyl (1-(2-(benzyloxy)phenyl)cyclopropyl)carbamate (3.0 g, 8.85 mmol) in EtOH (20 mL) were added 10% Pd/C (187 mg) and ammonium formate (2.23 g, 35.4 mmol). The mixture was heated at 70° C. for 1 h. After cooling to rt, the mixture was filtered through celite. The filtrate was concentrated under reduced pressure to afford t-butyl (1-(2-hydroxyphenyl)cyclopropyl)carbamate (2.0 g, 90%) as an off-white solid.

### Example 61

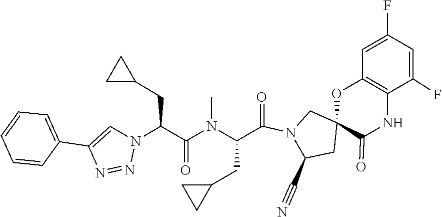 [SMILES: CN(C(=O)[C@H](CC1CC1)n1cc(-c2ccccc2)nn1)[C@@H](CC1CC1)C(=O)N1C[C@@]2(C[C@H]1C#N)Oc1cc(F)cc(F)c1NC2=O]

To a solution of (S)-2-azido-3-cyclopropylpropanoic acid (1.0 g, 6.45 mmol, 1.0 eq.) in DCM (10 mL) were added dropwise oxalyl chloride (0.82 mL, 9.67 mmol, 1.5 eq.) and one drop of DMF. The mixture was stirred at rt for 2 h and then concentrated under reduced pressure to afford quantitatively (S)-2-azido-3-cyclopropylpropanoyl chloride as a brown oil.

To a solution of (2R,5'S)-1′-((S)-3-cyclopropyl-2-(methylamino)propanoyl)-5,7-difluoro-3-oxo-3,4-dihydrospiro[benzo[b][1,4]oxazine-2,3′-pyrrolidine]-5′-carboxamide (500 mg, 1.23 mmol, 1.0 eq.) in THF (5.0 mL) cooled at −20° C. were added NEt₃ (0.34 mL, 2.45 mmol, 2.0 eq.) and a solution of (S)-2-azido-3-cyclopropylpropanoyl chloride (227 mg, 1.47 mmol, 1.2 eq.) in THF (2 mL). The mixture was stirred at −20° C. for 1 h and then at rt for 16 h. The mixture was diluted with water (15 mL) and extracted with EA (2×15 mL). The organic phases were combined, washed with brine (15 mL), dried over Na₂SO₄, filtered and concentrated under reduced pressure. The residue was purified by flash chromatography on silica gel using a gradient of MeOH (1 to 15%) in DCM to afford (2R,5'S)-1′-((S)-2-((S)-2-azido-3-cyclopropyl-N-methylpropanamido)-3-cyclopropyl propanoyl)-5,7-difluoro-3-oxo-3,4-dihydrospiro[benzo[b][1,4]oxazine-2,3′-pyrrolidine]-5′-carboxamide (500 mg, 75%) as an off-white solid.

To a solution of (2R,5'S)-1′-((S)-2-((S)-2-azido-3-cyclopropyl-N-methylpropanamido)-3-cyclopropylpropanoyl)-5,7-difluoro-3-oxo-3,4-dihydrospiro[benzo[b][1,4]oxazine-2,3′-pyrrolidine]-5′-carboxamide (100 mg, 0.183 mmol, 1.0 eq.) in t-BuOH (1 mL) and water (2 mL) were added phenyl acetylene (0.030 mL, 0.275 mmol, 1.5 eq.), sodium-L-ascorbate (3.6 mg, 0.018 mmol, 0.1 eq.) and CuSO₄·5H₂O (22 mg, 0.091 mmol, 0.5 eq). The mixture was stirred at rt for 16 h. The mixture was diluted with water (5 mL) and extracted with EA (2×10 mL). The organic phases were combined, washed with brine (10 mL), dried over Na₂SO₄, filtered and concentrated under reduced pressure. The residue was purified by flash chromatography on silica gel using a gradient of MeOH (1 to 10%) in DCM to afford (2R,5'S)-1′-((S)-3-cyclopropyl-2-((S)-3-cyclopropyl-N-methyl-2-(4-phenyl-1H-1,2,3-triazol-1-yl)propanamido)propanoyl)-5,7-difluoro-3-oxo-3,4-dihydrospiro[benzo[b][1,4]oxazine-2,3′-pyrrolidine]-5′-carboxamide (100 mg, 84%) as a white solid.

To a solution of (2R,5'S)-1′-((S)-3-cyclopropyl-2-((S)-3-cyclopropyl-N-methyl-2-(4-phenyl-1H-1,2,3-triazol-1-yl)propanamido)propanoyl)-5,7-difluoro-3-oxo-3,4-dihydrospiro[benzo[b][1,4]oxazine-2,3′-pyrrolidine]-5′-carboxamide (90 mg, 0.139 mmol, 1.0 eq.) in DCM (1 mL) cooled at 0° C. were added pyridine (0.024 mL, 0.306 mmol, 2.2 eq.) and TFAA (0.022 mL, 0.153 mmol, 1.1 eq.). The mixture was stirred at 0° C. for 1 h. The mixture was washed with water (5 mL). The phases were separated. The aqueous phase was extracted with DCM (2×5 mL). The organic phases were combined, washed with sat. NaHCO₃ (5 mL), dried over Na₂SO₄, filtered and concentrated under reduced pressure. The residue was purified by prep-HPLC (Column: Daisogel-C18, 25*150 mm, 10 m; Mobile Phase A: 10 mM NH₄HCO₃ in water, Mobile Phase B: ACN; Flow rate: 22 mL/min; Gradient: 45% B to 60% B in 10 min) to afford (S)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-N-methyl-2-(4-phenyl-1H-1,2,3-triazol-1-yl)propenamide (46 mg, 52%) as an off-white solid. ¹H NMR (500 MHz, 363K, DMSO-d₆) δ 10.80 (br. s., 1H), 8.46 (s, 1H), 7.81 (m, 2H), 7.41 (m, 2H), 7.30 (t, 1H), 6.94 (m, 1H), 6.74 (m, 1H), 5.86 (m, 1H), 5.16 (m, 1H), 4.98 (m, 1H), 4.01-4.29 (m, 2H), 3.10 (s, 3H), 2.81 (m, 1H), 2.71 (m, 1H), 2.02 (m, 1H), 1,92 (m, 1H), 1.63 (m, 2H), 0.58 (m, 1H), 0.50 (m, 1H), 0.19-0.39 (m, 4H), −0.08-0.14 (m, 4H). LCMS (ESI, m/z): 630 [M+H]⁺.

### Example 62

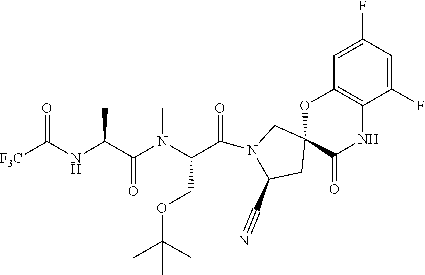 [SMILES: C[C@H](NC(=O)C(F)(F)F)C(=O)N(C)[C@@H](COC(C)(C)C)C(=O)N1C[C@@]2(C[C@H]1C#N)Oc1cc(F)cc(F)c1NC2=O]

To a solution of (2R,5'S)-5,7-difluoro-3-oxo-3,4-dihydrospiro[benzo[b][1,4]oxazine-2,3′-pyrrolidine]-5′-carboxamide hydrochloride (250 mg, 0.782 mmol, 1.0 eq.) and N-((benzyloxy)carbonyl)-O-(t-butyl)-N-methyl-L-serine (242 mg, 0.782 mmol, 1.0 eq.) in DCM (3.5 mL) and DMF (1.5 mL) cooled at 0° C. were added HATU (312 mg, 0.821 mmol, 1.05 eq.) and N-methylmorpholine (0.172 mL, 1.56 mmol, 2.0 eq.). The mixture was stirred at rt for 5 h. The mixture was diluted with water (5 mL) and extracted with EA (3×10 mL). The organic phases were combined, dried over Na₂SO₄, filtered and concentrated under reduced pressure. The residue was purified by flash chromatography on silica gel using a gradient of MeOH (2 to 3%) in DCM to afford benzyl ((S)-3-(t-butoxy)-1-((2R,5'S)-5′-carbamoyl-5,7-difluoro-3-oxo-3,4-dihydrospiro[benzo[b][1,4]oxazine-2,3′-pyrrolidin]-1′-yl)-1-oxopropan-2-yl)(methyl)carbamate (330 mg, 73%) as an off-white solid.

A mixture of benzyl ((S)-3-(t-butoxy)-1-((2R,5'S)-5′-carbamoyl-5,7-difluoro-3-oxo-3,4-dihydrospiro[benzo[b][1,4]oxazine-2,3′-pyrrolidin]-1′-yl)-1-oxopropan-2-yl)(methyl)carbamate (430 mg, 0.748 mmol, 1.0 eq.) and 10% Pd/C (59 mg) in MeOH (8 mL) was stirred at rt for 4 h under H₂ atmosphere. The mixture was filtered through celite and the solids were washed with MeOH. The filtrate was concentrated under reduced pressure. The residue was purified by flash chromatography on silica gel using a gradient of MeOH (3 to 5%) in DCM to afford (2R,5'S)-1′-(O-(t-butyl)-N-methyl-L-seryl)-5,7-difluoro-3-oxo-3,4-dihydrospiro[benzo[b][1,4]oxazine-2,3′-pyrrolidine]-5′-carboxamide (260 mg, 78%) as an off-white solid.

To a solution of (2R,5'S)-1′-(O-(t-butyl)-N-methyl-L-seryl)-5,7-difluoro-3-oxo-3,4-dihydrospiro[benzo[b][1,4]oxazine-2,3′-pyrrolidine]-5′-carboxamide (260 mg, 0.590 mmol, 1.0 eq.) and (((9H-fluoren-9-yl)methoxy)carbonyl)-L-alanine (184 mg, 0.590 mmol, 1.0 eq.) in DMF (3 mL) cooled at 0° C. were added HATU (269 mg, 0.708 mmol, 1.2 eq.) and DIPEA (0.258 mL, 1.48 mmol, 2.5 eq.). The mixture was allowed to warm to rt and then stirred at rt for 4 h. The mixture was diluted with cold water (10 mL), and extracted with EA (4×30 mL). The organic phases were combined, washed with brine, dried over Na₂SO₄, filtered and concentrated under reduced pressure. The residue was purified by flash chromatography on silica gel using a gradient of MeOH (4 to 6%) in DCM to afford (9H-fluoren-9-yl)methyl ((S)-1-(((S)-3-(t-butoxy)-1-((2R,5'S)-5′-carbamoyl-5,7-difluoro-3-oxo-3,4-dihydrospiro[benzo[b][1,4]oxazine-2,3′-pyrrolidin]-1′-yl)-1-oxopropan-2-yl)(methyl)amino)-1-oxopropan-2-yl)carbamate (390 mg, 90%) as an off-white solid.

A mixture of (9H-fluoren-9-yl)methyl ((S)-1-(((S)-3-(t-butoxy)-1-((2R,5'S)-5′-carbamoyl-5,7-difluoro-3-oxo-3,4-dihydrospiro[benzo[b][1,4]oxazine-2,3′-pyrrolidin]-1′-yl)-1-oxopropan-2-yl)(methyl)amino)-1-oxopropan-2-yl)carbamate (100 mg, 0.136 mmol, 1.0 eq.) in 20% piperidine in DMF (1 mL) was stirred at rt for 3 h. The mixture was concentrated under reduced pressure. The residue was triturated with n-pentane to afford quantitatively (2R,5'S)-1′-(N-(L-alanyl)-O-(t-butyl)-N-methyl-L-seryl)-5,7-difluoro-3-oxo-3,4-dihydrospiro[benzo[b][1,4]oxazine-2,3′-pyrrolidine]-5′-carboxamide as an off-white solid.

To a solution of (2R,5'S)-1′-(N-(L-alanyl)-O-(t-butyl)-N-methyl-L-seryl)-5,7-difluoro-3-oxo-3,4-dihydrospiro[benzo[b][1,4]oxazine-2,3′-pyrrolidine]-5′-carboxamide (140 mg, 0.274 mmol, 1.0 eq.) in methanol (3 mL) cooled at 0° C. were added ethyl 2,2,2-trifluoroacetate (0.162 mL, 1.37 mmol, 5.0 eq.), NEt₃ (0.190 mL, 1.37 mmol, 5.0 eq.) and 1-methylimidazole (0.044 mL, 0.547 mmol, 2.0 eq.). The mixture was stirred at rt for 16 h and then concentrated under reduced pressure. The residue was partitioned between with EA (50 mL) and water. The phases were separated. The organic phase was washed with water and brine, dried over Na₂SO₄, filtered and concentrated under reduced pressure. The residue was purified by flash chromatography on silica gel using a gradient of MeOH (4 to 5%) in DCM to afford (2R,5'S)-1′-(O-(t-butyl)-N-methyl-N-((2,2,2-trifluoroacetyl)-L-alanyl)-L-seryl)-5,7-difluoro-3-oxo-3,4-dihydrospiro[benzo[b][1,4]oxazine-2,3′-pyrrolidine]-5′-carboxamide (85 g, 43%) as an off-white solid.

To a solution of (2R,5'S)-1′-(O-(t-butyl)-N-methyl-N-((2,2,2-trifluoroacetyl)-L-alanyl)-L-seryl)-5,7-difluoro-3-oxo-3,4-dihydrospiro[benzo[b][1,4]oxazine-2,3′-pyrrolidine]-5′-carboxamide (80 mg, 0.132 mmol, 1.0 eq.) in DCM (3.2 mL) cooled at 0° C. were added pyridine (0.023 mL, 0.290 mmol, 2.2 eq) and TFAA (0.020 mL, 0.145 mmol, 1.1 eq). The mixture was stirred at 0° C. for 1 h. The mixture was washed with saturated NaHCO₃ (5 mL). The phases were separated. The aqueous phase was extracted with DCM (3×10 mL). The organic phases were combined, washed with brine, dried over Na₂SO₄, filtered and concentrated under reduced pressure. The residue was purified by prep-HPLC (Column: X-SELECT CSH C18, 19*150 mm, 5 m; Mobile Phase A: 10 mM NH₄HCO₃ in water, Mobile Phase B: ACN; Flow rate: 15 mL/min; Gradient: 15% B to 60% B in 10 min) to afford (S)—N—((S)-3-(t-butoxy)-1-((2R,5'S)-5′-cyano-5,7-difluoro-3-oxo-3,4-dihydrospiro[benzo[b][1,4]oxazine-2,3′-pyrrolidin]-1′-yl)-1-oxopropan-2-yl)-N-methyl-2-(2,2,2-trifluoroacetamido)propenamide (50 mg, 64%) as a white solid. ¹H NMR (500 MHz, 363K, DMSO-d₆) δ 10.40 (br. s., 1H), 9.20 (br. s., 1H), 6.92 (t, 1H), 6.72 (m, 1H), 4.89-5.13 (m, 2H), 4.77 (m, 1H), 3.95-4.13 (m, 2H), 3.71 (m, 1H), 3.60 (m, 1H), 3.02 (s, 3H), 2.67-2.85 (m, 2H), 1.23 (m, 3H), 1.14 (s, 9H). LCMS (ESI, m/z): 588 [M−H]⁻.

### Example 63

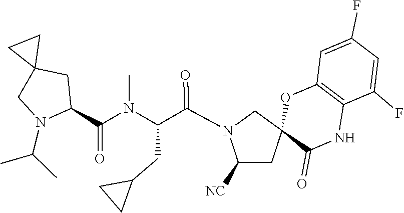 [SMILES: CC(C)N1CC2(CC2)C[C@H]1C(=O)N(C)[C@@H](CC1CC1)C(=O)N1C[C@@]2(C[C@H]1C#N)Oc1cc(F)cc(F)c1NC2=O]

Compound 116 was prepared similarly as described for Compound 101 using (S)-5-isopropyl-5-azaspiro[2.4]heptane-6-carboxylic acid in place of (S)-3-cyclopropyl-2-(2-oxooxazolidin-3-yl)propanoic acid. ¹H NMR (500 MHz, 363K, DMSO-d₆) δ 11.00 (br. s., 1H), 6.93 (m, 1H), 6.69 (m, 1H), 5.21 (m, 1H), 4.93 (m, 1H), 4.11 (m, 2H), 3.93 (m, 1H), 3.01 (s, 3H), 2.79-2.90 (m, 3H), 2.57-2.74 (m, 2H), 2.05 (m, 1H), 1.56-1.73 (m, 2H), 1.50 (m, 1H), 0.84-1.03 (m, 6H), 0.62 (m, 1H), 0.47 (m, 2H), 0.32-0.44 (m, 3H), 0.28 (m, 1H), 0.10 (m, 2H). LCMS (ESI, m/z): 556 [M+H]⁺.

(S)-5-Isopropyl-5-azaspiro[2.4]heptane-6-carboxylic acid: To a solution of benzyl (S)-5-azaspiro[2.4]heptane-6-carboxylate hydrochloride (500 mg, 2.16 mmol, 1.0 eq.) in MeOH (5 mL) cooled at 0° C. was added acetone (0.12 mL, 2.16 mmol, 1.0 eq.). The mixture was stirred at 0° C. for 20 min. NaBH₃CN (149 mg, 2.37 mmol, 1.1 eq.) was added and the mixture was stirred at rt for 16 h. The mixture was diluted with water (10 mL) and extracted with EA (2×20 mL). The organic phases were combined, dried over Na₂SO₄, filtered and concentrated under reduced pressure. The residue was purified by flash chromatography on silica gel using a gradient of EA (10 to 15%) in PE to afford benzyl (S)-5-isopropyl-5-azaspiro[2.4]heptane-6-carboxylate (160 mg, 27%) as a yellow oil.

A mixture of benzyl (S)-5-isopropyl-5-azaspiro[2.4]heptane-6-carboxylate (140 mg, 0.512 mmol) and 10% Pd/C (28 mg) in MeOH (1.4 mL) was stirred at rt for 2 h under H₂ atmosphere. The mixture was filtered and the filtrate was concentrated under reduced pressure. The residue was taken up with EA (3 mL). The solid was filtered and dried under high vacuum to afford (S)-5-isopropyl-5-azaspiro[2.4]heptane-6-carboxylic acid (65 mg, 70%) as an off-white solid.

### Example 64

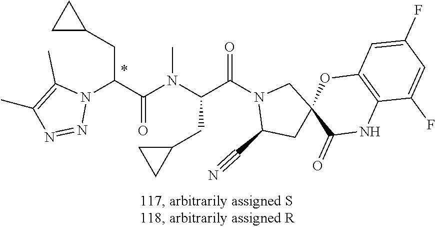 [SMILES: Cc1nnn(C(CC2CC2)C(=O)N(C)[C@@H](CC2CC2)C(=O)N2C[C@@]3(C[C@H]2C#N)Oc2cc(F)cc(F)c2NC3=O)c1C]

A solution of 4-methylbenzenesulfonohydrazide (650 mg, 3.50 mmol, 1.0 eq.) and 3,3-dimethoxybutan-2-one (1.03 mL, 7.69 mmol, 2.2 eq.) in MeOH was stirred at rt for 15 min. Methyl (S)-2-amino-3-cyclopropylpropanoate (500 mg, 3.50 mmol, 1.0 eq.) and NEt₃ (0.53 mL, 3.85 mmol, 1.1 eq.) were added. The mixture was heated at 14 0° C. for 30 min under microwave irradiation. After cooling to rt, the mixture was diluted with water (50 mL) and extracted with EA (2×30 mL). The organic phases were combined, washed with brine (2×20 mL), dried over Na₂SO₄, filtered and concentrated under reduced pressure. The residue was purified by flash chromatography on silica gel using a gradient of EA (30 to 50%) in PE to afford methyl 3-cyclopropyl-2-(4,5-dimethyl-1H-1,2,3-triazol-1-yl)propanoate (500 mg, 64%) as a colorless oil.

To a solution of methyl 3-cyclopropyl-2-(4,5-dimethyl-1H-1,2,3-triazol-1-yl)propanoate (200 mg, 0.892 mmol) in THF (1 mL) and water (1 mL) cooled at 0° C. was added LiOH (56 mg, 1.34 mmol). The mixture was stirred at rt for 1 h. The mixture was partially concentrated under reduced pressure. The residue was acidified with 1N HCl until pH 5 and extracted with EA (15 mL). The phases were separated. The aqueous phase was extracted with EA (15 mL). The organic phases were combined, dried over Na₂SO₄, filtered and concentrated under reduced pressure to afford 3-cyclopropyl-2-(4,5-dimethyl-1H-1,2,3-triazol-1-yl)propanoic acid (170 mg, 90%) as a yellow solid.

To a solution of 3-cyclopropyl-2-(4,5-dimethyl-1H-1,2,3-triazol-1-yl)propanoic acid (92 mg, 0.441 mmol, 1.2 eq.) and (2R,5'S)-1′-((S)-3-cyclopropyl-2-(methylamino)propanoyl)-5,7-difluoro-3-oxo-3,4-dihydrospiro[benzo[b][1,4]oxazine-2,3′-pyrrolidine]-5′-carboxamide hydrochloride (150 mg, 0.367 mmol, 1.0 eq.) in DMF (1.5 mL) cooled at 0° C. were added HATU (167 mg, 0.441 mmol, 1.2 eq.) and DIPEA (0.160 mL, 0.917 mmol, 2.5 eq.). The mixture was stirred at rt for 3 h. The mixture was diluted with water (10 mL) and extracted with EA (3×15 mL). The organic phases were combined, washed with brine (15 mL), dried over Na₂SO₄, filtered and concentrated under reduced pressure. The residue was purified by flash chromatography on silica gel using a gradient of MeOH (1 to 10%) in DCM to afford (2R,5'S)-1′-((2S)-3-cyclopropyl-2-(3-cyclopropyl-2-(4,5-dimethyl-1H-1,2,3-triazol-1-yl)-N-methylpropanamido)propanoyl)-5,7-difluoro-3-oxo-3,4-dihydrospiro[benzo[b][1,4]oxazine-2,3′-pyrrolidine]-5′-carboxamide (150 mg, 66%) as a white solid.

To a solution of (2R,5'S)-1′-((2S)-3-cyclopropyl-2-(3-cyclopropyl-2-(4,5-dimethyl-1H-1,2,3-triazol-1-yl)-N-methylpropanamido)propanoyl)-5,7-difluoro-3-oxo-3,4-dihydrospiro[benzo[b][1,4]oxazine-2,3′-pyrrolidine]-5′-carboxamide (150 mg, 0.250 mmol, 1.0 eq.) in DCM (1.5 mL) cooled at 0° C. were added pyridine (0.044 mL, 0.550 mmol) and TFAA (0.038 mL, 0.275 mmol). The mixture was allowed to warm to rt over 1 h. The mixture was washed with water (10 mL). The phases were separated. The aqueous phase was extracted with DCM (2×20 mL). The organic phases were combined, washed with brine (2×5 mL), dried over Na₂SO₄, filtered and concentrated under reduced pressure. The residue was purified by flash chromatography on silica gel using a gradient of MeOH (1 to 10%) in DCM and by prep-SFC using the following conditions: Column: Chiralpak AD-H, 2.5×15 cm, 5 μm; Mobile Phase A: CO₂, Mobile Phase B: iPrOH; Flow rate: 70 g/min; Elution condition: isocratic 20% B; Column Temperature: 30° C.; Purification resulted in (S*)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-2-(4,5-dimethyl-1H-1,2,3-triazol-1-yl)-N-methylpropanamide (30 mg, 22%, Compound 117) and ((R*)—N—((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)-3-cyclopropyl-2-(4,5-dimethyl-1H-1,2,3-triazol-1-yl)-N-methylpropanamide (80 mg, 50%, Compound 118).

Compound 117: ¹H NMR (500 MHz, 363K, DMSO-d₆) δ 11.00 (br. s., 1H), 6.96 (m, 1H), 6.74 (d, 1H), 5.51 (t, 1H), 5.12 (m, 1H), 4.97 (m, 1H), 4.27 (m, 1H), 4.05 (m, 1H), 2.86 (s, 3H), 2.72-2.83 (m, 2H), 2.19 (s, 3H), 2.15 (s, 3H), 1.85-2.05 (m, 2H), 1.61 (m, 2H), 0.53 (m, 2H), 0.24-0.40 (m, 4H), 0.00-0.15 (m, 3H), −0.12 (m, 1H). LCMS (ESI, m/z): 582 [M+H]⁺. SFC: CHIRALPAK AD-H, 4.6×150 mm, 5 m, 30° C., co-Solvent: 0.5% iPrNH₂ in iPrOH, hold 8 min at 20%, Rt: 1.94 min.

Compound 118: ¹H NMR (500 MHz, 363K, DMSO-d₆) δ 11.00 (br. s., 1H), 6.92 (m, 1H), 6.61 (d, 1H), 5.51 (t, 1H), 5.12 (m, 1H), 4.89 (m, 1H), 4.05-4.14 (m, 2H), 2.87 (s, 3H), 2.55-2.77 (m, 2H), 2.15 (s, 3H), 1.99 (s, 3H), 1.96 (m, 2H), 1.55-1.74 (m, 2H), 0.66 (m, 1H), 0.55 (m, 1H), 0.24-0.46 (m, 4H), 0.05-0.16 (m, 3H), −0.10 (m, 1H). LCMS (ESI, m/z): 582 [M+H]⁺. SFC: CHIRALPAK AD-H, 4.6×150 mm, 5 m, 30° C., co-Solvent: 0.5% iPrNH₂ in iPrOH, hold 8 min at 20%, Rt: 3.48 min.

### Example 65

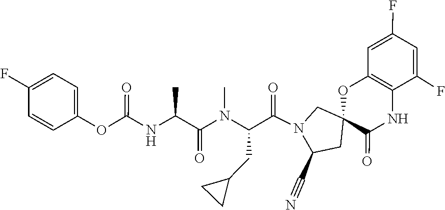 [SMILES: C[C@H](NC(=O)Oc1ccc(F)cc1)C(=O)N(C)[C@@H](CC1CC1)C(=O)N1C[C@@]2(C[C@H]1C#N)Oc1cc(F)cc(F)c1NC2=O]

To a mixture of (2R,5'S)-1′-((S)-2-((S)-2-amino-N-methylpropanamido)-3-cyclopropylpropanoyl)-5,7-difluoro-3-oxo-3,4-dihydrospiro[benzo[b][1,4]oxazine-2,3′-pyrrolidine]-5′-carboxamide (83.0 mg, 0.173 mmol) and triethylamine (52.5 mg, 0.519 mmol) in DCM (5 mL) was added 4-fluorophenyl carbonochloridate (30.2 mg, 0.173 mmol) in DCM (0.5 mL) at 0° C. The mixture was stirred for 1 h at rt and the reaction was quenched with water (6 mL). The mixture was extracted with DCM (3×6 mL). The organic layers were combined, dried over anhydrous sodium sulfate, filtered and concentrated under reduced pressure to afford the crude product. The crude product was chromatographed on a silica gel column with MeOH:DCM (1:12) to provide 4-fluorophenyl ((S)-1-(((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-1-oxopropan-2-yl)carbamate (90.0 mg, 84%) as a white solid. LC-MS (ESI, m/z): 640[M+Na]⁺.

To a mixture of 4-fluorophenyl ((S)-1-(((S)-1-((2R,5'S)-5′-carbamoyl-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-1-oxopropan-2-yl)carbamate (90.0 mg, 0.146 mmol, 1.0 eq.) in DCM (8 mL) was added pyridine (58.0 mg, 0.730 mmol, 5.0 eq.) and trifluoroacetic anhydride (61.0 mg, 0.292 mmol, 2.0 eq.). The mixture was stirred for 1 h at rt and the reaction was quenched with water (10 mL). The mixture was extracted with DCM (3×10 mL). The organic layers were combined, dried over anhydrous sodium sulfate, filtered and concentrated under reduced pressure to afford the crude product. The crude product was purified by preparative HPLC (Column: XselectCSH Prep OBD C18 Column, 30×150 mm, 5 m; Mobile Phase A: Water (0.1% FA), Mobile Phase B: ACN; Flow rate: 60 mL/min; Gradient: 44% B to 50% B in 10 min; Wave Length: 220 nm; RT1(min): 9.3) to provide 4-fluorophenyl ((S)-1-(((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-1-oxopropan-2-yl)carbamate (19.9 mg, 22%) as a white solid. ¹H NMR (400 MHz, 80° C., DMSO-d₆) δ 10.50-11.50 (m, 1H), 7.50-7.80 (m, 1H), 6.95-7.15 (m, 2H), 6.50-6.94 (m, 3H), 5.00-5.25 (m, 1H), 4.75-4.99 (m, 1H), 4.30-4.55 (m, 1H), 3.75-4.28 (m, 2H), 2.80-2.95 (m, 3H), 2.45-2.79 (m, 2H), 1.40-1.70 (m, 2H), 0.75-1.25 (m, 3H), 0.45-0.70 (m, 1H), 0.15-0.44 (m, 2H), −0.05-0.14 (m, 2H). LC-MS (ESI, m/z): 622 [M+Na]⁺.

### Example 66

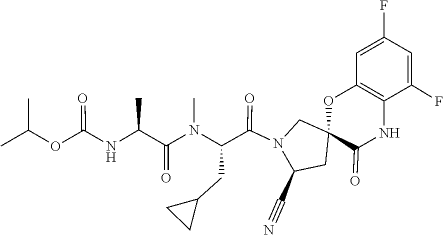 [SMILES: CC(C)OC(=O)N[C@@H](C)C(=O)N(C)[C@@H](CC1CC1)C(=O)N1C[C@@]2(C[C@H]1C#N)Oc1cc(F)cc(F)c1NC2=O]

Compound 120 was prepared similarly as described for Compound 119 using isopropyl chloroformate instead of phenyl chloroformate. The crude product was purified by prep-HPLC (Column: XselectCSH Prep OBD C18 Column, 30×150 mm, 5 m; Mobile Phase A: Water(0.1% FA), Mobile Phase B: ACN; Flow rate: 60 mL/min; Gradient: 42% B to 50% B in 8 min; Wave Length: 220 nm nm; RT1(min): 7.2) to provide isopropyl ((S)-1-(((S)-1-((2R,5'S)-5′-cyano-5,7-difluoro-3-oxo-3,4-dihydrospiro[benzo[b][1,4]oxazine-2,3′-pyrrolidin]-1′-yl)-3-cyclopropyl-1-oxopropan-2-yl)(methyl)amino)-1-oxopropan-2-yl)carbamate (6.1 mg, 13%) as a white solid. ¹H NMR (400 MHz, 80° C., DMSO-d₆) δ 11.00 (br, 1H), 6.80-7.02 (m, 1H), 6.40-6.78 (m, 2H), 4.75-5.30 (m, 2H), 4.55-4.70 (m, 1H), 4.25-4.40 (m, 1H), 3.80-4.10 (m, 2H), 2.80-2.95 (m, 3H), 2.50-2.79 (m, 2H), 1.45-1.70 (m, 2H), 1.05-1.15 (m, 6H), 0.80-1.04 (m, 3H), 0.48-0.65 (m, 1H), 0.16-0.40 (m, 2H), −0.08-0.08 (m, 2H). LC-MS (ESI, m/z): 570 [M+Na]⁺.

### Example 67

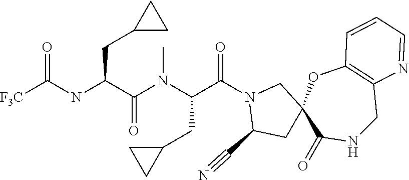

To a solution of 3-hydroxypicolinonitrile (10.0 g, 84.0 mmol) in MeOH (120 mL) and ammonium hydroxide (22 mL) was added Raney nickel (15.0 g) under hydrogen. The mixture was stirred 18 h at rt. The mixture was filtered through a celite pad and washed with MeOH (3×50 mL). The filtrate was added di-t-butyl dicarbonate (27.5 g, 126 mmol, 1.5 eq.) and triethylaminethe (36.6 g, 168 mmol). The mixture was stirred for 1 h at rt and the reaction was quenched with water (300 mL). The mixture was extracted with EA (3×500 mL). The organic layers were combined, washed with brine (2×500 mL), dried over anhydrous sodium sulfate, filtered and concentrated under reduced pressure to afford the crude product, which was chromatographed on a silica gel column with EA:PE (40-50%) to provide t-butyl ((3-hydroxypyridin-2-yl)methyl)carbamate (15 g, 79%) as a white solid. ¹H NMR (400 MHz, DMSO-d₆) δ 9.96 (s, 1H), 7.88-8.02 (m, 1H), 7.11-7.14 (m, 1H), 6.76-6.85 (m, 1H), 6.13 (br, 1H), 4.12-4.25 (m, 2H), 1.38-1.42 (m, 9H). LC-MS (ESI, m/z): 225 [M+H]⁺.

To a solution of t-butyl ((3-hydroxypyridin-2-yl)methyl)carbamate (12.0 g, 53.8 mmol) in THF (120 mL) was added sodium hydride (1.49 g, 64.6 mmol, 1.2 eq.) at 0° C. The mixture was stirred for 1 h at rt and then concentrated under reduced pressure to afford sodium 2-(((t-butoxycarbonyl)amino)methyl)pyridin-3-olaten (crude). To a mixture of sodium 2-(((t-butoxycarbonyl)amino)methyl)pyridin-3-olaten (crude) and 1-(t-butyl) 2-methyl (2S,4S)-4-hydroxy-4-(trichloromethyl)pyrrolidine-1,2-dicarboxylate (15.0 g, 41.4 mmol) in acetone (150 mL) was added NaOH (6.62 g, 166 mmol) at 0° C. The mixture was stirred for 1 h at rt and acidified to pH=5 with HCl (4 M in 1,4-dioxane) at 0° C. The mixture was concentrated under reduced pressure to afford (2S,4R)-1-(t-butoxycarbonyl)-4-((2-(((t-butoxycarbonyl)amino)methyl)pyridin-3-yl)oxy)pyrrolidine-2,4-dicarboxylic acid (19.5 g, crude) as a yellow oil. LC-MS (ESI, m/z): 482 [M+H]⁺.

To a solution of (2S,4R)-1-(t-butoxycarbonyl)-4-((2-(((t-butoxycarbonyl)amino)methyl)pyridin-3-yl)oxy)pyrrolidine-2,4-dicarboxylic acid (19.5 g, 40.5 mmol) and potassium carbonate (45.0 g, 324 mmol) in DMF (300 mL) was added iodomethane (28.7 g, 203 mmol) at 0° C. The mixture was stirred for 3 h at rt and the reaction was quenched with water (500 mL). The mixture was extracted with EA (3×1 L). The organic layers were combined, washed with brine (2×1 L), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide 1-(t-butyl) 2,4-dimethyl (2S,4R)-4-((2-(((t-butoxycarbonyl)amino)methyl)pyridin-3-yl)oxy)pyrrolidine-1,2,4-tricarboxylate (2.90 g, 14%) as a yellow solid. ¹H NMR (400 MHz, DMSO-d₆) δ 8.05-8.26 (m, 1H), 7.18-7.33 (m, 1H), 7.05-7.12 (m, 1H), 6.83-6.98 (m, 1H), 3.94-4.54 (m, 5H), 3.62-3.86 (m, 6H), 2.78-2.89 (m, 1H), 2.51-2.62 (m, 1H), 1.32-1.42 (m, 9H). LC-MS (ESI, m/z): 510 [M+H]⁺.

To a solution of 1-(t-butyl) 2,4-dimethyl (2S,4R)-4-((2-(((t-butoxycarbonyl)amino)methyl)pyridin-3-yl)oxy)pyrrolidine-1,2,4-tricarboxylate (2.90 g, 5.70 mmol) in 1,4-dioxane (10 mL) and was added HCl (20 mL 4 M in 1,4-dioxane). The mixture was stirred for 1 h at rt and then concentrated under reduced pressure to afford dimethyl (2S,4R)-4-((2-(aminomethyl)pyridin-3-yl)oxy)pyrrolidine-2,4-dicarboxylate (1.75 g, crude) as a yellow solid. LC-MS (ESI, m/z): 310 [M+H]⁺.

A mixture of dimethyl (2S,4R)-4-((2-(aminomethyl)pyridin-3-yl)oxy)pyrrolidine-2,4-dicarboxylate (1.75 g, 5.7 mmol) in MeOH (30 mL) was added DIEA (6.58 g, 51.0 mmol). The mixture was stirred for overnight at rt. Ammonia (100 mL, 7M in MeOH) was then added. The mixture was stirred overnight at 50° C. and then concentrated under reduced pressure to afford (2R,5'S)-3-oxo-4,5-dihydro-3H-spiro[pyrido[2,3-f][1,4]oxazepine-2,3′-pyrrolidine]-5′-carboxamide (1.45 g, crude) as a yellow oil. LC-MS (ESI, m/z): 263 [M+H]⁺.

To a solution of (2R,5'S)-3-oxo-4,5-dihydro-3H-spiro[pyrido[2,3-f][1,4]oxazepine-2,3′-pyrrolidine]-5′-carboxamide (1.45 g, 5.53 mmol) in DCM (20 mL) were added triethylamine (1.12 g, 11.1 mmol) and di-t-butyl dicarbonate (1.81 g, 8.30 mmol). The mixture was stirred for 1 h at rt and then concentrated under reduced pressure to afford the crude product. The crude product was purified by TLC (Mobile phase: MeOH:DCM=1:10; Rf=0.3; detection: UV) to provide t-butyl (2R,5'S)-5′-carbamoyl-3-oxo-4,5-dihydro-3H-spiro[pyrido[2,3-f][1,4]oxazepine-2,3′-pyrrolidine]-1′-carboxylate (1.2 g, 59%) as a yellow solid. ¹H NMR (400 MHz, DMSO-d₆) δ 9.80-10.21 (m, 1H), 8.30-8.45 (m, 1H), 7.40-7.65 (m, 3H), 6.95-7.02 (m, 1H), 4.25-4.50 (m, 3H), 3.75-3.95 (m, 1H), 3.02-3.60 (m, 1H), 2.53-2.55 (m, 1H), 2.40-2.48 (m, 1H), 1.25-1.32 (m, 9H). LC-MS (ESI, m/z): 363 [M+H]⁺.

To a solution of t-butyl (2R,5'S)-5′-carbamoyl-3-oxo-4,5-dihydro-3H-spiro[pyrido[2,3-f][1,4]oxazepine-2,3′-pyrrolidine]-1′-carboxylate (200 mg, 0.552 mmol, 1.0 eq.) in DCM (3 mL) was added trifluoroacetic acid (TFA, 1 mL). The mixture was stirred for 1 h at rt and then concentrated under reduced pressure to afford (2R,5'S)-3-oxo-4,5-dihydro-3H-spiro[pyrido[2,3-f][1,4]oxazepine-2,3′-pyrrolidine]-5′-carboxamide (145 mg, crude) as a yellow solid. LC-MS (ESI, m/z): 263 [M+H]⁺.

To a solution of (2R,5'S)-3-oxo-4,5-dihydro-3H-spiro[pyrido[2,3-f][1,4]oxazepine-2,3′-pyrrolidine]-5′-carboxamide (140 mg, 0.540 mmol, 1.0 eq.), (S)-2-((t-butoxycarbonyl)(methyl)amino)-3-cyclopropylpropanoic acid (123 mg, 0.540 mmol, 1.0 eq.) and HATU (244 mg, 0.640 mmol) in DMF (3 mL) was added DIEA (345 mg, 2.67 mmol) at 0° C. The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was chromatographed on a silica gel column with MeOH:DCM (1:6) to provide t-butyl ((S)-1-((2R,5'S)-5′-carbamoyl-3-oxo-4,5-dihydro-3H-spiro[pyrido[2,3-f][1,4]oxazepine-2,3′-pyrrolidin]-1′-yl)-3-cyclopropyl-1-oxopropan-2-yl)(methyl)carbamate (117 mg, 68%) as a white solid. ¹H NMR (400 MHz, DMSO-d₆) δ 8.67-8.70 (m, 1H), 8.30-8.34 (m, 1H), 7.21-7.55 (m, 3H), 6.99 (s, 1H), 4.67-5.38 (m, 1H), 4.39-4.50 (m, 2H), 4.20-4.31 (m, 1H), 3.93-4.09 (m, 2H), 2.66-2.76 (m, 3H), 2.54-2.61 (m, 1H), 2.25-2.41 (m, 1H), 1.26-1.65 (m, 10H), 0.50-0.64 (m, 1H), 0.29-0.45 (m, 2H), 0.03-0.18 (m, 2H). LC-MS (ESI, m/z): 488 [M+H]⁺.

To a solution of t-butyl ((S)-1-((2R,5'S)-5′-carbamoyl-3-oxo-4,5-dihydro-3H-spiro[pyrido[2,3-f][1,4]oxazepine-2,3′-pyrrolidin]-1′-yl)-3-cyclopropyl-1-oxopropan-2-yl)(methyl)carbamate (140 mg, 0.290 mmol) in DCM (3 mL) was added trifluoroacetic acid (1 mL). The mixture was stirred for 1 h at rt and then concentrated under reduced pressure to afford (2R,5'S)-1′-((S)-3-cyclopropyl-2-(methylamino)propanoyl)-3-oxo-4,5-dihydro-3H-spiro[pyrido[2,3-f][1,4]oxazepine-2,3′-pyrrolidine]-5′-carboxamide (105 mg, crude) as a yellow solid. LC-MS (ESI, m/z): 388 [M+H]⁺.

To a solution of (2R,5'S)-1′-((S)-3-cyclopropyl-2-(methylamino)propanoyl)-3-oxo-4,5-dihydro-3H-spiro[pyrido[2,3-f][1,4]oxazepine-2,3′-pyrrolidine]-5′-carboxamide (105 mg, 0.270 mmol), (S)-2-((t-butoxycarbonyl)amino)-3-cyclopropylpropanoic acid (62.1 mg, 0.270 mmol), and HATU (124 mg, 0.330 mmol) in DMF (3 mL) was added DIEA (175 mg, 1.36 mmol) at 0° C. The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was chromatographed on a silica gel column with MeOH:DCM (1:7) to provide t-butyl ((S)-1-(((S)-1-((2R,5'S)-5′-carbamoyl-3-oxo-4,5-dihydro-3H-spiro[pyrido[2,3-f][1,4]oxazepine-2,3′-pyrrolidin]-1′-yl)-3-cyclopropyl-1-oxopropan-2-yl)(methyl)amino)-3-cyclopropyl-1-oxopropan-2-yl)carbamate (90 mg, 57%) as a yellow solid. ¹H NMR (400 MHz, DMSO-d₆) δ 8.56-8.72 (m, 1H), 8.20-8.38 (m, 1H), 7.40-7.73 (m, 3H), 6.96-7.10 (m, 2H), 5.10-5.38 (m, 1H), 4.34-4.44 (m, 2H), 4.11-4.25 (m, 3H), 3.90-3.99 (m, 1H), 2.90-3.02 (m, 3H), 2.13-2.41 (m, 2H), 1.32-1.70 (m, 13H), 0.60-0.77 (m, 2H), 0.07-0.46 (m, 6H), −0.36-−0.08 (m, 2H). LC-MS (ESI, m/z): 621 [M+Na]⁺.

To a solution of t-butyl ((S)-1-(((S)-1-((2R,5'S)-5′-carbamoyl-3-oxo-4,5-dihydro-3H-spiro[pyrido[2,3-f][1,4]oxazepine-2,3′-pyrrolidin]-1′-yl)-3-cyclopropyl-1-oxopropan-2-yl)(methyl)amino)-3-cyclopropyl-1-oxopropan-2-yl)carbamate (80.0 mg, 0.134 mmol, 1.0 eq.) in DCM (3 mL) was added trifluoroacetic acid (1 mL). The mixture was stirred for 1 h at rt and then concentrated under reduced pressure to afford (2R,5'S)-1′-((S)-2-((S)-2-amino-3-cyclopropyl-N-methylpropanamido)-3-cyclopropylpropanoyl)-3-oxo-4,5-dihydro-3H-spiro[pyrido[2,3-f][1,4]oxazepine-2,3′-pyrrolidine]-5′-carboxamide (65 mg crude) as a yellow solid. LC-MS (ESI, m/z): 499 [M+H]⁺.

To a solution of (2R,5'S)-1′-((S)-2-((S)-2-amino-3-cyclopropyl-N-methylpropanamido)-3-cyclopropylpropanoyl)-3-oxo-4,5-dihydro-3H-spiro[pyrido[2,3-f][1,4]oxazepine-2,3′-pyrrolidine]-5′-carboxamide (65.0 mg, 0.130 mmol) in MeOH were added triethylamine (158 mg, 1.56 mmol) and ethyl 2,2,2-trifluoroacetate (185 mg, 1.30 mmol). The mixture was stirred for overnight at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was chromatographed on a silica gel column with MeOH:DCM (1:19) to provide (2R,5'S)-1′-((S)-3-cyclopropyl-2-((S)-3-cyclopropyl-N-methyl-2-(2,2,2-trifluoroacetamido)propanamido)propanoyl)-3-oxo-4,5-dihydro-3H-spiro[pyrido[2,3-f][1,4]oxazepine-2,3′-pyrrolidine]-5′-carboxamide (75 mg, crude) as a yellow solid. LC-MS (ESI, m/z): 617 [M+Na]⁺.

To a solution of (2R,5'S)-1′-((S)-3-cyclopropyl-2-((S)-3-cyclopropyl-N-methyl-2-(2,2,2-trifluoroacetamido)propanamido)propanoyl)-3-oxo-4,5-dihydro-3H-spiro[pyrido[2,3-f][1,4]oxazepine-2,3′-pyrrolidine]-5′-carboxamide (60.0 mg, 0.101 mmol, 1.0 eq.) in DCM (3 mL) were added pyridine (40.0 mg, 0.510 mmol, 5.0 eq.) and trifluoroacetic anhydride (42.4 mg, 0.200 mmol, 2.0 eq.). The mixture was stirred for 1 h at rt and the reaction was quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was purified by preparative HPLC (Column: Xselect CSH Prep C18 Column, 30*150 mm, 5 m; Mobile Phase A: Water(0.1% FA), Mobile Phase B: ACN; Flow rate: 60 mL/min mL/min; Gradient: 30% B to 55% B in 10 min; Wave Length: 254 nm/220 nm nm; RT1(min): 8.68) to provide (S)—N—((S)-1-((2R,5'S)-5′-cyano-3-oxo-4,5-dihydro-3H-spiro[pyrido[2,3-f][1,4]oxazepine-2,3′-pyrrolidin]-1′-yl)-3-cyclopropyl-1-oxopropan-2-yl)-3-cyclopropyl-N-methyl-2-(2,2,2-trifluoroacetamido)propanamide (25.7 mg, 44%) as a white solid. ¹H NMR (400 MHz, 80° C. DMSO-d₆) δ 9.35 (br, 1H), 8.42-8.78 (m, 1H), 8.15-8.40 (m, 1H), 7.51-7.80 (m, 1H), 7.30-7.50 (m, 1H), 5.28 (br, 1H), 4.98-5.19 (m, 1H), 4.82 (br, 1H), 4.39-4.65 (m, 1H), 4.20-4.30 (m, 2H), 3.90-4.18 (m, 1H), 3.01-3.05 (m, 3H), 2.79-2.90 (m, 1H), 2.61-2.75 (m, 1H), 1.40-1.89 (m, 4H), 0.52-0.81 (m, 2H), 0.15-0.45 (m, 4H), 0.05-0.13 (m, 2H), −0.25-−0.01 (m, 2H). LC-MS (ESI, m/z): 599 [M+Na]⁺.

### Example 68

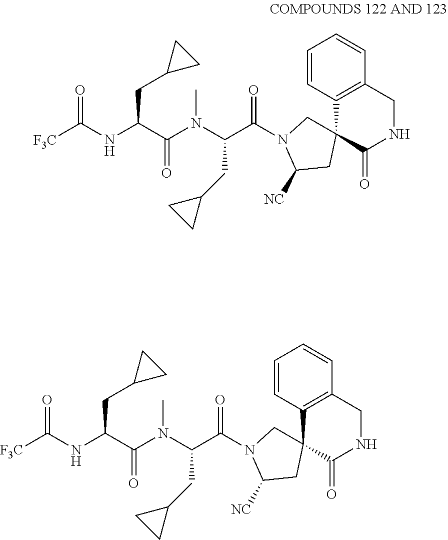 [SMILES: CN(C(=O)[C@H](CC1CC1)NC(=O)C(F)(F)F)[C@@H](CC1CC1)C(=O)N1C[C@]2(C[C@H]1C#N)C(=O)NCc1ccccc12 ; CN(C(=O)[C@H](CC1CC1)NC(=O)C(F)(F)F)[C@@H](CC1CC1)C(=O)N1C[C@@]2(C[C@@H]1C#N)C(=O)NCc1ccccc12]

To a mixture of methyl 2-(2-bromophenyl)acetate (50.0 g, 219 mmol) in dichloromethane (500 mL) were added calcium oxide (12.3 g, 219 mmol), potassium carbonate (30.3 g, 219 mmol) and paraformaldehyde (19.3 g, 438 mmol). The mixture was stirred for 4 h at 40° C. and the reaction was quenched with water (500 mL). The mixture was extracted with EA (3×800 mL). The organic layers were combined, washed with brine (2×500 mL), dried over magnesium sulfate anhydrous, filtered and concentrated under reduced pressure to afford the crude product which was chromatographed on a silica gel column with EA:PE (13:87) to provide methyl 2-(2-bromophenyl)acrylate (30.0 g, 56%) as a colorless oil. ¹H NMR (400 MHz, DMSO-d₆) δ 7.62-7.78 (m, 1H), 7.25-7.60 (m, 3H), 6.42-6.60 (m, 1H), 5.80-5.98 (m, 1H), 3.58-3.85 (m, 3H). LC-MS (ESI, m/z): 241 [M+H]⁺.

To a solution of methyl 2-(2-bromophenyl)acrylate (20.0 g, 83.3 mmol) and methyl (t-butoxycarbonyl)-L-serinate (27.4 g, 125 mmol) in DMF(600 mL) was added potassium t-butoxide (14.0 g, 125 mmol) at 0° C. The mixture was stirred for 1 h at rt and the reaction was quenched with ammonium chloride aqueous solution (500 mL). The mixture was extracted with EA (3×800 mL). The organic layers were combined, washed with brine (2×500 mL), dried over anhydrous sodium sulfate, filtered and concentrated under reduced pressure to afford the crude product. The crude product was purified by C18 column with CH₃CN/Water (0.05% TFA), (62%). The fraction was concentrated under reduced pressure to provide the crude product. The crude product was purified by prep-HPLC (Column: Xselect CSH Prep OBD C18 Column 30*150 mm, 5 m; Mobile Phase A: Water (0.1% FA), Mobile Phase B: ACN; Flow rate: 60 mL/min; Gradient: 52% B to 60% B in 8 min; Wave Length: 220 nm; RT1(min): 7.63) to provide 1-(t-butyl) 2,4-dimethyl 4-(2-bromophenyl)pyrrolidine-1,2,4-tricarboxylate (1.1 g, 3%) as a light yellow oil. ¹H NMR (400 MHz, DMSO-d₆) δ 7.59-7.80 (m, 1H), 7.20-7.55 (m, 3H), 3.85-4.42 (m, 3H), 3.55-3.74 (m, 6H), 2.95-3.08 (m, 1H), 2.52-2.70 (m, 1H), 1.20-1.47 (m, 9H). LC-MS (ESI, m/z): 342 [M-100+H]⁺.

To a mixture of 1-(t-butyl) 2,4-dimethyl 4-(2-bromophenyl)pyrrolidine-1,2,4-tricarboxylate (990 mg, 2.24 mmol) in N,N-Dimethylacetamide (50 mL) were added zinc (17.6 mg, 0.269 mmol), zinc cyanide (316 mg, 2.69 mmol), tris(dibenzylideneacetone)dipalladium (205 mg, 0.224 mmol) and 1,1′-Bis(diphenylphosphino)ferrocene (247 mg, 0.448 mmol). The mixture was stirred for 6 h at 90° C. under nitrogen and the reaction was quenched with water (40 mL). The mixture was extracted with EA (3×100 mL). The organic layers were combined, washed with brine (2×50 mL), dried over anhydrous sodium sulfate, filtered and concentrated under reduced pressure to afford the crude product. The crude product was purified by C18 column with CH₃CN/Water (0.05% TFA), (48%). The fraction was concentrated under reduced pressure to provide 1-(t-butyl) 2,4-dimethyl 4-(2-cyanophenyl)pyrrolidine-1,2,4-tricarboxylate (470 mg, 54%) as a light yellow oil. ¹H NMR (400 MHz, DMSO-d₆) δ 7.82-8.01 (m, 1H), 7.40-7.86 (m, 3H), 4.15-4.52 (m, 2H), 3.84-4.09 (m, 1H), 3.60-3.75 (m, 6H), 2.84-3.14 (m, 2H), 1.20-1.48 (m, 9H). LC-MS (ESI, m/z): 289 [M−Boc+H]⁺.

To a stirred mixture of 1-(t-butyl) 2,4-dimethyl 4-(2-cyanophenyl)pyrrolidine-1,2,4-tricarboxylate (420 mg, 1.08 mmol) in methanol (8 mL) was added 10% palladium on activated carbon (420 mg) and HCl (0.5 mL). The mixture was stirred overnight at rt under hydrogen. The mixture was filtered through a celite pad and washed with DCM (3×30 mL). The filtrate was concentrated under reduced pressure to afford methyl 3-oxo-2,3-dihydro-1H-spiro[isoquinoline-4,3′-pyrrolidine]-5′-carboxylate (245 mg, crude) as a yellow solid. LC-MS (ESI, m/z): 261 [M+H]⁺.

A mixture of methyl 3-oxo-2,3-dihydro-1H-spiro[isoquinoline-4,3′-pyrrolidine]-5′-carboxylate (245 mg, 0.942 mmol) in ammonia (5 mL, 7 M in methanol) was stirred overnight at 50° C. and then concentrated under reduced pressure to afford 3-oxo-2,3-dihydro-1H-spiro[isoquinoline-4,3′-pyrrolidine]-5′-carboxamide (220 mg, crude) as a yellow solid. LC-MS (ESI, m/z): 513 [2M+Na]⁺.

To a mixture of 3-oxo-2,3-dihydro-1H-spiro[isoquinoline-4,3′-pyrrolidine]-5′-carboxamide (220 mg, 0.898 mmol) in CH₂Cl₂ (5 mL) were added triethylamine (273 mg, 2.69 mmol) and di-t-butyl dicarbonate (393 mg, 1.80 mmol). The mixture was stirred for 1 h at rt and then concentrated under reduced pressure to afford the crude product. The crude product was chromatographed on a silica gel column with MeOH:DCM (6:94) to provide t-butyl 5′-carbamoyl-3-oxo-2,3-dihydro-1H-spiro[isoquinoline-4,3′-pyrrolidine]-1′-carboxylate (240 mg, 77%) as a yellow solid. ¹H NMR (400 MHz, DMSO-d₆) δ 8.37 (s, 1H), 7.22-7.48 (m, 4H), 6.91-7.18 (m, 2H), 4.42-4.58 (m, 1H), 4.28-4.42 (m, 1H), 3.85-4.13 (m, 2H), 3.70-3.85 (m, 1H), 2.28-2.41 (m, 2H), 1.31-1.49 (m, 9H). LC-MS (ESI, m/z): 346 [M+H]⁺.

To a stirred mixture of t-butyl 5′-carbamoyl-3-oxo-2,3-dihydro-1H-spiro[isoquinoline-4,3′-pyrrolidine]-1′-carboxylate (240 mg, 0.696 mmol) in DCM (6 mL) was added trifluoroacetic acid (2 mL). The mixture was stirred for 1 h at rt and then concentrated under reduced pressure to afford 3-oxo-2,3-dihydro-1H-spiro[isoquinoline-4,3′-pyrrolidine]-5′-carboxamide (230 mg, crude) as a yellow oil. LC-MS (ESI, m/z): 268 [M+Na]⁺.

To a stirred mixture of 3-oxo-2,3-dihydro-1H-spiro[isoquinoline-4,3′-pyrrolidine]-5′-carboxamide (170 mg, 0.696 mmol), (S)-2-((t-butoxycarbonyl)(methyl)amino)-3-cyclopropylpropanoic acid (186 mg, 0.766 mmol) and HATU (317 mg, 0.835 mmol) in DMF (4 mL) was added DIEA (540 mg, 4.18 mmol) at 0° C. The mixture was stirred for 1 h at rt and the reaction was quenched with water (10 mL). The mixture was extracted with EA (3×50 mL). The organic layers were combined, washed with brine (2×10 mL), dried over anhydrous sodium sulfate, filtered and concentrated under reduced pressure to afford the crude product. The crude product was chromatographed on a silica gel column with MeOH:DCM (7:93) to provide t-butyl ((2S)-1-(5′-carbamoyl-3-oxo-2,3-dihydro-1H-spiro[isoquinoline-4,3′-pyrrolidin]-1′-yl)-3-cyclopropyl-1-oxopropan-2-yl)(methyl)carbamate (300 mg, 90%) as a yellow solid. ¹H NMR (400 MHz, DMSO-d₆) δ 8.40 (s, 1H), 7.20-7.82 (m, 4H), 6.78-7.22 (m, 2H), 4.69-4.90 (m, 1H), 4.40-4.62 (m, 1H), 4.20-4.43 (m, 2H), 4.05-4.20 (m, 1H), 3.58-4.03 (m, 1H), 2.52-2.88 (m, 3H), 2.22-2.48 (m, 2H), 1.47-1.80 (m, 2H), 1.10-1.45 (m, 9H), 0.47-0.65 (m, 1H), 0.25-0.46 (m, 2H), −0.02-−0.21 (m, 2H). LC-MS (ESI, m/z): 246 (fragment peak).

To a stirred mixture of t-butyl ((2S)-1-(5′-carbamoyl-3-oxo-2,3-dihydro-1H-spiro[isoquinoline-4,3′-pyrrolidin]-1′-yl)-3-cyclopropyl-1-oxopropan-2-yl)(methyl)carbamate (300 mg, 0.638 mmol) in DCM (6 mL) was added trifluoroacetic acid (2 mL). The mixture was stirred for 1 h at rt and then concentrated under reduced pressure to afford 1′-((S)-3-cyclopropyl-2-(methylamino)propanoyl)-3-oxo-2,3-dihydro-1H-spiro[isoquinoline-4,3′-pyrrolidine]-5′-carboxamide (300 mg, crude) as a yellow oil. LC-MS (ESI, m/z): 371 [M+H]⁺.

To a stirred mixture of 1′-((S)-3-cyclopropyl-2-(methylamino)propanoyl)-3-oxo-2,3-dihydro-1H-spiro[isoquinoline-4,3′-pyrrolidine]-5′-carboxamide (236 mg, 0.638 mmol), (S)-2-((t-butoxycarbonyl)amino)-3-cyclopropylpropanoic acid (161 mg, 0.702 mmol) and HATU (291 mg, 0.766 mmol) in dimethylformamide (6 mL) was added DIEA (495 mg, 3.83 mmol) at 0° C. The mixture was stirred for 2 h at rt and the reaction was quenched with water (10 mL). The mixture was extracted with EA (3×50 mL). The organic layers were combined, washed with brine (2×10 mL), dried over anhydrous sodium sulfate, filtered and concentrated under reduced pressure to afford the crude product. The crude product was chromatographed on a silica gel column with MeOH:DCM (8:92) to provide t-butyl ((2S)-1-(((2S)-1-(5′-carbamoyl-3-oxo-2,3-dihydro-1H-spiro[isoquinoline-4,3′-pyrrolidin]-1′-yl)-3-cyclopropyl-1-oxopropan-2-yl)(methyl)amino)-3-cyclopropyl-1-oxopropan-2-yl)carbamate (330 mg, 88%) as a yellow solid. ¹H NMR (400 MHz, DMSO-d₆) δ 8.40 (s, 1H), 7.15-7.86 (m, 4H), 6.55-7.18 (m, 3H), 4.98-5.45 (m, 1H), 4.19-4.78 (m, 4H), 3.57-4.18 (m, 2H), 2.58-3.17 (m, 3H), 2.02-2.47 (m, 2H), 1.45-1.70 (m, 4H), 1.30-1.40 (m, 9H), 0.54-0.83 (m, 2H), 0.22-0.53 (m, 4H), −0.02-0.26 (m, 4H). LC-MS (ESI, m/z): 604 [M+Na]⁺.

To a stirred mixture of t-butyl ((2S)-1-(((2S)-1-(5′-carbamoyl-3-oxo-2,3-dihydro-1H-spiro[isoquinoline-4,3′-pyrrolidin]-1′-yl)-3-cyclopropyl-1-oxopropan-2-yl)(methyl)amino)-3-cyclopropyl-1-oxopropan-2-yl)carbamate (330 mg, 0.568 mmol) in DCM (6 mL) was added trifluoroacetic acid (2 mL). The mixture was stirred for 1 h at rt and then concentrated under reduced pressure to afford 1′-((S)-2-((S)-2-amino-3-cyclopropyl-N-methylpropanamido)-3-cyclopropylpropanoyl)-3-oxo-2,3-dihydro-1H-spiro[isoquinoline-4,3′-pyrrolidine]-5′-carboxamide (330 mg, crude) as a yellow oil. LC-MS (ESI, m/z): 482 [M+H]⁺.

To a mixture of 1′-((S)-2-((S)-2-amino-3-cyclopropyl-N-methylpropanamido)-3-cyclopropylpropanoyl)-3-oxo-2,3-dihydro-1H-spiro[isoquinoline-4,3′-pyrrolidine]-5′-carboxamide (273 mg, 0.568 mmol) in methanol (6 mL) were added triethylamine (711 mg, 6.82 mmol) and ethyl 2,2,2-trifluoroacetate (835 mg, 5.68 mmol,) at rt. The mixture was stirred overnight and the reaction was quenched with water (10 mL). The mixture was concentrated under reduced pressure to remove MeOH and acidified to pH=5 with HCl (1 M). The mixture was extracted with EA (3×50 mL). The organic layers were combined, washed with brine (2×20 mL), dried over anhydrous sodium sulfate, filtered and concentrated under reduced pressure to afford the crude product that was chromatographed on a silica gel column with MeOH:DCM (9:91) to provide 1′-((S)-3-cyclopropyl-2-((S)-3-cyclopropyl-N-methyl-2-(2,2,2-trifluoroacetamido)propanamido)propanoyl)-3-oxo-2,3-dihydro-1H-spiro[isoquinoline-4,3′-pyrrolidine]-5′-carboxamide (290 mg, 88%) as a yellow solid. ¹H NMR (400 MHz, DMSO-d₆) δ 9.65-9.88 (m, 1H), 9.05-9.20 (m, 1H), 8.32-8.56 (m, 1H), 7.22-7.47 (m, 4H), 6.91-7.05 (m, 1H), 5.18-5.46 (m, 1H), 4.65-4.92 (m, 1H), 4.45-4.59 (m, 1H), 4.08-4.35 (m, 2H), 3.93-4.09 (m, 1H), 3.69-3.78 (m, 1H), 3.15-3.34 (m, 3H), 2.92-3.02 (m, 2H), 2.10-2.49 (m, 2H), 1.39-1.25 (m, 2H), 1.20-1.41 (m, 2H), 0.26-0.82 (m, 4H), −0.12-0.25 (m, 4H). LC-MS (ESI, m/z): 600 [M+Na]⁺.

To a solution of 1′-((S)-3-cyclopropyl-2-((S)-3-cyclopropyl-N-methyl-2-(2,2,2-trifluoroacetamido)propanamido)propanoyl)-3-oxo-2,3-dihydro-1H-spiro[isoquinoline-4,3′-pyrrolidine]-5′-carboxamide (290 mg, 0.502 mmol) in DCM (5 mL) were added pyridine (198 mg, 2.51 mmol) and trifluoroacetic anhydride (211 mg, 1.00 mmol). The mixture was stirred 1 h at rt and the reaction was quenched with water (10 mL). The mixture was extracted with DCM (3×30 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:9; Rf=0.4; detection: UV) to provide the crude product. The crude product was purified by prep-SFC (Column: Lux 5 um Cellulose-4 30*250 mm, 5.0 um; Mobile Phase A: CO₂, Mobile Phase B: ACN; Flow rate: 90 mL/min; Gradient: isocratic 42% B; Column Temperature(° C.): 35; Back Pressure(bar): 100; Wave Length: 220 nm; RT1(min): 5.04; RT2(min): 10.28; Sample Solvent: ACN; Injection Volume: 1 mL; Number Of Runs: 6) to provide the desired crude product (S)—N—((S)-1-((4R,5'S)-5′-cyano-3-oxo-2,3-dihydro-1H-spiro[isoquinoline-4,3′-pyrrolidin]-1′-yl)-3-cyclopropyl-1-oxopropan-2-yl)-3-cyclopropyl-N-methyl-2-(2,2,2-trifluoroacetamido)propanamide (first peak, 20 mg) and (S)—N—((S)-1-((4S,5′R)-5′-cyano-3-oxo-2,3-dihydro-1H-spiro[isoquinoline-4,3′-pyrrolidin]-1′-yl)-3-cyclopropyl-1-oxopropan-2-yl)-3-cyclopropyl-N-methyl-2-(2,2,2-trifluoroacetamido)propanamide (second peak, 37 mg).

The first peak was purified twice by prep-HPLC (Column: Xselect CSH Prep OBD C18 Column 30*150 mm, 5 m; Mobile Phase A: Water (H₂O), Mobile Phase B: ACN; Flow rate: 60 mL/min; Gradient: 31% B to 61% B in 10 min; Wave Length: 220 nm; RT1(min): 8.58) to provide (S)—N—((S)-1-((4R,5'S)-5′-cyano-3-oxo-2,3-dihydro-1H-spiro[isoquinoline-4,3′-pyrrolidin]-1′-yl)-3-cyclopropyl-1-oxopropan-2-yl)-3-cyclopropyl-N-methyl-2-(2,2,2-trifluoroacetamido)propanamide (9.7 mg, 3%) as a white solid. ¹H NMR (400 MHz, 80° C., DMSO-d₆) δ 9.35 (br, 1H), 8.21 (br, 1H), 7.29-7.32 (m, 2H), 7.21-7.27 (m, 1H), 7.08-7.18 (m, 1H), 5.30 (br, 1H), 4.85 (br, 1H), 4.70 (br, 1H), 4.41-4.52 (m, 1H), 4.29-4.40 (m, 1H), 4.13-4.27 (m, 1H), 3.92-4.08 (m, 1H), 2.87-2.96 (m, 3H), 2.71-2.86 (m, 1H), 2.58-2.69 (m, 1H), 1.55-1.78 (m, 2H), 1.32-1.55 (m, 2H), 0.51-0.72 (m, 2H), 0.22-0.48 (m, 4H), 0.04-0.19 (m, 3H), −0.02-0.03 (m, 1H). LC-MS (ESI, m/z): 582 [M+Na]⁺.

The second peak was purified twice by prep-HPLC (Column: Xselect CSH Prep OBD C18 Column 30*150 mm, 5 m; Mobile Phase A: Water (H₂O), Mobile Phase B: ACN; Flow rate: 60 mL/min; Gradient: 32% B to 62% B in 10 min; Wave Length: 220 nm; RT1(min): 8.53) to provide (S)—N—((S)-1-((4S,5′R)-5′-cyano-3-oxo-2,3-dihydro-1H-spiro[isoquinoline-4,3′-pyrrolidin]-1′-yl)-3-cyclopropyl-1-oxopropan-2-yl)-3-cyclopropyl-N-methyl-2-(2,2,2-trifluoroacetamido)propanamide (19.6 mg, 6%) as a white solid. ¹H NMR (400 MHz, 80° C., DMSO-d₆) δ 9.41 (br, 1H), 8.23 (br, 1H), 7.23-7.38 (m, 3H), 7.09-7.22 (m, 1H), 5.21-5.29 (m, 1H), 4.91-5.16 (m, 1H), 4.71-4.90 (m, 1H), 4.41-4.52 (m, 1H), 4.29-4.40 (m, 1H), 3.84-3.99 (m, 2H), 2.91-3.01 (m, 3H), 2.82-2.90 (m, 1H), 2.57-2.68 (m, 1H), 1.48-1.77 (m, 3H), 1.21-1.45 (m, 1H), 0.56-0.80 (m, 1H), 0.31-0.53 (m, 3H), 0.13-0.28 (m, 3H), 0.02-0.12 (m, 1H), −0.13-−0.03 (m, 2H). LC-MS (ESI, m/z): 582 [M+Na]⁺.

### Example 69

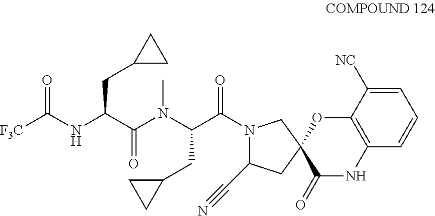 [SMILES: CN(C(=O)[C@H](CC1CC1)NC(=O)C(F)(F)F)[C@@H](CC1CC1)C(=O)N1C[C@@]2(CC1C#N)Oc1c(C#N)cccc1NC2=O]

t-butyl (2R,5'S)-8-bromo-5′-carbamoyl-3-oxo-3,4-dihydrospiro[benzo[b][1,4]oxazine-2,3′-pyrrolidine]-1′-carboxylate was made similarly as described for (2R,5'S)-6-fluoro-3-oxo-3,4-dihydrospiro[benzo[b][1,4]oxazine-2,3′-pyrrolidine]-5′-carboxamide hydrochloride except that 2-amino-6-bromophenol was used instead of 2-amino-4-fluorophenol.

A solution of t-butyl (2R,5'S)-8-bromo-5′-carbamoyl-3-oxo-3,4-dihydrospiro[benzo[b][1,4]oxazine-2,3′-pyrrolidine]-1′-carboxylate (220 mg, 0.520 mmol), zinc cyanide (72.7 mg, 0.620 mmol), tris(dibenzylideneacetone) dipalladium (47.3 mg, 0.05 mmol), 1,1′-Bis(diphenylphosphino)ferrocene (57.0 mg, 0.104 mmol) and zinc powder (23.6 mg, 0.360 mmol) in DMF (5 mL) under nitrogen at rt was stirred for 3 h at 120° C. under nitrogen and the reaction was quenched with water (15 mL). The mixture was extracted with EA (3×20 mL). The organic layers were combined, washed with brine (2×20 mL), dried over anhydrous sodium sulfate, filtered and concentrated under reduced pressure to afford the crude product. The crude product was chromatographed on a silica gel column with MeOH:DCM (1:19) to provide t-butyl(2R,5'S)-5′-carbamoyl-8-cyano-3-oxo-3,4-dihydrospiro[benzo[b][1,4]oxazine-2,3′-pyrrolidine]-1′-carboxylate (100 mg, 52%) as a yellow solid. ¹H NMR (400 MHz, DMSO-d₆) δ 11.29 (s, 1H), 7.36-7.66 (m, 4H), 7.02-7.12 (m, 1H), 4.20-4.33 (m, 1H), 3.77-3.82 (m, 1H), 3.55-3.73 (m, 1H), 2.53-2.61 (m, 1H), 2.30-2.44 (m, 1H), 1.33-1.42 (m, 9H). LC-MS (ESI, m/z): 317 [M-56]⁺.

To a solution of t-butyl (2R,5'S)-5′-carbamoyl-8-cyano-3-oxo-3,4-dihydrospiro[benzo[b][1,4]oxazine-2,3′-pyrrolidine]-1′-carboxylate (90.0 mg, 0.240 mmol) in DCM (3 mL) was added TFA (1 mL). The mixture was stirred for 1 h at rt and then concentrated under reduced pressure to afford (2R,5'S)-8-cyano-3-oxo-3,4-dihydrospiro[benzo[b][1,4]oxazine-2,3′-pyrrolidine]-5′-carboxamide (65 mg, crude) as a yellow solid. LC-MS (ESI, m/z): 273 [M+H]⁺.

To a solution of (2R,5'S)-8-cyano-3-oxo-3,4-dihydrospiro[benzo[b][1,4]oxazine-2,3′-pyrrolidine]-5′-carboxamide (65.0 mg, 0.240 mmol), (2R,5'S)-8-cyano-3-oxo-3,4-dihydrospiro[benzo[b][1,4]oxazine-2,3′-pyrrolidine]-5′-carboxamide (59.0 mg, 0.240 mmol) and HATU (109 mg, 0.290 mmol) in DMF (2 mL) was added DIEA (154 mg, 1.20 mmol) at 0° C. The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product which was chromatographed on a silica gel column with MeOH:DCM (1:16) to provide t-butyl ((S)-1-((2R,5'S)-5′-carbamoyl-8-cyano-3-oxo-3,4-dihydrospiro[benzo[b][1,4]oxazine-2,3′-pyrrolidin]-1′-yl)-3-cyclopropyl-1-oxopropan-2-yl)(methyl)carbamate (65 mg, 54%) as a yellow solid. ¹H NMR (400 MHz, DMSO-d₆) δ 11.40 (s, 1H), 7.58 (s, 1H), 7.36-7.47 (m, 1H), 7.13-7.26 (m, 2H), 7.03 (s, 1H), 4.60-4.81 (m, 1H), 4.38-4.47 (m, 1H), 3.97-4.08 (m, 1H), 3.82-3.94 (m, 1H), 3.16 (s, 3H), 2.54-4.61 (m, 1H), 2.38-2.47 (m, 1H), 1.14-1.44 (m, 11H), 0.46-0.56 (m, 1H), 0.27-0.44 (m, 2H), 0.006-0.17 (m, 2H). LC-MS (ESI, m/z): 520 [M+Na]⁺.

To a solution of t-butyl ((S)-1-((2R,5'S)-5′-carbamoyl-8-cyano-3-oxo-3,4-dihydrospiro[benzo[b][1,4]oxazine-2,3′-pyrrolidin]-1′-yl)-3-cyclopropyl-1-oxopropan-2-yl)(methyl)carbamate (60.0 mg, 0.121 mmol) in DCM (3 mL) and TFA (1 mL). The mixture was stirred for 1 h at rt and then concentrated under reduced pressure to afford (2R,5'S)-8-cyano-1′-((S)-3-cyclopropyl-2-(methylamino)propanoyl)-3-oxo-3,4-dihydrospiro[benzo[b][1,4]oxazine-2,3′-pyrrolidine]-5′-carboxamide (48 mg crude) as a yellow solid. LC-MS (ESI, m/z): 398 [M+H]⁺.

To a solution of (2R,5'S)-8-cyano-1′-((S)-3-cyclopropyl-2-(methylamino)propanoyl)-3-oxo-3,4-dihydrospiro[benzo[b][1,4]oxazine-2,3′-pyrrolidine]-5′-carboxamide (48.0 mg, 0.120 mmol), (S)-2-((t-butoxycarbonyl)amino)-3-cyclopropylpropanoic acid (27.7 mg, 0.120 mmol) and HATU (55.0 mg, 0.145 mmol) in DMF (2 mL) was added DIEA(78.0 mg, 0.600 mmol) at 0° C. The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM)=1:10; Rf=0.3; detection: UV) to provide t-butyl ((S)-1-(((S)-1-((2R,5'S)-5′-carbamoyl-8-cyano-3-oxospiro[chromane-2,3′-pyrrolidin]-1′-yl)-3-cyclopropyl-1-oxopropan-2-yl)(methyl)amino)-3-cyclopropyl-1-oxopropan-2-yl)carbamate. ¹H NMR (400 MHz, DMSO-d₆) δ 11.37 (s, 1H), 7.38-7.57 (m, 2H), 7.14-7.29 (m, 2H), 6.95-7.08 (m, 2H), 5.23-5.34 (m, 1H), 4.34-4.42 (m, 1H), 3.97-4.15 (m, 2H), 2.84-3.00 (m, 3H), 2.55-2.65 (m, 1H), 1.98-2.09 (m, 1H), 1.27-1.66 (m, 13H), 0.56-0.71 (m, 2H), 0.16-0.47 (m, 4H), 0.003-0.07 (m, 2H), −0.21-−0.11 (m, 1H), −0.39-−0.30 (m, 1H). LC-MS (ESI, m/z): 620 [M+Na]⁺.

To a solution of t-butyl ((S)-1-(((S)-1-((2R,5'S)-5′-carbamoyl-8-cyano-3-oxo-3,4-dihydrospiro[benzo[b] [1,4]oxazine-2,3′-pyrrolidin]-1′-yl)-3-cyclopropyl-1-oxopropan-2-yl)(methyl)amino)-3-cyclopropyl-1-oxopropan-2-yl)carbamate (60.0 mg, 0.100 mmol) in DCM (3 mL) was added TFA (1 mL). The mixture stirred for 1 h at rt and then was concentrated under reduced pressure to afford (2R,5'S)-1′-((S)-2-((S)-2-amino-3-cyclopropyl-N-methylpropanamido)-3-cyclopropylpropanoyl)-8-cyano-3-oxo-3,4-dihydrospiro[benzo[b][1,4]oxazine-2,3′-pyrrolidine]-5′-carboxamide (50 mg, crude) as a yellow solid. LC-MS (ESI, m/z): 509 [M+H]⁺.

To a solution of (2R,5'S)-1′-((S)-2-((S)-2-amino-3-cyclopropyl-N-methylpropanamido)-3-cyclopropylpropanoyl)-8-cyano-3-oxo-3,4-dihydrospiro[benzo[b][1,4]oxazine-2,3′-pyrrolidine]-5′-carboxamide (50.0 mg, 0.098 mmol) in MeOH (2 mL) was added triethylamine (120 mg, 1.18 mmol) and ethyl 2,2,2-trifluoroacetate (139 mg, 0.980 mmol). The mixture was stirred overnight at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product which was purified by TLC (Mobile phase: (EA) Rf=0.2; detection: UV) to provide (2R,5'S)-8-cyano-1′-((S)-3-cyclopropyl-2-((S)-3-cyclopropyl-N-methyl-2-(2,2,2-trifluoroacetamido)propanamido)propanoyl)-3-oxo-3,4-dihydrospiro[benzo[b][1,4]oxazine-2,3′-pyrrolidine]-5′-carboxamide (36 mg, crude) as a yellow solid. LC-MS (ESI, m/z): 627 [M+Na]⁺.

To a solution of (2R,5'S)-8-cyano-1′-[(2S)-3-cyclopropyl-2-[(2S)-3-cyclopropyl-N-methyl-2-(2,2,2-trifluoroacetamido)propanamido]propanoyl]-3-oxo-4H-spiro[1,4-benzoxazine-2,3′-pyrrolidine]-5′-carboxamide (36.0 mg, 0.06 mmol) in DCM (2 mL) were added pyridine (23.6 mg, 0.300 mmol) and trifluoroacetic anhydride (25.0 mg, 0.120 mmol). The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was purified by prep-HPLC (Column: Xselect CSH Prep C18 Column, 30*150 mm, 5 m; Mobile Phase A: Water (0.1% FA), Mobile Phase B: ACN; Flow rate: 60 mL/min; Gradient: 40% B to 61% B in 10 min; Wave Length: 254 nm/220 nm; RT1(min): 8.68) to provide (2S)—N-[(2S)-1-[(2R,5'S)-8-cyano-5′-ethynyl-3-oxo-4H-spiro[1,4-benzoxazine-2,3′-pyrrolidin]-1′-yl]-3-cyclopropyl-1-oxopropan-2-yl]-3-cyclopropyl-N-methyl-2-(2,2,2-trifluoroacetamido)propanamide (8.3 mg, 23%) as a white solid. ¹H NMR (400 MHz, 80° C. DMSO-d₆) δ 9.35 (s, 1H), 7.35-7.41 (m, 1H), 7.20-7.30 (m, 1H), 7.11-7.19 (m, 1H), 5.25-5.35 (m, 1H), 4.81-4.92 (m, 1H), 4.65-4.82 (m, 1H), 4.08-4.40 (m, 2H), 2.97-3.05 (m, 3H), 2.80-2.90 (m, 1H), 2.25-2.60 (m, 1H), 1.61-1.80 (m, 2H), 1.40-1.60 (m, 2H), 0.51-0.72 (m, 2H), 0.30-0.48 (m, 2H), 0.20-0.45 (m, 2H), 0.03-0.15 (m, 2H), −0.80-−0.02 (m, 2H). LC-MS (ESI, m/z): 609 [M+Na]⁺.

### Example 70

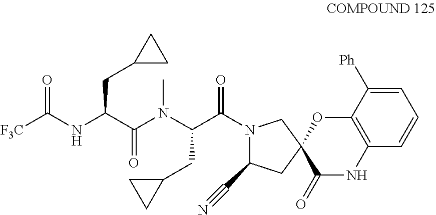 [SMILES: CN(C(=O)[C@H](CC1CC1)NC(=O)C(F)(F)F)[C@@H](CC1CC1)C(=O)N1C[C@@]2(C[C@H]1C#N)Oc1c(cccc1-c1ccccc1)NC2=O]

To a mixture of t-butyl (2R,5'S)-8-bromo-5′-carbamoyl-3-oxo-3,4-dihydrospiro[benzo[b][1,4]oxazine-2,3′-pyrrolidine]-1′-carboxylate (200 mg, 0.469 mmol) in toluene (3.5 mL) and ethanol (0.75 mL) were added tetrakis(triphenylphosphine)palladium (10.8 mg, 0.009 mmol) and potassium carbonate (195 mg, 1.41 mmol) in water (0.75 mL). The mixture was stirred overnight at 110° C. under nitrogen and the reaction was quenched with water (10 mL). The mixture was extracted with EA (3×50 mL). The organic layers were combined, washed with brine (2×10 mL), dried over anhydrous sodium sulfate, filtered and concentrated under reduced pressure to afford the crude product. The crude product was purified by C18 column with CH₃CN:Water (0.05% TFA), (42%). The fraction was concentrated under reduced pressure to provide t-butyl (2R,5'S)-5′-carbamoyl-3-oxo-8-phenyl-3,4-dihydrospiro[benzo[b][1,4]oxazine-2,3′-pyrrolidine]-1′-carboxylate (90 mg, 45%) as a yellow oil. ¹H NMR (400 MHz, DMSO-d₆) δ 11.04 (s, 1H), 7.56-7.62 (m, 1H), 7.35-7.54 (m, 5H), 6.84-7.20 (m, 4H), 4.13-4.29 (m, 1H), 3.77-3.89 (m, 1H), 3.48-3.61 (m, 1H), 2.46-2.49 (m, 2H), 1.33-1.49 (m, 9H). LC-MS (ESI, m/z): 324 [M−Boc+H]⁺.

The remainder of the synthesis was analogous to the synthesis of (2S)—N-[(2S)-1-[(2R,5'S)-8-cyano-5′-ethynyl-3-oxo-4H-spiro[1,4-benzoxazine-2,3′-pyrrolidin]-1′-yl]-3-cyclopropyl-1-oxopropan-2-yl]-3-cyclopropyl-N-methyl-2-(2,2,2-trifluoroacetamido)propenamide. The crude product was purified by TLC (Mobile phase: MeOH/DCM=1:9; Rf=0.4; detection: UV) to provide the desired crude product. The crude product was purified by prep-HPLC (Column: Xselect CSH Prep OBD C18, 30*150 mm, 5 m; Mobile Phase A: Water (0.1% FA), Mobile Phase B: ACN; Flow rate: 60 mL/min; Gradient: 31% B to 70% B in 10 min; Wave Length: 220 nm; RT(min): 8.68 to provide (S)—N—((S)-1-((2R,5'S)-5′-cyano-3-oxo-8-phenyl-3,4-dihydrospiro[benzo[b][1,4]oxazine-2,3′-pyrrolidin]-1′-yl)-3-cyclopropyl-1-oxopropan-2-yl)-3-cyclopropyl-N-methyl-2-(2,2,2-trifluoroacetamido)propanamide (14.6 mg, 23%) as a white solid. ¹H NMR (400 MHz, 80° C., DMSO-d₆) δ 10.53-11.59 (m, 1H), 8.72-9.82 (m, 1H), 7.26-7.52 (m, 5H), 7.08-7.21 (m, 1H), 6.91-7.07 (m, 2H), 5.11-5.32 (m, 1H), 4.36-4.96 (m, 2H), 3.81-4.29 (m, 2H), 2.84-2.93 (m, 1H), 2.58-2.74 (m, 4H), 1.41-1.89 (m, 2H), 1.28-1.42 (m, 1H), 1.03-1.26 (m, 1H), 0.42-0.78 (m, 2H), 0.17-0.47 (m, 4H), −0.03-0.16 (m, 2H), −0.18-−0.03 (m, 1H), −0.42-−0.19 (m, 1H). LC-MS (ESI, m/z): 660 [M+Na]⁺.

### Example 71

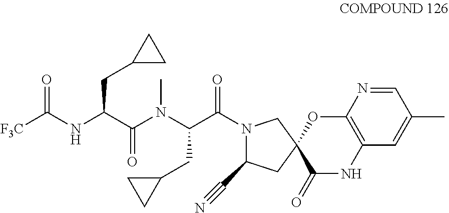 [SMILES: Cc1cnc2c(c1)NC(=O)[C@]1(C[C@@H](C#N)N(C(=O)[C@H](CC3CC3)N(C)C(=O)[C@H](CC3CC3)NC(=O)C(F)(F)F)C1)O2]

To a mixture of 1-(t-butyl) 2,4-dimethyl (2S)-4-hydroxypyrrolidine-1,2,4-tricarboxylate (1.0 g, 3.30 mmol) in tetrahydrofuran (25 mL) was added sodium bis(trimethylsilyl)amide (2.6 mL) at −78° C. and the mixture was stirred for 5 min. 2-fluoro-5-methyl-3-nitropyridine (824 mg, 5.28 mmol) in tetrahydrofuran (5 mL) was added. The mixture was stirred for 6 h and the reaction was quenched with aqueous ammonium chloride (80 mL). The mixture was extracted with EA (3×100 mL). The organic layers were combined, washed with brine (2×20 mL), dried over anhydrous sodium sulfate, filtered and concentrated under reduced pressure to afford the crude product. The crude product was chromatographed on a silica gel column with EA:PE (33:67) to provide 1-(t-butyl) 2,4-dimethyl (2S)-4-((5-methyl-3-nitropyridin-2-yl)oxy)pyrrolidine-1,2,4-tricarboxylate (500 mg, 34%) as a yellow oil. ¹H NMR (400 MHz, DMSO-d₆) δ 8.12-8.51 (m, 2H), 4.36-4.62 (m, 1H), 4.17-4.26 (m, 1H), 3.78-4.00 (m, 1H), 3.56-3.76 (m, 6H), 2.77-2.98 (m, 1H), 2.53-2.70 (m, 1H), 2.24-2.38 (m, 3H), 1.33-1.42 (m, 9H). LC-MS (ESI, m/z): 440 [M+H]⁺.

To mixture of 1-(t-butyl) 2,4-dimethyl (2S)-4-((5-methyl-3-nitropyridin-2-yl)oxy)pyrrolidine-1,2,4-tricarboxylate (490 mg, 1.115 mmol) in methanol (5 mL) and H₂O (1.25 mL), were added iron (311 mg, 5.58 mmol) and ammonium chloride (143 mg, 2.68 mmol). The mixture was stirred for overnight at 60° C. The mixture was filtered and the filter cake was washed with methanol (2×20 mL). The mixture was concentrated under reduced pressure and then diluted with water (30 mL). The mixture was extracted with EA (3×30 mL). The organic layers were combined, washed with brine (2×10 mL), dried over anhydrous sodium sulfate, filtered and concentrated under reduced pressure to afford 1′-(t-butyl) 5′-methyl (5'S)-7-methyl-2-oxo-1,2-dihydrospiro[pyrido[2,3-b][1,4]oxazine-3,3′-pyrrolidine]-1′,5′-dicarboxylate (400 mg, 95%) as a light yellow solid. ¹H NMR (400 MHz, DMSO-d₆) δ 10.91-11.22 (m, 1H), 7.54-7.78 (m, 1H), 6.94-7.22 (m, 1H), 4.29-4.67 (m, 1H), 3.85-4.10 (m, 1H), 3.66-3.73 (m, 3H), 3.54-3.66 (m, 1H), 2.60-2.81 (m, 1H), 2.26-2.48 (m, 1H), 2.19-2.25 (m, 3H), 1.32-1.41 (m, 9H). LC-MS (ESI, m/z): 378 [M+H]⁺.

To a stirred mixture of 1′-(t-butyl) 5′-methyl (5'S)-7-methyl-2-oxo-1,2-dihydrospiro[pyrido[2,3-b][1,4]oxazine-3,3′-pyrrolidine]-1′,5′-dicarboxylate (400 mg, 1.06 mmol) was added NH₃(gas) in methanol (15 mL) in a sealed tube. The mixture was stirred for overnight at 50° C. and then concentrated under reduced pressure to provide t-butyl (5'S)-5′-carbamoyl-7-methyl-2-oxo-1,2-dihydrospiro[pyrido[2,3-b][1,4]oxazine-3,3′-pyrrolidine]-1′-carboxylate (300 mg, crude) as a yellow solid. LC-MS (ESI, m/z): 363 [M+H]⁺.

To a stirred mixture of t-butyl (5'S)-5′-carbamoyl-7-methyl-2-oxo-1,2-dihydrospiro[pyrido[2,3-b][1,4]oxazine-3,3′-pyrrolidine]-1′-carboxylate (180 mg, 0.497 mmol) in CH₂Cl₂ (3 mL) was added trifluoroacetic acid (1 mL). The mixture was stirred for 1 h at rt and then concentrated under reduced pressure to (5'S)-7-methyl-2-oxo-1,2-dihydrospiro[pyrido[2,3-b][1,4]oxazine-3,3′-pyrrolidine]-5′-carboxamide (130 mg, crude) as a light yellow semi-solid. LC-MS (ESI, m/z): 263 [M+H]⁺.

To a mixture of (5'S)-7-methyl-2-oxo-1,2-dihydrospiro[pyrido[2,3-b][1,4]oxazine-3,3′-pyrrolidine]-5′-carboxamide (130 mg, 0.496 mmol), (S)-2-((t-butoxycarbonyl)(methyl)amino)-3-cyclopropylpropanoic acid (121 mg, 0.496 mmol) and HATU (226 mg, 0.595 mmol) in DMF(3 mL) was added N-ethyl-N-isopropylpropan-2-amine (DIEA) (384 mg, 2.98 mmol) at 0° C. The mixture was stirred for 1 h at rt and the reaction was quenched with water (20 mL). The mixture was extracted with EA (3×50 mL). The organic layers were combined, washed with brine (2×10 mL), dried over anhydrous sodium sulfate, filtered and concentrated under reduced pressure to afford the crude product, purified by prep-TLC (MeOH:DCM=1:10 to afford t-butyl ((2S)-1-((5'S)-5′-carbamoyl-7-methyl-2-oxo-1,2-dihydrospiro[pyrido[2,3-b][1,4]oxazine-3,3′-pyrrolidin]-1′-yl)-3-cyclopropyl-1-oxopropan-2-yl)(methyl)carbamate (170 mg, 70%) as a light yellow solid. ¹H NMR (400 MHz, DMSO-d₆) δ 10.87-11.23 (m, 1H), 7.59-7.81 (m, 1H), 7.30-7.52 (m, 1H), 6.99-7.13 (m, 2H), 4.56-4.92 (m, 1H), 4.31-4.48 (m, 1H), 3.71-4.20 (m, 2H), 2.66-2.95 (m, 1H), 2.54-2.64 (m, 3H), 2.30-2.47 (m, 1H), 2.22 (s, 3H), 1.47-1.70 (m, 1H), 1.14-1.45 (m, 10H), 0.46-0.61 (m, 1H), 0.26-0.44 (m, 2H), 0.01-0.12 (m, 2H). LC-MS (ESI, m/z): 488 [M+H]⁺.

To a mixture of t-butyl ((2S)-1-((5'S)-5′-carbamoyl-7-methyl-2-oxo-1,2-dihydrospiro[pyrido[2,3-b][1,4]oxazine-3,3′-pyrrolidin]-1′-yl)-3-cyclopropyl-1-oxopropan-2-yl)(methyl)carbamate (170 mg, 0.349 mmol) in CH₂Cl₂ (3 mL) was added TFA (1 mL). The mixture was stirred for 1 h at rt and then concentrated under reduced pressure to afford (5'S)-1′-((S)-3-cyclopropyl-2-(methylamino)propanoyl)-7-methyl-2-oxo-1,2-dihydrospiro[pyrido[2,3-b][1,4]oxazine-3,3′-pyrrolidine]-5′-carboxamide (135 mg, crude) as yellow semi-solid. LC-MS (ESI, m/z): 388 [M+H]⁺.

To a stirred mixture of (5'S)-1′-((S)-3-cyclopropyl-2-(methylamino)propanoyl)-7-methyl-2-oxo-1,2-dihydrospiro[pyrido[2,3-b][1,4]oxazine-3,3′-pyrrolidine]-5′-carboxamide (135 mg, 0.348 mmol), (S)-2-((t-butoxycarbonyl)amino)-3-cyclopropylpropanoic acid (87.9 mg, 0.348 mmol) and HATU (159 mg, 0.418 mmol) in DMF (3 mL) was added DIEA (270 mg, 2.09 mmol) at 0° C. The mixture was stirred for 1 h at rt and the reaction was quenched with water (20 mL). The mixture was extracted with EA (3×50 mL). The organic layers were combined, washed with brine (2×10 mL), dried over anhydrous sodium sulfate, filtered and concentrated under reduced pressure to afford the crude product, which was chromatographed on a silica gel column with MeOH:CH₂Cl₂ (5:95) to provide t-butyl ((2S)-1-(((2S)-1-((5'S)-5′-carbamoyl-7-methyl-2-oxo-1,2-dihydrospiro[pyrido[2,3-b][1,4]oxazine-3,3′-pyrrolidin]-1′-yl)-3-cyclopropyl-1-oxopropan-2-yl)(methyl)amino)-3-cyclopropyl-1-oxopropan-2-yl)carbamate (180 mg, crude) as a light yellow solid. LC-MS (ESI, m/z): 599 [M+H]⁺.

To a stirred mixture of t-butyl ((2S)-1-(((2S)-1-((5'S)-5′-carbamoyl-7-methyl-2-oxo-1,2-dihydrospiro[pyrido[2,3-b][1,4]oxazine-3,3′-pyrrolidin]-1′-yl)-3-cyclopropyl-1-oxopropan-2-yl)(methyl)amino)-3-cyclopropyl-1-oxopropan-2-yl)carbamate (180 mg, 0.301 mmol) in CH₂Cl₂ (3 mL) was added trifluoroacetic acid (1 mL). The mixture was stirred for 1 h at rt and then concentrated under reduced pressure to provide (5'S)-1′-((S)-2-((S)-2-amino-3-cyclopropyl-N-methylpropanamido)-3-cyclopropylpropanoyl)-7-methyl-2-oxo-1,2-dihydrospiro[pyrido[2,3-b][1,4]oxazine-3,3′-pyrrolidine]-5′-carboxamide (149 mg, crude) as a light yellow semi-solid. LC-MS (ESI, m/z): 499 [M+H]⁺.

To a stirred mixture of (5'S)-1′-((S)-2-((S)-2-amino-3-cyclopropyl-N-methylpropanamido)-3-cyclopropylpropanoyl)-7-methyl-2-oxo-1,2-dihydrospiro[pyrido[2,3-b][1,4]oxazine-3,3′-pyrrolidine]-5′-carboxamide (149 mg, 0.299 mmol) in methanol (3 mL) were added trimethylamine (362 mg, 3.59 mmol) and trifluoroethyl acetate (424 mg, 2.99 mmol). The mixture was stirred for overnight at rt and the reaction was quenched with water (20 mL). The mixture was acidified to pH=3 with HCl (1M) and then extracted with EA (3×50 mL). The organic layers were combined, washed with brine (2×20 mL) and dried over anhydrous sodium sulfate. The solids were removed by filtration and the filtrate was concentrated under reduced pressure to afford crude product, purified by prep-TLC (MeOH:DCM=1:10 to afford (5'S)-1′-((S)-3-cyclopropyl-2-((S)-3-cyclopropyl-N-methyl-2-(2,2,2-trifluoroacetamido)propanamido)propanoyl)-7-methyl-2-oxo-1,2-dihydrospiro[pyrido[2,3-b][1,4]oxazine-3,3′-pyrrolidine]-5′-carboxamide (170 mg, crude) as a light yellow solid. LC-MS (ESI, m/z): 595 [M+H]⁺.

To a mixture of (5'S)-1′-((S)-3-cyclopropyl-2-((S)-3-cyclopropyl-N-methyl-2-(2,2,2-trifluoroacetamido)propanamido)propanoyl)-7-methyl-2-oxo-1,2-dihydrospiro[pyrido[2,3-b][1,4]oxazine-3,3′-pyrrolidine]-5′-carboxamide (170 mg, 0.286 mmol) in CH₂Cl₂ (3 mL) were added pyridine (113 mg, 1.43 mmol) and trifluoroacetic anhydride (120 mg, 0.572 mmol). The mixture was stirred for 1 h at rt and the reaction was quenched with water (20 mL), then extracted with CH₂Cl₂ (3×50 mL). The organic layers were combined, washed with brine (2×10 mL), dried over anhydrous sodium sulfate, the solids were removed by filtration and the filtrate was concentrated under reduced pressure to afford the crude product, which was purified by prep-HPLC (Column: DAICEL DCpak P4VP 3×25 cm, 5 m; Mobile Phase A: CO₂, Mobile Phase B: IPA(1%-2M—NH3-IPA); Flow rate: 65 mL/min; Gradient: isocratic 38% B; Column Temperature: 35C; Wave Length: 220 nm; RT(min): 4.89; Sample Solvent: ACN; Injection Volume: 1 mL) to provide (S)—N—((S)-1-((3R,5'S)-5′-cyano-7-methyl-2-oxo-1,2-dihydrospiro[pyrido[2,3-b][1,4]oxazine-3,3′-pyrrolidin]-1′-yl)-3-cyclopropyl-1-oxopropan-2-yl)-3-cyclopropyl-N-methyl-2-(2,2,2-trifluoroacetamido)propanamide (16.3 mg, 9%) as an off-white solid. ¹H NMR (400 MHz, 80° C., DMSO-d₆) δ 10.90 (s, 1H), 9.20-9.40 (m, 1H), 7.50-7.70 (m, 1H), 7.00-7.10 (m, 1H), 5.10-5.25 (m, 1H), 4.75-4.95 (m, 1H), 4.60-4.75 (m, 1H), 3.95-4.10 (m, 2H), 2.95 (s, 3H), 2.70-2.80 (m, 1H), 2.50-2.65 (m, 1H), 2.15 (s, 3H), 1.50-1.70 (m, 2H), 1.35-1.49 (m, 2H), 0.50-0.65 (m, 2H), 0.15-0.40 (m, 4H), −0.05-0.10 (m, 3H), −0.20-−0.10 (m, 1H). LC-MS (ESI, m/z): 577 [M+H]⁺.

### Example 72

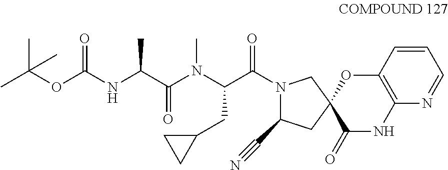 [SMILES: C[C@H](NC(=O)OC(C)(C)C)C(=O)N(C)[C@@H](CC1CC1)C(=O)N1C[C@@]2(C[C@H]1C#N)Oc1cccnc1NC2=O]

Compound 127 was made according to the procedure to make Compound 84 with the exception that the boc group remained on the alanine during synthesis to the final product. LC-MS (ESI, m/z): 527 [M+H]⁺.

The following compounds were made using analogous methods as for those described above.

| | | LC-MS | |
|---|---|---|---|
| Entry | Structure | (ESI, m/z) | NMR |
| 128 | 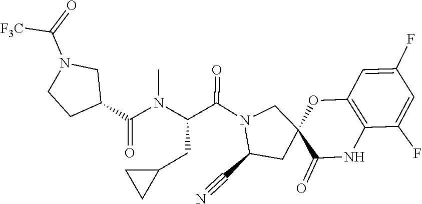 [SMILES: CN(C(=O)[C@@H]1CCN(C(=O)C(F)(F)F)C1)[C@@H](CC1CC1)C(=O)N1C[C@@]2(C[C@H]1C#N)Oc1cc(F)cc(F)c1NC2=O] | 582 [M − H]⁻ | ¹H NMR (500 MHz, 364K, DMSO-d₆) δ 10.50 (br. s., 1H), 6.91 (m, 1H), 6.70 (m, 1H), 5.15 (m, 1H), 4.96 (m, 1H), 4.11 (m, 1H), 4.00 (m, 1H), 3.36-3.81 (m, 5H), 2.99 (s, 3H), 2.78 (m, 2H), 2.01-2.17 (m, 1H), 1.55-1.98 (m, 3H), 0.60 (m, 1H), 0.38 (m, 2H), 0.09 (m, 2H) |
| 129 | 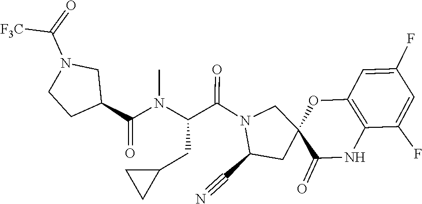 [SMILES: CN(C(=O)[C@H]1CCN(C(=O)C(F)(F)F)C1)[C@@H](CC1CC1)C(=O)N1C[C@@]2(C[C@H]1C#N)Oc1cc(F)cc(F)c1NC2=O] | 582 [M − H]⁻ | ¹H NMR (500 MHz, 363K, DMSO-d₆) δ 10.70 (br. s., 1H), 6.90 (m, 1H), 6.70 (m, 1H), 5.18 (m, 1H), 4.97 (m, 1H), 4.13 (m, 1H), 3.97 (m, 1H), 3.33-3.73 (m, 5H), 2.99 (s, 3H), 2.79 (m, 2H), 2.14-2.31 (m, 1H), 1.80-2.06 (m, 1H), 1.58-1.73 (m, 2H), 0.59 (m, 1H), 0.40 (m, 2H), 0.04-0.15 (m, 2H) |
| 130 | 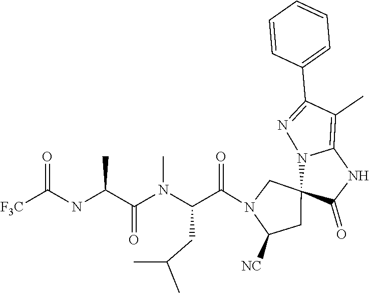 | 588 [M + H]⁺ | ¹H NMR (500 MHz, 363K, DMSO-d₆) δ 9.0-11.0 (br. s., 2H), 7.60 (m, 2H), 7.39 (m, 2H), 7.32 (m, 1H), 5.24 (m, 1H), 5.14 (m, 1H), 4.54 (m, 1H), 3.86-4.04 (m, 2H), 2.87 (s, 3H), 2.82 (m, 1H), 2.64-2.71 (m, 1H), 2.09 (s, 3H), 1.65 (m, 1H), 1.41-1.60 (m, 2H), 0.93 (m, 3H), 0.89 (d, 3H), 0.85 (d, 3H) |
| 131 | 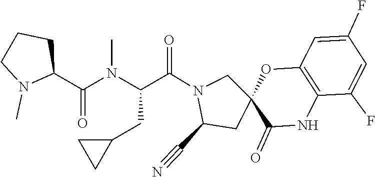 [SMILES: CN1CCC[C@H]1C(=O)N(C)[C@@H](CC1CC1)C(=O)N1C[C@@]2(C[C@H]1C#N)Oc1cc(F)cc(F)c1NC2=O] | 502 [M + H]⁺ | ¹H NMR (500 MHz, 363K, DMSO-d₆) δ 11.00 (br. s., 1H), 6.93 (m, 1H), 6.70 (m, 1H), 5.19 (m, 1H), 4.95 (m, 1H), 3.96-4.12 (m, 2H), 3.26 (m, 1H), 2.96 (s, 3H), 2.88 (m, 1H), 2.68-2.84 (m, 2H), 2.29 (m, 1H), 2.18 (s, 3H), 1.91 (m, 1H), 1.45-1.71 (m, 5H), 0.61 (m, 1H), 0.38 (m, 2H), 0.05- 0.14 (m, 2H) |
| 132 | 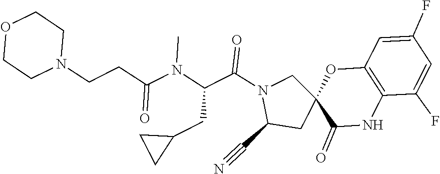 [SMILES: CN(C(=O)CCN1CCOCC1)[C@@H](CC1CC1)C(=O)N1C[C@@]2(C[C@H]1C#N)Oc1cc(F)cc(F)c1NC2=O] | 532 [M + H]⁺ | ¹H NMR (500 MHz, 365K, DMSO-d₆) δ 10.90 (br. s., 1H), 6.91 (m, 1H), 6.60 (m, 1H), 5.17 (m, 1H), 4.96 (m, 1H), 4.13 (m, 1H), 3.95 (m, 1H), 3.51 (t, 4H), 2.89 (s, 3H), 2.78 (m, 2H), 2.39- 2.50 (m, 3H), 2.38-2.25 (m, 5H), 1.65 (m, 1H), 1.51-1.61 (m, 1H), 0.60 (m, 1H), 0.33-0.42 (m, 2H), 0.02-0.12 (m, 2H) |
| 133 | 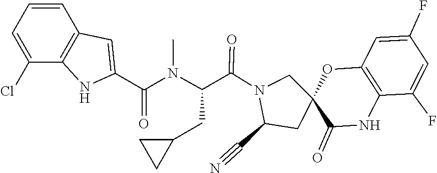 [SMILES: CN(C(=O)c1cc2cccc(Cl)c2[nH]1)[C@@H](CC1CC1)C(=O)N1C[C@@]2(C[C@H]1C#N)Oc1cc(F)cc(F)c1NC2=O] | 568 [M + H]⁺ | ¹H NMR (500 MHz, 363K, DMSO-d₆) δ 10.8-11.0 (br. s., 2H), 7.54 (d, 1H), 7.25 (dd, 1H), 7.04 (t, 1H), 6.85 (s, 1H), 6.74 (m, 1H), 6.66 (m, 1H), 5.29 (m, 1H), 5.02 (m, 1H), 4.00-4.25 (m, 2H), 3.20 (s, 3H), 2.80 (m, 2H), 1.80 (m, 2H), 0.74 (m, 1H), 0.44 (m, 2H), 0.15 (m, 2H) |
| 134 | 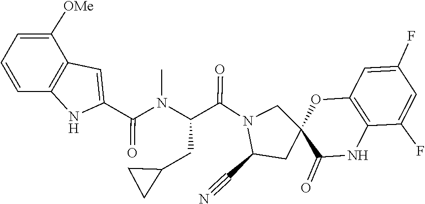 [SMILES: COc1cccc2[nH]c(C(=O)N(C)[C@@H](CC3CC3)C(=O)N3C[C@@]4(C[C@H]3C#N)Oc3cc(F)cc(F)c3NC4=O)cc12] | 564 [M + H]⁺ | ¹H NMR (500 MHz, 363K, DMSO-d₆) δ 11.12 (s, 1H), 11.00 (br. s., 1H), 7.11 (t, 1H), 7.03 (m, 1H), 6.86 (m, 1H), 6.73 (m, 1H), 6.67 (m, 1H), 6.51 (d, 1H), 5.35 (m, 1H), 5.04 (m, 1H), 4.22 (m, 1H), 4.06-4.16 (m, 1H), 3.89 (s, 3H), 3.27 (s, 3H), 2.71-2.86 (m, 2H), 1.80 (m, 2H), 0.73 (m, 1H), 0.44 (m, 2H), 0.17 (m, 2H) |
| 135 | 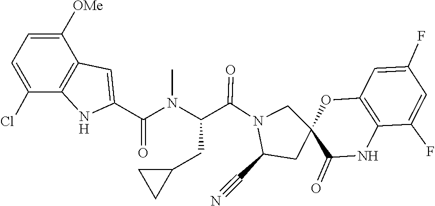 [SMILES: COc1ccc(Cl)c2[nH]c(C(=O)N(C)[C@@H](CC3CC3)C(=O)N3C[C@@]4(C[C@H]3C#N)Oc3cc(F)cc(F)c3NC4=O)cc12] | 598 [M + H]⁺ | ¹H NMR (500 MHz, 363K, DMSO-d₆) δ 11.0 (br. s., 2H), 7.18 (d, 1H), 6.87 (s, 1H), 6.75 (m, 1H), 6.67 (m, 1H), 6.57 (d, 1H), 5.31 (m, 1H), 5.05 (m, 1H), 4.03-4.28 (m, 2H), 3.90 (s, 3H), 3.23 (s, 3H), 2.81 (m, 2H), 1.82 (m, 2H), 0.76 (m, 1H), 0.46 (m, 2H), 0.17 (m, 2H) |
| 136 | 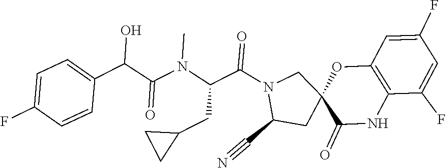 [SMILES: CN(C(=O)C(O)c1ccc(F)cc1)[C@@H](CC1CC1)C(=O)N1C[C@@]2(C[C@H]1C#N)Oc1cc(F)cc(F)c1NC2=O] | 543 [M + H]⁺ | ¹H NMR (500 MHz, 363K, DMSO-d₆) δ 11.0 (br. s., 1H), 7.31 (m, 2H), 7.07 (m, 1H), 6.95 (m, 2H), 6.52-6.76 (m, 1H), 5.33 (m, 1H), 5.07-5.28 (m, 2H), 4.85- 5.02 (m, 1H), 3.85-4.22 (m, 2H), 2.86 (s, 3H), 2.67-2.81 (m, 2H), 1.48-1.79 (m, 2H), 0.22-0.69 (m, 3H), −0.04-0.13 (m, 2H) |
| 137 | 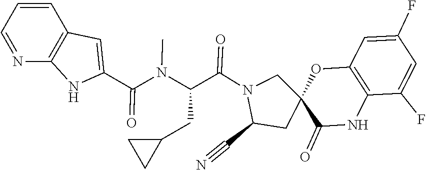 [SMILES: CN(C(=O)c1cc2cccnc2[nH]1)[C@@H](CC1CC1)C(=O)N1C[C@@]2(C[C@H]1C#N)Oc1cc(F)cc(F)c1NC2=O] | 535 [M + H]⁺ | ¹H NMR (500 MHz, 363K, DMSO-d₆) δ 11.56 (s, 1H), 10.95 (br. s., 1H), 8.31 (d, 1H), 7.97 (d, 1H), 7.08 (m, 1H), 6.72-6.80 (m, 2H), 6.67-6.71 (m, 1H), 5.27 (m, 1H), 5.02 (m, 1H), 4.01-4.25 (m, 2H), 3.21 (s, 3H), 2.81-2.76 (m, 2H), 1.79 (m, 2H), 0.74 (m, 1H), 0.43 (m, 2H), 0.14 (m, 2H) |
| 138 | 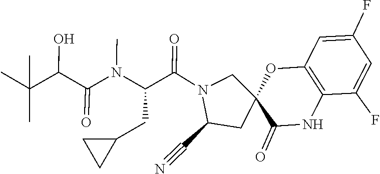 [SMILES: CN(C(=O)C(O)C(C)(C)C)[C@@H](CC1CC1)C(=O)N1C[C@@]2(C[C@H]1C#N)Oc1cc(F)cc(F)c1NC2=O] | 505 [M + H]⁺ | ¹H NMR (500 MHz, 363K, DMSO-d₆) δ 10.50 (br. s., 1H), 6.87-6.96 (m, 1H), 6.61-6.78 (m, 1H), 5.02-5.24 (m, 1H), 4.90- 5.02 (m, 1H), 3.98-4.41 (m, 4H), 3.00-3.12 (m, 3H), 2.67-2.83 (m, 2H), 1.55-1.76 (m, 2H), 0.82- 0.96 (m, 9H), 0.65 (m, 1H), 0.35- 0.47 (m, 2H), 0.08-0.18 (m, 2H) |
| 139 | 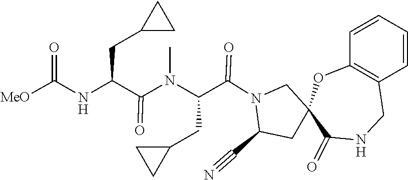 [SMILES: COC(=O)N[C@@H](CC1CC1)C(=O)N(C)[C@@H](CC1CC1)C(=O)N1C[C@@]2(C[C@H]1C#N)Oc1ccccc1CNC2=O] | 536 [M − H]⁻ | ¹H NMR (500 MHz, 363K, DMSO-d₆) δ 8.28 (m, 1H), 7.19- 7.29 (m, 2H), 7.06 (t, 1H), 7.00 (m, 1H), 6.79 (br. s., 1H), 5.20 (m, 1H), 4.92 (m, 1H), 4.40 (m, 1H), 4.22 (m, 1H), 3.98-4.18 (m, 2H), 3.89 (m, 1H), 3.42 (s, 3H), 2.92 (s, 3H), 2.64 (m, 1H), 2.52 (m, 1H), 1.48-1.64 (m, 2H), 1.26- 1.44 (m, 2H), 0.56 (m, 2H), 0.29 (m, 2H), 0.14 (m, 2H), 0.00 (m, 2H), −0.23 (m, 2H) |
| 140 | 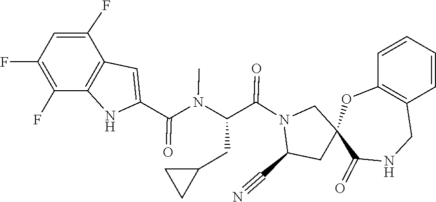 [SMILES: CN(C(=O)c1cc2c(F)cc(F)c(F)c2[nH]1)[C@@H](CC1CC1)C(=O)N1C[C@@]2(C[C@H]1C#N)Oc1ccccc1CNC2=O] | 564 [M − H]⁻ | ¹H NMR (500 MHz, 363K, DMSO-d₆) δ 12.20 (br. s., 1H), 8.37 (m, 1H), 7.27 (m, 1H), 7.01- 7.19 (m, 3H), 6.95 (m, 1H), 6.89 (s, 1H), 5.36 (m, 1H), 5.07 (m, 1H), 4.10-4.29 (m, 3H), 4.04 (m, 1H), 3.24 (s, 3H), 2.83 (m, 1H), 2.67 (m, 1H), 1.81 (m, 2H), 0.74 (m, 1H), 0.44 (m, 2H), 0.17 (m, 2H) |
| 141 | 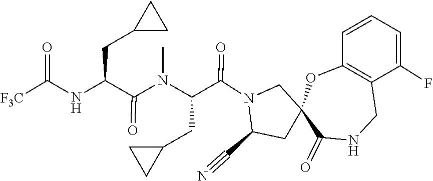 [SMILES: CN(C(=O)[C@H](CC1CC1)NC(=O)C(F)(F)F)[C@@H](CC1CC1)C(=O)N1C[C@@]2(C[C@H]1C#N)Oc1cccc(F)c1CNC2=O] | 592 [M − H]⁻ | ¹H NMR (500 MHz, 368K, DMSO-d₆) δ 9.20 (br. s., 1H), 8.41 (m, 1H), 7.33 (m, 1H), 7. 06 (t, 1H), 6.99 (m, 1H), 5.27 (m, 1H), 5.02 (m, 1H), 4.81 (m, 1H), 4.23-4.34 (m, 3H), 4.04 (m, 1H), 3.04 (s, 3H), 2.79 (m, 1H), 2.65 (m, 1H), 1.51-1.73 (m, 4H), 0.58- 0.74 (m, 2H), 0.22-0.44 (m, 4H), 0.10 (m, 2H), −0.13-0.00 (m, 2H) |
| 142 | 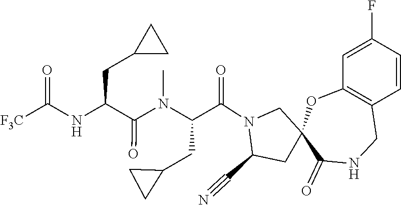 [SMILES: CN(C(=O)[C@H](CC1CC1)NC(=O)C(F)(F)F)[C@@H](CC1CC1)C(=O)N1C[C@@]2(C[C@H]1C#N)Oc1cc(F)ccc1CNC2=O] | 592 [M − H]⁻ | ¹H NMR (500 MHz, 364K, DMSO-d₆) δ ppm 9.20 (br. s., 1H), 8.43 (m, 1H), 7.38 (m, 1H), 7.09 (m, 1H), 6.99 (m, 1H), 5.27 (m, 1H), 5.08 (m, 1H), 4.81 (m, 1H), 4.22-4.35 (m, 2H), 3.99- 4.15 (m, 2H), 3.00-3.06 (m, 3H), 2.79 (m, 1H), 2.60-2.68 (m, 1H), 1.51-1.74 (m, 4H), 0.59-0.71 (m, 2H), 0.32-0.44 (m, 2H), 0.26 (m, 2H), 0.10 (m, 2H), −0.15-(−0.02) (m, 2H) |
| 143 | 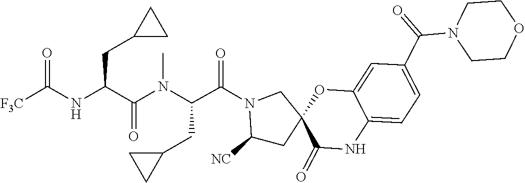 [SMILES: CN(C(=O)[C@H](CC1CC1)NC(=O)C(F)(F)F)[C@@H](CC1CC1)C(=O)N1C[C@@]2(C[C@H]1C#N)Oc1cc(C(=O)N3CCOCC3)ccc1NC2=O] | 675 [M + H]⁺ | ¹H NMR (500 MHz, 363K, DMSO-d₆) δ ppm 11.00 (br. s., 1H), 9.20 (br. s., 1H), 7.07 (m, 1H), 6.99 (m, 1H), 6.94 (s, 1H), 5.21 (m, 1H), 4.97 (m, 1H), 4.80 (m, 1H), 4.04-4.25 (m, 2H), 3.59 (m, 4H), 3.48 (m, 4H), 3.03 (s, 3H), 2.76 (m, 1H), 2.61 (m, 1H), 1.48-1.75 (m, 4H), 0.64 (m, 2H), 0.23-0.48 (m, 4H), 0.09 (m, 2H), 0.03 (m, 1H), −0.05 (m, 1H) |
| 144 | 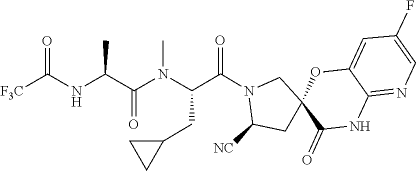 [SMILES: C[C@H](NC(=O)C(F)(F)F)C(=O)N(C)[C@@H](CC1CC1)C(=O)N1C[C@@]2(C[C@H]1C#N)Oc1cc(F)cnc1NC2=O] | 539 [M − H]⁻ | ¹H NMR (500 MHz, 364K, DMSO-d₆) δ ppm 11.45 (br. s., 1H), 9.20 (br. s., 1H), 7.96 (m, 1H), 7.38 (m, 1H), 5.13 (m, 1H), 4.97 (m, 1H), 4.72 (m, 1H), 3.98- 4.16 (m, 2H), 2.96 (m, 3H), 2.71- 2.85 (m, 2H), 1.65 (m, 2H), 1.18 (m, 3H), 0.61 (m, 1H), 0.32-0.44 (m, 2H), 0.07 (m, 2H) |
| 145 | 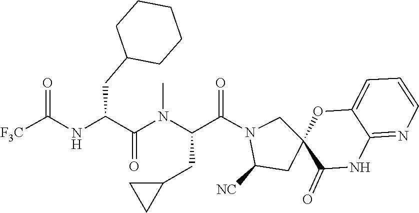 [SMILES: CN(C(=O)[C@@H](CC1CCCCC1)NC(=O)C(F)(F)F)[C@@H](CC1CC1)C(=O)N1C[C@@]2(C[C@H]1C#N)Oc1cccnc1NC2=O] | 605 [M + H]⁺ | ¹H NMR (500 MHz, 363K, DMSO-d₆) δ ppm 11.20 (br. s., 1H), 9.20 (br. s., 1H), 7.93 (m, 1H), 7.24 (m, 1H), 6.95 (m, 1H), 5.12 (m, 1H), 4.97 (m, 1H), 4.76 (m, 1H), 4.04-4.14 (m, 2H), 3.02 (s, 3H), 2.75 (m, 1H), 2.66 (m, 1H), 1.55-1.80 (m, 8H), 1.50 (m, 1H), 1.25 (m, 1H), 1.14 (m, 3H), 0.81-1.01 (m, 2H), 0.62 (m, 1H), 0.32-0.47 (m, 2H), 0.11 (m, 2H) |
| 146 | 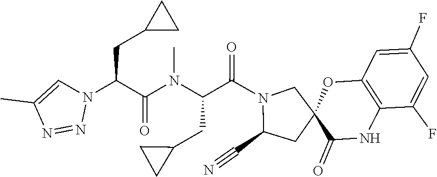 [SMILES: Cc1cn([C@@H](CC2CC2)C(=O)N(C)[C@@H](CC2CC2)C(=O)N2C[C@@]3(C[C@H]2C#N)Oc2cc(F)cc(F)c2NC3=O)nn1] | 568 [M + H]⁺ | ¹H NMR (500 MHz, 363K, DMSO-d₆) δ ppm 10.30 (br. s., 1H), 7.72 (s, 1H), 6.95 (t, 1H), 6.73 (d, 1H), 5.73 (m, 1H), 5.11 (m, 1H), 4.96 (m, 1H), 4.21 (m, 1H), 4.06 (m, 1H), 3.05 (s, 3H), 2.67-2.86 (m, 2H), 2.20 (s, 3H), 1.77-1.97 (m, 2H), 1.61 (m, 2H), 0.50 (m, 2H), 0.30-0.37 (m, 4H), 0.01-0.09 (m, 3H), −0.11-(-0.04) (m, 1H) |
| 147 | 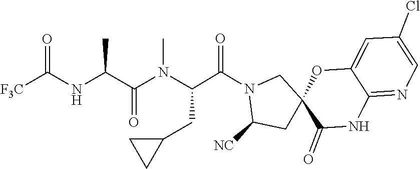 [SMILES: C[C@H](NC(=O)C(F)(F)F)C(=O)N(C)[C@@H](CC1CC1)C(=O)N1C[C@@]2(C[C@H]1C#N)Oc1cc(Cl)cnc1NC2=O] | 555 [M − H]⁻ | ¹H NMR (500 MHz, 363K, DMSO-d₆) δ ppm 11.55 (br. s., 1H), 9.20 (br. s., 1H), 8.00 (s, 1H), 7.49 (s, 1H), 5.14 (m, 1H), 4.98 (m, 1H), 4.73 (m, 1H), 3.98- 4.19 (m, 2H), 2.97 (s, 3H), 2.67- 2.97 (m, 2H), 1.65 (m, 2H), 1.20 (m, 3H), 0.62 (m, 1H), 0.30-0.45 (m, 2H), 0.09 (m, 2H) |
| 148 | 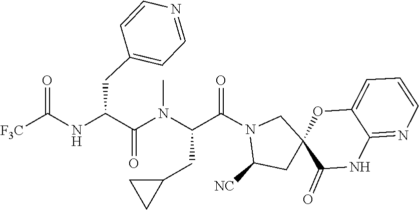 | 600 [M + H]⁺ | ¹H NMR (500 MHz, 363K, DMSO-d₆) δ ppm 11.30 (br. s., 1H), 9.45 (br. s., 1H), 8.41 (d, 2H), 7.95 (m, 1H), 7.23 (m, 3H), 6.95 (m, 1H), 4.91-5.18 (m, 3H), 4.11 (m, 2H), 3.05 (m, 2H), 3.01 (s, 3H), 2.58-2.81 (m, 2H), 1.64 (m, 1H), 1.56 (m, 1H), 0.55 (m, 1H), 0.37 (m, 2H), 0.10 (m, 2H) |
| 149 | 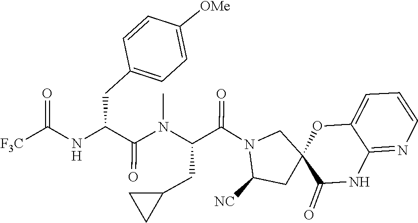 [SMILES: COc1ccc(C[C@@H](NC(=O)C(F)(F)F)C(=O)N(C)[C@@H](CC2CC2)C(=O)N2C[C@@]3(C[C@H]2C#N)Oc2cccnc2NC3=O)cc1] | 629 [M + H]⁺ | ¹H NMR (500 MHz, 363K, DMSO-d₆) δ ppm 9.20 (br. s., 2H), 7.93 (m, 1H), 7.21 (m, 1H), 7.13 (d, 2H), 6.95 (m, 1H), 6.79 (d, 2H), 5.07 (m, 1H), 4.96 (m, 1H), 4.91 (m, 1H), 4.04 -. 4.16 (m, 2H), 3.70 (s, 3H), 2.96 (s, 3H), 2.86-2.94 (m, 2H), 2.58-2.80 (m, 2H), 1.61 (m, 1H), 1.48 (m, 1H), 0.51 (m, 1H), 0.32-0.41 (m, 2H), 0.09 (m, 2H) |
| 150 | 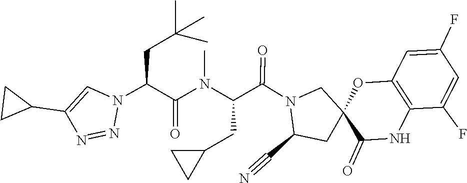 [SMILES: CN(C(=O)[C@H](CC(C)(C)C)n1cc(C2CC2)nn1)[C@@H](CC1CC1)C(=O)N1C[C@@]2(C[C@H]1C#N)Oc1cc(F)cc(F)c1NC2=O] | 610 [M + H]⁺ | ¹H NMR (500 MHz, 363K, DMSO-d₆) δ ppm 10.15 (br. s., 1H), 7.73 (s, 1H), 6.95 (m, 1H), 6.75 (m, 1H), 5.71 (m, 1H), 5.10 (m, 1H), 4.96 (m, 1H), 4.02-4.20 (m, 2H), 3.07 (s, 3H), 2.63-2.85 (m, 2H), 1.95 (m, 1H), 1.86-1.92 (m, 2H), 1.60 (m, 2H), 0.86 (m, 2H), 0.72 (s, 9H), 0.62-0.69 (m, 2H), 0.41 (m, 1H), 0.25 (m, 2H), −0.04-0.06 (m, 2H) |
| 151 | 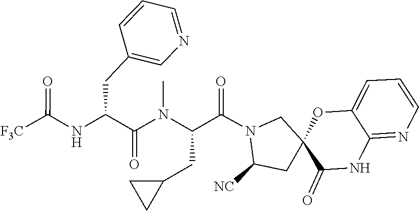 | 600 [M + H]⁺ | ¹H NMR (500 MHz, 363K, DMSO-d₆) δ ppm 8.9-10.9 (br. s., 2H), 8.43 (m, 1H), 8.38 (m, 1H), 7.93 (m, 1H), 7.63 (m, 1H), 7.20- 7.27 (m, 2H), 6.95 (m, 1H), 5.09 (m, 1H), 4.93-5.04 (m, 2H), 4.11 (m, 2H), 3.04-3.11 (m, 1H), 3.01 (s, 3H), 2.95-3.00 (m, 1H) 2.58- 2.79 (m, 2H), 1.63 (m, 1H), 1.54 (m, 1H), 0.54 (m, 1H), 0.33-0.42 (m, 2H), 0.06-0.14 (m, 2H) |
| 152 | 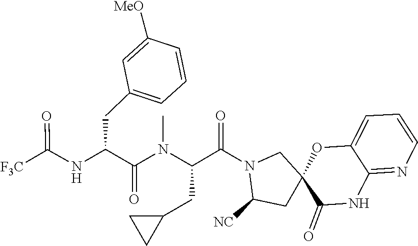 [SMILES: COc1cccc(C[C@@H](NC(=O)C(F)(F)F)C(=O)N(C)[C@@H](CC2CC2)C(=O)N2C[C@@]3(C[C@H]2C#N)Oc2cccnc2NC3=O)c1] | 629 [M + H]⁺ | ¹H NMR (500 MHz, 363K, DMSO-d₆) δ ppm 11.20 (br. s., 1H), 9.30 (br. s., 1H), 7.93 (m, 1H), 7.22 (m, 1H), 7.14 (m, 1H), 6.95-6.93 (m, 1H), 6.79 (m, 1H), 6.76-6.75 (m, 2H), 5.08 (m, 1H), 4.96 (m, 2H), 4.04 -. 4.17 (m, 2H), 3.71 (s, 3H), 2.90-3.05 (m, 5H), 2.58-2.80 (m, 2H), 1.62 (m, 1H), 1.49 (m, 1H), 0.52 (m, 1H), 0.38 (m, 2H), 0.08 (m, 2H) |
| 153 | 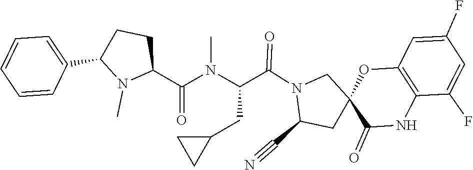 [SMILES: CN(C(=O)[C@@H]1CC[C@@H](c2ccccc2)N1C)[C@@H](CC1CC1)C(=O)N1C[C@@]2(C[C@H]1C#N)Oc1cc(F)cc(F)c1NC2=O] | 578 [M + H]⁺ | ¹H NMR (500 MHz, 363K, DMSO-d₆) δ ppm 10.95 (br.s., 1H), 7.22-7.34 (m, 4H), 7.18 (m, 1H), 6.94 (m, 1H), 6.74 (m, 1H), 5.22 (m, 1H), 4.97 (m, 1H), 4.27 (m, 1H), 3.98-4.23 (m, 3H), 2.97 (s, 3H), 2.80 (m, 2H), 2.01-2.25 (m, 5H), 1.64 (m, 2H), 1.53 (m, 2H), 0.64 (m, 1H), 0.31-0.48 (m, 2H), 0.05-0.19 (m, 2H) |
| 154 | 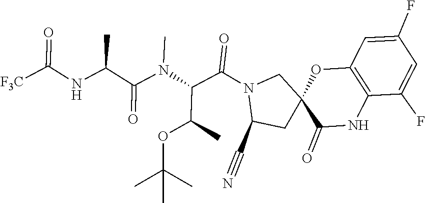 [SMILES: C[C@H](NC(=O)C(F)(F)F)C(=O)N(C)[C@H](C(=O)N1C[C@@]2(C[C@H]1C#N)Oc1cc(F)cc(F)c1NC2=O)[C@@H](C)OC(C)(C)C] | 602 [M − H]⁻ | ¹H NMR (500 MHz, 363K, DMSO-d₆) δ ppm 11.00 (br. s., 1H), 9.20 (br. s., 1H), 6.92 (t, 1H), 6.71 (m, 1H), 4.90-5.16 (m, 2H), 4.82 (m, 1H), 3.98-4.21 (m, 3H), 3.11 (s, 3H), 2.64-2.87 (m, 2H), 1.17-1.36 (m, 3H), 1.03- 1.16 (m, 12H) |
| 155 | 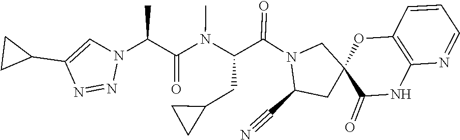 [SMILES: C[C@@H](C(=O)N(C)[C@@H](CC1CC1)C(=O)N1C[C@@]2(C[C@H]1C#N)Oc1cccnc1NC2=O)n1cc(C2CC2)nn1] | 519 [M + H]⁺ | ¹H NMR (500 MHz, 363K, DMSO-d₆) δ ppm 10.15 (br. s., 1H), 7.97 (m, 1H), 7.68 (s, 1H), 7.333 (m, 1H), 7.01 (m, 1H), 5.73 (m, 1H), 4.91-5.17 (m, 2H), 3.93- 4.18 (m, 2H), 2.97 (s, 3H), 2.79 (m, 2H), 1.90 (m, 1H), 1.61 (m, 2H), 1.47 (m, 3H), 0.86 (m, 2H), 0.67 (m, 2H), 0.48 (m, 1H), 0.30 (m, 2H), 0.03 (m, 2H) |
| 156 | 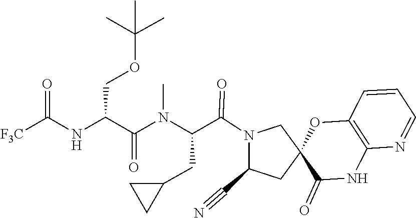 [SMILES: CN(C(=O)[C@@H](COC(C)(C)C)NC(=O)C(F)(F)F)[C@@H](CC1CC1)C(=O)N1C[C@@]2(C[C@H]1C#N)Oc1cccnc1NC2=O] | 595 [M + H]⁺ | ¹H NMR (500 MHz, 363K, DMSO-d₆) δ 11.30 (br. s., 1H), 9.00 (br. s., 1H), 7.93 (m, 1H), 7.24 (m, 1H), 6.94 (m, 1H), 5.16 (m, 1H), 4.97 (m, 1H), 4.83 (m, 1H), 4.03-4.14 (m, 2H), 3.50- 3.62 (m, 2H), 3.04 (s, 3H), 2.75 (m, 1H), 2.63 (m, 1H), 1.66 (m, 2H), 1.09 (s, 9H), 0.68 (m, 1H), 0.34-0.45 (m, 2H), 0.11 (m, 2H) |
| 157 | 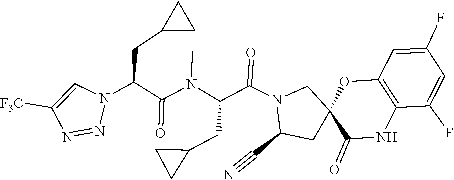 [SMILES: CN(C(=O)[C@H](CC1CC1)n1cc(C(F)(F)F)nn1)[C@@H](CC1CC1)C(=O)N1C[C@@]2(C[C@H]1C#N)Oc1cc(F)cc(F)c1NC2=O] | 620 [M − H]⁻ | ¹H NMR (500 MHz, 363K, DMSO-d₆) δ 11.00 (br. s., 1H), 8.80 (s, 1H), 6.92 (t, 1H), 6.72 (d, 1H), 5.98 (m, 1H), 5.12 (m, 1H), 4.96 (m, 1H), 4.03-4.23 (m, 2H), 3.12 (s, 3H), 2.67-2.85 (m, 2H), 1.88-2.06 (m, 2H), 1.65 (m, 2H), 0.45-0.63 (m, 2H), 0.25-0.38 (m, 4H), 0.01-0.010 (m, 3H), −0.07 (m, 1H) |
| 158 | 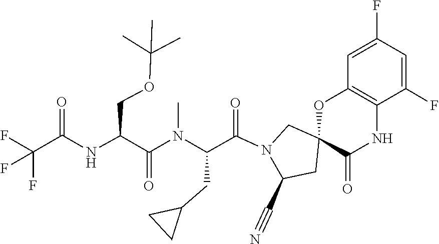 [SMILES: CN(C(=O)[C@H](COC(C)(C)C)NC(=O)C(F)(F)F)[C@@H](CC1CC1)C(=O)N1C[C@@]2(C[C@H]1C#N)Oc1cc(F)cc(F)c1NC2=O] | 652 [M + Na]⁺ | ¹H NMR (400 MHz, 80° C., DMSO-d₆) δ 8.91-9.79 (m, 1H), 6.81-7.11 (m, 1H), 6.51-6.75 (m, 1H), 5.01-5.22 (m, 1H), 4.79- 5.00 (m, 1H), 4.61-4.78 (m, 1H), 3.92-4.21 (m, 2H), 3.38-3.56 (m, 2H), 2.86-2.97 (m, 3H), 2.51- 2.74 (m, 2H), 1.49-1.73 (m, 2H), 0.76-1.30 (m, 9H), 0.48-0.70 (m, 1H), 0.19-0.47 (m, 2H), −0.10 - 0.18 (m, 2H) |
| 159 | 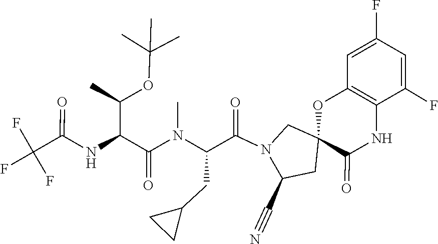 [SMILES: C[C@@H](OC(C)(C)C)[C@H](NC(=O)C(F)(F)F)C(=O)N(C)[C@@H](CC1CC1)C(=O)N1C[C@@]2(C[C@H]1C#N)Oc1cc(F)cc(F)c1NC2=O] | 666 [M + Na]⁺ | ¹H NMR (400 MHz, 80° C., DMSO-d₆) δ 8.82 (br, 1H), 6.80- 6.95 (m, 1H), 6.49-6.68 (m, 1H), 5.01-5.21 (m, 1H), 4.73-4.81 (m, 1H), 4.41-4.69 (m, 1H), 4.09- 4.22 (m, 1H), 3.98-4.08 (m, 1H), 3.82-3.97 (m, 1H), 3.02-3.39 (m, 3H), 2.53-2.73 (m, 2H), 1.46- 1.73 (m, 2H), 0.98-1.11 (m, 9H), 0.71-0.97 (m, 3H), 0.43-0.61 (m, 1H), 0.18-0.28 (m, 2H), −0.10 - 0.10 (m, 2H) |
| 160 | 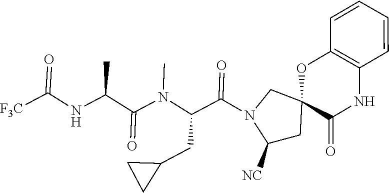 [SMILES: C[C@H](NC(=O)C(F)(F)F)C(=O)N(C)[C@@H](CC1CC1)C(=O)N1C[C@@]2(C[C@H]1C#N)Oc1ccccc1NC2=O] | 544 [M + Na]⁺ | ¹H NMR (400 MHz, 80° C., DMSO-d₆) δ 11.11 (br, 1H), 9.25 (br, 1H), 6.81-7.09 (m, 4H), 5.12- 5.22 (m, 1H), 4.91-5.05 (m, 1H), 4.55-4.71 (m, 1H), 3.85-4.09 (m, 2H), 2.87 (s, 3H), 2.61-2.90 (m, 2H), 1.51-1.65 (m, 2H), 0.90- 1.12 (m, 3H), 0.53-0.71(m, 1H), 0.21-0.42(m, 2H), 0.01-0.15 (m, 2H) |
| 161 | 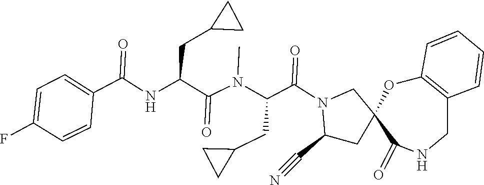 [SMILES: CN(C(=O)[C@H](CC1CC1)NC(=O)c1ccc(F)cc1)[C@@H](CC1CC1)C(=O)N1C[C@@]2(C[C@H]1C#N)Oc1ccccc1CNC2=O] | 624 [M + Na]⁺ | ¹H NMR (400 MHz, 80º C, DMSO-d₆) δ 8.20-8.55 (m, 2H), 7.65-7.96 (m, 2H), 7.20-7.40 (m, 2H), 6.82-7.19 (m, 4H), 5.10- 5.35 (m, 1H), 4.91-5.09 (m, 1H), 4.75-4.90 (m, 1H), 4.10-4.40 (m, 2H), 3.80-4.09 (m, 2H), 2.95- 3.00 (m, 3H), 2.48-2.75 (m, 2H), 1.35-1.80 (m, 4H), 0.48-0.80 (m, 2H), 0.20-0.40 (m, 2H), 0.05- 0.19 (m, 2H), −0.05-0.04 (m, 2H), −0.35 − −0.12 (m, 2H). |
| 162 | 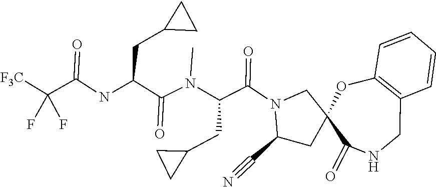 | 648 [M + Na]⁺ | ¹H NMR (400 MHz, 80º C, DMSO-d₆) δ 9.36 (br, 1H), 8.40 (s, 1H), 7.20-7.55 (m, 2H), 6.85- 7.19 (m, 2H), 4.60-5.50 (m, 3H), 3.80-4.40 (m, 4H), 2.78-2.95 (m, 3H), 2.45-2.77 (m, 2H), 1.30- 1.80 (m, 4H), 0.40-0.80 (m, 2H), 0.12-0.39 (m, 4H), −0.05-0.11 (m, 2H), −0.40 − −0.06 (m, 2H). |
| 163 | 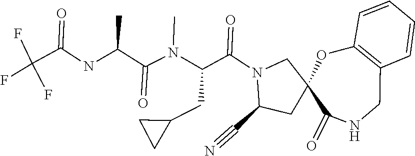 | 558 [M + Na]⁺ | ¹H NMR (400 MHz, DMSO-d₆) δ 9.28 (br, 1H), 8.28-8.69 (m, 1H), 6.91-7.59 (m, 4H), 4.53-5.37 (m, 3H), 3.81-4.39 (m, 4H), 2.81- 2.99 (m, 3H), 2.48-2.89 (m, 2H), 1.09-1.83 (m, 5H), 0.49-0.77 (m, 1H), 0.19-0.48 (m, 2H), −0.30- 0.18 (m, 2H) |
| 164 | 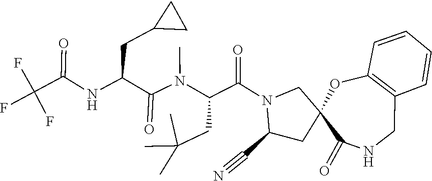 [SMILES: CN(C(=O)[C@H](CC1CC1)NC(=O)C(F)(F)F)[C@@H](CC(C)(C)C)C(=O)N1C[C@@]2(C[C@H]1C#N)Oc1ccccc1CNC2=O] | 614 [M + Na]⁺ | ¹H NMR (400 MHz, 60° C., DMSO-d₆) δ 9.18-9.63 (m, 1H), 8.29-8.61 (m, 1H), 7.11-7.36 (m, 2H), 6.86-7.10 (m, 2H), 5.08- 5.39 (m, 1H), 4.81-5.02 (m, 1H), 4.51-4.73 (m, 1H), 4.19-4.39 (m, 1H), 4.03-4.18 (m, 1H), 3.90- 4.02 (m, 1H), 3.71-3.89 (m, 1H), 2.71-2.96 (m, 3H), 2.41-2.70 (m, 2H), 1.63-1.99 (m, 1H), 1.21- 1.60 (m, 3H), 0.63-0.81 (m, 9H), 0.32-0.62 (m, 1H), −0.12-0.31 (m, |
| | | | 2H), −0.58 − −0.18 (m, 2H). |
| 165 | 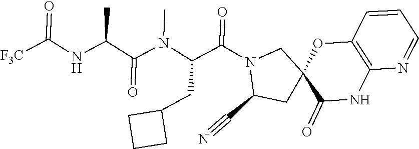 [SMILES: C[C@H](NC(=O)C(F)(F)F)C(=O)N(C)[C@@H](CC1CCC1)C(=O)N1C[C@@]2(C[C@H]1C#N)Oc1cccnc1NC2=O] | 559 [M + Na]⁺ | ¹H NMR (400 MHz, 80° C., DMSO-d₆) δ 11.43 (br, 1H), 9.36 (br, 1H), 7.80-8.20 (m, 1H), 7.25- 7.50 (m, 1H), 6.81-7.24 (m, 1H), 4.85-5.30 (m, 2H), 4.50-4.84 (m, 1H), 3.80-4.30 (m, 2H), 2.90- 3.10 (m, 3H), 2.63-2.89 (m, 2H), 2.10-2.30 (m, 1H), 1.50-2.09 (m, 8H), 1.10-1.40 (m, 3H). |
| 166 | 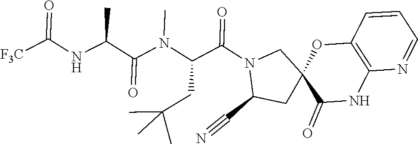 [SMILES: C[C@H](NC(=O)C(F)(F)F)C(=O)N(C)[C@@H](CC(C)(C)C)C(=O)N1C[C@@]2(C[C@H]1C#N)Oc1cccnc1NC2=O] | 561 [M + Na]⁺ | ¹H NMR (400 MHz, 80° C., DMSO-d₆) δ 11.50 (br, 0.45H), 9.35 (br, 1H), 7.90-8.10 (m, 1H), 7.20-7.45 (m, 1H), 6.95-7.10 (m, 1H), 5.18-5.35 (m, 1H), 4.85- 5.10 (m, 1H), 4.60-4.80 (m, 1H), 4.00-4.30 (m, 2H), 2.98 (s, 3H), 2.65-2.85 (m, 2H), 1.90-2.10 (m, 1H), 1.40-1.53 (m, 1H), 1.05- 1.25 (m, 3H), 0.90 (s, 9H). |
| 167 | 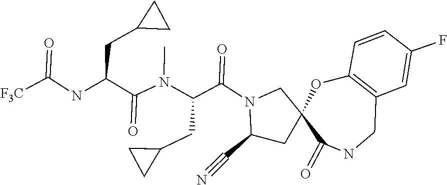 | 616 [M + Na]⁺ | ¹H NMR (400 MHz, 80º C, DMSO-d₆) δ 9.41 (br, 1H), 8.55 (br, 1H), 7.00-7.50 (m, 3H), 5.30 (br, 1H), 5.08 (br, 1H), 4.84 (br, 1H), 4.00-4.50 (m, 4H), 3.05 (br, 3H), 2.70-2.90 (m, 1H), 2.60- 2.69 (m, 1H), 1.50-1.90 (m, 4H), 0.60-0.80 (m, 2H), 0.20-0.59 (m, 4H), 0.10-0.19(m, 2H), −0.80 − 0.09 (m, 2H). |
| 168 | 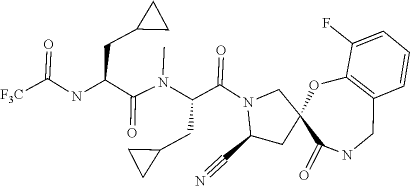 | 616 [M + Na]⁺ | ¹H NMR (400 MHz, 80° C., DMSO-d₆) δ 9.37 (br, 1H), 8.56 (br, 1H), 7.21-7.30 (m, 3H), 5.30- 5.41 (m, 1H), 4.72-4.99 (m, 2H), 4.41-4.49 (m, 1H), 4.20-4.27 (m, 1H), 3.96-4.18 (m, 2H), 3.04 (s, 3H), 2.65-2.80 (m, 2H), 1.64- 1.79 (m, 3H), 1.52-1.60 (m, 1H), 0.55-0.75 (m, 2H), 0.32-0.44 (m, 2H), 0.18-0.30 (m, 2H), 0.05- 0.16 (m, 2H), −0.21 − −0.04 (m, 2H). |
| 169 | 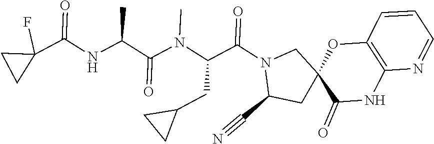 [SMILES: C[C@H](NC(=O)C1(F)CC1)C(=O)N(C)[C@@H](CC1CC1)C(=O)N1C[C@@]2(C[C@H]1C#N)Oc1cccnc1NC2=O] | 535 [M + Na]⁺ | ¹H NMR (400 MHz, 80° C., DMSO-d₆) δ 11.42 (br, 1H), 7.81- 8.22 (m, 2H), 7.22-7.57 (m, 1H), 6.86-7.21 (m, 1H), 4.90-5.35 (m, 2H), 4.55-4.89 (m, 1H), 3.85- 4.25 (m, 2H), 2.95-3.05 (m, 3H), 2.70-2.89 (m, 2H), 1.50-1.80 (m, 2H), 1.00-1.34 (m, 7H), 0.51- 0.76 (m, 1H), 0.28-0.50 (m, 2H), 0.00-0.20 (m, 2H). |
| 170 | 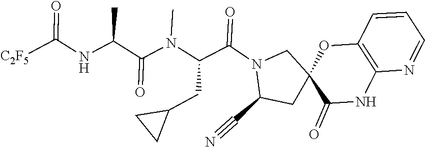 | 595 [M + Na]⁺ | ¹H NMR (400 MHz, 80° C., DMSO-d₆) δ 11.40 (br, 1H), 9.42 (br, 1H), 7.90-8.10 (m, 1H), 7.20- 7.50 (m, 1H), 6.90-7.12 (m, 1H), 5.10-5.35 (m, 1H), 4.89-5.09 (m, 1H), 4.60-4.88 (m, 1H), 3.90- 4.25 (m, 2H), 2.91-3.05 (m, 3H), 2.60-2.90 (m, 2H), 1.55-1.70 (m, 2H), 1.10-1.35 (m, 3H), 0.52- 0.80 (m, 1H), 0.26-0.51 (m, 2H), |
| | | | 0.00-0.25 (m, 2H) |
| 171 | 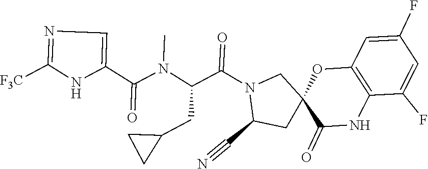 [SMILES: CN(C(=O)c1cnc(C(F)(F)F)[nH]1)[C@@H](CC1CC1)C(=O)N1C[C@@]2(C[C@H]1C#N)Oc1cc(F)cc(F)c1NC2=O] | 575 [M + Na]⁺ | ¹H NMR (400 MHz, 80° C., DMSO-d₆) δ 11.05 (br, 1H), 7.65 (s, 1H), 6.75-6.90 (m, 1H), 6.51- 6.70 (m, 1H), 5.12-5.85 (m, 1H), 5.00 (br, 1H), 4.21- 4.35 (m, 1H), 3.91-4.12 (m,1H), 3.21-3.28 (m, 3H), 2.78-2.82 (m, 2H), 1.61- 1.90 (m, 2H), 0.51-0.75 (m, 1H), 0.21-0.48 (m, 2H), 0.03-0.20 (m, 2H). |
| 172 | 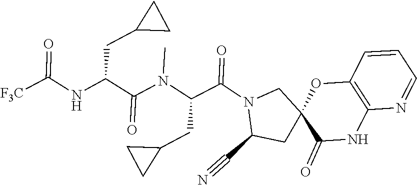 [SMILES: CN(C(=O)[C@@H](CC1CC1)NC(=O)C(F)(F)F)[C@@H](CC1CC1)C(=O)N1C[C@@]2(C[C@H]1C#N)Oc1cccnc1NC2=O] | 585 [M + Na]⁺ | ¹H NMR (400 MHz, 80° C., DMSO-d₆) δ 11.22 (br, 1H), 9.10 (br, 1H), 7.70-8.05 (m, 1H), 6.98- 7.25 (m, 1H), 6.70-6.92 (m, 1H), 4.80-5.12 (m, 2H), 4.52-4.79 (m, 1H), 3.85-4.15 (m, 2H), 2.85- 2.93 (m, 3H), 2.60-2.70 (m, 1H), 2.42-2.59 (m, 1H), 1.40-1.65 (m, 4H), 0.40-0.65 (m, 2H), 0.20- 0.36 (m, 4H), −0.10-0.10 (m, 4H). |
| 173 | 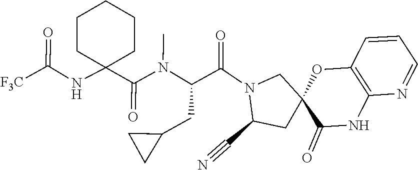 [SMILES: CN(C(=O)C1(NC(=O)C(F)(F)F)CCCCC1)[C@@H](CC1CC1)C(=O)N1C[C@@]2(C[C@H]1C#N)Oc1cccnc1NC2=O] | 599 [M + Na]⁺ | ¹H NMR (400 MHz, 80° C., DMSO-d₆) δ 9.09 (br, 1H), 7.81 - 7.99 (m, 1H), 7.16-7.32 (m, 1H), 6.86-7.04 (m, 1H), 4.93-5.07 (m, 1H), 4.81-4.92 (m, 1H), 4.02- 4.26 (m, 1H), 3.87-4.01 (m, 1H), 2.91 (s, 3H), 2.58-2.73 (m, 2H), 1.86-1.95 (m, 2H), 1.74-1.85 (m, 1H), 1.52-1.65 (m, 2H), 1.33- 1.51 (m, 5H), 1.22-1.32 (m, 1H), 1.10-1.21 (m, 1H), 0.46-0.60 (m, |
| | | | 1H), 0.24-0.37 (m, 2H), −0.01- |
| | | | 0.09 (m, 2H). |
| 174 | 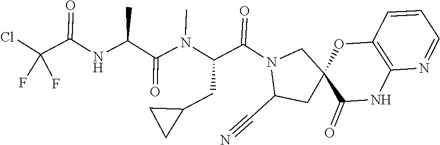 [SMILES: C[C@H](NC(=O)C(F)(F)Cl)C(=O)N(C)[C@@H](CC1CC1)C(=O)N1C[C@@]2(CC1C#N)Oc1cccnc1NC2=O] | 561 [M + Na]⁺ | ¹H NMR (400 MHz, 80º C, DMSO-d₆) δ 11.40 (br, 1H), 9.04 (br, 1H), 7.80-7.95 (m, 1H), 7.18- 7.35 (m, 1H), 6.82-6.96 (m, 1H), 4.97-5.20 (m, 1H), 4.78-4.96 (m, 1H), 4.48-4.70 (m, 1H), 3.80- 4.10 (m, 2H), 2.88 (s, 3H), 2.50- 2.80 (m, 2H), 1.45-1.65 (m, 2H), 0.95-1.20 (m, 3H), 0.45-0.62 (m, |
| | | | 1H), 0.20-0.40 (m, 2H), −0.08- |
| | | | 0.10 (m, 2H). |
| 175 | 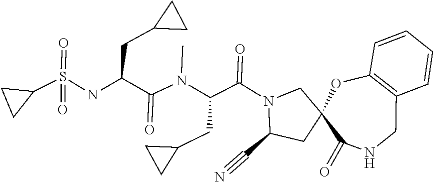 | 341 (fragment peak). | ¹H NMR (400 MHz, 80° C., DMSO-d₆) δ 8.26-8.47 (m, 1H), 7.16-7.29 (m, 2H), 6.96-7.13 (m, 2H), 6.81-6.98 (m, 1H), 5.09- 5.29 (m, 1H), 4.78-4.99 (m, 1H), 4.03-4.29 (m, 3H), 3.72-4.02 (m, 2H), 2.88-2.91 (m, 3H), 2.53- 2.67 (m, 1H), 2.41-2.52 (m, 1H), 2.21-2.31 (m, 1H), 1.45-1.61 (m, 2H), 1.32-1.49 (m, 1H), 1.18- 1.31 (m, 1H), 0.69-0.89 (m, 4H), 0.59-0.68 (m, 1H), 0.45-0.57 (m, |
| | | | 1H), 0.18-0.38 (m, 2H), 0.02- |
| | | | 0.19 (m, 2H), −0.08-0.06 (m, |
| | | | 2H), −0.38 − −0.17 (m, 2H). |
| 176 | 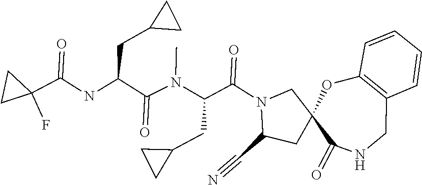 | 588 [M + Na]⁺ | ¹H NMR (400 MHz, 80° C., DMSO-d₆) δ 8.37 (br, 1H), 7.72- 7.86 (m, 1H), 7.17-7.41 (m, 2H), 6.90-7.16 (m, 2H), 5.20 (br, 1H), 4.87-5.04 (m, 1H), 4.61-4.82 (m, 1H), 4.09-4.31 (m, 2H), 3.94- 4.08 (m, 1H), 3.79-3.93 (m, 1H), 2.92-2.96 (m, 3H), 2.58-2.83 (m, 1H), 2.48-2.57 (m, 1H), 1.31- 1.66 (m, 4H), 1.13-1.29 (m, 2H), 0.96-1.12 (m, 2H), 0.48-0.66 (m, 2H), 0.21-0.34 (m, 2H), 0.08- |
| | | | 0.19 (m, 2H), −0.08-0.07 (m, |
| | | | 2H), −0.28 − −0.17 (m, 2H). |
| 177 | 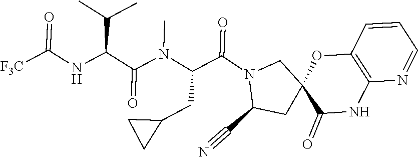 [SMILES: CC(C)[C@H](NC(=O)C(F)(F)F)C(=O)N(C)[C@@H](CC1CC1)C(=O)N1C[C@@]2(C[C@H]1C#N)Oc1cccnc1NC2=O] | 573 [M + Na]⁺ | ¹H NMR (400 MHz, 80° C., DMSO-d₆) δ 11.36 (br, 1H), 9.17 (br, 1H), 7.79-8.00 (m, 1H), 7.12- 7.30 (m, 1H), 6.81-7.01 (m, 1H), 5.01-5.22 (m, 1H), 4.78-4.99 (m, 1H), 4.36-4.50 (m, 1H), 3.99- 4.19 (m, 2H), 2.95-2.99 (m, 3H), 2.68-2.79 (m, 1H), 2.53-2.67 (m, 1H), 1.93-2.12 (m, 1H), 1.50- 1.67 (m, 2H), 0.65-0.85 (m, 6H), |
| | | | 0.40-0.64 (m, 1H), 0.19-0.38 (m, |
| | | | 2H), −0.05-0.11 (m, 2H). |
| 178 | 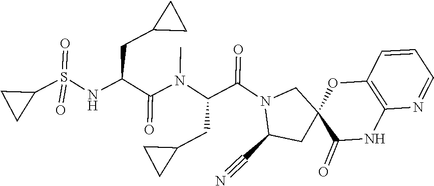 [SMILES: CN(C(=O)[C@H](CC1CC1)NS(=O)(=O)C1CC1)[C@@H](CC1CC1)C(=O)N1C[C@@]2(C[C@H]1C#N)Oc1cccnc1NC2=O] | 593 [M + Na]⁺ | ¹H NMR (400 MHz, 80° C., DMSO-d₆) δ 11.41 (br, 1H), 7.96- 8.03 (m, 1H), 7.29 (d, J = 8.0 Hz, 1H), 6.94-7.09 (m, 2H), 5.13- 5.18 (m, 1H), 4.89-5.03 (m, 1H), 4.27-4.37 (m, 1H), 4.07-4.26 (m, 2H), 2.94-3.04 (m, 3H), 2.75- 2.86 (m, 1H), 2.60-2.74 (m, 1H), 2.36-2.47 (m, 1H), 1.62-1.74 (m, 2H), 1.46-1.60 (m, 1H), 1.33- 1.45 (m, 1H), 0.82-0.96 (m, 4H), 0.61-0.81 (m, 2H), 0.30-0.51 (m, |
| | | | 4H), 0.08-0.20 (m, 2H), −0.02- |
| | | | 0.07 (m, 2H). |
| 179 | 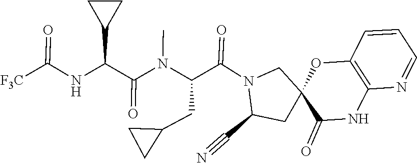 [SMILES: CN(C(=O)[C@@H](NC(=O)C(F)(F)F)C1CC1)[C@@H](CC1CC1)C(=O)N1C[C@@]2(C[C@H]1C#N)Oc1cccnc1NC2=O] | 571 [M + Na]⁺ | ¹H NMR (400 MHz, 80° C. DMSO-d₆) δ 11.40 (br, 1H), 9.48 (br, 1H), 7.90-8.11 (m, 1H), 7.28- 7.51 (m, 1H), 6.90-7.18 (m, 1H), 5.19 (br, 1H), 5.00 (br, 1H), 4.22- 4.50 (m, 1H), 3.98-4.20 (m, 2H), 3.01-3.05 (m, 3H), 2.80-2.90 (m, 1H), 2.60-2.75 (m, 1H), 1.55- 1.80 (m, 2H), 1.10-1.30 (m, 1H), 0.53-0.73 (m, 1H), 0.20-0.50 (m, 6H), 0.10-0.18 (m, 2H). |
| 180 | 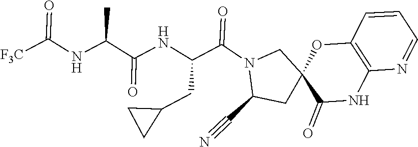 [SMILES: C[C@H](NC(=O)C(F)(F)F)C(=O)N[C@@H](CC1CC1)C(=O)N1C[C@@]2(C[C@H]1C#N)Oc1cccnc1NC2=O] | 531 [M + Na]⁺ | ¹H NMR (400 MHz, 80º C, DMSO-d₆) δ 11.75 (s, 0.432H), 9.45 (s, 0.518H), 8.15 (br, 1H), 7.99 (br, 1H), 7.36-7.38 (m, 1H), 6.99-7.02 (m, 1H), 4.99 (br, 1H), 4.40-4.70 (m, 2H), 4.15- 4.35 (m, 1H), 4.02-4.05 (m, 1H), 2.81 (br, 2H), 1.60 (br, 2H), 1.20-1.45 (m, 3H), 0.76 (br, 1H), 0.30-0.50 (m, 2H), 0.05-0.25 |
| | | | (m, 2H). |
| 181 | 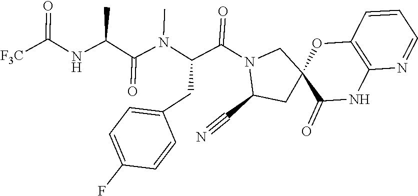 [SMILES: C[C@H](NC(=O)C(F)(F)F)C(=O)N(C)[C@@H](Cc1ccc(F)cc1)C(=O)N1C[C@@]2(C[C@H]1C#N)Oc1cccnc1NC2=O] | 599 [M + Na]⁺ | ¹H NMR (400 MHz, 80° C., DMSO-d₆) δ 11.38 (br, 1H), 9.26 (br, 1H), 7.95 (s, 1H), 7.16-7.40 (m, 3H), 6.91-7.10 (m, 3H), 5.21- 5.40 (m, 1H), 4.90-5.00 (m, 1H), 4.65-4.73 (m, 1H), 3.86-4.10 (m, 1H), 3.70-3.80 (m, 1H), 3.19- 3.22 (m, 1H), 3.01-3.10 (m, 3H), 2.91-2.96 (m, 1H), 2.72-2.83 (m, 1H), 2.60-2.70 (m, 1H), 0.95- 1.24 (m, 3H). |
| 182 | 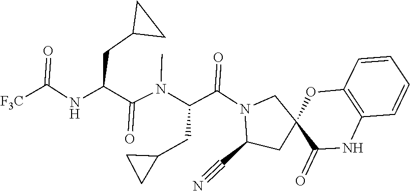 [SMILES: CN(C(=O)[C@H](CC1CC1)NC(=O)C(F)(F)F)[C@@H](CC1CC1)C(=O)N1C[C@@]2(C[C@H]1C#N)Oc1ccccc1NC2=O] | 562 [M + H]⁺ | ¹H NMR (400 MHz, 80° C., DMSO-d₆) δ 10.85 (s, 1H), 9.35 (br, 1H), 6.90-7.10 (m, 3H), 6.80- 6.89 (m, 1H), 5.22 (br, 1H), 4.93 (br, 1H), 4.75 (br, 1H), 4.00- 4.20 (m, 2H), 3.00 (s, 3H), 2.70- 2.80 (m, 1H), 2.55-2.60 (m, 1H), 1.40-1.80 (m, 4H), 0.50-0.70 (m, 2H), 0.20-0.45 (m, 4H), 0.00- 0.15 (m, 3H), −0.10 − −0.01 (m, 1H). |
| 183 | 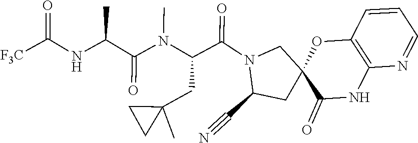 [SMILES: C[C@H](NC(=O)C(F)(F)F)C(=O)N(C)[C@@H](CC1(C)CC1)C(=O)N1C[C@@]2(C[C@H]1C#N)Oc1cccnc1NC2=O] | 559 [M + Na]⁺ | ¹H NMR (400 MHz, 80° C., DMSO-d₆) δ 11.30 (br, 1H), 9.38 (br, 1H), 7.92-8.11 (m, 1H), 7.17- 7.51 (m, 1H), 6.92-7.13 (m, 1H), 5.11-5.47 (m, 1H), 4.88-5.09 (m, 1H), 4.57-4.86 (m, 1H), 3.91- 4.22 (m, 2H), 2.91-3.01 (m, 3H), 2.79-2.87 (m, 1H), 2.64-2.78 (m, 1H), 1.88-2.01 (m, 1H), 1.47- |
| | | | 1.61 (m, 1H), 1.07-1.21 (m, 3H), |
| | | | 0.98-1.05 (m, 3H), 0.24-0.39 (m, |
| | | | 2H), 0.15-0.27 (m, 2H). |
| 184 | 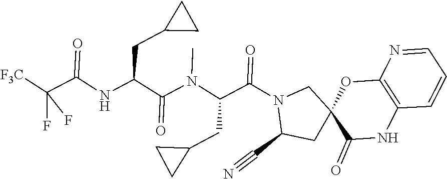 [SMILES: CN(C(=O)[C@H](CC1CC1)NC(=O)C(F)(F)C(F)(F)F)[C@@H](CC1CC1)C(=O)N1C[C@@]2(C[C@H]1C#N)Oc1ncccc1NC2=O] | 613 [M + H]⁺ | ¹H NMR (400 MHz, 80° C., DMSO-d₆) δ 9.35 (br, 1H), 7.70- 7.88 (m, 1H), 7.20-7.32 (m, 1H), 6.90-7.10 (m, 1H), 5.20 (br, 1H), 4.87 (br, 1H), 4.65-4.78 (m, 1H), 3.92-4.16 (m, 2H), 2.90-2.96 (m, 3H), 2.66-2.80 (m, 1H), 2.52- 2.64 (m, 1H), 1.61-1.71 (m, 2H), 1.52-1.60 (m, 1H), 1.40-1.51 (m, 1H), 0.54-0.72 (m, 2H), 0.24- 0.47 (m, 4H), −0.00-0.17 (m, 3H), −0.10 − −0.01 (m, 1H). |
| 185 | 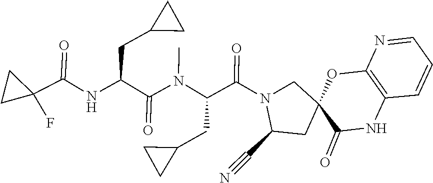 [SMILES: CN(C(=O)[C@H](CC1CC1)NC(=O)C1(F)CC1)[C@@H](CC1CC1)C(=O)N1C[C@@]2(C[C@H]1C#N)Oc1ncccc1NC2=O] | 553 [M + H]⁺ | ¹H NMR (400 MHz, 80° C., DMSO-d₆) δ 7.70-8.00 (m, 2H), 7.28-7.41 (m, 1H), 7.00-7.20 (m, 1H), 5.27 (br, 1H), 4.97 (br, 1H), 4.70-4.88 (m, 1H), 4.06-4.21 (m, 2H), 3.02-3.08 (m, 3H), 2.77- 2.90 (m, 1H), 2.61-2.76 (m, 1H), 1.60-1.80 (m, 2H), 1.40-1.58 (m, 2H), 1.10-1.38 (m, 4H), 0.65 (br, 2H), 0.25-0.50 (m, 4H), 0.05- 0.20 (m, 2H), −0.10-0.04 (m, 2H). |
| 186 | 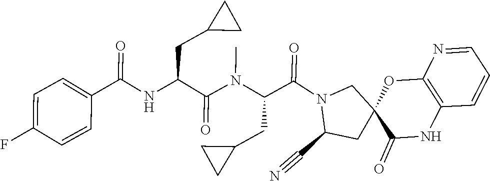 [SMILES: CN(C(=O)[C@H](CC1CC1)NC(=O)c1ccc(F)cc1)[C@@H](CC1CC1)C(=O)N1C[C@@]2(C[C@H]1C#N)Oc1ncccc1NC2=O] | 571 [M + H]⁺ | ¹H NMR (400 MHz, 80° C., DMSO-d₆) δ 7.62-7.72 (m, 2H), 7.51-7.59 (m, 1H), 7.01-7.11 (m, 3H), 6.73-6.89 (m, 1H), 6.73- 6.89 (m, 1H), 4.71-4.92 (m, 1H), 3.83-3.99 (m, 2H), 2.58-2.72 (m, 5H), 2.08-2.11 (m, 2H), 1.38- 1.51 (m, 2H), 0.71-0.89 (m, 1H), 0.32-0.59 (m, 1H), 0.08-0.29 (m, 4H), −0.02-0.06 (m, 2H), −0.17 − −0.01 (m, 2H). |
| 187 | 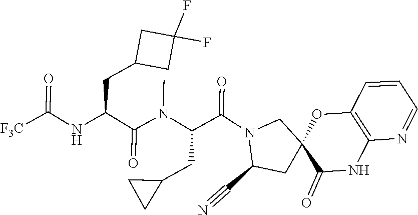 [SMILES: CN(C(=O)[C@H](CC1CC(F)(F)C1)NC(=O)C(F)(F)F)[C@@H](CC1CC1)C(=O)N1C[C@@]2(C[C@H]1C#N)Oc1cccnc1NC2=O] | 635 [M + Na]⁺ | ¹H NMR (400 MHz, 80° C., DMSO-d₆) δ 11.40 (br, 1H), 9.39 (br, 1H), 7.94-8.01 (m, 1H), 7.27- 7.40 (m, 1H), 6.95-7.05 (m, 1H), 5.05-5.29 (m, 1H), 4.85-5.04 (m, 1H), 4.60-4.74 (m, 1H), 4.00- 4.24 (m, 2H), 3.00 (s, 3H), 2.73- 2.86 (m, 1H), 2.60-2.72 (m, 1H), 2.48-2.59 (m, 2H), 2.00-2.29 (m, 3H), 1.71-1.93 (m, 2H), 1.55- 1.70 (m, 2H), 0.52-0.68 (m, 1H), 0.25-0.47 (m, 2H), 0.01-0.17 (m, 2H). |
| 188 | 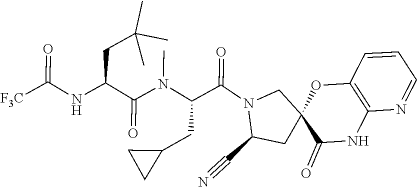 [SMILES: CN(C(=O)[C@H](CC(C)(C)C)NC(=O)C(F)(F)F)[C@@H](CC1CC1)C(=O)N1C[C@@]2(C[C@H]1C#N)Oc1cccnc1NC2=O] | 601 [M + Na]⁺ | ¹H NMR (400 MHz, 80° C., DMSO-d₆) δ 11.39 (br, 1H), 9.46 (m, 1H), 7.96-8.04 (m, 1H), 7.33- 7.43 (m, 1H), 6.98-7.09 (m, 1H), 5.12-5.28 (m, 1H), 4.92-5.06 (m, 1H), 4.75-4.87 (m, 1H), 4.02- 4.22 (m, 2H), 3.00 (s, 3H), 2.74- 2.85 (m, 1H), 2.56-2.70 (m, 1H), 1.70-1.81 (m, 1H), 1.62-1.69 (m, 2H), 1.46-1.57 (m, 1H), 0.84 (s, 9H), 0.56-0.68 (m, 1H), 0.31- 0.44 (m, 2H), 0.04-0.17 (m, 2H). |
| 189 | 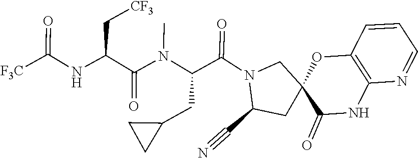 [SMILES: CN(C(=O)[C@H](CC(F)(F)F)NC(=O)C(F)(F)F)[C@@H](CC1CC1)C(=O)N1C[C@@]2(C[C@H]1C#N)Oc1cccnc1NC2=O] | 613 [M + Na]⁺ | ¹H NMR (400 MHz, 80° C., DMSO-d₆) δ 9.60-11.70 (br, 1H), 7.80-8.00 (m, 1H), 7.20-7.40 (m, 1H), 6.80-7.00 (m, 1H), 4.80- 5.15 (m, 3H), 3.85-4.20 (m, 2H), 2.95 (s, 3H), 2.50-2.80 (m, 4H), 1.50-1.70 (m, 2H), 0.50-0.60 (m, 1H), 0.20-0.40 (m, 2H), 0.00- 0.10 (m, 2H). |
| 190 | 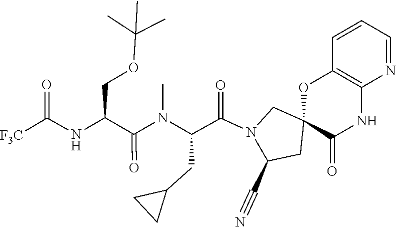 [SMILES: CN(C(=O)[C@H](COC(C)(C)C)NC(=O)C(F)(F)F)[C@@H](CC1CC1)C(=O)N1C[C@@]2(C[C@H]1C#N)Oc1cccnc1NC2=O] | 617 [M + Na]⁺ | ¹H NMR (400 MHz, 80° C., DMSO-d₆) δ 8.70-10.00 (m, 1H), 7.73-8.06 (m, 1H), 7.11-7.37 (m, 1H), 6.71-7.09 (m, 1H), 5.01- 5.21 (m, 1H), 4.78-4.99 (m, 1H), 4.65-4.76 (m, 1H), 3.86-4.19 (m, 2H), 3.38-3.47 (m, 2H), 2.91- 2.95 (m, 3H), 2.62-2.73 (m, 1H), 2.52-2.61 (m, 1H), 1.52-1.72 (m, 2H), 0.99 (s, 9H), 0.43-0.62 (m, 1H), 0.21-0.39 (m, 2H), −0.11- 0.12 (m, 2H). |
| 191 | 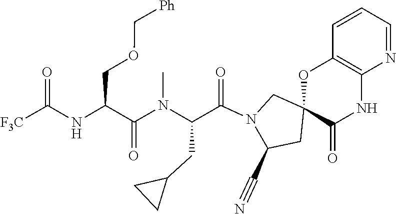 [SMILES: CN(C(=O)[C@H](COCc1ccccc1)NC(=O)C(F)(F)F)[C@@H](CC1CC1)C(=O)N1C[C@@]2(C[C@H]1C#N)Oc1cccnc1NC2=O] | 651 [M + Na]⁺ | ¹H NMR (400 MHz, DMSO-d₆) δ 11.5 (br, 1H), 9.65 (br, 1H), 7.80-8.00 (m, 1H), 7.10- 7.30 (m, 6H), 6.80-7.00 (m, 1H), 5.00-5.20 (m, 1H), 4.80- 5.00 (m, 2H), 4.25-4.45 (m, 2H), 4.05 (br, 2H), 3.45-3.65 (m, 2H), 2.95 (s, 3H), 2.60-2.80 (m, 2H), 1.50-1.70 (m, 2H), 0.50-0.70 (m, 1H), 0.20-0.40 (m, 2H), −0.10- 0.10 (m, 2H). |
| 192 | 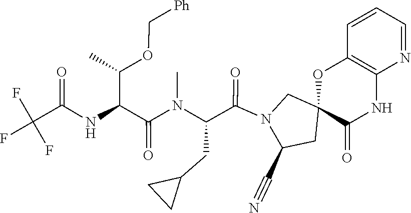 [SMILES: C[C@H](OCc1ccccc1)[C@H](NC(=O)C(F)(F)F)C(=O)N(C)[C@@H](CC1CC1)C(=O)N1C[C@@]2(C[C@H]1C#N)Oc1cccnc1NC2=O] | 665 [M + Na]⁺ | ¹H NMR (400 MHz, DMSO-d₆) δ 11.00-11.70 (br, 1H), 9.10-9.70 (br, 1H), 7.80-8.00 (m, 1H), 7.20- 7.30 (m, 2H), 7.10-7.19 (m, 3H), 6.85-7.05 (m, 1H), 6.75-6.84 (m, 1H), 5.00-5.20 (m, 1H), 4.70- 4.90 (m, 2H), 4.30-4.50 (m, 2H), 4.00-4.20 (m, 2H), 3.80-4.00 (m, 1H), 3.05 (s, 3H), 2.50-2.70 (m, 2H), 1.50-1.70 (m, 2H), 0.90- 1.10 (m, 3H), 0.50-0.60 (m, 1H), 0.20-0.40 (m, 2H), 0.00-0.10 (m, 2H). |
| 193 | 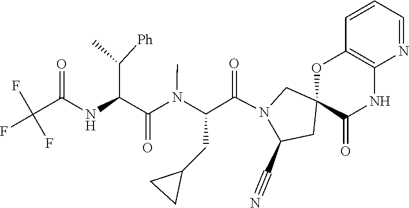 [SMILES: C[C@H](c1ccccc1)[C@H](NC(=O)C(F)(F)F)C(=O)N(C)[C@@H](CC1CC1)C(=O)N1C[C@@]2(C[C@H]1C#N)Oc1cccnc1NC2=O] | 635 [M + Na]⁺ | ¹H NMR (400 MHz, 80° C., DMSO-d₆) δ 11.42 (br, 1H), 9.56 (br, 1H), 7.90-8.05 (m, 1H), 7.25- 7.42 (m, 1H), 7.12-7.24 (m, 4H), 7.04-7.11 (m, 1H), 6.90-7.03 (m, 1H), 4.98-5.10 (m, 1H), 4.84- 4.97 (m, 1H), 4.75-4.83 (m, 1H), 4.00-4.20 (m, 1H), 3.80-3.98 (m, 1H), 3.20-3.40 (m, 1H), 2.68- 2.80 (m, 4H), 2.50-2.65 (m, 1H), 1.40-1.60 (m, 2H), 1.05-1.25 (m, 3H), 0.41-0.60 (m, 1H), 0.20- 0.40 (m, 2H), 0.10-0.08 (m, 2H). |
| 194 | 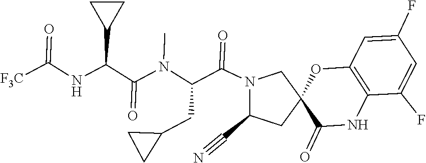 [SMILES: CN(C(=O)[C@@H](NC(=O)C(F)(F)F)C1CC1)[C@@H](CC1CC1)C(=O)N1C[C@@]2(C[C@H]1C#N)Oc1cc(F)cc(F)c1NC2=O] | 606 [M + Na]⁺ | ¹H NMR (400 MHz, 80° C. DMSO-d₆) δ 9.41 (br, 1H), 6.81- 6.95 (m, 1H), 6.58-6.71 (m, 1H), 5.01-5.21 (m, 1H), 4.81-4.98 (m, 1H), 4.21-4.31 (m, 1H), 3.95- 4.18 (m, 2H), 2.90-3.91 (m, 3H), 2.73-2.81 (m, 1H), 2.53-2.65 (m, 1H), 1.41-1.72 (m, 2H), 1.01- 1.51 (m, 1H), 0.45-0.65 (m, 1H), 0.32-0.40 (m, 2H), 0.19-0.30 (m, 4H), 0.01-0.10 (m, 2H). |
| 195 | 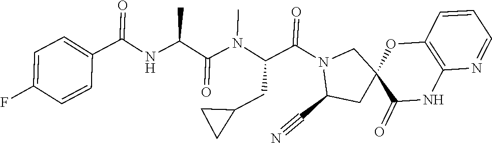 [SMILES: C[C@H](NC(=O)c1ccc(F)cc1)C(=O)N(C)[C@@H](CC1CC1)C(=O)N1C[C@@]2(C[C@H]1C#N)Oc1cccnc1NC2=O] | 571 [M + Na]⁺ | ¹H NMR (400 MHz, 80° C., DMSO-d₆) δ 11.43 (br, 1H), 8.34 (s, 1H), 7.75-8.15 (m, 3H), 7.15- 7.55 (m, 3H), 6.88-7.14 (m, 1H), 4.60-5.50 (m, 3H), 3.88-4.30 (m, 2H), 2.93-3.10 (m, 3H), 2.65- 2.92 (m, 2H), 1.50-1.90 (m, 2H), 1.00-1.46 (m, 3H), 0.56-0.75 (m, 1H), 0.25-0.55 (m, 2H), 0.00- |
| | | | 0.24 (m, 2H). |
| 196 | 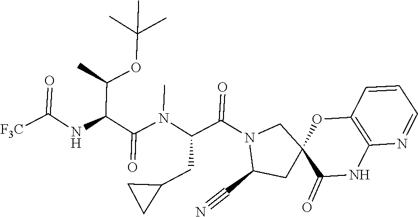 [SMILES: C[C@@H](OC(C)(C)C)[C@H](NC(=O)C(F)(F)F)C(=O)N(C)[C@@H](CC1CC1)C(=O)N1C[C@@]2(C[C@H]1C#N)Oc1cccnc1NC2=O] | 631 [M + Na]⁺ | ¹H NMR (400 MHz, 80° C., DMSO-d₆) δ 8.93 (br, 1H), 7.87- 8.11 (m, 1H), 7.28-7.41 (m, 1H), 6.91-7.12 (m, 1H), 5.11-5.32 (m, 1H), 4.84-5.09 (m, 1H), 4.61- 4.76 (m, 1H), 4.11-4.32 (m, 2H), 3.87-4.04 (m, 1H), 3.09 (s, 3H), 2.63-2.82 (m, 2H), 1.61-1.81 (m, 2H), 1.11-1.18 (m, 9H), 0.91- 0.99 (m, 3H), 0.58-0.71 (m, 1H), 0.29-0.49 (m, 2H), 0.07-0.18 (m, 2H). |
| 197 | 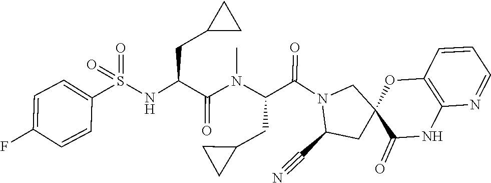 [SMILES: CN(C(=O)[C@H](CC1CC1)NS(=O)(=O)c1ccc(F)cc1)[C@@H](CC1CC1)C(=O)N1C[C@@]2(C[C@H]1C#N)Oc1cccnc1NC2=O] | 647 [M + Na]⁺ | ¹H NMR (400 MHz, 80° C., DMSO-d₆) δ 7.90-8.10 (m, 1H), 7.70-7.89 (m, 2H), 7.20-7.40 (m, 3H), 6.95-7.10 (m, 1H), 4.80- 5.00 (m, 2H), 4.20-4.35 (m, 1H), 4.00-4.10 (m, 2H), 2.95 (s, 3H), 2.70-2.80 (m, 1H), 2.50-2.69 (m, 1H), 1.55-1.70 (m, 1H), 1.30- 1.45 (m, 1H), 1.20-1.29 (m, 2H), 0.50-0.65 (m, 1H), 0.40-0.49 (m, 1H), 0.35-0.39 (m, 2H), 0.20- 0.30 (m, 1H), 0.15-0.19 (m, 1H), |
| | | | 0.00-0.10 (m, 2H), −0.01 − −0.10 |
| | | | (m, 2H). |
| 198 | 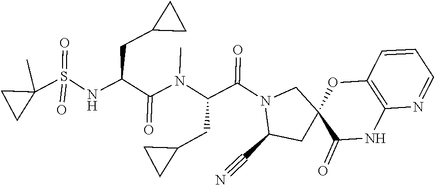 [SMILES: CN(C(=O)[C@H](CC1CC1)NS(=O)(=O)C1(C)CC1)[C@@H](CC1CC1)C(=O)N1C[C@@]2(C[C@H]1C#N)Oc1cccnc1NC2=O] | 607 [M + Na]⁺ | ¹H NMR (400 MHz, 80° C. DMSO-d₆) δ 11.45 (br, 1H), 7.90- 8.15 (m, 1H), 7.21-7.41 (m, 1H), 6.91-7.15 (m, 2H), 5.15-5.32 (m, 1H), 4.81-5.10 (m, 1H), 4.00- 4.51 (m, 3H), 3.00-3.05 (m, 3H), 2.80-2.85 (m, 1H), 2.55-2.62 (m, 1H), 1.60-1.70 (m, 2H), 1.45- 1.58 (m, 1H), 1.28-1.40 (m, 4H), 1.00-1.20 (m, 2H), 0.61-0.80 (m, 4H), 0.30-0.50 (m, 4H), 0.05- 0.15 (m, 2H), 0.01-0.03 (m, 2H) |
| 199 | 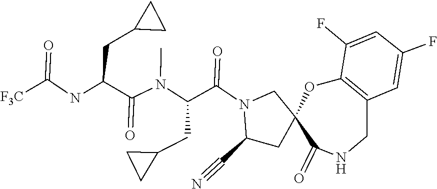 | 634 [M + Na]⁺ | ¹H NMR (400 MHz, 80° C., DMSO-d₆) δ 9.10-9.60 (m, 1H), 8.40-8.80 (m, 1H), 7.20-7.30 (m, 1H), 7.10-7.18 (m, 1H), 5.25- 5.45 (m, 1H), 4.70-4.90 (m, 2H), 4.31-4.40 (m, 1H), 4.20-4.30 (m, 1H), 4.10-4.18 (m, 1H), 4.00- 4.09 (m, 1H), 3.00-3.05 (m, 3H), 2.60-2.85 (m, 2H), 1.60-1.80 (m, 3H) 1.40-1.58 m, 1H), 0.55-0.80 (m, 2H), 0.20-0.50 (m, 4H), 0.01- 0.18 (m, 2H), −0.02 − −0.01 (m, |
| | | | 2H). |
| 200 | 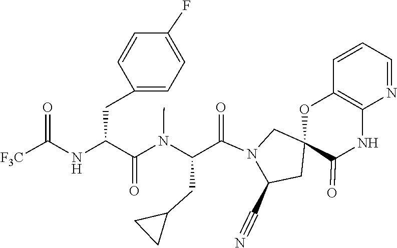 [SMILES: CN(C(=O)[C@@H](Cc1ccc(F)cc1)NC(=O)C(F)(F)F)[C@@H](CC1CC1)C(=O)N1C[C@@]2(C[C@H]1C#N)Oc1cccnc1NC2=O] | 639 [M + Na]⁺ | ¹H NMR (400 MHz, 80° C., DMSO-d₆) δ 11.27 (br, 1H), 9.37 (br, 1H), 7.80-7.90 (m, 1H), 7.05- 7.25 (m, 3H), 7.02-7.09 (m, 2H), 6.89-7.01 (m, 1H), 4.95-5.16 (m, 1H), 4.70-4.94 (m, 2H), 3.88- 4.20 (m, 2H), 2.89-2.95 (m, 4H), 2.80-2.88 (m, 1H), 2.60-2.75 (m, 1H), 2.50-2.68 (m, 1H), 1.49- 1.65 (m, 1H), 1.30-1.48 (m, 1H), 0.39-0.55 (m, 1H), 0.20-0.38 (m, 2H), −0.05-0.12 (m, 2H). |
| 201 | 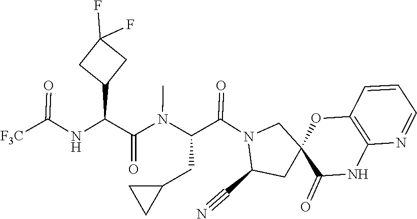 [SMILES: CN(C(=O)[C@@H](NC(=O)C(F)(F)F)C1CC(F)(F)C1)[C@@H](CC1CC1)C(=O)N1C[C@@]2(C[C@H]1C#N)Oc1cccnc1NC2=O] | 621 [M + Na]⁺ | ¹H NMR (400 MHz, 80° C., DMSO-d₆) δ 11.5 (br, 1H), 9.80 (br, 1H), 7.90-8.10 (m, 1H), 7.28- 7.60 (m, 1H), 6.90-7.25 (m, 1H), 5.15- 5.21 (m, 1H), 4.90-5.10 (m, 1H), 4.75-4.85 (m, 1H), 4.00- 4.20 (m, 2H), 2.90-3.00 (m, 3H), 2.80-2.88 (m, 1H), 2.65-2.78 (m, 1H), 2.55-2.63 (m, 1H), 2.40- 2.53 (m, 1H), 2.30-2.38 (m, 2H), 2.10-2.28 (m, 1H), 1.55-1.80 (m, 2H), 0.51-0.70 (m, 1H), 0.30- 0.42 (m, 2H), 0.01-0.20 (m, 2H). |
| 202 | 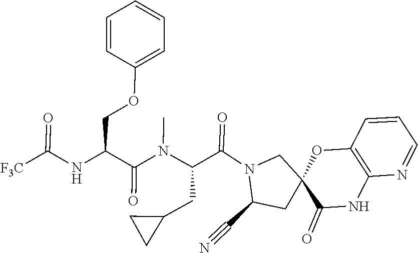 [SMILES: CN(C(=O)[C@H](COc1ccccc1)NC(=O)C(F)(F)F)[C@@H](CC1CC1)C(=O)N1C[C@@]2(C[C@H]1C#N)Oc1cccnc1NC2=O] | 637 [M + Na]⁺ | ¹H NMR (400 MHz, 80° C., DMSO-d₆) δ 11.5 (br, 1H), 9.70 (br, 1H), 7.70-8.00 (m, 1H), 7.00- 7.25 (m, 3H), 6.80-6.90 (m, 2H), 6.60-6.78 (m, 2H), 5.10-5.20 (m, 1H), 5.00-5.08 (m, 1H), 4.80- 4.98 (m, 1H), 3.90-4.20 (m, 4H), 2.90-3.00(m, 3H), 2.50-2.80 (m, 2H), 1.35-1.70 (m, 2H), 0.40- 0.60 (m, 1H), 0.20-0.38 (m, 2H), −0.01-0.15(m, 2H). |
| 203 | 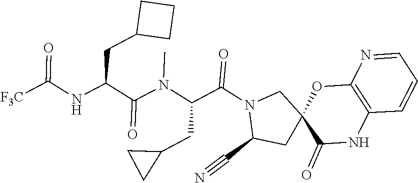 [SMILES: CN(C(=O)[C@H](CC1CCC1)NC(=O)C(F)(F)F)[C@@H](CC1CC1)C(=O)N1C[C@@]2(C[C@H]1C#N)Oc1ncccc1NC2=O] | 577 [M + H]⁺ | ¹H NMR (400 MHz, 80° C., DMSO-d₆) δ 9.35 (br, 1H), 7.78- 8.00 (m, 1H), 7.30-7.45 (m, 1H), 7.00-7.20 (m, 1H), 5.16-5.45 (m, 1H), 4.90-5.15 (m, 1H), 4.50- 4.70 (m, 1H), 4.05-4.30 (m, 2H), 2.98-3.01 (m, 3H), 2.80-2.89 (m, 1H), 2.64-2.76 (m, 1H), 2.20- 2.32 (m, 1H), 1.80-2.00 (m, 2H), 1.60-1.79 (m, 6H), 1.45-1.59 (m, 2H), 0.52-0.70 (m, 1H), 0.30- 0.48 (m, 2H), 0.05-0.17 (m, 2H). |
| 204 | 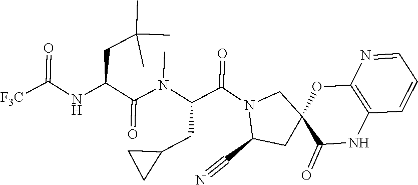 [SMILES: CN(C(=O)[C@H](CC(C)(C)C)NC(=O)C(F)(F)F)[C@@H](CC1CC1)C(=O)N1C[C@@]2(C[C@H]1C#N)Oc1ncccc1NC2=O] | 579 [M + H]⁺ | ¹H NMR (400 MHz, 80° C., DMSO-d₆) δ 10.98 (br, 1H), 9.38 (br, 1H), 7.70-7.80 (m, 1H), 7.18- 7.32 (m, 1H), 6.90-7.10 (m, 1H), 5.05-5.30 (m, 1H), 4.80-5.00 (m, 1H), 4.60-4.79 (m, 1H), 4.08- 4.22 (m, 1H), 3.90-4.07 (m, 1H), 2.85-2.90 (m, 3H), 2.62-2.80 (m, 1H), 2.48-2.61 (m, 1H), 1.50- 1.75 (m, 3H), 1.30-1.49 (m, 1H), 0.60-0.85 (m, 9H), 0.40-0.60 (m, 1H), 0.15-0.38 (m, 2H), −0.08- |
| | | | 0.09 (m, 2H). |
| 205 | 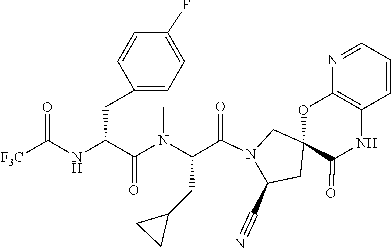 [SMILES: CN(C(=O)[C@@H](Cc1ccc(F)cc1)NC(=O)C(F)(F)F)[C@@H](CC1CC1)C(=O)N1C[C@@]2(C[C@H]1C#N)Oc1ncccc1NC2=O] | 617 [M + H]⁺ | ¹H NMR (400 MHz, 80° C., DMSO-d₆) δ 9.32 (br, 1H), 7.60- 7.80 (m, 1H), 7.10-7.30 (m, 3H), 6.80-7.08 (m, 3H), 4.95-5.16 (m, 1H), 4.72-4.94 (m, 2H), 3.90- 4.20 (m, 2H), 2.91-2.93 (m, 1H), 2.87-2.90 (m, 2H), 2.80-2.86 (m, 2H), 2.65-2.75 (m, 1H), 2.53- 2.64 (m, 1H), 1.48-1.62 (m, 1H), 1.35-1.47 (m, 1H), 0.35-0.54 (m, 1H), 0.17-0.34 (m, 2H), −0.06- 0.06 (m, 2H). |
| 206 | 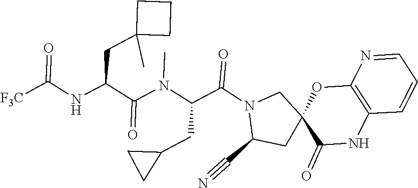 [SMILES: CN(C(=O)[C@H](CC1(C)CCC1)NC(=O)C(F)(F)F)[C@@H](CC1CC1)C(=O)N1C[C@@]2(C[C@H]1C#N)Oc1ncccc1NC2=O] | 613 [M + Na]⁺ | ¹H NMR (400 MHz, 80° C., DMSO-d₆) δ 9.45 (br, 1H), 7.75- 7.91 (m, 1H), 7.22-7.38 (m, 1H), 7.00-7.13 (m, 1H), 5.20-5.40 (m, 1H), 4.88-5.10 (m, 1H), 4.70- 4.87 (m, 1H), 4.00-4.30 (m, 2H), 3.01 (s, 3H), 2.79-2.87 (m, 1H), 2.55-2.78 (m, 1H), 1.78-2.00 (m, 3H), 1.55-1.77 (m, 5H), 1.35- 1.54 (m, 2H), 1.08 (s, 3H), 0.52- 0.70 (m, 1H), 0.25-0.45 (m, 2H), 0.05-0.13 (m, 2H). |
| 207 | 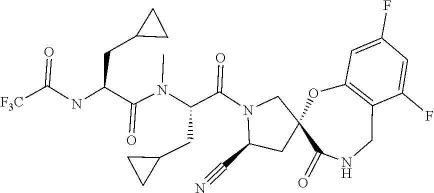 | 634 [M + Na]⁺ | ¹H NMR (400 MHz, 80° C. DMSO-d₆) δ 9.20 (s, 1H), 8.30- 8.55 (m, 1H), 6.90-7.10 (m, 2H), 5.10-5.25 (m, 1H), 4.90-5.08 (m, 1H), 4.60-4.85 (m, 1H), 4.20- 4.30 (m, 1H), 4.10-4.18 (m, 2H), 3.90-4.05 (m, 1H), 2.90-2.92 (m, 3H), 2.70-2.85 (m, 1H), 2.50- 2.60 (m, 1H), 1.50-1.65(m, 2H) 1.35-1.48 (m, 2H), 0.50-0.70 (m, 2H), 0.10-0.40 (m, 4H), 0.00- 0.01 (m, 2H), −0.20 − −0.01 (m, 2H). |
| 208 | 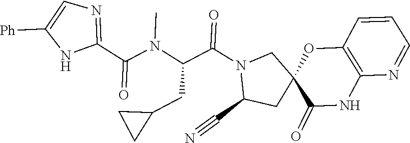 [SMILES: CN(C(=O)c1ncc(-c2ccccc2)[nH]1)[C@@H](CC1CC1)C(=O)N1C[C@@]2(C[C@H]1C#N)Oc1cccnc1NC2=O] | 526 [M + H]⁺ | ¹H NMR (400 MHz, 80° C., DMSO-d₆) δ 12.40-13.20 (m, 1H), 10.90-12.00 (m, 1H), 7.80- 8.00 (m, 2H), 7.50-7.70 (m, 2H), 7.30-7.45 (m, 2H), 7.10-7.25 (m, 2H) 6.45-7.00 (m, 1H), 4.90-5.60 (m, 1H), 3.90-4.70 (m, 2H), 3.50- 3.75 (m, 1H), 3.00-3.05 (m, 3H), 2.70-2.90 (m, 2H), 1.60-1.90 (m, 2H), 0.60-0.80 (m, 1H), 0.25- 0.50 (m, 2H), 0.10-0.20 (m, 2H) |
| 209 | 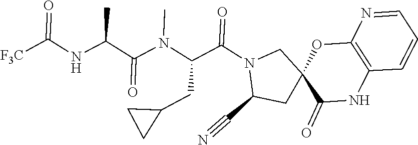 [SMILES: C[C@H](NC(=O)C(F)(F)F)C(=O)N(C)[C@@H](CC1CC1)C(=O)N1C[C@@]2(C[C@H]1C#N)Oc1ncccc1NC2=O] | 545 [M + Na]⁺ | ¹H NMR (400 MHz, 80° C., DMSO-d₆) δ 11.06 (br, 1H), 9.32 (br, 1H), 7.84-7.91 (m, 1H), 7.31- 7.39 (m, 1H), 7.00-7.13 (m, 1H), 5.18-5.29 (m, 1H), 4.96-5.05 (m, 1H), 4.62-4.78 (m, 1H), 4.01- 4.17 (m, 2H), 2.98 (s, 3H), 3.70- 3.91 (m, 2H), 1.57-1.78 (m, 2H), 1.11 (s, 3H), 0.58-0.69 (m, 1H), 0.31-0.48 (m, 2H), 0.07-0.15 (m, 2H). |
| 210 | 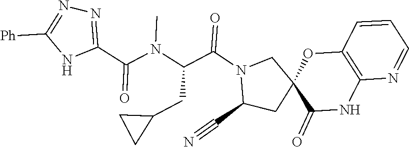 [SMILES: CN(C(=O)c1nnc(-c2ccccc2)[nH]1)[C@@H](CC1CC1)C(=O)N1C[C@@]2(C[C@H]1C#N)Oc1cccnc1NC2=O] | 527 [M + H]⁺ | ¹H NMR (400 MHz, 80° C., DMSO-d₆) δ 11.20-11.70 (m, 1H), 7.90-8.15 (m, 2H), 7.82- 7.89 (m, 1H), 7.43-7.60 (m, 3H), 7.11-7.29 (m, 1H), 6.62-6.95 (m, 1H), 6.01-6.13 (m, 0.3H), 5.20- 5.36 (m, 0.6H), 5.00-5.11 (m, 1H), 4.03-4.55 (m, 2H), 3.24- 3.30 (m, 2H), 2.76-2.92 (m, 3H), 1.72-1.80 (m, 2H), 0.64-0.70 (m, |
| | | | 1H), 0.32-0.56 (m, 2H), −0.10- |
| | | | 0.22 (m, 2H). |
| 211 | 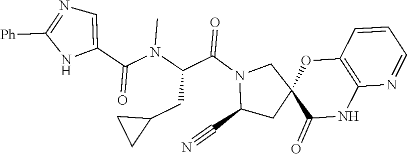 [SMILES: CN(C(=O)c1cnc(-c2ccccc2)[nH]1)[C@@H](CC1CC1)C(=O)N1C[C@@]2(C[C@H]1C#N)Oc1cccnc1NC2=O] | 526 [M + H]⁺ | ¹H NMR (400 MHz, 80° C., DMSO-d₆) δ 12.05-13.30 (m, 1H), 10.80-12.04 (m, 1H), 7.81- 8.04 (m, 3H), 7.51-7.63 (m, 1H), 7.35-7.47 (m, 3H), 7.12-7.29 (m, 1H), 6.63-7.06 (m, 1H), 4.89- 5.21 (m, 1H), 3.88 -4.63 (m, 2H), 3.19-3.50 (m, 3H), 2.72-2.87 (m, 2H), 1.78-1.97 (m, 1H), 1.51- 1.77 (m, 1H), 0.52-0.83 (m, 1H), |
| | | | 0.28-0.49 (m, 2H), 0.06-0.21 (m, |
| | | | 2H). |
| 212 | 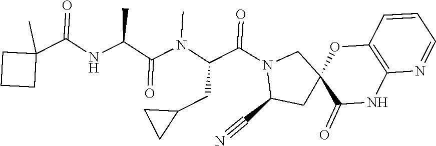 [SMILES: C[C@H](NC(=O)C1(C)CCC1)C(=O)N(C)[C@@H](CC1CC1)C(=O)N1C[C@@]2(C[C@H]1C#N)Oc1cccnc1NC2=O] | 545 [M + Na]⁺ | . ¹H NMR (400 MHz, 80° C. DMSO-d₆) δ11.4 (s, 1H), 7.90- 8.20 (m, 1H), 7.30-7.40 (m, 1H), 7.15-7.28 (m, 1H), 6.90-7.08 (m, 1H), 5.10-5.25 (m, 1H), 4.80- 5.08 (m, 1H), 4.50-4.75 (m, 1H), 3.95-4.15 (m, 2H), 2.90-3.01(m, 3H), 2.70-2.88 (m, 2H), 2.20- 2.35 (m, 2H), 1.80-1.90(m, 1H) 1.60-1.70( m, 2H), 1.55-1.58 (m, |
| | | | 3H), 1.20-1.30 (m, 3H), 0.98- |
| | | | 1.00 (m, 3H), 0.55-0.70 (m, 1H), |
| | | | 0.30-0.50(m, 2H), 0.01-0.15 (m, |
| | | | 2H). |
| 213 | 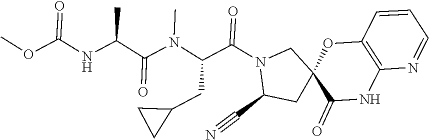 [SMILES: COC(=O)N[C@@H](C)C(=O)N(C)[C@@H](CC1CC1)C(=O)N1C[C@@]2(C[C@H]1C#N)Oc1cccnc1NC2=O] | 507 [M + Na]⁺ | ¹H NMR (400 MHz, 80° C., DMSO-d₆) δ 11.35 (br, 1H), 7.80- 7.95 (m, 1H), 7.21-7.31 (m, 1H), 6.89-6.98 (m, 1H), 6.70-6.88 (m, 1H), 4.98-5.18 (m, 1H), 4.80- 4.97 (m, 1H), 4.20-4.42 (m, 1H), 3.80-4.01 (m, 2H), 3.42 (s, 3H), 2.80-2.90 (m, 3H), 2.58-2.79 (m, 2H), 1.40-1.70 (m, 2H), 0.80- |
| | | | 1.01 (m, 3H), 0.45-0.65 (m, 1H), |
| | | | 0.20-0.43 (m, 2H), −0.05-0.10 (m, |
| | | | 2H). |
| 214 | 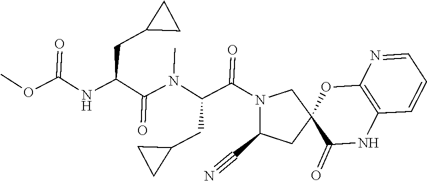 [SMILES: COC(=O)N[C@@H](CC1CC1)C(=O)N(C)[C@@H](CC1CC1)C(=O)N1C[C@@]2(C[C@H]1C#N)Oc1ncccc1NC2=O] | 525 [M + H]⁺ | ¹H NMR (400 MHz, 80° C., DMSO-d₆) δ 7.75-7.90 (m, 1H), 7.28-7.40 (m, 1H), 7.00-7.15 (m, 1H), 6.88 (br, 1H), 5.15-5.35 (m, 1H), 4.82-5.05 (m, 1H), 4.30- 4.55 (m, 1H), 4.00-4.22 (m, 2H), 3.50 (s, 3H), 2.95-3.00 (m, 3H), 2.75-2.90 (m, 1H), 2.60-2.74 (m, 1H), 1.65-1.82 (m, 1H), 1.50- 1.64 (m, 1H), 1.20-1.49 (m, 2H), 0.52-0.73 (m, 2H), 0.20-0.45 (m, 4H), 0.05-0.18 (m, |
| | | | 2H), −0.20 − −0.00 (m, 2H). |
| 215 | 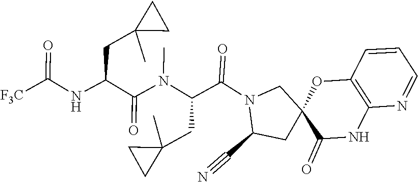 [SMILES: CN(C(=O)[C@H](CC1(C)CC1)NC(=O)C(F)(F)F)[C@@H](CC1(C)CC1)C(=O)N1C[C@@]2(C[C@H]1C#N)Oc1cccnc1NC2=O] | 591 [M + H]⁺ | ¹H NMR (400 MHz, 80° C., DMSO-d₆) δ 9.39 (br, 1H), 7.90- 8.14 (m, 1H), 7.20-7.46 (m, 1H), 6.82-7.19 (m, 1H), 5.21-5.63 (m, 1H), 4.71-5.18 (m, 2H), 3.82- 4.39 (m, 2H), 2.95-3.03 (s, 3H), 2.79-2.88 (m, 1H), 2.56-2.71 (m, 1H), 1.83-2.08 (m, 1H), 1.63- 1.82 (m, 1H), 1.49-1.62 (m, 1H), 1.31-1.48 (m, 1H), 0.82-1.12 (m, 6H), 0.41-0.56 (m, 1H), 0.27- 0.40 (m, 2H), 0.09-0.26 (m, |
| | | | 4H), −0.02-0.08 (m, 1H). |
| 216 | 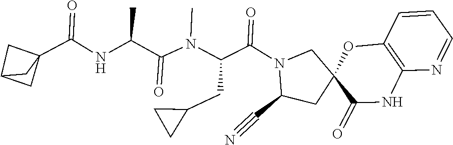 [SMILES: C[C@H](NC(=O)C12CC(C1)C2)C(=O)N(C)[C@@H](CC1CC1)C(=O)N1C[C@@]2(C[C@H]1C#N)Oc1cccnc1NC2=O] | 543 [M + Na]⁺ | ¹H NMR (400 MHz, 80° C., DMSO-d₆) δ 11.42 (s, 1H), 7.90- 8.15 (m, 1H), 7.40-7.50 (m, 1H), 7.30-7.38 (m, 1H), 6.90-7.15 (m, 1H), 5.10-5.45 (m, 1H), 4.80- 5.07 (m, 1H), 4.40-4.85 (m, 1H), 3.70-4.30 (m, 2H), 2.90-3.02 (m, 3H), 2.70-2.85 (m, 2H), 2.35- 2.40 (m, 1H), 1.90-2.05 (m, 6H), |
| | | | 1.50-1.80 (m, 2H), 0.90-1.20(m, |
| | | | 3H), 0.50-0.60 (m, 1H), 0.25- |
| | | | 0.48 (m, 2H), 0.01-0.15 (m, 2H). |
| 217 | 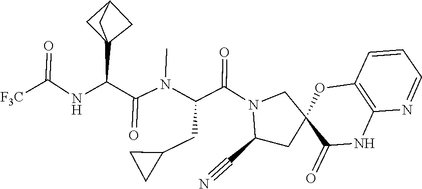 [SMILES: CN(C(=O)[C@@H](NC(=O)C(F)(F)F)C12CC(C1)C2)[C@@H](CC1CC1)C(=O)N1C[C@@]2(C[C@H]1C#N)Oc1cccnc1NC2=O] | 597 [M + Na]⁺ | ¹H NMR (400 MHz, 80° C., DMSO-d₆) δ 11.28-11.61 (m, 1H), 8.77 (br, 1H), 7.96-8.03 (m, 1H), 7.26-7.32 (m, 1H), 6.98- 7.10 (m, 1H), 5.10-5.30 (m, 1H), 4.80-5.09 (m, 2H), 4.28-4.40 (m, 1H), 4.10-4.27 (m, 1H), 3.02- 3.08 (m, 3H), 2.79-2.90 (m, 1H), 2.60-2.78 (m, 1H), 2.35-2.45 (m, 1H), 1.79-1.90 (m, 3H), 1.50- 1.78 (m, 5H), 0.55-0.80 (m, 1H), 0.30-0.48 (m, 2H), 0.02-0.20 (m, |
| | | | 2H). |
| 218 | 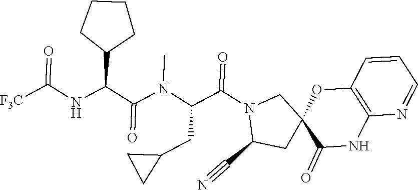 [SMILES: CN(C(=O)[C@@H](NC(=O)C(F)(F)F)C1CCCC1)[C@@H](CC1CC1)C(=O)N1C[C@@]2(C[C@H]1C#N)Oc1cccnc1NC2=O] | 599 [M + Na]⁺ | ¹H NMR (400 MHz, 80° C., DMSO-d₆) δ 11.43 (br, 1H), 9.40 (br, 1H), 7.90-8.00 (m, 1H), 7.22- 7.38 (m, 1H), 6.90-7.10 (m, 1H), 5.15-5.30 (m, 1H), 4.88-5.13 (m, 1H), 4.50-4.68 (m, 1H), 3.98- 4.25 (m, 2H), 2.97- 3.02 (m, 3H), 2.70-2.88 (m, 1H), 2.55-2.69 (m, 1H), 2.28-2.38 (m, 1H), 1.52- 1.75 (m, 3H), 1.30-1.51 (m, 5H), 1.10-1.29 (m, 1H), 0.89-1.09 (m, 1H), 0.51-0.70 (m, 1H), 0.22- |
| | | | 0.45 (m, 2H), 0.00-0.13 (m, 2H). |
| 219 | 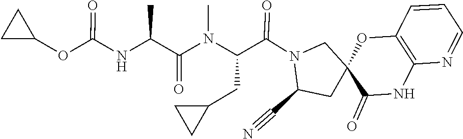 [SMILES: C[C@H](NC(=O)OC1CC1)C(=O)N(C)[C@@H](CC1CC1)C(=O)N1C[C@@]2(C[C@H]1C#N)Oc1cccnc1NC2=O] | 533 [M + Na]⁺ | ¹H NMR (400 MHz, 80° C., DMSO-d₆) δ 11.30 (br, 1H), 7.80- 7.98 (m, 1H), 7.15-7.30 (m, 1H), 6.89-7.00 (m, 1H), 6.70-6.88 (m, 1H), 4.99-5.25 (m, 1H), 4.72- 4.98 (m, 1H), 4.20-4.40 (m, 1H), 3.98-4.05 (m, 2H), 3.70-3.97 (m, 1H), 2.86 (s, 3H), 2.55-2.80 (m, 2H), 1.40-1.64 (m, 2H), 0.75- |
| | | | 1.00 (m, 3H), 0.45-0.70 (m, 3H), |
| | | | 0.38-0.44 (m, 2H), 0.21-0.37 (m, |
| | | | 2H), −0.04-0.12 (m, 2H). |
| 220 | 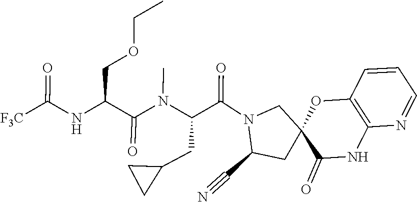 [SMILES: CCOC[C@H](NC(=O)C(F)(F)F)C(=O)N(C)[C@@H](CC1CC1)C(=O)N1C[C@@]2(C[C@H]1C#N)Oc1cccnc1NC2=O] | 589 [M + Na]⁺ | ¹H NMR (400 MHz, 80° C., DMSO-d₆) δ 11.7 (s, 1H), 9.75 (s, 1H), 7.90-8.10 (m, 1H), 7.40- 7.50 (m, 1H), 6.95-7.13 (m, 1H), 5.10-5.25 (m, 1H), 4.90-5.08 (m, 1H), 4.80-4.88 (m, 1H), 3.98- 4.18 (m, 2H), 3.45-3.50 (m, 2H), 3.30-3.42 (m, 2H), 3.00-3.05(m, 3H) 2.70-2.90 (m, 1H), 2.60-2.68 (m, 1H), 1.50-1.75 (m, 2H), 1.00- 1.10 (m, 3H), 0.50-0.65 (m, 1H), 0.20-0.45(m, 2H), 0.01-0.15 (m, 2H). |
| 221 | 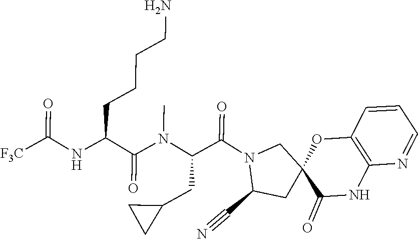 [SMILES: CN(C(=O)[C@H](CCCCN)NC(=O)C(F)(F)F)[C@@H](CC1CC1)C(=O)N1C[C@@]2(C[C@H]1C#N)Oc1cccnc1NC2=O] | 580 [M + H]⁺ | ¹H NMR (400 MHz, 80° C., DMSO-d₆) δ 7.95-8.05 (m, 1H), 7.25-7.31 (m, 1H), 6.95-7.05 (m, 1H), 5.10-5.25 (m, 1H), 4.95- 5.05 (m, 1H), 4.55-4.65 (m, 1H), 4.05-4.15 (m, 2H), 3.00 (s, 3H), 2.72-2.85 (m, 1H), 2.60-2.71 (m, 1H), 2.38-2.45 (m, 2H), 1.48- 1.75 (m, 4H), 1.10-1.40 (m, 4H), 0.50-0.70 (m, 1H), 0.23-0.43 (m, 2H), 0.00-0.20 (m, 2H). |

ph = phenyl

### Example 73

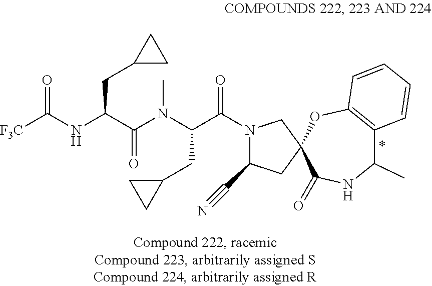

(2S)—N-((2S)-1-((2R,5'S)-5′-cyano-5-methyl-3-oxo-4,5-dihydro-3H-spiro[benzo[f][1,4]oxazepine-2,3′-pyrrolidin]-1′-yl)-3-cyclopropyl-1-oxopropan-2-yl)-3-cyclopropyl-N-methyl-2-(2,2,2-trifluoroacetamido)propanamide (31.3 mg, 39%) was obtained as a white solid. ¹H NMR (400 MHz, 80° C. DMSO-d₆) δ 9.45 (s, 1H), 8.30-8.55 (m, 1H), 7.30-7.45 (m, 2H), 7.10-7.28 (m, 2H), 5.20-5.50 (m, 1H), 5.00-5.18 (m, 1H), 4.50-4.98 (m, 2H), 3.80-4.40 (m, 2H), 3.00-3.05 (m, 3H), 2.70-2.85 (m, 1H), 2.50-2.53 (m, 1H), 1.40-1.85 (m, 7H), 0.60-0.90 (m, 2H), 0.20-0.50 (m, 4H), 0.10-0.18 (m, 2H), −0.20-−0.01 (m, 2H). LC-MS (ESI, m/z): 612 [M+Na]⁺.

(2S)—N-((2S)-1-((2R,5'S)-5′-cyano-5-methyl-3-oxo-4,5-dihydro-3H-spiro[benzo[f][1,4]oxazepine-2,3′-pyrrolidin]-1′-yl)-3-cyclopropyl-1-oxopropan-2-yl)-3-cyclopropyl-N-methyl-2-(2,2,2-trifluoroacetamido)propenamide (Compound 222) was synthesized similarly as described for (S)—N—((S)-1-((2R,5'S)-5′-cyano-3-oxo-4,5-dihydro-3H-spiro[benzo[f][1,4]oxazepine-2,3′-pyrrolidin]-1′-yl)-3-cyclopropyl-1-oxopropan-2-yl)-3-cyclopropyl-N-methyl-2-(2,2,2-trifluoroacetamido)propenamide (Compound 81) where t-butyl (1-(2-hydroxyphenyl)ethyl)carbamate was used in place of t-butyl (2-hydroxybenzyl)carbamate.

To a solution of 2-(1-aminoethyl)phenol (2.00 g, 14.6 mmol) in DCM (30 mL) were added di-tert-butyl dicarbonate (4.77 g, 21.9 mmol) and triethylamine (7.38 g, 72.9 mmol). The mixture was stirred for 1 h at rt and the reaction was quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product that was chromatographed on a silica gel column with MeOH:DCM (1:99) to provide t-butyl (1-(2-hydroxyphenyl)ethyl)carbamate (3 g, 86%) as a white solid. ¹H NMR (400 MHz, DMSO-d₆) δ 9.38 (s, 1H), 7.11-7.29 (m, 2H), 6.98-7.03 (m, 1H), 6.66-6.81 (m, 2H), 4.79-4.96 (m, 1H), 1.29-1.43 (m, 9H), 1.17-1.23 (m, 3H). LC-MS (ESI, m/z): 182 [M-56]⁺. Prep-SFC-HPLC column separation (Column: CHIRAL ART Cellulose-SB, 2*25 cm, 5 μm; Mobile Phase A: Hex (0.1% FA)-HPLC, Mobile Phase B: IPA: DCM; Flow rate: 20 ML/MIN mL/min; Gradient: isocratic 15; Wave Length: 254/220 nm; RT1(min): 11.991; RT2(min): 13.926; Sample Solvent: EtOH; Injection Volume: 0.4 mL;). Purification resulted in Compounds 223 and 224 (*Temporary assignment).

Compound 223: ((S)—N—((S)-1-((2R,5S*,5'S)-5′-cyano-5-methyl-3-oxo-4,5-dihydro-3H-spiro[benzo[f][1,4]oxazepine-2,3′-pyrrolidin]-1′-yl)-3-cyclopropyl-1-oxopropan-2-yl)-3-cyclopropyl-N-methyl-2-(2,2,2-trifluoroacetamido)propanamide) (6.5 mg, 30%) as white solid. ¹H NMR (400 MHz, 80° C., DMSO-d₆) δ 9.35 (s, 1H), 8.35 (s, 1H), 7.30-7.40 (m, 2H), 7.15-7.28 (m, 1H), 7.10-7.14 (m, 1H), 5.28 (br, 1H), 5.00-5.18 (m, 1H), 4.60-4.90 (m, 2H), 4.10-4.35 (m, 1H), 3.80-4.08 (m, 1H), 3.00-3.02 (m, 3H), 2.60-2.85 (m, 2H), 1.60-1.80 (m, 3H), 1.40-1.58 (m, 4H), 0.50-0.80 (m, 2H), 0.30-0.48 (m, 2H), 0.10-0.28 (m, 2H), 0.00-0.08 (m, 2H), −0.02-−0.01 (m, 2H). LC-MS (ESI, m/z): 612 [M+Na]⁺.

Compound 224 ((S)—N—((S)-1-((2R,5R*,5'S)-5′-cyano-5-methyl-3-oxo-4,5-dihydro-3H-spiro[benzo[f][1,4]oxazepine-2,3′-pyrrolidin]-1′-yl)-3-cyclopropyl-1-oxopropan-2-yl)-3-cyclopropyl-N-methyl-2-(2,2,2-trifluoroacetamido)propanamide) as white solid. (6.7 mg, 30%) as white solid. ¹H NMR (400 MHz, 80° C., DMSO-d₆) δ 9.40 (s, 1H), 8.45 (s, 1H), 7.25-7.40 (m, 2H), 7.15-7.20 (m, 1H), 7.00-7.13 (m, 1H), 5.29 (br, 1H), 4.90-5.15 (m, 1H), 4.70-4.88 (m, 1H), 4.40-4.68 (m, 1H), 3.95-4.30 (m, 2H), 3.10-3.15 (m, 3H), 2.60-2.80 (m, 1H), 2.50-2.55 (m, 1H), 1.60-1.80 (m, 4H), 1.40-1.58 (m, 3H), 0.55-0.80 (m, 2H), 0.28-0.50 (m, 3H), 0.15-0.23 (m, 1H), 0.01-0.14 (m, 2H), −0.10-−0.01 (m, 2H). LC-MS (ESI, m/z): 612 [M+Na]⁺.

### Example 74

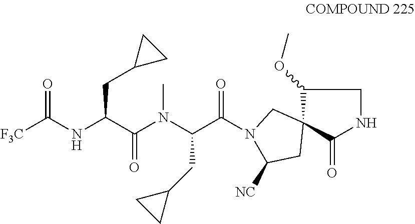

To a solution of 2-(tert-butyl) 3-methyl (3S)-9-hydroxy-7-(4-methoxybenzyl)-6-oxo-2,7-diazaspiro[4.4]nonane-2,3-dicarboxylate (2.0 g, 4.60 mmol) in DMF (20 mL) were added sodium hydride (133 mg, 5.52 mmol) and CH₃I (980 mg, 6.90 mmol) stirred under nitrogen at 0° C. The mixture was stirred for 2 h at rt and the reaction was quenched with water (100 mL). The mixture was extracted with EA (3×200 mL). The organic layers were combined, washed with brine (2×200 mL), dried over anhydrous sodium sulfate, filtered and concentrated under reduced pressure to afford the crude product. The crude product was purified by TLC (Mobile phase: ethyl acetate/petroleum ethe=1:1.5; Rf=0.4; detection: UV) to provide 2-(tert-butyl) 3-methyl (3S,5S)-9-methoxy-7-(4-methoxybenzyl)-6-oxo-2,7-diazaspiro[4.4]nonane-2,3-dicarboxylate (600 mg, 29%) as a yellow semi-solid. ¹H NMR (400 MHz, DMSO-d₆) δ 7.06-7.18 (m, 2H), 6.87-6.97 (m, 2H), 4.11-4.44 (m, 3H), 3.72-3.80 (m, 4H), 3.63-3.71 (m, 3H), 3.48-3.59 (m, 1H), 3.38-3.47 (m, 1H), 3.09-3.30 (m, 5H), 2.53-2.61 (m, 0.6H), 2.33-2.44 (m, 0.4H), 2.22-2.27 (m, 0.4H), 1.86-2.01 (m, 0.6H), 1.31-1.44 (m, 9H). LC-MS (ESI, m/z): 449 [M+H]⁺.

To a solution of 2-(tert-butyl) 3-methyl (3S,5S)-9-methoxy-7-(4-methoxybenzyl)-6-oxo-2,7-diazaspiro[4.4]nonane-2,3-dicarboxylate (600 mg, 1.34 mmol) in THF (6 mL) was added NaOH (214 mg, 5.35 mmol, in 3 mL H₂O). The mixture was stirred overnight at rt. The mixture was concentrated under reduced pressure to remove THF and acidified to pH=5 with HCl (1 M). The mixture was extracted with EA (3×100 mL). The organic layers were combined, dried over anhydrous sodium sulfate, filtered and concentrated under reduced pressure to provide (3S,5S)-2-(tert-butoxycarbonyl)-9-methoxy-7-(4-methoxybenzyl)-6-oxo-2,7-diazaspiro[4.4]nonane-3-carboxylic acid (520 mg, crude) as a yellow solid. ¹H NMR (400 MHz, DMSO-d₆) δ 12.86 (br, 1H), 7.08-7.18 (m, 2H), 6.85-6.96 (m, 2H), 4.03-4.44 (m, 3H), 3.66-3.78 (m, 4H), 3.39-3.58 (m, 2H), 3.11-3.27 (m, 5H), 2.54-2.61 (m, 0.6H), 2.35-2.43 (m, 0.4H), 2.25-2.32 (m, 0.4H), 1.92-2.02 (m, 0.6H), 1.32-1.47 (m, 9H). LC-MS (ESI, m/z): 891 [2M+Na]⁺.

To a solution of (3S,5S)-2-(tert-butoxycarbonyl)-9-methoxy-7-[(4-methoxyphenyl)methyl]-6-oxo-2,7-diazaspiro[4.4]nonane-3-carboxylic acid (520 mg, 1.20 mmol), ammonium chloride (480 mg, 8.98 mmol) and HATU (1.00 g, 2.64 mmol) in DMF (10 mL) was added N-ethyl-N-isopropylpropan-2-amine (1.55 g, 12.0 mmol) stirred at 0° C. The mixture was stirred for 1 h at rt and the reaction was quenched with water (10 mL). The mixture was purified by C18 column with CH₃CN/Water (0.05% TFA). The fraction was concentrated under reduced pressure to provide tert-butyl (3S,5S)-3-carbamoyl-9-methoxy-7-(4-methoxybenzyl)-6-oxo-2,7-diazaspiro[4.4]nonane-2-carboxylate (450 mg, crude) as a yellow semi-solid. ¹H NMR (400 MHz, DMSO-d₆) δ 7.31-7.50 (m, 1H), 7.07-7.17 (m, 2H), 6.85-6.96 (m, 2H), 4.17-4.44 (m, 3H), 3.66-3.76 (m, 4H), 3.34-3.55 (m, 2H), 3.08-3.30 (m, 5H), 2.11-2.30 (m, 1H), 1.81-1.99 (m, 1H), 1.25-1.47 (m, 9H). LC-MS (ESI, m/z): 889 [2M+Na]⁺.

To a solution of tert-butyl (3S,5S)-3-carbamoyl-9-methoxy-7-(4-methoxybenzyl)-6-oxo-2,7-diazaspiro[4.4]nonane-2-carboxylate (450 mg, 0.923 mmol) in DCM (6 mL) was added trifluoroacetic acid (2 mL) stirred at rt. The mixture was stirred for 1 h at rt and then concentrated under reduced pressure to afford (3S,5S)-9-methoxy-7-(4-methoxybenzyl)-6-oxo-2,7-diazaspiro[4.4]nonane-3-carboxamide (350 mg, crude) as a brown semi-solid. LCMS (ESI, m/z):334 [M+H]⁺.

To a solution of (3S,5S)-9-methoxy-7-(4-methoxybenzyl)-6-oxo-2,7-diazaspiro[4.4]nonane-3-carboxamide (350 mg, 1.01 mmol), (S)-2-((tert-butoxycarbonyl)(methyl)amino)-3-cyclopropylpropanoic acid (270 mg, 1.11 mmol) and HATU(461 mg, 1.21 mmol) in DMF (5 mL) was added N-ethyl-N-isopropylpropan-2-amine (651 mg, 5.05 mmol) stirred at 0° C. The mixture was stirred for 1 h at rt and the reaction was quenched with water (80 mL). The mixture was extracted with EA (3×120 mL). The organic layers were combined, washed with brine (2×100 mL), dried over anhydrous sodium sulfate, filtered and concentrated under reduced pressure to afford the crude product. The crude product was purified by C18 column with CH₃CN/Water (0.05% TFA), (47%). The fraction was concentrated under reduced pressure to provide tert-butyl ((2S)-1-((3S,5S)-3-carbamoyl-9-methoxy-7-(4-methoxybenzyl)-6-oxo-2,7-diazaspiro[4.4]nonan-2-yl)-3-cyclopropyl-1-oxopropan-2-yl)(methyl)carbamate (420 mg, 74%) as an off-white solid. ¹H NMR (400 MHz, DMSO-d₆) δ 7.27-7.44 (m, 1H), 6.82-7.18 (m, 4H), 4.64-4.90 (m, 1H), 4.08-4.61 (m, 3H), 3.76-4.00 (m, 2H), 3.60-3.75 (m, 5H), 3.45-3.59 (m, 2H), 3.10-3.42 (m, 5H), 2.62-2.73 (m, 1H), 2.15-2.39 (m, 1H), 1.47-1.94 (m, 2H), 1.21-1.45 (m, 9H), 0.48-0.66 (m, 1H), 0.23-0.45 (m, 2H), 0.00-0.16 (m, 2H). LC-MS (ESI, m/z): 559 [M+H]⁺.

To a solution of t-butyl ((2S)-1-((3S,5S)-3-carbamoyl-9-methoxy-7-(4-methoxybenzyl)-6-oxo-2,7-diazaspiro[4.4]nonan-2-yl)-3-cyclopropyl-1-oxopropan-2-yl)(methyl)carbamate (420 mg, 0.752 mmol) in DCM (5 mL) was added trifluoroacetic acid (1.5 mL) stirred at rt. The mixture was stirred for 0.5 h at rt. The mixture was concentrated under reduced pressure to afford (3S,5S)-2-((S)-3-cyclopropyl-2-(methylamino)propanoyl)-9-methoxy-7-(4-methoxybenzyl)-6-oxo-2,7-diazaspiro[4.4]nonane-3-carboxamide (320 mg, crude) as a brown semi-solid. LCMS (ESI, m/z):459 [M+H]⁺.

To a solution of (3S,5S)-2-((S)-3-cyclopropyl-2-(methylamino)propanoyl)-9-methoxy-7-(4-methoxybenzyl)-6-oxo-2,7-diazaspiro[4.4]nonane-3-carboxamide (320 mg, 0.698 mmol), (2S)-2-[(tert-butoxycarbonyl)amino]-3-cyclopropylpropanoic acid (176 mg, 0.768 mmol) and HATU (318 mg, 0.838 mmol) in DMF (5 mL) was added N-ethyl-N-isopropylpropan-2-amine (361 mg, 2.79 mmol) stirred at 0° C. The mixture was stirred for 1 h at rt and the reaction was quenched with water (100 mL). The mixture was extracted with EA (3×150 mL). The organic layers were combined, washed with brine (2×100 mL), dried over anhydrous sodium sulfate, filtered and concentrated under reduced pressure to afford the crude product. The crude product was chromatographed on a silica gel column with MeOH:DCM (4%) to provide tert-butyl ((2S)-1-(((2S)-1-((3S,5S)-3-carbamoyl-9-methoxy-7-(4-methoxybenzyl)-6-oxo-2,7-diazaspiro[4.4]nonan-2-yl)-3-cyclopropyl-1-oxopropan-2-yl)(methyl)amino)-3-cyclopropyl-1-oxopropan-2-yl)carbamate (370 mg, 79%) as a white solid.

¹H NMR (400 MHz, DMSO-d₆) δ 7.25-7.42 (m, 1H), 7.06-7.17 (m, 2H), 6.85-7.02 (m, 3H), 5.09-5.37 (m, 1H), 4.23-4.48 (m, 3H), 3.94-4.18 (m, 1H), 3.80-3.92 (m, 1H), 3.65-3.78 (m, 3H), 3.34-3.63 (m, 3H), 3.06-3.26 (m, 4H), 2.75-3.03 (m, 3H), 2.31-2.48 (m, 1H), 2.07-2.26 (m, 1H), 1.28-1.62 (m, 13H), 0.58-0.83 (m, 2H), 0.21-0.47 (m, 4H), −0.02-0.16 (m, 4H). LC-MS (ESI, m/z): 692 [M+Na]⁺.

To a solution of tert-butyl ((2S)-1-(((2S)-1-((3S,5S)-3-carbamoyl-9-methoxy-7-(4-methoxybenzyl)-6-oxo-2,7-diazaspiro[4.4]nonan-2-yl)-3-cyclopropyl-1-oxopropan-2-yl)(methyl)amino)-3-cyclopropyl-1-oxopropan-2-yl)carbamate (370 mg, 553 mmol) in DCM (4.5 mL) was added trifluoroacetic acid (1.5 mL) stirred at rt. The mixture was stirred for 1 h at rt and then concentrated under reduced pressure to afford (3S,5S)-2-((S)-2-((S)-2-amino-3-cyclopropyl-N-methylpropanamido)-3-cyclopropylpropanoyl)-9-methoxy-7-(4-methoxybenzyl)-6-oxo-2,7-diazaspiro[4.4]nonane-3-carboxamide (280 mg, crude) as a brown semi-solid. LCMS (ESI, m/z):570 [M+H]⁺.

To a mixture of (3S,5S)-2-((S)-2-((S)-2-amino-3-cyclopropyl-N-methylpropanamido)-3-cyclopropylpropanoyl)-9-methoxy-7-(4-methoxybenzyl)-6-oxo-2,7-diazaspiro[4.4]nonane-3-carboxamide (280 mg, 0.491 mmol) in MeOH (3 mL) were added triethylamine (598 mg, 5.89 mmol) and ethyl 2,2,2-trifluoroacetate (1.07 g, 4.91 mmol). The mixture was stirred for 1 h at rt. The reaction was quenched with water (50 mL) and adjusted acid to pH=4 with HCl (1M). The mixture was extracted with EA (3×80 mL). The organic layers were combined, washed with brine (2×50 mL), dried over anhydrous sodium sulfate, filtered and concentrated under reduced pressure to afford the crude product. The crude product was purified by TLC (Mobile phase: MeOH/DCM=1:15; Rf=0.4; detection: UV) to provide (3S,5S)-2-((S)-3-cyclopropyl-2-((S)-3-cyclopropyl-N-methyl-2-(2,2,2-trifluoroacetamido)propanamido)propanoyl)-9-methoxy-7-(4-methoxybenzyl)-6-oxo-2,7-diazaspiro[4.4]nonane-3-carboxamide (230 mg, 70%) as a yellow solid. ¹H NMR (400 MHz, DMSO-d₆) δ 9.63-9.82 (m, 1H), 7.04-7.43 (m, 3H), 6.82-7.02 (m, 3H), 5.09-5.31 (m, 1H), 4.65-4.88 (m, 1H), 4.10-4.44 (m, 3H), 3.82-4.00 (m, 1H), 3.70-3.78 (m, 3H), 3.50-3.63 (m, 1H), 3.32-3.45 (m, 3H), 3.09-3.24 (m, 4H), 2.92-3.06 (m, 2H), 2.46-2.49 (m, 1H), 2.13-2.21 (m, 1H), 1.39-1.76 (m, 4H), 0.53-0.78 (m, 2H), 0.26-0.47 (m, 4H), 0.11-0.23 (m, 1H), 0.00-0.11 (m, 3H). LC-MS (ESI, m/z): 666 [M+H]⁺.

To a solution of (3S,5S)-2-((S)-3-cyclopropyl-2-((S)-3-cyclopropyl-N-methyl-2-(2,2,2-trifluoroacetamido)propanamido)propanoyl)-9-methoxy-7-(4-methoxybenzyl)-6-oxo-2,7-diazaspiro[4.4]nonane-3-carboxamide (200 mg, 0.300 mmol) in ACN (4 mL) and H₂O (1 mL) was added ammonium ceric nitrate (496 mg, 0.900 mmol) stirred at rt. The mixture was stirred for 2 h at rt and then diluted with water (50 mL). The mixture was extracted with EA (3×100 mL). The organic layers were combined, washed with brine (2×50 mL), dried over anhydrous sodium sulfate, filtered and concentrated under reduced pressure to afford the crude product. The crude product was chromatographed on a silica gel column with MeOH:DCM (12%-15%) to provide (3S,5S)-2-((S)-3-cyclopropyl-2-((S)-3-cyclopropyl-N-methyl-2-(2,2,2-trifluoroacetamido)propanamido)propanoyl)-9-methoxy-6-oxo-2,7-diazaspiro[4.4]nonane-3-carboxamide (110 mg, 67%) as a light yellow solid. ¹H NMR (400 MHz, DMSO-d₆) δ 9.74 (br, 1H), 7.79-7.96 (m, 1H), 7.25-7.44 (m, 1H), 6.77-6.96 (m, 1H), 5.11-5.32 (m, 1H), 4.63-4.88 (m, 1H), 4.05-4.33 (m, 1H), 3.82-3.91 (m, 1H), 3.45-3.60 (m, 2H), 3.34-3.44 (m, 2H), 3.09-3.28 (m, 4H), 2.99-3.06 (m, 1H), 2.86-2.94 (m, 1H), 2.42-2.45 (m, 1H), 2.08-2.14 (m, 1H), 1.43-1.72 (m, 4H), 0.51-0.79 (m, 2H), 0.24-0.48 (m, 4H), 0.14-0.23 (m, 1H), 0.00-0.11 (m, 3H). LC-MS (ESI, m/z): 568 [M+Na]⁺.

To a solution of (3S,5S)-2-[(2S)-3-cyclopropyl-2-[(2S)-3-cyclopropyl-N-methyl-2-(2,2,2-trifluoroacetamido)propanamido]propanoyl]-9-methoxy-6-oxo-2,7-diazaspiro[4.4]nonane-3-carboxamide (90 mg, 0.165 mmol) in DCM (2 mL) were added pyridine (78.3 mg, 0.990 mmol) and trifluoroacetic anhydride (62.4 mg, 0.297 mmol) stirred at rt. The mixture was stirred for 1 h at rt and the reaction was quenched with water (15 mL). The mixture was extracted with DCM (3×30 mL). The organic layers were combined, washed with brine (2×20 mL), dried over anhydrous sodium sulfate, filtered and concentrated under reduced pressure to afford the crude product. The crude product was purified by prep-HPLC (Column: Xselect CSH Prep C18 Column, 30*150 mm, 5 m; Mobile Phase A: Water(0.1% FA), Mobile Phase B: ACN; Flow rate: 60 mL/min mL/min; Gradient: 29% B to54% B in 10 min; Wave Length: 254 nm/220 nm; RT1(min): 8.68) to provide (2S)—N-((2S)-1-((3S,5S)-3-cyano-9-methoxy-6-oxo-2,7-diazaspiro[4.4]nonan-2-yl)-3-cyclopropyl-1-oxopropan-2-yl)-3-cyclopropyl-N-methyl-2-(2,2,2-trifluoroacetamido)propanamide (41.1 mg, 46%) as a white solid. ¹H NMR (400 MHz, DMSO-d₆) δ 9.40-9.50 (m, 1H), 7.72-7.86 (m, 1H), 5.13-5.30 (m, 1H), 4.60-4.86 (m, 2H), 3.85-3.96 (m, 1H), 3.64-3.73 (m, 1H), 3.54-3.63 (m, 1H), 3.43-3.51 (m, 1H), 3.38-3.40 (m, 1H), 3.32-3.37 (m, 2H), 3.19-3.20 (m, 1H), 3.12-3.15 (m, 2H), 2.90-3.00 (m, 1H), 2.65-2.79 (m, 0.6H), 2.45-2.51 (m, 0.5H), 2.21-2.31 (m, 0.4H), 2.00-2.10 (m, 0.6H), 1.56-1.80 (m, 4H), 0.53-0.74 (m, 2H), 0.24-0.51 (m, 4H), 0.00-0.20 (m, 4H). LC-MS (ESI, m/z): 550 [M+Na]⁺.

### Example 75

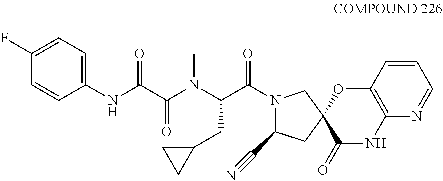 [SMILES: CN(C(=O)C(=O)Nc1ccc(F)cc1)[C@@H](CC1CC1)C(=O)N1C[C@@]2(C[C@H]1C#N)Oc1cccnc1NC2=O]

To a mixture of t-butyl ((S)-1-((2R,5'S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)(methyl)carbamate (120 mg, 0.253 mmol) in DCM (3 mL) was added trifluoroacetic acid (1 mL). The mixture was stirred for 1 h at rt and then concentrated under reduced pressure to afford (2R,5'S)-1′-((S)-3-cyclopropyl-2-(methylamino)propanoyl)-3-oxo-3,4-dihydrospiro[pyrido[3,2-b][1,4]oxazine-2,3′-pyrrolidine]-5′-carboxamide (95 mg, crude) as a light brown semi-solid. LC-MS (ESI, m/z): 374 [M+H]⁺.

To a mixture of (2R,5'S)-1′-((S)-3-cyclopropyl-2-(methylamino)propanoyl)-3-oxo-3,4-dihydrospiro[pyrido[3,2-b][1,4]oxazine-2,3′-pyrrolidine]-5′-carboxamide (95 mg, 0.254 mmol), 2-((4-fluorophenyl)amino)-2-oxoacetic acid (47.0 mg, 0.254 mmol) and HATU (116 mg, 0.305 mmol) in DMF (3 mL) was added DIEA (197 mg, 1.52 mmol) at 0° C. The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product, which was purified by TLC (Mobile phase: MeOH/DCM=1:11; Rf=0.3; detection: UV) to provide N¹—((S)-1-((2R,5'S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)-N²-(4-fluorophenyl)-N¹-methyloxalamide (105 mg, 65%) as a white solid.

To a mixture of N¹—((S)-1-((2R,5'S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)-N²-(4-fluorophenyl)-N¹-methyloxalamide (105 mg, 0.195 mmol) in DCM (2 mL) were added pyridine (77.0 mg, 0.975 mmol) and trifluoroacetic anhydride (82.0 mg, 0.390 mmol). The mixture was stirred for 1 h at rt and the reaction was quenched with water (5 mL). The mixture was extracted with DCM (3×5 mL). The organic layers were combined, washed with brine (2×5 mL), dried over anhydrous sodium sulfate, filtered and concentrated under reduced pressure to afford the crude product, which was purified by prep-HPLC (Column: Xselect CSH Prep C18 Column, 30×150 mm, 5 m; Mobile Phase A: Water (0.1% FA), Mobile Phase B: ACN; Flow rate: 60 mL/min; Gradient: 35% B to 56% B in 10 min; Wave Length: 254 nm/220 nm; RT1(min): 8.68) to provide N¹—((S)-1-((2R,5'S)-5′-cyano-3-oxo-3,4-dihydrospiro[pyrido[3,2-b][1,4]oxazine-2,3′-pyrrolidin]-1′-yl)-3-cyclopropyl-1-oxopropan-2-yl)-N²-(4-fluorophenyl)-N¹-methyloxalamide (36.6 mg, 35%) as a white solid. ¹H NMR (400 MHz, DMSO-d₆) δ 11.33 (br, 1H), 10.33-10.68 (m, 1H), 7.87-8.04 (m, 1H), 7.44-7.70 (m, 2H), 7.25-7.36 (m, 1H), 7.04-7.20 (m, 2H), 6.85-7.03 (m, 1H), 4.89-5.29 (m, 2H), 3.91-4.32 (m, 2H), 3.08-3.14 (m, 2H), 2.95-3.04 (m, 1H), 2.70-2.90 (m, 2H), 1.59-1.92 (m, 2H), 0.54-0.82 (m, 1H), 0.28-0.53 (m, 2H), 0.04-0.23 (m, 2H). LC-MS (ESI, m/z): 543[M+Na]⁺.

### Example 76

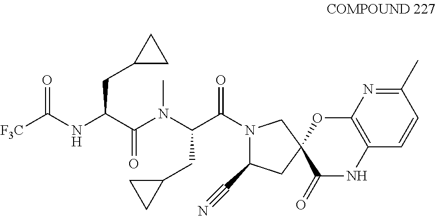 [SMILES: Cc1ccc2c(n1)O[C@@]1(C[C@@H](C#N)N(C(=O)[C@H](CC3CC3)N(C)C(=O)[C@H](CC3CC3)NC(=O)C(F)(F)F)C1)C(=O)N2]

To a mixture of 1-(tert-butyl) 2,4-dimethyl (2S)-4-hydroxypyrrolidine-1,2,4-tricarboxylate (1.00 g, 3.30 mmol) in THF (20 mL) was added sodium bis(trimethylsilyl)amide (1.65 mL, 2M in THF) at −78° C. under nitrogen. 2-fluoro-6-methyl-3-nitropyridine (1.00 g, 3.30 mmol) in THF (5 mL) was then added at −78° C. The mixture was stirred at −78° C. to rt for 2 h under nitrogen and the reaction was quenched with ammonium chloride (aq., 30 mL). The mixture was extracted with EA (3×60 mL). The organic layers were combined, washed with brine (2×30 mL), dried over anhydrous sodium sulfate, filtered and concentrated under reduced pressure to afford the crude product. The crude product was chromatographed on a silica gel column with EA:PE (30:70) to provide 1-(t-butyl) 2,4-dimethyl (2S)-4-((6-methyl-3-nitropyridin-2-yl)oxy)pyrrolidine-1,2,4-tricarboxylate (720 mg, 49%) as a red oil. ¹H NMR (400 MHz, DMSO-d₆) δ 8.37-8.51 (m, 1H), 7.01-7.32 (m, 1H), 4.35-4.72 (m, 1H), 4.21-4.39 (m, 1H), 3.73-4.20 (m, 1H), 3.57-3.72 (m, 6H), 2.82-3.06 (m, 1H), 2.52-2.70 (m, 1H), 2.43-2.49 (m, 3H), 1.32-1.45 (m, 9H). LC-MS (ESI, m/z): 462 [M+Na]⁺.

To a mixture of 1-(tert-butyl) 2,4-dimethyl (2S)-4-((6-methyl-3-nitropyridin-2-yl)oxy)pyrrolidine-1,2,4-tricarboxylate (720 mg, 1.64 mmol) in methanol (7 mL) were added iron (460 mg, 8.20 mmol) and ammonium chloride (212 mg, 3.94 mmol, aq., 1.75 mL). The mixture was stirred overnight at 60° C. under nitrogen. The mixture was filtered through a celite pad and washed with EA (3×20 mL). The reaction was quenched with water (20 mL) and the mixture was extracted with EA (3×100 mL). The organic layers were combined, washed with brine (2×20 mL), dried over anhydrous sodium sulfate, filtered and concentrated under reduced pressure to afford 1′-(tert-butyl) 5′-methyl (5'S)-6-methyl-2-oxo-1,2-dihydrospiro[pyrido[2,3-b][1,4]oxazine-3,3′-pyrrolidine]-1′,5′-dicarboxylate (600 mg, crude) as a red oil. LC-MS (ESI, m/z): 322 [M+H-56]⁺.

A mixture of 1′-(tert-butyl) 5′-methyl (5'S)-6-methyl-2-oxo-1,2-dihydrospiro[pyrido[2,3-b][1,4]oxazine-3,3′-pyrrolidine]-1′,5′-dicarboxylate (600 mg, 1.59 mmol) and ammonia (30 mL, 7 M in MeOH) was stirred for 2 d at 50° C. in a sealed vessel. The mixture was then concentrated under reduced pressure to afford t-butyl (5'S)-5′-carbamoyl-6-methyl-2-oxo-1,2-dihydrospiro[pyrido[2,3-b][1,4]oxazine-3,3′-pyrrolidine]-1′-carboxylate (400 mg, crude) as a red solid. LC-MS (ESI, m/z): 363 [M+H]⁺.

To a stirred mixture of t-butyl (5'S)-5′-carbamoyl-6-methyl-2-oxo-1,2-dihydrospiro[pyrido[2,3-b][1,4]oxazine-3,3′-pyrrolidine]-1′-carboxylate (580 mg, 1.64 mmol) in DCM (12 mL) was added trifluoroacetic acid (4 mL). The mixture was stirred for 1 h at rt and concentrated under reduced pressure to afford (5'S)-6-methyl-2-oxo-1,2-dihydrospiro[pyrido[2,3-b][1,4]oxazine-3,3′-pyrrolidine]-5′-carboxamide (350 mg, crude) as a yellow oil. LC-MS (ESI, m/z): 285 [M+H]⁺.

To a stirred mixture of (5'S)-6-methyl-2-oxo-1,2-dihydrospiro[pyrido[2,3-b][1,4]oxazine-3,3′-pyrrolidine]-5′-carboxamide (290 mg, 1.10 mmol), (S)-2-((t-butoxycarbonyl)(methyl)amino)-3-cyclopropylpropanoic acid (275 mg, 1.13 mmol) and HATU (502 mg, 1.32 mmol) in DMF (6 mL) was added N-ethyl-N-isopropylpropan-2-amine (852 mg, 6.60 mmol) at 0° C. The mixture was stirred for 1 h at rt and the reaction was quenched with water (10 mL). The mixture was extracted with EA (3×30 mL). The organic layers were combined, washed with brine (2×10 mL), dried over anhydrous sodium sulfate, filtered and concentrated under reduced pressure to afford the crude product. The crude product was chromatographed on a silica gel column with MeOH:DCM (5:95) to provide tert-butyl ((2S)-1-((5'S)-5′-carbamoyl-7-methyl-3-oxo-3,4-dihydrospiro[benzo[b][1,4]oxazine-2,3′-pyrrolidin]-1′-yl)-3-cyclopropyl-1-oxopropan-2-yl)(methyl)carbamate (300 mg, 56%) as a yellow solid. ¹H NMR (400 MHz, DMSO-d₆) δ 10.52-11.47 (s, 1H), 7.32-7.67 (m, 1H), 6.87-7.30 (m, 4H), 4.58-4.49 (m, 1H), 4.32-4.57 (m, 1H), 3.73-4.17 (m, 2H), 2.84-3.04 (m, 1H), 2.35-2.47 (m, 4H), 1.35-1.47 (m, 5H), 1.02-1.32 (m, 9H), 0.52-0.68 (m, 1H), 0.32-0.41 (m, 2H), 0.01-0.03 (m, 2H). LC-MS (ESI, m/z): 487 [M+H]⁺.

To a mixture of tert-butyl ((2S)-1-((5'S)-5′-carbamoyl-6-methyl-2-oxo-1,2-dihydrospiro[pyrido[2,3-b][1,4]oxazine-3,3′-pyrrolidin]-1′-yl)-3-cyclopropyl-1-oxopropan-2-yl)(methyl)carbamate (200 mg, 0.411 mmol) in DCM (3 mL) was added trifluoroacetic acid (1 mL). The mixture was stirred for 1 h at rt and concentrated under reduced pressure to afford (5'S)-1′-((S)-3-cyclopropyl-2-(methylamino)propanoyl)-6-methyl-2-oxo-1,2-dihydrospiro[pyrido[2,3-b][1,4]oxazine-3,3′-pyrrolidine]-5′-carboxamide (159 mg, crude) as a brown semi-solid. LC-MS (ESI, m/z): 388 [M+H]⁺.

To a mixture of (5'S)-1′-((S)-3-cyclopropyl-2-(methylamino)propanoyl)-6-methyl-2-oxo-1,2-dihydrospiro[pyrido[2,3-b][1,4]oxazine-3,3′-pyrrolidine]-5′-carboxamide (159 mg, 0.410 mmol), (S)-2-((tert-butoxycarbonyl)amino)-3-cyclopropylpropanoic acid (94.0 mg, 0.410 mmol) and HATU(187 mg, 0.492 mmol) in DMF (3 mL) was added N-ethyl-N-isopropylpropan-2-amine (318 mg, 2.46 mmol) at 0° C. The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:11; Rf=0.5; detection: UV) to provide tert-butyl ((2S)-1-(((2S)-1-((5'S)-5′-carbamoyl-6-methyl-2-oxo-1,2-dihydrospiro[pyrido[2,3-b][1,4]oxazine-3,3′-pyrrolidin]-1′-yl)-3-cyclopropyl-1-oxopropan-2-yl)(methyl)amino)-3-cyclopropyl-1-oxopropan-2-yl)carbamate (185 mg, 66%) as a brown solid. ¹H NMR (400 MHz, DMSO-d₆) δ 10.94-11.09 (m, 1H), 7.33-7.49 (m, 1H), 7.15-7.22 (m, 1H), 6.90-7.11 (m, 3H), 5.24-5.34 (m, 1H), 4.30-4.43 (m, 2H), 3.99-4.15 (m, 2H), 2.88-3.01 (m, 3H), 2.64-2.76 (m, 1H), 2.28-2.37 (m, 3H), 2.02-2.22 (m, 1H), 1.42-1.68 (m, 4H), 1.29-1.37 (m, 9H), 0.55-0.70 (m, 2H), 0.20-0.45 (m, 4H), −0.02-0.10 (m, 3H), −0.31-−0.12 (m, 1H). LC-MS (ESI, m/z): 621 [M+Na]⁺.

To a mixture of tert-butyl ((2S)-1-(((2S)-1-((5'S)-5′-carbamoyl-6-methyl-2-oxo-1,2-dihydrospiro[pyrido[2,3-b][1,4]oxazine-3,3′-pyrrolidin]-1′-yl)-3-cyclopropyl-1-oxopropan-2-yl)(methyl)amino)-3-cyclopropyl-1-oxopropan-2-yl)carbamate (185 mg, 0.309 mmol) in DCM (3 mL) was added trifluoroacetic acid (1 mL). The mixture was stirred for 1 h at rt and then concentrated under reduced pressure to afford (5'S)-1′-((S)-2-((S)-2-amino-3-cyclopropyl-N-methylpropanamido)-3-cyclopropylpropanoyl)-6-methyl-2-oxo-1,2-dihydrospiro[pyrido[2,3-b][1,4]oxazine-3,3′-pyrrolidine]-5′-carboxamide (154 mg, crude) as a brown semi-solid. LC-MS (ESI, m/z): 499 [M+H]⁺.

To a mixture of (5'S)-1′-((S)-2-((S)-2-amino-3-cyclopropyl-N-methylpropanamido)-3-cyclopropylpropanoyl)-6-methyl-2-oxo-1,2-dihydrospiro[pyrido[2,3-b][1,4]oxazine-3,3′-pyrrolidine]-5′-carboxamide (154 mg, 0.309 mmol) in MeOH (3 mL) were added trimethylamine (375 mg, 3.71 mmol) and ethyl 2,2,2-trifluoroacetate (439 mg, 3.09 mmol). The mixture was stirred overnight at rt and the reaction was quenched with water (10 mL). The mixture was acidified to pH=6 with HCl (1 M) and then extracted with EA (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:11; Rf=0.4; detection: UV) to provide (5'S)-1′-((S)-3-cyclopropyl-2-((S)-3-cyclopropyl-N-methyl-2-(2,2,2-trifluoroacetamido)propanamido)propanoyl)-6-methyl-2-oxo-1,2-dihydrospiro[pyrido[2,3-b][1,4]oxazine-3,3′-pyrrolidine]-5′-carboxamide (110 mg, 56%) as a light brown solid. ¹H NMR (400 MHz, DMSO-d₆) δ 10.93-11.11 (m, 1H), 9.65-9.83 (m, 1H), 7.33-7.50 (m, 1H), 7.14-7.23 (m, 1H), 7.02-7.12 (m, 1H), 6.91-6.99 (m, 1H), 5.23-5.39 (m, 1H), 4.68-4.87 (m, 1H), 4.36-4.44 (m, 1H), 3.79-4.06 (m, 2H), 2.86-3.06 (m, 3H), 2.63-2.78 (m, 1H), 2.27-2.39 (m, 3H), 2.06-2.22 (m, 1H), 1.41-1.73 (m, 4H), 0.51-0.76 (m, 2H), 0.21-0.42 (m, 4H), −0.02-0.11 (m, 3H), −0.18-−0.09 (m, 1H). LC-MS (ESI, m/z): 617 [M+Na]⁺.

To a solution of (5'S)-1′-((S)-3-cyclopropyl-2-((S)-3-cyclopropyl-N-methyl-2-(2,2,2-trifluoroacetamido)propanamido)propanoyl)-6-methyl-2-oxo-1,2-dihydrospiro[pyrido[2,3-b][1,4]oxazine-3,3′-pyrrolidine]-5′-carboxamide (110 mg, 0.185 mmol) in DCM (3 mL) were added pyridine (73.2 mg, 0.925 mmol) and trifluoroacetic anhydride (77.8 mg, 0.370 mmol). The mixture was stirred 1 h at rt and the reaction was quenched with water (10 mL). The mixture was extracted with DCM (3×30 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:8; Rf=0.4; detection: UV) to provide the crude product. The crude product was purified by prep-ACHIRAL (Column: DAICEL DCpak P4VP 3×25 cm, Sum; Mobile Phase A: CO₂, Mobile Phase B: IPA(1%-2M-NH₃—IPA); Flow rate: 65 mL/min; Gradient: isocratic 32% B; Column Temperature(° C.): 35; Wave Length: 220 nm; RT1(min): 6.45; RT(min): 11.09; Sample Solvent: ACN; Injection Volume: 1 mL; Number Of Runs: 4) to provide (S)—N—((S)-1-((3R,5'S)-5′-cyano-6-methyl-2-oxo-1,2-dihydrospiro[pyrido[2,3-b][1,4]oxazine-3,3′-pyrrolidin]-1′-yl)-3-cyclopropyl-1-oxopropan-2-yl)-3-cyclopropyl-N-methyl-2-(2,2,2-trifluoroacetamido)propanamide (28.0 mg) that was purified twice by prep-HPLC (Column: Xselect CSH Prep C18 Column 30*150 mm, 5 m; Mobile Phase A: Water (0.1% FA), Mobile Phase B: ACN; Flow rate: 60 mL/min; Gradient: 32% B to 62% B in 10 min; Wave Length: 220 nm; RT1(min): 8.68) to provide (S)—N—((S)-1-((3R,5'S)-5′-cyano-6-methyl-2-oxo-1,2-dihydrospiro[pyrido[2,3-b][1,4]oxazine-3,3′-pyrrolidin]-1′-yl)-3-cyclopropyl-1-oxopropan-2-yl)-3-cyclopropyl-N-methyl-2-(2,2,2-trifluoroacetamido)propanamide (9.8 mg, 9%) as a white solid. ¹H NMR (400 MHz, 80° C., DMSO-d₆) δ 10.40-11.50 (br, 1H), 8.90-9.90 (br, 1H), 7.10-7.30 (m, 1H), 6.90-7.00 (m, 1H), 5.10-5.20 (m, 1H), 4.95-5.09 (m, 1H), 4.70-4.90 (m, 1H), 3.90-4.00 (m, 2H), 3.00 (s, 3H), 2.85-2.95 (m, 1H), 2.50-2.60 (m, 1H), 3.35 (s, 3H), 1.50-1.80 (m, 4H), 0.70-0.80 (m, 1H), 0.50-0.60 (m, 1H), 0.35-0.49 (m, 3H), 0.20-0.30 (m, 1H), 0.10-0.19 (m, 1H), 0.00-0.09 (m, 3H). LC-MS (ESI, m/z): 599 [M+Na]⁺.

### Example 77

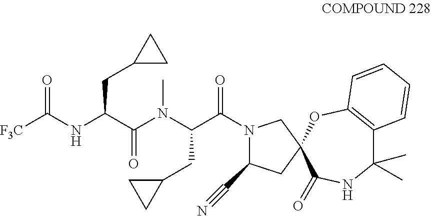 [SMILES: CN(C(=O)[C@H](CC1CC1)NC(=O)C(F)(F)F)[C@@H](CC1CC1)C(=O)N1C[C@@]2(C[C@H]1C#N)Oc1ccccc1C(C)(C)NC2=O]

Compound 228, was synthesized similarly as described for (S)—N—((S)-1-((2R,5'S)-5′-cyano-3-oxo-4,5-dihydro-3H-spiro[benzo[f][1,4]oxazepine-2,3′-pyrrolidin]-1′-yl)-3-cyclopropyl-1-oxopropan-2-yl)-3-cyclopropyl-N-methyl-2-(2,2,2-trifluoroacetamido)propenamide (Compound 81) where t-butyl (2-(2-hydroxyphenyl)propan-2-yl)carbamate was used in place of t-butyl (2-hydroxybenzyl)carbamate. The product was obtained as white solid. ¹H NMR (400 MHz, 80° C., DMSO-d₆) δ 9.27 (s, 1H), 8.05 (s, 1H), 7.37-7.43 (m, 1H), 7.30-7.36 (m, 1H), 7.20-7.27 (m, 1H), 7.01-7.12 (m, 1H), 5.23-5.37 (m, 1H), 5.00-5.11 (m, 1H), 4.77-4.99 (m, 1H), 4.16-4.28 (m, 1H), 4.03-4.12 (m, 1H), 3.09-3.15 (m, 3H), 2.64-2.75 (m, 1H), 2.52-2.60 (m, 1H), 1.65-1.76 (m, 8H), 1.55-1.64 (m, 2H), 0.56-0.76 (m, 2H), 0.28-0.47 (m, 3H), 0.17-0.27 (m, 1H), 0.08-0.16 (m, 2H), 0.00-0.07 (m, 1H), −0.09-−0.01 (m, 1H). LC-MS (ESI, m/z): 626 [M+Na]⁺.

To a mixture of 1-(2-hydroxyphenyl)ethan-1-one (20.0 g, 147 mmol) and 2-methylpropane-2-sulfinamide (35.6 g, 294 mmol) in THF (300 mL) was added dropwise titanium tetraisopropanolate (83.5 g, 294 mmol) under nitrogen. The mixture was stirred for 2 h at 65° C. under nitrogen and the reaction was quenched with water (600 mL). The mixture was extracted with EA (3×500 mL). The organic layers were combined, washed with brine (2×500 mL), dried over anhydrous sodium sulfate, filtered and concentrated under reduced pressure to afford the crude product. The crude product was chromatographed on a silica gel column with EA:PE (1:12) to provide N-(1-(2-hydroxyphenyl)ethylidene)-2-methylpropane-2-sulfinamide (7.00 g, 19%) as a light yellow oil. ¹H NMR (400 MHz, DMSO-d₆) δ 12.97 (s, 1H), 7.75-7.81 (m, 1H), 7.43-7.50 (m, 1H), 6.92-6.99 (m, 2H), 2.78 (s, 3H), 1.21 (s, 9H). LC-MS (ESI, m/z): 240 [M+H]⁺.

To a mixture of N-(1-(2-hydroxyphenyl)ethylidene)-2-methylpropane-2-sulfinamide (6.5 g, 27.1 mmol) in THF (130 mL) was added dropwise methylmagnesiumbromide (136 mL, 136 mmol, 1 M in THF) at −10° C. After stirred for 30 minutes at −10° C., the mixture was warmed to rt and stirred for 4 h. The reaction was quenched with saturated aqueous ammonium chloride (200 mL). The mixture was extracted with EA (3×200 mL). The organic layers were combined, washed with brine (2×200 mL), dried over anhydrous sodium sulfate, filtered and concentrated under reduced pressure to afford N-(2-(2-hydroxyphenyl)propan-2-yl)-2-methylpropane-2-sulfinamide (6.0 g, 77%, crude) as a yellow solid. ¹H NMR (400 MHz, DMSO-d₆) δ 9.78 (s, 1H), 7.25-7.30 (m, 1H), 7.04-7.11 (m, 1H), 6.73-6.83 (m, 2H), 5.57 (s, 1H), 1.56-1.68 (m, 6H), 1.07-1.16 (m, 9H). LC-MS (ESI, m/z): 256 [M+H]⁺.

To a mixture of N-(2-(2-hydroxyphenyl)propan-2-yl)-2-methylpropane-2-sulfinamide (5.78 g, 22.6 mmol) in dioxane (60 mL) was added HCl (17.0 mL, 2.43 mmol, 7 M in MeOH). The mixture was stirred for 1 h at rt and then concentrated under reduced pressure to afford 2-(2-aminopropan-2-yl)phenol (3.42 g, crude) as a yellow oil. LC-MS (ESI, m/z): 135 [M-NH₂+H]⁺.

To a mixture of 2-(2-aminopropan-2-yl)phenol (3.40 g, 22.5 mmol) in DCM (40 mL) was added trimethylamine (11.4 g, 112 mmol) and di-t-butyl dicarbonate (7.36 g, 33.7 mmol). The mixture was stirred for 1 h at rt and the reaction was quenched with water (100 mL). The mixture was extracted with DCM (3×100 mL). The organic layers were combined, washed with brine (2×50 mL), dried over anhydrous sodium sulfate, filtered and concentrated under reduced pressure to afford the crude product. The crude product was chromatographed on a silica gel column with EA:PE (3:97) to provide tert-butyl (2-(2-hydroxyphenyl)propan-2-yl)carbamate (2.39 g, 29%) as a yellow solid. ¹H NMR (400 MHz, DMSO-d₆) δ 9.29 (s, 1H), 7.08-7.15 (m, 1H), 6.97-7.06 (m, 1H), 6.75-6.80 (m, 1H), 6.63-6.74 (m, 2H), 1.55-1.69 (m, 6H), 1.26-1.40 (m, 9H). LC-MS (ESI, m/z): 196 [M-56+H]⁺.

### Example 78

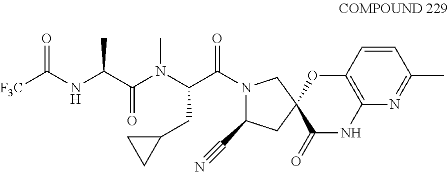 [SMILES: Cc1ccc2c(n1)NC(=O)[C@]1(C[C@@H](C#N)N(C(=O)[C@H](CC3CC3)N(C)C(=O)[C@H](C)NC(=O)C(F)(F)F)C1)O2]

To a mixture 1-(tert-butyl) 2-methyl (2S,4S)-4-hydroxy-4-(trichloromethyl)pyrrolidine-1,2-dicarboxylate (3.00 g, 8.27 mmol) and 2-bromo-6-methylpyridin-3-ol (2.64 g, 14.0 mmol) in acetone (30 mL) was added NaOH (1.99 g, 49.6 mmol) at 0° C. The mixture was stirred overnight at rt. The mixture was adjusted to pH=6 with hydrogen chloride (4 M in dioxane) and concentrated under reduced pressure to afford (2S,4R)-4-((2-bromo-6-methylpyridin-3-yl)oxy)-1-(tert-butoxycarbonyl)pyrrolidine-2,4-dicarboxylic acid (3.68 g, crude) as a light yellow solid. LC-MS (ESI, m/z): 445 [M+H]⁺.

To a mixture of (2S,4R)-4-((2-bromo-6-methylpyridin-3-yl)oxy)-1-(tert-butoxycarbonyl)pyrrolidine-2,4-dicarboxylic acid (3.68 g, 8.26 mmol) and potassium carbonate (6.85 g, 49.5 mmol) in DMF (50 mL) was added iodomethane (5.87 g, 41.3 mmol) at 0° C. The mixture was stirred for 2 h at rt. The mixture was filtered through a celite pad and the filtrate was quenched with water (150 mL). The mixture was extracted with EA (3×150 mL). The organic layers were combined, washed with brine (2×150 mL), dried over anhydrous sodium sulfate, filtered and concentrated under reduced pressure to afford the crude product. The crude product was chromatographed on a silica gel column with EA:PE (3:7) to provide 1-(tert-butyl) 2,4-dimethyl (2S,4R)-4-((2-bromo-6-methylpyridin-3-yl)oxy)pyrrolidine-1,2,4-tricarboxylate (2.30 g, 50%) as a light yellow oil. ¹H NMR (400 MHz, DMSO-d₆) δ 7.16-7.26 (m, 2H), 4.33-4.47 (m, 1H), 3.87-4.04 (m, 1H), 3.71-3.82 (m, 4H), 3.65-3.70 (m, 3H), 2.73-2.83 (m, 1H), 2.51-2.57 (m, 1H), 2.40 (s, 3H), 1.21-1.36 (m, 9H). LC-MS (ESI, m/z): 473 [M+H]⁺.

A solution of 1-(tert-butyl) 2,4-dimethyl (2S,4R)-4-((2-bromo-6-methylpyridin-3-yl)oxy)pyrrolidine-1,2,4-tricarboxylate (2.56 g, 5.43 mmol) in ammonia (90 mL, 7 M in MeOH) was stirred overnight at 50° C. The mixture was concentrated under reduced pressure to provide tert-butyl (2S,4R)-4-((2-bromo-6-methylpyridin-3-yl)oxy)-2,4-dicarbamoylpyrrolidine-1-carboxylate (2.20 g, 77%) as a yellow solid. ¹H NMR (400 MHz, DMSO-d₆) δ 8.03 (s, 1H), 7.71 (s, 1H), 7.43-7.53 (m, 1H), 7.24-7.32 (m, 1H), 7.07-7.13 (m, 1H), 6.94-7.05 (m, 2H), 4.10-4.25 (m, 1H), 3.85-3.98 (m, 1H), 3.62-3.70 (m, 1H), 2.55-2.64 (m, 1H), 2.40 (s, 3H), 2.23-2.34 (m, 1H), 1.30-1.36 (m, 5H), 1.22-1.29 (m, 4H). LC-MS (ESI, m/z): 443 [M+H]⁺.

To a mixture of tert-butyl (2S,4R)-4-((2-bromo-6-methylpyridin-3-yl)oxy)-2,4-dicarbamoylpyrrolidine-1-carboxylate (2.32 g, 5.23 mmol), cuprous iodide (596 mg, 3.14 mmol) and cesium carbonate (3.41 g, 10.4 mmol) in THF (30 mL) was added N,N′-dimethyl-1,2-ethanediamine (828 mg, 9.41 mmol). The mixture was stirred for 1 h at 70° C. under nitrogen and the reaction was quenched with water (100 mL). The mixture was extracted with EA (3×100 mL). The organic layers were combined, washed with brine (2×100 mL), dried over anhydrous sodium sulfate, filtered and concentrated under reduced pressure to afford the crude product. The crude product was chromatographed on a silica gel column with MeOH:DCM (4:96) to provide tert-butyl (2R,5'S)-5′-carbamoyl-6-methyl-3-oxo-3,4-dihydrospiro[pyrido[3,2-b][1,4]oxazine-2,3′-pyrrolidine]-1′-carboxylate (560 mg, 26%) as a light yellow solid. ¹H NMR (400 MHz, DMSO-d₆) δ 11.42 (s, 1H), 7.44-7.57 (m, 1H), 7.26-7.35 (m, 1H), 6.97-7.14 (m, 1H), 6.84-6.94 (m, 1H), 4.19-4.32 (m, 1H), 3.61-3.78 (m, 2H), 2.39-2.48 (m, 1H), 2.24-2.38 (m, 4H), 1.29-1.49 (m, 9H). LC-MS (ESI, m/z): 747 [2M+Na]⁺.

To a mixture of tert-butyl (2R,5'S)-5′-carbamoyl-6-methyl-3-oxo-3,4-dihydrospiro[pyrido[3,2-b][1,4]oxazine-2,3′-pyrrolidine]-1′-carboxylate (150 mg, 0.414 mmol) in DCM (3 mL) was added trifluoroacetic acid (1 mL) at 0° C. The mixture was stirred for 1 h at rt and then concentrated under reduced pressure to afford (2R,5'S)-6-methyl-3-oxo-3,4-dihydrospiro[pyrido[3,2-b][1,4]oxazine-2,3′-pyrrolidine]-5′-carboxamide (109 mg, crude) as a light yellow solid. LC-MS (ESI, m/z): 263 [M+H]⁺.

To a mixture of (2R,5'S)-6-methyl-3-oxo-3,4-dihydrospiro[pyrido[3,2-b][1,4]oxazine-2,3′-pyrrolidine]-5′-carboxamide (109 mg, 0.414 mmol), (S)-2-((tert-butoxycarbonyl)(methyl)amino)-3-cyclopropylpropanoic acid (101 mg, 0.414 mmol) and HATU(189 mg, 0.497 mmol) in DMF (2 mL) was added N-ethyl-N-isopropylpropan-2-amine (267 mg, 2.07 mmol) at 0° C. The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure. The residue was chromatographed on a silica gel column with MeOH:DCM (7:93) to afford tert-butyl ((S)-1-((2R,5'S)-5′-carbamoyl-6-methyl-3-oxo-3,4-dihydrospiro[pyrido[3,2-b][1,4]oxazine-2,3′-pyrrolidin]-1′-yl)-3-cyclopropyl-1-oxopropan-2-yl)(methyl)carbamate (99.0 mg, crude) as an off-white solid. LC-MS (ESI, m/z): 510 [M+Na]⁺.

To a solution of tert-butyl ((S)-1-((2R,5'S)-5′-carbamoyl-6-methyl-3-oxo-3,4-dihydrospiro[pyrido[3,2-b][1,4]oxazine-2,3′-pyrrolidin]-1′-yl)-3-cyclopropyl-1-oxopropan-2-yl)(methyl)carbamate (100 mg, 0.205 mmol) in DCM (1.5 mL) was added TFA (0.5 mL) at 0° C. The mixture was stirred for 1 h at rt and concentrated under reduced pressure to afford (2R,5'S)-1′-((S)-3-cyclopropyl-2-(methylamino)propanoyl)-6-methyl-3-oxo-3,4-dihydrospiro[pyrido[3,2-b][1,4]oxazine-2,3′-pyrrolidine]-5′-carboxamide (80.0 mg, crude) as a light yellow semi-solid. LC-MS (ESI, m/z): 388 [M+H]⁺.

To a mixture of (2R,5'S)-1′-((S)-3-cyclopropyl-2-(methylamino)propanoyl)-6-methyl-3-oxo-3,4-dihydrospiro[pyrido[3,2-b][1,4]oxazine-2,3′-pyrrolidine]-5′-carboxamide (80.0 mg, 0.206 mmol), (t-butoxycarbonyl)-L-alanine (39.0 mg, 0.206 mmol) and HATU (95.0 mg, 0.247 mmol) in DMF (2 mL) was added N-ethyl-N-isopropylpropan-2-amine (160 mg, 1.2 mmol) at 0° C. The mixture was stirred for 1 h at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:11; Rf=0.4; detection: UV) to provide tert-butyl ((S)-1-(((S)-1-((2R,5'S)-5′-carbamoyl-6-methyl-3-oxo-3,4-dihydrospiro[pyrido[3,2-b][1,4]oxazine-2,3′-pyrrolidin]-1′-yl)-3-cyclopropyl-1-oxopropan-2-yl)(methyl)amino)-1-oxopropan-2-yl)carbamate (75.0 mg, 56%) as an off-white solid. ¹H NMR (400 MHz, DMSO-d₆) δ 11.48 (s, 1H), 7.45 (s, 1H), 7.26-7.30 (m, 1H), 6.99-7.03 (s, 1H), 6.92-6.98 (m, 1H), 6.85-6.89 (m, 1H), 5.14-5.21 (m, 1H), 4.33-4.39 (m, 1H), 4.24-4.30 (m, 1H), 3.83-3.90 (m, 2H), 2.90 (s, 3H), 2.41-2.48 (m, 1H), 2.35 (s, 3H), 2.20-2.27 (m, 1H), 1.44-1.53 (m, 2H), 1.30-1.35 (m, 9H), 0.81-0.86 (m, 3H), 0.56-0.66 (m, 1H), 0.22-0.39 (m, 2H), 0.01-0.10 (m, 2H). LC-MS (ESI, m/z): 581 [M+Na]⁺.

To a mixture of tert-butyl ((S)-1-(((S)-1-((2R,5'S)-5′-carbamoyl-6-methyl-3-oxo-3,4-dihydrospiro[pyrido[3,2-b][1,4]oxazine-2,3′-pyrrolidin]-1′-yl)-3-cyclopropyl-1-oxopropan-2-yl)(methyl)amino)-1-oxopropan-2-yl)carbamate (75.0 mg, 0.159 mmol) in DCM (1.5 mL) was added TFA (0.50 mL) at 0° C. The mixture was stirred for 1 h at rt and concentrated under reduced pressure to afford (2R,5'S)-1′-((S)-2-((S)-2-amino-N-methylpropanamido)-3-cyclopropylpropanoyl)-6-methyl-3-oxo-3,4-dihydrospiro[pyrido[3,2-b][1,4]oxazine-2,3′-pyrrolidine]-5′-carboxamide (73.0 mg, crude) as an light brown semi-solid. LC-MS (ESI, m/z): 459 [M+H]⁺.

To a mixture of (2R,5'S)-1′-((S)-2-((S)-2-amino-N-methylpropanamido)-3-cyclopropylpropanoyl)-6-methyl-3-oxo-3,4-dihydrospiro[pyrido[3,2-b][1,4]oxazine-2,3′-pyrrolidine]-5′-carboxamide (73.0 mg, 0.159 mmol) in MeOH (1 mL) was added triethylamine (193 mg, 1.90 mmol) and ethyl 2,2,2-trifluoroacetate (226 mg, 1.59 mmol). The mixture was stirred overnight at rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:11; Rf=0.4; detection: UV) to provide (2R,5'S)-1′-((S)-3-cyclopropyl-2-((S)—N-methyl-2-(2,2,2-trifluoroacetamido)propanamido)propanoyl)-6-methyl-3-oxo-3,4-dihydrospiro[pyrido[3,2-b][1,4]oxazine-2,3′-pyrrolidine]-5′-carboxamide (45.0 mg, 39%) as an off-white solid. ¹H NMR (400 MHz, DMSO-d₆) δ 11.44-11.52 (m, 1H), 9.57-9.65 (m, 1H), 7.45 (s, 1H), 7.27-7.33 (m, 1H), 7.0 (s, 1H), 6.84-6.90 (m, 1H), 5.13-5.20 (m, 1H), 4.62-4.69 (m, 1H), 4.34-4.40 (m, 1H), 3.87-3.96 (m, 2H), 2.92 (s, 3H), 2.42-2.48 (m, 1H), 2.35 (s, 3H), 2.18-2.25 (m, 1H), 1.57-1.68 (m, 1H), 1.39-1.49 (m, 1H), 1.00-1.07 (m, 3H), 0.53-0.62 (m, 1H), 0.24-0.38 (m, 2H), 0.00-0.08 (m, 2H). LC-MS (ESI, m/z): 577 [M+Na]⁺.

To a mixture of (2R,5'S)-1′-((S)-3-cyclopropyl-2-((S)—N-methyl-2-(2,2,2-trifluoroacetamido)propanamido)propanoyl)-6-methyl-3-oxo-3,4-dihydrospiro[pyrido[3,2-b][1,4]oxazine-2,3′-pyrrolidine]-5′-carboxamide (45.0 mg, 0.081 mmol) in DCM (1 mL) was added pyridine (32.0 mg, 0.405 mmol) and trifluoroacetic anhydride (34.0 mg, 0.162 mmol). The mixture was stirred for 1 h at rt and the reaction was quenched with water (5 mL). The mixture was extracted with DCM (3×5 mL). The organic layers were combined, washed with brine (2×5 mL), dried over anhydrous sodium sulfate, filtered and concentrated under reduced pressure to afford the crude product. The crude product was purified by prep-HPLC (Column: XselectCSH Prep OBD C18 Column, 19×250 mm, 5 m; Mobile Phase A: Water (0.1% FA), Mobile Phase B: ACN; Flow rate: 25 mL/min; Gradient: 40% B to 55% B in 8 min; Wave Length: 220 nm; RT1(min): 6.78) to provide (S)—N—((S)-1-((2R,5'S)-5′-cyano-6-methyl-3-oxo-3,4-dihydrospiro[pyrido[3,2-b][1,4]oxazine-2,3′-pyrrolidin]-1′-yl)-3-cyclopropyl-1-oxopropan-2-yl)-N-methyl-2-(2,2,2-trifluoroacetamido)propanamide (18.3 mg, 42%) as a white solid. ¹H NMR (400 MHz, 80° C., DMSO-d₆) δ 11.27 (br, 1H), 9.32 (br, 1H), 7.15-7.30 (m, 1H), 6.75-6.90 (m, 1H), 5.04-5.22 (m, 1H), 4.80-5.03 (m, 1H), 4.58-4.78 (m, 1H), 3.90-4.15 (m, 2H), 2.96 (s, 3H), 2.60-2.85 (m, 2H), 2.35 (s, 3H), 1.50-1.70 (m, 2H), 0.98-1.25 (m, 3H), 0.50-0.70 (m, 1H), 0.20-0.45 (m, 2H), 0.00-0.15 (m, 2H). LC-MS (ESI, m/z): 559 [M+Na]⁺.

### Example 79

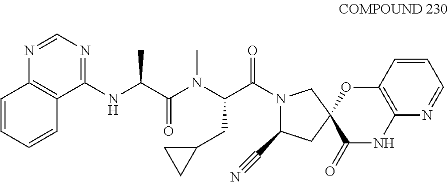 [SMILES: C[C@H](Nc1ncnc2ccccc12)C(=O)N(C)[C@@H](CC1CC1)C(=O)N1C[C@@]2(C[C@H]1C#N)Oc1cccnc1NC2=O]

A mixture of (2R,5'S)-1′-((S)-2-((S)-2-amino-N-methylpropanamido)-3-cyclopropylpropanoyl)-3-oxo-3,4-dihydrospiro[pyrido[3,2-b][1,4]oxazine-2,3′-pyrrolidine]-5′-carboxamide (90.0 mg, 0.202 mmol), 4-chloroquinazoline (67.0 mg, 0.404 mmol), triethylamine (102 mg, 1.01 mmol) and cesium fluoride (62.0 mg, 0.404 mmol) in DMSO (4 mL) was stirred for 2 h at rt. The mixture was purified by C18 column with CH₃CN:Water (0.05% TFA). The fraction was concentrated under reduced pressure to afford (2R,5'S)-1′-((S)-3-cyclopropyl-2-((S)—N-methyl-2-(quinazolin-4-ylamino)propanamido)propanoyl)-3-oxo-3,4-dihydrospiro[pyrido[3,2-b][1,4]oxazine-2,3′-pyrrolidine]-5′-carboxamide (110 mg, crude) as an off-white solid. LC-MS (ESI, m/z): 573 [M+H]⁺.

To a mixture of (2R,5'S)-1′-((S)-3-cyclopropyl-2-((S)—N-methyl-2-(quinazolin-4-ylamino)propanamido)propanoyl)-3-oxo-3,4-dihydrospiro[pyrido[3,2-b][1,4]oxazine-2,3′-pyrrolidine]-5′-carboxamide (100 mg, 0.175 mmol) in DCM (10 mL) was added burgess reagent (499 mg, 2.10 mmol). The mixture was stirred for 1 h at rt and the reaction was quenched with water (20 mL). The mixture was extracted with DCM (3×25 mL). The organic layers were combined, washed with brine (2×25 mL), dried over anhydrous sodium sulfate, filtered and concentrated under reduced pressure. The crude product was purified by TLC (Mobile phase: MeOH:DCM=1:10; Rf=0.4; detection: UV), then by prep-HPLC (Column: Xselect CSH Prep C18 Column, 30×150 mm, 5 m; Mobile Phase A: Water (0.1% FA), Mobile Phase B: ACN; Flow rate: 60 mL/min; Gradient: 20 to 50% B in 10 min; Wave Length: 220 nm; RT(min): 8.5) to provide (S)—N—((S)-1-((2R,5'S)-5′-cyano-3-oxo-3,4-dihydrospiro[pyrido[3,2-b][1,4]oxazine-2,3′-pyrrolidin]-1′-yl)-3-cyclopropyl-1-oxopropan-2-yl)-N-methyl-2-(quinazolin-4-ylamino)propanamide (8.4 mg, 8%) as a white solid. ¹H NMR (400 MHz, 80° C., DMSO-d₆) δ 11.40 (br, 1H), 8.34-8.42 (m, 2H), 7.95-8.07 (m, 2H), 7.73-7.82 (m, 1H), 7.65-7.72 (m, 1H), 7.47-7.56 (m, 1H), 7.34-7.43 (m, 1H), 6.96-7.07 (m, 1H), 5.09-5.30 (m, 2H), 4.95-5.08 (m, 1H), 4.01-4.20 (m, 2H), 2.90-3.03 (m, 3H), 2.77-2.88 (m, 1H), 2.64-2.76 (m, 1H), 1.62-1.74 (m, 2H), 1.22-1.37 (m, 3H), 0.63-0.74 (m, 1H), 0.34-0.45 (m, 1H), 0.18-0.31 (m, 1H), 0.04-0.30 (in, 2H). LC-MS (ESI, m/z): 555 [M+H]⁺.

The following compounds were made using analogous methods as for those described above.

| | | LC-MS | |
|---|---|---|---|
| Entry | Structure | (ESI, m/z) | NMR |
| 231 | 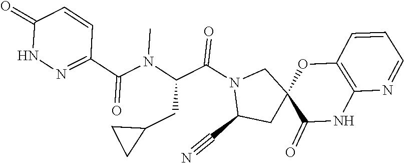 [SMILES: CN(C(=O)c1ccc(=O)[nH]n1)[C@@H](CC1CC1)C(=O)N1C[C@@]2(C[C@H]1C#N)Oc1cccnc1NC2=O] | 500 [M + Na]⁺ | ¹H NMR (400 MHz, 80º C., DMSO-d₆) δ 7.93-7.99 (m, 1H), 7.33-7.40 (m, 1H), 7.28-7.32 (m, 1H), 6.95-6.99 (m, 1H), 6.84- 6.94 (m, 1H), 5.14-5.30 (m, 1H), 4.92-5.13 (m, 1H), 4.10-4.30 (m, 1H), 3.88-4.09 (m, 1H), 3.00- 3.05 (m, 3H), 2.75-2.85 (m, 2H), 1.65-1.85 (m, 2H), 0.50-0.80 (m, 1H), 0.27-0.49 (m, 2H), 0.00- 0.26 (m, 2H). |
| 232 | 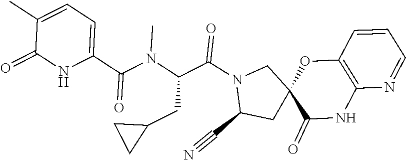 [SMILES: Cc1ccc(C(=O)N(C)[C@@H](CC2CC2)C(=O)N2C[C@@]3(C[C@H]2C#N)Oc2cccnc2NC3=O)[nH]c1=O] | 491 [M + H]⁺ | ¹H NMR (400 MHz, 80º C., DMSO-d6) δ 11.12 (br, 1H), 7.78-7.87 (m, 1H), 7.11-7.26 (m, 2H), 6.76-6.89 (m, 1H), 6.13 (s, 1H), 4.76-5.09 (m, 2H), 3.80- 4.12 (m, 2H), 2.84 (s, 3H), 2.54- 2.78 (m, 2H), 1.91 (s, 3H), 1.53- 1.69 (m, 2H), 0.52-0.66 (m, 1H), 0.23-0.36 (m, 2H), −0.07-0.05 (m, 2H). |
| 233 | 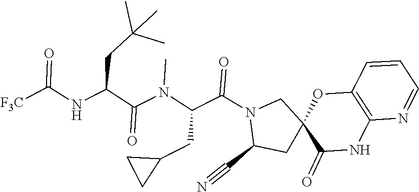 [SMILES: CN(C(=O)[C@H](CC(C)(C)C)NC(=O)C(F)(F)F)[C@@H](CC1CC1)C(=O)N1C[C@@]2(C[C@H]1C#N)Oc1cccnc1NC2=O] | 579 [M + H]⁺ | ¹H NMR (500 MHz, 363K, DMSO-d6) δ 11.30 (br. s., 1H), 9.30 (br. s., 1H), 7.94 (m, 1H), 7.23 (m, 1H), 6.96 (m, 1H), 5.10 (m, 1H), 4.97 (m, 1H), 4.80 (m, 1H), 4.10 (s, 2H), 3.04 (s, 3H), 2.59-2.78 (m, 2H), 1.68-1.81 (m, 2H), 1.64 (m, 1H), 1.54-1.60 (m, 1H), 0.89 (s, 9H), 0.63 (m, 1H), 0.34-0.46 (m, 2H), 0.11 (m, 2H) |
| 234 | 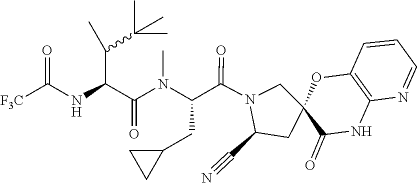 [SMILES: CC([C@H](NC(=O)C(F)(F)F)C(=O)N(C)[C@@H](CC1CC1)C(=O)N1C[C@@]2(C[C@H]1C#N)Oc1cccnc1NC2=O)C(C)(C)C] | 363⁺ (fragment peak). | ¹H NMR (400 MHz, 80° C., DMSO-d₆) δ 11.35 (br, 1H), 8.70 (s, 1H), 7.85-8.00 (m, 1H), 7.15-7.30 (m, 1H), 6.82-6.95 (m, 1H), 5.06-5.31 (m, 1H), 4.95- 5.05 (m, 1H), 4.78-4.94 (m, 1H), 4.02-4.20 (m, 1H), 3.88-4.01 (m, 1H), 2.88-2.95 (m, 3H), 2.60- 2.73 (m, 1H), 2.40-2.55 (m, 1H), 1.58-1.75 (m, 2H), 1.40-1.57 (m, 1H), 0.78-0.82 (m, 1H), 0.70- |
| | | | 0.77 (m, 3H), 0.60-0.69 (m, 8H), |
| | | | 0.45-0.59 (m, 1H), 0.15-0.35 (m, |
| | | | 2H), −0.05-0.10 (m, 2H) |
| 235 | 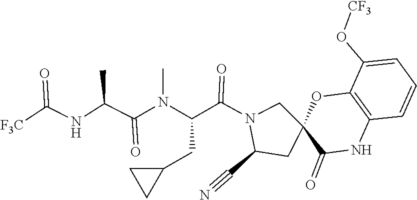 [SMILES: C[C@H](NC(=O)C(F)(F)F)C(=O)N(C)[C@@H](CC1CC1)C(=O)N1C[C@@]2(C[C@H]1C#N)Oc1c(cccc1OC(F)(F)F)NC2=O] | 628 [M + Na]⁺ | ¹H NMR (400 MHz, 80° C., DMSO-d₆) δ 11.14 (br, 1H), 9.32 (br, 1H), 7.06-7.13 (m, 1H), 6.92- 7.05 (m, 2H), 5.16-5.35 (m, 1H), 4.77-4.91 (m, 1H), 4.63-4.76 (m, 1H), 4.06-4.26 (m, 1H), 3.90- 4.05 (m, 1H), 2.94 (s, 3H), 2.74- 2.83 (m, 1H), 2.59-2.73 (m, 1H), 1.53-1.76 (m, 2H), 1.06-1.40 (m, 3H), 0.51-0.68 (m, 1H), 0.28- 0.42 (m, 2H), 0.01-0.17 (m, 2H). |
| 236 | 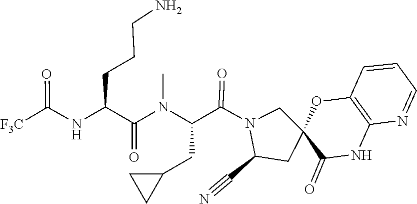 [SMILES: CN(C(=O)[C@H](CCCN)NC(=O)C(F)(F)F)[C@@H](CC1CC1)C(=O)N1C[C@@]2(C[C@H]1C#N)Oc1cccnc1NC2=O] | 566 [M + H]⁺ | ¹H NMR (400 MHz, 80° C., DMSO-d₆) δ 7.90-8.01 (m, 1H), 7.25-7.40 (m, 1H), 6.89-7.06 (m, 1H), 5.09-5.29 (m, 1H), 4.86- 5.07 (m, 1H), 4.56-4.78 (m, 1H), 3.99-4.18 (m, 2H), 3.00 (s, 3H), 2.62-2.81 (m, 3H), 2.44-2.48 (m, 1H), 1.52-1.71 (m, 4H), 1.20- 1.40 (m, 2H), 0.54-0.69 (m, 1H), 0.30-0.44 (m, 2H), 0.04-0.16 (m, 2H). |
| 237 | 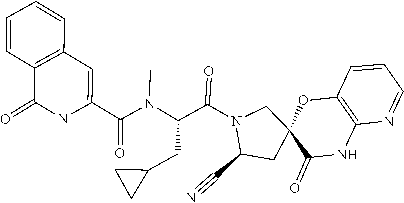 | 527 [M + H]⁺ | ¹H NMR (400 MHZ, 80° C., DMSO-d₆) δ 10.95-11.75 (m, 2H), 8.12-8.25 (m, 1H), 7.90- 8.00 (m, 1H), 7.66-7.75 (m, 1H), 7.55-7.65 (m, 1H), 7.50-7.54 (m, 1H), 7.30-7.45 (m, 1H), 6.85- 6.95 (m, 1H), 6.42-6.60 (m, 1H), 4.90-5.20 (m, 2H), 4.00-4.30 (m, 2H), 3.00-3.05 (m, 3H), 2.70- 2.90 (m, 2H), 1.65-1.90 (m, 2H), 0.70-0.86 (m, 1H), 0.35-0.55 (m, 2H), 0.10-0.20 (m, 2H). |
| 238 | 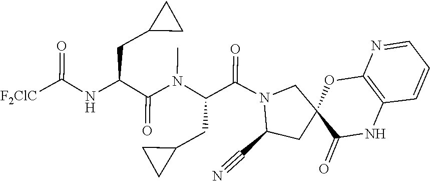 | 579 [M + H]⁺ | ¹H NMR (400 MHz, 80° C., DMSO-d₆) δ 11.05 (br, 1H), 9.11 (br, 1H), 7.81-7.87 (m, 1H), 7.25- 7.35 (m, 1H), 7.00-7.11 (m, 1H), 5.20-5.40 (m, 1H), 4.85-5.15 (m, 1H), 4.60-4.80 (m, 1H), 4.00- 4.25 (m, 2H), 2.90 (s, 3H), 2.78- 2.90 (m, 1H), 2.60-2.77 (m, 1H), 1.60-1.75 (m, 2H), 1.30-1.59 (m, 2H), 0.50-0.70 (m, 2H), 0.20- 0.45 (m, 4H), 0.01-0.15 (m, 3H), −0.12-0.00 (m, 1H). |
| 239 | 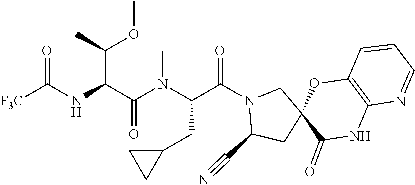 [SMILES: CO[C@H](C)[C@H](NC(=O)C(F)(F)F)C(=O)N(C)[C@@H](CC1CC1)C(=O)N1C[C@@]2(C[C@H]1C#N)Oc1cccnc1NC2=O] | 589 [M + Na]⁺ | ¹H NMR (400 MHz, 80° C., DMSO-d₆) δ 11.37 (br, 1H), 9.22 (br, 1H), 7.90-8.00 (m, 1H), 7.26- 7.35 (m, 1H), 6.90-7.10 (m, 1H), 5.02-5.25 (m, 1H), 4.80-5.01 (m, 1H), 4.60-4.78 (m, 1H), 3.96- 4.25 (m, 2H), 3.55-3.79 (m, 1H), 3.21 (s, 3H), 3.02-3.06 (m, 3H), 2.75-2.85 (m, 1H), 2.60-2.74 (m, 1H), 1.55-1.75 (m, 2H), 0.94- 1.06 (m, 3H), 0.51-0.73 (m, 1H), |
| | | | 0.25-0.50 (m, 2H), 0.00-0.15 (m, |
| | | | 2H). |
| 240 | 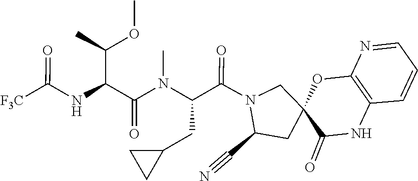 [SMILES: CO[C@H](C)[C@H](NC(=O)C(F)(F)F)C(=O)N(C)[C@@H](CC1CC1)C(=O)N1C[C@@]2(C[C@H]1C#N)Oc1ncccc1NC2=O] | 567 [M + H]⁺ | ¹H NMR (400 MHz, 80° C., DMSO-d₆) δ 10.61-11.30 (br, 1H), 8.90-9.50 (br, 1H), 7.80- 7.90 (m, 1H), 7.20-7.35 (m, 1H), 7.00-7.10 (m, 1H), 5.15-5.32 (m, 1H), 4.90-5.10 (m, 1H), 4.60- 4.72 (m, 1H), 4.10-4.23 (m, 2H), 3.60-3.75 (m, 1H), 3.22 (s, 3H), 2.90-3.00 (m, 3H), 2.80-2.88 (m, 1H), 2.60-2.78 (m, 1H), 1.55- 1.80 (m, 2H), 0.90-1.10 (m, 3H), |
| | | | 0.55-0.70 (m, 1H), 0.30-0.45 (m, |
| | | | 2H), 0.01-0.15 (m, 2H). |
| 241 | 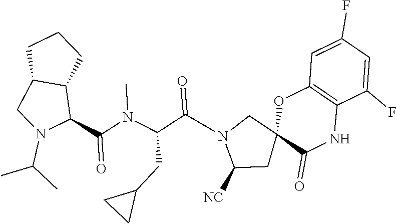 [SMILES: CC(C)N1C[C@@H]2CCC[C@@H]2[C@H]1C(=O)N(C)[C@@H](CC1CC1)C(=O)N1C[C@@]2(C[C@H]1C#N)Oc1cc(F)cc(F)c1NC2=O] | 570 [M + H]⁺ | ¹H NMR (500 MHz, 363K, DMSO-d₆) δ 11.00 (br. s., 1H), 6.94 (m, 1H), 6.67 (m, 1H), 5.18 (m, 1H), 4.94 (m, 1H), 4.03-4.27 (m, 2H), 3.49 (s, 1H), 3.21 (m, 1H), 3.04 (s, 3H), 2.80 (m, 2H), 2.67 (m, 1H), 2.38-2.46 (m, 1H), 2.32 (m, 2H), 1.53-1.76 (m, 5H), 1.29-1.51 (m, 3H), 0.94 (d, 3H), 0.87 (d, 3H), 0.62 (m, 1H), 0.38 (m, 2H), 0.11 (m, 2H) |
| 242 | 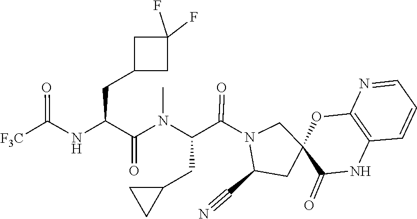 [SMILES: CN(C(=O)[C@H](CC1CC(F)(F)C1)NC(=O)C(F)(F)F)[C@@H](CC1CC1)C(=O)N1C[C@@]2(C[C@H]1C#N)Oc1ncccc1NC2=O] | 613 [M + H]⁺ | ¹H NMR (400 MHz, 80° C., DMSO-d₆) δ 9.32 (br, 1H), 7.70- 7.84 (m, 1H), 7.17-7.38 (m, 1H), 6.81-7.16 (m, 1H), 5.07-5.31 (m, 1H), 4.79-4.98 (m, 1H), 4.46- 4.66 (m, 1H), 3.93-4.20 (m, 2H), 2.91 (s, 3H), 2.71-2.81 (m, 1H), 2.55-2.66 (m, 1H), 2.45-2.53 (m, 2H), 1.97-2.21 (m, 3H), 1.67- 1.84 (m, 2H), 1.49-1.66 (m, 2H), 0.41-0.61 (m, 1H), 0.19-0.40 (m, 2H), −0.06-0.10 (m, 2H). |
| 243 | 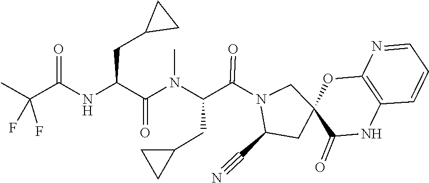 [SMILES: CN(C(=O)[C@H](CC1CC1)NC(=O)C(C)(F)F)[C@@H](CC1CC1)C(=O)N1C[C@@]2(C[C@H]1C#N)Oc1ncccc1NC2=O] | 559 [M + H]⁺ | ¹H NMR (400 MHz, 80° C., DMSO-d₆) δ 11.19 (br, 1H), 8.15-8.32 (m, 1H), 7.70-7.80 (m, 1H), 7.18-7.30 (m, 1H), 6.90- 7.05 (m, 1H), 5.05-5.23 (m, 1H), 4.78-4.98 (m, 1H), 4.50-4.77 (m, 1H), 3.90-4.15 (m, 2H), 2.85- 2.95 (m, 3H), 2.69-2.80 (m, 1H), 2.50-2.68 (m, 1H), 1.49-1.70 (m, 5H), 1.25-1.48 (m, 2H), 0.45- 0.65 (m, 2H), 0.13-0.40 (m, 4H), |
| | | | −0.05-0.15 (m, 2H), −0.25-−0.04 |
| | | | (m, 2H). |
| 244 | 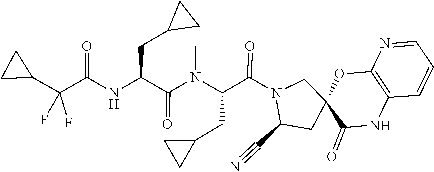 [SMILES: CN(C(=O)[C@H](CC1CC1)NC(=O)C(F)(F)C1CC1)[C@@H](CC1CC1)C(=O)N1C[C@@]2(C[C@H]1C#N)Oc1ncccc1NC2=O] | 585 [M + H]⁺ | ¹H NMR (400 MHz, 80° C., DMSO-d₆) δ 10.98 (br, 1H), 8.10-8.35 (m, 1H), 7.65-7.85 (m, 1H), 7.15-7.30 (m, 1H), 6.90- 7.10 (m, 1H), 5.00-5.30 (m, 1H), 4.65-5.00 (m, 1H), 4.50-4.64 (m, 1H), 3.90-4.10 (m, 2H), 2.80- 2.86 (m, 3H), 2.69-2.79 (m, 1H), 2.51-2.68 (m, 1H), 1.50-1.75 (m, 2H), 1.40-1.49 (m, 2H), 1.20- 1.39 (m, 1H), 0.40-0.60 (m, 6H), 0.15-0.34 (m, 4H), −0.08-0.10 (m, 2H), −0.30-−0.07 (m, 2H). |
| 245 | 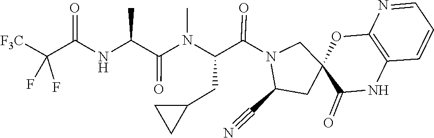 [SMILES: C[C@H](NC(=O)C(F)(F)C(F)(F)F)C(=O)N(C)[C@@H](CC1CC1)C(=O)N1C[C@@]2(C[C@H]1C#N)Oc1ncccc1NC2=O] | 573 [M + H]⁺ | ¹H NMR (400 MHz, 80° C., DMSO-d₆) δ 10.98 (s, 1H), 9.29 (s, 1H), 7.65-7.85 (m, 1H), 7.20- 7.30 (m, 1H), 6.90-7.10 (m, 1H), 5.00-5.29 (m, 1H), 4.80-4.99 (m, 1H), 4.50-4.75 (m, 1H), 3.80- 4.15 (m, 2H), 2.87 (s, 3H), 2.70- 2.81 (m, 1H), 2.61-2.69 (m, 1H), 1.45-1.69 (m, 2H), 0.80-1.20 (m, |
| | | | 3H), 0.40-0.59 (m, 1H), 0.15- |
| | | | 0.39 (m, 2H), −0.10-0.10 (m, |
| | | | 2H). |
| 246 | 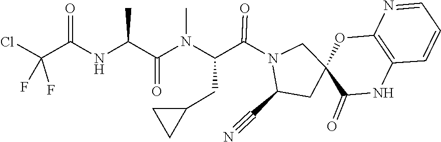 [SMILES: C[C@H](NC(=O)C(F)(F)Cl)C(=O)N(C)[C@@H](CC1CC1)C(=O)N1C[C@@]2(C[C@H]1C#N)Oc1ncccc1NC2=O] | 539 [M + H]⁺ | ¹H NMR (400 MHz, 80° C., DMSO-d₆) δ 9.11 (br, 1H), 7.65- 7.80 (m, 1H), 7.15-7.30 (m, 1H), 6.90-7.10 (m, 1H), 5.03-5.23 (m, 1H), 4.78-5.02 (m, 1H), 4.40- 4.65 (m, 1H), 3.75-4.15 (m, 2H), 2.88 (s, 3H), 2.72-2.80 (m, 1H), 2.56-2.71 (m, 1H), 1.40-1.70 (m, 2H), 0.80-1.15 (m, 3H), 0.45- |
| | | | 0.65 (m, 1H), 0.15-0.40 (m, 2H), |
| | | | −0.08-0.12 (m, 2H). |
| 247 | 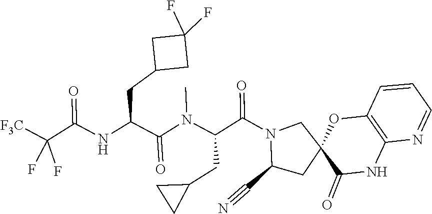 [SMILES: CN(C(=O)[C@H](CC1CC(F)(F)C1)NC(=O)C(F)(F)C(F)(F)F)[C@@H](CC1CC1)C(=O)N1C[C@@]2(C[C@H]1C#N)Oc1cccnc1NC2=O] | 685 [M + Na]⁺ | ¹H NMR (400 MHz, 80 ° C., DMSO-d₆) δ 11.32 (br, 1H), 9.40 (br, 1H), 7.81-8.00 (m, 1H), 7.22- 7.39 (m, 1H), 6.80-7.00 (m, 1H), 4.99-5.20 (m, 1H), 4.81-4.98 (m, 1H), 4.49-4.70 (m, 1H), 3.93- 4.15 (m, 2H), 2.92 (s, 3H), 2.68- 2.79 (m, 1H), 2.53-2.67 (m, 1H), 2.41-2.51 (m, 2H), 1.96-2.20 (m, 3H), 1.67-1.90 (m, 2H), 1.50- 1.66 (m, 2H), 0.42-0.61 (m, 1H), 0.17-0.40 (m, 2H), −0.06-0.10 (m, 2H). |
| 248 | 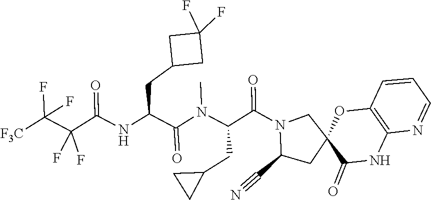 [SMILES: CN(C(=O)[C@H](CC1CC(F)(F)C1)NC(=O)C(F)(F)C(F)(F)C(F)(F)F)[C@@H](CC1CC1)C(=O)N1C[C@@]2(C[C@H]1C#N)Oc1cccnc1NC2=O] | 645 [M + Na]⁺ | ¹H NMR (400 MHz, 80° C., DMSO-d₆) δ 11.40 (br, 1H), 9.20-9.57 (m, 1H), 7.87-8.10 (m, 1H), 7.25-7.42 (m, 1H), 6.89- 7.09 (m, 1H), 5.04-5.25 (m, 1H), 4.86-5.03 (m, 1H), 4.58-4.82 (m, 1H), 3.91-4.10 (m, 2H), 2.96 (s, 3H), 2.75-2.84 (m, 1H), 2.68- 2.74 (m, 1H), 1.51-1.74 (m, 2H), 1.01-1.21 (m, 3H), 0.51-0.69 (m, 1H), 0.26-0.44 (m, 2H), 0.02- 0.15 (m, 2H). |
| 249 | 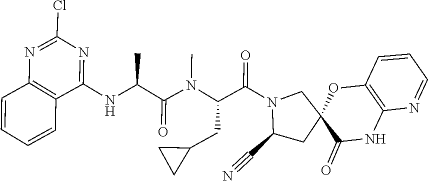 [SMILES: C[C@H](Nc1nc(Cl)nc2ccccc12)C(=O)N(C)[C@@H](CC1CC1)C(=O)N1C[C@@]2(C[C@H]1C#N)Oc1cccnc1NC2=O] | 589 [M + H]⁺ | ¹H NMR (400 MHz, 80º C., DMSO-d₆) δ 11.40 (br, 1H), 8.50 (s, 1H), 8.28-8.40 (m, 1H), 7.85- 8.00 (m, 1H), 7.65-7.80 (m, 1H), 7.50-7.60 (m, 1H), 7.40-7.49 (m, 1H), 7.20-7.38 (m, 1H), 6.80- 7.00 (m, 1H), 5.05-5.22 (m, 1H), 4.80-5.04 (m, 2H), 3.85-4.10 (m, 2H), 3.15-3.22 (m, 3H), 2.70- 2.85 (m, 1H), 2.58-2.69 (m, 1H), 1.45-1.75 (m, 2H), 1.10-1.30 (m, |
| | | | 3H), 0.50-0.70 (m, 1H), 0.25- |
| | | | 0.35 (m, 1H), 0.10-0.24 (m, 1H), |
| | | | −0.10-0.09 (m, 2H). |

### (2S)-2-((tert-butoxycarbonyl)amino)-3,4,4-trimethylpentanoic acid

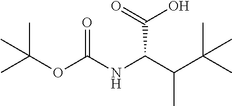 [SMILES: CC([C@H](NC(=O)OC(C)(C)C)C(=O)O)C(C)(C)C]

To a mixture of (S)-2,4,6-trimethylbenzenesulfinamide (400 mg, 2.18 mmol) and magnesium sulfate (1.31 g, 10.9 mmol) in DCM (12 mL) were added pyrrolidine (16.0 mg, 0.218 mmol) and ethyl glyoxylate (1.34 g, 6.55 mmol, 50% in toluene). The mixture was stirred overnight at rt. The mixture was filtered and the filtrate was concentrated under reduced pressure to afford ethyl 2-{[(S)-2,4,6-trimethylbenzenesulfinyl]imino}acetate (584 mg, crude) as a light yellow oil. LC-MS (ESI, m/z): 268 [M+H]⁺.

To a mixture of 2,3,3-trimethylbutanoic acid (300 mg, 2.30 mmol), 4,5,6,7-tetrachloro-2-hydroxyisoindoline-1,3-dione (763 mg, 2.53 mmol) and N,N-dimethylpyridin-4-amine (56.0 mg, 0.461 mmol) in DCM (9 mL) was added N,N′-diisopropylcarbodiimide (320 mg, 2.53 mmol). The mixture was stirred for 1 h at rt. The mixture was chromatographed on a silica gel column with EA:PE (9:91) to provide 4,5,6,7-tetrachloro-1,3-dioxoisoindol-2-yl 2,3,3-trimethylbutanoate (750 mg, 74.85%) as an off-white solid. ¹H NMR (400 MHz, DMSO-d₆) δ 2.76 (q, J=7.2 Hz, 1H), 1.21 (d, J=7.2 Hz, 3H), 1.04 (s, 9H).

A mixture of 4,5,6,7-tetrachloro-1,3-dioxoisoindolin-2-yl 2,3,3-trimethylbutanoate (600 mg, 1.45 mmol), ethyl 2-{[(S)-2,4,6-trimethylbenzenesulfinyl]imino}acetate (582 mg, 2.18 mmol), zinc (285 mg, 4.36 mmol) and nickel(II) acetate tetrahydrate (90.0 mg, 0.363 mmol) in NMP (12 mL) was stirred overnight at rt under nitrogen and the reaction was quenched with water (50 mL). The mixture was extracted with EA (3×50 mL). The organic layers were combined, washed with brine (2×50 mL), dried over anhydrous sodium sulfate, filtered and concentrated under reduced pressure. The crude product was purified by TLC (Mobile phase: EA:PE=1:5; Rf=0.5; detection: UV) to provide ethyl (2S)-2-(((S)-mesitylsulfinyl)amino)-3,4,4-trimethylpentanoate (120 mg, 21%) as a yellow oil. ¹H NMR (400 MHz, Chloroform-d) δ 6.85-6.90 (m, 2H), 6.07-6.57 (m, 1H), 3.81-4.15 (m, 3H), 2.50-2.55 (m, 6H), 2.23 (s, 3H), 1.52-1.71 (m, 1H), 1.03-1.11 (m, 3H), 0.88-0.98 (m, 9H), 0.76-0.86 (m, 3H). LC-MS (ESI, m/z): 354 [M+H]⁺.

To a mixture of ethyl (2S)-2-(((S)-mesitylsulfinyl)amino)-3,4,4-trimethylpentanoate (120 mg, 0.339 mmol) in MeOH (1.7 mL) was added hydrogen chloride (0.34 mL, 1.36 mmol, 4 M in dioxane) at 0° C. The mixture was stirred for 1 h at rt. The mixture was concentrated under reduced pressure to afford ethyl (2S)-2-amino-3,4,4-trimethylpentanoate (64 mg, crude) as a brown oil. LC-MS (ESI, m/z): 188 [M+H]⁺.

To a mixture of ethyl (2S)-2-amino-3,4,4-trimethylpentanoate (64.0 mg, 0.342 mmol) and di-t-butyl dicarbonate (112 mg, 0.513 mmol) in DCM (2 mL) was added triethylamine (173 mg, 1.71 mmol). The mixture was stirred for 2 h at rt and the reaction was quenched with water (5 mL). The mixture was extracted with DCM (3×5 mL). The organic layers were combined, washed with brine (2×5 mL), dried over anhydrous sodium sulfate, filtered and concentrated under reduced pressure. The residue was chromatographed on a silica gel column with EA:PE (8:92) to afford ethyl (2S)-2-((t-butoxycarbonyl)amino)-3,4,4-trimethylpentanoate (85 mg, crude) as a brown oil. LC-MS (ESI, m/z): 188 [M-100+H]⁺.

To a mixture of ethyl (2S)-2-((tert-butoxycarbonyl)amino)-3,4,4-trimethylpentanoate (85.0 mg, 0.296 mmol) in MeOH (1 mL)/THF (1 mL) was added a solution of lithium hydroxide (50.0 mg, 2.07 mmol) in water (1 mL). The mixture was stirred overnight at rt. The mixture was concentrated under reduced pressure to remove the THF:MeOH and adjusted to pH=6 with HCl (1 M). The mixture was extracted with EA (3×5 mL). The organic layers were combined, washed with brine (2×3 mL), dried over anhydrous sodium sulfate, filtered and concentrated under reduced pressure to afford (2S)-2-((tert-butoxycarbonyl)amino)-3,4,4-trimethylpentanoic acid (77 mg, crude) as a yellow oil. LC-MS (ESI, m/z): 204 [M-56+H]⁺.

### Example 80

Additional Compounds

Additional compounds of Formula (I) can be prepared using similar materials and methods described herein, such as those described herein.

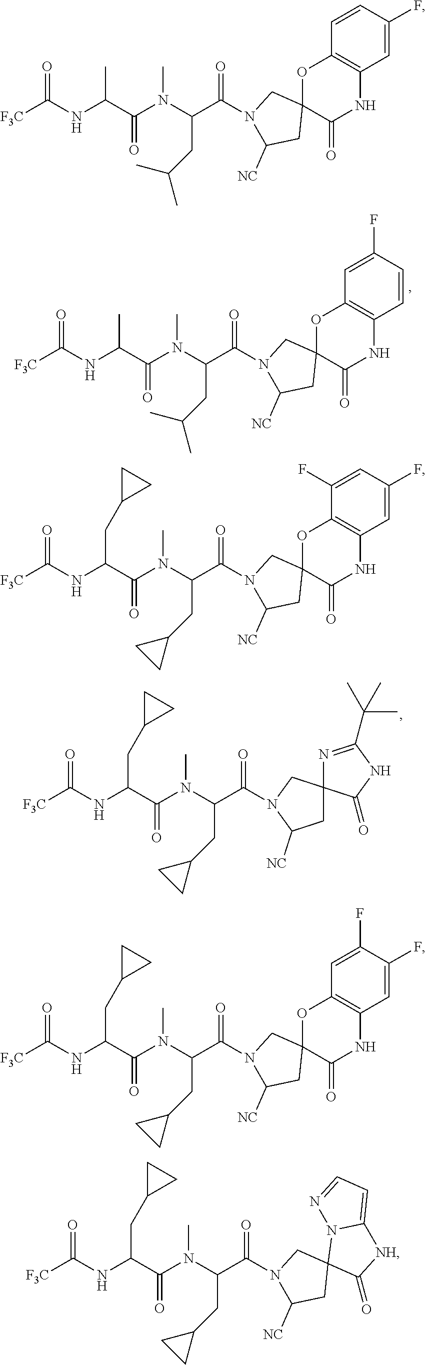

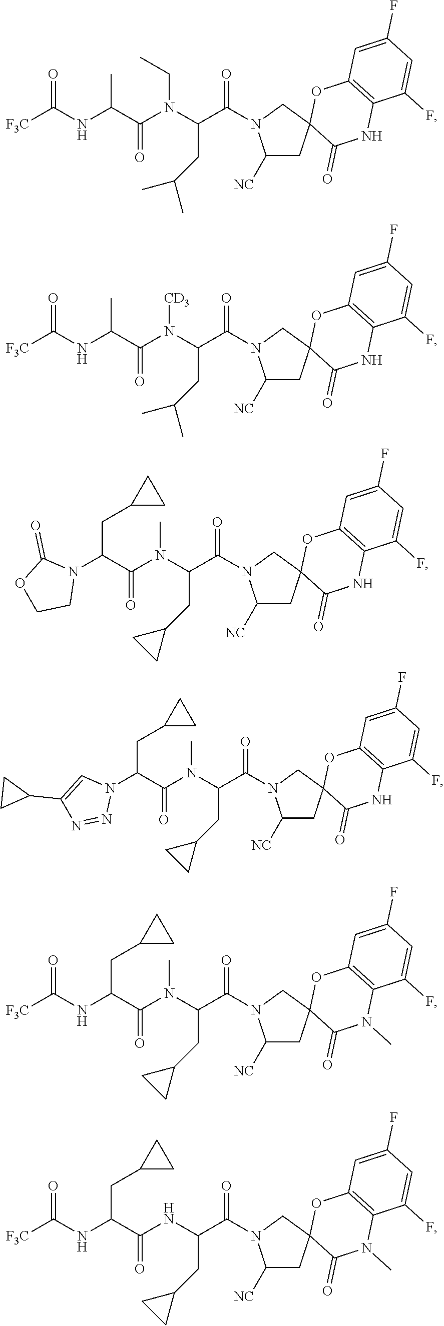

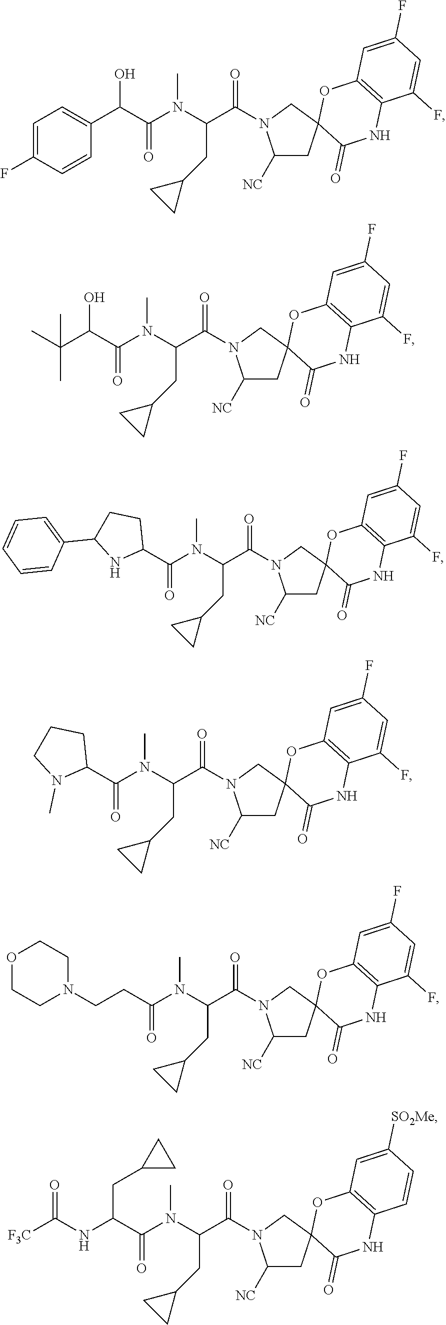 [SMILES: CN(C(=O)C(O)c1ccc(F)cc1)C(CC1CC1)C(=O)N1CC2(CC1C#N)Oc1cc(F)cc(F)c1NC2=O ; CN(C(=O)C(O)C(C)(C)C)C(CC1CC1)C(=O)N1CC2(CC1C#N)Oc1cc(F)cc(F)c1NC2=O ; CN(C(=O)C1CCC(c2ccccc2)N1)C(CC1CC1)C(=O)N1CC2(CC1C#N)Oc1cc(F)cc(F)c1NC2=O ; CN1CCCC1C(=O)N(C)C(CC1CC1)C(=O)N1CC2(CC1C#N)Oc1cc(F)cc(F)c1NC2=O ; CN(C(=O)CCN1CCOCC1)C(CC1CC1)C(=O)N1CC2(CC1C#N)Oc1cc(F)cc(F)c1NC2=O ; CN(C(=O)C(CC1CC1)NC(=O)C(F)(F)F)C(CC1CC1)C(=O)N1CC2(CC1C#N)Oc1cc(S(C)(=O)=O)ccc1NC2=O]

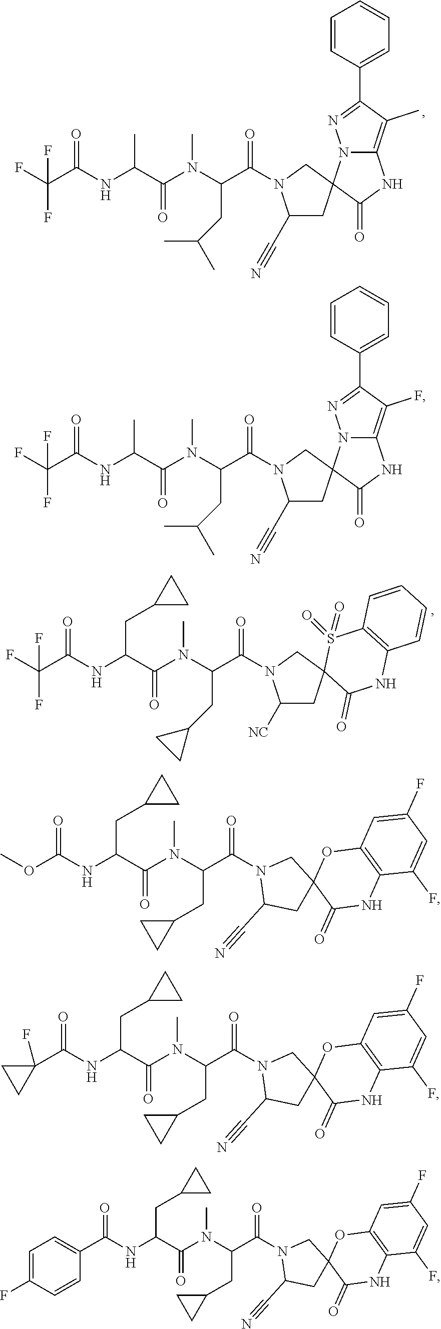

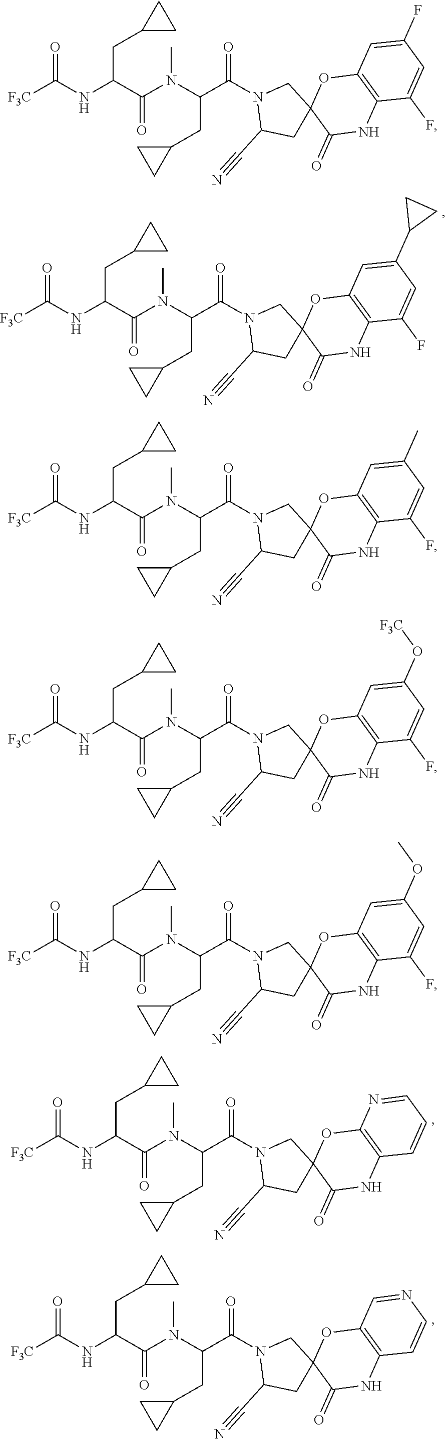

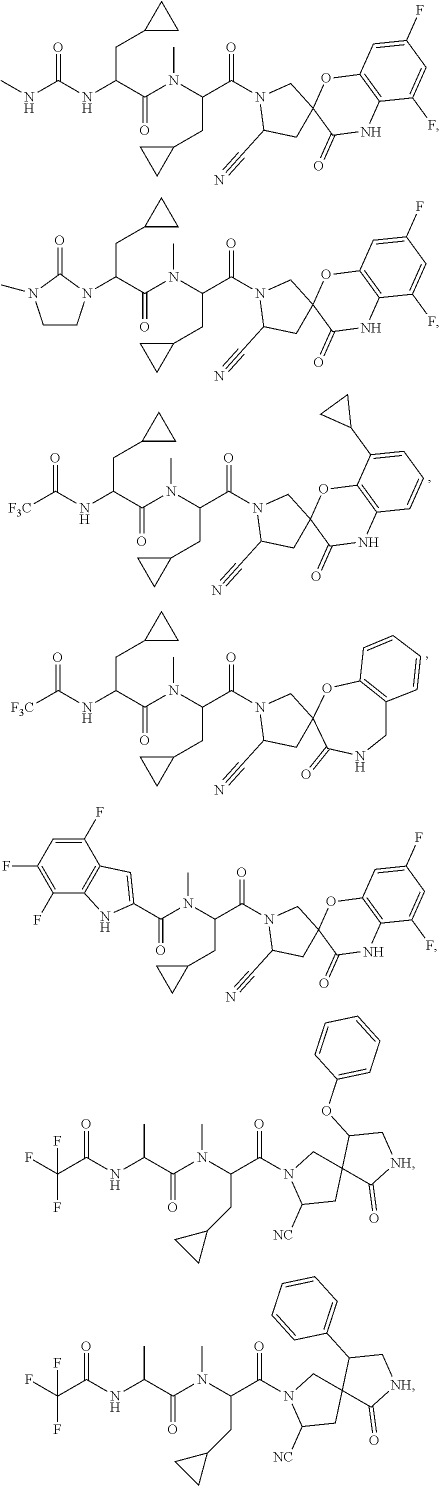

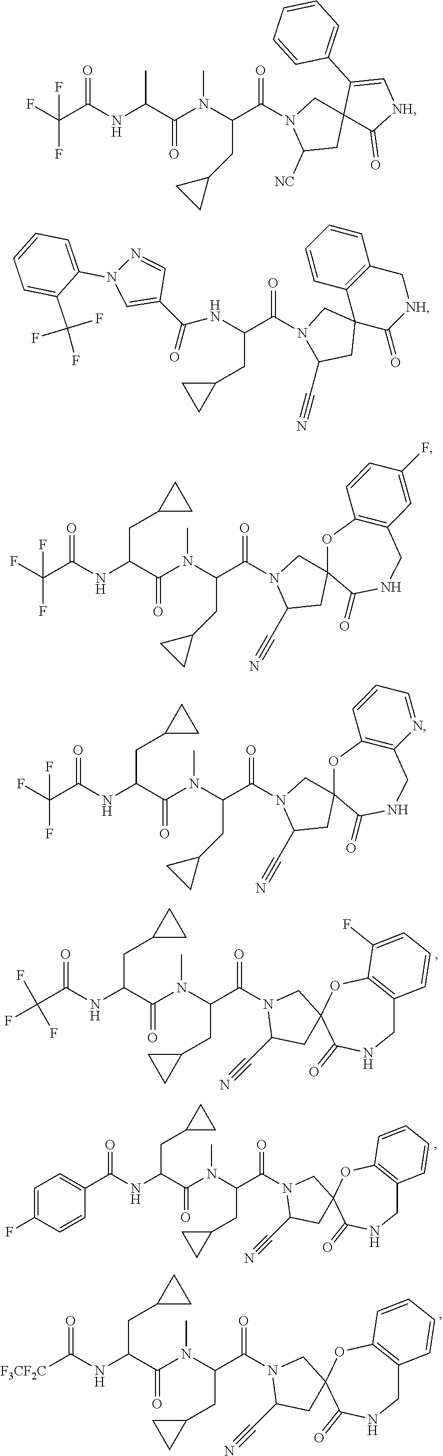

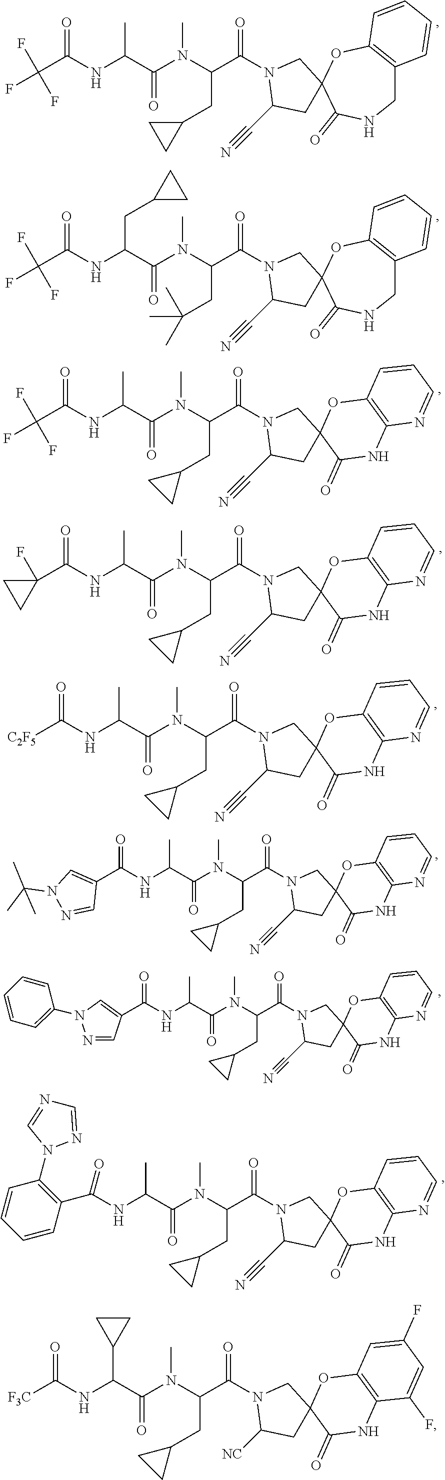

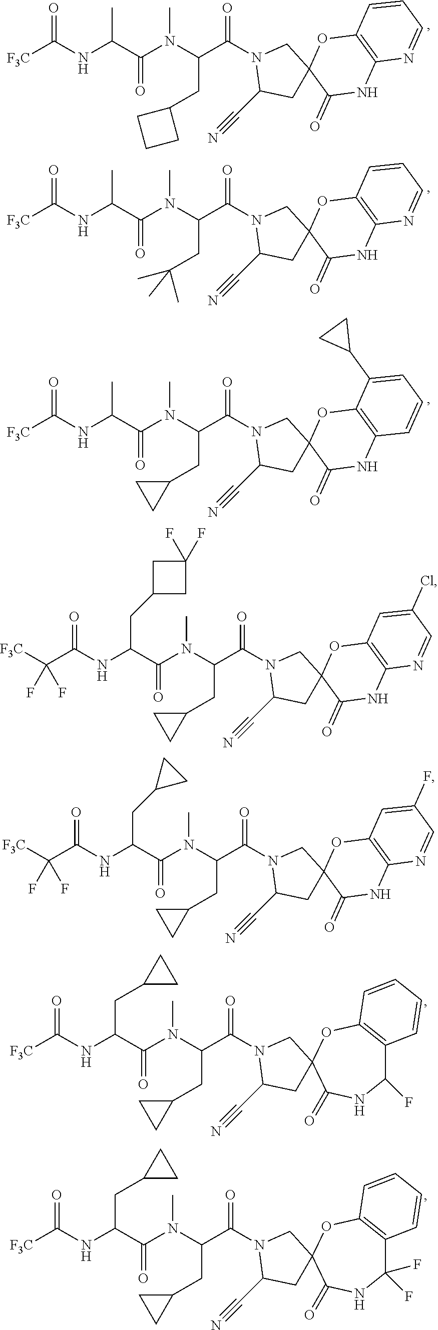

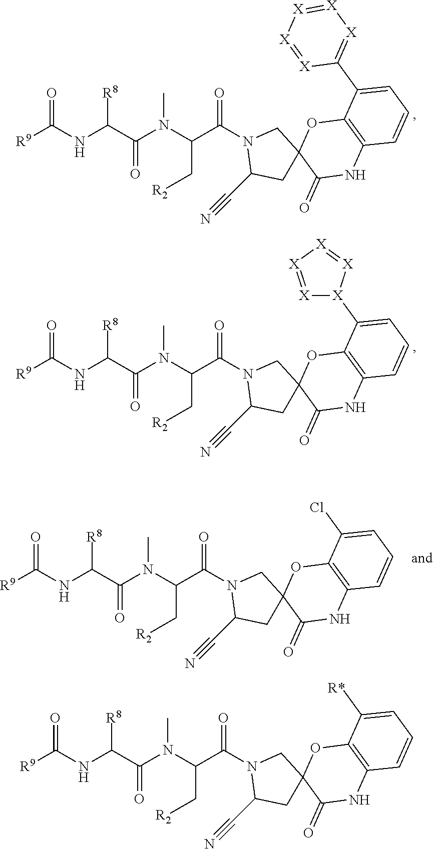

(X═CH or N, and R*=an unsubstituted C₁₋₄ alkyl or an unsubstituted —O(an unsubstituted C₁₋₄ alkyl)) (including pharmaceutically acceptable salts thereof).

LC-MS Methods

| Compound | Rt | | LC-MS |
|---|---|---|---|
| No. | (min) | MS | Method |
| 1 | 1.494 | [M + H]⁺ = 295 (fragment peak) | F |
| 2 | 1.210 | [M + H]⁺ = 307 (fragment peak) | E |
| 3 | 0.850 | [M + H]⁺ = 537 | B |
| 4 | 1.525 | [M + H]⁺ = 550 | D |
| 5 | 1.149 | [M + NH₃]⁺ = 553 | C |
| 6 | 0.962 | [M + H]⁺ = 547 | B |
| 7 | 1.294 | [M + H]⁺ = 498 | G |
| 8 | 1.960 | [M + H]⁺ = 460 | A |
| 9 | 2.048 | [M + H]⁺ = 474 | A |
| 10 | 1.999 | [M + H]⁺ = 489 | A |
| 11 | 1.992 | [M + H]⁺ = 474 | A |
| 12 | 0.785 | [M + H]+ = 535 | H |
| 13 | 2.062 | [M + H]⁺ = 486 | A |
| 14a | 0.858 | [M + Na]+ = 564 | H |
| 14b | 0.885 | [M + Na]+ = 564 | H |
| 15 | 0.916 | [M + H]+ = 333(fragment peak) | H |
| 16 | 1.999 | [M + H]+ = 499 | A |
| 17 | 2.042 | [M + H]+ = 549 | A |
| 18 | 1.976 | [M + H]+ = 473 | A |
| 19 | 2.076 | [M + H]+ = 514 | A |
| 20 | 2.069 | [M + H]+ = 500 | A |
| 21 | 1.993 | [M + H]+ = 474 | A |
| 22 | 2.033 | [M + H]+ = 488 | A |
| 23 | 2.051 | [M + H]+ = 488 | A |
| 24 | 2.13 | [M − H]− = 534 | A |
| 25 | 0.91 | [M + Na]+ = 596 | H |
| 26 | 0.841 | [M + Na]+ = 598 | H |
| 27 | 4.95 | [M + H]+ = 548 | J |
| 28 | 1.572 | [M + H]+ = 552 | G |
| 29 | 1.54 | [[M + H]+ = 295(fragment peak) | G |
| 30 | 0.996 | [M + H]+ = 325(fragment peak) | H |
| 31 | 1.483 | [M + H]+ = 536 | K |
| 32 | 0.825 | [M + Na]+ = 544 | H |
| 33 | 0.976 | [[M + H]+ = 333(fragment peak) | L |
| 34 | 1.455 | [M + H]+ = 333(fragment peak) | M |
| 35 | 0.952 | [M + H]+ = 333(fragment peak) | L |
| 36 | 0.983 | [[M + H]+ = 335(fragment peak) | L |
| 37 | 0.875 | [M + H]+ = 293(fragment peak) | L |
| 38 | 2.105 | [M − H]− = 500 | A |
| 39 | 2.243 | [M − H]− = 576 | A |
| 40 | 0.785, 0.800 | [M + H]+ = 590 | H |
| 41 | 0.697, 0.711 | [M + H]+ = 512 | H |
| 42 | 0.868 | [M − H]− = 535 | L |
| 43 | 0.896 | [M + Na]+ = 580 | H |
| 44 | 0.789 | [M + H]+ = 512 | H |
| 45 | 1.278 | [M + Na]+ = 520 | N |
| 46 | 0.626 | [M + H]+ = 512 | L |
| 47 | 1.132, 1.157 | [M + H]+ = 515 | P |
| 48 | 0.883, 0.903 | [M + Na]+ = 582 | H |
| 49 | 0.88 | [M + Na]+ = 582 | H |
| 50 | 1.104 | [M + H]+ = 333(fragment peak) | Q |
| 51 | 1.359 | [M + Na]+ = 582 | R |
| 52 | 1.492 | [M + Na]+ = 620 | S |
| 53 | 0.998 | [M − H]− = 598 | L |
| 54 | 0.949 | [[M + H]+ = 353(fragment peak) | L |
| 55 | 0.956 | [M + H]+ = 333(fragment peak) | L |
| 56 | 0.938 | [M + H]+ = 333(fragment peak) | L |
| 57 | 0.906 | [M + Na]+ = 620 | H |
| 58 | 0.956 | [M + Na]+ = 598 | L |
| 59 | 0.992 | [M + H]+ = 333(fragment peak) | L |
| 60 | 2.12 | [M + H]+ = 524 | A |
| 61 | 0.847 | [M + Na]+ = 600 | H |
| 62 | 0.915 | [M + Na]+ = 596 | H |
| 63 | 1.023 | [M + Na]+ = 624 | L |
| 64 | 1.208 | [M + H]+ = 355(fragment peak) | T |
| 65 | 0.912 | [M + H]+ = 295(fragment peak) | U |
| 66 | 0.876 | [M + H]+ = 333(fragment peak) | L |
| 67 | 0.905 | [M + H]+ = 333(fragment peak) | L |
| 68 | 0.821 | [M + Na]+ = 568 | H |
| 69 | 1.005 | [M + Na]+ = 676 | L |
| 70 | 1.37 | [M + H]+ = 257(fragment peak) | G |
| 71 | 1.251 | [M + H]+ = 285(fragment peak) | V |
| 72 | 0.905 | [M + Na]+ = 612 | L |
| 73 | 0.734 | [M + H]+ = 563 | W |
| 74 | 0.977 | [M + Na + CH3CN]+ = 651 | H |
| 75 | 1.556 | [M + H]+ = 295(fragment peak) | G |
| 76 | 1.062 | [M + H]+ = 323(fragment peak) | T |
| 77 | 0.931 | [M + Na]+ = 646 | H |
| 78 | 0.905 | [M + Na]+ = 606 | H |
| 79 | 1.665 | [M + H]+ = 629 | X |
| 80 | 1.355 | [M + Na]+ = 574 | N |
| 81 | 0.883 | [M + Na]+ = 598 | H |
| 82 | 2.243 | [M + H]+ = 610 | A |
| 83 | 0.872 | [M + Na]+ = 580 | H |
| 84 | 0.767 | [M + Na]+ = 545 | H |
| 85 | 0.947 | [M + Na]+ = 648 | H |
| 86 | 0.919 | [M + Na]+ = 630 | H |
| 87 | 1.011 | [M + Na]+ = 634 | H |
| 88 | 0.923 | [M + Na]+ = 594 | H |
| 89 | 1.427 | [M + Na]+ = 636 | W |
| 90 | 0.997 | [M + Na]+ = 674 | H |
| 91 | 0.813 | [M + Na]⁺ = 585 | H |
| 92 | 1.529 | [M + Na]⁺ = 594 | X |
| 93 | 2.374 | [M − H]⁻ = 610 | A |
| 94 | 0.789 | [M + Na]⁺ = 572 | H |
| 95 | 0.950 | [M + Na]⁺ = 630 | H |
| 96 | 0.894 | [M + H]⁺ = 323(fragment peak) | Y |
| 97 | 0.977 | [M + Na]⁺ = 633 | H |
| 98 | 0.812 | [M + Na]⁺ = 562 | H |
| 99 | 1.411 | [M + Na]⁺ = 584 | R |
| 100 | 1.011 | [M + Na]⁺ = 636 | H |
| 101 | 2.124 | [M − H]⁻ = = 570 | A |
| 102 | 2.200 | [M − H]⁻ = = 604 | A |
| 103 | 0.855 | [M + Na]⁺ = 624 | H |
| 104 | 0.789 | [M + Na]⁺ = 572 | H |
| 105 | 1.205 | [M + NH3 + H]⁺ = 580 | Z |
| 106 | 2.225 | [M − H]⁻ = 572 | A |
| 107 | 2.197 | [M + H]⁺ = 593 | A |
| 108 | 2.186 | [M + H]⁺ = 593 | A |
| 109 | 2.252 | [M + H]⁺ = 563 | A |
| 110 | 2.106 | [M + H]⁺ = 640 | A |
| 111 | 2.011 | [M + H]⁺ = 564 | A |
| 112 | 2.279 | [M + H]⁺ = 633 | A |
| 113 | 2.390 | [M − H]⁻ = 600 | A |
| 114 | 2.430 | [M + H]⁺ = 630 | A |
| 115 | 2.279 | [M − H]⁻ = 588 | A |
| 116 | 2.029 | [M + H]⁺ = 556 | A |
| 117 | 2.261 | [M + H]⁺ = 582 | A |
| 118 | 2.270 | [M + H]⁺ = 582 | A |
| 119 | 1.186 | [M + Na]⁺ = 622 | W |
| 120 | 0.893 | [M + Na]⁺ = 570 | H |
| 121 | 0.783 | [M + Na]⁺ = 599 | H |
| 122 | 0.864 | [M + Na]⁺ = 582 | H |
| 123 | 0.878 | [M + Na]⁺ = 582 | H |
| 124 | 0.984 | [M + Na]⁺ = 609 | H |
| 125 | 1.010 | [M + Na]⁺ = 660 | H |
| 126 | 0.829 | [M + Na]⁺ = 599 | H |
| 127 | 1.644 | [M + Na]⁺ = 606 | R |
| 128 | 2.135 | [M − H]⁻ = 582 | A |
| 129 | 2.155 | [M − H]⁻ = 582 | A |
| 130 | 2.256 | [M + H]⁺ = 588 | A |
| 131 | 1.878 | [M + H]⁺ = 502 | A |
| 132 | 2.668 | [M + H]⁺ = 532 | A |
| 133 | 2.453 | [M + H]⁺ = 568 | A |
| 134 | 2.360 | [M + H]⁺ = 564 | A |
| 135 | 2.433 | [M + H]⁺ = 598 | A |
| 136 | 2.252; 2.316 | [M + H]⁺ = 543 | A |
| 137 | 2.218 | [M + H]⁺ = 535 | A |
| 138 | 2.308; 2.365 | [M + H]⁺ = 505 | A |
| 139 | 2.201 | [M − H]⁻ = 536 | A |
| 140 | 2.371 | [M − H]⁻ = 564 | A |
| 141 | 2.336 | [M − H]⁻ = 592 | A |
| 142 | 2.367 | [M − H]⁻ = 592 | A |
| 143 | 2.246 | [M + H]⁺ = 675 | A |
| 144 | 2.215 | [M − H]⁻ = 539 | A |
| 145 | 2.433 | [M + H]⁺ = 605 | A |
| 146 | 2.227 | [M + H]⁺ = 568 | A |
| 147 | 2.211 | [M − H]⁻ = = 555 | A |
| 148 | 1.924 | [M + H]⁺ = 600 | A |
| 149 | 2.266 | [M + H]⁺ = 629 | A |
| 150 | 2.433 | [M + H]⁺ = 610 | A |
| 151 | 1.968 | [M + H]⁺ = 600 | A |
| 152 | 2.305 | [M + H]⁺ = 629 | A |
| 153 | 2.121 | [M + H]⁺ = 578 | A |
| 154 | 2.340 | [M − H]⁻ = 602 | A |
| 155 | 2.093 | [M + H]⁺ = 519 | A |
| 156 | 2.351 | [M + H]⁺ = 595 | A |
| 157 | 2.426 | [M − H]⁻ = = 620 | A |
| 158 | 1.234 | [M + Na]⁺ = 652 | W |
| 159 | 1.523 | [M + Na]⁺ = 666 | AA |
| 160 | 0.801 | [M + Na]⁺ = 544 | H |
| 161 | 0.946 | [M + Na]⁺ = 648 | H |
| 162 | 1.227 | [M + Na]⁺ = 558 | R |
| 163 | 1.227 | [M + Na]⁺ = 558 | R |
| 164 | 0.934 | [M + Na]⁺ = 614 | H |
| 165 | 0.782 | [M + Na]⁺ = 559 | H |
| 166 | 0.798 | [M + Na]⁺ = 561 | H |
| 167 | 0.877 | [M + Na]⁺ = 616 | H |
| 168 | 0.862 | [M + Na]⁺ = 616 | H |
| 169 | 0.674 | [M + Na]⁺ = 535 | H |
| 170 | 0.798 | [M + Na]⁺ = 595 | H |
| 171 | 0.874 | [M + Na]⁺ = 575 | H |
| 172 | 0.895 | [M + Na]⁺ = 585 | H |
| 173 | 0.994 | [M + Na]⁺ = 599 | H |
| 174 | 0.808 | [M + Na]⁺ = 561 | H |
| 175 | 1.493 | [M + H]⁺ = 341(fragment peak) | X |
| 176 | 0.893 | [M + Na]⁺ = 588 | L |
| 177 | 0.861 | [M + Na]⁺ = 573 | H |
| 178 | 0.823 | [M + Na]⁺ = 595 | L |
| 179 | 1.443 | [M + Na]⁺ = 571 | X |
| 180 | 1.070 | [M + Na]⁺ = 531 | X |
| 181 | 1.410 | [M + Na]⁺ = 599 | X |
| 182 | 1.068 | [M + H]⁺ = 333(fragment peak) | AB |
| 183 | 1.451 | [M + Na]⁺ = 559 | G |
| 184 | 0.871 | [M + H]⁺ = 613 | H |
| 185 | 0.892 | [M + H]⁺ = 553 | AB |
| 186 | 1.494 | [M + H]⁺ = 359(fragment peak) | G |
| 187 | 1.464 | [M + Na]⁺ = 635 | AG |
| 188 | 1.139 | [M + Na]⁺ = 601 | W |
| 189 | 1.372 | [M + Na]⁺ = 613 | R |
| 190 | 0.927 | [M + H]⁺ = 365(fragment peak) | L |
| 191 | 1.119 | [M + Na]⁺ = 651 | W |
| 192 | 1.219 | [M + Na]⁺ = 665 | W |
| 193 | 1.431 | [M + Na]⁺ = 635 | R |
| 194 | 0.885 | [M + Na]⁺ = 606 | H |
| 195 | 0.737 | [M + Na]⁺ = 571 | H |
| 196 | 1.087 | [M + H]⁺ = 323(fragment peak) | AB |
| 197 | 0.964 | [M + Na]⁺ = 647 | W |
| 198 | 0.815 | [M + Na]⁺ = 607 | H |
| 199 | 0.953 | [M + Na]⁺ = 634 | L |
| 200 | 1.533 | [M + Na]⁺ = 639 | R |
| 201 | 0.832 | [M + Na]⁺ = 621 | H |
| 202 | 0.860 | [M + Na]⁺ = 637 | H |
| 203 | 0.843 | [M + H]⁺ = 577 | H |
| 204 | 0.918 | [M + H]⁺ = 579 | L |
| 205 | 0.858 | [M + H]⁺ = 617 | H |
| 206 | 0.881 | [M + Na]⁺ = 613 | H |
| 207 | 1.203 | [M + Na]⁺ = 634 | W |
| 208 | 0.849 | [M + H]⁺ = 526 | W |
| 209 | 0.689 | [M + Na]⁺ = 545 | H |
| 210 | 0.774 | [M + H]⁺ = 527 | H |
| 211 | 1.224 | [M + H]⁺ = 526 | G |
| 212 | 0.784 | [M + Na]⁺ = 545 | L |
| 213 | 1.321 | [M + Na]⁺ = 507 | X |
| 214 | 0.679 | [M + H]⁺ = 525 | H |
| 215 | 1.093 | [M + H]⁺ = 361 (fragment peak) | AB |
| 216 | 0.747 | [M + H]⁺ = 291 (fragment peak) | L |
| 217 | 0.929 | [M + H]⁺ = 345(fragment peak) | L |
| 218 | 0.860 | [M + Na]⁺ = 599 | H |
| 219 | 0.684 | [M + Na]⁺ = 533 | H |
| 220 | 0.837 | [M + H]⁺ = 337(fragment peak) | L |
| 221 | 0.602 | [M + H]⁺ = 580 | L |
| 222 | 1.136 | [M + Na]⁺ = 612 | W |
| 223 | 0.960 | [M + Na]⁺ = 612 | L |
| 224 | 0.960 | [M + Na]⁺ = 612 | L |
| 225 | 1.431 | [M + Na]⁺ = 550 | X |
| 226 | 0.860 | [M + Na]⁺ = 543 | L |
| 227 | 0.817 | [M + H]⁺ = 577 | H |
| 228 | 1.003 | [M + Na]⁺ = 626 | L |
| 229 | 4.51 | [M + Na]⁺ = 559 | AI |
| 230 | 0.572 | [M + Na]⁺ = 555 | L |
| 231 | 0.626 | [M + Na]⁺ = 500 | L |
| 232 | 0.684 | [M + H]⁺ = 491 | L |
| 233 | 2.381 | [M + H]⁺ = 579 | A |
| 234 | 1.122 | [M + H]⁺ = 363(fragment peak) | AB |
| 235 | 1.152 | [M + H]⁺ = 628 | Q |
| 236 | 1.051 | [M + H]⁺ = 566 | AH |
| 237 | 1.371 | [M + H]⁺ = 527 | AH |
| 238 | 0.823 | [M + H]⁺ = 579 | H |
| 239 | 1.349 | [M + Na]⁺ = 589 | X |
| 240 | 1.246 | [M + H]⁺ = 567 | X |
| 241 | 2.077 | [M + H]⁺ = 570 | A |
| 242 | 0.769 | [M + H]⁺ = 613 | AC |
| 243 | 0.750 | [M + H]⁺ = 559 | H |
| 244 | 1.132 | [M + H]⁺ = 585 | R |
| 245 | 1.246 | [M + H]⁺ = 573 | AD |
| 246 | 0.698 | [M + H]⁺ = 539 | AC |
| 247 | 0.960 | [M + H]⁺ = 589 | AB |
| 248 | 1.344 | [M + H]⁺ = 433(fragment peak) | AE |
| 249 | 1.588 | [M + H]⁺ = 393(fragment peak) | AF |

Final compounds can be obtained in some cases as a mixture with a corresponding stereoisomer. Retention times of the main isomers are depicted in the table above.

Description of LC-MS methods

| | | | | | | | Run |
|---|---|---|---|---|---|---|---|
| LC | | | | | Flow | Col T | Time |
| Method | Instrument | Column | Mobile Phase | Gradient | mL/min | (° C.) | (min) |
| A | Agilent | Acquity UPLC BEH | A: 0.1% FA in Water, | 98% A held for 0.2 min, | 0.6 | 70 | 3.8 |
| | 6150 SQ- | C18 (1.7 μm, 2.1 × 50 | B: 0.1% FA in | to 2% A in 1.3 min, held | | | |
| | Agilent | mm) | Acetonitrile | for 1.8 min, to 98% A in | | | |
| | 1290 | | | 0.1 min, held for 0.4 | | | |
| | Infinity | | | min | | | |
| B | Shimadzu | HALO 2.0 μm C18 | A: Water/0.1% FA | From 95% A to 0% A in | 1.5 | 40 | 1.85 |
| | LCMS-2020 | 90A | B: Acetonitrile/0.1% | 1.09 min, held for 0.6 | | | |
| | | (2.0 μm, 3.0 × 30 mm) | FA | min, to 95% A in 0.05 | | | |
| | | | | min, held for 0.10 min | | | |
| C | Shimadzu | Shim-Pack C18 (3 μm, | A: Water/5 mM | From 90% A to 5% A in | 1.5 | 40 | 2 |
| | LCMS-2020 | 3.0 × 33 mm) | NH₄HCO₃ | 1.19 min, held for 0.6 | | | |
| | | | B: Acetonitrile | min, to 90% A in 0.02 | | | |
| | | | | min, held for 0.18 min | | | |
| D | Shimadzu | HALO 2.0 μm C18 | A: Water/0.1% FA | From 80% A to 30% A | 1.5 | 40 | 3 |
| | LCMS-2020 | 90A | B: ACN/0.1% FA | in 1.69 min, to 100% A | | | |
| | | (2.0 μm, 3.0 × 30 mm) | | in 0.6 min, held for 0.5 | | | |
| | | | | min, to 95% A in 0.03 | | | |
| | | | | min, held for 0.17 min | | | |
| E | Shimadzu | HALO 2.0 μm C18 | A: Water/0.1% FA | From 70% A to 5% A in | 1.5 | 40 | 3 |
| | LCMS-2020 | 90A | B: ACN/0.1% FA | 2.09 min, held for 0.6 | | | |
| | | (2.0 μm, 3.0 × 30 mm) | | min, to 95% A in 0.05 | | | |
| | | | | min, held for 0.25 min | | | |
| F | Shimadzu | HALO C18 | A: Water + 0.05% FA | From 95% A to 35% A | 1.5 | 40 | 3 |
| | LCMS-2020 | (2.0 μm, 3.0 × 30 mm) | B: ACN + 0.05% FA | in 1.69 min, to 5% A in | | | |
| | | | | 0.6 min, held for | | | |
| | | | | 0.5 min, to 95% A in | | | |
| | | | | 0.03 min, held for 0.17 | | | |
| | | | | min | | | |
| G | Shimadzu | HALO 2.0 μm C18 | A: Water/0.1% FA | From 95% A to 40% A | 1.2 | 40 | 3 |
| | LCMS-2020 | 90A | B: ACN/0.1% FA | in 1.69 min, to 0% A in | | | |
| | | (2.0 μm, 3.0 × 30 mm) | | 0.6 min, held for | | | |
| | | | | 0.5 min, to 95% A in | | | |
| | | | | 0.03 min, held for 0.17 | | | |
| | | | | min | | | |
| H | Shimadzu | HALO C18 | A: Water + 0.05% TFA | From 95% A to 0% A in | 1.5 | 40 | 2 |
| | LCMS-2020 | (2.0 μm, 3.0 × 30 mm) | B: ACN + 0.05% TFA | 1.19 min, held for 0.6 | | | |
| | | | | min, to 95% A in 0.02 | | | |
| | | | | min, held for 0.18 min | | | |
| J | Shimadzu | HALO C18 | A: Water/0.05% TFA | From 90% A to 5% A in | 1.5 | 40 | 10 |
| | LCMS-2020 | (2.7 μm, 100 × 4.6 | B: CH₃CN/0.05% TFA | 8 min, held for 2 min. | | | |
| | | mm) | | | | | |
| K | Shimadzu | HALO C18 | A: Water + 0.05% FA | From 95% A to 50% A | 1.2 | 40 | 3 |
| | LCMS-2020 | (2.0 μm, 3.0 × 30 mm) | B: CH₃CN + 0.05% FA | in 1.69 min, to 0% A in | | | |
| | | | | 0.6 min, held for 0.5 | | | |
| | | | | min, to 95% A in 0.03 | | | |
| | | | | min, held for 0.17 min | | | |
| L | Shimadzu | HALO | A: Water/0.1% FA | From 95% A to 0% A in | 1.2 | 40 | 2 |
| | LCMS-2020 | (2.0 μm, 3.0 × 30 mm) | B: CH₃CN/0.1% FA | 1.19 min, held for 0.6 | | | |
| | | | | min, to 95% A in 0.02 | | | |
| | | | | min, held for 0.18 min. | | | |
| M | Shimadzu | HALO | A: Water/0.1% FA | From 80% A to 40% A | 1.2 | 40 | 3 |
| | LCMS-2020 | (2.0 μm, 3.0 × 30 mm) | B: CH₃CN/0.1% FA | in 1.69 min, to 0% A in | | | |
| | | | | 0.6 min, held for 0.5 | | | |
| | | | | min, to 95% A in 0.03 | | | |
| | | | | min, held for 0.17 min | | | |
| N | Shimadzu | HALO 90 A C18 | A: Water/0.05% TFA | From 95% A to 40% A | 1.5 | 40 | 3 |
| | LCMS-2020 | (2.0 μm, 3.0 × 30 mm) | B: CH₃CN/0.05% TFA | in 1.69 min, to 5% A in | | | |
| | | | | 0.6 min, held for 0.5 | | | |
| | | | | min, to 95% A in 0.03 | | | |
| | | | | min, held for 0.17 min | | | |
| P | Shimadzu | HALO C18 | A: Water + 0.05% TFA | From 95% A to 30% A | 1.5 | 40 | 3 |
| | LCMS-2020 | (2.0 μm, 3.0 × 30 mm) | B: CH₃CN + 0.05% | in 1.69 min, to 5% A in | | | |
| | | | TFA | 0.6 min, held for 0.5 | | | |
| | | | | min, to 95% A in 0.03 | | | |
| | | | | min, held for 0.17 min | | | |
| Q | Shimadzu | HALO 90A C18 | A: Water/0.1% FA | From 70% A to 30% A | 1.2 | 40 | 3 |
| | LCMS-2020 | (2.0 μm, 3.0 × 30 mm) | B: CH₃CN/0.1% FA | in 1.69 min, to 0% A in | | | |
| | | | | 0.6 min, held for 0.5 | | | |
| | | | | min, to 95% A in 0.03 | | | |
| | | | | min, held for 0.17 min | | | |
| R | Shimadzu | HALO (2.0 μm, 3.0 × | A: Water/0.1% TFA | From 80% A to 40% A | 1.5 | 40 | 3 |
| | LCMS-2020 | 30 mm) | B: CH₃CN/0.1% TFA | in 1.69 min, to 5% A in | | | |
| | | | | 0.6 min, held for 0.5 | | | |
| | | | | min, to 95% A in 0.03 | | | |
| | | | | min, held for 0.17 min | | | |
| S | Shimadzu | HALO 2.0 μm C18 | A: Water/0.1% FA | From 95% A to 30% A | 1.5 | 40 | 3 |
| | LCMS-2020 | 90A | B: CH₃CN/0.1% FA | in 1.69 min, to 0% A in | | | |
| | | (2.0 μm, 3.0 × 30 mm) | | 0.6 min, held for | | | |
| | | | | 0.5 min, to 95% A in | | | |
| | | | | 0.03 min, held for | | | |
| | | | | 0.17 min | | | |
| T | Shimadzu | HALO | A: Water/0.1% FA | From 70% A to 30% A | 1.2 | 40 | 3 |
| | LCMS-2020 | (2.0 μm, 3.0 × 30 mm) | B: CH₃CN/0.1% FA | in 1.69 min, held for | | | |
| | | | | 0.6 min, to 0% A in | | | |
| | | | | 0.5 min, to 95% A in | | | |
| | | | | 0.03 min, held for | | | |
| | | | | 0.17 min. | | | |
| U | Shimadzu | Shim - pack Scepter | A: Water - 5 mM | From 90% A to 5% A in | 1.5 | 40 | 2 |
| | LCMS-2020 | C18 - 120 | NH₄HCO₃ | 1.19 min, held for 0.6 | | | |
| | | (3.0 μm, 3.0 × 33 mm) | B: CH₃CN | min, to 90% A in 0.02 | | | |
| | | | | min, held for 0.18 min. | | | |
| V | Shimadzu | HALO 2.0 μm C18 | A: Water/0.1% FA | From 80% A to 40% A | 1.5 | 40 | 3 |
| | LCMS-2020 | 90A | B: ACN/0.1% FA | in 1.69 min, to 0% A in | | | |
| | | (2.0 μm, 3.0 × 30 mm) | | 0.6 min, held for 0.5 | | | |
| | | | | min, to 95% A in 0.03 | | | |
| | | | | min, held for 0.17 min | | | |
| W | Shimadzu | HALO 2.0 μm C18 | A: Water/0.1% TFA | From 70% A to 30% A | 1.5 | 40 | 3 |
| | LCMS-2020 | 90A | B: ACN/0.1% TFA | in 1.69 min, to 5% A in | | | |
| | | (2.0 μm, 3.0 × 30 mm) | | 0.6 min, held for | | | |
| | | | | 0.5 min, to 95% A in | | | |
| | | | | 0.03 min, held for 0.17 | | | |
| | | | | min | | | |
| X | Shimadzu | HALO C18 | A: Water + 0.05% TFA | From 95% A to 40% A | 1.5 | 40 | 3 |
| | LCMS-2020 | (2.0 μm, 3.0 × 30 mm) | B: ACN + 0.05% TFA | in 1.69 min, to 5% A in | | | |
| | | | | 0.6 min, held for 0.5 | | | |
| | | | | min, to 95% A in 0.03 | | | |
| | | | | min, held for 0.17 min | | | |
| Y | Shimadzu | kinetex EVO-C18 | A: Water/6.5 mM | From 90% A to 5% A in | 1.5 | 40 | 2 |
| | LCMS-2020 | (2.6 μm, 3.0 × 30 mm) | NH₄HCO₃ + NH₄OH | 1.19 min, held for | | | |
| | | | (pH = 10), B: CH₃CN | 0.6 min, to 90% A in | | | |
| | | | | 0.02 min, held for 0.18 | | | |
| | | | | min | | | |
| Z | Shimadzu | Shim - pack Scepter | A: 10% CH₃CN | From 100% A to 10% A | 1.5 | 40 | 2 |
| | LCMS-2020 | C18 - 120 | 90% Water/5 mM | in 1.69 min, held for 0.6 | | | |
| | | (3.0 μm, 3.0*33 mm) | NH₄HCO₃ | min, to 100% A in 0.00 | | | |
| | | | B: CH₃CN | min, held for 0.18 min | | | |
| AA | Shimadzu | HALO 2.0 μm C18 | A: Water/0.1% TFA | From 80% A to 30% A | 1.5 | 40 | 3 |
| | LCMS-2020 | 90A | B: CH₃CN/0.1% TFA | in 1.69 min, to 0% A in | | | |
| | | (2.0 μm, 3.0*30 mm) | | 0.6 min, held for 0.5 | | | |
| | | | | min, to 95% A in 0.03 | | | |
| | | | | min, held for 0.17 min | | | |
| AB | Shimadzu | Shim - pack Scepter | A: 5% CH₃CN | From 95% A to 10% A | 1.5 | 40 | 2 |
| | LCMS-2020 | C18 - 120 | 95% Water/5 mM | in 1.19 min, held for 0.6 | | | |
| | | (3.0 μm, 3.0*33 mm) | NH₄HCO₃ | min, to 95% A in 0.02 | | | |
| | | | B: CH₃CN | min, held for 0.18 min | | | |
| AC | Shimadzu | kinetex EVO-C18 | A: Water/6.5 mM | From 90% A to 10% A | 1.5 | 40 | 2 |
| | LCMS-2020 | (2.6 μm, 3.0*30 mm) | NH₄HCO₃ | in 1.19 min, held for | | | |
| | | | B: CH₃CN | 0.6 min, to 90% A in | | | |
| | | | | 0.02 min, held for | | | |
| | | | | 0.18 min | | | |
| AD | Shimadzu | kinetex EVO-C18 | A: Water/6.5 mM | From 90% A to 40% A | 1.5 | 40 | 3 |
| | LCMS-2020 | (2.6 μm, 3.0*50 mm) | NH₄HCO₃ | in 1.69 min, to 10% in | | | |
| | | | B: CH₃CN | 0.6 min, held for 0.5 min, | | | |
| | | | | to 90% in 0.03min held | | | |
| | | | | for 0.17 min | | | |
| AE | Shimadzu | Shim-pack Scepter | A: 5 mM NH₄HCO₃ | From 70% A to 30% A | 1.5 | 40 | 3 |
| | LCMS-2020 | C18-120 | in H₂O/CH₃CN | in 1.69 min, to 10% A in | | | |
| | | (3.0 μm, 3.0*33 mm) | (95:5, v:v); | 0.6 min, held for 0.5 min, | | | |
| | | | B: CH₃CN | to 95% A in 0.03 min, | | | |
| | | | | held for 0.17 min | | | |
| AF | Shimadzu | Shim-pack Scepter | A: 5 mM NH₄HCO₃ | From 80% A to 40% A | 1.5 | 40 | 3 |
| | LCMS-2020 | C18-120 | in H₂O/CH₃CN | in 1.69 min, to 10% A in | | | |
| | | (3.0 μm, 3.0*33 mm) | (95:5, v:v); | 0.6 min, held for 0.5 min, | | | |
| | | | B: CH₃CN | to 95% A in 0.03 min, | | | |
| | | | | held for 0.17 min | | | |
| AG | Shimadzu | HALO | A: Water/0.05% TFA | From 80% A to 40% A | 1.5 | 40 | 3 |
| | LCMS-2020 | (2.0 μm, 3.0*30 mm) | B: CH₃CN/0.05% TFA | in 1.69 min, to 5% A in | | | |
| | | | | 0.6 min, held for 0.5 min, | | | |
| | | | | to 95% A in 0.03 min, | | | |
| | | | | held for 0.17 min | | | |
| AH | Shimadzu | HALO 90A C18 | A: Water + 0.05% TFA | From 95% A to 50% A | 1.5 | 40 | 3 |
| | LCMS-2020 | (2.0 μm, 3.0*30 mm) | B: CH₃CN + 0.05% TFA | in 1.69 min, to 5% A in | | | |
| | | | | 0.6 min, held for | | | |
| | | | | 0.5 min, to 95% A in | | | |
| | | | | 0.03 min, held for | | | |
| | | | | 0.17 min | | | |
| AI | Shimadzu | HALO C18 | A: Water/0.1% FA | From 90% A to 5% A | 1.5 | 40 | 10 |
| | LCMS-2020 | (2.7 μm, 100*4.6 mm) | B: CH₃CN/0.1% FA | in 8 min, held for 2 min. | | | |

### Example A

SARS-Cov-2 3CLpro

Protease assays were performed in 384-well low volume polypropylene microtiter plates at ambient temperature. For the duplex assay, 3CLpro was added using a Multidrop Combi (Thermo Scientific; Waltham, MA) and preincubated for 30 mins with small molecules. The reactions were initiated by the addition of the two peptide substrates. The reactions were incubated for 30 mins and quenched by the addition of 0.5% formic acid (final) with subsequent neutralization using 1% sodium bicarbonate (final). Internal standard peptides were added in 20 mM Hepes pH 8.0 for quantitation of the protease products. For SAMDI-MS analysis, 2 μL of each reaction mixture was transferred using a 384-channel automated liquid handler to SAMDI biochip arrays functionalized with a neutravidin-presenting self-assembled monolayer. The SAMDI arrays were incubated for 1 h in a humidified chamber to allow the specific immobilization of the biotinylated peptide substrates, cleaved products and internal standards. The samples were purified by washing the SAMDI arrays with deionized ultrafiltered water and dried with compressed air. A matrix comprising alpha-cyano cinnamic acid in 80% acetonitrile:20% aqueous ammonium citrate was applied in an automated format by dispensing 50 nL to each spot in the array. SAMDI-MS was performed using reflector-positive mode on an AB Sciex TOF-TOF 5800 System (AB Sciex, Framingham, MA) with 400 shots/spot analyzed in a random raster sampling. For data analysis, area under the curves (peaks) (AUCs) for the product and internal standard were calculated using the TOF/TOF Series Explorer (AB Sciex), and the amount of product formed was calculated using the equation (AUC product/AUC internal standard). The amount of product generated was calculated using the ratio of product area under the curve (AUC) divided by the AUC of the internal standard. Negative controls were pre-quenched with 0.5% formic acid final. Assay robustness was determined by Z-Factor. The IC₅₀s were determined by fitting the curves using a four-parameter equation in Graphpad Prism 8.

Table A indicates related IC₅₀ values for the tested compounds where ‘A’ indicates an IC₅₀ <20 nM, ‘B’ indicates an IC₅₀ of ≥20 nM and <200 nM, ‘C’ indicates an IC₅₀ ≥200 nM and <2000 nM, ‘D’ indicates an IC₅₀ ≥2000 nM and <20000 nM and ‘E’ indicates an IC₅₀ ≥20000 nM and <100000 nM. As shown by the data in Table A, compounds described herein (including pharmaceutically acceptable salts thereof) can effectively inhibit and be used to treat a coronavirus and rhinovirus.

TABLE A

| Compound | SARS-Cov-2 |
|---|---|
| 1 | A |
| 2 | A |
| 3 | B |
| 4 | B |
| 5 | C |
| 6 | A |
| 7 | B |
| 8 | B |
| 9 | E |
| 10 | C |
| 11 | B |
| 12 | B |
| 13 | A |
| 14 | D |
| 15 | A |
| 16 | B |
| 17 | B |
| 18 | B |
| 19 | B |
| 20 | B |
| 21 | A |
| 22 | B |
| 23 | A |
| 24 | A |
| 25 | B |
| 26 | B |
| 27 | B |
| 28 | A |
| 29 | B |
| 30 | A |
| 31 | B |
| 32 | A |
| 33 | A |
| 34 | A |
| 35 | B |
| 36 | A |
| 37 | A |
| 38 | A |
| 39 | A |
| 40 | C |
| 41 | C |
| 42 | B |
| 43 | B |
| 44 | B |
| 45 | B |
| 46 | B |
| 47 | B |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | B |
| 69 | A |
| 70 | A |
| 71 | A |
| 72 | A |
| 73 | B |
| 74 | B |
| 80 | C |
| 81 | A |
| 82 | B |
| 83 | B |
| 84 | A |
| 85 | A |
| 86 | A |
| 87 | A |
| 88 | A |
| 89 | A |
| 90 | A |
| 91 | A |
| 92 | C |
| 93 | A |
| 94 | B |
| 95 | A |
| 96 | A |
| 97 | A |
| 98 | A |
| 99 | A |
| 100 | B |
| 101 | B |
| 102 | A |
| 103 | A |
| 104 | B |
| 105 | B |
| 106 | A |
| 107 | A |
| 108 | A |
| 109 | A |
| 110 | A |
| 111 | A |
| 112 | A |
| 113 | B |
| 114 | A |
| 115 | A |
| 116 | A |
| 117 | C |
| 118 | C |
| 119 | A |
| 120 | A |
| 121 | A |
| 122 | A |
| 123 | >2 μM |
| 124 | A |
| 125 | A |
| 126 | A |
| 127 | ND |
| 128 | C |
| 129 | C |
| 130 | B |
| 131 | B |
| 132 | B |
| 133 | A |
| 134 | A |
| 135 | A |
| 136 | A |
| 137 | B |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | B |
| 154 | C |
| 155 | B |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | B |
| 172 | A |
| 173 | C |
| 174 | A |
| 175 | A |
| 176 | A |
| 177 | A |
| 178 | A |
| 179 | A |
| 180 | C |
| 181 | A |
| 182 | A |
| 183 | A |
| 184 | A |
| 185 | A |
| 186 | A |
| 187 | A |
| 188 | A |
| 189 | B |
| 190 | A |
| 191 | A |
| 192 | B |
| 193 | B |
| 194 | A |
| 195 | A |
| 196 | A |
| 197 | A |
| 198 | A |
| 199 | A |
| 200 | A |
| 201 | B |
| 202 | A |
| 203 | A |
| 204 | A |
| 205 | A |
| 206 | A |
| 207 | B |
| 208 | B |
| 209 | A |
| 210 | C |
| 211 | B |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | B |
| 224 | A |
| 225 | B |
| 226 | C |
| 227 | A |
| 228 | B |
| 229 | A |
| 230 | A |
| 231 | >2 μM |
| 232 | C |
| 233 | A |
| 234 | A |
| 235 | A |
| 236 | A |
| 237 | C |
| 238 | A |
| 239 | A |
| 240 | A |

### Example B

Coronavirus Assay

OC43 Coronavirus Assay in HeLa cells

The human beta-coronavirus OC43 was purchased from ATCC (Manassas, VA) and propagated using HCT-8 human colorectal epithelial cells (ATCC). HeLa human cervical epithelial cells (ATCC) were used as susceptible host cell lines and were cultured using EMEM media, supplemented with 10% fetal bovine serum (FBS), 1% (v/v) penicillin/streptomycin (P/S), 1% (v/v) HEPES and 1% (v/v) Cellgro Glutagro™ supplement (all Corning, Manassas, VA) at 37° C. For the OC43 antiviral assay, 1.5×10⁴ HeLa cells per well were plated in 100 μL complete media in white 96-well plates with clear bottoms at 37° C. for up to 24 h to facilitate attachment and allow cells to recover from seeding stresses. Next day, the cell culture medium was removed. Serially diluted compounds in 100 μL assay media (EMEM, 2% FBS, 1% P/S, 1% Cellgro Glutagro™ supplement, 1% HEPES) were added to the cells and incubated for 4 h at 37° C. in a humidified 5% CO₂ incubator. 100 μL of OC43 virus stock was diluted to a concentration known to produce optimal cytopathic effect, inducing 80-90% reduction in cell viability. 96-well plates were incubated for 6 days at 33° C.; each plate contains uninfected control wells as well as virus-infected wells that were not treated with compound. Cytotoxicity plates without the addition of OC43 virus were carried out in parallel. At the end of the incubation period, 100 μL cell culture supernatant was replaced with 100 μL cell-titer-glo reagent (Promega, Madison, WI) and incubated for at least 10 min at rt prior to measuring luminescence. Luminescence was measured on a Perkin Elmer (Waltham, MA) Envision plate reader. Antiviral % inhibition was calculated as follows: [(Compound treated cells infected sample)−(no compound infected control)]/[(Uninfected control)−(no compound infected control)]*100; Using GraphPad (San Diego, CA) prism software version 8.3.1, the antiviral dose-response plot was generated as a sigmoidal fit, log(inhibitor) vs response-variable slope (four parameters) model and the EC₅₀ was calculated which is the predicted compound concentration corresponding to a 50% inhibition of the viral cytopathic effect.

Table B indicates related EC₅₀ and CC₅₀ values for the tested compounds ‘A’ indicates an EC₅₀ <100 nM, ‘B’ indicates an EC₅₀ of ≥100 nM and <1000 nM, ‘C’ indicates an EC₅₀ ≥1000 nM and <10000 nM and ‘D’ indicates an EC₅₀ ≥10000 nM and <100000 nM. For CC₅₀, the values are reported in micromolar (m), ‘A’ indicates a CC₅₀ ≥10000 nM and ‘B’ indicates a CC₅₀ ≥1 μM and <10 μM.

TABLE B

| Compound | EC₅₀ | CC₅₀ |
|---|---|---|
| 1 | A | A |
| 2 | B | A |
| 3 | >1 μM | A |
| 4 | >1 μM | A |
| 5 | >1 μM | A |
| 6 | B | A |
| 7 | ND | |
| 8 | C | A |
| 9 | ND | |
| 10 | ND | |
| 11 | C | A |
| 12 | C | A |
| 13 | B | A |
| 14 | ND | |
| 15 | A | A |
| 16 | B | A |
| 17 | C | A |
| 18 | C | A |
| 19 | B | A |
| 20 | C | A |
| 21 | B | A |
| 22 | C | A |
| 23 | B | A |
| 24 | C | A |
| 25 | B | A |
| 26 | B | A |
| 27 | C | A |
| 28 | A | A |
| 29 | C | A |
| 30 | B | A |
| 31 | >10 μM | A |
| 32 | C | A |
| 33 | B | A |
| 34 | B | A |
| 35 | C | A |
| 36 | A | A |
| 37 | A | A |
| 38 | ND | |
| 39 | ND | |
| 40 | ND | |
| 41 | ND | |
| 42 | >10 μM | A |
| 43 | C | A |
| 44 | B | A |
| 45 | B | A |
| 46 | >10 μM | A |
| 47 | C | A |
| 48 | B | A |
| 49 | B | A |
| 50 | A | A |
| 51 | B | A |
| 52 | A | A |
| 53 | B | A |
| 54 | A | A |
| 55 | A | A |
| 56 | A | A |
| 57 | A | A |
| 58 | B | A |
| 59 | A | A |
| 60 | A | A |
| 61 | C | A |
| 62 | B | A |
| 63 | B | A |
| 64 | A | A |
| 65 | B | A |
| 66 | A | A |
| 67 | A | A |
| 69 | >10 μM | A |
| 70 | >10 μM | A |
| 71 | B | A |
| 72 | B | A |
| 73 | B | A |
| 74 | >10 μM | A |
| 75 | >10 μM | A |
| 76 | A | A |
| 77 | A | A |
| 78 | B | A |
| 79 | A | A |
| 80 | A | A |
| 81 | A | A |
| 82 | B | A |
| 83 | B | A |
| 84 | C | A |
| 85 | B | A |
| 86 | B | A |
| 87 | B | A |
| 88 | B | A |
| 89 | A | A |
| 90 | A | A |
| 91 | A | A |
| 92 | A | A |
| 93 | A | A |
| 94 | >10 μM | A |
| 95 | C | A |
| 96 | C | A |
| 97 | A | A |
| 98 | B | A |
| 99 | B | A |
| 100 | A | A |
| 101 | A | A |
| 102 | C | A |
| 103 | C | A |
| 104 | A | A |
| 105 | ND | ND |
| 106 | B | A |
| 107 | C | A |
| 108 | B | A |
| 109 | ND | ND |
| 110 | A | A |
| 111 | B | A |
| 112 | A | A |
| 113 | B | A |
| 114 | B | A |
| 115 | B | A |
| 116 | B | A |
| 117 | >10 μM | A |
| 118 | >10 μM | A |
| 119 | B | A |
| 120 | B | A |
| 121 | A | A |
| 122 | A | A |
| 123 | ND | ND |
| 124 | A | A |
| 125 | A | A |
| 126 | A | A |
| 127 | ND | ND |
| 128 | >10 | A |
| 129 | >10 | A |
| 130 | B | A |
| 131 | C | A |
| 132 | C | A |
| 133 | B | A |
| 134 | >10 μM | ND |
| 135 | A | A |
| 136 | B | A |
| 137 | C | A |
| 138 | B | A |
| 139 | A | A |
| 140 | A | A |
| 141 | A | A |
| 142 | A | A |
| 143 | A | A |
| 144 | A | A |
| 145 | ND | ND |
| 146 | B | A |
| 147 | A | A |
| 148 | ND | ND |
| 149 | A | A |
| 150 | A | A |
| 151 | ND | ND |
| 152 | ND | ND |
| 153 | ND | ND |
| 154 | C | A |
| 155 | B | A |
| 156 | A | A |
| 157 | A | A |
| 158 | A | A |
| 159 | B | A |
| 160 | A | A |
| 161 | ND | ND |
| 162 | ND | ND |
| 163 | A | A |
| 164 | A | A |
| 165 | A | A |
| 166 | A | A |
| 167 | A | A |
| 168 | A | A |
| 169 | A | A |
| 170 | A | A |
| 171 | C | A |
| 172 | B | A |
| 173 | C | A |
| 174 | A | A |
| 175 | A | A |
| 176 | A | A |
| 177 | B | A |
| 178 | A | A |
| 179 | A | A |
| 180 | ND | ND |
| 181 | C | A |
| 182 | A | A |
| 183 | A | A |
| 184 | A | A |
| 185 | A | A |
| 186 | A | A |
| 187 | A | A |
| 188 | A | A |
| 189 | B | A |
| 190 | A | A |
| 191 | A | A |
| 192 | B | A |
| 193 | B | A |
| 194 | A | A |
| 195 | B | A |
| 196 | A | A |
| 197 | A | A |
| 198 | A | A |
| 199 | A | A |
| 200 | B | A |
| 201 | B | A |
| 202 | B | A |
| 203 | A | A |
| 204 | A | A |
| 205 | A | A |
| 206 | A | A |
| 207 | A | A |
| 208 | B | A |
| 209 | A | A |
| 210 | ND | ND |
| 211 | ND | ND |
| 212 | ND | ND |
| 213 | A | A |
| 214 | A | A |
| 215 | A | A |
| 216 | ND | ND |
| 217 | ND | ND |
| 218 | ND | ND |
| 219 | A | A |
| 220 | A | A |
| 221 | >10 μM | A |
| 222 | A | A |
| 223 | B | A |
| 224 | A | A |
| 225 | B | A |
| 226 | C | A |
| 227 | A | A |
| 228 | B | A |
| 229 | A | A |
| 230 | B | A |
| 231 | >10 μM | A |
| 232 | >10 μM | A |
| 233 | B | A |

### B.1.1.7 Infection Model in A549-Dual ACE2 TMPRSS2 Cells

The A549-dual_ACE2_TMPRSS2 cells (InvivoGen Cat #a-49-cov2r) were propagated in the growth medium which was prepared by supplementing DMEM (gibco cat no 41965-039) with 10% v/v heat-inactivated FCS and 10 μg/mL blasticidin (InvivoGen ant-b1-05), 100 μg/mL hygromycin (InvivoGen ant-hg-1), 0.5 μg/mL puromycin (InvivoGen ant-pr-1) and 100 μg/mL zeocin (InvivoGen ant-zn-05) in a humidified 5% CO₂ incubator at 37° C. The assay medium was prepared by supplementing DMEM (gibco cat no 41965-039) with 2% v/v heat-inactivated FCS.

The virus isolate used is from the B.1.1.7 lineage (derived from hCoV-19/Belgium/rega-12211513/2020; EPI_ISL_791333,2020-12-21; see Abdelnabi et al., “Comparing infectivity and virulence of emerging SARS-CoV-2 variants in Syrian hamsters” EBioMedicine (2021) June; 68:103403. doi: 10.1016/j.ebiom.2021.103403).

For antiviral testing, cells were seeded in 96-well plates (Falcon) at a density of 15,000 cells per well in assay medium. After overnight growth, cells were treated with the indicated compound concentrations and infected with a MOI of 0.001 TCID50/cell (final volume 200 μL/well in assay medium). On day 4p.i. differences in cell viability caused by virus-induced CPE or by compound-specific side effects are analyzed using MTS as described previously (PMID: 22575574).

For toxicity testing, the same experimental setup was used except that assay medium without virus was added to the cells and that an additional control of well without cells was added to the plate.

Table C indicates related EC₅₀ and CC₅₀ values for the tested compounds ‘A’ indicates an EC₅₀ <100 nM, ‘B’ indicates an EC₅₀ of ≥100 nM and <1000 nM, ‘C’ indicates an EC₅₀ ≥1000 nM and <10000 nM. For CC₅₀, the values are reported in micromolar (μM). ‘A’ indicates a CC₅₀ ≥1000 nM. ‘B’ indicates a CC₅₀ ≥100 nM and <1000 nM.

TABLE C

| | A549-dual_ACE2_TMPRSS2 | A549-dual_ACE2_TMPRSS2 |
|---|---|---|
| Compound | (EC₅₀) | (CC₅₀) |
| 1 | A | A |
| 2 | B | A |
| 3 | C | A |
| 4 | C | A |
| 5 | C | A |
| 6 | B | A |
| 7 | C | A |
| 8 | C | A |
| 9 | C | A |
| 10 | C | A |
| 11 | B | A |
| 12 | C | A |
| 13 | B | A |
| 14a | B | A |
| 14b | >1 μM | A |
| 15 | A | A |
| 16 | >1 μM | A |
| 17 | B | A |
| 18 | >1 μM | A |
| 19 | B | A |
| 20 | B | A |
| 21 | B | A |
| 22 | >1 μM | A |
| 23 | B | A |
| 24 | B | A |
| 25 | B | A |
| 26 | >1 μM | A |
| 27 | >1 μM | A |
| 28 | >1 μM | A |
| 29 | B | A |
| 30 | B | A |
| 31 | >1 μM | A |
| 32 | B | A |
| 33 | A | A |
| 34 | B | A |
| 35 | B | A |
| 36 | B | A |
| 37 | B | A |
| 38 | B | A |
| 39 | A | A |
| 40 | >1 μM | A |
| 41 | >1 μM | A |
| 42 | B | A |
| 43 | B | A |
| 44 | >1 μM | A |
| 45 | >1 μM | A |
| 46 | >1 μM | A |
| 47 | B | A |
| 48 | B | A |
| 49 | B | A |
| 50 | A | A |
| 51 | B | A |
| 52 | A | A |
| 53 | B | A |
| 54 | B | A |
| 55 | A | A |
| 56 | A | A |
| 57 | A | A |
| 58 | B | A |
| 59 | A | A |
| 60 | A | A |
| 61 | B | A |
| 62 | B | A |
| 63 | B | A |
| 64 | B | A |
| 65 | B | A |
| 66 | A | A |
| 67 | B | A |
| 68 | >1 μM | A |
| 69 | B | A |
| 70 | B | A |
| 71 | B | A |
| 72 | B | A |
| 73 | B | A |
| 74 | B | A |
| 75 | A | A |
| 76 | A | A |
| 77 | A | A |
| 78 | B | A |
| 79 | B | A |
| 81 | A | A |
| 82 | >1 uM | A |
| 83 | A | A |
| 84 | A | A |
| 85 | A | A |
| 86 | A | A |
| 87 | A | A |
| 88 | A | A |
| 89 | A | A |
| 90 | A | A |
| 92 | A | A |
| 93 | A | A |
| 94 | B | A |
| 95 | A | A |
| 96 | B | A |
| 97 | B | A |
| 98 | A | A |
| 99 | A | A |
| 100 | B | A |
| 101 | B | A |
| 102 | A | A |
| 103 | B | A |
| 104 | B | A |
| 105 | B | A |
| 106 | B | A |
| 107 | B | A |
| 108 | A | A |
| 109 | B | A |
| 110 | >1 μM | A |
| 111 | B | A |
| 112 | B | A |
| 113 | >1 μM | A |
| 114 | B | A |
| 115 | B | A |
| 116 | B | A |
| 117 | >1 μM | A |
| 118 | >1 μM | A |
| 119 | >1 μM | A |
| 120 | B | A |
| 121 | B | A |
| 122 | A | A |
| 123 | ND | ND |
| 124 | B | A |
| 125 | A | A |
| 126 | B | A |
| 127 | ND | ND |
| 128 | B | A |
| 129 | B | A |
| 130 | B | A |
| 131 | >1 μM | A |
| 132 | B | A |
| 133 | B | A |
| 134 | >1 μM | A |
| 135 | B | A |
| 136 | B | A |
| 137 | B | A |
| 138 | B | A |
| 139 | B | A |
| 140 | A | A |
| 141 | B | A |
| 142 | A | A |
| 143 | B | A |
| 144 | B | A |
| 145 | A | A |
| 146 | B | A |
| 147 | A | A |
| 148 | >1 μM | A |
| 149 | A | A |
| 150 | A | A |
| 151 | B | A |
| 152 | A | A |
| 153 | B | A |
| 154 | >1 μM | A |
| 155 | B | A |
| 156 | B | A |
| 157 | A | A |
| 158 | A | A |
| 159 | A | A |
| 160 | A | A |
| 161 | A | A |
| 162 | A | A |
| 163 | B | A |
| 164 | A | A |
| 165 | A | A |
| 166 | B | A |
| 167 | B | A |
| 168 | A | A |
| 169 | B | A |
| 170 | A | A |
| 171 | >1 μM | A |
| 172 | B | A |
| 173 | >1 μM | A |
| 174 | A | A |
| 175 | B | A |
| 176 | A | A |
| 177 | B | A |
| 178 | B | A |
| 179 | B | A |
| 180 | >1 μM | A |
| 181 | B | A |
| 182 | A | A |
| 183 | B | A |
| 184 | A | A |
| 185 | B | A |
| 186 | B | A |
| 187 | B | A |
| 188 | A | A |
| 189 | B | A |
| 190 | B | A |
| 191 | B | A |
| 192 | B | A |
| 193 | B | A |
| 194 | A | A |
| 195 | B | A |
| 196 | B | A |
| 197 | B | A |
| 198 | B | A |
| 199 | B | A |
| 200 | A | A |
| 201 | B | A |
| 202 | >1 μM | A |
| 203 | A | A |
| 204 | A | A |
| 205 | A | A |
| 206 | A | A |
| 207 | A | A |
| 208 | B | A |
| 209 | B | A |
| 210 | >1 μM | A |
| 211 | B | A |
| 212 | B | A |
| 213 | B | A |
| 214 | B | A |
| 215 | A | A |
| 216 | B | A |
| 217 | B | A |
| 218 | A | A |
| 219 | B | A |
| 220 | B | A |
| 221 | ND | ND |
| 222 | A | A |
| 223 | ND | ND |
| 224 | ND | ND |
| 225 | B | A |
| 226 | >1 μM | A |
| 227 | B | A |
| 228 | B | A |
| 229 | B | A |
| 233 | A | A |

Tables A, B and C demonstrate that compounds described herein (including pharmaceutically acceptable salts thereof) can effectively inhibit and treat a coronavirus.

### Example C

Picornavirus & Norovirus Assays

Compounds of Formula (I), including pharmaceutically acceptable salts thereof, are tested following a protocol similar to the protocol described in one of the following articles: Kim et al., Journal of Virology (2012) 86(21):11754-11762, Zhang et al, JACS (2020) (https://dx.doi.org/10.1021/acs.jmedchem.9b01828), and U.S. Pat. No. 9,603,864.

The protocols of Kim et al., and Zhang et al., can be used to test for activity against a picornavirus and norovirus.

### Example D

For the cathepsin L assay, 10 pM of human cathepsin L (R&D Systems; Minneapolis, MN) are preincubated for 30 mins with test compounds. Reactions are initiated by the addition of a peptide substrate Z—FR-AMC (final concentration 2 PM, Anaspec; Fremont, CA). Fluorescence is measured at 2-minute intervals for 30 mins using a 355/460 excitation/emission filter module on an Envision plate reader (Perkin Elmer; Waltham, MA). The IC₅₀ values are calculated for each assay by fitting the curves using a four-parameter equation in GraphPad Prism.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

1. A compound of Formula (I), or a pharmaceutically acceptable salt thereof, having the structure: 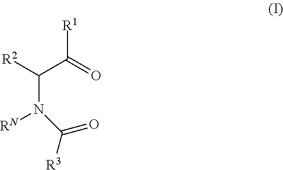
wherein:
R^{N} is hydrogen, deuterium or an unsubstituted or a substituted C₁₋₆ alkyl;
R¹ is 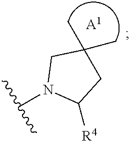
wherein Ring A¹ is a 5-7 membered monocyclic heterocyclyl:
i) includes NR^{5a} in the ring;
ii) is substituted with a first ═O on a carbon of the ring;
iii) optionally includes 1-3 heteroatoms selected from the group consisting of O, S, S(═O)₂, N and NR^{5b} in the ring of Ring A¹;
iv) is optionally substituted with one or more moieties selected from the group consisting of a second ═O on a ring carbon, halogen, hydroxy, an unsubstituted C₁₋₆ alkyl, an unsubstituted —O(C₁₋₆ alkyl), an unsubstituted or a substituted phenoxy, an unsubstituted or a substituted C₃₋₆ cycloalkyl, an unsubstituted or a substituted phenyl and an unsubstituted or a substituted benzyl;
v) is optionally fused to an unsubstituted or a substituted phenyl, an unsubstituted or a substituted monocyclic heteroaryl, an unsubstituted or a substituted monocyclic heterocyclyl, an unsubstituted or a substituted bicyclic heteroaryl, an unsubstituted or a substituted bicyclic heterocyclyl, an unsubstituted or a substituted monocyclic cycloalkenyl or an unsubstituted or a substituted bicyclic cycloalkenyl; and
vi) provided that when R¹ is 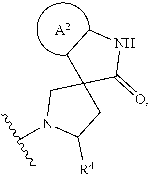
then Ring A² can be an unsubstituted or a substituted monocyclic heterocyclyl, an unsubstituted or a substituted monocyclic cycloalkenyl or an unsubstituted or a substituted bicyclic cycloalkenyl;
R² is hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₁₀ cycloalkyl, an unsubstituted or a substituted C₃₋₁₀ cycloalkenyl, an unsubstituted or a substituted aryl, an unsubstituted or a substituted aryl(alkyl), an unsubstituted or a substituted heteroaryl, an unsubstituted or a substituted heteroaryl(alkyl), an unsubstituted or a substituted heterocyclyl or an unsubstituted or a substituted heterocyclyl(alkyl);
R³ is 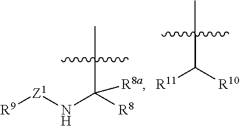
Z¹ is —C(═O)— or —S(═O)₂—;
R⁴ is selected from the group consisting of cyano, an unsubstituted or a substituted C₂₋₅ alkynyl, an unsubstituted or a substituted acyl, an unsubstituted or a substituted ketoamide, —C(═O)NH₂, —CH(OH)—(S(═O)₂—OH), —CH(OH)—(S(═O)₂—O—), —CH(OH)((P═O)(OR⁶)₂) and —C(═O)CH₂—O—((P═O)(OR⁷)₂);
R^{5a} is selected from the group consisting of hydrogen, an unsubstituted or a substituted C₁₋₄ alkyl, an unsubstituted or a substituted C₂₋₄ alkenyl and an unsubstituted or a substituted C₃₋₆ cycloalkyl;
R^{5b} is selected from the group consisting of hydrogen, an unsubstituted or a substituted C₁₋₄ alkyl, an unsubstituted or a substituted C₂₋₄ alkenyl and an unsubstituted or a substituted C₃₋₆ cycloalkyl;
each R⁶ and each R⁷ are independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl);
R⁸ and R¹⁰ are independently selected from the group consisting of an unsubstituted or a substituted C₁₋₆ alkyl, an unsubstituted or a substituted C₂₋₆ alkenyl, an unsubstituted or a substituted C₂₋₆ alkynyl, an unsubstituted or a substituted monocyclic C₃₋₆ cycloalkyl, an unsubstituted or a substituted bicyclic C₅₋₈ cycloalkyl and an unsubstituted or a substituted monocyclic 4- to 6-membered heterocyclyl,
wherein when the C₁₋₆ alkyl is substituted, the C₁₋₆ alkyl is substituted 1, 2, 3 or 4 times with a substituent independently selected from the group consisting of halogen, cyano, —NH₂, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic 5- or 6-membered heteroaryl, an unsubstituted or a substituted monocyclic 4-6 membered heterocyclyl, an unsubstituted C₁₋₄ alkoxy, an unsubstituted or a substituted phenoxy, an unsubstituted or a substituted —O—(CH₂)-phenyl and an unsubstituted C₁₋₄ haloalkoxy, or the C₁₋₆ alkyl is substituted 1 to 13 times with deuterium;
wherein when the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted, the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted 1, 2, 3 or 4 times with a substituent independently selected from the group consisting of halogen, an unsubstituted C₁₋₄ alkyl, an unsubstituted C₂₋₄ alkenyl, an unsubstituted C₂₋₄ alkynyl, an unsubstituted C₁₋₄ haloalkyl, an unsubstituted or a substituted monocyclic C₃₋₆ cycloalkyl and an unsubstituted C₁₋₄ alkoxy; and
R^{8a} is hydrogen or an unsubstituted C₁₋₄ alkyl; or
R⁸ and R^{8a} are taken together to form an unsubstituted monocyclic C₃₋₆ cycloalkyl or a halogen-substituted monocyclic C₃₋₆ cycloalkyl;
R⁹ is selected from the group consisting of an unsubstituted or a substituted C₁₋₆ alkyl, an unsubstituted or a substituted C₁₋₆ haloalkyl, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic heteroaryl, an unsubstituted or a substituted monocyclic heterocyclyl, an unsubstituted or a substituted alkoxy and —NR¹⁷R¹⁸, wherein the substituted C₁₋₆ alkyl is substituted 1 or 2 times with a substituent selected from hydroxy and an unsubstituted C₁₋₄ alkoxy, wherein the substituted monocyclic C₃₋₆ cycloalkyl is substituted 1, 2, 3 or 4 times with a substituent independently selected from the group consisting of halogen, an unsubstituted C₁₋₄ alkyl, an unsubstituted C₁₋₄ alkoxy, an unsubstituted C₁₋₄ haloalkyl and an unsubstituted monocyclic C₃₋₆ cycloalkyl, and wherein the substituted C₁₋₆ haloalkyl is substituted 1 or 2 times with an unsubstituted C₁₋₄ alkoxy;
R¹¹ is an unsubstituted or a substituted monocyclic 4- to 6-membered heterocyclyl, —(NH)ₘ-(an unsubstituted or a substituted 5- to 10-membered heteroaryl), —O-(an unsubstituted or a substituted C₁₋₆ alkyl), —O-(an unsubstituted or a substituted C₃₋₈ cycloalkyl) or —O—(C₁₋₄ alkyl)−(an unsubstituted or a substituted C₃₋₈ cycloalkyl), wherein m is 1;
R¹² is an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 12-membered monocyclic heterocyclyl, an unsubstituted or a substituted 5- to 12-membered bicyclic heterocyclyl, an unsubstituted or a substituted aryl(alkyl), an unsubstituted or a substituted heteroaryl(alkyl), an unsubstituted or a substituted heterocyclyl(alkyl), an unsubstituted or a substituted C-carboxy, —OR¹³—NR¹⁴R¹⁵ or —C(═O)—NR^{16A}R^{16B};
R¹³ is an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl);
R¹⁴ are R¹⁵ are independently selected from the group consisting of hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl);
R^{16A} is hydrogen or an unsubstituted C₁₋₃ alkyl;
R^{16B} is an unsubstituted or a substituted aryl, an unsubstituted or a substituted heteroaryl or an unsubstituted or a substituted 3- to 8-membered monocyclic heterocyclyl; and
R¹⁷ and R¹⁸ are independently selected from the group consisting of hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl); or
R¹⁷ and R¹⁸ are taken together along with the nitrogen to which they are connected to form an unsubstituted or a substituted 3-8 membered heterocyclyl.

2. The compound of claim 1, wherein R¹ is 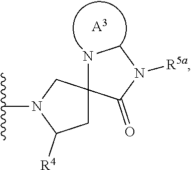
wherein Ring A³ is absent, an unsubstituted or a substituted monocyclic heteroaryl, an unsubstituted or a substituted monocyclic heterocyclyl, an unsubstituted or a substituted bicyclic heteroaryl or an unsubstituted or a substituted bicyclic heterocyclyl; wherein R¹ is 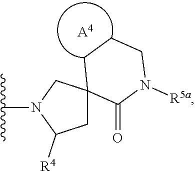
wherein Ring A⁴ is absent, an unsubstituted or a substituted phenyl, an unsubstituted or a substituted monocyclic cycloalkenyl, an unsubstituted or a substituted monocyclic heteroaryl, an unsubstituted or a substituted monocyclic heterocyclyl, an unsubstituted or a substituted bicyclic cycloalkenyl, an unsubstituted or a substituted bicyclic heteroaryl or an unsubstituted or a substituted bicyclic heterocyclyl; wherein R¹ is 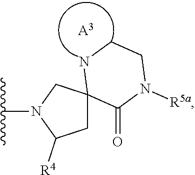
wherein Ring A³ is absent, an unsubstituted or a substituted monocyclic heteroaryl, an unsubstituted or a substituted monocyclic heterocyclyl, an unsubstituted or a substituted bicyclic heteroaryl or an unsubstituted or a substituted bicyclic heterocyclyl; wherein R¹ is 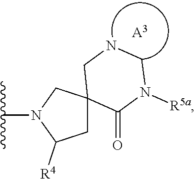
wherein Ring A³ is absent, an unsubstituted or a substituted monocyclic heteroaryl, an unsubstituted or a substituted monocyclic heterocyclyl, an unsubstituted or a substituted bicyclic heteroaryl or an unsubstituted or a substituted bicyclic heterocyclyl; wherein R¹ is 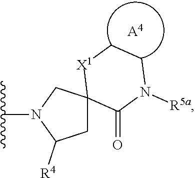
wherein Ring A⁴ is absent, an unsubstituted or a substituted phenyl, an unsubstituted or a substituted monocyclic cycloalkenyl, an unsubstituted or a substituted monocyclic heteroaryl, an unsubstituted or a substituted monocyclic heterocyclyl, an unsubstituted or a substituted bicyclic cycloalkenyl, an unsubstituted or a substituted bicyclic heteroaryl or an unsubstituted or a substituted bicyclic heterocyclyl; and X¹ is —CR^{Y1}R^{Y2}—, —CR^{Y3}R^{Y4}CR^{Y5}R^{Y6}—, —O—, —S—, S(═O)₂, NR^{5b} or —OCH₂—, wherein R^{Y1}, R^{Y2}, R^{Y3}, R^{Y4}, R^{Y5} and R^{Y6} are independently selected from hydrogen, halogen, hydroxy and an unsubstituted C₁₋₆ alkyl; wherein R¹ is 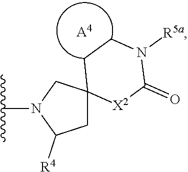
Ring A⁴ is absent, an unsubstituted or a substituted phenyl, an unsubstituted or a substituted monocyclic cycloalkenyl, an unsubstituted or a substituted monocyclic heteroaryl, an unsubstituted or a substituted monocyclic heterocyclyl, an unsubstituted or a substituted bicyclic cycloalkenyl, an unsubstituted or a substituted bicyclic heteroaryl or an unsubstituted or a substituted bicyclic heterocyclyl; and X² is —CH₂—, —CH₂CH₂—, —O—, —S—, —S(═O)₂—, NR^{5b} or —OCH₂—; wherein R¹ is 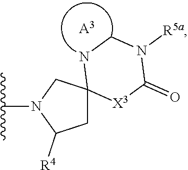
Ring A³ is absent, an unsubstituted or a substituted monocyclic heteroaryl, an unsubstituted or a substituted monocyclic heterocyclyl, an unsubstituted or a substituted bicyclic heteroaryl or an unsubstituted or a substituted bicyclic heterocyclyl; and X³ is —CH₂—, —CH₂CH₂—, —O—, —S—, —S(═O)₂—, NR^{5b} or —OCH₂—; wherein R¹ is 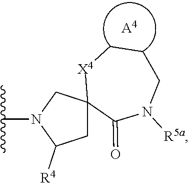
Ring A⁴ is absent, an unsubstituted or a substituted phenyl, an unsubstituted or a substituted monocyclic cycloalkenyl, an unsubstituted or a substituted monocyclic heteroaryl, an unsubstituted or a substituted monocyclic heterocyclyl, an unsubstituted or a substituted bicyclic cycloalkenyl, an unsubstituted or a substituted bicyclic heteroaryl or an unsubstituted or a substituted bicyclic heterocyclyl; and X⁴ is —CH₂—, —O—, —S—, —S(═O)₂— or NR^{5b}; wherein R¹ is 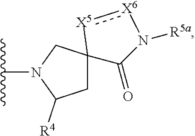
wherein [symbol] is a single bond; X⁵ is —CR^{Z1a}R^{Z1b}—, —(CR^{Z2a}R^{Z2b})(CR^{Z3a}R^{Z3b})—, —O—, —S—, —O(CR^{Z4a}R^{Z4b})—, —S(CR^{Z5a}R^{Z5b})—, —O(CR^{Z6a}R^{Z6b})(CR^{Z7a}R^{Z7b})—, —S(CR^{Z8a}R^{Z8b})(CR^{Z9a}R^{Z9b})— or NR^{5b}; X⁶ is —CR^{Z10a}R^{Z10b}—, —C(═O)— or NR^{5b}; provided that X⁵ and X⁶ cannot be both NR^{5b}, R^{Z1a}, R^{Z1b}, R^{Z4a}, R^{Z4b}, R^{Z5a}, R^{Z5b}, R^{Z6a}, R^{Z6b}, R^{Z7a}, R^{Z7b}, R^{Z8a}, R^{Z8b}, R^{Z9a} and R^{z9b} are independently selected from the group consisting of hydrogen, halogen, hydroxy, an unsubstituted C₁₋₆ alkyl, an unsubstituted —O(C₁₋₆ alkyl), an unsubstituted or a substituted C₃₋₆ cycloalkyl, an unsubstituted or a substituted phenyl, an unsubstituted or a substituted phenoxy and an unsubstituted or a substituted benzyl; R^{Z10a} and R^{Z10b} can be independently selected from hydrogen, halogen, hydroxy, an unsubstituted C₁₋₆ alkyl, an unsubstituted —O(C₁₋₆ alkyl), an unsubstituted or a substituted C₃₋₆ cycloalkyl (such as an unsubstituted or a substituted monocyclic C₃₋₆ cycloalkyl), an unsubstituted or a substituted phenyl, an unsubstituted or a substituted phenoxy, and an unsubstituted or a substituted benzyl; or R^{Z10a} and R^{Z10b} can be taken together along with the carbon to which R^{Z10a} and R^{Z10b} are attached to form an unsubstituted or a substituted spiro-connected C₃₋₆ cycloalkyl; and R^{Z2a}, R^{Z2b}, R^{Z3a} and R^{Z3b} are independently selected from the group consisting of hydrogen, halogen, hydroxy, an unsubstituted C₁₋₆ alkyl, an unsubstituted or a substituted C₃₋₆ cycloalkyl, an unsubstituted or a substituted phenyl and an unsubstituted or a substituted benzyl, or R^{Z2a} and R^{Z3a} are taken together along with the carbons to which each is attached to form an unsubstituted or a substituted monocyclic cycloalkenyl or an unsubstituted or a substituted bicyclic cycloalkenyl; or wherein ----- is a double bond; X⁵ is CR^{Z11} or CR^{Z12a}R^{Z12b}CR^{Z13}; X⁶ is N or CR^{Z14}; and R^{Z11}, R^{Z12a}, R^{Z12b}, R^{Z13} and R^{Z14} are independently selected from the group consisting of hydrogen, halogen, hydroxy, an unsubstituted C₁₋₆ alkyl, an unsubstituted or a substituted C₃₋₆ cycloalkyl, an unsubstituted or a substituted phenyl and an unsubstituted or a substituted benzyl; or wherein R¹ is 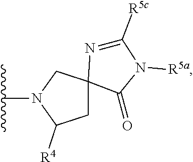
wherein R^{5C} is selected from the group consisting of hydrogen, halogen, hydroxy, an unsubstituted C₁₋₆ alkyl, an unsubstituted or a substituted C₃₋₆ cycloalkyl, an unsubstituted or a substituted phenyl and an unsubstituted or a substituted benzyl.

3.-24. (canceled)

25. The compound of claim 1, wherein R¹ is selected from the group consisting of: 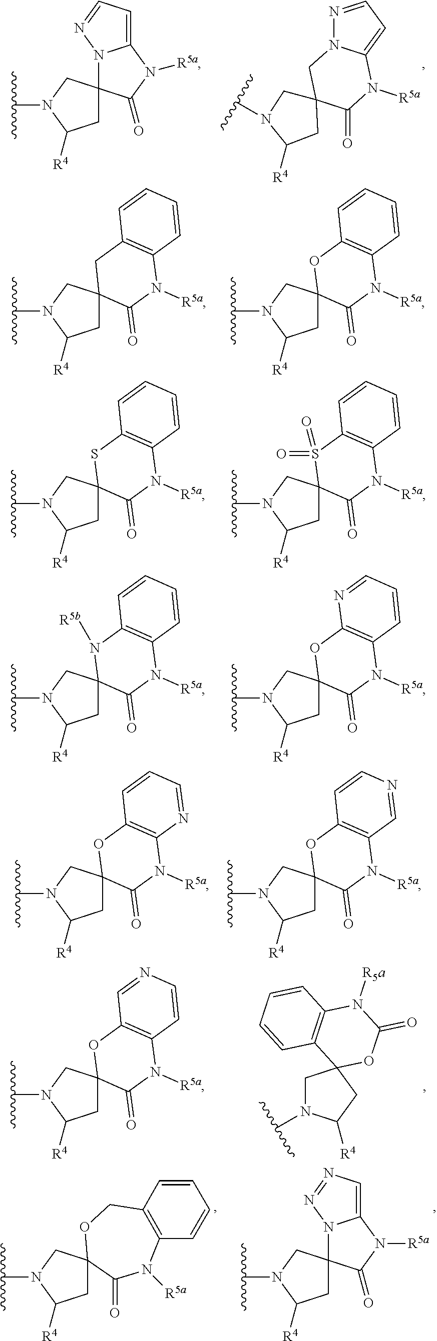 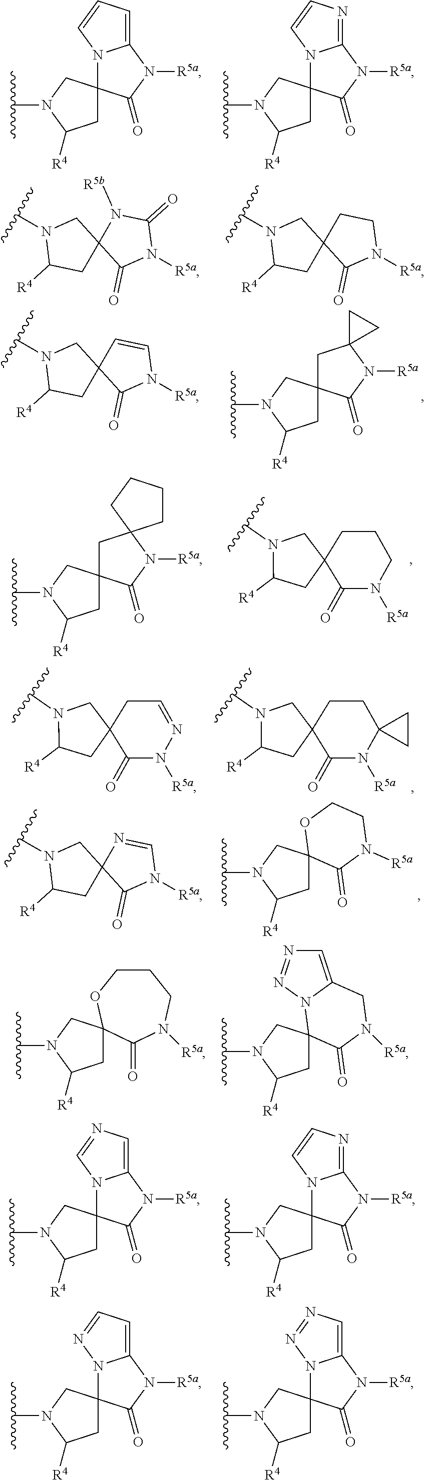 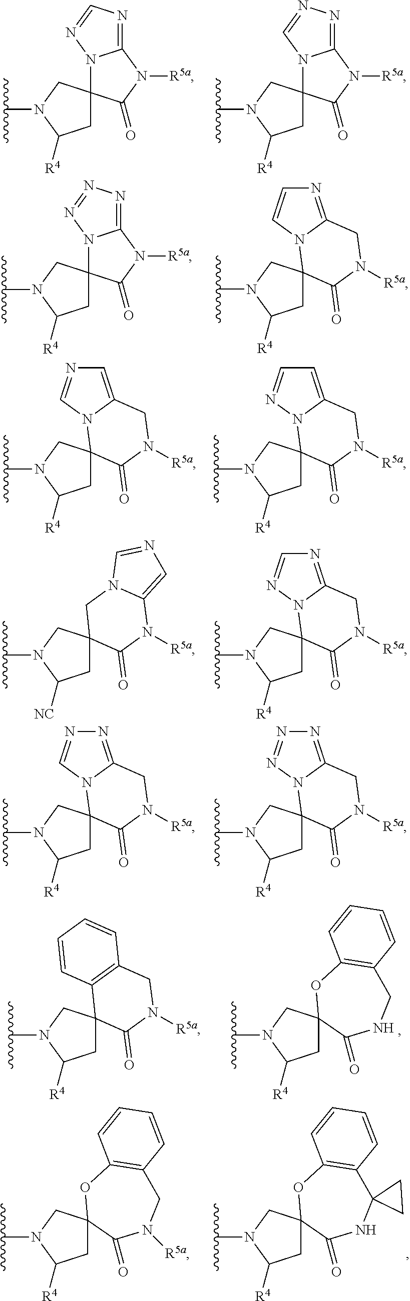 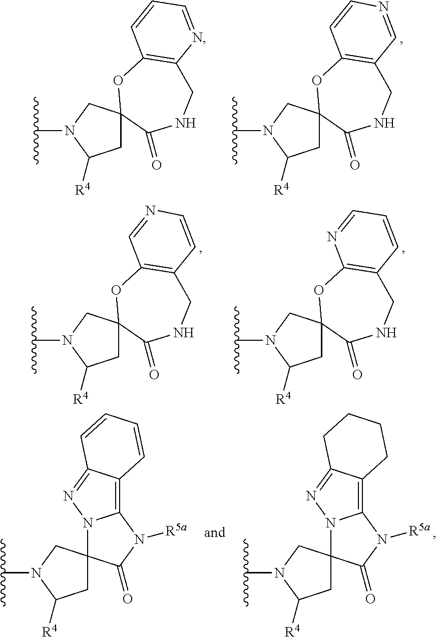
wherein each is unsubstituted or substituted.

26. The compound of claim 1, wherein R¹ is selected from the group consisting of:

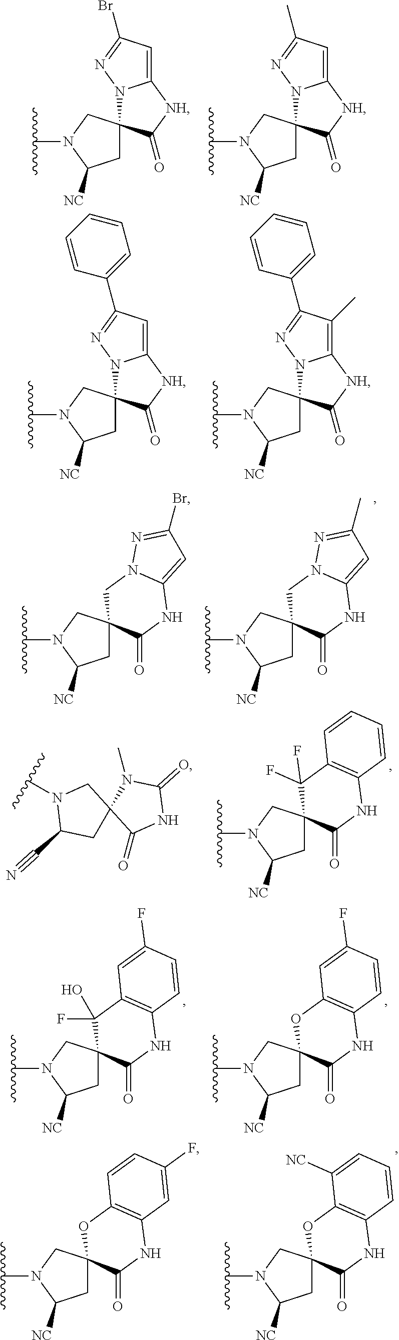

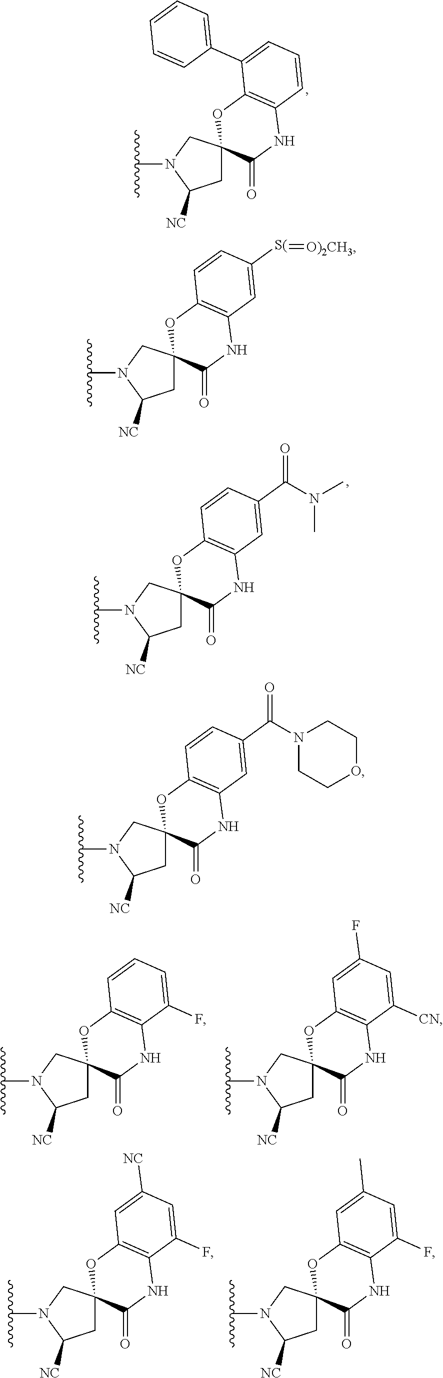

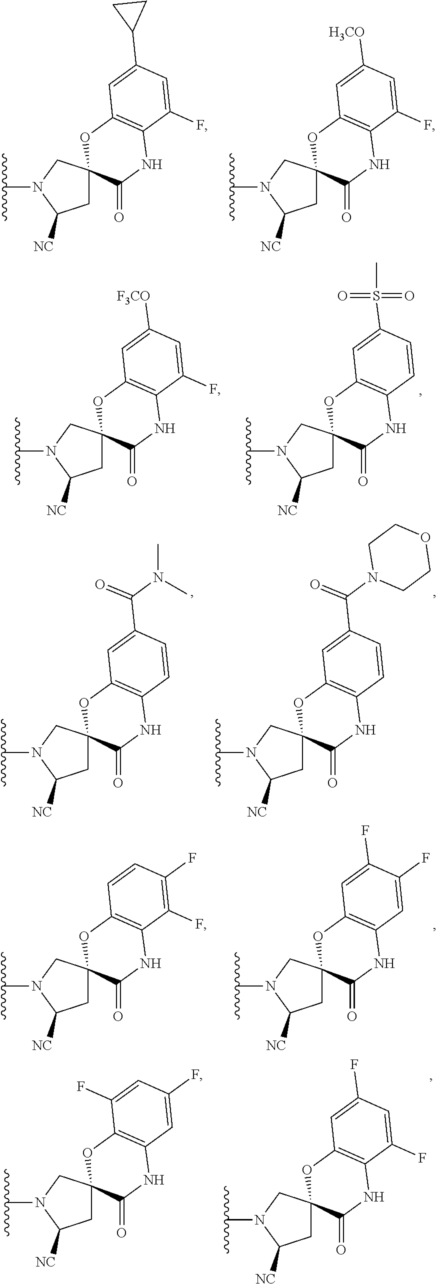

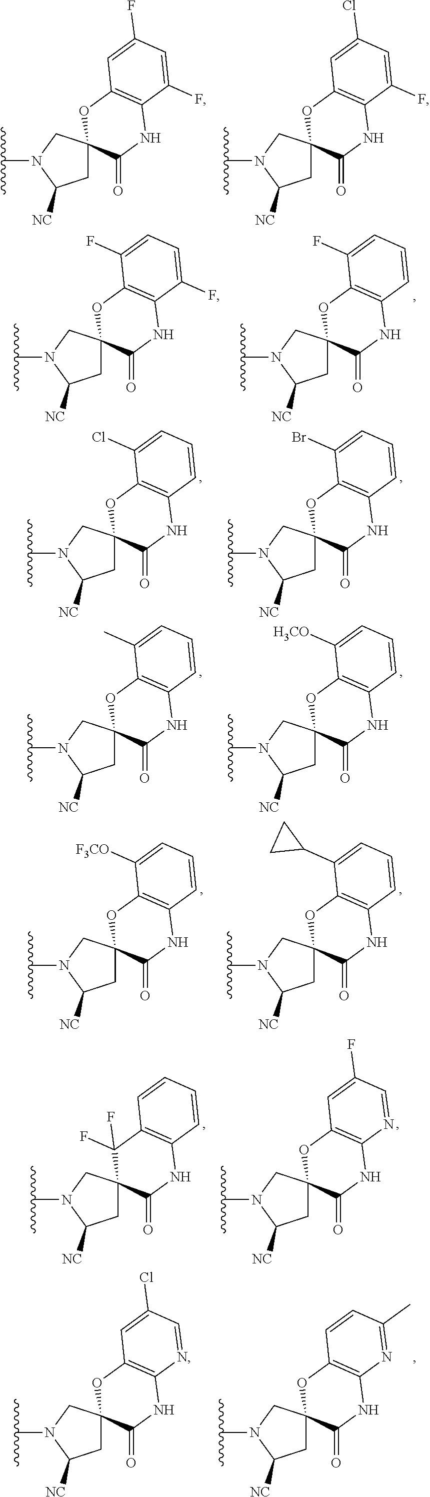

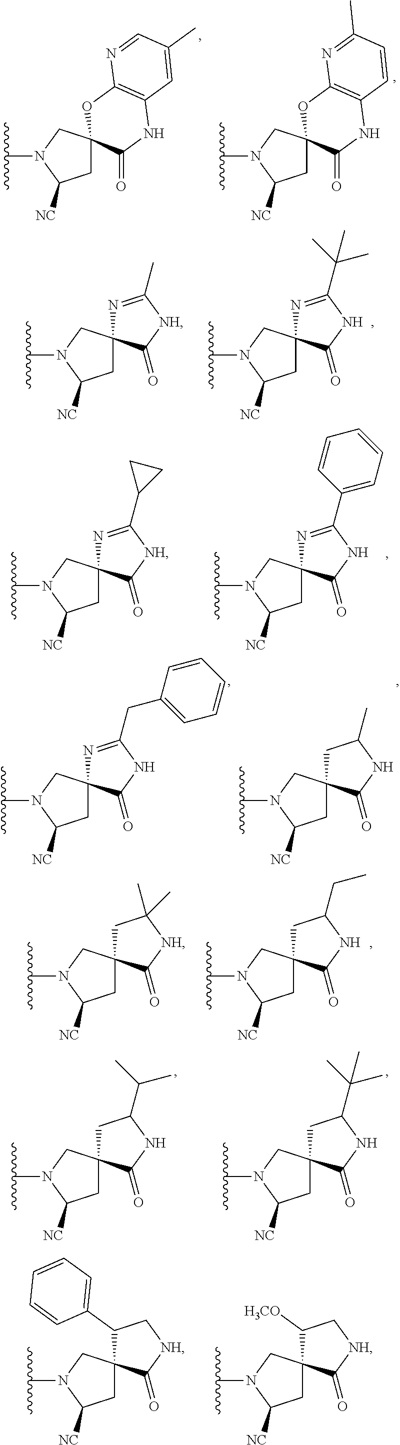

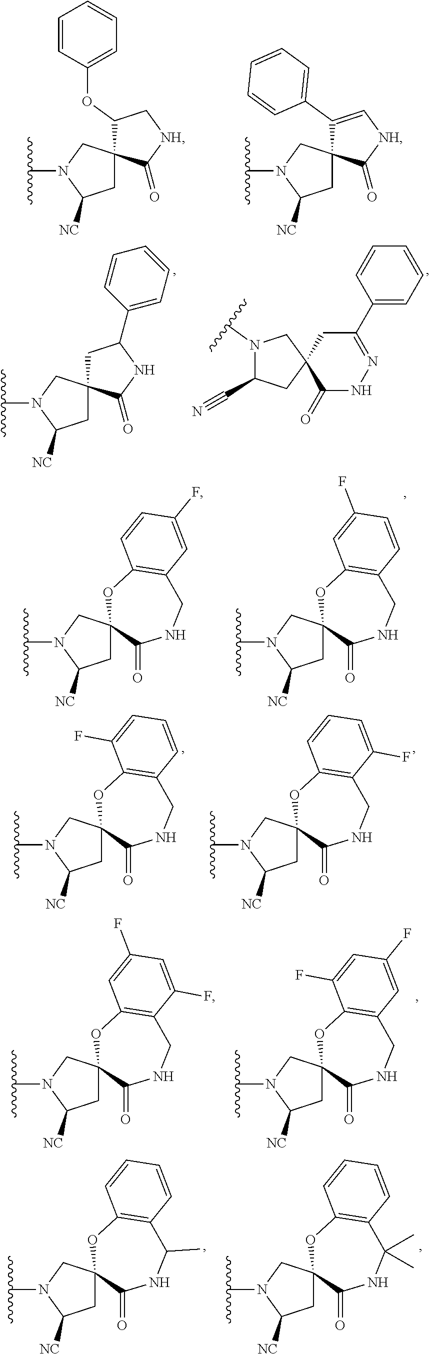

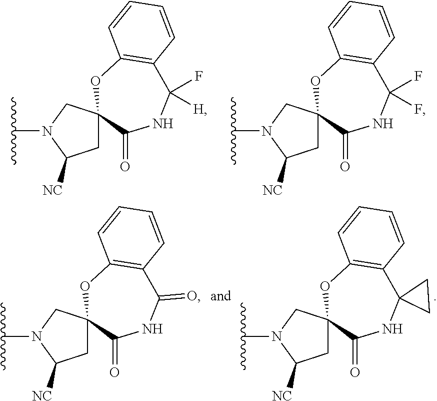

27. (canceled)

28. The compound of claim 1, wherein R² is an unsubstituted C₁₋₈ alkyl.

29. (canceled)

30. (canceled)

31. (canceled)

32. The compound of claim 1, wherein R² is —(CH₂)-(an unsubstituted or a substituted monocyclic C₃₋₆ cycloalkyl).

33. (canceled)

34. (canceled)

35. (canceled)

36. The compound of claim 1, wherein R² is selected from the group consisting of:

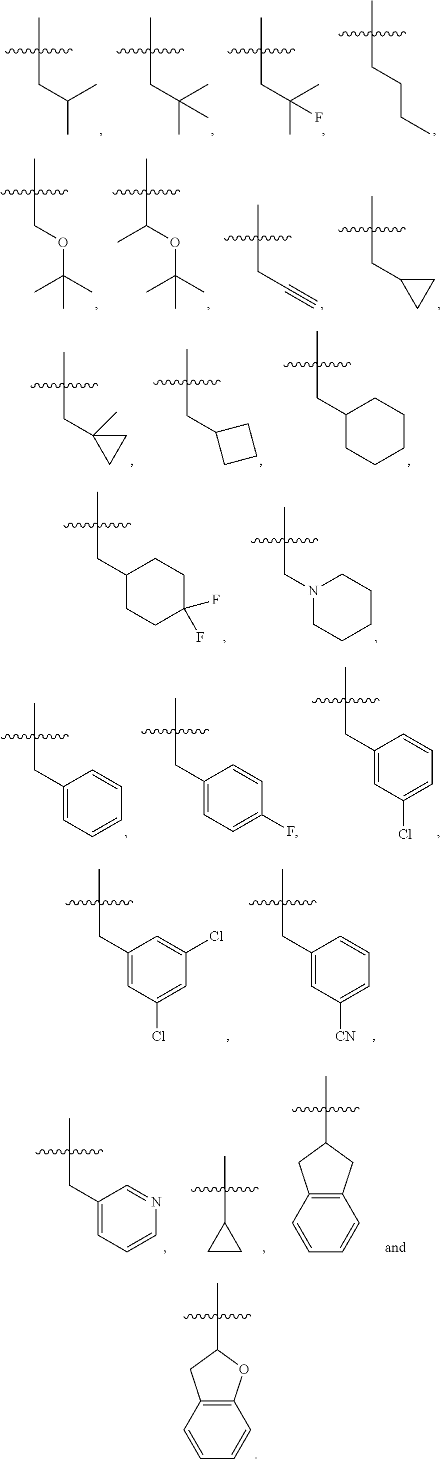

37. The compound of claim 36, wherein R² is

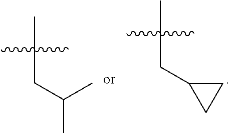

38.-43. (canceled)

44. The compound of claim 1, wherein R⁴ is cyano.

45. (canceled)

46. The compound of am claim 1, wherein R³ is 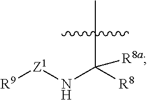
R^{8a} is hydrogen; and Z¹ is —C(═O)—.

47.-73. (canceled)

74. The compound of claim 1, wherein R³ is 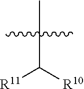
or R¹².

75.-103. (canceled)

104. The compound of claim 74, wherein R¹² is selected from the group consisting of: 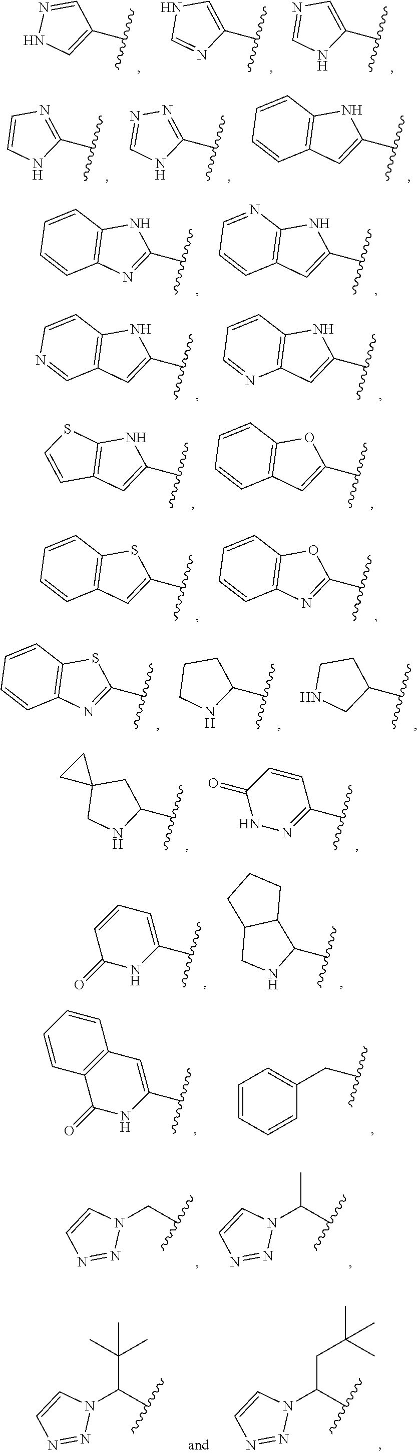
wherein each is unsubstituted or substituted.

105.-114. (canceled)

115. The compound of claim 46, wherein R³ is selected from the group consisting of:

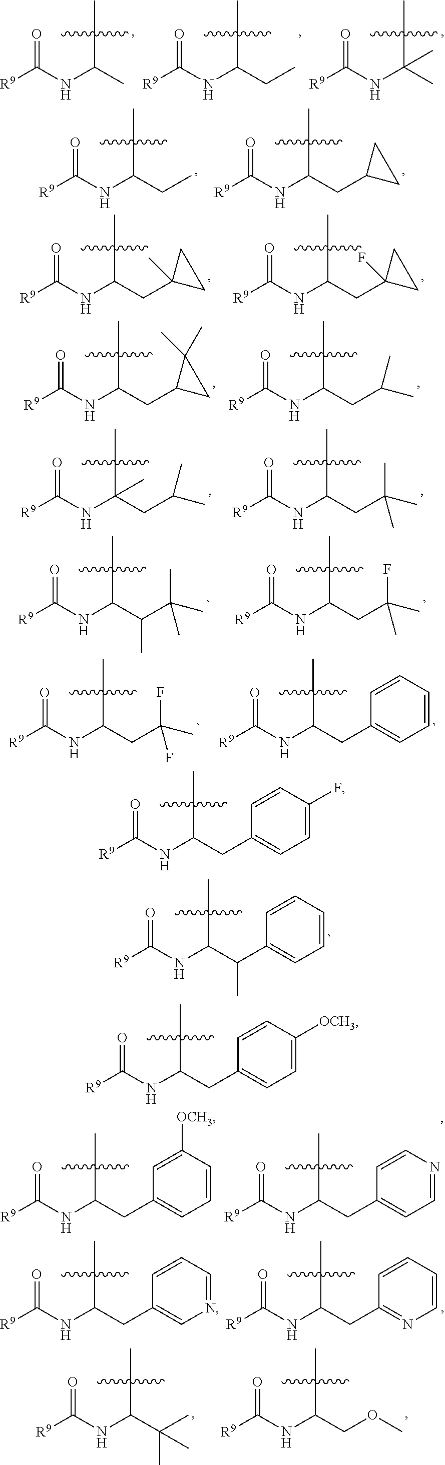

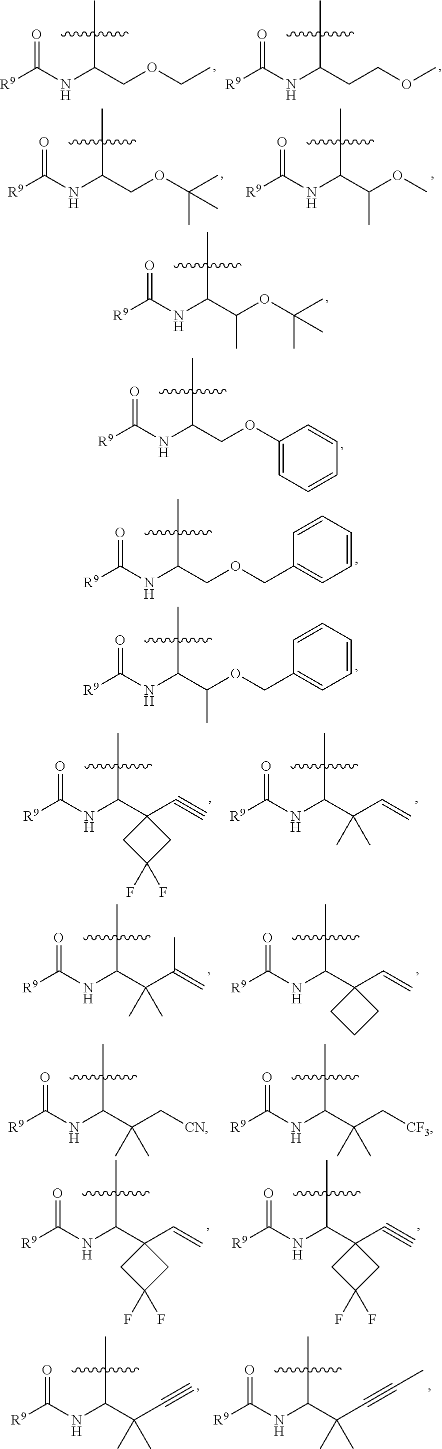

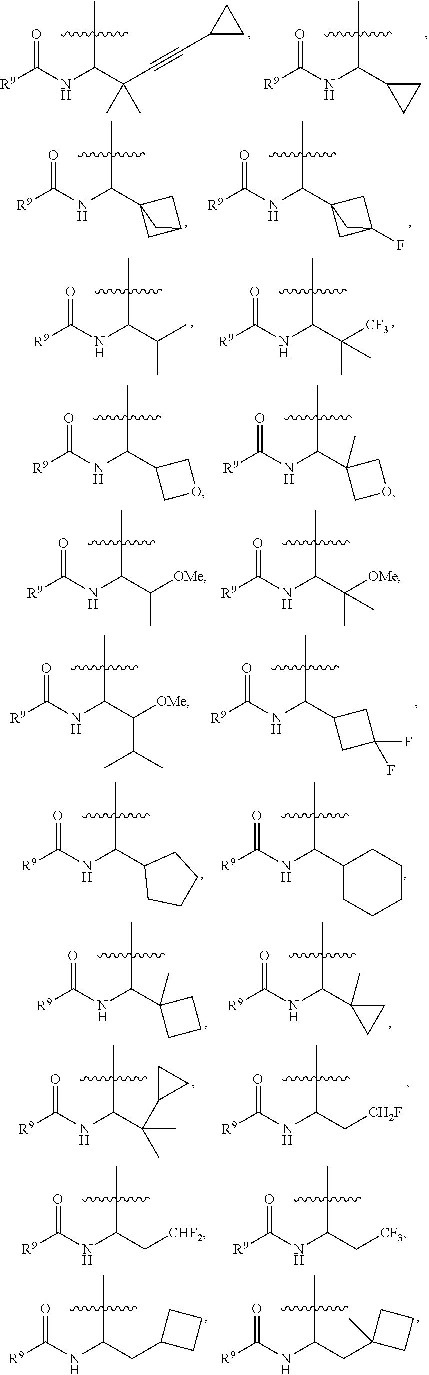

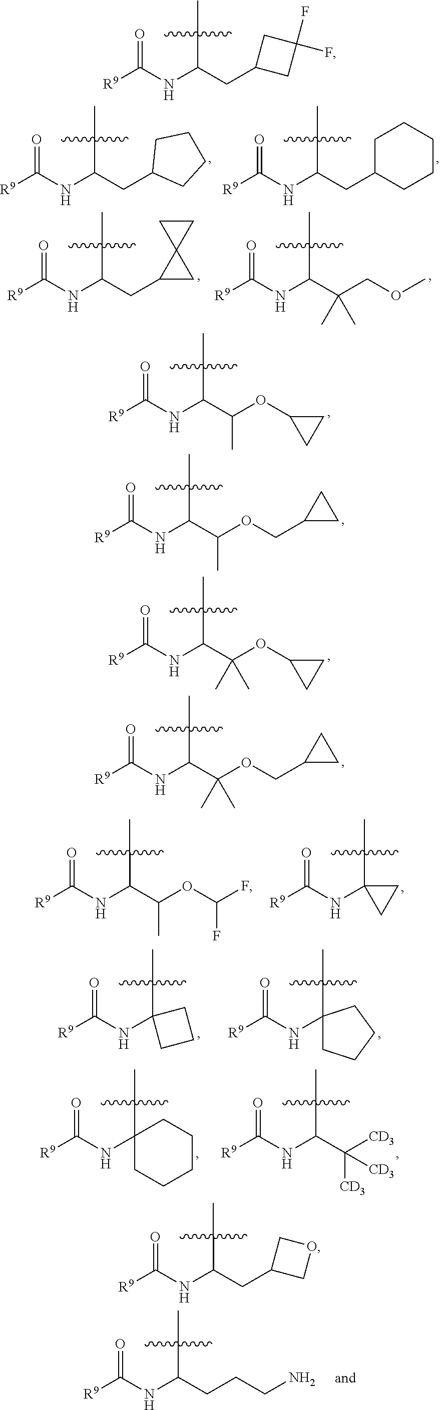

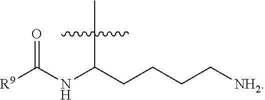

116. The compound of claim 46, wherein R³ is selected from the group consisting of: 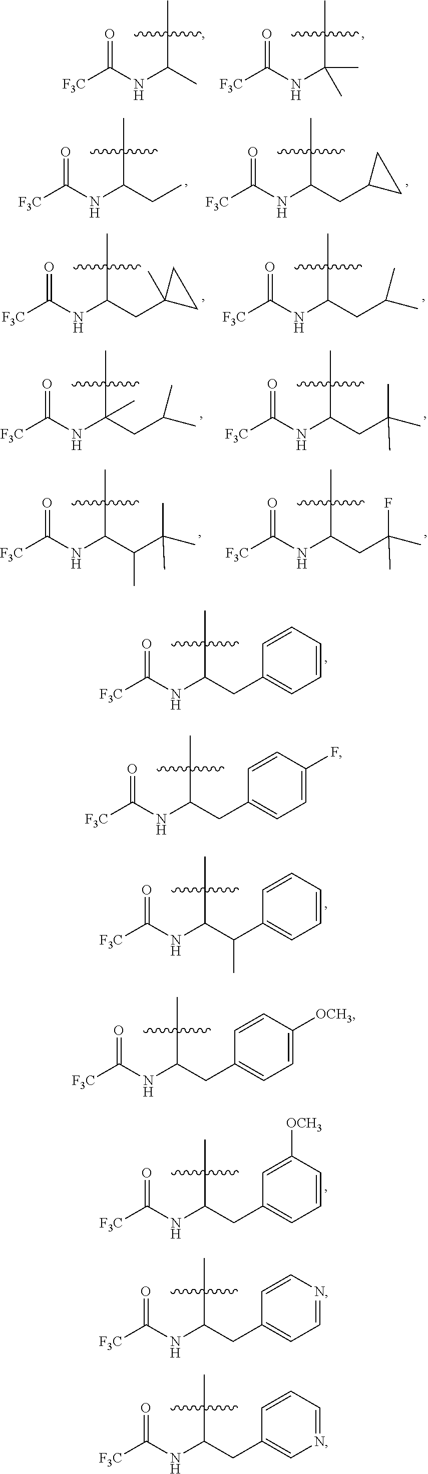 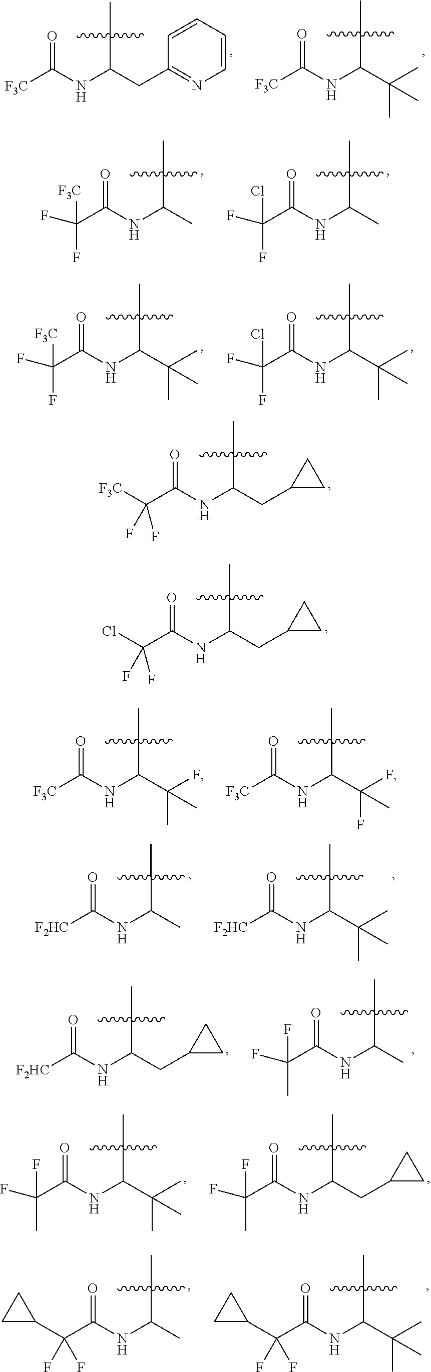 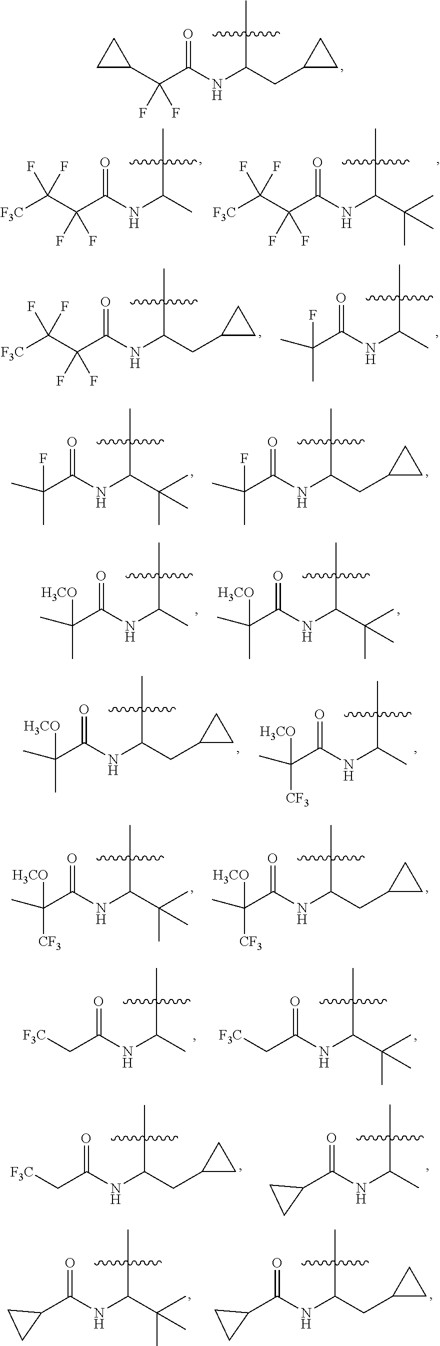 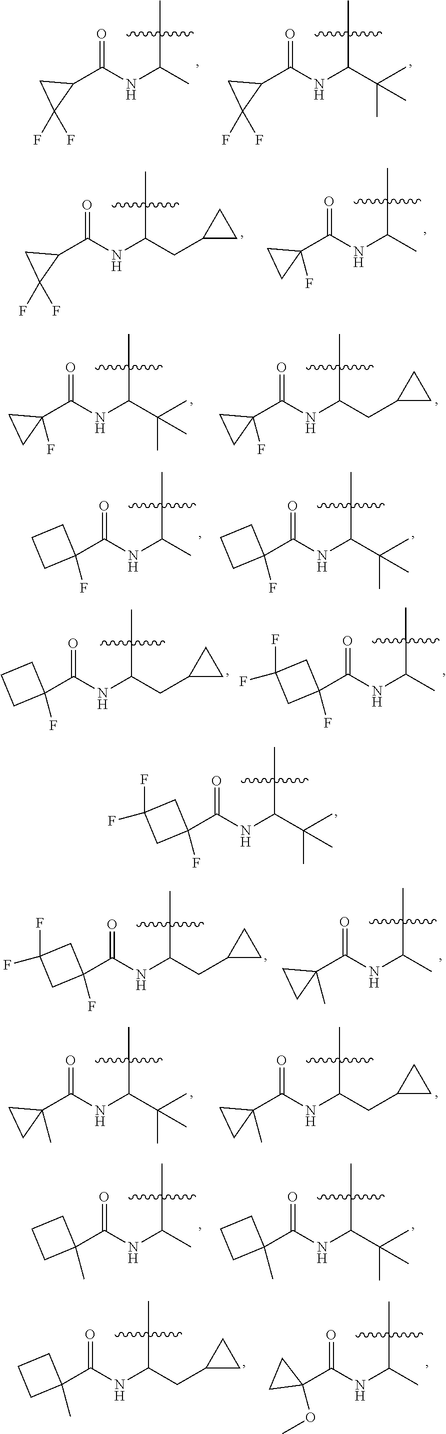 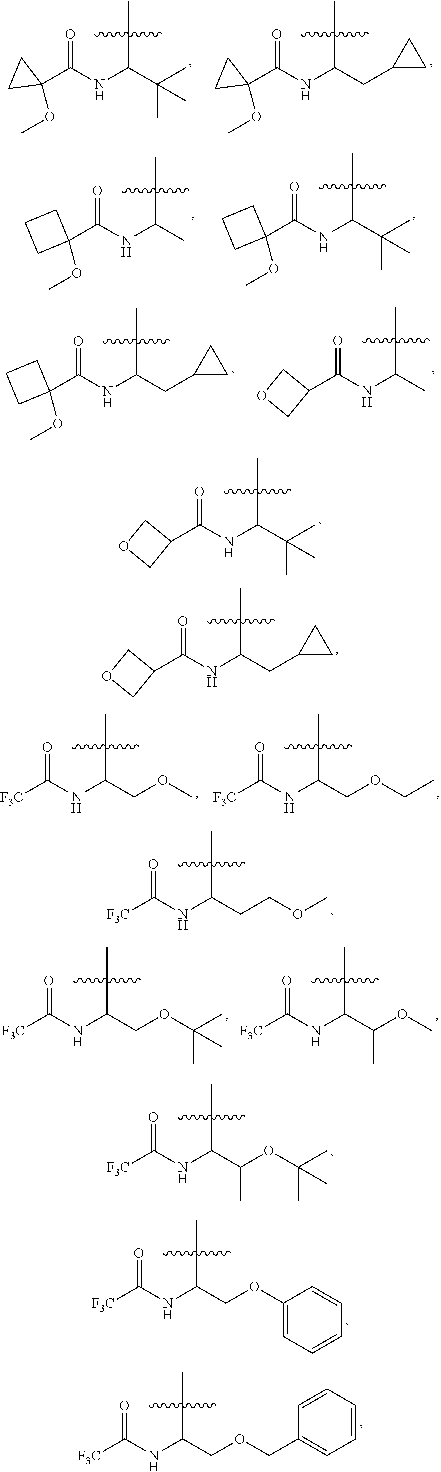 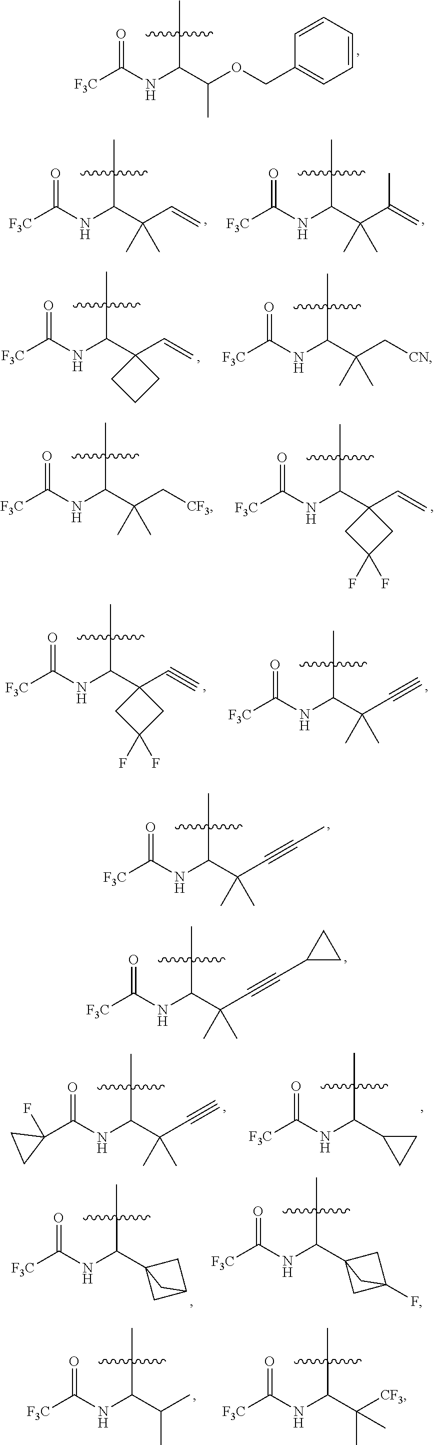 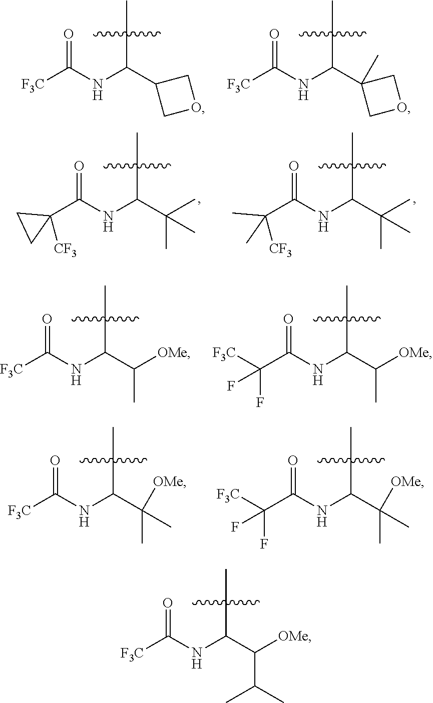 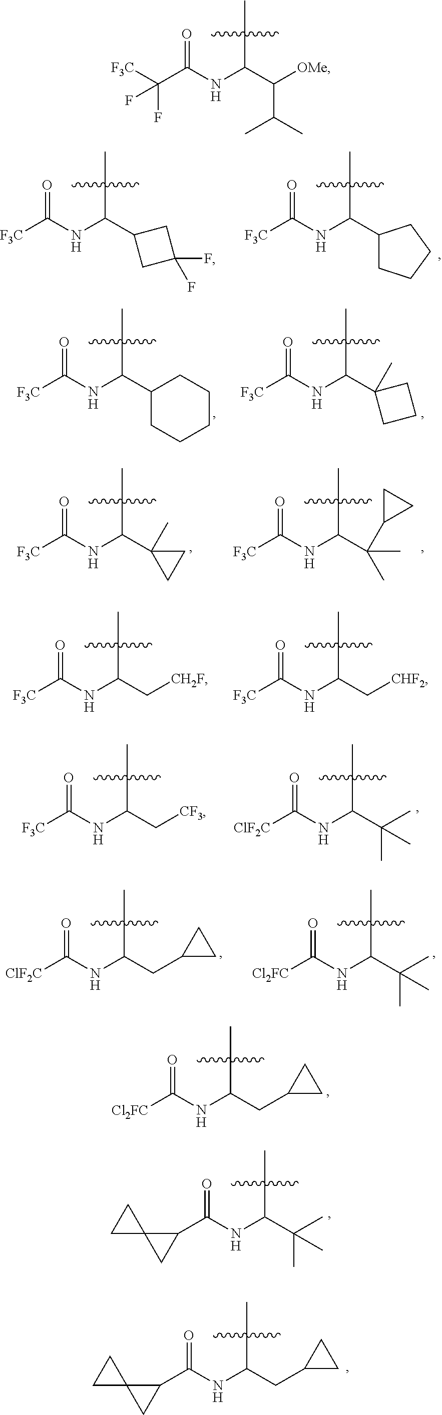 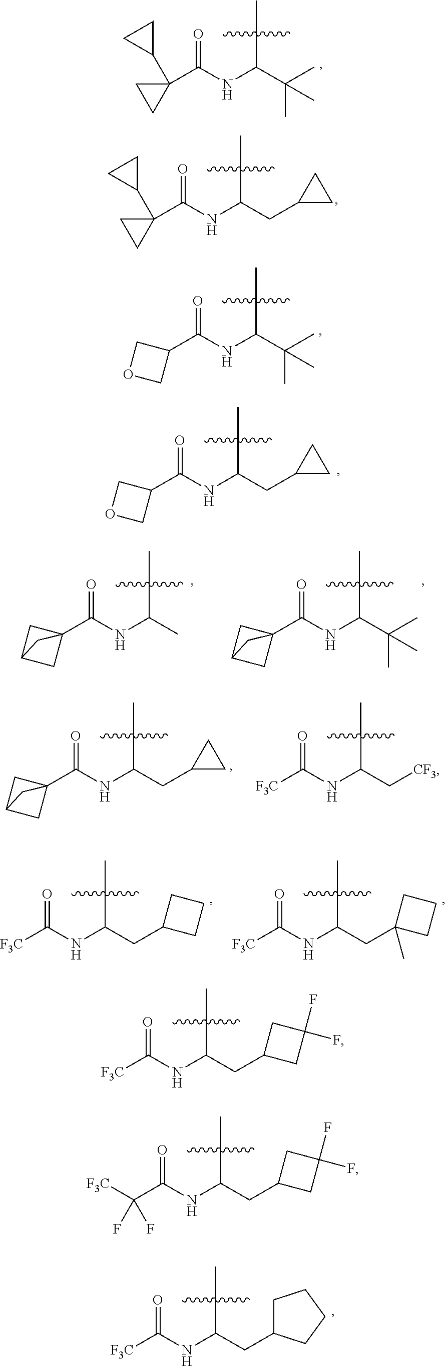 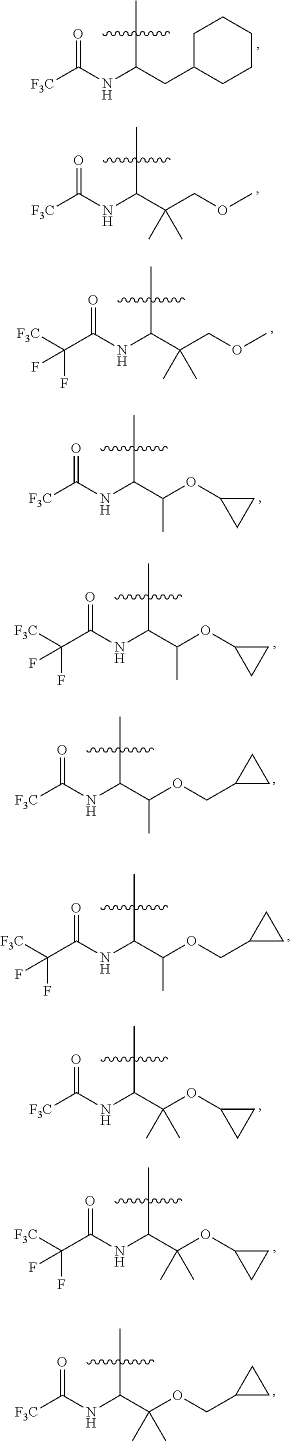 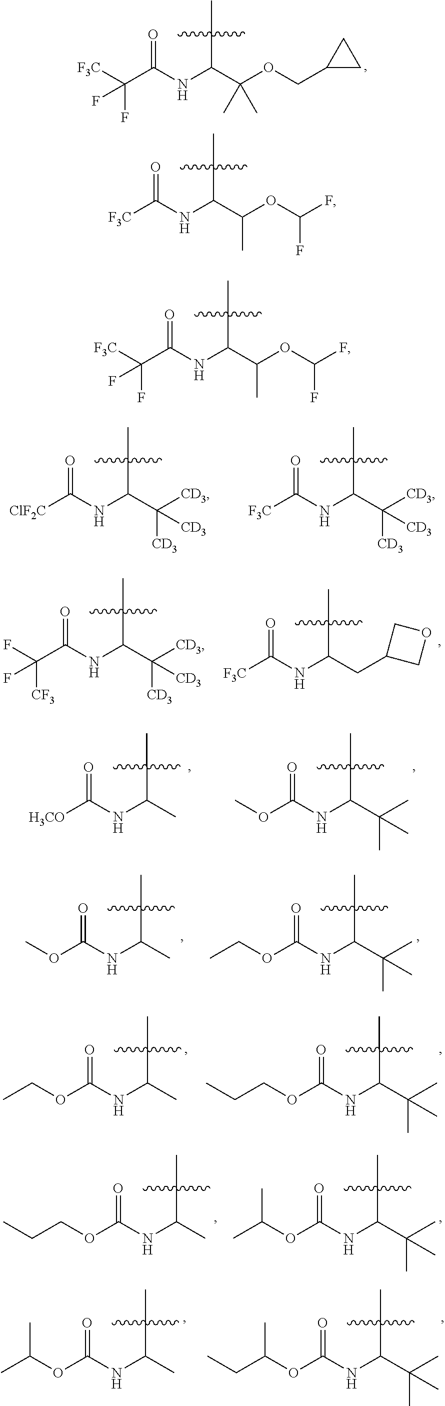 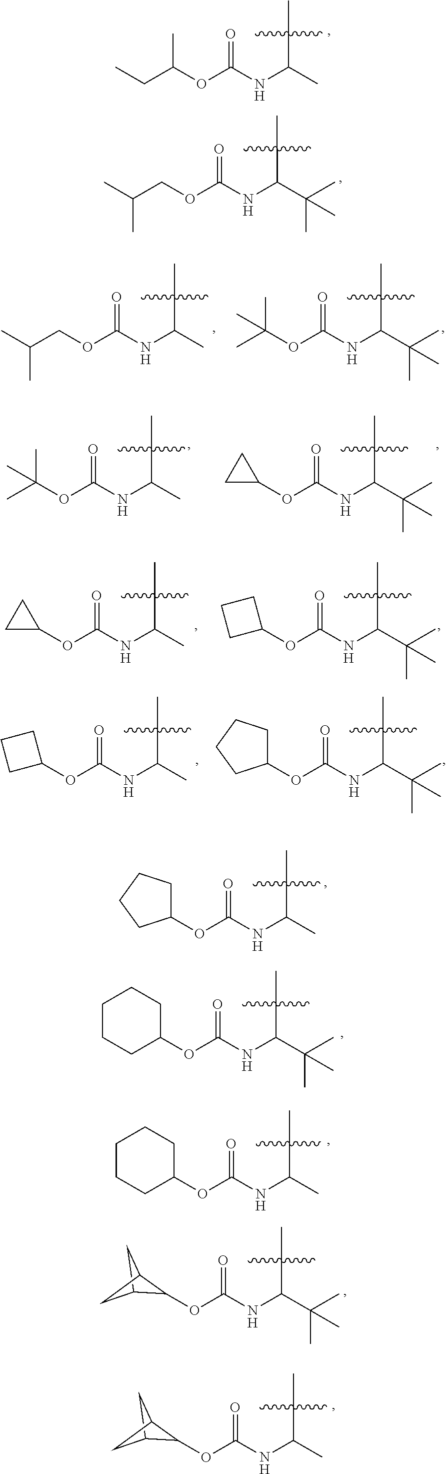 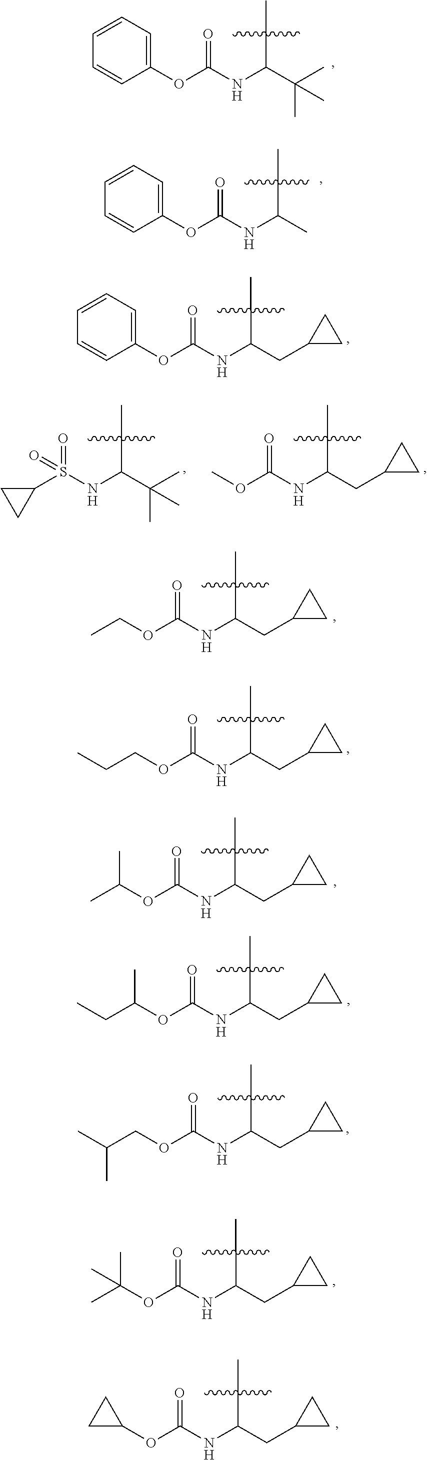 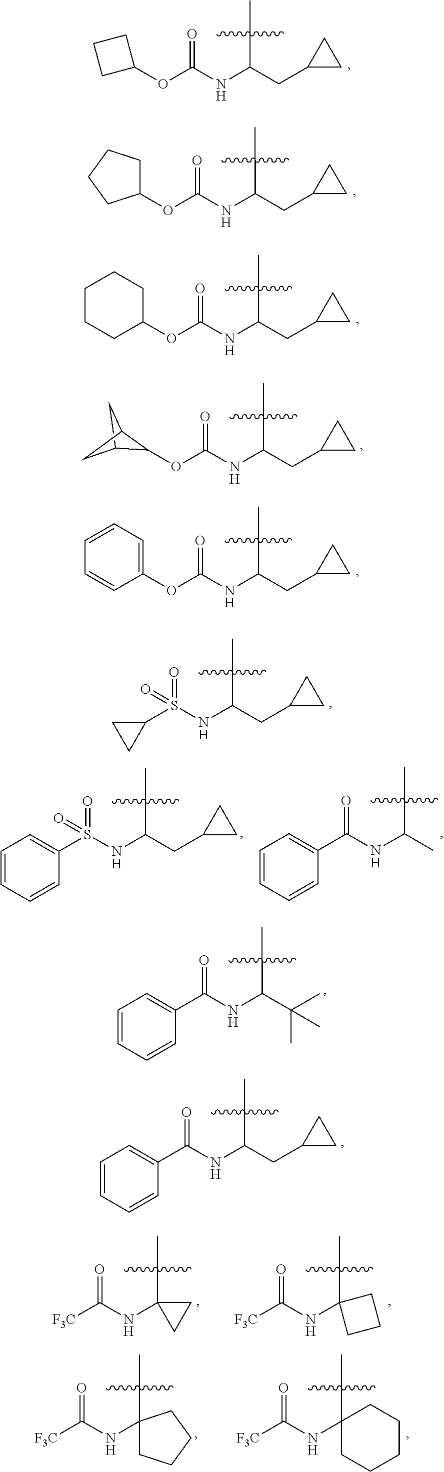 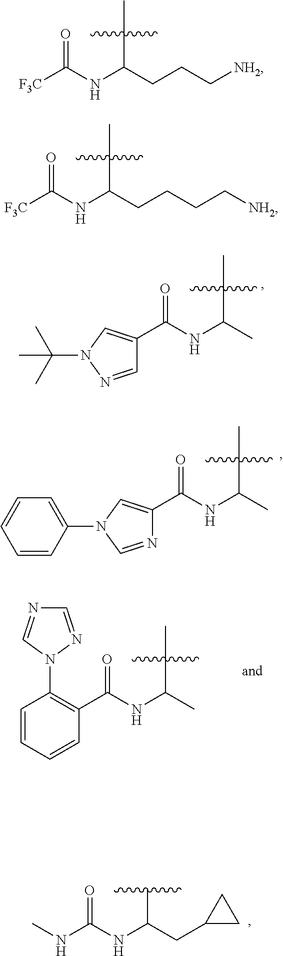
wherein each phenyl and can be substituted or unsubstituted.

117. The compound of claim 74, wherein R³ is selected from the group consisting of: 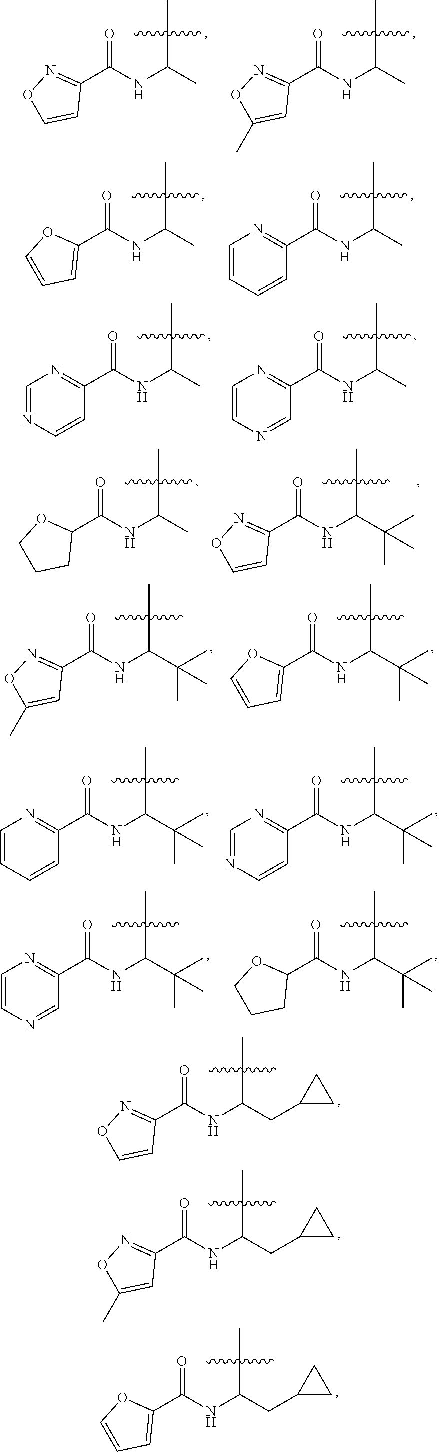 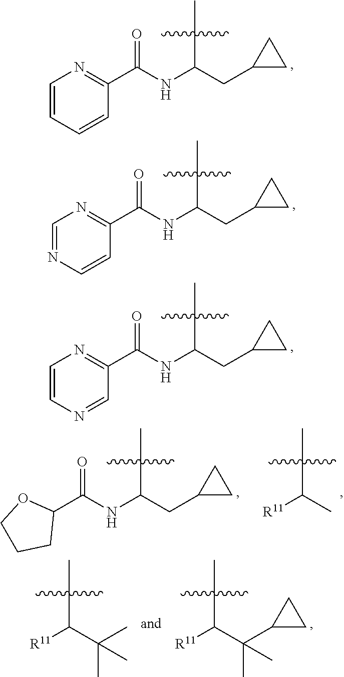
wherein each moiety is unsubstituted or substituted.

118.-122. (canceled)

123. The compound of claim 1, wherein the compound is selected from the group consisting of: 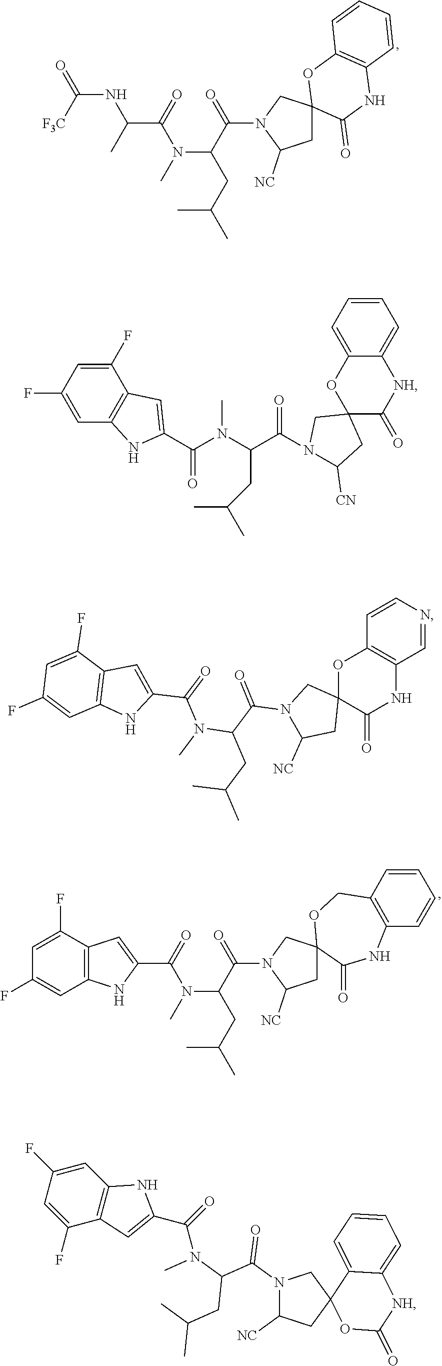 [SMILES: CC(C)CC(C(=O)N1CC2(CC1C#N)Oc1ccccc1NC2=O)N(C)C(=O)C(C)NC(=O)C(F)(F)F ; CC(C)CC(C(=O)N1CC2(CC1C#N)Oc1ccccc1NC2=O)N(C)C(=O)c1cc2c(F)cc(F)cc2[nH]1 ; CC(C)CC(C(=O)N1CC2(CC1C#N)Oc1ccncc1NC2=O)N(C)C(=O)c1cc2c(F)cc(F)cc2[nH]1 ; CC(C)CC(C(=O)N1CC2(CC1C#N)OCc1ccccc1NC2=O)N(C)C(=O)c1cc2c(F)cc(F)cc2[nH]1 ; CC(C)CC(C(=O)N1CC2(CC1C#N)OC(=O)Nc1ccccc12)N(C)C(=O)c1cc2c(F)cc(F)cc2[nH]1] 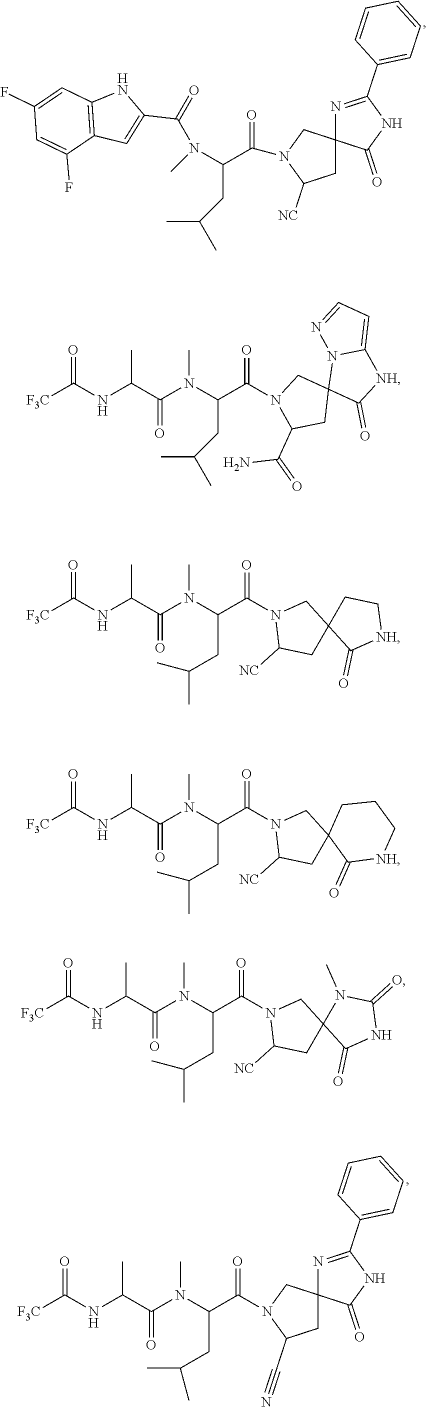 [SMILES: CC(C)CC(C(=O)N1CC2(CC1C#N)N=C(c1ccccc1)NC2=O)N(C)C(=O)c1cc2c(F)cc(F)cc2[nH]1 ; CC(C)CC(C(=O)N1CC2(CC1C(N)=O)C(=O)Nc1ccnn12)N(C)C(=O)C(C)NC(=O)C(F)(F)F ; CC(C)CC(C(=O)N1CC2(CCNC2=O)CC1C#N)N(C)C(=O)C(C)NC(=O)C(F)(F)F ; CC(C)CC(C(=O)N1CC2(CCCNC2=O)CC1C#N)N(C)C(=O)C(C)NC(=O)C(F)(F)F ; CC(C)CC(C(=O)N1CC2(CC1C#N)C(=O)NC(=O)N2C)N(C)C(=O)C(C)NC(=O)C(F)(F)F ; CC(C)CC(C(=O)N1CC2(CC1C#N)N=C(c1ccccc1)NC2=O)N(C)C(=O)C(C)NC(=O)C(F)(F)F] 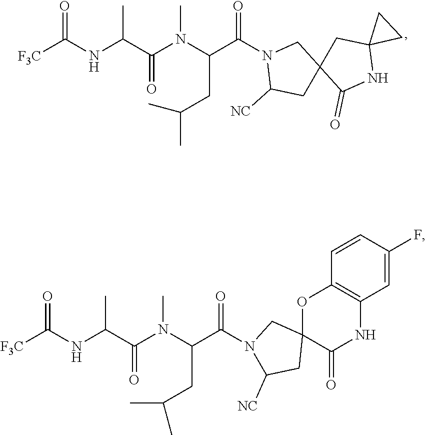 [SMILES: CC(C)CC(C(=O)N1CC2(CC1C#N)CC1(CC1)NC2=O)N(C)C(=O)C(C)NC(=O)C(F)(F)F ; CC(C)CC(C(=O)N1CC2(CC1C#N)Oc1ccc(F)cc1NC2=O)N(C)C(=O)C(C)NC(=O)C(F)(F)F] 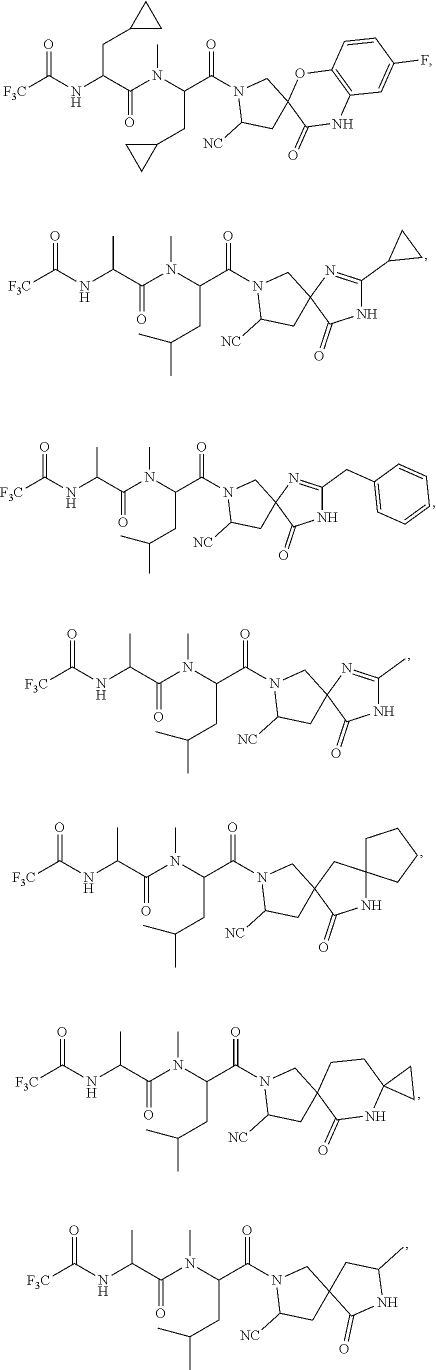 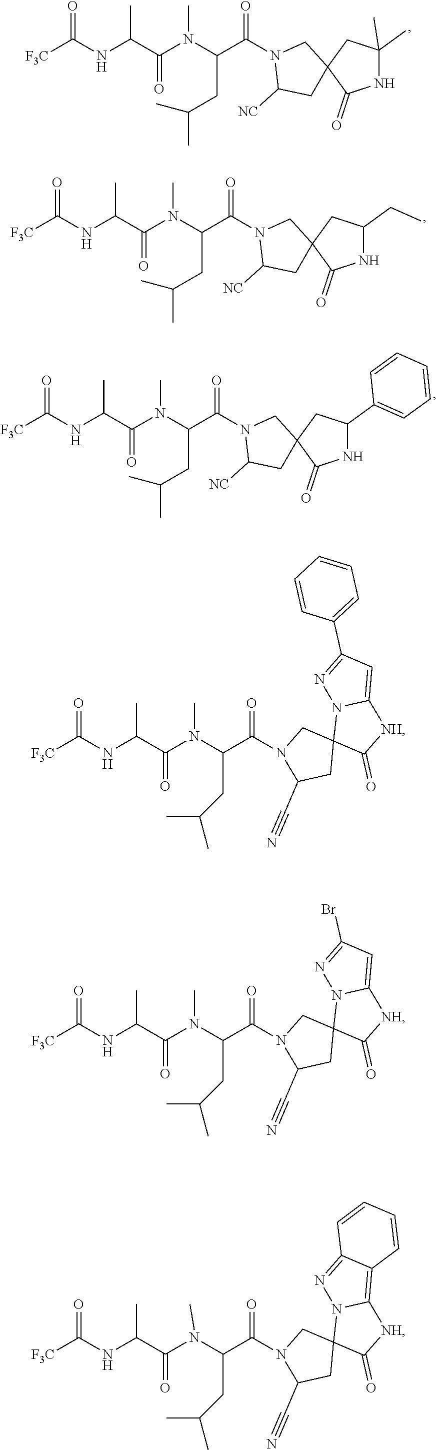 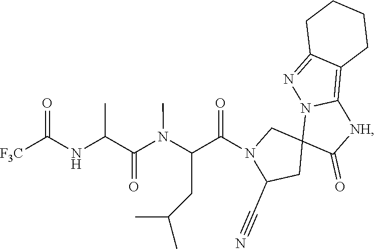 [SMILES: CC(C)CC(C(=O)N1CC2(CC1C#N)C(=O)Nc1c3c(nn12)CCCC3)N(C)C(=O)C(C)NC(=O)C(F)(F)F] 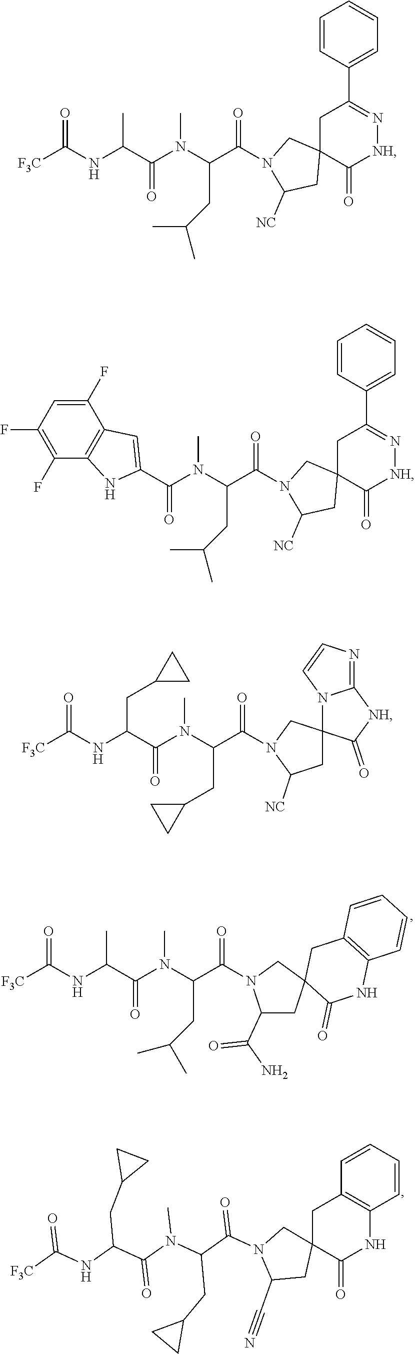 [SMILES: CC(C)CC(C(=O)N1CC2(CC(c3ccccc3)=NNC2=O)CC1C#N)N(C)C(=O)C(C)NC(=O)C(F)(F)F ; CC(C)CC(C(=O)N1CC2(CC(c3ccccc3)=NNC2=O)CC1C#N)N(C)C(=O)c1cc2c(F)cc(F)c(F)c2[nH]1 ; CN(C(=O)C(CC1CC1)NC(=O)C(F)(F)F)C(CC1CC1)C(=O)N1CC2(CC1C#N)C(=O)Nc1nccn12 ; CC(C)CC(C(=O)N1CC2(Cc3ccccc3NC2=O)CC1C(N)=O)N(C)C(=O)C(C)NC(=O)C(F)(F)F ; CN(C(=O)C(CC1CC1)NC(=O)C(F)(F)F)C(CC1CC1)C(=O)N1CC2(Cc3ccccc3NC2=O)CC1C#N] 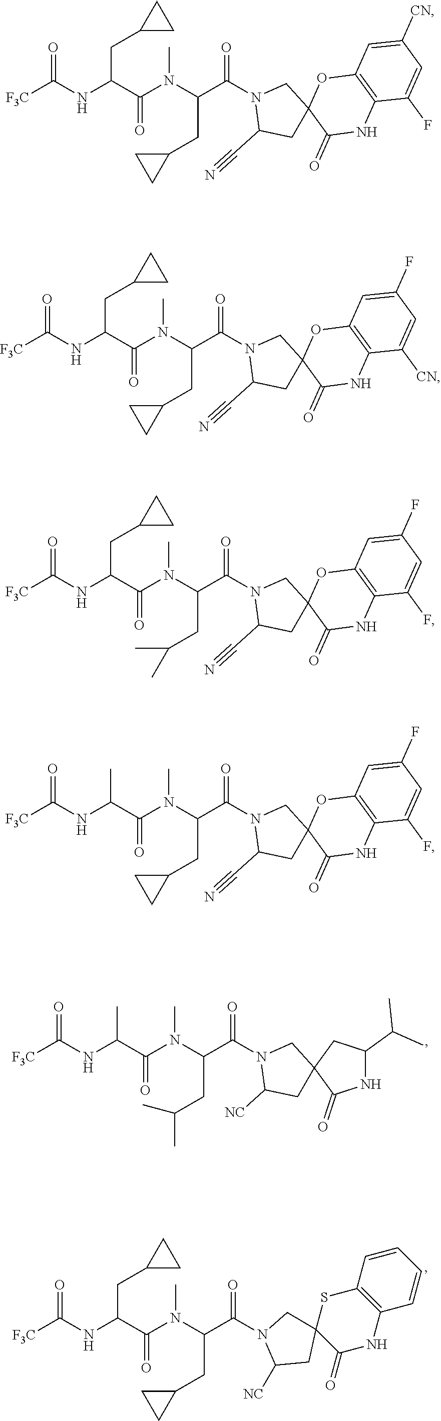 [SMILES: CN(C(=O)C(CC1CC1)NC(=O)C(F)(F)F)C(CC1CC1)C(=O)N1CC2(CC1C#N)Oc1cc(C#N)cc(F)c1NC2=O ; CN(C(=O)C(CC1CC1)NC(=O)C(F)(F)F)C(CC1CC1)C(=O)N1CC2(CC1C#N)Oc1cc(F)cc(C#N)c1NC2=O ; CC(C)CC(C(=O)N1CC2(CC1C#N)Oc1cc(F)cc(F)c1NC2=O)N(C)C(=O)C(CC1CC1)NC(=O)C(F)(F)F ; CC(NC(=O)C(F)(F)F)C(=O)N(C)C(CC1CC1)C(=O)N1CC2(CC1C#N)Oc1cc(F)cc(F)c1NC2=O ; CC(C)CC(C(=O)N1CC2(CC(C(C)C)NC2=O)CC1C#N)N(C)C(=O)C(C)NC(=O)C(F)(F)F ; CN(C(=O)C(CC1CC1)NC(=O)C(F)(F)F)C(CC1CC1)C(=O)N1CC2(CC1C#N)Sc1ccccc1NC2=O] 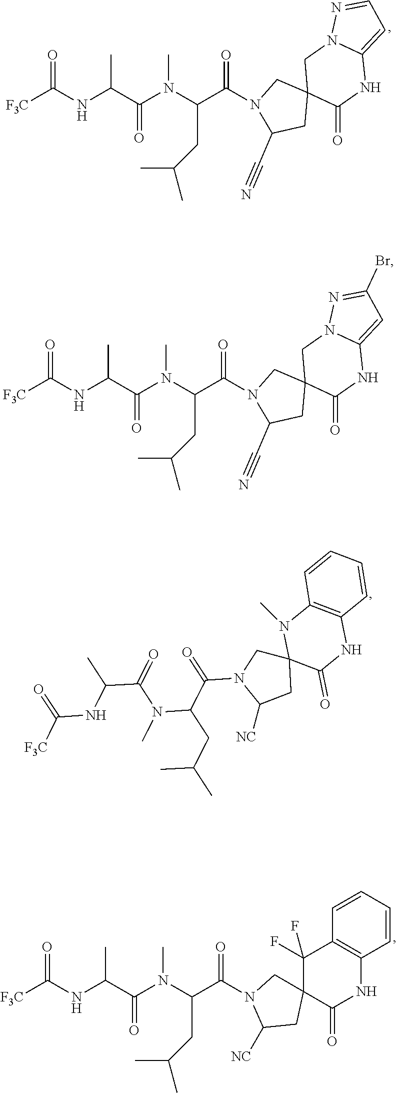 [SMILES: CC(C)CC(C(=O)N1CC2(CC1C#N)Cn1nccc1NC2=O)N(C)C(=O)C(C)NC(=O)C(F)(F)F ; CC(C)CC(C(=O)N1CC2(CC1C#N)Cn1nc(Br)cc1NC2=O)N(C)C(=O)C(C)NC(=O)C(F)(F)F ; CC(C)CC(C(=O)N1CC2(CC1C#N)C(=O)Nc1ccccc1N2C)N(C)C(=O)C(C)NC(=O)C(F)(F)F ; CC(C)CC(C(=O)N1CC2(CC1C#N)C(=O)Nc1ccccc1C2(F)F)N(C)C(=O)C(C)NC(=O)C(F)(F)F] 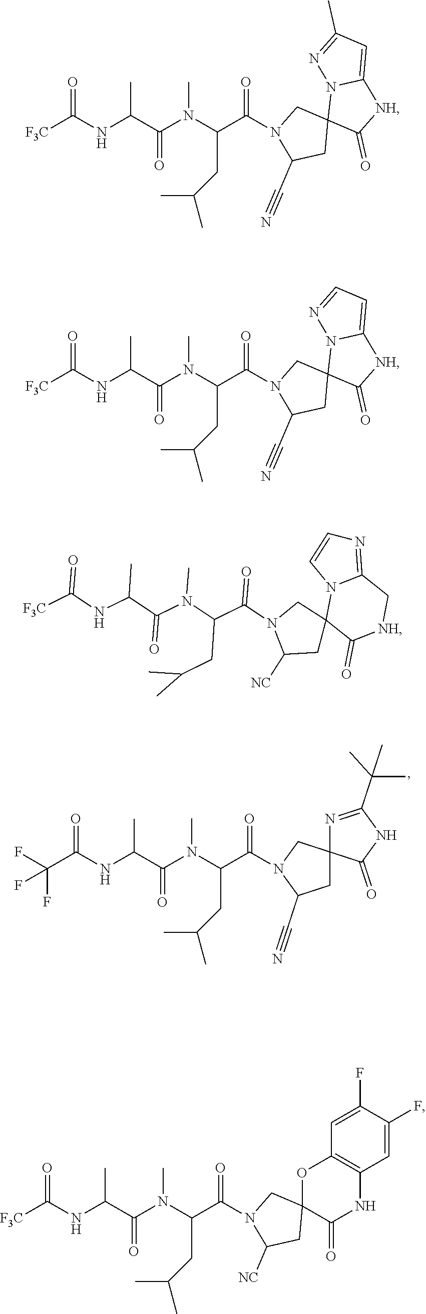 [SMILES: Cc1cc2n(n1)C1(CC(C#N)N(C(=O)C(CC(C)C)N(C)C(=O)C(C)NC(=O)C(F)(F)F)C1)C(=O)N2 ; CC(C)CC(C(=O)N1CC2(CC1C#N)C(=O)Nc1ccnn12)N(C)C(=O)C(C)NC(=O)C(F)(F)F ; CC(C)CC(C(=O)N1CC2(CC1C#N)C(=O)NCc1nccn12)N(C)C(=O)C(C)NC(=O)C(F)(F)F ; CC(C)CC(C(=O)N1CC2(CC1C#N)N=C(C(C)(C)C)NC2=O)N(C)C(=O)C(C)NC(=O)C(F)(F)F ; CC(C)CC(C(=O)N1CC2(CC1C#N)Oc1cc(F)c(F)cc1NC2=O)N(C)C(=O)C(C)NC(=O)C(F)(F)F] 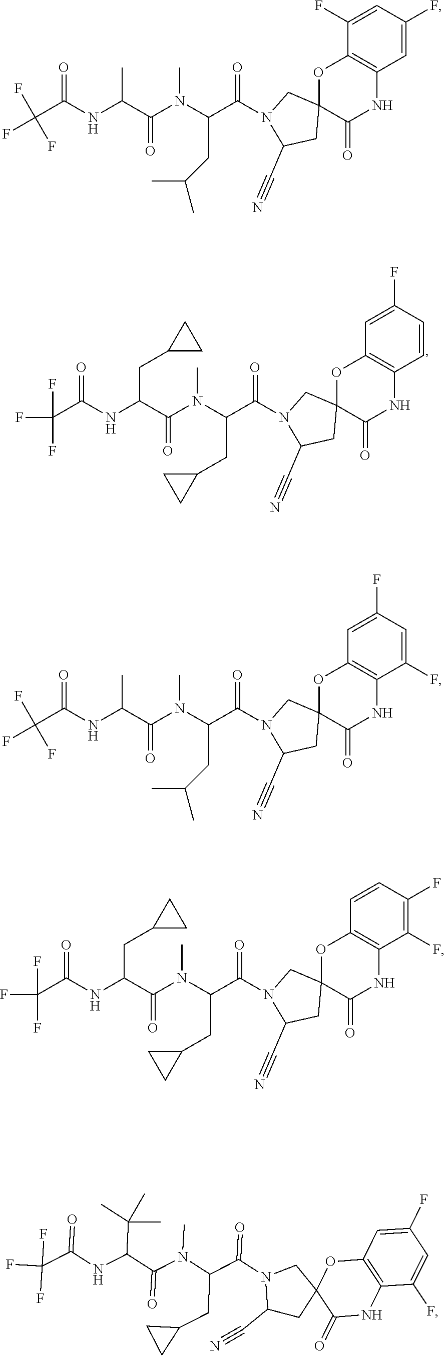 [SMILES: CC(C)CC(C(=O)N1CC2(CC1C#N)Oc1c(F)cc(F)cc1NC2=O)N(C)C(=O)C(C)NC(=O)C(F)(F)F ; CN(C(=O)C(CC1CC1)NC(=O)C(F)(F)F)C(CC1CC1)C(=O)N1CC2(CC1C#N)Oc1cc(F)ccc1NC2=O ; CC(C)CC(C(=O)N1CC2(CC1C#N)Oc1cc(F)cc(F)c1NC2=O)N(C)C(=O)C(C)NC(=O)C(F)(F)F ; CN(C(=O)C(CC1CC1)NC(=O)C(F)(F)F)C(CC1CC1)C(=O)N1CC2(CC1C#N)Oc1ccc(F)c(F)c1NC2=O ; CN(C(=O)C(NC(=O)C(F)(F)F)C(C)(C)C)C(CC1CC1)C(=O)N1CC2(CC1C#N)Oc1cc(F)cc(F)c1NC2=O] 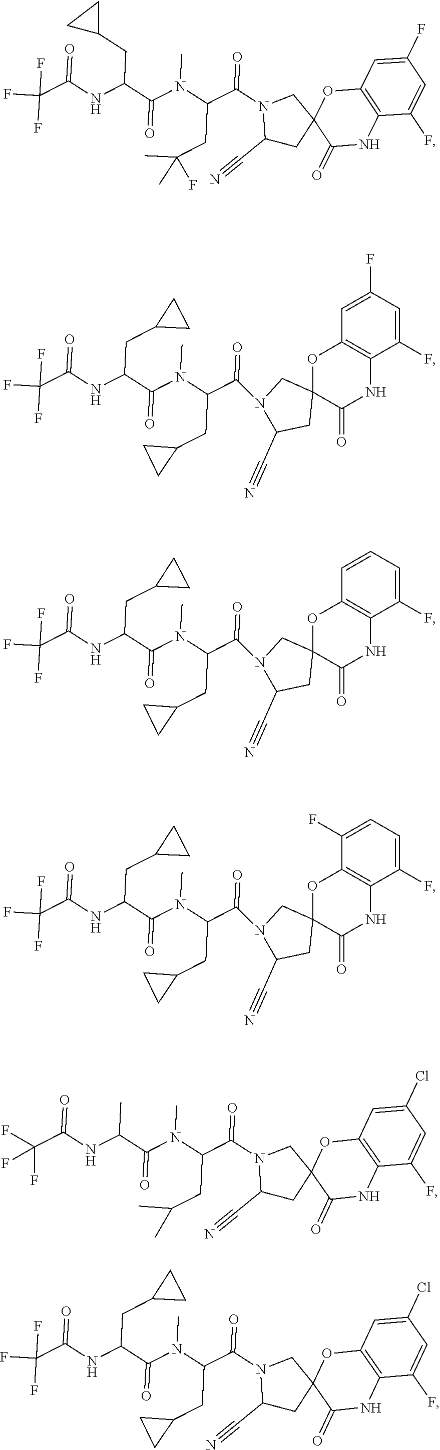 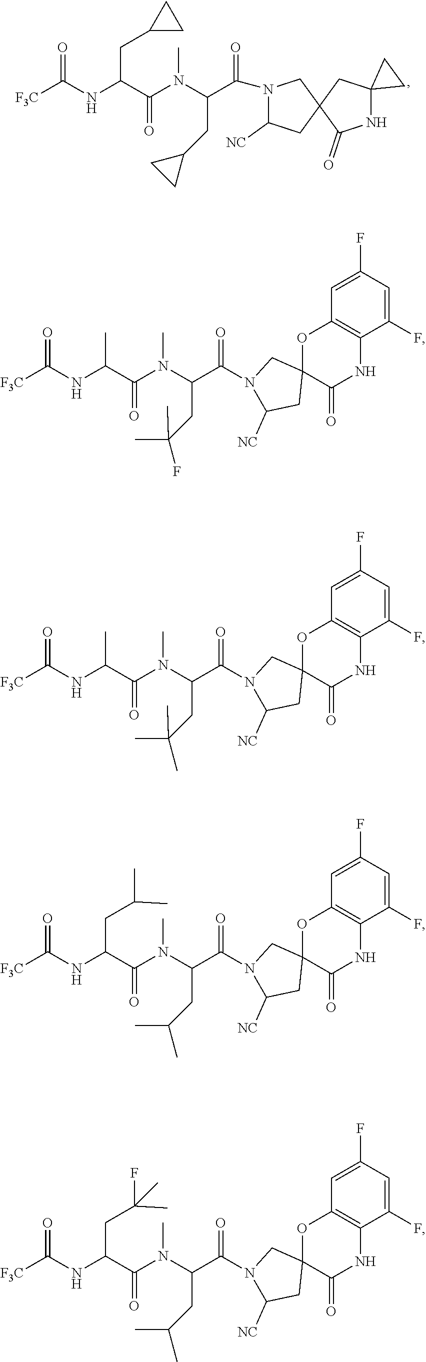 [SMILES: CN(C(=O)C(CC1CC1)NC(=O)C(F)(F)F)C(CC1CC1)C(=O)N1CC2(CC1C#N)CC1(CC1)NC2=O ; CC(NC(=O)C(F)(F)F)C(=O)N(C)C(CC(C)(C)F)C(=O)N1CC2(CC1C#N)Oc1cc(F)cc(F)c1NC2=O ; CC(NC(=O)C(F)(F)F)C(=O)N(C)C(CC(C)(C)C)C(=O)N1CC2(CC1C#N)Oc1cc(F)cc(F)c1NC2=O ; CC(C)CC(NC(=O)C(F)(F)F)C(=O)N(C)C(CC(C)C)C(=O)N1CC2(CC1C#N)Oc1cc(F)cc(F)c1NC2=O ; CC(C)CC(C(=O)N1CC2(CC1C#N)Oc1cc(F)cc(F)c1NC2=O)N(C)C(=O)C(CC(C)(C)F)NC(=O)C(F)(F)F] 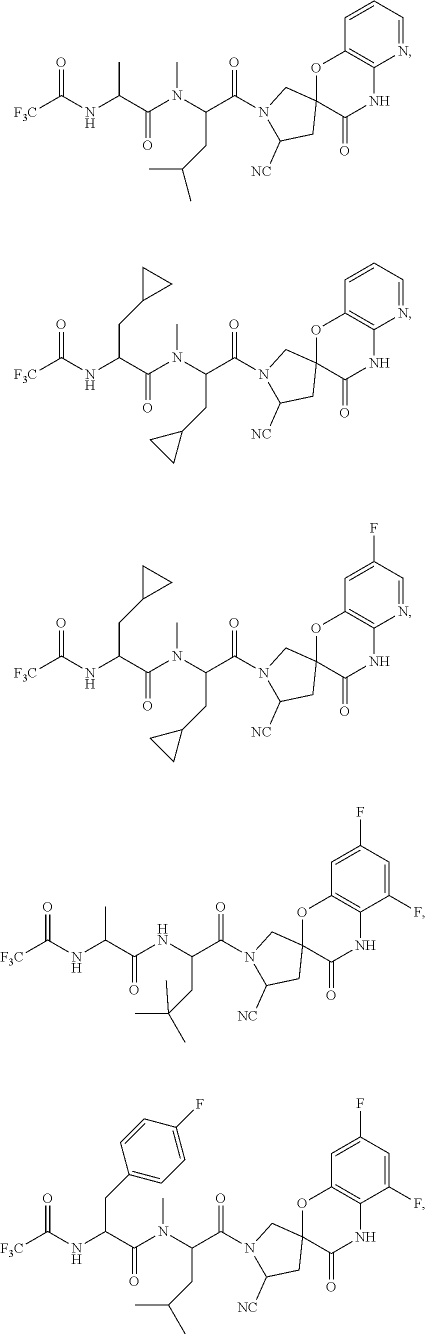 [SMILES: CC(C)CC(C(=O)N1CC2(CC1C#N)Oc1cccnc1NC2=O)N(C)C(=O)C(C)NC(=O)C(F)(F)F ; CN(C(=O)C(CC1CC1)NC(=O)C(F)(F)F)C(CC1CC1)C(=O)N1CC2(CC1C#N)Oc1cccnc1NC2=O ; CN(C(=O)C(CC1CC1)NC(=O)C(F)(F)F)C(CC1CC1)C(=O)N1CC2(CC1C#N)Oc1cc(F)cnc1NC2=O ; CC(NC(=O)C(F)(F)F)C(=O)NC(CC(C)(C)C)C(=O)N1CC2(CC1C#N)Oc1cc(F)cc(F)c1NC2=O ; CC(C)CC(C(=O)N1CC2(CC1C#N)Oc1cc(F)cc(F)c1NC2=O)N(C)C(=O)C(Cc1ccc(F)cc1)NC(=O)C(F)(F)F] 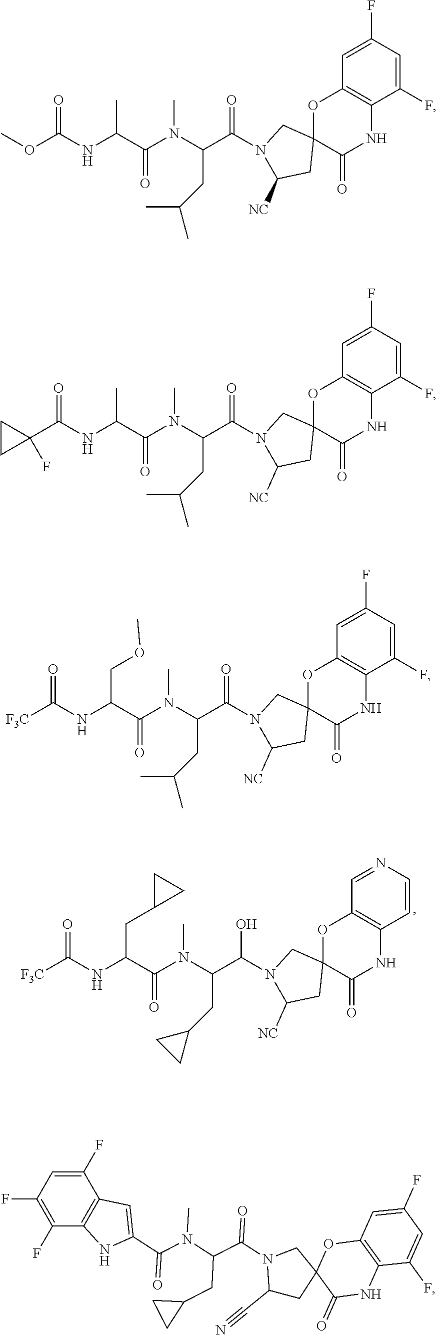 [SMILES: COC(=O)NC(C)C(=O)N(C)C(CC(C)C)C(=O)N1CC2(C[C@H]1C#N)Oc1cc(F)cc(F)c1NC2=O ; CC(C)CC(C(=O)N1CC2(CC1C#N)Oc1cc(F)cc(F)c1NC2=O)N(C)C(=O)C(C)NC(=O)C1(F)CC1 ; COCC(NC(=O)C(F)(F)F)C(=O)N(C)C(CC(C)C)C(=O)N1CC2(CC1C#N)Oc1cc(F)cc(F)c1NC2=O ; CN(C(=O)C(CC1CC1)NC(=O)C(F)(F)F)C(CC1CC1)C(O)N1CC2(CC1C#N)Oc1cnccc1NC2=O ; CN(C(=O)c1cc2c(F)cc(F)c(F)c2[nH]1)C(CC1CC1)C(=O)N1CC2(CC1C#N)Oc1cc(F)cc(F)c1NC2=O] 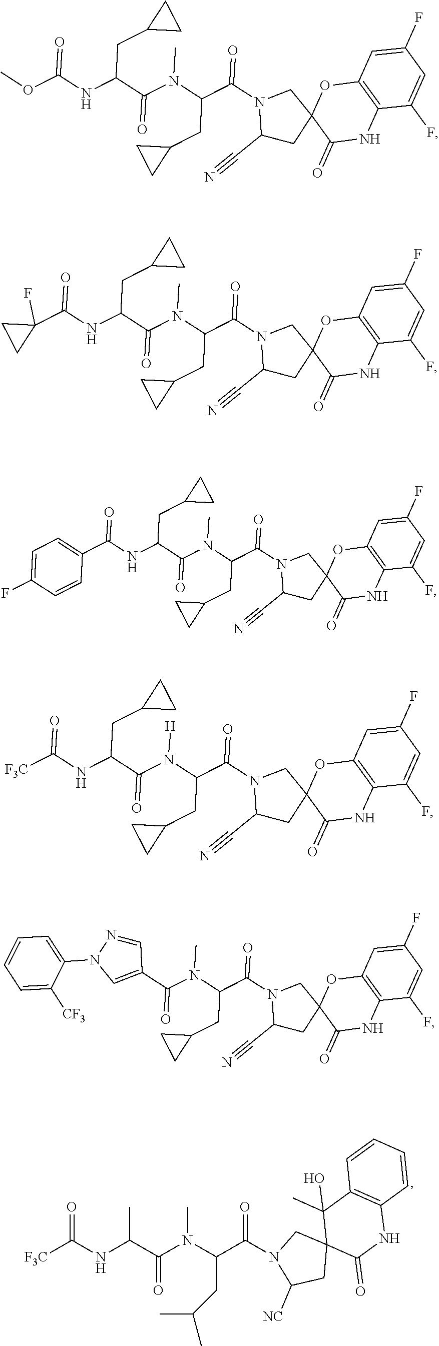 [SMILES: COC(=O)NC(CC1CC1)C(=O)N(C)C(CC1CC1)C(=O)N1CC2(CC1C#N)Oc1cc(F)cc(F)c1NC2=O ; CN(C(=O)C(CC1CC1)NC(=O)C1(F)CC1)C(CC1CC1)C(=O)N1CC2(CC1C#N)Oc1cc(F)cc(F)c1NC2=O ; CN(C(=O)C(CC1CC1)NC(=O)c1ccc(F)cc1)C(CC1CC1)C(=O)N1CC2(CC1C#N)Oc1cc(F)cc(F)c1NC2=O ; N#CC1CC2(CN1C(=O)C(CC1CC1)NC(=O)C(CC1CC1)NC(=O)C(F)(F)F)Oc1cc(F)cc(F)c1NC2=O ; CN(C(=O)c1cnn(-c2ccccc2C(F)(F)F)c1)C(CC1CC1)C(=O)N1CC2(CC1C#N)Oc1cc(F)cc(F)c1NC2=O ; CC(C)CC(C(=O)N1CC2(CC1C#N)C(=O)Nc1ccccc1C2(C)O)N(C)C(=O)C(C)NC(=O)C(F)(F)F] 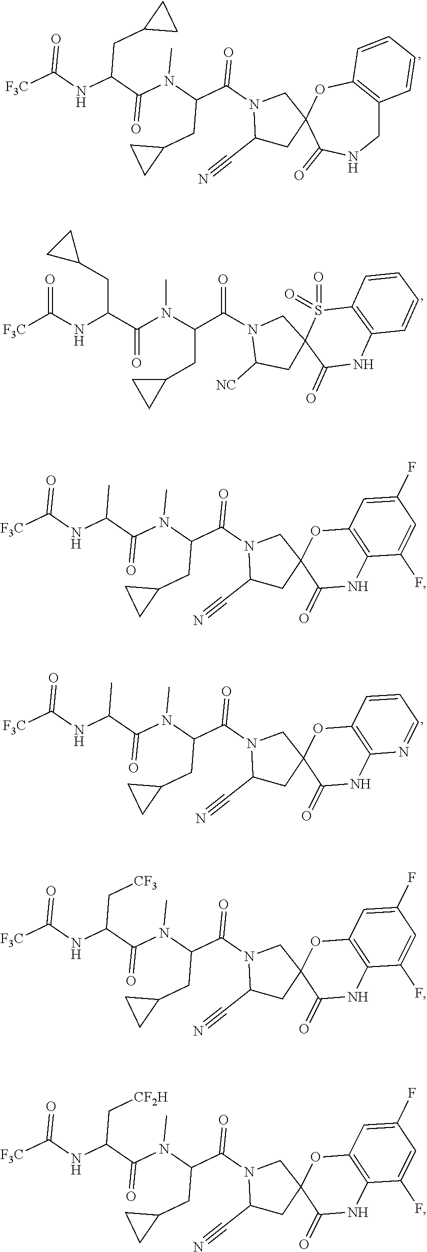 [SMILES: CN(C(=O)C(CC1CC1)NC(=O)C(F)(F)F)C(CC1CC1)C(=O)N1CC2(CC1C#N)Oc1ccccc1CNC2=O ; CN(C(=O)C(CC1CC1)NC(=O)C(F)(F)F)C(CC1CC1)C(=O)N1CC2(CC1C#N)C(=O)Nc1ccccc1S2(=O)=O ; CC(NC(=O)C(F)(F)F)C(=O)N(C)C(CC1CC1)C(=O)N1CC2(CC1C#N)Oc1cc(F)cc(F)c1NC2=O ; CC(NC(=O)C(F)(F)F)C(=O)N(C)C(CC1CC1)C(=O)N1CC2(CC1C#N)Oc1cccnc1NC2=O ; CN(C(=O)C(CC(F)(F)F)NC(=O)C(F)(F)F)C(CC1CC1)C(=O)N1CC2(CC1C#N)Oc1cc(F)cc(F)c1NC2=O ; CN(C(=O)C(CC(F)F)NC(=O)C(F)(F)F)C(CC1CC1)C(=O)N1CC2(CC1C#N)Oc1cc(F)cc(F)c1NC2=O] 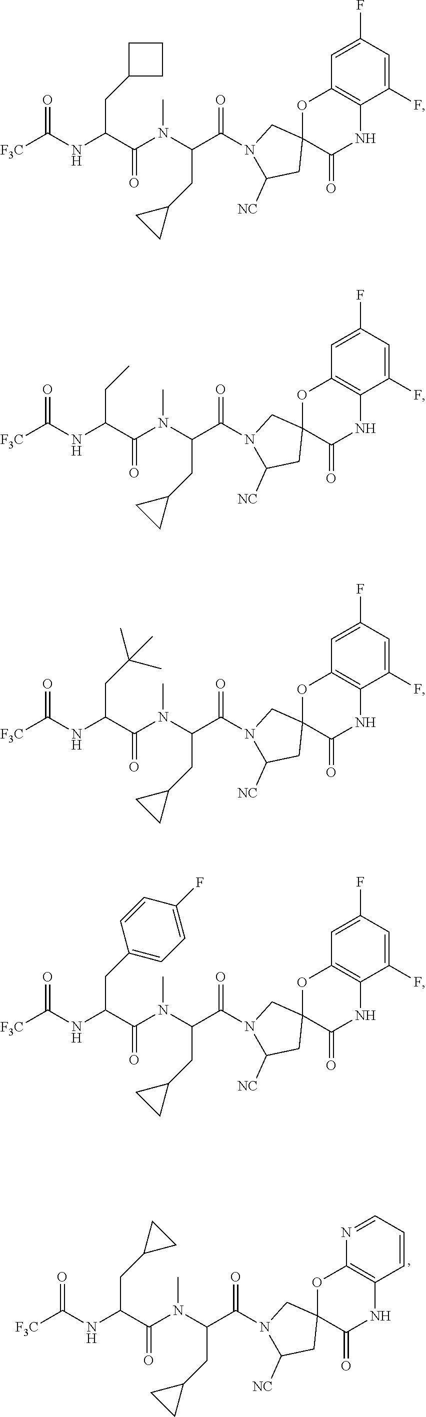 [SMILES: CN(C(=O)C(CC1CCC1)NC(=O)C(F)(F)F)C(CC1CC1)C(=O)N1CC2(CC1C#N)Oc1cc(F)cc(F)c1NC2=O ; CCC(NC(=O)C(F)(F)F)C(=O)N(C)C(CC1CC1)C(=O)N1CC2(CC1C#N)Oc1cc(F)cc(F)c1NC2=O ; CN(C(=O)C(CC(C)(C)C)NC(=O)C(F)(F)F)C(CC1CC1)C(=O)N1CC2(CC1C#N)Oc1cc(F)cc(F)c1NC2=O ; CN(C(=O)C(Cc1ccc(F)cc1)NC(=O)C(F)(F)F)C(CC1CC1)C(=O)N1CC2(CC1C#N)Oc1cc(F)cc(F)c1NC2=O ; CN(C(=O)C(CC1CC1)NC(=O)C(F)(F)F)C(CC1CC1)C(=O)N1CC2(CC1C#N)Oc1ncccc1NC2=O] 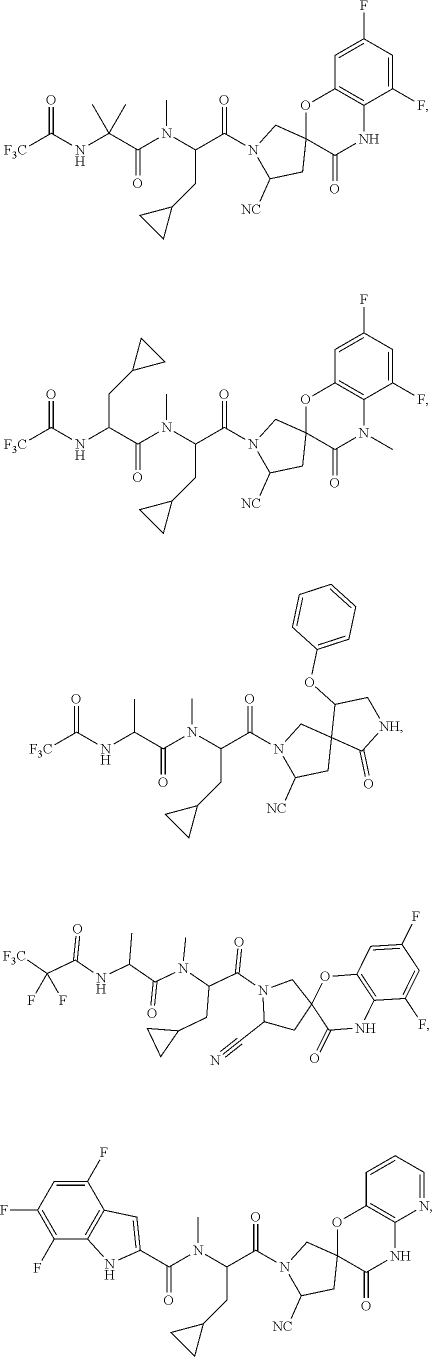 [SMILES: CN(C(=O)C(C)(C)NC(=O)C(F)(F)F)C(CC1CC1)C(=O)N1CC2(CC1C#N)Oc1cc(F)cc(F)c1NC2=O ; CN1C(=O)C2(CC(C#N)N(C(=O)C(CC3CC3)N(C)C(=O)C(CC3CC3)NC(=O)C(F)(F)F)C2)Oc2cc(F)cc(F)c21 ; CC(NC(=O)C(F)(F)F)C(=O)N(C)C(CC1CC1)C(=O)N1CC2(CC1C#N)C(=O)NCC2Oc1ccccc1 ; CC(NC(=O)C(F)(F)C(F)(F)F)C(=O)N(C)C(CC1CC1)C(=O)N1CC2(CC1C#N)Oc1cc(F)cc(F)c1NC2=O ; CN(C(=O)c1cc2c(F)cc(F)c(F)c2[nH]1)C(CC1CC1)C(=O)N1CC2(CC1C#N)Oc1cccnc1NC2=O] 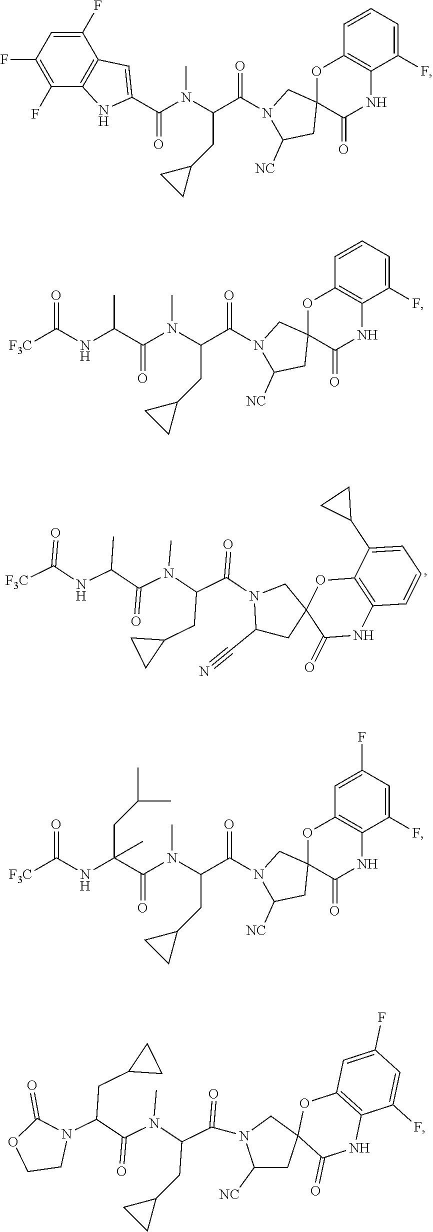 [SMILES: CN(C(=O)c1cc2c(F)cc(F)c(F)c2[nH]1)C(CC1CC1)C(=O)N1CC2(CC1C#N)Oc1cccc(F)c1NC2=O ; CC(NC(=O)C(F)(F)F)C(=O)N(C)C(CC1CC1)C(=O)N1CC2(CC1C#N)Oc1cccc(F)c1NC2=O ; CC(NC(=O)C(F)(F)F)C(=O)N(C)C(CC1CC1)C(=O)N1CC2(CC1C#N)Oc1c(cccc1C1CC1)NC2=O ; CC(C)CC(C)(NC(=O)C(F)(F)F)C(=O)N(C)C(CC1CC1)C(=O)N1CC2(CC1C#N)Oc1cc(F)cc(F)c1NC2=O ; CN(C(=O)C(CC1CC1)N1CCOC1=O)C(CC1CC1)C(=O)N1CC2(CC1C#N)Oc1cc(F)cc(F)c1NC2=O] 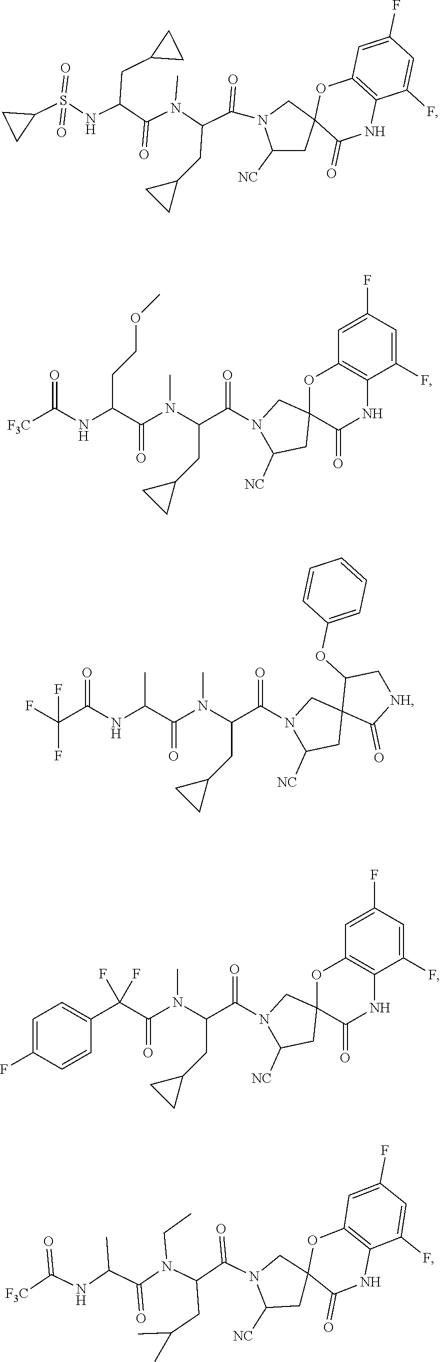 [SMILES: CN(C(=O)C(CC1CC1)NS(=O)(=O)C1CC1)C(CC1CC1)C(=O)N1CC2(CC1C#N)Oc1cc(F)cc(F)c1NC2=O ; COCCC(NC(=O)C(F)(F)F)C(=O)N(C)C(CC1CC1)C(=O)N1CC2(CC1C#N)Oc1cc(F)cc(F)c1NC2=O ; CC(NC(=O)C(F)(F)F)C(=O)N(C)C(CC1CC1)C(=O)N1CC2(CC1C#N)C(=O)NCC2Oc1ccccc1 ; CN(C(=O)C(F)(F)c1ccc(F)cc1)C(CC1CC1)C(=O)N1CC2(CC1C#N)Oc1cc(F)cc(F)c1NC2=O ; CCN(C(=O)C(C)NC(=O)C(F)(F)F)C(CC(C)C)C(=O)N1CC2(CC1C#N)Oc1cc(F)cc(F)c1NC2=O] 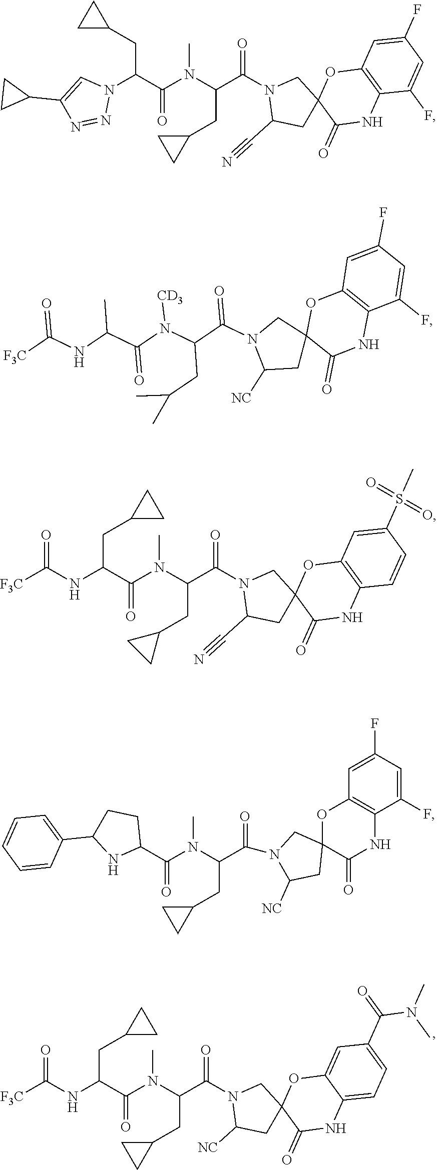 [SMILES: CN(C(=O)C(CC1CC1)n1cc(C2CC2)nn1)C(CC1CC1)C(=O)N1CC2(CC1C#N)Oc1cc(F)cc(F)c1NC2=O ; [2H]C([2H])([2H])N(C(=O)C(C)NC(=O)C(F)(F)F)C(CC(C)C)C(=O)N1CC2(CC1C#N)Oc1cc(F)cc(F)c1NC2=O ; CN(C(=O)C(CC1CC1)NC(=O)C(F)(F)F)C(CC1CC1)C(=O)N1CC2(CC1C#N)Oc1cc(S(C)(=O)=O)ccc1NC2=O ; CN(C(=O)C1CCC(c2ccccc2)N1)C(CC1CC1)C(=O)N1CC2(CC1C#N)Oc1cc(F)cc(F)c1NC2=O ; CN(C)C(=O)c1ccc2c(c1)OC1(CC(C#N)N(C(=O)C(CC3CC3)N(C)C(=O)C(CC3CC3)NC(=O)C(F)(F)F)C1)C(=O)N2] 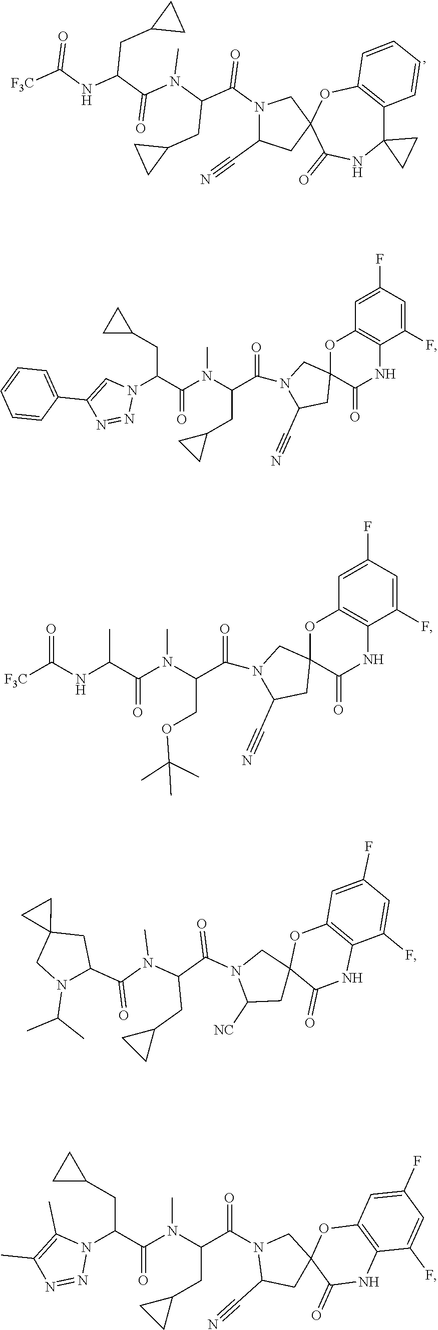 [SMILES: CN(C(=O)C(CC1CC1)NC(=O)C(F)(F)F)C(CC1CC1)C(=O)N1CC2(CC1C#N)Oc1ccccc1C1(CC1)NC2=O ; CN(C(=O)C(CC1CC1)n1cc(-c2ccccc2)nn1)C(CC1CC1)C(=O)N1CC2(CC1C#N)Oc1cc(F)cc(F)c1NC2=O ; CC(NC(=O)C(F)(F)F)C(=O)N(C)C(COC(C)(C)C)C(=O)N1CC2(CC1C#N)Oc1cc(F)cc(F)c1NC2=O ; CC(C)N1CC2(CC2)CC1C(=O)N(C)C(CC1CC1)C(=O)N1CC2(CC1C#N)Oc1cc(F)cc(F)c1NC2=O ; Cc1nnn(C(CC2CC2)C(=O)N(C)C(CC2CC2)C(=O)N2CC3(CC2C#N)Oc2cc(F)cc(F)c2NC3=O)c1C] 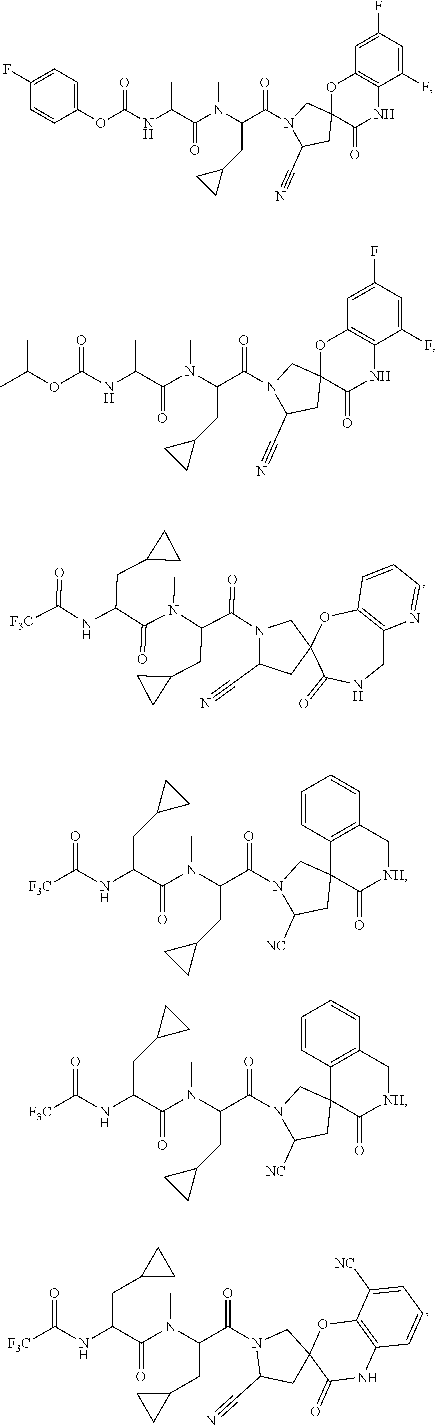 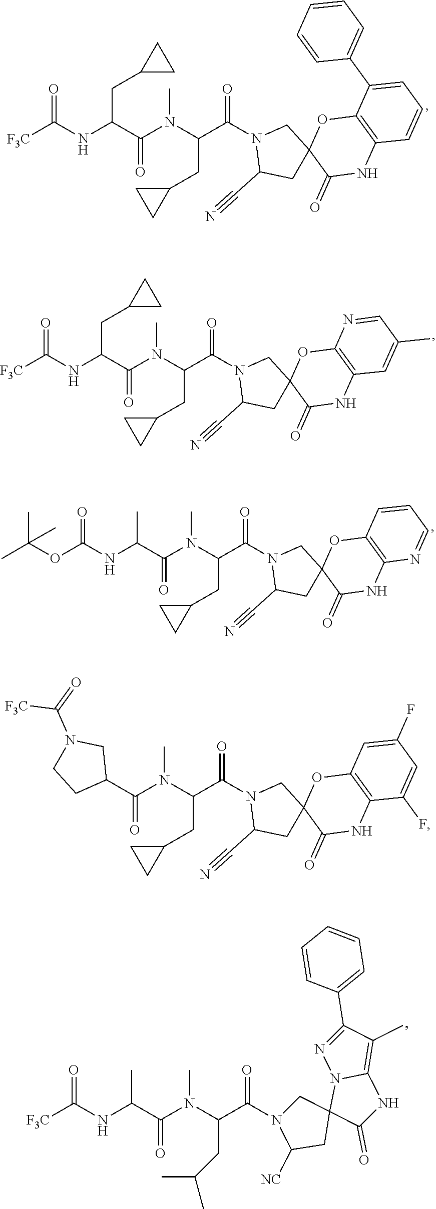 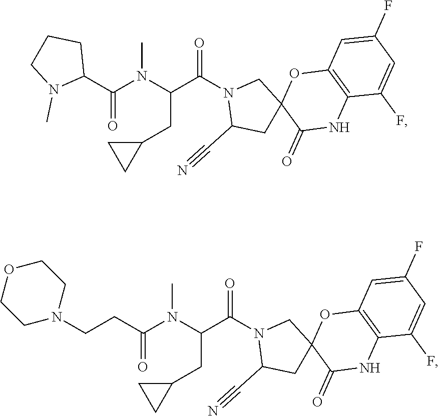 [SMILES: CN1CCCC1C(=O)N(C)C(CC1CC1)C(=O)N1CC2(CC1C#N)Oc1cc(F)cc(F)c1NC2=O ; CN(C(=O)CCN1CCOCC1)C(CC1CC1)C(=O)N1CC2(CC1C#N)Oc1cc(F)cc(F)c1NC2=O] 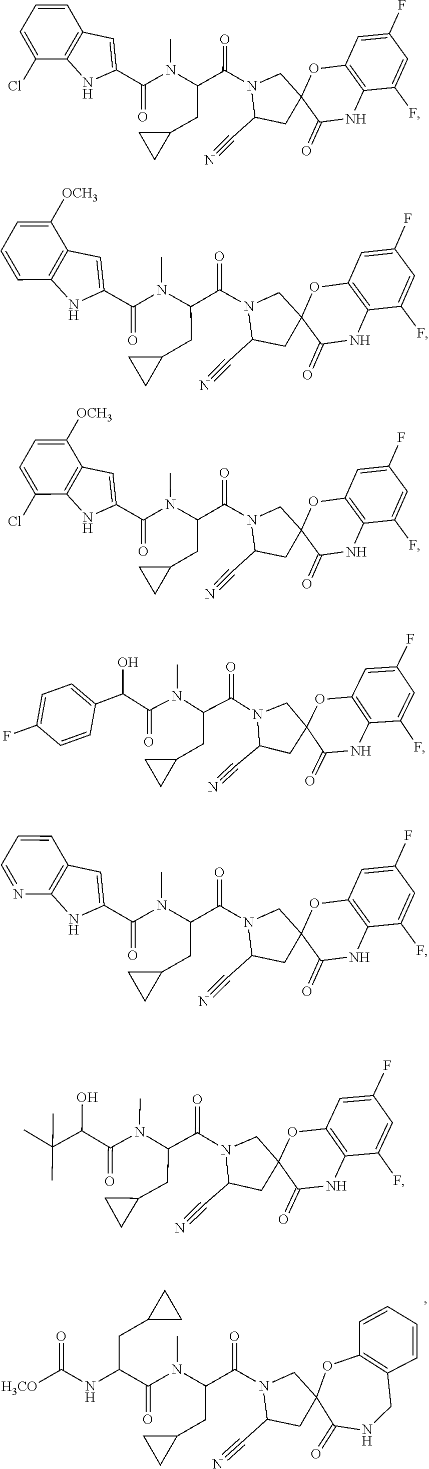 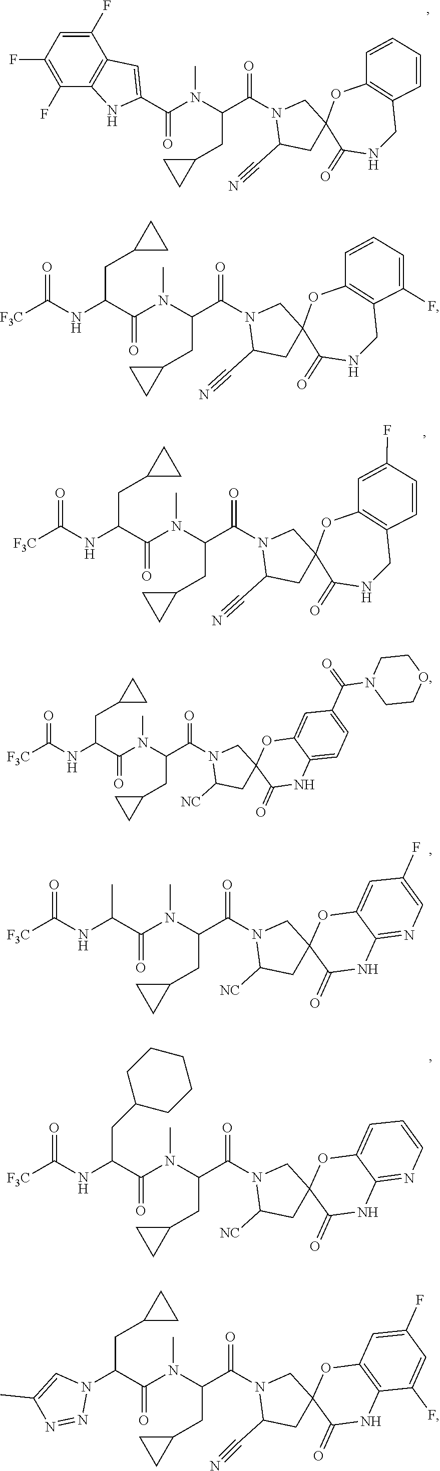 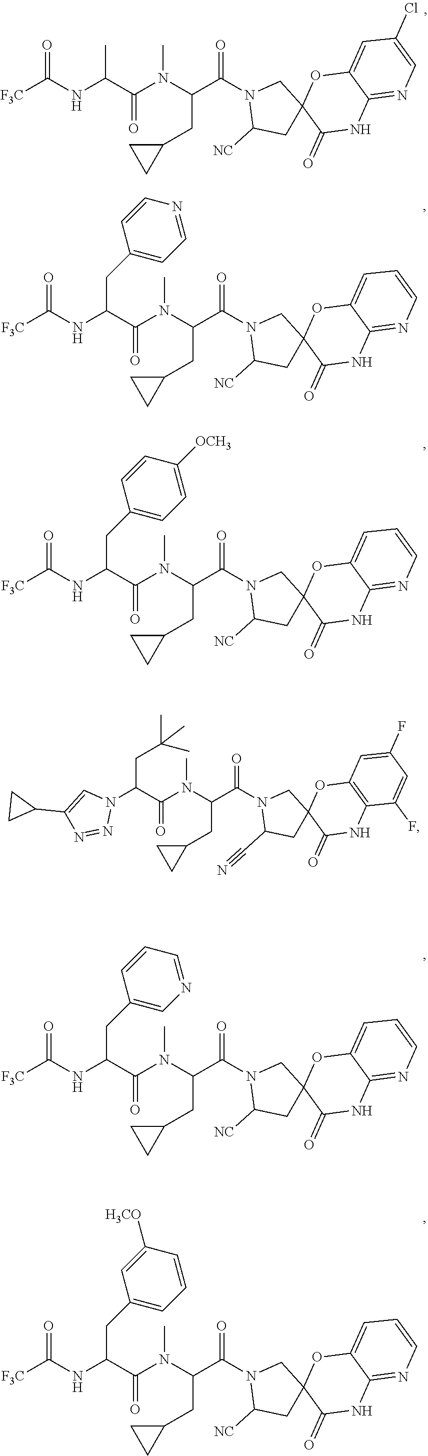 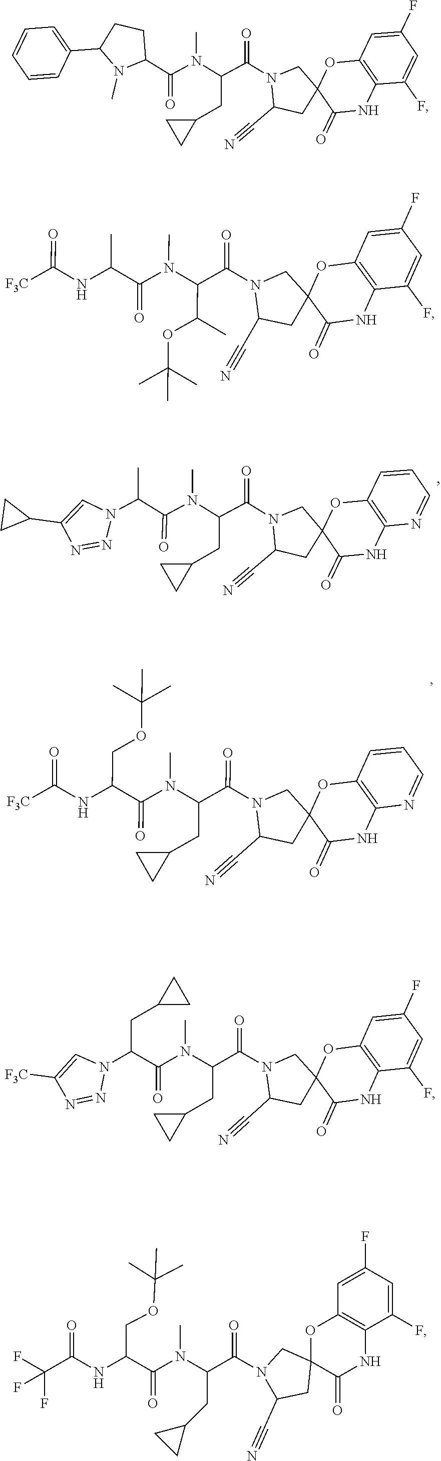 [SMILES: CN(C(=O)C1CCC(c2ccccc2)N1C)C(CC1CC1)C(=O)N1CC2(CC1C#N)Oc1cc(F)cc(F)c1NC2=O ; CC(NC(=O)C(F)(F)F)C(=O)N(C)C(C(=O)N1CC2(CC1C#N)Oc1cc(F)cc(F)c1NC2=O)C(C)OC(C)(C)C ; CC(C(=O)N(C)C(CC1CC1)C(=O)N1CC2(CC1C#N)Oc1cccnc1NC2=O)n1cc(C2CC2)nn1 ; CN(C(=O)C(COC(C)(C)C)NC(=O)C(F)(F)F)C(CC1CC1)C(=O)N1CC2(CC1C#N)Oc1cccnc1NC2=O ; CN(C(=O)C(CC1CC1)n1cc(C(F)(F)F)nn1)C(CC1CC1)C(=O)N1CC2(CC1C#N)Oc1cc(F)cc(F)c1NC2=O ; CN(C(=O)C(COC(C)(C)C)NC(=O)C(F)(F)F)C(CC1CC1)C(=O)N1CC2(CC1C#N)Oc1cc(F)cc(F)c1NC2=O] 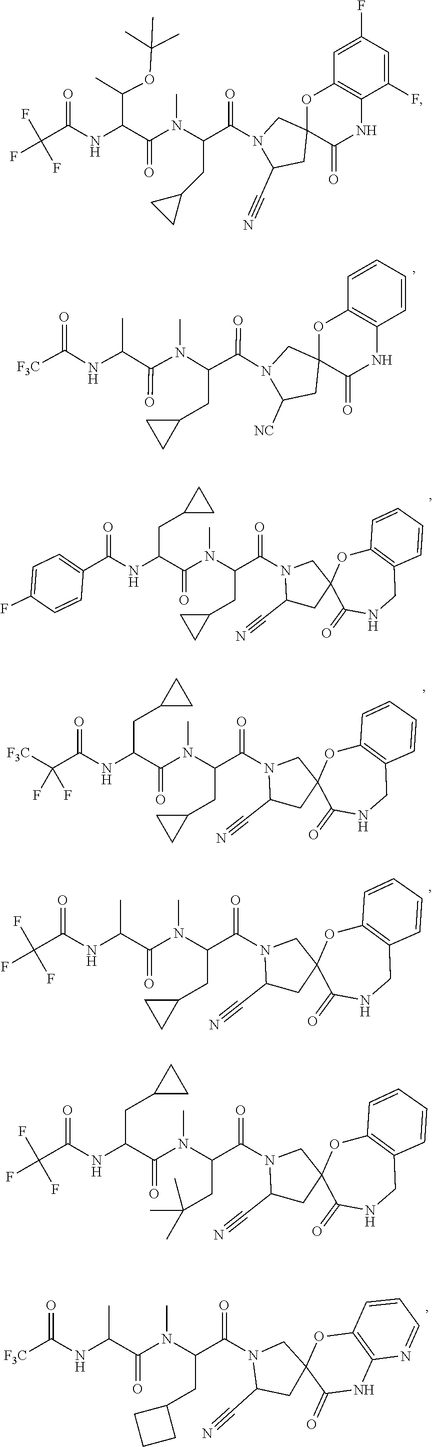 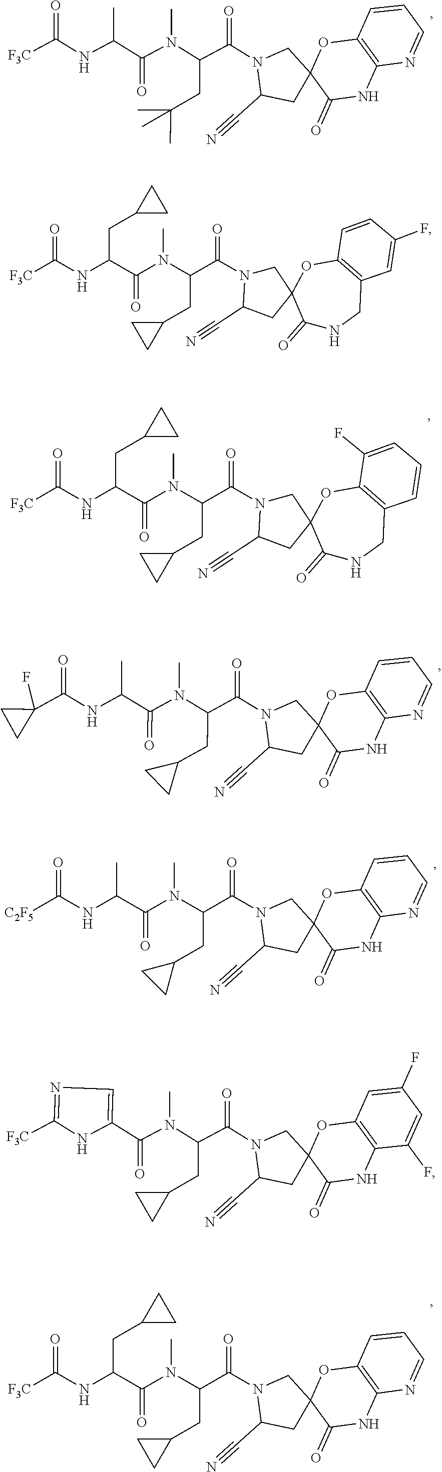 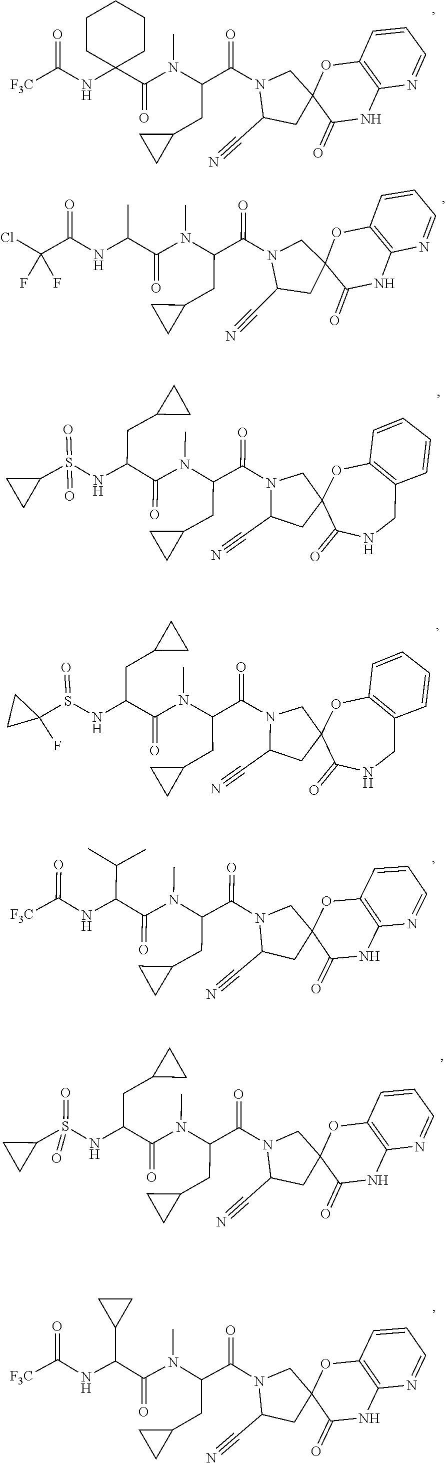 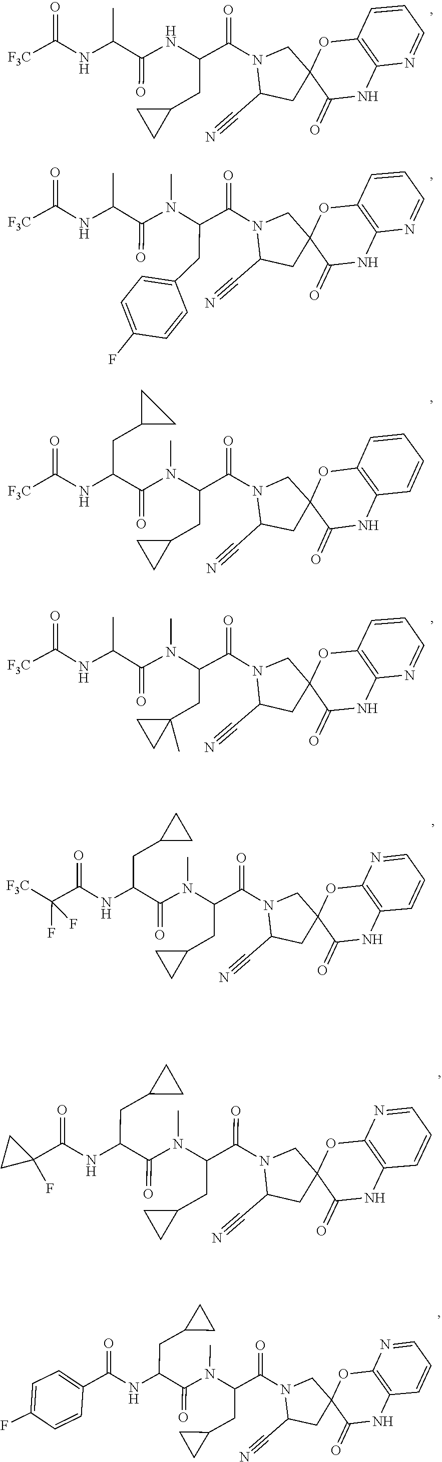 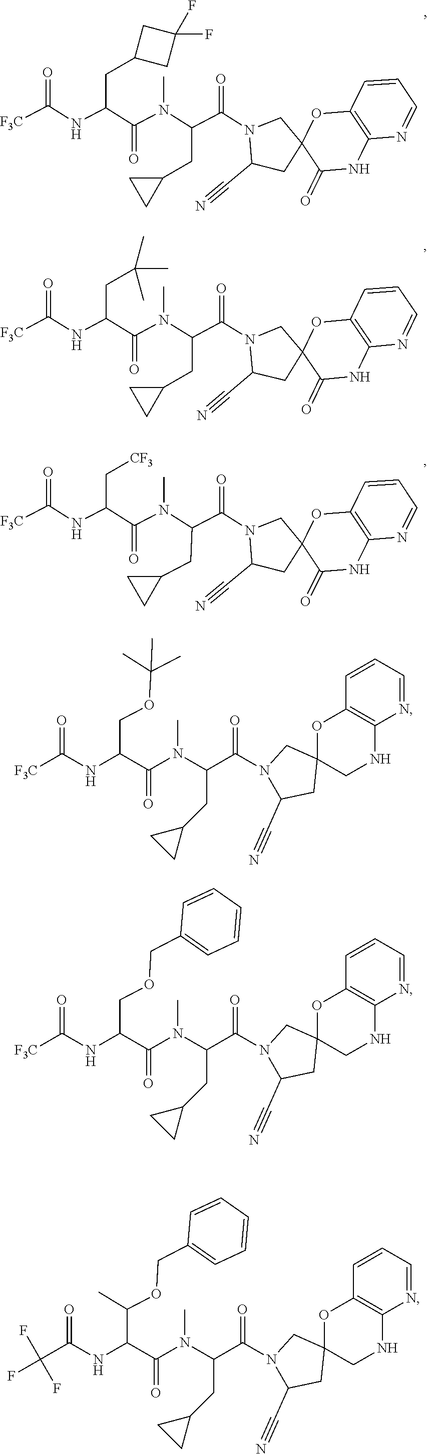 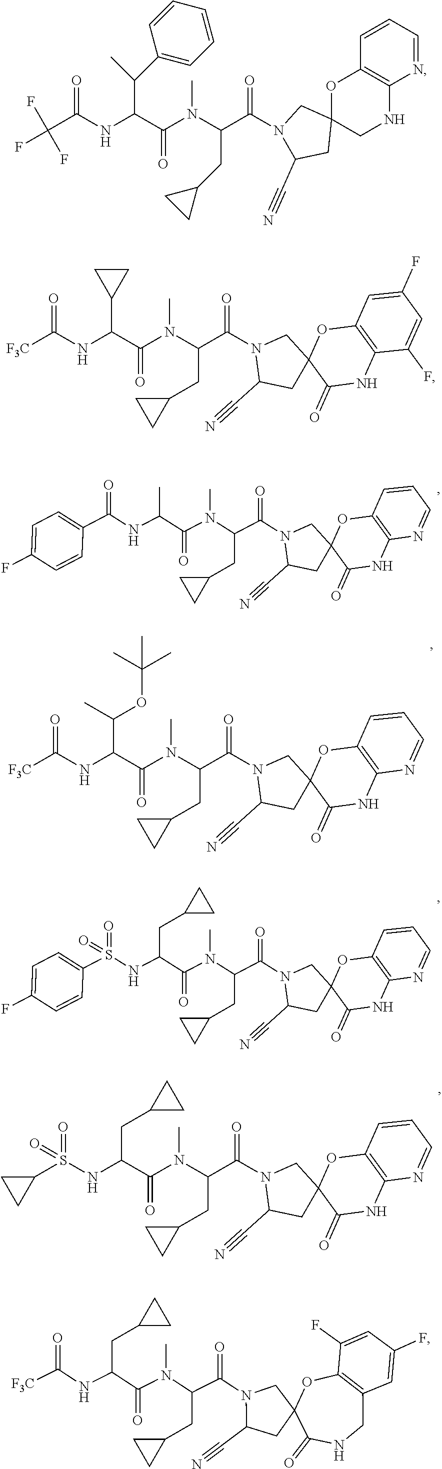 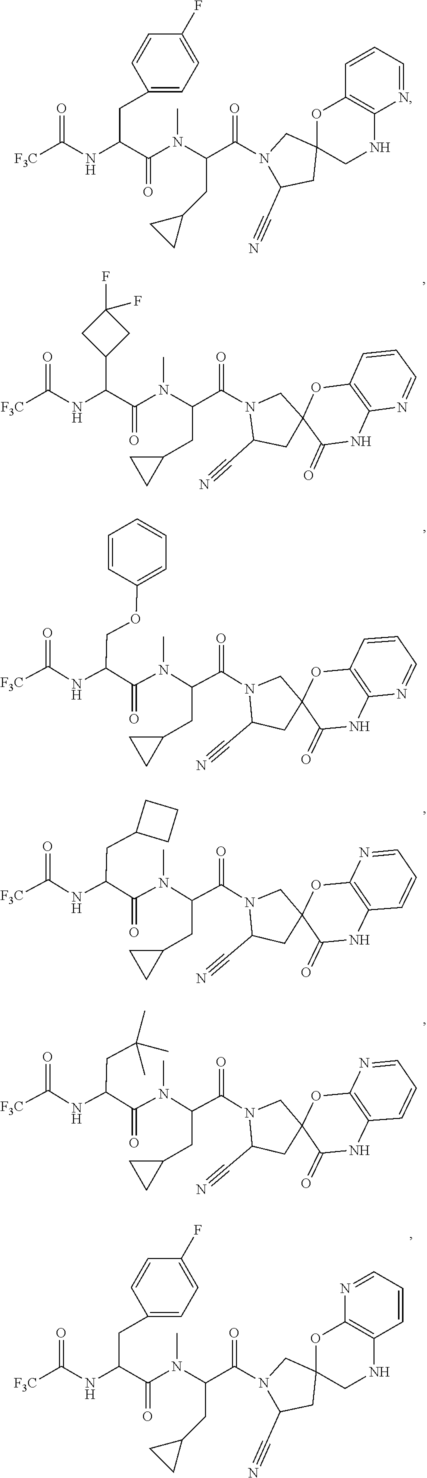 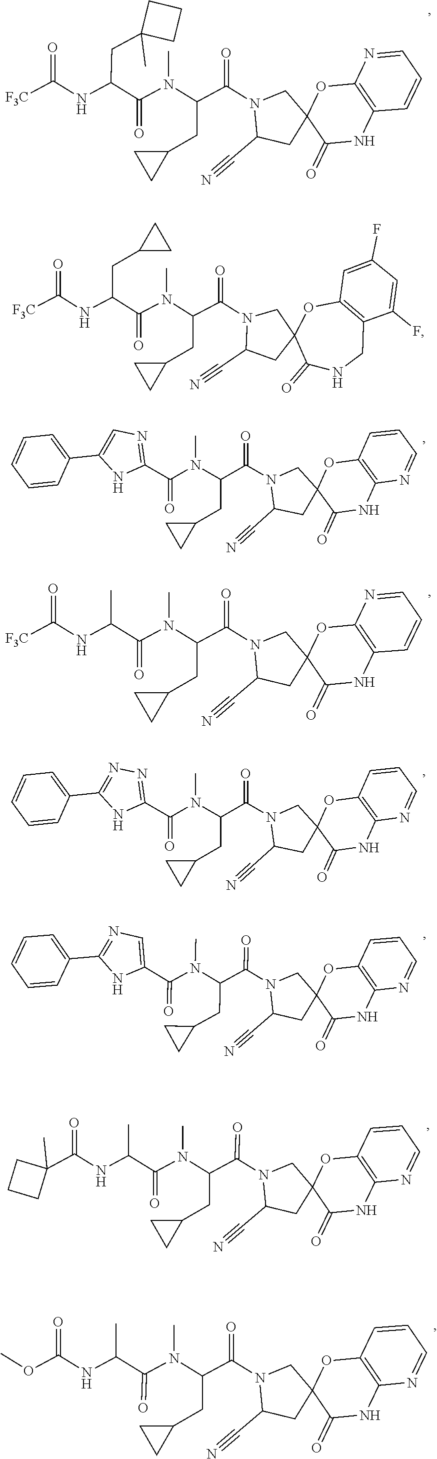 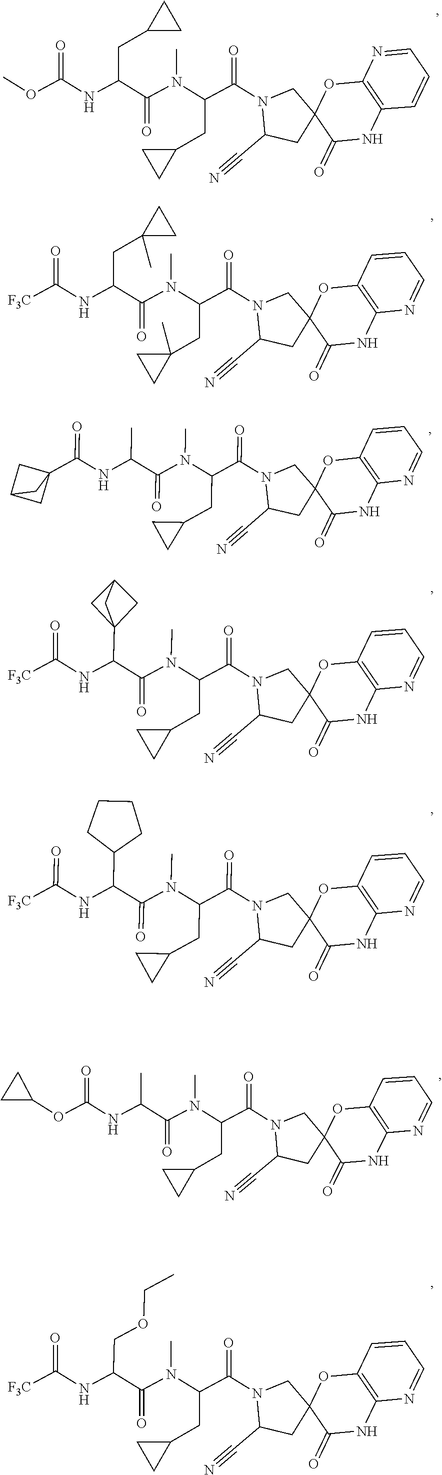 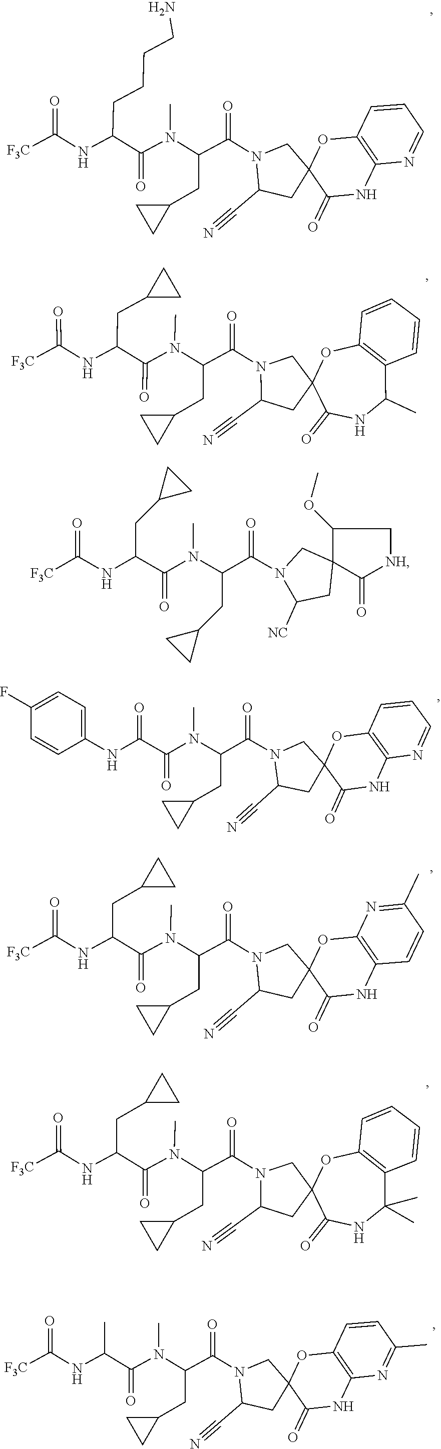 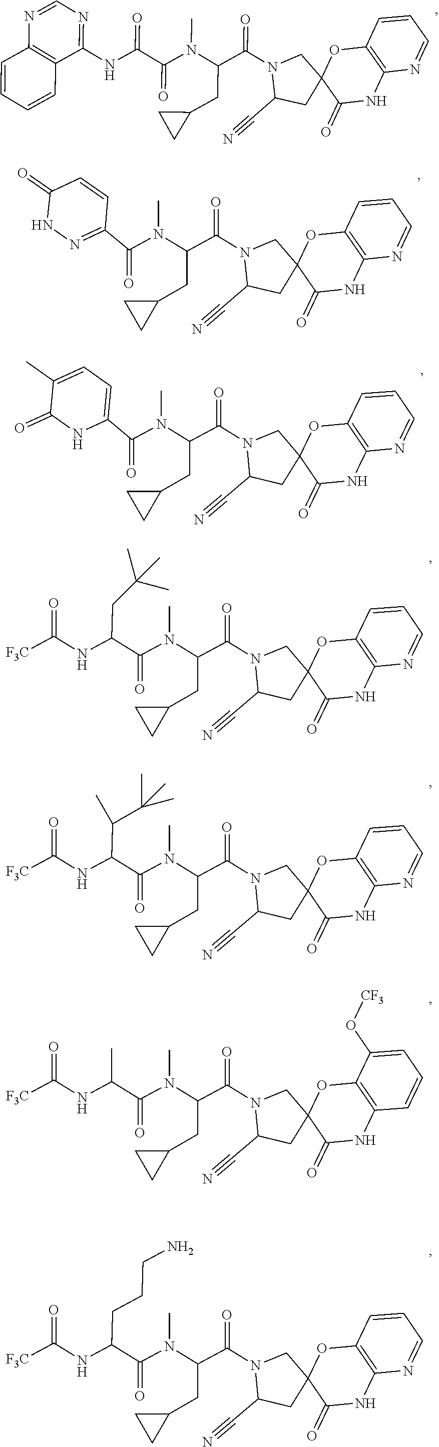 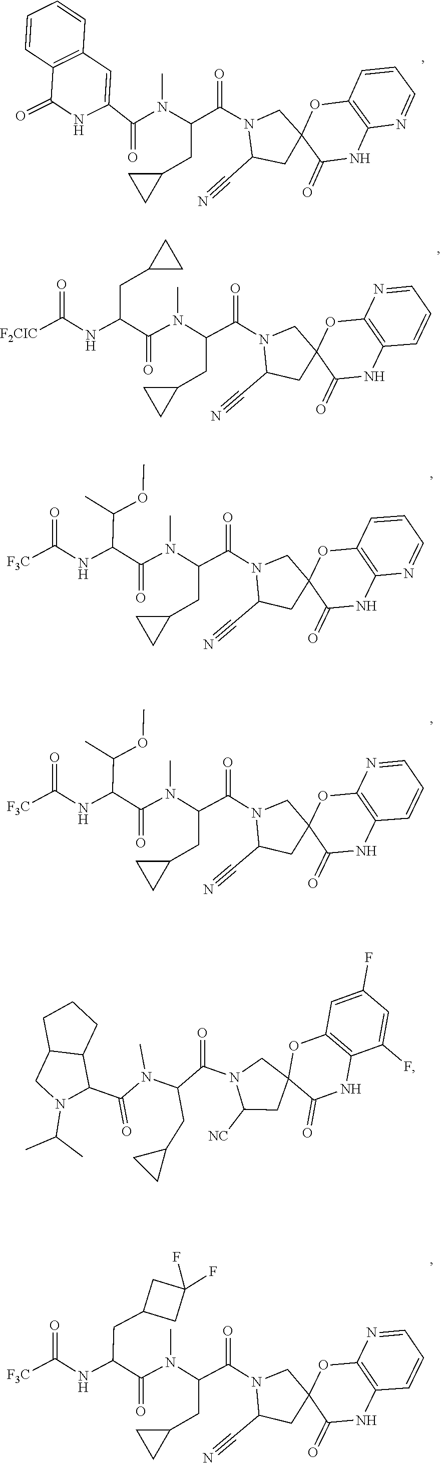 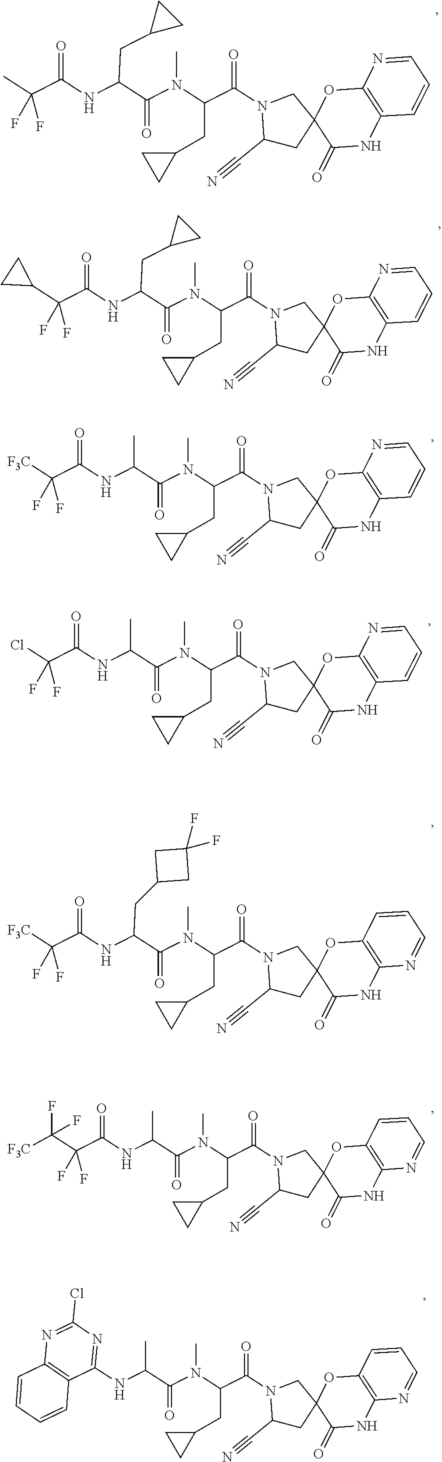 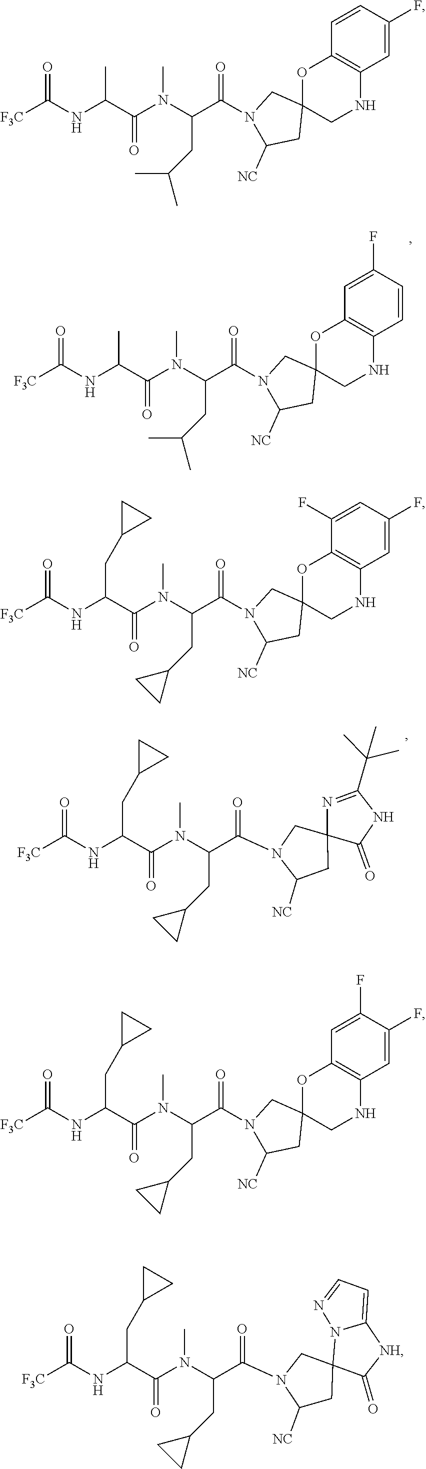 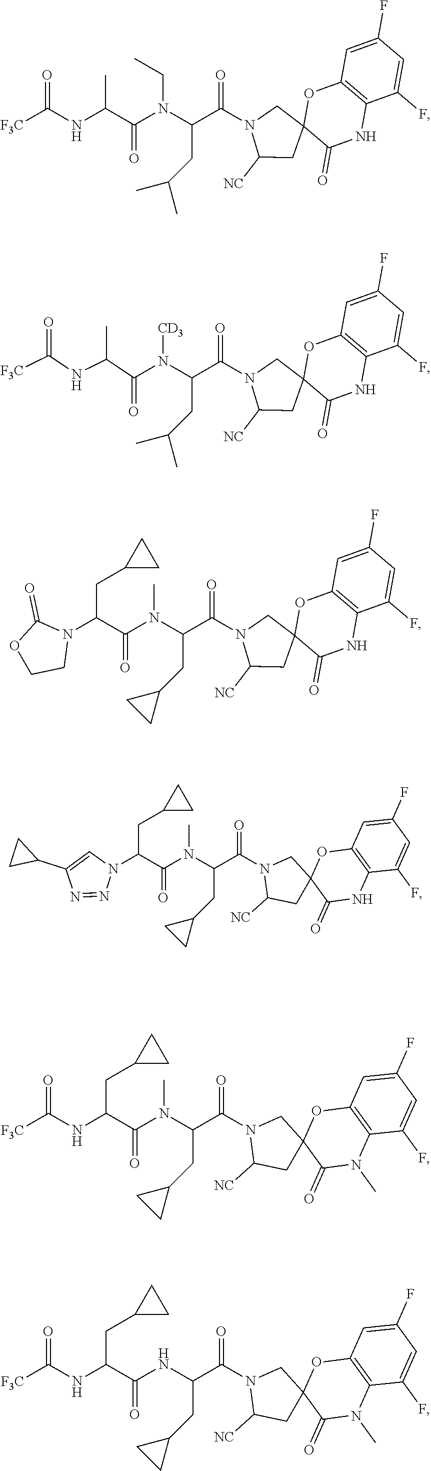 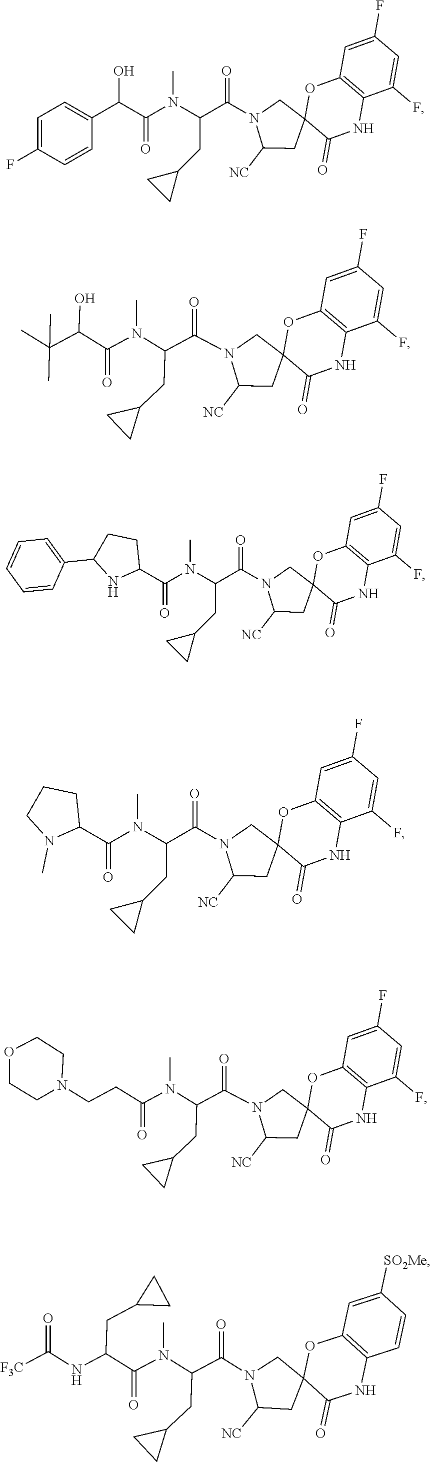 [SMILES: CN(C(=O)C(O)c1ccc(F)cc1)C(CC1CC1)C(=O)N1CC2(CC1C#N)Oc1cc(F)cc(F)c1NC2=O ; CN(C(=O)C(O)C(C)(C)C)C(CC1CC1)C(=O)N1CC2(CC1C#N)Oc1cc(F)cc(F)c1NC2=O ; CN(C(=O)C1CCC(c2ccccc2)N1)C(CC1CC1)C(=O)N1CC2(CC1C#N)Oc1cc(F)cc(F)c1NC2=O ; CN1CCCC1C(=O)N(C)C(CC1CC1)C(=O)N1CC2(CC1C#N)Oc1cc(F)cc(F)c1NC2=O ; CN(C(=O)CCN1CCOCC1)C(CC1CC1)C(=O)N1CC2(CC1C#N)Oc1cc(F)cc(F)c1NC2=O ; CN(C(=O)C(CC1CC1)NC(=O)C(F)(F)F)C(CC1CC1)C(=O)N1CC2(CC1C#N)Oc1cc(S(C)(=O)=O)ccc1NC2=O] 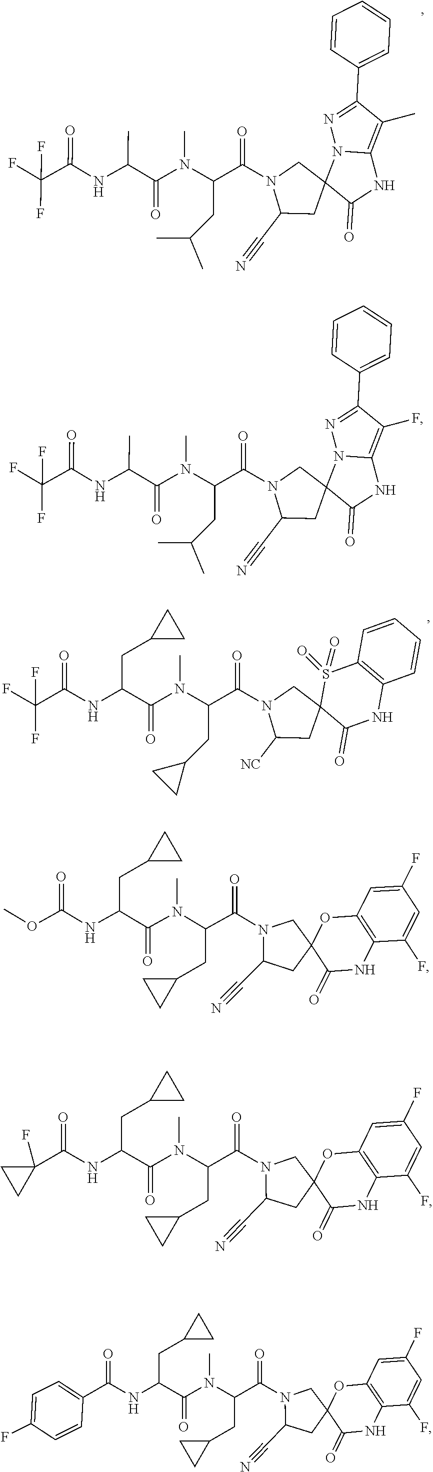 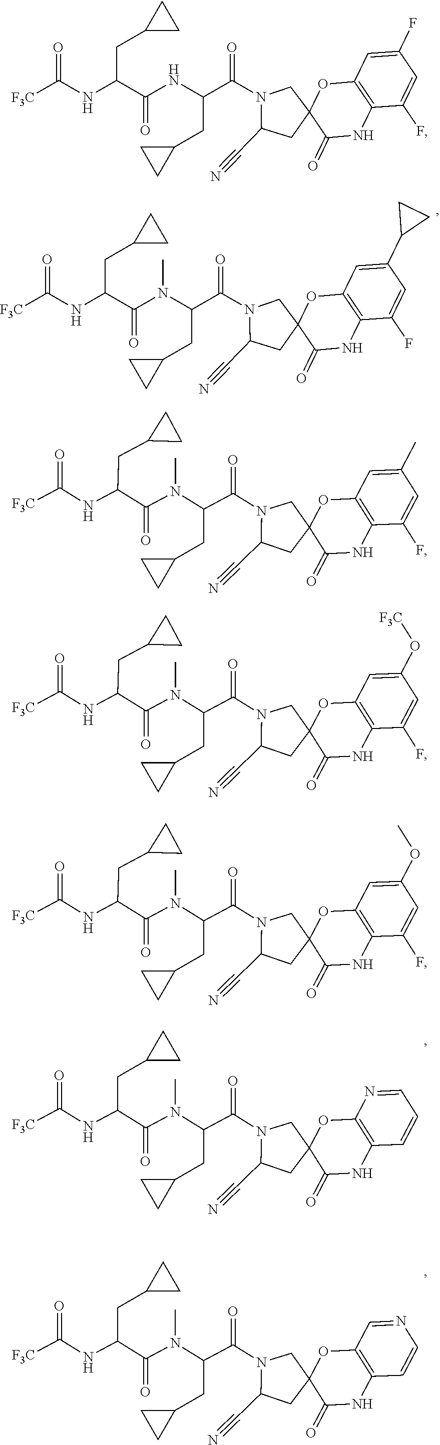 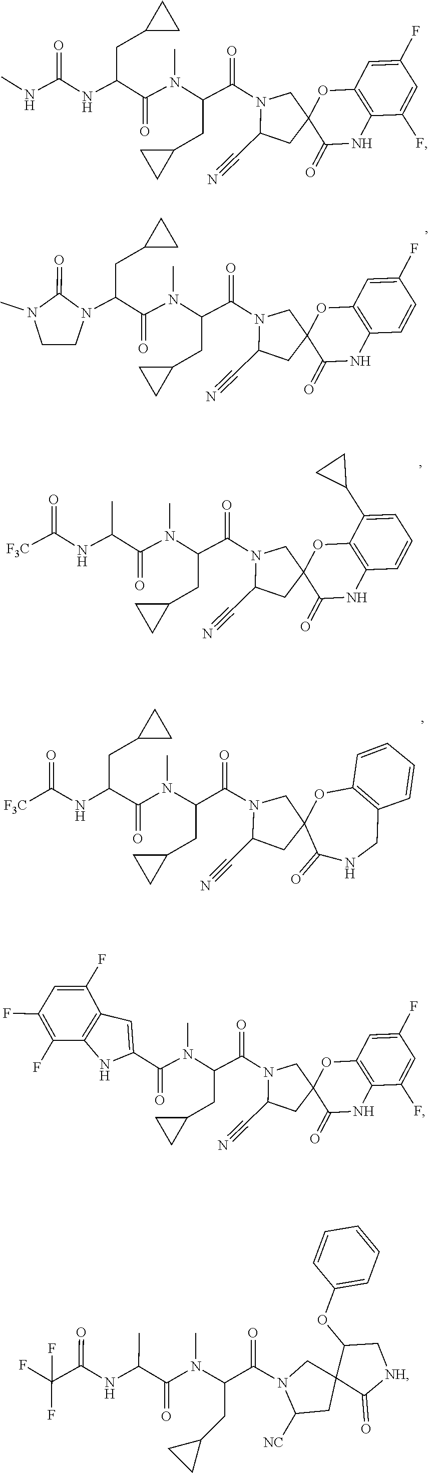 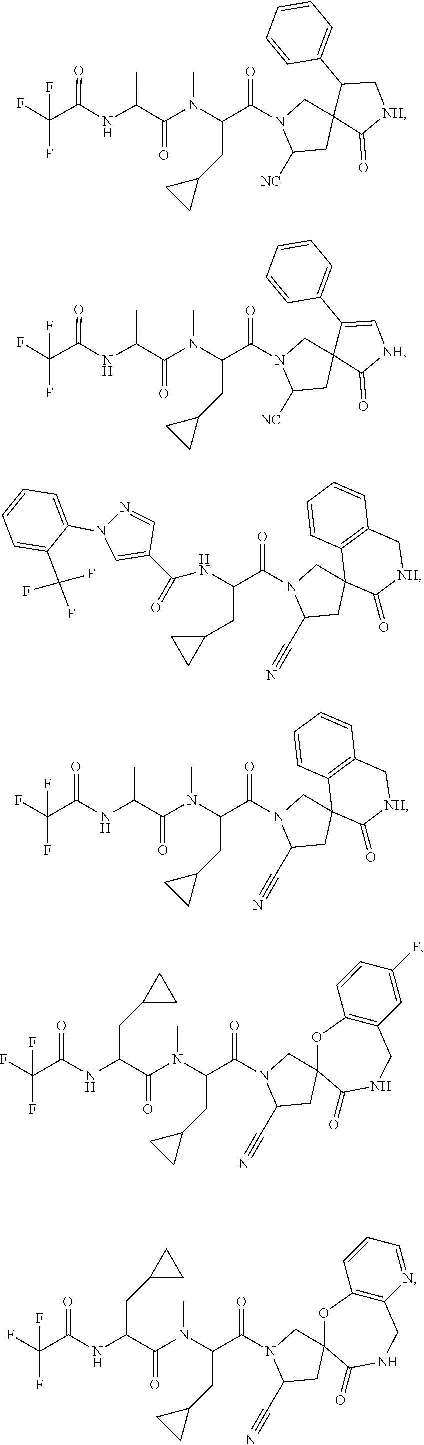 [SMILES: CC(NC(=O)C(F)(F)F)C(=O)N(C)C(CC1CC1)C(=O)N1CC2(CC1C#N)C(=O)NCC2c1ccccc1 ; CC(NC(=O)C(F)(F)F)C(=O)N(C)C(CC1CC1)C(=O)N1CC2(CC1C#N)C(=O)NC=C2c1ccccc1 ; N#CC1CC2(CN1C(=O)C(CC1CC1)NC(=O)c1cnn(-c3ccccc3C(F)(F)F)c1)C(=O)NCc1ccccc12 ; CC(NC(=O)C(F)(F)F)C(=O)N(C)C(CC1CC1)C(=O)N1CC2(CC1C#N)C(=O)NCc1ccccc12 ; CN(C(=O)C(CC1CC1)NC(=O)C(F)(F)F)C(CC1CC1)C(=O)N1CC2(CC1C#N)Oc1ccc(F)cc1CNC2=O ; CN(C(=O)C(CC1CC1)NC(=O)C(F)(F)F)C(CC1CC1)C(=O)N1CC2(CC1C#N)Oc1cccnc1CNC2=O] 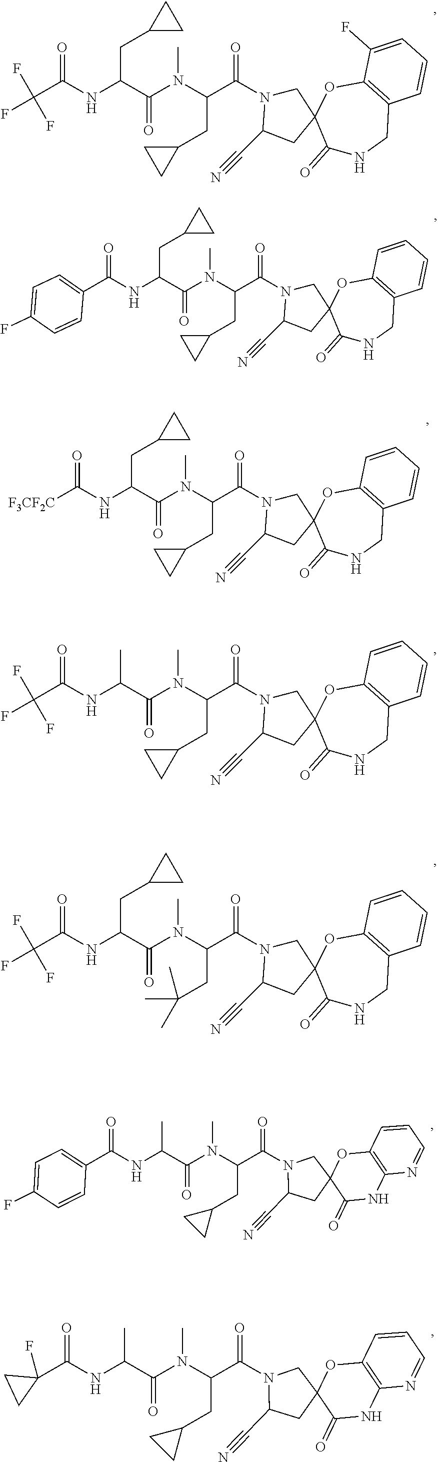 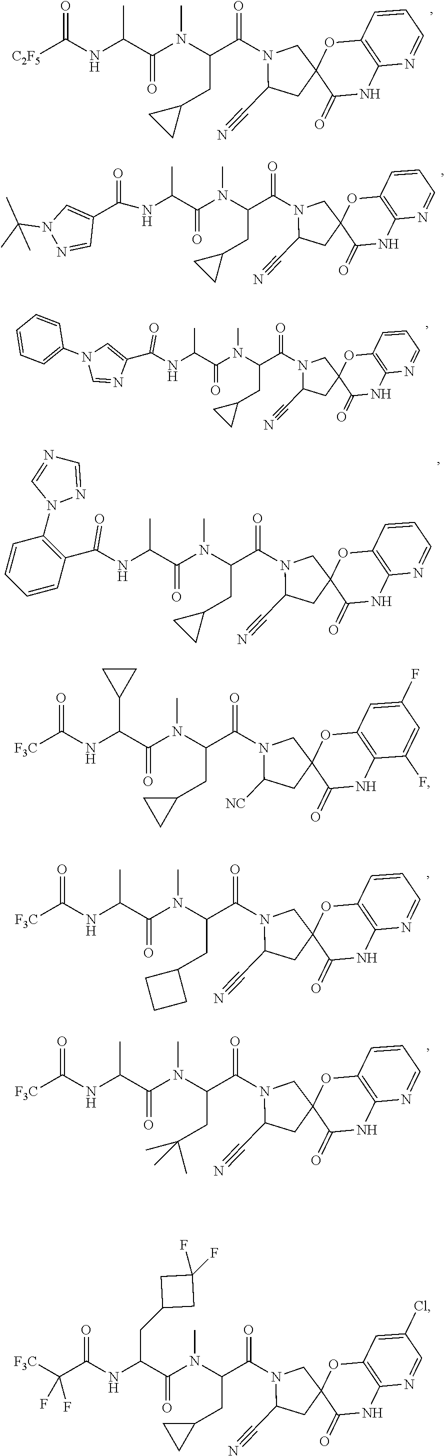 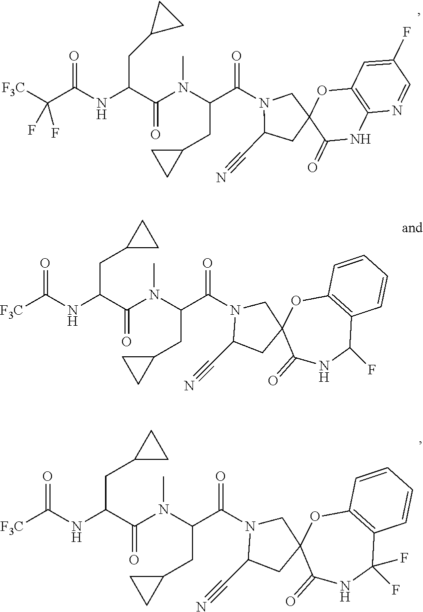 [SMILES: CN(C(=O)C(CC1CC1)NC(=O)C(F)(F)C(F)(F)F)C(CC1CC1)C(=O)N1CC2(CC1C#N)Oc1cc(F)cnc1NC2=O ; CN(C(=O)C(CC1CC1)NC(=O)C(F)(F)F)C(CC1CC1)C(=O)N1CC2(CC1C#N)Oc1ccccc1C(F)NC2=O ; CN(C(=O)C(CC1CC1)NC(=O)C(F)(F)F)C(CC1CC1)C(=O)N1CC2(CC1C#N)Oc1ccccc1C(F)(F)NC2=O]
or a pharmaceutically acceptable salt of any of the foregoing.

124. The compound of claim 1, wherein the compound is selected from the group consisting of: 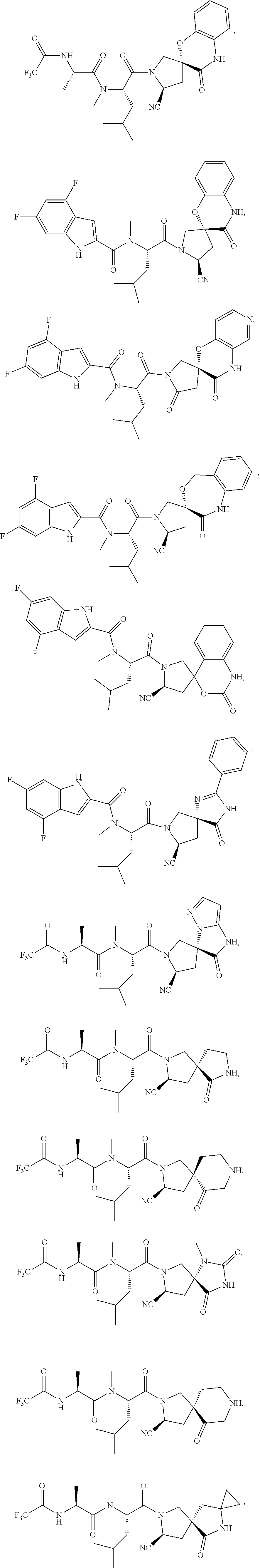 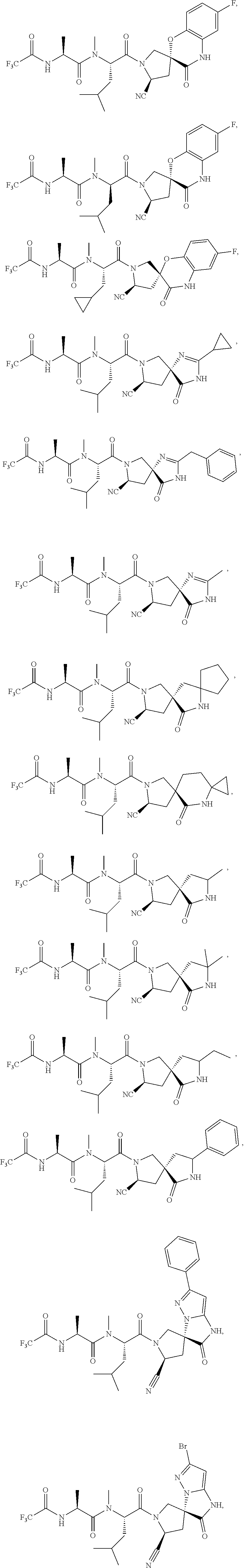 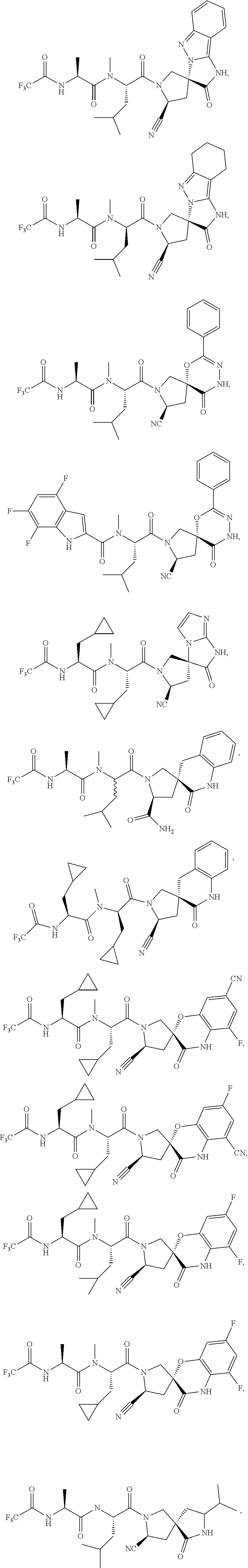 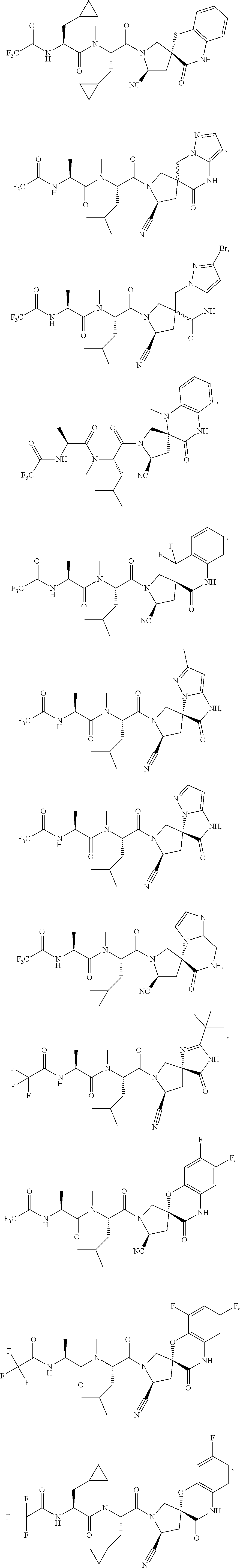 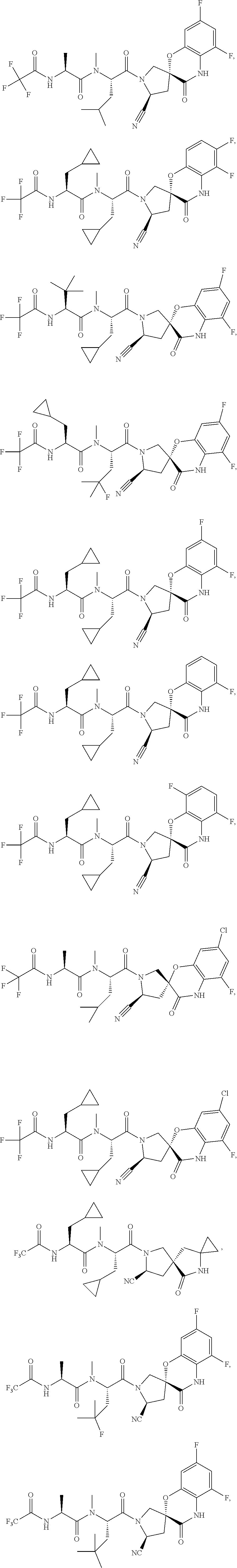 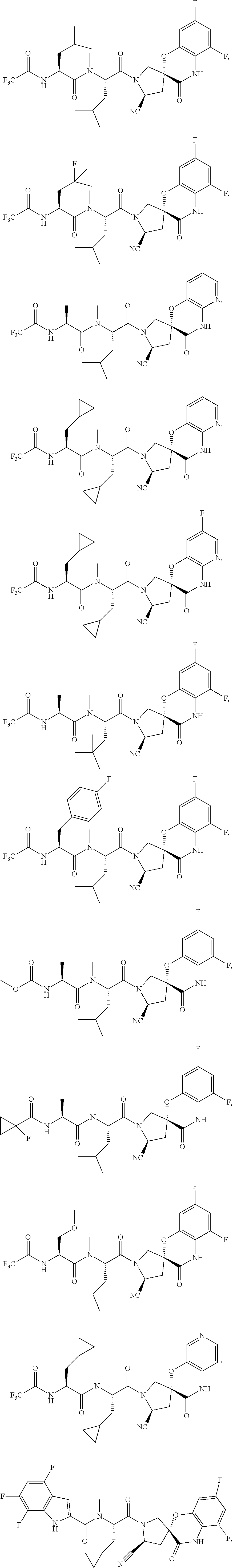 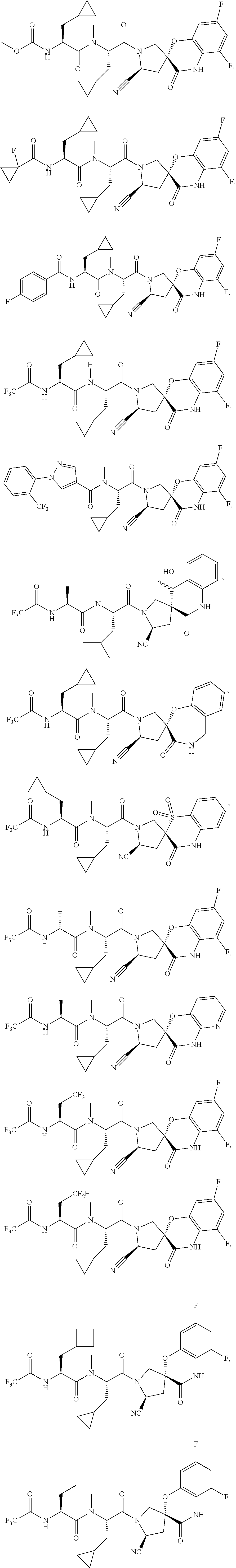 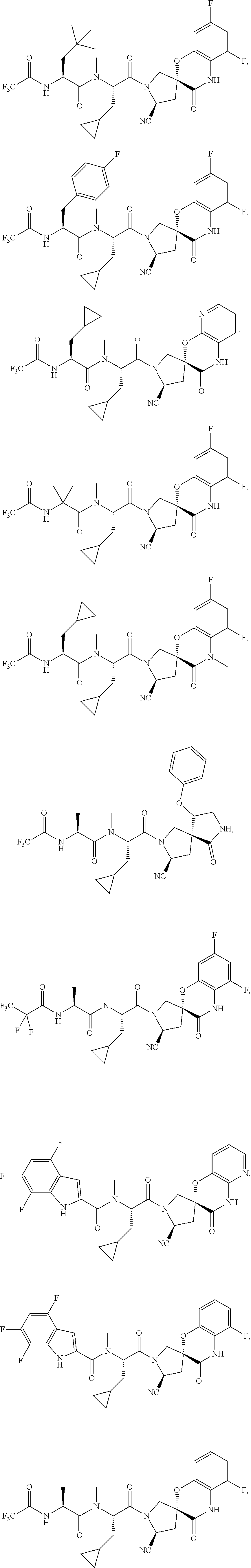 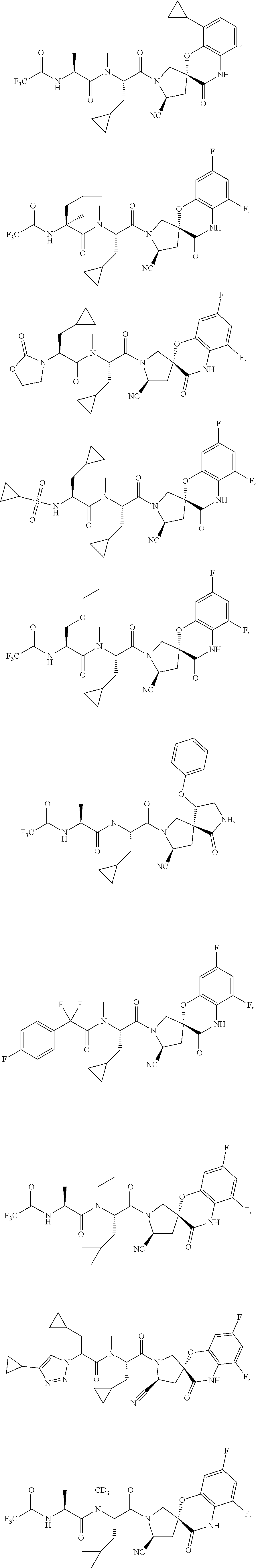 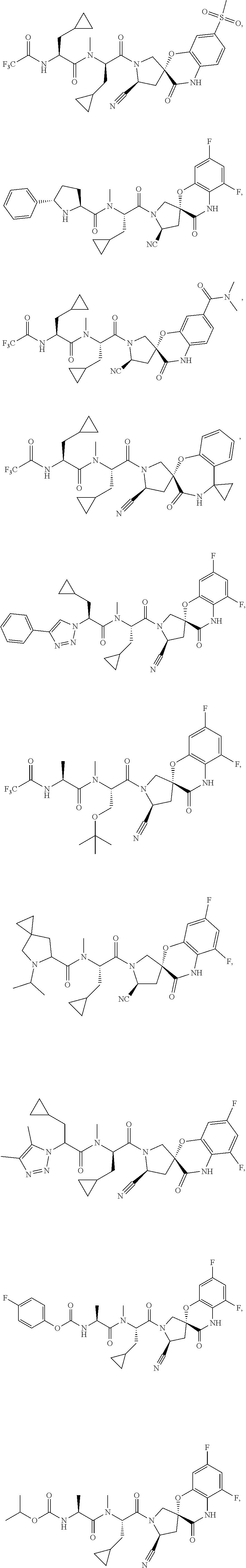 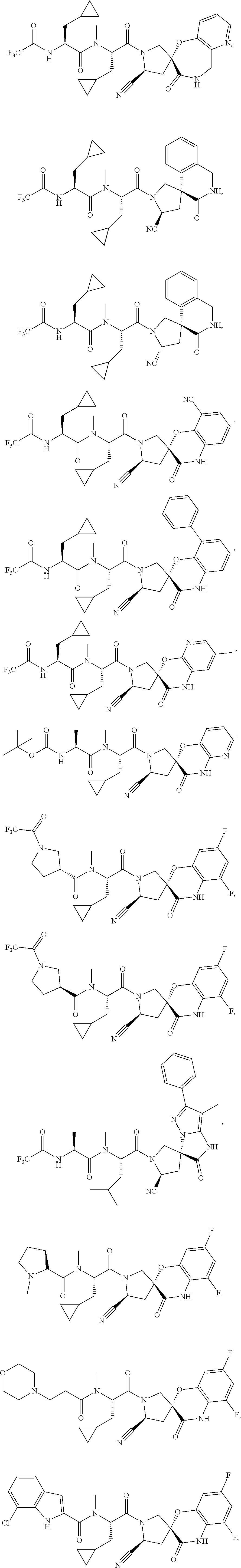 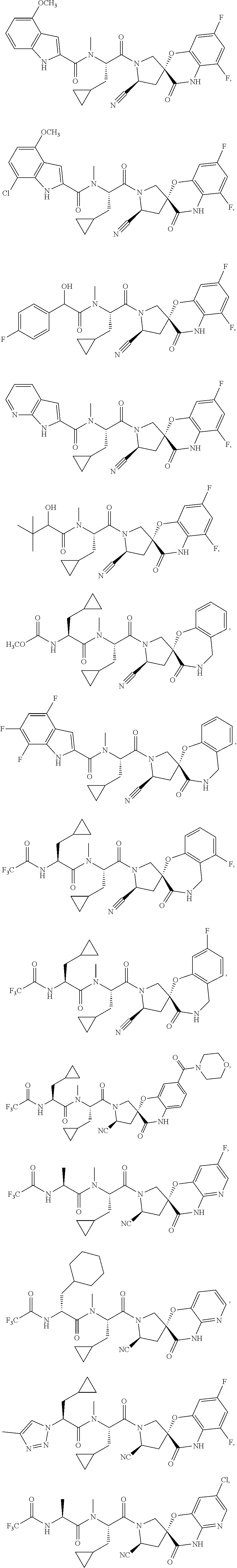 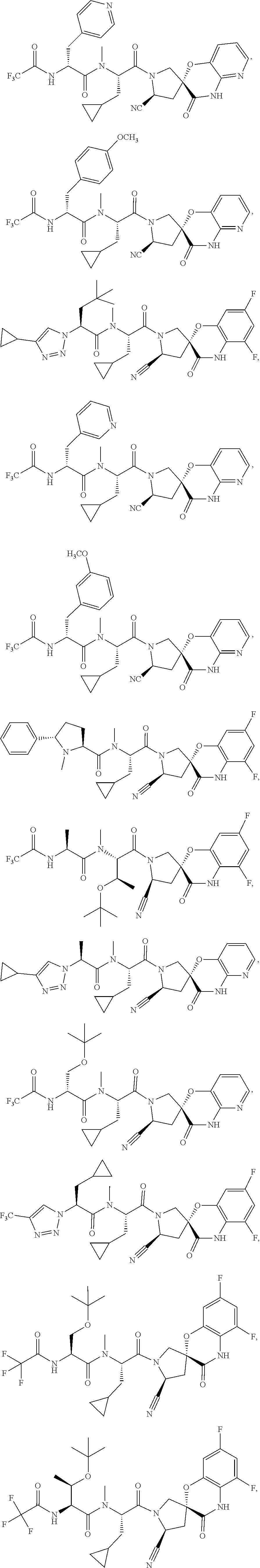 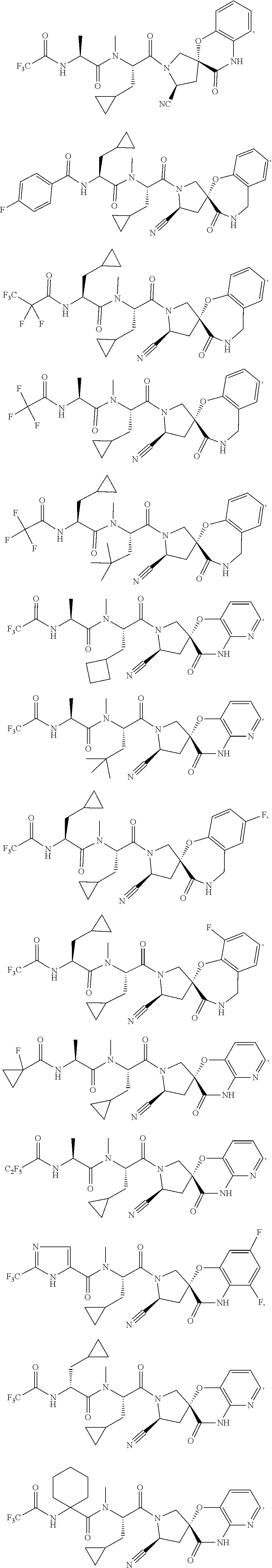 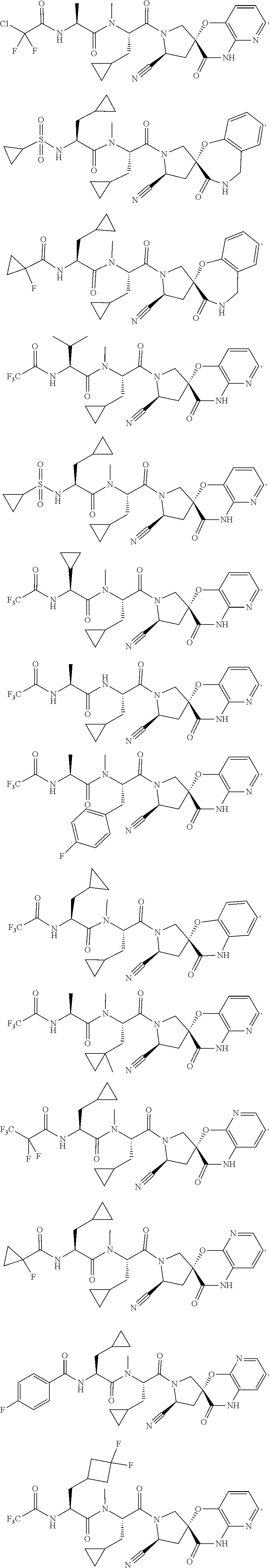 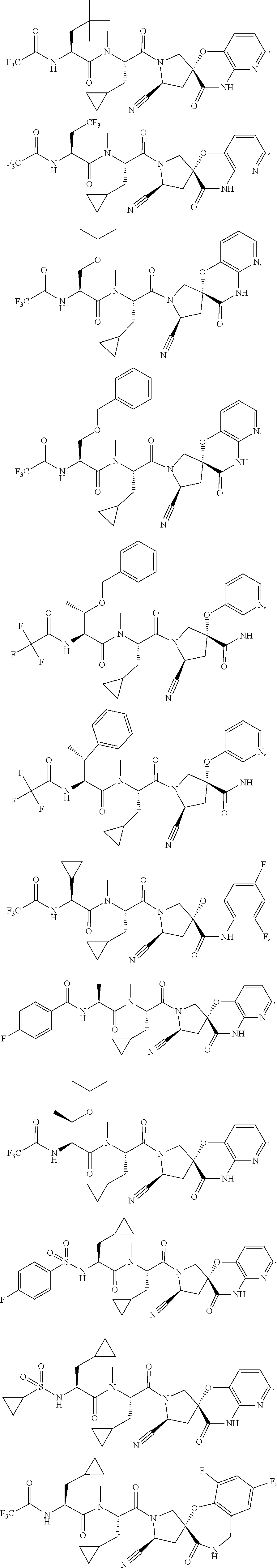 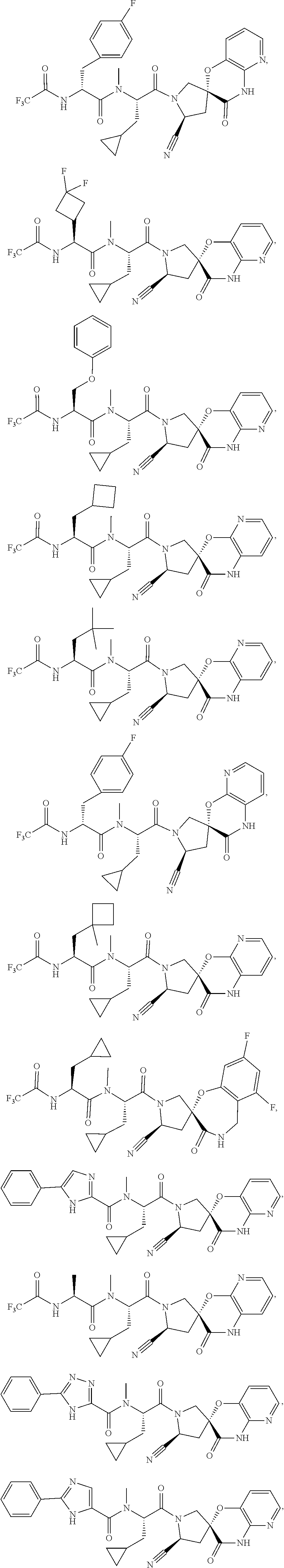 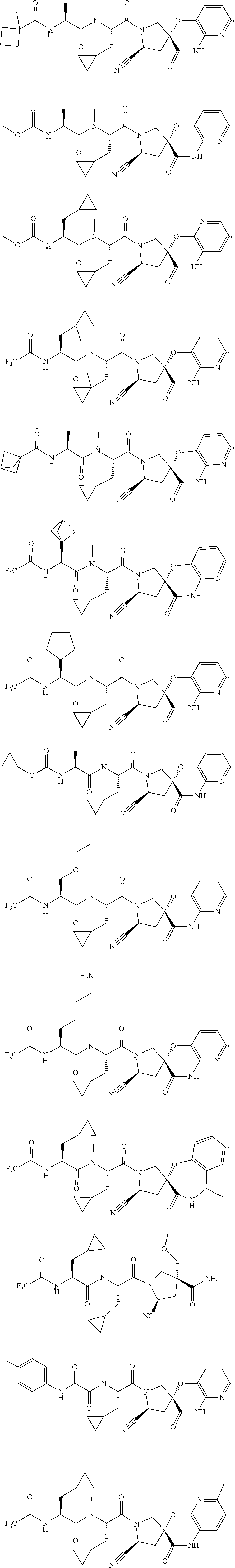 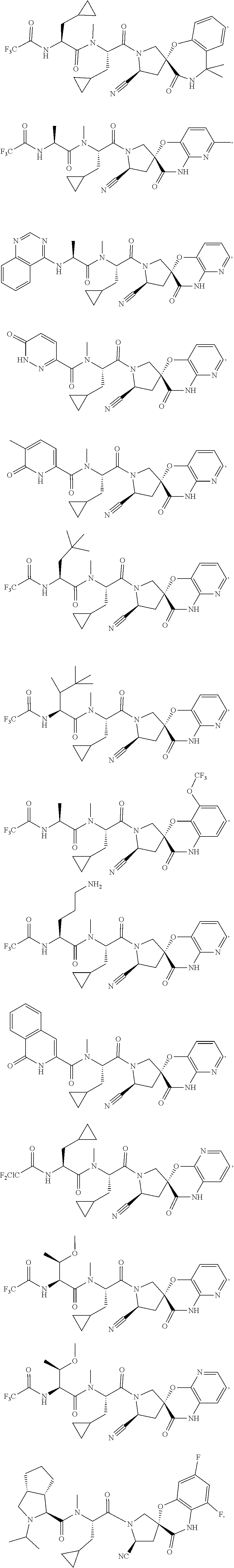 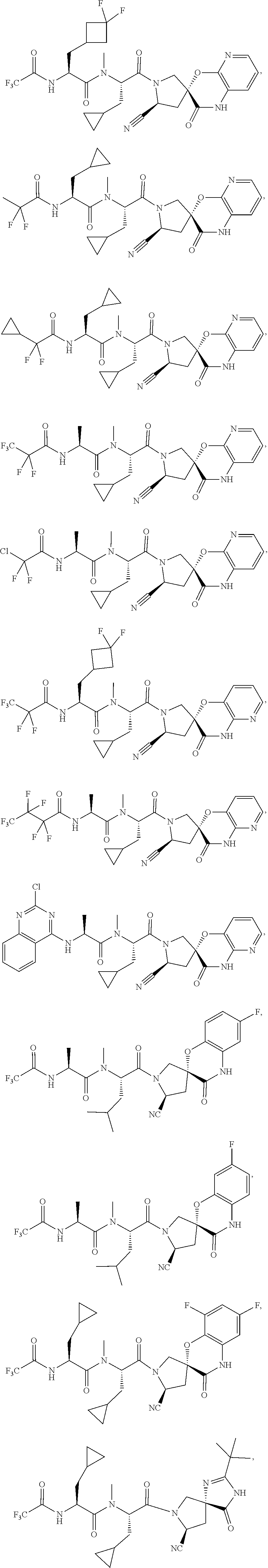 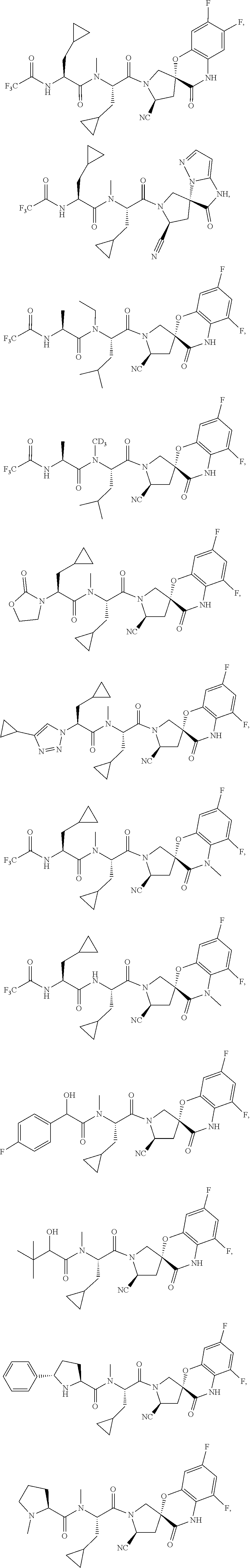 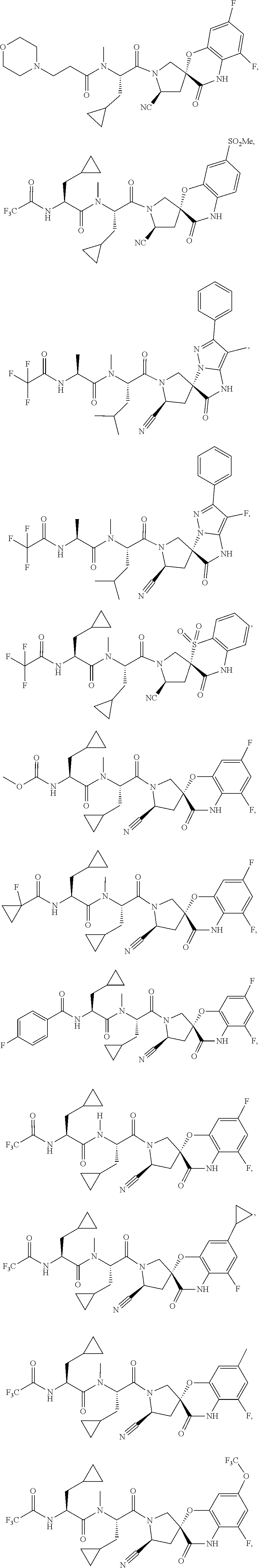 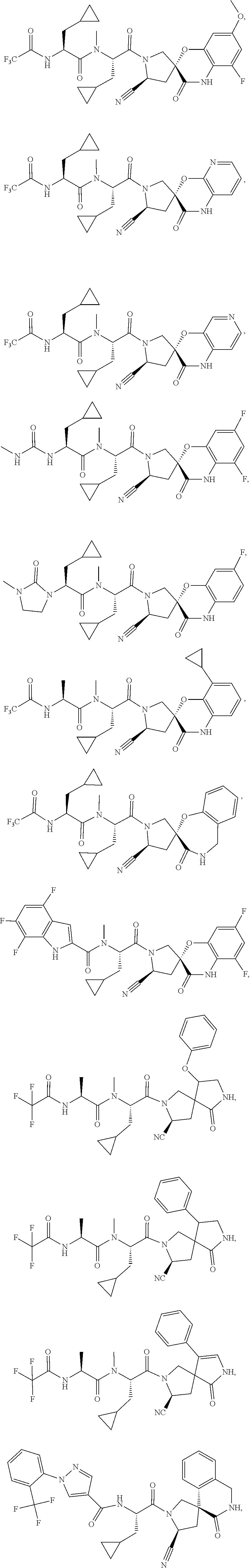 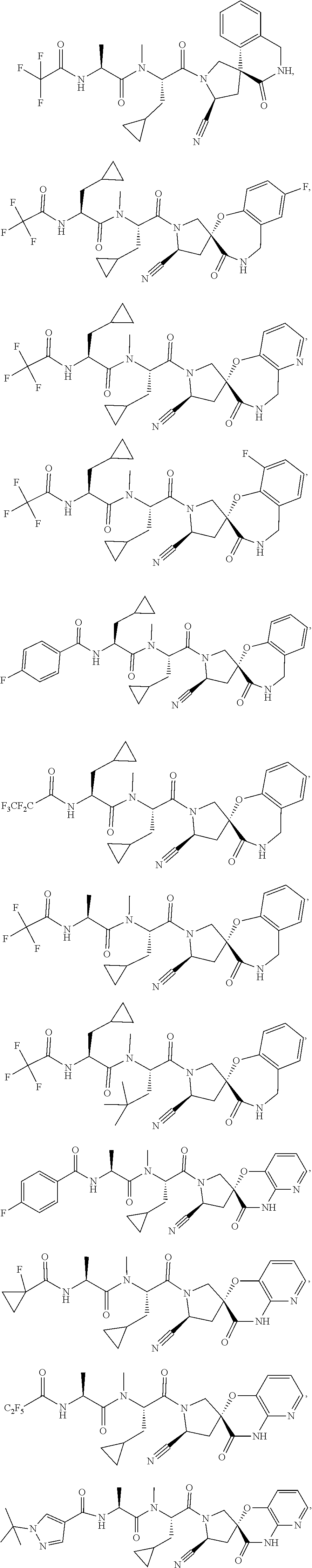 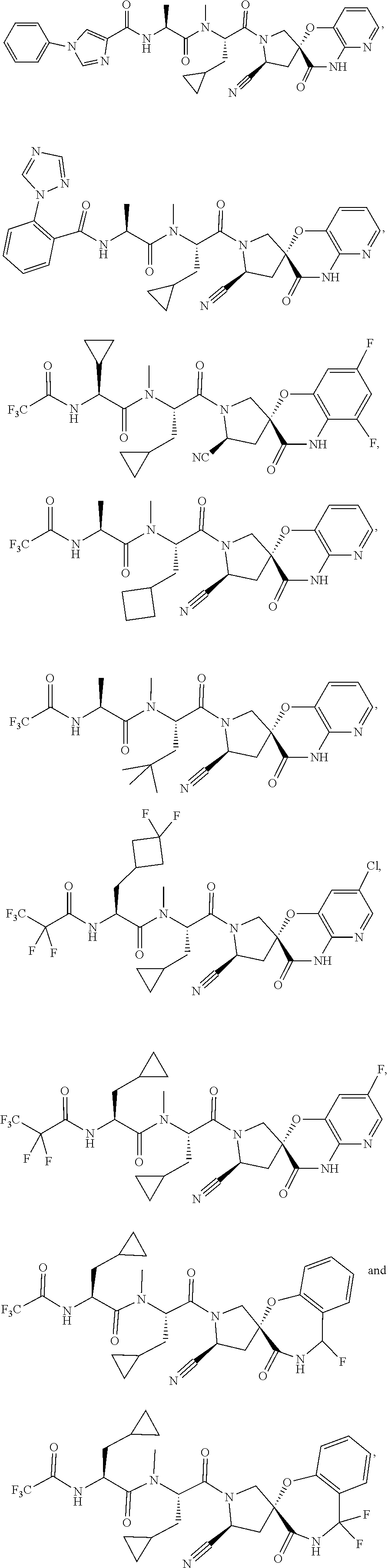
or a pharmaceutically acceptable salt of any of the foregoing.

125. A pharmaceutical composition comprising an effective amount of a compound of claim 1, or a pharmaceutically acceptable salt thereof, and excipient.

126.-141. (canceled)

142. A method for treating a coronavirus infection in a subject comprising administering to the subject in need thereof an effective amount of a compound of claim 1, or a pharmaceutically acceptable salt thereof.

143. The method of claim 142, further comprising administering an additional agent selected from the group consisting of an ACE inhibitor, an anticoagulant, an anti-inflammatory, an ARB, an ASO, a Covid-19 convalescent plasma, an entry inhibitor, an H₂ pump antagonist, an H-conducting channel, an HIV protease inhibitor, an HMG-CoA reductase inhibitor, an immune globulin, an immunosuppressant, an immunotherapeutic agent, a neuraminidase inhibitor, a nucleoside inhibitor, a nucleoside analog inhibitor, a polymerase inhibitor, a protease inhibitor, an siRNA, a statin, a tissue plasminogen activator, an antibiotic, an antimicrobial and a vaccine.

144.-158. (canceled)

159. A method for inhibiting a coronavirus protease comprising contacting a cell infected with a coronavirus with an effective amount of a compound of claim 1, or a pharmaceutically acceptable salt thereof, wherein the compound of claim 1, or a pharmaceutically acceptable salt thereof, selectively inhibits the coronavirus protease compared to a host protease.

160.-162. (canceled)
